(19) United States
(12) Patent Application Publication
(10) Pub. No.: US 2025/0213726 A1
(43) Pub. Date: Jul. 3, 2025

(54) THERAPEUTIC ADENO-ASSOCIATED VIRUS USING CODON OPTIMIZED NUCLEIC ACID ENCODING ALPHA-GLUCOSIDASE (GAA) FOR TREATING POMPE DISEASE, WITH SIGNAL PEPTIDE MODIFICATIONS

(71) Applicant: ASKLEPIOS BIOPHARMACEUTICAL, INC., Research Triangle Park, NC (US)

(72) Inventors: Anna TRETIAKOVA, Research Triangle Park, NC (US); Xavier AUGUELA MARTINEZ, Research Triangle Park, NC (US); Lester SUAREZ, Research Triangle Park, NC (US); Scott HAMMOND, Research Triangle Park, NC (US)

(73) Assignee: ASKLEPIOS BIOPHARMACEUTICAL, INC., Research Triangle Park, NC (US)

(21) Appl. No.: 18/848,389

(22) PCT Filed: Mar. 17, 2023
(86) PCT No.: PCT/US2023/015531

§ 371 Date: Sep. 18, 2024

Related U.S. Application Data
(60) Provisional application No. 63/444,804, filed on Feb. 10, 2023.
(60) Provisional application No. 63/348,862, filed on Jun. 3, 2022.
(60) Provisional application No. 63/321,336, filed on Mar. 18, 2022.

(51) Int. Cl. A61K 48/00; A61K 38/47; A61P 3/00; C12N 15/86

(52) U.S. Cl. CPC A61K 48/0058; A61K 38/47; A61P 3/00; C12N 15/86; C12Y 302/0102; C12N 2750/14143; C12N 2800/22; C12N 2830/008; C12N 2830/50

(57) ABSTRACT

Disclosed herein is a method for the treatment of Pompe Disease comprising administering a recombinant AAV (rAAV) vector comprising a rAVV genome comprising a heterologous nucleic acid encoding a GAA signal peptide or portion thereof, a heterologous signal peptide, and an acid alpha-glucosidase (GAA) polypeptide, or N-terminal truncation thereof, where the heterologous nucleic acid is operatively linked to a liver-specific promoter, where the nucleic acid encoding GAA polypeptide can be wild type nucleic acid sequence, or modified nucleic acid sequence, or a codon optimized nucleic acid sequence, and can optionally be modified to reduce or completely eliminate CG and CpG dinucleotides and, optionally eliminated alternative reading frames (ARF) content.

### CROSS-REFERENCE TO RELATED APPLICATIONS

This application claims benefit under 35 U.S.C. § 119(e) of U.S. Provisional Application No. 63/321,336 filed Mar. 18, 2022, U.S. Provisional Application No. 63/348,862 filed Jun. 3, 2022 and U.S. Provisional Application No. 63/444,804 filed Feb. 10, 2023, the contents of each of which are incorporated herein by reference in their entireties.

### SEQUENCE LISTING

The instant application contains a Sequence Listing as Table 9 herein, which is hereby incorporated by reference in its entirety.

### FIELD OF THE INVENTION

The present invention relates to methods to treat Pompe disease by administering adeno-associated virus (AAV) particles, virions and vectors for expression of an alpha-glucosidase (GAA) polypeptide, where the nucleic acid encoding GAA can be codon optimized or truncated. The compositions as disclosed herein can be used in methods to treat Pompe disease, including without the clinical need for administration of long-term GAA enzyme replacement therapy (ERT) for an extended period of time.

### BACKGROUND

Pompe disease (Glycogen storage disease type II; acid maltase deficiency; MIM 232300) is caused by recessive mutations of the GAA gene leading to complete or partial deficiency of the lysosomal enzyme acid α-glucosidase (GAA). Absence of GAA leads to the progressive accumulation of glycogen in the lysosomes of many tissues, particularly skeletal muscle and cardiomyocytes. Impaired energy metabolism then leads secondarily to severely disrupted muscle architecture, dysfunction, autophagy, and in adults, significant fatty replacement of skeletal muscle myocytes.

Clinically, the condition ranges from a fulminant infantile-onset Pompe disease (IOPD) typically leading to death before 12 months of age to a late-onset Pompe disease (LOPD), which is slowly progressive leading to myopathy causing loss of mobility and typically death from respiratory failure 5-15 years after diagnosis. Infantile-onset patients have cardiomyopathy often noted even at birth or even antenatally, with elevated creatine kinase (CK) and then within weeks to the first months of life develop severe hypotonia, respiratory insufficiency requiring ventilator support and massive cardiomegaly. Deaths are most often the result of cardiorespiratory failure, aspiration pneumonia or ventricular arrhythmias. Late-onset Pompe Disease (LOPD) patients (mostly adults, some juveniles) experience slowly progressive muscle weakness often leading to delayed diagnosis, extensive fatty replacement of trunk and proximal limb muscles, progressing to respiratory failure which is the primary cause of death (Carlier et al. 2011). Basilar artery aneurysms occur and can be life threatening if they rupture (El-Gharbawy et al. 2011; Hobson-Webb et al. 2012). As an alternative or adjunct to enzyme therapy, the feasibility of gene therapy approaches to treat GSD-II have been investigated (Amalfitano, A., et al., (1999) Proc. Natl. Acad. Sci. USA 96:8861-8866, Ding, E., et al. (2002) Mol. Ther. 5:436-446, Fraites, T. J., et al., (2002) Mol. Ther. 5:571-578, Tsujino, S., et al. (1998) Hum. Gene Ther. 9:1609-1616).

MYOZYME® (alglucosidase alfa) was the first US approved product (2006) for the treatment of Pompe disease; LUMIZYME® (alglucosidase alfa) was approved in 2010 and is the current standard-of-care (SOC) treatment for infantile-onset and late-onset Pompe patients. Alglucosidase alfa is administered intravenously every 2 weeks as an infusion at a dose of 20 mg/Kg (LUMIZYME Prescribing Information 2014). Alglucosidase alfa provides an exogenous source of GAA. Binding to mannose-6-phosphate receptors on the cell surface has been shown to occur via carbohydrate groups on the GAA molecule, after which it is internalized and transported into lysosomes, where it undergoes proteolytic cleavage that results in increased enzymatic activity. It then exerts enzymatic activity in cleaving glycogen. Although this enzyme replacement therapy (ERT) prolongs survival in most patients with infantile Pompe disease, a subset have either died, suffered ongoing muscle weakness or remained profoundly hypotonic requiring mechanical ventilator support despite compliance with SOC ERT. For late-onset patients, ERT moderately improves muscle function and pulmonary function parameters, initially, followed by stable function or decline and remains far from an ideal therapy (Schoser et al. 2017).

For infantile-onset patients, especially those with severe or null mutations (cross-reacting immune material [CRIM])-negatives), high and sustained anti-rhGAA Immunoglobulin G (IgG)-mediated immunity against the GAA enzyme is a primary reason for impaired or inadequate response to ERT. That is, ERT is known to provoke an antibody response in the form of both IgG and IgE and can also lead to infusion-associated reactions (Kishnani et al. 2007; Kishnani et al. 2010). Current practice is to initiate immune modulation with ERT for patients with LOPD at risk for antibody formation.

Additionally, enzyme replacement therapy (ERT) with alglucosidase alfa (MYOZYME®/LUMIZYME®) is delivered as an onerous every other week, or weekly infusion, and is the only treatment currently available. For those with infantile-onset Pompe disease (IOPD), GAA is absent (CRIM negative) or minimal (˜1% of normal) and causes rapidly progressive cardiorespiratory failure and death by the age of 2 years if left untreated (Parini et al. 2018). Subjects with a marked deficiency of GAA present as juveniles or adults (Late-Onset Pompe disease [LOPD]) with less severe symptoms and slower progression. Moreover, missing biweekly treatments can result in significant setbacks requiring many months of ERT to return to the same levels.

Accordingly, despite temporary therapeutic success, alglucosidase alfa ERT leaves a clear unmet medical need in both IOPD and LOPD. Longitudinal data in subjects confirm that ERT does not lead to complete correction or normalization of patients with Pompe disease. Ultimately subjects typically still decline, albeit at a slower rate, delaying the inevitable progression to death (Kuperus et al. 2017; Parini et al. 2018). While alglucosidase alfa prolongs survival for subjects with both IOPD and LOPD (LUMIZYME Prescribing Information, 2014) the antibody responses to the GAA and decline in effect poses several drawbacks.

Therefore, from a clinical perspective, long-term treatment of Pompe patients with ERT has had limited success. In the IOPD subset of patients, many experience high, sustained anti-rhGAA antibody titers (HSAT). Pompe patients who lack any residual GAA protein are deemed CRIM-negative. CRIM negative patients develop HSAT, and a subset of CRIM positive patients who also develop high or sustained intermediate titers suffer greatly increased mortality (Banugaria et al. 2011). Furthermore, the use of immunosuppression to prevent antibody formation in patients at risk for HSAT significantly prolonged survival and confirmed the clinical relevance of HSAT (Mendelsohn et al. 2009; Banugaria et al. 2011). Moreover, literature indicates that only about ˜1% of ERT is pharmacologically active.

Therefore, while enzyme therapy has demonstrated reasonable efficacy for severe infantile GSD II, the benefit of GAA enzyme therapy is limited by the need for frequent infusions as well as the subject developing inhibitor or neutralizing antibodies against recombinant hGAA protein (Amalfitano, A., et al. (2001) Genet. In Med. 3:132-138), there is a need for improved methods and alternative therapies to treat patients with Pompe disease. Adeno-associated virus (AAV) vector-mediated gene transfer provides an appropriate and feasible alternative.

### SUMMARY OF THE INVENTION

The technology described herein relates generally to a recombinant adenovirus associated (rAAV) vector comprising in its genome: (a) 5′ and 3′ AAV inverted terminal repeats (ITR) sequences, and (b) located between the 5′ and 3′ ITRs, a heterologous nucleic acid sequence encoding all or a portion of an endogenous GAA signal peptide, a heterologous signal peptide and an alpha-glucosidase (GAA) polypeptide, wherein the GAA polypeptide comprises amino acid residues 28-952 of SEQ ID NO: 1, 57-952 of SEQ ID NO: 1, or comprises a N-terminal GAA polypeptide fragment, such as comprising amino acids 28, 28-29, 28-30, 28-31, 28-32, or 28-33 of SEQ ID NO: 1 and a deletion of any number of amino acids from the next about 5 amino acids to about 40 amino acids after the N terminal GAA polypeptide fragment of SEQ ID NO: 1, and wherein the heterologous signal peptide can be inserted immediately at or after the N-terminal GAA polypeptide fragment and before the remaining amino acids of the GAA polypeptide, e.g., wherein the heterologous signal peptide is optionally fused at position 57 of the remaining amino acids of the GAA polypeptide, and where the GAA polypeptide can extend to amino acid 952 of SEQ ID NO: 1, or a functional fragment thereof, and wherein the nucleic acid sequence encoding the GAA polypeptide can be codon optimized, and wherein the heterologous nucleic acid is operatively linked to a liver-specific promoter. In certain embodiments, the homologous GAA signal peptide or a fragment thereof can be present.

A recombinant adenovirus associated (rAAV) vector comprising in its genome: (a) 5′ and 3′ AAV inverted terminal repeats (ITR) sequences, and (b) located between the 5′ and 3′ ITRs, optionally, a heterologous nucleic acid sequence encoding all or a portion of an endogenous GAA signal peptide, a heterologous signal peptide and an alpha-glucosidase (GAA) polypeptide, wherein the GAA polypeptide comprises amino acid residues 28-952 of SEQ ID NO: 1, amino acids 57-952 of SEQ ID NO: 1, or comprises a N-terminal GAA polypeptide fragment, for example, comprising amino acids 28, 28-29, 28-30, 28-31, 28-32, or 28-33 of SEQ ID NO: 1 and a deletion can be any number of amino acids from about 5 amino acids to about 40 amino acids after the N terminal GAA polypeptide fragment of SEQ ID NO: 1, and wherein the heterologous signal peptide can be inserted immediately before the remaining amino acids of the GAA polypeptide. In one embodiment, the heterologous signal peptide is optionally fused at position 57 of the remaining amino acids of the GAA polypeptide, and where the GAA polypeptide can extend to amino acid 952 of SEQ ID NO: 1, or a functional fragment thereof, and wherein the nucleic acid sequence encoding the GAA polypeptide can be wild-type or codon optimized, and wherein the heterologous nucleic acid is operatively linked to a liver-specific promoter.

In some embodiments, the nucleic acid sequence that encodes an endogenous GAA-signal peptide encodes at least 1-5, or at least 1-10, or at least 1-20, or at least about 1-23, or at least about 1-24, or at least about 1-25, or at least about 1-26, or at least about 1-27 concecutive amino acids of the endogenous GAA signal peptide of SEQ ID NO: 59. In some embodiments, the nucleic acid sequence encoding an GAA-signal peptide encodes a modified GAA signal peptide that comprises a deletion of at least 1, or at least 2, or at least 3, or at least 4, or at least 5, or at least 6, or at least 7, or at least 8, or at least 9, or at least 10, or at least 11, or at least 12, or at least 13, or at least 14, or at least 15, or at least 16, or at least 17, or at least 18, or at least 19, or at least 20 amino acids of SEQ ID NO: 59, where the deletions can be concecutive, or non-concecutive deletions.

In some embodiments, the nucleic acid sequence that encodes a GAA-signal peptide encodes at least 1-5, or at least 1-10, or at least 1-20, or at least about 1-23, or at least about 1-24, or at least about 1-25, or at least about 1-26, or at least about 1-27 concecutive amino acids of the endogenous GAA signal peptide.

In one embodiment of any aspect herein, there is codon optimized nucleic acid sequence encoding the GAA polypeptide is selected from the group consisting of SEQ ID NO: 1-18, or functional fragment thereofs.

In one embodiment of any aspect herein, the nucleic acid encoding SEQ ID NO: 3 is wildtype.

In one embodiment of any aspect herein, the vector comprises the nucleic acid sequence of SEQ ID NO: 23, or a functional variant thereof.

In one embodiment of any aspect herein, the heterologous nucleic acid sequence encodes a GAA protein comprising a signal peptide fused to the GAA polypeptide, wherein the signal peptide is an endogenous GAA signal peptide, or a heterologous signal peptide, or a combination thereof.

In one embodiment of any aspect herein, the AAV genome comprises, in the 5′ to 3′ direction: (a) a 5′ ITR, (b) a liver-specific promoter sequence, (c) an 5′ UTR sequence, (d) a nucleic acid encoding a portion or all of the endogenous GAA signal peptide, (e) a nucleic acid encoding a heterologous signal peptide or the N-terminal GAA polypeptide fragment, (f) a nucleic acid encoding an alpha-glucosidase (GAA) polypeptide, wherein the GAA polypeptide can be whole or a fragment thereof that is functionally active, (g) a poly A sequence, and (h) a reverse RNA pol II terminator sequence.

In one embodiment of any aspect herein, wherein the vector further comprises at least one of a UTR or a reverse RNA polII terminator sequence.

In one embodiment of any aspect herein, the UTR is 5′ or 3′.

In one embodiment of any aspect herein, the nucleic acid encoding the signal peptide encodes a signal sequence is selected from any of: an endogenous GAA signal peptide, a fibronectin signal peptide (FN1), a IL-2 wt signal peptide, modified IL-2 signal peptide, IL2(1-3) signal peptide, IgG signal peptide, a AAT signal peptide, a A2M signal peptide, or a PZP signal peptide, or an active fragment thereof having signal peptide activity.

In one embodiment of any aspect herein, the nucleic acid sequence encodes a GAA polypeptide having the amino acid sequence of SEQ ID NO: 1, or a polypeptide having at least 80% sequence identity to SEQ ID NO: 1 where amino acid residue 199 is a R (199R), amino acid residue 223 is a H (223H) and amino acid residue 780 is a I (780I).

In one embodiment of any aspect herein, the nucleic acid sequence encoding the GAA polypeptide is SEQ ID NO: 3, or a nucleic acid sequence having at least 80%, or at least 85%, or at least 90% sequence identity to SEQ ID NO: 3 that encodes a GAA polypeptide having at least 80% sequence identity to SEQ ID NO: 1 where amino acid residue 199 is a R (199R), amino acid residue 223 is a H (223H) and amino acid residue 780 is a I (780I).

In one embodiment of any aspect herein, the 5′ UTR sequence comprises SEQ ID NO: 41, or a nucleic acid having at least 80% sequence identity to SEQ ID NO: 41.

In one embodiment of any aspect herein, the 5′ UTR sequence comprises SEQ ID NO: 40, or a nucleic acid having at least 80% sequence identity to SEQ ID NO: 40.

In one embodiment of any aspect herein, the vector further comprises an intron sequence located 5′ of the nucleic acid sequence encoding the signal peptide, and 3′ of the promoter.

In one embodiment of any aspect herein, the intron sequence is selected from the group consisting of: MVM sequence, a HBB2 sequence, an CMVIE intron sequence, or a UBC intron sequence or a SV40 sequence.

In one embodiment of any aspect herein, the GAA polypeptide is a N-terminal truncated GAA polypeptide selected from any disclosed in Table 1.

In one embodiment of any aspect herein, the vector further comprises at least one polyA sequence located 3′ of the nucleic acid encoding the GAA gene and 5′ of the 3′ ITR sequence.

In one embodiment of any aspect herein, the heterologous nucleic acid sequence further comprises a 3′ UTR sequence, wherein the 3′ UTR sequence is located 3′ of the nucleic acid encoding the GAA polypeptide and 5′ of the 3′ ITR sequence, or is located between the nucleic acid encoding a GAA polypeptide and the poly A sequence, and can also comprise a RNA pol II terminator sequence.

In one embodiment of any aspect herein, the heterologous nucleic acid sequence further comprises a 3′ intron sequence, wherein the 3′ intron sequence is located 3′ of the nucleic acid encoding the GAA polypeptide and 5′ of the 3′ ITR sequence, or is located between the nucleic acid encoding the GAA polypeptide and a poly A sequence and/or a RNA polII terminator sequence.

In one embodiment of any aspect herein, the ITR comprises an insertion, deletion or substitution.

In one embodiment of any aspect herein, one or more CpG islands in the ITR are removed.

In one embodiment of any aspect herein, the nucleic acid encoding the signal peptide is selected from any of the group consisting of: AAT signal peptide (e.g., SEQ ID NO: 67), or an active fragment thereof having secretory signal activity, e.g., a nucleic acid encoding an amino acid sequence that has at least about 75%, or 80%, or 85%, or 90%, or 95%, or 98%, or 99% sequence identity to SEQ ID NO: 67; a fibronectin signal peptide (FN1) (e.g., SEQ ID NO: 73-75), or an active fragment thereof having secretory signal activity, e.g., a nucleic acid encoding an amino acid sequence that has at least about 75%, or 80%, or 85%, or 90%, or 95%, or 98%, or 99% sequence identity to SEQ ID NO: 73-75; an endogenous GAA signal peptide (SEQ ID NO: 51), or an active fragment thereof having secretory signal activity, e.g., a nucleic acid encoding an amino acid sequence that has at least about 75%, or 80%, or 85%, or 90%, or 95%, or 98%, or 99% sequence identity to SEQ ID NO: 51; an hIGF2 signal peptide (e.g., SEQ ID NO: 72), or an active fragment thereof having secretory signal activity, e.g., a nucleic acid encoding an amino acid sequence that has at least about 75%, or 80%, or 85%, or 90%, or 95%, or 98%, or 99% sequence identity to SEQ ID NO: 72; a IgG1 (201) signal peptide (SEQ ID NO: 54), or an active fragment thereof having secretory signal activity, e.g., a nucleic acid encoding an amino acid sequence that has at least about 75%, or 80%, or 85%, or 90%, or 95%, or 98%, or 99% sequence identity to SEQ ID NO: 54; wtIL2 leader peptide (SEQ ID NO: 55), or an active fragment thereof having secretory signal activity, e.g., a nucleic acid encoding an amino acid sequence that has at least about 75%, or 80%, or 85%, or 90%, or 95%, or 98%, or 99% sequence identity to SEQ ID NO: 55; mutant IL2 leader peptide (SEQ ID NO: 56) or an active fragment thereof having secretory signal activity, e.g., a nucleic acid encoding an amino acid sequence that has at least about 75%, or 80%, or 85%, or 90%, or 95%, or 98%, or 99% sequence identity to SEQ ID NO: 56; and the nucleic acid encoding the GAA polypeptide is selected from any of the group consisting of: SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18 or a nucleic acid sequence having at least 60%, or 70%, or 80%, 85% or 90% or 95%, or 98%, or 99% sequence identity to SEQ ID NOS: 1-18.

In one embodiment of any aspect herein, the nucleic acid encoding the GAA polypeptide is selected from SEQ ID NO: 3 or fragment thereof having functional GAA activity, or a nucleic acid sequence having at least 60%, or 70%, or 80%, 85% or 90% or 95%, or 98%, or 99% sequence identity to SEQ ID NO: 3 which encodes a GAA polypeptide at least 85% sequence identity to SEQ ID NO: 1 where amino acid residue 199 is a R (199R), amino acid residue 223 is a H (223H) and amino acid residue 780 is a I (780I).

In one embodiment of any aspect herein, the nucleic acid encoding the GAA polypeptide encodes a GAA polypeptide beginning at any of amino acid residues 35, 40, 50, 57, 60, 68, 69, 70, 72, 74, 779, 790, 791, 792, 793, or 796 of SEQ ID NO: 1 or a sequence 80% identical to SEQ ID NO: 1 where amino acid residue 199 is a R (199R), amino acid residue 223 is a H (223H) and amino acid residue 780 is a I (780I).

In one embodiment of any aspect herein, the GAA polypeptide has an endogenous GAA signal peptide or fragment thereof attached, and a heterologous signal peptide attached to or after the N-terminal of the GAA polypeptide, wherein the endogenous signal peptide has the amino acid sequence of SEQ ID NO: 59 or a sequence at least 80% sequence identity to SEQ ID NO: 59, and the heterologous signal peptide is selected from the group consisting of: SEQ ID NO: 60 (201 IgG signal peptide), or an IL2 wild type signal peptide (SEQ ID NO: 61), modified IL2 signal peptide (SEQ ID NO: 62), A2M signal peptide (SEQ ID NO: 63), or PZP signal peptide (SEQ ID NO: 64), or artificial signal peptide (SEQ ID NO: 65), or cathpetsin L signal peptide (SEQ ID NO: 66) or signal peptides at least 90% sequence identity to SEQ ID NOS: 60-66.

In one embodiment of any aspect herein, the liver specific promoter is selected from any of: SEQ ID NOS: 86, 88, 91-96, 146-150 or 439-441, or a liver specific promoter having at least 80% sequence identity to SEQ ID NOs: 86, 88, 91-96, 146-150 or 439-441.

In one embodiment of any aspect herein, the liver specific promoter is selected from any of: SEQ ID NOS: 98 or 99, or a liver specific promoter having at least 80% sequence identity to SEQ ID NOs: 98 or 99.

In one embodiment of any aspect herein, the liver specific promoter is SEQ ID NOS: 97, or a liver specific promoter having at least 80% sequence identity to SEQ ID NO: 97.

In one embodiment of any aspect herein, the recombinant vector is manufactured from the plasmid of SEQ ID NO: 27.

In one embodiment of any aspect herein, the nucleic acid comprises SEQ ID NO: 25, or a functional fragment thereof.

In one embodiment of any aspect herein, the recombinant AAV vector is a chimeric AAV vector, haploid AAV vector, a hybrid AAV vector or polyploid AAV vector.

In one embodiment of any aspect herein, the recombinant AAV vector is a rational haploid vector, a mosaic AAV vector, a chemically modified AAV vector, or a AAV vector from any AAV serotypes.

In one embodiment of any aspect herein, the recombinant AAV vector is selected from the group consisting of: a AAVXL32 vector, a AAVXL32.1 vector, a AAV8 vector, or a haploid AAV8 vector comprising at least one AAV8 capsid protein. In one embodiment of any aspect herein, the serotype is AAV3b. In one embodiment of any aspect herein, the AAV3b serotype comprises one or mutations in a capsid protein selected from any of: 265D, 549A, Q263Y. In one embodiment of any aspect herein, the AAV3b serotype is selected from any of: AAV3b265D, AAV3b265D549A, AAV3b549A or AAV3bQ263Y, or AAV3bSASTG.

In one embodiment of any aspect herein, the poly A sequence is a full length HGF poly A sequence. In one embodiment, it can be a functional fragment of the hGH polyA sequence.

In one embodiment of any aspect herein, the poly A sequence is selected from SEQ ID NO: 42, 43 or 44, or a nucleic acid sequence at least 80% sequence identity to SEQ ID NOS: 42-44.

In one embodiment of any aspect herein, the reverse RNA pol II terminator sequence comprises SEQ ID NO: 45, or a nucleic acid sequence at least 80% sequence identity to SEQ ID NO: 45. In one embodiment, it is those sequences.

Another aspect described herein provides a pharmaceutical composition comprising any of the recombinant AAV vectors described herein in a pharmaceutically acceptable carrier.

Another aspect described herein provides a method to treat a subject with Pompe Disease, or a glycogen storage disease type II (GSD II, Acid Maltase Deficiency) or having a deficiency in alpha-glucosidase (GAA) polypeptide, comprising administering any of the recombinant AAV vector, or any of the rAAV genome or nucleic acid sequence described herein to the subject.

In one embodiment of any aspect herein, the AAV vector manufactured from the plasmid of SEQ ID NO: 27.

In one embodiment of any aspect herein, the recombinant AAV vector comprises the nucleic acid sequence of SEQ ID NO: 3, or a functional fragment thereof.

In one embodiment of any aspect herein, the recombinant AAV vector comprises the nucleic acid sequence of SEQ ID NO: 23, or a functional variant thereof.

In one embodiment of any aspect herein, the GAA polypeptide is secreted from the subject's liver and there is uptake of the secreted GAA by skeletal muscle tissue, cardiac muscle tissue, diaphragm muscle tissue or a combination thereof, wherein uptake of the secreted GAA results in a reduction in lysosomal glycogen stores in the tissue(s).

In one embodiment of any aspect herein, the administering to the subject is selected from any of: intramuscular, sub-cutaneous, intraspinal, intracisternal, intrathecal, intravenous administration.

In one embodiment of any aspect herein, the recombinant AAV vector is a chimeric AAV vector, haploid AAV vector, a hybrid AAV vector or polyploid AAV vector.

In one embodiment of any aspect herein, the recombinant AAV vector is a rational haploid vector, a mosaic AAV vector, a chemically modified AAV vector, or a AAV vector from any AAV serotypes.

In one embodiment of any aspect herein, the recombinant AAV vector is a AAVXL32 vector or a AAVXL32.1 vector or a AAV8 vector, or a haploid AAV8 vector comprising at least one AAV8 capsid protein.

In one embodiment of any aspect herein, the recombinant AAV vector is a AAV8 vector.

In one embodiment of any aspect herein, the recombinant AAV vector is administered at a dosage range of between 1.0E9 vg/kg and 5.0E13 vg/kg. For example, 1.0E9 vg/kg and 5.0E12 vg/kg; 5.0E9 vg/kg and 5.0E12 vg/kg; 5.0E9 vg/kg and 1.0E12 vg/kg; 5.0E9 vg/kg and 5.0E11 vg/kg; 5.0E9 vg/kg and 5.0E10 vg/kg; and 1.0E9 vg/kg and 1.0E10 vg/kg.

In one embodiment of any aspect herein, the method further comprises receiving GAA protein enzyme replacement therapy, and withdrawing GAA protein enzyme replacement therapy (ERT) on the same day, a day after or, any time between day 1 and 26 weeks after administration of the recombinant AAV vector.

Another aspect described herein provides a nucleic acid construct comprising SEQ ID NO: 3, or a nucleic acid sequence at least 80% sequence identity to SEQ ID NOS: 3.

In one embodiment of any aspect herein, the expression of the nucleic acid sequence of SEQ ID NO: 3 or a nucleic acid having 80% sequence identity thereto encodes a GAA polypeptide having at least 80% sequence identity to SEQ ID NO: 1 and wherein there is R at position 199, a H at position 223 and I at position 780.

Another aspect described herein provides a nucleic acid construct comprising SEQ ID NO: 23, or a nucleic acid sequence at least 80% sequence identity to SEQ ID NO: 23.

In one embodiment of any aspect herein, the nucleic acid comprises SEQ ID NO: 3 or SEQ ID NO: 25, or a nucleic acid sequence at least 80% sequence identity to SEQ ID NOS: 3 or 25.

In one embodiment of any aspect herein, the expression of the nucleic acid sequence of SEQ ID NO: 3 or a nucleic acid having 80% sequence identity thereto encodes a GAA polypeptide having at least 80% sequence identity to SEQ ID NO: 1 and wherein there is R at position 199, a H at position 223 and I at position 780.

Another aspect described herein provides a recombinant AAV comprising any of the nucleic acid constructs described herein.

In one embodiment of any aspect herein, the AAV lacks at least 1 amino acids of the GAA N terminus.

In one embodiment of any aspect herein, the AAV lacks at least 5, 10, 15, 20, 25, 30, 35, 40, 45, 50, 55 or more amino acids of the GAA N terminus.

In one embodiment of any aspect herein, the heterologous signal peptide is inserted immediately after the endogenous GAA signal peptide or a potion thereof.

Aspects of the present invention teach certain benefits in construction and use which give rise to the exemplary advantages described below. Other features and advantages of aspects of the present invention will become apparent from the following more detailed description, taken in conjunction with the accompanying drawings, which illustrate, by way of example, the principles of aspects of the invention.

### BRIEF DESCRIPTION OF THE DRAWINGS

This application file contains at least one drawing executed in color. Copies of this patent application publication with color drawings will be provided by the Office upon request and payment of the necessary fee. The accompanying drawings illustrate aspects of the present invention. In such drawings:

FIGS. 1A-1D show serum GAA protein quantification by western blot densitometry. Serum GAA protein was measured by western blot densitometry at week 1 (FIG. 1A), week 2 (FIG. 1B). week 3 (FIG. 1C) and week 4 (cardiac serum) (FIG. 1D). Serum GAA was normalized to total protein (A-D).

FIGS. 2A-2D show serum GAA activity by 4MU assay. Serum GAA activity was measured by 4MU assay at week 1 (FIG. 2A), week 2 (FIG. 2B). week 3 (FIG. 2C) and week 4 (cardiac serum) (FIG. 2D).

FIGS. 3A-3C show target organ GAA protein quantification by western blot densitometry. GAA protein was measured 4 weeks post dosing by western blot densitometry in Heart (FIG. 3A), Diaphragm (FIG. 3B) and Liver (FIG. 3C). GAA was normalized to total protein.

FIGS. 4A-4D show target organ GAA activity by 4MU assay. GAA activity was measured by 4MU assay 4 weeks post dosing in Heart (FIG. 4A), Diaphragm (FIG. 4B), Quad (FIG. 4C) and Liver (FIG. 4D).

FIGS. 5A-5D show target organ Glycogen content. Glycogen content was measured 4 weeks post dosing in Heart (FIG. 5A), Diaphragm (FIG. 5B), Quad (FIG. 5C) and Liver (FIG. 5D).

FIGS. 6A-6C show serum GAA protein quantification by western blot densitometry for 3-week sacrifice animals. Serum GAA protein was measured by western blot densitometry at week 1 (FIG. 6A), week 2 (FIG. 6B) and week 3 (FIG. 6C). Serum GAA was normalized to total protein.

FIGS. 7A-7C show serum GAA activity by 4MU assay for 3-week sacrifice animals. Serum GAA activity was measured by 4MU assay at week 1 (FIG. 7A), week 2 (FIG. 7B) and week 3 (FIG. 7C).

FIGS. 8A-8C show target organ GAA activity by 4MU assay for 3-week sacrifice animals. GAA activity was measured by 4MU assay 3 weeks post dosing in Heart (FIG. 8A), Diaphragm (FIG. 8B) and Liver (FIG. 8C).

FIGS. 9A-9C show target organ Glycogen content for 3-week sacrifice animals. Glycogen content was measured 3 weeks post dosing in Heart (FIG. 9A), Diaphragm (FIG. 9B) and Liver (FIG. 9C).

FIGS. 10A-10H show serum GAA protein quantification by western blot densitometry for 8-week sacrifice animals. Serum GAA protein was measured by western blot densitometry at week 1 (FIG. 10A), week 2 (FIG. 10B). week 3 (FIG. 10C) and week 4 (FIG. 10D), week 5 (FIG. 10E), week 6 (FIG. 10F), week 7 (FIG. 10G), week 8 (FIG. 10H). Serum GAA was normalized to total protein.

FIGS. 11A-11H show serum GAA activity by 4MU assay for 8-week sacrifice animals. Serum GAA activity was measured by 4MU assay at week 1 (FIG. 11A), week 2 (FIG. 11B). week 3 (FIG. 11C) and week 4 (FIG. 11D), week 5 (FIG. 11E), week 6 (FIG. 11F), week 7 (FIG. 11G), week 8 (FIG. 11H).

FIGS. 12A-12C show target organ GAA protein quantification by western blot densitometry for 8-week sacrifice animals. GAA protein was measured 8 weeks post dosing by western blot densitometry in Heart (FIG. 12A), Diaphragm (FIG. 12B), and Liver (FIG. 12C).

FIGS. 13A-13C show target organ GAA activity by 4MU assay for 8-week sacrifice animals. GAA activity was measured by 4MU assay 8 weeks post dosing in Heart (FIG. 13A), Diaphragm (FIG. 13B) and Liver (FIG. 13C).

FIGS. 14A-14F show target organ Glycogen content for 8-week sacrifice animals. Glycogen content was measured 8 weeks post dosing in Heart (FIGS. 14A, 14D), Diaphragm (FIGS. 14B, 14E) and Liver (FIGS. 14C, 14F) and expressed normalized to dose per kg body weight (FIGS. 14D-14F).

FIG. 15 shows a schematic of the Actus, M3 and M4 constructs. The Actus comprises the liver promoter of SEQ ID NO: 97, the M3 construct is similar to the M2 construct, with the M3 construct comprising the promoter of SEQ ID NO: 99, whereas the M2 construction comprises the promoter of SEQ ID NO: 98, which comprises mutations within the muscle transcription factor binding site. The coding sequence in the M3 construct was modified to remove predicted alternative open reading frames and known immune stimulatory hexanucleotide CpG motifs. In the M4 construct, all CG dinucleotide were removed from the entire coding sequence. Alternative frames were not removed in selected M4 constructs. Amino acid sequence is identical to sequence found in Actus 101 (myozyme/lumizyme amino acid sequence) across the M3 and M4 constructs.

FIGS. 16A and 16B show analyses of hGAA target tissue uptake. FIG. 16A shows expression of GAA activity (top graph) following administrations of M4 as compared to Actus 101 and a vehicle control (VC) in the liver, heart and diaphragm, as well as glycogen levels (bottom graph). FIG. 16B shows expression of GAA activity (top graph) following administrations of M4 from two different lots as compared to Actus 101 and a vehicle control (VC) in the liver, heart and diaphragm, as well as glycogen levels (bottom graph).

FIGS. 17A and 17B show analyses of serum hGAA expressed by Actus 101 and M4. FIG. 17A shows protein expression of hGAA expressed from Actus 101, M4, and a vehicle control (VC). hGAA protein is 2.48 fold higher when expressed from M4 as compared to Actus 101. FIG. 17B shows total protein of hGAA activity present in the serum following administrations of M4 from two different lots as compared to Actus 101 and a vehicle control (VC) 4 weeks post injection.

FIGS. 18A-18C show performance of wild type (pM3-NCBI) & Actus 101 hGAA proteins in mouse heart at 4 weeks post vector injection. FIG. 18A is a bar graph showing the hGAA uptake following administration of the indicated constructs. FIG. 18B is a bar graph showing the hGAA activity following administration of the indicated constructs. FIG. 18C is a bar graph showing the glycogen levels following administration of the indicated constructs. Actus 101 performs better than the M3 construct in mouse hGAA activity (FIG. 18B) and glycogen reduction (FIG. 18C) in heart; this is in stark contrast to the M4 construct, which performs better than Actus 101.

FIG. 19 shows 4MU activity assay for hGAA activity at 4 weeks post transduction in mouse sera with the indicated construct. M4 performs better that Actus 101 in promoting GAA activity.

FIG. 20 shows liver retention observed following injection with saline (control), Actus 101, or M4. Liver retention appeared to be comparable between Actus 101 and M4.

FIGS. 21A-21D show analyses of hGAA target tissue uptake. FIG. 21A shows expression of GAA activity following administrations of M4, Actus 101 and a saline control in the heart. FIG. 21B shows expression of GAA activity following administrations of M4, Actus 101 and a saline control in the diaphragm. FIG. 21C shows expression of GAA activity following administrations of M4, Actus 101 and a saline control in the quadriceps muscle. FIG. 21D shows expression of GAA activity following administrations of M4, Actus 101 and a saline control in the soleus muscle.

FIG. 22 presents western blots showing the hGAA expression from vehicle control (VC), Actus 101, and various M4 constructs, Seg12 (SEQ ID NO: 30), Seq99 (SEQ ID NO: 29), Seq3 (SEQ ID NO:28), and Seq100 (SEQ ID NO: 27) at 7, 14 and 21 days post infection. Low and high doses (as indicated) were administered to GAA-KO mice. Levels were assessed at the days indicated. Seq100 results in a higher expression level that persists for a longer period of time as compared to VC, Actus 101 and the other indicated M4 constructs. “H ST 1” and “H ST 2” refer to HIGH DOSE STUDY 1 and HIGH DOSE STUDY 2, respectively. SEQ ID NO: 27, 28, 29, 30 are the plasmids for expression of the rAAV vectors expressing a GAA polypeptide having the sequence of SEQ ID NO: 1, where the rAAV vectors comprise codon optimized nucleic acid sequences selected from: SEQ ID NO: 3 (Seq100), SEQ ID NO: 4 (Seq3), SEQ ID NO:7 (Seq12), SEQ ID NO:13 (Seq99).

FIG. 23 shows hGAA levels in GAA-KO mouse at 21 days post administration of the indicated constructs. Seq100 results in a higher expression level of hGAA in the sera (top western blot) and liver (bottom western blot) at 21 days post administration as compared to VC, Actus 101 and the other indicated M4 constructs.

FIG. 24 shows normalized hGAA RNA levels in the liver of GAA-KO mice at 21 days post administration with the indicated constructs. Seq100 results in a higher expression level of hGAA RNA in the sera at 21 days post administration as compared to VC, Actus 101 and the other indicated M4 constructs.

FIG. 25 presents a western blot showing GAA uptake in the indicated target tissue (i.e., heart or diaphragm) at 21 days post administration with the indicated constructs. Seq100 results in a higher expression level of hGAA in each tissue at 21 days post administration as compared to VC, Actus 101 and the other indicated M4 constructs.

FIGS. 26A and 26B present bar graphs showing GAA and glycogen levels in the indicated tissue of GAA-KO mice at 21 days post administration FIG. 26A shows GAA activity in the indicated tissue of GAA-KO mice at 21 days post administration with the indicated constructs. Seq100 results in a higher expression level of hGAA in each tissue at 21 days post administration as compared to VC, Actus 101 and the other indicated M4 constructs. FIG. 26B shows glycogen in the indicated tissue of GAA-KO mice at 21 days post administration mice with the indicated constructs. Seq100 results in a higher level of glycogen clearance in each tissue at 21 days post administration as compared to VC, Actus 101 and the other indicated M4 constructs.

FIG. 27 presents a western blot showing hGAA expression in GAA-KO mice over time. Seq100 results in a higher expression level of hGAA in at day 7 and 14 post administration as compared to VC, Actus 101 and the other indicated M4 constructs.

FIG. 28 presents a bar graph showing the results of a 4MU assay in GAA-KO mice 21 days post administration. Seq100 results in a higher expression level of hGAA in at day 21 post administration as compared to VC, Actus 101 and the other indicated M4 constructs.

FIG. 29 is a schematic that shows a number of modified GAAs, showing a construct comprising (i) a GAA-signal peptide, or portion thereof, and (ii) a heterologous signal peptide, attached to (iii) a GAA polypeptide. A N-terminal truncated GAA polypeptide beginning at amino acid 57 is shown as an exemplary GAA polypeptide, however, any N-terminal truncated GAA polypeptide disclosed in Table 1 can be used.

FIG. 30 presents a western blot showing hGAA expression in GAA-KO mice 4 weeks following administration of the indicated constructs. Black arrow indicates GAA expression. Gray triangle indicates a non-specific band.

FIG. 31 presents a bar graph showing total GAA protein in serum of GAA-KO mice 4 weeks following administration of the indicated constructs. Saline is used a control.

FIG. 32 presents a bar graph showing total GAA activity in serum of GAA-KO mice 4 weeks following administration of the indicated constructs. Saline is used a control. 4MU activity in the serum is consistent with GAA expression levels.

FIG. 33 presents a bar graph showing total GAA activity in the heart of GAA-KO mice 4 weeks following administration of the indicated constructs. Saline is used a control. 4MU activity in the heart is consistent with GAA expression levels and expression of the construct achieved wild-type levels.

FIG. 34 presents a bar graph showing total glycogen levels in the heart of GAA-KO mice 4 weeks following administration of the indicated constructs. Saline is used a control. Glycogen levels in the heart is consistent with GAA expression levels and expression of the construct achieved wild-type levels.

FIG. 35A presents a western blot showing total GAA activity in the liver of GAA-KO mice 4 weeks following administration of the indicated constructs. FIG. 35B presents a bar graph showing total GAA activity in the heart of GAA-KO mice 4 weeks following administration of the indicated constructs. Saline is used a control. Reduced retention of modified GAA in the liver is observed.

FIG. 36 presents a table showing the level of GAA in serum, GAA activity in serum and heart, glycogen level in heart, and GAA levels retained in liver in mice following administration of indicated AAV (left column).

FIG. 37 presents a schematic of modified constructs. Mod-Actus is the Actus construct modified to remove wtAAV DNA sequences 5′ and 3′ to the ITRs. Mod-P072 is the P072 construct modified to replace the 5′UTR with a 5′UTR+intron sequence, remove the 3′UTR, and add a SV40 bi-directional polyA sequence. Mod-P092 is the P092 construct modified to replace the 5′UTR with a 5′UTR+intron sequence, remove the 3′UTR, and add a SV40 bi-directional polyA sequence. Mod-072 and mod-092 are also modified to remove wtAAV DNA sequences 5′ and 3′ to the ITRs.

FIGS. 38A and 38B present bar graphs showing GAA expression in huh7 cell culture for indicated AAVs. FIG. 38A shows GAA activity in huh7 cell lysates. FIG. 38B shows GAA activity in huh7 cell supernatants. Mod-P072 secretes that highlest level of GAA into the supernatant. Mod-Actus promotes that highest GAA activity in cells.

FIGS. 39A-39C present data showing in vivo expression of indicated AAVs at various levels in wild type mice (C57BL/6J). FIG. 39A is a graph showing GAA activity in serum at various weeks post administration in male mice. FIG. 39B is a graph showing GAA activity in serum at various weeks post administration in female mice. FIG. 39C is a graph showing GAA activity in serum at various weeks post administration in male and female mice (total). Mod-P072 achieves the highest GAA activity levels in serum of mice 4 weeks post administration.

FIGS. 40A-40C present bar graphs representing semi-quantitative analysis of GAA level western blots in liver, heart and quadriceps tissue following in vivo expression of indicated AAVs at various levels in wild type mice (C57BL/6J). FIG. 40A is a graph showing GAA levels in liver tissue in male and female mice. FIG. 40B is a graph showing GAA levels in heart tissue in male and female mice. FIG. 40C is a graph showing GAA levels in quadriceps tissue in male and female mice. Mod-Actus achieves the highest GAA uptake in liver tissue post administration. Mod-P072 achieves the highest GAA uptake in heart and quadriceps tissue post administration.

FIG. 41 present a bar graph glycogen levels in heart tissue following administration of Actus 101, pP110, and pP113 at the indicated dose. A greater reduction in glycogen in the cells was observed following administration of pP110 as compared to Actus 101 or pP113. Saline is used a control.

FIG. 42 present a bar graph glycogen levels in heart tissue following administration of M4, pP065, pP072 and pP092 at the indicated dose. Higher doses of pP065 and pP072 resulted in the greatest reduction of glycogen levels in the cell. Saline is used a control.

The above described figures illustrate aspects of the invention in at least one of its exemplary embodiments, which are further defined in detail in the following description. Features, elements, and aspects of the invention that are referenced by the same numerals in different figures represent the same, equivalent, or similar features, elements, or aspects, in accordance with one or more embodiments.

### DETAILED DESCRIPTION

The technology described herein is directed to recombinant AAV (rAAV) vectors and constructs for rAAV for delivering a GAA polypeptide to a subject in the methods to treat Pompe Disease, where the heterologous nucleic acid encoding GAA polypeptide is codon optimized to reduce an immune response and for enhanced and improved efficiency of expression in human subjects. That is, the rAAV constructs described herein for delivering a GAA polypeptide to a subject comprise improvements, such as but not limited to, a codon optimized nucleic acid sequence encoding a GAA polypeptide, where the codon optimized nucleic acid sequence encoding the GAA polypeptide is modified include features for example, (i) enhanced expression in vivo, (ii) to reduce CpG islands and/or to eliminate CG dinucleotide content, (iii) modification of STOP sequences or elimination of alternative reading frames (ARF), and (iv) to reduce the innate immune response. Or using a wildtype GAA modified to enhance expression. Furthermore, the rAAV constructs described herein for delivering a GAA polypeptide to a subject comprise improvements such as, e.g., incorporation of a 5′ UTR located between the nucleic acid expressing the GAA polypeptide and the liver specific promoter, and use of specific terminator sequences 3′ nucleic acid expressing the GAA polypeptide, such as, e.g., specific poly A sequences and/or terminator sequences, multiple polyA sequences, etc.

The technology described herein relates to improved recombinant AAV (rAAV) vectors and constructs for rAAV for delivering a GAA polypeptide to a subject in the methods to treat Pompe Disease to those previously disclosed in International Patent Application WO2020102645 and WO2021102107, both of which are incorporated herein in their entirety by reference.

In particular, described herein are targeted viral vectors, e.g., using rAAV vectors as an exemplary example, that comprise a nucleotide sequence containing inverted terminal repeats (ITRs), a liver specific promoter, a heterologous gene, a poly-A tail and potentially other regulator elements for use to treat Pompe disease, where the heterologous gene is human GAA, and wherein the vector, e.g., rAAV can be administered to a patient in a therapeutically effective dose that is delivered to the appropriate tissue and/or organ for expression of the heterologous gene and treatment of the disease, e.g., Pompe disease.

One aspect described herein provides a recombinant adenovirus associated (AAV) vector comprising in its genome: (a) 5′ and 3′ AAV inverted terminal repeats (ITR) sequences, and (b) located between the 5′ and 3′ ITRs, a heterologous nucleic acid sequence encoding a polypeptide comprising an alpha-glucosidase (GAA) polypeptide, wherein the heterologous nucleic acid is operatively linked to a liver-specific promoter as disclosed herein.

In one embodiment of any aspect herein, the heterologous nucleic acid sequence encodes a GAA polypeptide comprising a secretory signal fused to the GAA polypeptide, wherein the secretory signal (also referred to herein as “signal peptide”) is the endogenous GAA polypeptide, or an exogenous GAA polypeptide.

In one embodiment of any aspect herein, the nucleic acid sequence encoding the GAA polypeptide is the human GAA gene or a human codon optimized GAA gene (coGAA) or a modified GAA nucleic acid sequence. As disclosed herein, the nucleic acid encoding the human GAA protein is selected from any of SEQ ID NO: 1-18, or a functional variant having least 80%, or at least about 85%, or at least about 90%, or at least about 95%, or at least about 98% sequence identity to any of SEQ ID NO: 1-18.

In some embodiments, GAA expressed that comprises at least a signal peptide that promotes secretion of GAA polypeptide from the liver. In some embodiments, the GAA polypeptide, or modified GAA, is expressed as a fusion protein comprising at least a signal peptide that promotes secretion of the GAA polypeptide from the liver.

In all aspects of all embodiments of the technology described herein, the liver specific promoter expresses the hGAA polypeptide preferentially in the liver. In all aspects of all embodiments of the technology described herein, in some embodiments where the AAV vector comprises at least one capsid protein targeting the liver.

### I. Recombinant AAV Expressing GAA

As disclosed herein, one aspect of the technology relates to a method to treat Pompe disease using a rAAV vector comprising a capsid, and within its capsid, a nucleotide sequence referred to as the “rAAV vector genome”. The rAAV vector genome (also referred to as “rAAV genome) includes multiple elements, including, but not limited to two inverted terminal repeats (ITRs, e.g., the 5′-ITR and the 3′-ITR), and located between the ITRs are additional elements, including a promoter, a heterologous gene encoding a GAA polypeptide and a poly-A tail, where the heterologous gene encoding a GAA polypeptide is codon optimized, e.g., including but not limited to, reducing CpGs, reduced CpG islands, and minimizing or eliminating internal start codons.

In some embodiments, the rAAV genome disclosed herein comprises a 5′ ITR and 3′ ITR sequence, and located between the 5′ITR and the 3′ ITR, a promoter, e.g., a liver specific promoter sequence as disclosed herein, which operatively linked to a heterologous nucleic acid encoding a nucleic acid encoding an alpha-glucosidase (GAA) polypeptide, where the heterologous nucleic acid is codon optimized as disclosed herein, and where there is a 5′ UTR located between the nucleic acid encoding a GAA polypeptide and the liver specific promoter sequence. In some instances, there is also a 3′ UTR. Further, the UTR can contain an intron. In one embodiment, the heterologous nucleic acid sequence can optionally further comprise one or more of the following elements: an intron sequence, a nucleic acid encoding a secretory signal peptide, which can be an endogenous signal peptide (SP) or a heterologous SP as disclosed herein, a poly A sequence, and a terminator sequence. In some embodiments, the 5′ UTR sequence comprises SEQ ID NO: 41, or comprises SEQ ID NO: 40, or a sequence having at least 85%, or at least 90% or more sequence identity to SEQ ID NOs: 40 or 41. In some embodiments, the poly A sequence is a full length HGH poly A sequence comprising SEQ ID NO: 42, or a sequence having at least 85%, or at least 90% or more sequence identity to SEQ ID NO: 42. In some embodiments, the terminator sequence is a reverse RNA pol II terminator sequence. In some embodiments, a reverse RNA pol II terminator sequence comprises sequence SEQ ID NO: 45, or a sequence having at least 85%, or at least 90% or more sequence identity to SEQ ID NO: 45.

In some embodiments, the nucleic acid encoding an alpha-glucosidase (GAA) polypeptide encodes a full-length GAA polypeptide, e.g., beginning at residue 28 of SEQ ID NO: 1. In some embodiments, the nucleic acid encoding an alpha-glucosidase (GAA) polypeptide encodes a truncated GAA polypeptide, such as, for example, beginning at amino acid residues 35, 40, 50, 57, 60, 68, 69, 70, 72, 74 and/or a C-terminal truncation beginning at residues 779, 790, 791, 792, 793 and 796 of SEQ ID NO: 1, or a GAA polypeptide that has at least 80%, or at least 85%, or at least 90% or at least 95% sequence identity to SEQ ID NO: 1 over the amino acid residues 35-952, 40-952, 50-952, 57-952, 60-952, 68-952, 69-952, 70-952, 72-952, 74-952, 779-952, 790-952, 791-952, 792-952, 793-952 and 796-952 of SEQ ID NO: 1.

In some embodiments, the nucleic acid encoding an alpha-glucosidase (GAA) polypeptide encodes a full-length GAA polypeptide (e.g., residues 28-952 of SEQ ID NO: 1), or a truncated GAA polypeptide, e.g., a GAA polypeptide beginning at any of residues 35, 40, 50, 57, 60, 68, 69, 70, 72, 74 of SEQ ID NO: 1 and/or a second truncation starting at residues 779, 790, 791, 792, 793 and 796 of SEQ ID NO: 1) that has an endogenous GAA signal peptide attached to the N-terminal of the GAA polypeptide, e.g., comprises endogenous signal peptide comprising residues of SEQ ID NO: 59. For example, SEQ ID NO: 1 and only a heterologous or homologous signal peptide. In alternative embodiments, the nucleic acid encoding an alpha-glucosidase (GAA) polypeptide encodes a full-length GAA polypeptide (e.g., residues 28-952 of SEQ ID NO: 1), or a truncated GAA polypeptide, e.g., a GAA polypeptide beginning at any of residues 35, 40, 50, 57, 60, 68, 69, 70, 72, 74, 779, 790, 791, 792, 793 and 796 of SEQ ID NO: 1, that has a heterologous signal peptide attached to the N-terminal of the full-length, or truncated GAA polypeptide.

In alternative embodiments, the nucleic acid encoding an alpha-glucosidase (GAA) polypeptide encodes a full-length GAA polypeptide (e.g., residues 28-952 of SEQ ID NO: 1), or a N-terminal truncated GAA polypeptide, e.g., a GAA polypeptide beginning at any of residues 35, 40, 50, 57, 60, 68, 69, 70, 72, 74, 779, 790, 791, 792, 793 and 796 of SEQ ID NO: 1, and also encodes a GAA-signal peptide or a portion or fragment thereof, and a heterologous signal peptide attached to the N-terminal of the full-length, or truncated GAA polypeptide. In some embodiments, the GAA polypeptide is a N-terminal truncated GAA polypeptide, with the truncation beginning at any amino acids 29-35 of SEQ ID NO: 1. In some embodiments, the nucleic acid encoding an alpha-glucosidase (GAA) polypeptide comprises (i) a GAA-signal peptide, or a portion thereof, e.g., a N-terminal portion thereof, (ii) a portion of the GAA polypeptide, (e.g., any portion of residues 28-56 of SEQ ID NO: 1) (iii) a heterologous signal peptide as disclosed herein, and (iv) a GAA polypeptide, e.g., a N-terminal truncated GAA polypeptide, e.g., a GAA polypeptide beginning at any of residues 35, 40, 50, 57, 60, 68, 69, 70, 72, 74, 779, 790, 791, 792, 793 and 796 of SEQ ID NO: 1. In some embodiments, the GAA polypeptide can comprise a C-terminal truncation of at least 1, or at least 2, or at least 3, or at least 4, or at least 5, or at least between 5-10, or between 10-20, or between 20-30 amino residues at the C-terminus of the GAA polypeptide. Exemplary heterologous signal peptides are disclosed herein, including, but not limited to, signal peptides comprising amino acids selected from any of SEQ ID NO: 60-78. In some embodiments, the signal peptide attached to the N-terminal of SEQ ID NOs: 60, 61, 62, 63 and 64.

### A. Alpha-Glucosidase (GAA) Polypeptide

The GAA gene (NM_000152.3) is approximately 18.3 kilobases (kb) long and contains 20 exons (Dasouki et al. 2014). Its complementary DNA has 2,859 nucleotides of coding sequence which encode the immature 952 amino acid enzyme. GAA is synthesized as a membrane bound, catalytically inactive (with respect to the natural substrate glycogen) precursor which is sequestered in the endoplasmic reticulum. It undergoes sugar chain modification in the Golgi complex, followed by transport into the (minor) secretory pathway, or into lysosomes where it is trimmed in a stepwise process at both the amino- and carboxyl-termini. Phosphorylation of mannose residues ensures efficient transport of the enzyme to the lysosomes via the mannose 6-phosphate receptor. In the lysosomes, GAA catalyzes the hydrolysis of α1→4 glucosidic linkages in glycogen in the low potential hydrogen (pH) environment to glucose. Specificity for the natural substrate (glycogen) is gained during its maturation.

Many normal allelic variants exist in GAA and are responsible for the three known alloenzymes (GAA1, GAA2, and GAA4). More than 450 mutations in GAA have been reported in individuals with Pompe disease. Nonsense mutations, large and small gene rearrangements, and splicing defects have been observed with many mutations being potentially specific to families, geographic regions, or ethnicities. Combinations of mutations that result in either complete or nearly complete absence of GAA enzyme activity (typically <1% of normal activity in skin fibroblasts) are seen more commonly in individuals with IOPD, whereas those combinations that allow partial enzyme activity (approximately 2-40% of normal activity in skin fibroblasts) typically have LOPD presentation. GAA mutations result in messenger RNA instability and/or severely truncated acid α-glucosidase or an enzyme with markedly decreased activity. Dysfunction or absence of GAA leads to the accumulation of glycogen in lysosomes and in the cytoplasm in multiple tissues, resulting in the destruction of skeletal, smooth and cardiac muscle. The effect of the enzyme deficiency may extend to vesicle systems that are linked to lysosomes and may also affect receptors, such as glucose transporter 4, that cycle through these organelles. Evidence has also shown a failure of productive autophagy and the progressive accumulation of autophagosomes that disrupt the contractile apparatus in muscle fibers, which correlated with a lack of correction of skeletal muscle during ERT.

Alpha-glucosidase (GAA) polypeptide is a member of family 31 of glycoside hydrolyases. Human GAA is synthesized as a 110 kDal precursor (Wisselaar et al. (1993) J. Biol. Chem. 268(3): 2223-31). The mature form of the enzyme is a mixture of monomers of 70 and 76 kDal (Wisselaar et al. (1993) J. Biol. Chem. 268(3): 2223-31). The precursor enzyme has seven potential glycosylation sites and four of these are retained in the mature enzyme (Wisselaar et al. (1993) J. Biol. Chem. 268(3): 2223-31). The proteolytic cleavage events which produce the mature enzyme occur in late endosomes or in the lysosome (Wisselaar et al. (1993) J. Biol. Chem. 268(3): 2223-31).

The rAAV vector genome can encode a GAA polypeptide can include, for example, amino acid residues 40-952 of human GAA, or a smaller portion, such as amino acid residues 40-790.

The C-terminal 160 amino acids are absent from the mature 70 and 76 kDal GAA polypeptide species. However, certain Pompe alleles resulting in the complete loss of GAA activity map to this region, for example Val949Asp (Becker et al. (1998) J. Hum. Genet. 62:991). The phenotype of this mutant indicates that the C-terminal portion of the protein, although not part of the 70 or 76 kDal species, plays an important role in the function of the protein. It has also been reported that the C-terminal portion of the protein, although cleaved from the rest of the protein during processing, remains associated with the major species (Moreland et al. (Nov. 1, 2004) J. Biol. Chem., Manuscript 404008200). Accordingly, the C-terminal residues could play a direct role in the catalytic activity of the protein, and/or may be involved in promoting proper folding of the N-terminal portions of the protein.

The native GAA gene encodes a precursor polypeptide which possesses a signal sequence and an adjacent putative trans-membrane domain, a trefoil domain (PFAM PF00088) which is a cysteine-rich domain of about 45 amino acids containing 3 disulfide linkages (Thim (1989) FEBS Lett. 250:85), the domain defined by the mature 70/76 kDal polypeptide, and the C-terminal domain. It has been reported that both the trefoil domain and the C-terminal domain are required for the production of functional GAA, and that it is possible that the C-terminal domain interacts with the trefoil domain during protein folding perhaps facilitating appropriate disulfide bond formation in the trefoil domain.

In some embodiments of the methods and compositions as disclosed herein, the human GAA protein expressed by the AAV comprises amino acids of SEQ ID NO: 1, or a protein at least 60%, or 70%, or 80%, 85% or 90% or 95%, or 98%, or 99% identical to SEQ ID NO: 1.

In some embodiments, the hGAA polypeptide comprises a signal peptide (SP). One of ordinary skill in the art can appreciate particular positions of GAA to which a signal peptide (SP) can be fused.

In some embodiments of the methods and compositions as disclosed herein, the human GAA protein expressed by the AAV comprises amino acids of SEQ ID NO: 1, or fragments or variants thereof, for example a human GAA protein beginning at any of residues selected from: 40, 50, 57, 68, 69, 70, 71, 72, 779, 787, 789, 790, 791, 792, 793, or 796 of SEQ ID NO: 1. Accordingly, in one aspect the invention relates to a GAA protein, where the SP is fused to N-terminal amino acid 40, 50, 57, 68, 69, 70, 71, 72, 779, 787, 789, 790, 791, 792, 793, or 796 of human GAA of SEQ ID NO: 1. In some embodiments, the GAA polypeptide expressed by the AAV vector disclosed herein has at least 80%, or at least 85%, or at least 90% or at least 95% sequence identity to amino acid residues selected from: 28-952, 35-952, 40-952, 50-952, 57-952, 60-952, 68-952, 69-952, 70-952, 72-952, 74-952, 779-952, 790-952, 791-952, 792-952, 793-952 and 796-952 of SEQ ID NO: 1, and can further comprise a N-terminal signal peptide, where the signal peptide can be an endogenous GAA signal peptide, or a heterologous signal peptide as disclosed herein.

In some embodiments of the methods and compositions as disclosed herein, the human GAA protein expressed by the AAV comprises amino acids is a human GAA protein beginning at any of residues selected from: 40, 50, 57, 68, 69, 70, 71, 72, 779, 787, 789, 790, 791, 792, 793, or 796 of SEQ ID NO: 1, or a protein at least 60%, or 70%, or 80%, 85% or 90% or 95%, or 98%, or 99% identical thereto.

(i) Modified GAA (modGAA) Polypeptides

In some embodiments, the modified human GAA protein comprises a polypeptide with at least one modification selected from: H199R, R223H, V780I or H201L of SEQ ID NO: 1, or a variant of at least 80%, 90%, 95%, or 99% homology to at least 500, 550, 600, 650, 700, 750, 800, 850, or 900 amino acids of SEQ ID NO: 1 having at least one of these modification. In some embodiments, the modified human GAA protein comprises a polypeptide comprises at least two modifications selected from: H199R, R223H, V780I or H201L of SEQ ID NO: 1, or a variant of at least 80%, 90%, 95%, or 99% homology to at least 500, 550, 600, 650, 700, 750, 800, 850, or 900 consecutive amino acids of SEQ ID NO: 1 having at least two of these modifications. In some embodiments, the modified human GAA protein comprises a polypeptide with three modifications selected from: H199R, R223H, V780I and H201L of SEQ ID NO: 1 (GAA-H199R-H201L-R223H or GAA-H199R-H201L-V780I), or a variant of at least 80%, 90%, 95%, or 99% homology to at least 500, 550, 600, 650, 700, 750, 800, 850, or 900 concecutive amino acids of SEQ ID NO: 1 having these three modifications.

One can use these modified GAA polypeptides and fragments thereof. In some embodiments, the human modified GAA protein expressed by the AAV comprises a GAA polypeptide of SEQ ID NO: 1 as modified above, or a protein at least 60%, or 70%, or 80%, 85% or 90% or 95%, or 98%, or 99% identical to SEQ ID NO: 1, or a nucleic acid encoding such a sequence (SEQ ID NO: 3), or a fragment of SEQ ID NO: 1, wherein the fragment begins at any of residues selected from: 40, 50, 57, 68, 69, 70, 71, 72, 779, 787, 789, 790, 791, 792, 793, or 796 of any of SEQ ID NO: 1 (modGAA; H199R, R223H, V780I) or a protein at least 60%, or 70%, or 80%, 85% or 90% or 95%, or 98%, or 99% identical to SEQ ID NO: 1, where there is R at amino acid position 199; a H at amino acid position 223; and I at amino acid position 780. Actus 101 (SEQ ID NO: 1), while comprising three point mutations, is considered a wildtype GAA.

In some embodiments, GAA is modified to add or remove glycosylation sites such as N-linked glycosylation sites, O-linked glycosylation sites or both. In certain embodiments, the addition or removal of glycosylation sites are achieved by N-terminal deletions, C-terminal deletions, internal deletions, random point mutagenesis, or, site directed mutagenesis. In some embodiments, the exemplary GAA modification involve addition of one or more Asparagine (Asn) residue/s or, one or more mutation to yield Asparagine (Asn) residue/s or, deletion of one or more Asparagine (Asn) residue/s. In certain embodiments, all or some of the N-linked, and/or, O-linked glycosylation sites present in GAA are mutated. In some embodiments, GAA modifications will yield information pertaining to the biological activity, physical structure and/or substrate binding potential of GAA.

In one embodiment, the modified human GAA protein comprises a deletion of the stretch of amino acids between and inclusive of 29-56 of SEQ ID NO: 1. In one embodiment, a modified human GAA protein comprising a deletion of the stretch of amino acids between and inclusive of 29-56 of SEQ ID NO: 1 is no longer maintained within the cell.

In one embodiment, the modified human GAA protein comprises a polypeptide with at least one modification selected from Table 12. Modifications listed on Table 12 are commonly identified GAA polymorphisms but are not associated with a disease, e.g., Pompe disease.

TABLE 12
Common GAA polymorphisms.

| Position(S) | Amino Acid No | Mutation |
|---|---|---|
| CA401359830 | 2 | G > E |
| CA294886445 | 3 | V > A |
| CA401359856 | 4 | R > S |
| CA401359866 | 5 | H > P |
| CA401359886 | 6 | P > Q |
| CA401359883 | 6 | P > S |
| CA401359899 | 7 | P > T |
| CA8814758 | 8 | C > Y |
| CA401359938 | 9 | S > C |
| CA10604542 | 9 | S > P |
| CA401359943 | 10 | H > Y |
| CA8814761 | 11 | R > L |
| CA8814763 | 12 | L > F |
| CA8814764 | 12 | L > P |
| CA401360004 | 14 | A > V |
| CA8814770 | 16 | C > S |
| CA401360048 | 17 | A > S |
| CA8814773 | 17 | A > V |
| CA401360057 | 18 | L > F |
| CA401360075 | 19 | V > L |
| CA8814774 | 19 | V > M |
| CA8814775 | 20 | S > F |
| CA401360089 | 20 | S > T |
| CA401360111 | 22 | A > T |
| CA8814776 | 22 | A > V |
| CA8814777 | 23 | T > A |
| CA294886529 | 24 | A > V |
| CA8814782 | 25 | A > V |
| CA8814784 | 27 | L > V |
| CA401360200 | 28 | G > E |
| CA294886548 | 28 | G > R |
| CA10604460 | 28 | G > V |
| CA294886572 | 29 | H > Y |
| CA401360231 | 31 | L > V |
| CA8814786 | 33 | H > R |
| CA8814788 | 34 | D > Y |
| CA401360282 | 39 | P > L |
| CA8814790 | 39 | P > S |
| CA401360310 | 44 | G > D |
| CA401360312 | 45 | S > A |
| CA401360316 | 45 | S > C |
| CA401360317 | 45 | S > F |
| CA401360314 | 45 | S > P |
| CA401360321 | 46 | S > F |
| CA294886602 | 47 | P > S |
| CA294886615 | 51 | E > G |
| CA8814796 | 53 | H > N |
| CA8814797 | 53 | H > R |
| CA8814799 | 54 | P > L |
| CA8814798 | 54 | P > T |
| CA294886644 | 56 | H > L |
| CA294886642 | 56 | H > N |
| CA8814800 | 56 | H > Y |
| CA294886669 | 58 | Q > H |
| CA8814803 | 59 | G > E |
| CA8814802 | 59 | G > R |
| CA401360401 | 60 | A > D |
| CA401360398 | 60 | A > T |
| CA401360410 | 61 | S > R |
| CA401360414 | 62 | R > K |
| CA401360423 | 63 | P > L |
| CA294886682 | 64 | G > E |
| CA401360435 | 65 | P > L |
| CA401360432 | 65 | P > S |
| CA8814806 | 69 | Q > * |
| CA8814807 | 69 | Q > R |
| CA8814808 | 70 | A > E |
| CA401360459 | 70 | A > P |
| CA8814810 | 71 | H > Q |
| CA294886764 | 72 | P > T |
| CA8814813 | 73 | G > S |
| CA401360481 | 74 | R > C |
| CA8814815 | 74 | R > P |
| CA8814817 | 77 | A > P |
| CA401360499 | 77 | A > V |
| CA8814819 | 79 | P > S |
| CA401360524 | 81 | Q > H |
| CA401360526 | 82 | C > R |
| CA8814823 | 83 | D > V |
| CA401360542 | 84 | V > G |
| CA401360539 | 84 | V > L |
| CA294886928 | 86 | P > H |
| CA8814830 | 87 | N > H |
| CA8814831 | 87 | N > T |
| CA401360560 | 88 | S > G |
| CA401360566 | 88 | S > R |
| CA8814834 | 90 | F > L |
| VAR_004285 | 91 | D > N |
| CA241285 | 91 | D > S |
| CA8814836 | 91 | D > Y |
| CA401360596 | 93 | A > V |
| CA401360598 | 94 | P > S |
| CA401360604 | 95 | D > N |
| CA401360622 | 97 | A > V |
| CA401360637 | 99 | T > I |
| CA8814843 | 100 | Q > E |
| CA401360641 | 100 | Q > K |
| CA401360648 | 100 | Q > R |
| CA401360674 | 102 | Q > * |
| CA401360696 | 103 | C > W |
| CA8814847 | 105 | A > D |
| CA8814848 | 105 | A > V |
| CA401360722 | 106 | R > S |
| CA294887102 | 107 | G > C |
| CA401360737 | 107 | G > D |
| CA401360749 | 108 | C > * |
| CA401360751 | 108 | C > W |
| CA294887113 | 109 | C > R |
| CA8814851 | 110 | Y > H |
| CA401360782 | 111 | I > F |
| CA401360786 | 111 | I > M |
| CA401360791 | 112 | P > A |
| CA8814852 | 114 | K > R |
| CA8814853 | 116 | G > V |
| CA401360860 | 118 | Q > P |
| CA8814856 | 121 | Q > * |
| CA401360891 | 121 | Q > P |
| CA8814858 | 122 | M > R |
| CA401360915 | 123 | G > R |
| CA401360930 | 124 | Q > E |
| CA401360928 | 124 | Q > K |
| CA401360953 | 126 | W > R |
| CA401360983 | 128 | F > V |
| CA8814862 | 130 | P > L |
| CA8814861 | 130 | P > S |
| CA401361020 | 131 | P > S |
| CA401361016 | 131 | P > T |
| CA401361042 | 133 | Y > N |
| CA8814865 | 134 | P > S |
| CA8814866 | 135 | S > R |
| CA294887228 | 139 | E > K |
| CA8814871 | 142 | S > R |
| CA401361288 | 143 | S > C |
| CA401361271 | 143 | S > P |
| CA401361370 | 146 | M > I |
| CA294887253 | 150 | A > G |
| CA294887248 | 150 | A > T |
| CA401361468 | 151 | T > A |
| CA401361469 | 151 | T > S |
| CA8814876 | 154 | R > C |
| CA8814877 | 154 | R > H |
| CA401361552 | 156 | T > P |
| CA401361565 | 156 | T > S |
| CA401361581 | 157 | P > S |
| CA401361593 | 158 | T > P |
| CA401361619 | 159 | F > L |
| CA294887341 | 161 | P > H |
| CA401361638 | 162 | K > R |
| CA8814880 | 163 | D > V |
| CA401361651 | 164 | I > F |
| CA8814883 | 168 | R > L |
| CA401361673 | 168 | R > P |
| CA8814882 | 168 | R > W |
| CA294887359 | 170 | D > G |
| CA401361709 | 171 | V > L |
| CA401361738 | 172 | M > I |
| CA401361729 | 172 | M > K |
| CA401361724 | 172 | M > V |
| CA401361746 | 173 | M > L |
| CA401361748 | 173 | M > V |
| CA8814886 | 174 | E > D |
| CA294887367 | 176 | E > K |
| CA294887368 | 177 | N > T |
| CA8814892 | 181 | F > L |
| CA8814894 | 182 | T > M |
| CA8814927 | 183 | I > M |
| CA401362038 | 184 | K > R |
| CA8814928 | 187 | A > G |
| CA294888081 | 188 | N > D |
| CA401362102 | 189 | R > K |
| CA8814929 | 190 | R > C |
| CA8814930 | 190 | R > L |
| CA294888098 | 191 | Y > H |
| CA8814931 | 191 | Y > N |
| CA401362134 | 192 | E > K |
| CA401362165 | 194 | P > L |
| CA401362170 | 195 | L > V |
| CA401362184 | 196 | E > Q |
| CA8814936 | 197 | T > N |
| CA8814937 | 198 | P > L |
| CA401362214 | 198 | P > R |
| CA294888175 | 198 | P > T |
| VAR_004286 | 199 | H > R |
| CA8814938 | 200 | V > D |
| CA401362236 | 200 | V > F |
| CA401362251 | 201 | H > Y |
| CA8814941 | 203 | R > Q |
| CA8814942 | 204 | A > T |
| CA401362293 | 205 | P > A |
| CA8814944 | 206 | S > C |
| CA401362303 | 206 | S > P |
| RCV001175569 | 208 | L > P |
| CA294888258 | 209 | Y > H |
| CA8814952 | 215 | E > K |
| CA401362453 | 218 | F > L |
| CA8814953 | 218 | F > S |
| CA401362473 | 220 | V > E |
| CA401362477 | 220 | V > G |
| CA401362502 | 222 | V > G |
| VAR_004287 | 223 | R > H |
| CA401362510 | 223 | R > P |
| CA401362504 | 223 | R > S |
| CA8814959 | 226 | L > P |
| CA198772 | 226 | L > V |
| CA401362586 | 228 | G > D |
| CA8814963 | 229 | R > H |
| CA401362611 | 230 | V > L |
| CA401362640 | 231 | L > P |
| CA8814993 | 234 | T > A |
| CA8814994 | 234 | T > M |
| CA8814996 | 235 | T > A |
| CA401363061 | 237 | A > E |
| CA401363074 | 238 | P > S |
| CA401363168 | 243 | D > N |
| CA401363204 | 244 | Q > H |
| CA294889597 | 245 | F > V |
| CA8815005 | 246 | L > F |
| CA401363297 | 250 | T > I |
| CA401363390 | 255 | Q > H |
| CA401363379 | 255 | Q > R |
| CA8815011 | 256 | Y > C |
| CA401363423 | 258 | T > I |
| CA401363464 | 262 | E > A |
| CA8815018 | 263 | H > R |
| CA294889693 | 264 | L > F |
| CA8815020 | 265 | S > R |
| CA401363512 | 268 | M > I |
| CA401363510 | 268 | M > I |
| CA401363521 | 270 | S > G |
| CA401363525 | 270 | S > N |
| CA401363558 | 272 | S > R |
| CA8815021 | 273 | W > * |
| CA8815022 | 274 | T > I |
| CA401363599 | 276 | I > F |
| RCV001090258 | 276 | I > V |
| CA401363615 | 277 | T > I |
| CA401363644 | 279 | W > C |
| CA8815026 | 281 | R > Q |
| CA401363673 | 282 | D > N |
| CA8815027 | 284 | A > T |
| CA8815030 | 285 | P > L |
| CA8815032 | 286 | T > M |
| CA401363807 | 287 | P > L |
| CA8815071 | 287 | P > S |
| CA401363883 | 293 | G > V |
| CA8815081 | 295 | H > N |
| CA401363965 | 300 | A > E |
| CA294889969 | 300 | A > V |
| CA8815087 | 305 | G > R |
| CA401364038 | 306 | S > A |
| VAR_068586 | 310 | V > G |
| CA294891320 | 310 | V > L |
| CA401364194 | 311 | F > L |
| CA8815093 | 312 | L > M |
| CA294891343 | 315 | S > N |
| CA401364267 | 317 | A > V |
| CA10606846 | 318 | M > L |
| CA294891694 | 320 | V > M |
| CA401364384 | 321 | V > A |
| CA294891725 | 321 | V > I |
| CA8815118 | 324 | P > Q |
| CA8815120 | 324 | P > R |
| CA294891758 | 325 | S > N |
| CA401364455 | 327 | A > G |
| CA8815125 | 332 | S > L |
| CA10604927 | 334 | G > C |
| CA294891791 | 336 | I > M |
| CA8815127 | 336 | I > N |
| CA294891797 | 338 | D > G |
| CA401364575 | 338 | D > Y |
| CA8815129 | 340 | Y > C |
| CA8815130 | 341 | I > V |
| CA401364670 | 346 | E > Q |
| CA401364694 | 347 | P > H |
| CA8815131 | 348 | K > R |
| CA401364711 | 349 | S > R |
| CA294891876 | 352 | Q > * |
| CA401364773 | 353 | Q > H |
| CA8815135 | 353 | Q > R |
| CA401364783 | 354 | Y > S |
| CA198782 | 357 | V > F |
| CA8815138 | 357 | V > I |
| CA401364892 | 360 | Y > S |
| CA294892083 | 361 | P > S |
| CA8815165 | 363 | M > R |
| RCV001194238 | 363 | M > V |
| CA8815166 | 364 | P > L |
| CA401364935 | 364 | P > S |
| CA8815168 | 365 | P > S |
| CA8815169 | 366 | Y > H |
| CA294892167 | 368 | G > C |
| CA8815170 | 368 | G > S |
| CA8815171 | 368 | G > V |
| CA401364969 | 370 | G > D |
| CA401364981 | 372 | H > D |
| CA401364985 | 372 | H > Q |
| CA401364995 | 374 | C > Y |
| CA401365013 | 377 | G > C |
| CA294892229 | 378 | Y > C |
| CA294892234 | 379 | S > F |
| CA294892232 | 379 | S > Y |
| CA401365044 | 382 | A > G |
| CA401365057 | 384 | T > I |
| CA401365055 | 384 | T > N |
| CA8815184 | 385 | R > H |
| CA401365065 | 386 | Q > R |
| CA401365070 | 387 | V > M |
| CA401365075 | 388 | V > L |
| CA8815187 | 388 | V > M |
| CA8815188 | 389 | E > K |
| CA8815191 | 391 | M > T |
| VAR_068593 | 391 | M > V |
| CA294892303 | 394 | A > T |
| CA8815193 | 395 | H > L |
| CA8815195 | 396 | F > L |
| CA8815194 | 396 | F > V |
| CA8815196 | 397 | P > R |
| CA8815198 | 398 | L > M |
| CA401365163 | 400 | V > A |
| CA401365192 | 404 | D > G |
| CA10604928 | 404 | D > V |
| CA8815235 | 407 | Y > C |
| CA234047 | 407 | Y > H |
| CA8815237 | 408 | M > L |
| CA401365214 | 408 | M > L |
| CA8815241 | 411 | R > Q |
| CA401365237 | 412 | R > G |
| CA294892536 | 412 | R > K |
| CA401365241 | 412 | R > S |
| CA294892544 | 415 | T > M |
| CA401365278 | 417 | N > K |
| CA401365292 | 419 | D > G |
| CA401365297 | 420 | G > D |
| CA294892550 | 420 | G > S |
| CA401365310 | 422 | R > L |
| CA8815245 | 422 | R > Q |
| CA401365311 | 423 | D > N |
| CA8815247 | 425 | P > Q |
| CA401365327 | 425 | P > T |
| CA401365338 | 427 | M > V |
| CA401365382 | 433 | Q > L |
| CA8815254 | 435 | G > S |
| CA401365395 | 435 | G > V |
| CA8815256 | 436 | R > Q |
| CA294892655 | 438 | Y > S |
| CA294892670 | 439 | M > I |
| CA294892660 | 439 | M > V |
| CA8815259 | 440 | M > T |
| CA8815287 | 444 | P > S |
| CA8815289 | 446 | I > L |
| CA8815292 | 449 | S > L |
| CA401366333 | 451 | P > S |
| CA8815295 | 452 | A > S |
| CA401366343 | 452 | A > T |
| CA401366358 | 453 | G > W |
| VAR_068597 | 458 | Y > C |
| CA8815299 | 459 | D > E |
| CA294894004 | 460 | E > D |
| CA401366468 | 461 | G > D |
| CA401366459 | 461 | G > S |
| CA8815302 | 464 | R > K |
| CA401366495 | 464 | R > S |
| CA401366490 | 464 | R > T |
| CA401366504 | 465 | G > E |
| CA401366496 | 465 | G > R |
| CA401366499 | 465 | G > W |
| CA8815305 | 466 | V > D |
| CA401366513 | 466 | V > G |
| CA8815304 | 466 | V > I |
| CA401366562 | 470 | N > S |
| CA8815311 | 471 | E > K |
| CA401366585 | 472 | T > P |
| CA401366591 | 472 | T > S |
| CA401366597 | 473 | G > C |
| CA401366599 | 473 | G > D |
| CA294894092 | 473 | G > R |
| CA294894100 | 474 | Q > L |
| CA8815314 | 475 | P > L |
| CA401366647 | 477 | I > M |
| CA294894127 | 478 | G > V |
| CA401366662 | 479 | K > E |
| CA401366767 | 480 | V > A |
| CA401366766 | 480 | V > E |
| CA8815347 | 480 | V > I |
| CA401366761 | 480 | V > L |
| CA401366772 | 481 | W > R |
| CA401366810 | 484 | S > A |
| CA401366817 | 484 | S > C |
| CA401366822 | 485 | T > A |
| CA401366836 | 486 | A > S |
| CA8815351 | 488 | P > A |
| CA401366926 | 493 | P > S |
| CA401366956 | 495 | A > V |
| CA198789 | 499 | W > R |
| CA401367013 | 500 | E > K |
| CA401367049 | 502 | M > R |
| CA401367077 | 505 | E > A |
| CA401367089 | 506 | F > L |
| CA401367111 | 509 | Q > L |
| CA294894901 | 510 | V > G |
| CA294894906 | 511 | P > L |
| CA401367148 | 515 | M > T |
| CA8815364 | 517 | I > V |
| CA401367191 | 519 | M > I |
| CA401367186 | 519 | M > V |
| CA401367197 | 520 | N > S |
| CA401367224 | 524 | N > K |
| CA401367222 | 524 | N > S |
| CA239454 | 528 | G > A |
| CA401367268 | 531 | D > N |
| CA294895023 | 532 | G > C |
| CA294895032 | 534 | P > L |
| CA401367292 | 534 | P > R |
| CA401367299 | 535 | N > K |
| CA401367297 | 535 | N > S |
| CA401367352 | 542 | P > L |
| CA8815393 | 542 | P > S |
| CA401367345 | 542 | P > T |
| CA401367360 | 543 | Y > C |
| CA915952253 | 544 | V > M |
| CA10606111 | 546 | G > V |
| CA8815423 | 547 | V > M |
| CA401368749 | 548 | V > A |
| CA8815424 | 548 | V > G |
| CA8815426 | 549 | G > W |
| CA294895748 | 550 | G > W |
| CA8815428 | 552 | L > I |
| CA401368824 | 553 | Q > K |
| CA8815430 | 553 | Q > R |
| CA401368854 | 554 | A > P |
| CA401368920 | 557 | I > S |
| CA401368917 | 557 | I > T |
| CA8815434 | 560 | S > F |
| CA401368990 | 562 | H > Q |
| CA401368994 | 563 | Q > R |
| CA294895795 | 567 | T > P |
| CA8815436 | 569 | Y > H |
| CA401369039 | 570 | N > Y |
| CA10604317 | 571 | L > P |
| CA401369052 | 572 | H > P |
| CA8815438 | 572 | H > Q |
| CA401369065 | 573 | N > K |
| CA401369071 | 574 | L > F |
| CA401369090 | 575 | Y > F |
| VAR_004300 | 576 | G > S |
| CA401369106 | 577 | L > P |
| CA401369103 | 577 | L > V |
| CA294895849 | 579 | E > Q |
| CA294895855 | 580 | A > V |
| CA8815443 | 581 | I > L |
| CA8815444 | 581 | I > M |
| CA294895864 | 582 | A > P |
| CA8815447 | 584 | H > D |
| CA401369180 | 584 | H > P |
| CA401369182 | 584 | H > R |
| VAR_068611 | 585 | R > K |
| CA294896266 | 586 | A > S |
| CA8815471 | 588 | V > M |
| CA8815474 | 592 | G > E |
| CA401369334 | 592 | G > R |
| CA8815475 | 593 | T > I |
| CA401369359 | 594 | R > C |
| CA401369395 | 597 | V > A |
| CA294896305 | 598 | I > V |
| CA401369410 | 599 | S > P |
| CA401369427 | 600 | R > P |
| CA401369447 | 602 | T > N |
| CA8815485 | 602 | T > S |
| CA401369491 | 606 | H > P |
| CA294896378 | 606 | H > Q |
| CA401369512 | 608 | R > G |
| CA294896401 | 608 | R > L |
| CA401369519 | 608 | R > P |
| CA401369524 | 609 | Y > D |
| CA8815490 | 609 | Y > H |
| CA401369553 | 611 | G > S |
| CA401369560 | 612 | H > P |
| CA8815494 | 612 | H > R |
| CA401369582 | 613 | W > C |
| CA401369573 | 613 | W > R |
| CA401369617 | 616 | D > E |
| CA294896447 | 616 | D > N |
| CA401369624 | 617 | V > A |
| CA294896458 | 617 | V > L |
| CA401369636 | 618 | W > G |
| CA401369676 | 621 | W > R |
| CA401369689 | 622 | E > K |
| CA401369691 | 622 | E > Q |
| CA8815502 | 624 | L > F |
| CA401369722 | 624 | L > V |
| CA8815504 | 625 | A > T |
| CA294896504 | 627 | S > F |
| CA8815506 | 628 | V > L |
| VAR_068621 | 629 | P > L |
| CA294896534 | 629 | P > S |
| CA294896808 | 630 | E > A |
| CA401369839 | 633 | Q > E |
| CA401369857 | 635 | N > S |
| CA401369862 | 636 | L > V |
| CA8815544 | 637 | L > M |
| CA401369872 | 638 | G > E |
| CA401369870 | 638 | G > R |
| CA401369877 | 639 | V > L |
| CA401369894 | 642 | V > I |
| CA294896832 | 643 | G > W |
| CA10605478 | 644 | A > P |
| CA8815550 | 644 | A > V |
| CA8815553 | 646 | V > I |
| CA401369920 | 647 | C > F |
| CA401369926 | 648 | G > V |
| CA294896886 | 651 | G > D |
| CA401369943 | 651 | G > V |
| CA8815556 | 653 | T > A |
| CA8815557 | 653 | T > N |
| CA401370002 | 661 | W > R |
| CA401370022 | 663 | Q > H |
| CA401370023 | 663 | Q > H |
| CA401370024 | 664 | L > V |
| CA401370036 | 666 | A > P |
| CA294896940 | 666 | A > V |
| CA401370051 | 668 | Y > C |
| CA401370050 | 668 | Y > S |
| CA401370056 | 669 | P > A |
| CA8815563 | 669 | P > R |
| CA401370055 | 669 | P > S |
| CA8815562 | 669 | P > T |
| CA401370068 | 671 | M > L |
| CA220392 | 671 | M > R |
| CA8815565 | 671 | M > T |
| CA8815564 | 671 | M > V |
| CA401370079 | 673 | N > T |
| CA401370094 | 674 | H > Y |
| CA401370115 | 675 | N > D |
| CA401370120 | 675 | N > S |
| CA8815567 | 675 | N > T |
| CA401370175 | 679 | S > R |
| CA401370294 | 682 | Q > K |
| CA401370340 | 684 | P > R |
| CA915952255 | 686 | S > K |
| CA401370371 | 686 | S > N |
| CA401370406 | 688 | S > N |
| CA8815603 | 688 | S > R |
| CA8815604 | 688 | S > R |
| CA8815605 | 689 | E > G |
| VAR_004309 | 689 | E > K |
| CA401370419 | 689 | E > Q |
| CA401370430 | 690 | P > R |
| CA401370433 | 691 | A > S |
| CA8815608 | 693 | Q > R |
| CA294897317 | 698 | A > D |
| CA401370485 | 698 | A > V |
| CA401370488 | 699 | L > F |
| CA401370489 | 699 | L > P |
| CA294897331 | 700 | T > A |
| CA294897382 | 703 | Y > C |
| CA401370512 | 704 | A > S |
| CA401370525 | 706 | L > F |
| CA401370528 | 706 | L > P |
| CA294897393 | 707 | P > L |
| CA8815618 | 708 | H > L |
| CA8815615 | 708 | H > N |
| CA401370556 | 711 | T > I |
| CA401370559 | 712 | L > Q |
| CA8815622 | 713 | F > L |
| CA294897442 | 714 | H > R |
| CA401370585 | 716 | A > P |
| CA401370586 | 716 | A > S |
| CA8815630 | 719 | A > V |
| CA8815631 | 720 | G > E |
| CA401370625 | 723 | V > G |
| CA8815635 | 723 | V > M |
| CA401370630 | 724 | A > D |
| CA401370627 | 724 | A > T |
| CA401370639 | 726 | P > L |
| CA401370648 | 728 | F > I |
| CA401370659 | 729 | L > P |
| CA401324684 | 732 | P > S |
| CA8815654 | 732 | P > T |
| CA8815655 | 733 | K > R |
| CA401324707 | 735 | S > F |
| CA401324703 | 735 | S > T |
| CA401324719 | 737 | T > I |
| CA294856435 | 740 | V > L |
| CA10607034 | 741 | D > V |
| CA401324761 | 744 | L > P |
| CA401324773 | 747 | G > E |
| CA8815667 | 747 | G > R |
| CA294856500 | 747 | G > W |
| CA401324808 | 753 | T > A |
| CA294856528 | 753 | T > I |
| CA8815670 | 753 | T > N |
| CA401324824 | 756 | L > F |
| CA8815672 | 757 | Q > P |
| CA401324830 | 757 | Q > R |
| CA8815675 | 761 | A > G |
| CA401324880 | 765 | G > D |
| CA294856634 | 766 | Y > N |
| CA8815680 | 766 | Y > S |
| CA294856691 | 771 | T > I |
| CA294856710 | 774 | D > E |
| CA401324934 | 774 | D > H |
| CA8815713 | 778 | V > L |
| CA401324975 | 779 | P > Q |
| VAR_004313 | 780 | V > I |
| CA401324977 | 780 | V > L |
| CA8815715 | 780 | V > L |
| CA294857110 | 781 | E > K |
| CA294857130 | 782 | A > V |
| CA8815716 | 783 | L > F |
| CA401325035 | 789 | P > L |
| CA294857155 | 789 | P > Q |
| CA8815719 | 789 | P > R |
| CA294857158 | 790 | P > S |
| CA8815720 | 791 | A > S |
| CA8815721 | 791 | A > V |
| CA294857164 | 794 | R > C |
| CA234052 | 794 | R > G |
| CA10606072 | 795 | E > K |
| CA8815725 | 798 | I > V |
| CA401325138 | 806 | T > A |
| CA8815733 | 806 | T > P |
| CA401325151 | 807 | L > P |
| CA401325165 | 809 | A > P |
| CA8815740 | 810 | P > L |
| CA8815739 | 810 | P > S |
| CA401325221 | 814 | I > T |
| CA401325227 | 815 | N > D |
| VAR_004314 | 816 | V > I |
| CA401325283 | 817 | H > R |
| CA8815749 | 823 | I > L |
| CA401325517 | 823 | I > V |
| CA10604584 | 825 | P > R |
| RCV001091539 | 827 | Q > * |
| CA401325545 | 827 | Q > R |
| CA294857876 | 828 | G > S |
| CA8815773 | 829 | P > L |
| CA401325955 | 830 | G > R |
| CA401325967 | 831 | L > F |
| CA8815775 | 833 | T > I |
| CA401325992 | 834 | T > A |
| CA294857899 | 836 | S > P |
| CA401326041 | 839 | Q > * |
| CA294857971 | 840 | P > A |
| CA401326065 | 841 | M > I |
| CA294857973 | 841 | M > T |
| CA8815780 | 844 | A > D |
| CA401326085 | 844 | A > T |
| CA401326104 | 846 | A > T |
| CA401326131 | 848 | T > I |
| CA8815784 | 851 | G > A |
| CA8815786 | 851 | G > E |
| CA8815785 | 851 | G > V |
| CA8815788 | 854 | R > Q |
| CA401326195 | 855 | G > R |
| CA401326193 | 855 | G > R |
| CA401326213 | 857 | L > M |
| CA294858045 | 864 | S > N |
| CA401326319 | 866 | E > A |
| CA401326321 | 866 | E > D |
| CA401326364 | 871 | G > E |
| CA401326408 | 875 | Q > R |
| CA401326462 | 879 | L > P |
| CA8815798 | 879 | L > V |
| CA294858091 | 880 | A > S |
| CA294858106 | 880 | A > V |
| CA8815799 | 881 | R > G |
| CA401326485 | 881 | R > S |
| CA8815825 | 884 | T > M |
| CA8815828 | 886 | V > M |
| CA8815832 | 891 | R > C |
| CA8815833 | 891 | R > H |
| CA401326709 | 892 | V > L |
| CA8815834 | 892 | V > M |
| CA8815835 | 897 | A > G |
| CA401326806 | 897 | A > T |
| CA401326843 | 898 | G > D |
| CA8815836 | 901 | L > M |
| CA401326934 | 903 | K > E |
| CA401326957 | 904 | V > M |
| CA401326986 | 906 | V > G |
| RCV001091541 | 906 | V > I |
| CA401326996 | 907 | L > P |
| CA8815841 | 912 | A > V |
| CA401327122 | 914 | Q > P |
| CA8815849 | 918 | S > C |
| CA401327196 | 919 | N > H |
| CA401327211 | 919 | N > S |
| CA401327260 | 922 | P > L |
| VAR_004317 | 927 | T > I |
| CA401327420 | 930 | P > T |
| CA401327438 | 931 | D > G |
| CA8815884 | 935 | L > V |
| CA8815885 | 937 | I > V |
| CA401327760 | 940 | S > L |
| CA8815888 | 943 | M > T |
| CA401327939 | 947 | F > L |
| CA198803 | 949 | V > I |
| CA8815891 | 950 | S > N |
| CA401328038 | 952 | C > G |

(ii) Nucleotide Sequences: N-Terminal GAA Polypeptide Truncations and Nucleic Acid Sequences Encoding GAA Polypeptides and N-Terminal Truncations, with Endogenous or Heterologous Signal Peptides.

As disclosed herein, the nucleic acids encoding a GAA protein disclosed herein, e.g., SEQ ID NOS: 1-18 that are codon optimized GAA nucleic acid sequence, for example, which have been modified from the NCBI GAA sequence of NM_00152.5 to include, any one or more of (i) enhanced expression in vivo, (ii) reduce CpG islands or reduction or elimination of CG dinucleotides, (iii) to reduce the innate immune response, and (iv) to reduce or eliminate alternative reading frames (ARF), or open reading frames (ORF). Exemplary codon optimized GAA nucleic sequences encompassed for use in the methods and rAAV compositions as disclosed herein can be selected from any of: SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17 or SEQ ID NO: 18 as disclosed herein, or a nucleic acid sequence having at least 60%, or 70%, or 80%, 85% or 90% or 95%, or 98%, or 99% sequence identity to SEQ ID NOS: 1-18, where SEQ ID NO: 1-18 or variants of at least 80% sequence identity thereto encode GAA polypeptide, where amino acid at position 199 is R (199R); amino acid at position 233 is H (233H), and amino acid at position 780 is I (780I).

In some embodiments, codon optimized GAA nucleic sequences encompassed for use in the methods and rAAV compositions as disclosed herein can be selected from any of: SEQ ID NO: 3 or SEQ ID NO: 4, or a nucleic acid sequence having at least 60%, or 70%, or 80%, 85% or 90% or 95%, or 98%, or 99% sequence identity to SEQ ID NO: 3 or SEQ ID NO: 4.

In some embodiments, the rAAV genome useful in the methods to treat Pompe Disease as disclosed herein comprises a heterologous nucleic acid sequence encoding a signal peptide (SP) fused in frame to the 5′ terminus of a GAA nucleic acid sequence that encodes a GAA polypeptide or N-terminal truncated GAA polypeptide, as disclosed herein. For example, heterologous nucleic acid sequence encoding a signal peptide (SP) is fused in frame to the 5′ terminus of a GAA nucleic acid sequence that encodes the GAA polypeptide or N-terminal truncated GAA polypeptide, so that both polypeptides are expressed from the rAAV genome when the rAAV vector transduces a mammalian cell.

In some embodiments, the rAAV genome useful in the methods to treat Pompe Disease as disclosed herein comprises a heterologous nucleic acid sequence comprising SEQ ID NO: 3, or a portion of SEQ ID NO: 3, where expression of a portion of the nucleic acid of SEQ ID NO: 3 produces a functional hGAA protein, and where the functional hGAA protein can comprise a N-terminal deletion of SEQ ID NO: 1, or a N-terminal and C-terminal truncation of SEQ ID NO: 1 as disclosed herein.

In some embodiments, the rAAV genome useful in the methods to treat Pompe Disease as disclosed herein comprises a heterologous nucleic acid sequence comprising SEQ ID NO: 3, or a nucleotides 82-2859 of SEQ ID NO: 3 or a nucleic acid having at least 85% sequence identity thereto, where nucleotides 1-81 of SEQ ID NO: 3 (corresponding to SEQ ID NO: 53) which encodes for a codon optimized hGAA signal peptide are replaced with the nucleic acid encoding a heterologous signal peptide as disclosed herein, e.g., selected from any of SEQ ID NOS: 54-58, 67, 72 or 75, or a nucleotide sequence having at least 85%, or at least 90%, or at least 95%, or at least 97%, or at least 98%, or at least 99% sequence identity thereto.

In some embodiments, the rAAV genome useful in the methods to treat Pompe Disease as disclosed herein comprises a heterologous nucleic acid sequence that comprises one or more base pair deletions at the 5′ of SEQ ID NO: 3.

For example, in some embodiments, the heterologous nucleic acid sequence in the AAV genome can comprise nucleotides selected from any of: 82-2859 of SEQ ID NO: 3, 82-2859 bp of SEQ ID NO: 3, 103-2859 bp of SEQ ID NO: 3, 118-2859 of SEQ ID NO: 3, 148-2859 bp of SEQ ID NO: 3, 169-2859 bp of SEQ ID NO: 3, 199-2859 bp of SEQ ID NO: 3, 205-2859 bp of SEQ ID NO: 3, 208-2859 bp of SEQ ID NO: 3, 214-2859 bp of SEQ ID NO: 3, 220-2859 bp of SEQ ID NO: 3, 265-2859 bp of SEQ ID NO: 3, 2335-2859 bp of SEQ ID NO: 3, 2368-2859 bp of SEQ ID NO: 3, 2371-2859 bp of SEQ ID NO: 3, 2374-2859 bp of SEQ ID NO: 3, 2377-2859 bp of SEQ ID NO: 3, 2386-2859 bp of SEQ ID NO: 3 or a nucleic acid having at least 85% sequence identity thereto, and attached to the 5′ of said sequence, nucleotides 1-81 (corresponding to SEQ ID NO: 53 or nucleotide sequence having at least 85%, or at least 90%, or at least 95%, or at least 97%, or at least 98%, or at least 99% sequence identity thereto) of SEQ ID NO: 3, which encodes for a codon optimized hGAA signal peptide.

In an alternative embodiment, the heterologous nucleic acid sequence in the AAV genome can comprise nucleotides selected from any of: 82-2859 of SEQ ID NO: 3, 82-2859 bp of SEQ ID NO: 3, 103-2859 bp of SEQ ID NO: 3, 118-2859 of SEQ ID NO: 3, 148-2859 bp of SEQ ID NO: 3, 169-2859 bp of SEQ ID NO: 3, 199-2859 bp of SEQ ID NO: 3, 205-2859 bp of SEQ ID NO: 3, 208-2859 bp of SEQ ID NO: 3, 214-2859 bp of SEQ ID NO: 3, 220-2859 bp of SEQ ID NO: 3, 265-2859 bp of SEQ ID NO: 3, 2335-2859 bp of SEQ ID NO: 3, 2368-2859 bp of SEQ ID NO: 3, 2371-2859 bp of SEQ ID NO: 3, 2374-2859 bp of SEQ ID NO: 3, 2377-2859 bp of SEQ ID NO: 3, 2386-2859 bp of SEQ ID NO: 3 or a nucleic acid having at least 85% sequence identity thereto, where nucleotides 1-81 (corresponding to SEQ ID NO: 53) of SEQ ID NO: 3, which encodes for a codon optimized hGAA signal peptide, are replaced with the nucleic acid encoding a heterologous signal peptide as disclosed herein, e.g., selected from any of SEQ ID NOS: 54-58, 67, 72 or 75, or a nucleotide sequence having at least 85%, or at least 90%, or at least 95%, or at least 97%, or at least 98%, or at least 99% sequence identity thereto.

The truncated GAA can be wildtype or codon optimized. For example, exemplary 5′ deletions of SEQ ID NO: 3 are disclosed in Table 1 herein, which encode for N-terminal truncated GAA polypeptide. In some embodiments, the 5′ of the 5′ deletions of SEQ ID NO: 3 can be attached to the 3′ of a nucleic acid encoding a signal peptide as disclosed herein, e.g., any signal peptide selected from any of SEQ ID NOS: 53-58, 67, 72 or 75, or a nucleotide sequence having at least 85%, or at least 90%, or at least 95%, or at least 97%, or at least 98%, or at least 99% sequence identity thereto.

TABLE 1
Table of exemplary N-terminal truncations of GAA polypeptide of SEQ ID
NO: 1, and the 5′ deletions of nucleic acid sequence of SEQ ID NO: 3.

| | | Corresponding nucleotides of |
|---|---|---|
| | Corresponding amino acid | coGAA nucleic acid of SEQ ID |
| N-terminal GAA truncation | residues of SEQ ID NO: 1 | NO: 3 (Seq100) |
| Signal peptide (which can be | residues 1-27 of SEQ ID NO: 1 | Nucleotides 1-81 of SEQ ID |
| removed and replaced with a | MGVRHPPCSHRLLAVCALVSL | NO: 3 |
| heterologous signal peptide | ATAALL (SEQ ID NO: 59) | ATGGGGGTGAGGCACCCTCC [symbol] |
| as disclosed herein) | | TGTTCTCACAGGCTGCTGGCT |
| | | GTGTGTGCTCTGGTGTCTCTG [symbol] |
| | | CCACTGCTGCCCTGCTG (SEQ |
| | | ID NO: 53) |
| hGAA polypeptide starting at | residues 28-952 of | 82-2859 bp of SEQ ID NO: 3 |
| residue 28 (removal of GAA | SEQ ID NO: 1 | |
| signal peptide) | | |
| hGAA polypeptide starting at | residues 35-952 of | 103-2859 bp of SEQ ID NO: 3 |
| residue 35 (A1-34 of SEQ ID | SEQ ID NO: 1 | |
| NO: 1) | | |
| hGAA polypeptide starting at | residues 40-952 of | 118-2859 of SEQ ID NO: 3 |
| residue 40 (A1-39 of SEQ ID | SEQ ID NO: 1 | |
| NO: 1) | | |
| hGAA polypeptide starting at | residues 50-952 of | 148-2859 bp of SEQ ID NO: 3 |
| residue 50 (A1-49 of SEQ ID | SEQ ID NO: 1 | |
| NO: 1) | | |
| hGAA polypeptide starting at | residues 57-952 of | 169-2859 bp of SEQ ID NO: 3 |
| residue 57 (A1-56 of SEQ ID | SEQ ID NO: 1 | |
| NO: 1) | | |
| hGAA polypeptide starting at | residues 68-952 of | 199-2859 bp of SEQ ID NO: 3 |
| residue 68 (A1-67 of SEQ ID | SEQ ID NO: 1 | |
| NO: 1) | | |
| hGAA polypeptide starting at | residues 69-952 of | 205-2859 bp of SEQ ID NO: 3 |
| residue 69 (A1-68 of SEQ ID | SEQ ID NO: 1 | |
| NO: 1) | | |
| hGAA polypeptide starting at | residues 70-952 of | 208-2859 bp of SEQ ID NO: 3 |
| residue 70 (A1-69 of SEQ ID | SEQ ID NO: 1 | |
| NO: 1) | | |
| hGAA polypeptide starting at | residues 72-952 of | 214-2859 bp of SEQ ID NO: 3 |
| residue 72 (A1-71 of SEQ ID | SEQ ID NO: 1 | |
| NO: 1) | | |
| hGAA polypeptide starting at | residues 74-952 of | 220-2859 bp of SEQ ID NO: 3 |
| residue 74 (A1-73 of SEQ ID | SEQ ID NO: 1 | |
| NO: 1) | | |
| hGAA polypeptide starting at | residues 89-952 of | 265-2859 bp of SEQ ID NO: 3 |
| residue 89 (A1-88 of SEQ ID | SEQ ID NO: 1 | |
| NO: 1) | | |
| hGAA polypeptide starting at | residues 779-952 of | 2335-2859 bp of SEQ ID NO: 3 |
| residue 779 (A1-778 of SEQ ID | SEQ ID NO: 1 | |
| NO: 1) | | |
| hGAA polypeptide starting at | residues 790-952 of | 2368-2859 bp of SEQ ID NO: 3 |
| residue 790 (A1-789 of SEQ ID | SEQ ID NO: 1 | |
| NO: 1) | | |
| hGAA polypeptide starting at | residues 791-952 of | 2371-2859 bp of SEQ ID NO: 3 |
| residue 791 (A1-790 of SEQ ID | SEQ ID NO: 1 | |
| NO: 1) | | |
| hGAA polypeptide starting at | residues 792-952 of | 2374-2859 bp of SEQ ID NO: 3 |
| residue 792 (A1-791 of SEQ ID | SEQ ID NO: 1 | |
| NO: 1) | | |
| hGAA polypeptide starting at | residues 793-952 of | 2377-2859 bp of SEQ ID NO: 3 |
| residue 793 (A1-792 of SEQ ID | SEQ ID NO: 1 | |
| NO: 1 of SEQ ID NO: 1) | | |
| hGAA polypeptide starting at | residues 796-952 of | 2386-2859 bp of SEQ ID NO: 3 |
| residue 796 (A1-795 of SEQ ID | SEQ ID NO: 1 | |
| NO: 1) | | |

[symbol]

indicates data missing or illegible when filed

In some embodiments, the rAAV genome useful in the methods to treat Pompe Disease as disclosed herein comprises a heterologous nucleic acid sequence that comprises nucleotides 103-2859 of SEQ ID NO: 3, where attached to the 5′ of said sequence, there is nucleic acid encoding a wildtype or codon optimized hGAA signal peptide (i.e., a nucleic acid comprising SEQ ID NO: 53), or fragment thereof, or a nucleic acid sequence encoding a heterologous signal peptide as disclosed herein, e.g., a nucleic acid sequence selected from any of SEQ ID NOS: 54-58, 67, 72 or 75, or a nucleotide sequence having at least 85%, or at least 90%, or at least 95%, or at least 97%, or at least 98%, or at least 99% sequence identity thereto.

In some embodiments, the rAAV genome useful in the methods to treat Pompe Disease as disclosed herein comprises a heterologous nucleic acid sequence that comprises nucleotides 118-2859 of SEQ ID NO: 3, where attached to the 5′ of said sequence, there is nucleic acid encoding a wildtype or codon optimized hGAA signal peptide (i.e., a nucleic acid comprising SEQ ID NO: 53) or a nucleic acid sequence encoding a heterologous signal peptide as disclosed herein, e.g., a nucleic acid sequence selected from any of SEQ ID NOS: 54-58, 67, 72 or 75, or a nucleotide sequence having at least 85%, or at least 90%, or at least 95%, or at least 97%, or at least 98%, or at least 99% sequence identity thereto.

In some embodiments, the rAAV genome useful in the methods to treat Pompe Disease as disclosed herein comprises a heterologous nucleic acid sequence that comprises nucleotides 148-2859 of SEQ ID NO: 3, where attached to the 5′ of said sequence, there is nucleic acid encoding a wildtype or codon optimized hGAA signal peptide (i.e., a nucleic acid comprising SEQ ID NO: 53) or a nucleic acid sequence encoding a heterologous signal peptide as disclosed herein, e.g., a nucleic acid sequence selected from any of SEQ ID NOS: 54-58, 67, 72 or 75, or a nucleotide sequence having at least 85%, or at least 90%, or at least 95%, or at least 97%, or at least 98%, or at least 99% sequence identity thereto.

In some embodiments, the rAAV genome useful in the methods to treat Pompe Disease as disclosed herein comprises a heterologous nucleic acid sequence that comprises nucleotides 169-2859 of SEQ ID NO: 3, attached to the 5′ of said sequence, there is nucleic acid encoding, e.g., a codon optimized hGAA signal peptide (i.e., a nucleic acid comprising SEQ ID NO: 53) or a nucleic acid sequence encoding a heterologous signal peptide as disclosed herein, e.g., a nucleic acid sequence selected from any of SEQ ID NOS: 54-58, 67, 72 or 75, or a nucleotide sequence having at least 85%, or at least 90%, or at least 95%, or at least 97%, or at least 98%, or at least 99% sequence identity thereto.

In some embodiments, the rAAV genome useful in the methods to treat Pompe Disease as disclosed herein comprises a heterologous nucleic acid sequence that comprises nucleotides 199-2859 of SEQ ID NO: 3, where attached to the 5′ of said sequence, there is nucleic acid encoding a wildtype or codon optimized hGAA signal peptide (i.e., a nucleic acid comprising SEQ ID NO: 53), or fragment thereof and/or a nucleic acid sequence encoding a heterologous signal peptide as disclosed herein, e.g., a nucleic acid sequence selected from any of SEQ ID NOS: 54-58, 67, 72 or 75, or a nucleotide sequence having at least 85%, or at least 90%, or at least 95%, or at least 97%, or at least 98%, or at least 99% sequence identity thereto.

In some embodiments, the rAAV genome useful in the methods to treat Pompe Disease as disclosed herein comprises a heterologous nucleic acid sequence that comprises nucleotides 205-2859 or nucleotides 208-2859, or nucleotides 214-2859 of SEQ ID NO: 3, where attached to the 5′ of said sequence, there is nucleic acid encoding a wild-type or codon optimized hGAA signal peptide (i.e., a nucleic acid comprising SEQ ID NO: 53) and/or a nucleic acid sequence encoding a heterologous signal peptide as disclosed herein, e.g., a nucleic acid sequence selected from any of SEQ ID NOS: 54-58, 67, 72 or 75, or a nucleotide sequence having at least 85%, or at least 90%, or at least 95%, or at least 97%, or at least 98%, or at least 99% sequence identity thereto.

In some embodiments, the rAAV genome useful in the methods to treat Pompe Disease as disclosed herein comprises a heterologous nucleic acid sequence that comprises nucleotides 220-2859 or nucleotides 208-2859, or nucleotides 214-2859 of SEQ ID NO: 3, where attached to the 5′ of said sequence, there is nucleic acid encoding a wild-type or codon optimized hGAA signal peptide (i.e., a nucleic acid comprising SEQ ID NO: 53) and/or a nucleic acid sequence encoding a heterologous signal peptide as disclosed herein, e.g., a nucleic acid sequence selected from any of SEQ ID NOS: 54-58, 67, 72 or 75, or a nucleotide sequence having at least 85%, or at least 90%, or at least 95%, or at least 97%, or at least 98%, or at least 99% sequence identity thereto.

In some embodiments, the rAAV genome useful in the methods to treat Pompe Disease as disclosed herein comprises a heterologous nucleic acid sequence that comprises any of: nucleotides 265-2859 of SEQ ID NO: 3, or nucleotides 2335-2859 of SEQ ID NO: 3, or nucleotides 2368-2859 of SEQ ID NO: 3, or nucleotides 2371-2859 bp of SEQ ID NO: 3, where attached to the 5′ of said sequence, there is nucleic acid encoding a wildtype or codon optimized hGAA signal peptide (i.e., a nucleic acid comprising SEQ ID NO: 53), or fragment thereof, and/or a nucleic acid sequence encoding a heterologous signal peptide as disclosed herein, e.g., a nucleic acid sequence selected from any of SEQ ID NOS: 54-58, 67, 72 or 75, or a nucleotide sequence having at least 85%, or at least 90%, or at least 95%, or at least 97%, or at least 98%, or at least 99% sequence identity thereto.

In some embodiments, the rAAV genome useful in the methods to treat Pompe Disease as disclosed herein comprises a heterologous nucleic acid sequence that encodes a signal sequence as disclosed herein, and a GAA polypeptide or a N-terminal truncated GAA polypeptide, where the GAA polypeptide begins at amino acid residues selected from any of: 28, 35, 40, 50, 57, 57, 68, 69, 70, 72, 74, 89, 779, 790, 791, 792, 793 or 796, and optionally, where the GAA polypeptide also has a C-terminal deletion of at least about 10, or about 20, or about 30, or about 40, or about 50, or about 60, or about 70, or about 80, or about 90, or about 100, or about 110, or more than 110 amino acid residues from the C-terminus of SEQ ID NO: 1. In some embodiment, the rAAV genome useful in the methods to treat Pompe Disease as disclosed herein comprises a heterologous nucleic acid sequence that encodes a signal sequence as disclosed herein, and a GAA polypeptide or a N-terminal truncated GAA polypeptide, where the GAA polypeptide begins at amino acid residues selected from any of: 28, 35, 40, 50, 57, 57, 68, 69, 70, 72, 74, 89, 779, 790, 791, 792, 793 or 796, and where the C-terminal of the GAA polypeptide occurs at any residue after amino acid residue 500, 600, 700, 800, 842, 852, 862, 875, 885, 895, 900, 905, 915, 920, 925, 930, 935, 940, 945, 950, 951 of SEQ ID NO. 1.

In some embodiments, the rAAV genome useful in the methods to treat Pompe Disease as disclosed herein comprises a heterologous nucleic acid sequence that (i) encodes a wildtype or codon optimized hGAA signal peptide (i.e., a nucleic acid comprising SEQ ID NO: 53), or fragment thereof and/or a nucleic acid sequence encoding a heterologous signal peptide as disclosed herein, e.g., a nucleic acid sequence selected from any of SEQ ID NOS: 54-58, 67, 72 or 75, or a nucleotide sequence having at least 85%, or at least 90%, or at least 95%, or at least 97%, or at least 98%, or at least 99% sequence identity to SEQ ID NOS 53-58, 67, 72 or 65, and (ii) encodes a GAA polypeptide, the GAA polypeptide encoded by a nucleic acid sequence beginning at base pairs selected from any of: 82, 103, 118, 148, 169, 199, 205, 208, 214, 220, 265 of SEQ ID NO: 3, and ending at base pair selected from any of: 2553 (i.e., deletion of 100 C-terminal residues, ends at 851aa), 2583 (i.e., deletion of 90 C-terminal residues, ends at 861aa), 2613 (i.e., deletion of 80 C-terminal residues, ends at 871aa), 2643 (i.e., deletion of 70 C-terminal residues, ends at 871aa), 2673 (i.e., deletion of 60 C-terminal residues, ends at 881aa), 2703 (i.e., deletion of 50 C-terminal residues, ends at 891aa), 2733 (i.e., deletion of 40 C-terminal residues, ends at 901aa), 2763 (i.e., deletion of 30 C-terminal residues, ends at 911aa), 2823 (i.e., deletion of 20 C-terminal residues, ends at 921aa), 2775 (i.e., deletion of 27 C-terminal residues, ends at 925aa), 2826 (i.e., deletion of 10 C-terminal residues, ends at 942aa) of SEQ ID NO: 3, or ending anywhere between base pairs 2553-2859 of SEQ ID NO: 3.

### B. Signal Peptide (SP)

The native GAA signal peptide is not cleaved in the ER thereby causing native GAA polypeptide to be membrane bound in the ER (Tsuji et al. (1987) Biochem. Int. 15(5):945-952). Disruption of the membrane association of GAA can be accomplished by replacing the endogenous GAA signal peptide (and optionally adjacent sequences) with an alternate signal peptide for GAA.

Accordingly, in representative embodiments, the rAAV vector and rAAV genome useful in the methods to treat Pompe disease as disclosed herein further comprises a heterologous nucleic acid encoding a GAA polypeptide to be transferred to a target cell, attached to a heterologous nucleic acid sequence that encodes a heterologus signal peptide in the place of the endogenous GAA signal peptide. The heterologous nucleic acid encoding a GAA polypeptide, including N-terminal truncations of the GAA polypeptide, which is is operatively associated with the segment encoding the secretory signal peptide, such that upon transcription and translation a fusion polypeptide is produced containing the secretory signal sequence operably associated with (e.g., directing the secretion of) the GAA polypeptide or N-terminal truncated GAA polypeptide.

In some embodiments, the endogenous signal peptide of hGAA (i.e., amino acids 1-27 of SEQ ID NO: 1 (encoded by a codon optimized nucleic acid sequence corresponding to SEQ ID NO: 59)) is replaced with a heterologous signal peptide (also referred to herein as a “signal sequence” or “leader sequence”) of SEQ ID NO: 60 (201 IgG signal peptide), or an IL2 wild type signal peptide (SEQ ID NO: 61), modified IL2 signal peptide (SEQ ID NO: 62), A2M signal peptide (SEQ ID NO: 63), or PZP signal peptide (SEQ ID NO: 64), or artificial signal peptide (SEQ ID NO: 65), or cathpetsin L signal peptide (SEQ ID NO: 66) or signal peptides at least 90% sequence identity to SEQ ID NOS: 60-66.

In some embodiments, the AAV vector encodes a GAA polypeptide that comprises the endogenous GAA signal peptide (e.g., amino acids 1-27 of SEQ ID NO: 1 (also referred to as “innate GAA” or “cognate GAA” signal peptide). In some embodiments, the AAV vector encodes a GAA polypeptide that comprises the endogenous GAA signal peptide (e.g., amino acids 1-27 of SEQ ID NO: 1, or a portions thereof) and an additional heterologous (i.e., non native) signal sequence. In some embodiments, the GAA polypeptide or N-terminal GAA polypeptide disclosed herein that lacks the endogenous signal peptide of amino acids 1-27 of GAA of SEQ ID NO: 1 is fused to a heterolous signal peptide (also referred to as “secretory signal peptide”).

In a particular embodiment, the heterologous nucleic acid sequence encodes a GAA polypeptide comprising a signal peptide fused to the GAA polypeptide, wherein the signal peptide is a heterologous GAA polypeptide. In some embodiments, the heterologous nucleic acid encoding a GAA polypeptide fused to a heterologous (e.g., exogenous or non-GAA) signal peptide can further comprise, at the 5′ end, a nucleic acid sequence encoding a portion of the cognate (e.g., endogenous) GAA signal peptide, e.g., encoding at least 1-5, or at least 1-10, or at least 1-20, or at least about 1-23, or at least about 1-24, or at least about 1-25, or at least about 1-26, or the entire GAA signal peptide, e.g., or at least about 1-27 concecutive amino acids of the endogenous GAA signal peptide. Stated differently, in some embodiments, the heterologous nucleic acid sequence can comprise, in the 5′ to 3′ direction, a nucleic acid sequence encoding the entire GAA signal peptide of SEQ ID NO: 59 or a portion of the endogenous GAA signal peptide of SEQ ID NO: 59, a nucleic acid sequence encoding a heterologous signal peptide (e.g., non-GAA signal peptide) and a a nucleic acid sequence encoding a GAA polypeptide, such as the wild-type nucleic acid sequences, or a sequence encoding at least 1-3 amino acid variants, or a codon-optimized nucleic acid sequence encoding a GAA polypeptide, including N-terminal GAA truncations. For example, in some embodiments, the nucleic acid sequence encoding the GAA polypeptide encodes a N-terminal truncated GAA polypeptide, such as those disclosed in Table 1 herein.

In some embodiments, the order of the signal peptides are changed, for example, in some embodiments, the heterologous nucleic acid sequence can comprise, in the 5′ to 3′ direction, a nucleic acid sequence encoding a heterologous signal peptide as disclosed herein, the entire GAA signal peptide of SEQ ID NO: 59 or a portion of the endogenous GAA signal peptide of SEQ ID NO: 59 and a wildtype or codon-optimized nucleic acid sequence encoding a GAA polypeptide, including N-terminal GAA truncations.

In some embodiments, there can be a portion of the GAA polypeptide, e.g., any 1 amino acid, or 2 amino acids or more than 2 concecutive amino acids located in the region of amino acids 28-56 of SEQ ID NO: 1) located between the GAA signal peptide (or portion thereof), and the heterologous signal peptide and upstream (e.g., N-terminal) of a N-terminal truncated GAA polypeptide. For example, and without wishing to be bound by theory, in some embodiments, the heterologous nucleic acid sequence can comprise, in the 5′ to 3′ direction, (i) the entire full length GAA signal peptide or a portion thereof as disclosed herein, (ii) a portion of at the the GAA polypeptide, e.g., amino acids 28-35 of SEQ ID NO: 1, amino acid 28 of SEQ ID NO: 1, or amino acids 28-31 of SEQ ID NO: 1, (iii) a heterologous signal peptide, and (iv) a GAA polypeptide, e.g., a N-terminal truncated GAA polypeptide beginning at amino acid 57 of SEQ ID NO: 1. It is envisioned that the portion of at the the GAA polypeptide located between the GAA polypeptide and the heterologous signal peptide can be any length, e.g., at least 1, 2, 3, 4, 5, 6, 7, 8, 9, 10, 10-12, 12,-14, 14,-16, 16-18, 18-20, or more than 20 amino acids of residues 28-56 of SEQ ID NO: 1, and the N-terminal GAA polypetide does not need to start at the next or sequential amino acid of the earlier GAA polypeptide portion. Certain of the amino acids in this 28-56 amino acid region can cause cellular retention. In one embodiment, those amino acids are removed or replaced.

In some embodiments, the nucleic acid sequence that encodes a GAA-signal peptide encodes at least 1-5, or at least 1-10, or at least 1-20, or at least about 1-23, or at least about 1-24, or at least about 1-25, or at least about 1-26, or at least about 1-27 concecutive amino acids (i.e., the full GAA signal peptide sequence), or non-concecutive amino acids of the endogenous GAA signal peptide of SEQ ID NO: 59. In some embodiments, the nucleic acid sequence encoding a GAA-signal peptide encodes a GAA signal peptide that comprises at least one deletion of at least 1, or at least 2, or at least 3, or at least 4, or at least 5, or at least 6, or at least 7, or at least 8, or at least 9, or at least 10, or at least 11, or at least 12, or at least 13, or at least 14, or at least 15, or at least 16, or at least 17, or at least 18, or at least 19, or at least 20 amino acids of SEQ ID NO: 59, where the one or more deletions can be concecutive, or non-concecutive deletions.

In some embodiments, the nucleic acid sequence that encodes a GAA-signal peptide can comprise the entire nucleic acid of SEQ ID NO: 57 (encoding GAA signal peptide comprising amino acids 1-27 of SEQ ID NO: 59). In alternative embodiments, the nucleic acid sequence that encodes a GAA-signal peptide can comprise a portion of the nucleic acid sequence of SEQ ID NO: 53, e.g., portions of SEQ ID NO: 53 that are selected from concecutive bases of SEQ ID NO: 53 having the length of any of: 1-3 bp, 1-4 bp, 1-5 bp, 1-6 bp, 1-7 bp, 1-8 bp, 1-9 bp, 1-10 bp, 1-11 bp, 1-12 bp, 1- 13 bp, 1-14 bp, 1-15 bp, 1-16 bp, 1-17 bp, 1-18 bp, 1-19 bp, 1-20 bp, 1-21 bp, 1-22 bp, 1-23 bp, 1-24 bp, 1- 25 bp, 1-26 bp, 1-27 bp, 1-28 bp, 1-29 bp, 1-30 bp, 1-33 bp, 1-36 bp, 1-39 bp, 1-42 bp, 1-45 bp, 1-48 bp, 1- 51 bp, 1-54 bp, 1-57 bp, 1-60 bp, 1-63 bp, 1-66 bp, 1-69 bp, 1-72b, 1-75 bp and 1-78 bp in any region of the nucleic acid sequence of SEQ ID NO: 53. That is, using a 21 bp portion of SEQ ID NO: 53 as an exemplary example, the GAA signal peptide can comprise a nucleic acid that is a 21 bp portion of SEQ ID NO: 53, where the 21 bp can be any 21-consecutive base pairs of SEQ ID NO: 53. For example, a 1-21 bp portion beginning at base pair 1 of SEQ ID NO: 53 would encode for a GAA-signal peptide comprising amino acids 1-7 of SEQ ID NO: 59, whereas a 1-21 bp portion beginning at base pair 15 of SEQ ID NO: 53 would encode for a GAA-signal peptide comprising amino acids 5-12 of SEQ ID NO: 59. In some embodiments, a portion of the GAA signal peptide of SEQ ID NO: 59 is the N-terminal portion of SEQ ID NO: 59 (i.e., has a deletion of at least 1, or at least 2, or at least 3, or at least 4, or at least 5, or at least 6, or at least 7, or at least 8, or at least 9, or at least 10, or at least 11, or at least 12, or at least 13, or at least 14 or at least 15 amino acids, or more than 15 amino acids at the C-terminal of SEQ ID NO: 59).

In some embodiments, the nucleic acid encoding a heterologous signal peptide as disclosed herein can be inserted into the nucleic acid sequence encoding a GAA-signal peptide. The insertion can occur at any location in the 1-81 bp of SEQ ID NO: 53. In some embodiments, a nucleic acid sequence encoding a heterologous signal peptide as disclosed herein is inserted in a portion of the nucleic acid encoding a GAA-signal peptide, e.g., generating a chimeric signal peptide comprising, for example, a 5′ nucleic acid sequence encoding a portion of a GAA signal peptide (e.g., a portion of SEQ ID NO: 53, as discussed above) and attached to the 3′ of said sequence, a nucleic acid encoding a heterologous signal peptide as disclosed herein. In some embodiments, there can also be a nucleic acid encoding a N-terminal portion of the GAA polypeptide with a deletion of amino acids thereafter, e.g., encoding amino acids 28, 28-29, 28-30, 28-31, 28-32, 28-33 etc., of SEQ ID NO: 1 followed by a deletion of about 4-40 amino acids of SEQ ID NO:1. In some embodiments, a heterologous signal peptide can be inserted immediately following the N-terminal amino acids, e.g., after position 28, 29, 30, 31, 32 or 33.

In some embodiments of the compositions and methods described herein, the signal peptides serve a general purpose of assisting the secretion of the GAA polypeptide from the liver cells into the blood, where it can travel and be targeted to the lysosomes of mammalian cells, for example, human cardiac and skeletal muscle cells, as described herein. In some embodiments, a heterologous signal peptide is selected from any of: a AAT signal peptide, a fibronectin signal peptide (FN1), 201 signal peptide, wtIL2 signal peptide, mutIL2 signal peptide, A2M signal peptide, PZP signal peptide, or an active fragment of AAT, FN1, 201, wtIL2, mutIL2, A2M or PZP signal peptide having secretory signal activity.

In some embodiments, the signal peptide is heterologous to (i.e., foreign or exogenous to) the polypeptide of interest. For example, a heterologous signal peptide is a fibronectin secretory signal peptide, the polypeptide of interest is not fibronectin. In some embodiments, the signal peptide is selected from any of: FN1, 201 signal peptide, wtIL2 signal peptide, mutIL2 signal peptide, A2M signal peptide, PZP signal peptide having secretory signal activity. In alternative embodiments, the signal peptide is not heterologous to GAA, i.e., the signal peptide is the GAA signal peptide (i.e., residues 1-27 of SEQ ID NO: 1, which the endogenous GAA polypeptide).

In some embodiments, the endogenous GAA signal sequence of amino acids 1-27 of SEQ ID NO: 1 (i.e., MGVRHPPCSHRLLAVCALVSLATAALL, SEQ ID NO: 59) is replaced with a different signal peptide (leader peptide). For example, the endogenous signal peptide of GAA (SEQ ID NO: 59) can be replaced with any of: (i) an IgG1 signal peptide (referred to herein as a “201 signal peptide” or “201Ip” having an amino acid sequence of: MEFGLSWVFLVALLKGVQCE (SEQ ID NO: 60) encoded by nucleic acid sequence SEQ ID NO: 54, (ii) wtIL2 lp: MYRMQLLSCIALSLALVTNS (SEQ ID NO: 61) encoded by nucleic acid sequence SEQ ID NO: 55, or (iii) mutIL2 lp: MYRMQLLLLIALSLALVTNS (SEQ ID NO: 62) encoded by nucleic acid sequence SEQ ID NO: 56, (iv) A2M signal peptide MGKNKLLHPSLVLLLLVLLPTDA (SEQ ID NO: 63) encoded by nucleic acid sequence SEQ ID NO: 57, (iv) PZP signal peptide MRKDRLLHLCLVLLLILLSASDSNS (SEQ ID NO: 64) encoded by nucleic acid sequence SEQ ID NO: 58. In some embodiments, the heterologous signal peptide can be truncated.

In some embodiments, the endogenous GAA signal peptide (SEQ ID NO: 59) or a fragment or portion thereof remains present, and an additional signal peptide is added, e.g., any one or more of signal peptides AAT, FN1, 201 signal peptide, wtIL2 signal peptide, mutIL2 signal peptide, A2M signal peptide, PZP signal peptide, as disclosed herein. In some embodiments, the endogenous GAA signal peptide of amino acids 1-27 of SEQ ID NO: 1 (i.e., MGVRHPPCSHRLLAVCALVSLATAALL, SEQ ID NO: 59) is replaced with a different or heterologous signal peptide. For example, the endogenous signal peptide of GAA (SEQ ID NO: 59) can be replaced with any of the heterologous signal peptides selected from: (i) an IgG1 signal peptide (referred to herein as a “201 signal peptide” or “201 lp” having an amino acid sequence of: MEFGLSWVFLVALLKGVQCE (SEQ ID NO: 60) encoded by nucleic acid sequence SEQ ID NO: 54, (ii) wtIL2 lp: MYRMQLLSCIALSLALVTNS (SEQ ID NO: 61) encoded by nucleic acid sequence SEQ ID NO: 55, or (iii) mutIL2 lp: MYRMQLLLLIALSLALVTNS (SEQ ID NO: 62) encoded by nucleic acid sequence SEQ ID NO: 56, (iv) A2M signal peptide MGKNKLLHPSLVLLLLVLLPTDA (SEQ ID NO: 63) encoded by nucleic acid sequence SEQ ID NO: 57, (iv) PZP signal peptide MRKDRLLHLCLVLLLILLSASDSNS (SEQ ID NO: 64) encoded by nucleic acid sequence SEQ ID NO: 58.

In some embodiments, the nucleic acid sequences in the rAAV vector or rAAV genome is a sequence selected from SEQ ID NO: 470-515. In one embodiment, the nucleic acid sequences in the rAAV vector or rAAV genome comprises at least a portion of a sequence selected from SEQ ID NO: 470-515 (i.e., a sequence that is at least 10%, 20%, 30%, 40%, 50%, 60%, 70%, 80%, 90%, 95% or more of the sequence of SEQ ID NO: 470-515).

In some embodiments, exemplary nucleic acid sequences in the rAAV vector or rAAV genome as disclosed herein are shown in exemplary constructs provided herein below:
pP065, is a plasmid comprising in the 5′ to 3′ direction: [L-ITR]-[(LSP:Liver Specific Promoter)]-[5′UTR]-[1-28aa GAA-SP]-[GAA polypeptide starting at amino acid 57 of SEQ ID NO:1]-[3′UTR]-[hGH polyA signal and Terminator]-[RNA polymerase II transcriptional pause signal]-[R-ITR]. See, e.g., SEQ ID NO: 470.
pP066, is a plasmid comprising in the 5′ to 3′ direction: [L-ITR]-[(LSP:Liver Specific Promoter)]-[5′UTR]-[1-28aa GAA-SP]-[GAA polypeptide starting at amino acid 57 of SEQ ID NO:1]-[3′UTR]-[hGH polyA signal and Terminator]-[RNA polymerase II transcriptional pause signal]-[R-ITR]. See, e.g., SEQ ID NO: 471.
pP067, is a plasmid comprising in the 5′ to 3′ direction: [L-ITR]-[(LSP:Liver Specific Promoter)]-[5′UTR]-[aa1 from GAA-SP]-[28-31aa GAA]-[IL-2 signal sequence]-[GAA polypeptide starting at amino acid 57 of SEQ ID NO:1]-[3′UTR]-[hGH polyA signal and Terminator]-[RNA polymerase II transcriptional pause signal]-[R-ITR]. See, e.g., SEQ ID NO: 472.
pP068, is a plasmid comprising in the 5′ to 3′ direction: [L-ITR]-[(LSP:Liver Specific Promoter)]-[5′UTR]-[aa1 from GAA-SP]-[28-31aa GAA]-[201Ig]-[GAA polypeptide starting at amino acid 57 of SEQ ID NO:1]-[3′UTR]-[hGH polyA signal and Terminator]-[RNA polymerase II transcriptional pause signal]-[R-ITR]. See, e.g., SEQ ID NO: 473.
pP069, is a plasmid comprising in the 5′ to 3′ direction: [L-ITR]-[(LSP:Liver Specific Promoter)]-[5′UTR]-[aa1 from GAA-SP from seq100]-[28-31aa GAA from seq3]-[IL-2 signal sequence]-[GAA polypeptide starting at amino acid 57 of seq3]-[3′UTR]-[hGH polyA signal and Terminator]-[RNA polymerase II transcriptional pause signal]-[ITR-neighborhood]-[R-ITR]. See, e.g., SEQ ID NO: 474.
pP070, is a plasmid comprising in the 5′ to 3′ direction: [L-ITR]-[(LSP:Liver Specific Promoter)]-[5′UTR]-[aa1 from GAA-SP]-[28-31aa GAA]-[201Ig]-[GAA polypeptide starting at amino acid 57 of SEQ ID NO:1]-[3′UTR]-[hGH polyA signal and Terminator]-[RNA polymerase II transcriptional pause signal]-[R-ITR]. See, e.g., SEQ ID NO: 475.
pP071, is a plasmid comprising in the 5′ to 3′ direction: [L-ITR]-[(LSP:Liver Specific Promoter)]-[5′UTR]-[1-24aa GAA]-[IL-2 signal sequence]-[GAA polypeptide starting at amino acid 57 of SEQ ID NO:1]-[3′UTR]-[hGH polyA signal and Terminator]-[RNA polymerase II transcriptional pause signal]-[R-ITR]. See, e.g., SEQ ID NO: 476.
pP072, is a plasmid comprising in the 5′ to 3′ direction: [L-ITR]-[(LSP:Liver Specific Promoter)]-[5′UTR]-[1-24aa GAA]-[201Ig]-[GAA polypeptide starting at amino acid 57 of SEQ ID NO:1]-[3′UTR]-[hGH polyA signal and Terminator]-[RNA polymerase II transcriptional pause signal]-[R-ITR]. See, e.g., SEQ ID NO: 477.
pP073, is a plasmid comprising in the 5′ to 3′ direction: [L-ITR]-[(LSP:Liver Specific Promoter)]-[5′UTR]-[1-24aa GAA]-[IL-2 signal sequence]-[GAA polypeptide starting at amino acid 57 of SEQ ID NO:1]-[3′UTR]-[hGH polyA signal and Terminator]-[RNA polymerase II transcriptional pause signal]-[R-ITR]. See, e.g., SEQ ID NO: 478.
pP074, is a plasmid comprising in the 5′ to 3′ direction: [L-ITR]-[(LSP:Liver Specific Promoter)]-[5′UTR]-[1-24aa GAA]-[201Ig]-[GAA polypeptide starting at amino acid 57 of SEQ ID NO:1]-[3′UTR]-[hGH polyA signal and Terminator]-[RNA polymerase II transcriptional pause signal]-[R-ITR]. See, e.g., SEQ ID NO: 479.
pP075, is a plasmid comprising in the 5′ to 3′ direction: [L-ITR]-[(LSP:Liver Specific Promoter)]-[5′UTR]-[1-23aa GAA]-[IL-2 signal sequence]-[GAA polypeptide starting at amino acid 57 of SEQ ID NO:1]-[3′UTR]-[hGH polyA signal and Terminator]-[RNA polymerase II transcriptional pause signal]-[R-ITR]. See, e.g., SEQ ID NO: 480.
pP076, is a plasmid comprising in the 5′ to 3′ direction: [L-ITR]-[(LSP:Liver Specific Promoter)]-[5′UTR]-[1-23aa GAA]-[201Ig]-[GAA polypeptide starting at amino acid 57 of SEQ ID NO:1]-[3′UTR]-[hGH polyA signal and Terminator]-[RNA polymerase II transcriptional pause signal]-[R-ITR]. See, e.g., SEQ ID NO: 481.
pP077, is a plasmid comprising in the 5′ to 3′ direction: [L-ITR]-[(LSP:Liver Specific Promoter)]-[5′UTR]-[1-23aa GAA]-[IL-2 signal sequence]-[GAA polypeptide starting at amino acid 57 of SEQ ID NO:1]-[3′UTR]-[hGH polyA signal and Terminator]-[RNA polymerase II transcriptional pause signal [R-ITR]. See, e.g., SEQ ID NO: 482.
pP078, is a plasmid comprising in the 5′ to 3′ direction: [L-ITR]-[(LSP:Liver Specific Promoter)]-[5′UTR]-[1-23aa GAA]-[201Ig]-[GAA polypeptide starting at amino acid 57 of SEQ ID NO:1]-[3′UTR]-[hGH polyA signal and Terminator]-[RNA polymerase II transcriptional pause signal]-[R-ITR]. See, e.g., SEQ ID NO: 483.
pP079, is a plasmid comprising in the 5′ to 3′ direction: [L-ITR]-[(LSP:Liver Specific Promoter)]-[5′UTR]-[1-2aa GAA]-[IL-2 signal sequence (Met1 removed)]-[GAA polypeptide starting at amino acid 57 of SEQ ID NO:1]-[3′UTR]-[hGH polyA signal and Terminator]-[RNA polymerase II transcriptional pause signal]-[R-ITR]. See, e.g., SEQ ID NO: 484.
pP080, is a plasmid comprising in the 5′ to 3′ direction: [L-ITR]-[(LSP:Liver Specific Promoter)]-[5′UTR]-[1-2aa GAA]-[201Ig(Met1 removed)]-[GAA polypeptide starting at amino acid 57 of SEQ ID NO:1]-[3′UTR]-[hGH polyA signal and Terminator]-[RNA polymerase II transcriptional pause signal]-[R-ITR]. See, e.g., SEQ ID NO: 485.
pP081, is a plasmid comprising in the 5′ to 3′ direction: [L-ITR]-[(LSP:Liver Specific Promoter)]-[5′UTR]-[1-2aa GAA]-[IL-2 signal sequence(Met1 removed)]-[GAA polypeptide starting at amino acid 57 of SEQ ID NO:1]-[3′UTR]-[hGH polyA signal and Terminator]-[RNA polymerase II transcriptional pause signal]-[R-ITR]. See, e.g., SEQ ID NO: 486.
pP082, is a plasmid comprising in the 5′ to 3′ direction: [L-ITR]-[(LSP:Liver Specific Promoter)]-[5′UTR]-[1-2aa GAA]-[201Ig(Met1 removed)]-[GAA polypeptide starting at amino acid 57 of SEQ ID NO:1]-[3′UTR]-[hGH polyA signal and Terminator]-[RNA polymerase II transcriptional pause signal]-[R-ITR]. See, e.g., SEQ ID NO: 487.
pP083, is a plasmid comprising in the 5′ to 3′ direction: [L-ITR]-[(LSP:Liver Specific Promoter)]-[5′UTR]-[1-4aa GAA]-[IL-2 signal sequence(Met1 removed)]-[GAA polypeptide starting at amino acid 57 of SEQ ID NO:1]-[3′UTR]-[hGH polyA signal and Terminator]-[RNA polymerase II transcriptional pause signal]-[R-ITR]. See, e.g., SEQ ID NO: 488.
pP084, is a plasmid comprising in the 5′ to 3′ direction: [L-ITR]-[(LSP:Liver Specific Promoter)]-[5′UTR]-[1-4aa GAA]-[201Ig(Met1 removed)]-[GAA polypeptide starting at amino acid 57 of SEQ ID No:1]-[3′UTR]-[hGH polyA signal and Terminator]-[RNA polymerase II transcriptional pause signal]-[R-ITR]. See, e.g., SEQ ID NO: 489.
pP085, is a plasmid comprising in the 5′ to 3′ direction: [L-ITR]-[(LSP:Liver Specific Promoter)]-[5′UTR]-[1-4aa GAA]-[IL-2 signal sequence(Met1 removed)]-[GAA polypeptide starting at amino acid 57 of SEQ ID NO:1]-[3′UTR]-[hGH polyA signal and Terminator]-[RNA polymerase II transcriptional pause signal]-[R-ITR]. See, e.g., SEQ ID NO: 490.
pP086, is a plasmid comprising in the 5′ to 3′ direction: [L-ITR]-[(LSP:Liver Specific Promoter)]-[5′UTR]-[1-4aa GAA]-[201Ig(Met1 removed)]-[GAA polypeptide starting at amino acid 57 of SEQ ID No:1]-[3′UTR]-[hGH polyA signal and Terminator]-[RNA polymerase II transcriptional pause signal]-[R-ITR]. See, e.g., SEQ ID NO: 491.
pP087, is a plasmid comprising in the 5′ to 3′ direction: [L-ITR]-[(LSP:Liver Specific Promoter)]-[5′UTR]-[1-10aa GAA]-[IL-2 signal sequence(Met1 removed)]-[GAA polypeptide starting at amino acid 57 of SEQ ID NO:1]-[3′UTR]-[hGH polyA signal and Terminator]-[RNA polymerase II transcriptional pause signal]-[R-ITR]. See, e.g., SEQ ID NO: 492.
pP088, is a plasmid comprising in the 5′ to 3′ direction: [L-ITR]-[(LSP:Liver Specific Promoter)]-[5′UTR]-[1-10aa GAA]-[201Ig(Met1 removed)]-[GAA polypeptide starting at amino acid 57 of SEQ ID NO:1]-[3′UTR]-[hGH polyA signal and Terminator]-[RNA polymerase II transcriptional pause signal]-[R-ITR]. See, e.g., SEQ ID NO: 493.
pP089, is a plasmid comprising in the 5′ to 3′ direction: [L-ITR]-[(LSP:Liver Specific Promoter)]-[5′UTR]-[1-10aa GAA]-[IL-2 signal sequence(Met1 removed)]-[GAA polypeptide starting at amino acid 57 of SEQ ID NO:1]-[3′UTR]-[hGH polyA signal and Terminator]-[RNA polymerase II transcriptional pause signal]-[R-ITR]. See, e.g., SEQ ID NO: 494.
pP090, is a plasmid comprising in the 5′ to 3′ direction: [L-ITR]-[(LSP:Liver Specific Promoter)]-[5′UTR]-[1-10aa GAA]-[201Ig(Met1 removed)]-[GAA polypeptide starting at amino acid 57 of SEQ ID NO;1]-[3′UTR]-[hGH polyA signal and Terminator]-[RNA polymerase II transcriptional pause signal]-[R-ITR]. See, e.g., SEQ ID NO: 495.
pP091, is a plasmid comprising in the 5′ to 3′ direction: [L-ITR]-[(LSP:Liver Specific Promoter)]-[5′UTR]-[1-27aa GAA]-[IL-2 signal sequence(Met1 removed)]-[GAA polypeptide starting at amino acid 57 of SEQ ID NO:1]-[3′UTR]-[hGH polyA signal and Terminator]-[RNA polymerase II transcriptional pause signal]-[R-ITR]. See, e.g., SEQ ID NO: 496.
pP092, is a plasmid comprising in the 5′ to 3′ direction: [L-ITR]-[(LSP:Liver Specific Promoter)]-[5′UTR]-[1-27aa GAA]-[201Ig(Met1 removed)]-[GAA polypeptide starting at amino acid 57 of SEQ ID NO:1]-[3′UTR]-[hGH polyA signal and Terminator]-[RNA polymerase II transcriptional pause signal]-[R-ITR]. See, e.g., SEQ ID NO: 497.
pP093, is a plasmid comprising in the 5′ to 3′ direction: [L-ITR]-[(LSP:Liver Specific Promoter)]-[5′UTR]-[1-27aa GAA]-[IL-2 signal sequence(Met1 removed)]-[GAA polypeptide starting at amino acid 57 of SEQ ID NO:1]-[3′UTR]-[hGH polyA signal and Terminator]-[RNA polymerase II transcriptional pause signal]-[ITR-neighborhood]-[R-ITR]. See, e.g., SEQ ID NO: 498.
pP094, is a plasmid comprising in the 5′ to 3′ direction: [L-ITR]-[(LSP:Liver Specific Promoter)]-[5′UTR]-[1-27aa GAA]-[201Ig(Met1 removed)]-[GAA polypeptide starting at amino acid 57 of SEQ ID NO:1]-[3′UTR]-[hGH polyA signal and Terminator]-[RNA polymerase II transcriptional pause signal]-[R-ITR]. See, e.g., SEQ ID NO: 499.
pP098, is a plasmid comprising in the 5′ to 3′ direction: [L-ITR]-[(LSP:Liver Specific Promoter)]-[5′UTR]-[1-24aa GAA]-[GAA polypeptide starting at amino acid 57 of SEQ ID NO:1]-[3′UTR]-[hGH polyA signal and Terminator]-[RNA polymerase II transcriptional pause signal]-[R-ITR]. See, e.g., SEQ ID NO: 500.
pP099, is a plasmid comprising in the 5′ to 3′ direction: [L-ITR]-[(LSP:Liver Specific Promoter)]-[5′UTR]-[1-24aa GAA]-[GAA polypeptide starting at amino acid 57 of SEQ ID NO:1]-[3′UTR]-[hGH polyA signal and Terminator]-[RNA polymerase II transcriptional pause signal]-[R-ITR]. See, e.g., SEQ ID NO: 501.
pP110, is a plasmid comprising in the 5′ to 3′ direction: [ITR2]-[LSP]-[1-28aa GAA]-[GAA polypeptide starting at amino acid 57 of SEQ ID NO:1]-[3′UTR]-[hGH polyA]-[ITR2]. See, e.g., SEQ ID NO: 502.
pP111, is a plasmid comprising in the 5′ to 3′ direction: [ITR2]-[LSP]-[1-24aa GAA]-[GAA polypeptide starting at amino acid 57 of SEQ ID NO:1]-[3′UTR]-[hGH polyA]-[ITR2]. See, e.g., SEQ ID NO: 503.
pP112, is a plasmid comprising in the 5′ to 3′ direction: [ITR2]-[LSP]-[1-27aa GAA]-[201Ip(Met1 removed)]-[GAA polypeptide starting at amino acid 57 of SEQ ID NO:1]-[3′UTR]-[hGH polyA]-[ITR2]. See, e.g., SEQ ID NO: 504.
pP113, is a plasmid comprising in the 5′ to 3′ direction: [ITR2]-[LSP]-[1-24aa GAA]-[201lp]-[GAA polypeptide starting at amino acid 57 of SEQ ID NO:1]-[3′UTR]-[hGH polyA]-[ITR2]. See, e.g., SEQ ID NO: 505.
pP114, is a plasmid comprising in the 5′ to 3′ direction: [ITR2]-[LSP]-[1-2aa GAA]-[IL-2 signal sequence (Met1 removed)]-[GAA polypeptide starting at amino acid 57 of SEQ ID NO:1]-[3′UTR]-[hGH polyA]-[ITR2]. See, e.g., SEQ ID NO: 506.
pP150, is a plasmid comprising in the 5′ to 3′ direction: [ITR2]-[LSP]-[1-24aa GAA]-[201Ip]-[GAA polypeptide starting at amino acid 57 of SEQ ID NO:1]-[3′UTR]-[hGH polyA]-[ITR2]. See, e.g., SEQ ID NO: 507.
pP151, is a plasmid comprising in the 5′ to 3′ direction: [ITR2]-[LSP]-[5′UTR]-[1-24aa GAA]-[201Ip]-[GAA polypeptide starting at amino acid 57 of SEQ ID NO:1]-[3′UTR]-[hGH polyA]-[ITR2]. See, e.g., SEQ ID NO: 508.
pP152, is a plasmid comprising in the 5′ to 3′ direction: [ITR2]-[LSP]-[1-24aa GAA]-[201Ip]-[GAA polypeptide starting at amino acid 57 of SEQ ID NO:1]-[3′UTR]-[hGH polyA]-[ITR2]. See, e.g., SEQ ID NO: 509.
pP153, is a plasmid comprising in the 5′ to 3′ direction: [ITR2]-[LSP]-[5′UTR]-[1-24aa GAA]-[201Ip]-[GAA polypeptide starting at amino acid 57 of SEQ ID NO:1]-[3′UTR]-[hGH polyA]-[ITR2]. See, e.g., SEQ ID NO: 510.
pP155, is a plasmid comprising in the 5′ to 3′ direction: [L-ITR]-[fragment of P5]-[LSP]-[1-24aa GAA]-[201Ip]-[GAA polypeptide starting at amino acid 57 of Seq ID NO;1]-[3′UTR]-[hGH polyA]-[R-ITR]. See, e.g., SEQ ID NO: 511.
pP157, is a plasmid comprising in the 5′ to 3′ direction: [L-ITR]-[fragment of P5]-[LSP]-[1-28aa GAA from ACTUS]-[GAA polypeptide starting at amino acid 57 of SEQ ID NO:1]-[3′UTR]-[hGH polyA]-[R-ITR]. See, e.g., SEQ ID NO: 512.

In some embodiments, the exemplary constructs have codon optimized GAA, including codon optimized GAA signal peptide, or portion thereof, e.g., the GAA and/or GAA signal peptide is encoded by seq 100 (SEQ ID NO:3), or seq3 (SEQ ID NO:4), or a fragment thereof. In some embodiments of the invention, the GAA and/or GAA signal peptides, or portion thereof in the exemplary constructs are not codon optimized, e.g., the GAA and/or GAA signal peptide is encoded by SEQ ID NO:2 or fragment thereof.

In some embodiments, the exemplary constructs described herein can be in a plasmid DNA backbone, or in a close ended linear duplexed DNA backbone or a precursor plasmid of close ended linear duplexed DNA backbone.

In some embodiments, the exemplary constructs described herein comprise 5′UTRs as described in the instant application including but not limited to SEQ ID NO:40 or SEQ ID NO:41.

In some embodiments, the nucleic acid encoding GAA the invention including but not limited to exemplary constructs, have 130 bp ITRs. In some embodiments of the invention, the nucleic acid encoding GAA of the invention including but not limited to exemplary constructs, have 145 bp ITRs.

In some embodiments, exemplary nucleic acid sequences in the rAAV vector or rAAV genome as disclosed herein are shown in Table 10.

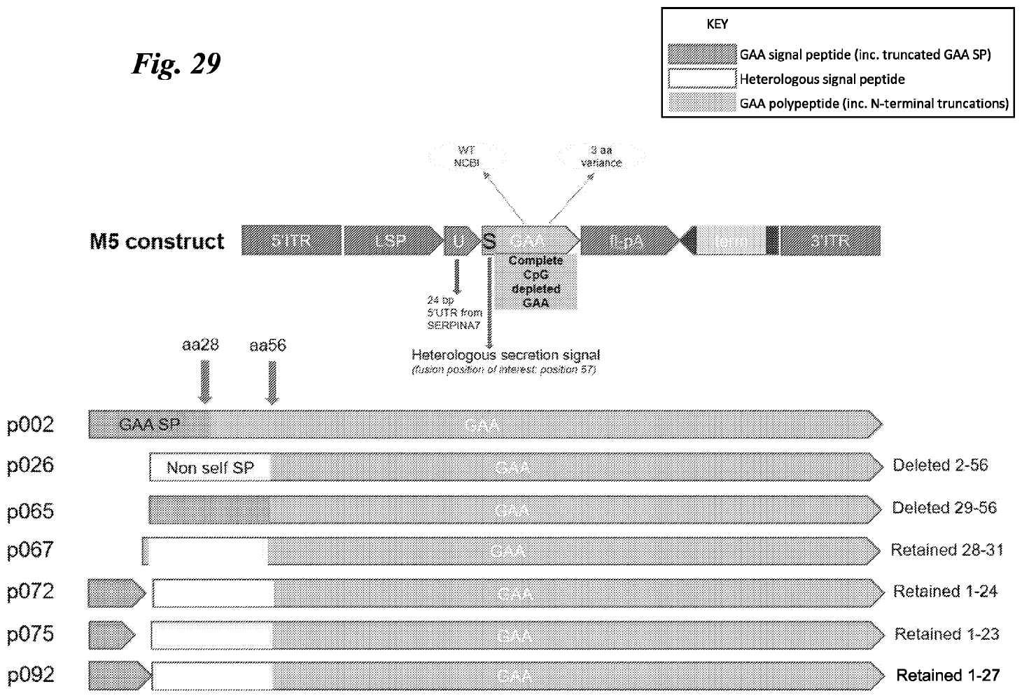

Table 10: Exemplary constructs encoding in a 5′ to 3′ direction a GAA-signal peptide, or portion thereof, a heterologous signal peptide and a GAA polypeptide (see, e.g., FIG. 29). In Table 10, a GAA polypeptide that begins at amino acid 57 of SEQ ID NO:1 is shown for exemplary purposes, however, any GAA polypeptide is envisoned, including any N-terminal truncation disclosed in Table 1 herein, as well as wild type GAA polypeptide, codon-optimized GAA polypeptides, ACTUS-101 GAA polypeptide, and N-terminal truncations thereof. (Hetero-SP, or SP refers to a heterologous signal peptide as disclosed herein, GAA-SP refers to the endogenous (cognate) signal peptide of GAA of SEQ ID NO: 59, or a portion thereof).

| | | | Amino acid | |
|---|---|---|---|---|
| Name | Description | Construct Summary | construct | Nucleic acid construct |
| P026 | Delta 2-56 | [1aa GAA-SP]- | [1aa of SEQ ID NO: | [1-3bp of SEQ ID NO: |
| | | [hetro-SP]-[delta1-56 | 59]-[hetro-SP]-[57- | 53]-[SP]-[169-2859bp |
| | | GAA/M5] | 952aa of SEQ ID | of SEQ ID NO: 3] |
| | | | NO: 1] | |
| P065 | Delta 29-56 | [1-27 aa GAA-SP]- | [1-27aa of SEQ ID | [1-81bp of SEQ ID |
| | | [delta 29-56 GAA] | NO: 59]-[28aa of | NO: 53]-[82-84 of |
| | | | SEQ ID NO: 1][57- | SEQ ID NO: 3][169- |
| | | | 952aa of SEQ ID | 2859bp of SEQ ID |
| | | | NO: 1] | NO: 3] |
| P067 | Retained 28-31 | [1-27 aa GAA-SP]- | [1-27aa of SEQ ID | [1-81bp of SEQ ID |
| | [28-31aa GAA]- | [28-31aa of GAA]- | NO: 59]-[28-31aa of | NO: 53]-[82-94bp of |
| | [SP]-[delta32-56]) | [hetero-SP]--[delta32- | SEQ ID NO: 1]- | SEQ ID NO: 3]-[SP]- |
| | | 56 GAA] | [hetro-SP]-[57- | [169-2859bp of SEQ |
| | | | 952aa of SEQ ID | ID NO: 3] |
| | | | NO: 1] | |
| P072 | Retained 1-24 [1- | [1-24 aa GAA-SP]- | [1-24aa of SEQ ID | [1-72bp of SEQ ID |
| | 24aa GAA-SP]- | [hetero-SP]--[delta25- | NO: 59]-[hetro-SP]- | NO: 53]-[SP]-[169- |
| | [SP]-[delta25-56]) | 56 GAA] | [57-952aa of SEQ | 2859bp of SEQ ID |
| | | | ID NO: 1] | NO: 3] |
| P075 | Retained 1-23 [1- | [1-23 aa GAA-SP]- | [1-23aa of SEQ ID | [1-69bp of SEQ ID |
| | 23aa GAA-SP]- | [hetero-SP]--[delta24- | NO: 59]-[hetro-SP]- | NO: 53]-[SP]-[169- |
| | [SP]-[delta24-56]) | 56 GAA] | [57-952aa of SEQ | 2859bp of SEQ ID |
| | | | ID NO: 1] | NO: 3] |
| P092 | Retained 1-27 [1- | [1-27 aa GAA-SP]- | [1-27aa of SEQ ID | 1-81bp of SEQ ID NO: |
| | 27aa GAA- | [28aa of GAA]- | NO: 59]-[hetro-SP]- | 53]-[82-84 of SEQ ID |
| | SP][28aa GAA]- | [hetero-SP]--[delta | [57-952aa of SEQ | NO: 3]-[SP]-[169- |
| | [SP]-[delta29-56]) | 29-56 GAA] | ID NO: 1] | 2859bp of SEQ ID |
| | | | | NO: 3] |

In one embodiment, the constructs described in Table 10 achieve the titers described in Table 11 following in vivo administration at 4 weeks-post administration.

TABLE 11
Summary of in vivo results 4 weeks post administration.

| | Serum | Serum | Heart | Heart | Liver |
|---|---|---|---|---|---|
| Group | GAA | 4MU | 4MU | glycogen | retention |
| GAA KO | 3.4e4 | 2.6e3 | 59.3 | 38.0 | 2.1e4 |
| 002/M4 | 2.0e5 | 4.5e3 | 65.0 | 30.7 | 2.8e6 |
| 026/M5 | 6.6e6 | 2.7e4 | 366.4 | 21.9 | 5.5e5 |
| 065 | 6.6e6 | 1.5e4 | 343.8 | 26.1 | 2.2e5 |
| 067 | 5.9e6 | 1.9e4 | 294.9 | 20.5 | 1.1e6 |
| 072 | 6.6e6 | 3.9e4 | 512.9 | 18.1 | 6.4e5 |
| 075 | 5.8e6 | 2.6e4 | 626.6 | 27.6 | 4.6e5 |
| 092 | 4.2e5 | 6.6e3 | 193.2 | 17.3 | 4.0e4 |
| WT mice | ND | ND | 482.1 | 20.6 | ND |

In Table 11, “Serum GAA” describes the level of GAA expression found in the serum of the injected mice 4 weeks post administration; “Serum 4MU” describes the level of GAA activity found in the serum of the injected mice 4 weeks post administration; “Heart 4MU” describes the level of GAA activity found in the heart of the injected mice 4 weeks post administration; “Heart glycogen” describes the level of glucose found in the heart of the injected mice 4 weeks post administration; and “Liver retention” describes the level of GAA expression found in the liver of the injected mice 4 weeks post administration.

In general, the GAA-signal peptide and/or heterologous signal peptide will be at the amino-terminus (N-terminus) of the GAA polypeptide (i.e., the nucleic acid segment encoding the signal peptide is 5′ to the heterologous nucleic acid encoding the GAA peptide in the rAAV vector or rAAV genome as disclosed herein). Alternatively, the signal peptide may be at the carboxyl-terminus or embedded within the GAA polypeptide, as long as the signal peptide is operatively associated therewith and directs secretion of the GAA polypeptide or GAA fusion polypeptide of interest (either with or without cleavage of the signal peptide from the GAA polypeptide) from the cell.

The signal peptide is operatively associated with the GAA polypeptide, including N-terminal truncated GAA polypeptides as disclosed in Table 1 herein, is targeted to the secretory pathway. Alternatively stated, the signal peptide is operatively associated with the GAA polypeptide such that the GAA-polypeptide is secreted from the cell at a higher level (i.e., a greater quantity) than in the absence of the secretory signal peptide. In general, typically at least about 20%, 30%, 40%, 50%, 70%, 80%, 85%, 90%, 95% or more of the GAA-polypeptide is secreted from the cell when a signal peptide is attached as compared to in the absence of the attachment of a secretory signal peptide. In other embodiments, essentially all of the detectable polypeptide (alone and/or in the form of the fusion polypeptide) is secreted from the cell.

By the phrase “secreted from the cell”, the polypeptide may be secreted into any compartment (e.g., fluid or space) outside of the cell including but not limited to: the interstitial space, blood, lymph, cerebrospinal fluid, kidney tubules, airway passages (e.g., alveoli, bronchioles, bronchia, nasal passages, etc.), the gastrointestinal tract (e.g., esophagus, stomach, small intestine, colon, etc.), vitreous fluid in the eye, and the cochlear endolymph, and the like.

Accordingly, in some embodiments, a AAV expressing GAA useful in the methods to treat Pompe Disease as disclosed herein comprises a 5′ ITR and 3′ ITR sequence, and located between the 5′ITR and the 3′ ITR, a liver specific promoter operatively linked to a heterologous nucleic acid encoding a secretory peptide and nucleic acid encoding an alpha-glucosidase (GAA) polypeptide (i.e., the heterologous nucleic acid encodes a GAA polypeptide or N-terminal GAA polypeptide comprising a signal peptide-GAA polypeptide).

In alternative embodiments, a AAV expressing GAA useful in the methods to treat Pompe Disease as disclosed herein comprises a 5′ ITR and 3′ ITR sequence, and located between the 5′ITR and the 3′ ITR, a promoter operatively linked to a heterologous nucleic acid encoding a secretory peptide and nucleic acid encoding an alpha-glucosidase (GAA) polypeptide.

Generally, secretory signal peptides are cleaved within the endoplasmic reticulum and, in some embodiments, the signal peptide is cleaved from the GAA polypeptide prior to secretion. It is not necessary, however, that the signal peptide is cleaved as long as secretion of the GAA polypeptide from the cell is enhanced and the GAA polypeptide is functional. Thus, in some embodiments, the signal peptide is partially or entirely retained.

In some embodiments, the rAAV genome, or an isolated nucleic acid as disclosed herein comprises a nucleic acid encoding a chimeric polypeptide comprising a GAA polypeptide operably linked to a secretory signal peptide, and the chimeric polypeptide is expressed and produced from a cell transduced with the rAAV vector and the GAA polypeptide is secreted from the cell. The GAA polypeptide can be secreted after cleavage of all or part of the secretory signal peptide. Alternatively, the GAA polypeptide can retain the signal peptide (i.e., the signal peptide is not cleaved). Thus, in this context, the “GAA polypeptide” can be a chimeric polypeptide comprising the secretory peptide.

Other signal peptide as encompassed for use in the methods and compositions as disclosed herein. For example, numerous secreted proteins and sequences that direct secretion from the cell are known in the art, are disclosed in U.S. Pat. No. 9,873,868, which is incorporated herein in its entirety by reference. Exemplary secreted proteins (and their secretory signals) include but are not limited to: erythropoietin, coagulation Factor IX, cystatin, lactotransferrin, plasma protease C1 inhibitor, apolipoproteins (e.g., APO A, C, E), MCP-1, α-2-HS-glycoprotein, α-1-microgolubilin, complement (e.g., C1Q, C3), vitronectin, lymphotoxin-α, azurocidin, VIP, metalloproteinase inhibitor 2, glypican-1, pancreatic hormone, clusterin, hepatocyte growth factor, insulin, α-1-antichymotrypsin, growth hormone, type IV collagenase, guanylin, properdin, proenkephalin A, inhibin β (e.g., A chain), prealbumin, angiocenin, lutropin (e.g., β chain), insulin-like growth factor binding protein 1 and 2, proactivator polypeptide, fibrinogen (e.g., β chain), gastric triacylglycerol lipase, midkine, neutrophil defensins 1, 2, and 3, α-1-antitrypsin, matrix gla-protein, α-tryptase, bile-salt-activated lipase, chymotrypsinogen B, elastin, IG lambda chain V region, platelet factor 4 variant, chromogranin A, WNT-1 proto-oncogene protein, oncostatin M, β-neoendorphin-dynorphin, von Willebrand factor, plasma serine protease inhibitor, serum amyloid A protein, nidogen, fibronectin, rennin, osteonectin, histatin 3, phospholipase A2, cartilage matrix Protein, GM-CSF, matrilysin, neuroendocrine protein 7B2, placental protein 11, gelsolin, M-CSF, transcobalamin I, lactase-phlorizin hydrolase, elastase 2B, pepsinogen A, MIP 1-β, prolactin, trypsinogen II, gastrin-releasing peptide II, atrial natriuretic factor, secreted alkaline phosphatase, pancreatic α-amylase, secretogranin I, β-casein, serotransferrin, tissue factor pathway inhibitor, follitropin β-chain, coagulation factor XII, growth hormone-releasing factor, prostate seminal plasma protein, interleukins (e.g., 2, 3, 4, 5, 9, 11), inhibin (e.g., alpha chain), angiotensinogen, thyroglobulin, IG heavy or light chains, plasminogen activator inhibitor-1, lysozyme C, plasminogen activator, antileukoproteinase 1, statherin, fibulin-1, isoform B, uromodulin, thyroxine-binding globulin, axonin-1, endometrial α-2 globulin, interferon (e.g., alpha, beta, gamma), β-2-microglobulin, procholecystokinin, progastricsin, prostatic acid phosphatase, bone sialoprotein II, colipase, Alzheimer's amyloid A4 protein, PDGF (e.g., A or B chain), coagulation factor V, triacylglycerol lipase, haptoglobuin-2, corticosteroid-binding globulin, triacylglycerol lipase, prorelaxin H2, follistatin 1 and 2, platelet glycoprotein IX, GCSF, VEGF, heparin cofactor II, antithrombin-III, leukemia inhibitory factor, interstitial collagenase, pleiotrophin, small inducible cytokine A1, melanin-concentrating hormone, angiotensin-converting enzyme, pancreatic trypsin inhibitor, coagulation factor VIII, α-fetoprotein, α-lactalbumin, senogelin II, kappa casein, glucagon, thyrotropin beta chain, transcobalamin II, thrombospondin 1, parathyroid hormone, vasopressin copeptin, tissue factor, motilin, MPIF-1, kininogen, neuroendocrine convertase 2, stem cell factor procollagen al chain, plasma kallikrein keratinocyte growth factor, as well as any other secreted hormone, growth factor, cytokine, enzyme, coagulation factor, milk protein, immunoglobulin chain, and the like.

In one embodiment, the secretory signal peptide is not a secretory signal peptide of α-1-antitrypsin (e.g., amino acids 1-24 of α-1-antitrypsin), chymotrypsinogen B2 (e.g., amino acids 1-20 of chymotrypsinogen B2), iduronate-2-sulphatase (e.g., amino acids 1-25 of iduronate-2-sulphatase), or protease C1 inhibitor (e.g., amino acids 1-23 of protease CI inhibitor).

In some embodiments, other secretory signal peptides encoded by the rAAV genome and in the rAAV vector as disclosed herein can be selected from, but are not limited to, the signal peptide sequences from prepro-cathepsin L (e.g., GenBank Accession Nos. KHRTL, NP_037288; NP_034114, AAB81616, AAA39984, P07154, CAA68691; the disclosures of which are incorporated by reference in their entireties herein) and prepro-alpha 2 type collagen (e.g., GenBank Accession Nos. CAA98969, CAA26320, CGHU2S, NP_000080, BAA25383, P08123; the disclosures of which are incorporated by reference in their entireties herein) as well as allelic variations, modifications and functional fragments thereof (as discussed above with respect to the fibronectin signal peptide sequence). Exemplary signal peptide sequences include for preprocathepsin L (Rattus norvegicus, MTPLLLLAVLCLGTALA [SEQ ID NO: 77]; Accession No. CAA68691) and for prepro-alpha 2 type collagen (Homo sapiens, MLSFVDTRTLLLLAVTLCLATC [SEQ ID NO: 78]; Accession No. CAA98969). Also encompassed are longer amino acid sequences comprising the full-length signal peptide sequence from preprocathepsin L and prepro-alpha 2 type collagen or functional fragments thereof (as discussed above with respect to the fibronectin signal peptide sequence).

In some embodiments, the signal peptide is derived in part or in whole from a secreted polypeptide that is produced by liver cells. In some embodiments, a signal peptide can further be in whole or in part synthetic or artificial. Synthetic or artificial secretory signal peptides are known in the art, see e.g., Barash et al., “Human signal peptide description by hidden Markov model and generation of a strong artificial signal peptide for secreted protein expression,” Biochem. Biophys. Res. Comm. 294:835-42 (2002); the disclosure of which is incorporated herein in its entirety. In particular embodiments, the signal peptide comprises, consists essentially of, or consists of the artificial secretory signal: MWWRLWWLLLLLLLLWPMVWA (SEQ ID NO: 65) or variations thereof having 1, 2, 3, 4, or 5 amino acid substitutions (optionally, conservative amino acid substitutions, conservative amino acid substitutions are known in the art).

Exemplary signal peptides for use in the methods and compositions as disclosed herein can be selected from any signal peptide disclosed in Table 2, or portions thereof or functional variants thereof. Exemplary signal peptides are Fibronectin (FN1), or AAT. In some embodiments of the methods and compositions disclosed herein, the rAAV vector composition comprises the nucleic acid encoding a secretory signal peptide, e.g., encoding a signal peptide selected from an AAT signal peptide (e.g., SEQ ID NO: 67), a fibronectin signal peptide (FN1) (e.g., SEQ ID NO: 68-71), an hIGF2 signal peptide (e.g., SEQ ID NO: 72) or an active fragment thereof having secretory signal activity, e.g., a nucleic acid encoding an amino acid sequence that has at least about 75%, or 80%, or 85%, or 90%, or 95%, or 98%, or 99% sequence identity to SEQ ID NOs: 67-72.

In some embodiments of the methods and compositions as disclosed herein, the nucleic acid encoding the signal peptide is selected from any of SEQ ID NO: 54-58, 67, 72-76 and 72, or a nucleic acid sequence at least about 75%, or 80%, or 85%, or 90%, or 95%, or 98%, or 99% sequence identity to any of SEQ ID NOs: 54-58, 67, 72-76 and 72.

### Fibronectin Secretory Signal Peptide:

In some embodiments, the signal peptide is a fibronectin secretory signal peptide or portions thereof or functional variants thereof, which term includes modifications of naturally occurring sequences (as described in more detail below).

In some embodiments, the signal peptide is a fibronectin signal peptide, e.g., a signal sequence of human fibronectin or a signal sequence from rat fibronectin. Fibronectin (FN1) signal sequences and modified FN1 signal peptides encompassed for use in the rAAV genome and rAAV vectors described herein are disclosed in U.S. Pat. No. 7,071,172, which is incorporated herein in its entirety by reference, and in Table 3 of provisional application 62/937,556, filed on Nov. 19, 2019 or International Application WO2021102107, which is incorporated herein in its reference. Examples of exemplary fibronectin signal peptide sequences include, but are not limited to those listed in Table 1 of U.S. Pat. No. 7,071,172, which is incorporated herein in its entirety by reference.

Table 2: Exemplary Fibronectin (FN1) secretory signal peptides

| Species | Signal peptide sequence | Nucleic acid sequence |
|---|---|---|
| H. Sapiens | MLRGPGPGLLLLAVQCLGTAV | ATG CTT AGG GGT CCG GGG CCC GGG CTG |
| | PSTGA (SEQ ID NO: 70) | CTG CTG CTG GCC GTC CAG TGC CTG GGG |
| | | ACA GCG GTG CCC TCC ACG GGA GCC |
| | | (SEQ ID NO: 75) |
| R. | MLRGPGPGRLLLLAVLCLGTSV | 5′- |
| Norvegicus | RCTETGKSKR (SEQ ID NO: 68) | ATGCTCAGGGGTCCGGGACCCGGGCGGCT |
| | | GCTGCTGCTAGCAGTCCTGTGCCTGGGGAC |
| | | ATCGGTGCGCTGCACCGAAACCGGGAAGA |
| | | GCAAGAGG-3 (SEQ ID NO: 73) |
| R. | MLRGPGPGRLLLLAVLCLGTSV | 5′-ATG CTC AGG GGT CCG GGA CCC GGG |
| Norvegicus | RCTETGKSKR ↑ LALQIV | CGG CTG CTG CTG CTA GCA GTC CTG TGC |
| | (SEQ ID NO: 69) | CTG GGG ACA TCG GTG CGC TGC |
| | | ACC GAA ACC GGG AAG AGC AAG AGG ↑ |
| | | CAG GCT CAG CAA ATC GTG-3′. (SEQ ID |
| | | NO: 74) (↑ denotes the cleavage site) |
| X. laevis | MRRGALTGLLLVLCLSVVLRA | ATG CGC CGG GGG GCC CTG ACC GGG CTG |
| | APSATSKKRR (SEQ ID NO: 71) | CTC CTG GTC CTG TGC CTG AGT GTT GTG |
| | | CTA CGT GCA GCC CCC TCT GCA ACA AGC |
| | | AAG AAG CGC AGG (SEQ ID NO: 76) |

### Peptidase Cleavage Sites

In some embodiments, one or more exogenous peptidase cleavage site may be inserted into the signal peptide-GAA polypeptide, e.g., between the signal peptide and the GAA polypeptide. In particular embodiments, an autoprotease (e.g., the foot and mouth disease virus 2A autoprotease) is inserted between the signal peptide and the GAA polypeptide. In other embodiments, a protease recognition site that can be controlled by addition of exogenous protease is employed (e.g., Lys-Arg recognition site for trypsin, the Lys-Arg recognition site of the Aspergillus KEX2-like protease, the recognition site for a metalloprotease, the recognition site for a serine protease, and the like). Modification of the GAA polypeptide to delete or inactivate native protease sites is encompassed herein and disclosed in U.S. Provisional Application 62,937,556, filed on Nov. 19, 2019 and International Application WO2021102107, which is incorporated herein in its reference.

### D. Spacer and Fusion Junction of the GAA Polypeptide

Where GAA is expressed with a heterologous signal peptide, the signal peptide can be fused directly to the GAA polypeptide or can be separated from the GAA polypeptide by a linker. An amino acid linker (also referred to herein as a “spacer”) incorporates one or more amino acids other than that appearing at that position in the natural protein. Spacers can be generally designed to be flexible or to interpose a structure, such as an a-helix, between the two protein moieties.

Accordingly, in some embodiments of the methods and compositions disclosed herein, a recombinant AAV vector comprises a heterologous nucleic acid sequence encoding an GAA polypeptide, wherein the GAA protein further comprises a spacer comprising a nucleotide sequence of at least 1 amino acid in length, which is located N-terminal to the GAA polypeptide.

In one embodiment, the spacer at least 50% identical to the sequence GGGTVGDDDDK.

In some embodiments, a spacer or linker can be relatively short, e.g., at least 1, 2, 3, 4 or 5 amino acids, or such as the sequence Gly-Ala-Pro or Gly-Gly-Gly-Gly-Gly-Pro, or can be longer, such as, for example, 5-10 amino acids in length or 10-25 amino acids in length. For example, flexible repeating linkers of 3-4 copies of the sequence (e.g., GGGGS) and a-helical repeating linkers of 2-5 copies of the sequence (e.g., EAAAK) have been described (Arai et al. (2004) Proteins: Structure, Function and Bioinformatics 57:829-838). In some embodiments, a linker comprising GGGTVGDDDDK is also encompassed for use. Linkers incorporating an a-helical portion of a human serum protein can be used to minimize immunogenicity of the linker region. In some embodiments, the spacer is encoded by nucleic acids GGCGCGCCG which encodes the amino acid spacer comprising amino acids GAP or Gly-Ala-Pro.

The site of a fusion junction in the GAA polypeptide to fuse with either the signal peptide should be selected with care to promote proper folding and activity of each polypeptide in the fusion protein and to prevent premature separation of a signal peptide from a GAA polypeptide.

In some embodiments, a spacer has a helical structure. In another specific embodiment, a spacer is at least 50% identical to the sequence GGGTVGDDDDK.

In some embodiments, a signal peptide can be fused, directly or by a spacer, to amino acids of the GAA polypeptide as disclosed in Table 1 herein, permitting expression of the GAA polypeptide or N-terminal truncated GAA polypeptiden, and proper secretion of the GAA polypeptide as described herein in the Examples.

In order to facilitate folding of the signal peptide, GAA amino acid residues adjacent to the fusion junction can be modified. For example, since it is possible that GAA cysteine residues may interfere with proper folding of the signal peptide, the terminal GAA cysteine 952 can be deleted or substituted with serine to accommodate a C-terminal signal peptide. The signal peptide can also be fused immediately preceding the final Cys952. The penultimate cys938 can be changed to proline in conjunction with a mutation of the final Cys952 to serine.

### F. Liver Specific Promoters (LSP)

In some embodiments, to achieve appropriate levels of GAA expression, the rAAV genotype comprises a liver specific promoter (LSP). A LSP enables expression of the operatively linked gene in the liver, and can in some embodiments, be and inducible LSP. In an embodiment, a LSP is located upstream 5′ and is operatively linked to the heterologous nucleic acid sequence encoding the GAA protein.

Exemplary liver-specific promoters useful in the AAV to treat Pompe according to the method disclosed herien are disclosed in International WO2020102645 and WO2021102107, which are incorporated herein in their entirety by reference.

In some embodiments, encompassed herein are any liver-specific promoters disclosed WO2020102645 and WO2021102107, where the LSP has been improved. For example, a liver specific promoter useful in the rAAV vectors as disclosed herein is any LSP disclosed International WO2020102645 and WO2021102107 which has been modified to replace the the sequence of SEQ ID NO: 450 (corresponding to as SEQ ID NO: 126 in WO2021102107 or referred to as CRE0052 or LVR_CRE_0052_G6PC sequence) in any of the LSP sequences in WO2021102107 with a sequence selected from SEQ ID NO: 40 or 41, or a sequence having at least 85%, 90%, 95%, 96%, 97%, 98% or 99% nucleotide sequence identity thereto. Using SP131A1 (or LVR131_A1) promoter as an exemplary promoter, which is disclosed as SEQ ID NO: 94 in WO2021102107, in the current application the promoter has been modified to replace SEQ ID NO 450 (corresponding to SEQ ID NO: 126 in WO2021102107) with SEQ ID NO: 40 or 41, or a sequence having at least 85%, 90%, 95%, 96%, 97%, 98% or 99% nucleotide sequence identity thereto. Any promoter disclosed in WO2021102107 is encompased for use herein, wherein if the promoter comprises SEQ ID NO 450 (corresponding to SEQ ID NO: 126 in WO2021102107), it can be replaced with SEQ ID NO: 40 or 41, or a sequence having at least 85%, 90%, 95%, 96%, 97%, 98% or 99% nucleotide sequence identity thereto.

In some embodiments, the promoter is a LP1 promoter (SEQ ID NO: 432), or a variant having at least sequence at least 85%, 90%, 95%, 96%, 97%, 98% or 99% nucleotide sequence identity thereto.

In some embodiments of the methods to treat Pompe disease disclosed herein, a synthetic liver-specific promoter useful in the AAV vector is any LSP promoter selected from SEQ ID NOS: 86, 88, 91-96, 146-150, 439-441 as disclosed herein, or any LSP selected from SEQ ID NO: 270-341 or 342-430 as disclosed herein, or a synthetic liver-specific promoter thereof which is able to promote liver-specific transgene expression and has an activity in liver cells which is at least 15%, 20%, 30%, 40%, 50%, 60%, 70%, 80%, 90%, 100%, 125%, 150%, 175%, 200%, 250%, 300%, 350% or 400% of the activity of the TTR promoter comprising SEQ ID NO: 431 as International Application WO2021102107, or a synthetic promoter which is disclosed in Table 4 of International Application WO2021102107, which is incorporated herein in its entirity by reference.

In some embodiments, a synthetic liver specific promoter is selected from any of: SEQ ID NOS: 86, 88, 91-96, 146-150, or 270-430 as disclosed herein, or nucleic acid sequence that is at least 80%, or at least 90% or 95% identical thereto or to the source regulatory nucleic acid sequence.

In some embodiments, a liver-specific promoter (LSP) in a AAV expressing a GAA polypeptide as disclosed herein and useful in the methods to treat Pompe disease as disclosed herien comprises a nucleic acid sequence selected from any promoter listed from SEQ ID NOS: 86 (CRM 0412), SEQ ID NO: 91 (SP0412) or SEQ ID NO: 92 (SP0422), SEQ ID NOS: 93 (SP0239), SEQ ID NO: 94 (SP0265), SEQ ID NO: 95 (SP0240) or SEQ ID NO: 96 (SP0246), or SEQ ID NO: 146 (SP0265-UTR), SEQ ID NO: 147 (SP0239-UTR), SEQ ID NO: 148 (SP0240-UTR), SEQ ID NO: 149 (SP0246-UTR) or SEQ ID NO: 150 (SP0131-A1-UTR), SEQ ID NO: 439 (LVR_0243); SEQ ID NO: 440 (LVR_0412) and SEQ ID NO: 441 (A1 Promoter), as disclosed herein, or a functional fragment or variant of any LSP selected from SEQ ID NO: 270-341 or 342-430, or a functional fragment or variant thereof of SEQ ID NOS: 86, 88, 91-96, or 146-150, 439-441 or 270-430.

In some embodiments of the methods to treat Pompe disease disclosed herein, a synthetic liver-specific promoter is selected from any or any LSP promoter selected from SEQ ID NOS: 86 (CRM 0412), SEQ ID NO: 91 (SP0412) or SEQ ID NO: 92 (SP0422), SEQ ID NOS: 93 (SP0239), SEQ ID NO: 94 (SP0265), SEQ ID NO: 95 (SP0240) or SEQ ID NO: 96 (SP0246), or SEQ ID NO: 146 (SP0265-UTR), SEQ ID NO: 147 (SP0239-UTR), SEQ ID NO: 148 (SP0240-UTR), SEQ ID NO: 149 (SP0246-UTR) or SEQ ID NO: 150 (SP0131-A1-UTR), SEQ ID NO: 439 (LVR_0243); SEQ ID NO: 440 (LVR_0412) and SEQ ID NO: 441 (A1 Promoter), or any LSP selected from SEQ ID NO: 270-341 or 342-430 as disclosed herein, where the synthetic liver-specific promoter is able to promote liver-specific transgene expression and has an activity in liver cells which is at least 15%, 20%, 30%, 40%, 50%, 60%, 70%, 80%, 90%, 100%, 125%, 150%, 175%, 200%, 250%, 300%, 350% or 400% of the activity of the TBG promoter of SEQ ID NO: 435.

In some embodiments of the methods to treat Pompe disease disclosed herein, a synthetic liver-specific promoter is selected from any or any LSP promoter selected from any of SEQ ID NO: 97, SEQ ID NO: 98 or SEQ ID NO: 99, or a variant having at least sequence at least 85%, 90%, 95%, 96%, 97%, 98% or 99% nucleotide sequence identity thereto. In some embodiments of the methods to treat Pompe disease disclosed herein, a synthetic liver-specific promoter is selected from any or any LSP promoter selected from SEQ ID NO: 97, SEQ ID NO: 98 or SEQ ID NO: 99, or a variant having at least sequence at least 85%, 90%, 95%, 96%, 97%, 98% or 99% nucleotide sequence identity thereto, where the synthetic liver-specific promoter is able to promote liver-specific transgene expression and has an activity in liver cells which is at least 15%, 20%, 30%, 40%, 50%, 60%, 70%, 80%, 90%, 100%, 125%, 150%, 175%, 200%, 250%, 300%, 350% or 400% of the activity of the TBG promoter of SEQ ID NO: 435.

In some embodiments, to achieve appropriate levels of GAA expression, the rAAV genotype comprises a liver specific promoter (LSP). A LSP enables expression of the operatively linked gene in the liver, and can in some embodiments, be and inducible LSP. In an embodiment, a LSP is located upstream 5′ and is operatively linked to the heterologous nucleic acid sequence encoding the GAA protein. Exemplary liver-specific promoters are disclosed herein, and include for example, the M3 liver specific promoter comprising a sequence of SEQ ID NO: 99, or a functional variant have at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or more of SEQ ID NO: 99.

In one embodiment, the liver promoter is a promoter that has some expression in the liver. In one embodiment, the promoter that has some expression in the liver is the M2 liver promoter comprising a sequence of SEQ ID NO: 98, or a functional variant have at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or more of SEQ ID NO: 98.

In some embodiments, the synthetic liver specific promoter comprises SEQ ID NO: 99, or nucleic acid sequence that is at least 50%, preferably 60%, 70%, 80%, 90% or 95% identical to the source regulatory nucleic acid sequence. In some embodiments, a synthetic liver specific promoter comprises SEQ ID NO: 99, or nucleic acid sequence that is at least 80%, or at least 90% or 95% identical to nucleotides 1-26 of SEQ ID NO: 99.

In some embodiments, a synthetic liver specific promoter that is at least 50%, 60%, 70%, 80%, 90% or 95% identical to SEQ ID NO: 99 comprises a nucleic acid sequence where 2% or 1% or fewer of the nucleotides of SEQ ID NO: 99 are altered. In some embodiments, a synthetic liver-specific promoter useful in the methods and compositions as disclosed herein is the same length, or not substantially altered, or 1, 2, 3, 4, 5, or 6 nucleotides longer or 1, 2, 3, 4, 5, or 6 shorter than the length of SEQ ID NO: 99.

In some embodiments, no nucleotides have been deleted when compared to SEQ ID NO: 99. In some embodiments, no nucleotides are inserted when compared to SEQ ID NO: 99. In some embodiments, all modifications made to SEQ ID NO: 99 are nucleotide substitutions.

In some embodiments, a synthetic liver specific promoter that is at least 50%, 60%, 70%, 80%, 90% or 95% identical to SEQ ID NO: 99 comprises a source regulatory nucleic acid sequence which is active in liver, and the second type of cell or tissue is muscle; or a source regulatory nucleic acid sequence which is active in liver, and the second type of cell or tissue is CNS; or a source regulatory nucleic acid sequence which is active in muscle, and the second type of cell or tissue is liver; or a source regulatory nucleic acid sequence which is active in muscle, and the second type of cell or tissue is CNS.

In some embodiments, a liver-specific promoter which is a functional variant of a given promoter element preferably retains at least 80% of its activity, more preferably at least 90% of its activity, more preferably at least 95% of its activity, and yet more preferably 100% of its activity (compared to the reference promoter comprising the unmodified promoter element). Suitable assays for assessing liver-specific promoter activity are disclosed in Examples 12 and 13 of International Application WO2021102107 which is incorporated herein in its entirity by reference.

In some embodiments, liver specific promoters include, but are not limited to, transthyretin promoter (TTR), LSP promoter (LSP), a synthetic liver specific promoter. For example, in some embodiments of the methods and compositions as disclosed herein, the promoter is a liver specific promoter (LSP), and can be selected from any liver specific promoters including, but not limited to, a transthyretin promoter (TTR), a Liver specific promoter (LSP), for example, as disclosed in U.S. Pat. No. 5,863,541 (TTR promoter), or LSP promoter (PNAS; 96: 3906-3910, 1999. See e.g. p. 3906, Materials and Methods, rAAV construction), a synthetic liver promoter, the references which are incorporated herein in their entireties by reference. Other liver promoters can be used, for example, synthetic liver promoters.

In some embodiments, the TTR promoter is a truncated TTR promoter, e.g., comprising SEQ ID NO: 431, or SEQ ID NO: 12 as disclosed in International WO 2020102645, which is incorporated herein in its entirity by reference, or a variant having at least sequence at least 85%, 90%, 95%, 96%, 97%, 98% or 99% nucleotide sequence identity thereto. In some embodiments, the LSP is a TBG promoter, e.g., comprising SEQ ID NO: 435, or a variant having at least sequence at least 85%, 90%, 95%, 96%, 97%, 98% or 99% nucleotide sequence identity thereto.

Other liver specific promoters include, but are not limited to promoters for the LDL receptor, Factor VIII, Factor IX, phenylalanine hydroxylase (PAH), ornithine transcarbamylase (OTC), and a 1-antitrypsin (hAAT), and HCB promoter. In Other liver specific promoters include the AFP (alpha fetal protein) gene promoter and the albumin gene promoter, as disclosed in EP Patent Publication 0 415 731, the a-1 antitrypsin gene promoter, as disclosed in Rettenger, Proc. Natl. Acad. Sci. 91 (1994) 1460-1464, the fibrinogen gene promoter, the APO-A1 (Apolipoprotein A1) gene promoter, and the promoter genes for liver transference enzymes such as, for example, SGOT, SGPT and g-glutamyle transferase. See also 2001/0051611 and PCT Patent Publications WO 90/07936 and WO 91/02805, which are incorporated herein in their entirety by reference. In some embodiments, the liver specific promoter is a recombinant liver specific promoter, e.g., as disclosed in US20170326256A1, which is incorporated herein in its entirety by reference.

In some embodiments, a liver specific promoter is the hepatitis B X-gene promoter and the hepatitis B core protein promoter. In some embodiments, liver specific promoters can be used with their respective enhancers. The enhancer element can be linked at either the 5′ or the 3′ end of the nucleic acid encoding the GAA polypeptide. The hepatitis B X gene promoter and its enhancer can be obtained from the viral genome as a 332 base pair EcoRV-NcoI DNA fragment employing the methods described in Twu, J Virol. 61 (1987) 3448-3453. The hepatitis B core protein promoter can be obtained from the viral genome as a 584 base pair BamHI-Bglll DNA fragment employing the methods described in Gerlach, Virol 189 (1992) 59-66. It may be necessary to remove the negative regulatory sequence in the BamHI-Bglll fragment prior to inserting it.

It is envisioned that the liver-specific promoter used to express the GAA polypeptide is selected in combination with, or in conjunction with the selection of the signal sequence. In particular, without wishing to be bound by theory, if a strong liver-specific promoter is selected, the signal sequence should be selected that is sufficient to secrete the expressed GAA out of the cell, in order to avoid GAA accumation in the cell and any associated cell toxicity, and/or to avoid the generation of anti-GAA antibodies.

It is encompassed that the LSP is selected in conjunction with the signal sequence, so that the strength of the liver specific promoter (LSP) that is operatively linked to the nucleic acid encoding the GAA polypeptide can be counter-balanced with the ability of the cell to secrete the expressed GAA protein. Thus, if the liver specific promoter is strong, the specific signal sequence must be sufficiently effective to allow for the expressed GAA can be secreted from the cell so that GAA does not accumulate and create cell toxicity and/or induce an immune response. Thus, the cell secretory pathway, and the selected signal sequence must be able to match the level of GAA expressed by the AAV, where the level of GAA expression is dependent on both the AAV transduction efficacy (determined by AAV dose and capsid) and the strength of the liver specific promoter.

### G. UTRs, Regulatory Sequences and Intron Sequences

In some embodiments, the liver-specific promoters as set out above are operably linked to one or more additional regulatory sequences. An additional regulatory sequence can, for example, enhance expression compared to the liver-specific promoter which is not operably linked the additional regulatory sequence. Generally, it is preferred that the additional regulatory sequence does not substantively reduce the specificity of the liver-specific promoter.

For example, the liver-specific promoter can be operably linked to a sequence encoding a UTR (e.g., a 5′ and/or 3′ UTR), an intron, an UTR (e.g., 5′ or 3′)+intron, or such. In some embodiments, the liver-specific promoter is operably linked to sequence encoding a UTR, e.g., a 5′ UTR. A 5′ UTR can contain various elements that can regulate gene expression. The 5′ UTR in a natural gene begins at the transcription start site and ends one nucleotide before the start codon of the coding region. It should be noted that 5′ UTRs as referred to herein may be an entire naturally occurring 5′ UTR or it may be a portion of a naturally occurring 5′ UTR. The 5′UTR can also be partially or entirely synthetic. In eukaryotes, 5′ UTRs have a median length of approximately 150 nt, but in some cases they can be considerably longer. Regulatory sequences that can be found in 5′ UTRs are disclosed in International Application WO2021102107 which is incorporated herein in its entirity by reference.

In some embodiments, a 5-UTR sequence is located 3′ of a liver specific promoter as disclosed herein, and 5′ of the heterologous nucleic acid sequence (e.g., encoding a signal peptide and GAA polypeptide).

In one embodiment, an exemplary 5-UTR sequence comprises, for example, a 24 bp sequence of SEQ ID NO: 41, or a functional variant have at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or more of SEQ ID NO: 41.

In one embodiment, an exemplary 5-UTR sequence comprising SEQ ID NO: 41 is the sequence of SEQ ID NO: 40, or a functional variant have at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or more of SEQ ID NO: 40.

In some embodiments, the 5-UTR sequence comprises SEQ ID NO: 41 or SEQ ID NO: 40, or nucleic acid sequence that is at least 50%, preferably 60%, 70%, 80%, 90% or 95% identical to the source regulatory nucleic acid sequence. In some embodiments, a 5-UTR sequence comprises SEQ ID NO: 41 or SEQ ID NO: 40 or nucleic acid sequence that is at least 80%, or at least 90% or 95% identical to nucleotides of SEQ ID NO: 41 or SEQ ID NO: 40.

In some embodiments, a 5-UTR that is at least 50%, 60%, 70%, 80%, 90% or 95% identical to SEQ ID NO: 41 or SEQ ID NO: 40 comprises a nucleic acid sequence where 2% or 1% or fewer of the nucleotides of SEQ ID NO: 41 or SEQ ID NO: 40 are altered. In some embodiments, a 5-UTR sequence useful in the methods and compositions as disclosed herein is the same length, or not substantially altered, or 1, 2, 3, 4, 5, or 6 nucleotides longer or 1, 2, 3, 4, 5, or 6 shorter than the length of SEQ ID NO: 41 or SEQ ID NO: 40.

Introns within 5′ UTRs have been linked to regulation of gene expression and mRNA export. In some embodiments, a liver-specific promoter as set out above is operably linked to a sequence encoding a 5′ UTR derived from the CMV major immediate gene (CMV-IE gene). For example, the 5′ UTR from the CMV-IE gene suitably comprises the CMV-IE gene exon 1 and the CMV-IE gene exon 1, or portions thereof. In some cases, the promoter element may be modified in view of the linkage to the 5′UTR, for example sequences downstream of the transcription start site (TSS) in the promoter element can be removed (e.g. replaced with the 5′ UTR).

The CMV-IE 5′UTR is described in Simari, et al, Molecular Medicine 4: 700-706, 1998 “Requirements for Enhanced Transgene Expression by Untranslated Sequences from the Human Cytomegalovirus Immediate-Early Gene”, which is incorporated herein by reference. Variants of the CMV-IE 5′ UTR sequences discussed in Simari, et al. are also set out in WO2002/031137, incorporated by reference, and the regulatory sequences disclosed therein can also be used. Other UTRs that can be used in combination with a promoter are known in the art, e.g. in Leppek, K., Das, R. & Bama, M. “Functional 5′ UTR mRNA structures in eukaryotic translation regulation and how to find them”. Nat Rev Mol Cell Biol 19, 158-174 (2018), incorporated by reference.

In some embodiments the sequence encoding the 5′ UTR comprises SEQ ID NO: 145 as disclosed herein, or a functional variant thereof. In some embodiments, functional variants may have a sequence that is at least 60%, 65%, 70%, 75%, 80%, 85%, 90%, 95%, 96%, 97%, 98%, or 99% identical thereto. SEQ ID NO: 145 as disclosed herein encodes a CMV-IE 5′ UTR.

In some embodiments the sequence encoding the 5′ UTR comprises SEQ ID NO: 446 as disclosed herein, or a functional variant thereof. In some embodiments, functional variants may have a sequence that is at least 60%, 65%, 70%, 75%, 80%, 85%, 90%, 95%, 96%, 97%, 98%, or 99% identical thereto. SEQ ID NO: 446 as disclosed herein, which is a modified CMV-IE intron sequence.

In some embodiments the 5′ UTR comprises a nucleic acid motif that functions as the protein translation initiation site, e.g. sequences that define a Kozak sequence in the mRNA produced. For example, in some embodiments, the sequence encoding the 5′ UTR comprises the sequence motif GCCACC at or near its 3′ end. Other Kozak sequences or other protein translation initiation sites can be used, as is known in the art (e.g. Marilyn Kozak, “Point Mutations Define a Sequence Flanking the AUG Initiator Codon That Modulates Translation by Eukaryotic Ribosomes” Cell, Vol. 44, 283-292, Jan. 31, 1986; Marilyn Kozak “At Least Six Nucleotides Preceding the AUG Initiator Codon Enhance Translation in Mammalian Cells” J. Mol. Rid. (1987) 196, 947-950; Marilyn Kozak “An analysis of 5′-noncoding sequences from 699 vertebrate messenger RNAs” Nucleic Acids Research. Vol. 15 (20) 1987, all of which are incorporated herein by reference). The protein translation initiation site (e.g. Kozak sequence) is preferably positioned immediately adjacent to the start codon.

In some embodiments, a sequence encoding a 5′ UTR comprises SEQ ID NO: 438 as disclosed herein, or a functional variant thereof. In some embodiments, functional variants may have a sequence that is at least 60%, 65%, 70%, 75%, 80%, 85%, 90%, 95%, 96%, 97%, 98%, or 99% identical thereto. This 5′ UTR comprises six nucleotides of GCCACC, which define a Kozak sequence at the 3′ end of the CMV-IE 5′ UTR.

In some embodiments, the rAAV expressing GAA for use in the methods to treat Pompe as disclosed herien comprises an intron sequence located 3′ of the promoter sequence and 5′ of the heterologous nucleic acid (i.e., 5′ of the nucleic acid encoding the signal peptide and GAA polypeptide). Intron sequences serve to increase one or more of: mRNA stability, mRNA transport out of nucleus and/or expression and/or regulation of the expressed GAA polypeptide. In alternative embodiments, a rAAV genotype does not comprise an intron sequence.

In one embodiment, a UTR sequence described herein can be used as a 3′UTR.

A synthetic liver-specific promoter according to the present invention can be operably linked to a sequence encoding a UTR (e.g. a 5′ and/or 3′ UTR), and/or an intron, or suchlike. In some embodiments, a synthetic liver specific promoter as set herein, is operably linked to a sequence encoding a 5′ UTR and an intron. In some embodiments, the 5′ UTR and intron is derived from the CMV major immediate gene (CMV-IE gene). The CMV-IE 5′UTR and intron is described in Simari, et al., Molecular Medicine 4: 700-706, 1998 “Requirements for Enhanced Transgene Expression by Untranslated Sequences from the Human Cytomegalovirus Immediate-Early Gene”, which is incorporated herein by reference. Variants of the CMV-IE 5′ UTR and intron sequences discussed in Simari, et al. are also set out in WO2002/031137, incorporated by reference, and the regulatory sequences disclosed therein can also be used. In some embodiments the 5′ UTR or the 5′ UTR and intron suitably comprises a nucleic acid motif that functions as the protein translation initiation site, e.g. sequences that define a Kozak sequence in the mRNA produced. For example, in some embodiments, the sequence encoding the 5′ UTR comprises the sequence motif GCCACC at or near its 3′ end. Other Kozak sequences or other protein translation initiation sites can be used, as is known in the art (e.g. Marilyn Kozak, “Point Mutations Define a Sequence Flanking the AUG Initiator Codon That Modulates Translation by Eukaryotic Ribosomes” Cell, Vol. 44, 283-292, Jan. 31, 1986; Marilyn Kozak “At Least Six Nucleotides Preceding the AUG Initiator Codon Enhance Translation in Mammalian Cells” J. Mol. Rid. (1987) 196, 947-950; Marilyn Kozak “An analysis of 5”-noncoding sequences from 699 vertebrate messenger RNAs” Nucleic Acids Research. Vol. 15 (20) 1987, all of which are incorporated herein by reference). The protein translation initiation site (e.g. Kozak sequence) is preferably positioned immediately adjacent to the start codon.

In some embodiments, any one of the promoters described herein, or variants thereof, is linked to a sequence encoding a 5′ UTR and/or a 5′UTR and an intron to provide a composite promoter. Herein, such composite promoter may be referred to simply as “composite promoters”, or in some cases simply “promoters” for brevity.

In some embodiments, the intron sequence is a MVM intron sequence, for example, but not limited to intron sequence of SEQ ID NO: 442, or nucleic acid sequence having at least 80%, 85%, 90%, 95%, 96%, 97%, 98% or 99% nucleotide sequence identity thereto.

In some embodiments, the intron sequence is a HBB2 intron sequence, for example, but not limited to and intron sequence of SEQ ID NO: 443 or SEQ ID NO: 444 or nucleic acid sequence having at least 80%, 85%, 90%, 95%, 96%, 97%, 98% or 99% nucleotide sequence identity thereto.

In some embodiments of the methods and compositions disclosed herein, a recombinant AAV vector comprises a heterologous nucleic acid sequence that further comprises an intron sequence located 5′ of the sequence encoding the secretory signal peptide, and 3′ of the promoter. In some embodiments, the intron sequence comprises a MVM sequence or a HBB2 sequence, wherein the MVM sequence comprises the nucleic acid sequence of SEQ ID NO: 442, or a nucleic acid sequence at least about 75%, or 80%, or 85%, or 90%, or 95%, or 98%, or 99% sequence identity to SEQ ID NO: 442, and the HBB2 sequence comprises the nucleic acid sequence of SEQ ID NO: 443 or SEQ ID NO: 444, or a nucleic acid sequence at least about 75%, or 80%, or 85%, or 90%, or 95%, or 98%, or 99% sequence identity to SEQ ID NO: 443 or SEQ ID NO: 444.

In some embodiments, the intron sequence is a ubiquitin C (UBC) intron sequence, e.g., intron 1 from the UBC gene, or a portion thereof, e.g., as disclosed in Bianchi et al, 2009, Gene, 448 (1); 88-101, where the intron 1 sequence of the UBC gene is 812 bp and starts at chromosomal location 124,914,586, and ends at 124,913,775. In some embodiments, the intron sequence is a UBC intron, for example, but not limited to intron sequence of SEQ ID NO: 445, or nucleic acid sequence having at least 80%, 85%, 90%, 95%, 96%, 97%, 98% or 99% nucleotide sequence identity to SEQ ID NO: 445.

In some embodiments, the rAAV genotype comprises an intron sequence selected in the group consisting of a human beta globin b2 (or HBB2) intron, a FIX intron, a chicken beta-globin intron, a CMVIE intron, a UBC intron, a HBB intron sequence, a MVM sequence and a SV40 intron. In some embodiment, the intron is intron 1 from human RNA pol II. In some embodiments, the intron is optionally a modified intron such as a modified HBB2 intron (see, e.g., SEQ ID NO: 17 in of WO2018046774A1): a modified FIX intron (see., e.g., SEQ ID NO: 19 in WO2018046774A1), or a modified chicken beta-globin intron (e.g., see SEQ ID NO: 21 in WO2018046774A1), or modified HBB2 or FIX introns disclosed in WO2015/162302, which are incorporated herein in their entirety by reference.

### H. Poly-A Sequences and Terminator Sequences

In some embodiments, an rAAV vector genome includes at least one poly-A tail that is located 3′ and downstream from the heterologous nucleic acid gene encoding the GAA polypeptide. Any polyA sequence can be used, including but not limited to hGH poly A, BGH poly A, SV40 poly A, synpA polyA and the like. In some embodiments, the polyA is a synthetic polyA sequence. In some embodiments, the rAAV vector genome comprises two poly-A tails, e.g., a hGH poly A sequence and another polyA sequence, where a spacer nucleic acid sequence is located between the two poly A sequences.

In some embodiments, the polyA signal is 3′ of the heterologous nucleic acid sequence encoding the GAA polypeptide. In some embodiments, the rAAV genome comprises 3′ of the nucleic acid encoding the GAA polypeptide, a first polyA sequence and a reverse RNA polymerase II terminator sequence (rev RNA PolII terminator sequence), and the 3′ ITR. Non limiting examples of first polyA is hGH poly A, BGH poly A, SV40 poly A or, any functional fragment thereof in 5′ to 3′ orientation. Non limiting examples of reverse RNA polymerase II terminator sequence is hGH poly A, BGH poly A, SV40 poly A or, any functional fragment thereof in 3′ to 5′ orientation.

In some embodiments, the rAAV genome comprises 3′ of the nucleic acid encoding the GAA polypeptide, a first polyA sequence, a spacer nucleic acid sequence (e.g., of between 100-400 bp, or about 100-250 bp, or about 250-400 bp), a second poly A sequence, a spacer nucleic acid sequence, and the 3′ ITR.

In some embodiments, the first and/or second poly A sequence is a hGH poly A sequence, and in some embodiments, the first and second poly A sequences are a synthetic poly A sequence. In some embodiments, the first poly A sequence is a hGH poly A sequence and the second poly A sequence is a synthetic sequence, or vice versa—that is, in alternative embodiments, the first poly A sequence is a synthetic poly A sequence and the second poly A sequence is a hGH polyA sequence.

In some embodiments, the poly A sequence is selected from any of: SEQ ID NO: 42, SEQ ID NO: 43 or SEQ ID NO: 44, where SEQ ID NO: 44 comprises the signal AATAAA, or a poly A nucleic acid sequence having at least 80%, 85%, 90%, 95%, 96%, 97%, 98% or 99% nucleotide sequence identity to any of SEQ ID NOS: 42, 43 or 44.

In some embodiments, the poly A sequence is selected from any of: SEQ ID NO: 46 or SEQ ID NO: 47, or a poly A nucleic acid sequence having at least 80%, 85%, 90%, 95%, 96%, 97%, 98% or 99% nucleotide sequence identity to any of SEQ ID NOS: 46 or 47.

In some embodiments, the poly A sequence is, for example, SEQ ID NO: 15 as disclosed in International WO2021102107 (hGH poly A sequence), or a poly A nucleic acid sequence having at least 80%, 85%, 90%, 95%, 96%, 97%, 98% or 99% nucleotide sequence identity to SEQ ID NO: 15 as disclosed in International Application WO2021102107. In some embodiments, the hGHpoly sequence encompassed for use is described in Anderson et al. J. Biol. Chem 264(14); 8222-8229, 1989 (See, e.g., p. 8223, 2nd column, first paragraph) which is incorporated herein in its entirety by reference.

In one embodiment, the recombinant AAV disclosed herein comprises in its genome a transcriptional terminator signal sequence or a transcriptional pause signal sequence in the reverse orientation between polyA and 3′ITR. In one embodiment, the recombinant AAV disclosed herein comprises in its genome a transcriptional terminator signal sequence or a transcriptional pause signal sequence that is in the 3′-5′ orientation between polyA and 3′ITR. Any transcription termination signal can be used including, e.g., inverted natural polyA sequences from any species or synthetic polyA signals or fragments thereof, or other nucleic acid structure terminators known in the art. Exemplary polyA signals and/or transcription terminators include, but are not limited to the polyA signals of BGH, SV40, HGH, Betaglobin, RNA polymerase II transcriptional pause signal from alpha 2 globin gene, transcription termination signal for pol III, or fragments thereof, and in any combination thereof.

In some embodiments, a transcriptional terminator signal sequence is a reverse RNA polymerase II terminator sequence which is, in a 5′ to 3′ orientation SEQ ID NO: 45, or a rev RNA PolII terminator sequence having at least 80%, 85%, 90%, 95%, 96%, 97%, 98% or 99% nucleotide sequence identity to any of SEQ ID NOS: 45, where SEQ ID NO: 45 orientated in a 5′ to 3′ direction is located between the 3′ of the poly A sequence and 5′ of the right ITR sequence (or 3′ ITR).

A transcription terminator signal or reverse RNA Polymerase II terminator sequence as described here is also interchangeably be called a “reverse poly A,” which refers to a polyA signal sequence placed in a 3′-5′ orientation downstream of the nucleic acid encoding GAA and upstream of 3′ITR. Any natural or synthetic poly A in 3′-5′ orientation can be used as reverse poly A. In some embodiments, the reverse poly A is the poly A (pA) as described in International publication no. WO2019143950 and US application publication no. US20200340013, which are incorporated herein by reference in its entirety.

For the sake of clarity, “reverse poly A,” “the double stranded RNA termination element,” and “reverse RNA Polymerase II terminator sequence” are used interchangeably herein. In 3′ to 5′ orientation, the reverse poly A or termination element does not allow transcription from 3′ITR, and hence double stranded RNA is not transcribed from 3′ITR. The reverse poly A or double stranded RNA termination element can be heterologous, e.g., from a different gene, for example, other than the gene of interest, or homologous to, e.g., the same gene as the gene of interest. In various embodiments, the poly A signal comprises the double stranded RNA transcription element or reverse poly A. For example, the poly A signal of several aspects of the invention described herein comprises a full length poly A signal in 5′ to 3′ orientation and another poly A signal in 3′ to 5′ orientation. In some embodiments, the 5′ end of double stranded RNA termination element or reverse poly A sequence, and the 3′ end of poly A signal are immediately next to each other, or at least 1 nucleotide apart, or at least 2 nucleotides apart, or at least 3 nucleotides apart, or at least 4 nucleotides apart, or at least 5 nucleotides apart, or or at least 6 nucleotides apart, or at least 7 nucleotides apart, or at least 8 nucleotides apart, or at least 9 nucleotides apart, or at least 10 nucleotides apart, or more apart. In some embodiments, the poly A signal does not comprise double stranded RNA transcription element or reverse poly A. In some embodiments, the poly A signal comprises AATAAA (SEQ ID NO: 467) or AAUAAA (SEQ ID NO: 468). In some embodiments, the poly A signal comprises at least 1, at least 2, at least 3, at least 4, at least 5, at least 6, at least 7, at least 8, at least 9, at least 10 repeats or more of AATAAA (SEQ ID NO: 467) or AAUAAA (SEQ ID NO: 468). In some embodiments, the poly A signal comprises transcription termination signal for Pol III as described in “Delineation of the Exact Transcription Termination Signal for Type 3 Polymerase III. Mol Ther Nucleic Acids. 2018 Mar. 2; 10:36-44, which is incorporated herein by reference in its entirety. In some embodiments, the one or more transcription termination signals for Pol III is in 3′ to 5′ orientation. In some embodiments, the poly A signal comprises TTTT. In some embodiments, poly A signal comprises AAAAAAA (SEQ ID NO: 469). The poly A sequences as described in “Definition of an efficient synthetic poly(A) site” Genes Dev. 1989 July; 3(7):1019-25. doi: 10.1101/gad.3.7.1019., which is incorporated by reference in its entirety. All the above poly A sequences and or terminator sequences described herein can be used as inverted sequence e.g., in 3′ to 5′ orientation.

In some aspects of the invention descried herein, the poly A sequence comprises poly A sequence and a terminator sequence, e.g., the poly A sequence comprises hGH Poly A sequence and a Pol III terminator sequence. In various aspects of the invention, the poly A sequence and Pol III terminator sequences are interchangeably referred to as “poly A.” In several aspects of the invention described herein, the poly A sequence further comprises a Reverse RNA Polymerase II terminator sequence, or RNA Polymerase II transcriptional pause signal sequence, or reverse poly A. Without any limitation, an example of Reverse RNA Polymerase II terminator sequence, or RNA Polymerase II transcriptional pause signal sequence, or reverse Poly A, is the 3′ sequence of the human hemoglobin alpha gene.

In some embodiments, a poly-A tail can be engineered to stabilize the RNA transcript that is transcribed from an rAAV vector genome, including a transcript for a heterologous gene, which in one embodiment is a GAA, and in alternative embodiments, the poly-A tail can be engineered to include elements that are destabilizing.

In one embodiment, the polyA is a bi-directional polyA sequence. Bi-directional polyA sequences are commonly isolated from virual DNA, for example, the SV40 polyA is a bi-directional polyA.

In some embodiments of the methods to treat Pompe disease as disclosed herein, a recombinant AAV vector comprises at least one polyA sequence located 3′ of the nucleic acid encoding the GAA gene and 5′ of the 3′ ITR sequence. In some embodiments, the poly A is a full length poly A (fl-polyA) sequence. In some embodiments, the polyA is a truncated polyA sequence as disclosed in International WO2021102107, which is incorporated herein in its entirity.

In an embodiment, a poly-A tail can be engineered to become a destabilizing element by altering the length of the poly-A tail. In an embodiment, the poly-A tail can be lengthened or shortened.

In some embodiments, there is a 3′ untranslated regions (3′UTRs) located between the heterologous gene encoding the GAA polypeptide and the poly-A tail. In some embodiments, there is a 3′ UTR located 3′ of the nucleic acid sequence encoding the GAA polypeptide. In some embodiments, a 3′ untranslated region (3′UTR) comprises GAA 3′ UTR (SEQ ID NO: 50) or a 3′ UTR (SEQ ID NO: 49) as disclosed herein.

In another embodiment, a promoter region, or 3′ UTR or polyA region can comprise a destabilizing element, is a target sequence for a microRNA (miRNA) that has the ability to silence (repress translation and promote degradation) the RNA transcripts when the miRNA binds to a miRNA target sequence. Accordingly, in some embodiments, addition or deletion of seed regions within the 3-UTR or a poly-A tail can increase or decrease expression of a protein, such as the GAA polypeptide. In some embodiments, the miRNA target region is a synthetic miRNA target region which is targeted by an artificial miRNA (amiRNA) according to methods known in the art.

In another embodiment, seed regions can also be engineered into the 3′ untranslated regions (3′UTRs) located between the heterologous gene and the poly-A tail. In a further embodiment, the destabilizing agent can be an siRNA. The coding region of the siRNA can be included in an rAAV vector genome and is generally located downstream, 3′ of the poly-A tail.

In all aspects of the methods for treating Pompe disease as disclosed herein, the rAAV genome may also comprise a Stuffer DNA nucleic sequence. An exemplary stuffer DNA sequence is SEQ ID NO: 71 as disclosed in International Application WO2021102107, or a nucleic acid sequence having at least 80%, 85%, 90%, 95%, 96%, 97%, 98% or 99% nucleotide sequence identity thereto.

In some embodiments, the stuffer sequence is located 3′ of the poly A tail, for example, and is located 5‘ of the’3 ITR sequence. In some embodiments, the stuffer DNA sequence comprises a synthetic polyadenylation signal in the reverse orientation.

In some embodiments, a stuffer nucleic acid sequence (also referred to as a “spacer” nucleic acid fragment) can be located between the poly A sequence and the 3′ ITR (i.e., a stuffer nucleic acid sequence is located 3′ of the polyA sequence and 5′ of the 3′ ITR). Such a stuffer nucleic acid sequence can be about 30 bp, 50 pb, 75 bp, 100 bp, 150 bp, 200 bp, 250 bp, 300 bp or longer than 300 bp. In some embodiments of the methods and compositions as disclosed herein, a stuffer nucleic acid fragment is between 20-50 bp, 50-100 bp, 100-200 bp, 200-300 bp, 300-500 bp, or any integer between 20-500 bp. Exemplary stuffer (or spacer) nucleic acid sequence can be selected from any of: SEQ ID NO: 16, SEQ ID NO: 71 or SEQ ID NO: 78 as disclosed in International Application WO2021102107, or a nucleic acid sequence at least about 70, 71, 72, 73, 74, 75, 76, 77, 78, 79, 80, 81, 82, 83, 84, 85, 86, 87, 88, 89, 90, 91, 92, 93, 94, 95, 96, 97, 98, 99%, identical to SEQ ID NO: 16 or SEQ ID NO: 71 or SEQ ID NO: 78 as disclosed in International Application WO2021102107.

### E. CS Sequence

In some embodiments of the methods and compositions disclosed herein, a recombinant AAV vector comprises a heterologous nucleic acid sequence that can further comprises at collagen stability (CS) sequence located 3′ of the nucleic acid encoding the GAA polypeptide and 5′ of the 3′ ITR sequence. In some embodiments, the rAAV genome disclosed herein comprises a heterologous nucleic acid sequence that can optionally comprise a Collagen stability sequence (CS or CSS), which is positioned 3′ of the nucleic acid encoding the GAA polypeptide and 5′ of the nucleic acid encoding a polyA signal. In some embodiments, the CS sequence can be replaced by a 3′ UTR sequence as disclosed herein.

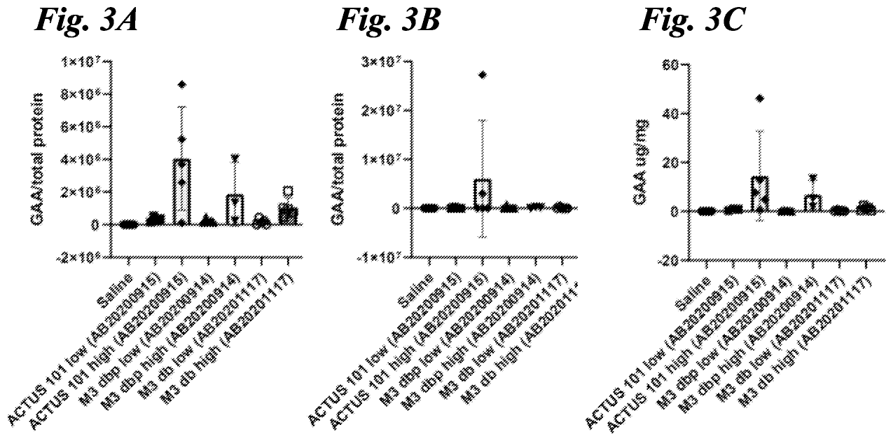

Exemplary collagen stability sequences include CCCAGCCCACTTTTCCCCAA or a sequence at least 85%, 90%, 95%, 96%, 97%, 98% or 99% sequence identity thereto. An exemplary collagen stability sequence can have an amino acid sequence of PSPLFP or an amino acid sequence having at least 85%, 90%, 95%, 96%, 97%, 98% or 99% sequence identity thereto. CS sequences are disclosed in Holick and Liebhaber, Proc. Nat. Acad. Sci. 94: 2410-2414, 1997 (See, e.g. FIG. 3, p. 5205), which is incorporated herein its entirety by reference.

### I. AAV ITRs

The rAAV vector or genome as disclosed herein for use in the methods to treat Pompe disease can comprise AAV ITRs that have desirable characteristics and can be designed to modulate the activities of, and cellular responses to vectors that incorporate the ITRs. In another embodiment, the AAV ITRs are synthetic AAV ITRs that has desirable characteristics and can be designed to manipulate the activities of and cellular responses to vectors comprising one or two synthetic ITRs, including, as set forth in U.S. Pat. No. 9,447,433, which is incorporated herein by reference.

In another embodiment, an ITR exhibits modified transcription activity relative to a naturally occurring ITR, e.g., ITR2 from AAV2. It is known that the ITR2 sequence inherently has promoter activity. It also inherently has termination activity, similar to a poly(A) sequence. The minimal functional ITR of the present invention exhibits transcription activity as shown in the examples, although at a diminished level relative to ITR2. Thus, in some embodiments, the ITR is functional for transcription. In other embodiments, the ITR is defective for transcription. In certain embodiments, the ITR can act as a transcription insulator, e.g., preventing transcription of a transgenic cassette present in the vector when the vector is integrated into a host chromosome.

One aspect of the invention relates to an rAAV vector genome comprising at least one synthetic AAV ITR, wherein the nucleotide sequence of one or more transcription factor binding sites in the ITR is deleted and/or substituted, relative to the sequence of a naturally occurring AAV ITR such as ITR2. In some embodiments, it is the minimal functional ITR in which one or more transcription factor binding sites are deleted and/or substituted. In some embodiments at least 1 transcription factor binding site is deleted and/or substituted, e.g., at least 5 or more or 10 or more transcription factor binding sites, e.g., at least 1, 2, 3, 4, 5, 6, 7, 8, 9, 10, 11, 12, 13, 14, 15, 16, 17, 18, 19, 20, or 21 transcription factor binding sites.

Another embodiment, a rAAV vector, including an rAAV vector genome as described herein comprises a polynucleotide comprising at least one synthetic AAV ITR, wherein one or more CpG islands (a cytosine base followed immediately by a guanine base (a CpG) in which the cytosines in such arrangement tend to be methylated) that typically occur at, or near the transcription start site in an ITR are deleted and/or substituted. In an embodiment, deletion or reduction in the number of CpG islands can reduce the immunogenicity of the rAAV vector. This results from a reduction or complete inhibition in TLR-9 binding to the rAAV vector DNA sequence, which occurs at CpG islands. It is also well known that methylation of CpG motifs results in transcriptional silencing. Removal of CpG motifs in the ITR is expected to result in decreased TLR-9 recognition and/or decreased methylation and therefore decreased transgene silencing. In some embodiments, it is the minimal functional ITR in which one or more CpG islands are deleted and/or substituted. In an embodiment, AAV ITR2 is known to contain 16 CpG islands of which one or more, or all 16 can be deleted.

In some embodiments, at least 1 CpG motif is deleted and/or substituted, e.g., at least 4 or more or 8 or more CpG motifs, e.g., at least 1, 2, 3, 4, 5, 6, 7, 8, 9, 10, 11, 12, 13, 14, 15, or 16 CpG motifs.

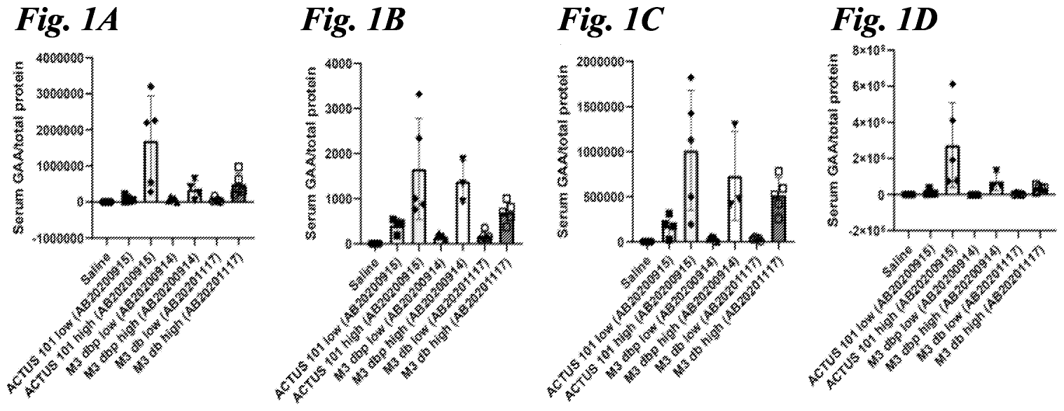

In another embodiment, the synthetic ITR comprises, consists essentially of, or consists of one of the nucleotide sequences listed in Table 4. In other embodiments, the synthetic ITR comprises, consist essentially of, or consist of a nucleotide sequence that is at least 80% identical, e.g., at least 85%, 90%, 95%, 96%, 97%, 98%, or 99% identical to any one of the nucleotide sequences listed in Table 4. In some embodiments, the ITR is a sequence is disclosed in FIG. 1 of Samulski et al, 1983, Cell, 33; 135-143 (referred to “Samulski et al, 1983” as which is incorporated herein in its entirety by reference), which discloses modified ITR sequences in FIG. 1. In some embodiments, the ITR sequence comprises, or consists of a nucleotide sequence that is at least 80% identical, e.g., at least 85%, 90%, 95%, 96%, 97%, 98%, or 99% identical to one of the ITR sequences in FIG. 1 as disclosed in Samulski et al, 1993. In some embodiments, the ITR comprises, or consists of a nucleotide sequence that is at least 80% identical, e.g., at least 85%, 90%, 95%, 96%, 97%, 98%, or 99% or 99.5% identical to the ITR sequence of pSM 609 right disclosed in the middle panel of FIG. 1 (that lacks the 9 bp) disclosed in Samulski et al, 1983. In some embodiments, the ITR comprises a nucleotide sequence that is at least 80% identical, e.g., at least 85%, 90%, 95%, 96%, 97%, 98%, or 99% or 99.5% identical to the ITR sequence of any of SEQ ID NOs: 79-84 and 450-451.

In some embodiments, the ITR sequence, e.g., Right ITR (or 3′ ITR) is SEQ ID NO: 80 or SEQ ID NO: 82 or a nucleotide sequence that is at least 80% identical, e.g., at least 85%, 90%, 95%, 96%, 97%, 98%, or 99% or 99.5% identical to SEQ ID NO: 80 or SEQ ID NO: 82. In some embodiments, the ITR sequence, e.g., left ITR (or 5′ ITR) is SEQ ID NO: 79 or SEQ ID NO: 81 or a nucleotide sequence that is at least 80% identical, e.g., at least 85%, 90%, 95%, 96%, 97%, 98%, or 99% or 99.5% identical to SEQ ID NO: 79 or SEQ ID NO: 81.

TABLE 4
Exemplary synthetic ITR sequences

| | |
|---|---|
| L-ITR | CTGCGCGCTCGCTCGCTCACTGAGGCCGCCCGGGCAAAGCCCGGGCGTC |
| | GGGCGACCTTTGGTCGCCCGGCCTCAGTGAGCGAGCGAGCGCGCAGAGA |
| | GGGAGTGGCCAACTCCATCACTAGGGGTTCCT (SEQ ID NO: 79) |
| R-ITR | AGGAACCCCTAGTGATGGAGTTGGCCACTCCCTCTCTGCGCGCTCGCTCG |
| | CTCACTGAGGCCGGGCGACCAAAGGTCGCCCGACGCCCGGGCTTTGCCC |
| | GGGCGGCCTCAGTGAGCGAGCGAGCGCGCAG (SEQ ID NO: 80) |
| L-ITR | CTGCGCGCTCGCTCGCTCACTGAGGCCGCCCGGGCAAAGCCCGGGCGTC |
| | GGGCGACCTTTGGTCGCCCGGCCTCAGTGAGCGAGCGAGCGCGCAGAGA |
| | GGGAGTGGCCAACTCCATCACTAGGG (SEQ ID NO: 81) |
| R-ITR | AGGAACCCCTAGTGATGGAGTTGGCCACTCCCTCTCTGCGCGCTCGCTCG |
| | CTCACTGAGGCCGGGCGACCAAAGGTCGCCCGACGCCCGGGCTTTGCCC |
| | GGGCGGCCTCAGTGAGCGAGCGAGCGCGCAG (SEQ ID NO: 82) |
| MH-257 | AGGAACCCCTAGTGATGGAGTTGGCCACTCCCTCTCTGCGCGCTCGCTCG |
| | CTCACTGAGGCAATTTGATAAAAATCGTCAAATTATAAACAGGCTTTGCC |
| | TGTTTAGCCTCAGTGAGCGAGCGAGCGCGCAGAGAGGGAGTGGCCAACT |
| | CCATCACTAGGGGTTCCT (SEQ ID NO: 83) |
| MH-258 | AGGAACCCCTAGTGATGGAGTTGGCCACTCCCTCTCTGCGCGCTCGCTCG |
| | CTCACTGAGGGATAAAAATCCAGGCTTTGCCTGCCTCAGTGAGCGAGCG |
| | AGCGCGCAGAGAGGGAGTGGCCAACTCCATCACTAGGGGTTCCT (SEQ |
| | ID NO: 84) |
| MH Delta | AGGAACCCCTAGTGATGGAGTTGGCCACTCCCTCTCTGCGCGCTCGCTCG |
| 258 | CTCACTGAGGGATAAAAATCCAGGCTTTGCCTGCCTCAGTGAGCGAGCG |
| | AGCGCGCAGAGAGGGAGTGGCCAACTCCATCACTAGGGGTTCCT (SEQ |
| | ID NO: 85) |
| MH Telomere- | AGGAACCCCTAGTGATGGAGTTGGCCACTCCCTCTCTGGGATTGGGATTG |
| 1 ITR | CGCGCTCGCTCGCGGGATTGGGATTGGGATTGGGATTGGGATTGGGATTG |
| | ATAAAAATCAATCCCAATCCCAATCCCAATCCCAATCCCAATCCCGCGAG |
| | CGAGCGCGCAATCCCAATCCCAGAGAGGGAGTGGCCAACTCCATCACTA |
| | GGGGTTCCT (SEQ ID NO: 86) |
| MH Telomere- | AGGAACCCCTAGTGATGGAGTTGGCCACTCCCTCTCTGCGCGCTCGCTCG |
| 2 ITR | CTCGGGATTGGGATTGGGATTGGGATTGGGATTGGGATTGATAAAAATC |
| | AATCCCAATCCCAATCCCAATCCCAATCCCAATCCCGCGAGCGAGCGCGC |
| | AGGAGAGGGAGTGGCCAACTCCATCACTAGGGGTTCCTAAGCTTATTAT |
| | A (SEQ ID NO: 87) |
| MH PolII 258 | AGGAACCCCTAGTGATGGAGTTGGCCACTCCCTCTCTGCGCGCTCGCTCG |
| ITR | CTCACTGAGGGCGCCTATAAAGATAAAAATCCAGGCTTTGCCTGCCTCAG |
| | TTAGCGAGCGAGCGCGCAGAGAGGGAGTGGCCAACTCCATCACTAGGGG |
| | TTCCT (SEQ ID NO: 88) |
| MH 258 | CTAGTGATGGAGTTGGCCACTCCCTCTCTGCGCGCTCGCTCGCTCACTGA |
| Delta D | GGGATAAAAATCCAGGCTTTGCCTGCCTCAGTGAGCGAGCGAGCGCGCA |
| conservative | GAGAGGGAGTGGCCAACTCCATCACTAG (SEQ ID NO: 89) |
| 5′ ITR | TTGGCCACTCCCTCTCTGCGCGCTCGCTCGCTCACTGAGGCCGGGCGACC |
| | AAAGGTCGCCCGACGCCCGGGCTTTGCCCGGGCGGCCTCAGTGAGCGAG |
| | CGAGCGCGCAGAGAGGGAGTGGCCAACTCCATCACTAGGGGTTCCT (SEQ |
| | ID NO: 104) |
| 3′ ITR | AGGAACCCCTAGTGATGGAGTTGGCCACTCCCTCTCTGCGCGCTCGCTCG |
| | CTCACTGAGGCCGGGCGACCAAAGGTCGCCCGACGCCCGGGCTTTGCCC |
| | GGGCGGCCTCAGTGAGCGAGCGAGCGCGCAGAGAGGGAGTGGCCAA |
| | (SEQ ID NO: 105) |
| ITR (145 bp) | AGGAACCCCTAGTGATGGAGTTGGCCACTCCCTCTCTGCGCGCTCGCTCG |
| | CTCACTGAGGCCGGGCGACCAAAGGTCGCCCGACGCCCGGGCTTTGCCC |
| | GGGCGGCCTCAGTGAGCGAGCGAGCGCGCAGAGAGGGAGTGGCCAA |
| | (SEQ ID NO: 448) |
| ITR (145 bp- | AGGAACCCCTAGTGATGGAGTTGGCCACTCCCTCTCTGCGCGCTCGCTCG |
| 1983, lacking | CTCACTGAGGCCGGGCGACCAAAGGTCGCCCGACGCCCGGGCTTTGCCC |
| 9 bp) | GGGCGGCCTCAGTGAGCGAGCGAGCGCGCAGAGAGGG (SEQ ID NO: 449) |

### II. Vectors and Virions

In one embodiment, the rAAV vector (also referred to as a rAAV virion) as disclosed herein comprises a capsid protein, and a rAAV genome in the capsid protein. A rAAV capsid of the rAAV virion used to treat Pompe Disease is any of those listed in Table 3 herein, or in Table 1 as disclosed in International Applications WO2020/102645, and WO2020/102667, each of which are incorporated herein in their entirety. In one embodiment, a rAAV capsid of the rAAV virion used to treat Pompe Disease is an AAV8 capsid. In one embodiment, a rAAV vector is an rAAV8 vector.

TABLE 3
Table 3: AAV Serotypes and exemplary Published corresponding capsid sequence

TABLE 3

| Serotype and where capsid sequence is published | Serotype and where capsid sequence is published |
|---|---|
| AAV3.3b (See SEQ ID NO: 72 in US20030138772) | AAV3-3 (See SEQ ID NO: 200 |
| | US20150315612) |
| AAV3-3 (See SEQ ID NO: 217 US20150315612) | AAV3a ((See SEQ ID NO: 5 in U.S. Pat. No. 6,156,303) |
| AAV3a (See SEQ ID NO: 9 in U.S. Pat. No. 6,156,303) | AAV3b (See SEQ ID NO: 6 in U.S. Pat. No. 6,156,303) |
| AAV3b (See SEQ ID NO: 10 in U.S. Pat. No. 6,156,303) | AAV3b (See SEQ ID NO: 1 in U.S. Pat. No. 6,156,303) |
| AAV4 (See SEQ ID NO: 17 US20140348794) | AAV4 ((See SEQ ID NO: 5 in US20140348794) |
| AAV4 (See SEQ ID NO: 3 in US20140348794) | AAV4 (See SEQ ID NO: 14 in |
| | US20140348794) |
| AAV4 (See SEQ ID NO: 15 in US20140348794) | AAV4 (See SEQ ID NO: 19 in |
| | US20140348794) |
| AAV4 (See SEQ ID NO: 12 in US20140348794) | AAV4 (See SEQ ID NO: 13 in |
| | US20140348794) |
| AAV4 (See SEQ ID NO: 7 in US20140348794) | AAV4 (See SEQ ID NO: 8 in |
| | US20140348794) |
| AAV4 (See SEQ ID NO: 9 in US20140348794) | AAV4 (See SEQ ID NO: 2 in |
| | US20140348794) |
| AAV4 (See SEQ ID NO: 10 in US20140348794) | AAV4 (See SEQ ID NO: 11 in |
| | US20140348794) |
| AAV4 (See SEQ ID NO: 18 in US20140348794) | AAV4 (See SEQ ID NO: 63 in |
| | US20030138772) and US20160017295 SEQ |
| ID NO: (See SEQ ID NO: 4 in US20140348794) | AAV4 (See SEQ ID NO: 16 in |
| | US20140348794) |
| AAV4 (See SEQ ID NO: 20 in US20140348794) | AAV4 (See SEQ ID NO: 6 in |
| | US20140348794) |
| AAV4 (See SEQ ID NO: 1 in US20140348794) | AAV42.2 (See SEQ ID NO: 9 in |
| | US20030138772) |
| AAV42.2 (See SEQ ID NO: 102 in | AAV42.3b (See SEQ ID NO: 36 in |
| US20030138772) | US20030138772) |
| AAV42.3B (See SEQ ID NO: 107 in | AAV42.4 (See SEQ ID NO: 33 in |
| US20030138772) | US20030138772) |
| AAV42.4 (See SEQ ID NO: 88 in | AAV42.8 (See SEQ ID NO: 27 in |
| US20030138772) | US20030138772) |
| AAV42.8 (See SEQ ID NO: 85 in | AAV43.1 (See SEQ ID NO: 39 in |
| US20030138772) | US20030138772) |
| AAV43.1 (See SEQ ID NO: 92 in | AAV43.12 (See SEQ ID NO: 41 in |
| US20030138772) | US20030138772) |
| AAV43.12 (See SEQ ID NO: 93 in | AAV8 (See SEQ ID NO: 15 in |
| US20030138772) | US20150159173) |
| AAV8 (See SEQ ID NO: 7 in US20150376240) | AAV8 (See SEQ ID NO: 4 in |
| | US20030138772; US20150315612 SEQ |
| ID NO: 182 | AAV8 (See SEQ ID NO: 95 in |
| | US20030138772), US20140359799 SEQ |
| AAV8 (See SEQ ID NO: 31 in US20150159173) | AAV8 (See, e.g., SEQ ID NO: 8 in |
| | US20160017295, or SEQ ID NO: 7 in |
| | U.S. Pat. No. 7,198,951, or SEQ ID NO: 223 in |
| | US20150315612) |
| AAV8 (See SEQ ID NO: 8 in US20150376240) | AAV8 (See SEQ ID NO: 214 in |
| | US20150315612) |
| AAV-8b (See SEQ ID NO: 5 in US20150376240) | AAV-8b (See SEQ ID NO: 3 in |
| | US20150376240) |
| AAV-8h (See SEQ ID NO: 6 in US20150376240) | AAV-8h (See SEQ ID NO: 4 in |
| | US20150376240) |
| AAV9 (See SEQ ID NO: 5 in US20030138772) | AAV9 (See SEQ ID NO: 1 in U.S. Pat. No. 7,198,951) |
| AAV9 (See SEQ ID NO: 9 in US20160017295) | AAV9 (See SEQ ID NO: 100 in |
| | US20030138772), U.S. Pat. No. 7198951 SEQ ID NO: 2 |
| | AAV9 (See SEQ ID NO: 3 in U.S. Pat. No. 7,198,951) |
| AAV9 (AAVhu.14) (See SEQ ID NO: 3 in | AAV9 (AAVhu.14) (See SEQ ID NO: 123 in |
| US20150315612) | US20150315612) |
| AAVA3.1 (See SEQ ID NO: 120 in | AAVA3.3 (See SEQ ID NO: 57 in |
| US20030138772) | US20030138772) |
| AAVA3.3 (See SEQ ID NO: 66 in | AAVA3.4 (See SEQ ID NO: 54 in |
| US20030138772) | US20030138772) |
| AAVA3.4 (See SEQ ID NO: 68 in | AAVA3.5 (See SEQ ID NO: 55 in |
| US20030138772) | US20030138772) |
| AAVA3.5 (See SEQ ID NO: 69 in | AAVA3.7 (See SEQ ID NO: 56 in |
| US20030138772) | US20030138772) |
| AAVA3.7 (See SEQ ID NO: 67 in | AAV29. (See SEQ ID NO: 11 in (AAVbb. 1) |
| US20030138772) | 161 US20030138772) |
| AAVC2 (See SEQ ID NO: 61 in US20030138772) | AAVCh.5 (See SEQ ID NO: 46 in |
| | US20150159173); US20150315612 SEQ |
| ID NO: 234 | AAVcy.2 (AAV13.3) (See SEQ ID NO: 15 in |
| | US20030138772) |
| AAV24.1 (See SEQ ID NO: 101 in | AAVcy.3 (AAV24.1) (See SEQ ID NO: 16 in |
| US20030138772) | US20030138772) |
| AAV27.3 (See SEQ ID NO: 104 in | AAVcy.4 (AAV27.3) (See SEQ ID NO: 17 in |
| US20030138772) | US20030138772) |
| AAVcy.5 (See SEQ ID NO: 227 in | AAV7.2 (See SEQ ID NO: 103 in |
| US20150315612) | US20030138772) |
| AAVcy.5 (AAV7.2) (See SEQ ID NO: 18 in | AAV16.3 (See SEQ ID NO: 105 in |
| US20030138772) | US20030138772) |
| AAVcy.6 (AAV16.3) (See SEQ ID NO: 10 in | AAVcy.5 (See SEQ ID NO: 8 in |
| US20030138772) | US20150159173) |
| AAVcy.5 (See SEQ ID NO: 24 in | AAVCy.5Rl (See SEQ ID NO: in |
| US20150159173) | US20150159173 |
| AAVCy.5R2 (See SEQ ID NO: in | AAVCy.5R3 (See SEQ ID NO: in |
| US20150159173) | US20150159173 |
| AAVCy.5R4 (See SEQ ID NO: in | AAVDJ (See SEQ ID NO: 3 in |
| US20150159173) | US20140359799) and SEQ ID NO: 2 in |
| | U.S. Pat. No. 7,588,772) |
| | AAVDJ (See SEQ ID NO: 2 in |
| | US20140359799; and SEQ ID NO: 1 in |
| | U.S. Pat. No. 7,588,772) |
| | AAVDJ-8 (See SEQ ID NO: in U.S. Pat. No. 7,588,772; |
| | Grimm et al 2008 |
| AAVDJ-8 (See SEQ ID NO: in U.S. Pat. No. 7,588,772; | AAVF5 (See SEQ ID NO: 110 in |
| Grimm et al 2008 | US20030138772) |
| AAVH2 (See SEQ ID NO: 26 in US20030138772) | AAVH6 (See SEQ ID NO: 25 in |
| | US20030138772) |
| AAVhEl. 1 (See SEQ ID NO: 44 in U.S. Pat. No. 9,233,131) | AAVhErl.14 (See SEQ ID NO: 46 in |
| | U.S. Pat. No. 9,233,131) |
| AAVhErl.16 (See SEQ ID NO: 48 in U.S. Pat. No. 9,233,131) | AAVhErl.18 (See SEQ ID NO: 49 in |
| | U.S. Pat. No. 9,233,131) |
| AAVhErl.23 (AAVhEr2.29) (See SEQ ID NO: 53 | AAVhErl.35 (See SEQ ID NO: 50 in |
| in U.S. Pat. No. 9,233,131) | U.S. Pat. No. 9,233,131) |
| AAVhErl.36 (See SEQ ID NO: 52 in U.S. Pat. No. 9,233,131) | AAVhEr1.5 (See SEQ ID NO: 45 in |
| | U.S. Pat. No. 9,233,131) |
| AAVhErl.7 (See SEQ ID NO: 51 in U.S. Pat. No. 9,233,131) | AAVhErl.8 (See SEQ ID NO: 47 in |
| | U.S. Pat. No. 9,233,131) |
| AAVhEr2.16 (See SEQ ID NO: 55 in U.S. Pat. No. 9,233,131) | AAVhEr2.30 (See SEQ ID NO: 56 in |
| | U.S. Pat. No. 9,233,131) |
| AAVhEr2.31 (See SEQ ID NO: 58 in U.S. Pat. No. 9,233,131) | AAVhEr2.36 (See SEQ ID NO: 57 in |
| | U.S. Pat. No. 9,233,131) |
| AAVhEr2.4 (See SEQ ID NO: 54 in U.S. Pat. No. 9,233,131) | AAVhEr3.1 (See SEQ ID NO: 59 in |
| | U.S. Pat. No. 9,233,131) |
| AAVhu.l (See SEQ ID NO: 46 in US20150315612) | AAVhu.l (See SEQ ID NO: 144 in |
| | US20150315612) |
| AAVhu.lO (AAV16.8) (See SEQ ID NO: 56 in | AAVhu.lO (AAV16.8) (See SEQ ID NO: 156 |
| US20150315612) | in US20150315612) |
| AAVhu.ll (AAV16.12) (See SEQ ID NO: 57 in | AAVhu.ll (AAV16.12) (See SEQ ID NO: 153 |
| US20150315612) | in US20150315612) |
| AAVhu.12 (See SEQ ID NO: 59 in | AAVhu.12 (See SEQ ID NO: 154 in |
| US20150315612) | US20150315612) |
| AAVhu.13 (See SEQ ID NO: 16 in | |
| US2015015917 and ID NO: 71 in US20150315612) | |
| AAVhu.13 (See SEQ ID NO: 32 in | |
| US20150159173 and ID NO: 129 US20150315612) | |
| AAVhu.136.1 (See SEQ ID NO: 165 in | AAVhu.140.1 (See SEQ ID NO: 166 in |
| US20150315612) | US20150315612) |
| AAVhu.140.2 (See SEQ ID NO: 167 in | AAVhu.145.6 (See SEQ ID NO: 178 in |
| US20150315612) | US20150315612) |
| AAVhu.15 (See SEQ ID NO: 147 in | AAVhu.15 (AAV33.4) (See SEQ ID NO: 50 in |
| US20150315612) | US20150315612) |
| AAVhu.156.1 (See SEQ ID NO: 179 in | AAVhu.16 (See SEQ ID NO: 148 in |
| US20150315612) | US20150315612) |
| AAVhu.l6 (AAV33.8) (See SEQ ID NO: 51 in | AAVhu.17 (See SEQ ID NO: 83 in |
| US20150315612) | US20150315612) |
| AAVhu.l7 (AAV33.12) (See SEQ ID NO: 4 in | AAVhu.172.1 (See SEQ ID NO: 171 in |
| US20150315612) | US20150315612) |
| AAVhu.172.2 (See SEQ ID NO: 172 in | AAVhu.173.4 (See SEQ ID NO: 173 in |
| US20150315612) | US20150315612) |
| AAVhu.173.8 (See SEQ ID NO: 175 in | AAVhu.18 (See SEQ ID NO: 52 in |
| US20150315612) | US20150315612) |
| AAVhu.18 (See SEQ ID NO: 149 in | AAVhu.19 (See SEQ ID NO: 62 in |
| US20150315612) | US20150315612) |
| AAVhu.19 (See SEQ ID NO: 133 in | AAVhu.2 (See SEQ ID NO: 48 in |
| US20150315612) | US20150315612) |
| AAVhu.2 (See SEQ ID NO: 143 in | AAVhu.20 (See SEQ ID NO: 63 in |
| US20150315612) | US20150315612) |
| AAVhu.20 (See SEQ ID NO: 134 in | AAVhu.21 (See SEQ ID NO: 65 in |
| US20150315612) | US20150315612) |
| AAVhu.21 (See SEQ ID NO: 135 in | AAVhu.22 (See SEQ ID NO: 67 in |
| US20150315612) | US20150315612) |
| AAVhu.22 239 (See SEQ ID NO: 138 in | AAVhu.23 (See SEQ ID NO: 60 in |
| US20150315612) | US20150315612) |
| AAVhu.23.2 (See SEQ ID NO: 137 in | AAVhu.24 (See SEQ ID NO: 66 in |
| US20150315612) | US20150315612) |
| AAVhu.24 (See SEQ ID NO: 136 in | AAVhu.25 (See SEQ ID NO: 49 in |
| US20150315612) | US20150315612) |
| AAVhu.25 (See SEQ ID NO: 146 in | AAVhu.26 (See SEQ ID NO: 17 in |
| US20150315612) | US20150159173 and SEQ ID NO: 61 in |
| | US20150315612) |
| | AAVhu.26 (See SEQ ID NO: 33 in |
| | US20150159173), US20150315612 SEQ |
| | AAVhu.27 (See SEQ ID NO: 64 in |
| | US20150315612) |
| AAVhu.27 (See SEQ ID NO: 140 in | AAVhu.28 (See SEQ ID NO: 68 in |
| US20150315612) | US20150315612) |
| AAVhu.28 (See SEQ ID NO: 130 in | AAVhu.29 (See SEQ ID NO: 69 in |
| US20150315612) | US20150315612) |
| AAVhu.29 (See SEQ ID NO: 42 in | |
| US20150159173 and SEQ ID NO: 132 in | |
| US20150315612) | |
| AAVhu.29 (See SEQ ID NO: 225 in | AAVhu.29R (See SEQ ID NO: in |
| US20150315612) | US20150159173 |
| AAVhu.3 (See SEQ ID NO: 44 in | AAVhu.3 (See SEQ ID NO: 145 in |
| US20150315612) | US20150315612) |
| AAVhu.30 (See SEQ ID NO: 70 in | AAVhu.30 (See SEQ ID NO: 131 in |
| US20150315612) | US20150315612) |
| AAVhu.31 (See SEQ ID NO: 1 in | AAVhu.31 (See SEQ ID NO: 121 in |
| US20150315612) | US20150315612) |
| AAVhu.32 (See SEQ ID NO: 2 in | AAVhu.32 (See SEQ ID NO: 122 in |
| US20150315612) | US20150315612) |
| AAVhu.33 (See SEQ ID NO: 75 in | AAVhu.33 (See SEQ ID NO: 124 in |
| US20150315612) | US20150315612) |
| AAVhu.34 (See SEQ ID NO: 72 in | AAVhu.34 (See SEQ ID NO: 125 in |
| US20150315612) | US20150315612) |
| AAVhu.35 (See SEQ ID NO: 73 in | AAVhu.35 (See SEQ ID NO: 164 in |
| US20150315612) | US20150315612) |
| AAVhu.36 (See SEQ ID NO: 74 in | AAVhu.36 (See SEQ ID NO: 126 in |
| US20150315612) | US20150315612) |
| AAVhu.37 (See SEQ ID NO: 34 in | |
| US20150159173 and SEQ ID NO: 88 in | |
| US20150315612) | |
| AAVhu.37 (AAV106.1) (See SEQ ID NO: 10 in | |
| US20150315612 and SEQ ID NO: 18 in | |
| US20150159173) | |
| AAVhu.38 (See SEQ ID NO: 161 in | AAVhu.39 (See SEQ ID NO: 102 in |
| US20150315612) | US20150315612) |
| AAVhu.39 (AAVLG-9) (See SEQ ID NO: 24 in | AAVhu.4 (See SEQ ID NO: 47 in |
| US20150315612) | US20150315612) |
| AAVhu.4 (See SEQ ID NO: 141 in | AAVhu.40 (See SEQ ID NO: 87 in |
| US20150315612) | US20150315612) |
| AAVhu.40 (AAV114.3) (See SEQ ID NO: 11 in | AAVhu.41 (See SEQ ID NO: 91 in |
| US20150315612) | US20150315612) |
| AAVhu.41 (AAV127.2) (See SEQ ID NO: 6 in | AAVhu.42 (See SEQ ID NO: 85 in |
| US20150315612) | US20150315612) |
| AAVhu.42 (AAV127.5) (See SEQ ID NO: 8 in | AAVhu.43 (See SEQ ID NO: 160 in |
| US20150315612) | US20150315612) |
| AAVhu.43 (See SEQ ID NO: 236 in | AAVhu.43 (AAV128.1) (See SEQ ID NO: 80 |
| US20150315612) | in US20150315612) |
| AAVhu.44 (See SEQ ID NO: 45 in | |
| US20150159173 and SEQ ID NO: 158 in | |
| US20150315612) | |
| AAVhu.44 (AAV128.3) (See SEQ ID NO: 81 in | AAVhu.44Rl (See SEQ ID NO: in |
| US20150315612) | US20150159173 |
| AAVhu.44R2 (See SEQ ID NO: in | AAVhu.44R3 (See SEQ ID NO: in |
| US20150159173 | US20150159173 |
| AAVhu.45 (See SEQ ID NO: 76 in | AAVhu.45 (See SEQ ID NO: 127 in |
| US20150315612) | US20150315612) |
| AAVhu.46 (See SEQ ID NO: 82 in | AAVhu.46 (See SEQ ID NO: 159 in |
| US20150315612) | US20150315612) |
| AAVhu.46 (See SEQ ID NO: 224 in | AAVhu.47 (See SEQ ID NO: 77 in |
| US20150315612) | US20150315612) |
| AAVhu.47 (See SEQ ID NO: 128 in | AAVhu.48 (See SEQ ID NO: 38 in |
| US20150315612) | US20150159173) |
| AAVhu.48 (See SEQ ID NO: 157 in | AAVhu.48 (AAV130.4) (See SEQ ID NO: 78 |
| US20150315612) | in US20150315612) |
| AAVhu.48Rl (See SEQ ID NO: in | AAVhu.48R2 (See SEQ ID NO: in |
| US20150159173 | US20150159173 |
| AAVhu.48R3 (See SEQ ID NO: in | AAVhu.49 (See SEQ ID NO: 209 in |
| US20150159173 | US20150315612) |
| AAVhu.49 (See SEQ ID NO: 189 in | AAVhu.5 (See SEQ ID NO: 45 in |
| US20150315612) | US20150315612) |
| AAVhu.5 (See SEQ ID NO: 142 in | AAVhu.51 (See SEQ ID NO: 208 in |
| US20150315612) | US20150315612) |
| AAVhu.51 (See SEQ ID NO: 190 in | AAVhu.52 (See SEQ ID NO: 210 in |
| US20150315612) | US20150315612) |
| AAVhu.52 (See SEQ ID NO: 191 in | AAVhu.53 (See SEQ ID NO: 19 in |
| US20150315612) | US20150159173) |
| AAVhu.53 (See SEQ ID NO: 35 in | AAVhu.53 (AAV145.1) (See SEQ ID NO: 176 |
| US20150159173) | in US20150315612) |
| AAVhu.54 (See SEQ ID NO: 188 in | AAVhu.54 (AAV145.5) (See SEQ ID NO: 177 |
| US20150315612) | in US20150315612) |
| AAVhu.55 (See SEQ ID NO: 187 in | AAVhu.56 (See SEQ ID NO: 205 in |
| US20150315612) | US20150315612) |
| AAVhu.56 (AAV145.6) (See SEQ ID NO: 168 in | AAVhu.56 (AAV145.6) (See SEQ ID NO: 192 |
| US20150315612) | in US20150315612) |
| AAVhu.57 (See SEQ ID NO: 206 in | AAVhu.57 (See SEQ ID NO: 169 in |
| US20150315612) | US20150315612) |
| AAVhu.57 (See SEQ ID NO: 193 in | AAVhu.58 (See SEQ ID NO: 207 in |
| US20150315612) | US20150315612) |
| AAVhu.58 (See SEQ ID NO: 194 in | AAVhu.6 (AAV3.1) (See SEQ ID NO: 5 in |
| US20150315612) | US20150315612) |
| AAVhu.6 (AAV3.1) (See SEQ ID NO: 84 in | AAVhu.60 (See SEQ ID NO: 184 in |
| US20150315612) | US20150315612) |
| AAVhu.60 (AAV161.10) (See SEQ ID NO: 170 in | AAVhu.61 (See SEQ ID NO: 185 in |
| US20150315612) | US20150315612) |
| AAVhu.61 (AAV161.6) (See SEQ ID NO: 174 in | AAVhu.63 (See SEQ ID NO: 204 in |
| US20150315612) | US20150315612) |
| AAVhu.63 (See SEQ ID NO: 195 in | AAVhu.64 (See SEQ ID NO: 212 in |
| US20150315612) | US20150315612) |
| AAVhu.64 (See SEQ ID NO: 196 in | AAVhu.66 (See SEQ ID NO: 197 in |
| US20150315612) | US20150315612) |
| AAVhu.67 (See SEQ ID NO: 215 in | AAVhu.67 (See SEQ ID NO: 198 in |
| US20150315612) | US20150315612) |
| AAVhu.7 (See SEQ ID NO: 226 in | AAVhu.7 (See SEQ ID NO: 150 in |
| US20150315612) | US20150315612) |
| AAVhu.7 (AAV7.3) (See SEQ ID NO: 55 in | AAVhu.71 (See SEQ ID NO: 79 in |
| US20150315612) | US20150315612) |
| AAVhu.8 (See SEQ ID NO: 53 in | AAVhu.8 (See SEQ ID NO: 12 in |
| US20150315612) | US20150315612) |
| AAVhu.8 (See SEQ ID NO: 151 in | AAVhu.9 (AAV3.1) (See SEQ ID NO: 58 in |
| US20150315612) | US20150315612) |
| AAVhu.9 (AAV3.1) (See SEQ ID NO: 155 in | AAV-LK01 (See SEQ ID NO: 2 in |
| US20150315612) | US20150376607) |
| AAV-LK01 (See SEQ ID NO: 29 in | AAV-LK02 (See SEQ ID NO: 3 in |
| US20150376607) | US20150376607) |
| AAV-LK02 (See SEQ ID NO: 30 in | AAV-LK03 (See SEQ ID NO: 4 in |
| US20150376607) | US20150376607) |
| AAV-LK03 (See SEQ ID NO: 12 in | |
| WO2015121501 and SEQ ID NO: 31 in | |
| US20150376607) | |
| AAV-LK04 (See SEQ ID NO: 5 in | AAV-LK04 (See SEQ ID NO: 32 in |
| US20150376607) | US20150376607) |
| AAV-LK05 (See SEQ ID NO: 6 in | AAV-LK05 (See SEQ ID NO: 33 in |
| US20150376607) | US20150376607) |
| AAV-LK06 (See SEQ ID NO: 7 in | AAV-LK06 (See SEQ ID NO: 34 in |
| US20150376607) | US20150376607) |
| AAV-LK07 (See SEQ ID NO: 8 in | AAV-LK07 (See SEQ ID NO: 35 in |
| US20150376607) | US20150376607) |
| AAV-LK08 (See SEQ ID NO: 9 in | AAV-LK08 (See SEQ ID NO: 36 in |
| US20150376607) | US20150376607) |
| AAV-LK09 (See SEQ ID NO: 10 in | AAV-LK09 (See SEQ ID NO: 37 in |
| US20150376607) | US20150376607) |
| AAV-LK10 (See SEQ ID NO: 11 in | AAV-LK10 (See SEQ ID NO: 38 in |
| US20150376607) | US20150376607) |
| AAV-LK11 (See SEQ ID NO: 12 in | AAV-LK11 (See SEQ ID NO: 39 in |
| US20150376607) | US20150376607) |
| AAV-LK12 (See SEQ ID NO: 13 in | AAV-LK12 (See SEQ ID NO: 40 in |
| US20150376607) | US20150376607) |
| AAV-LK13 (See SEQ ID NO: 14 in | AAV-LK13 (See SEQ ID NO: 41 in |
| US20150376607) | US20150376607) |
| AAV-LK14 (See SEQ ID NO: 15 in | AAV-LK14 (See SEQ ID NO: 42 in |
| US20150376607) | US20150376607) |
| AAV-LK15 (See SEQ ID NO: 16 in | AAV-LK15 (See SEQ ID NO: 43 in |
| US20150376607) | US20150376607) |
| AAV-LK16 (See SEQ ID NO: 17 in | AAV-LK16 (See SEQ ID NO: 44 in |
| US20150376607) | US20150376607) |
| AAV-LK17 (See SEQ ID NO: 18 in | AAV-LK17 (See SEQ ID NO: 45 in |
| US20150376607) | US20150376607) |
| AAV-LK18 (See SEQ ID NO: 19 in | AAV-LK18 (See SEQ ID NO: 46 in |
| US20150376607) | US20150376607) |
| AAV-LK19 (See SEQ ID NO: 20 in | AAV-LK19 (See SEQ ID NO: 47 in |
| US20150376607) | US20150376607) |
| AAV-PAEC (See SEQ ID NO: 1 in | AAV-PAEC (See SEQ ID NO: 48 in |
| US20150376607) | US20150376607) |
| AAV-PAEC11 (See SEQ ID NO: 26 in | AAV-PAEC11 (See SEQ ID NO: 54 in |
| US20150376607) | US20150376607) |
| AAV-PAEC 12 (See SEQ ID NO: 27 in | AAV-PAEC 12 (See SEQ ID NO: 51 in |
| US20150376607) | US20150376607) |
| AAV-PAEC 13 (See SEQ ID NO: 28 in | AAV-PAEC 13 (See SEQ ID NO: 49 in |
| US20150376607) | US20150376607) |
| AAV-PAEC2 (See SEQ ID NO: 21 in | AAV-PAEC2 (See SEQ ID NO: 56 in |
| US20150376607) | US20150376607) |
| AAV-PAEC4 (See SEQ ID NO: 22 in | AAV-PAEC4 (See SEQ ID NO: 55 in |
| US20150376607) | US20150376607) |
| AAV-PAEC6 (See SEQ ID NO: 23 in | AAV-PAEC6 (See SEQ ID NO: 52 in |
| US20150376607) | US20150376607) |
| AAV-PAEC7 (See SEQ ID NO: 24 in | AAV-PAEC7 (See SEQ ID NO: 53 in |
| US20150376607) | US20150376607) |
| AAV-PAEC8 (See SEQ ID NO: 25 in | AAV-PAEC8 (See SEQ ID NO: 50 in |
| US20150376607) | US20150376607) |
| AAVpi.l (See SEQ ID NO: 28 in US20150315612) | AAVpi.l (See SEQ ID NO: 93 in |
| | US20150315612; AAVpi.2 408, see SEQ ID |
| | NO: 30 in US20150315612) |
| AAVpi.2 (See SEQ ID NO: 95 in | AAVpi.3 (See SEQ ID NO: 29 in |
| US20150315612) | US20150315612) |
| AAVpi.3 (See SEQ ID NO: 94 in | AAVrh.10 (See SEQ ID NO: 9 in |
| US20150315612) | US20150159173) |
| AAVrh.10 (See SEQ ID NO: 25 in | AAV44.2 (See SEQ ID NO: 59 in |
| US20150159173) | US20030138772) |
| AAVrh.10 (AAV44.2) (See SEQ ID NO: 81 in | AAV42.1B (See SEQ ID NO: 90 in |
| US20030138772) | US20030138772) |
| AAVrh.l2 (AAV42.1b) (See SEQ ID NO: 30 in | AAVrh.13 (See SEQ ID NO: 10 in |
| US20030138772) | US20150159173) |
| AAVrh.13 (See SEQ ID NO: 26 in | AAVrh.13 (See SEQ ID NO: 228 in |
| US20150159173) | US20150315612) |
| AAVrh.l3R (See SEQ ID NO: in US20150159173 | AAV42.3A (See SEQ ID NO: 87 in |
| | US20030138772) |
| AAVrh.l4 (AAV42.3a) (See SEQ ID NO: 32 in | AAV42.5A (See SEQ ID NO: 89 in |
| US20030138772) | US20030138772) |
| AAVrh.l7 (AAV42.5a) (See SEQ ID NO: 34 in | AAV42.5B (See SEQ ID NO: 91 in |
| US20030138772) | US20030138772) |
| AAVrh.l8 (AAV42.5b) (See SEQ ID NO: 29 in | AAV42.6B (See SEQ ID NO: 112 in |
| US20030138772) | US20030138772) |
| AAVrh.l9 (AAV42.6b) (See SEQ ID NO: 38 in | AAVrh.2 (See SEQ ID NO: 39 in |
| US20030138772) | US20150159173) |
| AAVrh.2 (See SEQ ID NO: 231 in | AAVrh.20 (See SEQ ID NO: 1 in |
| US20150315612) | US20150159173) |
| AAV42.10 (See SEQ ID NO: 106 in | AAVrh.21 (AAV42.10) (See SEQ ID NO: 35 |
| US20030138772) | in US20030138772) |
| AAV42.11 (See SEQ ID NO: 108 in | AAVrh.22 (AAV42.11) (See SEQ ID NO: 37 |
| US20030138772) | in US20030138772) |
| AAV42.12 (See SEQ ID NO: 113 in | AAVrh.23 (AAV42.12) (See SEQ ID NO: 58 |
| US20030138772) | in US20030138772) |
| AAV42.13 (See SEQ ID NO: 86 in | AAVrh.24 (AAV42.13) (See SEQ ID NO: 31 |
| US20030138772) | in US20030138772) |
| AAV42.15 (See SEQ ID NO: 84 in | AAVrh.25 (AAV42.15) (See SEQ ID NO: 28 |
| US20030138772) | in US20030138772) |
| AAVrh.2R (See SEQ ID NO: in US20150159173 | AAVrh.31 (AAV223.1) (See SEQ ID NO: 48 |
| | in US20030138772) |
| AAVC1 (See SEQ ID NO: 60 in US20030138772) | AAVrh.32 (AAVC1) (See SEQ ID NO: 19 in |
| | 446 US20030138772) |
| AAVrh.32/33 (See SEQ ID NO: 2 in | AAVrh.51 (AAV2-5) (See SEQ ID NO: 104 in |
| US20150159173) | US20150315612) |
| AAVrh.52 (AAV3-9) (See SEQ ID NO: 18 in | AAVrh.52 (AAV3-9) (See SEQ ID NO: 96 in |
| US20150315612) | US20150315612) |
| AAVrh.53 (See SEQ ID NO: in US20150315612) | AAVrh.53 (AAV3-11) (See SEQ ID NO: 17 in |
| | US20150315612) |
| AAVrh.53 (AAV3-11) (See SEQ ID NO: 186 in | AAVrh.54 (See SEQ ID NO: 40 in |
| US20150315612) | US20150315612) |
| AAVrh.54 (See SEQ ID NO: 49 in | |
| US20150159173 and SEQ ID NO: 116 in | |
| US20150315612) | |
| AAVrh.55 (See SEQ ID NO: 37 in | AAVrh.55 (AAV4-19) (See SEQ ID NO: 117 |
| US20150315612) | in US20150315612) |
| AAVrh.56 (See SEQ ID NO: 54 in | AAVrh.56 (See SEQ ID NO: 152 in |
| US20150315612) | US20150315612) |
| AAVrh.57 (See SEQ ID NO: in 497 | AAVrh.57 (See SEQ ID NO: 105 in |
| US20150315612 SEQ ID NO: 26 | US20150315612) |
| AAVrh.58 (See SEQ ID NO: 27 in | AAVrh.58 (See SEQ ID NO: 48 in |
| US20150315612) | US20150159173 and SEQ ID NO: 106 in |
| | US20150315612) |
| | AAVrh.58 (See SEQ ID NO: 232 in |
| | US20150315612) |
| AAVrh.59 (See SEQ ID NO: 42 in | AAVrh.59 (See SEQ ID NO: 110 in |
| US20150315612) | US20150315612) |
| AAVrh.60 (See SEQ ID NO: 31 in | AAVrh.60 (See SEQ ID NO: 120 in |
| US20150315612) | US20150315612) |
| AAVrh.61 (See SEQ ID NO: 107 in | AAVrh.61 (AAV2-3) (See SEQ ID NO: 21 in |
| US20150315612) | US20150315612) |
| AAVrh.62 (AAV2-15) (See SEQ ID NO: 33 in | AAVrh.62 (AAV2-15) (See SEQ ID NO: 114 |
| US20150315612) | in US20150315612) |
| AAVrh.64 (See SEQ ID NO: 15 in | AAVrh.64 (See SEQ ID NO: 43 in |
| US20150315612) | US20150159173 and SEQ ID NO: 99 in |
| | US20150315612) |
| | AAVrh.64 (See SEQ ID NO: 233 in |
| | US20150315612) |
| AAVRh.64Rl (See SEQ ID NO: in | AAVRh.64R2 (See SEQ ID NO: in |
| US20150159173 | US20150159173 |
| AAVrh.65 (See SEQ ID NO: 35 in | AAVrh.65 (See SEQ ID NO: 112 in |
| US20150315612) | US20150315612) |
| AAVrh.67 (See SEQ ID NO: 36 in | AAVrh.67 (See SEQ ID NO: 230 in |
| US20150315612) | US20150315612) |
| AAVrh.67 (See SEQ ID NO: 47 in | |
| US20150159173 and SEQ ID NO: 47 in | |
| US20150315612) | |
| AAVrh.68 (See SEQ ID NO: 16 in | AAVrh.68 (See SEQ ID NO: 100 in |
| US20150315612) | US20150315612) |
| AAVrh.69 (See SEQ ID NO: 39 in | AAVrh.69 (See SEQ ID NO: 119 in |
| US20150315612) | US20150315612) |
| AAVrh.70 (See SEQ ID NO: 20 in | AAVrh.70 (See SEQ ID NO: 98 in |
| US20150315612) | US20150315612) |
| AAVrh.71 (See SEQ ID NO: 162 in | AAVrh.72 (See SEQ ID NO: 9 in |
| US20150315612) | US20150315612) |
| AAVrh.73 (See SEQ ID NO: 5 in | AAVrh.74 (See SEQ ID NO: 6 in |
| US20150159173) | US20150159173) |
| AAVrh.8 (See SEQ ID NO: 41 in | AAVrh.8 (See SEQ ID NO: 235 in |
| US20150159173) | US20150315612) |
| AAVrh.8R (See SEQ ID NO: 9 in | AAVrh.8R A586R mutant (See SEQ ID NO: 10 |
| US20150159173, WO2015168666) | in WO2015168666) |
| AAVrh.8R R533A mutant (See SEQ ID NO: 11 in | BAAV (bovine AAV) (See SEQ ID NO: 8 in |
| WO2015168666) | U.S. Pat. No. 9,193,769) |
| BAAV (bovine AAV) (See SEQ ID NO: 10 in | BAAV (bovine AAV) (See SEQ ID NO: 4 in |
| U.S. Pat. No. 9,193,769) | U.S. Pat. No. 9,193,769) |
| BAAV (bovine AAV) (See SEQ ID NO: 2 in | BAAV (bovine AAV) (See SEQ ID NO: 6 in |
| U.S. Pat. No. 9,193,769) | U.S. Pat. No. 9,193,769) |
| BAAV (bovine AAV) (See SEQ ID NO: 1 in | BAAV (bovine AAV) (See SEQ ID NO: 5 in |
| U.S. Pat. No. 9,193,769) | U.S. Pat. No. 9,193,769) |
| BAAV (bovine AAV) (See SEQ ID NO: 3 in | BAAV (bovine AAV) (See SEQ ID NO: 11 in |
| U.S. Pat. No. 9,193,769) | U.S. Pat. No. 9,193,769) |
| BAAV (bovine AAV) (See SEQ ID NO: 5 in | BAAV (bovine AAV) (See SEQ ID NO: 6 in |
| U.S. Pat. No. 7,427,396) | U.S. Pat. No. 7,427,396) |
| BAAV (bovine AAV) (See SEQ ID NO: 7 in | BAAV (bovine AAV) (See SEQ ID NO: 9 in |
| U.S. Pat. No. 9,193,769) | U.S. Pat. No. 9,193,769) |
| BNP61 AAV (See SEQ ID NO: 1 in | BNP61 AAV (See SEQ ID NO: 2 in |
| US20150238550) | US20150238550) |
| BNP62 AAV (See SEQ ID NO: 3 in | BNP63 AAV (See SEQ ID NO: 4 in |
| US20150238550) | US20150238550) |
| caprine AAV (See SEQ ID NO: 3 in U.S. Pat. No. 7,427,396) | caprine AAV (See SEQ ID NO: 4 in |
| | U.S. Pat. No. 7,427,396) |
| true type AAV (ttAAV) (See SEQ ID NO: 2 in | AAAV (Avian AAV) (See SEQ ID NO: 12 in |
| WO2015121501) | U.S. Pat. No. 9,238,800) |
| AAAV (Avian AAV) (See SEQ ID NO: 2 in | AAAV (Avian AAV) (See SEQ ID NO: 6 in |
| U.S. Pat. No. 9,238,800) | U.S. Pat. No. 9,238,800) |
| AAAV (Avian AAV) (See SEQ ID NO: 4 in | AAAV (Avian AAV) (See SEQ ID NO: 8 in |
| U.S. Pat. No. 9,238,800) | U.S. Pat. No. 9,238,800) |
| AAAV (Avian AAV) (See SEQ ID NO: 14 in | AAAV (Avian AAV) (See SEQ ID NO: 10 in |
| U.S. Pat. No. 9,238,800) | U.S. Pat. No. 9,238,800) |
| AAAV (Avian AAV) (See SEQ ID NO: 15 in | AAAV (Avian AAV) (See SEQ ID NO: 5 in |
| U.S. Pat. No. 9,238,800) | U.S. Pat. No. 9,238,800) |
| AAAV (Avian AAV) (See SEQ ID NO: 9 in | AAAV (Avian AAV) (See SEQ ID NO: 3 in |
| U.S. Pat. No. 9,238,800) | U.S. Pat. No. 9,238,800) |
| AAAV (Avian AAV) (See SEQ ID NO: 7 in | AAAV (Avian AAV) (See SEQ ID NO: 11 in |
| U.S. Pat. No. 9,238,800) | U.S. Pat. No. 9,238,800) |
| AAAV (Avian AAV) (See SEQ ID NO: in | AAAV (Avian AAV) (See SEQ ID NO: 1 in |
| U.S. Pat. No. 9,238,800) | U.S. Pat. No. 9,238,800) |
| AAV Shuffle 100-1 (See SEQ ID NO: 23 in | AAV Shuffle 100-1 (See SEQ ID NO: 11 in |
| US20160017295) | US20160017295) |
| AAV Shuffle 100-2 (See SEQ ID NO: 37 in | AAV Shuffle 100-2 (See SEQ ID NO: 29 in |
| US20160017295) | US20160017295) |
| AAV Shuffle 100-3 (See SEQ ID NO: 24 in | AAV Shuffle 100-3 (See SEQ ID NO: 12 in |
| US20160017295) | US20160017295) |
| AAV Shuffle 100-7 (See SEQ ID NO: 25 in | AAV Shuffle 100-7 (See SEQ ID NO: 13 in |
| US20160017295) | US20160017295) |
| AAV Shuffle 10-2 (See SEQ ID NO: 34 in | AAV Shuffle 10-2 (See SEQ ID NO: 26 in |
| US20160017295) | US20160017295) |
| AAV Shuffle 10-6 (See SEQ ID NO: 35 in | AAV Shuffle 10-6 (See SEQ ID NO: 27 in |
| US20160017295) | US20160017295) |
| AAV Shuffle 10-8 (See SEQ ID NO: 36 in | AAV Shuffle 10-8 (See SEQ ID NO: 28 in |
| US20160017295) | US20160017295) |
| AAV SM 100-10 (See SEQ ID NO: 41 in | AAV SM 100-10 (See SEQ ID NO: 33 in |
| US20160017295) | US20160017295) |
| AAV SM 100-3 (See SEQ ID NO: 40 in | AAV SM 100-3 (See SEQ ID NO: 32 in |
| US20160017295) | US20160017295) |
| AAV SM 10-1 (See SEQ ID NO: 38 in | AAV SM 10-1 (See SEQ ID NO: 30 in |
| US20160017295) | US20160017295) |
| AAV SM 10-2 (See SEQ ID NO: 10 in | AAV SM 10-2 (See SEQ ID NO: 22 in |
| US20160017295) | US20160017295) |
| AAV SM 10-8 (See SEQ ID NO: 39 in | AAV SM 10-8 (See SEQ ID NO: 31 in |
| US20160017295) | US20160017295) |
| AAV CBr-7.1 (See SEQ ID NO: 4 in | AAV CBr-7.1 (See SEQ ID NO: 54 in |
| WO2016065001) | WO2016065001) |
| AAV CBr-7.10 (See SEQ ID NO: 11 in | AAV CBr-7.10 (See SEQ ID NO: 61 in |
| WO2016065001) | WO2016065001) |
| AAV CBr-7.2 (See SEQ ID NO: 5 in | AAV CBr-7.2 (See SEQ ID NO: 55 in |
| WO2016065001) | WO2016065001) |
| AAV CBr-7.3 (See SEQ ID NO: 6 in | AAV CBr-7.3 (See SEQ ID NO: 56 in |
| WO2016065001) | WO2016065001) |
| AAV CBr-7.4 (See SEQ ID NO: 7 in | AAV CBr-7.4 (See SEQ ID NO: 57 in |
| WO2016065001) | WO2016065001) |
| AAV CBr-7.5 (See SEQ ID NO: 8 in | AAV CHt-6.6 (See SEQ ID NO: 35 in |
| WO2016065001) | WO2016065001) |
| AAV CHt-6.6 (See SEQ ID NO: 85 in | AAV CHt-6.7 (See SEQ ID NO: 36 in |
| WO2016065001) | WO2016065001) |
| AAV CHt-6.7 (See SEQ ID NO: 86 in | AAV CHt-6.8 (See SEQ ID NO: 37 in |
| WO2016065001) | WO2016065001) |
| AAV CHt-6.8 (See SEQ ID NO: 87 in | AAV CHt-Pl (See SEQ ID NO: 29 in |
| WO2016065001) | WO2016065001) |
| AAV CHt-Pl (See SEQ ID NO: 79 in | AAV CHt-P2 (See SEQ ID NO: 1 in |
| WO2016065001) | WO2016065001) |
| AAV CHt-P2 (See SEQ ID NO: 51 in | AAV CHt-P5 (See SEQ ID NO: 2 in |
| WO2016065001) | WO2016065001) |
| AAV CHt-P5 (See SEQ ID NO: 52 in | AAV CHt-P6 (See SEQ ID NO: 30 in |
| WO2016065001) | WO2016065001) |
| AAV CHt-P6 (See SEQ ID NO: 80 in | AAV CHt-P8 (See SEQ ID NO: 31 in |
| WO2016065001) | WO2016065001) |
| AAV CHt-P8 (See SEQ ID NO: 81 in | AAV CHt-P9 (See SEQ ID NO: 3 in |
| WO2016065001) | WO2016065001) |
| AAV CHt-P9 (See SEQ ID NO: 53 in | AAV CKd-1 (See SEQ ID NO: 57 in |
| WO2016065001) | U.S. Pat. No. 8,734,809) |
| AAV CKd-1 (See SEQ ID NO: 131 in | AAV CKd-10 (See SEQ ID NO: 58 in |
| U.S. Pat. No. 8,734,809) | U.S. Pat. No. 8,734,809) |
| AAV CKd-10 (See SEQ ID NO: 132 in | AAV CKd-2 (See SEQ ID NO: 59 in |
| U.S. Pat. No. 8,734,809) | U.S. Pat. No. 8,734,809) |
| AAV CKd-2 (See SEQ ID NO: 133 in | AAV CKd-3 (See SEQ ID NO: 60 in |
| U.S. Pat. No. 8,734,809) | U.S. Pat. No. 8,734,809) |
| AAV CKd-3 (See SEQ ID NO: 134 in | AAV CKd-4 (See SEQ ID NO: 61 in |
| U.S. Pat. No. 8,734,809) | U.S. Pat. No. 8,734,809) |
| AAV CKd-4 (See SEQ ID NO: 135 in | AAV CKd-6 (See SEQ ID NO: 62 in |
| U.S. Pat. No. 8,734,809) | U.S. Pat. No. 8,734,809) |
| AAV CKd-6 (See SEQ ID NO: 136 in | AAV CKd-7 (See SEQ ID NO: 63 in |
| U.S. Pat. No. 8,734,809) | U.S. Pat. No. 8,734,809) |
| AAV CKd-7 (See SEQ ID NO: 137 in | AAV CKd-8 (See SEQ ID NO: 64 in |
| U.S. Pat. No. 8,734,809) | U.S. Pat. No. 8,734,809) |
| AAV CKd-8 (See SEQ ID NO: 138 in | AAV CKd-B 1 (See SEQ ID NO: 73 in |
| U.S. Pat. No. 8,734,809) | U.S. Pat. No. 8,734,809) |
| AAV CKd-B 1 (See SEQ ID NO: 147 in | AAV CKd-B2 (See SEQ ID NO: 74 in |
| U.S. Pat. No. 8,734,809) | U.S. Pat. No. 8,734,809) |
| AAV CKd-B2 (See SEQ ID NO: 148 in | AAV CKd-B3 (See SEQ ID NO: 75 in |
| U.S. Pat. No. 8,734,809) | U.S. Pat. No. 8,734,809) |
| AAV CKd-B3 (See SEQ ID NO: in U.S. Pat. No. 8,734,809 | AAV CKd-B3 (See SEQ ID NO: 149 in |
| | U.S. Pat. No. 8,734,809) |
| AAV CLv-1 (See SEQ ID NO: 65 in U.S. Pat. No. 8,734,809) | AAV CLv-1 (See SEQ ID NO: 139 in |
| | U.S. Pat. No. 8,734,809) |
| AAV CLvl-1 (See SEQ ID NO: 171 in | AAV Civ 1-10 (See SEQ ID NO: 178 in |
| U.S. Pat. No. 8,734,809) | U.S. Pat. No. 8,734,809) |
| AAV CLvl-2 (See SEQ ID NO: 172 in | AAV CLv-12 (See SEQ ID NO: 66 in |
| U.S. Pat. No. 8,734,809) | U.S. Pat. No. 8,734,809) |
| AAV CLv-12 (See SEQ ID NO: 140 in | AAV CLvl-3 (See SEQ ID NO: 173 in |
| U.S. Pat. No. 8,734,809) | U.S. Pat. No. 8,734,809) |
| AAV CLv-13 (See SEQ ID NO: 67 in | AAV CLv-13 (See SEQ ID NO: 141 in |
| U.S. Pat. No. 8,734,809) | U.S. Pat. No. 8,734,809) |
| AAV CLvl-4 (See SEQ ID NO: 174 in | AAV Civ 1-7 (See SEQ ID NO: 175 in |
| U.S. Pat. No. 8,734,809) | U.S. Pat. No. 8,734,809) |
| AAV Civ 1-8 (See SEQ ID NO: 176 in | AAV Civ 1-9 (See SEQ ID NO: 177 in |
| U.S. Pat. No. 8,734,809) | U.S. Pat. No. 8,734,809) |
| AAV CLv-2 (See SEQ ID NO: 68 in U.S. Pat. No. 8,734,809) | AAV CLv-2 (See SEQ ID NO: 142 in |
| | U.S. Pat. No. 8,734,809) |
| AAV CLv-3 (See SEQ ID NO: 69 in U.S. Pat. No. 8,734,809) | AAV CLv-3 (See SEQ ID NO: 143 in |
| | U.S. Pat. No. 8,734,809) |
| AAV CLv-4 (See SEQ ID NO: 70 in U.S. Pat. No. 8,734,809) | AAV CLv-4 (See SEQ ID NO: 144 in |
| | U.S. Pat. No. 8,734,809) |
| AAV CLv-6 (See SEQ ID NO: 71 in U.S. Pat. No. 8,734,809) | AAV CLv-6 (See SEQ ID NO: 145 in |
| | U.S. Pat. No. 8,734,809) |
| AAV CLv-8 (See SEQ ID NO: 72 in U.S. Pat. No. 8,734,809) | AAV CLv-8 (See SEQ ID NO: 146 in |
| | U.S. Pat. No. 8,734,809) |
| AAV CLv-Dl (See SEQ ID NO: 22 in | AAV CLv-Dl (See SEQ ID NO: 96 in |
| U.S. Pat. No. 8,734,809) | U.S. Pat. No. 8,734,809) |
| AAV CLv-D2 (See SEQ ID NO: 23 in | AAV CLv-D2 (See SEQ ID NO: 97 in |
| U.S. Pat. No. 8,734,809) | U.S. Pat. No. 8,734,809) |
| AAV CLv-D3 (See SEQ ID NO: 24 in | AAV CLv-D3 (See SEQ ID NO: 98 in |
| U.S. Pat. No. 8,734,809) | U.S. Pat. No. 8,734,809) |
| AAV CLv-D4 (See SEQ ID NO: 25 in | AAV CLv-D4 (See SEQ ID NO: 99 in |
| U.S. Pat. No. 8,734,809) | U.S. Pat. No. 8,734,809) |
| AAV CLv-D5 (See SEQ ID NO: 26 in | AAV CLv-D5 (See SEQ ID NO: 100 in |
| U.S. Pat. No. 8,734,809) | U.S. Pat. No. 8,734,809) |
| AAV CLv-D6 (See SEQ ID NO: 27 in | AAV CLv-D6 (See SEQ ID NO: 101 in |
| U.S. Pat. No. 8,734,809) | U.S. Pat. No. 8,734,809) |
| AAV CLv-D7 (See SEQ ID NO: 28 in | AAV CLv-D7 (See SEQ ID NO: 102 in |
| U.S. Pat. No. 8,734,809) | U.S. Pat. No. 8,734,809) |
| AAV CLv-D8 (See SEQ ID NO: 29 in | AAV CLv-D8 (See SEQ ID NO: 103 in |
| U.S. Pat. No. 8,734,809) | U.S. Pat. No. 8734809); AAV CLv-Kl 762, see SEQ ID |
| | NO: 18 in WO2016065001) |
| AAV CLv-Kl (See SEQ ID NO: 68 in | AAV CLv-K3 (See SEQ ID NO: 19 in |
| WO2016065001) | WO2016065001) |
| AAV CLv-K3 (See SEQ ID NO: 69 in | AAV CLv-K6 (See SEQ ID NO: 20 in |
| WO2016065001) | WO2016065001) |
| AAV CLv-K6 (See SEQ ID NO: 70 in | AAV CLv-L4 (See SEQ ID NO: 15 in |
| WO2016065001) | WO2016065001) |
| AAV CLv-L4 (See SEQ ID NO: 65 in | AAV CLv-L5 (See SEQ ID NO: 16 in |
| WO2016065001) | WO2016065001) |
| AAV CLv-L5 (See SEQ ID NO: 66 in | AAV CLv-L6 (See SEQ ID NO: 17 in |
| WO2016065001) | WO2016065001) |
| AAV CLv-L6 (See SEQ ID NO: 67 in | AAV CLv-Ml (See SEQ ID NO: 21 in |
| WO2016065001) | WO2016065001) |
| AAV CLv-Ml (See SEQ ID NO: 71 in | AAV CLv-Mll (See SEQ ID NO: 22 in |
| WO2016065001) | WO2016065001) |
| AAV CLv-Ml 1 (See SEQ ID NO: 72 in | AAV CLv-M2 (See SEQ ID NO: 23 in |
| WO2016065001) | WO2016065001) |
| AAV CLv-M2 (See SEQ ID NO: 73 in | AAV CLv-M5 (See SEQ ID NO: 24 in |
| WO2016065001) | WO2016065001) |
| AAV CLv-M5 (See SEQ ID NO: 74 in | AAV CLv-M6 (See SEQ ID NO: 25 in |
| WO2016065001) | WO2016065001) |
| AAV CLv-M6 (See SEQ ID NO: 75 in | AAV CLv-M7 (See SEQ ID NO: 26 in |
| WO2016065001) | WO2016065001) |
| AAV CLv-M7 (See SEQ ID NO: 76 in | AAV CLv-M8 (See SEQ ID NO: 27 in |
| WO2016065001) | WO2016065001) |
| AAV CLv-M8 (See SEQ ID NO: 77 in | AAV CLv-M9 (See SEQ ID NO: 28 in |
| WO2016065001) | WO2016065001) |
| AAV CLv-M9 (See SEQ ID NO: 78 in | AAV CLv-Rl (See SEQ ID NO: 30 in |
| WO2016065001) | U.S. Pat. No. 8,734,809) |
| AAV CLv-Rl (See SEQ ID NO: 104 in | AAV CLv-R2 (See SEQ ID NO: 31 in |
| U.S. Pat. No. 8,734,809) | U.S. Pat. No. 8,734,809) |
| AAV CLv-R2 (See SEQ ID NO: 105 in | AAV CLv-R3 (See SEQ ID NO: 32 in |
| U.S. Pat. No. 8,734,809) | U.S. Pat. No. 8,734,809) |
| AAV CLv-R3 (See SEQ ID NO: 106 in | AAV CLv-R4 (See SEQ ID NO: 33 in |
| U.S. Pat. No. 8,734,809) | U.S. Pat. No. 8,734,809) |
| AAV CLv-R4 (See SEQ ID NO: 107 in | AAV CLv-R5 (See SEQ ID NO: 34 in |
| U.S. Pat. No. 8,734,809) | U.S. Pat. No. 8,734,809) |
| AAV CLv-R5 (See SEQ ID NO: 108 in | AAV CLv-R6 (See SEQ ID NO: 35 in |
| U.S. Pat. No. 8,734,809) | U.S. Pat. No. 8,734,809) |
| AAV CLv-R6 (See SEQ ID NO: 109 in | AAV CLv-R7 (See SEQ ID NO: 110 in |
| U.S. Pat. No. 8734809); AAV CLv-R7 802 (see SEQ ID NO: | U.S. Pat. No. 8,734,809) |
| 36 in U.S. Pat. No. 8,734,809) | |
| AAV CLv-R8 (See SEQ ID NO: 37 in | AAV CLv-R8 (See SEQ ID NO: 111 in |
| U.S. Pat. No. 8,734,809) | U.S. Pat. No. 8,734,809) |
| AAV CLv-R9 (See SEQ ID NO: 38 in | AAV CLv-R9 (See SEQ ID NO: 112 in |
| U.S. Pat. No. 8,734,809) | U.S. Pat. No. 8,734,809) |
| AAV CSp-1 (See SEQ ID NO: 45 in U.S. Pat. No. 8,734,809) | AAV CSp-1 (See SEQ ID NO: 119 in |
| | U.S. Pat. No. 8,734,809) |
| AAV CSp-10 (See SEQ ID NO: 46 in U.S. Pat. No. 8,734,809) | AAV CSp-10 (See SEQ ID NO: 120 in |
| | U.S. Pat. No. 8,734,809) |
| AAV CSp-11 (See SEQ ID NO: 47 in U.S. Pat. No. 8,734,809) | AAV CSp-11 (See SEQ ID NO: 121 in |
| | U.S. Pat. No. 8,734,809) |
| AAV CSp-2 (See SEQ ID NO: 48 in U.S. Pat. No. 8,734,809) | AAV CSp-2 (See SEQ ID NO: 122 in |
| | U.S. Pat. No. 8,734,809) |
| AAV CSp-3 (See SEQ ID NO: 49 in U.S. Pat. No. 8,734,809) | AAV CSp-3 (See SEQ ID NO: 123 in |
| | U.S. Pat. No. 8,734,809) |
| AAV CSp-4 (See SEQ ID NO: 50 in U.S. Pat. No. 8,734,809) | AAV CSp-4 (See SEQ ID NO: 124 in |
| | U.S. Pat. No. 8,734,809) |
| AAV CSp-6 (See SEQ ID NO: 51 in U.S. Pat. No. 8,734,809) | AAV CSp-6 (See SEQ ID NO: 125 in |
| | U.S. Pat. No. 8,734,809) |
| AAV CSp-7 (See SEQ ID NO: 52 in U.S. Pat. No. 8,734,809) | AAV CSp-7 (See SEQ ID NO: 126 in |
| | U.S. Pat. No. 8,734,809) |
| AAV CSp-8 (See SEQ ID NO: 53 in U.S. Pat. No. 8,734,809) | AAV CSp-8 (See SEQ ID NO: 127 in |
| | U.S. Pat. No. 8,734,809) |
| AAV CSp-8.10 (See SEQ ID NO: 38 in | AAV CSp-8.10 (See SEQ ID NO: 88 in |
| WO2016065001) | WO2016065001) |
| AAV CSp-8.2 (See SEQ ID NO: 39 in | AAV CSp-8.2 (See SEQ ID NO: 89 in |
| WO2016065001) | WO2016065001) |
| AAV CSp-8.4 (See SEQ ID NO: 40 in | AAV CSp-8.4 (See SEQ ID NO: 90 in |
| WO2016065001) | WO2016065001) |
| AAV CSp-8.5 (See SEQ ID NO: 41 in | AAV CSp-8.5 (See SEQ ID NO: 91 in |
| WO2016065001) | WO2016065001) |
| AAV CSp-8.6 (See SEQ ID NO: 42 in | AAV CSp-8.6 (See SEQ ID NO: 92 in |
| WO2016065001) | WO2016065001) |
| AAV CSp-8.7 (See SEQ ID NO: 43 in | AAV CSp-8.7 (See SEQ ID NO: 93 in |
| WO2016065001) | WO2016065001) |
| AAV CSp-8.8 (See SEQ ID NO: 44 in | AAV CSp-8.8 (See SEQ ID NO: 94 in |
| WO2016065001) | WO2016065001) |
| AAV CSp-8.9 (See SEQ ID NO: 45 in | AAV CSp-8.9 (See SEQ ID NO: 95 in |
| WO2016065001) | WO2016065001) |
| AAV CSp-9 842 (See SEQ ID NO: 54 in | AAV CSp-9 (See SEQ ID NO: 128 in |
| U.S. Pat. No. 8,734,809) | U.S. Pat. No. 8,734,809) |
| AAV.hu.48R3 (See SEQ ID NO: 183 in | AAV.VR-355 (See SEQ ID NO: 181 in |
| U.S. Pat. No. 8,734,809) | U.S. Pat. No. 8,734,809) |
| AAV3B (See SEQ ID NO: 48 in WO2016065001) | AAV3B (See SEQ ID NO: 98 in |
| | WO2016065001) |
| AAV4 (See SEQ ID NO: 49 in WO2016065001) | AAV4 (See SEQ ID NO: 99 in |
| | WO2016065001) |
| AAV5 (See SEQ ID NO: 50 in WO2016065001) | AAV5 (See SEQ ID NO: 100 in |
| | WO2016065001) |
| AAVF1/HSC1 (See SEQ ID NO: 20 in | AAVF1/HSC1 (See SEQ ID NO: 2 in |
| WO2016049230) | WO2016049230) |
| AAVF11/HSC11 (See SEQ ID NO: 26 in | AAVF11/HSC11 (See SEQ ID NO: 4 in |
| WO2016049230) | WO2016049230) |
| AAVF12/HSC12 (See SEQ ID NO: 30 in | AAVF12/HSC12 (See SEQ ID NO: 12 in |
| WO2016049230) | WO2016049230) |
| AAVF13/HSC13 (See SEQ ID NO: 31 in | AAVF13/HSC13 (See SEQ ID NO: 14 in |
| WO2016049230) | WO2016049230) |
| AAVF14/HSC14 (See SEQ ID NO: 32 in | AAVF14/HSC14 (See SEQ ID NO: 15 in |
| WO2016049230) | WO2016049230) |
| AAVF15/HSC15 (See SEQ ID NO: 33 in | AAVF15/HSC15 (See SEQ ID NO: 16 in |
| WO2016049230) | WO2016049230) |
| AAVF16/HSC16 (See SEQ ID NO: 34 in | AAVF16/HSC16 (See SEQ ID NO: 17 in |
| WO2016049230) | WO2016049230) |
| AAVF17/HSC17 (See SEQ ID NO: 35 in | AAVF17/HSC17 (See SEQ ID NO: 13 in |
| WO2016049230) | WO2016049230) |
| AAVF2/HSC2 (See SEQ ID NO: 21 in | AAVF2/HSC2 (See SEQ ID NO: 3 in |
| WO2016049230) | WO2016049230) |
| AAVF3/HSC3 (See SEQ ID NO: 22 in | AAVF3/HSC3 (See SEQ ID NO: 5 in |
| WO2016049230) | WO2016049230) |
| AAVF4/HSC4 (See SEQ ID NO: 23 in | AAVF4/HSC4 (See SEQ ID NO: 6 in |
| WO2016049230) | WO2016049230) |
| AAVF5/HSC5 (See SEQ ID NO: 25 in | AAVF5/HSC5 (See SEQ ID NO: 11 in |
| WO2016049230) | WO2016049230) |
| AAVF6/HSC6 (See SEQ ID NO: 24 in | AAVF6/HSC6 (See SEQ ID NO: 7 in |
| WO2016049230) | WO2016049230) |
| AAVF7/HSC7 (See SEQ ID NO: 27 in | AAVF7/HSC7 (See SEQ ID NO: 8 in |
| WO2016049230) | WO2016049230) |
| AAVF8/HSC8 (See SEQ ID NO: 28 in | AAVF8/HSC8 (See SEQ ID NO: 9 in |
| WO2016049230) | WO2016049230) |
| AAVF9/HSC9 (See SEQ ID NO: 10 in | AAVF9/HSC9 882 (see SEQ ID NO: 29 in |
| WO2016049230) | WO2016049230) |

In one embodiment, the AAV vector (also referred to as a rAAV virion) as disclosed herein comprises a capsid protein from any of those disclosed in WO2019/241324, which is specifically incorporated herein in its entirety by reference. In some embodiments of the invention described herein, the rAAV vector comprises a liver specific capsid, e.g., a liver specific capsid selected from XL32 and XL32.1, as disclosed in WO2019/241324, which is incorporated herein in its entirety by reference. In some embodiments, the rAAV vector is a AAVXL32 or AAVXL32.1 as disclosed in WO2019/241324, which is incorporated herein in its entirety by reference.

Exemplary chimeric or variant capsid proteins that can be used as the AAV capsid in the rAAV vector described herein can be selected from Table 2 from U.S. provisional application 62,937,556, filed on Nov. 19, 2019, which is specifically incorporated herein in its reference, or can be used with any combination with wild type capsid proteins and/or other chimeric or variant capsid proteins now known or later identified and each is incorporated herein. In some embodiments, the rAAV vector encompassed for use is a chimeric vector, e.g., as disclosed in 9,012,224 and U.S. Pat. No. 7,892,809, which are incorporated herein in their entirety by reference.

In some embodiments, the rAAV vector is a haploid rAAV vector, as disclosed in US application US2018/0371496 and PCT/US18/22725, or polyploid rAAV vector, e.g., as disclosed in PCT/US2018/044632 filed on Jul. 31, 2018 and in U.S. application Ser. No. 16/151,110, each of which are incorporated herein in their entirety by reference. In some embodiments, the rAAV vector is a rAAV3 vector, as disclosed in 9,012,224 and WO 2017/106236 which are incorporated herein in their entirety by reference.

In a particular embodiment, the rAAV is a AAVXL32 or AAVXL32.1 AAV vector as disclosed in WO2019/241324, which is incorporated herein in its entirety by reference. In some embodiments, the rAAV vector comprises a capsid disclosed in WO2019241324A1, or International Patent application PCT/US2019/036676, which are incorporated herein in their entirety by reference. In some embodiments, the AAV vector is a AAV8 vector or a rational haploid comprising an AAV8 capsid protein. In some embodiments, the recombinant AAV vector is a chimeric AAV vector, haploid AAV vector, a hybrid AAV vector or polyploid AAV vector. In some embodiments, the recombinant AAV vector is a rational haploid vector, a mosaic AAV vector, a chemically modified AAV vector, or a AAV vector from any AAV serotypes, for example, from any AAV serotype disclosed in Table 1 as disclosed in International Applications WO2020/102645, and WO2020/102667, each of which are incorporated herein in their entirety.

In an embodiment, an rAAV vector useful in the treatment of Pompe Disease as disclosed herein is an AAV3b capsid. AAV3b capsids encompassed for use are described in 2017/106236, and 9,012,224 and 7,892,809, and International application PCT/US19/61653, filed Nov. 15, 2019, and International Applications WO2020/102645, and WO2020/102667, each of which are incorporated herein in their entirety. In addition, AAV3b capsids of the AAV vector for use according to the methods as disclosed herein are disclosed in International Patent Applications WO 2020/102645 and WO2021102107, which are incorporated herien in its entirity by reference herein.

In some embodiments, the AAV3b capsid comprises SEQ ID NO: 44 as disclosed in International Patent Applications WO 2020/102645 and WO2021102107. In an embodiment, the AAV capsid used in the treatment of Pompe Disease can be a modified AAV capsid that is derived in whole or in part from the AAV capsid set forth in SEQ ID NO: 44. In some embodiments, the amino acids from an AAV3b capsid as set forth in SEQ ID NO: 44 can be, or are substituted with amino acids from another capsid of a different AAV serotype, wherein the substituted and/or inserted amino acids can be from any AAV serotype, and can include either naturally occurring or partially or completely synthetic amino acids.

In another embodiment, an AAV capsid used in the treatment of Pompe Disease is an AAV3b265D capsid. In this particular embodiment, an AAV3b265D capsid comprises a modification in the amino acid sequence of the two-fold axis loop of an AAV3b capsid via replacement of amino acid G265 of the AAV3b capsid with D265. In some embodiments, an AAV3b265D capsid comprises SEQ ID NO: 46. However, the modified virus capsids of the invention are not limited to AAV capsids set forth in SEQ ID NO: 46 as set forth in International Patent Applications WO 2020/102645 and WO2021102107. In some embodiments, the amino acids from AAV3b265D as set forth in SEQ ID NO. 46 can be, or are substituted with amino acids from a capsid from an AAV of a different serotype, wherein the substituted and/or inserted amino acids can be from any AAV serotype, and can include either naturally occurring or partially or completely synthetic amino acids.

In another embodiment an rAAV vector useful in the treatment of Pompe Disease as disclosed herein is an AAV3b265D549A capsid. In this particular embodiment, an AAV3b265D549A capsid comprises a modification in the amino acid sequence of the two-fold axis loop of an AAV3b capsid via replacement of amino acid G265 of the AAV3b capsid with D265 and replacement of amino acid T549 of the AAV3b capsid with A549. In some embodiments, an AAV3b265D549A capsid comprises SEQ ID NO: 50 as disclosed herein International Patent Applications WO 2020/102645 and WO2021102107. However, the modified virus capsids of the invention are not limited to AAV capsids set forth in SEQ ID NO: 50. In some embodiments, the amino acids from AAV3b265D549A as set forth in SEQ ID NO: 50 can be, or are substituted with amino acids from a capsid from an AAV of a different serotype, wherein the substituted and/or inserted amino acids can be from any AAV serotype, and can include either naturally occurring or partially or completely synthetic amino acids. In some embodiments, the amino acids from AAV3bSASTG (i.e., a AAV3b capsid comprising Q263A/T265 mutations) can be, or are substituted with amino acids from a capsid from an AAV of a different serotype, wherein the substituted and/or inserted amino acids can be from any AAV serotype, and can include either naturally occurring or partially or completely synthetic amino acids.

In another embodiment, an rAAV vector useful in the treatment of Pompe Disease as disclosed herein is an AAV3b549A capsid. In this particular embodiment, an AAV3b549A capsid comprises a modification in the amino acid sequence of the two-fold axis loop of an AAV3b capsid via replacement of amino acid T549 of the AAV3b capsid with A549. In some embodiments, an AAV3b549A capsid comprises SEQ ID NO: 52 as disclosed herein International Patent Applications WO 2020/102645 and WO2021102107. However, the modified virus capsids of the invention are not limited to AAV capsids set forth in SEQ ID NO: 52. In some embodiments, the amino acids from AAV3b549A as set forth in SEQ ID NO: 52 can be, or are substituted with amino acids from a capsid from an AAV of a different serotype, wherein the substituted and/or inserted amino acids can be from any AAV serotype, and can include either naturally occurring or partially or completely synthetic amino acids.

In another embodiment, an rAAV vector useful in the treatment of Pompe Disease as disclosed herein is an AAV3bQ263Y capsid. In this particular embodiment, an AAV3bQ263Y capsid comprises a modification in the amino acid sequence of the two-fold axis loop of an AAV3b capsid via replacement of amino acid Q263 of the AAV3b capsid with Y263. In some embodiments, an AAV3b549A capsid comprises SEQ ID NO: 54 as disclosed herein International Patent Applications WO 2020/102645 and WO2021102107. However, the modified virus capsids of the invention are not limited to AAV capsids set forth in SEQ ID NO: 54. In some embodiments, the amino acids from AAV3bQ263Y as set forth in SEQ ID NO: 54 can be, or are substituted with amino acids from a capsid from an AAV of a different serotype, wherein the substituted and/or inserted amino acids can be from any AAV serotype, and can include either naturally occurring or partially or completely synthetic amino acids.

In another embodiment, an rAAV vector useful in the treatment of Pompe Disease as disclosed herein is AAV3bSASTG serotype or comprises a AAV3bSASTG capsid. In this particular embodiment, an AAV3bSASTG capsid comprises a modification in the amino acid sequence to comprise a SASTG mutation, in particular, the AAV3b capsid was modified to resemble AAV2 Q263A/T265 subvariant by introducing these modifications at similar positions in the AAV3b capsid (as disclosed in Messina E L, et al., Adeno-associated viral vectors based on serotype 3b use components of the fibroblast growth factor receptor signaling complex for efficient transduction. Hum. Gene Ther. 2012 October: 23(10):1031-4, Piacentino III, Valentino, et al. “X-linked inhibitor of apoptosis protein-mediated attenuation of apoptosis, using a novel cardiac-enhanced adeno-associated viral vector.” Human gene therapy 23.6 (2012): 635-646. which are both incorporated herein in their entirety by reference). Accordingly, in some embodiments, an rAAV vector useful in the treatment of Pompe Disease as disclosed herein is AAV3bSASTG serotype or comprises a AAV3bSASTG capsid comprising a AAV3b Q263A/T265 capsid. In some embodiments, the amino acids from AAV3bSASTG can be, or are substituted with amino acids from a capsid from an AAV of a different serotype, wherein the substituted and/or inserted amino acids can be from any AAV serotype, and can include either naturally occurring or partially or completely synthetic amino acids.

One can target desired tissues using the appropriate capsids. For example, the central nervous system using AAV9 or a rhesus capsid or a rational haploid using at least one of a AAV9 or Rhesus viral protein. One can target the muscle using myo AAV, see, e.g., WO2019/2071323 and WO2022/020616, which are incorporated herein in their entirity by reference.

In order to facilitate their introduction into a cell, an rAAV vector genome useful in the invention are recombinant nucleic acid constructs that include (1) a heterologous sequence to be expressed (in one embodiment, a polynucleotide encoding a GAA polypeptide) and (2) viral sequence elements that facilitate integration and expression of the heterologous genes. The viral sequence elements may include those sequences of an AAV vector genome that are required in cis for replication and packaging (e.g., functional ITRs) of the DNA into an AAV capsid. In an embodiment, the heterologous gene encodes GAA, which is useful for correcting a GAA-deficiency in a patient suffering from Pompe Disease. In an embodiment, such an rAAV vector genome may also contain marker or reporter genes. In an embodiment, an rAAV vector genome can have one or more of the AAV3b wild-type (WT) cis genes replaced or deleted in whole or in part, but retain functional flanking ITR sequences.

III. Optimized rAAV Vector Genome

In some embodiments of the methods and compositions as disclosed herein, an optimized rAAV vector genome is created from any of the elements disclosed herein and in any combination, including nucleic acid sequences encoding a promoter, an ITR, a poly-A tail, elements capable of increasing or decreasing expression of a heterologous gene, and in one embodiment, a nucleic acid sequence that is codon optimized for expression of GAA protein in vivo (i.e., wildtype GAA or codon optimized GAA) and optionally, one or more element to reduce immunogenicity. Such an optimized rAAV vector genome can be used with any AAV capsid that has tropism for the tissue and cells in which the rAAV vector genome is to be transduced and expressed.

In some embodiments, rAAV genome lacks the AAV P5 promoter or, a fragment thereof, which is normally located upstream of the liver-specific promoter as disclosed herein. Normally, the P5 promoter controls expression of the AAV rep/cap proteins during AAV replication. In some embodiments, this P5 promoter fragment is present in the rAAV vector as disclosed herein which contains predicted transcription factor binding sites, e.g., cyclic AMP-responsive element-binding protein 3 (CREB3), which can be activated by endoplasmic reticulum (ER)/Golgi stress (Sampieri 2019), activating transcription factor 2 (ATF2), which is also involved in stress response (Watson 2017), Nuclear Receptor Subfamily 1 Group I Member 2 (NR1I2) (also known as Pregnane X receptor [PXR]) is known to be enriched in liver, and is activated by pregnane steroids, rifampin and other molecules including dexamethasone (NR1I2 HGNC) (Xing 2020). Accordingly, in some embodiments, a fragment of the AAV P5 promoter in the rAAV genome is removed without affecting the intended performance of the GAA cassette. In some embodiments, the rAAV vector also comprises a RNA polymerase II termination sequence located between the polyA signal and the 3′ ITR. An exemplary terminal sequence is SEQ ID NO: 45, or SEQ ID NO:465, the later of which introduces two termination codons and one restriction site (e.g., XhoI) replaces TAG, and is located immediately downstream of the last coding amino acids of hGAA, and immediately located upstream of the 3′ UTR.

### IV. Subjects Amenable to Treatment

### A. Pompe Disease

The recombinant AAV expressing GAA protein as disclosed herein can be used in methods to treat Pompe disease. Pompe disease is a rare genetic disorder caused by a deficiency in the enzyme acid alpha-glucosidase (GAA), which is needed to break down glycogen, a stored form of sugar used for energy. Pompe disease is also known as glycogen storage disease type II, GSD II, type II glycogen storage disease, glycogenosis type II, acid maltase deficiency, alpha-1,4-glucosidase deficiency, cardiomegalia glycogenic diffusa, and cardiac form of generalized glycogenosis. The build-up of glycogen causes progressive muscle weakness (myopathy) throughout the body and affects various body tissues, particularly in the heart, skeletal muscles, liver, respiratory and nervous system.

Glycogen storage disease type II, also referred to as Pompe disease, is a rare disorder of metabolism inherited in an autosomal recessive manner, caused by deficiency of the lysosomal enzyme acid α-glucosidase (GAA). This disorder leads to the accumulation of lysosomal glycogen and destruction of skeletal, smooth and cardiac muscle. Pompe disease ranges in severity from a severe, infantile-onset myopathy accompanied by severe hypotonia and hypertrophic cardiomyopathy (infantile-onset Pompe disease [IOPD]) to a late-onset myopathy (LOPD). The defect in GAA can vary from complete to partial deficiency of GAA, which correlates with clinical severity. LOPD presents with proximal leg weakness and in some cases respiratory insufficiency without significant cardiac involvement and may progress to fatal respiratory failure.

Early onset (or the infantile form, IOPD) is the result of complete or near complete deficiency of GAA. Symptoms begin in the first months of life and progress rapidly, with feeding problems, poor weight gain, muscle weakness, floppiness, and head lag. Respiratory difficulties are often complicated by lung infections. The heart is grossly enlarged. Many infants with Pompe disease also have enlarged tongues. If untreated with Lumizyme, most babies die from cardiac or respiratory complications before their first birthday.

Late onset (or juvenile/adult, LOPD) Pompe disease is the result of a partial deficiency of GAA. The onset can be as early as the first decade of childhood or as late as the sixth decade of adulthood and is therefore characterized as slowly progressive. The primary symptom is proximal muscle weakness progressing to respiratory weakness and death from respiratory failure after a course lasting several years. The heart is usually not involved.

The presenting clinical manifestations of Pompe disease can vary widely depending on the age of disease onset and residual GAA activity. Residual GAA activity correlates with both the amount and tissue distribution of glycogen accumulation as well as the severity of the disease. Infantile-onset Pompe disease (less than 1% of normal GAA activity) is the most severe form and is characterized by hypotonia, generalized muscle weakness, and hypertrophic cardiomyopathy, and massive glycogen accumulation in cardiac and other muscle tissues. Death usually occurs within one year of birth due to cardiorespiratory failure. Juvenile-onset (1-10% of normal GAA activity) and adult-onset (10-40% of normal GAA activity) Pompe disease are more clinically heterogeneous, with greater variation in age of onset, clinical presentation, and disease progression. Juvenile- and adult-onset Pompe disease are generally characterized by lack of severe cardiac involvement, later age of onset, and slower disease progression, but eventual respiratory or limb muscle involvement results in significant morbidity and mortality. While life expectancy can vary, death generally occurs due to respiratory failure.

In any embodiment of the methods as disclosed herein, a GAA enzyme suitable for treating Pompe disease includes a wild-type human GAA, or a fragment or sequence variant thereof which retains the ability to cleave al-4 linkages in linear oligosaccharides. In some embodiments of the methods and compositions as disclosed herein, the GAA protein encoded by a GAA nucleic acid sequence, e.g., SEQ ID NO: 1-18 as disclosed herein, or a N-terminal truncation thereof as disclosed herein in Table 1. In some embodiments of the methods and compositions as disclosed herein, the GAA protein is encoded by a codon optimized GAA nucleic acid sequence, for example, for any one or more of: (1) enhanced expression in vivo, (2) to reduce CpG islands or (3) reduce the innate immune response. In some embodiments of the methods and compositions as disclosed herein, the GAA protein is encoded by a codon optimized GAA nucleic sequence, for example, any nucleic acid sequence selected from any of: SEQ ID NO: 1-18, or a nucleic acid sequence having at least 60%, or 70%, or 80%, 85% or 90% or 95%, or 98%, or 99% sequence identity to SEQ ID NOS: 1-18, which encode a GAA polypeptide, where amino acid at position 199 is R (199R); amino acid at position 233 is H (233H), and amino acid at position 780 is I (780I), as compared to the wild type GAA protein.

In some embodiments of the methods and compositions as disclosed herein, a rAAV vector as described herein transduces the liver of a subject and secretes the hGAA polypeptide into the blood, which perfuses patient tissues where the hGAA polypeptide, is taken up by cells and transported to the lysosome, where the GAA enzyme acts to eliminate material that has accumulated in the lysosomes due to the enzyme deficiency. For lysosomal enzyme replacement therapy to be effective, the therapeutic enzyme must be delivered to lysosomes in the appropriate cells in tissues where the storage defect is manifest.

In some embodiments, upon administration, the AAV vector selectively expresses and secretes GAA from transduced hepatocytes. The primary mechanism of action of a AAV vector expressing hGAA polypeptide as disclosed herein is to secrete continuous low levels of endogenous GAA from the liver into the systemic circulation in order to provide therapeutic exposure levels of GAA to tissue (e.g., the muscle, but not exclusively the muscle), resulting in glycogen removal and restoration of cellular architecture and function.

B. Increasing GAA Activity in A Subject with Pompe Disease

In any embodiment of the methods as disclosed herein, administration of a AAV vector expressing GAA is administration to a muscle, and can be by any suitable method including intravenous administration, intra-arterial administration, and/or intra-peritoneal administration. Exemplary modes of administration include oral, rectal, transmucosal, intranasal, inhalation (e.g., via an aerosol), buccal (e.g., sublingual), vaginal, intrathecal, intraocular, transdermal, in utero (or in ovo), parenteral (e.g., intravenous, subcutaneous, intradermal, intramuscular [including administration to skeletal, diaphragm and/or cardiac muscle], intradermal, intrapleural, intracerebral, and intraarticular), topical (e.g., to both skin and mucosal surfaces, including airway surfaces, and transdermal administration), intralymphatic, and the like, as well as direct tissue or organ injection (e.g., to liver, skeletal muscle, cardiac muscle, diaphragm muscle or brain). The most suitable route in any given case will depend on the nature and severity of the condition being treated and/or prevented and on the nature of the particular vector that is being used.

In any embodiment of the methods as disclosed herein, administration of a AAV vector expressing GAA as disclosed herein is to skeletal muscle according to the present invention, and includes but is not limited to administration to skeletal muscle in the limbs (e.g., upper arm, lower arm, upper leg, and/or lower leg), back, neck, head (e.g., tongue), thorax, abdomen, pelvis/perineum, and/or digits. Suitable skeletal muscles that can be injected are disclosed in International Application WO2021102107, which is incorporated herein its entirety by reference.

In any embodiment of the methods as disclosed herein, the rAAV vectors and/or rAAV genome are administered to the skeletal muscle, liver, diaphragm, costal, and/or cardiac muscle cells of a subject. For example, a conventional syringe and needle can be used to inject a rAAV virion suspension into an animal. Parenteral administration of a the rAAV vectors and/or rAAV genome, by injection can be performed, for example, by bolus injection or continuous infusion. Formulations for injection may be presented in unit dosage form, for example, in ampoules or in multi-dose containers, with an added preservative. The compositions may take such forms as suspensions, solutions or emulsions in oily or aqueous vehicles, and may contain agents for a pharmaceutical formulation, such as suspending, stabilizing and/or dispersing agents. Alternatively, the rAAV vectors and/or rAAV genome as disclosed herein can be in powder form (e.g., lyophilized) for constitution with a suitable vehicle, for example, sterile pyrogen-free water, before use.

In particular embodiments, more than one administration (e.g., two, three, four, five, six, seven, eight, nine, 10, etc., or more administrations) may be employed to achieve the desired level of GAA expression over a period of various intervals, e.g., hourly, daily, weekly, monthly, yearly, etc. Dosing can be single dosage or cumulative (serial dosing), and can be readily determined by one skilled in the art. As disclosed herein, it is envisioned that treatment of Pompe Disease according to the methods as disclosed herein comprises a one-time administration of an effective dose of a pharmaceutical composition comprising a AAV vector encoding a GAA polypeptide.

However, in alternative embodiments, treatment of a subject with Pompe disease may comprise multiple administrations of a pharmaceutical composition comprising a AAV vector encoding a GAA polypeptide when the subject is not administered long-term ERT, where the multiple administrations can be carried out over a range of time periods, such as, e.g., once yearly, or every 6-months, or about every 2-years, or about every 3-years, or about every 4 years, or about every 5-years or longer than 5-year intervals. The timing of administration can vary from individual to individual, depending upon such factors as the severity of an individual's symptoms. For example, in some embodiments, an effective dose of a AAV vector encoding a GAA polypeptide as disclosed herein can be administered to an individual once every year, or once every two years, or every six months for an indefinite period of time, or until the individual no longer requires therapy. A person of ordinary skill in the art will recognize that the condition of the individual can be monitored throughout the course of treatment and that the effective amount of a AAV vector encoding a GAA polypeptide as disclosed herein that is administered can be adjusted accordingly.

Injectables comprising a AAV vector encoding a GAA polypeptide as disclosed herein can be prepared in conventional forms, either as liquid solutions or suspensions, solid forms suitable for solution or suspension in liquid prior to injection, or as emulsions. Alternatively, one may administer a AAV vector encoding a GAA polypeptide as disclosed herein in a local rather than systemic manner, for example, in a depot or sustained-release formulation. Further, the virus vector and/or virus capsid can be delivered adhered to a surgically implantable matrix (e.g., as described in U.S. Patent Publication No. US-2004-0013645-A1). In some embodiments, a AAV vector encoding a GAA polypeptide as disclosed herein can be administered to the lungs of a subject by any suitable means, optionally by administering an aerosol suspension of respirable particles comprised of the virus vectors and/or virus capsids, which the subject inhales. The respirable particles can be liquid or solid. Aerosols of liquid particles comprising the virus vectors and/or virus capsids may be produced by any suitable means, such as with a pressure-driven aerosol nebulizer or an ultrasonic nebulizer, as is known to those of skill in the art. See, e.g., U.S. Pat. No. 4,501,729. Aerosols of solid particles comprising the virus vectors and/or capsids may likewise be produced with any solid particulate medicament aerosol generator, by techniques known in the pharmaceutical art.

In some embodiments, a AAV vector encoding a GAA polypeptide as disclosed herein can be formulated in a solvent, emulsion or other diluent in an amount sufficient to dissolve an rAAV vector disclosed herein. In other aspects of this embodiment, the rAAV vectors and/or rAAV genome encoding GAA polypeptide as disclosed herein can herein may be formulated in a solvent, emulsion or a diluent in an amount of, e.g., less than about 90% (v/v), less than about 80% (v/v), less than about 70% (v/v), less than about 65% (v/v), less than about 60% (v/v), less than about 55% (v/v), less than about 50% (v/v), less than about 45% (v/v), less than about 40% (v/v), less than about 35% (v/v), less than about 30% (v/v), less than about 25% (v/v), less than about 20% (v/v), less than about 15% (v/v), less than about 10% (v/v), less than about 5% (v/v), or less than about 1% (v/v). In other aspects, the rAAV vectors and/or rAAV genome encoding a GAA polypeptide as disclosed herein can disclosed herein may comprise a solvent, emulsion or other diluent in an amount in a range of, e.g., about 1% (v/v) to 90% (v/v), about 1% (v/v) to 70% (v/v), about 1% (v/v) to 60% (v/v), about 1% (v/v) to 50% (v/v), about 1% (v/v) to 40% (v/v), about 1% (v/v) to 30% (v/v), about 1% (v/v) to 20% (v/v), about 1% (v/v) to 10% (v/v), about 2% (v/v) to 50% (v/v), about 2% (v/v) to 40% (v/v), about 2% (v/v) to 30% (v/v), about 2% (v/v) to 20% (v/v), about 2% (v/v) to 10% (v/v), about 4% (v/v) to 50% (v/v), about 4% (v/v) to 40% (v/v), about 4% (v/v) to 30% (v/v), about 4% (v/v) to 20% (v/v), about 4% (v/v) to 10% (v/v), about 6% (v/v) to 50% (v/v), about 6% (v/v) to 40% (v/v), about 6% (v/v) to 30% (v/v), about 6% (v/v) to 20% (v/v), about 6% (v/v) to 10% (v/v), about 8% (v/v) to 50% (v/v), about 8% (v/v) to 40% (v/v), about 8% (v/v) to 30% (v/v), about 8% (v/v) to 20% (v/v), about 8% (v/v) to 15% (v/v), or about 8% (v/v) to 12% (v/v).

In any embodiment of the methods as disclosed herein, a AAV vector encoding a GAA polypeptide can be an AAV of any serotype, including but not limited to encapsulated by any AAV8 capsid, or any AAV3b capsid selected from: AAV3b capsid (SEQ ID NO: 452); AAV3b265D capsid (SEQ ID NO: 454), AAV3b ST (S663V+T492V) capsid (SEQ ID NO: 456), AAV3b265D549A capsid (SEQ ID NO: 458); AAV3b549A capsid (SEQ ID NO: 460); AAV3bQ263Y capsid (SEQ ID NO: 462) or AAV3bSASTG capsid (i.e., a AAV3b capsid comprising Q263A/T265 mutations).

To facilitate delivery of a AAV vector encoding a GAA polypeptide as disclosed herein, it can be mixed with a carrier or excipient. Carriers and excipients that might be used include saline (especially sterilized, pyrogen-free saline) saline buffers (for example, citrate buffer, phosphate buffer, acetate buffer, and bicarbonate buffer), amino acids, urea, alcohols, ascorbic acid, phospholipids, proteins (for example, serum albumin), EDTA, sodium chloride, liposomes, mannitol, sorbitol, and glycerol. USP grade carriers and excipients are particularly useful for delivery of virions to human subjects.

In addition to the formulations described previously, a AAV vector encoding a GAA polypeptide as disclosed herein can also be formulated as a depot preparation. Such long acting formulations may be administered by implantation (for example subcutaneously or intramuscularly) or by IM injection. Thus, for example, a rAAV vector and/or rAAV genome as disclosed herein may be formulated with suitable polymeric or hydrophobic materials (for example as an emulsion in an acceptable oil) or ion exchange resins, or as sparingly soluble derivatives.

In any embodiment of the methods as disclosed herein, the method is directed to treating Pompe Disease that results from a deficiency of GAA in a subject, wherein a AAV vector encoding a GAA polypeptide as disclosed herein is administered to a patient suffering from Pompe Disease, and following administration, GAA is secreted from cells in the liver and there is uptake of the secreted GAA by cells in skeletal muscle tissue, cardiac muscle tissue, diaphragm muscle tissue or a combination thereof, wherein uptake of the secreted GAA results in a reduction in lysosomal glycogen stores in the tissue(s), including but not limited to muscle. In some embodiments, a AAV vector encoding a GAA polypeptide as disclosed herein is encapsulated in a capsid, e.g., encapsulated by any AAV3b capsid selected from: AAV3b capsid (SEQ ID NO: 452); AAV3b265D capsid (SEQ ID NO: 454), AAV3b ST (S663V+T492V) capsid (SEQ ID NO: 456), AAV3b265D549A capsid (SEQ ID NO: 458); AAV3b549A capsid (SEQ ID NO: 460); AAV3bQ263Y capsid (SEQ ID NO: 462) or AAV3bSASTG capsid (i.e., a AAV3b capsid comprising Q263A/T265 mutations).

In a particular embodiment, at least about 1.6×10¹² to about 4.0×10¹² vg/kg will be administered per dose in a pharmaceutically acceptable carrier. In a further embodiment, dosages of the virus vector and/or capsid to be administered to a subject depend upon the mode of administration, the severity and type of Pompe disease (i.e., LOPD or IOPD) to be treated and/or prevented, the individual subject's condition, age and gender, and the particular virus vector or capsid, the nucleic acid encoding GAA polypeptide to be delivered, and the like, and can be determined in a routine manner.

Exemplary doses for achieving therapeutic effects are titers of at least about between 1.0E9 vg/kg and 5.0E13 vg/kg, e.g., 1.0E9 vg/kg and 5.0E12 vg/kg; 5.0E9 vg/kg and 5.0E12 vg/kg; 5.0E9 vg/kg and 1.0E12 vg/kg; 5.0E9 vg/kg and 5.0E11 vg/kg; 5.0E9 vg/kg and 5.0E10 vg/kg; and 1.0E9 vg/kg and 1.0E10 vg/kg.1.5×10¹¹ vg/kg, or at least about 1.5×10¹² vg/kg, or at least about 4.0×10¹² vg/kg. It is encompassed that the dose for achieving therapeutic effects as disclosed herein may also be determined by the strength of the liver specific promoter (LSP) operatively linked to the nucleic acid encoding the GAA polypeptide, as well as specific signal sequence, and ability of the cell to cleave the signal sequence when secreted from the cell. In contrast, the dose of the AAV encoding the GAA polypeptide as disclosed herein can be lower than about 1.6×10¹² when the liver specific promoter is stronger than the LPS (SEQ ID NO: 97) used in a AAV8-LSPhGAA vector, however, the dose of AAV should be titrated and determined based on the level of GAA expressed in the cell, as determined by transduction efficiency of the AAV capsid and the LSP, and the ability of the cell to secrete the expressed GAA polypeptide in order to avoid GAA accumulation in the transfected cell and any associated cell toxicity.

In another aspect, disclosed herein is a method of treating Pompe Disease by administering a nucleic acid encoding a GAA to a cell, comprising contacting the cell with a rAAV vector and/or rAAV genome as disclosed herein, under conditions for the nucleic acid to be introduced into the cell and expressed to produce GAA. In some embodiments, the cell is a cell in vivo. In some embodiments, the cell is a mammalian cell in vivo.

### C. Increasing Motoneuron Function In A Mammal

In any embodiment of the methods as disclosed herein, a AAV vector encoding a GAA polypeptide as disclosed herein is useful in methods to increase phrenic nerve activity in a mammal having Pompe disease and/or insufficient GAA levels. For example, a AAV vector encoding a GAA polypeptide as disclosed herein, e.g., a rAAV vector and/or rAAV genome encapsulated in a capsid, e.g., encapsulated by AAV8 or any AAV3b capsid selected from: AAV3b capsid (SEQ ID NO: 452); AAV3b265D capsid (SEQ ID NO: 454), AAV3b ST (S663V+T492V) capsid (SEQ ID NO: 456), AAV3b265D549A capsid (SEQ ID NO: 458); AAV3b549A capsid (SEQ ID NO: 460); AAV3bQ263Y capsid (SEQ ID NO: 462) or AAV3bSASTG capsid, can be administered to the central nervous system (e.g., neurons). In another embodiment, retrograde transport a AAV vector encoding a GAA polypeptide as disclosed herein from the diaphragm (or other muscle) to the phrenic nerve or other motor neurons can result in biochemical and physiological correction of Pompe disease. These same principles could be applied to other neurodegenerative disease.

In an embodiment, a rAAV capsid of the rAAV virion used to treat Pompe Disease is any of those listed in Table 1 as disclosed in International Applications WO2020/102645, and WO2020/102667, each of which are incorporated herein in their entirety, and includes any of AAV8 or AAV3, or AAV3b (including but not limited to AAV3b serotypes AAV3b265D, AAV3b265D549A, AAV3b549A, AAV3bQ263Y, AAV3bSASTG (i.e., a AAV3b capsid comprising Q263A/T265 mutations) serotypes) is capable of reducing any one or more of the symptoms of (i) the feeling of weakness in a patient's lower extremities, including, the legs, trunk and/or arms, (ii) a shortness of breath, a hard time exercising, lung infections, a big curve in the spine, trouble breathing while sleeping, an enlarged liver, an enlarged tongue and/or a stiff joint, (iii) in a patient suffering from Pompe Disease by, e.g., at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90% or at least 95% as compared to a patient not receiving the same treatment. In other aspects of this embodiment, an AAV GAA of any serotype is capable of reducing any one or more of the systems of (i) the feeling of weakness in a patient's lower extremities, including, the legs, trunk and/or arms, ii) a shortness of breath, a hard time exercising, lung infections, a big curve in the spine, trouble breathing while sleeping, an enlarged liver, an enlarged tongue and/or a stiff joint, (iii) in a patient suffering from Pompe Disease by, e.g., about 10% to about 100%, about 20% to about 100%, about 30% to about 100%, about 40% to about 100%, about 50% to about 100%, about 60% to about 100%, about 70% to about 100%, about 80% to about 100%, about 10% to about 90%, about 20% to about 90%, about 30% to about 90%, about 40% to about 90%, about 50% to about 90%, about 60% to about 90%, about 70% to about 90%, about 10% to about 80%, about 20% to about 80%, about 30% to about 80%, about 40% to about 80%, about 50% to about 80%, or about 60% to about 80%, about 10% to about 70%, about 20% to about 70%, about 30% to about 70%, about 40% to about 70%, or about 50% to about 70% as compared to a patient not receiving the same treatment.

In any embodiment of the methods and compositions as disclosed herein, at least one symptom associated with Pompe Disease, or at least one adverse side effect associated with Pompe Disease are reduced by at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95%, and the severity of at least one symptom associated with Pompe Disease, or at least one adverse side effect is reduced by at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95%. In another embodiment, at least one symptom associated with Pompe Disease, or at least one adverse side effect associated with Pompe Disease is reduced by about 10% to about 100%, about 20% to about 100%, about 30% to about 100%, about 40% to about 100%, about 50% to about 100%, about 60% to about 100%, about 70% to about 100%, about 80% to about 100%, about 10% to about 90%, about 20% to about 90%, about 30% to about 90%, about 40% to about 90%, about 50% to about 90%, about 60% to about 90%, about 70% to about 90%, about 10% to about 80%, about 20% to about 80%, about 30% to about 80%, about 40% to about 80%, about 50% to about 80%, or about 60% to about 80%, about 10% to about 70%, about 20% to about 70%, about 30% to about 70%, about 40% to about 70%, or about 50% to about 70%.

### V. Methods of Administration

Without wishing to be bound by theory, the only current treatment for Pompe disease is long-term administration of recombinant human GAA (rhGAA) ERT, which is typically administered on an every-other week regimen. Herein, the inventors have demonstrated that a subject with Pompe disease can take breaks from the normal ERT regimen for extended period of time (e.g., extended periods of ERT cessation) without a clinical set back if the subject is administered a specific dose of AAV vector expressing a GAA polypeptide as disclosed herein. In some embodiments, withdrawal of the administration of long-term ERT begins at about the time of administration of the AAV vector to the subject (e.g., the day before, the day of, or the day after), or in some embodiments, withdrawal of the administration of long-term ERT can occur at about 26 weeks, or anywhere within about 24 to about 26 weeks after administration of the AAV vector.

In some embodiments, a subject administered a AAV vector expressing GAA as disclosed herein, can, after an initial period of withdrawal of the administration of long-term ERT for an extended period of time, be administered complementary ERT, where the complementary ERT is administered after about 6-months, or about 1 year, or longer than a year of cessation of the long-term ERT. Stated differently and without wishing to be bound by theory, the technology disclosed herein relates to a method whereby a subject with Pompe disease who is administered a AAV vector expressing GAA as disclosed herein, can have breaks or “holidays” from the normal long-term ERT administration. That is, according to the methods as disclosed herein, a subject administered an AAV vector expressing GAA as disclosed herein can have extended periods of time with the absence of administration of long-term ERT administration. In some embodiments, the methods as disclosed herein enable flexibility in normal ERT regimens, in that extended breaks or withdrawal of administration of long-term ERT does not result in a clinical decline—that is, a subject remains clinically stable despite not having ongoing long-term ERT.

In some embodiments, the methods as disclosed herein encompass re-administration of ERT (herein referred to as “complementary ERT”) after an extended period of time of cessation of ERT administration, and enable flexibility in normal ERT regimen, as the continued production of GAA expressed by the AAV permits ERT flexibility. In some embodiments, the complementary ERT is pulse administration of ERT, as disclosed herein. In some embodiments, the complementary ERT is at less frequent intervals, or at a lower dose, or at irregular doses, or at irregular intervals as compared to the prior administration of long-term ERT.

Accordingly, the methods as disclosed herein provide significant advantages to subjects with Pompe disease, including but not limited to reducing or eliminating the rigorous and arduous weekly, or every-other week infusions of long-term rhGAA ERT treatment, which are significantly time-consuming and geographically limiting, and hinders a patient with Pompe disease from travelling for prolonged periods from areas where their ERT infusions are administered. Additionally, as disclosed herein, the absence of ERT administration also reduces any side effects due to anti-rhGAA antibodies against the ERT, and also circumvents the need for administration of immune suppressants normally co-administered with the ERT. As such, the methods to treat Pompe disease as disclosed here leads to greater flexibility in Pompe treatment and an improvement in quality of life and lifestyle of subjects with Pompe disease.

Accordingly, in one embodiment, the technology relates to a method of treating Pompe disease in a subject, comprising administering to the subject a pharmaceutical composition comprising a recombinant adeno-associated virus (AAV) vector comprising in its genome, a heterologous nucleic acid sequence encoding an alpha-glucosidase (GAA) polypeptide in expressible form wherein the heterologous nucleic acid is operatively linked to a liver-specific promoter, in the absence of administration of long-term GAA enzyme replacement therapy (ERT) for an extended period of time (e.g., ERT administration can be withdrawn or stopped at about 24, or at about 26 weeks, or earlier than 24- or 26 weeks, after administration of the recombinant AAV). In some embodiments, the dosage of the recombinant AAV comprising nucleic acid encoding GAA polypeptide ranges from 1.0E11 vg/kg and 5.0E13 vg/kg, and in some embodiments, the GAA is expressed to a level that the subject obtains a blood serum level of GAA expressed by the AAV at a pharmaceutical activity range from 160 to ≤2,260 nmol/mL/hr, 165 to ≤2,260 nmol/ml/hr, 175 to ≤2,260, 180 to ≤2,260, 185 to ≤2,260, 189 to ≤2,260 of at least within two weeks of administration. In some embodiments, the dosage of the AAV expressing a GAA polypeptide ranges from 11.0E11 vg/kg and 5.0E13 vg/kg, and in some embodiments, is no more than 4.0E¹² vg/kg, and in some embodiments, the GAA is expressed to a level that the subject obtains a blood serum level of GAA expressed by the AAV at a pharmaceutical activity range from 189 to ≤2,260 nmol/mL/hr of at least within two weeks of administration. In some embodiments, the dosage of the AAV expressing GAA is no more than 4.0E¹² vg/kg, and in some embodiments, the GAA is expressed to a level that the subject obtains a blood serum level of GAA expressed by the AAV at a pharmaceutical activity range from 189 to ≤2,260 nmol/mL/hr of at least within two weeks of administration.

In some embodiments, the dosage of AAV expressing GAA is no more than 5.0E¹¹ vg/kg. In some embodiments, the dosages range from 1.0E⁹ vg/kg to 5.0E¹¹ vg/kg.

In some embodiments, the dosage of AAV expressing GAA is no more than 5.0E13 vg/kg. In some embodiments, the dosages range from 1.0E9 vg/kg or 5.0E13 vg/kg.

In particular, the technology described herein relates to the discovery that a single infusion of a rAAV vector expressing human acid alpha-glucosidase (GAA) can be a stand-alone replacement for repeated infusions of enzyme replacement therapy (ERT) with recombinant human GAA protein (rhGAA). The inventors demonstrate that a one-time administration of AAV expressing GAA leads to long-term transduction of a normal GAA gene into hepatocytes and continuous constitutive expression of GAA in the systemic circulation. Accordingly, the inventors demonstrate herein that administration of a composition comprising AAV expressing hGAA can replace the biweekly exogenous administration of ERT that subjects with Pompe disease normally receive. That is, the inventors have demonstrated herein that subjects with Pompe that are administered a AAV expressing hGAA as disclosed herein can have long term cessation of ERT.

In particular, described herein is a method of treating Pompe in a subject in need thereof by administering the subject a composition comprising a AAV vector expressing the α-glucosidase (GAA) protein, where the subject is not being concurrently administered a GAA enzyme replacement therapy. In some embodiments, the technology relates to a method of administering a AAV expressing GAA where the subject can be withdrawn from a GAA enzyme replacement therapy (ERT) for an extended period of time, e.g., at least 3 months, at least 4 months, at least 5 months, at least 1 year, at least 1%₂ years and points in between 6 months or longer. In some embodiments, the subject is withdrawn from ERT on the day of, or shortly before administration of a AAV expressing GAA, and is clinically stable with respect to at least one or more, as disclosed herein. In some embodiments, the subject is withdrawn from ERT at any time between 1-2 days before or after administration, and about 6-months after administration of a AAV expressing GAA, and is clinically stable with respect to at least one or more Pompe symptoms for at least 6 months, as disclosed herein.

Additionally, the inventors have also discovered that Pompe patients administered a AAV expressing GAA according to the methods and dose ranges as disclosed herein, there is minimal immune response to the GAA protein expressed by the AAV. According, in some embodiments, there is minimal, or no need for immune modulation or administration of immune suppressants at the time of, or before, or after the administration of the AAV to the subject, and therefore normal immune suppressants protocols which are typically administered when a subject is administered a viral vector, or undergoing gene therapy are not required.

Accordingly, in all embodiments herein, the method to treat Pompe comprises, or consists essentially of, or consists of, administering an AAV vector expressing GAA as disclosed herein, in the absence of administration of ERT for Pompe, and also in the absence of immune modulation. In some embodiments, the subject has late onset Pompe Disease (LOPD) or infantile-onset Pompe disease.

In all aspects disclosed herein, the AAV, that comprise a nucleotide sequence containing inverted terminal repeats (ITRs), a promoter, a heterologous gene, a poly-A tail and potentially other regulator elements for use to treat a Pompe disease, e.g., late onset Pompe disease (LOPD), wherein the heterologous gene is GAA, and wherein the vector, e.g., rAAV can be administered to a patient in a therapeutically effective dose that is delivered to the appropriate tissue and/or organ for expression of the heterologous GAA gene and treatment of the disease, e.g., Pompe disease.

In some embodiments, a subject administered a AAV vector expressing GAA as disclosed herein, can, after an initial period of withdrawal of the administration of long-term ERT for an extended period of time, be administered complementary ERT, where the complementary ERT is administered after about 6-months, or about 1 year, or longer than a year of cessation of the long-term ERT. Stated differently and without wishing to be bound by theory, the technology disclosed herein relates to a method whereby a subject with Pompe disease who is administered a AAV vector expressing GAA as disclosed herein, can have breaks or “holidays” from the normal long-term ERT administration. That is, according to the methods as disclosed herein, a subject administered an AAV vector expressing GAA as disclosed herein can have extended periods of time with the absence of administration of long-term ERT administration. In some embodiments, the methods as disclosed herein enable flexibility in normal ERT regimens, in that extended breaks or withdrawal of administration of long-term ERT does not result in a clinical decline—that is, a subject remains clinically stable despite not having ongoing long-term ERT.

In some embodiments, the methods as disclosed herein encompass re-administration of ERT (herein referred to as “complementary ERT”) after an extended period of time of cessation of ERT administration, and enable flexibility in normal ERT regimen, as the continued production of GAA expressed by the AAV permits ERT flexibility. In some embodiments, the complementary ERT is pulse administration of ERT, as disclosed herein. In some embodiments, the complementary ERT is at less frequent intervals, or at a lower dose, or at irregular doses, or at irregular intervals as compared to the prior administration of long-term ERT.

Accordingly, the methods as disclosed herein provide significant advantages to subjects with Pompe disease, including but not limited to reducing or eliminating the rigorous and arduous weekly, or every-other week infusions of long-term rhGAA ERT treatment, which are significantly time-consuming and geographically limiting, and hinders a patient with Pompe disease from travelling for prolonged periods from areas where their ERT infusions are administered. Additionally, as disclosed herein, the absence of ERT administration also reduces any side effects due to anti-rhGAA antibodies against the ERT, and also circumvents the need for administration of immune suppressants normally co-administered with the ERT. As such, the methods to treat Pompe disease as disclosed here leads to greater flexibility in Pompe treatment and an improvement in quality of life and lifestyle of subjects with Pompe disease.

Accordingly, in one embodiment, the technology relates to a method of treating Pompe disease in a subject, comprising administering to the subject a pharmaceutical composition comprising a recombinant adeno-associated virus (AAV) vector comprising in its genome, a heterologous nucleic acid sequence encoding an alpha-glucosidase (GAA) polypeptide in expressible form wherein the heterologous nucleic acid is operatively linked to a liver-specific promoter, in the absence of administration of long-term GAA enzyme replacement therapy (ERT) for an extended period of time (e.g., ERT administration can be withdrawn or stopped at about 24, or at about 26 weeks, or earlier than 24- or 26 weeks, after administration of the recombinant AAV). In some embodiments, the dosage of the recombinant AAV comprising nucleic acid encoding GAA polypeptide ranges from 1.0E9 vg/kg to 5.0E13 vg/kg, and in some embodiments, the GAA is expressed to a level that the subject obtains a blood serum level of GAA expressed by the AAV at a pharmaceutical activity range from 160 to ≤2,260 nmol/mL/hr, 165 to ≤2,260 nmol/ml/hr, 175 to ≤2,260, 180 to ≤2,260, 185 to ≤2,260, 189 to ≤2,260 of at least within two weeks of administration. In some embodiments, the dosage of the AAV expressing GAA is in the range of 1.0E9 vg/kg and 5.0E13 vg/kg, and in some embodiments, the GAA is expressed to a level that the subject obtains a blood serum level of GAA expressed by the AAV at a pharmaceutical activity range from 189 to ≤2,260 nmol/mL/hr of at least within two weeks of administration. In some embodiments, the dosage of the AAV expressing GAA is in the range of 1.0E9 vg/kg and 5.0E13 vg/kg, and in some embodiments, the GAA is expressed to a level that the subject obtains a blood serum level of GAA expressed by the AAV at a pharmaceutical activity range from 189 to ≤2,260 nmol/mL/hr of at least within two weeks of administration.

In particular, the technology described herein relates to the discovery that a single infusion of a rAAV vector expressing human acid alpha-glucosidase (GAA) can be a stand-alone replacement for repeated infusions of enzyme replacement therapy (ERT) with recombinant human GAA protein (rhGAA). The inventors demonstrate that a one-time administration of AAV expressing GAA leads to long-term transduction of a normal GAA gene into hepatocytes and continuous constitutive expression of GAA in the systemic circulation. Accordingly, the inventors demonstrate herein that administration of a composition comprising AAV expressing hGAA can replace the biweekly exogenous administration of ERT that subjects with Pompe disease normally receive. That is, the inventors have demonstrated herein that subjects with Pompe that are administered a AAV expressing hGAA as disclosed herein can have long term cessation of ERT.

In particular, described herein is a method of treating Pompe in a subject in need thereof by administering the subject a composition comprising a AAV vector expressing the α-glucosidase (GAA) protein, where the subject is not being concurrently administered a GAA enzyme replacement therapy. In some embodiments, the technology relates to a method of administering a AAV expressing GAA where the subject can be withdrawn from a GAA enzyme replacement therapy (ERT) for an extended period of time, e.g., at least 3 months, at least 4 months, at least 5 months, at least 1 year, at least 1%₂ years and points in between 6 months or longer. In some embodiments, the subject is withdrawn from ERT on the day of, or shortly before administration of a AAV expressing GAA, and is clinically stable with respect to at least one or more, as disclosed herein. In some embodiments, the subject is withdrawn from ERT at any time between 1-2 days before or after administration, and about 6-months after administration of a AAV expressing GAA, and is clinically stable with respect to at least one or more Pompe symptoms for at least 6 months, as disclosed herein.

Additionally, the inventors have also discovered that Pompe patients administered a AAV expressing GAA according to the methods and dose ranges as disclosed herein, there is minimal immune response to the GAA protein expressed by the AAV. According, in some embodiments, there is minimal, or no need for immune modulation or administration of immune suppressants at the time of, or before, or after the administration of the AAV to the subject, and therefore normal immune suppressants protocols which are typically administered when a subject is administered a viral vector, or undergoing gene therapy are not required.

Accordingly, in all embodiments herein, the method to treat Pompe comprises, or consists essentially of, or consists of, administering an AAV vector expressing GAA as disclosed herein, in the absence of administration of ERT for Pompe, and also in the absence of immune modulation. In some embodiments, the subject has late onset Pompe Disease (LOPD) or infantile-onset Pompe disease.

The disclosure herein relates, in general, to a method to treat a subject with Pompe Disease, comprising administering to the subject with Pompe disease a pharmaceutical composition comprising, or consisting essentially of, a recombinant adenovirus associated (AAV) vector comprising in its genome, a heterologous nucleic acid sequence encoding a polypeptide comprising an alpha-glucosidase (GAA) polypeptide, wherein the heterologous nucleic acid is operatively linked to a liver-specific promoter, and wherein the subject is not administered a GAA enzyme replacement therapy (ERT) for an extended period of time, or can have extended breaks from ERT. In one embodiment, ERT is continued, but at least one of: dosage or frequency is reduced.

Without wishing to be bound by theory, the inventors have discovered a method to treat Pompe disease with AAV-mediated delivery of GAA to the subject, where the rAAV expresses GAA to a steady state shortly after administration, such that the subject could be withdrawn from ERT as early as, or at, or around the time of the rAAV administration, e.g., ERT can be withdrawn the day before administration, the same day of administration, or the day after administration, or within a week, or within 2-weeks of administration. As disclosed herein, a steady state of GAA expression by the rAAV as disclosed herein is a serum level of GAA at a pharmacological activity range from 189 to ≤2,260 nmol/mL/hr.

In some embodiments, the method to treat Pompe disease with rAAV expressing GAA as disclosed herein comprises administration of a therapeutically effective amount of a rAAV to result in a serum level of expressed hGAA within a pharmacological activity range of between 189 to 410 nmol/mL/hr, or 410 to ≤2,260 nmol/mL/hr.

In some embodiments, the method to treat Pompe disease with rAAV expressing GAA as disclosed herein comprises administration of a rAAV to result in a serum level of expressed hGAA within a range of 189 to ≤2,260 nmol/mL/hr, and where the subject achieves clinical stability of one or more symptoms of Pompe disease. Clinical stability includes a steady state in any one or more of the parameters: the 6 MWT (6-minute walk test), FVC (Forced vital capacity). In some embodiments, clinical stability refers to a stable level in either motor function (as determined by the 6 MWT) and/or pulmonary function (as determined by the FVC) in two consecutive assessments no less than 3-months apart. In some embodiments, a clinical stable level of motor function as determined by the 6 MWT is ≤12% decline, or less than a 43-meter decrease from baseline in two consecutive assessments no less than 3-months apart. Stated differently, a clinical stable level of motor function as determined by the 6 MWT position is within a 0-12% decline from a baseline level in two consecutive assessments no less than 3-months apart.

In some embodiments, a clinical stable level of pulmonary function as determined by the FVC % predicted in an upright position is ≤15% decrease from baseline in two consecutive assessments no less than 3-months apart. Stated differently, a clinical stable level of pulmonary function as determined by the % FVC predicted in an upright position is between 1-14% from a baseline in two consecutive assessments no less than 3-months apart.

In some embodiments, the baseline level of the 6 MWT or FVC is the level measured at or before administration of the rAAV expressing GAA. In some embodiments, the baseline level of the 6 MWT or FVC is the level measured at or before administration of the rAAV expressing GAA when the subject is concurrently administered GAA ERT. In some embodiments, the baseline level of the 6 MWT or FVC is the level measured at or before administration of the rAAV expressing GAA when the subject is withdrawn from GAA ERT. In some embodiments, the baseline level of the 6 MWT or, FVC is the level before withdrawing GAA ERT, e.g, at about 24 to about 26 weeks. In some embodiments, clinical stability is maintained between before ERT withdrawal and after ERT withdrawal of Pompe patients where the patients have received single administration of AAV comprising nucleic acid encoding GAA administrated at the the time of ERT administration, before ERT administration, or, after ERT administration. Clinical stability is maintained indicate that 6 MWT and or, FVC are within the ranges from baseline as described herein.

In some embodiments, the method to treat Pompe disease with rAAV expressing GAA as disclosed herein comprises administration of an amount of rAAV to result in a reduction of glycogen levels in one or more tissues to within a normal range, where the normal range is the glycogen levels in the comparative tissue of a subject without Pompe disease.

### A. AAV-hGAA Dosages

In some embodiments, the methods disclosed herein relate to human subjects can be administered a rAAV expressing GAA as disclosed herein at a dose in the range of 1.0E11 vg/kg and 5.0E13 vg/kg. In some embodiments, ERT withdrawal can occur at the time of the administration of the rAAV expressing GAA, or occurring at about 24 or 26 weeks after recombinant AAV administration. In some embodiments, there can be a therapeutic correction of disease pathophysiology with administration of the rAAV expressing GAA as disclosed herein, even in the absence of ERT, and also protection against immune response to the expressed hGAA e.g, as measured by the antibodies against the expressed hGAA, therefore demonstrating that subjects with Pompe disease that are administered a AAV expressing hGAA as disclosed herein can have long term cessation of ERT, which is further discussed, e.g., in International Patent Application No. PCT/US2023/013713, which is incorporated herein by reference in its entirety.

In some embodiments the dose of the a rAAV vector or rAAV genome to be administered to the subject according to the method to treat Pompe Disease as disclosed herein depends upon the mode of administration, the promoter used, the signal peptide used, the severity of the Pompe disease or other condition to be treated and/or prevented, the individual subject's condition, the particular virus vector or capsid, the liver-specific promoter being used and the nucleic acid to be delivered, including but not limited to, nucleic acid encoding the signal peptide attached to the 5′ of the nucleic acid encoding expressible GAA polypeptide, and the like, and can be determined in a routine manner.

In some embodiments, the therapeutically effective amount of the rAAV vector expressing GAA is an amount that results in a serum GAA concentration at steady state similar to the GAA pharmacological activity achieved by long term GAA ERT (e.g within 5%, 10%, 20% of such levels). For example, a target GAA serum concentration at steady state ranging from about 160 to ≤2,260 nmol/mL/hr, from about 189 to ≤2,260 nmol/mL/hr, or rangin from 410 to ≤2,260 nmol/mL/hr. In some embodiments, the dose of the rAAV vector expressing GAA is a therapeutically effective amount to achieve a target GAA serum concentration at steady state that confers pharmacological activity ranges from 189 to ≤2,260 nmol/mL/hr. In some embodiments, the dose of the rAAV vector expressing GAA is a therapeutically effective amount to increase the tissue GAA levels in the subject to >0.30 μmol 4MU/min/gram of tissue, where the normal range of tissue GAA content in a subject without Pompe disease is 0.36±/0.13 μmol 4MU/min/gram of tissue. In some embodiments, the dose of the rAAV vector expressing GAA is a therapeutically effective amount to increase the tissue GAA levels in the subject to between 0.25 to 0.4 μmol 4MU/min/gram of tissue. In some embodiments, the dose of the rAAV vector expressing GAA is a therapeutically effective amount to result in a normal tissue GAA content of about 0.36 μmol 4MU/min/gram of tissue, e.g. about 0.25, or about 0.26, or about 0.27, or about 0.28, or about 0.29, or about 0.30, or about 0.31, or about 0.32, or about 0.33, or about 0.34, or about 0.35, or about 0.36, or about 0.37, or about 0.38, or about 0.39, or about 0.40 μmol 4MU/min/gram of tissue.

In some embodiments, the dose of the rAAV vector expressing GAA is a therapeutically effective amount to increase the tissue GAA levels in the subject to between 0.1-0.5 μmol 4MU/min/gram of tissue. In some embodiments, the dose of the rAAV vector expressing GAA is a therapeutically effective amount to result in a normal tissue GAA content of about greater than 0.36 μmol 4MU/min/gram of tissue. In some embodiments, the dose of the rAAV vector expressing GAA is a therapeutically effective amount to increase tissue GAA content or levels in the subject within the range 0.2-0.4 mol 4MU/min/gram of tissue. In some embodiments, the dose of the rAAV vector expressing GAA is a therapeutically effective amount to increase tissue GAA content in the subject to within 40%, or within 30%, or within 20%, or within 10%, or within 9%, 8%, 7%, 6%, 5%, 4%, 3%, 2% or 1% of the normal muscle tissue GAA content of 0.36±0.13 (μmol 4MU/min/gram of tissue), where the GAA content of normal muscle tissue is a reference level of GAA in a subject without Pompe Disease. In some embodiments, the dose of the rAAV vector expressing GAA is a therapeutically effective amount to increase tissue GAA content in the subject greater than 0.1 mol 4MU/min/gram of tissue, where the normal range GAA content in subjects with Pompe disease is 0.05±0.04 μmol 4MU/min/gram of tissue). In some embodiments, the dose of the rAAV vector expressing GAA is a therapeutically effective amount to increase tissue GAA content in the subject more than 2-fold, or 3-fold, or 4-fold, or 5-fold, or 6-fold, or 7-fold, or 8-fold, or 9-fold, or 10-fold, or more than 10-fold of the level of GAA tissue content in the subject with Pompe. In some embodiments, the dose of the rAAV vector expressing GAA is a therapeutically effective amount to increase tissue GAA content in the subject to about 50%, or, about 40%, or about 30%, or about 20%, or about 10%, or about 9%, about 8%, about 7%, about 6%, about 5%, about 4%, about 3%, about 2% or about 1% of the level of GAA tissue content in the subject with Pompe. In some embodiments, the GAA activity in muscle is at least 1.5 fold, at least 2 fold, at least 3 fold, at least 5 fold, at least 8 fold, or at least 10 fold than the level prior to AAV administration. In some embodiments, the GAA activity in muscle is at least 1.5 fold, at least 2 fold, at least 3 fold, at least 5 fold, at least 8 fold, or at least 10 fold than the level after the long term ERT was withdrawn for at least about 24 weeks.

In some embodiments, the dose of the rAAV vector expressing GAA is a therapeutically effective amount to reduce the tissue glycogen levels in the subject within the range 0.25% wet tissue weight to about 1.5% wet tissue weight. In some embodiments, the dose of the rAAV vector expressing GAA is a therapeutically effective amount to reduce the muscle tissue glycogen levels in the subject to within 40%, or within 30%, or within 20%, or within 10%, or within 9%, 8%, 7%, 6%, 5%, 4%, 3%, 2% or 1% of the normal muscle tissue glycogen content of 0.99%±0.74 (% wet tissue weight), which is the normal muscle tissue glycogen content (measured as % wet tissue weight), of a subject that does not have Pompe disease.

In some embodiments, the dose of the rAAV vector expressing GAA is a therapeutically effective amount of GAA to exhibit an improvement in the therapeutic index of 3- to 5-fold.

In some embodiments, the dose of the rAAV vector expressing GAA is a therapeutically effective amount to result in the subject having clinically stable levels of hGAA at 10-weeks, or at least 20 weeks, or 30 weeks post AAV administration.

In an embodiment, as used herein, without limitation, the term “effective amount” is synonymous with “therapeutically effective amount”, “effective dose”, or “therapeutically effective dose.” In an embodiment, the effectiveness of a therapeutic compound disclosed herein to treat Pompe Disease can be determined, without limitation, by observing an improvement in an individual based upon one or more clinical symptoms, and/or physiological indicators associated with Pompe Disease. In an embodiment, an improvement in the symptoms associated with Pompe Disease can be indicated by a reduced need for a concurrent therapy.

In some embodiments, exemplary doses for achieving therapeutic effects of a rAAV vector expressing hGAA as disclosed herein is within the range of 1.0E⁹ vg/kg to 5.0E¹¹ vg/kg. In some embodiments, the dose administered to a subject is at least about 1.0E⁹ vg/kg, at least about 1.0E¹⁰ vg/kg, at least about 1.0E11 vg/kg, at least about 1.0E12 vg/kg, about 1.1E12 vg/kg, about 1.2E12 vg/kg, about 1.3E12 vg/kg, about 1.4E12 vg/kg, about 1.5E12 vg/kg, about 1.6E12 vg/kg, about 1.7E12 vg/kg, about 1.8E12 vg/kg, about 1.9E12 vg/kg, about 2.0E12 vg/kg, about 3.0E12 vg/kg, about 4.0E12 vg/kg, about 5.0E12 vg/kg, about 6.0E12 vg/kg, about 7.0E12 vg/kg, about 8.0E12 vg/kg, about 9.0E12 vg/kg, about 1.0E13 vg/kg, about 1.2E13 vg/kg, about 1.2E13 vg/kg, about 1.2E13 vg/kg, about 1.3E13 vg/kg, about 1.4E13 vg/kg, about 1.5E13 vg/kg, about 1.6E13 vg/kg, about 1.7E13 vg/kg, about 1.8E13 vg/kg, about 1.9E13 vg/kg, about 2.0E13 vg/kg, about 3.0E13 vg/kg, about 4.0E13 vg/kg, about 5.0E13 vg/kg, about 6.0E13 vg/kg, about 7E13 vg/kg, about 8E13 vg/kg, about 9E13 vg/kg, or about 10E14 vg/kg. In some embodiments the rAAV administration is accompanied with immunomodulators, e.g, prednisone, methotrexate or, a combination thereof. In some embodiments the rAAV of the invention is packaged within AAV XL 32 or AAV XL 32.1 capsid.

In preferred embodiments, exemplary doses for achieving therapeutic effects according to the methods as disclosed herein are titers of at between 1.2E12 and 4.0E12 vg/kg, for example, least about 1.0E12 vg/kg, about 1.1E12 vg/kg, about 1.2E12 vg/kg, about 1.3E12 vg/kg, about 1.4E12 vg/kg, about 1.5E12 vg/kg, about 1.6E12 vg/kg, about 1.7E12 vg/kg, about 1.8E12 vg/kg, about 1.9E12 vg/kg, about 2.0E12 vg/kg, about 2.1E12 vg/kg, about 2.2E12 vg/kg, about 2.3E12 vg/kg, about 2.4E12 vg/kg, about 2.5E12 vg/kg, about 2.6E12 vg/kg, about 2.7E12 vg/kg, about 2.8E12 vg/kg, about 2.9E12 vg/kg, about 3.0E12 vg/kg, about 3.1E12 vg/kg, about 3.2E12 vg/kg, about 3.3E12 vg/kg, about 3.4E12 vg/kg, about 3.5E12 vg/kg, about 3.6E12 vg/kg, about 3.7E12 vg/kg, about 3.8E12 vg/kg, about 3.9E12 vg/kg, about 4.0E12 vg/kg.

In preferred embodiments, exemplary doses for achieving therapeutic effects according to the methods as disclosed herein are titers of at between 1.0E11 vg/kg and 5.0E13 vg/kg, for example, 1.0E11 vg/kg, 1.1E11 vg/kg, 1.2E11 vg/kg, 1.3E11 vg/kg, 1.4E11 vg/kg, 1.5E11 vg/kg, 1.6E11 vg/kg, 1.7E11 vg/kg, 1.8E11 vg/kg, 1.9E11 vg/kg, about 1.0E12 vg/kg, about 1.1E12 vg/kg, about 1.2E12 vg/kg, about 1.3E12 vg/kg, about 1.4E12 vg/kg, about 1.5E12 vg/kg, about 1.6E12 vg/kg, about 1.7E12 vg/kg, about 1.8E12 vg/kg, about 1.9E12 vg/kg, about 2.0E12 vg/kg, about 3.0E12 vg/kg, about 4.0E12 vg/kg, about 5.0E12 vg/kg, about 6.0E12 vg/kg, about 7.0E12 vg/kg, about 8.0E12 vg/kg, about 9.0E12 vg/kg, about 1.0E13 vg/kg, about 1.2E13 vg/kg, about 1.2E13 vg/kg, about 1.2E13 vg/kg, about 1.3E13 vg/kg, about 1.4E13 vg/kg, about 1.5E13 vg/kg, about 1.6E13 vg/kg, about 1.7E13 vg/kg, about 1.8E13 vg/kg, about 1.9E13 vg/kg, about 2.0E13 vg/kg, about 3.0E13 vg/kg, about 4.0E13 vg/kg, about 5.0E13 vg/kg.

In some embodiments, a rAAV vector expressing hGAA as disclosed herein useful for the methods to treat Pompe Diseases, exemplary doses for achieving therapeutic effects are titers of at least about 1.0E12 to 4.0E12 vg/kg, or about 1.2E12 to 3.0E12 vg/kg, or about 1.2E12 to 2.5E12 vg/kg, or about 2.5E12 to 4.0E12 vg/kg.

In some embodiments, the dosage may be modified by a person of ordinary skill in the art, e.g., the dose administered can be lower than 1.0E12 vg/kg, or lower than about 5.0E11 vg/kg where a stronger promoter than the LSP of SEQ ID NO: 97 is operatively linked to the nucleic acid encoding GAA. In contrast, in alternative embodiments, the dosage may be modified by a person of ordinary skill in the art, e.g., the dose of the rAAV vector administered can be higher than about 1.6E12 vg/kg, or higher than about 5.0E12 vg/kg when a weaker liver-specific promoter than the LSP of SEQ ID NO: 97 used in the AAV8-LSPhGAA vector is operatively linked to the nucleic acid encoding GAA. Exemplary doses for achieving therapeutic effects are titers of at least about 1.0E⁵, 1.0E⁶, 1.0E⁷, 1.0E⁸, 1.0E⁹, 1.0E¹⁰, 1.0E¹¹, 1.0E¹² vg/kg, optionally about 1.0E¹⁰ to about 1.0E¹² transducing units (vg/kg), and optionally does not exceed about 4.0E¹² vg/kg or optionally is about 3.0E¹² transducing units (vg/kg).

In one embodiment, no percentage of the administered dose of rAAV vector expressing hGAA as disclosed herein is retained in the liver following administration, e.g., at least 1, 2, 3, 4 weeks or more following administration.

In one embodiment, less than 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% of the administered dose of rAAV vector expressing hGAA as disclosed herein is retained in the liver following administration, e.g., at least 1, 2, 3, 4 weeks or more following administration.

In one embodiment, less than 1.0E⁹ vg/kg to 5.0E¹¹ vg/kg of the administered rAAV vector expressing hGAA as disclosed herein is retained in the liver following administration, e.g., at least 1, 2, 3, 4 weeks or more following administration.

In one embodiment, less than 1.0E⁹ vg/kg, 1.0E¹⁰ vg/kg, 1.0E11 vg/kg, 1.0E12 vg/kg, 1.1E12 vg/kg, 1.2E12 vg/kg, 1.3E12 vg/kg, 1.4E12 vg/kg, 1.5E12 vg/kg, 1.6E12 vg/kg, 1.7E12 vg/kg, 1.8E12 vg/kg, 1.9E12 vg/kg, 2.0E12 vg/kg, 3.0E12 vg/kg, 4.0E12 vg/kg, 5.0E12 vg/kg, 6.0E12 vg/kg, 7.0E12 vg/kg, 8.0E12 vg/kg, 9.0E12 vg/kg, 1.0E13 vg/kg, 1.2E13 vg/kg, 1.2E13 vg/kg, 1.2E13 vg/kg, 1.3E13 vg/kg, 1.4E13 vg/kg, 1.5E13 vg/kg, 1.6E13 vg/kg, 1.7E13 vg/kg, 1.8E13 vg/kg, 1.9E13 vg/kg, 2.0E13 vg/kg, 3.0E13 vg/kg, 4.0E13 vg/kg, or 5.0E13 vg/kg of the administered rAAV vector expressing hGAA as disclosed herein is retained in the liver following administration, e.g., at least 1, 2, 3, 4 weeks or more following administration.

In a further embodiment, administration of rAAV vector or rAAV genome according to the methods as disclosed herein to treat a subject with Pompe disease can result in production of a GAA protein with a circulatory half-life of 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, 1 day, 2 days, 3 days, 4 days, 5 days, 6 days, 7 days, 1 week, 2 weeks, 3 weeks, 4 weeks, one month, two months, three months, four months or more.

In some embodiments, the methods for treatment of Pompe as disclosed herein relate to a single dose of a rAAV expressing hGAA is used to treat a subject in a single administration. However, in some embodiments, the dose of rAAV to be administered can be given to the subject in multiple administrations, e.g., a dose of rAAV can be divided into sub-doses and administered in multiple administrations.

In some embodiments, it is envisioned that the methods for treatment of Pompe as disclosed herein can comprise multiple administrations of a single dose of a rAAV expressing hGAA, that is, the subject can be treated with a booster administration (i.e., a second, third, fourth, etc.) of a rAAV expressing hGAA after a defined period of time after the initial or first administration. The dose of a booster administration (i.e., 2^{nd}, 3^{rd}, 4^{th}, or 5^{th} etc.) can be the same dose (amount) of rAAV-hGAA administered in the first administration, or can be a higher dose, or a lower dose, depending on the factors above, including, but not limited to, a therapeutically effective dose to achieve any one or more of (i) serum GAA levels indicating steady state of GAA expression, (ii) reduced glycogen levels and/or, maintained glycogen levels within normal range in the muscle, and (iii) one or more Pompe symptoms, including muscle function and/or pulmonary function within clinically stable levels. As disclosed herein, a steady state of GAA expression by the rAAV as disclosed herein is a serum level of GAA at a pharmacological activity range from 165 to ≤2260 nmol/ml/hr or, from 189 to ≤2,260 nmol/mL/hr. Stability of one or more symptoms of Pompe disease can be determined by the clinical stability parameters as disclosed herein, and includes a steady state in the 6 MWT (6-minute walk test) and/or FVC (Forced vital capacity) in two consecutive assessments no less than 3-months apart as disclosed herein. In some embodiments, a clinical stable level of motor function as determined by the 6 MWT is ≤12% decline, or less than a 43-meter decrease from baseline in two consecutive assessments no less than 3-months apart. In some embodiments, a clinical stable level of pulmonary function as determined by the FVC % predicted in an upright position is ≤15% decrease from baseline in two consecutive assessments no less than 3-months apart.

In an embodiment, the time period of between administration of a first dose, and a subsequent dose (i.e., a booster dose) of a rAAV vector according to the methods for treatment of Pompe as disclosed herein is selected from any of the following: about 4 months, about 6 months, about 7 months, about 8 months, about 9 months, about 12 months, about 18 months, about 24 months, or about 3 years, about 4 years, about 5 years, or more than 5 years

In another embodiment, administration of a rAAV vector or rAAV genome as disclosed herein for the treatment of Pompe Disease results in an increase in weight by, e.g., at least 0.5 pounds, at least 1 pound, at least 1.5 pounds, at least 2 pounds, at least 2.5 pounds, at least 3 pounds, at least 3.5 pounds, at least 4 pounds, at least 4.5 pounds, at least 5 pounds, at least 5.5 pounds, at least 6 pounds, at least 6.5 pounds, at least 7 pounds, at least 7.5 pounds, at least 8 pounds, at least 8.5 pounds, at least 9 pounds, at least 9.5 pounds, at least 10 pounds, at least 10.5 pounds, at least 11 pounds, at least 11.5 pounds, at least 12 pounds, at least 12.5 pounds, at least 13 pounds, at least 13.5 pounds, at least 14 pounds, at least 14.5 pounds, at least 15 pounds, at least 20 pounds, at least 25 pounds, at least 30 pounds, at least 50 pounds.

In another embodiment, an AAV GAA of any serotype, as disclosed herein for the treatment of Pompe Disease results in an increase in weight by, e.g., from 0.5 pounds to 50 pounds, from 0.5 pounds to 30 pounds, from 0.5 pounds to 25 pounds, from 0.5 pounds to 20 pounds, from 0.5 pounds to 15 pounds, from 0.5 pounds to ten pounds, from 0.5 pounds to 7.5 pounds, from 0.5 pounds to 5 pounds, from 1 pound to 15 pounds, from 1 pound to 10 pounds, from 1 pound to 7.5 pounds, form 1 pound to 5 pounds, from 2 pounds to ten pounds, from 2 pounds to 7.5 pounds.

### B. Withdrawal of Long-Term ERT

Treatment of Pompe disease is normally by administration of long-term enzyme replacement therapy (ERT) with recombinant human acid α-glucosidase (rhGAA) and has previously reported to prolong survival of both LOPD and IOPD patients through improvement in pulmonary and muscle function. However, Schoser et al, report that after a period of stabilization, both these parameters continue to decline over time (see Schoser et al., 2017 Neurol, 264: 621-30). Additionally, ERT with recombinant GAA protein has numerous disadvantages, including but not limited to, short-half life of the administered recombinant GAA in the blood, lack of efficient skeletal muscle uptake, potential for high titer antibody response and even some patients failing to respond to ERT, and rigorous administration of a recombinant GAA infusion every 2 weeks, that can take between 5-8 hours. Moreover, while disease progression is slowed compared with untreated subjects, the benefits of ERT may not be long-lasting, and many patients die or remain weak despite treatment compliance (Tarnopolsky et al. 2016 Can J Neurol Sci, 43: 472-85).

The waning efficacy in subjects receiving recombinant GAA protein ERT is partially explained by an immune response to ERT where high, sustained anti-GAA antibody titers (HSAT) are formed. Subjects with HSAT demonstrated greatly increased mortality, in comparison with patients who formed no or only low titer antibodies (Banugaria et al. 2011). Furthermore, the ability to prevent antibody formation in subjects at risk for HSAT with immunosuppression, which significantly prolonged survival, confirmed the clinical relevance of HSAT (Mendelsohn et al. 2009; Banugaria et al. 2013; Kazi et al. 2017).

Subjects with Pompe's disease are considered as cross-reactive immunologic material (CRIM) positive if they have residual GAA enzyme activity and CRIM negative if no residual GAA activity is detected. Based on pooled clinical studies data, 28% of Pompe's disease cases are infantile-onset, of which about 85% are classic infantile-onset and three quarters of those are CRIM positive (Kemper, Comeau, and Green 2013). Determination of CRIM status in newly diagnosed IOPD patients is important since it allows for the institution of preventive immunomodulatory measures before beginning ERT, thus offsetting the worst damaging effects of HSAT. CRIM-negative Pompe disease subjects produced HSAT and demonstrated markedly reduced efficacy from ERT with rhGAA (Amalfitano et al. 2001). In the first pilot study of ERT in Pompe disease using Chinese hamster ovary cell-derived rhGAA, the two subjects who were CRIM-negative produced higher titers of anti-GAA antibodies than the third subject who was CRIM-positive. This corresponded with a markedly reduced efficacy of ERT in the CRIM-negative subjects. The relevance of antibody formation to efficacy of therapy in Pompe disease has been emphasized by the poor response of CRIM-negative subjects to ERT, which correlated with the onset of HSAT (Kishnani et al. 2010).

While no LOPD subjects are CRIM negative, some mount high antibody responses to rhGAA capable of interfering with optimal efficacy of ERT (Patel et al. 2012; de Vries et al. 2017; “LUMIZYME Prescribing Information” 2014). In addition, all Pompe subjects mount some level of anti-GAA antibody response with unknown effects on ERT efficacy.

In some embodiments, the rAAV vectors expressing a GAA polypeptide as disclosed herein can be used in methods to treat subjects with Pompe disease, and comprises administering a AAV expressing hGAA as disclosed herein that enables the subject to have an extended period of cessation of the administration of long-term ERT. In particular, rAAV vectors expressing a GAA polypeptide as disclosed herein can be administered to a subject with Pompe disease that enables them to have the ability to reduce, or eliminate the clinical need for long-term hGAA ERT administration for an extended period of time.

Accordingly, another aspect of the technology disclosed herein relates to a method to treat Pompe Disease by administrating to the subject with Pompe disease a composition comprising a rAAV vector expressing a GAA polypeptide as disclosed herein, where in some embodiments, the methods enable subjects with Pompe disease to withdraw from, or stop long-term administration of recombinant human GAA (rhGAA) ERT, which is normally administered on a weekly or every-other week regimen. In some embodiments, the methods disclosed herein enable a subject with Pompe disease to take breaks from the normal ERT regimen for extended period of time (e.g., extended periods of ERT cessation) if the subject is administered a specific dose of AAV vector expressing a GAA polypeptide as disclosed herein. In some embodiments, withdrawal of the administration of long-term ERT begins at about the time of administration of the AAV vector to the subject (e.g., the day before, the day of, or the day after), or in some embodiments, withdrawal of the administration of long-term ERT can occur at about 24 weeks, or anywhere within about 24 weeks to about 26 weeks after administration of the AAV vector.

As disclosed herein, “long-term ERT” refer to the standard-of-care (SOC) treatment for a subject with Pompe disease, including IOPD and LOPD, and is normally a regimen of intravenous administration of recombinant human alglucisudease alfa protein (rhGAA) to the subject on a regular and frequent basis, e.g., every week or every 2 weeks, without any breaks in the regimen, and where the administered rhGAA protein provides an exogenous source of GAA. MYOZYME® (alglucosidase alfa) which was first US approved product (2006) for the treatment of Pompe disease and LUMIZYME® (alglucosidase alfa) which was approved in 2010 are exemplary current standard-of-care (SOC) treatments for infantile-onset and late-onset Pompe patients. The normal long-term ERT administration regimen is intravenously administration of Alglucosidase alfa every 2 weeks as an infusion at a dose of 20 mg/Kg (LUMIZYME Prescribing Information 2014).

As disclosed herein, in some embodiments, the methods as disclosed herein by administering a AAV expressing hGAA as disclosed herein enable the withdrawal or cessation of administration of long-term ERT for an extended period of time. In some embodiments, the extended period of time is at least about 3-months, or at least about 6-months, or at least about 1 year, or longer than 1 year.

As disclosed herein, “extended period” of time, as referred to in reference to time period that administration of long-term ERT is stopped, refers to a time period that is longer than 1 month, and in some embodiments is a time period longer than if up to 5 administrations of ERT are missed.

Accordingly, in some embodiments, the methods to treat a subject with Pompe Disease with a AAV expressing hGAA as disclosed herein comprises administering to the subject a pharmaceutical composition comprising a AAV expressing GAA and where the subject is not administered long-term GAA enzyme replacement therapy (ERT) for an extended period of time. In some embodiments, the cessation or withdrawal of administration of long-term ERT occurs anywhere between 1-2 days of administration and at least 24 weeks after the administration of the AAV-GAA vector. That is, in some embodiments, the subject being treated can stop the administration of ERT on the day of, or the day before or after administration of AAV-GAA. In some embodiments, the subject being treated according to the methods as disclosed herein can stop ERT after about 1 week, or about 2 weeks, or about 3 weeks, or about 1 month, or about 2 months, or about 3 months, or about 4 months, or about 5 months, or about 6 months after the administration of the AAV-GAA.

The exact timeframe for stopping ERT, or for ERT cessation, by each subject according to the methods as disclosed herein can be determined by an ordinary skilled practitioner, but without wishing to be limited by theory, encompassed herein is a method to treat a subject with Pompe disease by administering a AAV expressing hGAA as disclosed herein, where ERT is stopped at time point that the serum GAA levels achieved from expression by the AAV-hGAA is near or about a serum level of within a pharmacological activity range of at least 165 nmol/ml/hr or, of at least 189 nmol/ml/hr, for example, between 189 to ≤2,260 nmol/mL/hr. In some embodiments, encompassed herein is a method where ERT is stopped at time point that the serum GAA levels achieved from expression by the AAV-hGAA is within 50%, or within 60%, or within 70% or within 80% of a serum level of within a pharmacological activity range of between 189 nmol/mL/hr. In some embodiments, encompassed herein is a method to treat Pompe disease by administering a AAV expressing a hGAA polypeptide as disclosed herein, where ERT is stopped at time point that the serum GAA levels achieved from expression by the AAV-hGAA is within 50%, or within 60%, or within 70% or within 80% of a serum level of within a pharmacological activity range of between 165 to about 2000 nmol/mL/hr.

In some embodiments, encompassed herein is a method to treat Pompe disease by administering a AAV expressing a hGAA polypeptide as disclosed herein, where ERT is stopped at time point that the serum GAA levels achieved from a normal ERT regimen are replaced with a GAA serum level achieved from expression by the AAV-hGAA. For example, as the serum GAA levels due to the recombinant hGAA from the last ERT administration declines, there is a concurrent increase in serum GAA levels achieved from expression by the AAV-hGAA, so that ERT withdrawal or cessation does not result in a decline in clinical stability of one or more symptoms of Pompe disease in the subject, as measured by the 6 MWT or FVC according to the methods as disclosed herein. For illustration purposes only, in some embodiments, ERT withdrawal or cessation occurs when the administered AAV-hGAA results in the expressed GAA to achieve a serum GAA level for clinical stability of one or more symptoms of Pompe disease in the subject, for example, a clinical stable level of motor function as determined by the 6 MWT is ≤12% decline, or less than a 43-meter decrease from baseline in two consecutive assessments no less than 3-months apart, or a clinical stable level of pulmonary function as determined by the FVC % in an upright position is ≤15% decrease from baseline in two consecutive assessments no less than 3-months apart, therefore making superfluous the recombinant hGAA from the last ERT administration.

In some embodiments, the method to treat Pompe disease by administering a AAV expressing a hGAA polypeptide as disclosed herein enables long term cessation of ERT for a period of about 1 year, or about 15 months, or about 18 months, or about 24 months, or about 30 months or more than 30 months while maintaining clinical stable with one or more symptoms of Pompe disease, as measured by 6 MWT and/or % FVC, as disclosed herein.

Accordingly, the rAAV vectors encoding a GAA polypeptide as disclosed herein and the methods as disclosed herein provide significant advantages to subjects with Pompe disease, including but not limited to reducing or eliminating the rigorous and arduous weekly, or every-other week infusions of long-term rhGAA ERT treatment, which are significantly time-consuming and geographically limiting, and hinders a patient with Pompe disease from travelling for prolonged periods from areas where their ERT infusions are administered. Additionally, as disclosed herein, the absence of ERT administration also reduces any side effects due to anti-rhGAA antibodies against the ERT, and also circumvents the need for administration of immune suppressants normally co-administered with the ERT. As such, the methods to treat Pompe disease by administering a AAV expressing a hGAA polypeptide as disclosed herein leads to greater flexibility in Pompe treatment and an improvement in quality of life and lifestyle of subjects with Pompe disease.

### C. Administration of Complementary ERT

In some embodiments, the method to treat Pompe disease by administering a AAV expressing a hGAA polypeptide as disclosed herein enables a subject to have breaks or “holidays” from the normal regimen of administration long-term ERT. That is, according to the methods as disclosed herein, a subject who is administered an AAV vector expressing GAA as disclosed herein can take extended periods of time in the absence of administration of long-term ERT. Moreover, in some embodiments, a subject administered a AAV vector expressing GAA as disclosed herein, can, after an initial period of withdrawal of the administration of long-term ERT for an extended period of time, be administered complementary ERT, where the complementary ERT is administered after about 6-months, or about 1 year, or longer than a year of cessation of the long-term ERT. Accordingly, the methods to treat Pompe disease by administering a AAV expressing a hGAA polypeptide as disclosed herein enable flexibility in normal long-term ERT administration regimens, allowing both extended breaks or absence of administration of long-term ERT which does not result in a clinical decline—that is, a subject remains clinically stable despite not having ongoing long-term ERT administration for an extended period of time.

In some embodiments, the methods to treat Pompe disease by administering a AAV expressing a hGAA polypeptide as disclosed herein encompass re-administration of ERT (herein referred to as “complementary ERT”) after an extended period of time of cessation of ERT administration, and enable flexibility in normal ERT regimen, as the continued production of GAA expressed by the AAV permits include ERT flexibility. In some embodiments, the complementary ERT is pulse administration of ERT, as disclosed herein. In some embodiments, the complementary ERT is at less frequent intervals, or at a lower dose, or at irregular doses, or at irregular intervals as compared to the prior administration of long-term ERT.

In some embodiments, the methods to treat Pompe disease by administering a AAV expressing a hGAA polypeptide as disclosed herein encompass recommencement of ERT (herein referred to as “complementary ERT”) after an extended period of at least 6 months to about 1 year of absence of long-term ERT administration. In some embodiments, complementary ERT can be for a short-period of time, and can be followed by a second extended period of ERT administration cessation. In some embodiments, complementary ERT can be for a period of anywhere between 3 months to about 2 years, for example, about 3 months, 4 months, 5 months, 6 months, 7 months, 8 months, 9 months, 10 months, 11 months, or for about 1 year.

In some embodiments, the methods to treat Pompe disease by administering a AAV expressing a hGAA polypeptide as disclosed herein encompasses administering a rAAV expressing GAA according to as subject with Pompe, wherein administration of long-term ERT continues after administration of the recombinant AAV. However, the ERT is at a lower dose and/or frequency than before the administration of the recombinant AAV vector. For example, after administration of the AAV vector, long-term ERT can be administered every 3 weeks, once a month, bimonthly, once every 3 months, every 4 months, every 5 months, every 6 months for at least 24 weeks after administration of the AAV-GAA. Dosage of the long-term ERT can be reduced in one embodiment. In one method, a pulse administration regimen of long-term ERT after administration of the AAV vector can be used so that an irregular dosing schedule and/or amount can be used. As discussed herein, in some embodiments, administration of long-term ERT can be withdrawn at 24 weeks, or earlier as disclosed herein.

In some embodiments, due to continued expression of GAA by the administered AAV vector, the methods disclosed herein enable flexibility of administration of both long-term ERT or complementary ERT, such that if a subject plans to miss, or inadvertently or accidently misses one or more ERT administrations of a long-term ERT or complementary ERT regimen, the subject will maintain clinical stability. Currently, if ERT is missed, a much larger amount of ERT is needed to return to the same clinical level.

In some embodiments, the complementary ERT is at less frequent administration intervals, or at a lower dose, or at irregular doses, or at irregular administration intervals as compared to the prior administration of long-term ERT. For example, in some embodiments, the dose of rhGAA administered in a complementary ERT is less than 80%, less than 75%, less than 70%, less than 65%, less than 60%, less than 55%, less than 50%, less than 45%, less than 40%, less than 35%, less than 30%, less than 25%, less than 20%, less than 15%, less than 10%, less than 5%, less than 2%, or less than 1% of the normal dose of the rhGAA administered in a long-term ERT regimen.

In some embodiments, the complementary ERT is administered as pulse administration. In alternative embodiments, as a subject administered the rAAV vector compositions as disclosed herein can take breaks or interruptions from the regular dosing regimen of the long-term ERT administration or complementary ERT, where the long-term ERT or complementary ERT are administered by pulse administration. For example, in some embodiments, the administration of the long-term ERT or complementary ERT can be administered by pulsed administration. In certain embodiments, a subject administered the compositions can have pulsed administration of the long-term ERT or complementary ERT.

In certain embodiments, pulsed administration of the complementary ERT is suitable provided the subject has been administered the AAV vector composition as disclosed herein at a sufficient dose for continuous expression of GAA to maintain clinical stability and/or maintain a serum GAA level at or above 189 n mol/hr* (e.g., during the entire duration of the ERT break or “ERT holiday” where the regularly scheduled ERT is not administered). In certain embodiments, the methods disclosed herein allows a subject to undergo pulsed administration of complementary ERT for the lifetime of the subject.

In some embodiments, the regimen of administration of the complementary ERT can have intermittent breaks, where the administration of ERT is halted (e.g., the duration of the break or “ERT holiday” where the regimen of administration of ERT is halted).

In some embodiments, the methods encompass administration of complementary ERT by pulsed administration, where the pulsed administration of complementary ERT occurs least once a month, at least every other month, or at least every 6 months, or at least every year, or every other year. As the methods disclosed herein comprise administering a AAV vector at a sufficient dose for continuous expression of GAA in the subject and to maintain a serum GAA level at or above 189 n mol/hr*, if complementary ERT is administered, pulsed administration can substantially reduce the amount of ERT administered to the patient per dose or per total treatment regimen with an increased effectiveness, and allows for increased flexibility in a ERT administration regimen. This represents a significant saving in time, effort and expense and, more importantly, improved quality of life for Pompe patients, as well as a lower ERT dose which can lessens any side effects, including anti-GAA antibodies to the administered rhGAA protein.

In some embodiments, administration of complementary ERT is a pulsed administration. In certain embodiments, a pulsed administration comprises administering complementary ERT for about 8 weeks, followed by not administering complementary ERT for about 4 weeks. In some embodiments, the pulsed administration comprises administering complementary ERT for about 6 weeks (i.e., 6 weekly infusions, or 3 infusions every 2 weeks), followed by not administering a complementary ERT for about 2 weeks. In certain embodiments, the pulsed administration comprises administering complementary ERT for about 4 weeks, followed by not administering complementary ERT for about 2 weeks. In some embodiments, the pulsed administration comprises administering complementary ERT for about 2 weeks, followed by not administering complementary ERT for about 2 weeks.

### D. Absence of Long-Term Immune Suppression

In another aspect, the technology relates to methods to treat Pompe disease by administering a AAV expressing a hGAA polypeptide as disclosed herein, where the administration of a composition comprising a AAV-GAA vector is administered to the subject without ongoing immune suppression. That is, in some embodiments, immune suppression is not administered to the subject long term.

In some embodiments, an immune suppressant or immune modulator is administered to the subject intermittently, or for a transient period, e.g., as an immune prophylaxis to the subject to prevent or reduce any immune response to the administered AAV vector, therefore allowing, if necessary, a subsequent or booster administration of the AAV vector expressing GAA according to the methods as disclosed herein.

In some embodiments, an immune modulator is administered for an initial period at, or around the time the AAV vector expressing GAA as disclosed herein is administered to the subject. For example, an immune modulator is administered starting at about 24 hrs before AAV vector expressing GAA is administered to the subject. In some embodiments, an immune modulator is administered starting at about 24 hrs before AAV administration and is administered for at least 1 day, or at least 2 days, or at least 3 days or at least 4 days, or at least 5 days, or at least 6 days, or for about 1 week, or for longer than 1 week after administration of the AAV vector expressing GAA. In some embodiments, an immune modulator is administered starting at, or about 24 hrs before AAV administration and is administered for no more than 1 day, or 2 days, 3 days, or 4 days, or 5 days, or 6 days, or for 1 week, or for 2 weeks, or for 3 weeks or for 1 month after administration of the AAV vector expressing GAA.

In some embodiments, an immune modulator is administered to the subject at tapering lower doses, e.g., at a first dose for a first period of time, at a second lower dose for a second period of time, and third dose that is lower than the second dose—for a third period of time, and so forth until no immune response to the AAV or GAA is produced. For example, in some embodiments, the first dose of an immune modulator is started at, or about 24 hrs before AAV administration and is administered for at least 1 day, or at least 2 days, or at least 3 days or at least 4 days, or at least 5 days, or at least 6 days, or for about 1 week, or about 2 weeks, or about 3 weeks, or about 4 weeks, after which the immune modulator is reduced to a third dose (which is lower than the second dose) for a third period of time (e.g., for at least 1 day, or at least 2 days, or at least 3 days or at least 4 days, or at least 5 days, or at least 6 days, or for about 1 week).

For exemplary purposes only, in some embodiments, the methods to treat Pompe Disease as disclosed herein comprise administering prednisone as an immune suppressant, i.e., immune prophylaxis, at a first dose of 60 milligrams (given orally) starting 24 hours prior to AAV vector administration. In some embodiments, prednisone is continued at 60 mg/day po through the completion of week four after vector administration, after which, at the beginning of week 5 the prednisone dose is tapered to a second dose level of 55 mg/day po and maintained for 7 days. In some embodiments, at the Beginning of week 6 the dose is tapered to a third dose level of 50 mg/day po and maintained for 7 days etc., so that the dose of the immune suppressant (i.e., prednisone) is tapered on a weekly basis by 5 mg/day, after an initial immune suppressant dose for 4 weeks.

The use of prednisone is exemplified herein as an immune suppressant for immune prophylaxis according to the methods as disclosed herein. However, it is envisioned that prednisone can be readily substituted with a different immune modulator and administration regimen known by a person of ordinary skill in the art.

In some embodiments, normal immune prophylaxis for preventing immune reactivity to the expressed GAA is stopped, or withdrawn on day 1, or shortly before or after administration of the rAAV expressing GAA according to the methods as disclosed herein.

### (i) Immune Modulation and Immunosuppression:

As disclosed herein, in some embodiments, the methods to treat Pompe disease by administering a AAV expressing a hGAA polypeptide as disclosed herein to the subject without ongoing immune suppression. That is, in some embodiments, immune suppression is not administered to the subject long term, and is only administered for a short and pre-defined period, including an initial period (with an initial dose) and a tapering period (with incremental tapering doses) after the administration of the AAV vector expressing GAA to the subject. Accordingly, in some embodiments, the immune suppression is administered for between 4 weeks to up to about 15 weeks after the administration of the AAV vector expressing GAA to the subject, and can be administered in an initial and tapering doses as disclosed herein.

Accordingly, in some embodiments, the methods and compositions using the AAV vectors and AAV genomes as described herein, for treating Pompe, further comprises administering an immune modulator for an initial period followed by a tapering period. In some embodiments, the immune modulator can be administered at the time of rAAV vector administration, before rAAV vector administration or, after the rAAV vector administration.

In any embodiment of the methods and compositions as disclosed herein, a subject being administered a rAAV vector or rAAV genome as disclosed herein is also administered an immunosuppressive agent. Various methods are known to result in the immunosuppression of an immune response of a patient being administered AAV. Methods known in the art include administering to the patient an immunosuppressive agent, such as a proteasome inhibitor. One such proteasome inhibitor known in the art, for instance as disclosed in U.S. Pat. No. 9,169,492 and U.S. patent application Ser. No. 15/796,137, both of which are incorporated herein by reference, is bortezomib.

In another embodiment, an immunosuppressive agent can be an antibody, including polyclonal, monoclonal, scfv or other antibody derived molecule that is capable of suppressing the immune response, for instance, through the elimination or suppression of antibody producing cells. In a further embodiment, the immunosuppressive element can be a short hairpin RNA (shRNA). In such an embodiment, the coding region of the shRNA is included in the rAAV cassette and is generally located downstream, 3′ of the poly-A tail. The shRNA can be targeted to reduce or eliminate expression of immunostimulatory agents, such as cytokines, growth factors (including transforming growth factors β1 and β2, TNF and others that are publicly known).

In some embodiments, the immune modulator is an immunoglobulin degrading enzyme such as IdeS, IdeZ, IdeS/Z, Endo S, or, their functional variant. Non-limiting examples of references of such immunoglobulin degrading enzymes and their uses as described in U.S. Pat. Nos. 7,666,582, 8,133,483, US 20180037962, US 20180023070, US 20170209550, U.S. Pat. No. 8,889,128, WO2010/057626, U.S. Pat. Nos. 9,707,279, 8,323,908, US 20190345533, US 20190262434, and WO2020/016318, each of which are incorporated in their entirety by reference.

In some embodiments, the immune modulator or immunosuppressive agent is a proteasome inhibitor. In certain aspects, the proteasome inhibitor is Bortezomib. In some aspects of the embodiment, the immune modulator comprises bortezomib and anti CD20 antibody, Rituximab. In other aspects of the embodiment, the immune modulator comprises bortezomib, Rituximab, methotrexate, and intravenous gamma globulin. Non-limiting examples of such references, disclosing proteasome inhibitors and their combination with Rituximab, methotrexate and intravenous gamma globulin, as described in U.S. Pat. Nos. 10,028,993, 9,592,247, and, U.S. Pat. No. 8,809,282, each of which are incorporated in their entirety by reference. One such proteasome inhibitor known in the art, for instance as disclosed in U.S. Pat. No. 9,169,492 and U.S. patent application Ser. No. 15/796,137, both of which are incorporated herein by reference, is bortezomib.

In another embodiment, an immunosuppressive agent can be an antibody, including polyclonal, monoclonal, scfv or other antibody derived molecule that is capable of suppressing the immune response, for instance, through the elimination or suppression of antibody producing cells. In a further embodiment, the immunosuppressive element can be a short hairpin RNA (shRNA). In such an embodiment, the coding region of the shRNA is included in the rAAV cassette and is generally located downstream, 3′ of the poly-A tail. The shRNA can be targeted to reduce or eliminate expression of immunostimulatory agents, such as cytokines, growth factors (including transforming growth factors β1 and β2, TNF and others that are publicly known).

In alternative embodiments, the immune modulator is an inhibitor of the NF-kB pathway. In certain aspects of the embodiment, the immune modulator is Rapamycin or, a functional variant. Non-limiting examples of references disclosing rapamycin and its use described in U.S. Pat. No. 10,071,114, US 20160067228, US 20160074531, US 20160074532, US 20190076458, U.S. Pat. No. 10,046,064, are incorporated in their entirety. In other aspects of the embodiment, the immune modulator is synthetic nanocarriers comprising an immunosuppressant. Non limiting examples of references of immunosuppresants, immunosuppressants coupled to synthetic nanocarriers, synthetic nanocarriers comprising rapamycin, and/or, toloregenic synthetic nanocarriers, their doses, administration and use as described in US20150320728, US 20180193482, US 20190142974, US 20150328333, US20160243253, U.S. Pat. No. 10,039,822, US 20190076522, US 20160022650, U.S. Pat. Nos. 10,441,651, 10,420,835, US 20150320870, US 2014035636, U.S. Pat. Nos. 10,434,088, 10,335,395, US 20200069659, U.S. Pat. No. 10,357,483, US 20140335186, U.S. Pat. Nos. 10,668,053, 10,357,482, US 20160128986, US 20160128987, US 20200038462, US 20200038463, each of which are incorporated in their entirety by reference.

In some embodiments, the immune modulator is synthetic nanocarriers comprising rapamycin (ImmTOR™ nanoparticles) (Kishimoto, et al., 2016, Nat Nanotechnol, 11(10): 890-899; Maldonado, et al., 2015, PNAS, 112(2): E156-165), as disclosed in US20200038463, U.S. Pat. No. 9,006,254 each of which is incorporated herein in its entirety. In some embodiments, the immune modulator is an engineered cell, e.g., an immune cell that has been modified using SQZ technology as disclosed in WO2017192786, which is incorporated herein in its entirety by reference.

In some embodiments, the immune modulator is selected from the group consisting of poly-ICLC, 1018 ISS, aluminum salts, Amplivax, AS15, BCG, CP-870,893, CpG7909, CyaA, dSLIM, GM-CSF, IC30, IC31, Imiquimod, ImuFact IMP321, IS Patch, ISS, ISCOMATRIX, Juvlmmune, LipoVac, MF59, monophosphoryl lipid A, Montanide IMS 1312, Montanide ISA 206, Montanide ISA 50V, Montanide ISA-51, OK-432, OM-174, OM-197-MP-EC, ONTAK, PEPTEL, vector system, PLGA microparticles, resiquimod, SRL172, Virosomes and other Virus-like particles, YF-17D, VEGF trap, R848, beta-glucan, Pam3Cys, and Aquila's QS21 stimulon. In another further embodiment, the immunomodulator or adjuvant is poly-ICLC.

In some embodiments, the immune modulator is a small molecule that inhibit the innate immune response in cells, such as chloroquine (a TLR signaling inhibitor) and 2-aminopurine (a PKR inhibitor), can also be administered in combination with the composition comprising at least one rAAV as disclosed herein. Some non-limiting examples of commercially available TLR-signaling inhibitors include BX795, chloroquine, CLI-095, OxPAPC, polymyxin B, and rapamycin (all available for purchase from INVIVOGEN™). In addition, inhibitors of pattern recognition receptors (PRR) (which are involved in innate immunity signaling) such as 2-aminopurine, BX795, chloroquine, and H-89, can also be used in the compositions and methods comprising at least one rAAV vector as disclosed herein for in vivo protein expression as disclosed herein.

In some embodiments, a rAAV vector can also encode a negative regulators of innate immunity such as NLRX1. Accordingly, in some embodiments, a rAAV vector can also optionally encode one or more, or any combination of NLRX1, NS1, NS3/4A, or A46R. Additionally, in some embodiments, a composition comprising at least one rAAV vector as disclosed herein can also comprise a synthetic, modified-RNA encoding inhibitors of the innate immune system to avoid the innate immune response generated by the tissue or the subject.

In some embodiments, an immune modulator for use in the administration methods as disclosed herein is an immunosuppressive agent. As used herein, the term “immunosuppressive drug or agent” is intended to include pharmaceutical agents which inhibit or interfere with normal immune function. Examples of immunosuppressive agents suitable with the methods disclosed herein include agents that inhibit T-cell/B-cell costimulation pathways, such as agents that interfere with the coupling of T-cells and B-cells via the CTLA4 and B7 pathways, as disclosed in U.S. Patent Pub. No 2002/0182211. In one embodiment, an immunosuppressive agent is cyclosporine A. Other examples include myophenylate mofetil, rapamicin, and anti-thymocyte globulin. In one embodiment, the immunosuppressive drug is administered in a composition comprising at least one rAAV vector as disclosed herein, or can be administered in a separate composition but simultaneously with, or before or after administration of a composition comprising at least one rAAV vector according to the methods of administration as disclosed herein. An immunosuppressive drug is administered in a formulation which is compatible with the route of administration and is administered to a subject at a dosage sufficient to achieve the desired therapeutic effect. In some embodiments, the immunosuppressive drug is administered transiently for a sufficient time to induce tolerance to the rAAV vector as disclosed herein.

Various methods are known to result in the immunosuppression of an immune response of a patient being administered AAV. Methods known in the art include administering to the patient an immunosuppressive agent, such as a proteasome inhibitor. One such proteasome inhibitor known in the art, for instance as disclosed in U.S. Pat. No. 9,169,492 and U.S. patent application Ser. No. 15/796,137, both of which are incorporated herein by reference, is bortezomib. In some embodiments, an immunosuppressive agent can be an antibody, including polyclonal, monoclonal, scfv or other antibody derived molecule that is capable of suppressing the immune response, for instance, through the elimination or suppression of antibody producing cells. In a further embodiment, the immunosuppressive element can be a short hairpin RNA (shRNA). In such an embodiment, the coding region of the shRNA is included in the rAAV cassette and is generally located downstream, 3′ of the poly-A tail. The shRNA can be targeted to reduce or eliminate expression of immunostimulatory agents, such as cytokines, growth factors (including transforming growth factors β1 and β2, TNF and others that are publicly known).

The use of such immune modulating agents facilitates the ability to for one to use multiple dosing (e.g., multiple administration) over numerous months and/or years. This permits using multiple agents as discussed below, e.g., a rAAV vector encoding multiple genes, or multiple administrations to the subject.

In some aspects of the invention, the recombinant AAV comprising a nucleic acid encoding human GAA is produced by the triple transfection method that uses close ended linear duplexed DNA molecules that lack bacterial backbone sequences, for example, as described in PCT/US2021/013689, published as WO/2021/146591, which is incorporated herein by reference in its entirety. In some embodiments, the rAAV of the invention is manufactured using plasmid DNA as starting material. In several embodiments, the rAAV of the invention is manufactured using close ended linear duplexed DNA as starting material. Non-limiting examples of close ended linear duplex nucleic acids include doggy bone DNA (dbDNA) or dumbbell-shaped DNA. The close ended linear duplex nucleic acids may be generated within cells or using in vitro cell free system. Cell free in vitro synthesis of dumbbell-shaped DNA and doggy bone DNA are described in U.S. Pat. No. 6,451,563; Efficient production of superior dumbbell-shaped DNA minimal vectors for small hairpin RNA expression-Nucleic Acids Res. 2015 Oct. 15; 43(18): e120; High-Purity Preparation of a Large DNA Dumbbell-Antisense & nucleic acid drug development 11:149-153 (2001); U.S. Pat. Nos. 9,109,250; 9,499,847; 10,501,782; and WO 2018033730 A1; all of which are herein incorporated by reference in their entireties. DNA from cell free in vitro synthesis is devoid of any prokaryotic DNA modifications (e.g., is substantially free of bacterial DNA).

One example of an in vitro process for producing a closed linear DNA (e.g., containing the ITRs described herein) comprises (a) contacting a DNA template flanked on either side by a protelomerase target sequence with at least one DNA polymerase in the presence of one or more primers under conditions promoting amplification of said template; and (b) contacting amplified DNA produced in (a) with at least one protelomerase under conditions promoting formation of a closed linear expression cassette DNA. The closed linear DNA may be a closed DNA expression cassette DNA product that may comprise, consist or consist essentially of a eukaryotic promoter operably linked to a coding sequence of interest and optionally, a eukaryotic transcription termination sequence. The closed linear expression cassette DNA product may additionally lack one or more bacterial or vector sequences, typically selected from the group consisting of: (i) bacterial origins of replication; (ii) bacterial selection markers (typically antibiotic resistance genes) and (iii) unmethylated CpG motifs.

### E. Pharmaceutical Compositions

The rAAV vectors as disclosed herein for use in the methods of administration as disclosed herein can be formulated in a pharmaceutical composition with a pharmaceutically acceptable excipient, i.e., one or more pharmaceutically acceptable carrier substances and/or additives, e.g., buffers, carriers, excipients, stabilizers, etc. The pharmaceutical composition may be provided in the form of a kit. Pharmaceutical compositions comprising the rAAV vectors as disclosed herein for use in the methods of administration as disclosed herein and uses thereof are known in the art.

Accordingly, a further aspect of the invention provides a pharmaceutical composition comprising a rAAV vector as disclosed herein for use in the methods of administration as disclosed herein. Relative amounts of the active ingredient (e.g., a rAAV vectors aa disclosed herein), a pharmaceutically acceptable excipient, and/or any additional ingredients in a pharmaceutical composition in accordance with the present disclosure may vary, depending upon the identity, size, and/or condition of the subject being treated and further depending upon the route by which the composition is to be administered. For example, the composition may comprise between 0.1 percent and 99 percent (w/w) of the active ingredient. By way of example, the composition may comprise between 0.1 percent and 100 percent, e.g., between 0.5 and 50 percent, between 1-30 percent, between 5-80 percent, at least 80 percent (w/w) active ingredient.

The pharmaceutical compositions can be formulated using one or more excipients or diluents to (1) increase stability; (2) increase cell transfection or transduction; (3) permit the sustained or delayed release of the payload; (4) alter the biodistribution (e.g., target the viral particle to specific tissues or cell types); (5) increase the translation of encoded protein; (6) alter the release profile of encoded protein and/or (7) allow for regulatable expression of the payload of the invention. In some embodiments, a pharmaceutically acceptable excipient may be at least 95 percent, at least 96 percent, at least 97 percent, at least 98 percent, at least 99 percent, or 100 percent pure. In some embodiments, an excipient is approved for use for humans and for veterinary use. In some embodiments, an excipient may be approved by United States Food and Drug Administration. In some embodiments, an excipient may be of pharmaceutical grade. In some embodiments, an excipient may meet the standards of the United States Pharmacopoeia (USP), the European Pharmacopoeia (EP), the British Pharmacopoeia, and/or the International Pharmacopoeia. Excipients, as used herein, include, but are not limited to, any and all solvents, dispersion media, diluents, or other liquid vehicles, dispersion or suspension aids, surface active agents, isotonic agents, thickening or emulsifying agents, preservatives, and the like, as suited to the particular dosage form desired. Various excipients for formulating pharmaceutical compositions and techniques for preparing the composition are known in the art (see Remington: The Science and Practice of Pharmacy, 21 st Edition, A. R. Gennaro, Lippincott, Williams and Wilkins, Baltimore, MD, 2006; incorporated herein by reference in its entirety). The use of a conventional excipient medium may be contemplated within the scope of the present disclosure, except insofar as any conventional excipient medium may be incompatible with a substance or its derivatives, such as by producing any undesirable biological effect or otherwise interacting in a deleterious manner with any other component(s) of the pharmaceutical composition.

### Compositions/Formulations

The rAAV vectors as disclosed herein can be formulated in a composition. For example, the rAAV vectors as disclosed herein can be formulated in a pharmaceutical composition with a pharmaceutically acceptable excipient, i.e., one or more pharmaceutically acceptable carrier substances and/or additives, e.g., buffers, carriers, excipients, stabilisers, etc. The composition, e.g., the pharmaceutical composition may be provided in the form of a kit. It is noted the terms “composition” and “formulation” are used interchangeably here.

Accordingly, in one aspect, provided herein is a composition comprising the recombinant AAV vector particles described herein. Generally, the composition comprises the recombinant AAV vector particles described herein at a concentration from about 1e⁹ vg/ml to about 1e¹⁵ vg/ml. In some embodiments, the composition comprises the recombinant AAV vector particles described herein at a concentration from about 1e¹⁰ vg/ml to about 1e¹⁴ vg/ml. In some embodiments, the composition comprises the recombinant AAV vector particles described herein at a concentration from about 1e¹² vg/ml to about 1e¹⁰ vg/ml. In some embodiments, the composition comprises the recombinant AAV vector particles described herein at a concentration from about 1e¹² vg/ml to about 1e¹⁵ vg/ml. For example, the composition comprises the recombinant AAV vector particles described herein at a concentration from about 3e⁹ vg/ml to about 3e¹³ vg/ml, from about 2.5e¹⁰ vg/ml to about 1e⁴ vg/ml, from about 3e¹⁰ vg/ml to about 1e¹³ vg/ml, or from 1e¹¹ vg/ml to about 5e¹² vg/ml.

In some embodiments, the composition comprises the recombinant AAV vector particles described herein at a concentration of about 1e¹¹ vg/ml, or about 1.5e¹² vg/ml, or about 2e¹¹ vg/ml, or about 2.5e¹² vg/ml, or about 3e¹² vg/ml, or about 3.5e¹² vg/ml, or about 4e¹² vg/ml, or about 4.5e¹² vg/ml, or about 5e¹² vg/ml, or about 5.5e¹² vg/ml, or about 6e¹² vg/ml, or about 6.5e¹² vg/ml, or about 7e¹² vg/ml, or about 7.5e¹² vg/ml, or about 8e¹² vg/ml, or about 8.5e¹² vg/ml, or about 9e¹² vg/ml, or about 9.5e¹³ vg/ml, or about 1e¹³ vg/ml, or about 1.5e¹³ vg/ml, or about 2e¹³ vg/ml, or about 2.5e¹³ vg/ml, or about 3e¹³ vg/ml, or about 3.5e¹³ vg/ml, or about 4e¹³ vg/ml, or about 4.5e¹³ vg/ml, or about 5e¹³ vg/ml, or about 5.5e¹³ vg/ml, or about 6e¹³ vg/ml, or about 6.5e¹³ vg/ml, or about 7e¹³ vg/ml, or about 7.5e¹³ vg/ml, or about 8e¹³ vg/ml, or about 8.5e¹³ vg/ml, or about 9e¹³ vg/ml, or about 9.5e¹³ vg/ml, or about 1e¹4 vg/ml.

The pharmaceutical composition comprises the population of purified recombinant adeno-associated virus (rAAV) described herein. The pharmaceutical composition comprising the rAAV, comprises a buffer of pH about 6.5 to about 8.0. In some embodiments, the pH is about 6.5 to about 7.5. For example, the pH is from about 6.5, about 6.6, about 6.7, about 6.8, about 6.9, about 7.0, about 7.1, about 7.2, about 7.3, about 7.4 or about 7.5. In some preferred embodiments, the pH is less than about 7.5. For example, the pH is less than about 7.4, less than about 7.3, less than about 7.2, less than about 7.1, less than about 7.0, less than about 6.9, less than about 6.8, less than about 6.7, or less than about 6.6. In some embodiments, the pharmaceutical composition comprises one or, more excipients, comprising one or, more multivalent ions and/or, salts thereof. In some embodiments, the multivalent ions can be selected or, optionally selected from the group consisting of citrate, sulfate, magnesium and phosphate. In some embodiments, the pharmaceutical composition comprises one or, more excipients, comprising one or, more ions selected or, optionally selected from the group consisting of, sodium, potassium, chloride, ammonium, carbonate, nitrate, chlorate, chlorite, and calcium. In some embodiments, the pharmaceutical composition comprising the rAAV, further comprises a non-ionic surfactant. In some embodiments, the non-ionic surfactant is selected from the group consisting of polyoxyethylene fatty alcohol ethers, polyoxyethylene alkyl phenyl ethers, polyoxyethylene-polyoxypropylene block copolymers, alkylglucosides, alkyl phenol ethoxylates, preferably polysorbates, polyoxyethylene alkyl phenyl ethers, and any combinations thereof. In some embodiments, non-ionic surfactant is selected from the group consisting of TWEEN 60 nonionic detergent, PPG-PEG-PPG Pluronic 10R5, Polyoxyethylene (18) tridecyl ether, Polyoxyethylene (12) tridecyl ether, MERPOL SH surfactant, MERPOL OJ surfactant, MERPOL HCS surfactant, Poloxamer P188, Poloxamer P407, Poloxamer P338 IGEPAL CO-720, IGEPAL CO-630, IGEPAL CA-720, Brij S20, BrijS10, Brij 010, Brij C10, BRIJ 020, ECOSURF EH-9,ECOSURF EH-14, TERGITOL 15-S-7, PF-68, ECOSURF SA-15, TERGITOL15-S-9, TERGITOL 15-S-12, TERGITOL L-64, TERGITOLNP-7, TERGITOL NP-8, TERGITOL NP-9, TERGITOL NP-9.5, TERGITOL NP-10, TERGITOL NP-11, TERGITOL NP-12, TERGITOLNP-13, polysorbate 20, and any combinations thereof. In some embodiments, the pharmaceutical composition further comprises polyol, or, sugar, or similar. See, e.g., International Patent No. WO2022/159679, which is incorporated herein by reference in its entirety.

In some embodiments, the composition comprises a buffer. It is noted that any physiological buffer can be used. Non-limiting examples of buffers include, but are not limited to, PBS, Tris.HCl, phosphate, citric acid, histidine, tromethamine, succinic acid, malic acid, α-ketoglutaric acid, carbonate (bicarbonate-carbonic acid buffer), and protein buffers. In some embodiments, the buffer is PBS. In some embodiments, the buffer comprises Tris. In some embodiments, buffer is Tris.HCl. In some embodiments, the buffer is histidine buffer.

Generally, the buffer has a salt concentration of from about 50 mM to about 750 mM. For example, the buffer has a salt concentration from about 75 mM to about 700 mM, from about 100 mM to about 650 mM, from about 120 mM to about 600 mM, or from about 140 mM to about 550 mM. In some embodiments, the buffer has a salt concentration from about 150 mM to about 400 mM. In some embodiments, the buffer has a salt concentration of about 150 mM, about 175 mM, about 200 mM, about 225 mM, about 250 mM, about 275 mM, about 300 mM, about 325 mM, about 350 mM, about 375 mM, about 400 mM, about 425 mM, about 450 mM, or about 475 mM. In some preferred embodiments, the buffer has a salt concentration of about 150 mM, about 200 mM or about 365 mM.

In some embodiments, the ionic strength of the composition is at least about 100 mM. For example, the ionic strength of the composition is from about 125 mM to about 750 mM, or from about 150 mM to about 500 mM, or from about 175 mM to about 700 mM, from about 200 mM to about 600 mM, or from about 225 mM to about 550 mM, or from about 250 mM to about 500 mM, or from about 275 mM to about 450 mM, or from about 300 mM to about 400 mM. In some embodiments, the ionic strength of the composition is at least about 125 mM, at least about 150 mM, at least about 175 mM, at least about 200 mM, at least about 225 mM, at least about 250 mM, at least about 275 mM, at least about 300 mM, at least about 325 mM, at least about 350 mM, at least about 375 mM, at least about 400 mM, at least about 425 mM, at least about 450 mM, at least about 475 mM or at least about 500 mM. In some embodiments, the ionic strength of the composition is less than 100 mM, for example about 95 mM, about 90 mM, about 85 mM, about 80 mM, about 75 mM, about 70 mM, about 65 mM, about 60 mM, about 55 mM, about 50 mM, or, even less.

The osmolarity of the composition is maintained at near isotonic levels. For example, the osmolarity of the composition can be from about 100 mOsm to about 600 mOsm, such as from about 125 mOsm to about 500 mOsm, or, from about 130 mOsm to about 350 mOsm, or, from about 140 mOsm to about 400 mOsm, or, from about 140 mOsm to about 350 mOsm, or from about 200 mOsm to about 400 mOsm, or from about 500 mOsm to about 600 mOsm, or from about 200 mOsm to about 600 mOsm, or from about 300 mOsm to about 600 mOsm, or from about 200 mOsm to about 500 mOsm, or from about 300 mOsm to about 400 mOsm, or from about 150 mOsm to about 350 mOsm, or from about 175 mOsm to about 300 mOsm, or from about 300 mOsm to about 375 mOsm, or from about 200 mOsm to about 350 mOsm, or from about 225 mOsm to about 325 mOs, or from about 525 mOsm to about 590 mOsm. In some embodiments, the composition comprises an isotonic solution.

Generally, the composition has a pH of about 6.5 to about 8.0. For example, the composition has a pH of about 6.5 to about 7.5. In some embodiments, the composition has a pH of from about 7 to about 8. For example, the composition has a pH of from about 7.3 to about 7.9. In some other non-limiting example, the composition has a pH of from about 7.4 to about 7.8 or from about 7.4 to about 7.7. In some embodiments, the composition has a pH of from about 7.3 to about 7.6, e.g., from about 7.3 to about 7.55. In some preferred embodiments, the composition has a pH less than about 7.5. For example, the composition has a pH about 7.4 or lower, about 7.3 or lower, about 7.2 or lower, about 7.1 or lower, about 7.0 or lower, about 6.9 or lower, about 6.8 or lower, about 6.7 or lower, about 6.6 or lower, or about 6.5 or lower.

The composition can comprise one or more ions and/or salts thereof. Exemplary ions include, but are not limited to sodium, potassium, chloride, magnesium ammonium, carbonate, nitrate, chlorate, chlorite, and calcium. The ions can be provided as a salt, such as a halide (F, Cl, Br, I) salt of sodium, potassium, magnesium, and/or calcium, non-limiting examples of which include NaCl, KCl, MgCl₂, CaCl₂, and combinations thereof. Additional exemplary salts that can be used include, but are not limited to, carboxylic acid salts, such as acetates, propionates, pyrrol idonecarboxylates (or pidolates) or sorbates; poly hydroxylated carboxylic acid salts, such as gluconates, heptagluconates, ketogluconates, lactate gluconates, ascorbates or pantothenates; mono- or polycarboxyl hydroxy acid salts, such as citrates or lactates; amino acid salts, such as aspartates or glutamates; and fulvate salts. The salts are individually included at a concentration of from about 500 μM to about 500 mM.

In some embodiments, the composition comprises one or more multivalent ions and/or salts thereof. Exemplary multivalent ions include, but are not limited to, calcium, citrate, sulfate, magnesium, and phosphate. Multivalent ions and/or salts thereof can be individually included in the composition at a concentration of from about 500 μM to about 500 mM, for example, at a concentration of about 500 μM, about 750 μM, about 1 mM, about 1.3 mM, about 1.5 mM, about 1.7 mM, about 2.3 mM, about 2.5 mM, about 2.7 mM, about 3.3 mM, about 3.5 mM, about 3.7 mM, about 4.3 mM, about 4.5 mM, about 4.7 mM, about 5 mM, about 10 mM, about 25 mM, about 50 mM, about 75 mM, about 80 mM, about 85 mM, about 90 mM, about 95 mM, about 100 mM, about 125 mM, about 150 mM, about 175 mM, about 200 mM, about 225 mM, about 250 mM, about 275 mM, about 300 mM, about 325 mM, about 350 mM, about 375 mM, about 400 mM, about 425 mM, about 450 mM, about 475 mM, or about 500 mM. Non limiting examples of salts are NaCl, KCl, CaCl₂, CaSO₄, MgSO₄, Na₃PO₄, CaCO₃, NaNO₃, Al₂(SO4)₃.

In some embodiments, the composition comprises NaCl. When present, NaCl can be at a concentration from about 100 mM to about 500 mM, or from about 125 mM to about 450 mM, or from about 100 mM to about 200 mM, or from about 150 mM to about 200 mM. For example, the composition can comprise NaCl at a concentration from about 150 mM to about 425 mM, from about 175 mM to about 400 mM, or from about 175 mM to about 375 mM, or from about 200 mM to about 375 mM.

In some embodiments, the composition comprises KCl. When present, KCl can be at a concentration from about 1 mM to about 10 mM. For example, the composition can comprise KCl at a concentration from about 1.5 mM to about 7.5 mM.

In some embodiments, the composition comprises CaCl₂. When present, CaCl₂ can be at a concentration from about 0.1 mM to about 2 mM. For example, the composition can comprise CaCl₂ at a concentration from about 0.5 mM to about 1.5 mM. In some embodiments, the composition comprises CaCl₂ at a concentration from about 0.75 mM to about 1.25 mM.

In some embodiments, the composition comprises MgCl₂. When present, MgCl₂ can be at a concentration from about 0.1 mM to about 1.5 mM. For example, the composition can comprise MgCl₂ at a concentration from about 0.25 mM to about 1 mM or from about 0.25 mM to about 0.75 mM.

In some embodiments, the composition comprises MgSO₄. When present, MgSO₄ can be at a concentration from about 5 mM to about 150 mM. For example, the composition can comprise MgSO₄ at a concentration from about 10 mM to about 120 mM, or from about 10 mM to about 50 mM, or from about 15 mM to about 45 mM, or about 75 mM to about 125 mM, or from about 80 mM to about 100 mM, or from about 85 mM to about 95 mM, or from about 15 mM to about 100 mM.

In some embodiments, the composition comprises phosphate, e.g., mono basic or dibasic phosphate or a salt thereof. When present, the phosphate, e.g., mono basic or dibasic phosphate or a salt thereof can be at a concentration from about 5 mM to about 30 mM. For example, the composition can comprise phosphate, e.g., mono basic or dibasic phosphate or a salt thereof at a concentration from about 7.5 mM to about 25 mM. In some embodiments, the composition comprises phosphate, e.g., mono basic or dibasic phosphate or a salt thereof at a concentration from about 10 mM to about 20 mM.

In some embodiments, the composition comprises a mono basic phosphate or a salt thereof at a concentration from about 0.25 mM to about 3 mM. For example, the composition comprises a mono basic phosphate or a salt thereof at a concentration from about 0.5 mM to about 2.75 mM, or from about 0.75 mM to about 2.5 mM or from about 1 mM to about 2.25 mM. In some embodiments, the mono basic phosphate or salt thereof is potassium phosphate monobasic.

In some embodiments, the composition comprises a dibasic phosphate or a salt thereof at a concentration from about 5 mM to about 15 mM. For example, the composition comprises a dibasic phosphate or a salt thereof at a concentration from about 7.5 mM to about 12.5 mM or from about 8 mM to about 10 mM. In some embodiments, the dibasic phosphate or a salt thereof is sodium phosphate dibasic. In some embodiments, the composition is substantially free of dibasic phosphate, e.g., sodium phosphate dibasic.

In some embodiments, the composition comprises Tris (e.g., Tris.HCl) or a salt thereof at a concentration from about 1 mM to about 50 mM. For example, the composition comprises Tris (e.g., Tris.HCl) or a salt thereof at a concentration of from about 5 mM to about 40 mM, or from about 7.5 mM to about 35 mM, or from about 10 mM to about 30 mM or from about 15 mM to about 25 mM.

In some embodiments, the composition comprises histidine or a salt thereof at a concentration from about 1 mM to about 50 mM. For example, the composition comprises histidine or a salt thereof at a concentration of from about 5 mM to about 40 mM, or from about 7.5 mM to about 35 mM, or from about 10 mM to about 30 mM or from about 15 mM to about 25 mM.

The composition can also comprise a bulking agent. Exemplary bulking agents include, but are not limited to sugars, polyols and (PVP K24). Exemplary polyols include, but are not limited to, polyhydroxy hydrocarbons, monosaccharides, disaccharides, and trisaccharides. Some exemplary polyols include but are not limited to, sorbitol, mannitol, glycerol, propylene glycol, polyethylene glycol, dulcitol, sucrose, lactose, maltose, trehalose and dextran. In some embodiments, polyol is sorbitol, sucrose or mannitol. In some embodiments, the bulking agent is sorbitol. In some embodiments, the bulking agent is sucrose. In some embodiments, the bulking agent is mannitol. In some embodiments, the bulking agent is trehalose, e.g., trehalose dehydrate. In some embodiments, the bulking agent is a dextran, e.g., Dextran T40 and/or Dextran T10.

When present, the bulking agent can be present at a concentration of from about 0.5% (w/v) to about 10% (w/v). For example, the composition can comprise a bulking agent, e.g., a polyol or providone (PVP K24) at a concentration from about from about 1% (w/v) to about 7.5% (w/v), e.g., from about 1% (w/v) to about 4% (w/v) or from about 4% (w/v) to about 6% (w/v).

In some embodiments, the composition comprises glycerol, sorbitol, sucrose, or mannitol at a concentration from about 1% (w/v) to about 10% (w/v). In some embodiments, the composition comprises glycerol, sorbitol, sucrose, or mannitol at a concentration from about 1% (w/v) to about 10% (w/v). In some embodiments, the composition comprises sorbitol at concentration from about 3% (w/v) to about 6% (w/v). In some embodiments, the composition comprises sorbitol at concentration of about 1% (w/v), about 2% (w/v), about 3% (w/v), about 4% (w/v), about 5% (w/v), about 6% (w/v), about 7% (w/v), about 8% (w/v), about 9% (w/v), or about 10% (w/v). In some embodiments, the composition comprises sucrose at concentration from about 3% (w/v) to about 6% (w/v). In some embodiments, the composition comprises sucrose at concentration of about 1% (w/v), about 2% (w/v), about 3% (w/v), about 4% (w/v), about 5% (w/v), about 6% (w/v), about 7% (w/v), about 8% (w/v), about 9% (w/v), or about 10% (w/v). In some embodiments, the composition comprises mannitol at concentration from about 3% (w/v) to about 6% (w/v). In some embodiments, the composition comprises mannitol at concentration of about 1% (w/v), about 2% (w/v), about 3% (w/v), about 4% (w/v), about 5% (w/v), about 6% (w/v), about 7% (w/v), about 8% (w/v), about 9% (w/v), or about 10% (w/v).

The composition can also comprise a non-ionic surfactant. The non-ionic surfactant can be selected from the group consisting of polyoxyethylene fatty alcohol ethers, polyoxyethylene alkyl phenyl ethers, polyoxyethylene-polyoxypropylene block copolymers, alkylglucosides, alkyl phenol ethoxylates, preferably polysorbates, polyoxyethylene alkyl phenyl ethers, and any combinations thereof. Non-limiting examples of suitable non-ionic surfactants include polyoxyethylene (12) isooctylphenyl ether (e.g., IGEPAL® CA-270 polyoxyethylene (12) isooctylphenyl ether), polyoxyethylenesorbitan monooleate (e.g., TWEEN® 80 polyoxyethylenesorbitan monooleate), polyethylene glycol octadecyl ether (e.g., Brij® S20 polyethylene glycol octadecyl ether), seed oil surfactant (e.g., Ecosurf™ SA-15 seed oil surfactant), poloxamer 188 (a copolymer of polyoxyethylene and polyoxypropylene), nonylphenol ethoxylate (e.g., Tergitol™ NP-10 nonylphenol ethoxylate), and combinations thereof. In some embodiments, the non-ionic surfactant is selected from the group consisting of TWEEN 60 nonionic detergent, PPG-PEG-PPG Pluronic 10R5, Pluronic F-68 (PF 68), Polyoxyethylene (18) tridecyl ether, Polyoxyethylene (12) tridecyl ether, MERPOL SH surfactant, MERPOL OJ surfactant, MERPOL HCS surfactant, Poloxamer P188, Poloxamer P407, Poloxamer P 338, IGEPAL CO-720, IGEPAL CO-630, IGEPAL CA-720, Brij S20, BrijS10, Brij 010, Brij C10, BRIJ 020, ECOSURF EH-9,ECOSURF EH-14, TERGITOL 15-S-7, ECOSURF SA-15, TERGITOL15-S-9, TERGITOL 15-S-12, TERGITOL L-64, TERGITOLNP-7, TERGITOL NP-8, TERGITOL NP-9, TERGITOL NP-9.5, TERGITOL NP-10, TERGITOL NP-11, TERGITOL NP-12, TERGITOLNP-13, polysorbate 20, and any combinations thereof. In some embodiments, the non-ionic surfactant is Poloxamer P 188, Poloxamer P407, Pluronic F-68, Ecosurf SA-15, Brij S20, Tergitol NP-10, IGEPAL CA 720 or Tween 80. In some embodiments, the composition is substantially free of a non-ionic surfactant. In some embodiments, the non-ionic surfactant is not a polysorbate, e.g., Tween 80 (also referred to as polysorbate 80 or PS80).

When present, the non-ionic surfactant can be present at a concentration from about 0.0001% (w/v) to about 0.01% (w/v). For example, the composition can comprise a non-ionic surfactant at a concentration from about 0.0005% (w/v) to about 0.0015% (w/v). In some embodiments, the composition can comprise a non-ionic surfactant at a concentration of about 0.0001% (w/v), about 0.0002% (w/v), about 0.0003% (w/v), about 0.0004% (w/v), about 0.0005% (w/v), about 0.0006% (w/v), about 0.0007% (w/v), about 0.0008% (w/v), about 0.0009% (w/v), about 0.001% (w/v), about 0.002% (w/v), about 0.003% (w/v), about 0.004% (w/v), about 0.005% (w/v), about 0.006% (w/v), about 0.007% (w/v), about 0.008% (w/v), about 0.009% (w/v), or about 0.01%. (w/v). In some preferred embodiments, the composition comprises a non-ionic surfactant at a concentration of about 0.0005% (w/v) or about 0.001% (w/v).

In some embodiments, the composition comprises, in addition to the rAAV, a buffer (e.g., PBS, Tris.HCl, phosphate, citric acid, histidine, tromethamine, succinic acid, malic acid, α-ketoglutaric acid, carbonate buffer), a bulking agent (e.g., a polyol such as sorbitol, mannitol, glycerol, propylene glycol, polyethylene glycol, dulcitol, sucrose, lactose, maltose, trehalose and dextran) and a non-ionic surfactant (e.g., Poloxamer P 188, Poloxamer P407, Pluronic F-68, Ecosurf SA-15, Brij S20, Tergitol NP-10, IGEPAL CA 720 or Tween 80).

In some embodiments, the composition comprises, in addition to the rAAV, a buffer (e.g., PBS, Tris.HCl, phosphate, citric acid, histidine, tromethamine, succinic acid, malic acid, α-ketoglutaric acid, carbonate buffer), a bulking agent (e.g., a polyol such as sorbitol, mannitol, glycerol, propylene glycol, polyethylene glycol, dulcitol, sucrose, lactose, maltose, trehalose and dextran), a non-ionic surfactant (e.g., Poloxamer P 188, Poloxamer P407, Pluronic F-68, Ecosurf SA-15, Brij S20, Tergitol NP-10, IGEPAL CA 720 or Tween 80), and a multivalent ion (e.g., a multivalent ion selected from the group consisting of calcium, citrate, sulfate, and magnesium).

In some embodiments, the composition comprises, in addition to the rAAV, a buffer (e.g., PBS, Tris.HCl, phosphate, citric acid, histidine, tromethamine, succinic acid, malic acid, α-ketoglutaric acid, carbonate buffer), a bulking agent (e.g., a polyol such as sorbitol, mannitol, glycerol, propylene glycol, polyethylene glycol, dulcitol, sucrose, lactose, maltose, trehalose and dextran), and a multivalent ion (e.g., a multivalent ion selected from the group consisting of calcium, citrate, sulfate, and magnesium).

It is noted that any one of the specific buffers or group of buffers listed in the description of the compositions can be used with any one of the specific bulking agents or group of bulking agents listed in the description of the compositions and with any of the specific non-ionic surfactants or group of surfactants listed in the description of the compositions and with any of the specific multivalent ions and multivalent ion group listed in the description of the compositions. Similarly, any one of the specific bulking agents or group of bulking agents listed in the description of the compositions can be used with any one of the specific buffers or group of buffers listed in the description of the compositions and with any of the specific non-ionic surfactants or group of surfactants listed in the description of the compositions and with any of the specific multivalent ions and multivalent ion group listed in the description of the compositions. Likewise, any of the specific non-ionic surfactants or group of surfactants listed in the description of the compositions can be used with any one of the specific buffers or group of buffers listed in the description of the compositions and with any one of the specific bulking agents or group of bulking agents listed in the description of the compositions and with any of the specific multivalent ions and multivalent ion group listed in the description of the compositions. As well, any of the specific multivalent ions and multivalent ion group listed in the description of the compositions can be used with any one of the specific buffers or group of buffers listed in the description of the compositions and with any one of the specific bulking agents or group of bulking agents listed in the description of the compositions and with any of the specific non-ionic surfactants or group of surfactants listed in the description of the compositions. In other words, all individual specific combinations of buffers, buffer group, bulking agents, bulking agent groups, non-ionic surfactants, non-ionic surfactant groups, multivalent ions and multivalent ion groups listed in the description of the compositions are specifically contemplated and claimed.

### Exemplary Compositions

In some embodiments, the composition, e.g., the pharmaceutical composition comprises, in addition to the rAAV, about 10 mM Phosphate pH 7.4, about 200 mM NaCl, about 5 mM KCl, about 1% (w/v) mannitol, and about 0.0005% (w/v) IGEPAL CA 720.

In some embodiments, the composition, e.g., the pharmaceutical composition comprises, in addition to the rAAV, about 20 mM Phosphate pH 7.4, about 300 mM NaCl, about 3 mM KCl, about 3% (w/v) mannitol, and about 0.001% (w/v) Brij 520.

In some embodiments, the composition, e.g., the pharmaceutical composition comprises, in addition to the rAAV, about 20 mM Phosphate pH 7.4, about 300 mM NaCl, about 3 mM KCl, about 3% (w/v) sorbitol, and about 0.001% (w/v) Ecosurf SA-15.

In some embodiments, the composition, e.g., the pharmaceutical composition comprises, in addition to the rAAV, about 10 mM Phosphate pH 7.4, about 350 mM NaCl, about 2.7 mM KCl, about 5% (w/v) sorbitol, and about 0.001% (w/v) poloxamer 188.

In some embodiments, the composition, e.g., the pharmaceutical composition comprises, in addition to the rAAV, about 10 mM Phosphate pH 6.95-7.2, about 137 mM NaCl, about 2.7 mM KCl, about 0.9 mM CaCl₂, about 0.5 mM MgCl₂, and about 0.001% (w/v) Pluronic F-68.

In some embodiments, the composition, e.g., the pharmaceutical composition comprises, in addition to the rAAV, about 10 mM Phosphate pH 7.3, about 180 mM NaCl, about 2.7 mM KCl, about 5% (w/v) sorbitol, and about 0.001% (w/v) Poloxamer 188.

In some embodiments, the composition, e.g., the pharmaceutical composition comprises, in addition to the rAAV, about 15 mM Phosphate pH 7.4, about 375 mM NaCl, about 3.5 mM KCl, about 5% (w/v) sorbitol, and about 0.0005% (w/v) Tergitol NP-10.

In some embodiments, the c composition, e.g., the pharmaceutical composition comprises, in addition to the rAAV, about 15 mM Phosphate pH 7.4, about 375 mM NaCl, about 3.5 mM KCl, about 3% (w/v) glycerol, and about 0.0005% (w/v) Tween 80.

In some embodiments, the composition, e.g., the pharmaceutical composition comprises, in addition to the rAAV, about 10 mM Phosphate pH 7.6, about 137 mM NaCl, about 2.7 mM KCl, about 5% (w/v) sorbitol, and about 0.01% Pluronic F-68.

In some embodiments, the composition, e.g., the pharmaceutical composition comprises, in addition to the rAAV, about 10 mM Phosphate pH 7.4, about 137 mM NaCl, about 2.7 mM KCl, about 5% (w/v) sorbitol, about 0.01% Pluronic F-68, and about 20 mM MgSO₄.

In some embodiments, the composition, e.g., the pharmaceutical composition comprises, in addition to the rAAV, about 10 mM Phosphate pH 7.6, about 137 mM NaCl, about 2.7 mM KCl, about 5% (w/v) mannitol, and about 0.01% Pluronic F-68.

In some embodiments, the composition, e.g., the pharmaceutical composition comprises, in addition to the rAAV, about 10 mM Phosphate pH 7.3, about 137 mM NaCl, about 2.7 mM KCl, about 5% (w/v) mannitol, about 0.01% Pluronic F-68, and about 20 mM MgSO₄.

In some embodiments, the composition, e.g., the pharmaceutical composition comprises, in addition to the rAAV, about 10 mM Phosphate pH 7.4, about 137 mM NaCl, about 2.7 mM KCl, about 5% (w/v) sorbitol, and about 20 mM MgSO₄.

In some embodiments, the composition, e.g., the pharmaceutical composition comprises, in addition to the rAAV, about 10 mM Phosphate pH 7.4, about 137 mM NaCl, about 2.7 mM KCl, about 5% (w/v) mannitol, and about 20 mM MgSO₄.

In some embodiments, the composition, e.g., the pharmaceutical composition comprises recombinant AAV vector (rAAV), in 10 mM Phosphate pH 7.4, 200 mM NaCl, 5 mM KCl, 1% (w/v) mannitol, 0.0005% (w/v) IGEPAL CA 720 to a fill volume of 5 ml. In some embodiments, the fill volume is 1 ml, 2 ml, 3 ml, 4 ml, 5 ml, 6 ml, 7 ml, 8 ml, 9 ml, or, 10 ml.

In some embodiments, the composition, e.g., the pharmaceutical composition comprises recombinant AAV vector (rAAV), in 20 mM Phosphate pH 7.4, 300 mM NaCl, 3 mM KCl, 3% (w/v) mannitol, 0.001% (w/v) Brij S20 to a fill volume of 5 ml. In some embodiments, the fill volume is 1 ml, 2 ml, 3 ml, 4 ml, 5 ml, 6 ml, 7 ml, 8 ml, 9 ml, or, 10 ml.

In some embodiments, the composition, e.g., the pharmaceutical composition comprises recombinant AAV vector (rAAV), in 20 mM Phosphate pH 7.4, 300 mM NaCl, 3 mM KCl, 3% (w/v) sorbitol, 0.001% (w/v) Ecosurf SA-15 to a fill volume of 5 ml. In some embodiments, the fill volume is 1 ml, 2 ml, 3 ml, 4 ml, 5 ml, 6 ml, 7 ml, 8 ml, 9 ml, or, 10 ml.

In some embodiments, the composition, e.g., the pharmaceutical composition comprises recombinant AAV vector (rAAV), in 10 mM Phosphate pH 7.4, 350 mM NaCl, 2.7 mM KCl, 5% (w/v) sorbitol, 0.001% (w/v) poloxamer 188 to a fill volume of 5 ml. In some embodiments, the fill volume is 1 ml, 2 ml, 3 ml, 4 ml, 5 ml, 6 ml, 7 ml, 8 ml, 9 ml, or, 10 ml.

In some embodiments, the composition, e.g., the pharmaceutical composition comprises recombinant AAV vector (rAAV), in 15 mM Phosphate pH 7.4, 375 mM NaCl, 3.5 mM KCl, 5% (w/v) sorbitol, 0.0005% (w/v) Tergitol NP-10 to a fill volume of 5 ml. In some embodiments, the fill volume is 1 ml, 2 ml, 3 ml, 4 ml, 5 ml, 6 ml, 7 ml, 8 ml, 9 ml, or, 10 ml.

In some embodiments, the composition, e.g., the pharmaceutical composition comprises recombinant AAV vector (rAAV), in 15 mM Phosphate pH 7.4, 375 mM NaCl, 3.5 mM KCl, 3% (w/v) glycerol, 0.0005% (w/v) Tween 80 to a fill volume of 5 ml. In some embodiments, the fill volume is 1 ml, 2 ml, 3 ml, 4 ml, 5 ml, 6 ml, 7 ml, 8 ml, 9 ml, or, 10 ml.

Additional exemplary compositions/compositions comprising rAAV are described in PCT/US2022/0137279, the content of which is incorporated herein by reference in its entirety.

All compositions stored between −60° C. to about −80° C.

The rAAV vectors as disclosed herein for use in the methods of administration as disclosed herein may be used in combination with one or more other therapeutic, prophylactic, research or diagnostic agents. By “in combination with,” it is not intended to imply that the agents must be administered at the same time and/or formulated for delivery together, although these methods of delivery are within the scope of the present invention. Compositions can be administered concurrently with, prior to, or subsequent to, one or more other desired therapeutics or medical procedures. In some embodiments, the delivery of one treatment (e.g., gene therapy vectors) is still occurring when the delivery of the second (e.g., one or more therapeutic) begins, so that there is overlap in terms of administration. This is sometimes referred to herein as “simultaneous” or “concurrent delivery.” In other embodiments, the delivery of one treatment ends before the delivery of the other treatment begins. In some embodiments of either case, the treatment is more effective because of combined administration. For example, the second treatment is more effective, e.g., an equivalent effect is seen with less of the second treatment, or the second treatment reduces symptoms to a greater extent, than would be seen if the second treatment were administered in the absence of the first treatment, or the analogous situation is seen with the first treatment. In some embodiments, delivery is such that the reduction in a symptom, or other parameter related to the disorder is greater than what would be observed with one treatment delivered in the absence of the other. The effect of the two treatments can be partially additive, wholly additive, or greater than additive. The delivery can be such that an effect of the first treatment delivered is still detectable when the second is delivered. The composition described herein and the at least one additional therapy can be administered simultaneously, in the same or in separate compositions, or sequentially. For sequential administration, the gene therapy vectors described herein can be administered first, and the one or more therapeutic can be administered second, or the order of administration can be reversed. The gene therapy vectors and the one or more therapeutic can be administered during periods of active disorder, or during a period of remission or less active disease. The gene therapy vectors can be administered before another treatment, concurrently with the treatment, post-treatment, or during remission of the disorder.

When administered in combination, the rAAV vectors as disclosed herein for use in the methods of administration as disclosed herein and the one or more therapeutic (e.g., second or third therapeutic), or all, can be administered in an amount or dose that is higher, lower or the same as the amount or dosage of each used individually, e.g., as a monotherapy. In certain embodiments, the administered amount or dosage of a rAAV vector as disclosed herein for use in the methods of administration as disclosed herein and the one or more therapeutic (e.g., second or third agent), or all, is lower (e.g., at least 20%, at least 30%, at least 40%, or at least 50%) than the amount or dosage of each used individually. In other embodiments, the amount or dosage of the rAAV vector as disclosed herein for use in the methods of administration as disclosed herein and the one or more therapeutic (e.g., second or third agent), or all, that results in a desired effect (e.g., treatment of a cardiovascular disease or heart disease) is lower (e.g., at least 20%, at least 30%, at least 40%, or at least 50% lower) than the amount or dosage of each individually required to achieve the same therapeutic effect.

In some embodiments, the methods of administration of a rAAV vector as disclosed herein can deliver a rAVV vector disclosed herein alone, or in combination with an additional agent, for example, an immune modulator as disclosed herein.

In some embodiments, the AAV vectors expressing GAA as disclosed herein are not administered concurrently with, or in combination with ERT. In alternative embodiments, the AAV vectors expressing GAA as disclosed herein are administered in combination with ERT for a maximum period of 24 weeks or shorter than 24 weeks after administration of the AAV expressing ERT. In some embodiments, the AAV vectors expressing GAA as disclosed herein are administered in combination with an immune modulator for an initial period and, optionally a tapering period after administration of the AAV expressing ERT.

### VI. Definitions

The following terms are used in the description herein and the appended claims:

The terms “a,” “an,” “the” and similar references used in the context of describing the present invention (especially in the context of the following claims) are to be construed to cover both the singular and the plural, unless otherwise indicated herein or clearly contradicted by context. Further, ordinal indicators—such as “first,” “second,” “third,” etc.—for identified elements are used to distinguish between the elements, and do not indicate or imply a required or limited number of such elements, and do not indicate a particular position or order of such elements unless otherwise specifically stated. All methods described herein can be performed in any suitable order unless otherwise indicated herein or otherwise clearly contradicted by context. The use of any and all examples, or exemplary language (e.g., “such as”) provided herein is intended merely to better illuminate the present invention and does not pose a limitation on the scope of the invention otherwise claimed. No language in the present specification should be construed as indicating any non-claimed element essential to the practice of the invention.

Furthermore, the term “about,” as used herein when referring to a measurable value such as an amount of the length of a polynucleotide or polypeptide sequence, dose, time, temperature, and the like, is meant to encompass variations of ±20%, ±10%, ±5%, ±1%, ±0.5%, or even ±0.1% of the specified amount.

Also as used herein, “and/or” refers to and encompasses any and all possible combinations of one or more of the associated listed items, as well as the lack of combinations when interpreted in the alternative (“or”).

As used herein, the transitional phrase “consisting essentially of” means that the scope of a claim is to be interpreted to encompass the specified materials or steps recited in the claim, “and those that do not materially affect the basic and novel characteristic(s)” of the claimed invention. See, In re Herz, 537 F.2d 549, 551-52, 190 USPQ 461,463 (CCPA 1976) (emphasis in the original); see also MPEP § 2111.03. Thus, the term “consisting essentially of” when used in a claim of this invention is not intended to be interpreted to be equivalent to “comprising.” Unless the context indicates otherwise, it is specifically intended that the various features of the invention described herein can be used in any combination.

Moreover, the present invention also contemplates that in some embodiments of the invention, any feature or combination of features set forth herein can be excluded or omitted.

To illustrate further, if, for example, the specification indicates that a particular amino acid can be selected from A, G, I, Land/or V, this language also indicates that the amino acid can be selected from any subset of these amino acid(s) for example A, G, I or L; A, G, I or V; A or G; only L; etc. as if each such subcombination is expressly set forth herein. Moreover, such language also indicates that one or more of the specified amino acids can be disclaimed (e.g., by negative proviso). For example, in particular embodiments the amino acid is not A, G or I; is not A; is not G or V; etc. as if each such possible disclaimer is expressly set forth herein.

The term “parvovirus” as used herein encompasses the family Parvoviridae, including autonomously replicating parvoviruses and dependoviruses. The autonomous parvoviruses include members of the genera Parvovirus, Erythrovirus, Densovirus, Iteravirus, and Contravirus. Exemplary autonomous parvoviruses include, but are not limited to, minute virus of mouse, bovine parvovirus, canine parvovirus, chicken parvovirus, feline panleukopenia virus, feline parvovirus, goose parvovirus, H1 parvovirus, Muscovy duck parvovirus, B19 virus, and any other autonomous parvovirus now known or later discovered. Other autonomous parvoviruses are known to those skilled in the art. See, e.g., BERNARD N. FIELDS et al., VIROLOGY, volume 2, chapter 69 (4th ed., Lippincott-Raven Publishers).

As used herein, the term “adeno-associated virus” (AAV), includes but is not limited to, AAV type 1, AAV type 2, AAV type 3 (including types 3A and 3B), AAV type 4, AAV type 5, AAV type 6, AAV type 7, AAV type 8, AAV type 9, AAV type 10, AAV type 11, avian AAV, bovine AAV, canine AAV, equine AAV, ovine AAV, and any other AAV now known or later discovered. See, e.g., BERNARD N. FIELDS et al., VIROLOGY, volume 2, chapter 69 (4th ed., Lippincott-Raven Publishers). A number of relatively new AAV serotypes and clades have been identified (see, e.g., Gao et al., (2004) J. Virology 78:6381-6388; Moris et al., (2004) Virology 33-:375-383); and also Table 1 as disclosed in U.S. Provisional Application 62,937,556, filed on Nov. 19, 2019 and Table 1 in International Applications WO2020/102645, and WO2020/102667, each of which is incorporated herein in their entirety.

The genomic sequences of various serotypes of AAV and the autonomous parvoviruses, as well as the sequences of the native inverted terminal repeats (ITRs), Rep proteins, and capsid subunits are known in the art. Such sequences may be found in the literature or in public databases such as GenBank. See, e.g., GenBank Accession Numbers NC 002077, NC_001401, NC_001729, NC_001863, NC 001829, NC 001862, NC 000883, NC_001701, NC 001510, NC_006152, NC_006261, AF063497, U89790, AF043303, AF028705, AF028704, J02275, J01901, J02275, X01457, AF288061, AH009962, AY028226, AY028223, NC 001358, NC_001540, AF513851, AF513852, AY530579; the disclosures of which are incorporated by reference herein for teaching parvovirus and AAV nucleic acid and amino acid sequences. See also, e.g., Srivistava et al., (1983) J Virology 45:555; Chiarini et al., (1998) J. Virology 71:6823; Chiarini et al., (1999) J. Virology 73:1309; Bantel-Schaal et al., (1999) J. Virology 73:939; Xiao et al., (1999) J. Virology 73:3994; Muramatsu et al., (1996) Virology 221:208; Shade et al., (1986) J. Viral. 58:921; Gao et al., (2002) Proc. Nat. Acad. Sci. USA 99:11854; Morris et al., (2004) Virology 33-:375-383; international patent publications WO 00/28061, WO 99/61601, WO 98/11244; and U.S. Pat. No. 6,156,303; the disclosures of which are incorporated by reference herein for teaching parvovirus and AAV nucleic acid and amino acid sequences. See also Table 1 and Table 5 disclosed in U.S. Pat. No. 62,937,556, filed on Nov. 19, 2019 or Table 1 as disclosed in International Applications WO2020/102645, and WO2020/102667, each of which is incorporated herein in their entirety. The capsid structures of autonomous parvoviruses and AAV are described in more detail in BERNARD N. FIELDS et al., VIROLOGY, volume 2, chapters 69 & 70 (4th ed., Lippincott-Raven Publishers). See also, description of the crystal structure of AAV2 (Xie et al., (2002) Proc. Nat. Acad. Sci. 99:10405-10), AAV4 (Padron et al., (2005) J. Viral. 79: 5047-58), AAV5 (Walters et al., (2004) J. Viral. 78: 3361-71) and CPV (Xie et al., (1996) J. Mal. Bio. 6:497-520 and Tsao et al., (1991) Science 251: 1456-64).

The term “tropism” as used herein refers to preferential entry of the virus into certain cells or tissues, optionally followed by expression (e.g., transcription and, optionally, translation) of a sequence(s) carried by the viral genome in the cell, e.g., for a recombinant virus, expression of a heterologous nucleic acid(s) of interest.

As used here, “systemic tropism” and “systemic transduction” (and equivalent terms) indicate that the virus capsid or virus vector of the invention exhibits tropism for and/or transduces tissues throughout the body (e.g., brain, lung, skeletal muscle, heart, liver, kidney and/or pancreas).

As used herein, “selective tropism” or “specific tropism” means delivery of virus vectors to and/or specific transduction of certain target cells and/or certain tissues.

Unless indicated otherwise, “efficient transduction” or “efficient tropism,” or similar terms, can be determined by reference to a suitable control (e.g., at least about 50%, 60%, 70%, 80%, 85%, 90%, 95%, 100%, 125%, 150%, 175%, 200%, 250%, 300%, 350%, 400%, 500% or more of the transduction or tropism, respectively, of the control). In particular embodiments, the virus vector efficiently transduces or has efficient tropism for liver cells and muscle cells. Suitable controls will depend on a variety of factors including the desired tropism and/or transduction profile.

Similarly, it can be determined if a virus “does not efficiently transduce” or “does not have efficient tropism” for a target tissue, or similar terms, by reference to a suitable control. In particular embodiments, the virus vector does not efficiently transduce (i.e., has does not have efficient tropism) for kidney, gonads and/or germ cells. In particular embodiments, transduction (e.g., undesirable transduction) of tissue(s) (e.g., kidney) is 20% or less, 10% or less, 5% or less, 1% or less, 0.1% or less of the level of transduction of the desired target tissue(s) (e.g., liver, skeletal muscle, diaphragm muscle, cardiac muscle and/or cells of the central nervous system).

As used herein, the term “polypeptide” encompasses both peptides and proteins, unless indicated otherwise.

A “polynucleotide” is a sequence of nucleotide bases, and may be RNA, DNA or DNA-RNA hybrid sequences (including both naturally occurring and non-naturally occurring nucleotides), but in representative embodiments are either single or double stranded DNA sequences.

The terms “heterologous nucleotide sequence” and “heterologous nucleic acid molecule” are used interchangeably herein and refer to a nucleic acid sequence that is not naturally occurring in the virus. Generally, the heterologous nucleic acid molecule or heterologous nucleotide sequence comprises an open reading frame that encodes a polypeptide and/or nontranslated RNA of interest (e.g., for delivery to a cell and/or subject).

A “chimeric nucleic acid” comprises two or more nucleic acid sequences covalently linked together to encode a fusion polypeptide. The nucleic acids may be DNA, RNA, or a hybrid thereof.

The term “fusion polypeptide” comprises two or more polypeptides covalently linked together, typically by peptide bonding.

As used herein, an “isolated” polynucleotide (e.g., an “isolated DNA” or an “isolated RNA”) means a polynucleotide at least partially separated from at least some of the other components of the naturally occurring organism or virus, for example; the cell or viral structural components or other polypeptides or nucleic acids commonly found associated with the polynucleotide. In representative embodiments an “isolated” nucleotide is enriched by at least about 10-fold, 100′-fold, 1000-fold, 10,000-fold or more as compared with the starting material.

Likewise, an “isolated” polypeptide means a polypeptide that is at least partially separated from at least some of the other components of the naturally occurring organism or virus, for example, the cell or viral structural components or other polypeptides or nucleic acids commonly found associated with the polypeptide. In representative embodiments an “isolated” polypeptide is enriched by at least about 10-fold, 100-fold, 1000-fold, 10,000-fold or more as compared with the starting material.

An “isolated cell” refers to a cell that is separated from other components with which it is normally associated in its natural state. For example, an isolated cell can be a cell in culture medium and/or a cell in a pharmaceutically acceptable carrier of this invention. Thus, an isolated cell can be delivered to and/or introduced into a subject. In some embodiments, an isolated cell can be a cell that is removed from a subject and manipulated as described herein ex vivo and then returned to the subject.

A population of virions can be generated by any of the methods described herein. In one embodiment, the population is at least 101 virions. In one embodiment, the population is at least 102 virions, at least 103, virions, at least 104 virions, at least 105 virions, at least 106 virions, at least 107 virions, at least 108 virions, at least 109 virions, at least 1010 virions, at least 1011 virions, at least 1012 virions, at least 1013 virions, at least 1014 virions, at least 1015 virions, at least 1016 virions, or at least 1017 virions. A population of virions can be heterogeneous or can be homogeneous (e.g., substantially homogeneous or completely homogeneous).

A “substantially homogeneous population” as the term is used herein, refers to a population of virions that are mostly identical, with few to no contaminant virions (those that are not identical) therein. A substantially homogeneous population is at least 90% of identical virions (e.g., the desired virion), and can be at least 91%, at least 92%, at least 93%, at least 94%, at least 95%, at least 96%, at least 97%, at least 98%, at least 99%, at least 99.5%, at least 99.9% of identical virions.

A population of virions that is completely homogeneous contains only identical virions.

As used herein, by “isolate” or “purify” (or grammatical equivalents) a virus vector or virus particle or population of virus particles, it is meant that the virus vector or virus particle or population of virus particles is at least partially separated from at least some of the other components in the starting material. In representative embodiments an “isolated” or “purified” virus vector or virus particle or population of virus particles is enriched by at least about 10-fold, 100-fold, 1000-fold, 10,000-fold or more as compared with the starting material.

Unless indicated otherwise, “efficient transduction” or “efficient tropism,” or similar terms, can be determined by reference to a suitable control (e.g., at least about 10%, 15%, 20%, 25%, 30%, 35%, 40%, 45%, 50%, 55%, 60%, 65%, 70%, 75%, 80%, 85%, 90%, 95%, 100%, 125%, 150%, 175%, 200%, 250%, 300%, 350%, 400%, 500% or more of the transduction or tropism, respectively, of the control). In particular embodiments, the virus vector efficiently transduces or has efficient tropism for neuronal cells and cardiomyocytes. Suitable controls will depend on a variety of factors including the desired tropism and/or transduction profile.

A “therapeutic polypeptide” is a polypeptide that can alleviate, reduce, prevent, delay and/or stabilize symptoms that result from an absence or defect in a protein in a cell or subject and/or is a polypeptide that otherwise confers a benefit to a subject, e.g., enzyme replacement to reduce or eliminate symptoms of a disease, or improvement in transplant survivability or induction of an immune response.

The terms “heterologous nucleotide sequence” and “heterologous nucleic acid molecule” are used interchangeably herein and refer to a nucleic acid sequence that is not naturally occurring in the virus. Generally, the heterologous nucleic acid molecule or heterologous nucleotide sequence comprises an open reading frame that encodes a polypeptide and/or nontranslated RNA of interest (e.g., for delivery to a cell and/or subject), for example the GAA polypeptide.

As used herein, the terms “virus vector,” “vector” or “gene delivery vector” refer to a virus (e.g., AAV) particle that functions as a nucleic acid delivery vehicle, and which comprises the vector genome (e.g., viral DNA [vDNA]) packaged within a virion. Alternatively, in some contexts, the term “vector” may be used to refer to the vector genome/vDNA alone.

An “rAAV vector genome” or “rAAV genome” is an AAV genome (i.e., vDNA) that comprises one or more heterologous nucleic acid sequences. rAAV vectors generally require only the inverted terminal repeat(s) (TR(s)) in cis to generate virus. All other viral sequences are dispensable and may be supplied in trans (Muzyczka, (1992) Curr. Topics Microbial. Immunol. 158:97). Typically, the rAAV vector genome will only retain the one or more TR sequence so as to maximize the size of the transgene that can be efficiently packaged by the vector. The structural and non-structural protein coding sequences may be provided in trans (e.g., from a vector, such as a plasmid, or by stably integrating the sequences into a packaging cell). In embodiments of the invention the rAAV vector genome comprises at least one ITR sequence (e.g., AAV TR sequence), optionally two ITRs (e.g., two AAV TRs), which typically will be at the 5′ and 3′ ends of the vector genome and flank the heterologous nucleic acid, but need not be contiguous thereto. The TRs can be the same or different from each other.

The term “terminal repeat” or “TR” includes any viral terminal repeat or synthetic sequence that forms a hairpin structure and functions as an inverted terminal repeat (i.e., an ITR that mediates the desired functions such as replication, virus packaging, integration and/or provirus rescue, and the like). The TR can be an AAV TR or a non-AAV TR. For example, a non-AAV TR sequence such as those of other parvoviruses (e.g., canine parvovirus (CPV), mouse parvovirus (MVM), human parvovirus B-19) or any other suitable virus sequence (e.g., the SV40 hairpin that serves as the origin of SV40 replication) can be used as a TR, which can further be modified by truncation, substitution, deletion, insertion and/or addition. Further, the TR can be partially or completely synthetic, such as the “double-D sequence” as described in U.S. Pat. No. 5,478,745 to Samulski et al.

An “AAV terminal repeat” or “AAV TR,” including an “AAV inverted terminal repeat” or “AAV ITR” may be from any AAV, including but not limited to serotypes 1, 2, 3, 4, 5, 6, 7, 8, 9, 10, 11 or 12 or any other AAV now known or later discovered. An AAV terminal repeat need not have the native terminal repeat sequence (e.g., a native AAV TR or AAV ITR sequence may be altered by insertion, deletion, truncation and/or missense mutations), as long as the terminal repeat mediates the desired functions, e.g., replication, virus packaging, integration, and/or provirus rescue, and the like.

AAV proteins VP1, VP2 and VP3 are capsid proteins that interact together to form an AAV capsid of an icosahedral symmetry. VP1.5 is an AAV capsid protein described in US Publication No. 2014/0037585.

The virus vectors of the invention can further be “targeted” virus vectors (e.g., having a directed tropism) and/or a “hybrid” parvovirus (i.e., in which the viral TRs and viral capsid are from different parvoviruses) as described in international patent publication WO 00/28004 and Chao et al., (2000) Molecular Therapy 2:619.

The virus vectors of the invention can further be duplexed parvovirus particles as described in international patent publication WO 01/92551 (the disclosure of which is incorporated herein by reference in its entirety). Thus, in some embodiments, double stranded (duplex) genomes can be packaged into the virus capsids of the invention.

Further, the viral capsid or genomic elements can contain other modifications, including insertions, deletions and/or substitutions.

A “chimeric’ capsid protein as used herein means an AAV capsid protein (e.g., any one or more of VP1, VP2 or VP3) that has been modified by substitutions in one or more (e.g., 2, 3, 4, 5, 6, 7, 8, 9, 10, etc.) amino acid residues in the amino acid sequence of the capsid protein relative to wild type, as well as insertions and/or deletions of one or more (e.g., 2, 3, 4, 5, 6, 7, 8, 9, 10, etc.) amino acid residues in the amino acid sequence relative to wild type. In some embodiments, complete or partial domains, functional regions, epitopes, etc., from one AAV serotype can replace the corresponding wild type domain, functional region, epitope, etc. of a different AAV serotype, in any combination, to produce a chimeric capsid protein of this invention. Production of a chimeric capsid protein can be carried out according to protocols well known in the art and a significant number of chimeric capsid proteins are described in the literature as well as herein that can be included in the capsid of this invention.

As used herein, the term “haploid AAV” shall mean that AAV as described in International Application WO2018/170310, or US Application US2018/037149, which are incorporated herein in their entirety by reference. In some embodiments, a population of virions is a haploid AAV population where a virion particle can be constructed wherein at least one viral protein from the group consisting of AAV capsid proteins, VP1, VP2 and VP3, is different from at least one of the other viral proteins, required to form the virion particle capable of encapsulating an AAV genome. For each viral protein present (VP1, VP2, and/or VP3), that protein is the same type (e.g., all AAV2 VP1). In one instance, at least one of the viral proteins is a chimeric viral protein and at least one of the other two viral proteins is not a chimeric. In one embodiment VP1 and VP2 are chimeric and only VP3 is non-chimeric. For example, only the viral particle composed of VP1/VP2 from the chimeric AAV2/8 (the N-terminus of AAV2 and the C-terminus of AAV8) paired with only VP3 from AAV2; or only the chimeric VP1/VP2 28m-2P3 (the N-terminal from AAV8 and the C-terminal from AAV2 without mutation of VP3 start codon) paired with only VP3 from AAV2. In another embodiment only VP3 is chimeric and VP1 and VP2 are non-chimeric. In another embodiment at least one of the viral proteins is from a completely different serotype. For example, only the chimeric VP1/VP2 28m-2P3 paired with VP3 from only AAV3. In another example, no chimeric is present.

The term a “hybrid” AAV vector or parvovirus refers to a rAAV vector where the viral TRs or ITRs and viral capsid are from different parvoviruses. Hybrid vectors are described in international patent publication WO 00/28004 and Chao et al., (2000) Molecular Therapy 2:619. For example, a hybrid AAV vector typically comprises the adenovirus 5′ and 3′ cis ITR sequences sufficient for adenovirus replication and packaging (i.e., the adenovirus terminal repeats and PAC sequence).

The term “polyploid AAV” refers to a AAV vector which is composed of capsids from two or more AAV serotypes, e.g., and can take advantages from individual serotypes for higher transduction but not in certain embodiments eliminate the tropism from the parents.

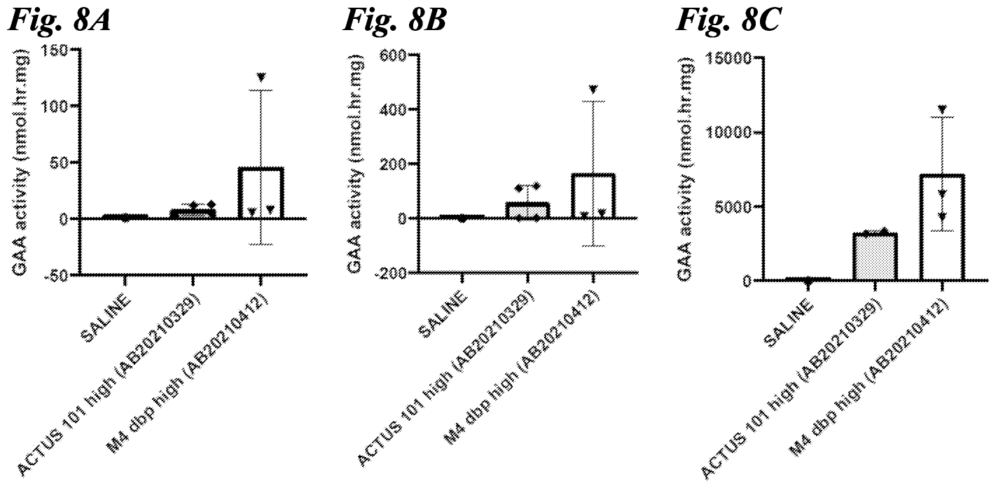

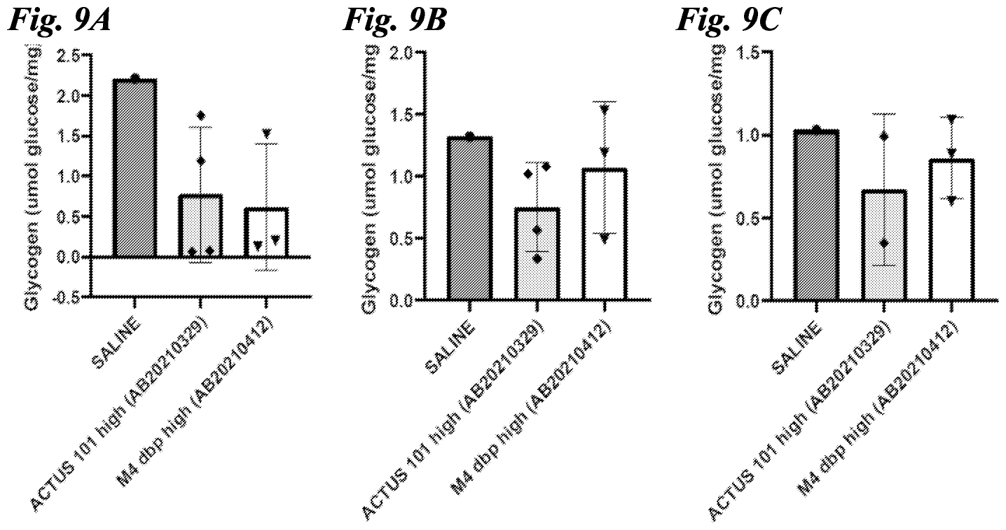

The term “GAA” or “GAA polypeptide,” as used herein, encompasses mature (˜76 or ˜67 kDa) and precursor (e.g., ˜110 kDa) GAA as well as modified (e.g., truncated or mutated by insertion(s), deletion(s) and/or substitution(s)) GAA proteins or fragments thereof that retain biological function (i.e., have at least one biological activity of the native GAA protein, e.g., can hydrolyze glycogen, as defined above) and GAA variants (e.g., GAA II as described by Kunita et al., (1997) Biochemica et Biophysica Acta 1362:269; GAA polymorphisms and SNPs are described by Hirschhorn, R. and Reuser, A. J. (2001) in The Metabolic and Molecular Basis for Inherited Disease (Scriver, C. R., Beaudet. A. L., Sly, W. S. & Valle, D. Eds.), pp. 3389-3419, McGraw-Hill, New York, see pages 3403-3405; each incorporated herein by reference in its entirety). Any GAA coding sequence known in the art may be used, for example, see the coding sequences of FIGS. 8 and 9; GenBank Accession number NM_00152 and Hoefsloot et al., (1988) EMBO J. 7:1697 and Van Hove et al., (1996) Proc. Natl. Acad. Sci. USA 93:65 (human), GenBank Accession number NM_008064 (mouse), and Kunita et al., (1997) Biochemica et Biophysics Acta 1362:269 (quail); the disclosures of which are incorporated herein by reference for their teachings of GAA coding and noncoding sequences. The term “GAA”, or, “hGAA” or, “rhGAA” is used interchangeably to refer to nucleic acid encoding human GAA or, to human GAA polypeptide.

The term “targeting peptide” is also referred to as a “targeting sequence” as used herein is intended to refer to a peptide that targets a particular subcellular compartment, for example, a mammalian lysosome. A targeting peptide encompassed for use herein is a lysosome targeting peptide that is mannose-6-phosphate-independent. An exemplary targeting sequence is an IGF2 targeting peptide as disclosed herein.

The term “signal sequence” is used interchangeably herein with the term “secretory signal sequence” or “leader sequence” or “signal peptide” or variations thereof, and intended to refer to amino acid sequences that function to enhance (as defined above) secretion of an operably linked polypeptide, (e.g., a GAA peptide) from the cell as compared with the level of secretion seen with the native polypeptide. As defined above, by “enhanced” secretion, it is meant that the relative proportion of GAA polypeptide synthesized by the cell that is secreted from the cell is increased; it is not necessary that the absolute amount of secreted protein is also increased. In particular embodiments of the invention, essentially all (i.e., at least 95%, 97%, 98%, 99% or more) of the GAA-polypeptide is secreted. It is not necessary, however, that essentially all or even most of the GAA polypeptide is secreted, as long as the level of secretion is enhanced as compared with the native GAA polypeptide. Exemplary leader sequences include, but are not limited to the innate GAA signal sequence (also referred to as endogenous GAA signal sequence), AAT sequence, IL2(1-3), IL2 leader sequence (IL2 wt), a modified IL2 leader sequence (IL2 mut), fibronectin (FN1, also referred to as FBN), or IgG leader sequence or functional variants thereof, as disclosed herein.

As used herein, the term “amino acid” encompasses any naturally occurring amino acid, modified forms thereof, and synthetic amino acids. Naturally occurring, levorotatory (L-) amino acids are disclosed in Table 2 of US Publication 2018/0371496, which is incorporated herein in its entirety. Alternatively, the amino acid can be a modified amino acid residue (nonlimiting examples are shown in Table 4 of US Publication of US Publication 2018/0371496) and/or can be an amino acid that is modified by post-translation modification (e.g., acetylation, amidation, formylation, hydroxylation, methylation, phosphorylation or sulfatation). Further, the non-naturally occurring amino acid can be an “unnatural” amino acid as described by Wang et al., Annu Rev Biophys Biomol Struct. 35:225-49 (2006). These unnatural amino acids can advantageously be used to chemically link molecules of interest to the AAV capsid protein.

To illustrate further, if, for example, the specification indicates that a particular amino acid can be selected from A, G, I, L and/or V, this language also indicates that the amino acid can be selected from any subset of these amino acid(s) for example A, G, I or L; A, G, I or V; A or G; only L; etc. as if each such subcombination is expressly set forth herein. Moreover, such language also indicates that one or more of the specified amino acids can be disclaimed (e.g., by negative proviso). For example, in particular embodiments the amino acid is not A, G or I; is not A; is not G or V; etc. as if each such possible disclaimer is expressly set forth herein.

As used herein, the phrase “promoter” refers to a region of DNA that generally is located upstream of a nucleic acid sequence to be transcribed that is needed for transcription to occur, i.e. which initiates transcription. Promoters permit the proper activation or repression of transcription of a coding sequence under their control. A promoter typically contains specific sequences that are recognized and bound by plurality of TFs. TFs bind to the promoter sequences and result in the recruitment of RNA polymerase, an enzyme that synthesizes RNA from the coding region of the gene. A great many promoters are known in the art.

The term “synthetic promoter” as used herein relates to a promoter that does not occur in nature. Parts of the synthetic promoter may be naturally occurring (e.g. the minimal promoter), but the synthetic promoter as a complete entity is not naturally occurring.

As used herein, “minimal promoter” (also known as the “core promoter”) refers to a short DNA segment which is inactive or largely inactive by itself, but can mediate transcription when combined with other transcription regulatory elements. Minimum promoter sequence can be derived from various different sources, including prokaryotic and eukaryotic genes. Examples of minimal promoters are discussed above, and include the dopamine beta-hydroxylase gene minimum promoter, cytomegalovirus (CMV) immediate early gene minimum promoter (CMV-MP), and the herpes thymidine kinase minimal promoter (MinTK). A minimal promoter typically comprises the transcription start site (TSS) and elements directly upstream, a binding site for RNA polymerase II, and general transcription factor binding sites (often a TATA box).

As used herein, “proximal promoter” relates to the minimal promoter plus the proximal sequence upstream of the gene that tends to contain primary regulatory elements. It often extends approximately 250 base pairs upstream of the TSS, and includes specific TFBS. The proximal promoter can be a naturally occurring liver-specific proximal promoter. However, the proximal promoter can be synthetic.

A “functional variant” of a promoter or other nucleic acid sequence in the context of the present invention is a variant of a reference sequence that retains the ability to function in the same way as the reference sequence, e.g. as a liver-specific promoter. Alternative terms for such functional variants include “biological equivalents” or “equivalents”.

The terms “liver-specific” or “liver-specific expression” when in reference to a promoter refers to the ability of promoter to enhance or drive expression of a gene in the liver (or in liver-derived cells) in a preferential or predominant manner as compared to other tissues (e.g. spleen, muscle, heart, lung, and brain). Expression of the gene can be in the form of mRNA or protein. In some embodiments, liver-specific expression is such that there is negligible expression in other (i.e. non-liver) tissues or cells, i.e. expression is highly liver-specific. In some embodiments, while a liver-specific promoter drives expression preferentially in the liver, it can also drive expression of the gene in another tissue of interest at a lower level, e.g., muscle.

The skilled person can thus easily determine whether any variant of the liver-specific promoter recited above remains functional (i.e. it is a functional variant as defined above). For example, any given promoter to be assessed can be operably linked to a minimal promoter (e.g. positioned upstream of CMV-MP) and the ability of the promoter to drive liver-specific expression of a gene (typically a reporter gene) is measured. Similarly, the ability of a promoter to drive liver-specific expression can be readily assessed by the skilled person (e.g. as described in the examples below). Expression levels of a gene driven by a variant of a reference promoter can be compared to the expression levels driven by the reference sequence. In some embodiments, where liver-specific expression levels driven by a variant promoter are at least 50%, at least 60%, at least 70%, at least 80%, at least 90%, or at least 100% of the expression levels driven by the reference promoter, it can be said that the variant remains functional. Suitable nucleic acid constructs and reporter assays to assess liver-specific expression enhancement can easily be constructed, and the examples set out below give suitable methodologies.

Liver-specificity can be identified wherein the expression of a gene (e.g. a therapeutic or reporter gene) occurs preferentially or predominantly in liver-derived cells. Preferential or predominant expression can be defined, for example, where the level of expression is significantly greater in liver-derived cells than in other types of cells (i.e. non-liver-derived cells). For example, expression in liver-derived cells is suitably at least 5-fold higher than non-liver cells, preferably at least 10-fold higher than non-liver cells, and it may be 50-fold higher or more in some cases. For convenience, liver-specific expression can suitably be demonstrated via a comparison of expression levels in a hepatic cell line (e.g. liver-derived cell line such as Huh7 and/or HepG2 cells) or liver primary cells, compared with expression levels in a kidney-derived cell line (e.g. HEK-293), a cervical tissue-derived cell line (e.g. HeLa) and/or a lung-derived cell line (e.g. A549).

The synthetic liver-specific promoters of the present invention are preferably suitable for promoting expression in the liver of a subject, e.g., driving liver-specific expression of a transgene, preferably a therapeutic transgene.

Preferred synthetic liver-specific promoters of the present invention are suitable for promoting liver-specific transgene expression and have an activity in liver cells which is at least 15%, 20%, 30%, 40%, 50%, 60%, 70%, 80%, 90%, 100%, 125%, 150%, 175%, 200%, 250%, 300%, 350% or 400% of the activity of the TBG promoter (see, e.g., SEQ ID NO: 435 as disclosed in International Application WO2021102107).

The synthetic liver-specific promoters of the present invention are preferably suitable for promoting liver-specific expression at a level at least 1.5-fold greater than a CMV-IE promoter (see, e.g., SEQ ID NO: 433 as disclosed in International Application WO2021102107) in liver-derived cells, preferably at least 2-fold greater than a CMV promoter in liver-derived cells (e.g. HEK-293, HeLa, and/or A549 cells).

The terms “identity” and “identical” and the like refer to the sequence similarity between two polymeric molecules, e.g., between two nucleic acid molecules, such as between two DNA molecules. Sequence alignments and determination of sequence identity can be done, e.g., using the Basic Local Alignment Search Tool (BLAST) originally described by Altschul et al. 1990 (J Mol Biol 215: 403-10), such as the “Blast 2 sequences” algorithm described by Tatusova and Madden 1999 (FEMS Microbiol Lett 174: 247-250).

The term “synthetic” as used herein means a nucleic acid molecule that does not occur in nature. Synthetic nucleic acid expression constructs of the present invention are produced artificially, typically by recombinant technologies. Such synthetic nucleic acids may contain naturally occurring sequences (e.g. promoter, enhancer, intron, and other such regulatory sequences), but these are present in a non-naturally occurring context. For example, a synthetic gene (or portion of a gene) typically contains one or more nucleic acid sequences that are not contiguous in nature (chimeric sequences), and/or may encompass substitutions, insertions, and deletions and combinations thereof.

A “spacer sequence” or “spacer” as used herein is a nucleic acid sequence that separates two functional nucleic acid sequences. It can have essentially any sequence, provided it does not prevent the functional nucleic acid sequence (e.g. cis-regulatory element) from functioning as desired (e.g. this could happen if it includes a silencer sequence, prevents binding of the desired transcription factor, or suchlike). Typically, it is non-functional, as in it is present only to space adjacent functional nucleic acid sequences from one another.

The term “pharmaceutically acceptable” as used herein is consistent with the art and means compatible with the other ingredients of the pharmaceutical composition and not deleterious to the recipient thereof.

By the terms “treat,” “treating” or “treatment of” (and grammatical variations thereof) it is meant that the severity of the subject's condition is reduced, at least partially improved or stabilized and/or that some alleviation, mitigation, decrease or stabilization in at least one clinical symptom is achieved and/or there is a delay in the progression of the disease or disorder.

The terms “prevent,” “preventing” and “prevention” (and grammatical variations thereof) refer to prevention and/or delay of the onset of a disease, disorder and/or a clinical symptom(s) in a subject and/or a reduction in the severity of the onset of the disease, disorder and/or clinical symptom(s) relative to what would occur in the absence of the methods of the invention. The prevention can be complete, e.g., the total absence of the disease, disorder and/or clinical symptom(s).

The prevention can also be partial, such that the occurrence of the disease, disorder and/or clinical symptom(s) in the subject and/or the severity of onset is substantially less than what would occur in the absence of the present invention.

A “treatment effective” amount as used herein is an amount that is sufficient to provide some improvement or benefit to the subject. Alternatively stated, a “treatment effective” amount is an amount that will provide some alleviation, mitigation, decrease or stabilization in at least one clinical symptom in the subject. Those skilled in the art will appreciate that the therapeutic effects need not be complete or curative, as long as some benefit is provided to the subject.

A “prevention effective” amount as used herein is an amount that is sufficient to prevent and/or delay the onset of a disease, disorder and/or clinical symptoms in a subject and/or to reduce and/or delay the severity of the onset of a disease, disorder and/or clinical symptoms in a subject relative to what would occur in the absence of the methods of the invention. Those skilled in the art will appreciate that the level of prevention need not be complete, as long as some preventative benefit is provided to the subject.

The phrase a “therapeutically effective amount” and like phrases mean a dose or plasma concentration in a subject that provides the desired specific pharmacological effect, e.g. to express a therapeutic gene in the liver, and secretion into the plasma. It is emphasized that a therapeutically effective amount may not always be effective in treating the conditions described herein, even though such dosage is deemed to be a therapeutically effective amount by those of skill in the art. The therapeutically effective amount may vary based on the route of administration and dosage form, the age and weight of the subject, and/or the disease or condition being treated.

The terms “individual,” “subject,” and “patient” are used interchangeably, and refer to any individual subject with a disease or condition in need of treatment. For the purposes of the present disclosure, the subject may be a primate, preferably a human, or another mammal, such as a dog, cat, horse, pig, goat, or bovine, and the like.

Additional patents incorporated for reference herein that are related to, disclose or describe an AAV or an aspect of an AAV, including the DNA vector that includes the gene of interest to be expressed are: U.S. Pat. Nos. 6,491,907; 7,229,823; 7,790,154; 7,201898; 7,071,172; 7,892,809; 7,867,484; 8,889,641; 9,169,494; 9,169,492; 9,441,206; 9,409,953; and, 9,447,433; 9,592,247; and, 9,737,618.

The invention provided herein can further be described in any of the following numbered paragraphs:
1. A recombinant adenovirus associated (rAAV) vector comprising in its genome:
a. 5′ and 3′ AAV inverted terminal repeats (ITR) sequences, and
b. located between the 5′ and 3′ ITRs, a heterologous nucleic acid sequence encoding all or a portion of an endogenous GAA signal peptide, a heterologous signal peptide and an alpha-glucosidase (GAA) polypeptide,
wherein the GAA polypeptide comprises amino acid residues 28-952 of SEQ ID NO: 1, 57-952 of SEQ ID NO:1, or comprises a N-terminal GAA polypeptide fragment comprising amino acids 28, 28-29, 28-30, 28-31, 28-32, or 28-33 of SEQ ID NO: 1 and a deletion of any number of amino acids from the next about 5 amino acids to about 40 amino acids after the N terminal GAA polypeptide fragment of SEQ ID NO: 1, and wherein the heterologous signal peptide can be inserted immediately after the N-terminal GAA polypeptide fragment and before the remaining amino acids of the GAA polypeptide, wherein the heterologous signal peptide is optionally fused at position 57 of the remaining amino acids of the GAA polypeptide, and where the GAA polypeptide can extend to amino acid 952 of SEQ ID NO: 1, or a functional fragment thereof, and
wherein the nucleic acid sequence encoding the GAA polypeptide can be codon optimized, and wherein the heterologous nucleic acid is operatively linked to a liver-specific promoter and a poly A sequence.
2. The recombinant AAV vector of paragraph 1, where the codon optimized nucleic acid sequence encoding the GAA polypeptide is selected from the group consisting of SEQ ID NO: 1-18, or functional fragment thereofs.
3. The recombinant AAV vector of any of the preceding paragraphs, which comprises the nucleic acid sequence of SEQ ID NO: 23, or a functional variant thereof.
4. The recombinant AAV vector of any of the preceding paragraphs, wherein the heterologous nucleic acid sequence encodes a GAA protein comprising a signal peptide fused to the GAA polypeptide, wherein the signal peptide is an endogenous GAA signal peptide, or a heterologous signal peptide, or a combination thereof.
5. The recombinant AAV vector of any of the preceding paragraphs, wherein the nucleic acid encoding SEQ ID NO: 3 is wildtype.
6. The recombinant AAV vector of any of the preceding paragraphs, further comprising at least one of a UTR, or a reverse RNA polII terminator sequence.
7. The recombinant AAV vector of any of the preceding paragraphs, wherein the AAV genome comprises, in the 5′ to 3′ direction:
a. a 5′ ITR,
b. a liver-specific promoter sequence,
c. an 5′ UTR sequence,
d. a nucleic acid encoding a portion or all of the endogenous GAA signal peptide,
e. a nucleic acid encoding a heterologous signal peptide or the N-terminal GAA polypeptide fragment,
f. a nucleic acid encoding an alpha-glucosidase (GAA) polypeptide, wherein the GAA polypeptide is functionally active,
g. a poly A sequence, and
h. a reverse RNA pol II terminator sequence.
8. The recombinant AAV vector of any of the preceding paragraphs, wherein the UTR is 5′ or 3′.
9. The recombinant AAV vector of any of the preceding paragraphs, wherein nucleic acid encoding the signal peptide encodes a signal sequence is selected from any of: an endogenous GAA signal peptide, a fibronectin signal peptide (FN1), a IL-2 wt signal peptide, modified IL-2 signal peptide, IL2(1-3) signal peptide, IgG signal peptide, a AAT signal peptide, a A2M signal peptide, or a PZP signal peptide, or an active fragment thereof having signal peptide activity.
10. The recombinant AAV vector of any of the preceding paragraphs, wherein the nucleic acid sequence encodes a GAA polypeptide having the amino acid sequence of SEQ ID NO: 1, or a polypeptide having at least 80% sequence identity to SEQ ID NO: 1 where amino acid residue 199 is a R (199R), amino acid residue 223 is a H (223H) and amino acid residue 780 is a I (780I).
11. The recombinant AAV vector of any of the preceding paragraphs, wherein the nucleic acid sequence encoding the GAA polypeptide is SEQ ID NO: 3, or a nucleic acid sequence having at least 80%, or at least 85%, or at least 90% sequence identity to SEQ ID NO: 3 that encodes a GAA polypeptide having at least 80% sequence identity to SEQ ID NO: 1 where amino acid residue 199 is a R (199R), amino acid residue 223 is a H (223H) and amino acid residue 780 is a I (780I).
12. The recombinant AAV vector of any of the preceding paragraphs, wherein the 5′ UTR sequence comprises SEQ ID NO: 41, or a nucleic acid having at least 80% sequence identity to SEQ ID NO: 41.
13. The recombinant AAV vector of any of the preceding paragraphs, wherein the 5′ UTR sequence comprises SEQ ID NO: 40, or a nucleic acid having at least 80% sequence identity to SEQ ID NO: 40.
14. The recombinant AAV vector of any of the preceding paragraphs, further comprising an intron sequence located 5′ of the nucleic acid sequence encoding the signal peptide, and 3′ of the promoter.
15. The recombinant AAV vector of any of the preceding paragraphs, wherein the intron sequence is selected from the group consisting of: MVM sequence, a HBB2 sequence, an CMVIE intron sequence, or a UBC intron sequence or a SV40 sequence.
16. The recombinant AAV vector of any of the preceding paragraphs, wherein the GAA polypeptide is a N-terminal truncated GAA polypeptide selected from any disclosed in Table 1.
17. The recombinant AAV vector of any of the preceding paragraphs, further comprising at least one polyA sequence located 3′ of the nucleic acid encoding the GAA gene and 5′ of the 3′ ITR sequence.
18. The recombinant AAV vector of any of the preceding paragraphs, wherein the heterologous nucleic acid sequence further comprises a 3′ UTR sequence, wherein the 3′ UTR sequence is located 3′ of the nucleic acid encoding the GAA polypeptide and 5′ of the 3′ ITR sequence, or is located between the nucleic acid encoding the GAA polypeptide and the poly A sequence, or RNA pol II terminator sequence.
19. The recombinant AAV vector of any of the preceding paragraphs, wherein the heterologous nucleic acid sequence further comprises a 3′ intron sequence, wherein the 3′ intron sequence is located 3′ of the nucleic acid encoding the GAA polypeptide and 5′ of the 3′ ITR sequence, or is located between the nucleic acid encoding the GAA polypeptide and the poly A sequence or RNA polII terminator sequence.
20. The recombinant AAV vector of any of the preceding paragraphs, wherein the ITR comprises an insertion, deletion or substitution.
21. The recombinant AAV vector of any of the preceding paragraphs, wherein one or more CpG islands in the ITR are removed.
22. The recombinant AAV vector of any of the preceding paragraphs, wherein
a. the nucleic acid encoding the signal peptide is selected from any of the group consisting of:
   AAT signal peptide (e.g., SEQ ID NO: 67), or an active fragment thereof having secretory signal activity, e.g., a nucleic acid encoding an amino acid sequence that has at least about 75%, or 80%, or 85%, or 90%, or 95%, or 98%, or 99% sequence identity to SEQ ID NO: 67;
   a fibronectin signal peptide (FN1) (e.g., SEQ ID NO: 73-75), or an active fragment thereof having secretory signal activity, e.g., a nucleic acid encoding an amino acid sequence that has at least about 75%, or 80%, or 85%, or 90%, or 95%, or 98%, or 99% sequence identity to SEQ ID NO: 73-75;
   an endogenous GAA signal peptide (SEQ ID NO: 51), or an active fragment thereof having secretory signal activity, e.g., a nucleic acid encoding an amino acid sequence that has at least about 75%, or 80%, or 85%, or 90%, or 95%, or 98%, or 99% sequence identity to SEQ ID NO: 51;
   an hIGF2 signal peptide (e.g., SEQ ID NO: 72), or an active fragment thereof having secretory signal activity, e.g., a nucleic acid encoding an amino acid sequence that has at least about 75%, or 80%, or 85%, or 90%, or 95%, or 98%, or 99% sequence identity to SEQ ID NO: 72;
   a IgG1 (201) signal peptide (SEQ ID NO: 54), or an active fragment thereof having secretory signal activity, e.g., a nucleic acid encoding an amino acid sequence that has at least about 75%, or 80%, or 85%, or 90%, or 95%, or 98%, or 99% sequence identity to SEQ ID NO: 54;
   wtIL2 leader peptide (SEQ ID NO: 55), or an active fragment thereof having secretory signal activity, e.g., a nucleic acid encoding an amino acid sequence that has at least about 75%, or 80%, or 85%, or 90%, or 95%, or 98%, or 99% sequence identity to SEQ ID NO: 55;
   mutant IL2 leader peptide (SEQ ID NO: 56) or an active fragment thereof having secretory signal activity, e.g., a nucleic acid encoding an amino acid sequence that has at least about 75%, or 80%, or 85%, or 90%, or 95%, or 98%, or 99% sequence identity to SEQ ID NO: 56; and
b. the nucleic acid encoding the GAA polypeptide is selected from any of the group consisting of: SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18 or a nucleic acid sequence having at least 60%, or 70%, or 80%, 85% or 90% or 95%, or 98%, or 99% sequence identity to SEQ ID NOS: 1-18.
23. The recombinant AAV vector of any of the preceding paragraphs, wherein the nucleic acid encoding the GAA polypeptide is selected from SEQ ID NO: 3, or a nucleic acid sequence having at least 60%, or 70%, or 80%, 85% or 90% or 95%, or 98%, or 99% sequence identity to SEQ ID NO: 3 which encodes a GAA polypeptide at least 85% sequence identity to SEQ ID NO: 1 where amino acid residue 199 is a R (199R), amino acid residue 223 is a H (223H) and amino acid residue 780 is a I (780I).
24. The recombinant AAV vector of any of the preceding paragraphs, wherein the nucleic acid encoding the GAA polypeptide encodes a GAA polypeptide beginning at any of amino acid residues 35, 40, 50, 57, 60, 68, 69, 70, 72, 74, 779, 790, 791, 792, 793, or 796 of SEQ ID NO: 1 or a sequence 80% identical to SEQ ID NO: 1 where amino acid residue 199 is a R (199R), amino acid residue 223 is a H (223H) and amino acid residue 780 is a I (780I).
25. The recombinant AAV vector of any of the preceding paragraphs, wherein the GAA polypeptide has an endogenous GAA signal peptide attached, or a heterologous signal peptide attached to the N-terminal of the GAA polypeptide, wherein the endogenous signal peptide has the amino acid sequence of SEQ ID NO: 59 or a sequence at least 80% sequence identity to SEQ ID NO: 59, and the heterologous signal peptide is selected from the group consisting of: SEQ ID NO: 60 (201 IgG signal peptide), or an IL2 wild type signal peptide (SEQ ID NO: 61), modified IL2 signal peptide (SEQ ID NO: 62), A2M signal peptide (SEQ ID NO: 63), or PZP signal peptide (SEQ ID NO: 64), or artificial signal peptide (SEQ ID NO: 65), or cathpetsin L signal peptide (SEQ ID NO: 66) or signal peptides at least 90% sequence identity to SEQ ID NOS: 60-66.
26. The recombinant AAV vector of any of the preceding paragraphs, wherein the liver specific promoter is selected from any of: SEQ ID NOS: 86, 88, 91-96, 146-150 or 439-441, or a liver specific promoter having at least 80% sequence identity to SEQ ID NOs: 86, 88, 91-96, 146-150 or 439-441.
27. The recombinant AAV vector of any of the preceding paragraphs, wherein the liver specific promoter is selected from any of: SEQ ID NOS: 98 or 99, or a liver specific promoter having at least 80% sequence identity to SEQ ID NOs: 98 or 99.
28. The recombinant AAV vector of any of the preceding paragraphs, wherein the liver specific promoter is SEQ ID NOS: 97, or a liver specific promoter having at least 80% sequence identity to SEQ ID NO: 97.
29. The recombinant AAV vector of any of the preceding paragraphs, wherein the recombinant vector is manufactured from the plasmid of SEQ ID NO: 27.
30. The recombinant AAV vector of any of the preceding paragraphs, wherein the nucleic acid comprises SEQ ID NO: 25, or a functional fragment thereof.
31. The recombinant AAV vector of any of the preceding paragraphs, wherein the recombinant AAV vector is a chimeric AAV vector, haploid AAV vector, a hybrid AAV vector or polyploid AAV vector.
32. The recombinant AAV vector of any of the preceding paragraphs, wherein the recombinant AAV vector is a rational haploid vector, a mosaic AAV vector, a chemically modified AAV vector, or a AAV vector from any AAV serotypes.
33. The recombinant AAV vector of any of the preceding paragraphs, wherein the recombinant AAV vector is selected from the group consisting of: a AAVXL32 vector, a AAVXL32.1 vector, a AAV8 vector, or a haploid AAV8 vector comprising at least one AAV8 capsid protein.
34. The recombinant AAV vector of any of the preceding paragraphs, wherein the serotype is AAV3b.
35. The recombinant AAV vector of any of the preceding paragraphs, wherein the AAV3b serotype comprises one or mutations in a capsid protein selected from any of: 265D, 549A, Q263Y.
36. The recombinant AAV vector of any of the preceding paragraphs, wherein the AAV3b serotype is selected from any of: AAV3b265D, AAV3b265D549A, AAV3b549A or AAV3bQ263Y, or AAV3bSASTG.
37. The recombinant AAV vector of any of the preceding paragraphs, wherein the poly A sequence is a full length HGF poly A sequence.
38. The recombinant AAV vector of any of the preceding paragraphs, wherein poly A sequence is selected from SEQ ID NO: 42, 43 or 44, or a nucleic acid sequence at least 80% sequence identity to SEQ ID NOS: 42-44.
39. The recombinant AAV vector of any of the preceding paragraphs, wherein the reverse RNA pol II terminator sequence is SEQ ID NO: 45, or a nucleic acid sequence at least 80% sequence identity to SEQ ID NOS: 45.
40. A pharmaceutical composition comprising the recombinant AAV vector of any one of the previous paragraphs in a pharmaceutically acceptable carrier.
41. A method to treat a subject with Pompe Disease, or a glycogen storage disease type II (GSD II, Acid Maltase Deficiency) or having a deficiency in alpha-glucosidase (GAA) polypeptide, comprising administering any of the recombinant AAV vector, or the rAAV genome or the nucleic acid sequence of any one of the previous paragraphs to the subject.
42. The method of paragraph 38, wherein the AAV vector manufactured from the plasmid of SEQ ID NO: 27.
43. The method of any of the preceding paragraphs, where the recombinant AAV vector comprises the nucleic acid sequence of SEQ ID NO: 3, or a functional fragment thereof.
44. The method of any of the preceding paragraphs, where the recombinant AAV vector comprises the nucleic acid sequence of SEQ ID NO: 23, or a functional variant thereof.
45. The method of any of the preceding paragraphs, wherein GAA polypeptide is secreted from the subject's liver and there is uptake of the secreted GAA by skeletal muscle tissue, cardiac muscle tissue, diaphragm muscle tissue or a combination thereof, wherein uptake of the secreted GAA results in a reduction in lysosomal glycogen stores in the tissue(s).
46. The method of any of the preceding paragraphs, wherein the administering to the subject is selected from any of: intramuscular, sub-cutaneous, intraspinal, intracisternal, intrathecal, intravenous administration.
47. The method of any of the preceding paragraphs, where the recombinant AAV vector is a chimeric AAV vector, haploid AAV vector, a hybrid AAV vector or polyploid AAV vector.
48. The method of any of the preceding paragraphs, where the recombinant AAV vector is a rational haploid vector, a mosaic AAV vector, a chemically modified AAV vector, or a AAV vector from any AAV serotypes.
49. The method of any of the preceding paragraphs, where the recombinant AAV vector is a AAVXL32 vector or a AAVXL32.1 vector or a AAV8 vector, or a haploid AAV8 vector comprising at least one AAV8 capsid protein.
50. The method of any of the preceding paragraphs, where the recombinant AAV vector is a AAV8 vector.
51. The method of any of the preceding paragraphs, where the recombinant AAV vector is administered at a dosage range of between 1.0E11 vg/kg and 5.0E13 vg/kg.
52. The method of any of the preceding paragraphs, further comprising receiving GAA protein enzyme replacement therapy, and withdrawing GAA protein enzyme replacement therapy (ERT) on the same day, a day after or, any time between day 1 and 26 weeks after administration of the recombinant AAV vector.
53. A nucleic acid construct comprising SEQ ID NO: 3, or a nucleic acid sequence at least 80% sequence identity to SEQ ID NOS: 3.
54. The nucleic acid of paragraph 50, wherein the expression of the nucleic acid sequence of SEQ ID NO: 3 or a nucleic acid having 80% sequence identity thereto encodes a GAA polypeptide having at least 80% sequence identity to SEQ ID NO: 1 and wherein there is R at position 199, a H at position 223 and I at position 780.
55. A nucleic acid construct comprising SEQ ID NO: 23, or a nucleic acid sequence at least 80% sequence identity to SEQ ID NO: 23.
56. The nucleic acid construct of paragraph 52, comprising SEQ ID NO: 3 or SEQ ID NO: 25, or a nucleic acid sequence at least 80% sequence identity to SEQ ID NOS: 3 or 25.
57. The nucleic acid of any of the preceding paragraphs, wherein the expression of the nucleic acid sequence of SEQ ID NO: 3 or a nucleic acid having 80% sequence identity thereto encodes a GAA polypeptide having at least 80% sequence identity to SEQ ID NO: 1 and wherein there is R at position 199, a H at position 223 and I at position 780.
58. A recombinant AAV comprising the nucleic acid construct of any of the preceding paragraphs.
59. The recombinant AAV vector of any of the preceding paragraphs, wherein the AAV lacks at least 1 amino acids of the GAA N terminus.
60. The recombinant AAV vector of any of the preceding paragraphs, wherein the AAV lacks at least 5, 10, 15, 20, 25, 30, 35, 40, 45, 50, 55 or more amino acids of the GAA N terminus.
61. The recombinant AAV vector of any of the preceding paragraphs, wherein the heterologous signal peptide is inserted immediately after the endogenous GAA signal peptide or a potion thereof.

### EXAMPLES

### Example 1: Generating rAAV Vectors

The rAAV genomes were packed into capsids to generate rAAV vectors using a rAAV producing cell line. Solely for proof of principal of rAAV vector construction, the capsids used were AAV3b capsids.

Making rAAV in the rAAV producing cell line: triple transfection technique was used to make rAAV in a suspension rAAV producer cell line, which can be scaled up for making clinical grade vector. Alternatively, different plasmids can be used, e.g., 1) pXX680-ad helper and 2) pXR3 the Rep and Cap 3) and the Transgene plasmid (ITR-transgene-ITR).

The rAAV genomes generated in Example 1 are used to generate rAVV vectors using a rAAV producing cell line, according to the methods as described in U.S. Pat. No. 9,441,206, which is incorporated herein in its entirety by reference. In particular, rAAV vectors or rAAV virions are produced using a method comprising: (a) providing a rAAV producing cell line an AAV expression system; (b) culturing the cells under conditions in which AAV particles are produced; and (c) optionally isolating the AAV particles. Ratios of triple transfection of the plasmid and transfection cocktail volumes can be optimized, with varying plasmid ratios of XX680, AAV rep/cap helper and TR plasmid to determine the optimal plasmid ratio for rAAV vector production.

In some instances, the cells are cultured in suspension under conditions in which AAV particles are produced. In another embodiment, the cells are cultured in animal component-free conditions. The animal component-free medium can be any animal component-free medium (e.g., serum-free medium) compatible with the rAAV producer cell line. Examples include, without limitation, SFM4Transfx-293 (Hyclone), Ex-Cell 293 (JRH Biosciences), LC-SFM (Invitrogen), and Pro293-S(Lonza). Conditions sufficient for the replication and packaging of the AAV particles can be, e.g., the presence of AAV sequences sufficient for replication of an rAAV genome described herein and encapsidation into AAV capsids (e.g., AAV rep sequences and AAV cap sequences) and helper sequences from adenovirus and/or herpesvirus.

Bacterial DNA sequences from the plasmid backbone can be packaged into AAV capsids during manufacturing of the recombinant AAV vectors leading to activations of the innate immune system through its interaction with TLR9 (Akira, 2006; Chadeuf, 2005; Wright, 2014). Various technologies can be used to eliminate plasmid backbone sequences in recombinant AAV preparations, for example minicircles which have limited scalability (Schnodt, 2016). Another method to avoid bacterial DNA sequence in the plasmid backbone is to use closed ended linear duplex DNA, which includes a range of DNA replication technology, including but not limited to doggy bone DNA (dbDNA™) for specifically manufacturing of recombinant AAV vectors. Using closed ended linear duplex DNA, such as dbDNA™ eliminates the bacterial backbone and has been used to produce vaccines and lentivirus (Walters et al, 2014; Scott et al, 2015; Karda et al, 2019) and was shown to be unable to trigger TLR9 responses by DNA vaccine developers.

Accordingly, in alternative embodiments, generation of rAAV vectors for use in the methods and compositions as disclosed herein can be performed using closed ended linear duplex DNA, including but not limited to Doggybone technology (dbDNA™), as disclosed in US Application 2018/0037943 and Karbowniczek et al., Bioinsights, 2017, which is incorporated herein in its entirety by reference. In brief, a plasmid for AAV production using a closed ended linear duplex DNA technology can comprise the ITRs, promoter and gene of interest, e.g., GAA as disclosed herein, is flanked by a 56 bp palindromic protelomerase recognition sequence. The plasmid is denatured, and in the presence of a Phi29 DNA polymerase, and appropriate primers, Phi29 initiates rolling circle amplification (RCA), creating a double stranded cancatameric repeats of the original construct. When protelomerase is added, binding of the palindromic protelomerase recognition sequences occurs and cleavage-joining reaction occurs to result in a monomeric double stranded (ds) linear covalently closed DNA construct. Addition of common restriction enzymes remove the undesired DNA plasmid backbone sequence and digestion with exonuclease activity, resulting in dbDNA which can be size fractionated to isolate the dbDNA sequence encoding the ITRs, promoter and gene of interest. An exemplary plasmid for generation of rAAV vectors using closed ended linear duplex DNA such as dbDNA™ technology, comprises in the following 5′ to 3′ direction: 5′-protelomerase RS, 5′ITR, LSP promoter, GAA (e.g., wild-type or codon optimized), 3′UTR, hGH poly(A), 3′ ITR, 3′-protelomerase RS (sense strand), where the sense strand is linked to the complementary antisense strand for a stranded (ds) linear covalently closed DNA construct. The use of closed ended linear duplex DNA, e.g., doggy bone DNA (dbDNA™) as a starting material for the manufacturing of an AAV vector for use in the methods and composition as disclosed herein eliminates the bacterial backbone used to propagate the plasmid containing AAV vector with an inability for the product to trigger Toll-like receptor 9 (TLR9) responses. An exemplary dbDNA plasmid for use in the manufacturing of a rAAV vector for use in the methods and compositions as disclosed herein is SEQ ID NO: 37. In some embodiments, the 4780 bp fragment of 347-5126 bp of SEQ ID NO: 37 (i.e., the ITR-to-ITR sequence of SEQ ID NO: 37) can be replaced by any ITR-to-ITR sequence as disclosed herein, e.g., a ITR-to-ITR sequence selected from any of: SEQ ID NO: 23, SEQ ID NO: 24, SEQ ID NO: 31-35 as disclosed herein. Use of closed ended linear duplex DNA technology for manufacturing further reduce the risk for liver enzyme elevations observed at a dose of 1.6E13 vg/kg in patients with Pompe disease.

### Example 2: Comparison of the M3 Construct and Actus 101

Study Objective: The primary objective of this study presented herein in this example is to evaluate a series of gene therapy vector variants for tissue biodistribution and expression of human acid glucosidase alpha (GAA) in a mouse model of Pompe Disease. The following vectors ACTUS 101 (lot AB20200915), M3 dbp (lot AB20200914) and M3 db (lot AB20201117) were included in this study.

Background: ACTUS-101 (AAV2/8-LSPhGAA) (SEQ ID NO:451) is an infectious non-replicating recombinant adeno-associated viral vector (AAV) serotype 8, pseudotyped with AAV2 inverted terminal repeats (ITR), expressing human GAA under the control of a liver specific promoter (LSP). The GAA has an amino acid composition that is the same as in the FDA approved Enzyme Replacement Therapy Myozyme/Lumizyme for the treatment of Pompe Disease.

The Original ACTUS-101 transgene cassette was designed without codon optimization. Several design iterations have been made since the original design and have characterized these in a series of in vivo studies in wild type mice and in GAA knockout (KO) mice.

An additional change includes the use of synthetic doggy bone DNA (dbDNA™) as a starting material for the manufacturing of the gene therapy vector, eliminating the bacterial backbone and thus minimizing the ability of the product to trigger Toll-like receptor 9 (TLR9) responses.

The M3 vector (SEQ ID NO: 37) in this study uses the same AAV8 capsid as in ACTUS-101 and is made using the doggy bone precursor plasmid (dbp) for lot AB20200914 and doggy bone DNA (db) for lot AB20201117.

TABLE 5
Study design:

| Mouse strain | GAA −/− mice, JAX strain 004154, B6; 129-Gaa<tm1Rabn>/J |
|---|---|
| Sex | Male |
| Age | 5-7 weeks |
| Mice per vector | N = 5 |
| Doses per vector | 3e10 vg/mouse (LOW); 3e11 vg/mouse dose 2 (HIGH) |
| Injection Volume | 150 ul |
| Route of Administration | tail vein injection |
| Necropsy | 28 days post-injection |
| Number of vectors | 3 |
| Post-harvest analysis | GAA expression in sera, GAA activity in sera, GAA uptake by |
| | target tissues, GAA activity in target tissues, glycogen reduction |
| | in target tissues |

TABLE 6
Treatment groups:

Number of mice

| Group | Vector | 3e10* | 3e11* |
|---|---|---|---|
| 1 | SALINE | 5 | N/A |
| 2 | ACTUS 101 | 5 | 5 |
| 3 | M3 dbp | 5 | 5 |
| 4 | M3 db | 5 | 5 |

*Vector doses in vector genomes/mouse

Postharvest analysis: GAA expression in sera over time by western blotting and semi-quantitative densitometry; GAA expression in liver by western blotting and semi-quantitative densitometry GAA uptake by select tissues (heart and diaphragm) by western blotting and semi-quantitative densitometry, GAA enzymatic activity in sera over time by 4MU assay; GAA enzymatic activity in liver by 4MU assay; GAA uptake by select tissues (heart, diaphragm and quad) by 4MU assays; glycogen content in select tissues (heart, diaphragm and quad).

### Materials and Methods

Cloning and vector quantification: All vectors were made using methods as described in Example 1. The constructs were packaged in the AAV8 viral capsid and titered by digital droplet PCR (ddPCR) method using primers directed at vector ITRs.

Tissue preservation: Fresh tissue and sera specimens were immediately frozen and stored at −80° C. until use for molecular biology analyses.

Analysis of GAA protein levels in tissues by Western Blot and Densitometry: Tissues were homogenized in T-PER buffer (ThermoFisher 78510) with Halt Protease Inhibitor Cocktail (Thermo 78430) in TissueLyser and protein was quantified by BCA assay. Samples were heated at 95 degrees C. for 5 minutes and 50 μg protein was loaded onto Novex Wedge Well 4-12% Tris/Glycine gel (Invitrogen LC2675), run at 225V for 40 minutes using Tris Glycine Running buffer (ThermoFisher) and transferred onto iBlot2 NC Mini Stacks (Thermo IB23002) using the PO program (20 V 1 min, 23V 4 min, 25V 2 min). Membranes were washed in PBST and stained with Ponceau S stain (Sigma P7170) for 5 minutes followed by washing 3× with distilled water.

Membranes were imaged on iBright imager (FL15000) using the Ponceau S setting and destained using 0.1M NaOH for 30 sec followed by rinsing the membrane with water for 2-3 minutes. Blocking was done in Superblock (TBS) blocking solution (ThermoFisher 37536) for 1 hour at room temperature. Acid-α-glucosidase (GAA) protein detection was obtained after incubation overnight at 4° C. with a rabbit anti-GAA antibody (AbCam 137068) diluted 1:8000 in PBS 0.05% tween 20, followed by a goat anti-rabbit HRP conjugated antibody (Abcam ab205718) diluted 1:10000. The HRP enzyme activity was detected by Clarity Enhanced Chemiluminescence (ECL) Western Blotting Substrate (BioRad 1705061). The images were acquired by iBright imaging system, densitometry was performed on iBright software v. 4.0.1 and results were expressed as relative calculation (ratio) of the intensity of GAA antibody detected band per total protein by Ponceau S staining. For liver samples, rhGAA was run as a standard curve for absolute quantification.

Measurement of GAA secretable protein levels in blood by Western Blot (WB) and Densitometry: Fresh blood samples obtained from submandibular bleed were centrifuged and serum collected. 1.5 ul serum was diluted in 18.5 ul T-PER buffer (ThermoFisher 78510) and 20 ul 2× sample buffer (Sigma S3401) and heated at 95 degrees C. for 5 minutes. Samples (40 μl) were run onto Novex Wedge Well 4-12% Tris/Glycine gel (Invitrogen LC2675) at 225V for 40 minutes using Tris Glycine Running buffer (ThermoFisher) and transferred onto iBlot2 NC Mini Stacks (Thermo IB23002) using the PO program (20 V 1 min, 23V 4 min, 25V 2 min). Membranes were washed in PBST and stained with Ponceau S stain (Sigma P7170) for 5 minutes followed by washing 3× with distilled water. Membranes were imaged on iBright imager (FL15000) using the Ponceau S setting and destained using 0.1M NaOH for 30 sec followed by rinsing the membrane with water for 2-3 minutes. Blocking was done in Superblock (TBS) blocking solution (ThermoFisher 37536) for 1 hour at room temperature. Acid-α-glucosidase (GAA) protein detection was obtained after incubation overnight at 4° C. with a rabbit anti-GAA antibody (AbCam 137068) diluted 1:8000 in PBS 0.05% tween 20, followed by a goat anti-rabbit HRP conjugated antibody (Abcam ab205718) diluted 1:10000. The HRP enzyme activity was detected by Clarity Enhanced Chemiluminescence (ECL) Western Blotting Substrate (BioRad 1705061). The images were acquired by the image analyzer iBright imaging system, densitometry was performed on iBright software v. 4.0.1 and results were given by relative calculation (ratio) of the intensity of GAA antibody detected band per total protein by Ponceau S staining.

GAA activity measurement in tissues: GAA activity was measured on frozen tissues following homogenization and sonication of tissue samples in distilled water. Depending upon the tissue size, 10-50 mg tissue was weighed and homogenized, the homogenates were sonicated at 4 degree c. 3 times for 15 seconds, then centrifuged for 3 min at 15000 RPM. For serum GAA, 10 ul was analyzed, with or without 80 μM acarbose. The reaction was set up with 10 ul of supernatant and 20 ul of substrate-4MUα-D-glucoside, in a 96 wells plate (VWR62402-970). The reaction mixture was incubated at 37 degrees C. for one hour and was stopped by adding 130 ul of Sodium Carbonate buffer pH10.5. A standard curve (0-1000 pmol/ul of 4MU) was used to measure released fluorescent 4MU from individual reaction mixture, using TECAN GENios microplate reader at 465 nm (Emission) and 360 nm (excitation). The protein concentrations of the clarified supernatants were quantified via the Bradford assay (Bio-Rad Laboratories, Cat No. 500-0006). GAA activity was measured in the tissue homogenates by conversion of the artificial substrate 4-methylumbelliferyl (4-MU) α-D-glucoside to the fluorescent product umbelliferone at acidic pH 4.3 as described [1]. To calculate the GAA activity, released 4MU concentration was divided by the sample protein concentration and activity was reported as nmol/hour/mg protein. QA and QC samples were run on the same plate for experimental assay controls.

GAA activity measurement in serum: Fresh blood samples obtained from submandibular bleed were centrifuged and serum collected. 10 ul of serum was treated 2 ul of 800 μM acarbose. The reaction was set up with 10 ul of supernatant and 20 ul of substrate-4MUα-D-glucoside, in a 96 wells plate (VWR62402-970). The reaction mixture was incubated at 37 degrees C. for one hour and was stopped by adding 130 ul of Sodium Carbonate buffer pH10.5. A standard curve of rhGAA (R&D Systems, Cat. No 8329-GH) Standards, 2,000 ng/mL to 25 ng/mL was used. Released fluorescent 4MU from individual reaction mixture was measured using BioTek FLx800 Reader, Excitation filter=360/40 and Emission filter=460/40. GAA activity was determined by interpolation to the standard curve with a 4-parameter curve fit model and activity was reported as ng/ml.

Glycogen content: Glycogen content of tissues was measured indirectly as the glucose released after total digestion by amyloglucosidase of the tissue homogenates using the Aspergillus niger assay system and the glucose reagent (Infinity Glucose; TR15421, Thermo Scientific, VA, USA) in a standardized reaction using the Aspergillus niger assay system. The same tissue homogenates used above were used to measure total glycogen content in each tissue. The reaction was set up with 20 ul of supernatant and 55 ul distilled water. Samples were boiled for 3 min and immediately cooled on ice for 10 min. 25 ul of amyloglucosidase (1:50 in 0.1M potassium acetate pH5.5) was added to each reaction tube. A reaction control tube without any amyloglucosidase (homogenate+water) was also set up for each reaction tube. Both sample tube and control sample reaction tubes were incubated at 37 degrees C. for 90 minutes and reaction, was stopped by boiling the tubes for 3 min followed by centrifugation for 3 min at 15000 RPM. The glucose released from each reaction tube was determined by adding 30 ul of supernatant into 1 ml of Infinity glucose reagent (TR15421) and measuring on spectrophotometer at 340 nm (Shimadzu UV-1700).

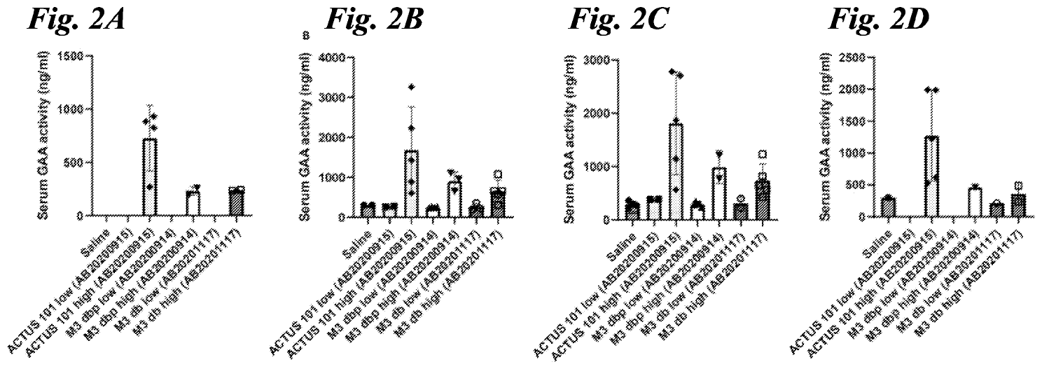

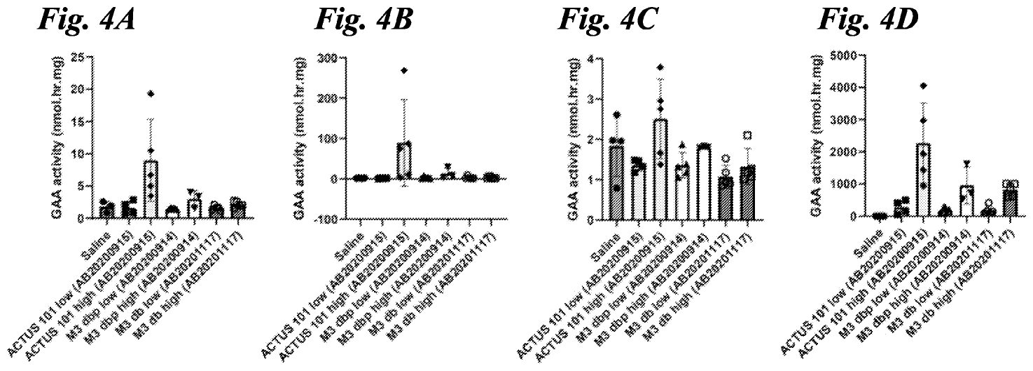

Observations: As observed in FIGS. 1A-1D, expression of GAA in cardiac serum was not significantly higher when driven from the M3 construct as compared to Actus 101 over a 4 week period of time. In fact, GAA expression from the M3 construct was reduced at week 4 (FIG. 1D) as compared to week 3 (FIG. 1C), whereas GAA expression from Actus 101 was sustained for at least 4 weeks. Similarly, as observed in FIGS. 2A2-D, the activity of GAA in cardiac serum was not significantly higher from the M3 construct (high or low dose) as compared to Actus 101 over a 4 week period of time. Further, data presented herein showed that the M3 construct was similarly unable to increase GAA levels and activity in target tissues, such as the heart, diaphragm, quadriceps muscle and liver, following administration as compared to Actus 101 (see, e.g., FIGS. 3A-4D).

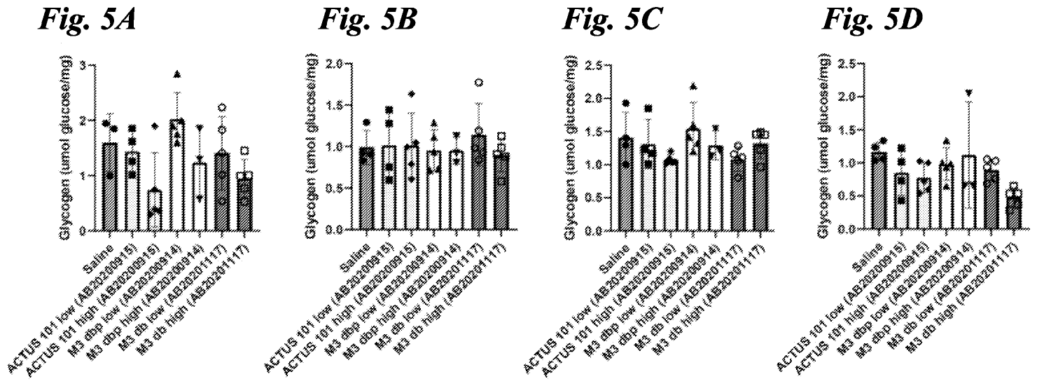

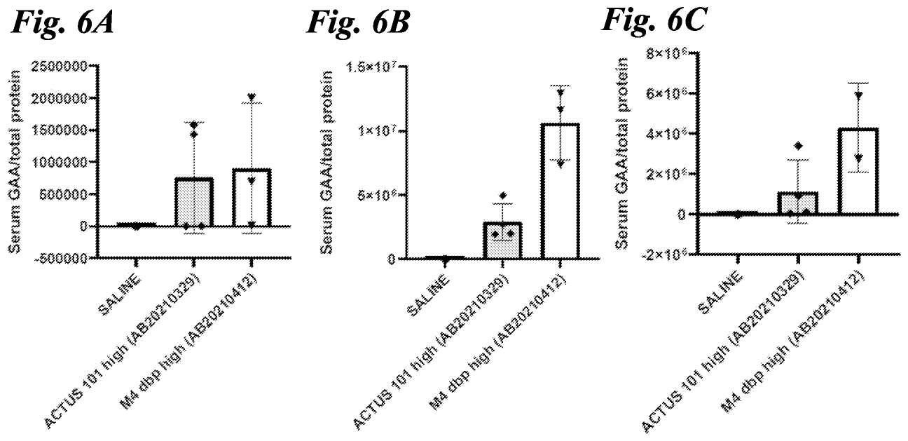

Additionally, as shown in FIGS. 5A-5D, a reduction in glycogen did not track in a dose dependent manner in mice following administration of the M3 construct. A significant dose dependent reduction of glycogen reduction was observed in the heart, diaphragm, quadriceps muscle and liver in mice administration Actus 101, however, this trend was not observed following administration of the M3 construct.

Accordingly, the data presented herein in this Example indicates that the M3 construct fails to perform better than Actus 101 with regards to increasing GAA protein levels and activity that can be sustained over a long period of time (e.g., more than 4 weeks) and that was capable of inducing glycogen clearance in the cell.

Further, additional variants based on the Actus 101 construct were developed and tested for their ability to promote glycogen clearance in the cell. Such constructs include pP110 and pP113. As shown in FIG. 41, pP110 exhibited a superior ability to promote glycogen clearance from the heart as compared to Actus 101 and pP113—a marked reduction of glyocen levels are observed in the heart following administration of pP110. A greater reduction of glycogen was observed when the pP110 was administered at a dose of 3E11 as compared to an administration of 3E10, confirming that this effect is dosage dependent.

### Example 3: Comparison of the M4 Construct and Actus 101

Study Objective: The primary objective of this study presented herein in this example is to evaluate a series of gene therapy vector variants for tissue biodistribution and expression of human acid glucosidase alpha (GAA) in a mouse model of Pompe Disease. The following vectors ACTUS 101 (lot AB20210329) and M4 dbp (lot AB20210412) were included in this study.

Background: ACTUS-101 (AAV2/8-LSPhGAA) is an infectious non-replicating recombinant adeno-associated viral vector (AAV) serotype 8, pseudotyped with AAV2 inverted terminal repeats (ITR), expressing human GAA under the control of a liver specific promoter (LSP). The GAA has an amino acid composition that is the same as in the FDA approved Enzyme Replacement Therapy Myozyme/Lumizyme for the treatment of Pompe Disease

This study compares Modification 4 (M4) vector to ACTUS-101. The specific elements that were removed include all Cytosine-phosphate-Guanine (CpG) dinucleotides found within the protein coding sequence, and a remnant fragment of the AAV P5 promoter located upstream of the LSP promoter. M4 also includes an RNA polymerase II termination sequence between the poly(A) signal and the 3′ITR to prevent the potential formation of the double stranded RNA.

An additional change includes the use of synthetic doggy bone DNA (dbDNA™) as a starting material for the manufacturing of the gene therapy vector, eliminating the bacterial backbone and thus minimizing the ability of the product to trigger Toll-like receptor 9 (TLR9) responses.

The M4 vector in this study uses the same AAV8 capsid as in ACTUS-101 and is made using the doggy bone precursor plasmid (dbp).

TABLE 7
Study design:

| Mouse strain | GAA −/− mice, JAX strain 004154, B6; 129-Gaa<tm1Rabn>/J |
|---|---|
| Sex | Male |
| Age | 7-12 weeks |
| Mice per vector | N = 4-8 |
| Doses per vector | 3e10 vg/mouse Low 3e11 vg/mouse High |
| Injection Volume | 150 ul |
| Route of Administration | tail vein injection |
| Necropsy | 21 days post-injection (3-week sacrifice) and 56 days post- |
| | injection (8-week sacrifice) |
| Number of vectors | 2 |
| Post-harvest analysis | GAA expression in sera, GAA activity in sera, GAA uptake by |
| | target tissues, GAA activity in target tissues, glycogen reduction |
| | in target tissues |

TABLE 8
Treatment groups:

| | | Number of mice |
|---|---|---|
| Group | Vector | 3e11* |
| 1 | SALINE | 4 |
| 2 | ACTUS 101 | 4 |
| 3 | M4 dbp | 4 |
| 4 | M4 dbp | 4 |

*Vector doses in vector genomes/mouse

Postharvest analysis: GAA expression in sera over time by western blotting and semi-quantitative densitometry; GAA uptake by target organs (heart, diaphragm) by western blotting and semi-quantitative densitometry, GAA enzymatic activity in sera over time by 4MU assay; GAA enzymatic activity in liver by 4MU assay; GAA uptake by select tissues (heart, diaphragm) by 4MU assays; glycogen content in select tissues (heart, diaphragm).

### Materials and Methods

Cloning and vector quantification: All vectors were made using methods as described in Example 1. The constructs were packaged in the AAV8 viral capsid and titered by digital droplet PCR (ddPCR) method using primers directed at vector ITRs.

Tissue preservation: Fresh tissue and sera specimens were immediately frozen and stored at −80° C. until use for molecular biology analyses.

Analysis of GAA protein levels in tissues by Western Blot and Densitometry: Tissues were homogenized in T-PER buffer (ThermoFisher 78510) with Halt Protease Inhibitor Cocktail (Thermo 78430) in TissueLyser and protein was quantified by BCA assay. Samples were diluted in T-PER buffer (ThermoFisher 78510) and 2× sample buffer (Sigma S3401), heated at 95 degrees C. for 5 minutes and 50 μg protein was loaded onto Novex Wedge Well 4-12% Tris/Glycine gel (Invitrogen LC2675), run at 225V for 40 minutes using Tris Glycine Running buffer (ThermoFisher) and transferred onto iBlot2 NC Mini Stacks (Thermo IB23002) using the PO program (20 V 1 min, 23V 4 min, 25V 2 min). Membranes were washed in PBST and stained with Ponceau S stain (Sigma P7170) for 5 minutes followed by washing 3× with distilled water. Membranes were imaged on iBright imager (FL15000) using the Ponceau S setting and destained using 0.1M NaOH for 30 sec followed by rinsing the membrane with water for 2-3 minutes. Blocking was done in Superblock (TBS) blocking solution (ThermoFisher 37536) for 1 hour at room temperature. Acid-α-glucosidase (GAA) protein detection was obtained after incubation overnight at 4° C. with a rabbit anti-GAA antibody (AbCam 137068) diluted 1:8000 in PBS 0.05% tween 20, followed by a goat anti-rabbit HRP conjugated antibody (Abcam ab205718) diluted 1:10000. The HRP enzyme activity was detected by Clarity Enhanced Chemiluminescence (ECL) Western Blotting Substrate (BioRad 1705061). The images were acquired by iBright imaging system, densitometry was performed on iBright software v. 4.0.1 and results were expressed as relative calculation (ratio) of the intensity of GAA antibody detected band per total protein by Ponceau S staining. For liver samples, rhGAA was run as a standard curve for absolute quantification.

Measurement of GAA secretable protein levels in blood by Western Blot (WB) and Densitometry: Fresh blood samples obtained from submandibular bleed were centrifuged and serum collected. 1.5 ul serum was diluted in 18.5 ul T-PER buffer (ThermoFisher 78510) and 20 ul 2× sample buffer (Sigma S3401) and heated at 95 degrees C. for 5 minutes. Samples (40 μl) were run onto Novex Wedge Well 4-12% Tris/Glycine gel (Invitrogen LC2675) at 225V for 40 minutes using Tris Glycine Running buffer (ThermoFisher) and transferred onto iBlot2 NC Mini Stacks (Thermo IB23002) using the PO program (20 V 1 min, 23V 4 min, 25V 2 min). Membranes were washed in PBST and stained with Ponceau S stain (Sigma P7170) for 5 minutes followed by washing 3× with distilled water. Membranes were imaged on iBright imager (FL15000) using the Ponceau S setting and destained using 0.1M NaOH for 30 sec followed by rinsing the membrane with water for 2-3 minutes. Blocking was done in Superblock (TBS) blocking solution (ThermoFisher 37536) for 1 hour at room temperature. Acid-α-glucosidase (GAA) protein detection was obtained after incubation overnight at 4° C. with a rabbit anti-GAA antibody (AbCam 137068) diluted 1:8000 in PBS 0.05% tween 20, followed by a goat anti-rabbit HRP conjugated antibody (Abcam ab205718) diluted 1:10000. The HRP enzyme activity was detected by Clarity Enhanced Chemiluminescence (ECL) Western Blotting Substrate (BioRad 1705061). The images were acquired by the image analyzer iBright imaging system, densitometry was performed on iBright software v. 4.0.1 and results were given by relative calculation (ratio) of the intensity of GAA antibody detected band per total protein by Ponceau S staining.

GAA activity measurement in tissues: GAA activity was measured on frozen tissues following homogenization and sonication of tissue samples in distilled water. Depending upon the tissue size, 10-50 mg tissue was weighed and homogenized, the homogenates were sonicated at 4 degree c. 3 times for 15 seconds, then centrifuged for 3 min at 15000 RPM. For serum GAA, 10 ul was analyzed, with or without 80 μM acarbose. The reaction was set up with 10 ul of supernatant and 20 ul of substrate-4MUα-D-glucoside, in a 96 wells plate (VWR62402-970). The reaction mixture was incubated at 37 degrees C. for one hour and was stopped by adding 130 ul of Sodium Carbonate buffer pH10.5. A standard curve (0-1000 pmol/ul of 4MU) was used to measure released fluorescent 4MU from individual reaction mixture, using TECAN GENios microplate reader at 465 nm (Emission) and 360 nm (excitation). The protein concentrations of the clarified supernatants were quantified via the Bradford assay (Bio-Rad Laboratories, Cat No. 500-0006). GAA activity was measured in the tissue homogenates by conversion of the artificial substrate 4-methylumbelliferyl (4-MU) α-D-glucoside to the fluorescent product umbelliferone at acidic pH 4.3 as described [1]. To calculate the GAA activity, released 4MU concentration was divided by the sample protein concentration and activity was reported as nmol/hour/mg protein. QA and QC samples were run on the same plate for experimental assay controls.

GAA activity measurement in serum: Fresh blood samples obtained from submandibular bleed were centrifuged and serum collected. 10 ul of serum was treated 2 ul of 800 μM acarbose. The reaction was set up with 10 ul of supernatant and 20 ul of substrate-4MUα-D-glucoside, in a 96 wells plate (VWR62402-970). The reaction mixture was incubated at 37 degrees C. for one hour and was stopped by adding 130 ul of Sodium Carbonate buffer pH10.5. A standard curve of rhGAA (R&D Systems, Cat. No 8329-GH) Standards, 2,000 ng/mL to 25 ng/mL was used. Released fluorescent 4MU from individual reaction mixture was measured using BioTek FLx800 Reader, Excitation filter=360/40 and Emission filter=460/40. GAA activity was determined by interpolation to the standard curve with a 4-parameter curve fit model and activity was reported as ng/ml.

GAA activity measurement in serum-alternative method: GAA, 10 ul was analyzed, with or without 80 μM acarbose. The reaction was set up with 10 ul of supernatant and 20 ul of substrate-4MUα-D-glucoside, in a 96 wells plate (VWR62402-970). The reaction mixture was incubated at 37 degrees C. for one hour and was stopped by adding 130 ul of Sodium Carbonate buffer pH10.5. A standard curve (0-1000 pmol/ul of 4MU) was used to measure released fluorescent 4MU from individual reaction mixture, using TECAN GENios microplate reader at 465 nm (Emission) and 360 nm (excitation). The protein concentrations of the clarified supernatants were quantified via the Bradford assay (Bio-Rad Laboratories, Cat No. 500-0006). GAA activity was measured in the tissue homogenates by conversion of the artificial substrate 4-methylumbelliferyl (4-MU) α-D-glucoside to the fluorescent product umbelliferone at acidic pH 4.3 as described [1]. To calculate the GAA activity, released 4MU concentration was divided by the sample protein concentration and activity was reported as nmol/hour/mg protein. A QA and QC samples were run on the same plate for experimental assay controls.

Glycogen content: Glycogen content of tissues was measured indirectly as the glucose released after total digestion by amyloglucosidase of the tissue homogenates using the Aspergillus niger assay system and the glucose reagent (Infinity Glucose; TR15421, Thermo Scientific, VA, USA) in a standardized reaction using the Aspergillus niger assay system. The same tissue homogenates used above were used to measure total glycogen content in each tissue. The reaction was set up with 20 ul of supernatant and 55 ul distilled water. Samples were boiled for 3 min and immediately cooled on ice for 10 min. 25 ul of amyloglucosidase (1:50 in 0.1M potassium acetate pH5.5) was added to each reaction tube. A reaction control tube without any amyloglucosidase (homogenate+water) was also set up for each reaction tube. Both sample tube and control sample reaction tubes were incubated at 37 degrees C. for 90 minutes and reaction, was stopped by boiling the tubes for 3 min followed by centrifugation for 3 min at 15000 RPM. The glucose released from each reaction tube was determined by adding 30 ul of supernatant into 1 ml of Infinity glucose reagent (TR15421) and measuring on spectrophotometer at 340 nm (Shimadzu UV-1700).

### Observations:

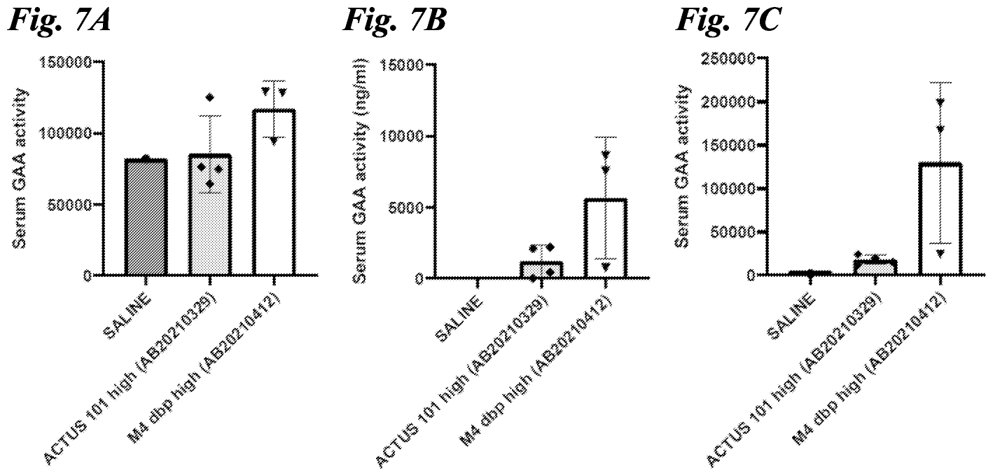

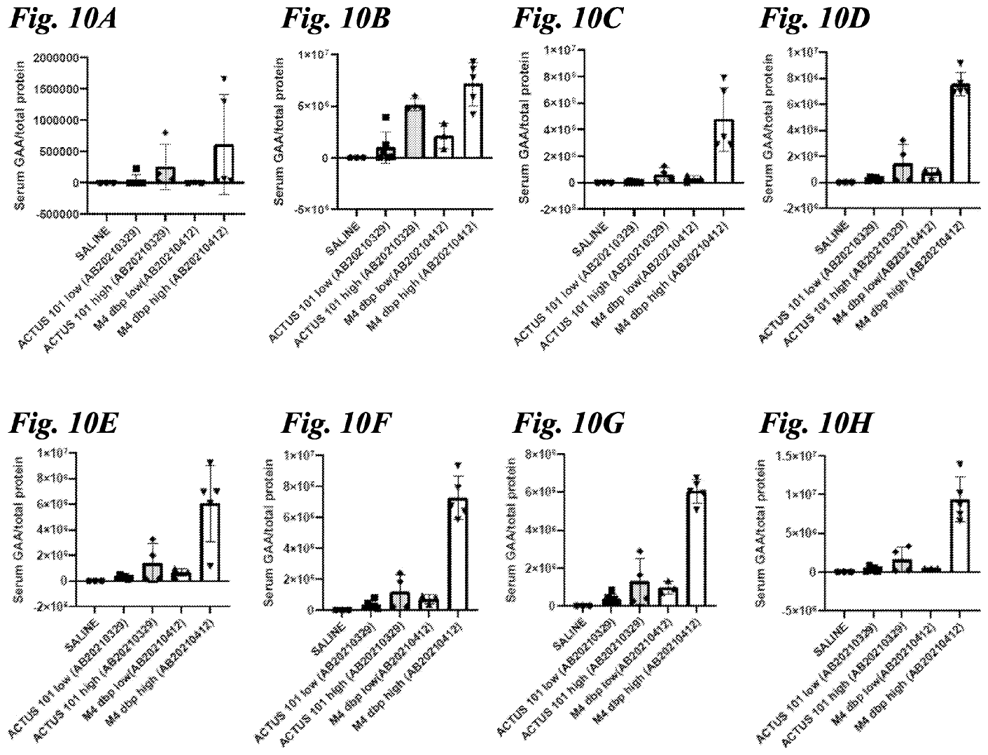

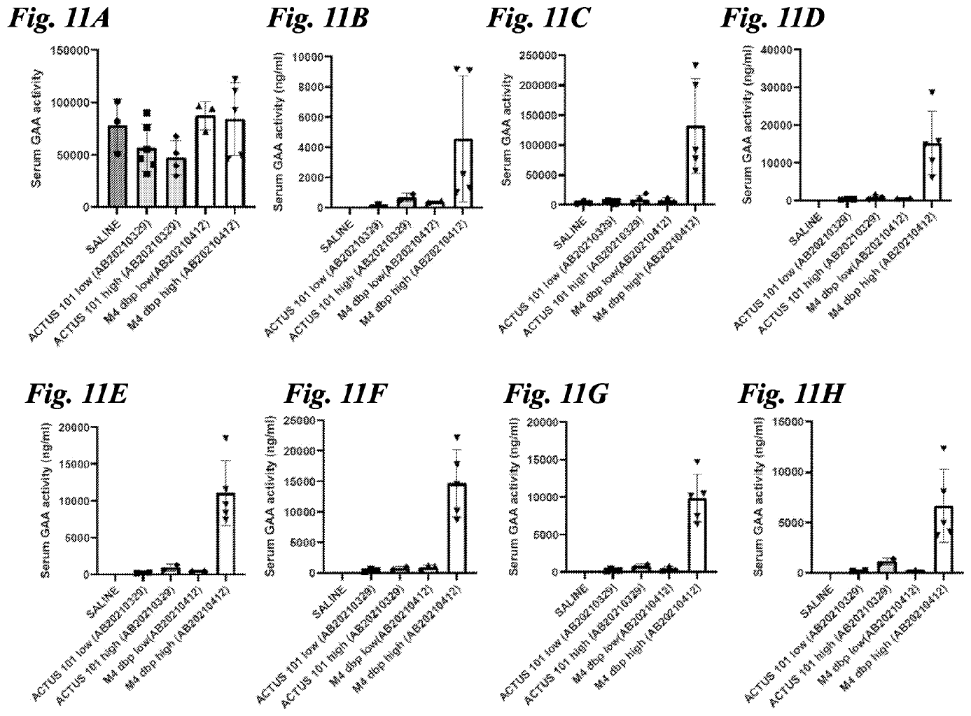

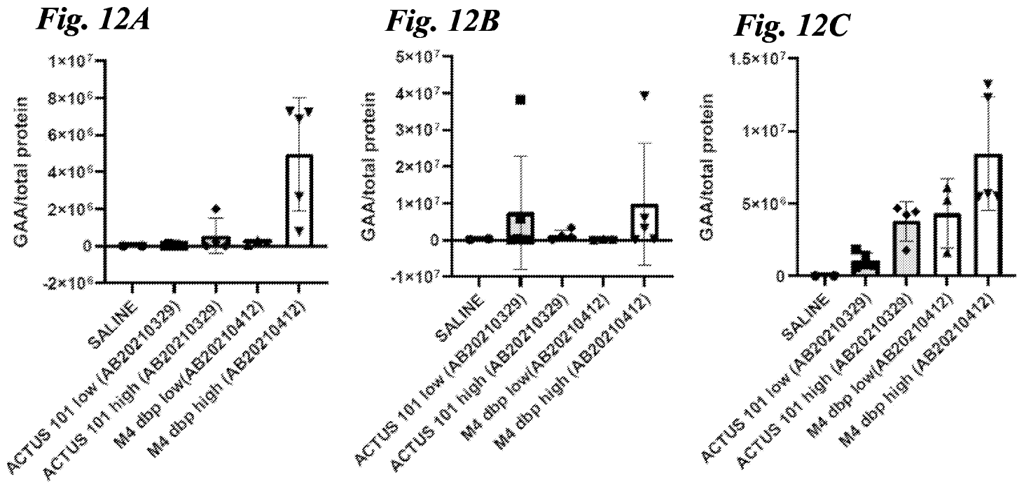

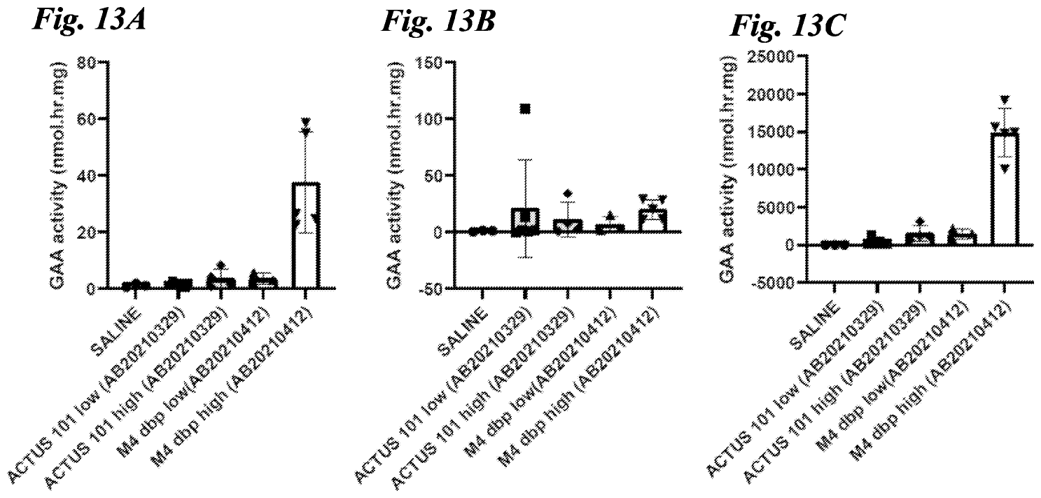

Expression of GAA protein levels in serum was markedly higher in mice that were administered the M4 construct as compared to those administered Actus 101. This was observed at 3 weeks and 8 weeks post administration (FIGS. 6A-6C and 10A-10H, respectively). This increased level of GAA protein following administration of M4 translated to a marked increase in GAA activity present in the serum in mice; increased GAA activity was observed at 3 weeks and 8 weeks post administration (FIGS. 7A-7C and 11A-11H, respectively). Similar results were additionally observed in numerous target tissues. Increased GAA protein levels and GAA activity were observed in the heart, diaphragm and liver at 3 weeks post administration and up to 8 weeks post administration (see FIGS. 8A-8C, 12A-12C and 13A-13C, respectively).

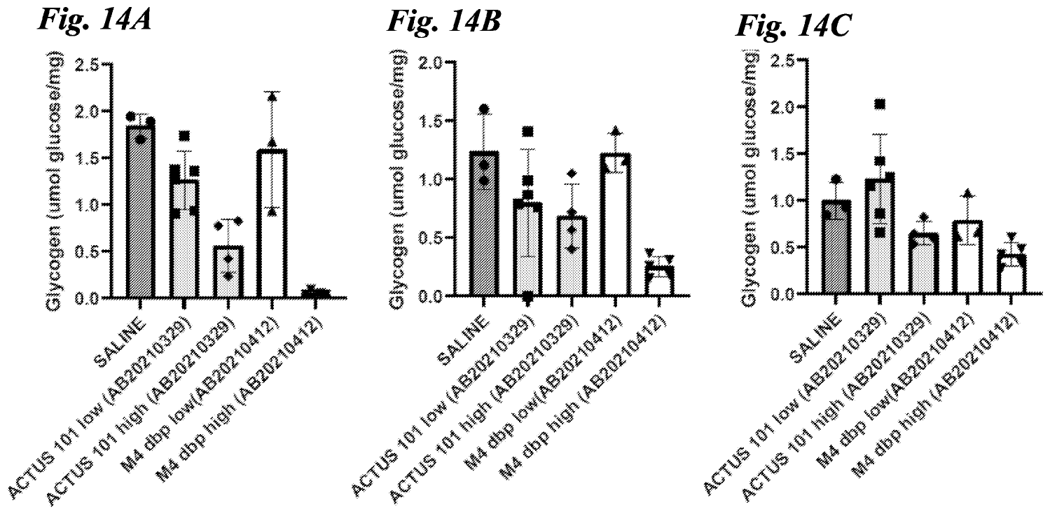

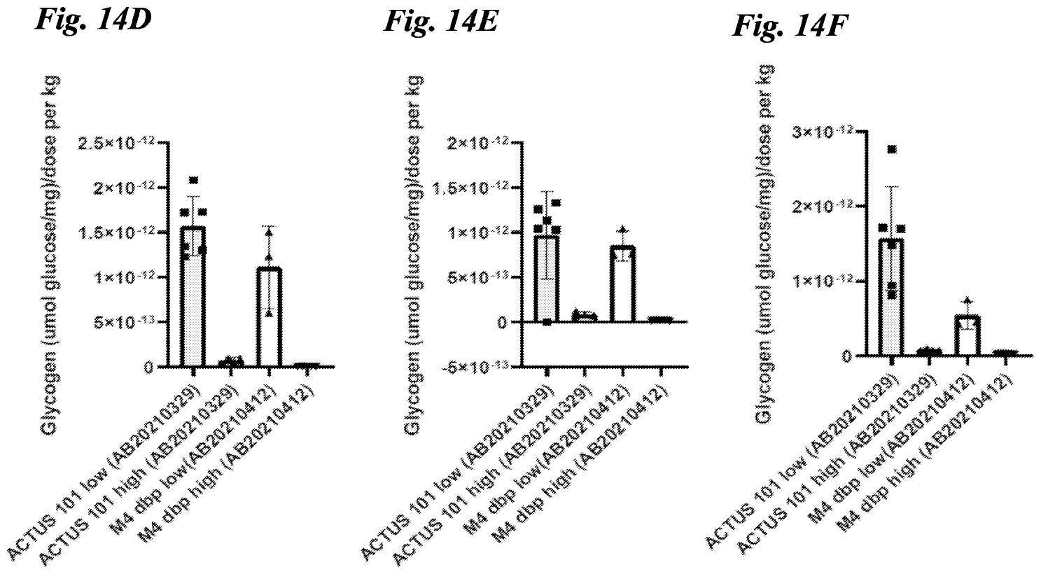

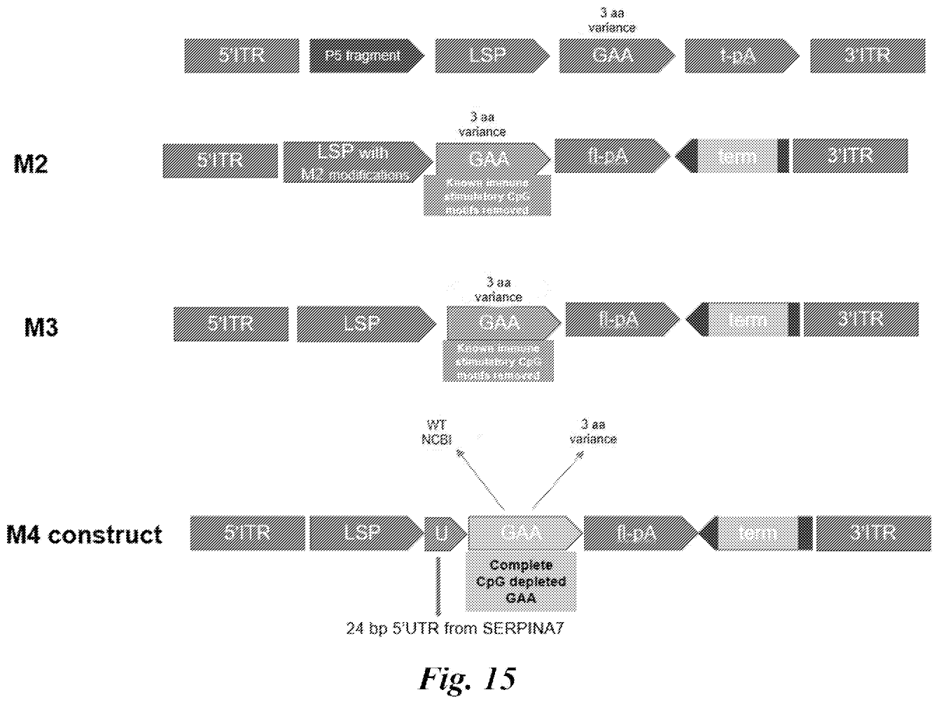

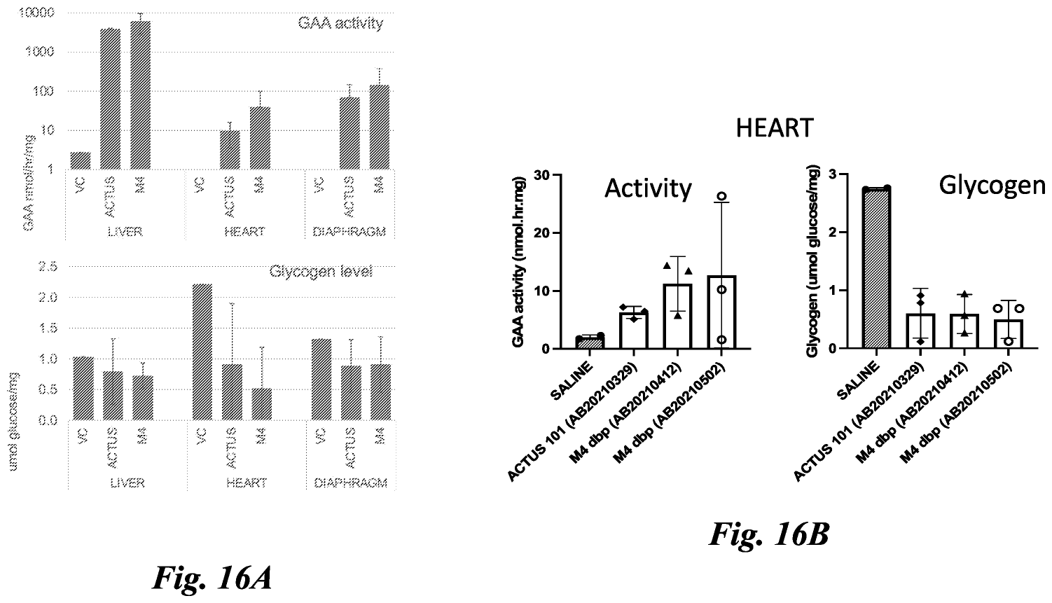

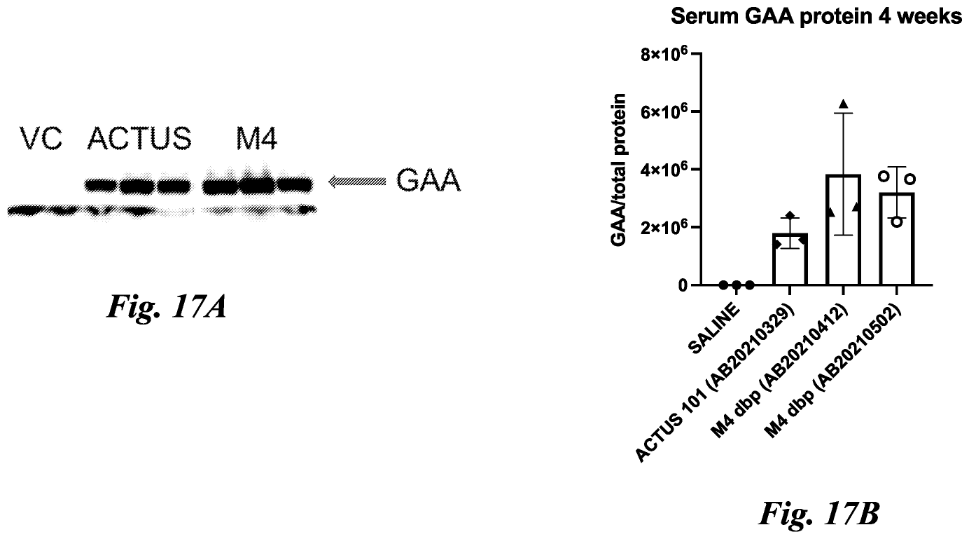

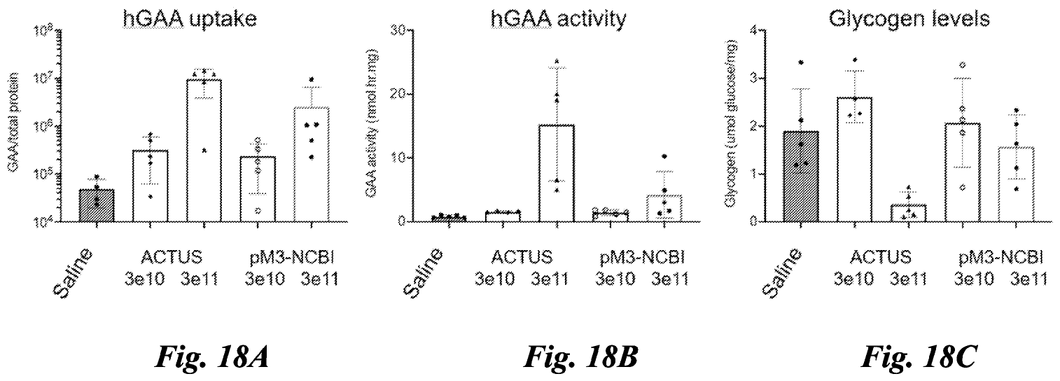

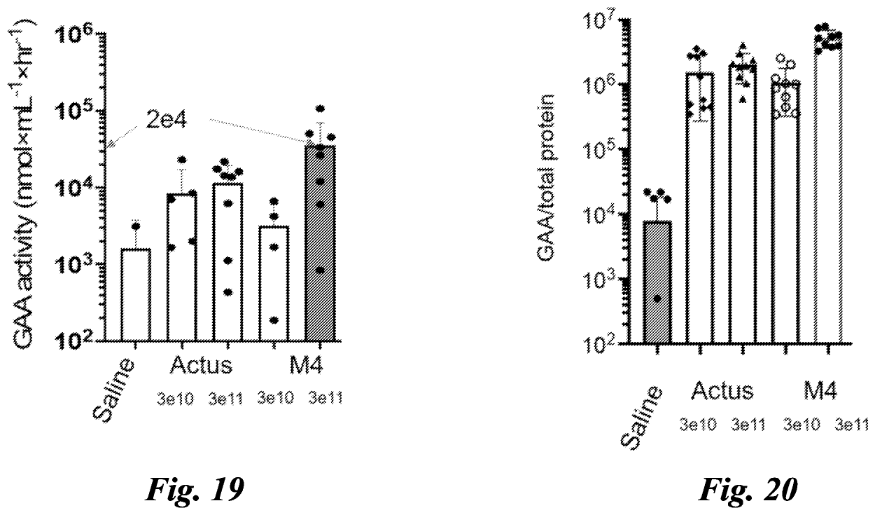

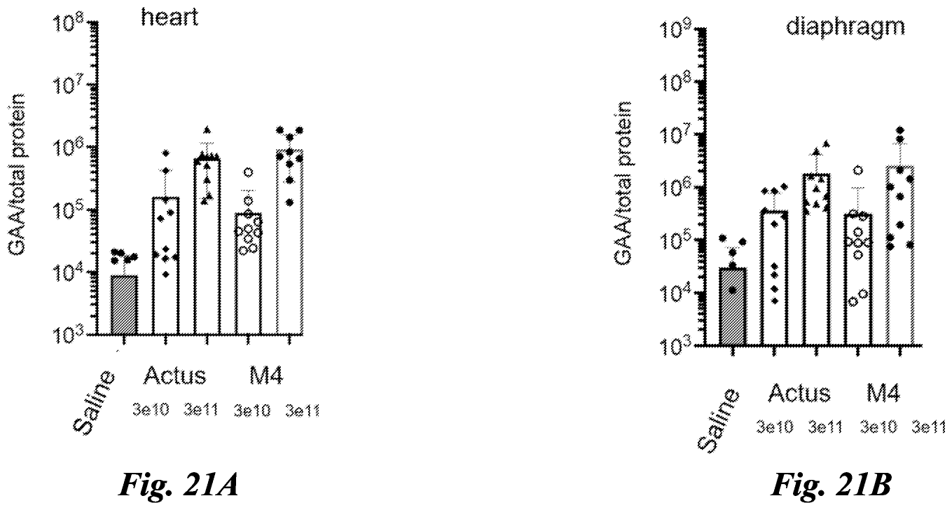

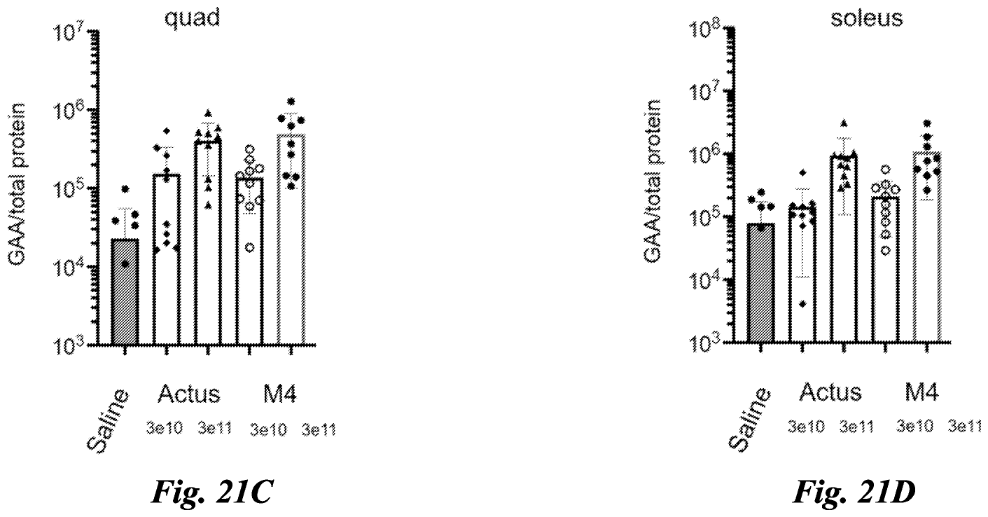

Finally, as shown in FIGS. 14A-14C, a significant dose dependent reduction in glycogen was observed following administration of low and high doses of the M4 construct as compared to Actus 101. This dose dependent reduction was observed in various target tissues, including the heart, diaphragm and liver 8 weeks post administration with either a high or low dose of the M4 construct.

Accordingly, the data presented herein in this Example indicates that the M4 construct out performs Actus 101 with regards to increasing GAA protein levels and activity that can be sustained over a long period of time (e.g., more than 8 weeks) and that was capable of inducing glycogen clearance in the cell.

### Example 4: Comparison of the Various M4 Constructs

Numerous M4 constructs having different codon-optimized GAA sequences have been developed and tested for their ability to induce superior levels of GAA protein and activity, as well as their ability to promote glycogen clearance in the cell. Four M4 constructs, i.e., Seg12 (SEQ ID NO: 30), Seq99 (SEQ ID NO: 29), Seq3 (SEQ ID NO:28), and Seq100 (SEQ ID NO: 27), which comprise codon optimized nucleic acid sequences: SEQ ID NO:7 (Seq12), SEQ ID NO:13, (Seq99), SEQ ID NO: 3 (Seq100) and SEQ ID NO: 4 (Seq3), respectively, which encode a GAA polypeptide of SEQ ID NO: 1, were selected for further study, as being identified as being best in class, and data provided herein this example shows the comparison of these constructs.

All vectors were made at AskBio RTP using proprietary Pro10 cell production system, e.g., as described in U.S. Pat. No. 9,441,206, the contents of which are incorporated herein by reference in its entirety. The constructs were packaged in the AAV8 viral capsid and titered by digital droplet PCR (ddPCR) method using primers directed at vector ITRs.

All assays and analysis were performed as described herein in Example 2.

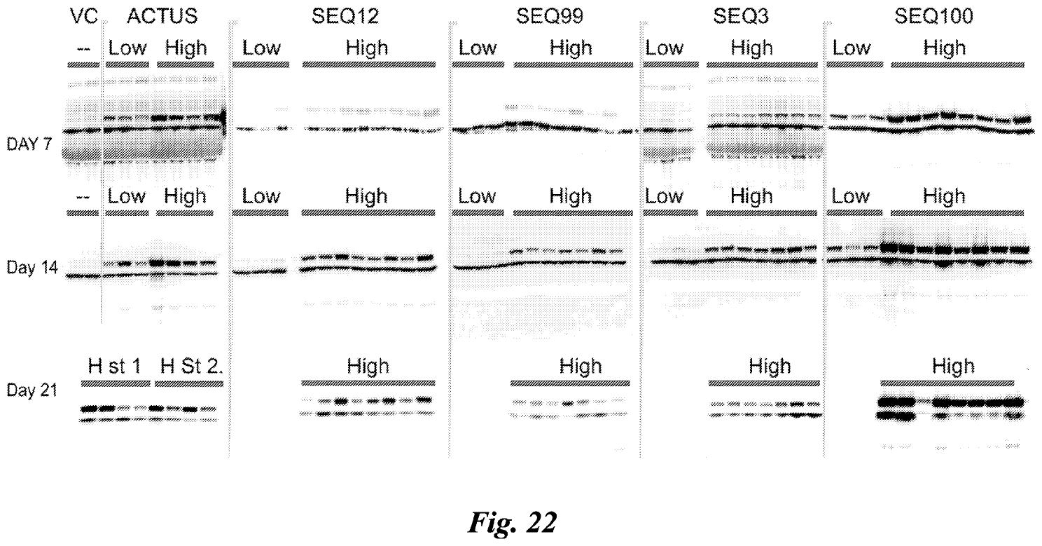

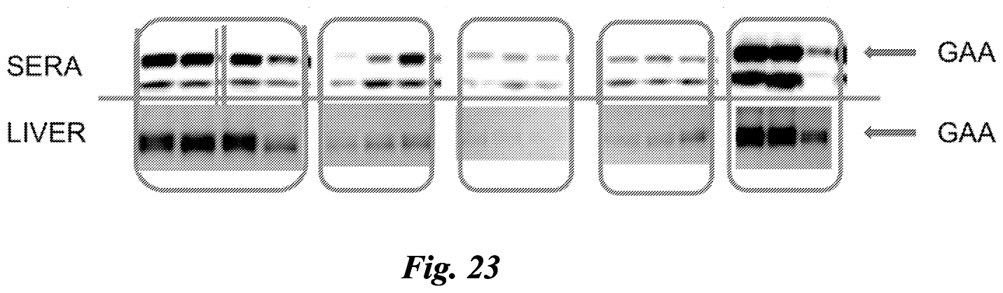

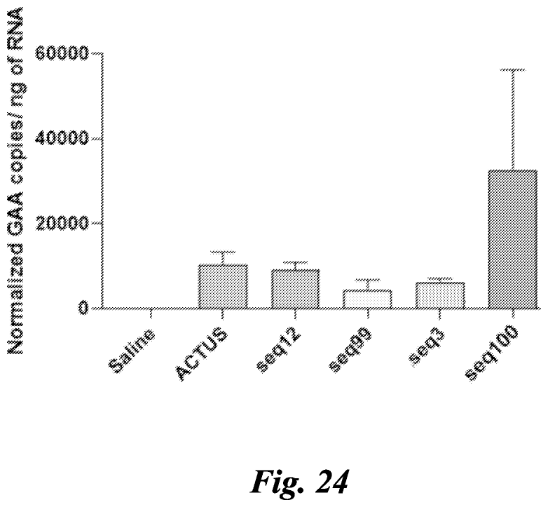

Observations: As shown in FIG. 22, expression of GAA in serum driven from the Seq100 construct was markedly higher at days 7, 14 and 21 post administration as compared to the Actus 101 and the other M4 constructs (i.e., Seq12, Seq99, and Seq3). It was further noted that not all M4 constructs were capable of driving sustainable GAA levels in a similar manner; Seq12, Seq99, Seq3 fail to drive high expression of GAA that was sustained for at least 21 days post administration. Seq100 was additionally found to drive high expression of GAA protein in the liver 21 days post administration, whereas other M4 constructs fail to do so (see FIG. 23). Similarly, increased GAA RNA copy numbers were markedly increased following Seq100 administration as compared to Actus 101 or the other M4 constructs. Accordingly, the data presented herein indicate that Seq100 was a superior M4 construct with regards to driving sustainable, high levels of GAA protein as compared to the other M4 constructs tested.

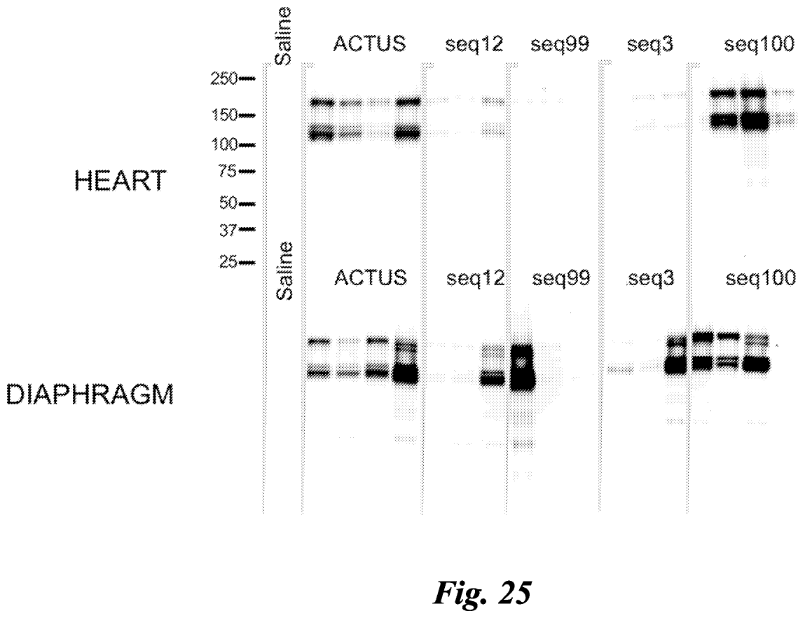

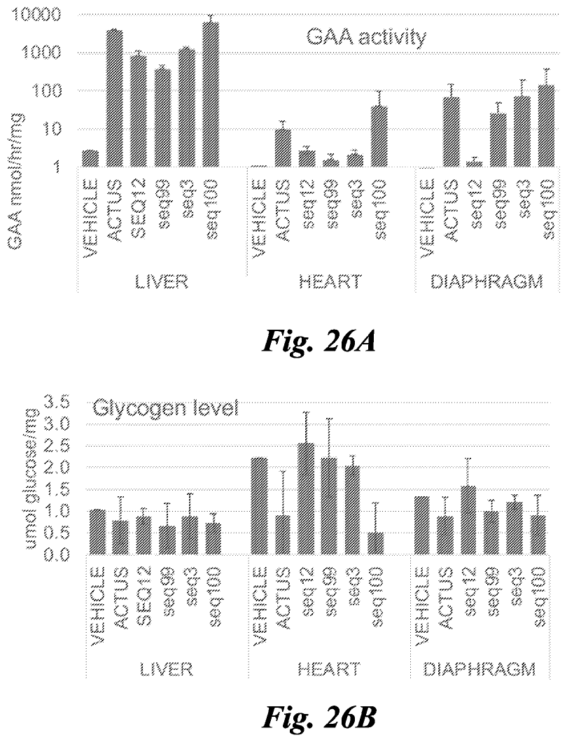

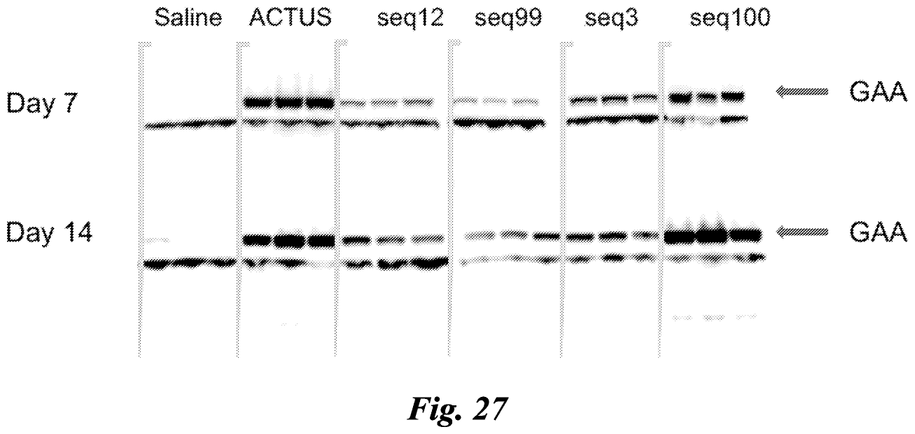

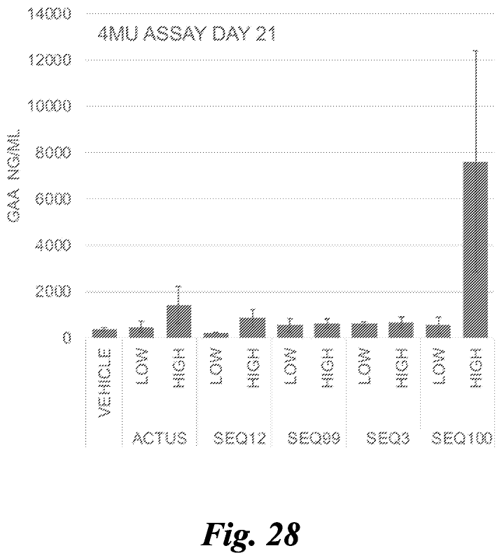

To further assess Seq100's capability to drive high levels of GAA protein expression in target tissues, GAA protein levels and activity, as well as glycogen clearance from the cell was assessed. As shown in FIG. 25, GAA protein levels were markedly higher than Actus or other M4 constructs 21 days post administration in the liver, heart and diaphragm. Increased GAA activity levels were similarly observed in the liver, heart and diaphragm in Seq100-administered mice as compared to Actus or other M4 constructs 21 days post administration (see, e.g., FIG. 26A). Finally, a significant reduction of glycogen was observed in the liver, heart and diaphragm in Seq100-administered mice as compared to Actus or other M4 constructs 21 days post administration (see, e.g., FIG. 26B). Accordingly, the data presented herein indicate that Seq100 (comprising codon optimized nucleic acid sequence of SEQ ID NO: 3) was a superior M4 construct with regards to driving sustainable high levels of GAA protein and activity in target tissues (e.g., liver, heart, and diaphragm) as compared to the other M4 constructs tested.

The data presented herein in this example show that a specific M4 construct, Seq100, was surprisingly, and unexpectedly superior as compared to the other M4 constructs exemplified herein (i.e., Seq12, Seq99, and Seq3). This indicates that the results presented herein were not due to the presence of a codon optimized GAA sequence, but rather, was due to the specific codon optimized sequence included in the Seq100 construct, which is SEQ ID NO: 3.

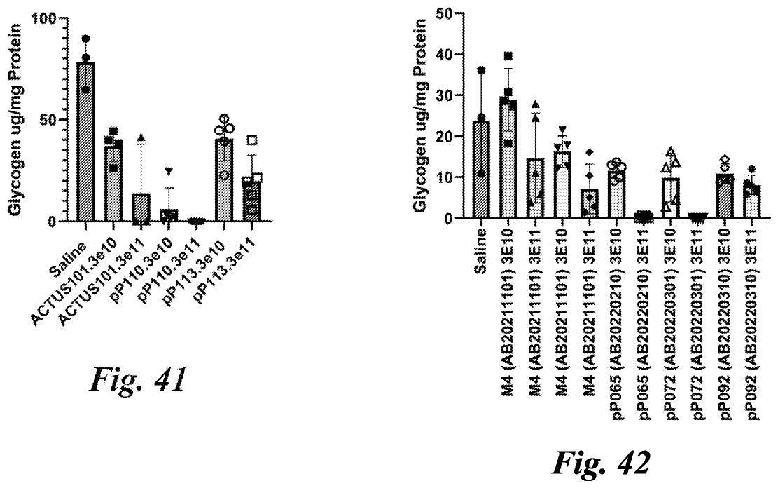

Further, as shown herein in FIG. 42, the M4 variants p065 and p072 showed a greater reduction in glyocogen present in the heart as compared to M4 following administration. This result was found to be dosage dependent; a greater reduction in glyocogen in the heart was observed at a dose of 3E11 as compared to 3E10 for both p065 and p072.

### Example 5: Constructs Encoding a GAA-Signal Peptide, a Heterologous Signal Peptide, and a GAA Polypeptide

Numerous constructs comprising a GAA-signal peptide, or a fragment thereof, a heterologous signal peptide, and a GAA polypeptide were generated. Exemplary constructs generated are described in Table 10 herein. In particular, SEQ ID NO: 463 and 464 are exemplary plasmids (pP112 and pP113, respectively) where pP112, (see, e.g., FIG. 29) is a plasmid comprising in the 5′ to 3′ direction: [ITR]-LSP-[1-27aa GAA-SP]-[201Ip]-[GAA polypeptide starting at amino acid 57 of SEQ ID NO: 1]-[3′UTR]-[hGH polyA]-ITR2, and pP113 (see, e.g., FIG. 29) is a plasmid comprising in a 5′ to 3′ direction: [ITR]-LSP-[1-24aa GAA-SP]-met-[201Ip]-[GAA polypeptide starting at amino acid 57 of SEQ ID NO: 1]-[3′UTR]-[hGH polyA]-ITR2. The nucleic acid encoding the N-terminal truncated GAA polypeptide in pP112 and pP113 can be readily substituted for any nucleic acid sequence encoding a GAA polypeptide, including codon optimized nucleic acid sequences: SEQ ID NO:7 (Seq12), SEQ ID NO:13, (Seq99), SEQ ID NO: 3 (Seq100) and SEQ ID NO: 4 (Seq3), as well as 5′ deletions thereof, such that the nucleic acid sequences encode for a N-terminal truncated GAA polypeptide beginning at amino acid residues selected from any of: 28, 35, 40, 50, 57, 57, 68, 69, 70, 72, 74, 89, 779, 790, 791, 792, 793 or 796. Similarly, the 201Ip (having an amino acid sequence of: MEFGLSWVFLVALLKGVQCE (SEQ ID NO: 60) encoded by nucleic acid sequence SEQ ID NO: 54) is an exemplary heterologous signal peptide included in plasmids pP112 and pP113, and can be readily substituted for other heterologous leader peptide disclosed herein, e.g., any of: wtIL2 lp: MYRMQLLSCIALSLALVTNS (SEQ ID NO: 61) encoded by nucleic acid sequence SEQ ID NO: 55, or mutIL2 lp: MYRMQLLLLIALSLALVTNS (SEQ ID NO: 62) encoded by nucleic acid sequence SEQ ID NO: 56, A2M signal peptide MGKNKLLHPSLVLLLLVLLPTDA (SEQ ID NO: 63) encoded by nucleic acid sequence SEQ ID NO: 57, PZP signal peptide MRKDRLLHLCLVLLLILLSASDSNS (SEQ ID NO: 64) encoded by nucleic acid sequence SEQ ID NO: 58.

rAAV vectors were generated using the Pro10 cell production system, e.g., as described in U.S. Pat. No. 9,441,206, the contents of which are incorporated herein by reference in its entirety. The constructs were packaged in the AAV8 viral capsid and tittered by digital droplet PCR (ddPCR) method using primers directed at vector ITRs. All assays and analysis were performed as described herein in Example 3.

Analysis and Observations: pP112 and pP113 were used to generate infectious non-replicating recombinant adeno-associated viral vector (AAV) serotype 8, pseudotyped with AAV2 inverted terminal repeats (ITR), expressing human GAA under the control of a liver specific promoter (LSP), where there is a GAA-signal peptide (or 1-24 amino acid portion of the GAA-signal peptide and a heterologous 201lp signal peptide to promote expression of the GAA polypeptide, and where the GAA polypeptide is a N-terminal truncated protein beginning at amino acid 57 of SEQ ID NO:1.

Male GAA knock-out mice were injected with the generated AAV via a tail vein injection; 5 male mice were included in each group (i.e., control (saline), pP112, pP113). AAV was administered at a dose of 1×10⁶ in a total volume of 150 ul. Weekly bleeds were performed on the mice for 4 weeks. Each mouse was then sacrificed at 4 weeks, and serum and target tissue was removed for anaylsis.

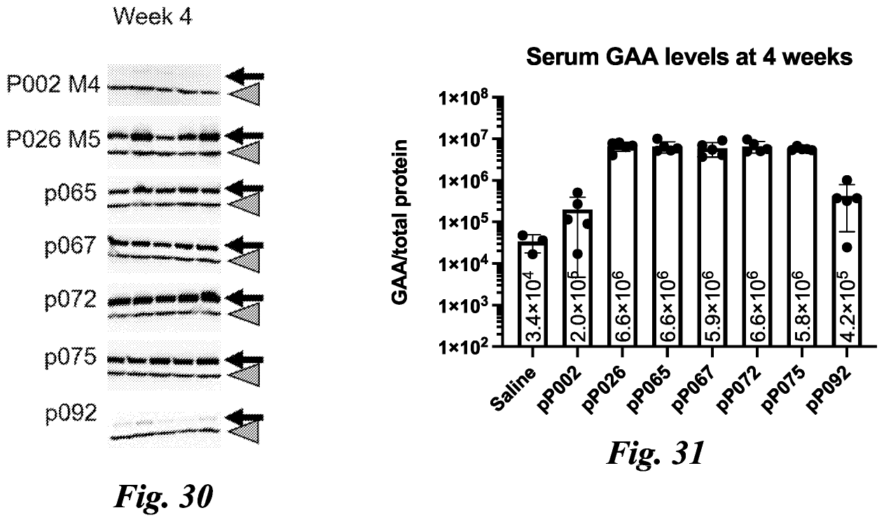

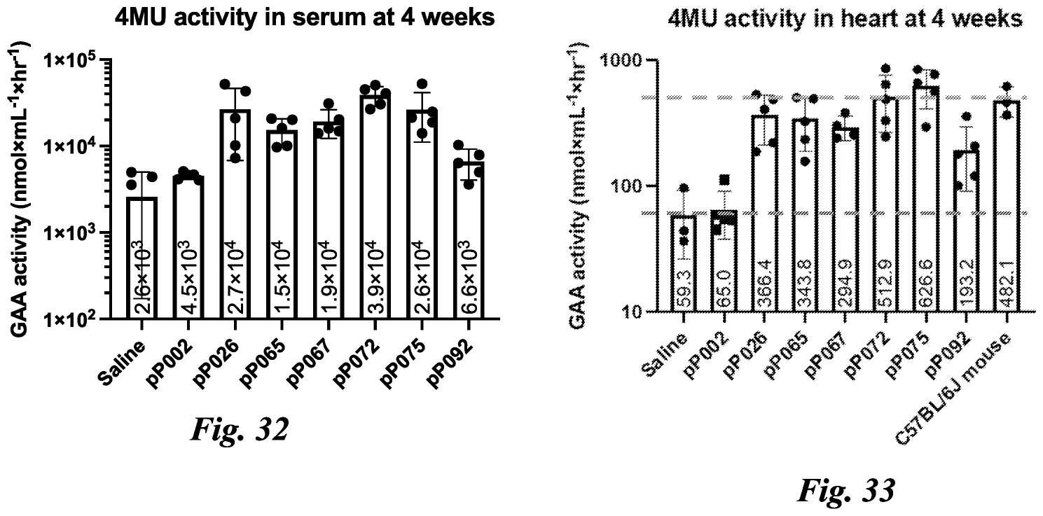

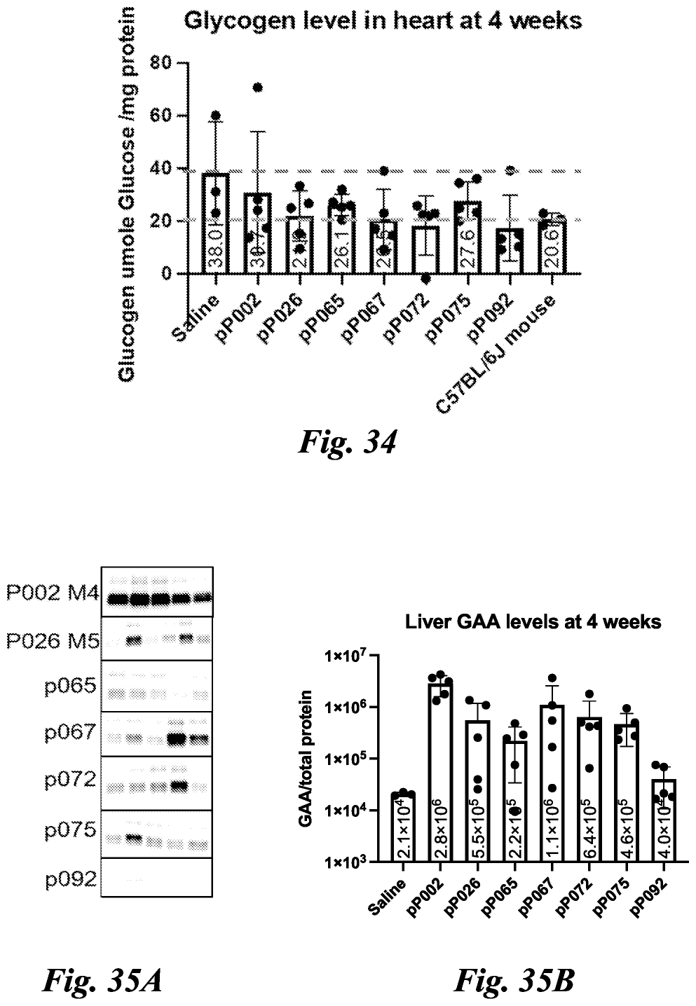

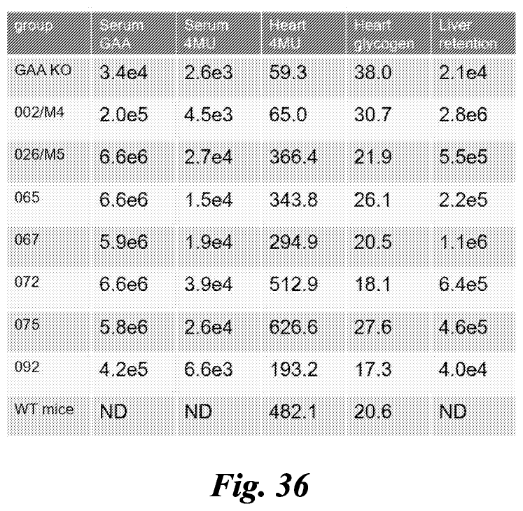

Expression of GAA in serum was assessed 4 weeks post administration of rAAV generated from pP112 and pP113 plasmids. After 4 weeks, the animals were sacrificed and tissue was harvested. GAA expression was assessed in sera over time by western blotting (FIG. 30) and semi-quantitative densitometry (FIG. 31). GAA uptake by the liver was also assessed by western blotting (FIG. 35A) and semi-quantitative densitometry (FIG. 35B). GAA enzymatic activity in sera, and heart over time was assessed by 4MU assay (FIGS. 32 and 33). Glycogen content was also assessed in the heart (FIG. 34), as described in Example 3.

rAAV encoding GAA polypeptide comprising both a N-terminal endogenous GAA signal peptide or a fragment thereof, and a heterologous 201lp signal peptide promoted GAA expression in the serum (FIGS. 30 and 31), and increased GAA activity in the serum and target tissues (e.g., heart) (FIGS. 32 and 33). A reduction of glycogen levels in the heart as compared a rAAV encoding a GAA polypeptide comprising only a N-terminal heterologous signal peptide was also observed (FIG. 34). Finally, a reduction in GAA retention in the liver was noted 4 weeks following administration of the rAAV (FIGS. 35A and 35B).

### Example 6: Constructs Encoding a GAA-Signal Peptide, a Heterologous Signal Peptide, and a GAA Polypeptide

pP112 and pP113 are used to generate infectious non-replicating recombinant adeno-associated viral vector (AAV) serotype 8, pseudotyped with AAV2 inverted terminal repeats (ITR), expressing human GAA under the control of a liver specific promoter (LSP), where there is a GAA-signal peptide (or 1-24 amino acid portion of the GAA-signal peptide and a heterologous 201lp signal peptide to promote expression of the GAA polypeptide, and where the GAA polypeptide is a N-terminal truncated protein beginning at amino acid 57 of SEQ ID NO:1, as described in Example 5.

Male GAA knock-out mice are injected with the generated AAV via a tail vein injection; 5 male mice are included in each group (i.e., control (saline), pP112, pP113), as described in Example 5. AAV is administered at a dose of 1×10⁶ in a total volume of 150 ul. Weekly bleeds are performed on the mice for 12 weeks. Each mouse is then sacrificed at 12 weeks, and serum and target tissue is removed for anaylsis.

Expression of GAA in serum is assessed 12-weeks after administration of rAAV generated from pP112 and pP113 plasmids. After 12 weeks, the animals are sacrificed and tissue harvested, and GAA expression in sera over time by western blotting and semi-quantitative densitometry is assessed, as well as GAA uptake by target organs (heart, diaphragm) by western blotting and semi-quantitative densitometry. GAA enzymatic activity in sera over time is assessed by 4MU assay; and GAA enzymatic activity in liver is assessed by 4MU assay; GAA uptake by select tissues (heart, diaphragm) is assessed by 4MU assays; glycogen content in select tissues (heart, diaphragm) is also assessed, as described in Example 3.

rAAV encoding GAA polypeptide comprising both a N-terminal endogenous GAA signal peptide or a fragment thereof, and a heterologous 201lp signal peptide promotes GAA expression in the serum, and increases GAA activity in the serum and target tissues (e.g., heart). A reduction of glycogen levels in the heart as compared a rAAV encoding a GAA polypeptide comprising only a N-terminal heterologous signal peptide is also observed. Finally, a reduction in GAA retention in the liver is noted 12 weeks following administration of the rAAV with p113 compared to p112.

### Example 7: Constructs Encoding a GAA-Signal Peptide, a Heterologous Signal Peptide, and a GAA Polypeptide

Numerous constructs comprising a GAA-signal peptide, or a fragment thereof, a heterologous signal peptide, and a GAA polypeptide, are modified to include an optimized liver-specific promoter. Exemplary constructs that are modified are described in Table 10 herein. For example, the liver-specific promoter (LSP) described in SEQ ID NO: 463 and 464 is swapped with the SP0412 promoter (SEQ ID NO: 91) to enhance the expression of the construct in the liver. For example, such modified exemplary plasmids sequences would include a plasmid comprising in the 5′ to 3′ direction: [ITR]-SP0412-[1-27aa GAA-SP]-[201Ip]-[GAA polypeptide starting at amino acid 57 of SEQ ID NO: 1]-[3′UTR]-[hGH polyA]-ITR2 (i.e., a modified pP112), and a plasmid comprising in a 5′ to 3′ direction: [ITR]-SP0412-[1-24aa GAA-SP]-met-[201Ip]-[GAA polypeptide starting at amino acid 57 of SEQ ID NO: 1]-[3′UTR]-[hGH polyA]-ITR2 (i.e., a modified pP113). The nucleic acid encoding the N-terminal truncated GAA polypeptide in pP112 and pP113 can be readily substituted for any nucleic acid sequence encoding a GAA polypeptide, including codon optimized nucleic acid sequences: SEQ ID NO:7 (Seq12), SEQ ID NO:13, (Seq99), SEQ ID NO: 3 (Seq100) and SEQ ID NO: 4 (Seq3), as well as 5′ deletions thereof, such that the nucleic acid sequences encode for a N-terminal truncated GAA polypeptide beginning at amino acid residues selected from any of: 28, 35, 40, 50, 57, 57, 68, 69, 70, 72, 74, 89, 779, 790, 791, 792, 793 or 796. Similarly, the 201Ip (having an amino acid sequence of: MEFGLSWVFLVALLKGVQCE (SEQ ID NO: 60) encoded by nucleic acid sequence SEQ ID NO: 54) is an exemplary heterologous signal peptide included in plasmids pP112 and pP113, and can be readily substituted for other heterologous leader peptide disclosed herein, e.g., any of: wtIL2 lp: MYRMQLLSCIALSLALVTNS (SEQ ID NO: 61) encoded by nucleic acid sequence SEQ ID NO: 55, or mutIL2 lp: MYRMQLLLLIALSLALVTNS (SEQ ID NO: 62) encoded by nucleic acid sequence SEQ ID NO: 56, A2M signal peptide MGKNKLLHPSLVLLLLVLLPTDA (SEQ ID NO: 63) encoded by nucleic acid sequence SEQ ID NO: 57, PZP signal peptide MRKDRLLHLCLVLLLILLSASDSNS (SEQ ID NO: 64) encoded by nucleic acid sequence SEQ ID NO: 58.

Modified plasmids described in this Example are used to generate infectious non-replicating recombinant adeno-associated viral vector (AAV) serotype 8, pseudotyped with AAV2 inverted terminal repeats (ITR), expressing human GAA under the control of the SP0412 promoter, where there is a GAA-signal peptide (or 1-24 amino acid portion of the GAA-signal peptide and a heterologous 201lp signal peptide to promote expression of the GAA polypeptide, and where the GAA polypeptide is a N-terminal truncated protein beginning at amino acid 57 of SEQ ID NO:1, as described in Example 5.

Male GAA knock-out mice are injected with the generated AAV via a tail vein injection as described in Example 5. Expression of GAA in serum is assessed 4 weeks after administration of rAAV generated from modified plasmids. After 4 weeks, the animals are sacrificed and tissue harvested, and GAA expression in sera over time by western blotting and semi-quantitative densitometry is assessed, as well as GAA uptake by target organs (heart, diaphragm) by western blotting and semi-quantitative densitometry. GAA enzymatic activity in sera over time is assessed by 4MU assay; and GAA enzymatic activity in liver is assessed by 4MU assay; GAA uptake by select tissues (heart, diaphragm) is assessed by 4MU assays; glycogen content in select tissues (heart, diaphragm) is also assessed, as described in Example 3.

rAAV encoding GAA polypeptide comprising both a N-terminal endogenous GAA signal peptide or a fragment thereof, and a heterologous 201lp signal peptide promotes GAA expression in the serum, and increases GAA activity in the serum and target tissues (e.g., heart). A reduction of glycogen levels in the heart as compared a rAAV encoding a GAA polypeptide comprising only a N-terminal heterologous signal peptide is also observed. Finally, a reduction in GAA retention in the liver is noted 4 weeks following administration of the rAAV with modified p113 compared to p112.

### Example 8: Constructs Encoding a 5′UTR with Intron, GAA-Signal Peptide, a Heterologous Signal Peptide, and a GAA Polypeptide

Numerous constructs comprising a fragment of the GAA-signal peptide, a heterologous signal peptide, and a GAA polypeptide are modified to include a 5′UTR+intron sequence upstream of the GAA sequence. In particular, constructs P072 and P092 (see, e.g., Table 10) are modified to include three changes, generating mod-P072 and mod-P092, respectively.

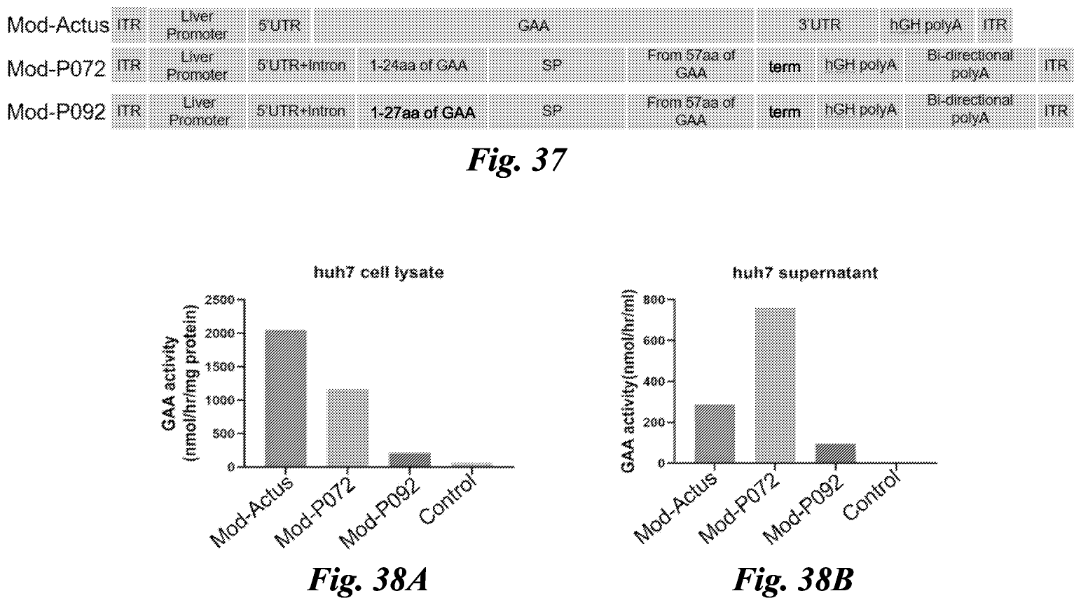

First, the 5′UTR of P072 and P092 is replaced with a 5′UTR comprising an intron sequence. Second, the 3′UTR of P072 and P092 is removed. And third, a bi-directional polyA sequence is added immediately following the hGH polyA sequence of P072 and P092. Accordingly, mod-P072 is a construct comprising in the 5′ to 3′ direction: [ITR]-LSP-[5′UTR+intron]-[1-24aa GAA-SP]-[Signal Peptide]-[GAA polypeptide starting at amino acid 57 of SEQ ID NO: 1]-[hGH polyA]-[Bi-directional polyA]-[ITR2]. Mod-P092 is a construct comprising in the 5′ to 3′ direction: [ITR]-LSP-[5′UTR+intron]-[1-27aa GAA-SP]-[Signal Peptide]-[GAA polypeptide starting at amino acid 57 of SEQ ID NO: 1]-[hGH polyA]-[Bi-directional polyA]-[ITR2](FIG. 37)

Further, the Actus construct is modified to remove residual wtAAV DNA sequences immediately 5′ and 3′ to the ITRs, generating the mod-Actus construct. Such removal of residual wtAAV DNA avoids homologous recombination between the Rep and Cap plasmid used in generating rcAAV. mod-P072 and mod-P092 constructs are also modified to remove the residual wtAAV DNA immediately 5′ and 3′ to the ITRs. (FIG. 37)

Modified plasmids described in this Example, i.e., mod-Actus, mod-P072 and mod-P092, are used to generate infectious non-replicating recombinant adeno-associated viral vector (AAV) serotype 8, pseudotyped with AAV2 inverted terminal repeats (ITR), expressing human GAA under the control of the LSP promoter, where there is a GAA-signal peptide (or a 1-24 amino acid or a 1-27 amino acid portion of the GAA-signal peptide) and a heterologous signal peptide to promote expression of the GAA polypeptide, and where the GAA polypeptide is a N-terminal truncated protein beginning at amino acid 57 of SEQ ID NO:1, as described in Example 5.

The generated AAV viruses are used to infect Huh7 cells to assess their efficacy. 6-well culture dishes comprising a population of seeded Huh7 cells are infected at an MOI of 1E⁶ vg/cell. 48 hours post infection, cell lysates and cell supernatants are obtained from the culture. GAA enzymatic activity is measured in the cell lysate (i.e., nmol/hr/mg protein) and supernatant (i.e., nmol/hr/ml). AAV viruses generated from the mod-Actus construct display the highest GAA enzymatic activity in the cell, and AAV viruses generated from the mod-P072 construct yield the highest level of GAA secretion into the supernatant (FIGS. 38A and 38B).

To assess the efficacy of the AAVs in vivo, male and female wild-type mice (C57BL/6J) are injected with the generated AAV via a tail vein injection. Each virus is administered at a low dose (3E¹⁰), mid-level dose (6E¹⁰), and high dose (3E¹¹) per mouse in a 150 μl total volume. GAA expression levels and activity are assessed in sera weekly for 4 weeks following administration. After 4 weeks, the animals are sacrificed and tissue harvested, and GAA expression in sera over time by western blotting and semi-quantitative densitometry is assessed, as well as GAA uptake by target organs (heart, quadriceps muscle) by western blotting and semi-quantitative densitometry. GAA enzymatic activity in sera over time is assessed by 4MU assay; GAA enzymatic activity in liver is assessed by 4MU assay; and GAA uptake by select tissues (heart, quadriceps muscle) is assessed by 4MU assays.

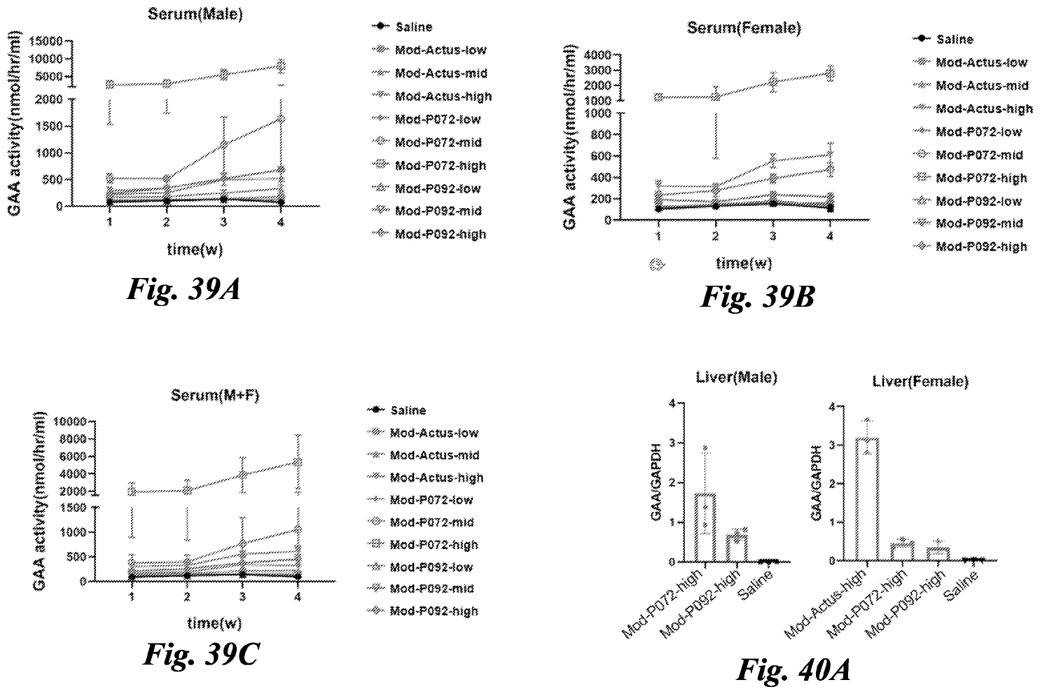

Consistent with the in vitro data, rAAVs expressing the 1-24 amino acid portion of the GAA-signal peptide, a 5′UTR+intron, and a bi-directional polyA (i.e., generated by the mod-P072 construct) exhibit the highest level of GAA in the serum of male and female mice (FIG. 39A-39C).

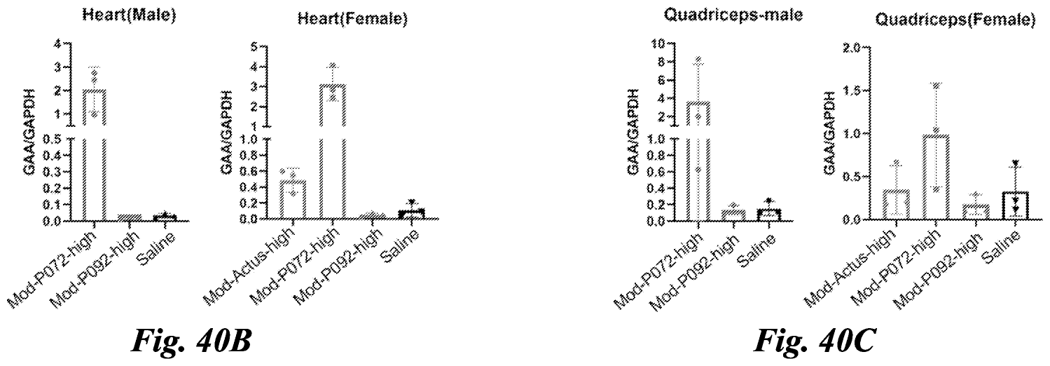

To assess what tissues express the modified rAAVs described herein in this example, liver, heart, and quadriceps tissue are assessed for GAA levels following administration. rAAVs expressing the full length GAA signal peptide (i.e., generated by the mod-Actus construct) exhibit the highest expression level of GAA in the liver in vivo at a high dose (FIG. 40A), whereas rAAVs expressing the 1-24 amino acid portion of the GAA-signal peptide, a 5′UTR+intron, and a bi-directional polyA (i.e., generated by the mod-P072 construct) exhibits the highest level of GAA uptake in the heart and quadriceps muscle at a high dose (FIGS. 40B and 40C).

Further mod-Actus AAV exhibits the highest expression level of GAA in the liver at low and mid-level doses, as compared to mod-P072 and mod-P092 AAVs, indicating that GAA is retained in the liver at a higher rate. Mod-P072 AAV exhibits a higher GAA activity in the heart, as compared to mod-Actus and mod-P092, indicating that mod-P072 AAV has a greater uptake in the heart. Mod-Actus and mod-P072 AAVs exhibit a higher GAA activity in the quadriceps muscle as, compared to mod-P092.

### REFERENCES

Champiat S, Raposo RAS, Maness N J, Lehman J L, Purtell S E, Hasenkrug A M, et al. (2012) Influence of HAART on Alternative Reading Frame Immune Responses over the Course of HIV-1 Infection. PLoS ONE 7(6): e39311. https://doi.org/10.1371/journal.pone.0039311
Faust S M, Bell P, Cutler B J, et al. CpG-depleted adeno-associated virus vectors evade immune detection. J Clin Invest. 2013; 123(7):2994-3001. doi:10.1172/JCI68205
Han S O, Ronzitti G, Arnson B, et al. Low-Dose Liver-Targeted Gene Therapy for Pompe Disease Enhances Therapeutic Efficacy of ERT via Immune Tolerance Induction [published correction appears in Mol Ther Methods Clin Dev. 2019 May 1; 13:431]. Mol Ther Methods Clin Dev. 2017; 4:126-136. Published 2017 Jan. 11. doi:10.1016/j.omtm.2016.12.010
Han S O, Li S, McCall A, et al. Comparisons of Infant and Adult Mice Reveal Age Effects for Liver Depot Gene Therapy in Pompe Disease. Mol Ther Methods Clin Dev. 2019; 17:133-142. Published 2019 Dec. 6. doi:10.1016/j.omtm.2019.11.020
Kracht, M., van Lummel, M., Nikolic, T. et al. Autoimmunity against a defective ribosomal insulin gene product in type 1 diabetes. Nat Med 23, 501-507 (2017). https://doi.org/10.1038/nm.428
Krieg A M. CpG motifs in bacterial DNA and their immune effects. Annu Rev Immunol. 2002; 20:709-760. doi:10.1146/annurev.immunol.20.100301.064842
Lorenz F K, Wilde S, Voigt K, et al. Codon optimization of the human papillomavirus E7 oncogene induces a CD8+T cell response to a cryptic epitope not harbored by wild-type E7. PLoS One. 2015; 10(3):e0121633. Published 2015 Mar. 23. doi:10.1371/journal.pone.0121633
Schirmbeck R, Riedl P, Fissolo N, Lemonnier F A, Bertoletti A, Reimann J. Translation from cryptic reading frames of DNA vaccines generates an extended repertoire of immunogenic, MHC class I-restricted epitopes. J Immunol. 2005; 174(8):4647-4656. doi:10.4049/jimmunol.174.8.4647
Tóth R, Mészáros I, Hüser D, et al. Methylation Status of the Adeno-Associated Virus Type 2 (AAV2). Viruses. 2019; 11(1):38. Published 2019 Jan. 9. doi:10.3390/v11010038
Yewdell J W, Hickman H D. New lane in the information highway: alternative reading frame peptides elicit T cells with potent antiretrovirus activity. J Exp Med. 2007; 204(11):2501-2504. doi:10.1084/jem.20071986
   Amalfitano, A., et al., 2001. ‘Recombinant human acid alpha-glucosidase enzyme therapy for infantile glycogen storage disease type II: results of a phase I/II clinical trial’, Genet Med, 3: 132-8.
AmericanThoracicSociety. 1991. ‘Lung function testing: selection of reference values and interpretative strategies. American Thoracic Society’, Am Rev Respir Dis, 144: 1202-18.
Angelini, C., et al., 2009. ‘Progress in Enzyme Replacement Therapy in Glycogen Storage Disease Type II’, Ther Adv Neurol Disord, 2: 143-53.
‘ATS statement: guidelines for the six-minute walk test’. 2002. Am J Respir Crit Care Med, 166: 111-7.
Banugaria, S. G., et al., 2011. ‘The impact of antibodies on clinical outcomes in diseases treated with therapeutic protein: lessons learned from infantile Pompe disease’, Genet Med, 13: 729-36.
Brinster, R. L., et al., 1988. ‘Introns increase transcriptional efficiency in transgenic mice’, Proc Natl Acad Sci USA, 85: 836-40.
Brooke, M. H., et al., 1983. ‘Clinical investigation in Duchenne dystrophy: 2. Determination of the “power” of therapeutic trials based on the natural history’, Muscle Nerve, 6: 91-103.
Brooke, M. H., et al., 1987. ‘Clinical investigation of Duchenne muscular dystrophy. Interesting results in a trial of prednisone’, Arch Neurol, 44: 812-7.
Carlier, R. Y., et al., 2011. ‘Whole-body muscle MRI in 20 patients suffering from late onset Pompe disease: Involvement patterns’, Neuromuscul Disord, 21: 791-9.
Crudele, J. M., et al., 2015. ‘AAV liver expression of FIX-Padua prevents and eradicates FIX inhibitor without increasing thrombogenicity in hemophilia B dogs and mice’, Blood, 125: 1553-61.
Daya, S., and K. I. Berns. 2008. ‘Gene therapy using adeno-associated virus vectors’, Clin Microbiol Rev, 21: 583-93.
de Vries, J. M., et al., 2017. ‘Pompe disease in adulthood: effects of antibody formation on enzyme replacement therapy’, Genet Med, 19: 90-97.
Desnick, R. J. 2004. ‘Enzyme replacement and enhancement therapies for lysosomal diseases’, J Inherit Metab Dis, 27: 385-410.
Duan, D. 2018. ‘Systemic AAV micro-dystrophin gene therapy for Duchenne Muscular Dystrophy’, Mol Ther, 26: 2337-56.
El-Gharbawy, A. H., et al., 2011. ‘Expanding the clinical spectrum of late-onset Pompe disease: dilated arteriopathy involving the thoracic aorta, a novel vascular phenotype uncovered’, Mol Genet Metab, 103: 362-6.
Flotte, T. R., et al., 2004. ‘Phase I trial of intramuscular injection of a recombinant adeno-associated virus alpha 1-antitrypsin (rAAV2-CB-hAAT) gene vector to AAT-deficient adults’, Hum Gene Ther, 15: 93-128.
Franco, L. M., et al., 2005. ‘Evasion of immune responses to introduced human acid alpha-glucosidase by liver-restricted expression in glycogen storage disease type II’, Mol Ther, 12: 876-84.
Han, S. O., G et al., 2017. ‘Low-Dose Liver-Targeted Gene Therapy for Pompe Disease Enhances Therapeutic Efficacy of ERT via Immune Tolerance Induction’, Mol Ther Methods Clin Dev, 4: 126-36.
Hahn, S. H., et al., 2018. ‘Efficacy, safety profile, and immunogenicity of alglucosidase alfa produced at the 4,000-liter scale in U S children and adolescents with Pompe disease: ADVANCE, a phase IV, open-label, prospective study’, Genet Med, 20: 1284-94.
Hobson-Webb, L. D., et al., 2012. ‘Autopsy findings in late-onset Pompe disease: a case report and systematic review of the literature’, Mol Genet Metab, 106: 462-9.
Hundsberger T, Rosler K M, and Findling O. 2014. Cessation and resuming of alglucosidase alfa in Pompe disease: a retrospective analysis. J Neurol. September; 261(9):1684-90.
Ill, C. R., et al., 1997. ‘Optimization of the human factor VIII complementary DNA expression plasmid for gene therapy of hemophilia A’, Blood Coagul Fibrinolysis, 8 Suppl 2: S23-30.
Kishnani, P. S., et al., 2007. ‘Recombinant human acid [alpha]-glucosidase: major clinical benefits in infantile-onset Pompe disease’, Neurology, 68: 99-109.
Kishnani, P. S., et al., 2010. ‘Cross-reactive immunologic material status affects treatment outcomes in Pompe disease infants’, Mol Genet Metab, 99: 26-33.
Koeberl, D. D., et al., 2018. ‘Correction of Biochemical Abnormalities and Improved Muscle Function in a Phase I/II Clinical Trial of Clenbuterol in Pompe Disease’, Mol Ther.
Lachmann, R., and B. Schoser. 2013. ‘The clinical relevance of outcomes used in late-onset Pompe disease: can we do better?’, Orphanet J Rare Dis, 8: 160.
“LUMIZYME Prescribing Information.” In. 2014.
Maga, J. A., et. al., 2013. Glycosylation-independent lysosomal targeting of acid alfa-glucosidase enhances muscle glycogen clearance in pompe mice. J. Biol. Chem. 18 (288): 1428-1438.
Manno, C. S., et al., 2006. ‘Successful transduction of liver in hemophilia by AAV-Factor IX and limitations imposed by the host immune response’, Nat Med, 12: 342-7.
Markusic, D. M., et al., 2013. ‘Effective gene therapy for haemophilic mice with pathogenic factor IX antibodies’, EMBO Mol Med, 5: 1698-709.
Mazaffer, T. 2018. “Updated results from ATB200-02: A first-in-human, open-label, phase ½ study of ATB200 co-administered with AT2221 in adults with Pompe disease.” In 14th annual WORLD symposium.
Mendell, J. R., et al., 2017. ‘Single-Dose Gene-Replacement Therapy for Spinal Muscular Atrophy’, N Engl J Med, 377: 1713-22.
Mendell, J. R. et al., 2016. “Gene therapy for spinal muscular atrophy type 1 shows potential to improve survival and motor funtional outcomes.” In ASGCT; 19th Annual Meeting., S190.
Mendelsohn, N. J., Y. H. Messinger, A. S. Rosenberg, and P. S. Kishnani. 2009. ‘Elimination of antibodies to recombinant enzyme in Pompe's disease’, N Engl J Med, 360: 194-5.
Moxley, R. T., 3rd. 1990. ‘Functional testing’, Muscle Nerve, 13 Suppl: S26-9.
Nathwani, A. C., et al., 2014. ‘Long-term safety and efficacy of factor IX gene therapy in hemophilia B’, N Engl J Med, 371: 1994-2004.
Nathwani, A. C., C et al., 2011. ‘Long-term safety and efficacy following systemic administration of a self-complementary AAV vector encoding human FIX pseudotyped with serotype 5 and 8 capsid proteins’, Mol Ther, 19: 876-85.
Nathwani, A. C., et al., 2011. ‘Adenovirus-associated virus vector-mediated gene transfer in hemophilia B’, N Engl J Med, 365: 2357-65.
Niemeyer, G. P., et al., 2009. ‘Long-term correction of inhibitor-prone hemophilia B dogs treated with liver-directed AAV2-mediated factor IX gene therapy’, Blood, 113: 797-806.
Patel, T. T., S. G. Banugaria, L. E. Case, S. Wenninger, B. Schoser, and P. S. Kishnani. 2012. ‘The impact of antibodies in late-onset Pompe disease: a case series and literature review’, Mol Genet Metab, 106: 301-9.
Ponder, K. P. 2011. ‘Merry christmas for patients with hemophilia B’, N Engl J Med, 365: 2424-5.
Raben, N., et al., 2003. ‘Enzyme replacement therapy in the mouse model of Pompe disease’, Mol Genet Metab, 80: 159-69.
Raj, D., A. M. Davidoff, and A. C. Nathwani. 2011. ‘Self-complementary adeno-associated viral vectors for gene therapy of hemophilia B: progress and challenges’, Expert Rev Hematol, 4: 539-49.
Rangarajan, S., et al., 2017. ‘AAV5-Factor VIII Gene Transfer in Severe Hemophilia A’, N Engl J Med, 377: 2519-30.
Russell, D. J., et al., 1989. The gross motor function measure: a means to evaluate the effects of physical therapy. Dev. Med. Child. Neurol 31: 341-352.
Scheidegger O, et al., 2018. 36-Months follow-up assessment after cessation and resuming of enzyme replacement therapy in late onset Pompe disease: data from the Swiss Pompe Registry. J Neurol. 265 (12):2783-2788. doi: 10.1007/s00415-018-9065-7.
Schoser, B., A. et al., 2017. ‘Survival and long-term outcomes in late-onset Pompe disease following alglucosidase alfa treatment: a systematic review and meta-analysis’, J Neurol, 264: 621-30.
Schwarts, C. E., V. E. Powell, and A. Eldar-Lissai. 2017. ‘Measuring hemophilia caregiver burden: validation of the Hemophilia Caregiver Impact measure’, Qual Life Res, 26: 2551-62.
“Summary of clinical trials using AAV gene transfer.” In. 2015.
Sun, B., et al., 2007. ‘Enhanced response to enzyme replacement therapy in Pompe disease after the induction of immune tolerance’, Am J Hum Genet, 81: 1042-9.
Sun, B., et al., 2010. ‘Immunomodulatory gene therapy prevents antibody formation and lethal hypersensitivity reactions in murine pompe disease’, Mol Ther, 18: 353-60.
Sun, B., et al., 2006. ‘Enhanced efficacy of an AAV vector encoding chimeric, highly secreted acid alpha-glucosidase in glycogen storage disease type II’, Mol Ther, 14: 822-30.
Tarnopolsky, M., ey al., 2016. ‘Pompe Disease: Diagnosis and Management. Evidence-Based Guidelines from a Canadian Expert Panel’, Can J Neurol Sci, 43: 472-85.
van Capelle, C. I., N. A. et al., 2012. ‘The quick motor function test: a new tool to rate clinical severity and motor function in Pompe patients’, J Inherit Metab Dis, 35: 317-23.
van Capelle, C. I., N. A. et al., 2010. ‘Effect of enzyme therapy in juvenile patients with Pompe disease: a three-year open-label study’, Neuromuscul Disord, 20: 775-82.
van der Beek, N. A., C. I. et al., 2011. ‘Rate of progression and predictive factors for pulmonary outcome in children and adults with Pompe disease’, Mol Genet Metab, 104: 129-36.
van der Ploeg, A. T., et al., 2010. ‘A randomized study of alglucosidase alfa in late-onset Pompe's disease’, N Engl J Med, 362: 1396-406.
Vercauteren, K., et al., 2016. ‘Superior in vivo transduction of human hepatocytes using engineered AAV3 capsid.’, Mol Ther, 24: 1042-49.
Ziegler, R. J., et al., 2008. ‘Ability of adeno-associated virus serotype 8-mediated hepatic expression of acid alpha-glucosidase to correct the biochemical and motor function deficits of presymptomatic and symptomatic Pompe mice’, Hum Gene Ther, 19: 609-21.

While the present inventions have been described and illustrated in conjunction with a number of specific embodiments, those skilled in the art will appreciate that variations and modifications may be made without departing from the principles of the inventions as herein illustrated, as described and claimed. The present inventions may be embodied in other specific forms without departing from their spirit or essential characteristics. The described embodiments are considered in all respects to be illustrative and not restrictive.

In closing, regarding the exemplary embodiments of the present invention as shown and described herein, it will be appreciated that a genomic construct, comprising an AAV (adeno-associated virus) viral virion is disclosed and configured for delivery of AAV vectors. Because the principles of the invention may be practiced in a number of configurations beyond those shown and described, it is to be understood that the invention is not in any way limited by the exemplary embodiments, but is generally directed to a genomic construct, comprising an AAV (adeno-associated virus) viral virion apparatus and is able to take numerous forms to do so without departing from the spirit and scope of the invention.

Certain embodiments of the present invention are described herein, including the best mode known to the inventor(s) for carrying out the invention. Of course, variations on these described embodiments will become apparent to those of ordinary skill in the art upon reading the foregoing description. The inventor(s) expect skilled artisans to employ such variations as appropriate, and the inventor(s) intend for the present invention to be practiced otherwise than specifically described herein. Accordingly, this invention includes all modifications and equivalents of the subject matter recited in the claims appended hereto as permitted by applicable law. Moreover, any combination of the above-described embodiments in all possible variations thereof is encompassed by the invention unless otherwise indicated herein or otherwise clearly contradicted by context.

Groupings of alternative embodiments, elements, or steps of the present invention are not to be construed as limitations. Each group member may be referred to and claimed individually or in any combination with other group members disclosed herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Unless otherwise indicated, all numbers expressing a characteristic, item, quantity, parameter, property, term, and so forth used in the present specification and claims are to be understood as being modified in all instances by the term “about.” As used herein, the term “about” means that the characteristic, item, quantity, parameter, property, or term so qualified encompasses a range of plus or minus ten percent above and below the value of the stated characteristic, item, quantity, parameter, property, or term. Accordingly, unless indicated to the contrary, the numerical parameters set forth in the specification and attached claims are approximations that may vary. At the very least, and not as an attempt to limit the application of the doctrine of equivalents to the scope of the claims, each numerical indication should at least be construed in light of the number of reported significant digits and by applying ordinary rounding techniques. Notwithstanding that the numerical ranges and values setting forth the broad scope of the invention are approximations, the numerical ranges and values set forth in the specific examples are reported as precisely as possible. Any numerical range or value, however, inherently contains certain errors necessarily resulting from the standard deviation found in their respective testing measurements. Recitation of numerical ranges of values herein is merely intended to serve as a shorthand method of referring individually to each separate numerical value falling within the range. Unless otherwise indicated herein, each individual value of a numerical range is incorporated into the present specification as if it were individually recited herein. Similarly, as used herein, unless indicated to the contrary, the term “substantially” is a term of degree intended to indicate an approximation of the characteristic, item, quantity, parameter, property, or term so qualified, encompassing a range that can be understood and construed by those of ordinary skill in the art.

Use of the terms “may” or “can” in reference to an embodiment or aspect of an embodiment also carries with it the alternative meaning of “may not” or “cannot.” As such, if the present specification discloses that an embodiment or an aspect of an embodiment may be or can be included as part of the inventive subject matter, then the negative limitation or exclusionary proviso is also explicitly meant, meaning that an embodiment or an aspect of an embodiment may not be or cannot be included as part of the inventive subject matter. In a similar manner, use of the term “optionally” in reference to an embodiment or aspect of an embodiment means that such embodiment or aspect of the embodiment may be included as part of the inventive subject matter or may not be included as part of the inventive subject matter. Whether such a negative limitation or exclusionary proviso applies will be based on whether the negative limitation or exclusionary proviso is recited in the claimed subject matter.

When used in the claims, whether as filed or added per amendment, the open-ended transitional term “comprising” (along with equivalent open-ended transitional phrases thereof such as “including,” “containing” and “having”) encompasses all the expressly recited elements, limitations, steps and/or features alone or in combination with un-recited subject matter; the named elements, limitations and/or features are essential, but other unnamed elements, limitations and/or features may be added and still form a construct within the scope of the claim. Specific embodiments disclosed herein may be further limited in the claims using the closed-ended transitional phrases “consisting of” or “consisting essentially of” in lieu of or as an amendment for “comprising.” When used in the claims, whether as filed or added per amendment, the closed-ended transitional phrase “consisting of” excludes any element, limitation, step, or feature not expressly recited in the claims. The closed-ended transitional phrase “consisting essentially of” limits the scope of a claim to the expressly recited elements, limitations, steps and/or features and any other elements, limitations, steps and/or features that do not materially affect the basic and novel characteristic(s) of the claimed subject matter. Thus, the meaning of the open-ended transitional phrase “comprising” is being defined as encompassing all the specifically recited elements, limitations, steps and/or features as well as any optional, additional unspecified ones. The meaning of the closed-ended transitional phrase “consisting of” is being defined as only including those elements, limitations, steps and/or features specifically recited in the claim, whereas the meaning of the closed-ended transitional phrase “consisting essentially of” is being defined as only including those elements, limitations, steps and/or features specifically recited in the claim and those elements, limitations, steps and/or features that do not materially affect the basic and novel characteristic(s) of the claimed subject matter. Therefore, the open-ended transitional phrase “comprising” (along with equivalent open-ended transitional phrases thereof) includes within its meaning, as a limiting case, claimed subject matter specified by the closed-ended transitional phrases “consisting of” or “consisting essentially of” As such, embodiments described herein or so claimed with the phrase “comprising” are expressly or inherently unambiguously described, enabled and supported herein for the phrases “consisting essentially of” and “consisting of.”

While aspects of the invention have been described with reference to at least one exemplary embodiment, it is to be clearly understood by those skilled in the art that the invention is not limited thereto. Rather, the scope of the invention is to be interpreted only in conjunction with the appended claims and it is made clear, here, that the inventor(s) believe that the claimed subject matter is the invention.

The references disclosed in the specification and Examples, including but not limited to patents and patent applications, and international patent applications are all incorporated herein in their entirety by reference.

All patents, patent publications, and other publications referenced and identified in the present specification are individually and expressly incorporated herein by reference in their entirety for the purpose of describing and disclosing, for example, the compositions and methodologies described in such publications that might be used in connection with the present invention. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

TABLE 9
SEQUENCES

| SEQ | |
|---|---|
| ID | |
| NO: | SEQUENCE |
| 1 | >GAA polypeptide \| ACTUS101 |
| | MGVRHPPCSHRLLAVCALVSLATAALLGHILLHDFLLVPRELSGSSPVLEETHPAHQQGASRPGPRDAQAHPGRPRAVPTQCDVPPNSRFDCAPDKAITQEQCEARGCCYIPAKQGLQGAQMGQPWCFFPPSYPSYKLENLSSSE |
| | MGYTATLTRTTPTFFPKDILTLRLDVMMETENRLHFTIKDPANRRYEVPLETPRVHSRAPSPLYSVEFSEEPFGVIVHRQLDGRVLLNTTVAPLFFADQFLQLSTSLPSQYITGLAEHLSPLMLSTSWTRITLWNRDLAPTPGAN |
| | LYGSHPFYLALEDGGSAHGVFLLNSNAMDVVLQPSPALSWRSTGGILDVYIFLGPEPKSVVQQYLDVVGYPFMPPYWGLGFHLCRWGYSSTAITRQVVENMTRAHFPLDVQWNDLDYMDSRRDFTFNKDGFRDFPAMVQELHQGG |
| | RRYMMIVDPAISSSGPAGSYRPYDEGLRRGVFITNETGQPLIGKVWPGSTAFPDFTNPTALAWWEDMVAEFHDQVPFDGMWIDMNEPSNFIRGSEDGCPNNELENPPYVPGVVGGTLQAATICASSHQFLSTHYNLHNLYGLTEA |
| | IASHRALVKARGTRPFVISRSTFAGHGRYAGHWTGDVWSSWEQLASSVPEILQFNLLGVPLVGADVCGFLGNTSEELCVRWTQLGAFYPFMRNHNSLLSLPQEPYSFSEPAQQAMRKALTLRYALLPHLYTLFHQAHVAGETVAR |
| | PLFLEFPKDSSTWTVDHQLLWGEALLITPVLQAGKAEVTGYFPLGTWYDLQTVPIEALGSLPPPPAAPREPAIHSEGQWVTLPAPLDTINVHLRAGYIIPLQGPGLTTTESRQQPMALAVALTKGGEARGELFWDDGESLEVLER |
| | GAYTQVIFLARNNTIVNELVRVTSEGAGLQLQKVTVLGVATAPQQVLSNGVPVSNFTYSPDTKVLDICVSLLMGEQFLVSWC*. |
| 2 | >ACTUS_101 GAA nucleic acid sequence |
| | ATGGGGGTGAGGCACCCTCCCTGTTCTCACAGGCTGCTGGCTGTGTGTGCTCTGGTGTCTCTGGCCACTGCTGCCCTGCTGGGCCATATCCTGCTGCATGACTTCCTGCTGGTGCCTAGGGAGCTGTCTGGCAGCAGCCCTGTGC |
| | TGGAAGAGACCCACCCTGCTCACCAGCAGGGGGCCAGCAGGCCAGGCCCCAGGGATGCCCAGGCCCACCCTGGCAGACCCAGGGCTGTGCCCACCCAGTGTGATGTGCCTCCCAACAGCAGATTTGACTGTGCCCCTGATAAGGC |
| | CATTACTCAGGAACAGTGTGAGGCCAGGGGCTGCTGCTATATCCCTGCCAAGCAGGGCCTGCAGGGGGCCCAGATGGGGCAGCCCTGGTGCTTCTTCCCCCCCAGCTACCCCTCTTATAAGCTGGAGAATCTGAGCAGCTCTGAA |
| | ATGGGCTACACTGCCACTCTGACCAGGACTACCCCCACCTTCTTCCCCAAGGATATTCTGACTCTGAGGCTGGATGTGATGATGGAGACTGAGAACAGGCTGCACTTCACCATCAAGGACCCTGCCAACAGGAGGTATGAGGTGC |
| | CCCTGGAAACTCCCAGGGTGCACTCTAGGGCCCCCAGCCCCCTGTATTCTGTGGAGTTCTCTGAGGAGCCTTTTGGGGTCATTGTCCACAGGCAGCTGGATGGCAGGGTGCTGCTGAATACTACTGTGGCCCCTCTGTTCTTTGC |
| | TGACCAGTTCCTGCAGCTGAGCACCAGCCTGCCTTCTCAGTACATTACTGGCCTGGCTGAGCATCTGAGCCCCCTGATGCTGAGCACCTCTTGGACCAGAATCACCCTGTGGAACAGGGACCTGGCTCCCACTCCTGGGGCCAAC |
| | CTGTATGGCAGCCACCCCTTCTACCTGGCCCTGGAGGATGGGGGCTCTGCCCATGGGGTGTTCCTGCTGAACAGCAATGCCATGGATGTGGTGCTGCAGCCCTCTCCTGCCCTGTCTTGGAGATCTACTGGGGGCATCCTGGATG |
| | TGTATATCTTCCTGGGGCCTGAGCCCAAGTCTGTGGTGCAGCAGTACCTGGATGTGGTGGGCTACCCCTTCATGCCCCCCTACTGGGGCCTGGGCTTCCACCTGTGCAGGTGGGGCTACTCTTCTACTGCTATCACCAGGCAGGT |
| | GGTGGAGAACATGACCAGGGCTCACTTCCCTCTGGATGTGCAGTGGAATGACCTGGACTACATGGACTCTAGGAGAGACTTCACTTTTAATAAGGATGGGTTCAGGGACTTTCCTGCCATGGTGCAGGAGCTGCATCAGGGGGGC |
| | AGGAGATATATGATGATTGTGGACCCTGCTATTTCTAGCTCTGGCCCTGCTGGCAGCTATAGGCCCTATGATGAGGGGCTGAGGAGGGGGGTGTTCATCACTAATGAGACTGGCCAGCCCCTGATTGGCAAGGTGTGGCCTGGCT |
| | CTACTGCCTTCCCTGATTTCACCAACCCCACTGCCCTGGCCTGGTGGGAGGATATGGTGGCTGAGTTTCATGACCAGGTGCCCTTTGATGGCATGTGGATTGACATGAATGAGCCCAGCAACTTTATCAGGGGCTCTGAAGATGG |
| | CTGCCCCAACAATGAGCTGGAGAACCCCCCCTATGTGCCTGGGGTGGTGGGGGGCACCCTGCAGGCTGCCACCATCTGTGCCAGCAGCCACCAGTTCCTGAGCACCCACTATAACCTGCACAACCTGTATGGCCTGACTGAGGCC |
| | ATTGCCTCTCACAGGGCCCTGGTGAAGGCTAGGGGGACTAGGCCCTTTGTGATCAGCAGGTCTACTTTTGCTGGCCATGGCAGGTATGCTGGGCACTGGACTGGGGATGTGTGGTCTAGCTGGGAGCAGCTGGCCAGCTCTGTGC |
| | CTGAGATCCTGCAGTTTAATCTGCTGGGGGTGCCCCTGGTGGGGGCTGATGTGTGTGGCTTCCTGGGCAATACCTCTGAGGAGCTGTGTGTGAGGTGGACTCAGCTGGGGGCTTTCTACCCCTTCATGAGAAACCACAACTCTCT |
| | GCTGAGCCTGCCCCAGGAGCCCTATTCTTTTTCTGAGCCTGCCCAGCAGGCTATGAGGAAGGCCCTGACTCTGAGGTATGCCCTGCTGCCCCACCTGTATACCCTGTTCCATCAGGCCCATGTGGCTGGGGAGACTGTGGCCAGA |
| | CCTCTGTTCCTGGAGTTCCCCAAGGATAGCTCTACTTGGACTGTGGACCACCAGCTGCTGTGGGGGGAGGCTCTGCTGATCACCCCTGTGCTGCAGGCTGGGAAGGCTGAGGTGACTGGCTATTTCCCCCTGGGCACCTGGTATG |
| | ATCTGCAGACTGTGCCCATTGAGGCCCTGGGCTCTCTGCCCCCTCCCCCTGCTGCCCCCAGGGAGCCTGCCATCCACTCTGAGGGCCAGTGGGTGACCCTGCCTGCCCCTCTGGACACTATCAATGTGCACCTGAGGGCTGGCTA |
| | CATCATCCCCCTGCAGGGCCCTGGCCTGACTACCACTGAGTCTAGGCAGCAGCCCATGGCCCTGGCTGTGGCTCTGACCAAGGGGGGGGAGGCCAGGGGGGAGCTGTTCTGGGATGATGGGGAGTCTCTGGAGGTGCTGGAGAGG |
| | GGGGCCTACACCCAGGTGATCTTCCTGGCTAGGAATAACACCATTGTCAATGAGCTGGTGAGGGTGACCTCTGAGGGGGCTGGCCTGCAGCTGCAGAAGGTGACTGTGCTGGGGGTGGCTACTGCCCCCCAGCAGGTGCTGAGCA |
| | ATGGGGTGCCTGTGAGCAACTTCACCTATAGCCCTGACACCAAGGTGCTGGACATCTGTGTGAGCCTGCTGATGGGGGAGCAGTTCCTGGTGAGCTGGTGCTAA |
| 3 | > coGAA nucleic acid |
| | ATGGGGGTGAGGCACCCTCCCTGTTCTCACAGGCTGCTGGCTGTGTGTGCTCTGGTGTCTCTGGCCACTGCTGCCCTGCTGGGCCATATCCTGCTGCATGACTTCCTGCTGGTGCCTAGGGAGCTGTCTGGCAGCAGCCCTGTGC |
| | TGGAAGAGACCCACCCTGCTCACCAGCAGGGGGCCAGCAGGCCAGGCCCCAGGGATGCCCAGGCCCACCCTGGCAGACCCAGGGCTGTGCCCACCCAGTGTGATGTGCCTCCCAACAGCAGATTTGACTGTGCCCCTGATAAGGC |
| | CATTACTCAGGAACAGTGTGAGGCCAGGGGCTGCTGCTATATCCCTGCCAAGCAGGGCCTGCAGGGGGCCCAGATGGGGCAGCCCTGGTGCTTCTTCCCCCCCAGCTACCCCTCTTATAAGCTGGAGAATCTGAGCAGCTCTGAA |
| | ATGGGCTACACTGCCACTCTGACCAGGACTACCCCCACCTTCTTCCCCAAGGATATTCTGACTCTGAGGCTGGATGTGATGATGGAGACTGAGAACAGGCTGCACTTCACCATCAAGGACCCTGCCAACAGGAGGTATGAGGTGC |
| | CCCTGGAAACTCCCAGGGTGCACTCTAGGGCCCCCAGCCCCCTGTATTCTGTGGAGTTCTCTGAGGAGCCTTTTGGGGTCATTGTCCACAGGCAGCTGGATGGCAGGGTGCTGCTGAATACTACTGTGGCCCCTCTGTTCTTTGC |
| | TGACCAGTTCCTGCAGCTGAGCACCAGCCTGCCTTCTCAGTACATTACTGGCCTGGCTGAGCATCTGAGCCCCCTGATGCTGAGCACCTCTTGGACCAGAATCACCCTGTGGAACAGGGACCTGGCTCCCACTCCTGGGGCCAAC |
| | CTGTATGGCAGCCACCCCTTCTACCTGGCCCTGGAGGATGGGGGCTCTGCCCATGGGGTGTTCCTGCTGAACAGCAATGCCATGGATGTGGTGCTGCAGCCCTCTCCTGCCCTGTCTTGGAGATCTACTGGGGGCATCCTGGATG |
| | TGTATATCTTCCTGGGGCCTGAGCCCAAGTCTGTGGTGCAGCAGTACCTGGATGTGGTGGGCTACCCCTTCATGCCCCCCTACTGGGGCCTGGGCTTCCACCTGTGCAGGTGGGGCTACTCTTCTACTGCTATCACCAGGCAGGT |
| | GGTGGAGAACATGACCAGGGCTCACTTCCCTCTGGATGTGCAGTGGAATGACCTGGACTACATGGACTCTAGGAGAGACTTCACTTTTAATAAGGATGGGTTCAGGGACTTTCCTGCCATGGTGCAGGAGCTGCATCAGGGGGGC |
| | AGGAGATATATGATGATTGTGGACCCTGCTATTTCTAGCTCTGGCCCTGCTGGCAGCTATAGGCCCTATGATGAGGGGCTGAGGAGGGGGGTGTTCATCACTAATGAGACTGGCCAGCCCCTGATTGGCAAGGTGTGGCCTGGCT |
| | CTACTGCCTTCCCTGATTTCACCAACCCCACTGCCCTGGCCTGGTGGGAGGATATGGTGGCTGAGTTTCATGACCAGGTGCCCTTTGATGGCATGTGGATTGACATGAATGAGCCCAGCAACTTTATCAGGGGCTCTGAAGATGG |
| | CTGCCCCAACAATGAGCTGGAGAACCCCCCCTATGTGCCTGGGGTGGTGGGGGGCACCCTGCAGGCTGCCACCATCTGTGCCAGCAGCCACCAGTTCCTGAGCACCCACTATAACCTGCACAACCTGTATGGCCTGACTGAGGCC |
| | ATTGCCTCTCACAGGGCCCTGGTGAAGGCTAGGGGGACTAGGCCCTTTGTGATCAGCAGGTCTACTTTTGCTGGCCATGGCAGGTATGCTGGGCACTGGACTGGGGATGTGTGGTCTAGCTGGGAGCAGCTGGCCAGCTCTGTGC |
| | CTGAGATCCTGCAGTTTAATCTGCTGGGGGTGCCCCTGGTGGGGGCTGATGTGTGTGGCTTCCTGGGCAATACCTCTGAGGAGCTGTGTGTGAGGTGGACTCAGCTGGGGGCTTTCTACCCCTTCATGAGAAACCACAACTCTCT |
| | GCTGAGCCTGCCCCAGGAGCCCTATTCTTTTTCTGAGCCTGCCCAGCAGGCTATGAGGAAGGCCCTGACTCTGAGGTATGCCCTGCTGCCCCACCTGTATACCCTGTTCCATCAGGCCCATGTGGCTGGGGAGACTGTGGCCAGA |
| | CCTCTGTTCCTGGAGTTCCCCAAGGATAGCTCTACTTGGACTGTGGACCACCAGCTGCTGTGGGGGGAGGCTCTGCTGATCACCCCTGTGCTGCAGGCTGGGAAGGCTGAGGTGACTGGCTATTTCCCCCTGGGCACCTGGTATG |
| | ATCTGCAGACTGTGCCCATTGAGGCCCTGGGCTCTCTGCCCCCTCCCCCTGCTGCCCCCAGGGAGCCTGCCATCCACTCTGAGGGCCAGTGGGTGACCCTGCCTGCCCCTCTGGACACTATCAATGTGCACCTGAGGGCTGGCTA |
| | CATCATCCCCCTGCAGGGCCCTGGCCTGACTACCACTGAGTCTAGGCAGCAGCCCATGGCCCTGGCTGTGGCTCTGACCAAGGGGGGGGAGGCCAGGGGGGAGCTGTTCTGGGATGATGGGGAGTCTCTGGAGGTGCTGGAGAGG |
| | GGGGCCTACACCCAGGTGATCTTCCTGGCTAGGAATAACACCATTGTCAATGAGCTGGTGAGGGTGACCTCTGAGGGGGCTGGCCTGCAGCTGCAGAAGGTGACTGTGCTGGGGGTGGCTACTGCCCCCCAGCAGGTGCTGAGCA |
| | ATGGGGTGCCTGTGAGCAACTTCACCTATAGCCCTGACACCAAGGTGCTGGACATCTGTGTGAGCCTGCTGATGGGGGAGCAGTTCCTGGTGAGCTGGTGCTAA |
| 4 | > coGAA GAA nucleic acid |
| | ATGGGAGTGAGACATCCTCCTTGTTCTCATAGACTGCTGGCTGTGTGTGCTCTGGTGTCTCTGGCTACAGCTGCTCTGCTGGGACATATTCTGCTGCATGATTTTCTGCTAGTGCCTAGAGAGCTGTCTGGATCTTCTCCTGTGC |
| | TGGAGGAGACACATCCTGCTCATCAGCAGGGAGCTTCTAGACCTGGACCTAGAGATGCTCAGGCTCATCCTGGAAGACCTAGAGCTGTGCCTACACAGTGTGATGTGCCTCCTAATTCTAGATTTGATTGTGCTCCTGATAAGGC |
| | TATAACACAGGAGCAGTGTGAGGCTAGAGGATGTTGTTATATTCCTGCTAAGCAGGGACTGCAGGGAGCTCAGATGGGACAGCCTTGGTGTTTTTTTCCTCCTTCTTATCCTTCTTATAAGCTGGAGAATCTGTCTTCTTCTGAG |
| | ATGGGATATACAGCTACACTGACAAGAACAACACCTACATTTTTTCCTAAGGATATTCTGACACTGAGACTGGATGTGATGATGGAGACAGAGAATAGACTGCATTTTACAATTAAGGATCCTGCTAATAGAAGATATGAGGTGC |
| | CTCTAGAGACACCTAGAGTGCATTCTAGAGCTCCTTCTCCTCTGTATTCTGTGGAGTTTTCTGAGGAGCCTTTTGGAGTGATTGTGCATAGACAGCTGGATGGAAGAGTGCTGCTGAATACAACAGTGGCTCCTCTGTTTTTTGC |
| | TGATCAGTTTCTGCAGCTGTCTACATCTCTGCCTTCTCAGTATATTACAGGACTGGCTGAGCATCTGTCTCCTCTGATGCTGTCTACATCTTGGACAAGAATTACACTGTGGAATAGAGATCTGGCTCCTACACCTGGAGCTAAT |
| | CTGTATGGATCTCATCCTTTTTATCTGGCTCTGGAGGATGGAGGATCTGCTCATGGAGTGTTTCTGCTGAATTCTAATGCTATGGATGTAGTGCTGCAGCCTTCTCCTGCTCTGTCTTGGAGATCTACAGGAGGAATTCTGGATG |
| | TGTATATTTTTCTAGGACCTGAGCCTAAGTCTGTGGTGCAGCAGTATCTGGATGTAGTGGGATATCCTTTTATGCCTCCTTATTGGGGACTGGGATTTCATCTGTGTAGATGGGGATATTCTTCTACAGCTATTACAAGACAGGT |
| | GGTTGAGAATATGACAAGAGCTCATTTTCCTCTGGATGTGCAGTGGAATGATCTAGATTATATGGATTCTAGAAGAGATTTTACATTTAATAAGGATGGATTTAGAGATTTTCCTGCTATGGTGCAGGAGCTGCATCAGGGAGGA |
| | AGAAGATATATGATGATTGTGGATCCTGCTATTTCTTCTTCTGGACCTGCTGGATCTTATAGACCTTATGATGAGGGACTGAGAAGAGGAGTGTTTATTACAAATGAGACAGGACAGCCTCTGATTGGAAAGGTGTGGCCTGGAT |
| | CTACAGCTTTTCCTGATTTTACAAATCCTACAGCTCTGGCTTGGTGGGAGGATATGGTGGCTGAGTTTCATGATCAGGTGCCTTTTGATGGAATGTGGATTGATATGAATGAGCCTTCTAATTTTATAAGAGGATCTGAGGATGG |
| | ATGTCCTAATAATGAGCTAGAGAATCCTCCTTATGTGCCTGGAGTAGTGGGAGGAACACTGCAGGCTGCTACAATTTGTGCTTCTTCTCATCAGTTTCTGTCTACACATTATAATCTGCATAATCTGTATGGACTGACAGAGGCT |
| | ATTGCTTCTCATAGAGCTCTGGTGAAGGCTAGAGGAACAAGACCTTTTGTGATTTCTAGATCTACATTTGCTGGACATGGAAGATATGCTGGACATTGGACAGGAGATGTATGGTCTTCTTGGGAGCAGCTAGCTTCTTCTGTGC |
| | CTGAGATTCTGCAGTTTAATCTGCTAGGAGTGCCTCTGGTGGGAGCTGATGTGTGTGGATTTCTGGGAAATACATCTGAGGAGCTGTGTGTGAGATGGACACAGCTGGGAGCTTTTTATCCTTTTATGAGAAATCATAATTCTCT |
| | GCTGTCTCTGCCTCAGGAGCCTTATTCTTTTTCTGAGCCTGCTCAGCAGGCTATGAGAAAGGCTCTGACACTGAGATATGCTCTGCTGCCTCATCTGTATACACTGTTTCATCAGGCTCATGTGGCTGGAGAGACAGTAGCTAGA |
| | CCTCTGTTTCTGGAGTTTCCTAAGGATTCTTCTACATGGACAGTTGATCATCAGCTGCTGTGGGGAGAGGCTCTGCTGATTACACCTGTGCTGCAGGCTGGAAAGGCTGAGGTGACAGGATATTTTCCTCTGGGAACATGGTATG |
| | ATCTGCAGACAGTGCCTATAGAGGCTCTGGGATCTCTGCCTCCTCCTCCTGCTGCTCCTAGAGAGCCTGCTATTCATTCTGAGGGACAGTGGGTGACACTGCCTGCTCCTCTGGATACAATTAATGTGCATCTGAGAGCTGGATA |
| | TATTATTCCTCTGCAGGGACCTGGACTGACAACAACAGAGTCTAGACAGCAGCCTATGGCTCTGGCTGTGGCTCTGACAAAGGGAGGAGAGGCTAGAGGAGAGCTGTTTTGGGATGATGGAGAGTCTCTGGAGGTGCTGGAGAGA |
| | GGAGCTTATACACAGGTGATTTTTCTGGCTAGAAATAATACAATTGTGAATGAGCTGGTGAGAGTGACATCTGAGGGAGCTGGACTGCAGCTGCAGAAGGTGACAGTGCTGGGAGTGGCTACAGCTCCTCAGCAGGTGCTGTCTA |
| | ATGGAGTGCCTGTGTCTAATTTTACATATTCTCCTGATACAAAGGTGCTGGATATTTGTGTGTCTCTGCTGATGGGAGAGCAGTTTCTGGTGTCTTGGTGT |
| 5 | > coGAA GAA nucleic acid |
| | ATGGGGGTGAGACACCCCCCTTGCTCTCACAGGCTGCTGGCTGTGTGTGCCCTGGTGAGCCTGGCCACTGCTGCTCTGCTGGGCCACATCCTGCTGCATGACTTCCTGCTGGTGCCAAGAGAGCTGTCTGGGAGCAGCCCTGTGC |
| | TGGAGGAGACCCACCCTGCCCATCAGCAGGGGGCCAGCAGGCCAGGCCCTAGGGATGCTCAGGCTCACCCAGGGAGGCCTAGGGCTGTGCCCACCCAGTGTGATGTGCCCCCTAACAGCAGGTTTGACTGTGCTCCAGACAAGGC |
| | CATCACCCAGGAGCAGTGTGAGGCCAGAGGCTGCTGCTACATTCCTGCTAAGCAGGGCCTGCAGGGGGCTCAGATGGGCCAGCCCTGGTGCTTTTTCCCCCCTAGCTACCCTAGCTACAAGCTGGAGAACCTGAGCAGCTCTGAG |
| | ATGGGGTATACTGCCACTCTGACCAGGACCACCCCCACCTTCTTTCCCAAGGATATTCTGACCCTGAGGCTGGATGTGATGATGGAGACTGAGAACAGGCTGCACTTCACCATCAAGGATCCTGCCAACAGGAGGTATGAGGTGC |
| | CTCTGGAAACCCCCAGGGTCCACAGCAGGGCCCCTAGCCCTCTGTACTCTGTGGAGTTCTCTGAGGAGCCCTTTGGGGTGATTGTGCATAGGCAGCTGGATGGCAGGGTGCTGCTGAATACCACTGTGGCCCCCCTGTTCTTTGC |
| | TGACCAGTTCCTGCAGCTGAGCACCTCTCTGCCCTCTCAGTACATCACTGGCCTGGCTGAGCACCTGAGCCCTCTGATGCTGAGCACCTCTTGGACCAGGATCACCCTGTGGAATAGGGATCTGGCCCCTACCCCTGGGGCCAAC |
| | CTGTATGGGAGCCACCCTTTCTACCTGGCCCTGGAGGATGGGGGGTCTGCCCATGGGGTGTTCCTGCTGAACAGCAATGCCATGGATGTGGTGCTGCAGCCCAGCCCTGCCCTGTCTTGGAGGTCTACTGGGGGCATCCTGGATG |
| | TGTATATCTTCCTGGGCCCTGAGCCCAAGTCTGTGGTGCAGCAGTACCTGGATGTGGTGGGCTACCCCTTCATGCCCCCCTACTGGGGCCTGGGCTTCCACCTGTGCAGGTGGGGCTACAGCAGCACTGCCATCACCAGGCAGGT |
| | GGTGGAGAACATGACTAGGGCCCACTTCCCCCTGGATGTGCAGTGGAATGACCTGGACTACATGGACTCTAGGAGGGATTTCACCTTTAACAAGGATGGGTTTAGGGACTTCCCTGCTATGGTGCAGGAGCTGCATCAGGGGGGC |
| | AGGAGGTATATGATGATTGTGGACCCTGCCATCTCTAGCTCTGGGCCTGCTGGCAGCTACAGGCCCTATGATGAGGGCCTGAGGAGGGGGGTGTTCATCACTAATGAAACTGGCCAGCCCCTGATTGGGAAGGTGTGGCCTGGCT |
| | CTACTGCTTTCCCTGACTTCACCAACCCCACTGCCCTGGCCTGGTGGGAGGACATGGTGGCTGAGTTCCATGATCAGGTGCCCTTTGATGGCATGTGGATTGACATGAATGAGCCTAGCAACTTCATTAGGGGCTCTGAGGATGG |
| | CTGCCCCAACAATGAGCTGGAGAACCCCCCCTATGTGCCTGGGGTGGTGGGGGGGACTCTGCAGGCTGCCACTATTTGTGCTTCTTCTCACCAGTTTCTGAGCACCCACTATAACCTGCATAACCTGTATGGGCTGACTGAGGCT |
| | ATTGCCAGCCACAGGGCCCTGGTGAAGGCCAGGGGCACTAGGCCTTTTGTGATCTCTAGGAGCACCTTTGCTGGCCATGGCAGGTATGCTGGCCATTGGACTGGGGATGTGTGGAGCAGCTGGGAGCAGCTGGCCAGCTCTGTCC |
| | CTGAGATCCTGCAGTTTAATCTGCTGGGGGTGCCCCTGGTGGGGGCTGATGTGTGTGGCTTCCTGGGCAACACCTCTGAAGAGCTGTGTGTGAGGTGGACCCAGCTGGGGGCCTTCTACCCCTTCATGAGGAACCACAATAGCCT |
| | GCTGAGCCTGCCCCAGGAGCCCTACAGCTTTTCTGAGCCAGCCCAGCAGGCCATGAGGAAGGCCCTGACCCTGAGGTATGCCCTGCTGCCTCACCTGTACACCCTGTTCCACCAGGCCCATGTGGCTGGGGAGACTGTGGCCAGG |
| | CCCCTGTTCCTGGAGTTTCCCAAAGATAGCAGCACCTGGACTGTGGACCACCAGCTGCTGTGGGGGGAGGCCCTGCTGATCACCCCAGTGCTGCAGGCTGGGAAGGCTGAGGTGACTGGGTACTTCCCCCTGGGCACCTGGTATG |
| | ACCTGCAGACTGTGCCTATTGAGGCTCTGGGCAGCCTGCCCCCTCCCCCTGCTGCCCCCAGAGAGCCTGCCATCCATTCTGAGGGGCAGTGGGTGACTCTGCCTGCCCCCCTGGACACCATCAATGTGCACCTGAGAGCTGGGTA |
| | TATCATCCCCCTGCAGGGCCCTGGGCTGACCACTACTGAGTCTAGGCAGCAGCCCATGGCTCTGGCTGTGGCCCTGACTAAGGGGGGGGAGGCTAGGGGGGAGCTGTTCTGGGATGATGGGGAGTCTCTGGAGGTGCTGGAGAGG |
| | GGGGCCTACACCCAGGTGATCTTCCTGGCTAGGAACAACACCATTGTGAATGAGCTGGTGAGGGTGACTTCTGAGGGGGCTGGCCTGCAGCTGCAGAAGGTGACTGTGCTGGGGGTGGCCACTGCCCCCCAGCAGGTCCTGAGCA |
| | ATGGGGTGCCTGTGAGCAACTTCACCTACTCTCCTGACACCAAGGTGCTGGACATCTGTGTGAGCCTGCTGATGGGGGAGCAGTTCCTGGTGAGCTGGTGCTGA |
| 6 | > coGAA GAA nucleic acid |
| | ATGGGGGTGAGGCACCCCCCCTGTTCTCACAGGCTGCTGGCTGTGTGTGCCCTGGTGAGCCTGGCTACTGCTGCCCTGCTGGGCCACATCCTGCTGCATGACTTCCTGCTGGTGCCCAGGGAGCTGTCTGGCTCTAGCCCAGTGC |
| | TGGAGGAAACCCATCCTGCCCACCAGCAGGGGGCCAGCAGGCCTGGCCCTAGGGATGCCCAGGCTCATCCTGGGAGGCCCAGGGCTGTGCCCACCCAGTGTGATGTGCCTCCCAACTCTAGATTTGACTGTGCCCCTGACAAGGC |
| | CATCACTCAGGAGCAGTGTGAGGCCAGGGGGTGCTGCTACATCCCTGCCAAGCAGGGGCTGCAGGGGGCCCAGATGGGCCAGCCTTGGTGCTTCTTCCCTCCCAGCTACCCCTCTTACAAGCTGGAGAACCTGAGCAGCTCTGAG |
| | ATGGGGTACACTGCTACCCTGACTAGGACCACCCCCACCTTCTTCCCTAAGGATATCCTGACCCTGAGACTGGATGTGATGATGGAGACTGAGAACAGGCTGCACTTCACTATTAAGGACCCTGCTAACAGGAGGTATGAGGTGC |
| | CCCTGGAGACCCCCAGGGTGCACAGCAGGGCCCCCAGCCCCCTGTACTCTGTGGAGTTCTCTGAGGAGCCCTTTGGGGTGATTGTGCACAGGCAGCTGGATGGCAGAGTGCTGCTGAACACCACTGTGGCTCCTCTGTTCTTTGC |
| | TGACCAGTTCCTGCAGCTGAGCACTAGCCTGCCCAGCCAGTACATCACTGGGCTGGCTGAGCACCTGTCTCCTCTGATGCTGAGCACTAGCTGGACTAGGATCACCCTGTGGAACAGGGACCTGGCCCCCACTCCTGGGGCTAAT |
| | CTGTATGGCAGCCACCCTTTTTACCTGGCCCTGGAGGATGGGGGGTCTGCCCATGGGGTGTTCCTGCTGAACTCTAATGCCATGGATGTGGTGCTGCAGCCCTCTCCTGCCCTGAGCTGGAGGAGCACTGGGGGCATCCTGGATG |
| | TGTACATCTTCCTGGGCCCTGAGCCCAAGTCTGTGGTGCAGCAGTATCTGGATGTGGTGGGCTATCCCTTTATGCCCCCCTATTGGGGCCTGGGGTTTCACCTGTGCAGGTGGGGGTATTCTTCTACTGCCATCACCAGGCAGGT |
| | GGTGGAGAACATGACCAGGGCCCACTTCCCCCTGGATGTGCAGTGGAATGACCTGGACTATATGGACAGCAGGAGGGACTTCACTTTCAACAAGGATGGGTTCAGGGACTTCCCTGCTATGGTGCAGGAGCTGCACCAGGGGGGG |
| | AGGAGGTACATGATGATTGTGGATCCTGCCATCTCTAGCTCTGGCCCTGCTGGCAGCTACAGGCCATATGATGAGGGCCTGAGGAGGGGGGTGTTCATCACCAATGAGACTGGGCAGCCTCTGATTGGCAAGGTGTGGCCTGGCA |
| | GCACTGCTTTCCCTGATTTCACCAACCCCACTGCTCTGGCCTGGTGGGAGGACATGGTGGCTGAGTTTCATGATCAGGTGCCTTTTGATGGGATGTGGATTGATATGAATGAGCCTAGCAACTTCATCAGAGGCTCTGAGGATGG |
| | CTGTCCCAATAATGAGCTGGAGAACCCCCCCTATGTGCCTGGGGTGGTGGGGGGCACCCTGCAGGCTGCTACCATCTGTGCTAGCTCTCATCAGTTCCTGAGCACTCACTACAACCTGCACAACCTGTATGGGCTGACTGAGGCT |
| | ATTGCCAGCCATAGGGCCCTGGTGAAGGCCAGAGGCACCAGGCCCTTTGTGATCAGCAGGAGCACCTTTGCTGGCCATGGCAGGTATGCTGGCCACTGGACTGGGGATGTGTGGAGCAGCTGGGAGCAGCTGGCCTCTTCTGTGC |
| | CTGAGATCCTGCAGTTCAACCTGCTGGGGGTGCCCCTGGTGGGGGCTGATGTGTGTGGCTTCCTGGGCAACACCTCTGAGGAGCTGTGTGTGAGGTGGACTCAGCTGGGGGCTTTTTATCCCTTCATGAGGAACCACAATAGCCT |
| | GCTGTCTCTGCCCCAGGAGCCCTATTCTTTCTCTGAGCCTGCCCAGCAGGCCATGAGGAAGGCCCTGACTCTGAGGTATGCCCTGCTGCCCCACCTGTACACCCTGTTCCACCAGGCCCATGTGGCTGGGGAGACTGTGGCCAGG |
| | CCCCTGTTTCTGGAGTTCCCTAAGGACAGCTCTACCTGGACTGTGGATCACCAGCTGCTGTGGGGGGAGGCCCTGCTGATCACTCCAGTCCTGCAGGCTGGCAAAGCTGAGGTGACTGGCTACTTCCCCCTGGGCACCTGGTATG |
| | ACCTGCAGACTGTGCCCATTGAGGCTCTGGGCAGCCTGCCCCCCCCCCCTGCTGCTCCTAGGGAGCCTGCCATTCACTCTGAGGGCCAGTGGGTGACTCTGCCTGCTCCCCTGGATACCATCAATGTGCATCTGAGGGCTGGCTA |
| | CATCATCCCCCTGCAGGGGCCTGGCCTGACCACCACTGAGAGCAGGCAGCAGCCCATGGCCCTGGCTGTGGCCCTGACTAAGGGGGGGGAGGCTAGGGGGGAGCTGTTCTGGGATGATGGGGAGTCTCTGGAAGTGCTGGAGAGG |
| | GGGGCTTACACCCAGGTGATTTTCCTGGCCAGGAACAACACTATTGTGAATGAGCTGGTGAGGGTGACCTCTGAGGGGGCTGGCCTGCAGCTGCAGAAGGTGACTGTGCTGGGGGTGGCCACTGCCCCCCAGCAGGTGCTGAGCA |
| | ATGGGGTGCCTGTGAGCAACTTTACCTACTCCCCTGATACCAAGGTGCTGGACATCTGTGTGTCTCTGCTGATGGGGGAACAGTTTCTGGTGTCTTGGTGCTGA |
| 7 | > coGAA GAA nucleic acid |
| | ATGGGGGTGAGGCACCCCCCCTGCTCTCACAGGCTGCTGGCTGTGTGTGCCCTGGTGAGCCTGGCCACTGCTGCTCTGCTGGGGCACATCCTGCTGCATGACTTCCTGCTGGTGCCCAGGGAGCTGTCTGGCAGCTCTCCTGTGC |
| | TGGAGGAAACTCATCCTGCCCATCAGCAGGGGGCCTCTAGGCCAGGCCCCAGGGATGCCCAGGCCCATCCTGGCAGGCCCAGAGCTGTGCCCACCCAGTGTGATGTGCCCCCCAACAGCAGGTTTGATTGTGCCCCTGATAAGGC |
| | CATCACCCAGGAGCAGTGTGAGGCCAGGGGCTGCTGCTACATCCCTGCCAAGCAGGGGCTGCAGGGGGCCCAGATGGGCCAGCCCTGGTGCTTCTTCCCCCCTTCTTACCCCAGCTACAAGCTGGAGAACCTGTCTTCTTCTGAG |
| | ATGGGCTATACTGCCACCCTGACCAGGACCACTCCCACCTTCTTCCCCAAGGACATCCTGACTCTGAGACTGGATGTGATGATGGAGACTGAGAACAGGCTGCACTTCACCATCAAGGACCCTGCCAACAGGAGGTATGAGGTGC |
| | CTCTGGAGACTCCTAGAGTGCACAGCAGGGCTCCCTCTCCCCTGTACTCTGTGGAGTTCTCTGAGGAGCCCTTTGGGGTGATTGTGCACAGACAGCTGGATGGGAGGGTGCTGCTGAACACCACTGTGGCCCCCCTGTTCTTTGC |
| | TGACCAGTTCCTGCAGCTGTCTACCTCTCTGCCCAGCCAGTACATTACTGGGCTGGCTGAACATCTGTCTCCCCTGATGCTGAGCACCAGCTGGACCAGGATCACTCTGTGGAACAGGGACCTGGCCCCCACTCCTGGGGCCAAT |
| | CTGTATGGGAGCCACCCATTCTACCTGGCCCTGGAGGATGGGGGCTCTGCCCATGGGGTGTTCCTGCTGAACAGCAATGCCATGGATGTGGTGCTGCAGCCTTCCCCTGCCCTGAGCTGGAGGTCTACTGGGGGCATCCTGGATG |
| | TGTACATTTTCCTGGGCCCTGAGCCCAAGTCTGTGGTGCAGCAGTACCTGGATGTGGTGGGGTACCCTTTCATGCCTCCCTACTGGGGCCTGGGCTTTCACCTGTGCAGGTGGGGCTATTCTAGCACTGCCATCACCAGGCAGGT |
| | GGTGGAGAACATGACTAGGGCTCATTTCCCCCTGGATGTGCAGTGGAATGATCTGGATTATATGGACTCTAGGAGGGACTTTACTTTCAATAAGGATGGCTTCAGGGATTTTCCTGCTATGGTGCAGGAGCTGCACCAGGGGGGC |
| | AGGAGGTATATGATGATTGTGGATCCTGCCATCTCTAGCTCTGGGCCTGCTGGCTCTTACAGGCCCTATGATGAGGGGCTGAGGAGGGGGGTGTTTATTACTAATGAGACTGGCCAGCCTCTGATTGGGAAGGTGTGGCCTGGCA |
| | GCACTGCCTTCCCAGACTTCACCAACCCCACTGCCCTGGCCTGGTGGGAGGACATGGTGGCTGAGTTCCATGACCAGGTGCCCTTTGATGGGATGTGGATTGATATGAATGAGCCCTCTAACTTCATTAGGGGCTCTGAGGATGG |
| | CTGCCCCAACAATGAGCTGGAGAACCCCCCATATGTGCCTGGGGTGGTGGGGGGGACCCTGCAGGCTGCTACCATCTGTGCTAGCTCTCACCAGTTCCTGAGCACCCACTACAACCTGCACAATCTGTATGGCCTGACTGAGGCC |
| | ATTGCCAGCCATAGGGCCCTGGTGAAGGCCAGGGGGACCAGACCCTTTGTGATCTCCAGGAGCACCTTTGCTGGCCATGGGAGATATGCTGGCCATTGGACTGGGGATGTGTGGTCTAGCTGGGAGCAGCTGGCCAGCTCTGTGC |
| | CTGAGATCCTGCAGTTCAATCTGCTGGGGGTGCCTCTGGTGGGGGCTGATGTGTGTGGCTTCCTGGGCAACACCTCTGAAGAACTGTGTGTGAGATGGACCCAGCTGGGGGCCTTCTACCCCTTCATGAGGAACCACAACAGCCT |
| | GCTGTCTCTGCCCCAGGAGCCTTACTCTTTTTCTGAGCCTGCCCAGCAGGCCATGAGGAAGGCCCTGACCCTGAGATATGCCCTGCTGCCTCACCTGTACACCCTGTTCCACCAGGCCCATGTGGCTGGGGAGACTGTGGCCAGG |
| | CCCCTGTTCCTGGAGTTCCCCAAGGACAGCAGCACCTGGACTGTGGACCACCAGCTGCTGTGGGGGGAGGCCCTGCTGATCACCCCTGTGCTGCAGGCTGGGAAGGCTGAGGTGACTGGCTACTTCCCCCTGGGCACCTGGTATG |
| | ATCTGCAGACTGTGCCCATTGAGGCCCTGGGGTCTCTGCCCCCCCCCCCTGCTGCTCCCAGAGAGCCTGCCATCCATTCTGAGGGCCAGTGGGTGACCCTGCCTGCCCCCCTGGACACCATTAATGTGCACCTGAGGGCTGGCTA |
| | CATTATCCCCCTGCAGGGCCCTGGCCTGACCACCACTGAGAGCAGGCAGCAGCCTATGGCTCTGGCTGTGGCCCTGACCAAGGGGGGGGAGGCCAGGGGGGAGCTGTTTTGGGATGATGGGGAGAGCCTGGAGGTGCTGGAGAGG |
| | GGGGCCTACACCCAGGTGATCTTCCTGGCCAGGAACAACACTATTGTGAATGAACTGGTGAGGGTCACTTCTGAGGGGGCTGGGCTGCAGCTGCAGAAGGTGACTGTGCTGGGGGTGGCCACTGCTCCCCAGCAGGTGCTGAGCA |
| | ATGGGGTGCCTGTGAGCAACTTCACCTACAGCCCTGATACCAAGGTGCTGGACATCTGTGTGAGCCTGCTGATGGGGGAGCAGTTCCTGGTGAGCTGGTGCTGA |
| 8 | > coGAAAA GAA nucleic acid |
| | ATGGGGGTGAGGCACCCTCCCTGCAGCCACAGGCTGCTGGCTGTGTGTGCTCTGGTGAGCCTGGCCACTGCTGCCCTGCTGGGCCACATTCTGCTGCATGATTTCCTGCTGGTGCCCAGGGAACTGTCTGGCAGCTCTCCTGTGC |
| | TGGAGGAGACCCATCCTGCCCACCAGCAGGGGGCCAGCAGGCCTGGCCCTAGGGATGCTCAGGCCCATCCTGGCAGGCCCAGGGCTGTGCCCACTCAGTGTGATGTGCCACCCAACAGCAGGTTTGACTGTGCCCCTGACAAGGC |
| | CATCACCCAGGAGCAGTGTGAGGCCAGGGGCTGCTGCTACATCCCTGCTAAGCAGGGCCTGCAGGGGGCCCAGATGGGGCAGCCCTGGTGCTTCTTCCCCCCCAGCTATCCCAGCTATAAGCTGGAGAACCTGAGCTCTTCTGAG |
| | ATGGGCTACACTGCCACCCTGACCAGGACCACCCCCACCTTCTTCCCCAAGGACATCCTGACCCTGAGGCTGGATGTGATGATGGAGACTGAGAACAGGCTGCACTTCACCATCAAGGACCCTGCCAATAGGAGGTATGAGGTGC |
| | CCCTGGAGACCCCCAGGGTGCACAGCAGGGCCCCCTCTCCCCTGTACTCTGTGGAGTTTTCTGAAGAGCCCTTTGGGGTGATTGTGCACAGGCAGCTGGATGGCAGGGTGCTGCTGAACACCACTGTGGCTCCCCTGTTCTTTGC |
| | TGACCAGTTCCTGCAGCTGAGCACCTCTCTGCCCAGCCAGTATATCACTGGGCTGGCTGAGCATCTGAGCCCCCTGATGCTGAGCACCAGCTGGACCAGGATCACCCTGTGGAATAGGGACCTGGCCCCTACCCCTGGGGCCAAC |
| | CTGTATGGCAGCCACCCTTTCTACCTGGCCCTGGAGGATGGGGGGTCTGCTCATGGGGTGTTCCTGCTGAACAGCAATGCCATGGATGTGGTGCTGCAGCCCAGCCCTGCTCTGAGCTGGAGGAGCACTGGGGGCATCCTGGATG |
| | TGTACATCTTCCTGGGCCCTGAGCCTAAGTCTGTGGTGCAGCAGTACCTGGATGTGGTGGGCTATCCCTTCATGCCCCCCTACTGGGGCCTGGGCTTTCACCTGTGCAGGTGGGGCTATAGCAGCACTGCCATCACCAGGCAGGT |
| | GGTGGAGAATATGACTAGGGCCCATTTCCCCCTGGATGTGCAGTGGAATGACCTGGATTACATGGACAGCAGGAGGGACTTCACCTTCAATAAGGATGGCTTTAGGGACTTTCCTGCCATGGTGCAGGAGCTGCATCAGGGGGGG |
| | AGGAGGTATATGATGATTGTGGATCCTGCCATCAGCAGCTCTGGCCCTGCTGGCAGCTATAGGCCCTATGATGAGGGCCTGAGGAGGGGGGTGTTCATTACCAATGAGACTGGCCAGCCTCTGATTGGCAAGGTCTGGCCTGGCT |
| | CTACTGCCTTTCCTGACTTCACTAACCCCACTGCCCTGGCCTGGTGGGAGGATATGGTGGCTGAGTTCCATGACCAGGTGCCCTTTGATGGCATGTGGATTGACATGAATGAGCCCTCTAACTTCATCAGGGGCTCTGAGGATGG |
| | CTGCCCCAATAATGAGCTGGAGAACCCCCCCTATGTGCCTGGGGTGGTGGGGGGCACCCTGCAGGCTGCCACCATCTGTGCCAGCAGCCACCAGTTCCTGAGCACCCATTATAATCTGCACAACCTGTATGGCCTGACTGAGGCC |
| | ATTGCCAGCCATAGGGCCCTGGTGAAGGCTAGGGGCACTAGGCCTTTTGTGATTAGCAGGAGCACCTTTGCTGGCCATGGCAGGTATGCTGGCCACTGGACTGGGGATGTGTGGAGCAGCTGGGAGCAGCTGGCCAGCTCTGTGC |
| | CTGAGATTCTGCAGTTCAACCTGCTGGGGGTGCCCCTGGTGGGGGCTGATGTGTGTGGCTTTCTGGGCAACACCTCTGAGGAGCTGTGTGTGAGGTGGACCCAGCTGGGGGCCTTCTACCCCTTTATGAGGAATCACAATAGCCT |
| | GCTGAGCCTGCCCCAGGAGCCCTACTCTTTCTCTGAGCCTGCCCAGCAGGCCATGAGGAAGGCCCTGACTCTGAGGTATGCCCTGCTGCCCCACCTGTATACCCTGTTCCACCAGGCCCATGTGGCTGGGGAGACTGTGGCTAGG |
| | CCTCTGTTCCTGGAGTTCCCCAAGGATAGCTCTACCTGGACTGTGGACCACCAGCTGCTGTGGGGGGAGGCCCTGCTGATCACCCCTGTGCTGCAGGCTGGCAAGGCTGAGGTGACTGGCTACTTTCCCCTGGGCACCTGGTATG |
| | ACCTGCAGACTGTGCCCATTGAGGCCCTGGGCAGCCTGCCTCCCCCCCCAGCTGCCCCAAGGGAGCCTGCCATTCACTCTGAGGGCCAGTGGGTGACCCTGCCTGCCCCCCTGGACACCATCAATGTGCACCTGAGGGCTGGCTA |
| | CATTATCCCCCTGCAGGGCCCTGGCCTGACCACCACTGAATCTAGGCAGCAGCCCATGGCCCTGGCTGTGGCCCTGACCAAGGGGGGGGAGGCCAGGGGGGAGCTGTTTTGGGATGATGGGGAGTCTCTGGAGGTGCTGGAGAGG |
| | GGGGCCTACACTCAGGTGATCTTCCTGGCCAGGAATAATACTATTGTGAATGAGCTGGTGAGGGTGACCTCTGAGGGGGCTGGCCTGCAGCTGCAGAAGGTGACTGTGCTGGGGGTGGCCACTGCCCCCCAGCAGGTGCTGAGCA |
| | ATGGGGTGCCTGTGTCTAACTTTACCTATTCTCCTGACACTAAGGTGCTGGATATCTGTGTGTCTCTGCTGATGGGGGAACAGTTCCTGGTGTCTTGGTGCTGA |
| 9 | > coGAA GAA nucleic acid |
| | ATGGGGGTGAGGCACCCTCCCTGCTCTCACAGGCTGCTGGCTGTGTGTGCCCTGGTGAGCCTGGCCACTGCTGCTCTGCTGGGCCACATCCTGCTGCATGACTTCCTGCTGGTCCCTAGGGAGCTGTCTGGCTCTAGCCCTGTCC |
| | TGGAGGAGACTCACCCAGCCCACCAGCAGGGGGCCAGCAGGCCTGGCCCCAGGGATGCCCAGGCCCACCCTGGCAGGCCTAGGGCTGTGCCCACCCAGTGTGATGTGCCCCCTAACAGCAGGTTTGATTGTGCCCCTGACAAGGC |
| | CATCACCCAGGAGCAGTGTGAGGCCAGGGGCTGCTGCTACATCCCTGCCAAGCAGGGCCTGCAGGGGGCCCAGATGGGGCAGCCCTGGTGCTTCTTCCCCCCCTCTTACCCCAGCTATAAGCTGGAGAACCTGAGCTCTTCTGAG |
| | ATGGGCTACACTGCCACTCTGACCAGGACTACCCCTACTTTCTTCCCCAAGGACATCCTGACCCTGAGGCTGGATGTGATGATGGAGACTGAGAACAGGCTGCACTTTACCATTAAGGATCCTGCCAACAGGAGGTATGAGGTGC |
| | CCCTGGAGACTCCCAGGGTGCATAGCAGGGCCCCCTCTCCCCTGTACTCTGTGGAGTTCTCTGAGGAGCCCTTTGGGGTGATTGTGCACAGGCAGCTGGATGGCAGGGTGCTGCTGAATACCACTGTGGCTCCCCTGTTTTTTGC |
| | TGACCAGTTTCTGCAGCTGAGCACCTCTCTGCCCAGCCAGTATATTACTGGCCTGGCTGAGCATCTGAGCCCCCTGATGCTGTCTACCAGCTGGACCAGGATCACTCTGTGGAACAGGGATCTGGCCCCTACCCCTGGGGCCAAT |
| | CTGTATGGCAGCCACCCTTTCTACCTGGCCCTGGAGGATGGGGGGTCTGCTCATGGGGTGTTCCTGCTGAACAGCAATGCTATGGATGTGGTGCTGCAGCCCTCTCCTGCTCTGAGCTGGAGAAGCACTGGGGGGATCCTGGATG |
| | TGTACATCTTCCTGGGGCCTGAGCCCAAGTCTGTGGTGCAGCAGTACCTGGATGTGGTGGGCTACCCTTTTATGCCTCCTTATTGGGGCCTGGGGTTTCACCTGTGCAGGTGGGGGTACAGCTCTACTGCTATCACCAGGCAGGT |
| | GGTGGAGAACATGACCAGGGCCCACTTCCCCCTGGATGTGCAGTGGAATGATCTGGATTACATGGACTCTAGGAGGGACTTCACCTTCAACAAAGATGGCTTCAGGGACTTCCCTGCCATGGTGCAGGAGCTGCACCAGGGGGGC |
| | AGAAGGTATATGATGATTGTGGATCCTGCTATCAGCAGCTCTGGGCCTGCTGGCTCTTACAGGCCCTATGATGAGGGGCTGAGGAGGGGGGTGTTCATTACTAATGAAACTGGCCAGCCCCTGATTGGCAAGGTGTGGCCTGGCA |
| | GCACTGCTTTCCCTGACTTCACTAACCCTACTGCCCTGGCCTGGTGGGAGGACATGGTGGCTGAGTTCCATGACCAGGTGCCCTTTGATGGCATGTGGATTGACATGAATGAGCCCTCTAATTTTATCAGGGGGTCTGAGGATGG |
| | CTGCCCCAACAATGAGCTGGAGAATCCCCCCTATGTGCCTGGGGTGGTGGGGGGCACCCTGCAGGCTGCCACCATCTGTGCCAGCTCTCACCAGTTTCTGTCTACCCACTATAACCTGCACAACCTGTATGGGCTGACTGAGGCC |
| | ATTGCCAGCCACAGGGCCCTGGTGAAGGCCAGGGGCACCAGGCCCTTTGTGATCAGCAGGTCTACCTTTGCTGGCCATGGGAGGTATGCTGGCCACTGGACTGGGGATGTGTGGAGCAGCTGGGAGCAGCTGGCCAGCTCTGTGC |
| | CTGAGATCCTGCAGTTCAACCTGCTGGGGGTGCCCCTGGTGGGGGCTGATGTGTGTGGCTTCCTGGGCAACACCTCTGAGGAGCTGTGTGTGAGGTGGACCCAGCTGGGGGCTTTTTATCCCTTCATGAGGAACCACAACAGCCT |
| | GCTGAGCCTGCCTCAGGAGCCCTACAGCTTTTCTGAGCCTGCCCAGCAGGCCATGAGAAAGGCCCTGACCCTGAGGTATGCCCTGCTGCCCCACCTGTATACCCTGTTCCACCAGGCTCATGTGGCTGGGGAGACTGTGGCCAGG |
| | CCCCTGTTCCTGGAGTTCCCCAAGGACAGCTCTACCTGGACTGTGGACCACCAGCTGCTGTGGGGGGAGGCCCTGCTGATCACCCCTGTGCTGCAGGCTGGCAAGGCTGAGGTGACTGGCTACTTCCCCCTGGGCACCTGGTATG |
| | ACCTGCAGACTGTGCCCATTGAGGCCCTGGGCAGCCTGCCCCCCCCCCCAGCTGCTCCTAGGGAGCCTGCCATCCACTCTGAGGGCCAGTGGGTGACCCTGCCTGCTCCCCTGGACACCATCAATGTGCACCTGAGGGCTGGCTA |
| | CATCATCCCCCTGCAGGGCCCTGGGCTGACCACCACTGAGTCTAGGCAGCAGCCCATGGCCCTGGCTGTGGCCCTGACCAAGGGGGGGGAGGCCAGGGGGGAGCTGTTCTGGGATGATGGGGAGAGCCTGGAGGTGCTGGAGAGG |
| | GGGGCCTACACTCAGGTGATCTTCCTGGCCAGGAACAATACCATTGTGAATGAGCTGGTGAGGGTGACTTCTGAGGGGGCTGGCCTGCAGCTGCAGAAGGTGACTGTGCTGGGGGTGGCCACTGCCCCTCAGCAGGTGCTGTCTA |
| | ATGGGGTGCCTGTGAGCAACTTCACTTACAGCCCTGACACCAAGGTGCTGGACATCTGTGTGAGCCTGCTGATGGGGGAGCAGTTCCTGGTGTCTTGGTGTTGA |
| 10 | > coGAA GAA nucleic acid |
| | ATGGGGGTGAGGCATCCCCCCTGCTCTCACAGGCTGCTGGCTGTGTGTGCCCTGGTGAGCCTGGCCACTGCTGCCCTGCTGGGCCATATCCTGCTGCATGACTTCCTGCTGGTGCCCAGGGAGCTGTCTGGCTCTTCTCCTGTGC |
| | TGGAGGAGACTCACCCTGCCCACCAGCAGGGGGCCAGCAGGCCTGGCCCTAGGGATGCCCAGGCTCACCCTGGCAGGCCCAGGGCTGTGCCCACTCAGTGTGATGTGCCCCCCAACAGCAGGTTTGACTGTGCCCCTGACAAGGC |
| | TATTACCCAGGAGCAGTGTGAGGCCAGGGGGTGTTGCTACATTCCTGCCAAGCAGGGCCTGCAGGGGGCCCAGATGGGCCAGCCCTGGTGCTTCTTCCCTCCCAGCTACCCCAGCTACAAGCTGGAGAATCTGAGCAGCTCTGAG |
| | ATGGGCTACACTGCCACTCTGACTAGGACTACTCCCACCTTTTTCCCCAAGGATATCCTGACCCTGAGGCTGGATGTGATGATGGAGACTGAGAACAGGCTGCACTTCACCATCAAGGATCCTGCCAACAGGAGGTATGAGGTGC |
| | CCCTGGAGACCCCCAGGGTGCACAGCAGGGCCCCCAGCCCCCTGTACTCTGTGGAGTTCTCTGAGGAGCCCTTTGGGGTGATTGTGCATAGGCAGCTGGATGGCAGGGTGCTGCTGAATACCACTGTGGCTCCCCTGTTCTTTGC |
| | TGATCAGTTCCTGCAGCTGAGCACTAGCCTGCCCTCTCAGTACATCACTGGGCTGGCTGAGCACCTGAGCCCCCTGATGCTGTCTACTTCTTGGACCAGGATCACCCTGTGGAACAGGGACCTGGCCCCCACTCCTGGGGCCAAC |
| | CTGTATGGCAGCCACCCCTTCTACCTGGCCCTGGAGGATGGGGGCTCTGCCCATGGGGTGTTTCTGCTGAACAGCAATGCTATGGATGTGGTGCTGCAGCCTAGCCCTGCTCTGTCTTGGAGGAGCACTGGGGGCATCCTGGATG |
| | TGTATATTTTCCTGGGGCCTGAGCCCAAGTCTGTGGTGCAGCAGTACCTGGATGTGGTGGGGTACCCTTTCATGCCCCCCTATTGGGGCCTGGGCTTCCACCTGTGCAGGTGGGGCTATAGCAGCACTGCCATCACCAGGCAGGT |
| | GGTGGAAAATATGACTAGGGCCCACTTCCCCCTGGATGTGCAGTGGAATGACCTGGACTACATGGATTCTAGGAGGGACTTCACCTTCAACAAGGATGGGTTTAGGGATTTCCCTGCCATGGTGCAGGAGCTGCACCAGGGGGGC |
| | AGGAGGTACATGATGATTGTGGACCCTGCCATCAGCAGCTCTGGCCCAGCTGGCTCTTACAGGCCCTATGATGAGGGCCTGAGGAGAGGGGTGTTCATCACCAATGAAACTGGCCAGCCCCTGATTGGCAAGGTGTGGCCTGGCT |
| | CTACTGCCTTCCCTGATTTCACCAACCCCACTGCCCTGGCCTGGTGGGAGGATATGGTGGCTGAATTCCATGACCAGGTGCCCTTTGATGGCATGTGGATTGACATGAATGAGCCCAGCAACTTTATTAGGGGCTCTGAGGATGG |
| | GTGCCCCAACAATGAGCTGGAGAATCCCCCTTATGTGCCTGGGGTGGTGGGGGGCACTCTGCAGGCTGCCACCATCTGTGCTAGCAGCCACCAGTTCCTGAGCACTCACTACAACCTGCATAACCTGTATGGCCTGACTGAGGCC |
| | ATTGCTTCTCACAGGGCTCTGGTCAAGGCCAGGGGGACCAGACCCTTTGTGATCAGCAGGAGCACCTTTGCTGGCCATGGCAGGTATGCTGGCCACTGGACTGGGGATGTGTGGAGCAGCTGGGAGCAGCTGGCCAGCTCTGTGC |
| | CTGAGATTCTGCAGTTTAACCTGCTGGGGGTGCCCCTGGTGGGGGCTGATGTGTGTGGCTTCCTGGGGAACACCTCTGAGGAGCTGTGTGTGAGGTGGACCCAGCTGGGGGCTTTTTACCCCTTTATGAGGAACCATAATAGCCT |
| | GCTGAGCCTGCCTCAGGAGCCCTACAGCTTCTCTGAGCCTGCCCAGCAGGCCATGAGGAAGGCCCTGACCCTGAGGTATGCCCTGCTGCCCCATCTGTACACCCTGTTTCACCAGGCCCATGTGGCTGGGGAGACTGTGGCCAGA |
| | CCCCTGTTCCTGGAGTTCCCAAAGGACTCTTCTACCTGGACTGTGGACCACCAGCTGCTGTGGGGGGAGGCCCTGCTGATTACCCCTGTGCTGCAGGCTGGGAAGGCTGAGGTGACTGGGTATTTCCCCCTGGGCACCTGGTATG |
| | ACCTGCAGACTGTGCCAATTGAGGCTCTGGGCAGCCTGCCTCCCCCCCCTGCTGCTCCTAGGGAGCCTGCCATTCACTCTGAGGGCCAGTGGGTGACCCTGCCAGCCCCTCTGGACACTATCAATGTGCACCTGAGGGCTGGCTA |
| | CATCATCCCCCTGCAGGGGCCTGGCCTGACTACCACTGAGAGCAGGCAGCAGCCTATGGCTCTGGCTGTGGCCCTGACCAAGGGGGGGGAGGCCAGGGGGGAGCTGTTCTGGGATGATGGGGAGTCTCTGGAGGTGCTGGAGAGG |
| | GGGGCCTACACCCAGGTGATCTTCCTGGCCAGGAATAATACCATTGTGAATGAGCTGGTGAGAGTGACTTCTGAGGGGGCTGGCCTGCAGCTGCAGAAGGTGACTGTGCTGGGGGTGGCCACTGCCCCCCAGCAGGTCCTGTCTA |
| | ATGGGGTGCCTGTGTCTAACTTTACCTACAGCCCTGACACCAAGGTGCTGGACATCTGTGTGAGCCTGCTGATGGGGGAGCAGTTCCTGGTGAGCTGGTGTTGA |
| 11 | > coGAA GAA nucleic acid |
| | ATGGGGGTGAGGCACCCCCCTTGCTCTCACAGGCTGCTGGCTGTCTGTGCCCTGGTGAGCCTGGCCACTGCTGCTCTGCTGGGGCACATCCTGCTGCATGATTTTCTGCTGGTGCCCAGGGAGCTGTCTGGGAGCTCTCCAGTGC |
| | TGGAGGAGACCCACCCTGCCCACCAGCAGGGGGCCAGCAGACCTGGGCCCAGGGATGCTCAGGCTCACCCTGGCAGGCCCAGGGCTGTGCCCACTCAGTGTGATGTGCCCCCCAACAGCAGGTTTGACTGTGCCCCTGACAAGGC |
| | CATCACCCAGGAGCAGTGTGAGGCTAGGGGCTGCTGTTACATCCCTGCCAAGCAGGGCCTGCAGGGGGCCCAGATGGGCCAGCCCTGGTGCTTCTTCCCCCCCAGCTACCCCAGCTACAAGCTGGAGAACCTGTCTAGCTCTGAG |
| | ATGGGCTATACTGCTACCCTGACTAGGACCACCCCTACTTTCTTCCCCAAGGACATCCTGACCCTGAGGCTGGATGTGATGATGGAGACTGAGAATAGGCTGCATTTCACCATCAAGGACCCTGCTAACAGGAGGTATGAAGTGC |
| | CCCTGGAGACCCCCAGGGTGCATAGCAGGGCCCCCTCTCCTCTGTACTCTGTGGAGTTCTCTGAAGAGCCCTTTGGGGTGATTGTGCATAGGCAGCTGGATGGCAGGGTGCTGCTGAACACTACTGTGGCCCCCCTGTTTTTTGC |
| | TGACCAGTTCCTGCAGCTGAGCACCAGCCTGCCCAGCCAGTACATCACTGGCCTGGCTGAGCACCTGAGCCCACTGATGCTGAGCACCAGCTGGACTAGGATTACCCTGTGGAACAGGGACCTGGCCCCCACTCCTGGGGCCAAC |
| | CTGTATGGCAGCCACCCCTTCTACCTGGCCCTGGAGGATGGGGGCTCTGCCCATGGGGTGTTCCTGCTGAACAGCAATGCCATGGATGTGGTGCTGCAGCCCTCTCCTGCCCTGTCTTGGAGGAGCACTGGGGGGATTCTGGATG |
| | TGTACATCTTTCTGGGGCCTGAGCCCAAGTCTGTGGTGCAGCAGTACCTGGATGTGGTGGGGTACCCCTTTATGCCTCCCTATTGGGGCCTGGGGTTCCATCTGTGCAGGTGGGGGTACTCTAGCACTGCTATCACCAGGCAGGT |
| | GGTGGAGAACATGACCAGGGCCCATTTCCCCCTGGATGTGCAGTGGAATGACCTGGATTACATGGACTCTAGAAGGGACTTCACCTTCAACAAGGATGGCTTCAGGGATTTCCCTGCTATGGTGCAGGAGCTGCATCAGGGGGGC |
| | AGGAGGTACATGATGATTGTGGATCCTGCCATCAGCAGCTCTGGGCCTGCTGGGAGCTATAGGCCTTATGATGAGGGCCTGAGGAGGGGGGTGTTTATTACCAATGAGACTGGCCAGCCCCTGATTGGCAAGGTGTGGCCAGGCA |
| | GCACTGCCTTCCCTGACTTCACTAACCCTACTGCTCTGGCCTGGTGGGAGGACATGGTGGCTGAATTTCATGACCAGGTGCCCTTTGATGGGATGTGGATTGACATGAATGAGCCTTCTAATTTCATCAGGGGCTCTGAGGATGG |
| | GTGCCCCAACAATGAGCTGGAGAACCCCCCCTATGTGCCTGGGGTGGTGGGGGGCACCCTGCAGGCTGCCACTATCTGTGCTAGCAGCCATCAGTTCCTGAGCACTCACTACAATCTGCACAACCTGTATGGCCTGACTGAGGCC |
| | ATTGCCAGCCATAGGGCCCTGGTGAAGGCTAGGGGCACTAGGCCCTTTGTGATCAGCAGGAGCACCTTTGCTGGCCATGGGAGGTATGCTGGCCACTGGACTGGGGATGTGTGGAGCAGCTGGGAGCAGCTGGCCAGCTCTGTGC |
| | CTGAGATCCTGCAGTTCAACCTGCTGGGGGTGCCCCTGGTGGGGGCTGATGTGTGTGGCTTCCTGGGGAACACCTCTGAAGAGCTGTGTGTGAGGTGGACCCAGCTGGGGGCCTTCTACCCCTTCATGAGGAATCACAACAGCCT |
| | GCTGAGCCTGCCTCAGGAGCCCTACTCTTTCTCTGAGCCTGCCCAGCAGGCCATGAGGAAGGCTCTGACCCTGAGGTATGCCCTGCTGCCTCACCTGTACACCCTGTTTCATCAGGCCCATGTGGCTGGGGAGACTGTGGCCAGG |
| | CCCCTGTTCCTGGAGTTCCCCAAGGATAGCAGCACCTGGACTGTGGACCACCAGCTGCTGTGGGGGGAAGCCCTGCTGATCACCCCTGTGCTGCAGGCTGGCAAGGCTGAGGTGACTGGCTACTTCCCCCTGGGCACCTGGTATG |
| | ACCTGCAGACTGTGCCCATTGAGGCCCTGGGCTCTCTGCCCCCTCCCCCTGCTGCCCCCAGGGAGCCTGCCATCCACTCTGAGGGCCAGTGGGTGACTCTGCCTGCCCCTCTGGATACCATTAATGTGCACCTGAGGGCTGGGTA |
| | CATCATCCCTCTGCAGGGCCCTGGCCTGACTACCACTGAGAGCAGGCAGCAGCCTATGGCCCTGGCTGTGGCTCTGACCAAGGGGGGGGAGGCCAGGGGGGAGCTGTTCTGGGATGATGGGGAGAGCCTGGAGGTGCTGGAGAGG |
| | GGGGCTTATACCCAGGTGATCTTCCTGGCCAGGAACAACACCATTGTGAATGAGCTGGTGAGGGTGACCTCTGAGGGGGCTGGCCTGCAGCTGCAGAAGGTGACTGTGCTGGGGGTGGCCACTGCCCCTCAGCAGGTGCTGAGCA |
| | ATGGGGTGCCTGTGAGCAATTTCACTTACAGCCCTGACACCAAGGTGCTGGATATTTGTGTGAGCCTGCTGATGGGGGAGCAGTTCCTGGTGTCTTGGTGCTAA |
| 12 | > coGAA GAA nucleic acid |
| | ATGGGGGTGAGGCATCCCCCCTGCAGCCATAGGCTGCTGGCTGTGTGTGCCCTGGTGAGCCTGGCTACTGCTGCCCTGCTGGGGCACATCCTGCTGCATGACTTCCTGCTGGTGCCCAGGGAACTGTCTGGCAGCAGCCCTGTGC |
| | TGGAGGAGACTCATCCTGCTCACCAGCAGGGGGCCTCCAGGCCTGGCCCCAGGGATGCCCAGGCTCACCCTGGCAGGCCCAGGGCTGTGCCCACTCAGTGTGATGTGCCCCCCAACAGCAGGTTTGACTGTGCCCCTGACAAGGC |
| | CATCACTCAGGAGCAGTGTGAGGCCAGGGGCTGCTGCTATATCCCTGCCAAGCAGGGGCTGCAGGGGGCCCAGATGGGGCAGCCTTGGTGCTTTTTCCCCCCCAGCTACCCCAGCTACAAGCTGGAGAACCTGTCTAGCTCTGAG |
| | ATGGGCTATACTGCCACTCTGACCAGGACCACCCCCACCTTCTTCCCCAAAGACATTCTGACCCTGAGGCTGGATGTGATGATGGAGACTGAGAATAGGCTGCACTTCACCATCAAGGACCCTGCTAACAGGAGGTATGAGGTGC |
| | CCCTGGAGACTCCTAGGGTGCACAGCAGGGCCCCCAGCCCCCTGTATTCTGTGGAGTTCTCTGAGGAGCCCTTTGGGGTGATTGTGCATAGGCAGCTGGATGGGAGGGTGCTGCTGAACACTACTGTGGCTCCCCTGTTCTTTGC |
| | TGACCAGTTCCTGCAGCTGAGCACCAGCCTGCCCTCTCAGTACATCACTGGCCTGGCTGAGCACCTGAGCCCCCTGATGCTGAGCACCAGCTGGACCAGGATCACTCTGTGGAACAGAGACCTGGCCCCCACCCCTGGGGCCAAC |
| | CTGTATGGCAGCCATCCCTTCTACCTGGCTCTGGAGGATGGGGGGTCTGCCCATGGGGTGTTTCTGCTGAACAGCAATGCCATGGATGTGGTGCTGCAGCCCAGCCCTGCCCTGAGCTGGAGGAGCACTGGGGGCATCCTGGATG |
| | TGTACATTTTCCTGGGCCCTGAACCCAAGTCTGTGGTGCAGCAGTACCTGGATGTGGTGGGGTATCCCTTCATGCCTCCTTACTGGGGCCTGGGCTTCCATCTGTGCAGGTGGGGCTACAGCAGCACTGCTATCACCAGGCAGGT |
| | GGTGGAGAACATGACCAGGGCCCATTTCCCCCTGGATGTGCAGTGGAATGATCTGGACTACATGGACTCTAGGAGGGACTTCACTTTCAACAAGGATGGCTTCAGAGATTTCCCTGCCATGGTGCAGGAGCTGCACCAGGGGGGC |
| | AGAAGGTACATGATGATTGTGGACCCTGCCATCTCTAGCTCTGGCCCTGCTGGGAGCTACAGGCCCTATGATGAGGGGCTGAGGAGGGGGGTGTTTATCACCAATGAGACTGGCCAGCCCCTGATTGGCAAGGTGTGGCCTGGCA |
| | GCACTGCCTTCCCTGACTTCACCAATCCCACTGCCCTGGCCTGGTGGGAGGATATGGTGGCTGAGTTCCATGACCAGGTGCCCTTTGATGGGATGTGGATTGACATGAATGAGCCCAGCAACTTTATCAGGGGCTCTGAGGATGG |
| | CTGCCCCAACAATGAACTGGAAAACCCCCCCTATGTGCCTGGGGTGGTGGGGGGCACTCTGCAGGCTGCCACTATCTGTGCCAGCAGCCACCAGTTCCTGAGCACCCACTACAACCTGCACAATCTGTATGGCCTGACTGAGGCC |
| | ATTGCCAGCCACAGGGCCCTGGTGAAGGCTAGGGGCACCAGGCCCTTTGTGATCAGCAGGAGCACCTTTGCTGGCCATGGCAGGTATGCTGGCCACTGGACTGGGGATGTGTGGAGCTCTTGGGAGCAGCTGGCCAGCTCTGTGC |
| | CTGAGATCCTGCAGTTCAACCTGCTGGGGGTGCCCCTGGTGGGGGCTGATGTGTGTGGCTTTCTGGGCAATACTTCTGAGGAGCTGTGTGTGAGGTGGACCCAGCTGGGGGCCTTTTACCCCTTCATGAGGAACCACAACTCTCT |
| | GCTGAGCCTGCCCCAGGAACCCTATAGCTTTTCTGAGCCTGCCCAGCAGGCCATGAGGAAGGCCCTGACTCTGAGGTATGCTCTGCTGCCCCACCTGTACACCCTGTTCCACCAGGCCCATGTGGCTGGGGAGACTGTGGCCAGG |
| | CCCCTGTTCCTGGAGTTCCCCAAGGACAGCAGCACTTGGACTGTGGACCACCAGCTGCTGTGGGGGGAGGCTCTGCTGATCACTCCTGTGCTGCAGGCTGGCAAGGCTGAGGTGACTGGCTACTTCCCCCTGGGGACTTGGTATG |
| | ACCTGCAGACTGTGCCTATTGAGGCCCTGGGGAGCCTGCCTCCCCCCCCTGCTGCTCCCAGGGAGCCTGCCATCCACTCTGAGGGGCAGTGGGTGACTCTGCCTGCTCCCCTGGACACTATCAATGTGCACCTGAGGGCTGGGTA |
| | CATCATCCCCCTGCAGGGCCCTGGGCTGACCACCACTGAGTCTAGGCAGCAGCCCATGGCTCTGGCTGTGGCTCTGACCAAGGGGGGGGAGGCCAGGGGGGAGCTGTTCTGGGATGATGGGGAGAGCCTGGAGGTGCTGGAGAGG |
| | GGGGCCTACACCCAGGTGATCTTCCTGGCTAGAAACAACACCATTGTGAATGAGCTGGTGAGGGTGACTTCTGAGGGGGCTGGGCTGCAGCTGCAGAAGGTGACTGTGCTGGGGGTGGCTACTGCCCCACAGCAGGTGCTGTCTA |
| | ATGGGGTGCCAGTGTCTAACTTCACCTATAGCCCTGATACTAAGGTGCTGGACATCTGTGTGTCTCTGCTGATGGGGGAGCAGTTCCTGGTGTCTTGGTGCTGA |
| 13 | > coGAA GAA nucleic acid |
| | ATGGGGGTGAGGCATCCTCCCTGCAGCCATAGGCTGCTGGCTGTGTGTGCCCTGGTGAGCCTGGCTACTGCTGCTCTGCTGGGGCACATCCTGCTGCATGATTTCCTGCTGGTGCCTAGGGAGCTGTCTGGCAGCAGCCCTGTCC |
| | TGGAGGAGACCCACCCTGCCCATCAGCAGGGGGCCTCTAGACCTGGCCCCAGGGATGCCCAGGCCCATCCTGGGAGGCCCAGGGCTGTGCCCACCCAGTGTGATGTGCCTCCCAACAGCAGGTTTGACTGTGCTCCTGACAAAGC |
| | CATCACCCAGGAGCAGTGTGAAGCCAGGGGCTGTTGTTACATTCCTGCCAAGCAGGGCCTGCAGGGGGCCCAGATGGGCCAGCCCTGGTGCTTCTTCCCCCCTAGCTACCCCAGCTACAAGCTGGAGAATCTGAGCAGCTCTGAA |
| | ATGGGCTACACTGCCACCCTGACTAGGACCACCCCCACCTTCTTCCCTAAGGATATCCTGACCCTGAGGCTGGATGTGATGATGGAGACTGAGAACAGGCTGCACTTCACCATCAAGGATCCTGCCAACAGGAGGTATGAGGTGC |
| | CCCTGGAGACCCCCAGGGTGCACAGCAGGGCTCCCTCTCCCCTGTATTCTGTGGAATTCTCTGAGGAGCCCTTTGGGGTGATTGTGCACAGGCAGCTGGATGGCAGAGTGCTGCTGAACACTACTGTGGCTCCCCTGTTCTTTGC |
| | TGATCAGTTCCTGCAGCTGAGCACTTCTCTGCCCAGCCAGTACATCACTGGGCTGGCTGAGCACCTGAGCCCTCTGATGCTGAGCACCAGCTGGACCAGGATCACCCTGTGGAACAGGGACCTGGCTCCCACCCCTGGGGCCAAT |
| | CTGTATGGCAGCCACCCCTTCTACCTGGCTCTGGAGGATGGGGGCTCTGCCCATGGGGTGTTCCTGCTGAACTCTAATGCCATGGATGTGGTGCTGCAGCCCAGCCCTGCCCTGAGCTGGAGGAGCACTGGGGGCATCCTGGATG |
| | TGTACATCTTCCTGGGCCCTGAGCCCAAGTCTGTGGTCCAGCAGTATCTGGATGTGGTGGGGTACCCTTTCATGCCCCCCTACTGGGGCCTGGGCTTCCACCTGTGCAGGTGGGGGTACAGCAGCACTGCCATCACCAGGCAGGT |
| | GGTGGAGAATATGACCAGGGCCCACTTCCCCCTGGATGTGCAGTGGAATGATCTGGACTACATGGATTCTAGGAGGGACTTCACCTTCAACAAGGATGGCTTCAGGGACTTTCCTGCCATGGTGCAGGAGCTGCACCAGGGGGGC |
| | AGGAGGTACATGATGATTGTGGACCCAGCCATCTCTAGCTCTGGGCCAGCTGGCAGCTACAGGCCCTATGATGAGGGCCTGAGGAGGGGGGTGTTCATCACTAATGAGACTGGCCAGCCCCTGATTGGCAAGGTGTGGCCTGGGA |
| | GCACTGCCTTCCCTGACTTCACCAACCCCACTGCCCTGGCCTGGTGGGAGGATATGGTGGCTGAGTTCCATGACCAGGTGCCCTTTGATGGGATGTGGATTGACATGAATGAGCCCTCTAACTTTATTAGGGGCTCTGAGGATGG |
| | GTGCCCTAACAATGAGCTGGAGAACCCCCCTTATGTGCCTGGGGTGGTGGGGGGCACCCTGCAGGCTGCCACCATCTGTGCCAGCAGCCACCAGTTCCTGTCTACCCACTACAACCTGCACAATCTGTATGGGCTGACTGAGGCT |
| | ATTGCCAGCCACAGGGCCCTGGTGAAGGCTAGGGGGACCAGGCCTTTTGTGATCAGCAGGAGCACCTTTGCTGGGCATGGCAGATATGCTGGCCATTGGACTGGGGATGTGTGGTCTAGCTGGGAGCAGCTGGCCAGCTCTGTGC |
| | CTGAGATCCTGCAGTTCAATCTGCTGGGGGTGCCCCTGGTGGGGGCTGATGTGTGTGGGTTCCTGGGCAATACCTCTGAGGAGCTGTGTGTGAGGTGGACCCAGCTGGGGGCCTTCTACCCCTTCATGAGGAACCACAACAGCCT |
| | GCTGAGCCTGCCTCAGGAGCCCTATTCTTTTTCTGAGCCTGCCCAGCAGGCTATGAGGAAGGCCCTGACTCTGAGGTATGCCCTGCTGCCTCACCTGTACACCCTGTTCCACCAGGCCCATGTGGCTGGGGAGACTGTGGCCAGG |
| | CCCCTGTTCCTGGAGTTCCCCAAGGATAGCTCTACCTGGACTGTGGACCATCAGCTGCTGTGGGGGGAGGCCCTGCTGATCACCCCTGTGCTGCAGGCTGGCAAGGCTGAGGTGACTGGCTACTTTCCTCTGGGCACCTGGTATG |
| | ATCTGCAGACTGTGCCCATTGAAGCCCTGGGCAGCCTGCCTCCTCCCCCTGCTGCCCCTAGGGAGCCTGCCATCCACTCTGAGGGCCAGTGGGTGACCCTGCCAGCCCCTCTGGACACCATCAATGTGCATCTGAGGGCTGGCTA |
| | CATCATTCCCCTGCAGGGCCCTGGGCTGACCACCACTGAGAGCAGGCAGCAGCCCATGGCTCTGGCTGTGGCCCTGACCAAGGGGGGGGAGGCCAGGGGGGAGCTGTTCTGGGATGATGGGGAGAGCCTGGAGGTGCTGGAGAGG |
| | GGGGCCTACACCCAGGTGATCTTCCTGGCCAGGAATAATACCATTGTGAATGAGCTGGTGAGGGTGACTTCTGAGGGGGCTGGCCTGCAGCTGCAGAAGGTGACTGTGCTGGGGGTGGCTACTGCTCCTCAGCAGGTGCTGTCTA |
| | ATGGGGTGCCTGTGAGCAACTTCACTTACAGCCCTGACACTAAGGTGCTGGACATTTGTGTGAGCCTGCTGATGGGGGAGCAGTTCCTGGTGTCTTGGTGCTGA |
| 14 | > coGAAAA GAA nucleic acid |
| | ATGGGAGTGAGGCACCCCCCCTGCTCCCATAGACTCCTGGCTGTCTGTGCCCTGGTGTCCTTGGCAACAGCTGCACTCCTGGGGCACATCCTACTCCATGATTTCCTGCTGGTTCCCAGAGAGCTGAGTGGCTCCTCCCCAGTCC |
| | TGGAGGAGACTCACCCAGCTCACCAGCAGGGAGCCAGCAGACCAGGGCCCAGAGATGCCCAGGCACACCCTGGCAGACCCAGAGCAGTGCCCACACAGTGTGATGTCCCCCCCAACAGCAGATTTGATTGTGCCCCTGACAAGGC |
| | CATAACCCAGGAACAGTGTGAGGCCAGAGGCTGCTGCTACATCCCTGCAAAGCAGGGGCTGCAGGGAGCCCAGATGGGGCAGCCCTGGTGCTTCTTCCCACCCAGCTACCCCAGCTACAAGCTGGAGAACCTGAGCTCCTCTGAA |
| | ATGGGCTACACAGCCACCCTGACCAGAACCACCCCCACCTTCTTCCCCAAGGACATCCTGACCCTGAGACTGGATGTGATGATGGAGACTGAGAACAGACTCCACTTCACCATCAAAGATCCAGCTAACAGGAGATATGAGGTGC |
| | CCTTAGAGACCCCTAGAGTCCACAGCAGAGCACCTTCCCCACTCTACTCTGTGGAGTTCTCTGAGGAGCCCTTTGGGGTGATTGTGCACAGACAGCTGGATGGCAGAGTGCTGCTGAACACAACAGTGGCTCCCCTGTTCTTTGC |
| | TGACCAGTTCCTTCAGCTGTCCACCTCTCTGCCCTCTCAGTATATCACAGGCCTGGCTGAGCACCTCAGTCCCCTGATGCTCAGCACCAGCTGGACCAGGATCACCCTGTGGAACAGAGACCTTGCTCCCACACCTGGTGCCAAC |
| | CTCTATGGGTCTCACCCTTTCTACCTAGCTCTGGAGGATGGAGGGTCTGCACATGGGGTGTTCCTGCTAAACAGCAATGCCATGGATGTAGTCCTGCAGCCCAGCCCTGCCCTTAGCTGGAGGTCTACAGGTGGGATCCTGGATG |
| | TCTACATCTTCCTAGGCCCAGAGCCCAAGTCTGTGGTGCAGCAGTACCTGGATGTGGTAGGATACCCCTTCATGCCTCCATACTGGGGCCTGGGCTTCCACCTGTGCAGATGGGGCTACTCCTCCACAGCTATCACTAGACAGGT |
| | GGTGGAGAACATGACCAGGGCCCACTTCCCCCTGGATGTCCAGTGGAATGATCTAGACTACATGGACTCCAGAAGGGACTTCACCTTCAACAAGGATGGCTTCAGAGACTTCCCTGCCATGGTGCAGGAGCTGCACCAGGGAGGA |
| | AGAAGATACATGATGATTGTGGATCCTGCCATCAGCAGCTCTGGCCCTGCTGGGAGCTACAGGCCCTATGATGAGGGTCTGAGAAGGGGGGTTTTCATCACCAATGAGACAGGCCAGCCTCTGATTGGGAAGGTGTGGCCAGGGT |
| | CCACTGCCTTCCCAGACTTCACCAACCCCACAGCCCTGGCCTGGTGGGAGGACATGGTGGCTGAGTTCCATGACCAGGTGCCCTTTGATGGCATGTGGATTGACATGAATGAGCCTTCCAACTTCATAAGAGGCTCTGAGGATGG |
| | CTGCCCCAACAATGAGCTAGAGAACCCACCCTATGTGCCTGGGGTAGTTGGGGGGACCCTCCAGGCTGCCACCATCTGTGCCTCCAGCCACCAGTTTCTCTCCACACACTACAACCTGCACAACCTCTATGGCCTGACAGAAGCC |
| | ATTGCCTCCCACAGGGCTCTGGTGAAGGCTAGAGGGACAAGACCATTTGTGATCTCCAGATCTACCTTTGCTGGCCATGGCAGATATGCTGGCCACTGGACAGGGGATGTGTGGAGCTCCTGGGAGCAGCTAGCCTCCTCTGTGC |
| | CAGAAATCCTGCAGTTTAACCTGCTGGGGGTGCCTCTGGTTGGGGCTGATGTCTGTGGCTTCCTGGGCAACACCTCAGAGGAGCTGTGTGTGAGATGGACCCAGCTGGGGGCCTTCTACCCCTTCATGAGAAACCATAACAGCCT |
| | GCTCAGTCTGCCCCAGGAGCCTTACAGCTTCTCTGAGCCTGCCCAGCAGGCCATGAGGAAGGCCCTCACCCTGAGATATGCACTCCTCCCCCACCTCTACACACTGTTCCACCAGGCCCATGTAGCAGGGGAGACAGTGGCCAGA |
| | CCCCTCTTCCTGGAGTTCCCCAAGGACTCTAGCACCTGGACTGTGGACCACCAGCTCCTGTGGGGGGAGGCCCTGCTCATCACCCCAGTGCTCCAGGCTGGGAAGGCTGAAGTGACTGGCTACTTCCCCTTGGGCACATGGTATG |
| | ACCTGCAGACAGTGCCAATAGAGGCCCTTGGCAGCCTCCCACCCCCACCTGCAGCTCCCAGAGAGCCAGCCATCCACTCTGAGGGGCAGTGGGTGACACTGCCAGCCCCCCTGGACACCATCAATGTGCACCTAAGAGCTGGGTA |
| | CATCATCCCCCTGCAGGGCCCTGGCCTCACAACCACAGAGTCCAGACAGCAGCCCATGGCCCTGGCTGTGGCCCTGACCAAGGGTGGAGAGGCCAGAGGGGAGCTGTTCTGGGATGATGGAGAGAGCCTAGAAGTGCTGGAGAGA |
| | GGGGCCTACACACAGGTCATCTTCCTGGCCAGGAATAACACAATTGTGAATGAGCTGGTAAGAGTGACCAGTGAGGGAGCTGGCCTGCAGCTGCAGAAGGTGACTGTCCTGGGAGTGGCCACAGCACCCCAGCAGGTCCTCTCCA |
| | ATGGTGTCCCTGTCTCCAACTTCACCTACAGCCCTGACACCAAGGTCCTAGACATCTGTGTCAGTCTGTTGATGGGAGAGCAGTTTCTGGTCAGCTGGTGT |
| 15 | > coGAAAA GAA nucleic acid |
| | ATGGGTGTTAGGCATCCACCATGTTCTCATAGGCTTTTAGCTGTGTGTGCTTTGGTATCACTGGCAACTGCTGCACTTTTAGGTCATATTCTACTACATGATTTTCTACTAGTACCAAGGGAGTTATCTGGTTCTTCTCCAGTTT |
| | TGGAGGAGACTCATCCTGCTCATCAGCAGGGTGCATCAAGGCCAGGTCCTAGAGATGCACAGGCACATCCTGGTAGGCCAAGGGCAGTTCCAACACAGTGTGATGTGCCACCAAATTCAAGGTTTGATTGTGCTCCTGATAAAGC |
| | TATAACACAGGAACAGTGTGAGGCTAGGGGATGTTGTTATATACCAGCTAAACAAGGTCTTCAGGGAGCTCAAATGGGACAGCCTTGGTGTTTTTTTCCTCCTTCATATCCTTCATATAAATTGGAAAATCTGAGTTCATCTGAG |
| | ATGGGTTATACTGCAACATTGACTAGAACTACACCAACATTTTTTCCTAAAGATATATTGACACTAAGATTAGATGTGATGATGGAAACTGAAAATAGACTTCATTTTACAATTAAAGATCCAGCTAATAGAAGGTATGAGGTTC |
| | CATTAGAGACACCTAGGGTGCATTCTAGAGCTCCTAGTCCTTTGTATAGTGTTGAGTTTAGTGAGGAACCATTTGGTGTTATAGTGCATAGACAGCTGGATGGTAGAGTTCTTCTTAATACAACAGTGGCTCCTCTATTTTTTGC |
| | TGATCAATTTTTGCAATTGAGTACTTCTTTGCCATCTCAGTATATAACTGGTTTAGCAGAACATTTGAGTCCTTTGATGCTTTCTACATCTTGGACAAGGATAACATTATGGAATAGGGATCTTGCTCCTACACCTGGTGCAAAT |
| | CTGTATGGTTCTCATCCATTTTATCTTGCTTTAGAGGATGGTGGAAGTGCTCATGGTGTGTTTTTGTTGAATTCTAATGCTATGGATGTAGTGCTTCAACCATCACCAGCTCTTAGTTGGAGATCAACAGGTGGAATACTGGATG |
| | TATATATATTTTTAGGTCCAGAACCTAAATCTGTGGTGCAGCAATATTTAGATGTAGTGGGTTATCCTTTTATGCCACCTTATTGGGGTCTTGGATTTCATCTTTGTAGGTGGGGTTATTCTTCAACTGCAATTACAAGACAGGT |
| | AGTGGAGAATATGACTAGAGCTCATTTTCCACTGGATGTGCAGTGGAATGATTTAGATTATATGGATTCTAGGAGGGATTTTACTTTTAATAAAGATGGTTTTAGAGATTTTCCTGCAATGGTGCAGGAACTTCATCAGGGTGGT |
| | AGAAGGTATATGATGATTGTGGATCCTGCTATATCAAGTTCTGGTCCTGCAGGAAGTTATAGGCCTTATGATGAAGGTCTTAGGAGGGGTGTTTTTATAACAAATGAGACTGGTCAGCCTCTGATTGGAAAAGTGTGGCCTGGAT |
| | CAACAGCTTTTCCTGATTTTACAAATCCTACTGCTTTAGCTTGGTGGGAGGATATGGTTGCAGAATTTCATGATCAGGTGCCTTTTGATGGTATGTGGATAGATATGAATGAACCATCTAATTTTATAAGAGGATCTGAGGATGG |
| | TTGTCCTAATAATGAACTGGAGAATCCTCCATATGTTCCTGGTGTAGTGGGTGGAACATTGCAGGCTGCAACTATATGTGCATCATCACATCAGTTTCTAAGTACTCATTATAATCTTCATAATTTGTATGGTTTGACAGAAGCA |
| | ATAGCATCTCATAGAGCATTGGTGAAAGCTAGGGGTACAAGACCATTTGTGATTTCAAGGTCTACATTTGCAGGACATGGTAGATATGCTGGACATTGGACAGGAGATGTGTGGAGTTCATGGGAGCAGCTAGCAAGTAGTGTGC |
| | CAGAAATATTGCAGTTTAATCTGTTAGGAGTTCCACTTGTGGGTGCTGATGTTTGTGGTTTTTTAGGTAATACAAGTGAAGAGCTGTGTGTGAGATGGACTCAGCTTGGTGCTTTTTATCCATTTATGAGAAATCATAATAGTCT |
| | ACTTTCTCTTCCACAGGAACCATATAGTTTTTCTGAGCCTGCTCAGCAAGCAATGAGGAAAGCACTTACTCTGAGGTATGCTCTTCTGCCACATCTTTATACACTTTTTCATCAGGCACATGTAGCTGGAGAGACAGTGGCAAGG |
| | CCACTATTTTTGGAATTTCCTAAAGATTCAAGTACATGGACAGTGGATCATCAGTTGTTGTGGGGTGAAGCTTTACTTATTACACCAGTTCTGCAAGCAGGTAAAGCAGAGGTGACAGGTTATTTTCCACTGGGTACTTGGTATG |
| | ATCTGCAGACAGTGCCAATAGAAGCTCTGGGTAGTTTGCCACCTCCACCTGCTGCTCCTAGAGAACCTGCTATACATAGTGAGGGTCAGTGGGTAACACTACCAGCACCATTGGATACAATTAATGTGCATTTGAGGGCTGGATA |
| | TATAATACCTTTGCAAGGTCCTGGTTTAACAACTACAGAGTCTAGGCAGCAGCCAATGGCACTTGCTGTGGCACTGACAAAAGGTGGAGAAGCAAGGGGTGAGCTATTTTGGGATGATGGAGAATCTCTTGAAGTGTTAGAAAGG |
| | GGTGCATATACTCAGGTAATTTTTTTGGCTAGGAATAATACAATAGTGAATGAGCTGGTAAGAGTAACATCTGAAGGAGCAGGTCTGCAGTTACAGAAAGTAACTGTGCTAGGAGTAGCTACTGCACCACAGCAGGTATTGTCTA |
| | ATGGAGTACCTGTGTCTAATTTTACTTATAGTCCAGATACAAAAGTGTTAGATATTTGTGTGTCTTTGCTGATGGGAGAGCAGTTTCTTGTGTCATGGTGT |
| 16 | > coGAA GAA nucleic acid |
| | ATGGGAGTTAGGCATCCTCCATGTTCTCATAGGCTGTTGGCAGTATGTGCTCTTGTGTCACTGGCTACTGCTGCTTTACTGGGGCATATACTGCTGCATGATTTTTTACTTGTGCCTAGGGAGCTGTCTGGATCTTCTCCTGTAC |
| | TAGAGGAGACACATCCTGCTCATCAGCAAGGTGCATCAAGGCCAGGACCAAGAGATGCTCAGGCTCATCCTGGAAGGCCTAGAGCAGTGCCAACACAATGTGATGTTCCTCCTAATTCAAGATTTGATTGTGCACCTGATAAAGC |
| | TATAACACAAGAACAGTGTGAGGCTAGGGGTTGTTGTTATATTCCTGCAAAACAAGGGTTGCAAGGGGCACAGATGGGACAACCTTGGTGTTTCTTCCCACCATCTTATCCTAGTTATAAATTGGAGAATTTGTCTTCAAGTGAA |
| | ATGGGGTATACAGCTACATTGACAAGAACAACTCCAACATTTTTTCCAAAGGATATTCTTACTTTGAGGCTAGATGTGATGATGGAGACAGAGAATAGGTTGCATTTTACTATAAAAGATCCAGCTAATAGAAGATATGAGGTTC |
| | CTTTAGAGACTCCAAGGGTGCATTCTAGAGCTCCTTCTCCTTTGTATAGTGTGGAGTTTTCAGAGGAACCATTTGGTGTTATTGTTCATAGACAGCTGGATGGTAGGGTGCTGTTGAATACTACAGTAGCTCCTCTGTTTTTTGC |
| | TGATCAGTTTTTACAGTTGTCAACTAGTCTTCCAAGTCAGTATATTACAGGTTTGGCAGAGCATCTATCTCCATTGATGCTGTCTACAAGTTGGACAAGAATAACACTTTGGAATAGAGATCTAGCACCTACACCTGGAGCTAAT |
| | TTGTATGGATCACATCCATTTTATTTGGCATTGGAAGATGGAGGGTCTGCTCATGGAGTTTTTTTATTGAATAGTAATGCTATGGATGTAGTGCTGCAGCCTTCTCCAGCACTAAGTTGGAGATCTACTGGTGGAATTCTTGATG |
| | TTTATATATTTTTAGGGCCTGAACCTAAGTCTGTGGTGCAGCAATATTTAGATGTAGTTGGATATCCTTTTATGCCTCCTTATTGGGGACTTGGTTTTCATCTTTGTAGATGGGGTTATAGTTCTACTGCAATAACAAGGCAGGT |
| | GGTGGAGAATATGACAAGAGCACATTTTCCACTTGATGTGCAATGGAATGATTTAGATTATATGGATTCTAGAAGAGATTTTACTTTTAATAAGGATGGGTTTAGAGATTTTCCAGCTATGGTACAGGAATTGCATCAGGGTGGA |
| | AGAAGGTATATGATGATAGTGGATCCAGCTATTTCTAGTTCTGGTCCTGCTGGGTCTTATAGGCCATATGATGAGGGGCTGAGAAGAGGAGTGTTTATTACAAATGAAACAGGGCAACCTCTAATTGGGAAAGTGTGGCCTGGGA |
| | GTACTGCTTTTCCAGATTTTACAAATCCAACAGCACTTGCATGGTGGGAGGATATGGTGGCAGAGTTTCATGATCAAGTTCCTTTTGATGGGATGTGGATAGATATGAATGAGCCATCAAATTTTATAAGGGGTTCTGAGGATGG |
| | TTGTCCAAATAATGAGTTGGAGAATCCACCTTATGTGCCAGGTGTAGTGGGTGGAACACTGCAGGCAGCAACTATTTGTGCATCATCTCATCAGTTTCTTAGTACTCATTATAATCTACATAATTTGTATGGTTTGACAGAGGCA |
| | ATAGCTTCTCATAGGGCTTTGGTGAAGGCTAGAGGTACAAGACCTTTTGTGATTTCTAGAAGTACTTTTGCAGGGCATGGTAGATATGCTGGACATTGGACAGGGGATGTATGGTCTTCTTGGGAGCAACTAGCTTCATCTGTAC |
| | CTGAGATATTGCAGTTTAATTTGCTAGGAGTGCCACTTGTGGGAGCTGATGTGTGTGGTTTCCTGGGTAATACTAGTGAGGAGCTGTGTGTGAGATGGACACAGCTAGGTGCTTTTTATCCATTTATGAGGAATCATAATTCTCT |
| | GCTGTCACTGCCACAAGAACCATATTCATTTTCTGAACCTGCACAGCAAGCTATGAGAAAGGCATTGACTCTTAGATATGCACTGTTACCTCATCTATATACACTGTTTCATCAGGCTCATGTAGCAGGTGAAACAGTGGCAAGG |
| | CCACTTTTCCTGGAGTTCCCAAAGGATTCTTCTACTTGGACAGTGGATCATCAGCTATTGTGGGGTGAGGCATTATTGATTACTCCTGTGCTTCAAGCTGGAAAAGCAGAGGTGACAGGATATTTCCCTTTAGGAACTTGGTATG |
| | ATCTGCAGACTGTGCCTATAGAGGCACTGGGTTCATTGCCTCCACCACCTGCTGCACCTAGGGAACCTGCAATTCATTCTGAGGGTCAGTGGGTGACATTGCCAGCACCTCTTGATACAATTAATGTGCATTTGAGAGCTGGTTA |
| | TATTATACCTCTGCAGGGTCCTGGGTTGACTACTACTGAATCAAGACAGCAGCCTATGGCTCTGGCAGTGGCACTGACTAAGGGAGGTGAAGCAAGAGGTGAACTTTTTTGGGATGATGGAGAGTCATTAGAAGTACTTGAAAGA |
| | GGGGCATATACTCAGGTGATTTTCTTGGCTAGGAATAATACAATTGTGAATGAACTTGTGAGGGTGACATCAGAGGGAGCAGGTCTGCAGCTTCAGAAGGTGACAGTGCTTGGAGTTGCTACAGCTCCACAGCAAGTTTTAAGTA |
| | ATGGAGTACCTGTGTCTAATTTTACATATAGTCCAGATACAAAAGTGCTGGATATTTGTGTGAGTTTGTTGATGGGTGAGCAATTTCTAGTGAGTTGGTGT |
| 17 | > coGAA GAA nucleic acid |
| | ATGGGTGTTAGGCATCCACCATGTAGTCATAGGCTGCTGGCAGTTTGTGCACTGGTTAGTCTGGCAACTGCAGCACTGCTGGGTCATATTCTGCTGCATGATTTTCTGCTGGTTCCAAGGGAACTGAGTGGTAGTAGTCCAGTTC |
| | TGGAAGAAACTCATCCAGCACATCAACAAGGTGCAAGTAGGCCAGGTCCAAGGGATGCACAAGCACATCCAGGTAGGCCAAGGGCAGTTCCAACTCAATGTGATGTTCCACCAAATAGTAGGTTTGATTGTGCACCAGATAAAGC |
| | AATAACTCAAGAACAATGTGAAGCAAGGGGTTGTTGTTATATTCCAGCAAAACAAGGTCTGCAAGGTGCACAAATGGGTCAACCATGGTGTTTTTTTCCACCAAGTTATCCAAGTTATAAACTGGAAAATCTGAGTAGTAGTGAA |
| | ATGGGTTATACTGCAACTCTGACTAGGACTACTCCAACTTTTTTTCCAAAAGATATTCTGACTCTGAGGCTGGATGTTATGATGGAAACTGAAAATAGGCTGCATTTTACTATTAAAGATCCAGCAAATAGGAGGTATGAAGTTC |
| | CACTGGAAACTCCAAGGGTTCATAGTAGGGCACCAAGTCCACTGTATAGTGTAGAATTTAGTGAAGAACCATTTGGTGTTATTGTTCATAGGCAACTGGATGGTAGGGTTCTGCTGAATACTACTGTTGCACCACTGTTTTTTGC |
| | AGATCAATTTCTGCAACTGAGTACTAGTCTGCCAAGTCAATATATTACTGGTCTGGCAGAACATCTGAGTCCACTGATGCTGAGTACTAGTTGGACTAGGATTACTCTGTGGAATAGGGATCTGGCACCAACTCCAGGTGCAAAT |
| | CTGTATGGTAGTCATCCATTTTATCTGGCACTGGAAGATGGTGGTAGTGCACATGGTGTTTTTCTGCTGAATAGTAATGCAATGGATGTTGTTCTGCAACCAAGTCCAGCACTGAGTTGGAGGAGTACTGGTGGTATTCTGGATG |
| | TTTATATTTTTCTAGGTCCAGAACCAAAAAGTGTTGTTCAACAATATCTGGATGTAGTTGGTTATCCATTTATGCCACCATATTGGGGTCTGGGTTTTCATCTGTGTAGGTGGGGTTATAGTAGTACTGCAATTACTAGGCAAGT |
| | TGTTGAAAATATGACTAGGGCACATTTTCCACTGGATGTTCAATGGAATGATCTAGATTATATGGATAGTAGGAGGGATTTTACTTTTAATAAAGATGGTTTTAGGGATTTTCCAGCAATGGTTCAAGAACTGCATCAAGGTGGT |
| | AGGAGGTATATGATGATTGTTGATCCAGCAATTAGTAGTAGTGGTCCAGCAGGTAGTTATAGGCCATATGATGAAGGTCTGAGGAGGGGTGTTTTTATTACTAATGAAACTGGTCAACCACTGATTGGTAAAGTTTGGCCAGGTA |
| | GTACTGCATTTCCAGATTTTACTAATCCAACTGCACTGGCATGGTGGGAAGATATGGTTGCAGAATTTCATGATCAAGTTCCATTTGATGGTATGTGGATTGATATGAATGAACCAAGTAATTTTATAAGGGGTAGTGAAGATGG |
| | TTGTCCAAATAATGAACTAGAAAATCCACCATATGTTCCAGGTGTTGTTGGTGGTACTCTGCAAGCAGCAACTATTTGTGCAAGTAGTCATCAATTTCTGAGTACTCATTATAATCTGCATAATCTGTATGGTCTGACTGAAGCA |
| | ATTGCAAGTCATAGGGCACTGGTTAAAGCAAGGGGTACTAGGCCATTTGTTATTAGTAGGAGTACTTTTGCAGGTCATGGTAGGTATGCAGGTCATTGGACTGGTGATGTTTGGAGTAGTTGGGAACAACTAGCAAGTAGTGTTC |
| | CAGAAATTCTGCAATTTAATCTGCTGGGTGTTCCACTGGTTGGTGCAGATGTTTGTGGTTTTCTAGGTAATACTAGTGAAGAACTGTGTGTTAGGTGGACTCAACTGGGTGCATTTTATCCATTTATGAGGAATCATAATAGTCT |
| | GCTGAGTCTGCCACAAGAACCATATAGTTTTAGTGAACCAGCACAACAAGCAATGAGGAAAGCACTGACTCTGAGGTATGCACTGCTGCCACATCTGTATACTCTGTTTCATCAAGCACATGTAGCAGGTGAAACTGTTGCAAGG |
| | CCACTGTTTCTGGAATTTCCAAAAGATAGTAGTACTTGGACTGTTGATCATCAACTGCTGTGGGGTGAAGCACTGCTGATTACTCCAGTTCTGCAAGCAGGTAAAGCAGAAGTTACTGGTTATTTTCCACTGGGTACTTGGTATG |
| | ATCTGCAAACTGTTCCAATAGAAGCACTGGGTAGTCTGCCACCACCACCAGCAGCACCAAGGGAACCAGCAATTCATAGTGAAGGTCAATGGGTTACTCTGCCAGCACCACTGGATACTATTAATGTTCATCTGAGGGCAGGTTA |
| | TATTATTCCACTGCAAGGTCCAGGTCTGACTACTACTGAAAGTAGGCAACAACCAATGGCACTGGCAGTTGCACTGACTAAAGGTGGTGAAGCAAGGGGTGAACTGTTTTGGGATGATGGTGAAAGTCTAGAAGTTCTGGAAAGG |
| | GGTGCATATACTCAAGTTATTTTTCTGGCAAGGAATAATACTATTGTTAATGAACTAGTTAGGGTTACTAGTGAAGGTGCAGGTCTGCAACTGCAAAAAGTTACTGTTCTGGGTGTTGCAACTGCACCACAACAAGTTCTGAGTA |
| | ATGGTGTTCCAGTTAGTAATTTTACTTATAGTCCAGATACTAAAGTTCTGGATATTTGTGTTAGTCTGCTGATGGGTGAACAATTTCTGGTTAGTTGGTGT |
| 18 | > coGAA GAA nucleic acid |
| | ATGGGTGTAAGGCATCCTCCATGTAGTCATAGGCTGTTAGCTGTATGTGCACTGGTTAGTCTTGCTACAGCTGCTTTGCTGGGACATATACTACTTCATGATTTTCTGCTGGTGCCAAGAGAGCTAAGTGGGTCTTCACCTGTAT |
| | TGGAGGAGACACATCCAGCACATCAGCAGGGGGCTAGTAGGCCTGGGCCAAGGGATGCACAAGCTCATCCAGGGAGACCTAGAGCAGTTCCAACACAATGTGATGTACCTCCTAATTCAAGGTTTGATTGTGCACCTGATAAAGC |
| | TATAACTCAGGAGCAATGTGAGGCTAGAGGTTGTTGTTATATTCCTGCTAAACAAGGGTTACAAGGGGCTCAAATGGGGCAACCTTGGTGTTTTTTTCCACCAAGTTATCCATCATATAAACTTGAGAATCTGTCTTCAAGTGAA |
| | ATGGGGTATACTGCAACTTTAACAAGGACAACACCAACTTTTTTTCCAAAAGATATTCTGACTCTTAGGCTTGATGTGATGATGGAAACAGAGAATAGGCTGCATTTTACAATTAAAGATCCAGCAAATAGGAGATATGAAGTAC |
| | CTCTAGAAACTCCTAGGGTACATAGTAGAGCACCATCACCACTTTATTCAGTTGAATTTAGTGAAGAACCTTTTGGGGTAATAGTGCATAGACAGTTAGATGGGAGAGTACTTCTGAATACTACTGTTGCTCCTTTGTTTTTTGC |
| | AGATCAGTTTCTTCAACTTTCAACTAGTCTTCCAAGTCAATATATAACTGGTCTTGCTGAGCATTTGAGTCCTCTTATGTTGAGTACATCTTGGACTAGGATTACTCTTTGGAATAGGGATCTGGCACCAACTCCTGGGGCAAAT |
| | TTATATGGATCTCATCCATTTTATTTGGCTTTGGAAGATGGAGGAAGTGCTCATGGAGTATTTTTGTTAAATAGTAATGCTATGGATGTAGTTCTACAGCCTTCTCCTGCTCTGTCATGGAGATCAACAGGTGGGATTTTGGATG |
| | TATATATTTTTTTGGGACCTGAACCTAAGAGTGTTGTGCAACAGTATCTTGATGTTGTAGGGTATCCATTTATGCCACCATATTGGGGGCTTGGATTTCATCTGTGTAGATGGGGATACAGTTCAACTGCTATAACTAGACAGGT |
| | TGTGGAAAATATGACTAGAGCACATTTTCCTTTAGATGTGCAGTGGAATGATCTAGATTACATGGATAGTAGAAGAGATTTTACTTTTAATAAGGATGGGTTTAGAGATTTTCCAGCAATGGTACAGGAACTGCATCAAGGGGGA |
| | AGGAGGTATATGATGATTGTTGACCCTGCAATTAGTAGTTCTGGTCCAGCAGGATCTTATAGACCTTATGATGAGGGGCTTAGGAGGGGTGTATTTATTACAAATGAAACAGGGCAACCATTAATTGGTAAGGTGTGGCCTGGTA |
| | GTACAGCTTTTCCTGATTTTACAAATCCAACAGCACTGGCTTGGTGGGAAGATATGGTGGCAGAATTTCATGATCAAGTGCCTTTTGATGGGATGTGGATTGATATGAATGAGCCAAGTAATTTTATAAGGGGATCTGAGGATGG |
| | ATGTCCAAATAATGAGCTTGAGAATCCTCCTTATGTTCCAGGAGTTGTAGGAGGGACACTGCAAGCAGCAACAATTTGTGCTAGTTCACATCAATTTCTGAGTACACATTATAATTTGCATAATCTGTATGGTTTAACAGAGGCA |
| | ATTGCAAGTCATAGGGCATTAGTGAAAGCAAGAGGTACTAGGCCATTTGTGATTTCTAGGAGTACTTTTGCAGGGCATGGTAGATATGCTGGGCATTGGACAGGAGATGTTTGGAGTAGTTGGGAACAACTAGCATCTTCTGTAC |
| | CTGAGATTCTACAATTTAATCTTCTGGGAGTACCTTTAGTGGGTGCAGATGTATGTGGTTTTCTGGGGAATACTAGTGAGGAATTGTGTGTAAGGTGGACTCAATTAGGAGCATTTTATCCTTTTATGAGGAATCATAATAGTTT |
| | ATTAAGTCTTCCTCAAGAGCCATATTCTTTTTCAGAACCTGCACAGCAAGCAATGAGAAAAGCTCTTACTCTGAGGTATGCATTGCTGCCACATCTGTATACTTTATTTCATCAAGCTCATGTGGCTGGGGAAACTGTAGCAAGA |
| | CCTCTGTTTTTAGAGTTTCCAAAGGATTCTAGTACTTGGACAGTGGATCATCAACTACTTTGGGGTGAGGCTCTGCTTATAACTCCTGTTTTGCAGGCAGGGAAGGCAGAGGTGACTGGTTATTTTCCACTGGGAACATGGTATG |
| | ACCTTCAAACAGTGCCAATAGAAGCACTTGGAAGTCTTCCACCTCCTCCAGCTGCTCCTAGGGAGCCTGCTATACATAGTGAAGGACAATGGGTTACATTACCTGCACCATTAGATACTATTAATGTGCATCTGAGAGCAGGGTA |
| | TATTATTCCTCTGCAAGGGCCAGGACTGACTACTACTGAAAGTAGGCAACAACCAATGGCACTTGCAGTTGCTCTGACAAAAGGTGGTGAAGCAAGAGGTGAGCTTTTTTGGGATGATGGAGAATCATTGGAGGTTCTGGAAAGA |
| | GGGGCATATACTCAGGTTATATTCCTAGCTAGGAACAATACAATAGTTAATGAGTTAGTGAGGGTGACAAGTGAGGGTGCTGGACTTCAATTACAAAAAGTTACTGTTCTGGGTGTAGCAACAGCACCTCAACAGGTTTTGAGTA |
| | ATGGGGTGCCTGTAAGTAATTTTACATACAGTCCAGATACTAAGGTGCTTGATATTTGTGTTAGTTTGCTGATGGGAGAACAATTTTTGGTAAGTTGGTGC |
| 19 | > coGAA GAA nucleic acid w/o signal peptide |
| | GGCCATATCCTGCTGCATGACTTCCTGCTGGTGCCTAGGGAGCTGTCTGGCAGCAGCCCTGTGCTGGAAGAGACCCACCCTGCTCACCAGCAGGGGGCC |
| | AGCAGGCCAGGCCCCAGGGATGCCCAGGCCCACCCTGGCAGACCCAGGGCTGTGCCCACCCAGTGTGATGTGCCTCCCAACAGCAGATTTGACTGTGCCCCTGATAAGGCCATTACTCAG |
| | GAACAGTGTGAGGCCAGGGGCTGCTGCTATATCCCTGCCAAGCAGGGCCTGCAGGGGGCCCAGATGGGGCAGCCCTGGTGCTTCTTCCCCCCCAGCTACCCCTCTTATAAGCTGGAGAAT |
| | CTGAGCAGCTCTGAAATGGGCTACACTGCCACTCTGACCAGGACTACCCCCACCTTCTTCCCCAAGGATATTCTGACTCTGAGGCTGGATGTGATGATGGAGACTGAGAACAGGCTGCAC |
| | TTCACCATCAAGGACCCTGCCAACAGGAGGTATGAGGTGCCCCTGGAAACTCCCAGGGTGCACTCTAGGGCCCCCAGCCCCCTGTATTCTGTGGAGTTCTCTGAGGAGCCTTTTGGGGTC |
| | ATTGTCCACAGGCAGCTGGATGGCAGGGTGCTGCTGAATACTACTGTGGCCCCTCTGTTCTTTGCTGACCAGTTCCTGCAGCTGAGCACCAGCCTGCCTTCTCAGTACATTACTGGCCTG |
| | GCTGAGCATCTGAGCCCCCTGATGCTGAGCACCTCTTGGACCAGAATCACCCTGTGGAACAGGGACCTGGCTCCCACTCCTGGGGCCAACCTGTATGGCAGCCACCCCTTCTACCTGGCC |
| | CTGGAGGATGGGGGCTCTGCCCATGGGGTGTTCCTGCTGAACAGCAATGCCATGGATGTGGTGCTGCAGCCCTCTCCTGCCCTGTCTTGGAGATCTACTGGGGGCATCCTGGATGTGTAT |
| | ATCTTCCTGGGGCCTGAGCCCAAGTCTGTGGTGCAGCAGTACCTGGATGTGGTGGGCTACCCCTTCATGCCCCCCTACTGGGGCCTGGGCTTCCACCTGTGCAGGTGGGGCTACTCTTCT |
| | ACTGCTATCACCAGGCAGGTGGTGGAGAACATGACCAGGGCTCACTTCCCTCTGGATGTGCAGTGGAATGACCTGGACTACATGGACTCTAGGAGAGACTTCACTTTTAATAAGGATGGG |
| | TTCAGGGACTTTCCTGCCATGGTGCAGGAGCTGCATCAGGGGGGCAGGAGATATATGATGATTGTGGACCCTGCTATTTCTAGCTCTGGCCCTGCTGGCAGCTATAGGCCCTATGATGAG |
| | GGGCTGAGGAGGGGGGTGTTCATCACTAATGAGACTGGCCAGCCCCTGATTGGCAAGGTGTGGCCTGGCTCTACTGCCTTCCCTGATTTCACCAACCCCACTGCCCTGGCCTGGTGGGAG |
| | GATATGGTGGCTGAGTTTCATGACCAGGTGCCCTTTGATGGCATGTGGATTGACATGAATGAGCCCAGCAACTTTATCAGGGGCTCTGAAGATGGCTGCCCCAACAATGAGCTGGAGAAC |
| | CCCCCCTATGTGCCTGGGGTGGTGGGGGGCACCCTGCAGGCTGCCACCATCTGTGCCAGCAGCCACCAGTTCCTGAGCACCCACTATAACCTGCACAACCTGTATGGCCTGACTGAGGCC |
| | ATTGCCTCTCACAGGGCCCTGGTGAAGGCTAGGGGGACTAGGCCCTTTGTGATCAGCAGGTCTACTTTTGCTGGCCATGGCAGGTATGCTGGGCACTGGACTGGGGATGTGTGGTCTAGC |
| | TGGGAGCAGCTGGCCAGCTCTGTGCCTGAGATCCTGCAGTTTAATCTGCTGGGGGTGCCCCTGGTGGGGGCTGATGTGTGTGGCTTCCTGGGCAATACCTCTGAGGAGCTGTGTGTGAGG |
| | TGGACTCAGCTGGGGGCTTTCTACCCCTTCATGAGAAACCACAACTCTCTGCTGAGCCTGCCCCAGGAGCCCTATTCTTTTTCTGAGCCTGCCCAGCAGGCTATGAGGAAGGCCCTGACT |
| | CTGAGGTATGCCCTGCTGCCCCACCTGTATACCCTGTTCCATCAGGCCCATGTGGCTGGGGAGACTGTGGCCAGACCTCTGTTCCTGGAGTTCCCCAAGGATAGCTCTACTTGGACTGTG |
| | GACCACCAGCTGCTGTGGGGGGAGGCTCTGCTGATCACCCCTGTGCTGCAGGCTGGGAAGGCTGAGGTGACTGGCTATTTCCCCCTGGGCACCTGGTATGATCTGCAGACTGTGCCCATT |
| | GAGGCCCTGGGCTCTCTGCCCCCTCCCCCTGCTGCCCCCAGGGAGCCTGCCATCCACTCTGAGGGCCAGTGGGTGACCCTGCCTGCCCCTCTGGACACTATCAATGTGCACCTGAGGGCT |
| | GGCTACATCATCCCCCTGCAGGGCCCTGGCCTGACTACCACTGAGTCTAGGCAGCAGCCCATGGCCCTGGCTGTGGCTCTGACCAAGGGGGGGGAGGCCAGGGGGGAGCTGTTCTGGGAT |
| | GATGGGGAGTCTCTGGAGGTGCTGGAGAGGGGGGCCTACACCCAGGTGATCTTCCTGGCTAGGAATAACACCATTGTCAATGAGCTGGTGAGGGTGACCTCTGAGGGGGCTGGCCTGCAG |
| | CTGCAGAAGGTGACTGTGCTGGGGGTGGCTACTGCCCCCCAGCAGGTGCTGAGCAATGGGGTGCCTGTGAGCAACTTCACCTATAGCCCTGACACCAAGGTGCTGGACATCTGTGTGAGC |
| | CTGCTGATGGGGGAGCAGTTCCTGGTGAGCTGGTGCTAA |
| 20 | > coGAA GAA nucleic acid w/o signal peptide |
| | GGACATATTCTGCTGCATGATTTTCTGCTAGTGCCTAGAGAGCTGTCTGGATCTTCTCCTGTGCTGGAGGAGACACATCCTGCTCATCAGCAGGGAGCT |
| | TCTAGACCTGGACCTAGAGATGCTCAGGCTCATCCTGGAAGACCTAGAGCTGTGCCTACACAGTGTGATGTGCCTCCTAATTCTAGATTTGATTGTGCTCCTGATAAGGCTATAACACAG |
| | GAGCAGTGTGAGGCTAGAGGATGTTGTTATATTCCTGCTAAGCAGGGACTGCAGGGAGCTCAGATGGGACAGCCTTGGTGTTTTTTTCCTCCTTCTTATCCTTCTTATAAGCTGGAGAAT |
| | CTGTCTTCTTCTGAGATGGGATATACAGCTACACTGACAAGAACAACACCTACATTTTTTCCTAAGGATATTCTGACACTGAGACTGGATGTGATGATGGAGACAGAGAATAGACTGCAT |
| | TTTACAATTAAGGATCCTGCTAATAGAAGATATGAGGTGCCTCTAGAGACACCTAGAGTGCATTCTAGAGCTCCTTCTCCTCTGTATTCTGTGGAGTTTTCTGAGGAGCCTTTTGGAGTG |
| | ATTGTGCATAGACAGCTGGATGGAAGAGTGCTGCTGAATACAACAGTGGCTCCTCTGTTTTTTGCTGATCAGTTTCTGCAGCTGTCTACATCTCTGCCTTCTCAGTATATTACAGGACTG |
| | GCTGAGCATCTGTCTCCTCTGATGCTGTCTACATCTTGGACAAGAATTACACTGTGGAATAGAGATCTGGCTCCTACACCTGGAGCTAATCTGTATGGATCTCATCCTTTTTATCTGGCT |
| | CTGGAGGATGGAGGATCTGCTCATGGAGTGTTTCTGCTGAATTCTAATGCTATGGATGTAGTGCTGCAGCCTTCTCCTGCTCTGTCTTGGAGATCTACAGGAGGAATTCTGGATGTGTAT |
| | ATTTTTCTAGGACCTGAGCCTAAGTCTGTGGTGCAGCAGTATCTGGATGTAGTGGGATATCCTTTTATGCCTCCTTATTGGGGACTGGGATTTCATCTGTGTAGATGGGGATATTCTTCT |
| | ACAGCTATTACAAGACAGGTGGTTGAGAATATGACAAGAGCTCATTTTCCTCTGGATGTGCAGTGGAATGATCTAGATTATATGGATTCTAGAAGAGATTTTACATTTAATAAGGATGGA |
| | TTTAGAGATTTTCCTGCTATGGTGCAGGAGCTGCATCAGGGAGGAAGAAGATATATGATGATTGTGGATCCTGCTATTTCTTCTTCTGGACCTGCTGGATCTTATAGACCTTATGATGAG |
| | GGACTGAGAAGAGGAGTGTTTATTACAAATGAGACAGGACAGCCTCTGATTGGAAAGGTGTGGCCTGGATCTACAGCTTTTCCTGATTTTACAAATCCTACAGCTCTGGCTTGGTGGGAG |
| | GATATGGTGGCTGAGTTTCATGATCAGGTGCCTTTTGATGGAATGTGGATTGATATGAATGAGCCTTCTAATTTTATAAGAGGATCTGAGGATGGATGTCCTAATAATGAGCTAGAGAAT |
| | CCTCCTTATGTGCCTGGAGTAGTGGGAGGAACACTGCAGGCTGCTACAATTTGTGCTTCTTCTCATCAGTTTCTGTCTACACATTATAATCTGCATAATCTGTATGGACTGACAGAGGCT |
| | ATTGCTTCTCATAGAGCTCTGGTGAAGGCTAGAGGAACAAGACCTTTTGTGATTTCTAGATCTACATTTGCTGGACATGGAAGATATGCTGGACATTGGACAGGAGATGTATGGTCTTCT |
| | TGGGAGCAGCTAGCTTCTTCTGTGCCTGAGATTCTGCAGTTTAATCTGCTAGGAGTGCCTCTGGTGGGAGCTGATGTGTGTGGATTTCTGGGAAATACATCTGAGGAGCTGTGTGTGAGA |
| | TGGACACAGCTGGGAGCTTTTTATCCTTTTATGAGAAATCATAATTCTCTGCTGTCTCTGCCTCAGGAGCCTTATTCTTTTTCTGAGCCTGCTCAGCAGGCTATGAGAAAGGCTCTGACA |
| | CTGAGATATGCTCTGCTGCCTCATCTGTATACACTGTTTCATCAGGCTCATGTGGCTGGAGAGACAGTAGCTAGACCTCTGTTTCTGGAGTTTCCTAAGGATTCTTCTACATGGACAGTT |
| | GATCATCAGCTGCTGTGGGGAGAGGCTCTGCTGATTACACCTGTGCTGCAGGCTGGAAAGGCTGAGGTGACAGGATATTTTCCTCTGGGAACATGGTATGATCTGCAGACAGTGCCTATA |
| | GAGGCTCTGGGATCTCTGCCTCCTCCTCCTGCTGCTCCTAGAGAGCCTGCTATTCATTCTGAGGGACAGTGGGTGACACTGCCTGCTCCTCTGGATACAATTAATGTGCATCTGAGAGCT |
| | GGATATATTATTCCTCTGCAGGGACCTGGACTGACAACAACAGAGTCTAGACAGCAGCCTATGGCTCTGGCTGTGGCTCTGACAAAGGGAGGAGAGGCTAGAGGAGAGCTGTTTTGGGAT |
| | GATGGAGAGTCTCTGGAGGTGCTGGAGAGAGGAGCTTATACACAGGTGATTTTTCTGGCTAGAAATAATACAATTGTGAATGAGCTGGTGAGAGTGACATCTGAGGGAGCTGGACTGCAG |
| | CTGCAGAAGGTGACAGTGCTGGGAGTGGCTACAGCTCCTCAGCAGGTGCTGTCTAATGGAGTGCCTGTGTCTAATTTTACATATTCTCCTGATACAAAGGTGCTGGATATTTGTGTGTCT |
| | CTGCTGATGGGAGAGCAGTTTCTGGTGTCTTGGTGT |
| 21 | > coGAA GAA nucleic acid |
| | CAGCAGGGG GCCAGCAGGC CAGGCCCCAG GGATGCCCAG GCCCACCCTG GCAGACCCAG GGCTGTGCCC ACCCAGTGTG ATGTGCCTCC CAACAGCAGA TTTGACTGTG CCCCTGATAA GGCCATTACT |
| | CAGGAACAGT GTGAGGCCAG GGGCTGCTGC TATATCCCTG CCAAGCAGGG CCTGCAGGGG GCCCAGATGG GGCAGCCCTG GTGCTTCTTC CCCCCCAGCT ACCCCTCTTA TAAGCTGGAG |
| | AATCTGAGCA GCTCTGAAAT GGGCTACACT GCCACTCTGA CCAGGACTAC CCCCACCTTC TTCCCCAAGG ATATTCTGAC TCTGAGGCTG GATGTGATGA TGGAGACTGA GAACAGGCTG |
| | CACTTCACCA TCAAGGACCC TGCCAACAGG AGGTATGAGG TGCCCCTGGA AACTCCCAGG GTGCACTCTA GGGCCCCCAG CCCCCTGTAT TCTGTGGAGT TCTCTGAGGA GCCTTTTGGG |
| | GTCATTGTCC ACAGGCAGCT GGATGGCAGG GTGCTGCTGA ATACTACTGT GGCCCCTCTG TTCTTTGCTG ACCAGTTCCT GCAGCTGAGC ACCAGCCTGC CTTCTCAGTA CATTACTGGC |
| | CTGGCTGAGC ATCTGAGCCC CCTGATGCTG AGCACCTCTT GGACCAGAAT CACCCTGTGG AACAGGGACC TGGCTCCCAC TCCTGGGGCC AACCTGTATG GCAGCCACCC CTTCTACCTG |
| | GCCCTGGAGG ATGGGGGCTC TGCCCATGGG GTGTTCCTGC TGAACAGCAA TGCCATGGAT GTGGTGCTGC AGCCCTCTCC TGCCCTGTCT TGGAGATCTA CTGGGGGCAT CCTGGATGTG |
| | TATATCTTCC TGGGGCCTGA GCCCAAGTCT GTGGTGCAGC AGTACCTGGA TGTGGTGGGC TACCCCTTCA TGCCCCCCTA CTGGGGCCTG GGCTTCCACC TGTGCAGGTG GGGCTACTCT |
| | TCTACTGCTA TCACCAGGCA GGTGGTGGAG AACATGACCA GGGCTCACTT CCCTCTGGAT GTGCAGTGGA ATGACCTGGA CTACATGGAC TCTAGGAGAG ACTTCACTTT TAATAAGGAT |
| | GGGTTCAGGG ACTTTCCTGC CATGGTGCAG GAGCTGCATC AGGGGGGCAG GAGATATATG ATGATTGTGG ACCCTGCTAT TTCTAGCTCT GGCCCTGCTG GCAGCTATAG GCCCTATGAT |
| | GAGGGGCTGA GGAGGGGGGT GTTCATCACT AATGAGACTG GCCAGCCCCT GATTGGCAAG GTGTGGCCTG GCTCTACTGC CTTCCCTGAT TTCACCAACC CCACTGCCCT GGCCTGGTGG |
| | GAGGATATGG TGGCTGAGTT TCATGACCAG GTGCCCTTTG ATGGCATGTG GATTGACATG AATGAGCCCA GCAACTTTAT CAGGGGCTCT GAAGATGGCT GCCCCAACAA TGAGCTGGAG |
| | AACCCCCCCT ATGTGCCTGG GGTGGTGGGG GGCACCCTGC AGGCTGCCAC CATCTGTGCC AGCAGCCACC AGTTCCTGAG CACCCACTAT AACCTGCACA ACCTGTATGG CCTGACTGAG |
| | GCCATTGCCT CTCACAGGGC CCTGGTGAAG GCTAGGGGGA CTAGGCCCTT TGTGATCAGC AGGTCTACTT TTGCTGGCCA TGGCAGGTAT GCTGGGCACT GGACTGGGGA TGTGTGGTCT |
| | AGCTGGGAGC AGCTGGCCAG CTCTGTGCCT GAGATCCTGC AGTTTAATCT GCTGGGGGTG CCCCTGGTGG GGGCTGATGT GTGTGGCTTC CTGGGCAATA CCTCTGAGGA GCTGTGTGTG |
| | AGGTGGACTC AGCTGGGGGC TTTCTACCCC TTCATGAGAA ACCACAACTC TCTGCTGAGC CTGCCCCAGG AGCCCTATTC TTTTTCTGAG CCTGCCCAGC AGGCTATGAG GAAGGCCCTG |
| | ACTCTGAGGT ATGCCCTGCT GCCCCACCTG TATACCCTGT TCCATCAGGC CCATGTGGCT GGGGAGACTG TGGCCAGACC TCTGTTCCTG GAGTTCCCCA AGGATAGCTC TACTTGGACT |
| | GTGGACCACC AGCTGCTGTG GGGGGAGGCT CTGCTGATCA CCCCTGTGCT GCAGGCTGGG AAGGCTGAGG TGACTGGCTA TTTCCCCCTG GGCACCTGGT ATGATCTGCA GACTGTGCCC |
| | ATTGAGGCCC TGGGCTCTCT GCCCCCTCCC CCTGCTGCCC CCAGGGAGCC TGCCATCCAC TCTGAGGGCC AGTGGGTGAC CCTGCCTGCC CCTCTGGACA CTATCAATGT GCACCTGAGG |
| | GCTGGCTACA TCATCCCCCT GCAGGGCCCT GGCCTGACTA CCACTGAGTC TAGGCAGCAG CCCATGGCCC TGGCTGTGGC TCTGACCAAG GGGGGGGAGG CCAGGGGGGA GCTGTTCTGG |
| | GATGATGGGG AGTCTCTGGA GGTGCTGGAG AGGGGGGCCT ACACCCAGGT GATCTTCCTG GCTAGGAATA ACACCATTGT CAATGAGCTG GTGAGGGTGA CCTCTGAGGG GGCTGGCCTG |
| | CAGCTGCAGA AGGTGACTGT GCTGGGGGTG GCTACTGCCC CCCAGCAGGT GCTGAGCAAT GGGGTGCCTG TGAGCAACTT CACCTATAGC CCTGACACCA AGGTGCTGGA CATCTGTGTG |
| | AGCCTGCTGA TGGGGGAGCA GTTCCTGGTG AGCTGGTGCT AA |
| 22 | > coGAA GAA nucleic acid w/ hFN1 signal peptide |
| | ATGCTTAGGGGTCCGGGGCCCGGGCTGCTGCTGCTGGCCGTCCAGTGCCTGGGGACAGCGGTGCCCTCCACGGGAGCCTCGAAGAGCAAGAGG |
| | GGCCATATCCTGCTGCATGACTTCCTGCTGGTGCCTAGGGAGCTGTCTGGCAGCAGCCCTGTGCTGGAAGAGACCCACCCTGCTCACCAGCAGGGGGCC |
| | AGCAGGCCAGGCCCCAGGGATGCCCAGGCCCACCCTGGCAGACCCAGGGCTGTGCCCACCCAGTGTGATGTGCCTCCCAACAGCAGATTTGACTGTGCCCCTGATAAGGCCATTACTCAG |
| | GAACAGTGTGAGGCCAGGGGCTGCTGCTATATCCCTGCCAAGCAGGGCCTGCAGGGGGCCCAGATGGGGCAGCCCTGGTGCTTCTTCCCCCCCAGCTACCCCTCTTATAAGCTGGAGAAT |
| | CTGAGCAGCTCTGAAATGGGCTACACTGCCACTCTGACCAGGACTACCCCCACCTTCTTCCCCAAGGATATTCTGACTCTGAGGCTGGATGTGATGATGGAGACTGAGAACAGGCTGCAC |
| | TTCACCATCAAGGACCCTGCCAACAGGAGGTATGAGGTGCCCCTGGAAACTCCCAGGGTGCACTCTAGGGCCCCCAGCCCCCTGTATTCTGTGGAGTTCTCTGAGGAGCCTTTTGGGGTC |
| | ATTGTCCACAGGCAGCTGGATGGCAGGGTGCTGCTGAATACTACTGTGGCCCCTCTGTTCTTTGCTGACCAGTTCCTGCAGCTGAGCACCAGCCTGCCTTCTCAGTACATTACTGGCCTG |
| | GCTGAGCATCTGAGCCCCCTGATGCTGAGCACCTCTTGGACCAGAATCACCCTGTGGAACAGGGACCTGGCTCCCACTCCTGGGGCCAACCTGTATGGCAGCCACCCCTTCTACCTGGCC |
| | CTGGAGGATGGGGGCTCTGCCCATGGGGTGTTCCTGCTGAACAGCAATGCCATGGATGTGGTGCTGCAGCCCTCTCCTGCCCTGTCTTGGAGATCTACTGGGGGCATCCTGGATGTGTAT |
| | ATCTTCCTGGGGCCTGAGCCCAAGTCTGTGGTGCAGCAGTACCTGGATGTGGTGGGCTACCCCTTCATGCCCCCCTACTGGGGCCTGGGCTTCCACCTGTGCAGGTGGGGCTACTCTTCT |
| | ACTGCTATCACCAGGCAGGTGGTGGAGAACATGACCAGGGCTCACTTCCCTCTGGATGTGCAGTGGAATGACCTGGACTACATGGACTCTAGGAGAGACTTCACTTTTAATAAGGATGGG |
| | TTCAGGGACTTTCCTGCCATGGTGCAGGAGCTGCATCAGGGGGGCAGGAGATATATGATGATTGTGGACCCTGCTATTTCTAGCTCTGGCCCTGCTGGCAGCTATAGGCCCTATGATGAG |
| | GGGCTGAGGAGGGGGGTGTTCATCACTAATGAGACTGGCCAGCCCCTGATTGGCAAGGTGTGGCCTGGCTCTACTGCCTTCCCTGATTTCACCAACCCCACTGCCCTGGCCTGGTGGGAG |
| | GATATGGTGGCTGAGTTTCATGACCAGGTGCCCTTTGATGGCATGTGGATTGACATGAATGAGCCCAGCAACTTTATCAGGGGCTCTGAAGATGGCTGCCCCAACAATGAGCTGGAGAAC |
| | CCCCCCTATGTGCCTGGGGTGGTGGGGGGCACCCTGCAGGCTGCCACCATCTGTGCCAGCAGCCACCAGTTCCTGAGCACCCACTATAACCTGCACAACCTGTATGGCCTGACTGAGGCC |
| | ATTGCCTCTCACAGGGCCCTGGTGAAGGCTAGGGGGACTAGGCCCTTTGTGATCAGCAGGTCTACTTTTGCTGGCCATGGCAGGTATGCTGGGCACTGGACTGGGGATGTGTGGTCTAGC |
| | TGGGAGCAGCTGGCCAGCTCTGTGCCTGAGATCCTGCAGTTTAATCTGCTGGGGGTGCCCCTGGTGGGGGCTGATGTGTGTGGCTTCCTGGGCAATACCTCTGAGGAGCTGTGTGTGAGG |
| | TGGACTCAGCTGGGGGCTTTCTACCCCTTCATGAGAAACCACAACTCTCTGCTGAGCCTGCCCCAGGAGCCCTATTCTTTTTCTGAGCCTGCCCAGCAGGCTATGAGGAAGGCCCTGACT |
| | CTGAGGTATGCCCTGCTGCCCCACCTGTATACCCTGTTCCATCAGGCCCATGTGGCTGGGGAGACTGTGGCCAGACCTCTGTTCCTGGAGTTCCCCAAGGATAGCTCTACTTGGACTGTG |
| | GACCACCAGCTGCTGTGGGGGGAGGCTCTGCTGATCACCCCTGTGCTGCAGGCTGGGAAGGCTGAGGTGACTGGCTATTTCCCCCTGGGCACCTGGTATGATCTGCAGACTGTGCCCATT |
| | GAGGCCCTGGGCTCTCTGCCCCCTCCCCCTGCTGCCCCCAGGGAGCCTGCCATCCACTCTGAGGGCCAGTGGGTGACCCTGCCTGCCCCTCTGGACACTATCAATGTGCACCTGAGGGCT |
| | GGCTACATCATCCCCCTGCAGGGCCCTGGCCTGACTACCACTGAGTCTAGGCAGCAGCCCATGGCCCTGGCTGTGGCTCTGACCAAGGGGGGGGAGGCCAGGGGGGAGCTGTTCTGGGAT |
| | GATGGGGAGTCTCTGGAGGTGCTGGAGAGGGGGGCCTACACCCAGGTGATCTTCCTGGCTAGGAATAACACCATTGTCAATGAGCTGGTGAGGGTGACCTCTGAGGGGGCTGGCCTGCAG |
| | CTGCAGAAGGTGACTGTGCTGGGGGTGGCTACTGCCCCCCAGCAGGTGCTGAGCAATGGGGTGCCTGTGAGCAACTTCACCTATAGCCCTGACACCAAGGTGCTGGACATCTGTGTGAGC |
| | CTGCTGATGGGGGAGCAGTTCCTGGTGAGCTGGTGCTAA |
| 23 | > ITR-to-ITR construct with coGAA nucleic acid |
| | >[L-ITR] - [ACTUS101-LSP] - [5′UTR] - [coGAA (w/sp)] - [3′UTR] - [PolIII_terminator] - [hGHpolyA] - [PolII_terminator-rev][R-ITR] |
| | CTGCGCGCTCGCTCGCTCACTGAGGCCGCCCGGGCAAAGCCCGGGCGTCGGGCGACCTTTGGTCGCCCGGCCTCAGTGAGCGAGCGAGCGCGCAGAGAGGGAGTGGCCAACTCCATCACTAGGGGTTCCTTGTAGTTAATGATTA |
| | ACCCGCCATGCTACTTATCTACGGCGCGCCACGCGTTCTAGGAGTTAATTTTTAAAAAGCAGTCAAAAGTCCAAGTGCCCTTGCGAGCATTTACTCTCTCTGTTTGCTCTGGTTAATAATCTCAGGAGCACAAACATTCCTTACT |
| | AGTTCTAGGAGTTAATTTTTAAAAAGCAGTCAAAAGTCCAAGTGCCCTTGCGAGCATTTACTCTCTCTGTTTGCTCTGGTTAATAATCTCAGGAGCACAAACATTCCTTACTAGTTCTAGAGCGGCCGCCAGTGTGCTGGAATTC |
| | GGCTTTTTTAGGGCTGGAAGCTACCTTTGACATCATTTCCTCTGCGAATGCATGTATAATTTCTACAGAACCTATTAGAAAGGATCACCCAGCCTCTGCTTTTGTACAACTTTCCCTTAAAAAACTGCCAATCCCACTGCTGTTT |
| | GGCCCAATAGTGAGAACTTTTTCCTGCTGCCTCTTGGTGCTTTTGCCTATGGCCCCTATTCTGCCTGCTGAAGACACTCTTGCCAGCATGGACTTAAACCCCTCCAGCTCTGACAATCCTCTTTCTCTTTTGTTTTACATGAAGG |
| | GTCTGGCAGCCAAAGCAATCACTCAAAGTTCAAACCTTATCATTTTTTGCTTTGTTCCTCTTGGCCTTGGTTTTGTACATCAGCTTTGAAAATACCATCCCAGGGTTAATGCTGGGGTTAATTTATAACTGAGAGTGCTCTAGTT |
| | CTGCAATACAGGACATGCTATAAAAATGGAAAGATGTTGCTTTCTGAGAGATCAGGTGGATTCTTGGGCATTTGCTTAAGCTTGGTACCGCCACCATGGGGGTGAGGCACCCTCCCTGTTCTCACAGGCTGCTGGCTGTGTGTGC |
| | TCTGGTGTCTCTGGCCACTGCTGCCCTGCTGGGCCATATCCTGCTGCATGACTTCCTGCTGGTGCCTAGGGAGCTGTCTGGCAGCAGCCCTGTGCTGGAAGAGACCCACCCTGCTCACCAGCAGGGGGCCAGCAGGCCAGGCCCC |
| | AGGGATGCCCAGGCCCACCCTGGCAGACCCAGGGCTGTGCCCACCCAGTGTGATGTGCCTCCCAACAGCAGATTTGACTGTGCCCCTGATAAGGCCATTACTCAGGAACAGTGTGAGGCCAGGGGCTGCTGCTATATCCCTGCCA |
| | AGCAGGGCCTGCAGGGGGCCCAGATGGGGCAGCCCTGGTGCTTCTTCCCCCCCAGCTACCCCTCTTATAAGCTGGAGAATCTGAGCAGCTCTGAAATGGGCTACACTGCCACTCTGACCAGGACTACCCCCACCTTCTTCCCCAA |
| | GGATATTCTGACTCTGAGGCTGGATGTGATGATGGAGACTGAGAACAGGCTGCACTTCACCATCAAGGACCCTGCCAACAGGAGGTATGAGGTGCCCCTGGAAACTCCCAGGGTGCACTCTAGGGCCCCCAGCCCCCTGTATTCT |
| | GTGGAGTTCTCTGAGGAGCCTTTTGGGGTCATTGTCCACAGGCAGCTGGATGGCAGGGTGCTGCTGAATACTACTGTGGCCCCTCTGTTCTTTGCTGACCAGTTCCTGCAGCTGAGCACCAGCCTGCCTTCTCAGTACATTACTG |
| | GCCTGGCTGAGCATCTGAGCCCCCTGATGCTGAGCACCTCTTGGACCAGAATCACCCTGTGGAACAGGGACCTGGCTCCCACTCCTGGGGCCAACCTGTATGGCAGCCACCCCTTCTACCTGGCCCTGGAGGATGGGGGCTCTGC |
| | CCATGGGGTGTTCCTGCTGAACAGCAATGCCATGGATGTGGTGCTGCAGCCCTCTCCTGCCCTGTCTTGGAGATCTACTGGGGGCATCCTGGATGTGTATATCTTCCTGGGGCCTGAGCCCAAGTCTGTGGTGCAGCAGTACCTG |
| | GATGTGGTGGGCTACCCCTTCATGCCCCCCTACTGGGGCCTGGGCTTCCACCTGTGCAGGTGGGGCTACTCTTCTACTGCTATCACCAGGCAGGTGGTGGAGAACATGACCAGGGCTCACTTCCCTCTGGATGTGCAGTGGAATG |
| | ACCTGGACTACATGGACTCTAGGAGAGACTTCACTTTTAATAAGGATGGGTTCAGGGACTTTCCTGCCATGGTGCAGGAGCTGCATCAGGGGGGCAGGAGATATATGATGATTGTGGACCCTGCTATTTCTAGCTCTGGCCCTGC |
| | TGGCAGCTATAGGCCCTATGATGAGGGGCTGAGGAGGGGGGTGTTCATCACTAATGAGACTGGCCAGCCCCTGATTGGCAAGGTGTGGCCTGGCTCTACTGCCTTCCCTGATTTCACCAACCCCACTGCCCTGGCCTGGTGGGAG |
| | GATATGGTGGCTGAGTTTCATGACCAGGTGCCCTTTGATGGCATGTGGATTGACATGAATGAGCCCAGCAACTTTATCAGGGGCTCTGAAGATGGCTGCCCCAACAATGAGCTGGAGAACCCCCCCTATGTGCCTGGGGTGGTGG |
| | GGGGCACCCTGCAGGCTGCCACCATCTGTGCCAGCAGCCACCAGTTCCTGAGCACCCACTATAACCTGCACAACCTGTATGGCCTGACTGAGGCCATTGCCTCTCACAGGGCCCTGGTGAAGGCTAGGGGGACTAGGCCCTTTGT |
| | GATCAGCAGGTCTACTTTTGCTGGCCATGGCAGGTATGCTGGGCACTGGACTGGGGATGTGTGGTCTAGCTGGGAGCAGCTGGCCAGCTCTGTGCCTGAGATCCTGCAGTTTAATCTGCTGGGGGTGCCCCTGGTGGGGGCTGAT |
| | GTGTGTGGCTTCCTGGGCAATACCTCTGAGGAGCTGTGTGTGAGGTGGACTCAGCTGGGGGCTTTCTACCCCTTCATGAGAAACCACAACTCTCTGCTGAGCCTGCCCCAGGAGCCCTATTCTTTTTCTGAGCCTGCCCAGCAGG |
| | CTATGAGGAAGGCCCTGACTCTGAGGTATGCCCTGCTGCCCCACCTGTATACCCTGTTCCATCAGGCCCATGTGGCTGGGGAGACTGTGGCCAGACCTCTGTTCCTGGAGTTCCCCAAGGATAGCTCTACTTGGACTGTGGACCA |
| | CCAGCTGCTGTGGGGGGAGGCTCTGCTGATCACCCCTGTGCTGCAGGCTGGGAAGGCTGAGGTGACTGGCTATTTCCCCCTGGGCACCTGGTATGATCTGCAGACTGTGCCCATTGAGGCCCTGGGCTCTCTGCCCCCTCCCCCT |
| | GCTGCCCCCAGGGAGCCTGCCATCCACTCTGAGGGCCAGTGGGTGACCCTGCCTGCCCCTCTGGACACTATCAATGTGCACCTGAGGGCTGGCTACATCATCCCCCTGCAGGGCCCTGGCCTGACTACCACTGAGTCTAGGCAGC |
| | AGCCCATGGCCCTGGCTGTGGCTCTGACCAAGGGGGGGGAGGCCAGGGGGGAGCTGTTCTGGGATGATGGGGAGTCTCTGGAGGTGCTGGAGAGGGGGGCCTACACCCAGGTGATCTTCCTGGCTAGGAATAACACCATTGTCAA |
| | TGAGCTGGTGAGGGTGACCTCTGAGGGGGCTGGCCTGCAGCTGCAGAAGGTGACTGTGCTGGGGGTGGCTACTGCCCCCCAGCAGGTGCTGAGCAATGGGGTGCCTGTGAGCAACTTCACCTATAGCCCTGACACCAAGGTGCTG |
| | GACATCTGTGTGAGCCTGCTGATGGGGGAGCAGTTCCTGGTGAGCTGGTGCTAATTTAAATCTCGAGCCGGGCGGAGTGTGTTAGTCTCTCCAGAGGGAGGCTGGTTCCCCAGGGAAGCAGAGCCTGTGTGCGGGCAGCAGCTGT |
| | GTGCGGGCCTGGGGGTTGTTAAGTGCAATTATTTTTAATAAAAGGGGCATTTGGAAAAAAAAAAAAAAGGTAGCAGTCGACAGATGAATTCTGCAGATCTGTGGCTTCTAGCTGCCCGGGTGGCATCCCTGTGACCCCTCCCCAG |
| | TGCCTCTCCTGGCCCTGGAAGTTGCCACTCCAGTGCCCACCAGCCTTGTCCTAATAAAATTAAGTTGCATCATTTTGTCTGACTAGGTGTCCTTCTATAATATTATGGGGTGGAGGGGGGTGGTATGGAGCAAGGGGCAAGTTGG |
| | GAAGACAACCTGTAGGGCCTGCGGGGTCTATTGGGAACCAAGCTGGAGTGCAGTGGCACAATCTTGGCTCACTGCAATCTCCGCCTCCTGGGTTCAAGCGATTCTCCTGCCTCAGCCTCCCGAGTTGTTGGGATTCCAGGCATGC |
| | ATGACCAGGCTCAGCTAATTTTTGTTTTTTTGGTAGAGACGGGGTTTCACCATATTGGCCAGGCTGGTCTCCAACTCCTAATCTCAGGTGATCTACCCACCTTGGCCTCCCAAATTGCTGGGATTACAGGCGTGAACCACTGCTC |
| | CCTTCCCTGTCCTTCTGATTTTAAAAAAAAGAGAAATGTTCTGGCACCTGCACTTGCACTGGGGACAGCCTATTTTGCTAGTTTGTTTTGTTTCGTTTTGTTTTGATGGAGAGCGTATGTTGTTTAAACGCGGCCGCGTAGATAA |
| | GTAGCATGGCGGGTTAATCATTAACTACAAGGAACCCCTAGTGATGGAGTTGGCCACTCCCTCTCTGCGCGCTCGCTCGCTCACTGAGGCCGGGCGACCAAAGGTCGCCCGACGCCCGGGCTTTGCCCGGGCGGCCTCAGTGAGC |
| | GAGCGAGCGCGCAG |
| 24 | > ITR-to-ITR construct with coGAA GAA nucleic acid |
| | >[L-ITR] - [ACTUS101-LSP] - [5′UTR] - [coGAA w/sp)] - [3′UTR] - [PolIII_terminator] - [hGHpolyA] - [PolII_terminator-rev][R-ITR] |
| | CTGCGCGCTCGCTCGCTCACTGAGGCCGCCCGGGCAAAGCCCGGGCGTCGGGCGACCTTTGGTCGCCCGGCCTCAGTGAGCGAGCGAGCGCGCAGAGAGGGAGTGGCCAACTCCATCACTAGGGGTTCCTTGTAGTTAATGATTA |
| | ACCCGCCATGCTACTTATCTACGGCGCGCCACGCGTTCTAGGAGTTAATTTTTAAAAAGCAGTCAAAAGTCCAAGTGCCCTTGCGAGCATTTACTCTCTCTGTTTGCTCTGGTTAATAATCTCAGGAGCACAAACATTCCTTACT |
| | AGTTCTAGGAGTTAATTTTTAAAAAGCAGTCAAAAGTCCAAGTGCCCTTGCGAGCATTTACTCTCTCTGTTTGCTCTGGTTAATAATCTCAGGAGCACAAACATTCCTTACTAGTTCTAGAGCGGCCGCCAGTGTGCTGGAATTC |
| | GGCTTTTTTAGGGCTGGAAGCTACCTTTGACATCATTTCCTCTGCGAATGCATGTATAATTTCTACAGAACCTATTAGAAAGGATCACCCAGCCTCTGCTTTTGTACAACTTTCCCTTAAAAAACTGCCAATCCCACTGCTGTTT |
| | GGCCCAATAGTGAGAACTTTTTCCTGCTGCCTCTTGGTGCTTTTGCCTATGGCCCCTATTCTGCCTGCTGAAGACACTCTTGCCAGCATGGACTTAAACCCCTCCAGCTCTGACAATCCTCTTTCTCTTTTGTTTTACATGAAGG |
| | GTCTGGCAGCCAAAGCAATCACTCAAAGTTCAAACCTTATCATTTTTTGCTTTGTTCCTCTTGGCCTTGGTTTTGTACATCAGCTTTGAAAATACCATCCCAGGGTTAATGCTGGGGTTAATTTATAACTGAGAGTGCTCTAGTT |
| | CTGCAATACAGGACATGCTATAAAAATGGAAAGATGTTGCTTTCTGAGAGATCAGGTGGATTCTTGGGCATTTGCTTAAGCTTGGTACCGCCACCATGGGAGTGAGACATCCTCCTTGTTCTCATAGACTGCTGGCTGTGTGTGC |
| | TCTGGTGTCTCTGGCTACAGCTGCTCTGCTGGGACATATTCTGCTGCATGATTTTCTGCTAGTGCCTAGAGAGCTGTCTGGATCTTCTCCTGTGCTGGAGGAGACACATCCTGCTCATCAGCAGGGAGCTTCTAGACCTGGACCT |
| | AGAGATGCTCAGGCTCATCCTGGAAGACCTAGAGCTGTGCCTACACAGTGTGATGTGCCTCCTAATTCTAGATTTGATTGTGCTCCTGATAAGGCTATAACACAGGAGCAGTGTGAGGCTAGAGGATGTTGTTATATTCCTGCTA |
| | AGCAGGGACTGCAGGGAGCTCAGATGGGACAGCCTTGGTGTTTTTTTCCTCCTTCTTATCCTTCTTATAAGCTGGAGAATCTGTCTTCTTCTGAGATGGGATATACAGCTACACTGACAAGAACAACACCTACATTTTTTCCTAA |
| | GGATATTCTGACACTGAGACTGGATGTGATGATGGAGACAGAGAATAGACTGCATTTTACAATTAAGGATCCTGCTAATAGAAGATATGAGGTGCCTCTAGAGACACCTAGAGTGCATTCTAGAGCTCCTTCTCCTCTGTATTCT |
| | GTGGAGTTTTCTGAGGAGCCTTTTGGAGTGATTGTGCATAGACAGCTGGATGGAAGAGTGCTGCTGAATACAACAGTGGCTCCTCTGTTTTTTGCTGATCAGTTTCTGCAGCTGTCTACATCTCTGCCTTCTCAGTATATTACAG |
| | GACTGGCTGAGCATCTGTCTCCTCTGATGCTGTCTACATCTTGGACAAGAATTACACTGTGGAATAGAGATCTGGCTCCTACACCTGGAGCTAATCTGTATGGATCTCATCCTTTTTATCTGGCTCTGGAGGATGGAGGATCTGC |
| | TCATGGAGTGTTTCTGCTGAATTCTAATGCTATGGATGTAGTGCTGCAGCCTTCTCCTGCTCTGTCTTGGAGATCTACAGGAGGAATTCTGGATGTGTATATTTTTCTAGGACCTGAGCCTAAGTCTGTGGTGCAGCAGTATCTG |
| | GATGTAGTGGGATATCCTTTTATGCCTCCTTATTGGGGACTGGGATTTCATCTGTGTAGATGGGGATATTCTTCTACAGCTATTACAAGACAGGTGGTTGAGAATATGACAAGAGCTCATTTTCCTCTGGATGTGCAGTGGAATG |
| | ATCTAGATTATATGGATTCTAGAAGAGATTTTACATTTAATAAGGATGGATTTAGAGATTTTCCTGCTATGGTGCAGGAGCTGCATCAGGGAGGAAGAAGATATATGATGATTGTGGATCCTGCTATTTCTTCTTCTGGACCTGC |
| | TGGATCTTATAGACCTTATGATGAGGGACTGAGAAGAGGAGTGTTTATTACAAATGAGACAGGACAGCCTCTGATTGGAAAGGTGTGGCCTGGATCTACAGCTTTTCCTGATTTTACAAATCCTACAGCTCTGGCTTGGTGGGAG |
| | GATATGGTGGCTGAGTTTCATGATCAGGTGCCTTTTGATGGAATGTGGATTGATATGAATGAGCCTTCTAATTTTATAAGAGGATCTGAGGATGGATGTCCTAATAATGAGCTAGAGAATCCTCCTTATGTGCCTGGAGTAGTGG |
| | GAGGAACACTGCAGGCTGCTACAATTTGTGCTTCTTCTCATCAGTTTCTGTCTACACATTATAATCTGCATAATCTGTATGGACTGACAGAGGCTATTGCTTCTCATAGAGCTCTGGTGAAGGCTAGAGGAACAAGACCTTTTGT |
| | GATTTCTAGATCTACATTTGCTGGACATGGAAGATATGCTGGACATTGGACAGGAGATGTATGGTCTTCTTGGGAGCAGCTAGCTTCTTCTGTGCCTGAGATTCTGCAGTTTAATCTGCTAGGAGTGCCTCTGGTGGGAGCTGAT |
| | GTGTGTGGATTTCTGGGAAATACATCTGAGGAGCTGTGTGTGAGATGGACACAGCTGGGAGCTTTTTATCCTTTTATGAGAAATCATAATTCTCTGCTGTCTCTGCCTCAGGAGCCTTATTCTTTTTCTGAGCCTGCTCAGCAGG |
| | CTATGAGAAAGGCTCTGACACTGAGATATGCTCTGCTGCCTCATCTGTATACACTGTTTCATCAGGCTCATGTGGCTGGAGAGACAGTAGCTAGACCTCTGTTTCTGGAGTTTCCTAAGGATTCTTCTACATGGACAGTTGATCA |
| | TCAGCTGCTGTGGGGAGAGGCTCTGCTGATTACACCTGTGCTGCAGGCTGGAAAGGCTGAGGTGACAGGATATTTTCCTCTGGGAACATGGTATGATCTGCAGACAGTGCCTATAGAGGCTCTGGGATCTCTGCCTCCTCCTCCT |
| | GCTGCTCCTAGAGAGCCTGCTATTCATTCTGAGGGACAGTGGGTGACACTGCCTGCTCCTCTGGATACAATTAATGTGCATCTGAGAGCTGGATATATTATTCCTCTGCAGGGACCTGGACTGACAACAACAGAGTCTAGACAGC |
| | AGCCTATGGCTCTGGCTGTGGCTCTGACAAAGGGAGGAGAGGCTAGAGGAGAGCTGTTTTGGGATGATGGAGAGTCTCTGGAGGTGCTGGAGAGAGGAGCTTATACACAGGTGATTTTTCTGGCTAGAAATAATACAATTGTGAA |
| | TGAGCTGGTGAGAGTGACATCTGAGGGAGCTGGACTGCAGCTGCAGAAGGTGACAGTGCTGGGAGTGGCTACAGCTCCTCAGCAGGTGCTGTCTAATGGAGTGCCTGTGTCTAATTTTACATATTCTCCTGATACAAAGGTGCTG |
| | GATATTTGTGTGTCTCTGCTGATGGGAGAGCAGTTTCTGGTGTCTTGGTGTTAATGATTTAAATCTCGAGCCGGGCGGAGTGTGTTAGTCTCTCCAGAGGGAGGCTGGTTCCCCAGGGAAGCAGAGCCTGTGTGCGGGCAGCAGC |
| | TGTGTGCGGGCCTGGGGGTTGTTAAGTGCAATTATTTTTAATAAAAGGGGCATTTGGAAAAAAAAAAAAAAGGTAGCAGTCGACAGATGAATTCTGCAGATCTGTGGCTTCTAGCTGCCCGGGTGGCATCCCTGTGACCCCTCCC |
| | CAGTGCCTCTCCTGGCCCTGGAAGTTGCCACTCCAGTGCCCACCAGCCTTGTCCTAATAAAATTAAGTTGCATCATTTTGTCTGACTAGGTGTCCTTCTATAATATTATGGGGTGGAGGGGGGTGGTATGGAGCAAGGGGCAAGT |
| | TGGGAAGACAACCTGTAGGGCCTGCGGGGTCTATTGGGAACCAAGCTGGAGTGCAGTGGCACAATCTTGGCTCACTGCAATCTCCGCCTCCTGGGTTCAAGCGATTCTCCTGCCTCAGCCTCCCGAGTTGTTGGGATTCCAGGCA |
| | TGCATGACCAGGCTCAGCTAATTTTTGTTTTTTTGGTAGAGACGGGGTTTCACCATATTGGCCAGGCTGGTCTCCAACTCCTAATCTCAGGTGATCTACCCACCTTGGCCTCCCAAATTGCTGGGATTACAGGCGTGAACCACTG |
| | CTCCCTTCCCTGTCCTTCTGATTTTAAAAAAAAGAGAAATGTTCTGGCACCTGCACTTGCACTGGGGACAGCCTATTTTGCTAGTTTGTTTTGTTTCGTTTTGTTTTGATGGAGAGCGTATGTTGTTTAAACGCGGCCGCGTAGA |
| | TAAGTAGCATGGCGGGTTAATCATTAACTACAAGGAACCCCTAGTGATGGAGTTGGCCACTCCCTCTCTGCGCGCTCGCTCGCTCACTGAGGCCGGGCGACCAAAGGTCGCCCGACGCCCGGGCTTTGCCCGGGCGGCCTCAGTG |
| | AGCGAGCGAGCGCGCAG |
| 25 | > coGAA GAA nucleic acid plus BGH polyA |
| | ATGGGGGTGAGGCACCCTCCCTGTTCTCACAGGCTGCTGGCTGTGTGTGCTCTGGTGTCTCTGGCCACTGCTGCCCTGCTGGGCCATATCCTGCTGCATGACTTCCTGCTGGTGCCTAGGGAGCTGTCTGGCAGCAGCCCTGTGC |
| | TGGAAGAGACCCACCCTGCTCACCAGCAGGGGGCCAGCAGGCCAGGCCCCAGGGATGCCCAGGCCCACCCTGGCAGACCCAGGGCTGTGCCCACCCAGTGTGATGTGCCTCCCAACAGCAGATTTGACTGTGCCCCTGATAAGGC |
| | CATTACTCAGGAACAGTGTGAGGCCAGGGGCTGCTGCTATATCCCTGCCAAGCAGGGCCTGCAGGGGGCCCAGATGGGGCAGCCCTGGTGCTTCTTCCCCCCCAGCTACCCCTCTTATAAGCTGGAGAATCTGAGCAGCTCTGAA |
| | ATGGGCTACACTGCCACTCTGACCAGGACTACCCCCACCTTCTTCCCCAAGGATATTCTGACTCTGAGGCTGGATGTGATGATGGAGACTGAGAACAGGCTGCACTTCACCATCAAGGACCCTGCCAACAGGAGGTATGAGGTGC |
| | CCCTGGAAACTCCCAGGGTGCACTCTAGGGCCCCCAGCCCCCTGTATTCTGTGGAGTTCTCTGAGGAGCCTTTTGGGGTCATTGTCCACAGGCAGCTGGATGGCAGGGTGCTGCTGAATACTACTGTGGCCCCTCTGTTCTTTGC |
| | TGACCAGTTCCTGCAGCTGAGCACCAGCCTGCCTTCTCAGTACATTACTGGCCTGGCTGAGCATCTGAGCCCCCTGATGCTGAGCACCTCTTGGACCAGAATCACCCTGTGGAACAGGGACCTGGCTCCCACTCCTGGGGCCAAC |
| | CTGTATGGCAGCCACCCCTTCTACCTGGCCCTGGAGGATGGGGGCTCTGCCCATGGGGTGTTCCTGCTGAACAGCAATGCCATGGATGTGGTGCTGCAGCCCTCTCCTGCCCTGTCTTGGAGATCTACTGGGGGCATCCTGGATG |
| | TGTATATCTTCCTGGGGCCTGAGCCCAAGTCTGTGGTGCAGCAGTACCTGGATGTGGTGGGCTACCCCTTCATGCCCCCCTACTGGGGCCTGGGCTTCCACCTGTGCAGGTGGGGCTACTCTTCTACTGCTATCACCAGGCAGGT |
| | GGTGGAGAACATGACCAGGGCTCACTTCCCTCTGGATGTGCAGTGGAATGACCTGGACTACATGGACTCTAGGAGAGACTTCACTTTTAATAAGGATGGGTTCAGGGACTTTCCTGCCATGGTGCAGGAGCTGCATCAGGGGGGC |
| | AGGAGATATATGATGATTGTGGACCCTGCTATTTCTAGCTCTGGCCCTGCTGGCAGCTATAGGCCCTATGATGAGGGGCTGAGGAGGGGGGTGTTCATCACTAATGAGACTGGCCAGCCCCTGATTGGCAAGGTGTGGCCTGGCT |
| | CTACTGCCTTCCCTGATTTCACCAACCCCACTGCCCTGGCCTGGTGGGAGGATATGGTGGCTGAGTTTCATGACCAGGTGCCCTTTGATGGCATGTGGATTGACATGAATGAGCCCAGCAACTTTATCAGGGGCTCTGAAGATGG |
| | CTGCCCCAACAATGAGCTGGAGAACCCCCCCTATGTGCCTGGGGTGGTGGGGGGCACCCTGCAGGCTGCCACCATCTGTGCCAGCAGCCACCAGTTCCTGAGCACCCACTATAACCTGCACAACCTGTATGGCCTGACTGAGGCC |
| | ATTGCCTCTCACAGGGCCCTGGTGAAGGCTAGGGGGACTAGGCCCTTTGTGATCAGCAGGTCTACTTTTGCTGGCCATGGCAGGTATGCTGGGCACTGGACTGGGGATGTGTGGTCTAGCTGGGAGCAGCTGGCCAGCTCTGTGC |
| | CTGAGATCCTGCAGTTTAATCTGCTGGGGGTGCCCCTGGTGGGGGCTGATGTGTGTGGCTTCCTGGGCAATACCTCTGAGGAGCTGTGTGTGAGGTGGACTCAGCTGGGGGCTTTCTACCCCTTCATGAGAAACCACAACTCTCT |
| | GCTGAGCCTGCCCCAGGAGCCCTATTCTTTTTCTGAGCCTGCCCAGCAGGCTATGAGGAAGGCCCTGACTCTGAGGTATGCCCTGCTGCCCCACCTGTATACCCTGTTCCATCAGGCCCATGTGGCTGGGGAGACTGTGGCCAGA |
| | CCTCTGTTCCTGGAGTTCCCCAAGGATAGCTCTACTTGGACTGTGGACCACCAGCTGCTGTGGGGGGAGGCTCTGCTGATCACCCCTGTGCTGCAGGCTGGGAAGGCTGAGGTGACTGGCTATTTCCCCCTGGGCACCTGGTATG |
| | ATCTGCAGACTGTGCCCATTGAGGCCCTGGGCTCTCTGCCCCCTCCCCCTGCTGCCCCCAGGGAGCCTGCCATCCACTCTGAGGGCCAGTGGGTGACCCTGCCTGCCCCTCTGGACACTATCAATGTGCACCTGAGGGCTGGCTA |
| | CATCATCCCCCTGCAGGGCCCTGGCCTGACTACCACTGAGTCTAGGCAGCAGCCCATGGCCCTGGCTGTGGCTCTGACCAAGGGGGGGGAGGCCAGGGGGGAGCTGTTCTGGGATGATGGGGAGTCTCTGGAGGTGCTGGAGAGG |
| | GGGGCCTACACCCAGGTGATCTTCCTGGCTAGGAATAACACCATTGTCAATGAGCTGGTGAGGGTGACCTCTGAGGGGGCTGGCCTGCAGCTGCAGAAGGTGACTGTGCTGGGGGTGGCTACTGCCCCCCAGCAGGTGCTGAGCA |
| | ATGGGGTGCCTGTGAGCAACTTCACCTATAGCCCTGACACCAAGGTGCTGGACATCTGTGTGAGCCTGCTGATGGGGGAGCAGTTCCTGGTGAGCTGGTGCTAATTTAAATCTCGAGCCGGGCGGAGTGTGTTAGTCTCTCCAGA |
| | GGGAGGCTGGTTCCCCAGGGAAGCAGAGCCTGTGTGCGGGCAGCAGCTGTGTGCGGGCCTGGGGGTTGTTAAGTGCAATTATTTTTAATAAAAGGGGCATTTGGAAAAAAAAAAAAAAGGTAGCAGTCGACAGATGAATTCTGCA |
| | GATCTGTGGCTTCTAGCTGCCCGGGTGGCATCCCTGTGACCCCTCCCCAGTGCCTCTCCTGGCCCTGGAAGTTGCCACTCCAGTGCCCACCAGCCTTGTCCTAATAAAATTAAGTTGCATCATTTTGTCTGACTAGGTGTCCTTC |
| | TATAATATTATGGGGTGGAGGGGGGTGGTATGGAGCAAGGGGCAAGTTGGGAAGACAACCTGTAGGGCCTGCGGGGTCTATTGGGAACCAAGCTGGAGTGCAGTGGCACAATCTTGGCTCACTGCAATCTCCGCCTCCTGGGTTC |
| | AAGCGATTCTCCTGCCTCAGCCTCCCGAGTTGTTGGGATTCCAGGCATGCATGACCAGGCTCAGCTAATTTTTGTTTTTTTGGTAGAGACGGGGTTTCACCATATTGGCCAGGCTGGTCTCCAACTCCTAATCTCAGGTGATCTA |
| | CCCACCTTGGCCTCCCAAATTGCTGGGATTACAGGCGTGAACCACTGCTCCCTTCCCTGTCCTTCTGATTTTAAAAAAA |
| 26 | > coGAA GAA nucleic acid plus BGH polyA and RevPolII |
| | ATGGGAGTGAGACATCCTCCTTGTTCTCATAGACTGCTGGCTGTGTGTGCTCTGGTGTCTCTGGCTACAGCTGCTCTGCTGGGACATATTCTGCTGCATGATTTTCTGCTAGTGCCTAGAGAGCTGTCTGGATCTTCTCCTGTGCT |
| | GGAGGAGACACATCCTGCTCATCAGCAGGGAGCTTCTAGACCTGGACCTAGAGATGCTCAGGCTCATCCTGGAAGACCTAGAGCTGTGCCTACACAGTGTGATGTGCCTCCTAATTCTAGATTTGATTGTGCTCCTGATAAGGCTA |
| | TAACACAGGAGCAGTGTGAGGCTAGAGGATGTTGTTATATTCCTGCTAAGCAGGGACTGCAGGGAGCTCAGATGGGACAGCCTTGGTGTTTTTTTCCTCCTTCTTATCCTTCTTATAAGCTGGAGAATCTGTCTTCTTCTGAGATG |
| | GGATATACAGCTACACTGACAAGAACAACACCTACATTTTTTCCTAAGGATATTCTGACACTGAGACTGGATGTGATGATGGAGACAGAGAATAGACTGCATTTTACAATTAAGGATCCTGCTAATAGAAGATATGAGGTGCCTCT |
| | AGAGACACCTAGAGTGCATTCTAGAGCTCCTTCTCCTCTGTATTCTGTGGAGTTTTCTGAGGAGCCTTTTGGAGTGATTGTGCATAGACAGCTGGATGGAAGAGTGCTGCTGAATACAACAGTGGCTCCTCTGTTTTTTGCTGATC |
| | AGTTTCTGCAGCTGTCTACATCTCTGCCTTCTCAGTATATTACAGGACTGGCTGAGCATCTGTCTCCTCTGATGCTGTCTACATCTTGGACAAGAATTACACTGTGGAATAGAGATCTGGCTCCTACACCTGGAGCTAATCTGTAT |
| | GGATCTCATCCTTTTTATCTGGCTCTGGAGGATGGAGGATCTGCTCATGGAGTGTTTCTGCTGAATTCTAATGCTATGGATGTAGTGCTGCAGCCTTCTCCTGCTCTGTCTTGGAGATCTACAGGAGGAATTCTGGATGTGTATAT |
| | TTTTCTAGGACCTGAGCCTAAGTCTGTGGTGCAGCAGTATCTGGATGTAGTGGGATATCCTTTTATGCCTCCTTATTGGGGACTGGGATTTCATCTGTGTAGATGGGGATATTCTTCTACAGCTATTACAAGACAGGTGGTTGAGA |
| | ATATGACAAGAGCTCATTTTCCTCTGGATGTGCAGTGGAATGATCTAGATTATATGGATTCTAGAAGAGATTTTACATTTAATAAGGATGGATTTAGAGATTTTCCTGCTATGGTGCAGGAGCTGCATCAGGGAGGAAGAAGATAT |
| | ATGATGATTGTGGATCCTGCTATTTCTTCTTCTGGACCTGCTGGATCTTATAGACCTTATGATGAGGGACTGAGAAGAGGAGTGTTTATTACAAATGAGACAGGACAGCCTCTGATTGGAAAGGTGTGGCCTGGATCTACAGCTTT |
| | TCCTGATTTTACAAATCCTACAGCTCTGGCTTGGTGGGAGGATATGGTGGCTGAGTTTCATGATCAGGTGCCTTTTGATGGAATGTGGATTGATATGAATGAGCCTTCTAATTTTATAAGAGGATCTGAGGATGGATGTCCTAATA |
| | ATGAGCTAGAGAATCCTCCTTATGTGCCTGGAGTAGTGGGAGGAACACTGCAGGCTGCTACAATTTGTGCTTCTTCTCATCAGTTTCTGTCTACACATTATAATCTGCATAATCTGTATGGACTGACAGAGGCTATTGCTTCTCAT |
| | AGAGCTCTGGTGAAGGCTAGAGGAACAAGACCTTTTGTGATTTCTAGATCTACATTTGCTGGACATGGAAGATATGCTGGACATTGGACAGGAGATGTATGGTCTTCTTGGGAGCAGCTAGCTTCTTCTGTGCCTGAGATTCTGCA |
| | GTTTAATCTGCTAGGAGTGCCTCTGGTGGGAGCTGATGTGTGTGGATTTCTGGGAAATACATCTGAGGAGCTGTGTGTGAGATGGACACAGCTGGGAGCTTTTTATCCTTTTATGAGAAATCATAATTCTCTGCTGTCTCTGCCTC |
| | AGGAGCCTTATTCTTTTTCTGAGCCTGCTCAGCAGGCTATGAGAAAGGCTCTGACACTGAGATATGCTCTGCTGCCTCATCTGTATACACTGTTTCATCAGGCTCATGTGGCTGGAGAGACAGTAGCTAGACCTCTGTTTCTGGAG |
| | TTTCCTAAGGATTCTTCTACATGGACAGTTGATCATCAGCTGCTGTGGGGAGAGGCTCTGCTGATTACACCTGTGCTGCAGGCTGGAAAGGCTGAGGTGACAGGATATTTTCCTCTGGGAACATGGTATGATCTGCAGACAGTGCC |
| | TATAGAGGCTCTGGGATCTCTGCCTCCTCCTCCTGCTGCTCCTAGAGAGCCTGCTATTCATTCTGAGGGACAGTGGGTGACACTGCCTGCTCCTCTGGATACAATTAATGTGCATCTGAGAGCTGGATATATTATTCCTCTGCAGG |
| | GACCTGGACTGACAACAACAGAGTCTAGACAGCAGCCTATGGCTCTGGCTGTGGCTCTGACAAAGGGAGGAGAGGCTAGAGGAGAGCTGTTTTGGGATGATGGAGAGTCTCTGGAGGTGCTGGAGAGAGGAGCTTATACACAGGTG |
| | ATTTTTCTGGCTAGAAATAATACAATTGTGAATGAGCTGGTGAGAGTGACATCTGAGGGAGCTGGACTGCAGCTGCAGAAGGTGACAGTGCTGGGAGTGGCTACAGCTCCTCAGCAGGTGCTGTCTAATGGAGTGCCTGTGTCTAA |
| | TTTTACATATTCTCCTGATACAAAGGTGCTGGATATTTGTGTGTCTCTGCTGATGGGAGAGCAGTTTCTGGTGTCTTGGTGTTAATGATTTAAATCTCGAGCCGGGCGGAGTGTGTTAGTCTCTCCAGAGGGAGGCTGGTTCCCCA |
| | GGGAAGCAGAGCCTGTGTGCGGGCAGCAGCTGTGTGCGGGCCTGGGGGTTGTTAAGTGCAATTATTTTTAATAAAAGGGGCATTTGGAAAAAAAAAAAAAAGGTAGCAGTCGACAGATGAATTCTGCAGATCTGTGGCTTCTAGCT |
| | GCCCGGGTGGCATCCCTGTGACCCCTCCCCAGTGCCTCTCCTGGCCCTGGAAGTTGCCACTCCAGTGCCCACCAGCCTTGTCCTAATAAAATTAAGTTGCATCATTTTGTCTGACTAGGTGTCCTTCTATAATATTATGGGGTGGA |
| | GGGGGGTGGTATGGAGCAAGGGGCAAGTTGGGAAGACAACCTGTAGGGCCTGCGGGGTCTATTGGGAACCAAGCTGGAGTGCAGTGGCACAATCTTGGCTCACTGCAATCTCCGCCTCCTGGGTTCAAGCGATTCTCCTGCCTCAG |
| | CCTCCCGAGTTGTTGGGATTCCAGGCATGCATGACCAGGCTCAGCTAATTTTTGTTTTTTTGGTAGAGACGGGGTTTCACCATATTGGCCAGGCTGGTCTCCAACTCCTAATCTCAGGTGATCTACCCACCTTGGCCTCCCAAATT |
| | GCTGGGATTACAGGCGTGAACCACTGCTCCCTTCCCTGTCCTTCTGATTTTAAAAAAAAGAGAAATGTTCTGGCACCTGCACTTGCACTGGGGACAGCCTATTTTGCTAGTTTGTTTTGTTTCGTTTTGTTTTGATGGAGAGCGTA |
| | TGTT |
| 27 | > coGAA plasmid construct |
| | TCTAGAGCTAGCATATGGATCCATCGATTTAGGGATAACAGGGTAATTATCAGCACACAATTGCCCATTATACGCGCGTATAATGGACTATTGTGTGCTGATATCTGTACACTTAAGGGCTAGATCTTAGCTTACGTCACTAGAG |
| | GGTCCACGTTTAGTTTTTAAGATCCATTGATCTCCTAAACGCTGCAAGATTCGCAACCTGGTATACTTAGCCTAGGCGCTAGGTCCTAGTGCAGCGGGACTTTTTTTCTAAAGTCGTTGAGAGGAGGAGTCGTCAGACCAGATAG |
| | CTTTGATGTCCTGATCGGAAGGATCGTTGGCCCCCCTGCAGGCAGCTGTTAATTAACTGCGCGCTCGCTCGCTCACTGAGGCCGCCCGGGCAAAGCCCGGGCGTCGGGCGACCTTTGGTCGCCCGGCCTCAGTGAGCGAGCGAGC |
| | GCGCAGAGAGGGAGTGGCCAACTCCATCACTAGGGGTTCCTTGTAGTTAATGATTAACCCGCCATGCTACTTATCTACGGCGCGCCACGCGTTCTAGGAGTTAATTTTTAAAAAGCAGTCAAAAGTCCAAGTGCCCTTGCGAGCA |
| | TTTACTCTCTCTGTTTGCTCTGGTTAATAATCTCAGGAGCACAAACATTCCTTACTAGTTCTAGGAGTTAATTTTTAAAAAGCAGTCAAAAGTCCAAGTGCCCTTGCGAGCATTTACTCTCTCTGTTTGCTCTGGTTAATAATCT |
| | CAGGAGCACAAACATTCCTTACTAGTTCTAGAGCGGCCGCCAGTGTGCTGGAATTCGGCTTTTTTAGGGCTGGAAGCTACCTTTGACATCATTTCCTCTGCGAATGCATGTATAATTTCTACAGAACCTATTAGAAAGGATCACC |
| | CAGCCTCTGCTTTTGTACAACTTTCCCTTAAAAAACTGCCAATCCCACTGCTGTTTGGCCCAATAGTGAGAACTTTTTCCTGCTGCCTCTTGGTGCTTTTGCCTATGGCCCCTATTCTGCCTGCTGAAGACACTCTTGCCAGCAT |
| | GGACTTAAACCCCTCCAGCTCTGACAATCCTCTTTCTCTTTTGTTTTACATGAAGGGTCTGGCAGCCAAAGCAATCACTCAAAGTTCAAACCTTATCATTTTTTGCTTTGTTCCTCTTGGCCTTGGTTTTGTACATCAGCTTTGA |
| | AAATACCATCCCAGGGTTAATGCTGGGGTTAATTTATAACTGAGAGTGCTCTAGTTCTGCAATACAGGACATGCTATAAAAATGGAAAGATGTTGCTTTCTGAGAGATCAGGTGGATTCTTGGGCATTTGCTTAAGCTTGGTACC |
| | GCCACCATGGGGGTGAGGCACCCTCCCTGTTCTCACAGGCTGCTGGCTGTGTGTGCTCTGGTGTCTCTGGCCACTGCTGCCCTGCTGGGCCATATCCTGCTGCATGACTTCCTGCTGGTGCCTAGGGAGCTGTCTGGCAGCAGCC |
| | CTGTGCTGGAAGAGACCCACCCTGCTCACCAGCAGGGGGCCAGCAGGCCAGGCCCCAGGGATGCCCAGGCCCACCCTGGCAGACCCAGGGCTGTGCCCACCCAGTGTGATGTGCCTCCCAACAGCAGATTTGACTGTGCCCCTGA |
| | TAAGGCCATTACTCAGGAACAGTGTGAGGCCAGGGGCTGCTGCTATATCCCTGCCAAGCAGGGCCTGCAGGGGGCCCAGATGGGGCAGCCCTGGTGCTTCTTCCCCCCCAGCTACCCCTCTTATAAGCTGGAGAATCTGAGCAGC |
| | TCTGAAATGGGCTACACTGCCACTCTGACCAGGACTACCCCCACCTTCTTCCCCAAGGATATTCTGACTCTGAGGCTGGATGTGATGATGGAGACTGAGAACAGGCTGCACTTCACCATCAAGGACCCTGCCAACAGGAGGTATG |
| | AGGTGCCCCTGGAAACTCCCAGGGTGCACTCTAGGGCCCCCAGCCCCCTGTATTCTGTGGAGTTCTCTGAGGAGCCTTTTGGGGTCATTGTCCACAGGCAGCTGGATGGCAGGGTGCTGCTGAATACTACTGTGGCCCCTCTGTT |
| | CTTTGCTGACCAGTTCCTGCAGCTGAGCACCAGCCTGCCTTCTCAGTACATTACTGGCCTGGCTGAGCATCTGAGCCCCCTGATGCTGAGCACCTCTTGGACCAGAATCACCCTGTGGAACAGGGACCTGGCTCCCACTCCTGGG |
| | GCCAACCTGTATGGCAGCCACCCCTTCTACCTGGCCCTGGAGGATGGGGGCTCTGCCCATGGGGTGTTCCTGCTGAACAGCAATGCCATGGATGTGGTGCTGCAGCCCTCTCCTGCCCTGTCTTGGAGATCTACTGGGGGCATCC |
| | TGGATGTGTATATCTTCCTGGGGCCTGAGCCCAAGTCTGTGGTGCAGCAGTACCTGGATGTGGTGGGCTACCCCTTCATGCCCCCCTACTGGGGCCTGGGCTTCCACCTGTGCAGGTGGGGCTACTCTTCTACTGCTATCACCAG |
| | GCAGGTGGTGGAGAACATGACCAGGGCTCACTTCCCTCTGGATGTGCAGTGGAATGACCTGGACTACATGGACTCTAGGAGAGACTTCACTTTTAATAAGGATGGGTTCAGGGACTTTCCTGCCATGGTGCAGGAGCTGCATCAG |
| | GGGGGCAGGAGATATATGATGATTGTGGACCCTGCTATTTCTAGCTCTGGCCCTGCTGGCAGCTATAGGCCCTATGATGAGGGGCTGAGGAGGGGGGTGTTCATCACTAATGAGACTGGCCAGCCCCTGATTGGCAAGGTGTGGC |
| | CTGGCTCTACTGCCTTCCCTGATTTCACCAACCCCACTGCCCTGGCCTGGTGGGAGGATATGGTGGCTGAGTTTCATGACCAGGTGCCCTTTGATGGCATGTGGATTGACATGAATGAGCCCAGCAACTTTATCAGGGGCTCTGA |
| | AGATGGCTGCCCCAACAATGAGCTGGAGAACCCCCCCTATGTGCCTGGGGTGGTGGGGGGCACCCTGCAGGCTGCCACCATCTGTGCCAGCAGCCACCAGTTCCTGAGCACCCACTATAACCTGCACAACCTGTATGGCCTGACT |
| | GAGGCCATTGCCTCTCACAGGGCCCTGGTGAAGGCTAGGGGGACTAGGCCCTTTGTGATCAGCAGGTCTACTTTTGCTGGCCATGGCAGGTATGCTGGGCACTGGACTGGGGATGTGTGGTCTAGCTGGGAGCAGCTGGCCAGCT |
| | CTGTGCCTGAGATCCTGCAGTTTAATCTGCTGGGGGTGCCCCTGGTGGGGGCTGATGTGTGTGGCTTCCTGGGCAATACCTCTGAGGAGCTGTGTGTGAGGTGGACTCAGCTGGGGGCTTTCTACCCCTTCATGAGAAACCACAA |
| | CTCTCTGCTGAGCCTGCCCCAGGAGCCCTATTCTTTTTCTGAGCCTGCCCAGCAGGCTATGAGGAAGGCCCTGACTCTGAGGTATGCCCTGCTGCCCCACCTGTATACCCTGTTCCATCAGGCCCATGTGGCTGGGGAGACTGTG |
| | GCCAGACCTCTGTTCCTGGAGTTCCCCAAGGATAGCTCTACTTGGACTGTGGACCACCAGCTGCTGTGGGGGGAGGCTCTGCTGATCACCCCTGTGCTGCAGGCTGGGAAGGCTGAGGTGACTGGCTATTTCCCCCTGGGCACCT |
| | GGTATGATCTGCAGACTGTGCCCATTGAGGCCCTGGGCTCTCTGCCCCCTCCCCCTGCTGCCCCCAGGGAGCCTGCCATCCACTCTGAGGGCCAGTGGGTGACCCTGCCTGCCCCTCTGGACACTATCAATGTGCACCTGAGGGC |
| | TGGCTACATCATCCCCCTGCAGGGCCCTGGCCTGACTACCACTGAGTCTAGGCAGCAGCCCATGGCCCTGGCTGTGGCTCTGACCAAGGGGGGGGAGGCCAGGGGGGAGCTGTTCTGGGATGATGGGGAGTCTCTGGAGGTGCTG |
| | GAGAGGGGGGCCTACACCCAGGTGATCTTCCTGGCTAGGAATAACACCATTGTCAATGAGCTGGTGAGGGTGACCTCTGAGGGGGCTGGCCTGCAGCTGCAGAAGGTGACTGTGCTGGGGGTGGCTACTGCCCCCCAGCAGGTGC |
| | TGAGCAATGGGGTGCCTGTGAGCAACTTCACCTATAGCCCTGACACCAAGGTGCTGGACATCTGTGTGAGCCTGCTGATGGGGGAGCAGTTCCTGGTGAGCTGGTGCTAATTTAAATCTCGAGCCGGGCGGAGTGTGTTAGTCTC |
| | TCCAGAGGGAGGCTGGTTCCCCAGGGAAGCAGAGCCCTGTGTGCGGGCAGCAGCTGTGTGCGGGCCTGGGGGTTGTTAAGTGCAATTATTTTTAATAAAAGGGGCATTTGGAAAAAAAAAAAAAGGTAGCAGTCGACAGATGAAT |
| | TCTGCAGATCTGTGGCTTCTAGCTGCCCGGGTGGCATCCCTGTGACCCCTCCCCAGTGCCTCTCCTGGCCCTGGAAGTTGCCACTCCAGTGCCCACCAGCCTTGTCCTAATAAAATTAAGTTGCATCATTTTGTCTGACTAGGTG |
| | TCCTTCTATAATATTATGGGGTGGAGGGGGGTGGTATGGAGCAAGGGGCAAGTTGGGAAGACAACCTGTAGGGCCTGCGGGGTCTATTGGGAACCAAGCTGGAGTGCAGTGGCACAATCTTGGCTCACTGCAATCTCCGCCTCCT |
| | GGGTTCAAGCGATTCTCCTGCCTCAGCCTCCCGAGTTGTTGGGATTCCAGGCATGCATGACCAGGCTCAGCTAATTTTTGTTTTTTTGGTAGAGACGGGGTTTCACCATATTGGCCAGGCTGGTCTCCAACTCCTAATCTCAGGT |
| | GATCTACCCACCTTGGCCTCCCAAATTGCTGGGATTACAGGCGTGAACCACTGCTCCCTTCCCTGTCCTTCTGATTTTAAAAAAAAGAGAAATGTTCTGGCACCTGCACTTGCACTGGGGACAGCCTATTTTGCTAGTTTGTTTT |
| | GTTTCGTTTTGTTTTGATGGAGAGCGTATGTTGTTTAAACGCGGCCGCGTAGATAAGTAGCATGGCGGGTTAATCATTAACTACAAGGAACCCCTAGTGATGGAGTTGGCCACTCCCTCTCTGCGCGCTCGCTCGCTCACTGAGG |
| | CCGGGCGACCAAAGGTCGCCCGACGCCCGGGCTTTGCCCGGGCGGCCTCAGTGAGCGAGCGAGCGCGCAGTTAATTAAGGCGCCCTAGGCCGACCCTTAGACTCTGTACTCAGTTCTATAAACGAGCCATTGGATACGAGATCCG |
| | TAGATTGATAAGGGACACGGAATATCCCCGGACGCAATAGACACCGGTGGACAGCTTGGTATCCTGAGCACAGTCGCGCGTCCGAATCTAGCTCTACTTTAGAGGCCCCGGATTCTGATGGTCGTAGACCGCAGAACCGATTGGG |
| | GGGATGAGATCTACTAGTTATCAGCACACAATTGCCCATTATACGCGCGTATAATGGACTATTGTGTGCTGATATAGGGATAACAGGGTAATTCTAGAGCTAGCATATGGATCCATCGATTTGATGCGGTATTTTCTCCTTACGC |
| | ATCTGTGCGGTATTTCACACCGCATACGTCAAAGCAACCATAGTACGCGCCCTGTAGCGGCGCATTAAGCGCGGCGGGTGTGGTGGTTACGCGCAGCGTGACCGCTACACTTGCCAGCGCCCTAGCGCCCGCTCCTTTCGCTTTC |
| | TTCCCTTCCTTTCTCGCCACGTTCGCCGGCTTTCCCCGTCAAGCTCTAAATCGGGGGCTCCCTTTAGGGTTCCGATTTAGTGCTTTACGGCACCTCGACCCCAAAAAACTTGATTTGGGTGATGGTTCACGTAGTGGGCCATCGC |
| | CCTGATAGACGGTTTTTCGCCCTTTGACGTTGGAGTCCACGTTCTTTAATAGTGGACTCTTGTTCCAAACTGGAACAACACTCAACTCTATCTCGGGCTATTCTTTTGATTTATAAGGGATTTTGCCGATTTCGGTCTATTGGTT |
| | AAAAAATGAGCTGATTTAACAAAAATTTAACGCGAATTTTAACAAAATATTAACGTTTACAATTTTATGGTGCACTCTCAGTACAATCTGCTCTGATGCCGCATAGTTAAGCCAGCCCCGACACCCGCCAACACCCGCTGACGCG |
| | CCCTGACGGGCTTGTCTGCTCCCGGCATCCGCTTACAGACAAGCTGTGACCGTCTCCGGGAGCTGCATGTGTCAGAGGTTTTCACCGTCATCACCGAAACGCGCGAGACGAAAGGGCCTCGTGATACGCCTATTTTTATAGGTTA |
| | ATGTCATGATAATAATGGTTTCTTAGACGTCAGGTGGCACTTTTCGGGGAAATGTGCGCGGAACCCCTATTTGTTTATTTTTCTAAATACATTCAAATATGTATCCGCTCATGAGACAATAACCCTGATAAATGCTTCAATAATA |
| | TTGAAAAAGGAAGAGTATGAGCCATATTCAACGGGAAACGTCGAGGCCGCGATTAAATTCCAACATGGATGCTGATTTATATGGGTATAAATGGGCTCGCGATAATGTCGGGCAATCAGGTGCGACAATCTATCGCTTGTATGGG |
| | AAGCCCGATGCGCCAGAGTTGTTTCTGAAACATGGCAAAGGTAGCGTTGCCAATGATGTTACAGATGAGATGGTCAGACTAAACTGGCTGACGGAATTTATGCCTCTTCCGACCATCAAGCATTTTATCCGTACTCCTGATGATG |
| | CATGGTTACTCACCACTGCGATCCCCGGAAAAACAGCATTCCAGGTATTAGAAGAATATCCTGATTCAGGTGAAAATATTGTTGATGCGCTGGCAGTGTTCCTGCGCCGGTTGCATTCGATTCCTGTTTGTAATTGTCCTTTTAA |
| | CAGCGATCGCGTATTTCGTCTCGCTCAGGCGCAATCACGAATGAATAACGGTTTGGTTGATGCGAGTGATTTTGATGACGAGCGTAATGGCTGGCCTGTTGAACAAGTCTGGAAAGAAATGCATAAACTTTTGCCATTCTCACCG |
| | GATTCAGTCGTCACTCATGGTGATTTCTCACTTGATAACCTTATTTTTGACGAGGGGAAATTAATAGGTTGTATTGATGTTGGACGAGTCGGAATCGCAGACCGATACCAGGATCTTGCCATCCTATGGAACTGCCTCGGTGAGT |
| | TTTCTCCTTCATTACAGAAACGGCTTTTTCAAAAATATGGTATTGATAATCCTGATATGAATAAATTGCAGTTTCATTTGATGCTCGATGAGTTTTTCTAAGCGTATAATGGTCTAGAGCTAGCATATGGATCCATCGATTCCAT |
| | TATACGCCTGTCAGACCAAGTTTACTCATATATACTTTAGATTGATTTAAAACTTCATTTTTAATTTAAAAGGATCTAGGTGAAGATCCTTTTTGATAATCTCATGACCAAAATCCCTTAACGTGAGTTTTCGTTCCACTGAGCG |
| | TCAGACCCCGTAGAAAAGATCAAAGGATCTTCTTGAGATCCTTTTTTTCTGCGCGTAATCTGCTGCTTGCAAACAAAAAAACCACCGCTACCAGCGGTGGTTTGTTTGCCGGATCAAGAGCTACCAACTCTTTTTCCGAAGGTAA |
| | CTGGCTTCAGCAGAGCGCAGATACCAAATACTGTTCTTCTAGTGTAGCCGTAGTTAGGCCACCACTTCAAGAACTCTGTAGCACCGCCTACATACCTCGCTCTGCTAATCCTGTTACCAGTGGCTGCTGCCAGTGGCGATAAGTC |
| | GTGTCTTACCGGGTTGGACTCAAGACGATAGTTACCGGATAAGGCGCAGCGGTCGGGCTGAACGGGGGGTTCGTGCACACAGCCCAGCTTGGAGCGAACGACCTACACCGAACTGAGATACCTACAGCGTGAGCTATGAGAAAGC |
| | GCCACGCTTCCCGAAGGGAGAAAGGCGGACAGGTATCCGGTAAGCGGCAGGGTCGGAACAGGAGAGCGCACGAGGGAGCTTCCAGGGGGAAACGCCTGGTATCTTTATAGTCCTGTCGGGTTTCGCCACCTCTGACTTGAGCGTC |
| | GATTTTTGTGATGCTCGTCAGGGGGGCGGAGCCTATGGAAAAACGCCAGCAACGCGGCCTTTTTACGGTTCCTGGCCTTTTGCTGGCCTTTTGCTCACATGT |
| 28 | > coGAAAA plasmid construct |
| | TCTAGAGCTAGCATATGGATCCATCGATTTAGGGATAACAGGGTAATTATCAGCACACAATTGCCCATTATACGCGCGTATAATGGACTATTGTGTGCTGATATCTGTACACTTAAGGGCTAGATCTTAGCTTACGTCACTAGAG |
| | GGTCCACGTTTAGTTTTTAAGATCCATTGATCTCCTAAACGCTGCAAGATTCGCAACCTGGTATACTTAGCCTAGGCGCTAGGTCCTAGTGCAGCGGGACTTTTTTTCTAAAGTCGTTGAGAGGAGGAGTCGTCAGACCAGATAG |
| | CTTTGATGTCCTGATCGGAAGGATCGTTGGCCCCCCTGCAGGCAGCTGTTAATTAACTGCGCGCTCGCTCGCTCACTGAGGCCGCCCGGGCAAAGCCCGGGCGTCGGGCGACCTTTGGTCGCCCGGCCTCAGTGAGCGAGCGAGC |
| | GCGCAGAGAGGGAGTGGCCAACTCCATCACTAGGGGTTCCTTGTAGTTAATGATTAACCCGCCATGCTACTTATCTACGGCGCGCCACGCGTTCTAGGAGTTAATTTTTAAAAAGCAGTCAAAAGTCCAAGTGCCCTTGCGAGCA |
| | TTTACTCTCTCTGTTTGCTCTGGTTAATAATCTCAGGAGCACAAACATTCCTTACTAGTTCTAGGAGTTAATTTTTAAAAAGCAGTCAAAAGTCCAAGTGCCCTTGCGAGCATTTACTCTCTCTGTTTGCTCTGGTTAATAATCT |
| | CAGGAGCACAAACATTCCTTACTAGTTCTAGAGCGGCCGCCAGTGTGCTGGAATTCGGCTTTTTTAGGGCTGGAAGCTACCTTTGACATCATTTCCTCTGCGAATGCATGTATAATTTCTACAGAACCTATTAGAAAGGATCACC |
| | CAGCCTCTGCTTTTGTACAACTTTCCCTTAAAAAACTGCCAATCCCACTGCTGTTTGGCCCAATAGTGAGAACTTTTTCCTGCTGCCTCTTGGTGCTTTTGCCTATGGCCCCTATTCTGCCTGCTGAAGACACTCTTGCCAGCAT |
| | GGACTTAAACCCCTCCAGCTCTGACAATCCTCTTTCTCTTTTGTTTTACATGAAGGGTCTGGCAGCCAAAGCAATCACTCAAAGTTCAAACCTTATCATTTTTTGCTTTGTTCCTCTTGGCCTTGGTTTTGTACATCAGCTTTGA |
| | AAATACCATCCCAGGGTTAATGCTGGGGTTAATTTATAACTGAGAGTGCTCTAGTTCTGCAATACAGGACATGCTATAAAAATGGAAAGATGTTGCTTTCTGAGAGATCAGGTGGATTCTTGGGCATTTGCTTAAGCTTGGTACC |
| | GCCACCATGGGAGTGAGACATCCTCCTTGTTCTCATAGACTGCTGGCTGTGTGTGCTCTGGTGTCTCTGGCTACAGCTGCTCTGCTGGGACATATTCTGCTGCATGATTTTCTGCTAGTGCCTAGAGAGCTGTCTGGATCTTCTC |
| | CTGTGCTGGAGGAGACACATCCTGCTCATCAGCAGGGAGCTTCTAGACCTGGACCTAGAGATGCTCAGGCTCATCCTGGAAGACCTAGAGCTGTGCCTACACAGTGTGATGTGCCTCCTAATTCTAGATTTGATTGTGCTCCTGA |
| | TAAGGCTATAACACAGGAGCAGTGTGAGGCTAGAGGATGTTGTTATATTCCTGCTAAGCAGGGACTGCAGGGAGCTCAGATGGGACAGCCTTGGTGTTTTTTTCCTCCTTCTTATCCTTCTTATAAGCTGGAGAATCTGTCTTCT |
| | TCTGAGATGGGATATACAGCTACACTGACAAGAACAACACCTACATTTTTTCCTAAGGATATTCTGACACTGAGACTGGATGTGATGATGGAGACAGAGAATAGACTGCATTTTACAATTAAGGATCCTGCTAATAGAAGATATG |
| | AGGTGCCTCTAGAGACACCTAGAGTGCATTCTAGAGCTCCTTCTCCTCTGTATTCTGTGGAGTTTTCTGAGGAGCCTTTTGGAGTGATTGTGCATAGACAGCTGGATGGAAGAGTGCTGCTGAATACAACAGTGGCTCCTCTGTT |
| | TTTTGCTGATCAGTTTCTGCAGCTGTCTACATCTCTGCCTTCTCAGTATATTACAGGACTGGCTGAGCATCTGTCTCCTCTGATGCTGTCTACATCTTGGACAAGAATTACACTGTGGAATAGAGATCTGGCTCCTACACCTGGA |
| | GCTAATCTGTATGGATCTCATCCTTTTTATCTGGCTCTGGAGGATGGAGGATCTGCTCATGGAGTGTTTCTGCTGAATTCTAATGCTATGGATGTAGTGCTGCAGCCTTCTCCTGCTCTGTCTTGGAGATCTACAGGAGGAATTC |
| | TGGATGTGTATATTTTTCTAGGACCTGAGCCTAAGTCTGTGGTGCAGCAGTATCTGGATGTAGTGGGATATCCTTTTATGCCTCCTTATTGGGGACTGGGATTTCATCTGTGTAGATGGGGATATTCTTCTACAGCTATTACAAG |
| | ACAGGTGGTTGAGAATATGACAAGAGCTCATTTTCCTCTGGATGTGCAGTGGAATGATCTAGATTATATGGATTCTAGAAGAGATTTTACATTTAATAAGGATGGATTTAGAGATTTTCCTGCTATGGTGCAGGAGCTGCATCAG |
| | GGAGGAAGAAGATATATGATGATTGTGGATCCTGCTATTTCTTCTTCTGGACCTGCTGGATCTTATAGACCTTATGATGAGGGACTGAGAAGAGGAGTGTTTATTACAAATGAGACAGGACAGCCTCTGATTGGAAAGGTGTGGC |
| | CTGGATCTACAGCTTTTCCTGATTTTACAAATCCTACAGCTCTGGCTTGGTGGGAGGATATGGTGGCTGAGTTTCATGATCAGGTGCCTTTTGATGGAATGTGGATTGATATGAATGAGCCTTCTAATTTTATAAGAGGATCTGA |
| | GGATGGATGTCCTAATAATGAGCTAGAGAATCCTCCTTATGTGCCTGGAGTAGTGGGAGGAACACTGCAGGCTGCTACAATTTGTGCTTCTTCTCATCAGTTTCTGTCTACACATTATAATCTGCATAATCTGTATGGACTGACA |
| | GAGGCTATTGCTTCTCATAGAGCTCTGGTGAAGGCTAGAGGAACAAGACCTTTTGTGATTTCTAGATCTACATTTGCTGGACATGGAAGATATGCTGGACATTGGACAGGAGATGTATGGTCTTCTTGGGAGCAGCTAGCTTCTT |
| | CTGTGCCTGAGATTCTGCAGTTTAATCTGCTAGGAGTGCCTCTGGTGGGAGCTGATGTGTGTGGATTTCTGGGAAATACATCTGAGGAGCTGTGTGTGAGATGGACACAGCTGGGAGCTTTTTATCCTTTTATGAGAAATCATAA |
| | TTCTCTGCTGTCTCTGCCTCAGGAGCCTTATTCTTTTTCTGAGCCTGCTCAGCAGGCTATGAGAAAGGCTCTGACACTGAGATATGCTCTGCTGCCTCATCTGTATACACTGTTTCATCAGGCTCATGTGGCTGGAGAGACAGTA |
| | GCTAGACCTCTGTTTCTGGAGTTTCCTAAGGATTCTTCTACATGGACAGTTGATCATCAGCTGCTGTGGGGAGAGGCTCTGCTGATTACACCTGTGCTGCAGGCTGGAAAGGCTGAGGTGACAGGATATTTTCCTCTGGGAACAT |
| | GGTATGATCTGCAGACAGTGCCTATAGAGGCTCTGGGATCTCTGCCTCCTCCTCCTGCTGCTCCTAGAGAGCCTGCTATTCATTCTGAGGGACAGTGGGTGACACTGCCTGCTCCTCTGGATACAATTAATGTGCATCTGAGAGC |
| | TGGATATATTATTCCTCTGCAGGGACCTGGACTGACAACAACAGAGTCTAGACAGCAGCCTATGGCTCTGGCTGTGGCTCTGACAAAGGGAGGAGAGGCTAGAGGAGAGCTGTTTTGGGATGATGGAGAGTCTCTGGAGGTGCTG |
| | GAGAGAGGAGCTTATACACAGGTGATTTTTCTGGCTAGAAATAATACAATTGTGAATGAGCTGGTGAGAGTGACATCTGAGGGAGCTGGACTGCAGCTGCAGAAGGTGACAGTGCTGGGAGTGGCTACAGCTCCTCAGCAGGTGC |
| | TGTCTAATGGAGTGCCTGTGTCTAATTTTACATATTCTCCTGATACAAAGGTGCTGGATATTTGTGTGTCTCTGCTGATGGGAGAGCAGTTTCTGGTGTCTTGGTGTTAATGATTTAAATCTCGAGCCGGGCGGAGTGTGTTAGT |
| | CTCTCCAGAGGGAGGCTGGTTCCCCAGGGAAGCAGAGCCTGTGTGCGGGCAGCAGCTGTGTGCGGGCCTGGGGGTTGTTAAGTGCAATTATTTTTAATAAAAGGGGCATTTGGAAAAAAAAAAAAAAGGTAGCAGTCGACAGATG |
| | AATTCTGCAGATCTGTGGCTTCTAGCTGCCCGGGTGGCATCCCTGTGACCCCTCCCCAGTGCCTCTCCTGGCCCTGGAAGTTGCCACTCCAGTGCCCACCAGCCTTGTCCTAATAAAATTAAGTTGCATCATTTTGTCTGACTAG |
| | GTGTCCTTCTATAATATTATGGGGTGGAGGGGGGTGGCATGGAGCAAGGGGCAAGTTGGGAAGACAACCTGTAGGGCCTGCGGGGTCTATTGGGAACCAAGCTGGAGTGCAGTGGCACAATCTTGGCTCACTGCAATCTCCGCCT |
| | CCTGGGTTCAAGCGATTCTCCTGCCTCAGCCTCCCGAGTTGTTGGGATTCCAGGCATGCATGACCAGGCTCAGCTAATTTTTGTTTTTTTGGTAGAGACGGGGTTTCACCATATTGGCCAGGCTGGTCTCCAACTCCTAATCTCA |
| | GGTGATCTACCCACCTTGGCCTCCCAAATTGCTGGGATTACAGGCGTGAACCACTGCTCCCTTCCCTGTCCTTCTGATTTTAAAAAAAAGAGAAATGTTCTGGCACCTGCACTTGCACTGGGGACAGCCTATTTTGCTAGTTTGT |
| | TTTGTTTCGTTTTGTTTTGATGGAGAGCGTATGTTGTTTAAACGCGGCCGCGTAGATAAGTAGCATGGCGGGTTAATCATTAACTACAAGGAACCCCTAGTGATGGAGTTGGCCACTCCCTCTCTGCGCGCTCGCTCGCTCACTG |
| | AGGCCGGGCGACCAAAGGTCGCCCGACGCCCGGGCTTTGCCCGGGCGGCCTCAGTGAGCGAGCGAGCGCGCAGTTAATTAAGGCGCCCTAGGCCGACCCTTAGACTCTGTACTCAGTTCTATAAACGAGCCATTGGATACGAGAT |
| | CCGTAGATTGATAAGGGACACGGAATATCCCCGGACGCAATAGACACCGGTGGACAGCTTGGTATCCTGAGCACAGTCGCGCGTCCGAATCTAGCTCTACTTTAGAGGCCCCGGATTCTGATGGTCGTAGACCGCAGAACCGATT |
| | GGGGGGATGAGATCTACTAGTTATCAGCACACAATTGCCCATTATACGCGCGTATAATGGACTATTGTGTGCTGATATAGGGATAACAGGGTAATTCTAGAGCTAGCATATGGATCCATCGATTTGATGCGGTATTTTCTCCTTA |
| | CGCATCTGTGCGGTATTTCACACCGCATACGTCAAAGCAACCATAGTACGCGCCCTGTAGCGGCGCATTAAGCGCGGCGGGTGTGGTGGTTACGCGCAGCGTGACCGCTACACTTGCCAGCGCCCTAGCGCCCGCTCCTTTCGCT |
| | TTCTTCCCTTCCTTTCTCGCCACGTTCGCCGGCTTTCCCCGTCAAGCTCTAAATCGGGGGCTCCCTTTAGGGTTCCGATTTAGTGCTTTACGGCACCTCGACCCCAAAAAACTTGATTTGGGTGATGGTTCACGTAGTGGGCCAT |
| | CGCCCTGATAGACGGTTTTTCGCCCTTTGACGTTGGAGTCCACGTTCTTTAATAGTGGACTCTTGTTCCAAACTGGAACAACACTCAACTCTATCTCGGGCTATTCTTTTGATTTATAAGGGATTTTGCCGATTTCGGTCTATTG |
| | GTTAAAAAATGAGCTGATTTAACAAAAATTTAACGCGAATTTTAACAAAATATTAACGTTTACAATTTTATGGTGCACTCTCAGTACAATCTGCTCTGATGCCGCATAGTTAAGCCAGCCCCGACACCCGCCAACACCCGCTGAC |
| | GCGCCCTGACGGGCTTGTCTGCTCCCGGCATCCGCTTACAGACAAGCTGTGACCGTCTCCGGGAGCTGCATGTGTCAGAGGTTTTCACCGTCATCACCGAAACGCGCGAGACGAAAGGGCCTCGTGATACGCCTATTTTTATAGG |
| | TTAATGTCATGATAATAATGGTTTCTTAGACGTCAGGTGGCACTTTTCGGGGAAATGTGCGCGGAACCCCTATTTGTTTATTTTTCTAAATACATTCAAATATGTATCCGCTCATGAGACAATAACCCTGATAAATGCTTCAATA |
| | ATATTGAAAAAGGAAGAGTATGAGCCATATTCAACGGGAAACGTCGAGGCCGCGATTAAATTCCAACATGGATGCTGATTTATATGGGTATAAATGGGCTCGCGATAATGTCGGGCAATCAGGTGCGACAATCTATCGCTTGTAT |
| | GGGAAGCCCGATGCGCCAGAGTTGTTTCTGAAACATGGCAAAGGTAGCGTTGCCAATGATGTTACAGATGAGATGGTCAGACTAAACTGGCTGACGGAATTTATGCCTCTTCCGACCATCAAGCATTTTATCCGTACTCCTGATG |
| | ATGCATGGTTACTCACCACTGCGATCCCCGGAAAAACAGCATTCCAGGTATTAGAAGAATATCCTGATTCAGGTGAAAATATTGTTGATGCGCTGGCAGTGTTCCTGCGCCGGTTGCATTCGATTCCTGTTTGTAATTGTCCTTT |
| | TAACAGCGATCGCGTATTTCGTCTCGCTCAGGCGCAATCACGAATGAATAACGGTTTGGTTGATGCGAGTGATTTTGATGACGAGCGTAATGGCTGGCCTGTTGAACAAGTCTGGAAAGAAATGCATAAACTTTTGCCATTCTCA |
| | CCGGATTCAGTCGTCACTCATGGTGATTTCTCACTTGATAACCTTATTTTTGACGAGGGGAAATTAATAGGTTGTATTGATGTTGGACGAGTCGGAATCGCAGACCGATACCAGGATCTTGCCATCCTATGGAACTGCCTCGGTG |
| | AGTTTTCTCCTTCATTACAGAAACGGCTTTTTCAAAAATATGGTATTGATAATCCTGATATGAATAAATTGCAGTTTCATTTGATGCTCGATGAGTTTTTCTAAGCGTATAATGGTCTAGAGCTAGCATATGGATCCATCGATTC |
| | CATTATACGCCTGTCAGACCAAGTTTACTCATATATACTTTAGATTGATTTAAAACTTCATTTTTAATTTAAAAGGATCTAGGTGAAGATCCTTTTTGATAATCTCATGACCAAAATCCCTTAACGTGAGTTTTCGTTCCACTGA |
| | GCGTCAGACCCCGTAGAAAAGATCAAAGGATCTTCTTGAGATCCTTTTTTTCTGCGCGTAATCTGCTGCTTGCAAACAAAAAAACCACCGCTACCAGCGGTGGTTTGTTTGCCGGATCAAGAGCTACCAACTCTTTTTCCGAAGG |
| | TAACTGGCTTCAGCAGAGCGCAGATACCAAATACTGTTCTTCTAGTGTAGCCGTAGTTAGGCCACCACTTCAAGAACTCTGTAGCACCGCCTACATACCTCGCTCTGCTAATCCTGTTACCAGTGGCTGCTGCCAGTGGCGATAA |
| | GTCGTGTCTTACCGGGTTGGACTCAAGACGATAGTTACCGGATAAGGCGCAGCGGTCGGGCTGAACGGGGGGTTCGTGCACACAGCCCAGCTTGGAGCGAACGACCTACACCGAACTGAGATACCTACAGCGTGAGCTATGAGAA |
| | AGCGCCACGCTTCCCGAAGGGAGAAAGGCGGACAGGTATCCGGTAAGCGGCAGGGTCGGAACAGGAGAGCGCACGAGGGAGCTTCCAGGGGGAAACGCCTGGTATCTTTATAGTCCTGTCGGGTTTCGCCACCTCTGACTTGAGC |
| | GTCGATTTTTGTGATGCTCGTCAGGGGGGCGGAGCCTATGGAAAAACGCCAGCAACGCGGCCTTTTTACGGTTCCTGGCCTTTTGCTGGCCTTTTGCTCACATGT |
| 29 | > coGAA plasmid construct |
| | TCTAGAGCTAGCATATGGATCCATCGATTTAGGGATAACAGGGTAATTATCAGCACACAATTGCCCATTATACGCGCGTATAATGGACTATTGTGTGCTGATATCTGTACACTTAAGGGCTAGATCTTAGCTTACGTCACTAGAG |
| | GGTCCACGTTTAGTTTTTAAGATCCATTGATCTCCTAAACGCTGCAAGATTCGCAACCTGGTATACTTAGCCTAGGCGCTAGGTCCTAGTGCAGCGGGACTTTTTTTCTAAAGTCGTTGAGAGGAGGAGTCGTCAGACCAGATAG |
| | CTTTGATGTCCTGATCGGAAGGATCGTTGGCCCCCCTGCAGGCAGCTGTTAATTAACTGCGCGCTCGCTCGCTCACTGAGGCCGCCCGGGCAAAGCCCGGGCGTCGGGCGACCTTTGGTCGCCCGGCCTCAGTGAGCGAGCGAGC |
| | GCGCAGAGAGGGAGTGGCCAACTCCATCACTAGGGGTTCCTTGTAGTTAATGATTAACCCGCCATGCTACTTATCTACGGCGCGCCACGCGTTCTAGGAGTTAATTTTTAAAAAGCAGTCAAAAGTCCAAGTGCCCTTGCGAGCA |
| | TTTACTCTCTCTGTTTGCTCTGGTTAATAATCTCAGGAGCACAAACATTCCTTACTAGTTCTAGGAGTTAATTTTTAAAAAGCAGTCAAAAGTCCAAGTGCCCTTGCGAGCATTTACTCTCTCTGTTTGCTCTGGTTAATAATCT |
| | CAGGAGCACAAACATTCCTTACTAGTTCTAGAGCGGCCGCCAGTGTGCTGGAATTCGGCTTTTTTAGGGCTGGAAGCTACCTTTGACATCATTTCCTCTGCGAATGCATGTATAATTTCTACAGAACCTATTAGAAAGGATCACC |
| | CAGCCTCTGCTTTTGTACAACTTTCCCTTAAAAAACTGCCAATCCCACTGCTGTTTGGCCCAATAGTGAGAACTTTTTCCTGCTGCCTCTTGGTGCTTTTGCCTATGGCCCCTATTCTGCCTGCTGAAGACACTCTTGCCAGCAT |
| | GGACTTAAACCCCTCCAGCTCTGACAATCCTCTTTCTCTTTTGTTTTACATGAAGGGTCTGGCAGCCAAAGCAATCACTCAAAGTTCAAACCTTATCATTTTTTGCTTTGTTCCTCTTGGCCTTGGTTTTGTACATCAGCTTTGA |
| | AAATACCATCCCAGGGTTAATGCTGGGGTTAATTTATAACTGAGAGTGCTCTAGTTCTGCAATACAGGACATGCTATAAAAATGGAAAGATGTTGCTTTCTGAGAGATCAGGTGGATTCTTGGGCATTTGCTTAAGCTTGGTACC |
| | GCCACCATGGGGGTGAGGCATCCTCCCTGCAGCCATAGGCTGCTGGCTGTGTGTGCCCTGGTGAGCCTGGCTACTGCTGCTCTGCTGGGGCACATCCTGCTGCATGATTTCCTGCTGGTGCCTAGGGAGCTGTCTGGCAGCAGCC |
| | CTGTCCTGGAGGAGACCCACCCTGCCCATCAGCAGGGGGCCTCTAGACCTGGCCCCAGGGATGCCCAGGCCCATCCTGGGAGGCCCAGGGCTGTGCCCACCCAGTGTGATGTGCCTCCCAACAGCAGGTTTGACTGTGCTCCTGA |
| | CAAAGCCATCACCCAGGAGCAGTGTGAAGCCAGGGGCTGTTGTTACATTCCTGCCAAGCAGGGCCTGCAGGGGGCCCAGATGGGCCAGCCCTGGTGCTTCTTCCCCCCTAGCTACCCCAGCTACAAGCTGGAGAATCTGAGCAGC |
| | TCTGAAATGGGCTACACTGCCACCCTGACTAGGACCACCCCCACCTTCTTCCCTAAGGATATCCTGACCCTGAGGCTGGATGTGATGATGGAGACTGAGAACAGGCTGCACTTCACCATCAAGGATCCTGCCAACAGGAGGTATG |
| | AGGTGCCCCTGGAGACCCCCAGGGTGCACAGCAGGGCTCCCTCTCCCCTGTATTCTGTGGAATTCTCTGAGGAGCCCTTTGGGGTGATTGTGCACAGGCAGCTGGATGGCAGAGTGCTGCTGAACACTACTGTGGCTCCCCTGTT |
| | CTTTGCTGATCAGTTCCTGCAGCTGAGCACTTCTCTGCCCAGCCAGTACATCACTGGGCTGGCTGAGCACCTGAGCCCTCTGATGCTGAGCACCAGCTGGACCAGGATCACCCTGTGGAACAGGGACCTGGCTCCCACCCCTGGG |
| | GCCAATCTGTATGGCAGCCACCCCTTCTACCTGGCTCTGGAGGATGGGGGCTCTGCCCATGGGGTGTTCCTGCTGAACTCTAATGCCATGGATGTGGTGCTGCAGCCCAGCCCTGCCCTGAGCTGGAGGAGCACTGGGGGCATCC |
| | TGGATGTGTACATCTTCCTGGGCCCTGAGCCCAAGTCTGTGGTCCAGCAGTATCTGGATGTGGTGGGGTACCCTTTCATGCCCCCCTACTGGGGCCTGGGCTTCCACCTGTGCAGGTGGGGGTACAGCAGCACTGCCATCACCAG |
| | GCAGGTGGTGGAGAATATGACCAGGGCCCACTTCCCCCTGGATGTGCAGTGGAATGATCTGGACTACATGGATTCTAGGAGGGACTTCACCTTCAACAAGGATGGCTTCAGGGACTTTCCTGCCATGGTGCAGGAGCTGCACCAG |
| | GGGGGCAGGAGGTACATGATGATTGTGGACCCAGCCATCTCTAGCTCTGGGCCAGCTGGCAGCTACAGGCCCTATGATGAGGGCCTGAGGAGGGGGGTGTTCATCACTAATGAGACTGGCCAGCCCCTGATTGGCAAGGTGTGGC |
| | CTGGGAGCACTGCCTTCCCTGACTTCACCAACCCCACTGCCCTGGCCTGGTGGGAGGATATGGTGGCTGAGTTCCATGACCAGGTGCCCTTTGATGGGATGTGGATTGACATGAATGAGCCCTCTAACTTTATTAGGGGCTCTGA |
| | GGATGGGTGCCCTAACAATGAGCTGGAGAACCCCCCTTATGTGCCTGGGGTGGTGGGGGGCACCCTGCAGGCTGCCACCATCTGTGCCAGCAGCCACCAGTTCCTGTCTACCCACTACAACCTGCACAATCTGTATGGGCTGACT |
| | GAGGCTATTGCCAGCCACAGGGCCCTGGTGAAGGCTAGGGGGACCAGGCCTTTTGTGATCAGCAGGAGCACCTTTGCTGGGCATGGCAGATATGCTGGCCATTGGACTGGGGATGTGTGGTCTAGCTGGGAGCAGCTGGCCAGCT |
| | CTGTGCCTGAGATCCTGCAGTTCAATCTGCTGGGGGTGCCCCTGGTGGGGGCTGATGTGTGTGGGTTCCTGGGCAATACCTCTGAGGAGCTGTGTGTGAGGTGGACCCAGCTGGGGGCCTTCTACCCCTTCATGAGGAACCACAA |
| | CAGCCTGCTGAGCCTGCCTCAGGAGCCCTATTCTTTTTCTGAGCCTGCCCAGCAGGCTATGAGGAAGGCCCTGACTCTGAGGTATGCCCTGCTGCCTCACCTGTACACCCTGTTCCACCAGGCCCATGTGGCTGGGGAGACTGTG |
| | GCCAGGCCCCTGTTCCTGGAGTTCCCCAAGGATAGCTCTACCTGGACTGTGGACCATCAGCTGCTGTGGGGGGAGGCCCTGCTGATCACCCCTGTGCTGCAGGCTGGCAAGGCTGAGGTGACTGGCTACTTTCCTCTGGGCACCT |
| | GGTATGATCTGCAGACTGTGCCCATTGAAGCCCTGGGCAGCCTGCCTCCTCCCCCTGCTGCCCCTAGGGAGCCTGCCATCCACTCTGAGGGCCAGTGGGTGACCCTGCCAGCCCCTCTGGACACCATCAATGTGCATCTGAGGGC |
| | TGGCTACATCATTCCCCTGCAGGGCCCTGGGCTGACCACCACTGAGAGCAGGCAGCAGCCCATGGCTCTGGCTGTGGCCCTGACCAAGGGGGGGGAGGCCAGGGGGGAGCTGTTCTGGGATGATGGGGAGAGCCTGGAGGTGCTG |
| | GAGAGGGGGGCCTACACCCAGGTGATCTTCCTGGCCAGGAATAATACCATTGTGAATGAGCTGGTGAGGGTGACTTCTGAGGGGGCTGGCCTGCAGCTGCAGAAGGTGACTGTGCTGGGGGTGGCTACTGCTCCTCAGCAGGTGC |
| | TGTCTAATGGGGTGCCTGTGAGCAACTTCACTTACAGCCCTGACACTAAGGTGCTGGACATTTGTGTGAGCCTGCTGATGGGGGAGCAGTTCCTGGTGTCTTGGTGCTGATTTAAATCTCGAGCCGGGCGGAGTGTGTTAGTCTC |
| | TCCAGAGGGAGGCTGGTTCCCCAGGGAAGCAGAGCCTGTGTGCGGGCAGCAGCTGTGTGCGGGCCTGGGGGTTGTTAAGTGCAATTATTTTTAATAAAAGGGGCATTTGGAAAAAAAAAAAAAAGGTAGCAGTCGACAGATGAAT |
| | TCTGCAGATCTGTGGCTTCTAGCTGCCCGGGTGGCATCCCTGTGACCCCTCCCCAGTGCCTCTCCTGGCCCTGGAAGTTGCCACTCCAGTGCCCACCAGCCTTGTCCTAATAAAATTAAGTTGCATCATTTTGTCTGACTAGGTG |
| | TCCTTCTATAATATTATGGGGTGGAGGGGGGTGGTATGGAGCAAGGGGCAAGTTGGGAAGACAACCTGTAGGGCCTGCGGGGTCTATTGGGAACCAAGCTGGAGTGCAGTGGCACAATCTTGGCTCACTGCAATCTCCGCCTCCT |
| | GGGTTCAAGCGATTCTCCTGCCTCAGCCTCCCGAGTTGTTGGGATTCCAGGCATGCATGACCAGGCTCAGCTAATTTTTGTTTTTTTGGTAGAGACGGGGTTTCACCATATTGGCCAGGCTGGTCTCCAACTCCTAATCTCAGGT |
| | GATCTACCCACCTTGGCCTCCCAAATTGCTGGGATTACAGGCGTGAACCACTGCTCCCTTCCCTGTCCTTCTGATTTTAAAAAAAAGAGAAATGTTCTGGCACCTGCACTTGCACTGGGGACAGCCTATTTTGCTAGTTTGTTTT |
| | GTTTCGTTTTGTTTTGATGGAGAGCGTATGTTGTTTAAACGCGGCCGCGTAGATAAGTAGCATGGCGGGTTAATCATTAACTACAAGGAACCCCTAGTGATGGAGTTGGCCACTCCCTCTCTGCGCGCTCGCTCGCTCACTGAGG |
| | CCGGGCGACCAAAGGTCGCCCGACGCCCGGGCTTTGCCCGGGCGGCCTCAGTGAGCGAGCGAGCGCGCAGTTAATTAAGGCGCCCTAGGCCGACCCTTAGACTCTGTACTCAGTTCTATAAACGAGCCATTGGATACGAGATCCG |
| | TAGATTGATAAGGGACACGGAATATCCCCGGACGCAATAGACACCGGTGGACAGCTTGGTATCCTGAGCACAGTCGCGCGTCCGAATCTAGCTCTACTTTAGAGGCCCCGGATTCTGATGGTCGTAGACCGCAGAACCGATTGGG |
| | GGGATGAGATCTACTAGTTATCAGCACACAATTGCCCATTATACGCGCGTATAATGGACTATTGTGTGCTGATATAGGGATAACAGGGTAATTCTAGAGCTAGCATATGGATCCATCGATTTGATGCGGTATTTTCTCCTTACGC |
| | ATCTGTGCGGTATTTCACACCGCATACGTCAAAGCAACCATAGTACGCGCCCTGTAGCGGCGCATTAAGCGCGGCGGGTGTGGTGGTTACGCGCAGCGTGACCGCTACACTTGCCAGCGCCCTAGCGCCCGCTCCTTTCGCTTTC |
| | TTCCCTTCCTTTCTCGCCACGTTCGCCGGCTTTCCCCGTCAAGCTCTAAATCGGGGGCTCCCTTTAGGGTTCCGATTTAGTGCTTTACGGCACCTCGACCCCAAAAAACTTGATTTGGGTGATGGTTCACGTAGTGGGCCATCGC |
| | CCTGATAGACGGTTTTTCGCCCTTTGACGTTGGAGTCCACGTTCTTTAATAGTGGACTCTTGTTCCAAACTGGAACAACACTCAACTCTATCTCGGGCTATTCTTTTGATTTATAAGGGATTTTGCCGATTTCGGTCTATTGGTT |
| | AAAAAATGAGCTGATTTAACAAAAATTTAACGCGAATTTTAACAAAATATTAACGTTTACAATTTTATGGTGCACTCTCAGTACAATCTGCTCTGATGCCGCATAGTTAAGCCAGCCCCGACACCCGCCAACACCCGCTGACGCG |
| | CCCTGACGGGCTTGTCTGCTCCCGGCATCCGCTTACAGACAAGCTGTGACCGTCTCCGGGAGCTGCATGTGTCAGAGGTTTTCACCGTCATCACCGAAACGCGCGAGACGAAAGGGCCTCGTGATACGCCTATTTTTATAGGTTA |
| | ATGTCATGATAATAATGGTTTCTTAGACGTCAGGTGGCACTTTTCGGGGAAATGTGCGCGGAACCCCTATTTGTTTATTTTTCTAAATACATTCAAATATGTATCCGCTCATGAGACAATAACCCTGATAAATGCTTCAATAATA |
| | TTGAAAAAGGAAGAGTATGAGCCATATTCAACGGGAAACGTCGAGGCCGCGATTAAATTCCAACATGGATGCTGATTTATATGGGTATAAATGGGCTCGCGATAATGTCGGGCAATCAGGTGCGACAATCTATCGCTTGTATGGG |
| | AAGCCCGATGCGCCAGAGTTGTTTCTGAAACATGGCAAAGGTAGCGTTGCCAATGATGTTACAGATGAGATGGTCAGACTAAACTGGCTGACGGAATTTATGCCTCTTCCGACCATCAAGCATTTTATCCGTACTCCTGATGATG |
| | CATGGTTACTCACCACTGCGATCCCCGGAAAAACAGCATTCCAGGTATTAGAAGAATATCCTGATTCAGGTGAAAATATTGTTGATGCGCTGGCAGTGTTCCTGCGCCGGTTGCATTCGATTCCTGTTTGTAATTGTCCTTTTAA |
| | CAGCGATCGCGTATTTCGTCTCGCTCAGGCGCAATCACGAATGAATAACGGTTTGGTTGATGCGAGTGATTTTGATGACGAGCGTAATGGCTGGCCTGTTGAACAAGTCTGGAAAGAAATGCATAAACTTTTGCCATTCTCACCG |
| | GATTCAGTCGTCACTCATGGTGATTTCTCACTTGATAACCTTATTTTTGACGAGGGGAAATTAATAGGTTGTATTGATGTTGGACGAGTCGGAATCGCAGACCGATACCAGGATCTTGCCATCCTATGGAACTGCCTCGGTGAGT |
| | TTTCTCCTTCATTACAGAAACGGCTTTTTCAAAAATATGGTATTGATAATCCTGATATGAATAAATTGCAGTTTCATTTGATGCTCGATGAGTTTTTCTAAGCGTATAATGGTCTAGAGCTAGCATATGGATCCATCGATTCCAT |
| | TATACGCCTGTCAGACCAAGTTTACTCATATATACTTTAGATTGATTTAAAACTTCATTTTTAATTTAAAAGGATCTAGGTGAAGATCCTTTTTGATAATCTCATGACCAAAATCCCTTAACGTGAGTTTTCGTTCCACTGAGCG |
| | TCAGACCCCGTAGAAAAGATCAAAGGATCTTCTTGAGATCCTTTTTTTCTGCGCGTAATCTGCTGCTTGCAAACAAAAAAACCACCGCTACCAGCGGTGGTTTGTTTGCCGGATCAAGAGCTACCAACTCTTTTTCCGAAGGTAA |
| | CTGGCTTCAGCAGAGCGCAGATACCAAATACTGTTCTTCTAGTGTAGCCGTAGTTAGGCCACCACTTCAAGAACTCTGTAGCACCGCCTACATACCTCGCTCTGCTAATCCTGTTACCAGTGGCTGCTGCCAGTGGCGATAAGTC |
| | GTGTCTTACCGGGTTGGACTCAAGACGATAGTTACCGGATAAGGCGCAGCGGTCGGGCTGAACGGGGGGTTCGTGCACACAGCCCAGCTTGGAGCGAACGACCTACACCGAACTGAGATACCTACAGCGTGAGCTATGAGAAAGC |
| | GCCACGCTTCCCGAAGGGAGAAAGGCGGACAGGTATCCGGTAAGCGGCAGGGTCGGAACAGGAGAGCGCACGAGGGAGCTTCCAGGGGGAAACGCCTGGTATCTTTATAGTCCTGTCGGGTTTCGCCACCTCTGACTTGAGCGTC |
| | GATTTTTGTGATGCTCGTCAGGGGGGCGGAGCCTATGGAAAAACGCCAGCAACGCGGCCTTTTTACGGTTCCTGGCCTTTTGCTGGCCTTTTGCTCACATGT |
| 30 | > coGAA plasmid construct |
| | TCTAGAGCTAGCATATGGATCCATCGATTTAGGGATAACAGGGTAATTATCAGCACACAATTGCCCATTATACGCGCGTATAATGGACTATTGTGTGCTGATATCTGTACACTTAAGGGCTAGATCTTAGCTTACGTCACTAGAG |
| | GGTCCACGTTTAGTTTTTAAGATCCATTGATCTCCTAAACGCTGCAAGATTCGCAACCTGGTATACTTAGCCTAGGCGCTAGGTCCTAGTGCAGCGGGACTTTTTTTCTAAAGTCGTTGAGAGGAGGAGTCGTCAGACCAGATAG |
| | CTTTGATGTCCTGATCGGAAGGATCGTTGGCCCCCCTGCAGGCAGCTGTTAATTAACTGCGCGCTCGCTCGCTCACTGAGGCCGCCCGGGCAAAGCCCGGGCGTCGGGCGACCTTTGGTCGCCCGGCCTCAGTGAGCGAGCGAGC |
| | GCGCAGAGAGGGAGTGGCCAACTCCATCACTAGGGGTTCCTTGTAGTTAATGATTAACCCGCCATGCTACTTATCTACGGCGCGCCACGCGTTCTAGGAGTTAATTTTTAAAAAGCAGTCAAAAGTCCAAGTGCCCTTGCGAGCA |
| | TTTACTCTCTCTGTTTGCTCTGGTTAATAATCTCAGGAGCACAAACATTCCTTACTAGTTCTAGGAGTTAATTTTTAAAAAGCAGTCAAAAGTCCAAGTGCCCTTGCGAGCATTTACTCTCTCTGTTTGCTCTGGTTAATAATCT |
| | CAGGAGCACAAACATTCCTTACTAGTTCTAGAGCGGCCGCCAGTGTGCTGGAATTCGGCTTTTTTAGGGCTGGAAGCTACCTTTGACATCATTTCCTCTGCGAATGCATGTATAATTTCTACAGAACCTATTAGAAAGGATCACC |
| | CAGCCTCTGCTTTTGTACAACTTTCCCTTAAAAAACTGCCAATCCCACTGCTGTTTGGCCCAATAGTGAGAACTTTTTCCTGCTGCCTCTTGGTGCTTTTGCCTATGGCCCCTATTCTGCCTGCTGAAGACACTCTTGCCAGCAT |
| | GGACTTAAACCCCTCCAGCTCTGACAATCCTCTTTCTCTTTTGTTTTACATGAAGGGTCTGGCAGCCAAAGCAATCACTCAAAGTTCAAACCTTATCATTTTTTGCTTTGTTCCTCTTGGCCTTGGTTTTGTACATCAGCTTTGA |
| | AAATACCATCCCAGGGTTAATGCTGGGGTTAATTTATAACTGAGAGTGCTCTAGTTCTGCAATACAGGACATGCTATAAAAATGGAAAGATGTTGCTTTCTGAGAGATCAGGTGGATTCTTGGGCATTTGCTTAAGCTTGGTACC |
| | GCCACCATGGGGGTGAGGCACCCCCCCTGCTCTCACAGGCTGCTGGCTGTGTGTGCCCTGGTGAGCCTGGCCACTGCTGCTCTGCTGGGGCACATCCTGCTGCATGACTTCCTGCTGGTGCCCAGGGAGCTGTCTGGCAGCTCTC |
| | CTGTGCTGGAGGAAACTCATCCTGCCCATCAGCAGGGGGCCTCTAGGCCAGGCCCCAGGGATGCCCAGGCCCATCCTGGCAGGCCCAGAGCTGTGCCCACCCAGTGTGATGTGCCCCCCAACAGCAGGTTTGATTGTGCCCCTGA |
| | TAAGGCCATCACCCAGGAGCAGTGTGAGGCCAGGGGCTGCTGCTACATCCCTGCCAAGCAGGGGCTGCAGGGGGCCCAGATGGGCCAGCCCTGGTGCTTCTTCCCCCCTTCTTACCCCAGCTACAAGCTGGAGAACCTGTCTTCT |
| | TCTGAGATGGGCTATACTGCCACCCTGACCAGGACCACTCCCACCTTCTTCCCCAAGGACATCCTGACTCTGAGACTGGATGTGATGATGGAGACTGAGAACAGGCTGCACTTCACCATCAAGGACCCTGCCAACAGGAGGTATG |
| | AGGTGCCTCTGGAGACTCCTAGAGTGCACAGCAGGGCTCCCTCTCCCCTGTACTCTGTGGAGTTCTCTGAGGAGCCCTTTGGGGTGATTGTGCACAGACAGCTGGATGGGAGGGTGCTGCTGAACACCACTGTGGCCCCCCTGTT |
| | CTTTGCTGACCAGTTCCTGCAGCTGTCTACCTCTCTGCCCAGCCAGTACATTACTGGGCTGGCTGAACATCTGTCTCCCCTGATGCTGAGCACCAGCTGGACCAGGATCACTCTGTGGAACAGGGACCTGGCCCCCACTCCTGGG |
| | GCCAATCTGTATGGGAGCCACCCATTCTACCTGGCCCTGGAGGATGGGGGCTCTGCCCATGGGGTGTTCCTGCTGAACAGCAATGCCATGGATGTGGTGCTGCAGCCTTCCCCTGCCCTGAGCTGGAGGTCTACTGGGGGCATCC |
| | TGGATGTGTACATTTTCCTGGGCCCTGAGCCCAAGTCTGTGGTGCAGCAGTACCTGGATGTGGTGGGGTACCCTTTCATGCCTCCCTACTGGGGCCTGGGCTTTCACCTGTGCAGGTGGGGCTATTCTAGCACTGCCATCACCAG |
| | GCAGGTGGTGGAGAACATGACTAGGGCTCATTTCCCCCTGGATGTGCAGTGGAATGATCTGGATTATATGGACTCTAGGAGGGACTTTACTTTCAATAAGGATGGCTTCAGGGATTTTCCTGCTATGGTGCAGGAGCTGCACCAG |
| | GGGGGCAGGAGGTATATGATGATTGTGGATCCTGCCATCTCTAGCTCTGGGCCTGCTGGCTCTTACAGGCCCTATGATGAGGGGCTGAGGAGGGGGGTGTTTATTACTAATGAGACTGGCCAGCCTCTGATTGGGAAGGTGTGGC |
| | CTGGCAGCACTGCCTTCCCAGACTTCACCAACCCCACTGCCCTGGCCTGGTGGGAGGACATGGTGGCTGAGTTCCATGACCAGGTGCCCTTTGATGGGATGTGGATTGATATGAATGAGCCCTCTAACTTCATTAGGGGCTCTGA |
| | GGATGGCTGCCCCAACAATGAGCTGGAGAACCCCCCATATGTGCCTGGGGTGGTGGGGGGGACCCTGCAGGCTGCTACCATCTGTGCTAGCTCTCACCAGTTCCTGAGCACCCACTACAACCTGCACAATCTGTATGGCCTGACT |
| | GAGGCCATTGCCAGCCATAGGGCCCTGGTGAAGGCCAGGGGGACCAGACCCTTTGTGATCTCCAGGAGCACCTTTGCTGGCCATGGGAGATATGCTGGCCATTGGACTGGGGATGTGTGGTCTAGCTGGGAGCAGCTGGCCAGCT |
| | CTGTGCCTGAGATCCTGCAGTTCAATCTGCTGGGGGTGCCTCTGGTGGGGGCTGATGTGTGTGGCTTCCTGGGCAACACCTCTGAAGAACTGTGTGTGAGATGGACCCAGCTGGGGGCCTTCTACCCCTTCATGAGGAACCACAA |
| | CAGCCTGCTGTCTCTGCCCCAGGAGCCTTACTCTTTTTCTGAGCCTGCCCAGCAGGCCATGAGGAAGGCCCTGACCCTGAGATATGCCCTGCTGCCTCACCTGTACACCCTGTTCCACCAGGCCCATGTGGCTGGGGAGACTGTG |
| | GCCAGGCCCCTGTTCCTGGAGTTCCCCAAGGACAGCAGCACCTGGACTGTGGACCACCAGCTGCTGTGGGGGGAGGCCCTGCTGATCACCCCTGTGCTGCAGGCTGGGAAGGCTGAGGTGACTGGCTACTTCCCCCTGGGCACCT |
| | GGTATGATCTGCAGACTGTGCCCATTGAGGCCCTGGGGTCTCTGCCCCCCCCCCCTGCTGCTCCCAGAGAGCCTGCCATCCATTCTGAGGGCCAGTGGGTGACCCTGCCTGCCCCCCTGGACACCATTAATGTGCACCTGAGGGC |
| | TGGCTACATTATCCCCCTGCAGGGCCCTGGCCTGACCACCACTGAGAGCAGGCAGCAGCCTATGGCTCTGGCTGTGGCCCTGACCAAGGGGGGGGAGGCCAGGGGGGAGCTGTTTTGGGATGATGGGGAGAGCCTGGAGGTGCTG |
| | GAGAGGGGGGCCTACACCCAGGTGATCTTCCTGGCCAGGAACAACACTATTGTGAATGAACTGGTGAGGGTCACTTCTGAGGGGGCTGGGCTGCAGCTGCAGAAGGTGACTGTGCTGGGGGTGGCCACTGCTCCCCAGCAGGTGC |
| | TGAGCAATGGGGTGCCTGTGAGCAACTTCACCTACAGCCCTGATACCAAGGTGCTGGACATCTGTGTGAGCCTGCTGATGGGGGAGCAGTTCCTGGTGAGCTGGTGCTGATTTAAATCTCGAGCCGGGCGGAGTGTGTTAGTCTC |
| | TCCAGAGGGAGGCTGGTTCCCCAGGGAAGCAGAGCCTGTGTGCGGGCAGCAGCTGTGTGCGGGCCTGGGGGTTGTTAAGTGCAATTATTTTTAATAAAAGGGGCATTTGGAAAAAAAAAAAAAAGGTAGCAGTCGACAGATGAAT |
| | TCTGCAGATCTGTGGCTTCTAGCTGCCCGGGTGGCATCCCTGTGACCCCTCCCCAGTGCCTCTCCTGGCCCTGGAAGTTGCCACTCCAGTGCCCACCAGCCTTGTCCTAATAAAATTAAGTTGCATCATTTTGTCTGACTAGGTG |
| | TCCTTCTATAATATTATGGGGTGGAGGGGGGTGGTATGGAGCAAGGGGCAAGTTGGGAAGACAACCTGTAGGGCCTGCGGGGTCTATTGGGAACCAAGCTGGAGTGCAGTGGCACAATCTTGGCTCACTGCAATCTCCGCCTCCT |
| | GGGTTCAAGCGATTCTCCTGCCTCAGCCTCCCGAGTTGTTGGGATTCCAGGCATGCATGACCAGGCTCAGCTAATTTTTGTTTTTTTGGTAGAGACGGGGTTTCACCATATTGGCCAGGCTGGTCTCCAACTCCTAATCTCAGGT |
| | GATCTACCCACCTTGGCCTCCCAAATTGCTGGGATTACAGGCGTGAACCACTGCTCCCTTCCCTGTCCTTCTGATTTTAAAAAAAAGAGAAATGTTCTGGCACCTGCACTTGCACTGGGGACAGCCTATTTTGCTAGTTTGTTTT |
| | GTTTCGTTTTGTTTTGATGGAGAGCGTATGTTGTTTAAACGCGGCCGCGTAGATAAGTAGCATGGCGGGTTAATCATTAACTACAAGGAACCCCTAGTGATGGAGTTGGCCACTCCCTCTCTGCGCGCTCGCTCGCTCACTGAGG |
| | CCGGGCGACCAAAGGTCGCCCGACGCCCGGGCTTTGCCCGGGCGGCCTCAGTGAGCGAGCGAGCGCGCAGTTAATTAAGGCGCCCTAGGCCGACCCTTAGACTCTGTACTCAGTTCTATAAACGAGCCATTGGATACGAGATCCG |
| | TAGATTGATAAGGGACACGGAATATCCCCGGACGCAATAGACACCGGTGGACAGCTTGGTATCCTGAGCACAGTCGCGCGTCCGAATCTAGCTCTACTTTAGAGGCCCCGGATTCTGATGGTCGTAGACCGCAGAACCGATTGGG |
| | GGGATGAGATCTACTAGTTATCAGCACACAATTGCCCATTATACGCGCGTATAATGGACTATTGTGTGCTGATATAGGGATAACAGGGTAATTCTAGAGCTAGCATATGGATCCATCGATTTGATGCGGTATTTTCTCCTTACGC |
| | ATCTGTGCGGTATTTCACACCGCATACGTCAAAGCAACCATAGTACGCGCCCTGTAGCGGCGCATTAAGCGCGGCGGGTGTGGTGGTTACGCGCAGCGTGACCGCTACACTTGCCAGCGCCCTAGCGCCCGCTCCTTTCGCTTTC |
| | TTCCCTTCCTTTCTCGCCACGTTCGCCGGCTTTCCCCGTCAAGCTCTAAATCGGGGGCTCCCTTTAGGGTTCCGATTTAGTGCTTTACGGCACCTCGACCCCAAAAAACTTGATTTGGGTGATGGTTCACGTAGTGGGCCATCGC |
| | CCTGATAGACGGTTTTTCGCCCTTTGACGTTGGAGTCCACGTTCTTTAATAGTGGACTCTTGTTCCAAACTGGAACAACACTCAACTCTATCTCGGGCTATTCTTTTGATTTATAAGGGATTTTGCCGATTTCGGTCTATTGGTT |
| | AAAAAATGAGCTGATTTAACAAAAATTTAACGCGAATTTTAACAAAATATTAACGTTTACAATTTTATGGTGCACTCTCAGTACAATCTGCTCTGATGCCGCATAGTTAAGCCAGCCCCGACACCCGCCAACACCCGCTGACGCG |
| | CCCTGACGGGCTTGTCTGCTCCCGGCATCCGCTTACAGACAAGCTGTGACCGTCTCCGGGAGCTGCATGTGTCAGAGGTTTTCACCGTCATCACCGAAACGCGCGAGACGAAAGGGCCTCGTGATACGCCTATTTTTATAGGTTA |
| | ATGTCATGATAATAATGGTTTCTTAGACGTCAGGTGGCACTTTTCGGGGAAATGTGCGCGGAACCCCTATTTGTTTATTTTTCTAAATACATTCAAATATGTATCCGCTCATGAGACAATAACCCTGATAAATGCTTCAATAATA |
| | TTGAAAAAGGAAGAGTATGAGCCATATTCAACGGGAAACGTCGAGGCCGCGATTAAATTCCAACATGGATGCTGATTTATATGGGTATAAATGGGCTCGCGATAATGTCGGGCAATCAGGTGCGACAATCTATCGCTTGTATGGG |
| | AAGCCCGATGCGCCAGAGTTGTTTCTGAAACATGGCAAAGGTAGCGTTGCCAATGATGTTACAGATGAGATGGTCAGACTAAACTGGCTGACGGAATTTATGCCTCTTCCGACCATCAAGCATTTTATCCGTACTCCTGATGATG |
| | CATGGTTACTCACCACTGCGATCCCCGGAAAAACAGCATTCCAGGTATTAGAAGAATATCCTGATTCAGGTGAAAATATTGTTGATGCGCTGGCAGTGTTCCTGCGCCGGTTGCATTCGATTCCTGTTTGTAATTGTCCTTTTAA |
| | CAGCGATCGCGTATTTCGTCTCGCTCAGGCGCAATCACGAATGAATAACGGTTTGGTTGATGCGAGTGATTTTGATGACGAGCGTAATGGCTGGCCTGTTGAACAAGTCTGGAAAGAAATGCATAAACTTTTGCCATTCTCACCG |
| | GATTCAGTCGTCACTCATGGTGATTTCTCACTTGATAACCTTATTTTTGACGAGGGGAAATTAATAGGTTGTATTGATGTTGGACGAGTCGGAATCGCAGACCGATACCAGGATCTTGCCATCCTATGGAACTGCCTCGGTGAGT |
| | TTTCTCCTTCATTACAGAAACGGCTTTTTCAAAAATATGGTATTGATAATCCTGATATGAATAAATTGCAGTTTCATTTGATGCTCGATGAGTTTTTCTAAGCGTATAATGGTCTAGAGCTAGCATATGGATCCATCGATTCCAT |
| | TATACGCCTGTCAGACCAAGTTTACTCATATATACTTTAGATTGATTTAAAACTTCATTTTTAATTTAAAAGGATCTAGGTGAAGATCCTTTTTGATAATCTCATGACCAAAATCCCTTAACGTGAGTTTTCGTTCCACTGAGCG |
| | TCAGACCCCGTAGAAAAGATCAAAGGATCTTCTTGAGATCCTTTTTTTCTGCGCGTAATCTGCTGCTTGCAAACAAAAAAACCACCGCTACCAGCGGTGGTTTGTTTGCCGGATCAAGAGCTACCAACTCTTTTTCCGAAGGTAA |
| | CTGGCTTCAGCAGAGCGCAGATACCAAATACTGTTCTTCTAGTGTAGCCGTAGTTAGGCCACCACTTCAAGAACTCTGTAGCACCGCCTACATACCTCGCTCTGCTAATCCTGTTACCAGTGGCTGCTGCCAGTGGCGATAAGTC |
| | GTGTCTTACCGGGTTGGACTCAAGACGATAGTTACCGGATAAGGCGCAGCGGTCGGGCTGAACGGGGGGTTCGTGCACACAGCCCAGCTTGGAGCGAACGACCTACACCGAACTGAGATACCTACAGCGTGAGCTATGAGAAAGC |
| | GCCACGCTTCCCGAAGGGAGAAAGGCGGACAGGTATCCGGTAAGCGGCAGGGTCGGAACAGGAGAGCGCACGAGGGAGCTTCCAGGGGGAAACGCCTGGTATCTTTATAGTCCTGTCGGGTTTCGCCACCTCTGACTTGAGCGTC |
| | GATTTTTGTGATGCTCGTCAGGGGGGCGGAGCCTATGGAAAAACGCCAGCAACGCGGCCTTTTTACGGTTCCTGGCCTTTTGCTGGCCTTTTGCTCACATGT |
| 31 | > ITR-to-ITR construct with coGAA GAA nucleic acid w/201 signal peptide |
| | CTGCGCGCTCGCTCGCTCACTGAGGCCGCCCGGGCAAAGCCCGGGCGTCGGGCGACCTTTGGTCGCCCGGCCTCAGTGAGCGAGCGAGCGCGCAGAGAGGGAGTGGCCAACTCCATCACTAGGGGTTCCTTGTAGTTAATGATTAA |
| | CCCGCCATGCTACTTATCTACGGCGCGCCACGCGTTCTAGGAGTTAATTTTTAAAAAGCAGTCAAAAGTCCAAGTGCCCTTGCGAGCATTTACTCTCTCTGTTTGCTCTGGTTAATAATCTCAGGAGCACAAACATTCCTTACTAG |
| | TTCTAGGAGTTAATTTTTAAAAAGCAGTCAAAAGTCCAAGTGCCCTTGCGAGCATTTACTCTCTCTGTTTGCTCTGGTTAATAATCTCAGGAGCACAAACATTCCTTACTAGTTCTAGAGCGGCCGCCAGTGTGCTGGAATTCGGC |
| | TTTTTTAGGGCTGGAAGCTACCTTTGACATCATTTCCTCTGCGAATGCATGTATAATTTCTACAGAACCTATTAGAAAGGATCACCCAGCCTCTGCTTTTGTACAACTTTCCCTTAAAAAACTGCCAATCCCACTGCTGTTTGGCC |
| | CAATAGTGAGAACTTTTTCCTGCTGCCTCTTGGTGCTTTTGCCTATGGCCCCTATTCTGCCTGCTGAAGACACTCTTGCCAGCATGGACTTAAACCCCTCCAGCTCTGACAATCCTCTTTCTCTTTTGTTTTACATGAAGGGTCTG |
| | GCAGCCAAAGCAATCACTCAAAGTTCAAACCTTATCATTTTTTGCTTTGTTCCTCTTGGCCTTGGTTTTGTACATCAGCTTTGAAAATACCATCCCAGGGTTAATGCTGGGGTTAATTTATAACTGAGAGTGCTCTAGTTCTGCAA |
| | TACAGGACATGCTATAAAAATGGAAAGATGTTGCTTTCTGAGAGATCAGGTGGATTCTTGGGCATTTGCTTAAGCTTGGTACCGCCACCATGGAGTTTGGGCTGAGCTGGGTCTTTCTGGTGGCCCTGCTGAAGGGAGTCCAGTGT |
| | GAGCAGCAGGGGGCCAGCAGGCCAGGCCCCAGGGATGCCCAGGCCCACCCTGGCAGACCCAGGGCTGTGCCCACCCAGTGTGATGTGCCTCCCAACAGCAGATTTGACTGTGCCCCTGATAAGGCCATTACTCAGGAACAGTGTGA |
| | GGCCAGGGGCTGCTGCTATATCCCTGCCAAGCAGGGCCTGCAGGGGGCCCAGATGGGGCAGCCCTGGTGCTTCTTCCCCCCCAGCTACCCCTCTTATAAGCTGGAGAATCTGAGCAGCTCTGAAATGGGCTACACTGCCACTCTGA |
| | CCAGGACTACCCCCACCTTCTTCCCCAAGGATATTCTGACTCTGAGGCTGGATGTGATGATGGAGACTGAGAACAGGCTGCACTTCACCATCAAGGACCCTGCCAACAGGAGGTATGAGGTGCCCCTGGAAACTCCCAGGGTGCAC |
| | TCTAGGGCCCCCAGCCCCCTGTATTCTGTGGAGTTCTCTGAGGAGCCTTTTGGGGTCATTGTCCACAGGCAGCTGGATGGCAGGGTGCTGCTGAATACTACTGTGGCCCCTCTGTTCTTTGCTGACCAGTTCCTGCAGCTGAGCAC |
| | CAGCCTGCCTTCTCAGTACATTACTGGCCTGGCTGAGCATCTGAGCCCCCTGATGCTGAGCACCTCTTGGACCAGAATCACCCTGTGGAACAGGGACCTGGCTCCCACTCCTGGGGCCAACCTGTATGGCAGCCACCCCTTCTACC |
| | TGGCCCTGGAGGATGGGGGCTCTGCCCATGGGGTGTTCCTGCTGAACAGCAATGCCATGGATGTGGTGCTGCAGCCCTCTCCTGCCCTGTCTTGGAGATCTACTGGGGGCATCCTGGATGTGTATATCTTCCTGGGGCCTGAGCCC |
| | AAGTCTGTGGTGCAGCAGTACCTGGATGTGGTGGGCTACCCCTTCATGCCCCCCTACTGGGGCCTGGGCTTCCACCTGTGCAGGTGGGGCTACTCTTCTACTGCTATCACCAGGCAGGTGGTGGAGAACATGACCAGGGCTCACTT |
| | CCCTCTGGATGTGCAGTGGAATGACCTGGACTACATGGACTCTAGGAGAGACTTCACTTTTAATAAGGATGGGTTCAGGGACTTTCCTGCCATGGTGCAGGAGCTGCATCAGGGGGGCAGGAGATATATGATGATTGTGGACCCTG |
| | CTATTTCTAGCTCTGGCCCTGCTGGCAGCTATAGGCCCTATGATGAGGGGCTGAGGAGGGGGGTGTTCATCACTAATGAGACTGGCCAGCCCCTGATTGGCAAGGTGTGGCCTGGCTCTACTGCCTTCCCTGATTTCACCAACCCC |
| | ACTGCCCTGGCCTGGTGGGAGGATATGGTGGCTGAGTTTCATGACCAGGTGCCCTTTGATGGCATGTGGATTGACATGAATGAGCCCAGCAACTTTATCAGGGGCTCTGAAGATGGCTGCCCCAACAATGAGCTGGAGAACCCCCC |
| | CTATGTGCCTGGGGTGGTGGGGGGCACCCTGCAGGCTGCCACCATCTGTGCCAGCAGCCACCAGTTCCTGAGCACCCACTATAACCTGCACAACCTGTATGGCCTGACTGAGGCCATTGCCTCTCACAGGGCCCTGGTGAAGGCTA |
| | GGGGGACTAGGCCCTTTGTGATCAGCAGGTCTACTTTTGCTGGCCATGGCAGGTATGCTGGGCACTGGACTGGGGATGTGTGGTCTAGCTGGGAGCAGCTGGCCAGCTCTGTGCCTGAGATCCTGCAGTTTAATCTGCTGGGGGTG |
| | CCCCTGGTGGGGGCTGATGTGTGTGGCTTCCTGGGCAATACCTCTGAGGAGCTGTGTGTGAGGTGGACTCAGCTGGGGGCTTTCTACCCCTTCATGAGAAACCACAACTCTCTGCTGAGCCTGCCCCAGGAGCCCTATTCTTTTTC |
| | TGAGCCTGCCCAGCAGGCTATGAGGAAGGCCCTGACTCTGAGGTATGCCCTGCTGCCCCACCTGTATACCCTGTTCCATCAGGCCCATGTGGCTGGGGAGACTGTGGCCAGACCTCTGTTCCTGGAGTTCCCCAAGGATAGCTCTA |
| | CTTGGACTGTGGACCACCAGCTGCTGTGGGGGGAGGCTCTGCTGATCACCCCTGTGCTGCAGGCTGGGAAGGCTGAGGTGACTGGCTATTTCCCCCTGGGCACCTGGTATGATCTGCAGACTGTGCCCATTGAGGCCCTGGGCTCT |
| | CTGCCCCCTCCCCCTGCTGCCCCCAGGGAGCCTGCCATCCACTCTGAGGGCCAGTGGGTGACCCTGCCTGCCCCTCTGGACACTATCAATGTGCACCTGAGGGCTGGCTACATCATCCCCCTGCAGGGCCCTGGCCTGACTACCAC |
| | TGAGTCTAGGCAGCAGCCCATGGCCCTGGCTGTGGCTCTGACCAAGGGGGGGGAGGCCAGGGGGGAGCTGTTCTGGGATGATGGGGAGTCTCTGGAGGTGCTGGAGAGGGGGGCCTACACCCAGGTGATCTTCCTGGCTAGGAATA |
| | ACACCATTGTCAATGAGCTGGTGAGGGTGACCTCTGAGGGGGCTGGCCTGCAGCTGCAGAAGGTGACTGTGCTGGGGGTGGCTACTGCCCCCCAGCAGGTGCTGAGCAATGGGGTGCCTGTGAGCAACTTCACCTATAGCCCTGAC |
| | ACCAAGGTGCTGGACATCTGTGTGAGCCTGCTGATGGGGGAGCAGTTCCTGGTGAGCTGGTGCTAATTTAAATCTCGAGCCGGGCGGAGTGTGTTAGTCTCTCCAGAGGGAGGCTGGTTCCCCAGGGAAGCAGAGCCTGTGTGCGG |
| | GCAGCAGCTGTGTGCGGGCCTGGGGGTTGTTAAGTGCAATTATTTTTAATAAAAGGGGCATTTGGAAAAAAAAAAAAAAGGTAGCAGTCGACAGATGAATTCTGCAGATCTGTGGCTTCTAGCTGCCCGGGTGGCATCCCTGTGAC |
| | CCCTCCCCAGTGCCTCTCCTGGCCCTGGAAGTTGCCACTCCAGTGCCCACCAGCCTTGTCCTAATAAAATTAAGTTGCATCATTTTGTCTGACTAGGTGTCCTTCTATAATATTATGGGGTGGAGGGGGGTGGTATGGAGCAAGGG |
| | GCAAGTTGGGAAGACAACCTGTAGGGCCTGCGGGGTCTATTGGGAACCAAGCTGGAGTGCAGTGGCACAATCTTGGCTCACTGCAATCTCCGCCTCCTGGGTTCAAGCGATTCTCCTGCCTCAGCCTCCCGAGTTGTTGGGATTCC |
| | AGGCATGCATGACCAGGCTCAGCTAATTTTTGTTTTTTTGGTAGAGACGGGGTTTCACCATATTGGCCAGGCTGGTCTCCAACTCCTAATCTCAGGTGATCTACCCACCTTGGCCTCCCAAATTGCTGGGATTACAGGCGTGAACC |
| | ACTGCTCCCTTCCCTGTCCTTCTGATTTTAAAAAAAAGAGAAATGTTCTGGCACCTGCACTTGCACTGGGGACAGCCTATTTTGCTAGTTTGTTTTGTTTCGTTTTGTTTTGATGGAGAGCGTATGTTGTTTAAACGCGGCCGCGT |
| | AGATAAGTAGCATGGCGGGTTAATCATTAACTACAAGGAACCCCTAGTGATGGAGTTGGCCACTCCCTCTCTGCGCGCTCGCTCGCTCACTGAGGCCGGGCGACCAAAGGTCGCCCGACGCCCGGGCTTTGCCCGGGCGGCCTCAG |
| | TGAGCGAGCGAGCGCGCAG |
| 32 | > ITR-to-ITR construct with coGAA GAA nucleic acid w/wtIL signal peptide |
| | CTGCGCGCTCGCTCGCTCACTGAGGCCGCCCGGGCAAAGCCCGGGCGTCGGGCGACCTTTGGTCGCCCGGCCTCAGTGAGCGAGCGAGCGCGCAGAGAGGGAGTGGCCAACTCCATCACTAGGGGTTCCTTGTAGTTAATGATTAA |
| | CCCGCCATGCTACTTATCTACGGCGCGCCACGCGTTCTAGGAGTTAATTTTTAAAAAGCAGTCAAAAGTCCAAGTGCCCTTGCGAGCATTTACTCTCTCTGTTTGCTCTGGTTAATAATCTCAGGAGCACAAACATTCCTTACTAG |
| | TTCTAGGAGTTAATTTTTAAAAAGCAGTCAAAAGTCCAAGTGCCCTTGCGAGCATTTACTCTCTCTGTTTGCTCTGGTTAATAATCTCAGGAGCACAAACATTCCTTACTAGTTCTAGAGCGGCCGCCAGTGTGCTGGAATTCGGC |
| | TTTTTTAGGGCTGGAAGCTACCTTTGACATCATTTCCTCTGCGAATGCATGTATAATTTCTACAGAACCTATTAGAAAGGATCACCCAGCCTCTGCTTTTGTACAACTTTCCCTTAAAAAACTGCCAATCCCACTGCTGTTTGGCC |
| | CAATAGTGAGAACTTTTTCCTGCTGCCTCTTGGTGCTTTTGCCTATGGCCCCTATTCTGCCTGCTGAAGACACTCTTGCCAGCATGGACTTAAACCCCTCCAGCTCTGACAATCCTCTTTCTCTTTTGTTTTACATGAAGGGTCTG |
| | GCAGCCAAAGCAATCACTCAAAGTTCAAACCTTATCATTTTTTGCTTTGTTCCTCTTGGCCTTGGTTTTGTACATCAGCTTTGAAAATACCATCCCAGGGTTAATGCTGGGGTTAATTTATAACTGAGAGTGCTCTAGTTCTGCAA |
| | TACAGGACATGCTATAAAAATGGAAAGATGTTGCTTTCTGAGAGATCAGGTGGATTCTTGGGCATTTGCTTAAGCTTGGTACCGCCACCATGTACAGAATGCAGCTGCTGTCCTGCATTGCACTGTCACTGGCACTGGTTACCAAC |
| | TCACAGCAGGGGGCCAGCAGGCCAGGCCCCAGGGATGCCCAGGCCCACCCTGGCAGACCCAGGGCTGTGCCCACCCAGTGTGATGTGCCTCCCAACAGCAGATTTGACTGTGCCCCTGATAAGGCCATTACTCAGGAACAGTGTGA |
| | GGCCAGGGGCTGCTGCTATATCCCTGCCAAGCAGGGCCTGCAGGGGGCCCAGATGGGGCAGCCCTGGTGCTTCTTCCCCCCCAGCTACCCCTCTTATAAGCTGGAGAATCTGAGCAGCTCTGAAATGGGCTACACTGCCACTCTGA |
| | CCAGGACTACCCCCACCTTCTTCCCCAAGGATATTCTGACTCTGAGGCTGGATGTGATGATGGAGACTGAGAACAGGCTGCACTTCACCATCAAGGACCCTGCCAACAGGAGGTATGAGGTGCCCCTGGAAACTCCCAGGGTGCAC |
| | TCTAGGGCCCCCAGCCCCCTGTATTCTGTGGAGTTCTCTGAGGAGCCTTTTGGGGTCATTGTCCACAGGCAGCTGGATGGCAGGGTGCTGCTGAATACTACTGTGGCCCCTCTGTTCTTTGCTGACCAGTTCCTGCAGCTGAGCAC |
| | CAGCCTGCCTTCTCAGTACATTACTGGCCTGGCTGAGCATCTGAGCCCCCTGATGCTGAGCACCTCTTGGACCAGAATCACCCTGTGGAACAGGGACCTGGCTCCCACTCCTGGGGCCAACCTGTATGGCAGCCACCCCTTCTACC |
| | TGGCCCTGGAGGATGGGGGCTCTGCCCATGGGGTGTTCCTGCTGAACAGCAATGCCATGGATGTGGTGCTGCAGCCCTCTCCTGCCCTGTCTTGGAGATCTACTGGGGGCATCCTGGATGTGTATATCTTCCTGGGGCCTGAGCCC |
| | AAGTCTGTGGTGCAGCAGTACCTGGATGTGGTGGGCTACCCCTTCATGCCCCCCTACTGGGGCCTGGGCTTCCACCTGTGCAGGTGGGGCTACTCTTCTACTGCTATCACCAGGCAGGTGGTGGAGAACATGACCAGGGCTCACTT |
| | CCCTCTGGATGTGCAGTGGAATGACCTGGACTACATGGACTCTAGGAGAGACTTCACTTTTAATAAGGATGGGTTCAGGGACTTTCCTGCCATGGTGCAGGAGCTGCATCAGGGGGGCAGGAGATATATGATGATTGTGGACCCTG |
| | CTATTTCTAGCTCTGGCCCTGCTGGCAGCTATAGGCCCTATGATGAGGGGCTGAGGAGGGGGGTGTTCATCACTAATGAGACTGGCCAGCCCCTGATTGGCAAGGTGTGGCCTGGCTCTACTGCCTTCCCTGATTTCACCAACCCC |
| | ACTGCCCTGGCCTGGTGGGAGGATATGGTGGCTGAGTTTCATGACCAGGTGCCCTTTGATGGCATGTGGATTGACATGAATGAGCCCAGCAACTTTATCAGGGGCTCTGAAGATGGCTGCCCCAACAATGAGCTGGAGAACCCCCC |
| | CTATGTGCCTGGGGTGGTGGGGGGCACCCTGCAGGCTGCCACCATCTGTGCCAGCAGCCACCAGTTCCTGAGCACCCACTATAACCTGCACAACCTGTATGGCCTGACTGAGGCCATTGCCTCTCACAGGGCCCTGGTGAAGGCTA |
| | GGGGGACTAGGCCCTTTGTGATCAGCAGGTCTACTTTTGCTGGCCATGGCAGGTATGCTGGGCACTGGACTGGGGATGTGTGGTCTAGCTGGGAGCAGCTGGCCAGCTCTGTGCCTGAGATCCTGCAGTTTAATCTGCTGGGGGTG |
| | CCCCTGGTGGGGGCTGATGTGTGTGGCTTCCTGGGCAATACCTCTGAGGAGCTGTGTGTGAGGTGGACTCAGCTGGGGGCTTTCTACCCCTTCATGAGAAACCACAACTCTCTGCTGAGCCTGCCCCAGGAGCCCTATTCTTTTTC |
| | TGAGCCTGCCCAGCAGGCTATGAGGAAGGCCCTGACTCTGAGGTATGCCCTGCTGCCCCACCTGTATACCCTGTTCCATCAGGCCCATGTGGCTGGGGAGACTGTGGCCAGACCTCTGTTCCTGGAGTTCCCCAAGGATAGCTCTA |
| | CTTGGACTGTGGACCACCAGCTGCTGTGGGGGGAGGCTCTGCTGATCACCCCTGTGCTGCAGGCTGGGAAGGCTGAGGTGACTGGCTATTTCCCCCTGGGCACCTGGTATGATCTGCAGACTGTGCCCATTGAGGCCCTGGGCTCT |
| | CTGCCCCCTCCCCCTGCTGCCCCCAGGGAGCCTGCCATCCACTCTGAGGGCCAGTGGGTGACCCTGCCTGCCCCTCTGGACACTATCAATGTGCACCTGAGGGCTGGCTACATCATCCCCCTGCAGGGCCCTGGCCTGACTACCAC |
| | TGAGTCTAGGCAGCAGCCCATGGCCCTGGCTGTGGCTCTGACCAAGGGGGGGGAGGCCAGGGGGGAGCTGTTCTGGGATGATGGGGAGTCTCTGGAGGTGCTGGAGAGGGGGGCCTACACCCAGGTGATCTTCCTGGCTAGGAATA |
| | ACACCATTGTCAATGAGCTGGTGAGGGTGACCTCTGAGGGGGCTGGCCTGCAGCTGCAGAAGGTGACTGTGCTGGGGGTGGCTACTGCCCCCCAGCAGGTGCTGAGCAATGGGGTGCCTGTGAGCAACTTCACCTATAGCCCTGAC |
| | ACCAAGGTGCTGGACATCTGTGTGAGCCTGCTGATGGGGGAGCAGTTCCTGGTGAGCTGGTGCTAATTTAAATCTCGAGCCGGGCGGAGTGTGTTAGTCTCTCCAGAGGGAGGCTGGTTCCCCAGGGAAGCAGAGCCTGTGTGCGG |
| | GCAGCAGCTGTGTGCGGGCCTGGGGGTTGTTAAGTGCAATTATTTTTAATAAAAGGGGCATTTGGAAAAAAAAAAAAAAGGTAGCAGTCGACAGATGAATTCTGCAGATCTGTGGCTTCTAGCTGCCCGGGTGGCATCCCTGTGAC |
| | CCCTCCCCAGTGCCTCTCCTGGCCCTGGAAGTTGCCACTCCAGTGCCCACCAGCCTTGTCCTAATAAAATTAAGTTGCATCATTTTGTCTGACTAGGTGTCCTTCTATAATATTATGGGGTGGAGGGGGGTGGTATGGAGCAAGGG |
| | GCAAGTTGGGAAGACAACCTGTAGGGCCTGCGGGGTCTATTGGGAACCAAGCTGGAGTGCAGTGGCACAATCTTGGCTCACTGCAATCTCCGCCTCCTGGGTTCAAGCGATTCTCCTGCCTCAGCCTCCCGAGTTGTTGGGATTCC |
| | AGGCATGCATGACCAGGCTCAGCTAATTTTTGTTTTTTTGGTAGAGACGGGGTTTCACCATATTGGCCAGGCTGGTCTCCAACTCCTAATCTCAGGTGATCTACCCACCTTGGCCTCCCAAATTGCTGGGATTACAGGCGTGAACC |
| | ACTGCTCCCTTCCCTGTCCTTCTGATTTTAAAAAAAAGAGAAATGTTCTGGCACCTGCACTTGCACTGGGGACAGCCTATTTTGCTAGTTTGTTTTGTTTCGTTTTGTTTTGATGGAGAGCGTATGTTGTTTAAACGCGGCCGCGT |
| | AGATAAGTAGCATGGCGGGTTAATCATTAACTACAAGGAACCCCTAGTGATGGAGTTGGCCACTCCCTCTCTGCGCGCTCGCTCGCTCACTGAGGCCGGGCGACCAAAGGTCGCCCGACGCCCGGGCTTTGCCCGGGCGGCCTCAG |
| | TGAGCGAGCGAGCGCGCAG |
| 33 | > ITR-to-ITR construct with coGAA GAA nucleic acid w/ILmut signal peptide |
| | CTGCGCGCTCGCTCGCTCACTGAGGCCGCCCGGGCAAAGCCCGGGCGTCGGGCGACCTTTGGTCGCCCGGCCTCAGTGAGCGAGCGAGCGCGCAGAGAGGGAGTGGCCAACTCCATCACTAGGGGTTCCTTGTAGTTAATGATTAA |
| | CCCGCCATGCTACTTATCTACGGCGCGCCACGCGTTCTAGGAGTTAATTTTTAAAAAGCAGTCAAAAGTCCAAGTGCCCTTGCGAGCATTTACTCTCTCTGTTTGCTCTGGTTAATAATCTCAGGAGCACAAACATTCCTTACTAG |
| | TTCTAGGAGTTAATTTTTAAAAAGCAGTCAAAAGTCCAAGTGCCCTTGCGAGCATTTACTCTCTCTGTTTGCTCTGGTTAATAATCTCAGGAGCACAAACATTCCTTACTAGTTCTAGAGCGGCCGCCAGTGTGCTGGAATTCGGC |
| | TTTTTTAGGGCTGGAAGCTACCTTTGACATCATTTCCTCTGCGAATGCATGTATAATTTCTACAGAACCTATTAGAAAGGATCACCCAGCCTCTGCTTTTGTACAACTTTCCCTTAAAAAACTGCCAATCCCACTGCTGTTTGGCC |
| | CAATAGTGAGAACTTTTTCCTGCTGCCTCTTGGTGCTTTTGCCTATGGCCCCTATTCTGCCTGCTGAAGACACTCTTGCCAGCATGGACTTAAACCCCTCCAGCTCTGACAATCCTCTTTCTCTTTTGTTTTACATGAAGGGTCTG |
| | GCAGCCAAAGCAATCACTCAAAGTTCAAACCTTATCATTTTTTGCTTTGTTCCTCTTGGCCTTGGTTTTGTACATCAGCTTTGAAAATACCATCCCAGGGTTAATGCTGGGGTTAATTTATAACTGAGAGTGCTCTAGTTCTGCAA |
| | TACAGGACATGCTATAAAAATGGAAAGATGTTGCTTTCTGAGAGATCAGGTGGATTCTTGGGCATTTGCTTAAGCTTGGTACCGCCACCATGTACAGAATGCAGCTGCTGCTGCTGATTGCACTGTCACTGGCACTGGTTACCAAC |
| | TCACAGCAGGGGGCCAGCAGGCCAGGCCCCAGGGATGCCCAGGCCCACCCTGGCAGACCCAGGGCTGTGCCCACCCAGTGTGATGTGCCTCCCAACAGCAGATTTGACTGTGCCCCTGATAAGGCCATTACTCAGGAACAGTGTGA |
| | GGCCAGGGGCTGCTGCTATATCCCTGCCAAGCAGGGCCTGCAGGGGGCCCAGATGGGGCAGCCCTGGTGCTTCTTCCCCCCCAGCTACCCCTCTTATAAGCTGGAGAATCTGAGCAGCTCTGAAATGGGCTACACTGCCACTCTGA |
| | CCAGGACTACCCCCACCTTCTTCCCCAAGGATATTCTGACTCTGAGGCTGGATGTGATGATGGAGACTGAGAACAGGCTGCACTTCACCATCAAGGACCCTGCCAACAGGAGGTATGAGGTGCCCCTGGAAACTCCCAGGGTGCAC |
| | TCTAGGGCCCCCAGCCCCCTGTATTCTGTGGAGTTCTCTGAGGAGCCTTTTGGGGTCATTGTCCACAGGCAGCTGGATGGCAGGGTGCTGCTGAATACTACTGTGGCCCCTCTGTTCTTTGCTGACCAGTTCCTGCAGCTGAGCAC |
| | CAGCCTGCCTTCTCAGTACATTACTGGCCTGGCTGAGCATCTGAGCCCCCTGATGCTGAGCACCTCTTGGACCAGAATCACCCTGTGGAACAGGGACCTGGCTCCCACTCCTGGGGCCAACCTGTATGGCAGCCACCCCTTCTACC |
| | TGGCCCTGGAGGATGGGGGCTCTGCCCATGGGGTGTTCCTGCTGAACAGCAATGCCATGGATGTGGTGCTGCAGCCCTCTCCTGCCCTGTCTTGGAGATCTACTGGGGGCATCCTGGATGTGTATATCTTCCTGGGGCCTGAGCCC |
| | AAGTCTGTGGTGCAGCAGTACCTGGATGTGGTGGGCTACCCCTTCATGCCCCCCTACTGGGGCCTGGGCTTCCACCTGTGCAGGTGGGGCTACTCTTCTACTGCTATCACCAGGCAGGTGGTGGAGAACATGACCAGGGCTCACTT |
| | CCCTCTGGATGTGCAGTGGAATGACCTGGACTACATGGACTCTAGGAGAGACTTCACTTTTAATAAGGATGGGTTCAGGGACTTTCCTGCCATGGTGCAGGAGCTGCATCAGGGGGGCAGGAGATATATGATGATTGTGGACCCTG |
| | CTATTTCTAGCTCTGGCCCTGCTGGCAGCTATAGGCCCTATGATGAGGGGCTGAGGAGGGGGGTGTTCATCACTAATGAGACTGGCCAGCCCCTGATTGGCAAGGTGTGGCCTGGCTCTACTGCCTTCCCTGATTTCACCAACCCC |
| | ACTGCCCTGGCCTGGTGGGAGGATATGGTGGCTGAGTTTCATGACCAGGTGCCCTTTGATGGCATGTGGATTGACATGAATGAGCCCAGCAACTTTATCAGGGGCTCTGAAGATGGCTGCCCCAACAATGAGCTGGAGAACCCCCC |
| | CTATGTGCCTGGGGTGGTGGGGGGCACCCTGCAGGCTGCCACCATCTGTGCCAGCAGCCACCAGTTCCTGAGCACCCACTATAACCTGCACAACCTGTATGGCCTGACTGAGGCCATTGCCTCTCACAGGGCCCTGGTGAAGGCTA |
| | GGGGGACTAGGCCCTTTGTGATCAGCAGGTCTACTTTTGCTGGCCATGGCAGGTATGCTGGGCACTGGACTGGGGATGTGTGGTCTAGCTGGGAGCAGCTGGCCAGCTCTGTGCCTGAGATCCTGCAGTTTAATCTGCTGGGGGTG |
| | CCCCTGGTGGGGGCTGATGTGTGTGGCTTCCTGGGCAATACCTCTGAGGAGCTGTGTGTGAGGTGGACTCAGCTGGGGGCTTTCTACCCCTTCATGAGAAACCACAACTCTCTGCTGAGCCTGCCCCAGGAGCCCTATTCTTTTTC |
| | TGAGCCTGCCCAGCAGGCTATGAGGAAGGCCCTGACTCTGAGGTATGCCCTGCTGCCCCACCTGTATACCCTGTTCCATCAGGCCCATGTGGCTGGGGAGACTGTGGCCAGACCTCTGTTCCTGGAGTTCCCCAAGGATAGCTCTA |
| | CTTGGACTGTGGACCACCAGCTGCTGTGGGGGGAGGCTCTGCTGATCACCCCTGTGCTGCAGGCTGGGAAGGCTGAGGTGACTGGCTATTTCCCCCTGGGCACCTGGTATGATCTGCAGACTGTGCCCATTGAGGCCCTGGGCTCT |
| | CTGCCCCCTCCCCCTGCTGCCCCCAGGGAGCCTGCCATCCACTCTGAGGGCCAGTGGGTGACCCTGCCTGCCCCTCTGGACACTATCAATGTGCACCTGAGGGCTGGCTACATCATCCCCCTGCAGGGCCCTGGCCTGACTACCAC |
| | TGAGTCTAGGCAGCAGCCCATGGCCCTGGCTGTGGCTCTGACCAAGGGGGGGGAGGCCAGGGGGGAGCTGTTCTGGGATGATGGGGAGTCTCTGGAGGTGCTGGAGAGGGGGGCCTACACCCAGGTGATCTTCCTGGCTAGGAATA |
| | ACACCATTGTCAATGAGCTGGTGAGGGTGACCTCTGAGGGGGCTGGCCTGCAGCTGCAGAAGGTGACTGTGCTGGGGGTGGCTACTGCCCCCCAGCAGGTGCTGAGCAATGGGGTGCCTGTGAGCAACTTCACCTATAGCCCTGAC |
| | ACCAAGGTGCTGGACATCTGTGTGAGCCTGCTGATGGGGGAGCAGTTCCTGGTGAGCTGGTGCTAATTTAAATCTCGAGCCGGGCGGAGTGTGTTAGTCTCTCCAGAGGGAGGCTGGTTCCCCAGGGAAGCAGAGCCTGTGTGCGG |
| | GCAGCAGCTGTGTGCGGGCCTGGGGGTTGTTAAGTGCAATTATTTTTAATAAAAGGGGCATTTGGAAAAAAAAAAAAAAGGTAGCAGTCGACAGATGAATTCTGCAGATCTGTGGCTTCTAGCTGCCCGGGTGGCATCCCTGTGAC |
| | CCCTCCCCAGTGCCTCTCCTGGCCCTGGAAGTTGCCACTCCAGTGCCCACCAGCCTTGTCCTAATAAAATTAAGTTGCATCATTTTGTCTGACTAGGTGTCCTTCTATAATATTATGGGGTGGAGGGGGGTGGTATGGAGCAAGGG |
| | GCAAGTTGGGAAGACAACCTGTAGGGCCTGCGGGGTCTATTGGGAACCAAGCTGGAGTGCAGTGGCACAATCTTGGCTCACTGCAATCTCCGCCTCCTGGGTTCAAGCGATTCTCCTGCCTCAGCCTCCCGAGTTGTTGGGATTCC |
| | AGGCATGCATGACCAGGCTCAGCTAATTTTTGTTTTTTTGGTAGAGACGGGGTTTCACCATATTGGCCAGGCTGGTCTCCAACTCCTAATCTCAGGTGATCTACCCACCTTGGCCTCCCAAATTGCTGGGATTACAGGCGTGAACC |
| | ACTGCTCCCTTCCCTGTCCTTCTGATTTTAAAAAAAAGAGAAATGTTCTGGCACCTGCACTTGCACTGGGGACAGCCTATTTTGCTAGTTTGTTTTGTTTCGTTTTGTTTTGATGGAGAGCGTATGTTGTTTAAACGCGGCCGCGT |
| | AGATAAGTAGCATGGCGGGTTAATCATTAACTACAAGGAACCCCTAGTGATGGAGTTGGCCACTCCCTCTCTGCGCGCTCGCTCGCTCACTGAGGCCGGGCGACCAAAGGTCGCCCGACGCCCGGGCTTTGCCCGGGCGGCCTCAG |
| | TGAGCGAGCGAGCGCGCAG |
| 34 | > ITR-to-ITR construct with coGAA GAA nucleic acid w/A2 signal peptide |
| | CTGCGCGCTCGCTCGCTCACTGAGGCCGCCCGGGCAAAGCCCGGGCGTCGGGCGACCTTTGGTCGCCCGGCCTCAGTGAGCGAGCGAGCGCGCAGAGAGGGAGTGGCCAACTCCATCACTAGGGGTTCCTTGTAGTTAATGATTAA |
| | CCCGCCATGCTACTTATCTACGGCGCGCCACGCGTTCTAGGAGTTAATTTTTAAAAAGCAGTCAAAAGTCCAAGTGCCCTTGCGAGCATTTACTCTCTCTGTTTGCTCTGGTTAATAATCTCAGGAGCACAAACATTCCTTACTAG |
| | TTCTAGGAGTTAATTTTTAAAAAGCAGTCAAAAGTCCAAGTGCCCTTGCGAGCATTTACTCTCTCTGTTTGCTCTGGTTAATAATCTCAGGAGCACAAACATTCCTTACTAGTTCTAGAGCGGCCGCCAGTGTGCTGGAATTCGGC |
| | TTTTTTAGGGCTGGAAGCTACCTTTGACATCATTTCCTCTGCGAATGCATGTATAATTTCTACAGAACCTATTAGAAAGGATCACCCAGCCTCTGCTTTTGTACAACTTTCCCTTAAAAAACTGCCAATCCCACTGCTGTTTGGCC |
| | CAATAGTGAGAACTTTTTCCTGCTGCCTCTTGGTGCTTTTGCCTATGGCCCCTATTCTGCCTGCTGAAGACACTCTTGCCAGCATGGACTTAAACCCCTCCAGCTCTGACAATCCTCTTTCTCTTTTGTTTTACATGAAGGGTCTG |
| | GCAGCCAAAGCAATCACTCAAAGTTCAAACCTTATCATTTTTTGCTTTGTTCCTCTTGGCCTTGGTTTTGTACATCAGCTTTGAAAATACCATCCCAGGGTTAATGCTGGGGTTAATTTATAACTGAGAGTGCTCTAGTTCTGCAA |
| | TACAGGACATGCTATAAAAATGGAAAGATGTTGCTTTCTGAGAGATCAGGTGGATTCTTGGGCATTTGCTTAAGCTTGGTACCGCCACCATGGGGAAGAACAAACTCCTTCATCCAAGTCTGGTTCTTCTCCTCTTGGTCCTCCTG |
| | CCCACAGATGCCCAGCAGGGGGCCAGCAGGCCAGGCCCCAGGGATGCCCAGGCCCACCCTGGCAGACCCAGGGCTGTGCCCACCCAGTGTGATGTGCCTCCCAACAGCAGATTTGACTGTGCCCCTGATAAGGCCATTACTCAGGA |
| | ACAGTGTGAGGCCAGGGGCTGCTGCTATATCCCTGCCAAGCAGGGCCTGCAGGGGGCCCAGATGGGGCAGCCCTGGTGCTTCTTCCCCCCCAGCTACCCCTCTTATAAGCTGGAGAATCTGAGCAGCTCTGAAATGGGCTACACTG |
| | CCACTCTGACCAGGACTACCCCCACCTTCTTCCCCAAGGATATTCTGACTCTGAGGCTGGATGTGATGATGGAGACTGAGAACAGGCTGCACTTCACCATCAAGGACCCTGCCAACAGGAGGTATGAGGTGCCCCTGGAAACTCCC |
| | AGGGTGCACTCTAGGGCCCCCAGCCCCCTGTATTCTGTGGAGTTCTCTGAGGAGCCTTTTGGGGTCATTGTCCACAGGCAGCTGGATGGCAGGGTGCTGCTGAATACTACTGTGGCCCCTCTGTTCTTTGCTGACCAGTTCCTGCA |
| | GCTGAGCACCAGCCTGCCTTCTCAGTACATTACTGGCCTGGCTGAGCATCTGAGCCCCCTGATGCTGAGCACCTCTTGGACCAGAATCACCCTGTGGAACAGGGACCTGGCTCCCACTCCTGGGGCCAACCTGTATGGCAGCCACC |
| | CCTTCTACCTGGCCCTGGAGGATGGGGGCTCTGCCCATGGGGTGTTCCTGCTGAACAGCAATGCCATGGATGTGGTGCTGCAGCCCTCTCCTGCCCTGTCTTGGAGATCTACTGGGGGCATCCTGGATGTGTATATCTTCCTGGGG |
| | CCTGAGCCCAAGTCTGTGGTGCAGCAGTACCTGGATGTGGTGGGCTACCCCTTCATGCCCCCCTACTGGGGCCTGGGCTTCCACCTGTGCAGGTGGGGCTACTCTTCTACTGCTATCACCAGGCAGGTGGTGGAGAACATGACCAG |
| | GGCTCACTTCCCTCTGGATGTGCAGTGGAATGACCTGGACTACATGGACTCTAGGAGAGACTTCACTTTTAATAAGGATGGGTTCAGGGACTTTCCTGCCATGGTGCAGGAGCTGCATCAGGGGGGCAGGAGATATATGATGATTG |
| | TGGACCCTGCTATTTCTAGCTCTGGCCCTGCTGGCAGCTATAGGCCCTATGATGAGGGGCTGAGGAGGGGGGTGTTCATCACTAATGAGACTGGCCAGCCCCTGATTGGCAAGGTGTGGCCTGGCTCTACTGCCTTCCCTGATTTC |
| | ACCAACCCCACTGCCCTGGCCTGGTGGGAGGATATGGTGGCTGAGTTTCATGACCAGGTGCCCTTTGATGGCATGTGGATTGACATGAATGAGCCCAGCAACTTTATCAGGGGCTCTGAAGATGGCTGCCCCAACAATGAGCTGGA |
| | GAACCCCCCCTATGTGCCTGGGGTGGTGGGGGGCACCCTGCAGGCTGCCACCATCTGTGCCAGCAGCCACCAGTTCCTGAGCACCCACTATAACCTGCACAACCTGTATGGCCTGACTGAGGCCATTGCCTCTCACAGGGCCCTGG |
| | TGAAGGCTAGGGGGACTAGGCCCTTTGTGATCAGCAGGTCTACTTTTGCTGGCCATGGCAGGTATGCTGGGCACTGGACTGGGGATGTGTGGTCTAGCTGGGAGCAGCTGGCCAGCTCTGTGCCTGAGATCCTGCAGTTTAATCTG |
| | CTGGGGGTGCCCCTGGTGGGGGCTGATGTGTGTGGCTTCCTGGGCAATACCTCTGAGGAGCTGTGTGTGAGGTGGACTCAGCTGGGGGCTTTCTACCCCTTCATGAGAAACCACAACTCTCTGCTGAGCCTGCCCCAGGAGCCCTA |
| | TTCTTTTTCTGAGCCTGCCCAGCAGGCTATGAGGAAGGCCCTGACTCTGAGGTATGCCCTGCTGCCCCACCTGTATACCCTGTTCCATCAGGCCCATGTGGCTGGGGAGACTGTGGCCAGACCTCTGTTCCTGGAGTTCCCCAAGG |
| | ATAGCTCTACTTGGACTGTGGACCACCAGCTGCTGTGGGGGGAGGCTCTGCTGATCACCCCTGTGCTGCAGGCTGGGAAGGCTGAGGTGACTGGCTATTTCCCCCTGGGCACCTGGTATGATCTGCAGACTGTGCCCATTGAGGCC |
| | CTGGGCTCTCTGCCCCCTCCCCCTGCTGCCCCCAGGGAGCCTGCCATCCACTCTGAGGGCCAGTGGGTGACCCTGCCTGCCCCTCTGGACACTATCAATGTGCACCTGAGGGCTGGCTACATCATCCCCCTGCAGGGCCCTGGCCT |
| | GACTACCACTGAGTCTAGGCAGCAGCCCATGGCCCTGGCTGTGGCTCTGACCAAGGGGGGGGAGGCCAGGGGGGAGCTGTTCTGGGATGATGGGGAGTCTCTGGAGGTGCTGGAGAGGGGGGCCTACACCCAGGTGATCTTCCTGG |
| | CTAGGAATAACACCATTGTCAATGAGCTGGTGAGGGTGACCTCTGAGGGGGCTGGCCTGCAGCTGCAGAAGGTGACTGTGCTGGGGGTGGCTACTGCCCCCCAGCAGGTGCTGAGCAATGGGGTGCCTGTGAGCAACTTCACCTAT |
| | AGCCCTGACACCAAGGTGCTGGACATCTGTGTGAGCCTGCTGATGGGGGAGCAGTTCCTGGTGAGCTGGTGCTAATTTAAATCTCGAGCCGGGCGGAGTGTGTTAGTCTCTCCAGAGGGAGGCTGGTTCCCCAGGGAAGCAGAGCC |
| | TGTGTGCGGGCAGCAGCTGTGTGCGGGCCTGGGGGTTGTTAAGTGCAATTATTTTTAATAAAAGGGGCATTTGGAAAAAAAAAAAAAAGGTAGCAGTCGACAGATGAATTCTGCAGATCTGTGGCTTCTAGCTGCCCGGGTGGCAT |
| | CCCTGTGACCCCTCCCCAGTGCCTCTCCTGGCCCTGGAAGTTGCCACTCCAGTGCCCACCAGCCTTGTCCTAATAAAATTAAGTTGCATCATTTTGTCTGACTAGGTGTCCTTCTATAATATTATGGGGTGGAGGGGGGTGGTATG |
| | GAGCAAGGGGCAAGTTGGGAAGACAACCTGTAGGGCCTGCGGGGTCTATTGGGAACCAAGCTGGAGTGCAGTGGCACAATCTTGGCTCACTGCAATCTCCGCCTCCTGGGTTCAAGCGATTCTCCTGCCTCAGCCTCCCGAGTTGT |
| | TGGGATTCCAGGCATGCATGACCAGGCTCAGCTAATTTTTGTTTTTTTGGTAGAGACGGGGTTTCACCATATTGGCCAGGCTGGTCTCCAACTCCTAATCTCAGGTGATCTACCCACCTTGGCCTCCCAAATTGCTGGGATTACAG |
| | GCGTGAACCACTGCTCCCTTCCCTGTCCTTCTGATTTTAAAAAAAAGAGAAATGTTCTGGCACCTGCACTTGCACTGGGGACAGCCTATTTTGCTAGTTTGTTTTGTTTCGTTTTGTTTTGATGGAGAGCGTATGTTGTTTAAACG |
| | CGGCCGCGTAGATAAGTAGCATGGCGGGTTAATCATTAACTACAAGGAACCCCTAGTGATGGAGTTGGCCACTCCCTCTCTGCGCGCTCGCTCGCTCACTGAGGCCGGGCGACCAAAGGTCGCCCGACGCCCGGGCTTTGCCCGGG |
| | CGGCCTCAGTGAGCGAGCGAGCGCGCAG |
| 35 | > ITR-to-ITR construct with coGAA GAA nucleic acid w/PXP signal peptide |
| | CTGCGCGCTCGCTCGCTCACTGAGGCCGCCCGGGCAAAGCCCGGGCGTCGGGCGACCTTTGGTCGCCCGGCCTCAGTGAGCGAGCGAGCGCGCAGAGAGGGAGTGGCCAACTCCATCACTAGGGGTTCCTTGTAGTTAATGATTAA |
| | CCCGCCATGCTACTTATCTACGGCGCGCCACGCGTTCTAGGAGTTAATTTTTAAAAAGCAGTCAAAAGTCCAAGTGCCCTTGCGAGCATTTACTCTCTCTGTTTGCTCTGGTTAATAATCTCAGGAGCACAAACATTCCTTACTAG |
| | TTCTAGGAGTTAATTTTTAAAAAGCAGTCAAAAGTCCAAGTGCCCTTGCGAGCATTTACTCTCTCTGTTTGCTCTGGTTAATAATCTCAGGAGCACAAACATTCCTTACTAGTTCTAGAGCGGCCGCCAGTGTGCTGGAATTCGGC |
| | TTTTTTAGGGCTGGAAGCTACCTTTGACATCATTTCCTCTGCGAATGCATGTATAATTTCTACAGAACCTATTAGAAAGGATCACCCAGCCTCTGCTTTTGTACAACTTTCCCTTAAAAAACTGCCAATCCCACTGCTGTTTGGCC |
| | CAATAGTGAGAACTTTTTCCTGCTGCCTCTTGGTGCTTTTGCCTATGGCCCCTATTCTGCCTGCTGAAGACACTCTTGCCAGCATGGACTTAAACCCCTCCAGCTCTGACAATCCTCTTTCTCTTTTGTTTTACATGAAGGGTCTG |
| | GCAGCCAAAGCAATCACTCAAAGTTCAAACCTTATCATTTTTTGCTTTGTTCCTCTTGGCCTTGGTTTTGTACATCAGCTTTGAAAATACCATCCCAGGGTTAATGCTGGGGTTAATTTATAACTGAGAGTGCTCTAGTTCTGCAA |
| | TACAGGACATGCTATAAAAATGGAAAGATGTTGCTTTCTGAGAGATCAGGTGGATTCTTGGGCATTTGCTTAAGCTTGGTACCGCCACCATGAGAAAAGACAGACTTCTTCATTTATGTCTTGTGCTACTTCTTATCCTGCTTTCT |
| | GCCAGTGACTCAAACTCTCAGCAGGGGGCCAGCAGGCCAGGCCCCAGGGATGCCCAGGCCCACCCTGGCAGACCCAGGGCTGTGCCCACCCAGTGTGATGTGCCTCCCAACAGCAGATTTGACTGTGCCCCTGATAAGGCCATTAC |
| | TCAGGAACAGTGTGAGGCCAGGGGCTGCTGCTATATCCCTGCCAAGCAGGGCCTGCAGGGGGCCCAGATGGGGCAGCCCTGGTGCTTCTTCCCCCCCAGCTACCCCTCTTATAAGCTGGAGAATCTGAGCAGCTCTGAAATGGGCT |
| | ACACTGCCACTCTGACCAGGACTACCCCCACCTTCTTCCCCAAGGATATTCTGACTCTGAGGCTGGATGTGATGATGGAGACTGAGAACAGGCTGCACTTCACCATCAAGGACCCTGCCAACAGGAGGTATGAGGTGCCCCTGGAA |
| | ACTCCCAGGGTGCACTCTAGGGCCCCCAGCCCCCTGTATTCTGTGGAGTTCTCTGAGGAGCCTTTTGGGGTCATTGTCCACAGGCAGCTGGATGGCAGGGTGCTGCTGAATACTACTGTGGCCCCTCTGTTCTTTGCTGACCAGTT |
| | CCTGCAGCTGAGCACCAGCCTGCCTTCTCAGTACATTACTGGCCTGGCTGAGCATCTGAGCCCCCTGATGCTGAGCACCTCTTGGACCAGAATCACCCTGTGGAACAGGGACCTGGCTCCCACTCCTGGGGCCAACCTGTATGGCA |
| | GCCACCCCTTCTACCTGGCCCTGGAGGATGGGGGCTCTGCCCATGGGGTGTTCCTGCTGAACAGCAATGCCATGGATGTGGTGCTGCAGCCCTCTCCTGCCCTGTCTTGGAGATCTACTGGGGGCATCCTGGATGTGTATATCTTC |
| | CTGGGGCCTGAGCCCAAGTCTGTGGTGCAGCAGTACCTGGATGTGGTGGGCTACCCCTTCATGCCCCCCTACTGGGGCCTGGGCTTCCACCTGTGCAGGTGGGGCTACTCTTCTACTGCTATCACCAGGCAGGTGGTGGAGAACAT |
| | GACCAGGGCTCACTTCCCTCTGGATGTGCAGTGGAATGACCTGGACTACATGGACTCTAGGAGAGACTTCACTTTTAATAAGGATGGGTTCAGGGACTTTCCTGCCATGGTGCAGGAGCTGCATCAGGGGGGCAGGAGATATATGA |
| | TGATTGTGGACCCTGCTATTTCTAGCTCTGGCCCTGCTGGCAGCTATAGGCCCTATGATGAGGGGCTGAGGAGGGGGGTGTTCATCACTAATGAGACTGGCCAGCCCCTGATTGGCAAGGTGTGGCCTGGCTCTACTGCCTTCCCT |
| | GATTTCACCAACCCCACTGCCCTGGCCTGGTGGGAGGATATGGTGGCTGAGTTTCATGACCAGGTGCCCTTTGATGGCATGTGGATTGACATGAATGAGCCCAGCAACTTTATCAGGGGCTCTGAAGATGGCTGCCCCAACAATGA |
| | GCTGGAGAACCCCCCCTATGTGCCTGGGGTGGTGGGGGGCACCCTGCAGGCTGCCACCATCTGTGCCAGCAGCCACCAGTTCCTGAGCACCCACTATAACCTGCACAACCTGTATGGCCTGACTGAGGCCATTGCCTCTCACAGGG |
| | CCCTGGTGAAGGCTAGGGGGACTAGGCCCTTTGTGATCAGCAGGTCTACTTTTGCTGGCCATGGCAGGTATGCTGGGCACTGGACTGGGGATGTGTGGTCTAGCTGGGAGCAGCTGGCCAGCTCTGTGCCTGAGATCCTGCAGTTT |
| | AATCTGCTGGGGGTGCCCCTGGTGGGGGCTGATGTGTGTGGCTTCCTGGGCAATACCTCTGAGGAGCTGTGTGTGAGGTGGACTCAGCTGGGGGCTTTCTACCCCTTCATGAGAAACCACAACTCTCTGCTGAGCCTGCCCCAGGA |
| | GCCCTATTCTTTTTCTGAGCCTGCCCAGCAGGCTATGAGGAAGGCCCTGACTCTGAGGTATGCCCTGCTGCCCCACCTGTATACCCTGTTCCATCAGGCCCATGTGGCTGGGGAGACTGTGGCCAGACCTCTGTTCCTGGAGTTCC |
| | CCAAGGATAGCTCTACTTGGACTGTGGACCACCAGCTGCTGTGGGGGGAGGCTCTGCTGATCACCCCTGTGCTGCAGGCTGGGAAGGCTGAGGTGACTGGCTATTTCCCCCTGGGCACCTGGTATGATCTGCAGACTGTGCCCATT |
| | GAGGCCCTGGGCTCTCTGCCCCCTCCCCCTGCTGCCCCCAGGGAGCCTGCCATCCACTCTGAGGGCCAGTGGGTGACCCTGCCTGCCCCTCTGGACACTATCAATGTGCACCTGAGGGCTGGCTACATCATCCCCCTGCAGGGCCC |
| | TGGCCTGACTACCACTGAGTCTAGGCAGCAGCCCATGGCCCTGGCTGTGGCTCTGACCAAGGGGGGGGAGGCCAGGGGGGAGCTGTTCTGGGATGATGGGGAGTCTCTGGAGGTGCTGGAGAGGGGGGCCTACACCCAGGTGATCT |
| | TCCTGGCTAGGAATAACACCATTGTCAATGAGCTGGTGAGGGTGACCTCTGAGGGGGCTGGCCTGCAGCTGCAGAAGGTGACTGTGCTGGGGGTGGCTACTGCCCCCCAGCAGGTGCTGAGCAATGGGGTGCCTGTGAGCAACTTC |
| | ACCTATAGCCCTGACACCAAGGTGCTGGACATCTGTGTGAGCCTGCTGATGGGGGAGCAGTTCCTGGTGAGCTGGTGCTAATTTAAATCTCGAGCCGGGCGGAGTGTGTTAGTCTCTCCAGAGGGAGGCTGGTTCCCCAGGGAAGC |
| | AGAGCCTGTGTGCGGGCAGCAGCTGTGTGCGGGCCTGGGGGTTGTTAAGTGCAATTATTTTTAATAAAAGGGGCATTTGGAAAAAAAAAAAAAAGGTAGCAGTCGACAGATGAATTCTGCAGATCTGTGGCTTCTAGCTGCCCGGG |
| | TGGCATCCCTGTGACCCCTCCCCAGTGCCTCTCCTGGCCCTGGAAGTTGCCACTCCAGTGCCCACCAGCCTTGTCCTAATAAAATTAAGTTGCATCATTTTGTCTGACTAGGTGTCCTTCTATAATATTATGGGGTGGAGGGGGGT |
| | GGTATGGAGCAAGGGGCAAGTTGGGAAGACAACCTGTAGGGCCTGCGGGGTCTATTGGGAACCAAGCTGGAGTGCAGTGGCACAATCTTGGCTCACTGCAATCTCCGCCTCCTGGGTTCAAGCGATTCTCCTGCCTCAGCCTCCCG |
| | AGTTGTTGGGATTCCAGGCATGCATGACCAGGCTCAGCTAATTTTTGTTTTTTTGGTAGAGACGGGGTTTCACCATATTGGCCAGGCTGGTCTCCAACTCCTAATCTCAGGTGATCTACCCACCTTGGCCTCCCAAATTGCTGGGA |
| | TTACAGGCGTGAACCACTGCTCCCTTCCCTGTCCTTCTGATTTTAAAAAAAAGAGAAATGTTCTGGCACCTGCACTTGCACTGGGGACAGCCTATTTTGCTAGTTTGTTTTGTTTCGTTTTGTTTTGATGGAGAGCGTATGTTGTT |
| | TAAACGCGGCCGCGTAGATAAGTAGCATGGCGGGTTAATCATTAACTACAAGGAACCCCTAGTGATGGAGTTGGCCACTCCCTCTCTGCGCGCTCGCTCGCTCACTGAGGCCGGGCGACCAAAGGTCGCCCGACGCCCGGGCTTTG |
| | CCCGGGCGGCCTCAGTGAGCGAGCGAGCGCGCAG |
| 36 | > dbDBA plasmid with M2 LSP and GAA nucleic acid |
| | TCTAGAGCTAGCATATGGATCCATCGATTTAGGGATAACAGGGTAATTATCAGCACACAATTGCCCATTATACGCGCGTATAATGGACTATTGTGTGCTGATATCTGTACACTTAAGGGCTAGATCTTAGCTTACGTCACTAGAG |
| | GGTCCACGTTTAGTTTTTAAGATCCATTGATCTCCTAAACGCTGCAAGATTCGCAACCTGGTATACTTAGCCTAGGCGCTAGGTCCTAGTGCAGCGGGACTTTTTTTCTAAAGTCGTTGAGAGGAGGAGTCGTCAGACCAGATAG |
| | CTTTGATGTCCTGATCGGAAGGATCGTTGGCCCCCCTGCAGGCAGCTGTTAATTAACTGCGCGCTCGCTCGCTCACTGAGGCCGCCCGGGCAAAGCCCGGGCGTCGGGCGACCTTTGGTCGCCCGGCCTCAGTGAGCGAGCGAGC |
| | GCGCAGAGAGGGAGTGGCCAACTCCATCACTAGGGGTTCCTTGTAGTTAATGATTAACCCGCCATGCTACTTATCTACGGCGCGCCACGCGTTCTAGGAGTTAATTTTTAAAAAGCAGTCAAAAGTCCAAGTGCCCTTGCGAGCA |
| | TTTACTCTCTCTGTTTGCTCTGGTTAATAATCTCAGGAGCACAAACATTCCTTACTAGTTCTAGGAGTTAATTTTTAAAAAGCAGTCAAAAGTCCAAGTGCCCTTGCGAGCATTTACTCTCTCTGTTTGCTCTGGTTAATAATCT |
| | CAGGAGCACAAACATTCCTTACTAGTTCTAGAGCGGCCGCCAGTGTGCTGGAATTCGGCTTTTTTAGGGCTGGAAGCTACCTTTGACATCATTTCCTCTGCGAATGCATGTATAATTTCTACAGAACCTATTAGAAAGGATCACC |
| | CAGCCTCTGCTTTTGTACAACTTTCCCTTAAAAAACTGCCAATCCCACTGCTGTTTGGCCCAATAGTGAGAACTTTTTCCTGCTGCCTCTTGGTGCTTTTGCCTATGGCCCCTATTCTGCCTGCTGAAGACACTCTTGCCAGCAT |
| | GGACTTAAACCCCTCCAGCTCTGACAATCCTCTTTCTCTTTTGTTTTACATGAAGGGTCTGGCAGCCAAAGCAATCACTCAAAGTTCAAACCTTATCATTTTTTGCTTTGTTCCTCTTGGCCTTGGTTTTGTACATCAGCTGTGA |
| | AAATACCATCCCAGGGTATTTATAGCTGGGGTTAATTTATAACTGAGAGTGCTCTAGTTCTGCAATACAGGACATGCTATAAAAATGGAAAGATGTTGCTTTCTGAGAGATCAGCTTACATGTGCTAGCGTAGCCAACCATGGGA |
| | GTGAGGCACCCGCCCTGCTCCCACCGGCTCCTGGCCGTCTGCGCCCTCGTGTCCTTGGCAACCGCTGCACTCCTGGGGCACATCCTACTCCATGATTTCCTGCTGGTTCCCCGAGAGCTGAGTGGCTCCTCCCCAGTCCTGGAGG |
| | AGACTCACCCAGCTCACCAGCAGGGAGCCAGCAGACCAGGGCCCCGGGACGCCCAGGCACACCCCGGCCGTCCCAGAGCAGTGCCCACACAGTGCGACGTCCCCCCCAACAGCCGCTTCGATTGCGCCCCTGACAAGGCCATCAC |
| | CCAGGAACAGTGCGAGGCCCGCGGCTGCTGCTACATCCCTGCAAAGCAGGGGCTGCAGGGAGCCCAGATGGGGCAGCCCTGGTGCTTCTTCCCACCCAGCTACCCCAGCTACAAGCTGGAGAACCTGAGCTCCTCTGAAATGGGC |
| | TACACGGCCACCCTGACCCGTACCACCCCCACCTTCTTCCCCAAGGACATCCTGACCCTGCGGCTGGACGTGATGATGGAGACTGAGAACCGCCTCCACTTCACGATCAAAGATCCAGCTAACAGGCGCTACGAGGTGCCCTTGG |
| | AGACCCCGCGTGTCCACAGCCGGGCACCGTCCCCACTCTACAGCGTGGAGTTCTCCGAGGAGCCCTTCGGGGTGATCGTGCACCGGCAGCTGGACGGCCGCGTGCTGCTGAACACGACGGTGGCGCCCCTGTTCTTTGCGGACCA |
| | GTTCCTTCAGCTGTCCACCTCGCTGCCCTCGCAGTATATCACAGGCCTCGCCGAGCACCTCAGTCCCCTGATGCTCAGCACCAGCTGGACCAGGATCACCCTGTGGAACCGGGACCTTGCGCCCACGCCCGGTGCGAACCTCTAC |
| | GGGTCTCACCCTTTCTACCTGGCGCTGGAGGACGGCGGGTCGGCACACGGGGTGTTCCTGCTAAACAGCAACGCCATGGACGTGGTCCTGCAGCCGAGCCCTGCCCTTAGCTGGAGGTCGACAGGTGGGATCCTGGACGTCTACA |
| | TCTTCCTGGGCCCAGAGCCCAAGAGCGTGGTGCAGCAGTACCTGGACGTGGTGGGATACCCGTTCATGCCGCCATACTGGGGCCTGGGCTTCCACCTGTGCCGCTGGGGCTACTCCTCCACCGCTATCACCCGCCAGGTGGTGGA |
| | GAACATGACCAGGGCCCACTTCCCCCTGGACGTCCAGTGGAACGATCTGGACTACATGGACTCCCGGAGGGACTTCACGTTCAACAAGGATGGCTTCCGGGACTTCCCGGCCATGGTGCAGGAGCTGCACCAGGGCGGCCGGCGC |
| | TACATGATGATCGTGGATCCTGCCATCAGCAGCTCGGGCCCTGCCGGGAGCTACAGGCCCTACGACGAGGGTCTGCGGAGGGGGGTTTTCATCACCAACGAGACCGGCCAGCCGCTGATTGGGAAGGTGTGGCCCGGGTCCACTG |
| | CCTTCCCCGACTTCACCAACCCCACAGCCCTGGCCTGGTGGGAGGACATGGTGGCTGAGTTCCATGACCAGGTGCCCTTCGACGGCATGTGGATTGACATGAACGAGCCTTCCAACTTCATCAGAGGCTCTGAGGACGGCTGCCC |
| | CAACAACGAGCTGGAGAACCCACCCTACGTGCCTGGGGTGGTTGGGGGGACCCTCCAGGCGGCCACCATCTGTGCCTCCAGCCACCAGTTTCTCTCCACACACTACAACCTGCACAACCTCTACGGCCTGACCGAAGCCATCGCC |
| | TCCCACAGGGCGCTGGTGAAGGCTCGGGGGACACGCCCATTTGTGATCTCCCGCTCGACCTTTGCTGGCCACGGCCGATACGCCGGCCACTGGACGGGGGACGTGTGGAGCTCCTGGGAGCAGCTCGCCTCCTCCGTGCCAGAAA |
| | TCCTGCAGTTTAACCTGCTGGGGGTGCCTCTGGTCGGGGCCGACGTCTGCGGCTTCCTGGGCAACACCTCAGAGGAGCTGTGTGTGCGCTGGACCCAGCTGGGGGCCTTCTACCCCTTCATGCGGAACCACAACAGCCTGCTCAG |
| | TCTGCCCCAGGAGCCGTACAGCTTCAGCGAGCCGGCCCAGCAGGCCATGAGGAAGGCCCTCACCCTGCGCTACGCACTCCTCCCCCACCTCTACACACTGTTCCACCAGGCCCACGTCGCGGGGGAGACCGTGGCCCGGCCCCTC |
| | TTCCTGGAGTTCCCCAAGGACTCTAGCACCTGGACTGTGGACCACCAGCTCCTGTGGGGGGAGGCCCTGCTCATCACCCCAGTGCTCCAGGCCGGGAAGGCCGAAGTGACTGGCTACTTCCCCTTGGGCACATGGTACGACCTGC |
| | AGACGGTGCCAATAGAGGCCCTTGGCAGCCTCCCACCCCCACCTGCAGCTCCCCGTGAGCCAGCCATCCACAGCGAGGGGCAGTGGGTGACGCTGCCGGCCCCCCTGGACACCATCAACGTGCACCTCCGGGCTGGGTACATCAT |
| | CCCCCTGCAGGGCCCTGGCCTCACAACCACAGAGTCCCGCCAGCAGCCCATGGCCCTGGCTGTGGCCCTGACCAAGGGTGGAGAGGCCCGAGGGGAGCTGTTCTGGGACGACGGAGAGAGCCTGGAAGTGCTGGAGCGAGGGGCC |
| | TACACACAGGTCATCTTCCTGGCCAGGAATAACACGATCGTGAATGAGCTGGTACGTGTGACCAGTGAGGGAGCTGGCCTGCAGCTGCAGAAGGTGACTGTCCTGGGCGTGGCCACGGCGCCCCAGCAGGTCCTCTCCAACGGTG |
| | TCCCTGTCTCCAACTTCACCTACAGCCCCGACACCAAGGTCCTGGACATCTGTGTCTCGCTGTTGATGGGAGAGCAGTTTCTCGTCAGCTGGTGTTGATAACTCGAGCCGGGCGGAGTGTGTTAGTCTCTCCAGAGGGAGGCTGG |
| | TTCCCCAGGGAAGCAGAGCCTGTGTGCGGGCAGCAGCTGTGTGCGGGCCTGGGGGTTGTTAAGTGCAATTATTTTTAATAAAAGGGGCATTTGGAAAAAAAAAAAAAAGGTAGCAGTCGACAGATGAATTCTGCAGATCTGTGGC |
| | TTCTAGCTGCCCGGGTGGCATCCCTGTGACCCCTCCCCAGTGCCTCTCCTGGCCCTGGAAGTTGCCACTCCAGTGCCCACCAGCCTTGTCCTAATAAAATTAAGTTGCATCATTTTGTCTGACTAGGTGTCCTTCTATAATATTA |
| | TGGGGTGGAGGGGGGTGGTATGGAGCAAGGGGCAAGTTGGGAAGACAACCTGTAGGGCCTGCGGGGTCTATTGGGAACCAAGCTGGAGTGCAGTGGCACAATCTTGGCTCACTGCAATCTCCGCCTCCTGGGTTCAAGCGATTCT |
| | CCTGCCTCAGCCTCCCGAGTTGTTGGGATTCCAGGCATGCATGACCAGGCTCAGCTAATTTTTGTTTTTTTGGTAGAGACGGGGTTTCACCATATTGGCCAGGCTGGTCTCCAACTCCTAATCTCAGGTGATCTACCCACCTTGG |
| | CCTCCCAAATTGCTGGGATTACAGGCGTGAACCACTGCTCCCTTCCCTGTCCTTCTGATTTTAAAAAAAAGAGAAATGTTCTGGCACCTGCACTTGCACTGGGGACAGCCTATTTTGCTAGTTTGTTTTGTTTCGTTTTGTTTTG |
| | ATGGAGAGCGTATGTTGTTTAAACGCGGCCGCGTAGATAAGTAGCATGGCGGGTTAATCATTAACTACAAGGAACCCCTAGTGATGGAGTTGGCCACTCCCTCTCTGCGCGCTCGCTCGCTCACTGAGGCCGGGCGACCAAAGGT |
| | CGCCCGACGCCCGGGCTTTGCCCGGGCGGCCTCAGTGAGCGAGCGAGCGCGCAGTTAATTAAGGCGCCCTAGGCCGACCCTTAGACTCTGTACTCAGTTCTATAAACGAGCCATTGGATACGAGATCCGTAGATTGATAAGGGAC |
| | ACGGAATATCCCCGGACGCAATAGACACCGGTGGACAGCTTGGTATCCTGAGCACAGTCGCGCGTCCGAATCTAGCTCTACTTTAGAGGCCCCGGATTCTGATGGTCGTAGACCGCAGAACCGATTGGGGGGATGAGATCTACTA |
| | GTTATCAGCACACAATTGCCCATTATACGCGCGTATAATGGACTATTGTGTGCTGATATAGGGATAACAGGGTAATTCTAGAGCTAGCATATGGATCCATCGATTTGATGCGGTATTTTCTCCTTACGCATCTGTGCGGTATTTC |
| | ACACCGCATACGTCAAAGCAACCATAGTACGCGCCCTGTAGCGGCGCATTAAGCGCGGCGGGTGTGGTGGTTACGCGCAGCGTGACCGCTACACTTGCCAGCGCCCTAGCGCCCGCTCCTTTCGCTTTCTTCCCTTCCTTTCTCG |
| | CCACGTTCGCCGGCTTTCCCCGTCAAGCTCTAAATCGGGGGCTCCCTTTAGGGTTCCGATTTAGTGCTTTACGGCACCTCGACCCCAAAAAACTTGATTTGGGTGATGGTTCACGTAGTGGGCCATCGCCCTGATAGACGGTTTT |
| | TCGCCCTTTGACGTTGGAGTCCACGTTCTTTAATAGTGGACTCTTGTTCCAAACTGGAACAACACTCAACTCTATCTCGGGCTATTCTTTTGATTTATAAGGGATTTTGCCGATTTCGGTCTATTGGTTAAAAAATGAGCTGATT |
| | TAACAAAAATTTAACGCGAATTTTAACAAAATATTAACGTTTACAATTTTATGGTGCACTCTCAGTACAATCTGCTCTGATGCCGCATAGTTAAGCCAGCCCCGACACCCGCCAACACCCGCTGACGCGCCCTGACGGGCTTGTC |
| | TGCTCCCGGCATCCGCTTACAGACAAGCTGTGACCGTCTCCGGGAGCTGCATGTGTCAGAGGTTTTCACCGTCATCACCGAAACGCGCGAGACGAAAGGGCCTCGTGATACGCCTATTTTTATAGGTTAATGTCATGATAATAAT |
| | GGTTTCTTAGACGTCAGGTGGCACTTTTCGGGGAAATGTGCGCGGAACCCCTATTTGTTTATTTTTCTAAATACATTCAAATATGTATCCGCTCATGAGACAATAACCCTGATAAATGCTTCAATAATATTGAAAAAGGAAGAGT |
| | ATGAGCCATATTCAACGGGAAACGTCGAGGCCGCGATTAAATTCCAACATGGATGCTGATTTATATGGGTATAAATGGGCTCGCGATAATGTCGGGCAATCAGGTGCGACAATCTATCGCTTGTATGGGAAGCCCGATGCGCCAG |
| | AGTTGTTTCTGAAACATGGCAAAGGTAGCGTTGCCAATGATGTTACAGATGAGATGGTCAGACTAAACTGGCTGACGGAATTTATGCCTCTTCCGACCATCAAGCATTTTATCCGTACTCCTGATGATGCATGGTTACTCACCAC |
| | TGCGATCCCCGGAAAAACAGCATTCCAGGTATTAGAAGAATATCCTGATTCAGGTGAAAATATTGTTGATGCGCTGGCAGTGTTCCTGCGCCGGTTGCATTCGATTCCTGTTTGTAATTGTCCTTTTAACAGCGATCGCGTATTT |
| | CGTCTCGCTCAGGCGCAATCACGAATGAATAACGGTTTGGTTGATGCGAGTGATTTTGATGACGAGCGTAATGGCTGGCCTGTTGAACAAGTCTGGAAAGAAATGCATAAACTTTTGCCATTCTCACCGGATTCAGTCGTCACTC |
| | ATGGTGATTTCTCACTTGATAACCTTATTTTTGACGAGGGGAAATTAATAGGTTGTATTGATGTTGGACGAGTCGGAATCGCAGACCGATACCAGGATCTTGCCATCCTATGGAACTGCCTCGGTGAGTTTTCTCCTTCATTACA |
| | GAAACGGCTTTTTCAAAAATATGGTATTGATAATCCTGATATGAATAAATTGCAGTTTCATTTGATGCTCGATGAGTTTTTCTAAGCGTATAATGGTCTAGAGCTAGCATATGGATCCATCGATTCCATTATACGCCTGTCAGAC |
| | CAAGTTTACTCATATATACTTTAGATTGATTTAAAACTTCATTTTTAATTTAAAAGGATCTAGGTGAAGATCCTTTTTGATAATCTCATGACCAAAATCCCTTAACGTGAGTTTTCGTTCCACTGAGCGTCAGACCCCGTAGAAA |
| | AGATCAAAGGATCTTCTTGAGATCCTTTTTTTCTGCGCGTAATCTGCTGCTTGCAAACAAAAAAACCACCGCTACCAGCGGTGGTTTGTTTGCCGGATCAAGAGCTACCAACTCTTTTTCCGAAGGTAACTGGCTTCAGCAGAGC |
| | GCAGATACCAAATACTGTTCTTCTAGTGTAGCCGTAGTTAGGCCACCACTTCAAGAACTCTGTAGCACCGCCTACATACCTCGCTCTGCTAATCCTGTTACCAGTGGCTGCTGCCAGTGGCGATAAGTCGTGTCTTACCGGGTTG |
| | GACTCAAGACGATAGTTACCGGATAAGGCGCAGCGGTCGGGCTGAACGGGGGGTTCGTGCACACAGCCCAGCTTGGAGCGAACGACCTACACCGAACTGAGATACCTACAGCGTGAGCTATGAGAAAGCGCCACGCTTCCCGAAG |
| | GGAGAAAGGCGGACAGGTATCCGGTAAGCGGCAGGGTCGGAACAGGAGAGCGCACGAGGGAGCTTCCAGGGGGAAACGCCTGGTATCTTTATAGTCCTGTCGGGTTTCGCCACCTCTGACTTGAGCGTCGATTTTTGTGATGCTC |
| | GTCAGGGGGGCGGAGCCTATGGAAAAACGCCAGCAACGCGGCCTTTTTACGGTTCCTGGCCTTTTGCTGGCCTTTTGCTCACATGT |
| 37 | > dbDBA plasmid with M3 LSP and GAA nucleic acid |
| | TCTAGAGCTAGCATATGGATCCATCGATTTAGGGATAACAGGGTAATTATCAGCACACAATTGCCCATTATACGCGCGTATAATGGACTATTGTGTGCTGATATCTGTACACTTAAGGGCTAGATCTTAGCTTACGTCACTAGAGG |
| | GTCCACGTTTAGTTTTTAAGATCCATTGATCTCCTAAACGCTGCAAGATTCGCAACCTGGTATACTTAGCCTAGGCGCTAGGTCCTAGTGCAGCGGGACTTTTTTTCTAAAGTCGTTGAGAGGAGGAGTCGTCAGACCAGATAGCT |
| | TTGATGTCCTGATCGGAAGGATCGTTGGCCCCCCTGCAGGCAGCTGTTAATTAACTGCGCGCTCGCTCGCTCACTGAGGCCGCCCGGGCAAAGCCCGGGCGTCGGGCGACCTTTGGTCGCCCGGCCTCAGTGAGCGAGCGAGCGCG |
| | CAGAGAGGGAGTGGCCAACTCCATCACTAGGGGTTCCTTGTAGTTAATGATTAACCCGCCATGCTACTTATCTACGGCGCGCCACGCGTTCTAGGAGTTAATTTTTAAAAAGCAGTCAAAAGTCCAAGTGCCCTTGCGAGCATTTA |
| | CTCTCTCTGTTTGCTCTGGTTAATAATCTCAGGAGCACAAACATTCCTTACTAGTTCTAGGAGTTAATTTTTAAAAAGCAGTCAAAAGTCCAAGTGCCCTTGCGAGCATTTACTCTCTCTGTTTGCTCTGGTTAATAATCTCAGGA |
| | GCACAAACATTCCTTACTAGTTCTAGAGCGGCCGCCAGTGTGCTGGAATTCGGCTTTTTTAGGGCTGGAAGCTACCTTTGACATCATTTCCTCTGCGAATGCATGTATAATTTCTACAGAACCTATTAGAAAGGATCACCCAGCCT |
| | CTGCTTTTGTACAACTTTCCCTTAAAAAACTGCCAATCCCACTGCTGTTTGGCCCAATAGTGAGAACTTTTTCCTGCTGCCTCTTGGTGCTTTTGCCTATGGCCCCTATTCTGCCTGCTGAAGACACTCTTGCCAGCATGGACTTA |
| | AACCCCTCCAGCTCTGACAATCCTCTTTCTCTTTTGTTTTACATGAAGGGTCTGGCAGCCAAAGCAATCACTCAAAGTTCAAACCTTATCATTTTTTGCTTTGTTCCTCTTGGCCTTGGTTTTGTACATCAGCTTTGAAAATACCA |
| | TCCCAGGGTTAATGCTGGGGTTAATTTATAACTGAGAGTGCTCTAGTTCTGCAATACAGGACATGCTATAAAAATGGAAAGATGTTGCTTTCTGAGAGATCAGCTTACATGTGCTAGCGTAGCCAACCATGGGAGTGAGGCACCCG |
| | CCCTGCTCCCACCGGCTCCTGGCCGTCTGCGCCCTCGTGTCCTTGGCAACCGCTGCACTCCTGGGGCACATCCTACTCCATGATTTCCTGCTGGTTCCCCGAGAGCTGAGTGGCTCCTCCCCAGTCCTGGAGGAGACTCACCCAGC |
| | TCACCAGCAGGGAGCCAGCAGACCAGGGCCCCGGGACGCCCAGGCACACCCCGGCCGTCCCAGAGCAGTGCCCACACAGTGCGACGTCCCCCCCAACAGCCGCTTCGATTGCGCCCCTGACAAGGCCATCACCCAGGAACAGTGCG |
| | AGGCCCGCGGCTGCTGCTACATCCCTGCAAAGCAGGGGCTGCAGGGAGCCCAGATGGGGCAGCCCTGGTGCTTCTTCCCACCCAGCTACCCCAGCTACAAGCTGGAGAACCTGAGCTCCTCTGAAATGGGCTACACGGCCACCCTG |
| | ACCCGTACCACCCCCACCTTCTTCCCCAAGGACATCCTGACCCTGCGGCTGGACGTGATGATGGAGACTGAGAACCGCCTCCACTTCACGATCAAAGATCCAGCTAACAGGCGCTACGAGGTGCCCTTGGAGACCCCGCGTGTCCA |
| | CAGCCGGGCACCGTCCCCACTCTACAGCGTGGAGTTCTCCGAGGAGCCCTTCGGGGTGATCGTGCACCGGCAGCTGGACGGCCGCGTGCTGCTGAACACGACGGTGGCGCCCCTGTTCTTTGCGGACCAGTTCCTTCAGCTGTCCA |
| | CCTCGCTGCCCTCGCAGTATATCACAGGCCTCGCCGAGCACCTCAGTCCCCTGATGCTCAGCACCAGCTGGACCAGGATCACCCTGTGGAACCGGGACCTTGCGCCCACGCCCGGTGCGAACCTCTACGGGTCTCACCCTTTCTAC |
| | CTGGCGCTGGAGGACGGCGGGTCGGCACACGGGGTGTTCCTGCTAAACAGCAACGCCATGGACGTGGTCCTGCAGCCGAGCCCTGCCCTTAGCTGGAGGTCGACAGGTGGGATCCTGGACGTCTACATCTTCCTGGGCCCAGAGCC |
| | CAAGAGCGTGGTGCAGCAGTACCTGGACGTGGTGGGATACCCGTTCATGCCGCCATACTGGGGCCTGGGCTTCCACCTGTGCCGCTGGGGCTACTCCTCCACCGCTATCACCCGCCAGGTGGTGGAGAACATGACCAGGGCCCACT |
| | TCCCCCTGGACGTCCAGTGGAACGATCTGGACTACATGGACTCCCGGAGGGACTTCACGTTCAACAAGGATGGCTTCCGGGACTTCCCGGCCATGGTGCAGGAGCTGCACCAGGGCGGCCGGCGCTACATGATGATCGTGGATCCT |
| | GCCATCAGCAGCTCGGGCCCTGCCGGGAGCTACAGGCCCTACGACGAGGGTCTGCGGAGGGGGGTTTTCATCACCAACGAGACCGGCCAGCCGCTGATTGGGAAGGTGTGGCCCGGGTCCACTGCCTTCCCCGACTTCACCAACCC |
| | CACAGCCCTGGCCTGGTGGGAGGACATGGTGGCTGAGTTCCATGACCAGGTGCCCTTCGACGGCATGTGGATTGACATGAACGAGCCTTCCAACTTCATCAGAGGCTCTGAGGACGGCTGCCCCAACAACGAGCTGGAGAACCCAC |
| | CCTACGTGCCTGGGGTGGTTGGGGGGACCCTCCAGGCGGCCACCATCTGTGCCTCCAGCCACCAGTTTCTCTCCACACACTACAACCTGCACAACCTCTACGGCCTGACCGAAGCCATCGCCTCCCACAGGGCGCTGGTGAAGGCT |
| | CGGGGGACACGCCCATTTGTGATCTCCCGCTCGACCTTTGCTGGCCACGGCCGATACGCCGGCCACTGGACGGGGGACGTGTGGAGCTCCTGGGAGCAGCTCGCCTCCTCCGTGCCAGAAATCCTGCAGTTTAACCTGCTGGGGGT |
| | GCCTCTGGTCGGGGCCGACGTCTGCGGCTTCCTGGGCAACACCTCAGAGGAGCTGTGTGTGCGCTGGACCCAGCTGGGGGCCTTCTACCCCTTCATGCGGAACCACAACAGCCTGCTCAGTCTGCCCCAGGAGCCGTACAGCTTCA |
| | GCGAGCCGGCCCAGCAGGCCATGAGGAAGGCCCTCACCCTGCGCTACGCACTCCTCCCCCACCTCTACACACTGTTCCACCAGGCCCACGTCGCGGGGGAGACCGTGGCCCGGCCCCTCTTCCTGGAGTTCCCCAAGGACTCTAGC |
| | ACCTGGACTGTGGACCACCAGCTCCTGTGGGGGGAGGCCCTGCTCATCACCCCAGTGCTCCAGGCCGGGAAGGCCGAAGTGACTGGCTACTTCCCCTTGGGCACATGGTACGACCTGCAGACGGTGCCAATAGAGGCCCTTGGCAG |
| | CCTCCCACCCCCACCTGCAGCTCCCCGTGAGCCAGCCATCCACAGCGAGGGGCAGTGGGTGACGCTGCCGGCCCCCCTGGACACCATCAACGTGCACCTCCGGGCTGGGTACATCATCCCCCTGCAGGGCCCTGGCCTCACAACCA |
| | CAGAGTCCCGCCAGCAGCCCATGGCCCTGGCTGTGGCCCTGACCAAGGGTGGAGAGGCCCGAGGGGAGCTGTTCTGGGACGACGGAGAGAGCCTGGAAGTGCTGGAGCGAGGGGCCTACACACAGGTCATCTTCCTGGCCAGGAAT |
| | AACACGATCGTGAATGAGCTGGTACGTGTGACCAGTGAGGGAGCTGGCCTGCAGCTGCAGAAGGTGACTGTCCTGGGCGTGGCCACGGCGCCCCAGCAGGTCCTCTCCAACGGTGTCCCTGTCTCCAACTTCACCTACAGCCCCGA |
| | CACCAAGGTCCTGGACATCTGTGTCTCGCTGTTGATGGGAGAGCAGTTTCTCGTCAGCTGGTGTTGATAACTCGAGCCGGGCGGAGTGTGTTAGTCTCTCCAGAGGGAGGCTGGTTCCCCAGGGAAGCAGAGCCTGTGTGCGGGCA |
| | GCAGCTGTGTGCGGGCCTGGGGGTTGTTAAGTGCAATTATTTTTAATAAAAGGGGCATTTGGAAAAAAAAAAAAAAGGTAGCAGTCGACAGATGAATTCTGCAGATCTGTGGCTTCTAGCTGCCCGGGTGGCATCCCTGTGACCCC |
| | TCCCCAGTGCCTCTCCTGGCCCTGGAAGTTGCCACTCCAGTGCCCACCAGCCTTGTCCTAATAAAATTAAGTTGCATCATTTTGTCTGACTAGGTGTCCTTCTATAATATTATGGGGTGGAGGGGGGTGGTATGGAGCAAGGGGCA |
| | AGTTGGGAAGACAACCTGTAGGGCCTGCGGGGTCTATTGGGAACCAAGCTGGAGTGCAGTGGCACAATCTTGGCTCACTGCAATCTCCGCCTCCTGGGTTCAAGCGATTCTCCTGCCTCAGCCTCCCGAGTTGTTGGGATTCCAGG |
| | CATGCATGACCAGGCTCAGCTAATTTTTGTTTTTTTGGTAGAGACGGGGTTTCACCATATTGGCCAGGCTGGTCTCCAACTCCTAATCTCAGGTGATCTACCCACCTTGGCCTCCCAAATTGCTGGGATTACAGGCGTGAACCACT |
| | GCTCCCTTCCCTGTCCTTCTGATTTTAAAAAAAAGAGAAATGTTCTGGCACCTGCACTTGCACTGGGGACAGCCTATTTTGCTAGTTTGTTTTGTTTCGTTTTGTTTTGATGGAGAGCGTATGTTGTTTAAACGCGGCCGCGTAGA |
| | TAAGTAGCATGGCGGGTTAATCATTAACTACAAGGAACCCCTAGTGATGGAGTTGGCCACTCCCTCTCTGCGCGCTCGCTCGCTCACTGAGGCCGGGCGACCAAAGGTCGCCCGACGCCCGGGCTTTGCCCGGGCGGCCTCAGTGA |
| | GCGAGCGAGCGCGCAGTTAATTAAGGCGCCCTAGGCCGACCCTTAGACTCTGTACTCAGTTCTATAAACGAGCCATTGGATACGAGATCCGTAGATTGATAAGGGACACGGAATATCCCCGGACGCAATAGACACCGGTGGACAGC |
| | TTGGTATCCTGAGCACAGTCGCGCGTCCGAATCTAGCTCTACTTTAGAGGCCCCGGATTCTGATGGTCGTAGACCGCAGAACCGATTGGGGGGATGAGATCTACTAGTTATCAGCACACAATTGCCCATTATACGCGCGTATAATG |
| | GACTATTGTGTGCTGATATAGGGATAACAGGGTAATTCTAGAGCTAGCATATGGATCCATCGATTTGATGCGGTATTTTCTCCTTACGCATCTGTGCGGTATTTCACACCGCATACGTCAAAGCAACCATAGTACGCGCCCTGTAG |
| | CGGCGCATTAAGCGCGGCGGGTGTGGTGGTTACGCGCAGCGTGACCGCTACACTTGCCAGCGCCCTAGCGCCCGCTCCTTTCGCTTTCTTCCCTTCCTTTCTCGCCACGTTCGCCGGCTTTCCCCGTCAAGCTCTAAATCGGGGGC |
| | TCCCTTTAGGGTTCCGATTTAGTGCTTTACGGCACCTCGACCCCAAAAAACTTGATTTGGGTGATGGTTCACGTAGTGGGCCATCGCCCTGATAGACGGTTTTTCGCCCTTTGACGTTGGAGTCCACGTTCTTTAATAGTGGACTC |
| | TTGTTCCAAACTGGAACAACACTCAACTCTATCTCGGGCTATTCTTTTGATTTATAAGGGATTTTGCCGATTTCGGTCTATTGGTTAAAAAATGAGCTGATTTAACAAAAATTTAACGCGAATTTTAACAAAATATTAACGTTTAC |
| | AATTTTATGGTGCACTCTCAGTACAATCTGCTCTGATGCCGCATAGTTAAGCCAGCCCCGACACCCGCCAACACCCGCTGACGCGCCCTGACGGGCTTGTCTGCTCCCGGCATCCGCTTACAGACAAGCTGTGACCGTCTCCGGGA |
| | GCTGCATGTGTCAGAGGTTTTCACCGTCATCACCGAAACGCGCGAGACGAAAGGGCCTCGTGATACGCCTATTTTTATAGGTTAATGTCATGATAATAATGGTTTCTTAGACGTCAGGTGGCACTTTTCGGGGAAATGTGCGCGGA |
| | ACCCCTATTTGTTTATTTTTCTAAATACATTCAAATATGTATCCGCTCATGAGACAATAACCCTGATAAATGCTTCAATAATATTGAAAAAGGAAGAGTATGAGCCATATTCAACGGGAAACGTCGAGGCCGCGATTAAATTCCAA |
| | CATGGATGCTGATTTATATGGGTATAAATGGGCTCGCGATAATGTCGGGCAATCAGGTGCGACAATCTATCGCTTGTATGGGAAGCCCGATGCGCCAGAGTTGTTTCTGAAACATGGCAAAGGTAGCGTTGCCAATGATGTTACAG |
| | ATGAGATGGTCAGACTAAACTGGCTGACGGAATTTATGCCTCTTCCGACCATCAAGCATTTTATCCGTACTCCTGATGATGCATGGTTACTCACCACTGCGATCCCCGGAAAAACAGCATTCCAGGTATTAGAAGAATATCCTGAT |
| | TCAGGTGAAAATATTGTTGATGCGCTGGCAGTGTTCCTGCGCCGGTTGCATTCGATTCCTGTTTGTAATTGTCCTTTTAACAGCGATCGCGTATTTCGTCTCGCTCAGGCGCAATCACGAATGAATAACGGTTTGGTTGATGCGAG |
| | TGATTTTGATGACGAGCGTAATGGCTGGCCTGTTGAACAAGTCTGGAAAGAAATGCATAAACTTTTGCCATTCTCACCGGATTCAGTCGTCACTCATGGTGATTTCTCACTTGATAACCTTATTTTTGACGAGGGGAAATTAATAG |
| | GTTGTATTGATGTTGGACGAGTCGGAATCGCAGACCGATACCAGGATCTTGCCATCCTATGGAACTGCCTCGGTGAGTTTTCTCCTTCATTACAGAAACGGCTTTTTCAAAAATATGGTATTGATAATCCTGATATGAATAAATTG |
| | CAGTTTCATTTGATGCTCGATGAGTTTTTCTAAGCGTATAATGGTCTAGAGCTAGCATATGGATCCATCGATTCCATTATACGCCTGTCAGACCAAGTTTACTCATATATACTTTAGATTGATTTAAAACTTCATTTTTAATTTAA |
| | AAGGATCTAGGTGAAGATCCTTTTTGATAATCTCATGACCAAAATCCCTTAACGTGAGTTTTCGTTCCACTGAGCGTCAGACCCCGTAGAAAAGATCAAAGGATCTTCTTGAGATCCTTTTTTTCTGCGCGTAATCTGCTGCTTGC |
| | AAACAAAAAAACCACCGCTACCAGCGGTGGTTTGTTTGCCGGATCAAGAGCTACCAACTCTTTTTCCGAAGGTAACTGGCTTCAGCAGAGCGCAGATACCAAATACTGTTCTTCTAGTGTAGCCGTAGTTAGGCCACCACTTCAAG |
| | AACTCTGTAGCACCGCCTACATACCTCGCTCTGCTAATCCTGTTACCAGTGGCTGCTGCCAGTGGCGATAAGTCGTGTCTTACCGGGTTGGACTCAAGACGATAGTTACCGGATAAGGCGCAGCGGTCGGGCTGAACGGGGGGTTC |
| | GTGCACACAGCCCAGCTTGGAGCGAACGACCTACACCGAACTGAGATACCTACAGCGTGAGCTATGAGAAAGCGCCACGCTTCCCGAAGGGAGAAAGGCGGACAGGTATCCGGTAAGCGGCAGGGTCGGAACAGGAGAGCGCACGA |
| | GGGAGCTTCCAGGGGGAAACGCCTGGTATCTTTATAGTCCTGTCGGGTTTCGCCACCTCTGACTTGAGCGTCGATTTTTGTGATGCTCGTCAGGGGGGGGAGCCTATGGAAAAACGCCAGCAACGCGGCCTTTTTACGGTTCCTG |
| | GCCTTTTGCTGGCCTTTTGCTCACATGT |
| 38 | > coGAA nucleic acid sequence w/o signal peptide |
| | CAGCAGGGGGCCAGCAGGCCAGGCCCCAGGGATGCCCAGGCCCACCCTGGCAGACCCAGGGCTGTGCCCACCCAGTGTGATGTGCCTCCCAACAGCAGATTTGACTGTGCCCCTGATAAGGCCATTACTCAGGAACAGTGTGAGGC |
| | CAGGGGCTGCTGCTATATCCCTGCCAAGCAGGGCCTGCAGGGGGCCCAGATGGGGCAGCCCTGGTGCTTCTTCCCCCCCAGCTACCCCTCTTATAAGCTGGAGAATCTGAGCAGCTCTGAAATGGGCTACACTGCCACTCTGACCA |
| | GGACTACCCCCACCTTCTTCCCCAAGGATATTCTGACTCTGAGGCTGGATGTGATGATGGAGACTGAGAACAGGCTGCACTTCACCATCAAGGACCCTGCCAACAGGAGGTATGAGGTGCCCCTGGAAACTCCCAGGGTGCACTCT |
| | AGGGCCCCCAGCCCCCTGTATTCTGTGGAGTTCTCTGAGGAGCCTTTTGGGGTCATTGTCCACAGGCAGCTGGATGGCAGGGTGCTGCTGAATACTACTGTGGCCCCTCTGTTCTTTGCTGACCAGTTCCTGCAGCTGAGCACCAG |
| | CCTGCCTTCTCAGTACATTACTGGCCTGGCTGAGCATCTGAGCCCCCTGATGCTGAGCACCTCTTGGACCAGAATCACCCTGTGGAACAGGGACCTGGCTCCCACTCCTGGGGCCAACCTGTATGGCAGCCACCCCTTCTACCTGG |
| | CCCTGGAGGATGGGGGCTCTGCCCATGGGGTGTTCCTGCTGAACAGCAATGCCATGGATGTGGTGCTGCAGCCCTCTCCTGCCCTGTCTTGGAGATCTACTGGGGGCATCCTGGATGTGTATATCTTCCTGGGGCCTGAGCCCAAG |
| | TCTGTGGTGCAGCAGTACCTGGATGTGGTGGGCTACCCCTTCATGCCCCCCTACTGGGGCCTGGGCTTCCACCTGTGCAGGTGGGGCTACTCTTCTACTGCTATCACCAGGCAGGTGGTGGAGAACATGACCAGGGCTCACTTCCC |
| | TCTGGATGTGCAGTGGAATGACCTGGACTACATGGACTCTAGGAGAGACTTCACTTTTAATAAGGATGGGTTCAGGGACTTTCCTGCCATGGTGCAGGAGCTGCATCAGGGGGGCAGGAGATATATGATGATTGTGGACCCTGCTA |
| | TTTCTAGCTCTGGCCCTGCTGGCAGCTATAGGCCCTATGATGAGGGGCTGAGGAGGGGGGTGTTCATCACTAATGAGACTGGCCAGCCCCTGATTGGCAAGGTGTGGCCTGGCTCTACTGCCTTCCCTGATTTCACCAACCCCACT |
| | GCCCTGGCCTGGTGGGAGGATATGGTGGCTGAGTTTCATGACCAGGTGCCCTTTGATGGCATGTGGATTGACATGAATGAGCCCAGCAACTTTATCAGGGGCTCTGAAGATGGCTGCCCCAACAATGAGCTGGAGAACCCCCCCTA |
| | TGTGCCTGGGGTGGTGGGGGGCACCCTGCAGGCTGCCACCATCTGTGCCAGCAGCCACCAGTTCCTGAGCACCCACTATAACCTGCACAACCTGTATGGCCTGACTGAGGCCATTGCCTCTCACAGGGCCCTGGTGAAGGCTAGGG |
| | GGACTAGGCCCTTTGTGATCAGCAGGTCTACTTTTGCTGGCCATGGCAGGTATGCTGGGCACTGGACTGGGGATGTGTGGTCTAGCTGGGAGCAGCTGGCCAGCTCTGTGCCTGAGATCCTGCAGTTTAATCTGCTGGGGGTGCCC |
| | CTGGTGGGGGCTGATGTGTGTGGCTTCCTGGGCAATACCTCTGAGGAGCTGTGTGTGAGGTGGACTCAGCTGGGGGCTTTCTACCCCTTCATGAGAAACCACAACTCTCTGCTGAGCCTGCCCCAGGAGCCCTATTCTTTTTCTGA |
| | GCCTGCCCAGCAGGCTATGAGGAAGGCCCTGACTCTGAGGTATGCCCTGCTGCCCCACCTGTATACCCTGTTCCATCAGGCCCATGTGGCTGGGGAGACTGTGGCCAGACCTCTGTTCCTGGAGTTCCCCAAGGATAGCTCTACTT |
| | GGACTGTGGACCACCAGCTGCTGTGGGGGGAGGCTCTGCTGATCACCCCTGTGCTGCAGGCTGGGAAGGCTGAGGTGACTGGCTATTTCCCCCTGGGCACCTGGTATGATCTGCAGACTGTGCCCATTGAGGCCCTGGGCTCTCTG |
| | CCCCCTCCCCCTGCTGCCCCCAGGGAGCCTGCCATCCACTCTGAGGGCCAGTGGGTGACCCTGCCTGCCCCTCTGGACACTATCAATGTGCACCTGAGGGCTGGCTACATCATCCCCCTGCAGGGCCCTGGCCTGACTACCACTGA |
| | GTCTAGGCAGCAGCCCATGGCCCTGGCTGTGGCTCTGACCAAGGGGGGGGAGGCCAGGGGGGAGCTGTTCTGGGATGATGGGGAGTCTCTGGAGGTGCTGGAGAGGGGGGCCTACACCCAGGTGATCTTCCTGGCTAGGAATAACA |
| | CCATTGTCAATGAGCTGGTGAGGGTGACCTCTGAGGGGGCTGGCCTGCAGCTGCAGAAGGTGACTGTGCTGGGGGTGGCTACTGCCCCCCAGCAGGTGCTGAGCAATGGGGTGCCTGTGAGCAACTTCACCTATAGCCCTGACACC |
| | AAGGTGCTGGACATCTGTGTGAGCCTGC! TGATGGGGGAGCAGTTCCTGGTGAGCTGGTGCTAA |
| 39 | > coGAA GAA nucleic acid |
| | ATGGGAGTGAGGCACCCCCCCTGCTCCCATAGACTCCTGGCTGTCTGTGCCCTGGTGTCCTTGGCAACAGCTGCACTC |
| | CAGCAGGGGGCCAGCAGGCCAGGCCCCAGGGATGCCCAGGCCCACCCTGGCAGACCCAGGGCTGTGCCCACCCAGTGTGATGTGCCTCCCAACAGCAGATTTGACTGTGCCCCTGATAAGGCCATTACTCAGGAACAGTGTGAGG |
| | CCAGGGGCTGCTGCTATATCCCTGCCAAGCAGGGCCTGCAGGGGGCCCAGATGGGGCAGCCCTGGTGCTTCTTCCCCCCCAGCTACCCCTCTTATAAGCTGGAGAATCTGAGCAGCTCTGAAATGGGCTACACTGCCACTCTGAC |
| | CAGGACTACCCCCACCTTCTTCCCCAAGGATATTCTGACTCTGAGGCTGGATGTGATGATGGAGACTGAGAACAGGCTGCACTTCACCATCAAGGACCCTGCCAACAGGAGGTATGAGGTGCCCCTGGAAACTCCCAGGGTGCAC |
| | TCTAGGGCCCCCAGCCCCCTGTATTCTGTGGAGTTCTCTGAGGAGCCTTTTGGGGTCATTGTCCACAGGCAGCTGGATGGCAGGGTGCTGCTGAATACTACTGTGGCCCCTCTGTTCTTTGCTGACCAGTTCCTGCAGCTGAGCA |
| | CCAGCCTGCCTTCTCAGTACATTACTGGCCTGGCTGAGCATCTGAGCCCCCTGATGCTGAGCACCTCTTGGACCAGAATCACCCTGTGGAACAGGGACCTGGCTCCCACTCCTGGGGCCAACCTGTATGGCAGCCACCCCTTCTA |
| | CCTGGCCCTGGAGGATGGGGGCTCTGCCCATGGGGTGTTCCTGCTGAACAGCAATGCCATGGATGTGGTGCTGCAGCCCTCTCCTGCCCTGTCTTGGAGATCTACTGGGGGCATCCTGGATGTGTATATCTTCCTGGGGCCTGAG |
| | CCCAAGTCTGTGGTGCAGCAGTACCTGGATGTGGTGGGCTACCCCTTCATGCCCCCCTACTGGGGCCTGGGCTTCCACCTGTGCAGGTGGGGCTACTCTTCTACTGCTATCACCAGGCAGGTGGTGGAGAACATGACCAGGGCTC |
| | ACTTCCCTCTGGATGTGCAGTGGAATGACCTGGACTACATGGACTCTAGGAGAGACTTCACTTTTAATAAGGATGGGTTCAGGGACTTTCCTGCCATGGTGCAGGAGCTGCATCAGGGGGGCAGGAGATATATGATGATTGTGGA |
| | CCCTGCTATTTCTAGCTCTGGCCCTGCTGGCAGCTATAGGCCCTATGATGAGGGGCTGAGGAGGGGGGTGTTCATCACTAATGAGACTGGCCAGCCCCTGATTGGCAAGGTGTGGCCTGGCTCTACTGCCTTCCCTGATTTCACC |
| | AACCCCACTGCCCTGGCCTGGTGGGAGGATATGGTGGCTGAGTTTCATGACCAGGTGCCCTTTGATGGCATGTGGATTGACATGAATGAGCCCAGCAACTTTATCAGGGGCTCTGAAGATGGCTGCCCCAACAATGAGCTGGAGA |
| | ACCCCCCCTATGTGCCTGGGGTGGTGGGGGGCACCCTGCAGGCTGCCACCATCTGTGCCAGCAGCCACCAGTTCCTGAGCACCCACTATAACCTGCACAACCTGTATGGCCTGACTGAGGCCATTGCCTCTCACAGGGCCCTGGT |
| | GAAGGCTAGGGGGACTAGGCCCTTTGTGATCAGCAGGTCTACTTTTGCTGGCCATGGCAGGTATGCTGGGCACTGGACTGGGGATGTGTGGTCTAGCTGGGAGCAGCTGGCCAGCTCTGTGCCTGAGATCCTGCAGTTTAATCTG |
| | CTGGGGGTGCCCCTGGTGGGGGCTGATGTGTGTGGCTTCCTGGGCAATACCTCTGAGGAGCTGTGTGTGAGGTGGACTCAGCTGGGGGCTTTCTACCCCTTCATGAGAAACCACAACTCTCTGCTGAGCCTGCCCCAGGAGCCCT |
| | ATTCTTTTTCTGAGCCTGCCCAGCAGGCTATGAGGAAGGCCCTGACTCTGAGGTATGCCCTGCTGCCCCACCTGTATACCCTGTTCCATCAGGCCCATGTGGCTGGGGAGACTGTGGCCAGACCTCTGTTCCTGGAGTTCCCCAA |
| | GGATAGCTCTACTTGGACTGTGGACCACCAGCTGCTGTGGGGGGAGGCTCTGCTGATCACCCCTGTGCTGCAGGCTGGGAAGGCTGAGGTGACTGGCTATTTCCCCCTGGGCACCTGGTATGATCTGCAGACTGTGCCCATTGAG |
| | GCCCTGGGCTCTCTGCCCCCTCCCCCTGCTGCCCCCAGGGAGCCTGCCATCCACTCTGAGGGCCAGTGGGTGACCCTGCCTGCCCCTCTGGACACTATCAATGTGCACCTGAGGGCTGGCTACATCATCCCCCTGCAGGGCCCTG |
| | GCCTGACTACCACTGAGTCTAGGCAGCAGCCCATGGCCCTGGCTGTGGCTCTGACCAAGGGGGGGGAGGCCAGGGGGGAGCTGTTCTGGGATGATGGGGAGTCTCTGGAGGTGCTGGAGAGGGGGGCCTACACCCAGGTGATCTT |
| | CCTGGCTAGGAATAACACCATTGTCAATGAGCTGGTGAGGGTGACCTCTGAGGGGGCTGGCCTGCAGCTGCAGAAGGTGACTGTGCTGGGGGTGGCTACTGCCCCCCAGCAGGTGCTGAGCAATGGGGTGCCTGTGAGCAACTTC |
| | ACCTATAGCCCTGACACCAAGGTGCTGGACATCTGTGTGAGCCTGCTGATGGGGGAGCAGTTCCTGGTGAGCTGGTGCTAA |
| 40 | >5′ UTR\|SERPINA7 |
| | CAGGTGGATTCTTGGGCATTTGCTTAAGCTTGGTACCGCCACC |
| 41 | > 5′ UTR\|24BP |
| | CAGGTGGATTCTTGGGCATTTGCT |
| 42 | >FL-POLYA |
| | ATCTGTGGCTTCTAGCTGCCCGGGTGGCATCCCTGTGACCCCTCCCCAGTGCCTCTCCTGGCCCTGGAAGTTGCCACTCCAGTGCCCACCAGCCTTGTCCTAATAAAATTAAGTTGCATCATTTTGTCTGACTAGGTGTCCTTCT |
| | ATAATATTATGGGGTGGAGGGGGGTGGTATGGAGCAAGGGGCAAGTTGGG |
| 43 | CTGTGGCTTCTAGCTGCCCGGGTGGCATCCCTGTGACCCCTCCCCAGTGCCTCTCCTGGCCCTGGAAGTTGCCACTCCAGTGCCCACCAGCCTTGTCCTAATAAAATTAAGTTGCATCATTTTGTCTGACTAGGTGTCCTTCTAT |
| | AATATTATGGGGTGGAGGGGGGTGGTATGGAGCAAGGGGCAAGTTGGG |
| 44 | >FL-POLYA plus AATAA signal |
| | CTGTGGCTTCTAGCTGCCCGGGTGGCATCCCTGTGACCCCTCCCCAGTGCCTCTCCTGGCCCTGGAAGTTGCCACTCCAGTGCCCACCAGCCTTGTCCTAATAAAATTAAGTTGCATCATTTTGTCTGACTAGGTGTCCTTCTAT |
| | AATATTATGGGGTGGAGGGGGGTGGTATGGAGCAAGGGGCAAGTTGGG |
| 45 | >REV RNA POLII_TERMINATOR |
| | AGAGAAATGTTCTGGCACCTGCACTTGCACTGGGGACAGCCTATTTTGCTAGTTTGTTTTGTTTCGTTTTGTTTTGATGGAGAGCGTATGTT |
| 46 | >Stuffer DNA with 48 bp synthetic polyA |
| | GTCCTTCAACGTCGAACTGTAGCACTCTGGATAGCTAGCTAGCGTTTGTAGATAGTTTCAAGGATAGCAACCAACAACACGATAGTTTAGAAATTGTTCTAGATCTTCTGCTACTACTCGAGAGATAGTTGACTTCTAGAAGACT |
| | CTGGATAGAGAGCTTCCTGTGGTTTCTATTTCAACACTTCGAGCGATTTAGATAGAGGCGTCAATTGAGAACAAAAACTGGACACCTGTTTCTAGAACACTAGTGCTAGTCTTCTCAAGGCTTGATACGTCCTGCCACTCACACA |
| | CAAAAAACCAACACACAGATTAATGAAAATAAAGATCTTTTATTAGAGAGCGAGTGGATAGTTTAGAGTCGCTTGAGACTTATCCTGTGTCCAACTAGCCAAGAGTAGAGAGAGAGTGACTAGGTGGATCGAGAGATCGTCGTCT |
| | ATTTGACTGCTAGCCTGTGTTCTCGTCGTAGTCCAAATCTTCAATACTCTGGCGACTAGTAACTTGAATCTGTGTATCTAACACGACAAGGCTACTCCTTTGAGTTTCTAGTGATAAGTGCGACTGGATCTAGCTAGTTGAGTTT |
| | CTAGAAACTACGTCCAAAGCTACTAGCACTA |
| 47 | >synthetic polyA |
| | CACACAAAAAACCAACACACAGATTAATGAAAATAAAGATCTTTTATT |
| 48 | >3′UTR |
| | AGCCGGGCGGAGTGTGTTAGTCTCTCCAGAGGGAGGCTGGTTCCCCAGGGAAGCAGAGCCTGTGTGCGGGCAGCAGCTGTGTGCGGGCCTGGGGGTTG |
| 49 | >3′UTR |
| | CCGGGCGGAGTGTGTTAGTCTCTCCAGAGGGAGGCTGGTTCCCCAGGGAAGCAGAGCCTGTGTGCGGGCAGCAGCTGTGTGCGGGCCTGGGGGTTG |
| 50 | >GAA 3′UTR |
| | CCGGGCGGAGTGTGTTAGTCTCTCCAGAGGGAGGCTGGTTCCCCAGGGAAGCAGAGCCTGTGTGCGGGCAGCAGCTGTGTGCGGGCCTGGGGGTTGTTAAGTGCAATTATTTTTAATAAAAGGGGCATTTGGAAAAAAAAAAAAA |
| | AGGTAGCAGTCGACAGATGA |
| 51 | >endogenous wtGAA signal peptide |
| | ATGGGAGTGAGGCACCCCCCCTGCTCCCATAGACTCCTGGCTGTCTGTGCCCTGGTGTCCTTGGCAACAGCTGCACTC |
| 52 | >N-terminal deletion of GAA nucleic acid sequence |
| | ATGGGAGTGAGGCACCCCCCCTGCTCCCATAGACTCCTGGCTGTCTGTGCCCTGGTGTCCTTGGCAACAGCTGCACTCCTGGGGCACATCCTACTCCATGATTTCCTGCTGGTTCCCAGAGAGCTGAGTGGCTCCTCCCCAGTCC |
| | TGGAGGAGACTCACCCAGCTCAC |
| 53 | >nucleotide of codon optimized GAA signal peptide |
| | ATGGGAGTGAGGCACCCGCCCTGCTCCCACCGGCTCCTGGCCGTCTGCGCCCTCGTGTCCTTGGCAACCGCTGCACTCCTG |
| 54 | >nucleotide of 201 signal peptide |
| | ATGGAGTTCGGGCTGAGCTGGGTCTTTCTGGTGGCCCTGCTGAAGGGAGTCCAGTGCGAG |
| 55 | >nucleotide of wt IL2 signal peptide |
| | ATGTACCGTATGCAGCTGCTGTCCTGCATCGCACTGTCACTGGCACTGGTTACCAACTCA |
| 56 | >nucleotide of mut IL2 signal peptide |
| | ATGTACCGTATGCAGCTGCTGCTGCTGATCGCACTGTCACTGGCACTGGTTACCAACTCA |
| 57 | >nucleotide of 2AM signal peptide |
| | ATGGGGAAGAACAAACTCCTTCATCCAAGTCTGGTTCTTCTCCTCTTGGTCCTCCTGCCCACAGATGCC |
| 58 | >nucleotide of PZP signal peptide |
| | ATGAGAAAAGACAGACTTCTTCATTTATGTCTTGTGCTACTTCTTATCCTGCTTTCTGCCAGTGACTCAAACTCT |
| 59 | >endogenous GAA signal peptide |
| | MGVRHPPCSHRLLAVCALVSLATAALL |
| 60 | >201 signal peptide |
| | MEFGLSWVFLVALLKGVQCE |
| 61 | >wt IL2 signal peptide |
| | MYRMQLLSCIALSLALVTNS |
| 62 | >mut IL2 signal peptide |
| | MYRMQLLLLIALSLALVTNS |
| 63 | >A2M signal peptide |
| | MGKNKLLHPSLVLLLLVLLPTDA |
| 64 | >PZP signal peptide |
| | MRKDRLLHLCLVLLLILLSASDSNS |
| 65 | >artificial signal peptide |
| | MWWRLWWLLLLLLLLWPMVWA |
| 66 | > cathpetsin L signal peptide |
| | LLLLAVLCLGT |
| 67 | >nucleotide of hAAT signal peptide |
| | ATGCCGTCTTCTGTCTCGTGGGGCATCCTCCTGCTGGCAGGCCTGTGCTGCCTGGTCCCTGTCTCCCTGGCT |
| 68 | > FIBrat Signal Peptide |
| | MLRGPGPGRLLLLAVLCLGTSVRCTETGKSKR |
| 69 | > FIBrat Signal Peptide |
| | MLRGPGPGRLLLLAVLCLGTSVRCTETGKSKRLALQIV |
| 70 | >FN1 human signal peptide |
| | MLRGPGPGLLLLAVQCLGTAVPSTGA |
| 71 | >FN1 x. leavis signal peptide |
| | MRRGALTGLLLVLCLSVVLRAAPSATSKKRR |
| 72 | >hIGF2 signal peptide |
| | ATGGGAATCCCAATGGGGAAGTCGATGCTGGTGCTTCTCACCTTCTTGGCCTTCGCCTCGTGCTGCATTGCT |
| 73 | >FN1 rat signal peptide |
| | ATGCTCAGGGGTCCGGGACCCGGGCGGCTGCTGCTGCTAGCAGTCCTGTGCCTGGGGACATCGGTGCGCTGCACCGAAACCGGGAAGAGCAAGAGG |
| 74 | >FN1 rat signal peptide |
| | ATG CTC AGG GGT CCG GGA CCC GGG CGG CTG CTG CTG CTA GCA GTC CTG TGC CTG GGG ACA TCG GTG CGC TGC ACC GAA ACC GGG AAG AGC AAG AGG CAG GCT CAG CAA |
| | ATC GTG-3′. |
| 75 | >FN1 human signal peptide |
| | ATGCTTAGGGGTCCGGGGCCCGGGCTGCTGCTGCTGGCCGTCCAGTGCCTGGGGACAGCGGTGCCCTCCACGGGAGCC |
| 76 | >FN1 x. leavis signal peptide |
| | ATG CGC CGG GGG GCC CTG ACC GGG CTG CTC CTG GTC CTG TGC CTG AGT GTT GTG CTA CGT GCA GCC CCC TCT GCA ACA AGC AAG AAG CGC AGG |
| 77 | > preprocathepsin L signal peptide (rat) |
| | MTPLLLLAVLCLGTALA |
| 78 | >prepro-alpha 2 type collagen signal peptide (homo sapiens) |
| | MLSFVDTRTLLLLAVTLCLATC |
| 79 | >SEQ 79 CTGCGCGCTCGCTCGCTCACTGAGGCCGCCCGGGCAAAGCCCGGGCGTCGGGCGACCTTTGGTCGCCCGGCCTCAGTGAGCGAGCGAGCGCGCAGAGAGGGAGTGGCCAACTCCATCACTAGGGGTTCCT |
| 80 | >SEQ 80 AGGAACCCCTAGTGATGGAGTTGGCCACTCCCTCTCTGCGCGCTCGCTCGCTCACTGAGGCCGGGCGACCAAAGGTCGCCCGACGCCCGGGCTTTGCCCGGGCGGCCTCAGTGAGCGAGCGAGCGCGCAG |
| 81 | >SEQ 81 CTGCGCGCTCGCTCGCTCACTGAGGCCGCCCGGGCAAAGCCCGGGCGTCGGGCGACCTTTGGTCGCCCGGCCTCAGTGAGCGAGCGAGCGCGCAGAGAGGGAGTGGCCAACTCCATCACTAGGG |
| 82 | >SEQ 82 |
| | AGGAACCCCTAGTGATGGAGTTGGCCACTCCCTCTCTGCGCGCTCGCTCGCTCACTGAGGCCGGGCGACCAAAGGTCGCCCGACGCCCGGGCTTTGCCCGGGCGGCCTCAGTGAGCGAGCGAGCGCGCAG |
| 83 | > MH-257 ITR |
| | AGGAACCCCTAGTGATGGAGTTGGCCACTCCCTCTCTGCGCGCTCGCTCGCTCACTGAGGCAATTTGATAAAAATCGTCAAATTATAAACAGGCTTTGCCTGTTTAGCCTCAGTGAGCGAGCGAGCGCGCAGAGAGGGAGTGGCC |
| | AACTCCATCACTAGGGGTTCCT |
| 84 | >MH-258 ITR |
| | AGGAACCCCTAGTGATGGAGTTGGCCACTCCCTCTCTGCGCGCTCGCTCGCTCACTGAGGGATAAAAATCCAGGCTTTGCCTGCCTCAGTGAGCGAGCGAGCGCGCAGAGAGGGAGTGGCCAACTCCATCACTAGGGGTTCCT |
| 85 | >MH Delta |
| | 258AGGAACCCCTAGTGATGGAGTTGGCCACTCCCTCTCTGCGCGCTCGCTCGCTCACTGAGGGATAAAAATCCAGGCTTTGCCTGCCTCAGTGAGCGAGCGAGCGCGCAGAGAGGGAGTGGCCAACTCCATCACTAGGGGTTCC |
| | T |
| 100 | >MH Telomere-1 ITR |
| | AGGAACCCCTAGTGATGGAGTTGGCCACTCCCTCTCTGGGATTGGGATTGCGCGCTCGCTCGCGGGATTGGGATTGGGATTGGGATTGGGATTGGGATTGATAAAAATCAATCCCAATCCCAATCCCAATCCCAATCCCAATCCC |
| | GCGAGCGAGCGCGCAATCCCAATCCCAGAGAGGGAGTGGCCAACTCCATCACTAGGGGTTCCT |
| 101 | >MH Telomere-2 ITR |
| | AGGAACCCCTAGTGATGGAGTTGGCCACTCCCTCTCTGCGCGCTCGCTCGCTCGGGATTGGGATTGGGATTGGGATTGGGATTGGGATTGATAAAAATCAATCCCAATCCCAATCCCAATCCCAATCCCAATCCCGCGAGCGAGC |
| | GCGCAGGAGAGGGAGTGGCCAACTCCATCACTAGGGGTTCCTAAGCTTATTATA |
| 102 | >MH PolII 258 ITR |
| | AGGAACCCCTAGTGATGGAGTTGGCCACTCCCTCTCTGCGCGCTCGCTCGCTCACTGAGGGCGCCTATAAAGATAAAAATCCAGGCTTTGCCTGCCTCAGTTAGCGAGCGAGCGCGCAGAGAGGGAGTGGCCAACTCCATCACTA |
| | GGGGTTCCT |
| 103 | >MH 258 Delta D conservative |
| | CTAGTGATGGAGTTGGCCACTCCCTCTCTGCGCGCTCGCTCGCTCACTGAGGGATAAAAATCCAGGCTTTGCCTGCCTCAGTGAGCGAGCGAGCGCGCAGAGAGGGAGTGGCCAACTCCATCACTAG |
| 104 | >5′ AAV2-ITR |
| | TTGGCCACTCCCTCTCTGCGCGCTCGCTCGCTCACTGAGGCCGGGCGACCAAAGGTCGCCCGACGCCCGGGCTTTGCCCGGGCGGCCTCAGTGAGCGAGCGAGCGCGCAGAGAGGGAGTGGCCAACTCCATCACTAGGGGTTCCT |
| 105 | >3′ AAV2 ITR |
| | AGGAACCCCTAGTGATGGAGTTGGCCACTCCCTCTCTGCGCGCTCGCTCGCTCACTGAGGCCGGGCGACCAAAGGTCGCCCGACGCCCGGGCTTTGCCCGGGCGGCCTCAGTGAGCGAGCGAGCGCGCAGAGAGGGAGTGGCCAA |
| 448 | > ITR (145 bp) |
| | AGGAACCCCTAGTGATGGAGTTGGCCACTCCCTCTCTGCGCGCTCGCTCGCTCACTGAGGCCGGGCGACCAAAGGTCGCCCGACGCCCGGGCTTTGCCCGGGCGGCCTCAGTGAGCGAGCGAGCGCGCAGAGAGGGAGTGGCCAA |
| 449 | > ITR (lacking 9 bp) |
| | AGGAACCCCTAGTGATGGAGTTGGCCACTCCCTCTCTGCGCGCTCGCTCGCTCACTGAGGCCGGGCGACCAAAGGTCGCCCGACGCCCGGGCTTTGCCCGGGCGGCCTCAGTGAGCGAGCGAGCGCGCAGAGAGGG |
| 450 | >CRE0052 |
| | GGGCATATAAAACAGGGGCAAGGCACAGACTCATAGCAGAGCAATCACCACCAAGCCTGGAATAACTGCAGCCACC |
| 451 | > pAAV-LSPhGAA \|Actus101 plasmid |
| | CTGCGCGCTCGCTCGCTCACTGAGGCCGCCCGGGCAAAGCCCGGGCGTCGGGCGACCTTTGGTCGCCCGGCCTCAGTGAGCGAGCGAGCGCGCAGAGAGGGAGTGGCCAACTCCATCACTAGGGGTTCCTGGAGGGGTGGAGTCG |
| | TGACGTGAATTACGTCATAGGGTTAGGGAGGTCGGCCGCTCTAGGAGTTAATTTTTAAAAAGCAGTCAAAAGTCCAAGTGCCCTTGCGAGCATTTACTCTCTCTGTTTGCTCTGGTTAATAATCTCAGGAGCACAAACATTCCTT |
| | ACTAGTTCTAGGAGTTAATTTTTAAAAAGCAGTCAAAAGTCCAAGTGCCCTTGCGAGCATTTACTCTCTCTGTTTGCTCTGGTTAATAATCTCAGGAGCACAAACATTCCTTACTAGTTCTAGAGCGGCCGCCAGTGTGCTGGAA |
| | TTCGGCTTTTTTAGGGCTGGAAGCTACCTTTGACATCATTTCCTCTGCGAATGCATGTATAATTTCTACAGAACCTATTAGAAAGGATCACCCAGCCTCTGCTTTTGTACAACTTTCCCTTAAAAAACTGCCAATCCCACTGCTG |
| | TTTGGCCCAATAGTGAGAACTTTTTCCTGCTGCCTCTTGGTGCTTTTGCCTATGGCCCCTATTCTGCCTGCTGAAGACACTCTTGCCAGCATGGACTTAAACCCCTCCAGCTCTGACAATCCTCTTTCTCTTTTGTTTTACATGA |
| | AGGGTCTGGCAGCCAAAGCAATCACTCAAAGTTCAAACCTTATCATTTTTTGCTTTGTTCCTCTTGGCCTTGGTTTTGTACATCAGCTTTGAAAATACCATCCCAGGGTTAATGCTGGGGTTAATTTATAACTGAGAGTGCTCTA |
| | GTTCTGCAATACAGGACATGCTATAAAAATGGAAAGATGTTGCTTTCTGAGAGATCAGCTTACATGTGGTACCGAGCTCGGATCCACTAGTAACGGCCGCCAGTGTGCTGGAATTCGGCTTTCCAGGCCATCTCCAACCATGGGA |
| | GTGAGGCACCCGCCCTGCTCCCACCGGCTCCTGGCCGTCTGCGCCCTCGTGTCCTTGGCAACCGCTGCACTCCTGGGGCACATCCTACTCCATGATTTCCTGCTGGTTCCCCGAGAGCTGAGTGGCTCCTCCCCAGTCCTGGAGG |
| | AGACTCACCCAGCTCACCAGCAGGGAGCCAGCAGACCAGGGCCCCGGGATGCCCAGGCACACCCCGGCCGTCCCAGAGCAGTGCCCACACAGTGCGACGTCCCCCCCAACAGCCGCTTCGATTGCGCCCCTGACAAGGCCATCAC |
| | CCAGGAACAGTGCGAGGCCCGCGGCTGCTGCTACATCCCTGCAAAGCAGGGGCTGCAGGGAGCCCAGATGGGGCAGCCCTGGTGCTTCTTCCCACCCAGCTACCCCAGCTACAAGCTGGAGAACCTGAGCTCCTCTGAAATGGGC |
| | TACACGGCCACCCTGACCCGTACCACCCCCACCTTCTTCCCCAAGGACATCCTGACCCTGCGGCTGGACGTGATGATGGAGACTGAGAACCGCCTCCACTTCACGATCAAAGATCCAGCTAACAGGCGCTACGAGGTGCCCTTGG |
| | AGACCCCGCGTGTCCACAGCCGGGCACCGTCCCCACTCTACAGCGTGGAGTTCTCCGAGGAGCCCTTCGGGGTGATCGTGCACCGGCAGCTGGACGGCCGCGTGCTGCTGAACACGACGGTGGCGCCCCTGTTCTTTGCGGACCA |
| | GTTCCTTCAGCTGTCCACCTCGCTGCCCTCGCAGTATATCACAGGCCTCGCCGAGCACCTCAGTCCCCTGATGCTCAGCACCAGCTGGACCAGGATCACCCTGTGGAACCGGGACCTTGCGCCCACGCCCGGTGCGAACCTCTAC |
| | GGGTCTCACCCTTTCTACCTGGCGCTGGAGGACGGCGGGTCGGCACACGGGGTGTTCCTGCTAAACAGCAATGCCATGGATGTGGTCCTGCAGCCGAGCCCTGCCCTTAGCTGGAGGTCGACAGGTGGGATCCTGGATGTCTACA |
| | TCTTCCTGGGCCCAGAGCCCAAGAGCGTGGTGCAGCAGTACCTGGACGTTGTGGGATACCCGTTCATGCCGCCATACTGGGGCCTGGGCTTCCACCTGTGCCGCTGGGGCTACTCCTCCACCGCTATCACCCGCCAGGTGGTGGA |
| | GAACATGACCAGGGCCCACTTCCCCCTGGACGTCCAATGGAACGACCTGGACTACATGGACTCCCGGAGGGACTTCACGTTCAACAAGGATGGCTTCCGGGACTTCCCGGCCATGGTGCAGGAGCTGCACCAGGGCGGCCGGCGC |
| | TACATGATGATCGTGGATCCTGCCATCAGCAGCTCGGGCCCTGCCGGGAGCTACAGGCCCTACGACGAGGGTCTGCGGAGGGGGGTTTTCATCACCAACGAGACCGGCCAGCCGCTGATTGGGAAGGTATGGCCCGGGTCCACTG |
| | CCTTCCCCGACTTCACCAACCCCACAGCCCTGGCCTGGTGGGAGGACATGGTGGCTGAGTTCCATGACCAGGTGCCCTTCGACGGCATGTGGATTGACATGAACGAGCCTTCCAACTTCATCAGAGGCTCTGAGGACGGCTGCCC |
| | CAACAATGAGCTGGAGAACCCACCCTACGTGCCTGGGGTGGTTGGGGGGACCCTCCAGGCGGCCACCATCTGTGCCTCCAGCCACCAGTTTCTCTCCACACACTACAACCTGCACAACCTCTACGGCCTGACCGAAGCCATCGCC |
| | TCCCACAGGGCGCTGGTGAAGGCTCGGGGGACACGCCCATTTGTGATCTCCCGCTCGACCTTTGCTGGCCACGGCCGATACGCCGGCCACTGGACGGGGGACGTGTGGAGCTCCTGGGAGCAGCTCGCCTCCTCCGTGCCAGAAA |
| | TCCTGCAGTTTAACCTGCTGGGGGTGCCTCTGGTCGGGGCCGACGTCTGCGGCTTCCTGGGCAACACCTCAGAGGAGCTGTGTGTGCGCTGGACCCAGCTGGGGGCCTTCTACCCCTTCATGCGGAACCACAACAGCCTGCTCAG |
| | TCTGCCCCAGGAGCCGTACAGCTTCAGCGAGCCGGCCCAGCAGGCCATGAGGAAGGCCCTCACCCTGCGCTACGCACTCCTCCCCCACCTCTACACACTGTTCCACCAGGCCCACGTCGCGGGGGAGACCGTGGCCCGGCCCCTC |
| | TTCCTGGAGTTCCCCAAGGACTCTAGCACCTGGACTGTGGACCACCAGCTCCTGTGGGGGGAGGCCCTGCTCATCACCCCAGTGCTCCAGGCCGGGAAGGCCGAAGTGACTGGCTACTTCCCCTTGGGCACATGGTACGACCTGC |
| | AGACGGTGCCAATAGAGGCCCTTGGCAGCCTCCCACCCCCACCTGCAGCTCCCCGTGAGCCAGCCATCCACAGCGAGGGGCAGTGGGTGACGCTGCCGGCCCCCCTGGACACCATCAACGTCCACCTCCGGGCTGGGTACATCAT |
| | CCCCCTGCAGGGCCCTGGCCTCACAACCACAGAGTCCCGCCAGCAGCCCATGGCCCTGGCTGTGGCCCTGACCAAGGGTGGAGAGGCCCGAGGGGAGCTGTTCTGGGACGATGGAGAGAGCCTGGAAGTGCTGGAGCGAGGGGCC |
| | TACACACAGGTCATCTTCCTGGCCAGGAATAACACGATCGTGAATGAGCTGGTACGTGTGACCAGTGAGGGAGCTGGCCTGCAGCTGCAGAAGGTGACTGTCCTGGGCGTGGCCACGGCGCCCCAGCAGGTCCTCTCCAACGGTG |
| | TCCCTGTCTCCAACTTCACCTACAGCCCCGACACCAAGGTCCTGGACATCTGTGTCTCGCTGTTGATGGGAGAGCAGTTTCTCGTCAGCTGGTGTTAGCCGGGCGGAGTGTGTTAGTCTCTCCAGAGGGAGGCTGGTTCCCCAGG |
| | GAAGCAGAGCCTGTGTGCGGGCAGCAGCTGTGTGCGGGCCTGGGGGTTGTTAAGTGCAATTATTTTTAATAAAAGGGGCATTTGGAAAAAAAAAAAAAAGGTAGCAGTCGACAGATGAATTCTGCAGATCTGTGGCTTCTAGCTG |
| | CCCGGGTGGCATCCCTGTGACCCCTCCCCAGTGCCTCTCCTGGCCCTGGAAGTTGCCACTCCAGTGCCCACCAGCCTTGTCCTAATAAAATTAAGTTGCATCATTTTGTCTGACTAGGTGTCCTTCTATAATATTATGGGGTGGA |
| | GGGGGGTGGTATGGAGCAAGGGGCAAGTTGGGAAGACAACCTGAGTTGTTGGGATTCCAGGCATCGAGTAGATAAGTAGCATGGCGGGTTAATCATTAACTACAAGGAACCCCTAGTGATGGAGTTGGCCACTCCCTCTCTGCGC |
| | GCTCGCTCGCTCACTGAGGCCGGGCGACCAAAGGTCGCCCGACGCCCGGGCTTTGCCCGGGCGGCCTCAGTGAGCGAGCGAGCGCGCAG |
| 97 | >LSP\|LSP_ACTUS_ 101 |
| | TCTAGGAGTTAATTTTTAAAAAGCAGTCAAAAGTCCAAGTGCCCTTGCGAGCATTTACTCTCTCTGTTTGCTCTGGTTAATAATCTCAGGAGCACAAACATTCCTTACTAGTTCTAGGAGTTAATTTTTAAAAAGCAGTCAAAAG |
| | TCCAAGTGCCCTTGCGAGCATTTACTCTCTCTGTTTGCTCTGGTTAATAATCTCAGGAGCACAAACATTCCTTACTAGTTCTAGAGCGGCCGCCAGTGTGCTGGAATTCGGCTTTTTTAGGGCTGGAAGCTACCTTTGACATCAT |
| | TTCCTCTGCGAATGCATGTATAATTTCTACAGAACCTATTAGAAAGGATCACCCAGCCTCTGCTTTTGTACAACTTTCCCTTAAAAAACTGCCAATCCCACTGCTGTTTGGCCCAATAGTGAGAACTTTTTCCTGCTGCCTCTTG |
| | GTGCTTTTGCCTATGGCCCCTATTCTGCCTGCTGAAGACACTCTTGCCAGCATGGACTTAAACCCCTCCAGCTCTGACAATCCTCTTTCTCTTTTGTTTTACATGAAGGGTCTGGCAGCCAAAGCAATCACTCAAAGTTCAAACC |
| | TTATCATTTTTTGCTTTGTTCCTCTTGGCCTTGGTTTTGTACATCAGCTTTGAAAATACCATCCCAGGGTTAATGCTGGGGTTAATTTATAACTGAGAGTGCTCTAGTTCTGCAATACAGGACATGCTATAAAAATGGAAAGATG |
| | TTGCTTTCTGAGAGAT |
| 98 | >M2 LSP |
| | TCTAGGAGTTAATTTTTAAAAAGCAGTCAAAAGTCCAAGTGCCCTTGCGAGCATTTACTCTCTCTGTTTGCTCTGGTTAATAATCTCAGGAGCACAAACATTCCTTACTAGTTCTAGGAGTTAATTTTTAAAAAGCAGTCAAAAG |
| | TCCAAGTGCCCTTGCGAGCATTTACTCTCTCTGTTTGCTCTGGTTAATAATCTCAGGAGCACAAACATTCCTTACTAGTTCTAGAGCGGCCGCCAGTGTGCTGGAATTCGGCTTTTTTAGGGCTGGAAGCTACCTTTGACATCAT |
| | TTCCTCTGCGAATGCATGTATAATTTCTACAGAACCTATTAGAAAGGATCACCCAGCCTCTGCTTTTGTACAACTTTCCCTTAAAAAACTGCCAATCCCACTGCTGTTTGGCCCAATAGTGAGAACTTTTTCCTGCTGCCTCTTG |
| | GTGCTTTTGCCTATGGCCCCTATTCTGCCTGCTGAAGACACTCTTGCCAGCATGGACTTAAACCCCTCCAGCTCTGACAATCCTCTTTCTCTTTTGTTTTACATGAAGGGTCTGGCAGCCAAAGCAATCACTCAAAGTTCAAACC |
| | TTATCATTTTTTGCTTTGTTCCTCTTGGCCTTGGTTTTGTACATCAGCTGTGAAAATACCATCCCAGGGTATTTATAGCTGGGGTTAATTTATAACTGAGAGTGCTCTAGTTCTGCAATACAGGACATGCTATAAAAATGGAAAG |
| | ATGTTGCTTTCTGAGAGATCAGCTTACATGTG (SEQ ID NO: 437) |
| 99 | >M3 LSP |
| | TCTAGGAGTTAATTTTTAAAAAGCAGTCAAAAGTCCAAGTGCCCTTGCGAGCATTTACTCTCTCTGTTTGCTCTGGTTAATAATCTCAGGAGCACAAACATTCCTTACTAGTTCTAGGAGTTAATTTTTAAAAAGCAGTCAAAAG |
| | TCCAAGTGCCCTTGCGAGCATTTACTCTCTCTGTTTGCTCTGGTTAATAATCTCAGGAGCACAAACATTCCTTACTAGTTCTAGAGCGGCCGCCAGTGTGCTGGAATTCGGCTTTTTTAGGGCTGGAAGCTACCTTTGACATCAT |
| | TTCCTCTGCGAATGCATGTATAATTTCTACAGAACCTATTAGAAAGGATCACCCAGCCTCTGCTTTTGTACAACTTTCCCTTAAAAAACTGCCAATCCCACTGCTGTTTGGCCCAATAGTGAGAACTTTTTCCTGCTGCCTCTTG |
| | GTGCTTTTGCCTATGGCCCCTATTCTGCCTGCTGAAGACACTCTTGCCAGCATGGACTTAAACCCCTCCAGCTCTGACAATCCTCTTTCTCTTTTGTTTTACATGAAGGGTCTGGCAGCCAAAGCAATCACTCAAAGTTCAAACC |
| | TTATCATTTTTTGCTTTGTTCCTCTTGGCCTTGGTTTTGTACATCAGCTTTGAAAATACCATCCCAGGGTTAATGCTGGGGTTAATTTATAACTGAGAGTGCTCTAGTTCTGCAATACAGGACATGCTATAAAAATGGAAAGATG |
| | TTGCTTTCTGAGAGATCAGCTTACATGTG |
| 86 | > CRM_SP0412 |
| | AGGTTAATTTTTAAAAAGCAGTCAAAAGTCCAAGTGGCCCTTGGCAGCATTTACTCTCTCTGTTTGCTCTGGTTAATAATCTCAGGAGCACAAACATTCCCCCTGTTCAAACATGTCCTAATACTCTGTCTCTGCAAGGGTCATC |
| | AGTAGTTTTCCATCTTACTCAACATCCTCCCAGTG |
| 87 | > CRM_SP0239 |
| | CAGGCTTTCACTTTCTCGCCAACTTACAAGGCCTTTCTGTGTAAACAATACCTGAACCTTTACCCCGTTGCCCGGCAACGGCCAGGTCTGTGCCAAGTGTTTGAGGTTAATTTTTAAAAAGCAGTCAAAAGTCCAAGTGGCCCTT |
| | GGCAGCATTTACTCTCTCTGTTTGCTCTGGTTAATAATCTCAGGAGCACAAACATTCCGGCCCGGGAGGCGCCCTTTGGACCTTTTGCAATCCTGGCGCACTGAACCCTTGACCCCTGCCCTGCAGCCCCCGCAGCTTGCTGTTT |
| | GCCCACTCTATTTGCCCAGCCCCAGCCCTGGAGAGTCCTTTAGCAGGGCAAAGTGCAACATAGGCAGACCTTAAGGGATGACTCAGTAACAGATAAGCTTTGTGTGCCTGCA |
| 88 | > CRM_SP0265 (CRM_LVR_131_A1) |
| | AGGTTAATTTTTAAAAAGCAGTCAAAAGTCCAAGTGGCCCTTGGCAGCATTTACTCTCTCTGTTTGCTCTGGTTAATAATCTCAGGAGCACAAACATTCCTGTACCGGCCCGGGAGGCGCCCTTTGGACCTTTTGCAATCCTGGC |
| | GCACTGAACCCTTGACCCCTGCCCTGCAGCCCCCGCAGCTTGCTGTTTGCCCACTCTATTTGCCCAGCCCCAGCCCTGGAGAGTCCTTTAGCAGGGCAAAGTGCAACATAGGCAGACCTTAAGGGATGACTCAGTAACAGATAAG |
| | CTTTGTGTGCCTGCA |
| 89 | > CRM_SP0240 |
| | CAGGCTTTCACTTTCTCGCCAACTTACAAGGCCTTTCTGTGTAAACAATACCTGAACCTTTACCCCGTTGCCCGGCAACGGCCAGGTCTGTGCCAAGTGTTTG |
| 90 | > CRM_SP0246 |
| | AGGTTAATTTTTAAAAAGCAGTCAAAAGTCCAAGTGGCCCTTGGCAGCATTTACTCTCTCTGTTTGCTCTGGTTAATAATCTCAGGAGCACAAACATTCCGGCCCGGGAGGCGCCCTTTGGACCTTTTGCAATCCTGGCGCACTG |
| | AACCCTTGACCCCTGCCCTGCAGCCCCCGCAGCTTGCTGTTTGCCCACTCTATTTGCCCAGCCCCAG |
| 91 | > SP0412 |
| | AGGTTAATTTTTAAAAAGCAGTCAAAAGTCCAAGTGGCCCTTGGCAGCATTTACTCTCTCTGTTTGCTCTGGTTAATAATCTCAGGAGCACAAACATTCCCCCTGTTCAAACATGTCCTAATACTCTGTCTCTGCAAGGGTCATC |
| | AGTAGTTTTCCATCTTACTCAACATCCTCCCAGTGAGTCATATGTTTGCTCACTGAAGGTTACTAGTTAACAGGCATCCCTTAAACAGGATATAAAAGGACTTCAGCAGGACTGCTCGAAACATCCCACTCAGCCACC |
| 92 | > SP0422 |
| | AGGTTAATTTTTAAAAAGCAGTCAAAAGTCCAAGTGGCCCTTGGCAGCATTTACTCTCTCTGTTTGCTCTGGTTAATAATCTCAGGAGCACAAACATTCCCCCTGTTCAAACATGTCCTAATACTCTGTCTCTGCAAGGGTCATC |
| | AGTAGTTTTCCATCTTACTCAACATCCTCCCAGTGAGTCATATGTTTGCTCACTGAAGGTTACTAGTTAACAGGCATCCCTTAAACAGGATATAAAAGGACTTCAGCAGGACTGCTCGAAACATCCCACTTCAGATCGCCTGGAG |
| | ACGCCATCCACGCTGTTTTGACCTCCATAGAAGACACCGGGACCGATCCAGCCTCCGCGGCCGGGAACGGTGCATTGGAACGCGGATTCCCCGTGCCAAGAGTGACGTAAGTACCGCCTATAGACTCTATAGGCACACCCCTTTG |
| | GCTCTTATGCATGAACGGTGGAGGGCAGTGTAGTCTGAGCAGTACTCGTTGCTGCCGCGCGCGCCACCAGACATAATAGCTGACAGACTAACAGACTGTTCCTTTCCATGGGTCTTTTCTGCAGGCCACC |
| 93 | > SP0239 |
| | CAGGCTTTCACTTTCTCGCCAACTTACAAGGCCTTTCTGTGTAAACAATACCTGAACCTTTACCCCGTTGCCCGGCAACGGCCAGGTCTGTGCCAAGTGTTTGAGGTTAATTTTTAAAAAGCAGTCAAAAGTCCAAGTGGCCCTT |
| | GGCAGCATTTACTCTCTCTGTTTGCTCTGGTTAATAATCTCAGGAGCACAAACATTCCGGCCCGGGAGGCGCCCTTTGGACCTTTTGCAATCCTGGCGCACTGAACCCTTGACCCCTGCCCTGCAGCCCCCGCAGCTTGCTGTTT |
| | GCCCACTCTATTTGCCCAGCCCCAGCCCTGGAGAGTCCTTTAGCAGGGCAAAGTGCAACATAGGCAGACCTTAAGGGATGACTCAGTAACAGATAAGCTTTGTGTGCCTGCAGGGCATATAAAACAGGGGCAAGGCACAGACTCA |
| | TAGCAGAGCAATCACCACCAAGCCTGGAATAACTGCAGCCACC |
| 94 | > SP0265 (LVR_SP131_A1) |
| | AGGTTAATTTTTAAAAAGCAGTCAAAAGTCCAAGTGGCCCTTGGCAGCATTTACTCTCTCTGTTTGCTCTGGTTAATAATCTCAGGAGCACAAACATTCCTGTACCGGCCCGGGAGGCGCCCTTTGGACCTTTTGCAATCCTGGC |
| | GCACTGAACCCTTGACCCCTGCCCTGCAGCCCCCGCAGCTTGCTGTTTGCCCACTCTATTTGCCCAGCCCCAGCCCTGGAGAGTCCTTTAGCAGGGCAAAGTGCAACATAGGCAGACCTTAAGGGATGACTCAGTAACAGATAAG |
| | CTTTGTGTGCCTGCAGGGCATATAAAACAGGGGCAAGGCACAGACTCATAGCAGAGCAATCACCACCAAGCCTGGAATAACTGCAGCCACC |
| 95 | >SP0240 |
| | CAGGCTTTCACTTTCTCGCCAACTTACAAGGCCTTTCTGTGTAAACAATACCTGAACCTTTACCCCGTTGCCCGGCAACGGCCAGGTCTGTGCCAAGTGTTTGCCGATGACCTAATGATTCTGAGCTTGGCAAAGGTCTTATCTC |
| | CCAGCTCGCCCAGGCCCAGTGTTCCAGGAATGTGACCTTTGCTGCAGCAGCCGCTGGAGGGGGCAGAGGGGATGGGCTGGAGGTTGAGCAAACAGAGCAGCAGAAAAGGCAGTTCCTCTTCTCCAGTGCCCTCCTTCCCTGTCTC |
| | TGCCTCTCCCTCCCTTCCTCAGGCATCAGAGCGGAGACTTCAGGGAGACCAGAGCCCAGCTTGCCAGGCACTGAGCTAGAAGCCCTGCC |
| 96 | > SP0246 |
| | AGGTTAATTTTTAAAAAGCAGTCAAAAGTCCAAGTGGCCCTTGGCAGCATTTACTCTCTCTGTTTGCTCTGGTTAATAATCTCAGGAGCACAAACATTCCGGCCCGGGAGGCGCCCTTTGGACCTTTTGCAATCCTGGCGCACTG |
| | AACCCTTGACCCCTGCCCTGCAGCCCCCGCAGCTTGCTGTTTGCCCACTCTATTTGCCCAGCCCCAGGGGCATATAAAACAGGGGCAAGGCACAGACTCATAGCAGAGCAATCACCACCAAGCCTGGAATAACTGCAGCCACC |
| 144 | > SP0131 |
| | GGCCCGGGAGGCGCCCTTTGGACCTTTTGCAATCCTGGCGCACTGAACCCTTGACCCCTGCCCTGCAGCCCCCGCAGCTTGCTGTTTGCCCACTCTATTTGCCCAGCCCCAGCCCTGGAGAGTCCTTTAGCAGGGCAAAGTGCAA |
| | CATAGGCAGACCTTAAGGGATGACTCAGTAACAGATAAGCTTTGTGTGCCTGCAGGGCATATAAAACAGGGGCAAGGCACAGACTCATAGCAGAGCAATCACCACCAAGCCTGGAATAACTGCAGCCACC (SEQ ID NO: |
| | 144), |
| 145 | > CMV-IE 5′ UTR |
| | TCAGATCGCCTGGAGACGCCATCCACGCTGTTTTGACCTCCATAGAAGACACCGGGACCGATCCAGCCTCCGCGGCCGGGAACGGTGCATTGGAACGCGGATTCCCCGTGCCAAGAGTGACGTAAGTACCGCCTATAGACTCTAT |
| | AGGCACACCCCTTTGGCTCTTATGCATGAACGGTGGAGGGCAGTGTAGTCTGAGCAGTACTCGTTGCTGCCGCGCGCGCCACCAGACATAATAGCTGACAGACTAACAGACTGTTCCTTTCCATGGGTCTTTTCTGCAG (SEQ |
| | ID NO: 145), |
| 146 | > SP0265-UTR |
| | AGGTTAATTTTTAAAAAGCAGTCAAAAGTCCAAGTGGCCCTTGGCAGCATTTACTCTCTCTGTTTGCTCTGGTTAATAATCTCAGGAGCACAAACATTCCTGTACCGGCCCGGGAGGCGCCCTTTGGACCTTTTGCAATCCTGGC |
| | GCACTGAACCCTTGACCCCTGCCCTGCAGCCCCCGCAGCTTGCTGTTTGCCCACTCTATTTGCCCAGCCCCAGCCCTGGAGAGTCCTTTAGCAGGGCAAAGTGCAACATAGGCAGACCTTAAGGGATGACTCAGTAACAGATAAG |
| | CTTTGTGTGCCTGCAGGGCATATAAAACAGGGGCAAGGCACAGACTCATAGCAGAGTCAGATCGCCTGGAGACGCCATCCACGCTGTTTTGACCTCCATAGAAGACACCGGGACCGATCCAGCCTCCGCGGCCGGGAACGGTGCA |
| | TTGGAACGCGGATTCCCCGTGCCAAGAGTGACGTAAGTACCGCCTATAGACTCTATAGGCACACCCCTTTGGCTCTTATGCATGAACGGTGGAGGGCAGTGTAGTCTGAGCAGTACTCGTTGCTGCCGCGCGCGCCACCAGACAT |
| | AATAGCTGACAGACTAACAGACTGTTCCTTTCCATGGGTCTTTTCTGCAGGCCACC (SEQ ID NO: 147 |
| 147 | > SP0239-UTR |
| | CAGGCTTTCACTTTCTCGCCAACTTACAAGGCCTTTCTGTGTAAACAATACCTGAACCTTTACCCCGTTGCCCGGCAACGGCCAGGTCTGTGCCAAGTGTTTGAGGTTAATTTTTAAAAAGCAGTCAAAAGTCCAAGTGGCCCTT |
| | GGCAGCATTTACTCTCTCTGTTTGCTCTGGTTAATAATCTCAGGAGCACAAACATTCCGGCCCGGGAGGCGCCCTTTGGACCTTTTGCAATCCTGGCGCACTGAACCCTTGACCCCTGCCCTGCAGCCCCCGCAGCTTGCTGTTT |
| | GCCCACTCTATTTGCCCAGCCCCAGCCCTGGAGAGTCCTTTAGCAGGGCAAAGTGCAACATAGGCAGACCTTAAGGGATGACTCAGTAACAGATAAGCTTTGTGTGCCTGCAGGGCATATAAAACAGGGGCAAGGCACAGACTCA |
| | TAGCAGAGTCAGATCGCCTGGAGACGCCATCCACGCTGTTTTGACCTCCATAGAAGACACCGGGACCGATCCAGCCTCCGCGGCCGGGAACGGTGCATTGGAACGCGGATTCCCCGTGCCAAGAGTGACGTAAGTACCGCCTATA |
| | GACTCTATAGGCACACCCCTTTGGCTCTTATGCATGAACGGTGGAGGGCAGTGTAGTCTGAGCAGTACTCGTTGCTGCCGCGCGCGCCACCAGACATAATAGCTGACAGACTAACAGACTGTTCCTTTCCATGGGTCTTTTCTGC |
| | AGGCCACC (SEQ ID NO: 147), |
| 148 | > SP0240-UTR |
| | CAGGCTTTCACTTTCTCGCCAACTTACAAGGCCTTTCTGTGTAAACAATACCTGAACCTTTACCCCGTTGCCCGGCAACGGCCAGGTCTGTGCCAAGTGTTTGCCGATGACCTAATGATTCTGAGCTTGGCAAAGGTCTTATCTC |
| | CCAGCTCGCCCAGGCCCAGTGTTCCAGGAATGTGACCTTTGCTGCAGCAGCCGCTGGAGGGGGCAGAGGGGATGGGCTGGAGGTTGAGCAAACAGAGCAGCAGAAAAGGCAGTTCCTCTTCTCCAGTGCCCTCCTTCCCTGTCTC |
| | TGCCTCTCCCTCCCTTCCTCAGGCATCAGAGCGGAGACTTCAGGGAGACCAGAGCCCAGCTTGCCAGGCACTGTCAGATCGCCTGGAGACGCCATCCACGCTGTTTTGACCTCCATAGAAGACACCGGGACCGATCCAGCCTCCG |
| | CGGCCGGGAACGGTGCATTGGAACGCGGATTCCCCGTGCCAAGAGTGACGTAAGTACCGCCTATAGACTCTATAGGCACACCCCTTTGGCTCTTATGCATGAACGGTGGAGGGCAGTGTAGTCTGAGCAGTACTCGTTGCTGCCG |
| | CGCGCGCCACCAGACATAATAGCTGACAGACTAACAGACTGTTCCTTTCCATGGGTCTTTTCTGCAGGCCACC (SEQ ID NO: 148 |
| 149 | > SP0246-UTR |
| | AGGTTAATTTTTAAAAAGCAGTCAAAAGTCCAAGTGGCCCTTGGCAGCATTTACTCTCTCTGTTTGCTCTGGTTAATAATCTCAGGAGCACAAACATTCCGGCCCGGGAGGCGCCCTTTGGACCTTTTGCAATCCTGGCGCACTG |
| | AACCCTTGACCCCTGCCCTGCAGCCCCCGCAGCTTGCTGTTTGCCCACTCTATTTGCCCAGCCCCAGGGGCATATAAAACAGGGGCAAGGCACAGACTCATAGCAGAGTCAGATCGCCTGGAGACGCCATCCACGCTGTTTTGAC |
| | CTCCATAGAAGACACCGGGACCGATCCAGCCTCCGCGGCCGGGAACGGTGCATTGGAACGCGGATTCCCCGTGCCAAGAGTGACGTAAGTACCGCCTATAGACTCTATAGGCACACCCCTTTGGCTCTTATGCATGAACGGTGGA |
| | GGGCAGTGTAGTCTGAGCAGTACTCGTTGCTGCCGCGCGCGCCACCAGACATAATAGCTGACAGACTAACAGACTGTTCCTTTCCATGGGTCTTTTCTGCAGGCCACC (SEQ ID NO: 149), |
| 150 | > SP0131_A1-UTR |
| | GGCCCGGGAGGCGCCCTTTGGACCTTTTGCAATCCTGGCGCACTGAACCCTTGACCCCTGCCCTGCAGCCCCCGCAGCTTGCTGTTTGCCCACTCTATTTGCCCAGCCCCAGCCCTGGAGAGTCCTTTAGCAGGGCAAAGTGCAA |
| | CATAGGCAGACCTTAAGGGATGACTCAGTAACAGATAAGCTTTGTGTGCCTGCAGGGCATATAAAACAGGGGCAAGGCACAGACTCATAGCAGAGTCAGATCGCCTGGAGACGCCATCCACGCTGTTTTGACCTCCATAGAAGAC |
| | ACCGGGACCGATCCAGCCTCCGCGGCCGGGAACGGTGCATTGGAACGCGGATTCCCCGTGCCAAGAGTGACGTAAGTACCGCCTATAGACTCTATAGGCACACCCCTTTGGCTCTTATGCATGAACGGTGGAGGGCAGTGTAGTC |
| | TGAGCAGTACTCGTTGCTGCCGCGCGCGCCACCAGACATAATAGCTGACAGACTAACAGACTGTTCCTTTCCATGGGTCTTTTCTGCAGGCCACC (SEQ ID NO: 150), |
| 154 | >LVR412_askbioEU |
| | GCCATTCGCCATTCAGGCTGCGCAACTGTTGGGAAGGGCGATCGGTGCGGGCCTCTTCGCTATTACGCCAGCTGGCGAAAGGGGGATGTGCTGCAAGGCGATTAAGTTGGGTAACGCCAGGGTTTTCCCAGTCACGACGTTGTAA |
| | AACGACGGCCAGTGAATTCCTCGAGATTTAAATGACGTTGGCCACTCCCTCTCTGCGCGCTCGCTCGCTCACTGAGGCCGGGCGACCAAAGGTCGCCCGACGCCCGGGCTTTGCCCGGGCGGCCTCAGTGAGCGAGCGAGCGCGC |
| | AGAGAGGGAGTGGCCAACTCCATCACTAGGGGTTCCTGAGTTTAAACTTCGTCGAGGCCGCACGCGTAGGTTAATTTTTAAAAAGCAGTCAAAAGTCCAAGTGGCCCTTGGCAGCATTTACTCTCTCTGTTTGCTCTGGTTAATA |
| | ATCTCAGGAGCACAAACATTCCCCCTGTTCAAACATGTCCTAATACTCTGTCTCTGCAAGGGTCATCAGTAGTTTTCCATCTTACTCAACATCCTCCCAGTGAGTCATATGTTTGCTCACTGAAGGTTACTAGTTAACAGGCATC |
| | CCTTAAACAGGATATAAAAGGACTTCAGCAGGACTGCTCGAAACATCCCACTCAGCCACCATGGGAGTGAGGCACCCGCCCTGCTCCCACCGGCTCCTGGCCGTCTGCGCCCTCGTGTCCTTGGCAACCGCTGCACTCCTGGGGC |
| | ACATCCTACTCCATGATTTCCTGCTGGTTCCCCGAGAGCTGAGTGGCTCCTCCCCAGTCCTGGAGGAGACTCACCCAGCTCACCAGCAGGGAGCCAGCAGACCAGGGCCCCGGGATGCCCAGGCACACCCCGGCCGTCCCAGAGC |
| | AGTGCCCACACAGTGCGACGTCCCCCCCAACAGCCGCTTCGATTGCGCCCCTGACAAGGCCATCACCCAGGAACAGTGCGAGGCCCGCGGCTGCTGCTACATCCCTGCAAAGCAGGGGCTGCAGGGAGCCCAGATGGGGCAGCCC |
| | TGGTGCTTCTTCCCACCCAGCTACCCCAGCTACAAGCTGGAGAACCTGAGCTCCTCTGAAATGGGCTACACGGCCACCCTGACCCGTACCACCCCCACCTTCTTCCCCAAGGACATCCTGACCCTGCGGCTGGACGTGATGATGG |
| | AGACTGAGAACCGCCTCCACTTCACGATCAAAGATCCAGCTAACAGGCGCTACGAGGTGCCCTTGGAGACCCCGCGTGTCCACAGCCGGGCACCGTCCCCACTCTACAGCGTGGAGTTCTCCGAGGAGCCCTTCGGGGTGATCGT |
| | GCACCGGCAGCTGGACGGCCGCGTGCTGCTGAACACGACGGTGGCGCCCCTGTTCTTTGCGGACCAGTTCCTTCAGCTGTCCACCTCGCTGCCCTCGCAGTATATCACAGGCCTCGCCGAGCACCTCAGTCCCCTGATGCTCAGC |
| | ACCAGCTGGACCAGGATCACCCTGTGGAACCGGGACCTTGCGCCCACGCCCGGTGCGAACCTCTACGGGTCTCACCCTTTCTACCTGGCGCTGGAGGACGGCGGGTCGGCACACGGGGTGTTCCTGCTAAACAGCAATGCCATGG |
| | ATGTGGTCCTGCAGCCGAGCCCTGCCCTTAGCTGGAGGTCGACAGGTGGGATCCTGGATGTCTACATCTTCCTGGGCCCAGAGCCCAAGAGCGTGGTGCAGCAGTACCTGGACGTTGTGGGATACCCGTTCATGCCGCCATACTG |
| | GGGCCTGGGCTTCCACCTGTGCCGCTGGGGCTACTCCTCCACCGCTATCACCCGCCAGGTGGTGGAGAACATGACCAGGGCCCACTTCCCCCTGGACGTCCAATGGAACGACCTGGACTACATGGACTCCCGGAGGGACTTCACG |
| | TTCAACAAGGATGGCTTCCGGGACTTCCCGGCCATGGTGCAGGAGCTGCACCAGGGCGGCCGGCGCTACATGATGATCGTGGATCCTGCCATCAGCAGCTCGGGCCCTGCCGGGAGCTACAGGCCCTACGACGAGGGTCTGCGGA |
| | GGGGGGTTTTCATCACCAACGAGACCGGCCAGCCGCTGATTGGGAAGGTATGGCCCGGGTCCACTGCCTTCCCCGACTTCACCAACCCCACAGCCCTGGCCTGGTGGGAGGACATGGTGGCTGAGTTCCATGACCAGGTGCCCTT |
| | CGACGGCATGTGGATTGACATGAACGAGCCTTCCAACTTCATCAGAGGCTCTGAGGACGGCTGCCCCAACAATGAGCTGGAGAACCCACCCTACGTGCCTGGGGTGGTTGGGGGGACCCTCCAGGCGGCCACCATCTGTGCCTCC |
| | AGCCACCAGTTTCTCTCCACACACTACAACCTGCACAACCTCTACGGCCTGACCGAAGCCATCGCCTCCCACAGGGCGCTGGTGAAGGCTCGGGGGACACGCCCATTTGTGATCTCCCGCTCGACCTTTGCTGGCCACGGCCGAT |
| | ACGCCGGCCACTGGACGGGGGACGTGTGGAGCTCCTGGGAGCAGCTCGCCTCCTCCGTGCCAGAAATCCTGCAGTTTAACCTGCTGGGGGTGCCTCTGGTCGGGGCCGACGTCTGCGGCTTCCTGGGCAACACCTCAGAGGAGCT |
| | GTGTGTGCGCTGGACCCAGCTGGGGGCCTTCTACCCCTTCATGCGGAACCACAACAGCCTGCTCAGTCTGCCCCAGGAGCCGTACAGCTTCAGCGAGCCGGCCCAGCAGGCCATGAGGAAGGCCCTCACCCTGCGCTACGCACTC |
| | CTCCCCCACCTCTACACACTGTTCCACCAGGCCCACGTCGCGGGGGAGACCGTGGCCCGGCCCCTCTTCCTGGAGTTCCCCAAGGACTCTAGCACCTGGACTGTGGACCACCAGCTCCTGTGGGGGGAGGCCCTGCTCATCACCC |
| | CAGTGCTCCAGGCCGGGAAGGCCGAAGTGACTGGCTACTTCCCCTTGGGCACATGGTACGACCTGCAGACGGTGCCAATAGAGGCCCTTGGCAGCCTCCCACCCCCACCTGCAGCTCCCCGTGAGCCAGCCATCCACAGCGAGGG |
| | GCAGTGGGTGACGCTGCCGGCCCCCCTGGACACCATCAACGTCCACCTCCGGGCTGGGTACATCATCCCCCTGCAGGGCCCTGGCCTCACAACCACAGAGTCCCGCCAGCAGCCCATGGCCCTGGCTGTGGCCCTGACCAAGGGT |
| | GGAGAGGCCCGAGGGGAGCTGTTCTGGGACGATGGAGAGAGCCTGGAAGTGCTGGAGCGAGGGGCCTACACACAGGTCATCTTCCTGGCCAGGAATAACACGATCGTGAATGAGCTGGTACGTGTGACCAGTGAGGGAGCTGGCC |
| | TGCAGCTGCAGAAGGTGACTGTCCTGGGCGTGGCCACGGCGCCCCAGCAGGTCCTCTCCAACGGTGTCCCTGTCTCCAACTTCACCTACAGCCCCGACACCAAGGTCCTGGACATCTGTGTCTCGCTGTTGATGGGAGAGCAGTT |
| | TCTCGTCAGCTGGTGTTAGCCGGGCGGAGTGTGTTAGTCTCTCCAGAGGGAGGCTGGTTCCCCAGGGAAGCAGAGCCTGTGTGCGGGCAGCAGCTGTGTGCGGGCCTGGGGGTTGTTAAGTGCAATTATTTTTAATAAAAGGGGC |
| | ATTTGGAAAAAAAAAAAAAAGGTAGCAGTCGACAGATGAATTCTGCAGATCTGTGGCTTCTAGCTGCCCGGGTGGCATCCCTGTGACCCCTCCCCAGTGCCTCTCCTGGCCCTGGAAGTTGCCACTCCAGTGCCCACCAGCCTTG |
| | TCCTAATAAAATTAAGTTGCATCATTTTGTCTGACTAGGTGTCCTTCTATAATATTATGGGGTGGAGGGGGGTGGTATGGAGCAAGGGGCAAGTTGGGAAGACAACCTGAGTTGTTGGGATTCCAGGCATCGAGTAGATAAGTAG |
| | CATGGCGGGTTAATCATTAACTACACACGTGCGGACCGAGCGGCCTAAGCTCGCGAAGGAACCCCTAGTGATGGAGTTGGCCACTCCCTCTCTGCGCGCTCGCTCGCTCACTGAGGCCGGGCGACCAAAGGTCGCCCGACGCCCG |
| | GGCTTTGCCCGGGCGGCCTCAGTGAGCGAGCGAGCGCGCAGAGAGGGAGTGGCCAAGACGATTTAAATGACAAGCTTGGCGTAATCATGGTCATAGCTGTTTCCTGTGTGAAATTGTTATCCGCTCACAATTCCACACAACATAC |
| | GAGCCGGAAGCATAAAGTGTAAAGCCTGGGGTGCCTAATGAGTGAGCTAACTCACATTAATTGCGTTGCGCTCACTGCCCGCTTTCCAGTCGGGAAACCTGTCGTGCCAGCTGCATTAATGAATCGGCCAACGCGCGGGGAGAGG |
| | CGGTTTGCGTATTGGGCGCTCTTCCGCTTCCTCGCTCACTGACTCGCTGCGCTCGGTCGTTCGGCTGCGGCGAGCGGTATCAGCTCACTCAAAGGCGGTAATACGGTTATCCACAGAATCAGGGGATAACGCAGGAAAGAACATG |
| | TGAGCAAAAGGCCAGCAAAAGGCCAGGAACCGTAAAAAGGCCGCGTTGCTGGCGTTTTTCCATAGGCTCCGCCCCCCTGACGAGCATCACAAAAATCGACGCTCAAGTCAGAGGTGGCGAAACCCGACAGGACTATAAAGATACC |
| | AGGCGTTTCCCCCTGGAAGCTCCCTCGTGCGCTCTCCTGTTCCGACCCTGCCGCTTACCGGATACCTGTCCGCCTTTCTCCCTTCGGGAAGCGTGGCGCTTTCTCATAGCTCACGCTGTAGGTATCTCAGTTCGGTGTAGGTCGT |
| | TCGCTCCAAGCTGGGCTGTGTGCACGAACCCCCCGTTCAGCCCGACCGCTGCGCCTTATCCGGTAACTATCGTCTTGAGTCCAACCCGGTAAGACACGACTTATCGCCACTGGCAGCAGCCACTGGTAACAGGATTAGCAGAGCG |
| | AGGTATGTAGGCGGTGCTACAGAGTTCTTGAAGTGGTGGCCTAACTACGGCTACACTAGAAGAACAGTATTTGGTATCTGCGCTCTGCTGAAGCCAGTTACCTTCGGAAAAAGAGTTGGTAGCTCTTGATCCGGCAAACAAACCA |
| | CCGCTGGTAGCGGTGGTTTTTTTGTTTGCAAGCAGCAGATTACGCGCAGAAAAAAAGGATCTCAAGAAGATCCTTTGATCTTTTCTACGGGGTCTGACGCTCAGTGGAACGAAAACTCACGTTAAGGGATTTTGGTCATGAGATT |
| | ATCAAAAAGGATCTTCACCTAGATCCTTTTAAATTAAAAATGAAGTTTTAAATCAATCTAAAGTATATATGAGTAAACTTGGTCTGACAGTTAGAAAAACTCATCGAGCATCAAATGAAACTGCAATTTATTCATATCAGGATTA |
| | TCAATACCATATTTTTGAAAAAGCCGTTTCTGTAATGAAGGAGAAAACTCACCGAGGCAGTTCCATAGGATGGCAAGATCCTGGTATCGGTCTGCGATTCCGACTCGTCCAACATCAATACAACCTATTAATTTCCCCTCGTCAA |
| | AAATAAGGTTATCAAGTGAGAAATCACCATGAGTGACGACTGAATCCGGTGAGAATGGCAAAAGTTTATGCATTTCTTTCCAGACTTGTTCAACAGGCCAGCCATTACGCTCGTCATCAAAATCACTCGCATCAACCAAACCGTT |
| | ATTCATTCGTGATTGCGCCTGAGCGAGACGAAATACGCGATCGCTGTTAAAAGGACAATTACAAACAGGAATCGAATGCAACCGGCGCAGGAACACTGCCAGCGCATCAACAATATTTTCACCTGAATCAGGATATTCTTCTAAT |
| | ACCTGGAATGCTGTTTTTCCGGGGATCGCAGTGGTGAGTAACCATGCATCATCAGGAGTACGGATAAAATGCTTGATGGTCGGAAGAGGCATAAATTCCGTCAGCCAGTTTAGTCTGACCATCTCATCTGTAACATCATTGGCAA |
| | CGCTACCTTTGCCATGTTTCAGAAACAACTCTGGCGCATCGGGCTTCCCATACAAGCGATAGATTGTCGCACCTGATTGCCCGACATTATCGCGAGCCCATTTATACCCATATAAATCAGCATCCATGTTGGAATTTAATCGCGG |
| | CCTCGACGTTTCCCGTTGAATATGGCTCATACTCTTCCTTTTTCAATATTATTGAAGCATTTATCAGGGTTATTGTCTCATGAGCGGATACATATTTGAATGTATTTAGAAAAATAAACAAATAGGGGTTCCGCGCACATTTCCC |
| | CGAAAAGTGCCACCTGACGTCTAAGAAACCATTATTATCATGACATTAACCTATAAAAATAGGCGTATCACGAGGCCCTTTCGTCTCGCGCGTTTCGGTGATGACGGTGAAAACCTCTGACACATGCAGCTCCCGGAGACGGTCA |
| | CAGCTTGTCTGTAAGCGGATGCCGGGAGCAGACAAGCCCGTCAGGGCGCGTCAGCGGGTGTTGGCGGGTGTCGGGGCTGGCTTAACTATGCGGCATCAGAGCAGATTGTACTGAGAGTGCACCATATGCGGTGTGAAATACCGCA |
| | CAGATGCGTAAGGAGAAAATACCGCATCAGGC |
| 158 | >LVR422 Stuffer |
| | GCCATTCGCCATTCAGGCTGCGCAACTGTTGGGAAGGGCGATCGGTGCGGGCCTCTTCGCTATTACGCCAGCTGGCGAAAGGGGGATGTGCTGCAAGGCGATTAAGTTGGGTAACGCCAGGGTTTTCCCAGTCACGACGTTGTAA |
| | AACGACGGCCAGTGAATTCCTCGAGATTTAAATGACGTTGGCCACTCCCTCTCTGCGCGCTCGCTCGCTCACTGAGGCCGGGCGACCAAAGGTCGCCCGACGCCCGGGCTTTGCCCGGGCGGCCTCAGTGAGCGAGCGAGCGCGC |
| | AGAGAGGGAGTGGCCAACTCCATCACTAGGGGTTCCTGAGTTTAAACTTCGTCGAGGCCGCACGCGTAGGTTAATTTTTAAAAAGCAGTCAAAAGTCCAAGTGGCCCTTGGCAGCATTTACTCTCTCTGTTTGCTCTGGTTAATA |
| | ATCTCAGGAGCACAAACATTCCCCCTGTTCAAACATGTCCTAATACTCTGTCTCTGCAAGGGTCATCAGTAGTTTTCCATCTTACTCAACATCCTCCCAGTGAGTCATATGTTTGCTCACTGAAGGTTACTAGTTAACAGGCATC |
| | CCTTAAACAGGATATAAAAGGACTTCAGCAGGACTGCTCGAAACATCCCACTTCAGATCGCCTGGAGACGCCATCCACGCTGTTTTGACCTCCATAGAAGACACCGGGACCGATCCAGCCTCCGCGGCCGGGAACGGTGCATTGG |
| | AACGCGGATTCCCCGTGCCAAGAGTGACGTAAGTACCGCCTATAGACTCTATAGGCACACCCCTTTGGCTCTTATGCATGAACGGTGGAGGGCAGTGTAGTCTGAGCAGTACTCGTTGCTGCCGCGCGCGCCACCAGACATAATA |
| | GCTGACAGACTAACAGACTGTTCCTTTCCATGGGTCTTTTCTGCAGGCCACCATGGGAGTGAGGCACCCGCCCTGCTCCCACCGGCTCCTGGCCGTCTGCGCCCTCGTGTCCTTGGCAACCGCTGCACTCCTGGGGCACATCCTA |
| | CTCCATGATTTCCTGCTGGTTCCCCGAGAGCTGAGTGGCTCCTCCCCAGTCCTGGAGGAGACTCACCCAGCTCACCAGCAGGGAGCCAGCAGACCAGGGCCCCGGGATGCCCAGGCACACCCCGGCCGTCCCAGAGCAGTGCCCA |
| | CACAGTGCGACGTCCCCCCCAACAGCCGCTTCGATTGCGCCCCTGACAAGGCCATCACCCAGGAACAGTGCGAGGCCCGCGGCTGCTGCTACATCCCTGCAAAGCAGGGGCTGCAGGGAGCCCAGATGGGGCAGCCCTGGTGCTT |
| | CTTCCCACCCAGCTACCCCAGCTACAAGCTGGAGAACCTGAGCTCCTCTGAAATGGGCTACACGGCCACCCTGACCCGTACCACCCCCACCTTCTTCCCCAAGGACATCCTGACCCTGCGGCTGGACGTGATGATGGAGACTGAG |
| | AACCGCCTCCACTTCACGATCAAAGATCCAGCTAACAGGCGCTACGAGGTGCCCTTGGAGACCCCGCGTGTCCACAGCCGGGCACCGTCCCCACTCTACAGCGTGGAGTTCTCCGAGGAGCCCTTCGGGGTGATCGTGCACCGGC |
| | AGCTGGACGGCCGCGTGCTGCTGAACACGACGGTGGCGCCCCTGTTCTTTGCGGACCAGTTCCTTCAGCTGTCCACCTCGCTGCCCTCGCAGTATATCACAGGCCTCGCCGAGCACCTCAGTCCCCTGATGCTCAGCACCAGCTG |
| | GACCAGGATCACCCTGTGGAACCGGGACCTTGCGCCCACGCCCGGTGCGAACCTCTACGGGTCTCACCCTTTCTACCTGGCGCTGGAGGACGGCGGGTCGGCACACGGGGTGTTCCTGCTAAACAGCAATGCCATGGATGTGGTC |
| | CTGCAGCCGAGCCCTGCCCTTAGCTGGAGGTCGACAGGTGGGATCCTGGATGTCTACATCTTCCTGGGCCCAGAGCCCAAGAGCGTGGTGCAGCAGTACCTGGACGTTGTGGGATACCCGTTCATGCCGCCATACTGGGGCCTGG |
| | GCTTCCACCTGTGCCGCTGGGGCTACTCCTCCACCGCTATCACCCGCCAGGTGGTGGAGAACATGACCAGGGCCCACTTCCCCCTGGACGTCCAATGGAACGACCTGGACTACATGGACTCCCGGAGGGACTTCACGTTCAACAA |
| | GGATGGCTTCCGGGACTTCCCGGCCATGGTGCAGGAGCTGCACCAGGGCGGCCGGCGCTACATGATGATCGTGGATCCTGCCATCAGCAGCTCGGGCCCTGCCGGGAGCTACAGGCCCTACGACGAGGGTCTGCGGAGGGGGGTT |
| | TTCATCACCAACGAGACCGGCCAGCCGCTGATTGGGAAGGTATGGCCCGGGTCCACTGCCTTCCCCGACTTCACCAACCCCACAGCCCTGGCCTGGTGGGAGGACATGGTGGCTGAGTTCCATGACCAGGTGCCCTTCGACGGCA |
| | TGTGGATTGACATGAACGAGCCTTCCAACTTCATCAGAGGCTCTGAGGACGGCTGCCCCAACAATGAGCTGGAGAACCCACCCTACGTGCCTGGGGTGGTTGGGGGGACCCTCCAGGCGGCCACCATCTGTGCCTCCAGCCACCA |
| | GTTTCTCTCCACACACTACAACCTGCACAACCTCTACGGCCTGACCGAAGCCATCGCCTCCCACAGGGCGCTGGTGAAGGCTCGGGGGACACGCCCATTTGTGATCTCCCGCTCGACCTTTGCTGGCCACGGCCGATACGCCGGC |
| | CACTGGACGGGGGACGTGTGGAGCTCCTGGGAGCAGCTCGCCTCCTCCGTGCCAGAAATCCTGCAGTTTAACCTGCTGGGGGTGCCTCTGGTCGGGGCCGACGTCTGCGGCTTCCTGGGCAACACCTCAGAGGAGCTGTGTGTGC |
| | GCTGGACCCAGCTGGGGGCCTTCTACCCCTTCATGCGGAACCACAACAGCCTGCTCAGTCTGCCCCAGGAGCCGTACAGCTTCAGCGAGCCGGCCCAGCAGGCCATGAGGAAGGCCCTCACCCTGCGCTACGCACTCCTCCCCCA |
| | CCTCTACACACTGTTCCACCAGGCCCACGTCGCGGGGGAGACCGTGGCCCGGCCCCTCTTCCTGGAGTTCCCCAAGGACTCTAGCACCTGGACTGTGGACCACCAGCTCCTGTGGGGGGAGGCCCTGCTCATCACCCCAGTGCTC |
| | CAGGCCGGGAAGGCCGAAGTGACTGGCTACTTCCCCTTGGGCACATGGTACGACCTGCAGACGGTGCCAATAGAGGCCCTTGGCAGCCTCCCACCCCCACCTGCAGCTCCCCGTGAGCCAGCCATCCACAGCGAGGGGCAGTGGG |
| | TGACGCTGCCGGCCCCCCTGGACACCATCAACGTCCACCTCCGGGCTGGGTACATCATCCCCCTGCAGGGCCCTGGCCTCACAACCACAGAGTCCCGCCAGCAGCCCATGGCCCTGGCTGTGGCCCTGACCAAGGGTGGAGAGGC |
| | CCGAGGGGAGCTGTTCTGGGACGATGGAGAGAGCCTGGAAGTGCTGGAGCGAGGGGCCTACACACAGGTCATCTTCCTGGCCAGGAATAACACGATCGTGAATGAGCTGGTACGTGTGACCAGTGAGGGAGCTGGCCTGCAGCTG |
| | CAGAAGGTGACTGTCCTGGGCGTGGCCACGGCGCCCCAGCAGGTCCTCTCCAACGGTGTCCCTGTCTCCAACTTCACCTACAGCCCCGACACCAAGGTCCTGGACATCTGTGTCTCGCTGTTGATGGGAGAGCAGTTTCTCGTCA |
| | GCTGGTGTTAGCGAGCGGCCGCTCTTAGTAGCAGTATCGATCCCAGCCCACTTTTCCCCAATACGACTACGAGATCTGTGGCTTCTAGCTGCCCGGGTGGCATCCCTGTGACCCCTCCCCAGTGCCTCTCCTGGCCCTGGAAGTT |
| | GCCACTCCAGTGCCCACCAGCCTTGTCCTAATAAAATTAAGTTGCATCATTTTGTCTGACTAGGTGTCCTTCTATAATATTATGGGGTGGAGGGGGGTGGTATGGAGCAAGGGGCAAGTTGGGGTCCTTCAACGTCGAACTGTAG |
| | CACTCTGGATAGCTAGCTAGCGTTTGTAGATAGTTTCAAGGATAGCAACCAACAACACGATAGTTTAGAAATTGTTCTAGATCTTCTGCTACTACTCGAGAGATAGTTGACTTCTAGAAGACTCTGGATAGAGAGCTTCCTGTGG |
| | TTTCTATTTCAACACTTCGAGCGATTTAGATAGAGGCGTCAATTGAGAACAAAAACTGGACACCTGTTTCTAGAACACTAGTGCTAGTCTTCTCAAGGCTTGATACGTCCTGCCACTCACACACAAAAAACCAACACACAGATTA |
| | ATGAAAATAAAGATCTTTTATTAGAGAGCGAGTGGATAGTTTAGAGTCGCTTGAGACTTATCCTGTGTCCAACTAGCCAAGAGTAGAGAGAGAGTGACTAGGTGGATCGAGAGATCGTCGTCTATTTGACTGCTAGCCTGTGTTC |
| | TCGTCGTAGTCCAAATCTTCAATACTCTGGCGACTAGTAACTTGAATCTGTGTATCTAACACGACAAGGCTACTCCTTTGAGTTTCTAGTGATAAGTGCGACTGGATCTAGCTAGTTGAGTTTCTAGAAACTACGTCCAAAGCTA |
| | CTAGCACTACACGTGCGGACCGAGCGGCCTAAGCTCGCGAAGGAACCCCTAGTGATGGAGTTGGCCACTCCCTCTCTGCGCGCTCGCTCGCTCACTGAGGCCGGGCGACCAAAGGTCGCCCGACGCCCGGGCTTTGCCCGGGCGG |
| | CCTCAGTGAGCGAGCGAGCGCGCAGAGAGGGAGTGGCCAAGACGATTTAAATGACAAGCTTGGCGTAATCATGGTCATAGCTGTTTCCTGTGTGAAATTGTTATCCGCTCACAATTCCACACAACATACGAGCCGGAAGCATAAA |
| | GTGTAAAGCCTGGGGTGCCTAATGAGTGAGCTAACTCACATTAATTGCGTTGCGCTCACTGCCCGCTTTCCAGTCGGGAAACCTGTCGTGCCAGCTGCATTAATGAATCGGCCAACGCGCGGGGAGAGGCGGTTTGCGTATTGGG |
| | CGCTCTTCCGCTTCCTCGCTCACTGACTCGCTGCGCTCGGTCGTTCGGCTGCGGCGAGCGGTATCAGCTCACTCAAAGGCGGTAATACGGTTATCCACAGAATCAGGGGATAACGCAGGAAAGAACATGTGAGCAAAAGGCCAGC |
| | AAAAGGCCAGGAACCGTAAAAAGGCCGCGTTGCTGGCGTTTTTCCATAGGCTCCGCCCCCCTGACGAGCATCACAAAAATCGACGCTCAAGTCAGAGGTGGCGAAACCCGACAGGACTATAAAGATACCAGGCGTTTCCCCCTGG |
| | AAGCTCCCTCGTGCGCTCTCCTGTTCCGACCCTGCCGCTTACCGGATACCTGTCCGCCTTTCTCCCTTCGGGAAGCGTGGCGCTTTCTCATAGCTCACGCTGTAGGTATCTCAGTTCGGTGTAGGTCGTTCGCTCCAAGCTGGGC |
| | TGTGTGCACGAACCCCCCGTTCAGCCCGACCGCTGCGCCTTATCCGGTAACTATCGTCTTGAGTCCAACCCGGTAAGACACGACTTATCGCCACTGGCAGCAGCCACTGGTAACAGGATTAGCAGAGCGAGGTATGTAGGCGGTG |
| | CTACAGAGTTCTTGAAGTGGTGGCCTAACTACGGCTACACTAGAAGAACAGTATTTGGTATCTGCGCTCTGCTGAAGCCAGTTACCTTCGGAAAAAGAGTTGGTAGCTCTTGATCCGGCAAACAAACCACCGCTGGTAGCGGTGG |
| | TTTTTTTGTTTGCAAGCAGCAGATTACGCGCAGAAAAAAAGGATCTCAAGAAGATCCTTTGATCTTTTCTACGGGGTCTGACGCTCAGTGGAACGAAAACTCACGTTAAGGGATTTTGGTCATGAGATTATCAAAAAGGATCTTC |
| | ACCTAGATCCTTTTAAATTAAAAATGAAGTTTTAAATCAATCTAAAGTATATATGAGTAAACTTGGTCTGACAGTTAGAAAAACTCATCGAGCATCAAATGAAACTGCAATTTATTCATATCAGGATTATCAATACCATATTTTT |
| | GAAAAAGCCGTTTCTGTAATGAAGGAGAAAACTCACCGAGGCAGTTCCATAGGATGGCAAGATCCTGGTATCGGTCTGCGATTCCGACTCGTCCAACATCAATACAACCTATTAATTTCCCCTCGTCAAAAATAAGGTTATCAAG |
| | TGAGAAATCACCATGAGTGACGACTGAATCCGGTGAGAATGGCAAAAGTTTATGCATTTCTTTCCAGACTTGTTCAACAGGCCAGCCATTACGCTCGTCATCAAAATCACTCGCATCAACCAAACCGTTATTCATTCGTGATTGC |
| | GCCTGAGCGAGACGAAATACGCGATCGCTGTTAAAAGGACAATTACAAACAGGAATCGAATGCAACCGGCGCAGGAACACTGCCAGCGCATCAACAATATTTTCACCTGAATCAGGATATTCTTCTAATACCTGGAATGCTGTTT |
| | TTCCGGGGATCGCAGTGGTGAGTAACCATGCATCATCAGGAGTACGGATAAAATGCTTGATGGTCGGAAGAGGCATAAATTCCGTCAGCCAGTTTAGTCTGACCATCTCATCTGTAACATCATTGGCAACGCTACCTTTGCCATG |
| | TTTCAGAAACAACTCTGGCGCATCGGGCTTCCCATACAAGCGATAGATTGTCGCACCTGATTGCCCGACATTATCGCGAGCCCATTTATACCCATATAAATCAGCATCCATGTTGGAATTTAATCGCGGCCTCGACGTTTCCCGT |
| | TGAATATGGCTCATACTCTTCCTTTTTCAATATTATTGAAGCATTTATCAGGGTTATTGTCTCATGAGCGGATACATATTTGAATGTATTTAGAAAAATAAACAAATAGGGGTTCCGCGCACATTTCCCCGAAAAGTGCCACCTG |
| | ACGTCTAAGAAACCATTATTATCATGACATTAACCTATAAAAATAGGCGTATCACGAGGCCCTTTCGTCTCGCGCGTTTCGGTGATGACGGTGAAAACCTCTGACACATGCAGCTCCCGGAGACGGTCACAGCTTGTCTGTAAGC |
| | GGATGCCGGGAGCAGACAAGCCCGTCAGGGCGCGTCAGCGGGTGTTGGCGGGTGTCGGGGCTGGCTTAACTATGCGGCATCAGAGCAGATTGTACTGAGAGTGCACCATATGCGGTGTGAAATACCGCACAGATGCGTAAGGAGA |
| | AAATACCGCATCAGGC |
| | >LVRSP0412 insertion |
| | GGTACCGAGCTCGGATCCACTAGTAACGGCCGCCAGTGTGCTGGAAT |
| 106 | >mod GAA (H199R, R223H) |
| | MGVRHPPCSHRLLAVCALVSLATAALLGHILLHDFLLVPRELSGSSPVLEETHPAHQQGASRPGPRDAQAHPGRPRAVPTQCDVPPNSRFDCAPDKAITQEQCEARGCCYIPAKQGLQGAQMGQPWCFFPPSYPSYKLENLSSSE |
| | MGYTATLTRTTPTFFPKDILTLRLDVMMETENRLHFTIKDPANRRYEVPLETPRVHSRAPSPLYSVEFSEEPFGVIVHRQLDGRVLLNTTVAPLFFADQFLQLSTSLPSQYITGLAEHLSPLMLSTSWTRITLWNRDLAPTPGAN |
| | LYGSHPFYLALEDGGSAHGVFLLNSNAMDVVLQPSPALSWRSTGGILDVYIFLGPEPKSVVQQYLDVVGYPFMPPYWGLGFHLCRWGYSSTAITRQVVENMTRAHFPLDVQWNDLDYMDSRRDFTFNKDGFRDFPAMVQELHQGG |
| | RRYMMIVDPAISSSGPAGSYRPYDEGLRRGVFITNETGQPLIGKVWPGSTAFPDFTNPTALAWWEDMVAEFHDQVPFDGMWIDMNEPSNFIRGSEDGCPNNELENPPYVPGVVGGTLQAATICASSHQFLSTHYNLHNLYGLTEA |
| | IASHRALVKARGTRPFVISRSTFAGHGRYAGHWTGDVWSSWEQLASSVPEILQFNLLGVPLVGADVCGFLGNTSEELCVRWTQLGAFYPFMRNHNSLLSLPQEPYSFSEPAQQAMRKALTLRYALLPHLYTLFHQAHVAGETVAR |
| | PLFLEFPKDSSTWTVDHQLLWGEALLITPVLQAGKAEVTGYFPLGTWYDLQTVPIEALGSLPPPPAAPREPAIHSEGQWVTLPAPLDTINVHLRAGYIIPLQGPGLTTTESRQQPMALAVALTKGGEARGELEWDDGESLEVLER |
| | GAYTQVIFLARNNTIVNELVRVTSEGAGLQLQKVTVLGVATAPQQVLSNGVPVSNFTYSPDTKVLDICVSLLMGEQFLVSWC |
| 107 | >mod GAA (H199R, H203L, R223H) |
| | MGVRHPPCSHRLLAVCALVSLATAALLGHILLHDFLLVPRELSGSSPVLEETHPAHQQGASRPGPRDAQAHPGRPRAVPTQCDVPPNSRFDCAPDKAITQEQCEARGCCYIPAKQGLQGAQMGQPWCFFPPSYPSYKLENLSSSE |
| | MGYTATLTRTTPTFFPKDILTLRLDVMMETENRLHFTIKDPANRRYEVPLETPRVLSRAPSPLYSVEFSEEPFGVIVHRQLDGRVLLNTTVAPLFFADQFLQLSTSLPSQYITGLAEHLSPLMLSTSWTRITLWNRDLAPTPGAN |
| | LYGSHPFYLALEDGGSAHGVFLLNSNAMDVVLQPSPALSWRSTGGILDVYIFLGPEPKSVVQQYLDVVGYPFMPPYWGLGFHLCRWGYSSTAITRQVVENMTRAHFPLDVQWNDLDYMDSRRDFTFNKDGFRDFPAMVQELHQGG |
| | RRYMMIVDPAISSSGPAGSYRPYDEGLRRGVFITNETGQPLIGKVWPGSTAFPDFTNPTALAWWEDMVAEFHDQVPFDGMWIDMNEPSNFIRGSEDGCPNNELENPPYVPGVVGGTLQAATICASSHQFLSTHYNLHNLYGLTEA |
| | IASHRALVKARGTRPFVISRSTFAGHGRYAGHWTGDVWSSWEQLASSVPEILQFNLLGVPLVGADVCGFLGNTSEELCVRWTQLGAFYPFMRNHNSLLSLPQEPYSFSEPAQQAMRKALTLRYALLPHLYTLFHQAHVAGETVAR |
| | PLFLEFPKDSSTWTVDHQLLWGEALLITPVLQAGKAEVTGYFPLGTWYDLQTVPIEALGSLPPPPAAPREPAIHSEGQWVTLPAPLDTINVHLRAGYIIPLQGPGLTTTESRQQPMALAVALTKGGEARGELFWDDGESLEVLER |
| | GAYTQVIFLARNNTIVNELVRVTSEGAGLQLQKVTVLGVATAPQQVLSNGVPVSNFTYSPDTKVLDICVSLLMGEQFLVSWC |
| 108 | >mod GAA w/201 signal peptide |
| | MEFGLSWVFLVALLKGVQCEGHILLHDFLLVPRELSGSSPVLEETHPAHQQGASRPGPRDAQAHPGRPRAVPTQCDVPPNSRFDCAPDKAITQEQCEARGCCYIPAKQGLQGAQMGQPWCFFPPSYPSYKLENLSSSEMGYTATL |
| | TRTTPTFFPKDILTLRLDVMMETENRLHFTIKDPANRRYEVPLETPRVHSRAPSPLYSVEFSEEPFGVIVHRQLDGRVLLNTTVAPLFFADQFLQLSTSLPSQYITGLAEHLSPLMLSTSWTRITLWNRDLAPTPGANLYGSHPF |
| | YLALEDGGSAHGVFLLNSNAMDVVLQPSPALSWRSTGGILDVYIFLGPEPKSVVQQYLDVVGYPFMPPYWGLGFHLCRWGYSSTAITRQVVENMTRAHFPLDVQWNDLDYMDSRRDFTFNKDGFRDFPAMVQELHQGGRRYMMIV |
| | DPAISSSGPAGSYRPYDEGLRRGVFITNETGQPLIGKVWPGSTAFPDFTNPTALAWWEDMVAEFHDQVPFDGMWIDMNEPSNFIRGSEDGCPNNELENPPYVPGVVGGTLQAATICASSHQFLSTHYNLHNLYGLTEAIASHRAL |
| | VKARGTRPFVISRSTFAGHGRYAGHWTGDVWSSWEQLASSVPEILQFNLLGVPLVGADVCGFLGNTSEELCVRWTQLGAFYPFMRNHNSLLSLPQEPYSESEPAQQAMRKALTLRYALLPHLYTLFHQAHVAGETVARPLFLEFP |
| | KDSSTWTVDHQLLWGEALLITPVLQAGKAEVTGYFPLGTWYDLQTVPIEALGSLPPPPAAPREPAIHSEGQWVTLPAPLDTINVHLRAGYIIPLQGPGLTTTESRQQPMALAVALTKGGEARGELFWDDGESLEVLERGAYTQVI |
| | FLARNNTIVNELVRVTSEGAGLQLQKVTVLGVATAPQQVLSNGVPVSNFTYSPDTKVLDICVSLLMGEQFLVSWC |
| 109 | >mod GAA w/wt IL2 signal peptide |
| | MYRMQLLSCIALSLALVTNSGHILLHDFLLVPRELSGSSPVLEETHPAHQQGASRPGPRDAQAHPGRPRAVPTQCDVPPNSRFDCAPDKAITQEQCEARGCCYIPAKQGLQGAQMGQPWCFFPPSYPSYKLENLSSSEMGYTATL |
| | TRTTPTFFPKDILTLRLDVMMETENRLHFTIKDPANRRYEVPLETPRVHSRAPSPLYSVEFSEEPFGVIVHRQLDGRVLLNTTVAPLFFADQFLQLSTSLPSQYITGLAEHLSPLMLSTSWTRITLWNRDLAPTPGANLYGSHPF |
| | YLALEDGGSAHGVFLLNSNAMDVVLQPSPALSWRSTGGILDVYIFLGPEPKSVVQQYLDVVGYPFMPPYWGLGFHLCRWGYSSTAITRQVVENMTRAHFPLDVQWNDLDYMDSRRDFTFNKDGFRDFPAMVQELHQGGRRYMMIV |
| | DPAISSSGPAGSYRPYDEGLRRGVFITNETGQPLIGKVWPGSTAFPDFTNPTALAWWEDMVAEFHDQVPFDGMWIDMNEPSNFIRGSEDGCPNNELENPPYVPGVVGGTLQAATICASSHQFLSTHYNLHNLYGLTEAIASHRAL |
| | VKARGTRPFVISRSTFAGHGRYAGHWTGDVWSSWEQLASSVPEILQFNLLGVPLVGADVCGFLGNTSEELCVRWTQLGAFYPFMRNHNSLLSLPQEPYSFSEPAQQAMRKALTLRYALLPHLYTLFHQAHVAGETVARPLFLEFP |
| | KDSSTWTVDHQLLWGEALLITPVLQAGKAEVTGYFPLGTWYDLQTVPIEALGSLPPPPAAPREPAIHSEGQWVTLPAPLDTINVHLRAGYIIPLQGPGLTTTESRQQPMALAVALTKGGEARGELFWDDGESLEVLERGAYTQVI |
| | FLARNNTIVNELVRVTSEGAGLQLQKVTVLGVATAPQQVLSNGVPVSNFTYSPDTKVLDICVSLLMGEQFLVSWC |
| 110 | >mod GAA w/mut IL2 signal peptide |
| | MYRMQLLLLIALSLALVINSGHILLHDFLLVPRELSGSSPVLEETHPAHQQGASRPGPRDAQAHPGRPRAVPTQCDVPPNSRFDCAPDKAITQEQCEARGCCYIPAKQGLQGAQMGQPWCFFPPSYPSYKLENLSSSEMGYTATL |
| | TRTTPTFFPKDILTLRLDVMMETENRLHFTIKDPANRRYEVPLETPRVHSRAPSPLYSVEFSEEPFGVIVHRQLDGRVLLNTTVAPLFFADQFLQLSTSLPSQYITGLAEHLSPLMLSTSWTRITLWNRDLAPTPGANLYGSHPF |
| | YLALEDGGSAHGVFLLNSNAMDVVLQPSPALSWRSTGGILDVYIFLGPEPKSVVQQYLDVVGYPFMPPYWGLGFHLCRWGYSSTAITRQVVENMTRAHFPLDVQWNDLDYMDSRRDFTFNKDGFRDFPAMVQELHQGGRRYMMIV |
| | DPAISSSGPAGSYRPYDEGLRRGVFITNETGQPLIGKVWPGSTAFPDFTNPTALAWWEDMVAEFHDQVPFDGMWIDMNEPSNFIRGSEDGCPNNELENPPYVPGVVGGTLQAATICASSHQFLSTHYNLHNLYGLTEAIASHRAL |
| | VKARGTRPFVISRSTFAGHGRYAGHWTGDVWSSWEQLASSVPEILQFNLLGVPLVGADVCGFLGNTSEELCVRWTQLGAFYPFMRNHNSLLSLPQEPYSFSEPAQQAMRKALTLRYALLPHLYTLFHQAHVAGETVARPLFLEFP |
| | KDSSTWTVDHQLLWGEALLITPVLQAGKAEVTGYFPLGTWYDLQTVPIEALGSLPPPPAAPREPAIHSEGQWVTLPAPLDTINVHLRAGYIIPLQGPGLTTTESRQQPMALAVALTKGGEARGELFWDDGESLEVLERGAYTQVI |
| | FLARNNTIVNELVRVTSEGAGLQLQKVTVLGVATAPQQVLSNGVPVSNFTYSPDTKVLDICVSLLMGEQFLVSWC |
| 111 | >codon optimized GAA w/ codon optimized GAA signal peptide |
| | ATGGGAGTGAGGCACCCGCCCTGCTCCCACCGGCTCCTGGCCGTCTGCGCCCTCGTGTCCTTGGCAACCGCTGCACTCCTGGGGCACATCCTACTCCATGATTTCCTGCTGGTTCCCCGAGAGCTGAGTGGCTCCTCCCCAGTCC |
| | TGGAGGAGACTCACCCAGCTCACCAGCAGGGAGCCAGCAGACCAGGGCCCCGGGACGCCCAGGCACACCCCGGCCGTCCCAGAGCAGTGCCCACACAGTGCGACGTCCCCCCCAACAGCCGCTTCGATTGCGCCCCTGACAAGGC |
| | CATCACCCAGGAACAGTGCGAGGCCCGCGGCTGCTGCTACATCCCTGCAAAGCAGGGGCTGCAGGGAGCCCAGATGGGGCAGCCCTGGTGCTTCTTCCCACCCAGCTACCCCAGCTACAAGCTGGAGAACCTGAGCTCCTCTGAA |
| | ATGGGCTACACGGCCACCCTGACCCGTACCACCCCCACCTTCTTCCCCAAGGACATCCTGACCCTGCGGCTGGACGTGATGATGGAGACTGAGAACCGCCTCCACTTCACGATCAAAGATCCAGCTAACAGGCGCTACGAGGTGC |
| | CCTTGGAGACCCCGCGTGTCCACAGCCGGGCACCGTCCCCACTCTACAGCGTGGAGTTCTCCGAGGAGCCCTTCGGGGTGATCGTGCACCGGCAGCTGGACGGCCGCGTGCTGCTGAACACGACGGTGGCGCCCCTGTTCTTTGC |
| | GGACCAGTTCCTTCAGCTGTCCACCTCGCTGCCCTCGCAGTATATCACAGGCCTCGCCGAGCACCTCAGTCCCCTGATGCTCAGCACCAGCTGGACCAGGATCACCCTGTGGAACCGGGACCTTGCGCCCACGCCCGGTGCGAAC |
| | CTCTACGGGTCTCACCCTTTCTACCTGGCGCTGGAGGACGGCGGGTCGGCACACGGGGTGTTCCTGCTAAACAGCAACGCCATGGACGTGGTCCTGCAGCCGAGCCCTGCCCTTAGCTGGAGGTCGACAGGTGGGATCCTGGACG |
| | TCTACATCTTCCTGGGCCCAGAGCCCAAGAGCGTGGTGCAGCAGTACCTGGACGTGGTGGGATACCCGTTCATGCCGCCATACTGGGGCCTGGGCTTCCACCTGTGCCGCTGGGGCTACTCCTCCACCGCTATCACCCGCCAGGT |
| | GGTGGAGAACATGACCAGGGCCCACTTCCCCCTGGACGTCCAGTGGAACGATCTGGACTACATGGACTCCCGGAGGGACTTCACGTTCAACAAGGATGGCTTCCGGGACTTCCCGGCCATGGTGCAGGAGCTGCACCAGGGCGGC |
| | CGGCGCTACATGATGATCGTGGATCCTGCCATCAGCAGCTCGGGCCCTGCCGGGAGCTACAGGCCCTACGACGAGGGTCTGCGGAGGGGGGTTTTCATCACCAACGAGACCGGCCAGCCGCTGATTGGGAAGGTGTGGCCCGGGT |
| | CCACTGCCTTCCCCGACTTCACCAACCCCACAGCCCTGGCCTGGTGGGAGGACATGGTGGCTGAGTTCCATGACCAGGTGCCCTTCGACGGCATGTGGATTGACATGAACGAGCCTTCCAACTTCATCAGAGGCTCTGAGGACGG |
| | CTGCCCCAACAACGAGCTGGAGAACCCACCCTACGTGCCTGGGGTGGTTGGGGGGACCCTCCAGGCGGCCACCATCTGTGCCTCCAGCCACCAGTTTCTCTCCACACACTACAACCTGCACAACCTCTACGGCCTGACCGAAGCC |
| | ATCGCCTCCCACAGGGCGCTGGTGAAGGCTCGGGGGACACGCCCATTTGTGATCTCCCGCTCGACCTTTGCTGGCCACGGCCGATACGCCGGCCACTGGACGGGGGACGTGTGGAGCTCCTGGGAGCAGCTCGCCTCCTCCGTGC |
| | CAGAAATCCTGCAGTTTAACCTGCTGGGGGTGCCTCTGGTCGGGGCCGACGTCTGCGGCTTCCTGGGCAACACCTCAGAGGAGCTGTGTGTGCGCTGGACCCAGCTGGGGGCCTTCTACCCCTTCATGCGGAACCACAACAGCCT |
| | GCTCAGTCTGCCCCAGGAGCCGTACAGCTTCAGCGAGCCGGCCCAGCAGGCCATGAGGAAGGCCCTCACCCTGCGCTACGCACTCCTCCCCCACCTCTACACACTGTTCCACCAGGCCCACGTCGCGGGGGAGACCGTGGCCCGG |
| | CCCCTCTTCCTGGAGTTCCCCAAGGACTCTAGCACCTGGACTGTGGACCACCAGCTCCTGTGGGGGGAGGCCCTGCTCATCACCCCAGTGCTCCAGGCCGGGAAGGCCGAAGTGACTGGCTACTTCCCCTTGGGCACATGGTACG |
| | ACCTGCAGACGGTGCCAATAGAGGCCCTTGGCAGCCTCCCACCCCCACCTGCAGCTCCCCGTGAGCCAGCCATCCACAGCGAGGGGCAGTGGGTGACGCTGCCGGCCCCCCTGGACACCATCAACGTGCACCTCCGGGCTGGGTA |
| | CATCATCCCCCTGCAGGGCCCTGGCCTCACAACCACAGAGTCCCGCCAGCAGCCCATGGCCCTGGCTGTGGCCCTGACCAAGGGTGGAGAGGCCCGAGGGGAGCTGTTCTGGGACGACGGAGAGAGCCTGGAAGTGCTGGAGCGA |
| | GGGGCCTACACACAGGTCATCTTCCTGGCCAGGAATAACACGATCGTGAATGAGCTGGTACGTGTGACCAGTGAGGGAGCTGGCCTGCAGCTGCAGAAGGTGACTGTCCTGGGCGTGGCCACGGCGCCCCAGCAGGTCCTCTCCA |
| | ACGGTGTCCCTGTCTCCAACTTCACCTACAGCCCCGACACCAAGGTCCTGGACATCTGTGTCTCGCTGTTGATGGGAGAGCAGTTTCTCGTCAGCTGGTGTTGATAACTCGAG |
| 112 | > M2 pJAL130_dbDNA_MYO_GAA_CUP_Stop |
| | TCTAGAGCTAGCATATGGATCCATCGATTTAGGGATAACAGGGTAATTATCAGCACACAATTGCCCATTATACGCGCGTATAATGGACTATTGTGTGCTGATATCTGTACACTTAAGGGCTAGATCTTAGCTTACGTCACTAGAG |
| | GGTCCACGTTTAGTTTTTAAGATCCATTGATCTCCTAAACGCTGCAAGATTCGCAACCTGGTATACTTAGCCTAGGCGCTAGGTCCTAGTGCAGCGGGACTTTTTTTCTAAAGTCGTTGAGAGGAGGAGTCGTCAGACCAGATAG |
| | CTTTGATGTCCTGATCGGAAGGATCGTTGGCCCCCCTGCAGGCAGCTGTTAATTAACTGCGCGCTCGCTCGCTCACTGAGGCCGCCCGGGCAAAGCCCGGGCGTCGGGCGACCTTTGGTCGCCCGGCCTCAGTGAGCGAGCGAGC |
| | GCGCAGAGAGGGAGTGGCCAACTCCATCACTAGGGGTTCCTTGTAGTTAATGATTAACCCGCCATGCTACTTATCTACGGCGCGCCACGCGTTCTAGGAGTTAATTTTTAAAAAGCAGTCAAAAGTCCAAGTGCCCTTGCGAGCA |
| | TTTACTCTCTCTGTTTGCTCTGGTTAATAATCTCAGGAGCACAAACATTCCTTACTAGTTCTAGGAGTTAATTTTTAAAAAGCAGTCAAAAGTCCAAGTGCCCTTGCGAGCATTTACTCTCTCTGTTTGCTCTGGTTAATAATCT |
| | CAGGAGCACAAACATTCCTTACTAGTTCTAGAGCGGCCGCCAGTGTGCTGGAATTCGGCTTTTTTAGGGCTGGAAGCTACCTTTGACATCATTTCCTCTGCGAATGCATGTATAATTTCTACAGAACCTATTAGAAAGGATCACC |
| | CAGCCTCTGCTTTTGTACAACTTTCCCTTAAAAAACTGCCAATCCCACTGCTGTTTGGCCCAATAGTGAGAACTTTTTCCTGCTGCCTCTTGGTGCTTTTGCCTATGGCCCCTATTCTGCCTGCTGAAGACACTCTTGCCAGCAT |
| | GGACTTAAACCCCTCCAGCTCTGACAATCCTCTTTCTCTTTTGTTTTACATGAAGGGTCTGGCAGCCAAAGCAATCACTCAAAGTTCAAACCTTATCATTTTTTGCTTTGTTCCTCTTGGCCTTGGTTTTGTACATCAGCTGTGA |
| | AAATACCATCCCAGGGTATTTATAGCTGGGGTTAATTTATAACTGAGAGTGCTCTAGTTCTGCAATACAGGACATGCTATAAAAATGGAAAGATGTTGCTTTCTGAGAGATCAGCTTACATGTGCTAGCGTAGCCAACCATGGGA |
| | GTGAGGCACCCGCCCTGCTCCCACCGGCTCCTGGCCGTCTGCGCCCTCGTGTCCTTGGCAACCGCTGCACTCCTGGGGCACATCCTACTCCATGATTTCCTGCTGGTTCCCCGAGAGCTGAGTGGCTCCTCCCCAGTCCTGGAGG |
| | AGACTCACCCAGCTCACCAGCAGGGAGCCAGCAGACCAGGGCCCCGGGACGCCCAGGCACACCCCGGCCGTCCCAGAGCAGTGCCCACACAGTGCGACGTCCCCCCCAACAGCCGCTTCGATTGCGCCCCTGACAAGGCCATCAC |
| | CCAGGAACAGTGCGAGGCCCGCGGCTGCTGCTACATCCCTGCAAAGCAGGGGCTGCAGGGAGCCCAGATGGGGCAGCCCTGGTGCTTCTTCCCACCCAGCTACCCCAGCTACAAGCTGGAGAACCTGAGCTCCTCTGAAATGGGC |
| | TACACGGCCACCCTGACCCGTACCACCCCCACCTTCTTCCCCAAGGACATCCTGACCCTGCGGCTGGACGTGATGATGGAGACTGAGAACCGCCTCCACTTCACGATCAAAGATCCAGCTAACAGGCGCTACGAGGTGCCCTTGG |
| | AGACCCCGCGTGTCCACAGCCGGGCACCGTCCCCACTCTACAGCGTGGAGTTCTCCGAGGAGCCCTTCGGGGTGATCGTGCACCGGCAGCTGGACGGCCGCGTGCTGCTGAACACGACGGTGGCGCCCCTGTTCTTTGCGGACCA |
| | GTTCCTTCAGCTGTCCACCTCGCTGCCCTCGCAGTATATCACAGGCCTCGCCGAGCACCTCAGTCCCCTGATGCTCAGCACCAGCTGGACCAGGATCACCCTGTGGAACCGGGACCTTGCGCCCACGCCCGGTGCGAACCTCTAC |
| | GGGTCTCACCCTTTCTACCTGGCGCTGGAGGACGGCGGGTCGGCACACGGGGTGTTCCTGCTAAACAGCAACGCCATGGACGTGGTCCTGCAGCCGAGCCCTGCCCTTAGCTGGAGGTCGACAGGTGGGATCCTGGACGTCTACA |
| | TCTTCCTGGGCCCAGAGCCCAAGAGCGTGGTGCAGCAGTACCTGGACGTGGTGGGATACCCGTTCATGCCGCCATACTGGGGCCTGGGCTTCCACCTGTGCCGCTGGGGCTACTCCTCCACCGCTATCACCCGCCAGGTGGTGGA |
| | GAACATGACCAGGGCCCACTTCCCCCTGGACGTCCAGTGGAACGATCTGGACTACATGGACTCCCGGAGGGACTTCACGTTCAACAAGGATGGCTTCCGGGACTTCCCGGCCATGGTGCAGGAGCTGCACCAGGGCGGCCGGCGC |
| | TACATGATGATCGTGGATCCTGCCATCAGCAGCTCGGGCCCTGCCGGGAGCTACAGGCCCTACGACGAGGGTCTGCGGAGGGGGGTTTTCATCACCAACGAGACCGGCCAGCCGCTGATTGGGAAGGTGTGGCCCGGGTCCACTG |
| | CCTTCCCCGACTTCACCAACCCCACAGCCCTGGCCTGGTGGGAGGACATGGTGGCTGAGTTCCATGACCAGGTGCCCTTCGACGGCATGTGGATTGACATGAACGAGCCTTCCAACTTCATCAGAGGCTCTGAGGACGGCTGCCC |
| | CAACAACGAGCTGGAGAACCCACCCTACGTGCCTGGGGTGGTTGGGGGGACCCTCCAGGCGGCCACCATCTGTGCCTCCAGCCACCAGTTTCTCTCCACACACTACAACCTGCACAACCTCTACGGCCTGACCGAAGCCATCGCC |
| | TCCCACAGGGCGCTGGTGAAGGCTCGGGGGACACGCCCATTTGTGATCTCCCGCTCGACCTTTGCTGGCCACGGCCGATACGCCGGCCACTGGACGGGGGACGTGTGGAGCTCCTGGGAGCAGCTCGCCTCCTCCGTGCCAGAAA |
| | TCCTGCAGTTTAACCTGCTGGGGGTGCCTCTGGTCGGGGCCGACGTCTGCGGCTTCCTGGGCAACACCTCAGAGGAGCTGTGTGTGCGCTGGACCCAGCTGGGGGCCTTCTACCCCTTCATGCGGAACCACAACAGCCTGCTCAG |
| | TCTGCCCCAGGAGCCGTACAGCTTCAGCGAGCCGGCCCAGCAGGCCATGAGGAAGGCCCTCACCCTGCGCTACGCACTCCTCCCCCACCTCTACACACTGTTCCACCAGGCCCACGTCGCGGGGGAGACCGTGGCCCGGCCCCTC |
| | TTCCTGGAGTTCCCCAAGGACTCTAGCACCTGGACTGTGGACCACCAGCTCCTGTGGGGGGAGGCCCTGCTCATCACCCCAGTGCTCCAGGCCGGGAAGGCCGAAGTGACTGGCTACTTCCCCTTGGGCACATGGTACGACCTGC |
| | AGACGGTGCCAATAGAGGCCCTTGGCAGCCTCCCACCCCCACCTGCAGCTCCCCGTGAGCCAGCCATCCACAGCGAGGGGCAGTGGGTGACGCTGCCGGCCCCCCTGGACACCATCAACGTGCACCTCCGGGCTGGGTACATCAT |
| | CCCCCTGCAGGGCCCTGGCCTCACAACCACAGAGTCCCGCCAGCAGCCCATGGCCCTGGCTGTGGCCCTGACCAAGGGTGGAGAGGCCCGAGGGGAGCTGTTCTGGGACGACGGAGAGAGCCTGGAAGTGCTGGAGCGAGGGGCC |
| | TACACACAGGTCATCTTCCTGGCCAGGAATAACACGATCGTGAATGAGCTGGTACGTGTGACCAGTGAGGGAGCTGGCCTGCAGCTGCAGAAGGTGACTGTCCTGGGCGTGGCCACGGCGCCCCAGCAGGTCCTCTCCAACGGTG |
| | TCCCTGTCTCCAACTTCACCTACAGCCCCGACACCAAGGTCCTGGACATCTGTGTCTCGCTGTTGATGGGAGAGCAGTTTCTCGTCAGCTGGTGTTGATAACTCGAGCCGGGCGGAGTGTGTTAGTCTCTCCAGAGGGAGGCTGG |
| | TTCCCCAGGGAAGCAGAGCCTGTGTGCGGGCAGCAGCTGTGTGCGGGCCTGGGGGTTGTTAAGTGCAATTATTTTTAATAAAAGGGGCATTTGGAAAAAAAAAAAAAAGGTAGCAGTCGACAGATGAATTCTGCAGATCTGTGGC |
| | TTCTAGCTGCCCGGGTGGCATCCCTGTGACCCCTCCCCAGTGCCTCTCCTGGCCCTGGAAGTTGCCACTCCAGTGCCCACCAGCCTTGTCCTAATAAAATTAAGTTGCATCATTTTGTCTGACTAGGTGTCCTTCTATAATATTA |
| | TGGGGTGGAGGGGGGTGGTATGGAGCAAGGGGCAAGTTGGGAAGACAACCTGTAGGGCCTGCGGGGTCTATTGGGAACCAAGCTGGAGTGCAGTGGCACAATCTTGGCTCACTGCAATCTCCGCCTCCTGGGTTCAAGCGATTCT |
| | CCTGCCTCAGCCTCCCGAGTTGTTGGGATTCCAGGCATGCATGACCAGGCTCAGCTAATTTTTGTTTTTTTGGTAGAGACGGGGTTTCACCATATTGGCCAGGCTGGTCTCCAACTCCTAATCTCAGGTGATCTACCCACCTTGG |
| | CCTCCCAAATTGCTGGGATTACAGGCGTGAACCACTGCTCCCTTCCCTGTCCTTCTGATTTTAAAAAAAAGAGAAATGTTCTGGCACCTGCACTTGCACTGGGGACAGCCTATTTTGCTAGTTTGTTTTGTTTCGTTTTGTTTTG |
| | ATGGAGAGCGTATGTTGTTTAAACGCGGCCGCGTAGATAAGTAGCATGGCGGGTTAATCATTAACTACAAGGAACCCCTAGTGATGGAGTTGGCCACTCCCTCTCTGCGCGCTCGCTCGCTCACTGAGGCCGGGCGACCAAAGGT |
| | CGCCCGACGCCCGGGCTTTGCCCGGGCGGCCTCAGTGAGCGAGCGAGCGCGCAGTTAATTAAGGCGCCCTAGGCCGACCCTTAGACTCTGTACTCAGTTCTATAAACGAGCCATTGGATACGAGATCCGTAGATTGATAAGGGAC |
| | ACGGAATATCCCCGGACGCAATAGACACCGGTGGACAGCTTGGTATCCTGAGCACAGTCGCGCGTCCGAATCTAGCTCTACTTTAGAGGCCCCGGATTCTGATGGTCGTAGACCGCAGAACCGATTGGGGGGATGAGATCTACTA |
| | GTTATCAGCACACAATTGCCCATTATACGCGCGTATAATGGACTATTGTGTGCTGATATAGGGATAACAGGGTAATTCTAGAGCTAGCATATGGATCCATCGATTTGATGCGGTATTTTCTCCTTACGCATCTGTGCGGTATTTC |
| | ACACCGCATACGTCAAAGCAACCATAGTACGCGCCCTGTAGCGGCGCATTAAGCGCGGCGGGTGTGGTGGTTACGCGCAGCGTGACCGCTACACTTGCCAGCGCCCTAGCGCCCGCTCCTTTCGCTTTCTTCCCTTCCTTTCTCG |
| | CCACGTTCGCCGGCTTTCCCCGTCAAGCTCTAAATCGGGGGCTCCCTTTAGGGTTCCGATTTAGTGCTTTACGGCACCTCGACCCCAAAAAACTTGATTTGGGTGATGGTTCACGTAGTGGGCCATCGCCCTGATAGACGGTTTT |
| | TCGCCCTTTGACGTTGGAGTCCACGTTCTTTAATAGTGGACTCTTGTTCCAAACTGGAACAACACTCAACTCTATCTCGGGCTATTCTTTTGATTTATAAGGGATTTTGCCGATTTCGGTCTATTGGTTAAAAAATGAGCTGATT |
| | TAACAAAAATTTAACGCGAATTTTAACAAAATATTAACGTTTACAATTTTATGGTGCACTCTCAGTACAATCTGCTCTGATGCCGCATAGTTAAGCCAGCCCCGACACCCGCCAACACCCGCTGACGCGCCCTGACGGGCTTGTC |
| | TGCTCCCGGCATCCGCTTACAGACAAGCTGTGACCGTCTCCGGGAGCTGCATGTGTCAGAGGTTTTCACCGTCATCACCGAAACGCGCGAGACGAAAGGGCCTCGTGATACGCCTATTTTTATAGGTTAATGTCATGATAATAAT |
| | GGTTTCTTAGACGTCAGGTGGCACTTTTCGGGGAAATGTGCGCGGAACCCCTATTTGTTTATTTTTCTAAATACATTCAAATATGTATCCGCTCATGAGACAATAACCCTGATAAATGCTTCAATAATATTGAAAAAGGAAGAGT |
| | ATGAGCCATATTCAACGGGAAACGTCGAGGCCGCGATTAAATTCCAACATGGATGCTGATTTATATGGGTATAAATGGGCTCGCGATAATGTCGGGCAATCAGGTGCGACAATCTATCGCTTGTATGGGAAGCCCGATGCGCCAG |
| | AGTTGTTTCTGAAACATGGCAAAGGTAGCGTTGCCAATGATGTTACAGATGAGATGGTCAGACTAAACTGGCTGACGGAATTTATGCCTCTTCCGACCATCAAGCATTTTATCCGTACTCCTGATGATGCATGGTTACTCACCAC |
| | TGCGATCCCCGGAAAAACAGCATTCCAGGTATTAGAAGAATATCCTGATTCAGGTGAAAATATTGTTGATGCGCTGGCAGTGTTCCTGCGCCGGTTGCATTCGATTCCTGTTTGTAATTGTCCTTTTAACAGCGATCGCGTATTT |
| | CGTCTCGCTCAGGCGCAATCACGAATGAATAACGGTTTGGTTGATGCGAGTGATTTTGATGACGAGCGTAATGGCTGGCCTGTTGAACAAGTCTGGAAAGAAATGCATAAACTTTTGCCATTCTCACCGGATTCAGTCGTCACTC |
| | ATGGTGATTTCTCACTTGATAACCTTATTTTTGACGAGGGGAAATTAATAGGTTGTATTGATGTTGGACGAGTCGGAATCGCAGACCGATACCAGGATCTTGCCATCCTATGGAACTGCCTCGGTGAGTTTTCTCCTTCATTACA |
| | GAAACGGCTTTTTCAAAAATATGGTATTGATAATCCTGATATGAATAAATTGCAGTTTCATTTGATGCTCGATGAGTTTTTCTAAGCGTATAATGGTCTAGAGCTAGCATATGGATCCATCGATTCCATTATACGCCTGTCAGAC |
| | CAAGTTTACTCATATATACTTTAGATTGATTTAAAACTTCATTTTTAATTTAAAAGGATCTAGGTGAAGATCCTTTTTGATAATCTCATGACCAAAATCCCTTAACGTGAGTTTTCGTTCCACTGAGCGTCAGACCCCGTAGAAA |
| | AGATCAAAGGATCTTCTTGAGATCCTTTTTTTCTGCGCGTAATCTGCTGCTTGCAAACAAAAAAACCACCGCTACCAGCGGTGGTTTGTTTGCCGGATCAAGAGCTACCAACTCTTTTTCCGAAGGTAACTGGCTTCAGCAGAGC |
| | GCAGATACCAAATACTGTTCTTCTAGTGTAGCCGTAGTTAGGCCACCACTTCAAGAACTCTGTAGCACCGCCTACATACCTCGCTCTGCTAATCCTGTTACCAGTGGCTGCTGCCAGTGGCGATAAGTCGTGTCTTACCGGGTTG |
| | GACTCAAGACGATAGTTACCGGATAAGGCGCAGCGGTCGGGCTGAACGGGGGGTTCGTGCACACAGCCCAGCTTGGAGCGAACGACCTACACCGAACTGAGATACCTACAGCGTGAGCTATGAGAAAGCGCCACGCTTCCCGAAG |
| | GGAGAAAGGCGGACAGGTATCCGGTAAGCGGCAGGGTCGGAACAGGAGAGCGCACGAGGGAGCTTCCAGGGGGAAACGCCTGGTATCTTTATAGTCCTGTCGGGTTTCGCCACCTCTGACTTGAGCGTCGATTTTTGTGATGCTC |
| | GTCAGGGGGGCGGAGCCTATGGAAAAACGCCAGCAACGCGGCCTTTTTACGGTTCCTGGCCTTTTGCTGGCCTTTTGCTCACATGT |
| 113 | > M3 pJAL130_dbDNA MYO_GAA_CUP_Stop |
| | TCTAGAGCTAGCATATGGATCCATCGATTTAGGGATAACAGGGTAATTATCAGCACACAATTGCCCATTATACGCGCGTATAATGGACTATTGTGTGCTGATATCTGTACACTTAAGGGCTAGATCTTAGCTTACGTCACTAGAG |
| | GGTCCACGTTTAGTTTTTAAGATCCATTGATCTCCTAAACGCTGCAAGATTCGCAACCTGGTATACTTAGCCTAGGCGCTAGGTCCTAGTGCAGCGGGACTTTTTTTCTAAAGTCGTTGAGAGGAGGAGTCGTCAGACCAGATAG |
| | CTTTGATGTCCTGATCGGAAGGATCGTTGGCCCCCCTGCAGGCAGCTGTTAATTAACTGCGCGCTCGCTCGCTCACTGAGGCCGCCCGGGCAAAGCCCGGGCGTCGGGCGACCTTTGGTCGCCCGGCCTCAGTGAGCGAGCGAGC |
| | GCGCAGAGAGGGAGTGGCCAACTCCATCACTAGGGGTTCCTTGTAGTTAATGATTAACCCGCCATGCTACTTATCTACGGCGCGCCACGCGTTCTAGGAGTTAATTTTTAAAAAGCAGTCAAAAGTCCAAGTGCCCTTGCGAGCA |
| | TTTACTCTCTCTGTTTGCTCTGGTTAATAATCTCAGGAGCACAAACATTCCTTACTAGTTCTAGGAGTTAATTTTTAAAAAGCAGTCAAAAGTCCAAGTGCCCTTGCGAGCATTTACTCTCTCTGTTTGCTCTGGTTAATAATCT |
| | CAGGAGCACAAACATTCCTTACTAGTTCTAGAGCGGCCGCCAGTGTGCTGGAATTCGGCTTTTTTAGGGCTGGAAGCTACCTTTGACATCATTTCCTCTGCGAATGCATGTATAATTTCTACAGAACCTATTAGAAAGGATCACC |
| | CAGCCTCTGCTTTTGTACAACTTTCCCTTAAAAAACTGCCAATCCCACTGCTGTTTGGCCCAATAGTGAGAACTTTTTCCTGCTGCCTCTTGGTGCTTTTGCCTATGGCCCCTATTCTGCCTGCTGAAGACACTCTTGCCAGCAT |
| | GGACTTAAACCCCTCCAGCTCTGACAATCCTCTTTCTCTTTTGTTTTACATGAAGGGTCTGGCAGCCAAAGCAATCACTCAAAGTTCAAACCTTATCATTTTTTGCTTTGTTCCTCTTGGCCTTGGTTTTGTACATCAGCTTTGA |
| | AAATACCATCCCAGGGTTAATGCTGGGGTTAATTTATAACTGAGAGTGCTCTAGTTCTGCAATACAGGACATGCTATAAAAATGGAAAGATGTTGCTTTCTGAGAGATCAGCTTACATGTGCTAGCGTAGCCAACCATGGGAGTG |
| | AGGCACCCGCCCTGCTCCCACCGGCTCCTGGCCGTCTGCGCCCTCGTGTCCTTGGCAACCGCTGCACTCCTGGGGCACATCCTACTCCATGATTTCCTGCTGGTTCCCCGAGAGCTGAGTGGCTCCTCCCCAGTCCTGGAGGAGA |
| | CTCACCCAGCTCACCAGCAGGGAGCCAGCAGACCAGGGCCCCGGGACGCCCAGGCACACCCCGGCCGTCCCAGAGCAGTGCCCACACAGTGCGACGTCCCCCCCAACAGCCGCTTCGATTGCGCCCCTGACAAGGCCATCACCCA |
| | GGAACAGTGCGAGGCCCGCGGCTGCTGCTACATCCCTGCAAAGCAGGGGCTGCAGGGAGCCCAGATGGGGCAGCCCTGGTGCTTCTTCCCACCCAGCTACCCCAGCTACAAGCTGGAGAACCTGAGCTCCTCTGAAATGGGCTAC |
| | ACGGCCACCCTGACCCGTACCACCCCCACCTTCTTCCCCAAGGACATCCTGACCCTGCGGCTGGACGTGATGATGGAGACTGAGAACCGCCTCCACTTCACGATCAAAGATCCAGCTAACAGGCGCTACGAGGTGCCCTTGGAGA |
| | CCCCGCGTGTCCACAGCCGGGCACCGTCCCCACTCTACAGCGTGGAGTTCTCCGAGGAGCCCTTCGGGGTGATCGTGCACCGGCAGCTGGACGGCCGCGTGCTGCTGAACACGACGGTGGCGCCCCTGTTCTTTGCGGACCAGTT |
| | CCTTCAGCTGTCCACCTCGCTGCCCTCGCAGTATATCACAGGCCTCGCCGAGCACCTCAGTCCCCTGATGCTCAGCACCAGCTGGACCAGGATCACCCTGTGGAACCGGGACCTTGCGCCCACGCCCGGTGCGAACCTCTACGGG |
| | TCTCACCCTTTCTACCTGGCGCTGGAGGACGGCGGGTCGGCACACGGGGTGTTCCTGCTAAACAGCAACGCCATGGACGTGGTCCTGCAGCCGAGCCCTGCCCTTAGCTGGAGGTCGACAGGTGGGATCCTGGACGTCTACATCT |
| | TCCTGGGCCCAGAGCCCAAGAGCGTGGTGCAGCAGTACCTGGACGTGGTGGGATACCCGTTCATGCCGCCATACTGGGGCCTGGGCTTCCACCTGTGCCGCTGGGGCTACTCCTCCACCGCTATCACCCGCCAGGTGGTGGAGAA |
| | CATGACCAGGGCCCACTTCCCCCTGGACGTCCAGTGGAACGATCTGGACTACATGGACTCCCGGAGGGACTTCACGTTCAACAAGGATGGCTTCCGGGACTTCCCGGCCATGGTGCAGGAGCTGCACCAGGGCGGCCGGCGCTAC |
| | ATGATGATCGTGGATCCTGCCATCAGCAGCTCGGGCCCTGCCGGGAGCTACAGGCCCTACGACGAGGGTCTGCGGAGGGGGGTTTTCATCACCAACGAGACCGGCCAGCCGCTGATTGGGAAGGTGTGGCCCGGGTCCACTGCCT |
| | TCCCCGACTTCACCAACCCCACAGCCCTGGCCTGGTGGGAGGACATGGTGGCTGAGTTCCATGACCAGGTGCCCTTCGACGGCATGTGGATTGACATGAACGAGCCTTCCAACTTCATCAGAGGCTCTGAGGACGGCTGCCCCAA |
| | CAACGAGCTGGAGAACCCACCCTACGTGCCTGGGGTGGTTGGGGGGACCCTCCAGGCGGCCACCATCTGTGCCTCCAGCCACCAGTTTCTCTCCACACACTACAACCTGCACAACCTCTACGGCCTGACCGAAGCCATCGCCTCC |
| | CACAGGGCGCTGGTGAAGGCTCGGGGGACACGCCCATTTGTGATCTCCCGCTCGACCTTTGCTGGCCACGGCCGATACGCCGGCCACTGGACGGGGGACGTGTGGAGCTCCTGGGAGCAGCTCGCCTCCTCCGTGCCAGAAATCC |
| | TGCAGTTTAACCTGCTGGGGGTGCCTCTGGTCGGGGCCGACGTCTGCGGCTTCCTGGGCAACACCTCAGAGGAGCTGTGTGTGCGCTGGACCCAGCTGGGGGCCTTCTACCCCTTCATGCGGAACCACAACAGCCTGCTCAGTCT |
| | GCCCCAGGAGCCGTACAGCTTCAGCGAGCCGGCCCAGCAGGCCATGAGGAAGGCCCTCACCCTGCGCTACGCACTCCTCCCCCACCTCTACACACTGTTCCACCAGGCCCACGTCGCGGGGGAGACCGTGGCCCGGCCCCTCTTC |
| | CTGGAGTTCCCCAAGGACTCTAGCACCTGGACTGTGGACCACCAGCTCCTGTGGGGGGAGGCCCTGCTCATCACCCCAGTGCTCCAGGCCGGGAAGGCCGAAGTGACTGGCTACTTCCCCTTGGGCACATGGTACGACCTGCAGA |
| | CGGTGCCAATAGAGGCCCTTGGCAGCCTCCCACCCCCACCTGCAGCTCCCCGTGAGCCAGCCATCCACAGCGAGGGGCAGTGGGTGACGCTGCCGGCCCCCCTGGACACCATCAACGTGCACCTCCGGGCTGGGTACATCATCCC |
| | CCTGCAGGGCCCTGGCCTCACAACCACAGAGTCCCGCCAGCAGCCCATGGCCCTGGCTGTGGCCCTGACCAAGGGTGGAGAGGCCCGAGGGGAGCTGTTCTGGGACGACGGAGAGAGCCTGGAAGTGCTGGAGCGAGGGGCCTAC |
| | ACACAGGTCATCTTCCTGGCCAGGAATAACACGATCGTGAATGAGCTGGTACGTGTGACCAGTGAGGGAGCTGGCCTGCAGCTGCAGAAGGTGACTGTCCTGGGCGTGGCCACGGCGCCCCAGCAGGTCCTCTCCAACGGTGTCC |
| | CTGTCTCCAACTTCACCTACAGCCCCGACACCAAGGTCCTGGACATCTGTGTCTCGCTGTTGATGGGAGAGCAGTTTCTCGTCAGCTGGTGTTGATAACTCGAGCCGGGCGGAGTGTGTTAGTCTCTCCAGAGGGAGGCTGGTTC |
| | CCCAGGGAAGCAGAGCCTGTGTGCGGGCAGCAGCTGTGTGCGGGCCTGGGGGTTGTTAAGTGCAATTATTTTTAATAAAAGGGGCATTTGGAAAAAAAAAAAAAAGGTAGCAGTCGACAGATGAATTCTGCAGATCTGTGGCTTC |
| | TAGCTGCCCGGGTGGCATCCCTGTGACCCCTCCCCAGTGCCTCTCCTGGCCCTGGAAGTTGCCACTCCAGTGCCCACCAGCCTTGTCCTAATAAAATTAAGTTGCATCATTTTGTCTGACTAGGTGTCCTTCTATAATATTATGG |
| | GGTGGAGGGGGGTGGTATGGAGCAAGGGGCAAGTTGGGAAGACAACCTGTAGGGCCTGCGGGGTCTATTGGGAACCAAGCTGGAGTGCAGTGGCACAATCTTGGCTCACTGCAATCTCCGCCTCCTGGGTTCAAGCGATTCTCCT |
| | GCCTCAGCCTCCCGAGTTGTTGGGATTCCAGGCATGCATGACCAGGCTCAGCTAATTTTTGTTTTTTTGGTAGAGACGGGGTTTCACCATATTGGCCAGGCTGGTCTCCAACTCCTAATCTCAGGTGATCTACCCACCTTGGCCT |
| | CCCAAATTGCTGGGATTACAGGCGTGAACCACTGCTCCCTTCCCTGTCCTTCTGATTTTAAAAAAAAGAGAAATGTTCTGGCACCTGCACTTGCACTGGGGACAGCCTATTTTGCTAGTTTGTTTTGTTTCGTTTTGTTTTGATG |
| | GAGAGCGTATGTTGTTTAAACGCGGCCGCGTAGATAAGTAGCATGGCGGGTTAATCATTAACTACAAGGAACCCCTAGTGATGGAGTTGGCCACTCCCTCTCTGCGCGCTCGCTCGCTCACTGAGGCCGGGCGACCAAAGGTCGC |
| | CCGACGCCCGGGCTTTGCCCGGGCGGCCTCAGTGAGCGAGCGAGCGCGCAGTTAATTAAGGCGCCCTAGGCCGACCCTTAGACTCTGTACTCAGTTCTATAAACGAGCCATTGGATACGAGATCCGTAGATTGATAAGGGACACG |
| | GAATATCCCCGGACGCAATAGACACCGGTGGACAGCTTGGTATCCTGAGCACAGTCGCGCGTCCGAATCTAGCTCTACTTTAGAGGCCCCGGATTCTGATGGTCGTAGACCGCAGAACCGATTGGGGGGATGAGATCTACTAGTT |
| | ATCAGCACACAATTGCCCATTATACGCGCGTATAATGGACTATTGTGTGCTGATATAGGGATAACAGGGTAATTCTAGAGCTAGCATATGGATCCATCGATTTGATGCGGTATTTTCTCCTTACGCATCTGTGCGGTATTTCACA |
| | CCGCATACGTCAAAGCAACCATAGTACGCGCCCTGTAGCGGCGCATTAAGCGCGGCGGGTGTGGTGGTTACGCGCAGCGTGACCGCTACACTTGCCAGCGCCCTAGCGCCCGCTCCTTTCGCTTTCTTCCCTTCCTTTCTCGCCA |
| | CGTTCGCCGGCTTTCCCCGTCAAGCTCTAAATCGGGGGCTCCCTTTAGGGTTCCGATTTAGTGCTTTACGGCACCTCGACCCCAAAAAACTTGATTTGGGTGATGGTTCACGTAGTGGGCCATCGCCCTGATAGACGGTTTTTCG |
| | CCCTTTGACGTTGGAGTCCACGTTCTTTAATAGTGGACTCTTGTTCCAAACTGGAACAACACTCAACTCTATCTCGGGCTATTCTTTTGATTTATAAGGGATTTTGCCGATTTCGGTCTATTGGTTAAAAAATGAGCTGATTTAA |
| | CAAAAATTTAACGCGAATTTTAACAAAATATTAACGTTTACAATTTTATGGTGCACTCTCAGTACAATCTGCTCTGATGCCGCATAGTTAAGCCAGCCCCGACACCCGCCAACACCCGCTGACGCGCCCTGACGGGCTTGTCTGC |
| | TCCCGGCATCCGCTTACAGACAAGCTGTGACCGTCTCCGGGAGCTGCATGTGTCAGAGGTTTTCACCGTCATCACCGAAACGCGCGAGACGAAAGGGCCTCGTGATACGCCTATTTTTATAGGTTAATGTCATGATAATAATGGT |
| | TTCTTAGACGTCAGGTGGCACTTTTCGGGGAAATGTGCGCGGAACCCCTATTTGTTTATTTTTCTAAATACATTCAAATATGTATCCGCTCATGAGACAATAACCCTGATAAATGCTTCAATAATATTGAAAAAGGAAGAGTATG |
| | AGCCATATTCAACGGGAAACGTCGAGGCCGCGATTAAATTCCAACATGGATGCTGATTTATATGGGTATAAATGGGCTCGCGATAATGTCGGGCAATCAGGTGCGACAATCTATCGCTTGTATGGGAAGCCCGATGCGCCAGAGT |
| | TGTTTCTGAAACATGGCAAAGGTAGCGTTGCCAATGATGTTACAGATGAGATGGTCAGACTAAACTGGCTGACGGAATTTATGCCTCTTCCGACCATCAAGCATTTTATCCGTACTCCTGATGATGCATGGTTACTCACCACTGC |
| | GATCCCCGGAAAAACAGCATTCCAGGTATTAGAAGAATATCCTGATTCAGGTGAAAATATTGTTGATGCGCTGGCAGTGTTCCTGCGCCGGTTGCATTCGATTCCTGTTTGTAATTGTCCTTTTAACAGCGATCGCGTATTTCGT |
| | CTCGCTCAGGCGCAATCACGAATGAATAACGGTTTGGTTGATGCGAGTGATTTTGATGACGAGCGTAATGGCTGGCCTGTTGAACAAGTCTGGAAAGAAATGCATAAACTTTTGCCATTCTCACCGGATTCAGTCGTCACTCATG |
| | GTGATTTCTCACTTGATAACCTTATTTTTGACGAGGGGAAATTAATAGGTTGTATTGATGTTGGACGAGTCGGAATCGCAGACCGATACCAGGATCTTGCCATCCTATGGAACTGCCTCGGTGAGTTTTCTCCTTCATTACAGAA |
| | ACGGCTTTTTCAAAAATATGGTATTGATAATCCTGATATGAATAAATTGCAGTTTCATTTGATGCTCGATGAGTTTTTCTAAGCGTATAATGGTCTAGAGCTAGCATATGGATCCATCGATTCCATTATACGCCTGTCAGACCAA |
| | GTTTACTCATATATACTTTAGATTGATTTAAAACTTCATTTTTAATTTAAAAGGATCTAGGTGAAGATCCTTTTTGATAATCTCATGACCAAAATCCCTTAACGTGAGTTTTCGTTCCACTGAGCGTCAGACCCCGTAGAAAAGA |
| | TCAAAGGATCTTCTTGAGATCCTTTTTTTCTGCGCGTAATCTGCTGCTTGCAAACAAAAAAACCACCGCTACCAGCGGTGGTTTGTTTGCCGGATCAAGAGCTACCAACTCTTTTTCCGAAGGTAACTGGCTTCAGCAGAGCGCA |
| | GATACCAAATACTGTTCTTCTAGTGTAGCCGTAGTTAGGCCACCACTTCAAGAACTCTGTAGCACCGCCTACATACCTCGCTCTGCTAATCCTGTTACCAGTGGCTGCTGCCAGTGGCGATAAGTCGTGTCTTACCGGGTTGGAC |
| | TCAAGACGATAGTTACCGGATAAGGCGCAGCGGTCGGGCTGAACGGGGGGTTCGTGCACACAGCCCAGCTTGGAGCGAACGACCTACACCGAACTGAGATACCTACAGCGTGAGCTATGAGAAAGCGCCACGCTTCCCGAAGGGA |
| | GAAAGGCGGACAGGTATCCGGTAAGCGGCAGGGTCGGAACAGGAGAGCGCACGAGGGAGCTTCCAGGGGGAAACGCCTGGTATCTTTATAGTCCTGTCGGGTTTCGCCACCTCTGACTTGAGCGTCGATTTTTGTGATGCTCGTC |
| | AGGGGGGCGGAGCCTATGGAAAAACGCCAGCAACGCGGCCTTTTTACGGTTCCTGGCCTTTTGCTGGCCTTTTGCTCACATGT |
| 270 | > CRM_SP0107 |
| | CCCTGGAGAGTCCTTTAGCAGGGCAAAGTGCAACATAGGCAGACCTTAAGGGATGACTCAGTAACAGATAAGCTTTGTGTGCCTGCAGGGGGATGGGAAGAGGGTGGGGCAGGAGAGGGACATAAAAGGGCTCTGAGGCATTGTA |
| | CTGTGAATTCCTTCAGTCTCCTGTTTGGAGAAGACAGAGCCAATGAGGCCCTCGTTCCAGGGAAACAGAATATGCTCAGCATGACGCAGCACTCCCTGAACTTTCCGGTTACATCACCCAATAGCTGAGATCAGA |
| 271 | > CRM_SP0109 |
| | CCCTGGAGAGTCCTTTAGCAGGGCAAAGTGCAACATAGGCAGACCTTAAGGGATGACTCAGTAACAGATAAGCTTTGTGTGCCTGCAGGGGGATGGGAAGAGGGTGGGGCAGGAGAGGGACATAAAAGGGCTCTGAGGCATTGTA |
| | CTGTGAATTCCTTCAGTCTCCTGCTCTGCTCAGCCAGTCAGCCCTGCCTCCCTTGTTTAGGACCACACAGCACTGCTGGGTGTCTGCCTTTCCTTG |
| 273 | > CRM_SP0111 |
| | AGGTTAATTTTTAAAAAGCAGTCAAAAGTCCAAGTGGCCCTTGGCAGCATTTACTCTCTCTGTTTGCTCTGGTTAATAATCTCAGGAGCACAAACATTCCTTTGGAGAAGACAGAGCCAATGAGGCCCTCGTTCCAGGGAAACAG |
| | AATATGCTCAGCATGACGCAGCACTCCCTGAACTTTCCGGTTACATCACCCAATAGCTGAGATCAGA |
| 274 | > CRM_SP0112 |
| | CACTGAACCCTTGACCCCTGCCCTGCAGCCCCCGCAGCTTGCTGTTTGCCCACTCTATTTGCCCAGCCCCAGAGGTTAATTTTTAAAAAGCAGTCAAAAGTCCAAGTGGCCCTTGGCAGCATTTACTCTCTCTGTTTGCTCTGGT |
| | TAATAATCTCAGGAGCACAAACATTCCTTTGGAGAAGACAGAGCCAATGAGGCCCTCGTTCCAGGGAAACAGAATATGCTCAGCATGACGCAGCACTCCCTGAACTTTCCGGTTACATCACCCAATAGCTGAGATCAGA |
| 275 | > CRM_SP0113 |
| | CACTGAACCCTTGACCCCTGCCCTGCAGCCCCCGCAGCTTGCTGTTTGCCCACTCTATTTGCCCAGCCCCAGCCCTGGAGAGTCCTTTAGCAGGGCAAAGTGCAACATAGGCAGACCTTAAGGGATGACTCAGTAACAGATAAGC |
| | TTTGTGTGCCTGCAGGGGGATGGGAAGAGGGTGGGGCAGGAGAGGGACATAAAAGGGCTCTGAGGCATTGTACTGTGAATTCCTTCAGTCTCCTG |
| 276 | > CRM_SP0115 |
| | CCCTGGAGAGTCCTTTAGCAGGGCAAAGTGCAACATAGGCAGACCTTAAGGGATGACTCAGTAACAGATAAGCTTTGTGTGCCTGCAGGGGGATGGGAAGAGGGTGGGGCAGGAGAGGGACATAAAAGGGCTCTGAGGCATTGTA |
| | CTGTGAATTCCTTCAGTCTCCTGCTCTGCTCAGCCAGTCAGCCCTGCCTCCCTTGTTTAGGACCACACAGCACTGCTGGGTGTCTGCCTTTCCTTG |
| 277 | > CRM_SP0116 |
| | CCCTGGAGAGTCCTTTAGCAGGGCAAAGTGCAACATAGGCAGACCTTAAGGGATGACTCAGTAACAGATAAGCTTTGTGTGCCTGCAGGGGGATGGGAAGAGGGTGGGGCAGGAGAGGGACATAAAAGGGCTCTGAGGCATTGTA |
| | CTGTGAATTCCTTCAGTCTCCTG |
| 278 | > CRM_SP0121 |
| | CCCTGGAGAGTCCTTTAGCAGGGCAAAGTGCAACATAGGCAGACCTTAAGGGATGACTCAGTAACAGATAAGCTTTGTGTGCCTGCACCCTGGAGAGTCCTTTAGCAGGGCAAAGTGCAACATAGGCAGACCTTAAGGGATGACT |
| | CAGTAACAGATAAGCTTTGTGTGCCTGCA |
| 279 | > CRM_SP0124 |
| | CACTGAACCCTTGACCCCTGCCCTGCAGCCCCCGCAGCTTGCTGTTTGCCCACTCTATTTGCCCAGCCCCAGCCCTGGAGAGTCCTTTAGCAGGGCAAAGTGCAACATAGGCAGACCTTAAGGGATGACTCAGTAACAGATAAGC |
| | TTTGTGTGCCTGCA |
| 280 | > CRM_SP0127 (CRM_LVR_127) |
| | AGAATGAACATTGAACTTTGGACTATACCTGAGGGGTGAGGTAAACAACAGGACTATAAATGGCCCGGGAGGCGCCCTTTGGACCTTTTGCAATCCTGGCGCACTGAACCCTTGACCCCTGCCCTGCAGCCCCCGCAGCTTGCTG |
| | TTTGCCCACTCTATTTGCCCAGCCCCAGGGACAGAGCTGATCCTTGAACTCTTAAGTTCCAC |
| 281 | > CRM_SP0127A1 (CRM_LVR_127_A1) |
| | AGGTTAATTTTTAAAAAGCAGTCAAAAGTCCAAGTGGCCCTTGGCAGCATTTACTCTCTCTGTTTGCTCTGGTTAATAATCTCAGGAGCACAAACATTCCTGTACCAGAATGAACATTGAACTTTGGACTATACCTGAGGGGTGA |
| | GGTAAACAACAGGACTATAAATGGCCCGGGAGGCGCCCTTTGGACCTTTTGCAATCCTGGCGCACTGAACCCTTGACCCCTGCCCTGCAGCCCCCGCAGCTTGCTGTTTGCCCACTCTATTTGCCCAGCCCCAGGGACAGAGCTG |
| | ATCCTTGAACTCTTAAGTTCCAC |
| 282 | > CRM_SP0127V1 (CRM_LVR_127_V1) |
| | AAGCAAATATTTGTGGTTATGGATTAACTCGAACTGTTTGCCCACTCTATTTGCCCTGTACCAGAATGAACATTGAACTTTGGACTATACCTGAGGGGTGAGGTAAACAACAGGACTATAAATGGCCCGGGAGGCGCCCTTTGGA |
| | CCTTTTGCAATCCTGGCGCACTGAACCCTTGACCCCTGCCCTGCAGCCCCCGCAGCTTGCTGTTTGCCCACTCTATTTGCCCAGCCCCAGGGACAGAGCTGATCCTTGAACTCTTAAGTTCCAC |
| 283 | > CRM_SP0127V2 (CRM_LVR_127_V2) |
| | GGCGCCCTTTGGACCTTTTGCAATCCTGGAGCAAACAGCAAACACTGTACCAGAATGAACATTGAACTTTGGACTATACCTGAGGGGTGAGGTAAACAACAGGACTATAAATGGCCCGGGAGGCGCCCTTTGGACCTTTTGCAAT |
| | CCTGGCGCACTGAACCCTTGACCCCTGCCCTGCAGCCCCCGCAGCTTGCTGTTTGCCCACTCTATTTGCCCAGCCCCAGGGACAGAGCTGATCCTTGAACTCTTAAGTTCCAC |
| 284 | > CRM_SP0128 |
| | AGAATGAACATTGAACTTTGGACTATACCTGAGGGGTGAGGTAAACAACAGGACTATAAATAGAATGAACATTGAACTTTGGACTATACCTGAGGGGTGAGGTAAACAACAGGACTATAAATCACTGAACCCTTGACCCCTGCCC |
| | TGCAGCCCCCGCAGCTTGCTGTTTGCCCACTCTATTTGCCCAGCCCCAG |
| 285 | > CRM_SP0131 (CRM_LVR 131) |
| | GGCCCGGGAGGCGCCCTTTGGACCTTTTGCAATCCTGGCGCACTGAACCCTTGACCCCTGCCCTGCAGCCCCCGCAGCTTGCTGTTTGCCCACTCTATTTGCCCAGCCCCAGCCCTGGAGAGTCCTTTAGCAGGGCAAAGTGCAA |
| | CATAGGCAGACCTTAAGGGATGACTCAGTAACAGATAAGCTTTGTGTGCCTGCA |
| 286 | > CRM_SP0132 (CRM_LVR 132 |
| | AGAATGAACATTGAACTTTGGACTATACCTGAGGGGTGAGGTAAACAACAGGACTATAAATGGCCCGGGAGGCGCCCTTTGGACCTTTTGCAATCCTGGCGCACTGAACCCTTGACCCCTGCCCTGCAGCCCCCGCAGCTTGCTG |
| | TTTGCCCACTCTATTTGCCCAGCCCCAGCCCTGGAGAGTCCTTTAGCAGGGCAAAGTGCAACATAGGCAGACCTTAAGGGATGACTCAGTAACAGATAAGCTTTGTGTGCCTGCA |
| 287 | >CRM_SP0133 (CRM_LVR_133) |
| | AGAATGAACATTGAACTTTGGACTATACCTGAGGGGTGAGGTAAACAACAGGACTATAAATGGCCCGGGAGGCGCCCTTTGGACCTTTTGCAATCCTGGCG |
| 288 | > CRM_SP0155 |
| | GCTGGTTTCTTATAAAACTGATGGAAGATACAAACACTATTAAAGAACTGTTTGCATGTTGCAAATGATGTCCAAAGTCCAAACATTGTTAATAATTAATACTCCAATAAACATCATGTCAGAATTTCTGTTTTCTTTTCCCTTT |
| | GAACCTTTGCAGGATTGCCACATCATCAGGACCACACCTTCATCAGGAATGAATAT |
| 289 | > CRM_SP0158 |
| | ATTGGCATCTTCTATTGCTTTTCCTGGTGACTTCATTTTTCACTCTTGGCTAAAAATGGGTCTCTGATGATTTATTCTATCCTGGGTGTTGACAAGCTGAAGAAGTTGTGTGGGGCCTGCTGCCAGTAACCCTGGGTGACGAAGC |
| | GTGACTCACCACTCCGAGGTCAGTGGGGGGATGGAAGGCAGGGGAGTCAGCTGACAAGATCTGCTGCTTTGTCACCAGGCCTTCTGC |
| 290 | > CRM_SP0163 |
| | TGAAATGCCTGCCATATATTAGTGCCCTGAAGTCCAAAGGTAGAGGAACCGAGTGTTTAAAAATTACTGTGGCTGTGGAGTCAACATGATGTAAAAAAACAAACATTTGGATAACACCAAGAAGCCAGATATGGTTGAAATGTTG |
| | ACTGGTTGACAAAAATAATTTGGGTTGCTTAATGGTGCACAAAGGTAATGCAAAA |
| 291 | > CRM_SP0236 |
| | CAGGCTTTCACTTTCTCGCCAACTTACAAGGCCTTTCTGTGTAAACAATACCTGAACCTTTACCCCGTTGCCCGGCAACGGCCAGGTCTGTGCCAAGTGTTTG |
| 292 | > CRM_SP0239 |
| | CAGGCTTTCACTTTCTCGCCAACTTACAAGGCCTTTCTGTGTAAACAATACCTGAACCTTTACCCCGTTGCCCGGCAACGGCCAGGTCTGTGCCAAGTGTTTGAGGTTAATTTTTAAAAAGCAGTCAAAAGTCCAAGTGGCCCTT |
| | GGCAGCATTTACTCTCTCTGTTTGCTCTGGTTAATAATCTCAGGAGCACAAACATTCCGGCCCGGGAGGCGCCCTTTGGACCTTTTGCAATCCTGGCGCACTGAACCCTTGACCCCTGCCCTGCAGCCCCCGCAGCTTGCTGTTT |
| | GCCCACTCTATTTGCCCAGCCCCAGCCCTGGAGAGTCCTTTAGCAGGGCAAAGTGCAACATAGGCAGACCTTAAGGGATGACTCAGTAACAGATAAGCTTTGTGTGCCTGCA |
| 293 | > CRM_SP0240 |
| | CAGGCTTTCACTTTCTCGCCAACTTACAAGGCCTTTCTGTGTAAACAATACCTGAACCTTTACCCCGTTGCCCGGCAACGGCCAGGTCTGTGCCAAGTGTTTG |
| 294 | > CRM_SP0241 |
| | AGGTTAATTTTTAAAAAGCAGTCAAAAGTCCAAGTGGCCCTTGGCAGCATTTACTCTCTCTGTTTGCTCTGGTTAATAATCTCAGGAGCACAAACATTCCGGCCCGGGAGGCGCCCTTTGGACCTTTTGCAATCCTGGCGCACTG |
| | AACCCTTGACCCCTGCCCTGCAGCCCCCGCAGCTTGCTGTTTGCCCACTCTATTTGCCCAGCCCCAGCCCTGGAGAGTCCTTTAGCAGGGCAAAGTGCAACATAGGCAGACCTTAAGGGATGACTCAGTAACAGATAAGCTTTGT |
| | GTGCCTGCA |
| 295 | > CRM_SP0242 |
| | AGGTTAATTTTTAAAAAGCAGTCAAAAGTCCAAGTGGCCCTTGGCAGCATTTACTCTCTCTGTTTGCTCTGGTTAATAATCTCAGGAGCACAAACATTCCGGCCCGGGAGGCGCCCTTTGGACCTTTTGCAATCCTGGCGCACTG |
| | AACCCTTGACCCCTGCCCTGCAGCCCCCGCAGCTTGCTGTTTGCCCACTCTATTTGCCCAGCCCCAG |
| 296 | > CRM_SP0243 |
| | TGAAATGCCTGCCATATATTAGTGCCCTGAAGTCCAAAGGTAGAGGAACCGAGTGTTTAAAAATTACTGTGGCTGTGGAGTCAACATGATGTAAAAAAACAAACATTTGGATAACACCAAGAAGCCAGATATGGTTGAAATGTTG |
| | ACTGGTTGACAAAAATAATTTGGGTTGCTTAATGGTGCACAAAGGTAATGCAAAAAGGTTAATTTTTAAAAAGCAGTCAAAAGTCCAAGTGGCCCTTGGCAGCATTTACTCTCTCTGTTTGCTCTGGTTAATAATCTCAGGAGCA |
| | CAAACATTCCGGCCCGGGAGGCGCCCTTTGGACCTTTTGCAATCCTGGCGCACTGAACCCTTGACCCCTGCCCTGCAGCCCCCGCAGCTTGCTGTTTGCCCACTCTATTTGCCCAGCCCCAGCCCTGGAGAGTCCTTTAGCAGGG |
| | CAAAGTGCAACATAGGCAGACCTTAAGGGATGACTCAGTAACAGATAAGCTTTGTGTGCCTGCA |
| 297 | > CRM_SP0244 |
| | CTTTGGACCTTTTGCAATCCTGGCGCACTGAACCCTTGACCCCTGCCCTGCAGCCCCCGCAGCTTGCTGTTTGCCCACTCTATTTGCCCAGCCCCAGTGAAATGCCTGCCATATATTAGTGCCCTGAAGTCCAAAGGTAGAGGAA |
| | CCGAGTGTTTAAAAATTACTGTGGCTGTGGAGTCAACATGATGTAAAAAAACAAACATTTGGATAACACCAAGAAGCCAGATATGGTTGAAATGTTGACTGGTTGACAAAAATAATTTGGGTTGCTTAATGGTGCACAAAGGTAA |
| | TGCAAAA |
| 298 | > CRM_SP0246 |
| | AGGTTAATTTTTAAAAAGCAGTCAAAAGTCCAAGTGGCCCTTGGCAGCATTTACTCTCTCTGTTTGCTCTGGTTAATAATCTCAGGAGCACAAACATTCCGGCCCGGGAGGCGCCCTTTGGACCTTTTGCAATCCTGGCGCACTG |
| | AACCCTTGACCCCTGCCCTGCAGCCCCCGCAGCTTGCTGTTTGCCCACTCTATTTGCCCAGCCCCAG |
| 299 | > CRM_SP0247 |
| | GCTGGTTTCTTATAAAACTGATGGAAGATACAAACACTATTAAAGAACTGTTTGCATGTTGCAAATGATGTCCAAAGTCCAAACATTGTTAATAATTAATACTCCAATAAACATCATGTCAGAATTTCTGTTTTCTTTTCCCTTT |
| | GAACCTTTGCAGGATTGCCACATCATCAGGACCACACCTTCATCAGGAATGAATATCAGGCTTTCACTTTCTCGCCAACTTACAAGGCCTTTCTGTGTAAACAATACCTGAACCTTTACCCCGTTGCCCGGCAACGGCCAGGTCT |
| | GTGCCAAGTGTTTG |
| 300 | > CRM_SP0248 |
| | AGGTTAATTTTTAAAAAGCAGTCAAAAGTCCAAGTGGCCCTTGGCAGCATTTACTCTCTCTGTTTGCTCTGGTTAATAATCTCAGGAGCACAAACATTCCGGCCCGGGAGGCGCCCTTTGGACCTTTTGCAATCCTGGCGCACTG |
| | AACCCTTGACCCCTGCCCTGCAGCCCCCGCAGCTTGCTGTTTGCCCACTCTATTTGCCCAGCCCCAGCAGGCTTTCACTTTCTCGCCAACTTACAAGGCCTTTCTGTGTAAACAATACCTGAACCTTTACCCCGTTGCCCGGCAA |
| | CGGCCAGGTCTGTGCCAAGTGTTTG |
| 301 | > CRM_SP0249 |
| | AGGTTAATTTTTAAAAAGCAGTCAAAAGTCCAAGTGGCCCTTGGCAGCATTTACTCTCTCTGTTTGCTCTGGTTAATAATCTCAGGAGCACAAACATTCCGGCCCGGGAGGCGCCCTTTGGACCTTTTGCAATCCTGGCGCACTG |
| | AACCCTTGACCCCTGCCCTGCAGCCCCCGCAGCTTGCTGTTTGCCCACTCTATTTGCCCAGCCCCAGCAGGCTTTCACTTTCTCGCCAACTTACAAGGCCTTTCTGTGTAAACAATACCTGAACCTTTACCCCGTTGCCCGGCAA |
| | CGGCCAGGTCTGTGCCAAGTGTTTG |
| 302 | > CRM_SP0250 |
| | AGGTTAATTTTTAAAAAGCAGTCAAAAGTCCAAGTGGCCCTTGGCAGCATTTACTCTCTCTGTTTGCTCTGGTTAATAATCTCAGGAGCACAAACATTCCGGCCCGGGAGGCGCCCTTTGGACCTTTTGCAATCCTGGCGCACTG |
| | AACCCTTGACCCCTGCCCTGCAGCCCCCGCAGCTTGCTGTTTGCCCACTCTATTTGCCCAGCCCCAGGCTGGTTTCTTATAAAACTGATGGAAGATACAAACACTATTAAAGAACTGTTTGCATGTTGCAAATGATGTCCAAAGT |
| | CCAAACATTGTTAATAATTAATACTCCAATAAACATCATGTCAGAATTTCTGTTTTCTTTTCCCTTTGAACCTTTGCAGGATTGCCACATCATCAGGACCACACCTTCATCAGGAATGAATAT |
| 303 | > CRM_SP0251 |
| | AGGTTAATTTTTAAAAAGCAGTCAAAAGTCCAAGTGGCCCTTGGCAGCATTTACTCTCTCTGTTTGCTCTGGTTAATAATCTCAGGAGCACAAACATTCCGGCCCGGGAGGCGCCCTTTGGACCTTTTGCAATCCTGGCGCACTG |
| | AACCCTTGACCCCTGCCCTGCAGCCCCCGCAGCTTGCTGTTTGCCCACTCTATTTGCCCAGCCCCAGAAGCAAATATTTGTGGTTATGGATTAACTCGAACTGTTTGCCCACTCTATTTGCCC |
| 304 | > CRM_SP0252 |
| | AAGCAAATATTTGTGGTTATGGATTAACTCGAACTGTTTGCCCACTCTATTTGCCCCAGGCTTTCACTTTCTCGCCAACTTACAAGGCCTTTCTGTGTAAACAATACCTGAACCTTTACCCCGTTGCCCGGCAACGGCCAGGTCT |
| | GTGCCAAGTGTTTG |
| 305 | > CRM_SP0253 |
| | AAGCAAATATTTGTGGTTATGGATTAACTCGAACTGTTTGCCCACTCTATTTGCCCCAGGCTTTCACTTTCTCGCCAACTTACAAGGCCTTTCTGTGTAAACAATACCTGAACCTTTACCCCGTTGCCCGGCAACGGCCAGGTCT |
| | GTGCCAAGTGTTTG |
| 306 | > CRM_SP0254 |
| | AGGTTAATTTTTAAAAAGCAGTCAAAAGTCCAAGTGGCCCTTGGCAGCATTTACTCTCTCTGTTTGCTCTGGTTAATAATCTCAGGAGCACAAACATTCCGGCCCGGGAGGCGCCCTTTGGACCTTTTGCAATCCTGGCGCAGGC |
| | TTTCACTTTCTCGCCAACTTACAAGGCCTTTCTGTGTAAACAATACCTGAACCTTTACCCCGTTGCCCGGCAACGGCCAGGTCTGTGCCAAGTGTTTG |
| 307 | > CRM_SP0255 |
| | AGGTTAATTTTTAAAAAGCAGTCAAAAGTCCAAGTGGCCCTTGGCAGCATTTACTCTCTCTGTTTGCTCTGGTTAATAATCTCAGGAGCACAAACATTCCGGCCCGGGAGGCGCCCTTTGGACCTTTTGCAATCCTGGCGCAGGC |
| | TTTCACTTTCTCGCCAACTTACAAGGCCTTTCTGTGTAAACAATACCTGAACCTTTACCCCGTTGCCCGGCAACGGCCAGGTCTGTGCCAAGTGTTTG |
| 308 | > CRM_SP0256 |
| | ATTGGCATCTTCTATTGCTTTTCCTGGTGACTTCATTTTTCACTCTTGGCTAAAAATGGGTCTCTGATGATTTATTCTATCCTGGGTGTTGACAAGCTGAAGAAGTTGTGTGGGGCCTGCTGCCAGTAACCCTGGGTGACGAAGC |
| | GTGACTCACCACTCCGAGGTCAGTGGGGGGATGGAAGGCAGGGGAGTCAGCTGACAAGATCTGCTGCTTTGTCACCAGGCCTTCTGCCAGGCTTTCACTTTCTCGCCAACTTACAAGGCCTTTCTGTGTAAACAATACCTGAACC |
| | TTTACCCCGTTGCCCGGCAACGGCCAGGTCTGTGCCAAGTGTTTG |
| 309 | > CRM_SP0257 |
| | CAGGCTTTCACTTTCTCGCCAACTTACAAGGCCTTTCTGTGTAAACAATACCTGAACCTTTACCCCGTTGCCCGGCAACGGCCAGGTCTGTGCCAAGTGTTTGGCTGGTTTCTTATAAAACTGATGGAAGATACAAACACTATTA |
| | AAGAACTGTTTGCATGTTGCAAATGATGTCCAAAGTCCAAACATTGTTAATAATTAATACTCCAATAAACATCATGTCAGAATTTCTGTTTTCTTTTCCCTTTGAACCTTTGCAGGATTGCCACATCATCAGGACCACACCTTCA |
| | TCAGGAATGAATAT |
| 310 | > CRM_SP0258 |
| | CTGTTTGCTGCTTGCAATGTTTGCCCATTTTAGGGAGGTTAATTTTTAAAAAGCAGTCAAAAGTCCAAGTGGCCCTTGGCAGCATTTACTCTCTCTGTTTGCTCTGGTTAATAATCTCAGGAGCACAAACATTCCGGCCCGGGAG |
| | GCGCCCTTTGGACCTTTTGCAATCCTGGCGCACTGAACCCTTGACCCCTGCCCTGCAGCCCCCGCAGCTTGCTGTTTGCCCACTCTATTTGCCCAGCCCCAGCCCTGGAGAGTCCTTTAGCAGGGCAAAGTGCAACATAGGCAGA |
| | CCTTAAGGGATGACTCAGTAACAGATAAGCTTTGTGTGCCTGCA |
| 311 | > CRM_SP0259 |
| | CTGTTTGCTGCTTGCAATGTTTGCCCATTTTAGGGGCTGGTTTCTTATAAAACTGATGGAAGATACAAACACTATTAAAGAACTGTTTGCATGTTGCAAATGATGTCCAAAGTCCAAACATTGTTAATAATTAATACTCCAATAA |
| | ACATCATGTCAGAATTTCTGTTTTCTTTTCCCTTTGAACCTTTGCAGGATTGCCACATCATCAGGACCACACCTTCATCAGGAATGAATAT |
| 312 | > CRM_SP0264 |
| | CAGGCTTTCACTTTCTCGCCAACTTACAAGGCCTTTCTGTGTAAACAATACCTGAACCTTTACCCCGTTGCCCGGCAACGGCCAGGTCTGTGCCAAGTGTTTG |
| 313 | > CRM_SP0265 (CRM_LVR_131_A1) |
| | AGGTTAATTTTTAAAAAGCAGTCAAAAGTCCAAGTGGCCCTTGGCAGCATTTACTCTCTCTGTTTGCTCTGGTTAATAATCTCAGGAGCACAAACATTCCTGTACCGGCCCGGGAGGCGCCCTTTGGACCTTTTGCAATCCTGGC |
| | GCACTGAACCCTTGACCCCTGCCCTGCAGCCCCCGCAGCTTGCTGTTTGCCCACTCTATTTGCCCAGCCCCAGCCCTGGAGAGTCCTTTAGCAGGGCAAAGTGCAACATAGGCAGACCTTAAGGGATGACTCAGTAACAGATAAG |
| | CTTTGTGTGCCTGCA |
| 314 | > CRM_SP0266 (CRM_LVR 131_V1) |
| | AAGCAAATATTTGTGGTTATGGATTAACTCGAACTGTTTGCCCACTCTATTTGCCCTGTACCGGCCCGGGAGGCGCCCTTTGGACCTTTTGCAATCCTGGCGCACTGAACCCTTGACCCCTGCCCTGCAGCCCCCGCAGCTTGCT |
| | GTTTGCCCACTCTATTTGCCCAGCCCCAGCCCTGGAGAGTCCTTTAGCAGGGCAAAGTGCAACATAGGCAGACCTTAAGGGATGACTCAGTAACAGATAAGCTTTGTGTGCCTGCA |
| 315 | > CRM_SP0267 (CRM_LVR 131_V2) |
| | GGCGCCCTTTGGACCTTTTGCAATCCTGGAGCAAACAGCAAACACTGTACCGGCCCGGGAGGCGCCCTTTGGACCTTTTGCAATCCTGGCGCACTGAACCCTTGACCCCTGCCCTGCAGCCCCCGCAGCTTGCTGTTTGCCCACT |
| | CTATTTGCCCAGCCCCAGCCCTGGAGAGTCCTTTAGCAGGGCAAAGTGCAACATAGGCAGACCTTAAGGGATGACTCAGTAACAGATAAGCTTTGTGTGCCTGCA |
| 316 | > CRM_SP0268 (CRM_LVR 132_A1) |
| | AGGTTAATTTTTAAAAAGCAGTCAAAAGTCCAAGTGGCCCTTGGCAGCATTTACTCTCTCTGTTTGCTCTGGTTAATAATCTCAGGAGCACAAACATTCCTGTACCAGAATGAACATTGAACTTTGGACTATACCTGAGGGGTGA |
| | GGTAAACAACAGGACTATAAATGGCCCGGGAGGCGCCCTTTGGACCTTTTGCAATCCTGGCGCACTGAACCCTTGACCCCTGCCCTGCAGCCCCCGCAGCTTGCTGTTTGCCCACTCTATTTGCCCAGCCCCAGCCCTGGAGAGT |
| | CCTTTAGCAGGGCAAAGTGCAACATAGGCAGACCTTAAGGGATGACTCAGTAACAGATAAGCTTTGTGTGCCTGCA |
| 317 | > CRM_SP0269 (CRM_LVR_132_V1) |
| | AAGCAAATATTTGTGGTTATGGATTAACTCGAACTGTTTGCCCACTCTATTTGCCCTGTACCAGAATGAACATTGAACTTTGGACTATACCTGAGGGGTGAGGTAAACAACAGGACTATAAATGGCCCGGGAGGCGCCCTTTGGA |
| | CCTTTTGCAATCCTGGCGCACTGAACCCTTGACCCCTGCCCTGCAGCCCCCGCAGCTTGCTGTTTGCCCACTCTATTTGCCCAGCCCCAGCCCTGGAGAGTCCTTTAGCAGGGCAAAGTGCAACATAGGCAGACCTTAAGGGATG |
| | ACTCAGTAACAGATAAGCTTTGTGTGCCTGCA |
| 318 | > CRM_SP0270 (CRM_LVR_132_V2) |
| | GGCGCCCTTTGGACCTTTTGCAATCCTGGAGCAAACAGCAAACACTGTACCAGAATGAACATTGAACTTTGGACTATACCTGAGGGGTGAGGTAAACAACAGGACTATAAATGGCCCGGGAGGCGCCCTTTGGACCTTTTGCAAT |
| | CCTGGCGCACTGAACCCTTGACCCCTGCCCTGCAGCCCCCGCAGCTTGCTGTTTGCCCACTCTATTTGCCCAGCCCCAGCCCTGGAGAGTCCTTTAGCAGGGCAAAGTGCAACATAGGCAGACCTTAAGGGATGACTCAGTAACA |
| | GATAAGCTTTGTGTGCCTGCA |
| 319 | > CRM_SP0271 (CRM_LVR_133_A1) |
| | AGGTTAATTTTTAAAAAGCAGTCAAAAGTCCAAGTGGCCCTTGGCAGCATTTACTCTCTCTGTTTGCTCTGGTTAATAATCTCAGGAGCACAAACATTCCTGTACCAGAATGAACATTGAACTTTGGACTATACCTGAGGGGTGA |
| | GGTAAACAACAGGACTATAAATGGCCCGGGAGGCGCCCTTTGGACCTTTTGCAATCCTGGCG |
| 320 | > CRM_SP0272 (CRM_LVR 133_V1) |
| | AAGCAAATATTTGTGGTTATGGATTAACTCGAACTGTTTGCCCACTCTATTTGCCCTGTACCAGAATGAACATTGAACTTTGGACTATACCTGAGGGGTGAGGTAAACAACAGGACTATAAATGGCCCGGGAGGCGCCCTTTGGA |
| | CCTTTTGCAATCCTGGCG |
| 321 | > CRM_SP0273 (CRM_LVR 133 V2) |
| | GGCGCCCTTTGGACCTTTTGCAATCCTGGAGCAAACAGCAAACACTGTACCAGAATGAACATTGAACTTTGGACTATACCTGAGGGGTGAGGTAAACAACAGGACTATAAATGGCCCGGGAGGCGCCCTTTGGACCTTTTGCAAT |
| | CCTGGCG |
| 322 | > CRM_SP0368 |
| | AGGTTAATTTTTAAAAAGCAGTCAAAAGTCCAAGTGGCCCTTGGCAGCATTTACTCTCTCTGTTTGCTCTGGTTAATAATCTCAGGAGCACAAACATTCC |
| 323 | > CRM_SP0373 |
| | AGGTTAATTTTTAAAAAGCAGTCAAAAGTCCAAGTGGCCCTTGGCAGCATTTACTCTCTCTGTTTGCTCTGGTTAATAATCTCAGGAGCACAAACATTCCTGTACCGGCCCGGGAGGCGCCCTTTGGACCTTTTGCAATCCTGGC |
| | G |
| 324 | > CRM_SP0378 |
| | TGCTCTCTGACAAAGATACGGTGGGTCCCACTGATGAACTGTGCTGCCACAGTAAATGTAGCCACTATGCCTATCTCCATTCTGAAGATGTGCCCTGTTCAAACATGTCCTAATACTCTGTCTCTGCAAGGGTCATCAGTAGTTT |
| | TCCATCTTACTCAACATCCTCCCAGTG |
| 325 | > CRM_SP0379 |
| | CCTCCCCGTGTTCCTGCTCTTTGTCCCTCTGTCCTACTTAGACTAATATTTGCCTTGGGTACTGCAAACAGGAAATGGGGGAGGGACAGGAGTAGGGCGGCCCTGTTCAAACATGTCCTAATACTCTGTCTCTGCAAGGGTCATC |
| | AGTAGTTTTCCATCTTACTCAACATCCTCCCAGTG |
| 326 | > CRM_SP0380 |
| | CCGCCCCCACTGAACCCTTGACCCCTGCCCTGCAGCCCCCGCAGCTTGCTGTTTGCCCACTCTATTTGCCCAGCCCCAGCCCTGTTCAAACATGTCCTAATACTCTGTCTCTGCAAGGGTCATCAGTAGTTTTCCATCTTACTCA |
| | ACATCCTCCCAGTG |
| 327 | > CRM_SP0381 |
| | AAGCTTTCTGAACAGCCAAACAGAGATTCCAAAGTTCAGGCACCAAAGTTCAGACCCTAACAGTTATTTACAAGGGTCAGTTAACCCCTGTTCAAACATGTCCTAATACTCTGTCTCTGCAAGGGTCATCAGTAGTTTTCCATCT |
| | TACTCAACATCCTCCCAGTG |
| 328 | > CRM_SP0384 |
| | AGGTTAATTTTTAAAAAGCAGTCAAAAGTCCAAGTGGCCCTTGGCAGCATTTACTCTCTCTGTTTGCTCTGGTTAATAATCTCAGGAGCACAAACATTCCGGCCCGGGAGGCGCCCTTTGGACCTTTTGCAATCCTGGCG |
| 329 | > CRM_SP0388 |
| | AGGTTAATTTTTAAAAAGCAGTCAAAAGTCCAAGTGGCCCTTGGCAGCATTTACTCTCTCTGTTTGCTCTGGTTAATAATCTCAGGAGCACAAACATTCC |
| 330 | > CRM_SP0396 |
| | AGGTTAATTTTTAAAAAGCAGTCAAAAGTCCAAGTGGCCCTTGGCAGCATTTACTCTCTCTGTTTGCTCTGGTTAATAATCTCAGGAGCACAAACATTCCTGTACCGGCCCGGGAGGCGCCCTTTGGACCTTTTGCAATCCTGGC |
| | GCCCTGGAGAGTCCTTTAGCAGGGCAAAGTGCAACATAGGCAGACCTTAAGGGATGACTCAGTAACAGATAAGCTTTGTGTGCCTGCA |
| 331 | > CRM_SP0397 |
| | AGGTTAATTTTTAAAAAGCAGTCAAAAGTCCAAGTGGCCCTTGGCAGCATTTACTCTCTCTGTTTGCTCTGGTTAATAATCTCAGGAGCACAAACATTCCTGTACCCACTGAACCCTTGACCCCTGCCCTGCAGCCCCCGCAGCT |
| | TGCTGTTTGCCCACTCTATTTGCCCAGCCCCAGCCCTGGAGAGTCCTTTAGCAGGGCAAAGTGCAACATAGGCAGACCTTAAGGGATGACTCAGTAACAGATAAGCTTTGTGTGCCTGCA |
| 332 | > CRM_SP0398 |
| | CAGGCTTTCACTTTCTCGCCAACTTACAAGGCCTTTCTGTGTAAACAATACCTGAACCTTTACCCCGTTGCCCGGCAACGGCCAGGTCTGTGCCAAGTGTTTGAGGTTAATTTTTAAAAAGCAGTCAAAAGTCCAAGTGGCCCTT |
| | GGCAGCATTTACTCTCTCTGTTTGCTCTGGTTAATAATCTCAGGAGCACAAACATTCCTGTACCAGAATGAACATTGAACTTTGGACTATACCTGAGGGGTGAGGTAAACAACAGGACTATAAATGGCCCGGGAGGCGCCCTTTG |
| | GACCTTTTGCAATCCTGGCGCACTGAACCCTTGACCCCTGCCCTGCAGCCCCCGCAGCTTGCTGTTTGCCCACTCTATTTGCCCAGCCCCAGCCCTGGAGAGTCCTTTAGCAGGGCAAAGTGCAACATAGGCAGACCTTAAGGGA |
| | TGACTCAGTAACAGATAAGCTTTGTGTGCCTGCA |
| 333 | > CRM_SP0399 |
| | AGAATGAACATTGAACTTTGGACTATACCTGAGGGGTGAGGTAAACAACAGGACTATAAAT |
| 334 | > CRM_SP0403 |
| | CAGGCTTTCACTTTCTCGCCAACTTACAAGGCCTTTCTGTGTAAACAATACCTGAACCTTTACCCCGTTGCCCGGCAACGGCCAGGTCTGTGCCAAGTGTTTGAAGCAAATATTTGTGGTTATGGATTAACTCGAACTGTTTGCC |
| | CACTCTATTTGCCCTGTACCGGCCCGGGAGGCGCCCTTTGGACCTTTTGCAATCCTGGCGCACTGAACCCTTGACCCCTGCCCTGCAGCCCCCGCAGCTTGCTGTTTGCCCACTCTATTTGCCCAGCCCCAGCCCTGGAGAGTCC |
| | TTTAGCAGGGCAAAGTGCAACATAGGCAGACCTTAAGGGATGACTCAGTAACAGATAAGCTTTGTGTGCCTGCA |
| 335 | > CRM_SP0404 |
| | CAGGCTTTCACTTTCTCGCCAACTTACAAGGCCTTTCTGTGTAAACAATACCTGAACCTTTACCCCGTTGCCCGGCAACGGCCAGGTCTGTGCCAAGTGTTTGAAGCAAATATTTGTGGTTATGGATTAACTCGAACTGTTTGCC |
| | CACTCTATTTGCCCTGTACCAGAATGAACATTGAACTTTGGACTATACCTGAGGGGTGAGGTAAACAACAGGACTATAAATGGCCCGGGAGGCGCCCTTTGGACCTTTTGCAATCCTGGCGCACTGAACCCTTGACCCCTGCCCT |
| | GCAGCCCCCGCAGCTTGCTGTTTGCCCACTCTATTTGCCCAGCCCCAGCCCTGGAGAGTCCTTTAGCAGGGCAAAGTGCAACATAGGCAGACCTTAAGGGATGACTCAGTAACAGATAAGCTTTGTGTGCCTGCA |
| 336 | > CRM_SP0405 |
| | CAGGCTTTCACTTTCTCGCCAACTTACAAGGCCTTTCTGTGTAAACAATACCTGAACCTTTACCCCGTTGCCCGGCAACGGCCAGGTCTGTGCCAAGTGTTTGAAGCAAATATTTGTGGTTATGGATTAACTCGAACTGTTTGCC |
| | CACTCTATTTGCCCTGTACCAGAATGAACATTGAACTTTGGACTATACCTGAGGGGTGAGGTAAACAACAGGACTATAAATGGCCCGGGAGGCGCCCTTTGGACCTTTTGCAATCCTGGCGCACTGAACCCTTGACCCCTGCCCT |
| | GCAGCCCCCGCAGCTTGCTGTTTGCCCACTCTATTTGCCCAGCCCCAG |
| 337 | > CRM_SP0406 |
| | AGGTTAATTTTTAAAAAGCAGTCAAAAGTCCAAGTGGCCCTTGGCAGCATTTACTCTCTCTGTTTGCTCTGGTTAATAATCTCAGGAGCACAAACATTCCGGCCCGGGAGGCGCCCTTTGGACCTTTTGCAATCCTGGCG |
| 338 | > CRM_SP0407 |
| | AGGTTAATTTTTAAAAAGCAGTCAAAAGTCCAAGTGGCCCTTGGCAGCATTTACTCTCTCTGTTTGCTCTGGTTAATAATCTCAGGAGCACAAACATTCCCAGGCTTTCACTTTCTCGCCAACTTACAAGGCCTTTCTGTGTAAA |
| | CAATACCTGAACCTTTACCCCGTTGCCCGGCAACGGCCAGGTCTGTGCCAAGTGTTTG |
| 339 | > CRM_SP0409 |
| | AGGTTAATTTTTAAAAAGCAGTCAAAAGTCCAAGTGGCCCTTGGCAGCATTTACTCTCTCTGTTTGCTCTGGTTAATAATCTCAGGAGCACAAACATTCCCCCTGTTCAAACATGTCCTAATACTCTGTCTCTGCAAGGGTCATC |
| | AGTAGTTTTCCATCTTACTCAACATCCTCCCAGTG |
| 340 | > CRM_SP0411 |
| | CCCTGTTCAAACATGTCCTAATACTCTGTCTCTGCAAGGGTCATCAGTAGTTTTCCATCTTACTCAACATCCTCCCAGTG |
| 86 | > CRM_SP0412 |
| | AGGTTAATTTTTAAAAAGCAGTCAAAAGTCCAAGTGGCCCTTGGCAGCATTTACTCTCTCTGTTTGCTCTGGTTAATAATCTCAGGAGCACAAACATTCCCCCTGTTCAAACATGTCCTAATACTCTGTCTCTGCAAGGGTCATC |
| | AGTAGTTTTCCATCTTACTCAACATCCTCCCAGTG (SEQ ID NO: 186) |
| 341 | > CRM_SP0413 |
| | AGGTTAATTTTTAAAAAGCAGTCAAAAGTCCAAGTGGCCCTTGGCAGCATTTACTCTCTCTGTTTGCTCTGGTTAATAATCTCAGGAGCACAAACATTCCTGTACCGGCCCGGGAGGCGCCCTTTGGACCTTTTGCAATCCTGGC |
| | G |
| 342 | > SP0107 |
| | CCCTGGAGAGTCCTTTAGCAGGGCAAAGTGCAACATAGGCAGACCTTAAGGGATGACTCAGTAACAGATAAGCTTTGTGTGCCTGCAGGGGGATGGGAAGAGGGTGGGGCAGGAGAGGGACATAAAAGGGCTCTGAGGCATTGTA |
| | CTGTGAATTCCTTCAGTCTCCTGTTTGGAGAAGACAGAGCCAATGAGGCCCTCGTTCCAGGGAAACAGAATATGCTCAGCATGACGCAGCACTCCCTGAACTTTCCGGTTACATCACCCAATAGCTGAGATCAGAGGGCATATAA |
| | AACAGGGGCAAGGCACAGACTCATAGCAGAGCAATCACCACCAAGCCTGGAATAACTGCAGCCACC |
| 343 | > SP0109 |
| | CCTGGAGAGTCCTTTAGCAGGGCAAAGTGCAACATAGGCAGACCTTAAGGGATGACTCAGTAACAGATAAGCTTTGTGTGCCTGCAGGGGGATGGGAAGAGGGTGGGGCAGGAGAGGGACATAAAAGGGCTCTGAGGCATTGTAC |
| | TGTGAATTCCTTCAGTCTCCTGCTCTGCTCAGCCAGTCAGCCCTGCCTCCCTTGTTTAGGACCACACAGCACTGCTGGGTGTCTGCCTTTCCTTGGGTAATTTTTTTTTCTGGTTAATATTTAGCAAGAATTCTGCAGAGTGATC |
| | AAAAAAATCAAATACTCAGTATTTCAGAAATAGATTAAATAGGTTACTTTTTTACTGATAATGTGAAAGAATGATATAAAAACTTGATTTTCCTCAACAACATTACTTTCTTTTGTAAATGTGGTTTCTACAAAGATGAAACTAC |
| | TAAAACTTACAGGCCACC |
| 344 | > SP0111 |
| | AGGTTAATTTTTAAAAAGCAGTCAAAAGTCCAAGTGGCCCTTGGCAGCATTTACTCTCTCTGTTTGCTCTGGTTAATAATCTCAGGAGCACAAACATTCCTTTGGAGAAGACAGAGCCAATGAGGCCCTCGTTCCAGGGAAACAG |
| | AATATGCTCAGCATGACGCAGCACTCCCTGAACTTTCCGGTTACATCACCCAATAGCTGAGATCAGAGGGCATATAAAACAGGGGCAAGGCACAGACTCATAGCAGAGCAATCACCACCAAGCCTGGAATAACTGCAGCCACC |
| 345 | > SP0112 |
| | CACTGAACCCTTGACCCCTGCCCTGCAGCCCCCGCAGCTTGCTGTTTGCCCACTCTATTTGCCCAGCCCCAGAGGTTAATTTTTAAAAAGCAGTCAAAAGTCCAAGTGGCCCTTGGCAGCATTTACTCTCTCTGTTTGCTCTGGT |
| | TAATAATCTCAGGAGCACAAACATTCCTTTGGAGAAGACAGAGCCAATGAGGCCCTCGTTCCAGGGAAACAGAATATGCTCAGCATGACGCAGCACTCCCTGAACTTTCCGGTTACATCACCCAATAGCTGAGATCAGAGGGCAT |
| | ATAAAACAGGGGCAAGGCACAGACTCATAGCAGAGCAATCACCACCAAGCCTGGAATAACTGCAGCCACC |
| 346 | > SP0113 |
| | CACTGAACCCTTGACCCCTGCCCTGCAGCCCCCGCAGCTTGCTGTTTGCCCACTCTATTTGCCCAGCCCCAGCCCTGGAGAGTCCTTTAGCAGGGCAAAGTGCAACATAGGCAGACCTTAAGGGATGACTCAGTAACAGATAAGC |
| | TTTGTGTGCCTGCAGGGGGATGGGAAGAGGGTGGGGCAGGAGAGGGACATAAAAGGGCTCTGAGGCATTGTACTGTGAATTCCTTCAGTCTCCTGGGGCATATAAAACAGGGGCAAGGCACAGACTCATAGCAGAGCAATCACCA |
| | CCAAGCCTGGAATAACTGCAGCCACC |
| 347 | > SP0115 |
| | CCCTGGAGAGTCCTTTAGCAGGGCAAAGTGCAACATAGGCAGACCTTAAGGGATGACTCAGTAACAGATAAGCTTTGTGTGCCTGCAGGGGGATGGGAAGAGGGTGGGGCAGGAGAGGGACATAAAAGGGCTCTGAGGCATTGTA |
| | CTGTGAATTCCTTCAGTCTCCTGCTCTGCTCAGCCAGTCAGCCCTGCCTCCCTTGTTTAGGACCACACAGCACTGCTGGGTGTCTGCCTTTCCTTGTGGACTTAGCCCCTGTTTGCTCCTCCGATAACTGGGGTGACCTTGGTTA |
| | ATATTCACCAGCAGCCTCCCCCGTTGCCCCTCTGGATCCACTGCTTAAATACGGACGAGGACAGGGCCCTGTCTCCTCAGCTTCAGGCACCACCACTGACCTGGGACAGTGAATCGCCACC |
| 348 | > SP0116 |
| | CCCTGGAGAGTCCTTTAGCAGGGCAAAGTGCAACATAGGCAGACCTTAAGGGATGACTCAGTAACAGATAAGCTTTGTGTGCCTGCAGGGGGATGGGAAGAGGGTGGGGCAGGAGAGGGACATAAAAGGGCTCTGAGGCATTGTA |
| | CTGTGAATTCCTTCAGTCTCCTGTGGACTTAGCCCCTGTTTGCTCCTCCGATAACTGGGGTGACCTTGGTTAATATTCACCAGCAGCCTCCCCCGTTGCCCCTCTGGATCCACTGCTTAAATACGGACGAGGACAGGGCCCTGTC |
| | TCCTCAGCTTCAGGCACCACCACTGACCTGGGACAGTGAATCGCCACC |
| 349 | > SP0121 |
| | CCCTGGAGAGTCCTTTAGCAGGGCAAAGTGCAACATAGGCAGACCTTAAGGGATGACTCAGTAACAGATAAGCTTTGTGTGCCTGCACCCTGGAGAGTCCTTTAGCAGGGCAAAGTGCAACATAGGCAGACCTTAAGGGATGACT |
| | CAGTAACAGATAAGCTTTGTGTGCCTGCAGGGCATATAAAACAGGGGCAAGGCACAGACTCATAGCAGAGCAATCACCACCAAGCCTGGAATAACTGCAGCCACC |
| 350 | > SP0124 |
| | CACTGAACCCTTGACCCCTGCCCTGCAGCCCCCGCAGCTTGCTGTTTGCCCACTCTATTTGCCCAGCCCCAGCCCTGGAGAGTCCTTTAGCAGGGCAAAGTGCAACATAGGCAGACCTTAAGGGATGACTCAGTAACAGATAAGC |
| | TTTGTGTGCCTGCACTCTTTTGTTTTACATGAAGGGTCTGGCAGCCAAAGCAATCACTCAAAGTTCAAACCTTATCATTTTTTGCTTTGTTCCTCTTGGCCTTGGTTTTGTACATCAGCTTTGAAAATACCATCCCAGGGTTAAT |
| | GCTGGGGTTAATTTATAACTAAGAGTGCTCTAGTTTTGCAATACAGGACATGCTATAAAAATGGAAAGATGTTGCTTTCTGAGAGATGCGCCACC |
| 351 | > SP0127 (LVR_SP127) |
| | AGAATGAACATTGAACTTTGGACTATACCTGAGGGGTGAGGTAAACAACAGGACTATAAATGGCCCGGGAGGCGCCCTTTGGACCTTTTGCAATCCTGGCGCACTGAACCCTTGACCCCTGCCCTGCAGCCCCCGCAGCTTGCTG |
| | TTTGCCCACTCTATTTGCCCAGCCCCAGGGACAGAGCTGATCCTTGAACTCTTAAGTTCCACGGGCATATAAAACAGGGGCAAGGCACAGACTCATAGCAGAGCAATCACCACCAAGCCTGGAATAACTGCAGCCACC |
| 352 | > SP0127A1 (LVR SP127_A1) |
| | AGGTTAATTTTTAAAAAGCAGTCAAAAGTCCAAGTGGCCCTTGGCAGCATTTACTCTCTCTGTTTGCTCTGGTTAATAATCTCAGGAGCACAAACATTCCTGTACCAGAATGAACATTGAACTTTGGACTATACCTGAGGGGTGA |
| | GGTAAACAACAGGACTATAAATGGCCCGGGAGGCGCCCTTTGGACCTTTTGCAATCCTGGCGCACTGAACCCTTGACCCCTGCCCTGCAGCCCCCGCAGCTTGCTGTTTGCCCACTCTATTTGCCCAGCCCCAGGGACAGAGCTG |
| | ATCCTTGAACTCTTAAGTTCCACGGGCATATAAAACAGGGGCAAGGCACAGACTCATAGCAGAGCAATCACCACCAAGCCTGGAATAACTGCAGCCACC |
| 353 | > SP0127V1 (LVR SP127 V1) |
| | AAGCAAATATTTGTGGTTATGGATTAACTCGAACTGTTTGCCCACTCTATTTGCCCTGTACCAGAATGAACATTGAACTTTGGACTATACCTGAGGGGTGAGGTAAACAACAGGACTATAAATGGCCCGGGAGGCGCCCTTTGGA |
| | CCTTTTGCAATCCTGGCGCACTGAACCCTTGACCCCTGCCCTGCAGCCCCCGCAGCTTGCTGTTTGCCCACTCTATTTGCCCAGCCCCAGGGACAGAGCTGATCCTTGAACTCTTAAGTTCCACGGGCATATAAAACAGGGGCAA |
| | GGCACAGACTCATAGCAGAGCAATCACCACCAAGCCTGGAATAACTGCAGCCACC |
| 354 | > SP0127V2 (LVR_SP127_V2) |
| | GGCGCCCTTTGGACCTTTTGCAATCCTGGAGCAAACAGCAAACACTGTACCAGAATGAACATTGAACTTTGGACTATACCTGAGGGGTGAGGTAAACAACAGGACTATAAATGGCCCGGGAGGCGCCCTTTGGACCTTTTGCAAT |
| | CCTGGCGCACTGAACCCTTGACCCCTGCCCTGCAGCCCCCGCAGCTTGCTGTTTGCCCACTCTATTTGCCCAGCCCCAGGGACAGAGCTGATCCTTGAACTCTTAAGTTCCACGGGCATATAAAACAGGGGCAAGGCACAGACTC |
| | ATAGCAGAGCAATCACCACCAAGCCTGGAATAACTGCAGCCACC |
| 355 | > SP0128 |
| | AGAATGAACATTGAACTTTGGACTATACCTGAGGGGTGAGGTAAACAACAGGACTATAAATAGAATGAACATTGAACTTTGGACTATACCTGAGGGGTGAGGTAAACAACAGGACTATAAATCACTGAACCCTTGACCCCTGCCC |
| | TGCAGCCCCCGCAGCTTGCTGTTTGCCCACTCTATTTGCCCAGCCCCAGGGGCATATAAAACAGGGGCAAGGCACAGACTCATAGCAGAGCAATCACCACCAAGCCTGGAATAACTGCAGCCACC |
| 356 | >SP0131 (LVR_SP131) |
| | GGCCCGGGAGGCGCCCTTTGGACCTTTTGCAATCCTGGCGCACTGAACCCTTGACCCCTGCCCTGCAGCCCCCGCAGCTTGCTGTTTGCCCACTCTATTTGCCCAGCCCCAGCCCTGGAGAGTCCTTTAGCAGGGCAAAGTGCAA |
| | CATAGGCAGACCTTAAGGGATGACTCAGTAACAGATAAGCTTTGTGTGCCTGCAGGGCATATAAAACAGGGGCAAGGCACAGACTCATAGCAGAGCAATCACCACCAAGCCTGGAATAACTGCAGCCACC |
| 357 | > SP0132 (LVR_SP132) |
| | AGAATGAACATTGAACTTTGGACTATACCTGAGGGGTGAGGTAAACAACAGGACTATAAATGGCCCGGGAGGCGCCCTTTGGACCTTTTGCAATCCTGGCGCACTGAACCCTTGACCCCTGCCCTGCAGCCCCCGCAGCTTGCTG |
| | TTTGCCCACTCTATTTGCCCAGCCCCAGCCCTGGAGAGTCCTTTAGCAGGGCAAAGTGCAACATAGGCAGACCTTAAGGGATGACTCAGTAACAGATAAGCTTTGTGTGCCTGCAGGGCATATAAAACAGGGGCAAGGCACAGAC |
| | TCATAGCAGAGCAATCACCACCAAGCCTGGAATAACTGCAGCCACC |
| 358 | > SP0133 (LVR_SP133) |
| | AGAATGAACATTGAACTTTGGACTATACCTGAGGGGTGAGGTAAACAACAGGACTATAAATGGCCCGGGAGGCGCCCTTTGGACCTTTTGCAATCCTGGCGCTCTTTTGTTTTACATGAAGGGTCTGGCAGCCAAAGCAATCACT |
| | CAAAGTTCAAACCTTATCATTTTTTGCTTTGTTCCTCTTGGCCTTGGTTTTGTACATCAGCTTTGAAAATACCATCCCAGGGTTAATGCTGGGGTTAATTTATAACTAAGAGTGCTCTAGTTTTGCAATACAGGACATGCTATAA |
| | AAATGGAAAGATGTTGCTTTCTGAGAGATGCGCCACC |
| 359 | > SP0155 |
| | GCTGGTTTCTTATAAAACTGATGGAAGATACAAACACTATTAAAGAACTGTTTGCATGTTGCAAATGATGTCCAAAGTCCAAACATTGTTAATAATTAATACTCCAATAAACATCATGTCAGAATTTCTGTTTTCTTTTCCCTTT |
| | GAACCTTTGCAGGATTGCCACATCATCAGGACCACACCTTCATCAGGAATGAATATCCGATGACCTAATGATTCTGAGCTTGGCAAAGGTCTTATCTCCCAGCTCGCCCAGGCCCAGTGTTCCAGGAATGTGACCTTTGCTGCAG |
| | CAGCCGCTGGAGGGGGCAGAGGGGATGGGCTGGAGGTTGAGCAAACAGAGCAGCAGAAAAGGCAGTTCCTCTTCTCCAGTGCCCTCCTTCCCTGTCTCTGCCTCTCCCTCCCTTCCTCAGGCATCAGAGCGGAGACTTCAGGGAG |
| | ACCAGAGCCCAGCTTGCCAGGCACTGAGCTAGAAGCCCTGCCATG |
| 360 | > SP0158 |
| | ATTGGCATCTTCTATTGCTTTTCCTGGTGACTTCATTTTTCACTCTTGGCTAAAAATGGGTCTCTGATGATTTATTCTATCCTGGGTGTTGACAAGCTGAAGAAGTTGTGTGGGGCCTGCTGCCAGTAACCCTGGGTGACGAAGC |
| | GTGACTCACCACTCCGAGGTCAGTGGGGGGATGGAAGGCAGGGGAGTCAGCTGACAAGATCTGCTGCTTTGTCACCAGGCCTTCTGCCCGATGACCTAATGATTCTGAGCTTGGCAAAGGTCTTATCTCCCAGCTCGCCCAGGCC |
| | CAGTGTTCCAGGAATGTGACCTTTGCTGCAGCAGCCGCTGGAGGGGGCAGAGGGGATGGGCTGGAGGTTGAGCAAACAGAGCAGCAGAAAAGGCAGTTCCTCTTCTCCAGTGCCCTCCTTCCCTGTCTCTGCCTCTCCCTCCCTT |
| | CCTCAGGCATCAGAGCGGAGACTTCAGGGAGACCAGAGCCCAGCTTGCCAGGCACTGAGCTAGAAGCCCTGCCATG |
| 361 | > SP0163 |
| | TGAAATGCCTGCCATATATTAGTGCCCTGAAGTCCAAAGGTAGAGGAACCGAGTGTTTAAAAATTACTGTGGCTGTGGAGTCAACATGATGTAAAAAAACAAACATTTGGATAACACCAAGAAGCCAGATATGGTTGAAATGTTG |
| | ACTGGTTGACAAAAATAATTTGGGTTGCTTAATGGTGCACAAAGGTAATGCAAAACCGATGACCTAATGATTCTGAGCTTGGCAAAGGTCTTATCTCCCAGCTCGCCCAGGCCCAGTGTTCCAGGAATGTGACCTTTGCTGCAGC |
| | AGCCGCTGGAGGGGGCAGAGGGGATGGGCTGGAGGTTGAGCAAACAGAGCAGCAGAAAAGGCAGTTCCTCTTCTCCAGTGCCCTCCTTCCCTGTCTCTGCCTCTCCCTCCCTTCCTCAGGCATCAGAGCGGAGACTTCAGGGAGA |
| | CCAGAGCCCAGCTTGCCAGGCACTGAGCTAGAAGCCCTGCCATG |
| 362 | > SP0236 |
| | CAGGCTTTCACTTTCTCGCCAACTTACAAGGCCTTTCTGTGTAAACAATACCTGAACCTTTACCCCGTTGCCCGGCAACGGCCAGGTCTGTGCCAAGTGTTTGCCTTTGCAACAGCTTATCGGAAGCAAACAAGCTGAGGGGAAT |
| | TGAGCAAGAATTTCTGGGATACCAACAGCATAGGAGGAACAAAGGACGTAGAGGGAGGGTTGACTGTCTACACAGGACAAAGCCAATGATTAACCAAACCTCTTGCAGATTTAAATAGGATGGGAACTAGGAGTGGCAGCAATCC |
| | TTTCTTTCAGCTGGAGTGCTCCTCAGGAGCCAGCCCCACCCTTAGAAAAG |
| 93 | > SP0239 |
| | CAGGCTTTCACTTTCTCGCCAACTTACAAGGCCTTTCTGTGTAAACAATACCTGAACCTTTACCCCGTTGCCCGGCAACGGCCAGGTCTGTGCCAAGTGTTTGAGGTTAATTTTTAAAAAGCAGTCAAAAGTCCAAGTGGCCCTT |
| | GGCAGCATTTACTCTCTCTGTTTGCTCTGGTTAATAATCTCAGGAGCACAAACATTCCGGCCCGGGAGGCGCCCTTTGGACCTTTTGCAATCCTGGCGCACTGAACCCTTGACCCCTGCCCTGCAGCCCCCGCAGCTTGCTGTTT |
| | GCCCACTCTATTTGCCCAGCCCCAGCCCTGGAGAGTCCTTTAGCAGGGCAAAGTGCAACATAGGCAGACCTTAAGGGATGACTCAGTAACAGATAAGCTTTGTGTGCCTGCAGGGCATATAAAACAGGGGCAAGGCACAGACTCA |
| | TAGCAGAGCAATCACCACCAAGCCTGGAATAACTGCAGCCACC |
| 95 | > SP0240 |
| | CAGGCTTTCACTTTCTCGCCAACTTACAAGGCCTTTCTGTGTAAACAATACCTGAACCTTTACCCCGTTGCCCGGCAACGGCCAGGTCTGTGCCAAGTGTTTGCCGATGACCTAATGATTCTGAGCTTGGCAAAGGTCTTATCTC |
| | CCAGCTCGCCCAGGCCCAGTGTTCCAGGAATGTGACCTTTGCTGCAGCAGCCGCTGGAGGGGGCAGAGGGGATGGGCTGGAGGTTGAGCAAACAGAGCAGCAGAAAAGGCAGTTCCTCTTCTCCAGTGCCCTCCTTCCCTGTCTC |
| | TGCCTCTCCCTCCCTTCCTCAGGCATCAGAGCGGAGACTTCAGGGAGACCAGAGCCCAGCTTGCCAGGCACTGAGCTAGAAGCCCTGCC |
| 363 | > SP0241 |
| | AGGTTAATTTTTAAAAAGCAGTCAAAAGTCCAAGTGGCCCTTGGCAGCATTTACTCTCTCTGTTTGCTCTGGTTAATAATCTCAGGAGCACAAACATTCCGGCCCGGGAGGCGCCCTTTGGACCTTTTGCAATCCTGGCGCACTG |
| | AACCCTTGACCCCTGCCCTGCAGCCCCCGCAGCTTGCTGTTTGCCCACTCTATTTGCCCAGCCCCAGCCCTGGAGAGTCCTTTAGCAGGGCAAAGTGCAACATAGGCAGACCTTAAGGGATGACTCAGTAACAGATAAGCTTTGT |
| | GTGCCTGCACCGATGACCTAATGATTCTGAGCTTGGCAAAGGTCTTATCTCCCAGCTCGCCCAGGCCCAGTGTTCCAGGAATGTGACCTTTGCTGCAGCAGCCGCTGGAGGGGGCAGAGGGGATGGGCTGGAGGTTGAGCAAACA |
| | GAGCAGCAGAAAAGGCAGTTCCTCTTCTCCAGTGCCCTCCTTCCCTGTCTCTGCCTCTCCCTCCCTTCCTCAGGCATCAGAGCGGAGACTTCAGGGAGACCAGAGCCCAGCTTGCCAGGCACTGAGCTAGAAGCCCTGCC |
| 364 | > SP0242 |
| | AGGTTAATTTTTAAAAAGCAGTCAAAAGTCCAAGTGGCCCTTGGCAGCATTTACTCTCTCTGTTTGCTCTGGTTAATAATCTCAGGAGCACAAACATTCCGGCCCGGGAGGCGCCCTTTGGACCTTTTGCAATCCTGGCGCACTG |
| | AACCCTTGACCCCTGCCCTGCAGCCCCCGCAGCTTGCTGTTTGCCCACTCTATTTGCCCAGCCCCAGCCGATGACCTAATGATTCTGAGCTTGGCAAAGGTCTTATCTCCCAGCTCGCCCAGGCCCAGTGTTCCAGGAATGTGAC |
| | CTTTGCTGCAGCAGCCGCTGGAGGGGGCAGAGGGGATGGGCTGGAGGTTGAGCAAACAGAGCAGCAGAAAAGGCAGTTCCTCTTCTCCAGTGCCCTCCTTCCCTGTCTCTGCCTCTCCCTCCCTTCCTCAGGCATCAGAGCGGAG |
| | ACTTCAGGGAGACCAGAGCCCAGCTTGCCAGGCACTGAGCTAGAAGCCCTGCC |
| 365 | > SP0243 |
| | TGAAATGCCTGCCATATATTAGTGCCCTGAAGTCCAAAGGTAGAGGAACCGAGTGTTTAAAAATTACTGTGGCTGTGGAGTCAACATGATGTAAAAAAACAAACATTTGGATAACACCAAGAAGCCAGATATGGTTGAAATGTTG |
| | ACTGGTTGACAAAAATAATTTGGGTTGCTTAATGGTGCACAAAGGTAATGCAAAAAGGTTAATTTTTAAAAAGCAGTCAAAAGTCCAAGTGGCCCTTGGCAGCATTTACTCTCTCTGTTTGCTCTGGTTAATAATCTCAGGAGCA |
| | CAAACATTCCGGCCCGGGAGGCGCCCTTTGGACCTTTTGCAATCCTGGCGCACTGAACCCTTGACCCCTGCCCTGCAGCCCCCGCAGCTTGCTGTTTGCCCACTCTATTTGCCCAGCCCCAGCCCTGGAGAGTCCTTTAGCAGGG |
| | CAAAGTGCAACATAGGCAGACCTTAAGGGATGACTCAGTAACAGATAAGCTTTGTGTGCCTGCAGGGCATATAAAACAGGGGCAAGGCACAGACTCATAGCAGAGCAATCACCACCAAGCCTGGAATAACTGCAGCCACC |
| 366 | > SP0244 |
| | AGGTTAATTTTTAAAAAGCAGTCAAAAGTCCAAGTGGCCCTTGGCAGCATTTACTCTCTCTGTTTGCTCTGGTTAATAATCTCAGGAGCACAAACATTCCGGCCCGGGAGGCGCCCTTTGGACCTTTTGCAATCCTGGCGCACTG |
| | AACCCTTGACCCCTGCCCTGCAGCCCCCGCAGCTTGCTGTTTGCCCACTCTATTTGCCCAGCCCCAGTGAAATGCCTGCCATATATTAGTGCCCTGAAGTCCAAAGGTAGAGGAACCGAGTGTTTAAAAATTACTGTGGCTGTGG |
| | AGTCAACATGATGTAAAAAAACAAACATTTGGATAACACCAAGAAGCCAGATATGGTTGAAATGTTGACTGGTTGACAAAAATAATTTGGGTTGCTTAATGGTGCACAAAGGTAATGCAAAACCGATGACCTAATGATTCTGAGC |
| | TTGGCAAAGGTCTTATCTCCCAGCTCGCCCAGGCCCAGTGTTCCAGGAATGTGACCTTTGCTGCAGCAGCCGCTGGAGGGGGCAGAGGGGATGGGCTGGAGGTTGAGCAAACAGAGCAGCAGAAAAGGCAGTTCCTCTTCTCCAG |
| | TGCCCTCCTTCCCTGTCTCTGCCTCTCCCTCCCTTCCTCAGGCATCAGAGCGGAGACTTCAGGGAGACCAGAGCCCAGCTTGCCAGGCACTGAGCTAGAAGCCCTGCC |
| 96 | > SP0246 |
| | AGGTTAATTTTTAAAAAGCAGTCAAAAGTCCAAGTGGCCCTTGGCAGCATTTACTCTCTCTGTTTGCTCTGGTTAATAATCTCAGGAGCACAAACATTCCGGCCCGGGAGGCGCCCTTTGGACCTTTTGCAATCCTGGCGCACTG |
| | AACCCTTGACCCCTGCCCTGCAGCCCCCGCAGCTTGCTGTTTGCCCACTCTATTTGCCCAGCCCCAGGGGCATATAAAACAGGGGCAAGGCACAGACTCATAGCAGAGCAATCACCACCAAGCCTGGAATAACTGCAGCCACC |
| 367 | > SP0247 |
| | GCTGGTTTCTTATAAAACTGATGGAAGATACAAACACTATTAAAGAACTGTTTGCATGTTGCAAATGATGTCCAAAGTCCAAACATTGTTAATAATTAATACTCCAATAAACATCATGTCAGAATTTCTGTTTTCTTTTCCCTTT |
| | GAACCTTTGCAGGATTGCCACATCATCAGGACCACACCTTCATCAGGAATGAATATCAGGCTTTCACTTTCTCGCCAACTTACAAGGCCTTTCTGTGTAAACAATACCTGAACCTTTACCCCGTTGCCCGGCAACGGCCAGGTCT |
| | GTGCCAAGTGTTTGCCGATGACCTAATGATTCTGAGCTTGGCAAAGGTCTTATCTCCCAGCTCGCCCAGGCCCAGTGTTCCAGGAATGTGACCTTTGCTGCAGCAGCCGCTGGAGGGGGCAGAGGGGATGGGCTGGAGGTTGAGC |
| | AAACAGAGCAGCAGAAAAGGCAGTTCCTCTTCTCCAGTGCCCTCCTTCCCTGTCTCTGCCTCTCCCTCCCTTCCTCAGGCATCAGAGCGGAGACTTCAGGGAGACCAGAGCCCAGCTTGCCAGGCACTGAGCTAGAAGCCCTGCC |
| 368 | > SP0248 |
| | AGGTTAATTTTTAAAAAGCAGTCAAAAGTCCAAGTGGCCCTTGGCAGCATTTACTCTCTCTGTTTGCTCTGGTTAATAATCTCAGGAGCACAAACATTCCGGCCCGGGAGGCGCCCTTTGGACCTTTTGCAATCCTGGCGCACTG |
| | AACCCTTGACCCCTGCCCTGCAGCCCCCGCAGCTTGCTGTTTGCCCACTCTATTTGCCCAGCCCCAGCAGGCTTTCACTTTCTCGCCAACTTACAAGGCCTTTCTGTGTAAACAATACCTGAACCTTTACCCCGTTGCCCGGCAA |
| | CGGCCAGGTCTGTGCCAAGTGTTTGGGGCATATAAAACAGGGGCAAGGCACAGACTCATAGCAGAGCAATCACCACCAAGCCTGGAATAACTGCAGCCACC |
| 369 | > SP0249 |
| | AGGTTAATTTTTAAAAAGCAGTCAAAAGTCCAAGTGGCCCTTGGCAGCATTTACTCTCTCTGTTTGCTCTGGTTAATAATCTCAGGAGCACAAACATTCCGGCCCGGGAGGCGCCCTTTGGACCTTTTGCAATCCTGGCGCACTG |
| | AACCCTTGACCCCTGCCCTGCAGCCCCCGCAGCTTGCTGTTTGCCCACTCTATTTGCCCAGCCCCAGCAGGCTTTCACTTTCTCGCCAACTTACAAGGCCTTTCTGTGTAAACAATACCTGAACCTTTACCCCGTTGCCCGGCAA |
| | CGGCCAGGTCTGTGCCAAGTGTTTGGGGCGACTCAGATCCCAGCCAGTGGACTTAGCCCCTGTTTGCTCCTCCGATAACTGGGGTGACCTTGGTTAATATTCACCAGCAGCCTCCCCCGTTGCCCCTCTGGATCCACTGCTTAAA |
| | TACGGACGAGGACAGGGCCCTGTCTCCTCAGCTTCAGGCACCACCACTGACCTGGGACAGTGAATCGCCACC |
| 370 | > SP0250 |
| | AGGTTAATTTTTAAAAAGCAGTCAAAAGTCCAAGTGGCCCTTGGCAGCATTTACTCTCTCTGTTTGCTCTGGTTAATAATCTCAGGAGCACAAACATTCCGGCCCGGGAGGCGCCCTTTGGACCTTTTGCAATCCTGGCGCACTG |
| | AACCCTTGACCCCTGCCCTGCAGCCCCCGCAGCTTGCTGTTTGCCCACTCTATTTGCCCAGCCCCAGGCTGGTTTCTTATAAAACTGATGGAAGATACAAACACTATTAAAGAACTGTTTGCATGTTGCAAATGATGTCCAAAGT |
| | CCAAACATTGTTAATAATTAATACTCCAATAAACATCATGTCAGAATTTCTGTTTTCTTTTCCCTTTGAACCTTTGCAGGATTGCCACATCATCAGGACCACACCTTCATCAGGAATGAATATCCGATGACCTAATGATTCTGAG |
| | CTTGGCAAAGGTCTTATCTCCCAGCTCGCCCAGGCCCAGTGTTCCAGGAATGTGACCTTTGCTGCAGCAGCCGCTGGAGGGGGCAGAGGGGATGGGCTGGAGGTTGAGCAAACAGAGCAGCAGAAAAGGCAGTTCCTCTTCTCCA |
| | GTGCCCTCCTTCCCTGTCTCTGCCTCTCCCTCCCTTCCTCAGGCATCAGAGCGGAGACTTCAGGGAGACCAGAGCCCAGCTTGCCAGGCACTGAGCTAGAAGCCCTGCC |
| 371 | > SP0251 |
| | AGGTTAATTTTTAAAAAGCAGTCAAAAGTCCAAGTGGCCCTTGGCAGCATTTACTCTCTCTGTTTGCTCTGGTTAATAATCTCAGGAGCACAAACATTCCGGCCCGGGAGGCGCCCTTTGGACCTTTTGCAATCCTGGCGCACTG |
| | AACCCTTGACCCCTGCCCTGCAGCCCCCGCAGCTTGCTGTTTGCCCACTCTATTTGCCCAGCCCCAGAAGCAAATATTTGTGGTTATGGATTAACTCGAACTGTTTGCCCACTCTATTTGCCCGGGCATATAAAACAGGGGCAAG |
| | GCACAGACTCATAGCAGAGCAATCACCACCAAGCCTGGAATAACTGCAGCCACC |
| 372 | > SP0252 |
| | AAGCAAATATTTGTGGTTATGGATTAACTCGAACTGTTTGCCCACTCTATTTGCCCCAGGCTTTCACTTTCTCGCCAACTTACAAGGCCTTTCTGTGTAAACAATACCTGAACCTTTACCCCGTTGCCCGGCAACGGCCAGGTCT |
| | GTGCCAAGTGTTTGCCTTTGCAACAGCTTATCGGAAGCAAACAAGCTGAGGGGAATTGAGCAAGAATTTCTGGGATACCAACAGCATAGGAGGAACAAAGGACGTAGAGGGAGGGTTGACTGTCTACACAGGACAAAGCCAATGA |
| | TTAACCAAACCTCTTGCAGATTTAAATAGGATGGGAACTAGGAGTGGCAGCAATCCTTTCTTTCAGCTGGAGTGCTCCTCAGGAGCCAGCCCCACCCTTAGAAAAGCCACC |
| 373 | > SP0253 |
| | AAGCAAATATTTGTGGTTATGGATTAACTCGAACTGTTTGCCCACTCTATTTGCCCCAGGCTTTCACTTTCTCGCCAACTTACAAGGCCTTTCTGTGTAAACAATACCTGAACCTTTACCCCGTTGCCCGGCAACGGCCAGGTCT |
| | GTGCCAAGTGTTTGCCGATGACCTAATGATTCTGAGCTTGGCAAAGGTCTTATCTCCCAGCTCGCCCAGGCCCAGTGTTCCAGGAATGTGACCTTTGCTGCAGCAGCCGCTGGAGGGGGCAGAGGGGATGGGCTGGAGGTTGAGC |
| | AAACAGAGCAGCAGAAAAGGCAGTTCCTCTTCTCCAGTGCCCTCCTTCCCTGTCTCTGCCTCTCCCTCCCTTCCTCAGGCATCAGAGCGGAGACTTCAGGGAGACCAGAGCCCAGCTTGCCAGGCACTGAGCTAGAAGCCCTGCC |
| 374 | > SP0254 |
| | AGGTTAATTTTTAAAAAGCAGTCAAAAGTCCAAGTGGCCCTTGGCAGCATTTACTCTCTCTGTTTGCTCTGGTTAATAATCTCAGGAGCACAAACATTCCGGCCCGGGAGGCGCCCTTTGGACCTTTTGCAATCCTGGCGCAGGC |
| | TTTCACTTTCTCGCCAACTTACAAGGCCTTTCTGTGTAAACAATACCTGAACCTTTACCCCGTTGCCCGGCAACGGCCAGGTCTGTGCCAAGTGTTTGCCTTTGCAACAGCTTATCGGAAGCAAACAAGCTGAGGGGAATTGAGC |
| | AAGAATTTCTGGGATACCAACAGCATAGGAGGAACAAAGGACGTAGAGGGAGGGTTGACTGTCTACACAGGACAAAGCCAATGATTAACCAAACCTCTTGCAGATTTAAATAGGATGGGAACTAGGAGTGGCAGCAATCCTTTCT |
| | TTCAGCTGGAGTGCTCCTCAGGAGCCAGCCCCACCCTTAGAAAAGCCACC |
| 375 | > SP0255 |
| | AGGTTAATTTTTAAAAAGCAGTCAAAAGTCCAAGTGGCCCTTGGCAGCATTTACTCTCTCTGTTTGCTCTGGTTAATAATCTCAGGAGCACAAACATTCCGGCCCGGGAGGCGCCCTTTGGACCTTTTGCAATCCTGGCGCAGGC |
| | TTTCACTTTCTCGCCAACTTACAAGGCCTTTCTGTGTAAACAATACCTGAACCTTTACCCCGTTGCCCGGCAACGGCCAGGTCTGTGCCAAGTGTTTGCCGATGACCTAATGATTCTGAGCTTGGCAAAGGTCTTATCTCCCAGC |
| | TCGCCCAGGCCCAGTGTTCCAGGAATGTGACCTTTGCTGCAGCAGCCGCTGGAGGGGGCAGAGGGGATGGGCTGGAGGTTGAGCAAACAGAGCAGCAGAAAAGGCAGTTCCTCTTCTCCAGTGCCCTCCTTCCCTGTCTCTGCCT |
| | CTCCCTCCCTTCCTCAGGCATCAGAGCGGAGACTTCAGGGAGACCAGAGCCCAGCTTGCCAGGCACTGAGCTAGAAGCCCTGCC |
| 376 | > SP0256 |
| | ATTGGCATCTTCTATTGCTTTTCCTGGTGACTTCATTTTTCACTCTTGGCTAAAAATGGGTCTCTGATGATTTATTCTATCCTGGGTGTTGACAAGCTGAAGAAGTTGTGTGGGGCCTGCTGCCAGTAACCCTGGGTGACGAAGC |
| | GTGACTCACCACTCCGAGGTCAGTGGGGGGATGGAAGGCAGGGGAGTCAGCTGACAAGATCTGCTGCTTTGTCACCAGGCCTTCTGCCAGGCTTTCACTTTCTCGCCAACTTACAAGGCCTTTCTGTGTAAACAATACCTGAACC |
| | TTTACCCCGTTGCCCGGCAACGGCCAGGTCTGTGCCAAGTGTTTGCCGATGACCTAATGATTCTGAGCTTGGCAAAGGTCTTATCTCCCAGCTCGCCCAGGCCCAGTGTTCCAGGAATGTGACCTTTGCTGCAGCAGCCGCTGGA |
| | GGGGGCAGAGGGGATGGGCTGGAGGTTGAGCAAACAGAGCAGCAGAAAAGGCAGTTCCTCTTCTCCAGTGCCCTCCTTCCCTGTCTCTGCCTCTCCCTCCCTTCCTCAGGCATCAGAGCGGAGACTTCAGGGAGACCAGAGCCCA |
| | GCTTGCCAGGCACTGAGCTAGAAGCCCTGCC |
| 377 | > SP0257 |
| | CAGGCTTTCACTTTCTCGCCAACTTACAAGGCCTTTCTGTGTAAACAATACCTGAACCTTTACCCCGTTGCCCGGCAACGGCCAGGTCTGTGCCAAGTGTTTGGCTGGTTTCTTATAAAACTGATGGAAGATACAAACACTATTA |
| | AAGAACTGTTTGCATGTTGCAAATGATGTCCAAAGTCCAAACATTGTTAATAATTAATACTCCAATAAACATCATGTCAGAATTTCTGTTTTCTTTTCCCTTTGAACCTTTGCAGGATTGCCACATCATCAGGACCACACCTTCA |
| | TCAGGAATGAATATCCGATGACCTAATGATTCTGAGCTTGGCAAAGGTCTTATCTCCCAGCTCGCCCAGGCCCAGTGTTCCAGGAATGTGACCTTTGCTGCAGCAGCCGCTGGAGGGGGCAGAGGGGATGGGCTGGAGGTTGAGC |
| | AAACAGAGCAGCAGAAAAGGCAGTTCCTCTTCTCCAGTGCCCTCCTTCCCTGTCTCTGCCTCTCCCTCCCTTCCTCAGGCATCAGAGCGGAGACTTCAGGGAGACCAGAGCCCAGCTTGCCAGGCACTGAGCTAGAAGCCCTGCC |
| 378 | > SP0258 |
| | CTGTTTGCTGCTTGCAATGTTTGCCCATTTTAGGGAGGTTAATTTTTAAAAAGCAGTCAAAAGTCCAAGTGGCCCTTGGCAGCATTTACTCTCTCTGTTTGCTCTGGTTAATAATCTCAGGAGCACAAACATTCCGGCCCGGGAG |
| | GCGCCCTTTGGACCTTTTGCAATCCTGGCGCACTGAACCCTTGACCCCTGCCCTGCAGCCCCCGCAGCTTGCTGTTTGCCCACTCTATTTGCCCAGCCCCAGCCCTGGAGAGTCCTTTAGCAGGGCAAAGTGCAACATAGGCAGA |
| | CCTTAAGGGATGACTCAGTAACAGATAAGCTTTGTGTGCCTGCAGGGCATATAAAACAGGGGCAAGGCACAGACTCATAGCAGAGCAATCACCACCAAGCCTGGAATAACTGCAGCCACC |
| 379 | > SP0259 |
| | CTGTTTGCTGCTTGCAATGTTTGCCCATTTTAGGGGCTGGTTTCTTATAAAACTGATGGAAGATACAAACACTATTAAAGAACTGTTTGCATGTTGCAAATGATGTCCAAAGTCCAAACATTGTTAATAATTAATACTCCAATAA |
| | ACATCATGTCAGAATTTCTGTTTTCTTTTCCCTTTGAACCTTTGCAGGATTGCCACATCATCAGGACCACACCTTCATCAGGAATGAATATCCGATGACCTAATGATTCTGAGCTTGGCAAAGGTCTTATCTCCCAGCTCGCCCA |
| | GGCCCAGTGTTCCAGGAATGTGACCTTTGCTGCAGCAGCCGCTGGAGGGGGCAGAGGGGATGGGCTGGAGGTTGAGCAAACAGAGCAGCAGAAAAGGCAGTTCCTCTTCTCCAGTGCCCTCCTTCCCTGTCTCTGCCTCTCCCTC |
| | CCTTCCTCAGGCATCAGAGCGGAGACTTCAGGGAGACCAGAGCCCAGCTTGCCAGGCACTGAGCTAGAAGCCCTGCC |
| 380 | > SP0264 |
| | CAGGCTTTCACTTTCTCGCCAACTTACAAGGCCTTTCTGTGTAAACAATACCTGAACCTTTACCCCGTTGCCCGGCAACGGCCAGGTCTGTGCCAAGTGTTTGCCTTTGCAACAGCTTATCGGAAGCAAACAAGCTGAGGGGAAT |
| | TGAGCAAGAATTTCTGGGATACCAACAGCATAGGAGGAACAAAGGACGTAGAGGGAGGGTTGACTGTCTACACAGGACAAAGCCAATGATTAACCAAACCTCTTGCAGATTTAAATAGGATGGGAACTAGGAGTGGCAGCAATCC |
| | TTTCTTTCAGCTGGAGTGCTCCTCAGGagCCAGCCCCACCCTTAGAAAAGCCACC |
| 94 | > SP0265 (LVR_SP131_A1) |
| | AGGTTAATTTTTAAAAAGCAGTCAAAAGTCCAAGTGGCCCTTGGCAGCATTTACTCTCTCTGTTTGCTCTGGTTAATAATCTCAGGAGCACAAACATTCCTGTACCGGCCCGGGAGGCGCCCTTTGGACCTTTTGCAATCCTGGC |
| | GCACTGAACCCTTGACCCCTGCCCTGCAGCCCCCGCAGCTTGCTGTTTGCCCACTCTATTTGCCCAGCCCCAGCCCTGGAGAGTCCTTTAGCAGGGCAAAGTGCAACATAGGCAGACCTTAAGGGATGACTCAGTAACAGATAAG |
| | CTTTGTGTGCCTGCAGGGCATATAAAACAGGGGCAAGGCACAGACTCATAGCAGAGCAATCACCACCAAGCCTGGAATAACTGCAGCCACC |
| 381 | > SP0266 (LVR_SP131 V1) |
| | AAGCAAATATTTGTGGTTATGGATTAACTCGAACTGTTTGCCCACTCTATTTGCCCTGTACCGGCCCGGGAGGCGCCCTTTGGACCTTTTGCAATCCTGGCGCACTGAACCCTTGACCCCTGCCCTGCAGCCCCCGCAGCTTGCT |
| | GTTTGCCCACTCTATTTGCCCAGCCCCAGCCCTGGAGAGTCCTTTAGCAGGGCAAAGTGCAACATAGGCAGACCTTAAGGGATGACTCAGTAACAGATAAGCTTTGTGTGCCTGCAGGGCATATAAAACAGGGGCAAGGCACAGA |
| | CTCATAGCAGAGCAATCACCACCAAGCCTGGAATAACTGCAGCCACC |
| 382 | > SP0267 (LVR_SP131_V2) |
| | GGCGCCCTTTGGACCTTTTGCAATCCTGGAGCAAACAGCAAACACTGTACCGGCCCGGGAGGCGCCCTTTGGACCTTTTGCAATCCTGGCGCACTGAACCCTTGACCCCTGCCCTGCAGCCCCCGCAGCTTGCTGTTTGCCCACT |
| | CTATTTGCCCAGCCCCAGCCCTGGAGAGTCCTTTAGCAGGGCAAAGTGCAACATAGGCAGACCTTAAGGGATGACTCAGTAACAGATAAGCTTTGTGTGCCTGCAGGGCATATAAAACAGGGGCAAGGCACAGACTCATAGCAGA |
| | GCAATCACCACCAAGCCTGGAATAACTGCAGCCACC |
| 383 | > SP0268 (LVR 132_A1) |
| | AGGTTAATTTTTAAAAAGCAGTCAAAAGTCCAAGTGGCCCTTGGCAGCATTTACTCTCTCTGTTTGCTCTGGTTAATAATCTCAGGAGCACAAACATTCCTGTACCAGAATGAACATTGAACTTTGGACTATACCTGAGGGGTGA |
| | GGTAAACAACAGGACTATAAATGGCCCGGGAGGCGCCCTTTGGACCTTTTGCAATCCTGGCGCACTGAACCCTTGACCCCTGCCCTGCAGCCCCCGCAGCTTGCTGTTTGCCCACTCTATTTGCCCAGCCCCAGCCCTGGAGAGT |
| | CCTTTAGCAGGGCAAAGTGCAACATAGGCAGACCTTAAGGGATGACTCAGTAACAGATAAGCTTTGTGTGCCTGCAGGGCATATAAAACAGGGGCAAGGCACAGACTCATAGCAGAGCAATCACCACCAAGCCTGGAATAACTGC |
| | AGCCACC |
| 384 | > SP0269 (LVR_132_V1) |
| | AAGCAAATATTTGTGGTTATGGATTAACTCGAACTGTTTGCCCACTCTATTTGCCCTGTACCAGAATGAACATTGAACTTTGGACTATACCTGAGGGGTGAGGTAAACAACAGGACTATAAATGGCCCGGGAGGCGCCCTTTGGA |
| | CCTTTTGCAATCCTGGCGCACTGAACCCTTGACCCCTGCCCTGCAGCCCCCGCAGCTTGCTGTTTGCCCACTCTATTTGCCCAGCCCCAGCCCTGGAGAGTCCTTTAGCAGGGCAAAGTGCAACATAGGCAGACCTTAAGGGATG |
| | ACTCAGTAACAGATAAGCTTTGTGTGCCTGCAGGGCATATAAAACAGGGGCAAGGCACAGACTCATAGCAGAGCAATCACCACCAAGCCTGGAATAACTGCAGCCACC |
| 385 | > SP0270 (LVR_132_V2) |
| | GGCGCCCTTTGGACCTTTTGCAATCCTGGAGCAAACAGCAAACACTGTACCAGAATGAACATTGAACTTTGGACTATACCTGAGGGGTGAGGTAAACAACAGGACTATAAATGGCCCGGGAGGCGCCCTTTGGACCTTTTGCAAT |
| | CCTGGCGCACTGAACCCTTGACCCCTGCCCTGCAGCCCCCGCAGCTTGCTGTTTGCCCACTCTATTTGCCCAGCCCCAGCCCTGGAGAGTCCTTTAGCAGGGCAAAGTGCAACATAGGCAGACCTTAAGGGATGACTCAGTAACA |
| | GATAAGCTTTGTGTGCCTGCAGGGCATATAAAACAGGGGCAAGGCACAGACTCATAGCAGAGCAATCACCACCAAGCCTGGAATAACTGCAGCCACC |
| 386 | > SP0271 (LVR 133_A1) |
| | AGGTTAATTTTTAAAAAGCAGTCAAAAGTCCAAGTGGCCCTTGGCAGCATTTACTCTCTCTGTTTGCTCTGGTTAATAATCTCAGGAGCACAAACATTCCTGTACCAGAATGAACATTGAACTTTGGACTATACCTGAGGGGTGA |
| | GGTAAACAACAGGACTATAAATGGCCCGGGAGGCGCCCTTTGGACCTTTTGCAATCCTGGCGCTCTTTTGTTTTACATGAAGGGTCTGGCAGCCAAAGCAATCACTCAAAGTTCAAACCTTATCATTTTTTGCTTTGTTCCTCTT |
| | GGCCTTGGTTTTGTACATCAGCTTTGAAAATACCATCCCAGGGTTAATGCTGGGGTTAATTTATAACTAAGAGTGCTCTAGTTTTGCAATACAGGACATGCTATAAAAATGGAAAGATGTTGCTTTCTGAGAGATGCGCCACC |
| 387 | > SP0272 (LVR 133_V1) |
| | AAGCAAATATTTGTGGTTATGGATTAACTCGAACTGTTTGCCCACTCTATTTGCCCTGTACCAGAATGAACATTGAACTTTGGACTATACCTGAGGGGTGAGGTAAACAACAGGACTATAAATGGCCCGGGAGGCGCCCTTTGGA |
| | CCTTTTGCAATCCTGGCGCTCTTTTGTTTTACATGAAGGGTCTGGCAGCCAAAGCAATCACTCAAAGTTCAAACCTTATCATTTTTTGCTTTGTTCCTCTTGGCCTTGGTTTTGTACATCAGCTTTGAAAATACCATCCCAGGGT |
| | TAATGCTGGGGTTAATTTATAACTAAGAGTGCTCTAGTTTTGCAATACAGGACATGCTATAAAAATGGAAAGATGTTGCTTTCTGAGAGATGCGCCACC |
| 388 | > SP0273 (LVR_133_V2) |
| | GGCGCCCTTTGGACCTTTTGCAATCCTGGAGCAAACAGCAAACACTGTACCAGAATGAACATTGAACTTTGGACTATACCTGAGGGGTGAGGTAAACAACAGGACTATAAATGGCCCGGGAGGCGCCCTTTGGACCTTTTGCAAT |
| | CCTGGCGCTCTTTTGTTTTACATGAAGGGTCTGGCAGCCAAAGCAATCACTCAAAGTTCAAACCTTATCATTTTTTGCTTTGTTCCTCTTGGCCTTGGTTTTGTACATCAGCTTTGAAAATACCATCCCAGGGTTAATGCTGGGG |
| | TTAATTTATAACTAAGAGTGCTCTAGTTTTGCAATACAGGACATGCTATAAAAATGGAAAGATGTTGCTTTCTGAGAGATGCGCCACC |
| 389 | > SP0256 |
| | ATTGGCATCTTCTATTGCTTTTCCTGGTGACTTCATTTTTCACTCTTGGCTAAAAATGGGTCTCTGATGATTTATTCTATCCTGGGTGTTGACAAGCTGAAGAAGTTGTGTGGGGCCTGCTGCCAGTAACCCTGGGTGACGAAGC |
| | GTGACTCACCACTCCGAGGTCAGTGGGGGGATGGAAGGCAGGGGAGTCAGCTGACAAGATCTGCTGCTTTGTCACCAGGCCTTCTGCCAGGCTTTCACTTTCTCGCCAACTTACAAGGCCTTTCTGTGTAAACAATACCTGAACC |
| | TTTACCCCGTTGCCCGGCAACGGCCAGGTCTGTGCCAAGTGTTTGCCGATGACCTAATGATTCTGAGCTTGGCAAAGGTCTTATCTCCCAGCTCGCCCAGGCCCAGTGTTCCAGGAATGTGACCTTTGCTGCAGCAGCCGCTGGA |
| | GGGGGCAGAGGGGATGGGCTGGAGGTTGAGCAAACAGAGCAGCAGAAAAGGCAGTTCCTCTTCTCCAGTGCCCTCCTTCCCTGTCTCTGCCTCTCCCTCCCTTCCTCAGGCATCAGAGCGGAGACTTCAGGGAGACCAGAGCCCA |
| | GCTTGCCAGGCACTGAGCTAGAAGCCCTGCC |
| 390 | > SP0257 |
| | CAGGCTTTCACTTTCTCGCCAACTTACAAGGCCTTTCTGTGTAAACAATACCTGAACCTTTACCCCGTTGCCCGGCAACGGCCAGGTCTGTGCCAAGTGTTTGGCTGGTTTCTTATAAAACTGATGGAAGATACAAACACTATTA |
| | AAGAACTGTTTGCATGTTGCAAATGATGTCCAAAGTCCAAACATTGTTAATAATTAATACTCCAATAAACATCATGTCAGAATTTCTGTTTTCTTTTCCCTTTGAACCTTTGCAGGATTGCCACATCATCAGGACCACACCTTCA |
| | TCAGGAATGAATATCCGATGACCTAATGATTCTGAGCTTGGCAAAGGTCTTATCTCCCAGCTCGCCCAGGCCCAGTGTTCCAGGAATGTGACCTTTGCTGCAGCAGCCGCTGGAGGGGGCAGAGGGGATGGGCTGGAGGTTGAGC |
| | AAACAGAGCAGCAGAAAAGGCAGTTCCTCTTCTCCAGTGCCCTCCTTCCCTGTCTCTGCCTCTCCCTCCCTTCCTCAGGCATCAGAGCGGAGACTTCAGGGAGACCAGAGCCCAGCTTGCCAGGCACTGAGCTAGAAGCCCTGCC |
| 391 | > SP0258 |
| | CTGTTTGCTGCTTGCAATGTTTGCCCATTTTAGGGAGGTTAATTTTTAAAAAGCAGTCAAAAGTCCAAGTGGCCCTTGGCAGCATTTACTCTCTCTGTTTGCTCTGGTTAATAATCTCAGGAGCACAAACATTCCGGCCCGGGAG |
| | GCGCCCTTTGGACCTTTTGCAATCCTGGCGCACTGAACCCTTGACCCCTGCCCTGCAGCCCCCGCAGCTTGCTGTTTGCCCACTCTATTTGCCCAGCCCCAGCCCTGGAGAGTCCTTTAGCAGGGCAAAGTGCAACATAGGCAGA |
| | CCTTAAGGGATGACTCAGTAACAGATAAGCTTTGTGTGCCTGCAGGGCATATAAAACAGGGGCAAGGCACAGACTCATAGCAGAGCAATCACCACCAAGCCTGGAATAACTGCAGCCACC |
| 392 | > SP0259 |
| | CTGTTTGCTGCTTGCAATGTTTGCCCATTTTAGGGGCTGGTTTCTTATAAAACTGATGGAAGATACAAACACTATTAAAGAACTGTTTGCATGTTGCAAATGATGTCCAAAGTCCAAACATTGTTAATAATTAATACTCCAATAA |
| | ACATCATGTCAGAATTTCTGTTTTCTTTTCCCTTTGAACCTTTGCAGGATTGCCACATCATCAGGACCACACCTTCATCAGGAATGAATATCCGATGACCTAATGATTCTGAGCTTGGCAAAGGTCTTATCTCCCAGCTCGCCCA |
| | GGCCCAGTGTTCCAGGAATGTGACCTTTGCTGCAGCAGCCGCTGGAGGGGGCAGAGGGGATGGGCTGGAGGTTGAGCAAACAGAGCAGCAGAAAAGGCAGTTCCTCTTCTCCAGTGCCCTCCTTCCCTGTCTCTGCCTCTCCCTC |
| | CCTTCCTCAGGCATCAGAGCGGAGACTTCAGGGAGACCAGAGCCCAGCTTGCCAGGCACTGAGCTAGAAGCCCTGCC |
| 393 | > SP0264 |
| | CAGGCTTTCACTTTCTCGCCAACTTACAAGGCCTTTCTGTGTAAACAATACCTGAACCTTTACCCCGTTGCCCGGCAACGGCCAGGTCTGTGCCAAGTGTTTGCCTTTGCAACAGCTTATCGGAAGCAAACAAGCTGAGGGGAAT |
| | TGAGCAAGAATTTCTGGGATACCAACAGCATAGGAGGAACAAAGGACGTAGAGGGAGGGTTGACTGTCTACACAGGACAAAGCCAATGATTAACCAAACCTCTTGCAGATTTAAATAGGATGGGAACTAGGAGTGGCAGCAATCC |
| | TTTCTTTCAGCTGGAGTGCTCCTCAGGAGCCAGCCCCACCCTTAGAAAAGCCACC |
| 394 | >SP0265 (LVR_SP131_A1) |
| | AGGTTAATTTTTAAAAAGCAGTCAAAAGTCCAAGTGGCCCTTGGCAGCATTTACTCTCTCTGTTTGCTCTGGTTAATAATCTCAGGAGCACAAACATTCCTGTACCGGCCCGGGAGGCGCCCTTTGGACCTTTTGCAATCCTGGC |
| | GCACTGAACCCTTGACCCCTGCCCTGCAGCCCCCGCAGCTTGCTGTTTGCCCACTCTATTTGCCCAGCCCCAGCCCTGGAGAGTCCTTTAGCAGGGCAAAGTGCAACATAGGCAGACCTTAAGGGATGACTCAGTAACAGATAAG |
| | CTTTGTGTGCCTGCAGGGCATATAAAACAGGGGCAAGGCACAGACTCATAGCAGAGCAATCACCACCAAGCCTGGAATAACTGCAGCCACC |
| 395 | > SP0266 (LVR SP131 V1) |
| | AAGCAAATATTTGTGGTTATGGATTAACTCGAACTGTTTGCCCACTCTATTTGCCCTGTACCGGCCCGGGAGGCGCCCTTTGGACCTTTTGCAATCCTGGCGCACTGAACCCTTGACCCCTGCCCTGCAGCCCCCGCAGCTTGCT |
| | GTTTGCCCACTCTATTTGCCCAGCCCCAGCCCTGGAGAGTCCTTTAGCAGGGCAAAGTGCAACATAGGCAGACCTTAAGGGATGACTCAGTAACAGATAAGCTTTGTGTGCCTGCAGGGCATATAAAACAGGGGCAAGGCACAGA |
| | CTCATAGCAGAGCAATCACCACCAAGCCTGGAATAACTGCAGCCACC |
| 396 | > SP0267 (LVR_SP131_V2) |
| | GGCGCCCTTTGGACCTTTTGCAATCCTGGAGCAAACAGCAAACACTGTACCGGCCCGGGAGGCGCCCTTTGGACCTTTTGCAATCCTGGCGCACTGAACCCTTGACCCCTGCCCTGCAGCCCCCGCAGCTTGCTGTTTGCCCACT |
| | CTATTTGCCCAGCCCCAGCCCTGGAGAGTCCTTTAGCAGGGCAAAGTGCAACATAGGCAGACCTTAAGGGATGACTCAGTAACAGATAAGCTTTGTGTGCCTGCAGGGCATATAAAACAGGGGCAAGGCACAGACTCATAGCAGA |
| | GCAATCACCACCAAGCCTGGAATAACTGCAGCCACC |
| 397 | > SP0268 (LVR_132_A1) |
| | AGGTTAATTTTTAAAAAGCAGTCAAAAGTCCAAGTGGCCCTTGGCAGCATTTACTCTCTCTGTTTGCTCTGGTTAATAATCTCAGGAGCACAAACATTCCTGTACCAGAATGAACATTGAACTTTGGACTATACCTGAGGGGTGA |
| | GGTAAACAACAGGACTATAAATGGCCCGGGAGGCGCCCTTTGGACCTTTTGCAATCCTGGCGCACTGAACCCTTGACCCCTGCCCTGCAGCCCCCGCAGCTTGCTGTTTGCCCACTCTATTTGCCCAGCCCCAGCCCTGGAGAGT |
| | CCTTTAGCAGGGCAAAGTGCAACATAGGCAGACCTTAAGGGATGACTCAGTAACAGATAAGCTTTGTGTGCCTGCAGGGCATATAAAACAGGGGCAAGGCACAGACTCATAGCAGAGCAATCACCACCAAGCCTGGAATAACTGC |
| | AGCCACC |
| 398 | > SP0269 (LVR_132_V1) |
| | AAGCAAATATTTGTGGTTATGGATTAACTCGAACTGTTTGCCCACTCTATTTGCCCTGTACCAGAATGAACATTGAACTTTGGACTATACCTGAGGGGTGAGGTAAACAACAGGACTATAAATGGCCCGGGAGGCGCCCTTTGGA |
| | CCTTTTGCAATCCTGGCGCACTGAACCCTTGACCCCTGCCCTGCAGCCCCCGCAGCTTGCTGTTTGCCCACTCTATTTGCCCAGCCCCAGCCCTGGAGAGTCCTTTAGCAGGGCAAAGTGCAACATAGGCAGACCTTAAGGGATG |
| | ACTCAGTAACAGATAAGCTTTGTGTGCCTGCAGGGCATATAAAACAGGGGCAAGGCACAGACTCATAGCAGAGCAATCACCACCAAGCCTGGAATAACTGCAGCCACC |
| 399 | > SP0270 (LVR 132_V2) |
| | GGCGCCCTTTGGACCTTTTGCAATCCTGGAGCAAACAGCAAACACTGTACCAGAATGAACATTGAACTTTGGACTATACCTGAGGGGTGAGGTAAACAACAGGACTATAAATGGCCCGGGAGGCGCCCTTTGGACCTTTTGCAAT |
| | CCTGGCGCACTGAACCCTTGACCCCTGCCCTGCAGCCCCCGCAGCTTGCTGTTTGCCCACTCTATTTGCCCAGCCCCAGCCCTGGAGAGTCCTTTAGCAGGGCAAAGTGCAACATAGGCAGACCTTAAGGGATGACTCAGTAACA |
| | GATAAGCTTTGTGTGCCTGCAGGGCATATAAAACAGGGGCAAGGCACAGACTCATAGCAGAGCAATCACCACCAAGCCTGGAATAACTGCAGCCACC |
| 400 | > SP0271 (LVR 133_A1) |
| | AGGTTAATTTTTAAAAAGCAGTCAAAAGTCCAAGTGGCCCTTGGCAGCATTTACTCTCTCTGTTTGCTCTGGTTAATAATCTCAGGAGCACAAACATTCCTGTACCAGAATGAACATTGAACTTTGGACTATACCTGAGGGGTGA |
| | GGTAAACAACAGGACTATAAATGGCCCGGGAGGCGCCCTTTGGACCTTTTGCAATCCTGGCGCTCTTTTGTTTTACATGAAGGGTCTGGCAGCCAAAGCAATCACTCAAAGTTCAAACCTTATCATTTTTTGCTTTGTTCCTCTT |
| | GGCCTTGGTTTTGTACATCAGCTTTGAAAATACCATCCCAGGGTTAATGCTGGGGTTAATTTATAACTAAGAGTGCTCTAGTTTTGCAATACAGGACATGCTATAAAAATGGAAAGATGTTGCTTTCTGAGAGATGCGCCACC |
| 401 | > SP0272 (LVR_133_V1) |
| | AAGCAAATATTTGTGGTTATGGATTAACTCGAACTGTTTGCCCACTCTATTTGCCCTGTACCAGAATGAACATTGAACTTTGGACTATACCTGAGGGGTGAGGTAAACAACAGGACTATAAATGGCCCGGGAGGCGCCCTTTGGA |
| | CCTTTTGCAATCCTGGCGCTCTTTTGTTTTACATGAAGGGTCTGGCAGCCAAAGCAATCACTCAAAGTTCAAACCTTATCATTTTTTGCTTTGTTCCTCTTGGCCTTGGTTTTGTACATCAGCTTTGAAAATACCATCCCAGGGT |
| | TAATGCTGGGGTTAATTTATAACTAAGAGTGCTCTAGTTTTGCAATACAGGACATGCTATAAAAATGGAAAGATGTTGCTTTCTGAGAGATGCGCCACC |
| 402 | > SP0273 (LVR_133_V2) |
| | GGCGCCCTTTGGACCTTTTGCAATCCTGGAGCAAACAGCAAACACTGTACCAGAATGAACATTGAACTTTGGACTATACCTGAGGGGTGAGGTAAACAACAGGACTATAAATGGCCCGGGAGGCGCCCTTTGGACCTTTTGCAAT |
| | CCTGGCGCTCTTTTGTTTTACATGAAGGGTCTGGCAGCCAAAGCAATCACTCAAAGTTCAAACCTTATCATTTTTTGCTTTGTTCCTCTTGGCCTTGGTTTTGTACATCAGCTTTGAAAATACCATCCCAGGGTTAATGCTGGGG |
| | TTAATTTATAACTAAGAGTGCTCTAGTTTTGCAATACAGGACATGCTATAAAAATGGAAAGATGTTGCTTTCTGAGAGATGCGCCACC |
| 403 | > SP0368 |
| | AGGTTAATTTTTAAAAAGCAGTCAAAAGTCCAAGTGGCCCTTGGCAGCATTTACTCTCTCTGTTTGCTCTGGTTAATAATCTCAGGAGCACAAACATTCCAGTCATATGTTTGCTCACTGAAGGTTACTAGTTAACAGGCATCCC |
| | TTAAACAGGATATAAAAGGACTTCAGCAGGACTGCTCGAAACATCCCACTCAGCCACC |
| 404 | > SP0373 |
| | AGGTTAATTTTTAAAAAGCAGTCAAAAGTCCAAGTGGCCCTTGGCAGCATTTACTCTCTCTGTTTGCTCTGGTTAATAATCTCAGGAGCACAAACATTCCTGTACCGGCCCGGGAGGCGCCCTTTGGACCTTTTGCAATCCTGGC |
| | GGGGCATATAAAACAGGGGCAAGGCACAGACTCATAGCAGAGCAATCACCACCAAGCCTGGAATAACTGCAGCCACC |
| 405 | > SP0378 |
| | TGCTCTCTGACAAAGATACGGTGGGTCCCACTGATGAACTGTGCTGCCACAGTAAATGTAGCCACTATGCCTATCTCCATTCTGAAGATGTGCCCTGTTCAAACATGTCCTAATACTCTGTCTCTGCAAGGGTCATCAGTAGTTT |
| | TCCATCTTACTCAACATCCTCCCAGTGAGTCATATGTTTGCTCACTGAAGGTTACTAGTTAACAGGCATCCCTTAAACAGGATATAAAAGGACTTCAGCAGGACTGCTCGAAACATCCCACTCAGCCACC |
| 406 | > SP0379 |
| | CCTCCCCGTGTTCCTGCTCTTTGTCCCTCTGTCCTACTTAGACTAATATTTGCCTTGGGTACTGCAAACAGGAAATGGGGGAGGGACAGGAGTAGGGCGGCCCTGTTCAAACATGTCCTAATACTCTGTCTCTGCAAGGGTCATC |
| | AGTAGTTTTCCATCTTACTCAACATCCTCCCAGTGAGTCATATGTTTGCTCACTGAAGGTTACTAGTTAACAGGCATCCCTTAAACAGGATATAAAAGGACTTCAGCAGGACTGCTCGAAACATCCCACTCAGCCACC |
| 407 | > SP0380 |
| | CCGCCCCCACTGAACCCTTGACCCCTGCCCTGCAGCCCCCGCAGCTTGCTGTTTGCCCACTCTATTTGCCCAGCCCCAGCCCTGTTCAAACATGTCCTAATACTCTGTCTCTGCAAGGGTCATCAGTAGTTTTCCATCTTACTCA |
| | ACATCCTCCCAGTGAGTCATATGTTTGCTCACTGAAGGTTACTAGTTAACAGGCATCCCTTAAACAGGATATAAAAGGACTTCAGCAGGACTGCTCGAAACATCCCACTCAGCCACC |
| 408 | > SP0381 |
| | AAGCTTTCTGAACAGCCAAACAGAGATTCCAAAGTTCAGGCACCAAAGTTCAGACCCTAACAGTTATTTACAAGGGTCAGTTAACCCCTGTTCAAACATGTCCTAATACTCTGTCTCTGCAAGGGTCATCAGTAGTTTTCCATCT |
| | TACTCAACATCCTCCCAGTGAGTCATATGTTTGCTCACTGAAGGTTACTAGTTAACAGGCATCCCTTAAACAGGATATAAAAGGACTTCAGCAGGACTGCTCGAAACATCCCACTCAGCCACC |
| 409 | > SP0272 (LVR_133_V1) |
| | AAGCAAATATTTGTGGTTATGGATTAACTCGAACTGTTTGCCCACTCTATTTGCCCTGTACCAGAATGAACATTGAACTTTGGACTATACCTGAGGGGTGAGGTAAACAACAGGACTATAAATGGCCCGGGAGGCGCCCTTTGGA |
| | CCTTTTGCAATCCTGGCGCTCTTTTGTTTTACATGAAGGGTCTGGCAGCCAAAGCAATCACTCAAAGTTCAAACCTTATCATTTTTTGCTTTGTTCCTCTTGGCCTTGGTTTTGTACATCAGCTTTGAAAATACCATCCCAGGGT |
| | TAATGCTGGGGTTAATTTATAACTAAGAGTGCTCTAGTTTTGCAATACAGGACATGCTATAAAAATGGAAAGATGTTGCTTTCTGAGAGATGCGCCACC |
| 410 | > SP0273 (LVR 133_V2) |
| | GGCGCCCTTTGGACCTTTTGCAATCCTGGAGCAAACAGCAAACACTGTACCAGAATGAACATTGAACTTTGGACTATACCTGAGGGGTGAGGTAAACAACAGGACTATAAATGGCCCGGGAGGCGCCCTTTGGACCTTTTGCAAT |
| | CCTGGCGCTCTTTTGTTTTACATGAAGGGTCTGGCAGCCAAAGCAATCACTCAAAGTTCAAACCTTATCATTTTTTGCTTTGTTCCTCTTGGCCTTGGTTTTGTACATCAGCTTTGAAAATACCATCCCAGGGTTAATGCTGGGG |
| | TTAATTTATAACTAAGAGTGCTCTAGTTTTGCAATACAGGACATGCTATAAAAATGGAAAGATGTTGCTTTCTGAGAGATGCGCCACC |
| 411 | > SP0368 |
| | AGGTTAATTTTTAAAAAGCAGTCAAAAGTCCAAGTGGCCCTTGGCAGCATTTACTCTCTCTGTTTGCTCTGGTTAATAATCTCAGGAGCACAAACATTCCAGTCATATGTTTGCTCACTGAAGGTTACTAGTTAACAGGCATCCC |
| | TTAAACAGGATATAAAAGGACTTCAGCAGGACTGCTCGAAACATCCCACTCAGCCACC |
| 412 | > SP0373 |
| | AGGTTAATTTTTAAAAAGCAGTCAAAAGTCCAAGTGGCCCTTGGCAGCATTTACTCTCTCTGTTTGCTCTGGTTAATAATCTCAGGAGCACAAACATTCCTGTACCGGCCCGGGAGGCGCCCTTTGGACCTTTTGCAATCCTGGC |
| | GGGGCATATAAAACAGGGGCAAGGCACAGACTCATAGCAGAGCAATCACCACCAAGCCTGGAATAACTGCAGCCACC |
| 413 | > SP0378 |
| | TGCTCTCTGACAAAGATACGGTGGGTCCCACTGATGAACTGTGCTGCCACAGTAAATGTAGCCACTATGCCTATCTCCATTCTGAAGATGTGCCCTGTTCAAACATGTCCTAATACTCTGTCTCTGCAAGGGTCATCAGTAGTTT |
| | TCCATCTTACTCAACATCCTCCCAGTGAGTCATATGTTTGCTCACTGAAGGTTACTAGTTAACAGGCATCCCTTAAACAGGATATAAAAGGACTTCAGCAGGACTGCTCGAAACATCCCACTCAGCCACC |
| 414 | > SP0379 |
| | CCTCCCCGTGTTCCTGCTCTTTGTCCCTCTGTCCTACTTAGACTAATATTTGCCTTGGGTACTGCAAACAGGAAATGGGGGAGGGACAGGAGTAGGGCGGCCCTGTTCAAACATGTCCTAATACTCTGTCTCTGCAAGGGTCATC |
| | AGTAGTTTTCCATCTTACTCAACATCCTCCCAGTGAGTCATATGTTTGCTCACTGAAGGTTACTAGTTAACAGGCATCCCTTAAACAGGATATAAAAGGACTTCAGCAGGACTGCTCGAAACATCCCACTCAGCCACC |
| 415 | > SP0380 |
| | CCGCCCCCACTGAACCCTTGACCCCTGCCCTGCAGCCCCCGCAGCTTGCTGTTTGCCCACTCTATTTGCCCAGCCCCAGCCCTGTTCAAACATGTCCTAATACTCTGTCTCTGCAAGGGTCATCAGTAGTTTTCCATCTTACTCA |
| | ACATCCTCCCAGTGAGTCATATGTTTGCTCACTGAAGGTTACTAGTTAACAGGCATCCCTTAAACAGGATATAAAAGGACTTCAGCAGGACTGCTCGAAACATCCCACTCAGCCACC |
| 416 | > SP0381 |
| | AAGCTTTCTGAACAGCCAAACAGAGATTCCAAAGTTCAGGCACCAAAGTTCAGACCCTAACAGTTATTTACAAGGGTCAGTTAACCCCTGTTCAAACATGTCCTAATACTCTGTCTCTGCAAGGGTCATCAGTAGTTTTCCATCT |
| | TACTCAACATCCTCCCAGTGAGTCATATGTTTGCTCACTGAAGGTTACTAGTTAACAGGCATCCCTTAAACAGGATATAAAAGGACTTCAGCAGGACTGCTCGAAACATCCCACTCAGCCACC |
| 417 | > SP0384 |
| | AGGTTAATTTTTAAAAAGCAGTCAAAAGTCCAAGTGGCCCTTGGCAGCATTTACTCTCTCTGTTTGCTCTGGTTAATAATCTCAGGAGCACAAACATTCCGGCCCGGGAGGCGCCCTTTGGACCTTTTGCAATCCTGGCGCCGAT |
| | GACCTAATGATTCTGAGCTTGGCAAAGGTCTTATCTCCCAGCTCGCCCAGGCCCAGTGTTCCAGGAATGTGACCTTTGCTGCAGCAGCCGCTGGAGGGGGCAGAGGGGATGGGCTGGAGGTTGAGCAAACAGAGCAGCAGAAAAG |
| | GCAGTTCCTCTTCTCCAGTGCCCTCCTTCCCTGTCTCTGCCTCTCCCTCCCTTCCTCAGGCATCAGAGCGGAGACTTCAGGGAGACCAGAGCCCAGCTTGCCAGGCACTGAGCTAGAAGCCCTGCCGCCACC |
| 418 | > SP0388 |
| | AGGTTAATTTTTAAAAAGCAGTCAAAAGTCCAAGTGGCCCTTGGCAGCATTTACTCTCTCTGTTTGCTCTGGTTAATAATCTCAGGAGCACAAACATTCCGGGCATATAAAACAGGGGCAAGGCACAGACTCATAGCAGAGCAAT |
| | CACCACCAAGCCTGGAATAACTGCAGCCACC |
| 419 | > SP0396 |
| | AGGTTAATTTTTAAAAAGCAGTCAAAAGTCCAAGTGGCCCTTGGCAGCATTTACTCTCTCTGTTTGCTCTGGTTAATAATCTCAGGAGCACAAACATTCCTGTACCGGCCCGGGAGGCGCCCTTTGGACCTTTTGCAATCCTGGC |
| | GCCCTGGAGAGTCCTTTAGCAGGGCAAAGTGCAACATAGGCAGACCTTAAGGGATGACTCAGTAACAGATAAGCTTTGTGTGCCTGCAGGGCATATAAAACAGGGGCAAGGCACAGACTCATAGCAGAGCAATCACCACCAAGCC |
| | TGGAATAACTGCAGCCACC |
| 420 | > SP0397 |
| | AGGTTAATTTTTAAAAAGCAGTCAAAAGTCCAAGTGGCCCTTGGCAGCATTTACTCTCTCTGTTTGCTCTGGTTAATAATCTCAGGAGCACAAACATTCCTGTACCCACTGAACCCTTGACCCCTGCCCTGCAGCCCCCGCAGCT |
| | TGCTGTTTGCCCACTCTATTTGCCCAGCCCCAGCCCTGGAGAGTCCTTTAGCAGGGCAAAGTGCAACATAGGCAGACCTTAAGGGATGACTCAGTAACAGATAAGCTTTGTGTGCCTGCAGGGCATATAAAACAGGGGCAAGGCA |
| | CAGACTCATAGCAGAGCAATCACCACCAAGCCTGGAATAACTGCAGCCACC |
| 421 | > SP0398 |
| | CAGGCTTTCACTTTCTCGCCAACTTACAAGGCCTTTCTGTGTAAACAATACCTGAACCTTTACCCCGTTGCCCGGCAACGGCCAGGTCTGTGCCAAGTGTTTGAGGTTAATTTTTAAAAAGCAGTCAAAAGTCCAAGTGGCCCTT |
| | GGCAGCATTTACTCTCTCTGTTTGCTCTGGTTAATAATCTCAGGAGCACAAACATTCCTGTACCAGAATGAACATTGAACTTTGGACTATACCTGAGGGGTGAGGTAAACAACAGGACTATAAATGGCCCGGGAGGCGCCCTTTG |
| | GACCTTTTGCAATCCTGGCGCACTGAACCCTTGACCCCTGCCCTGCAGCCCCCGCAGCTTGCTGTTTGCCCACTCTATTTGCCCAGCCCCAGCCCTGGAGAGTCCTTTAGCAGGGCAAAGTGCAACATAGGCAGACCTTAAGGGA |
| | TGACTCAGTAACAGATAAGCTTTGTGTGCCTGCAGGGCATATAAAACAGGGGCAAGGCACAGACTCATAGCAGAGCAATCACCACCAAGCCTGGAATAACTGCAGCCACC (SEQ ID NO: 249) |
| 422 | > SP0399 |
| | AGAATGAACATTGAACTTTGGACTATACCTGAGGGGTGAGGTAAACAACAGGACTATAAATGGGCATATAAAACAGGGGCAAGGCACAGACTCATAGCAGAGCAATCACCACCAAGCCTGGAATAACTGCAGCCACC |
| 423 | > SP0403 |
| | CAGGCTTTCACTTTCTCGCCAACTTACAAGGCCTTTCTGTGTAAACAATACCTGAACCTTTACCCCGTTGCCCGGCAACGGCCAGGTCTGTGCCAAGTGTTTGAAGCAAATATTTGTGGTTATGGATTAACTCGAACTGTTTGCC |
| | CACTCTATTTGCCCTGTACCGGCCCGGGAGGCGCCCTTTGGACCTTTTGCAATCCTGGCGCACTGAACCCTTGACCCCTGCCCTGCAGCCCCCGCAGCTTGCTGTTTGCCCACTCTATTTGCCCAGCCCCAGCCCTGGAGAGTCC |
| | TTTAGCAGGGCAAAGTGCAACATAGGCAGACCTTAAGGGATGACTCAGTAACAGATAAGCTTTGTGTGCCTGCAGGGCATATAAAACAGGGGCAAGGCACAGACTCATAGCAGAGCAATCACCACCAAGCCTGGAATAACTGCAG |
| | CCACC |
| 424 | > SP0404 |
| | CAGGCTTTCACTTTCTCGCCAACTTACAAGGCCTTTCTGTGTAAACAATACCTGAACCTTTACCCCGTTGCCCGGCAACGGCCAGGTCTGTGCCAAGTGTTTGAAGCAAATATTTGTGGTTATGGATTAACTCGAACTGTTTGCC |
| | CACTCTATTTGCCCTGTACCAGAATGAACATTGAACTTTGGACTATACCTGAGGGGTGAGGTAAACAACAGGACTATAAATGGCCCGGGAGGCGCCCTTTGGACCTTTTGCAATCCTGGCGCACTGAACCCTTGACCCCTGCCCT |
| | GCAGCCCCCGCAGCTTGCTGTTTGCCCACTCTATTTGCCCAGCCCCAGCCCTGGAGAGTCCTTTAGCAGGGCAAAGTGCAACATAGGCAGACCTTAAGGGATGACTCAGTAACAGATAAGCTTTGTGTGCCTGCAGGGCATATAA |
| | AACAGGGGCAAGGCACAGACTCATAGCAGAGCAATCACCACCAAGCCTGGAATAACTGCAGCCACC |
| 425 | > SP0405 |
| | CAGGCTTTCACTTTCTCGCCAACTTACAAGGCCTTTCTGTGTAAACAATACCTGAACCTTTACCCCGTTGCCCGGCAACGGCCAGGTCTGTGCCAAGTGTTTGAAGCAAATATTTGTGGTTATGGATTAACTCGAACTGTTTGCC |
| | CACTCTATTTGCCCTGTACCAGAATGAACATTGAACTTTGGACTATACCTGAGGGGTGAGGTAAACAACAGGACTATAAATGGCCCGGGAGGCGCCCTTTGGACCTTTTGCAATCCTGGCGCACTGAACCCTTGACCCCTGCCCT |
| | GCAGCCCCCGCAGCTTGCTGTTTGCCCACTCTATTTGCCCAGCCCCAGGGGCATATAAAACAGGGGCAAGGCACAGACTCATAGCAGAGCAATCACCACCAAGCCTGGAATAACTGCAGCCACC |
| 426 | >SP0406 |
| | AGGTTAATTTTTAAAAAGCAGTCAAAAGTCCAAGTGGCCCTTGGCAGCATTTACTCTCTCTGTTTGCTCTGGTTAATAATCTCAGGAGCACAAACATTCCGGCCCGGGAGGCGCCCTTTGGACCTTTTGCAATCCTGGCGAGTCA |
| | TATGTTTGCTCACTGAAGGTTACTAGTTAACAGGCATCCCTTAAACAGGATATAAAAGGACTTCAGCAGGACTGCTCGAAACATCCCACTCAGCCACC |
| 427 | > SP0407 |
| | AGGTTAATTTTTAAAAAGCAGTCAAAAGTCCAAGTGGCCCTTGGCAGCATTTACTCTCTCTGTTTGCTCTGGTTAATAATCTCAGGAGCACAAACATTCCCAGGCTTTCACTTTCTCGCCAACTTACAAGGCCTTTCTGTGTAAA |
| | CAATACCTGAACCTTTACCCCGTTGCCCGGCAACGGCCAGGTCTGTGCCAAGTGTTTGGGGCATATAAAACAGGGGCAAGGCACAGACTCATAGCAGAGCAATCACCACCAAGCCTGGAATAACTGCAGCCACC |
| 428 | > SP0409 |
| | AGGTTAATTTTTAAAAAGCAGTCAAAAGTCCAAGTGGCCCTTGGCAGCATTTACTCTCTCTGTTTGCTCTGGTTAATAATCTCAGGAGCACAAACATTCCCCCTGTTCAAACATGTCCTAATACTCTGTCTCTGCAAGGGTCATC |
| | AGTAGTTTTCCATCTTACTCAACATCCTCCCAGTGGGGCATATAAAACAGGGGCAAGGCACAGACTCATAGCAGAGCAATCACCACCAAGCCTGGAATAACTGCAGCCACC |
| 429 | > SP0411 |
| | CCCTGTTCAAACATGTCCTAATACTCTGTCTCTGCAAGGGTCATCAGTAGTTTTCCATCTTACTCAACATCCTCCCAGTGAGTCATATGTTTGCTCACTGAAGGTTACTAGTTAACAGGCATCCCTTAAACAGGATATAAAAGGA |
| | CTTCAGCAGGACTGCTCGAAACATCCCACTCAGCCACC |
| 91 | > SP0412 |
| | AGGTTAATTTTTAAAAAGCAGTCAAAAGTCCAAGTGGCCCTTGGCAGCATTTACTCTCTCTGTTTGCTCTGGTTAATAATCTCAGGAGCACAAACATTCCCCCTGTTCAAACATGTCCTAATACTCTGTCTCTGCAAGGGTCATC |
| | AGTAGTTTTCCATCTTACTCAACATCCTCCCAGTGAGTCATATGTTTGCTCACTGAAGGTTACTAGTTAACAGGCATCCCTTAAACAGGATATAAAAGGACTTCAGCAGGACTGCTCGAAACATCCCACTCAGCCACC |
| 430 | > SP0413 |
| | AGGTTAATTTTTAAAAAGCAGTCAAAAGTCCAAGTGGCCCTTGGCAGCATTTACTCTCTCTGTTTGCTCTGGTTAATAATCTCAGGAGCACAAACATTCCTGTACCGGCCCGGGAGGCGCCCTTTGGACCTTTTGCAATCCTGGC |
| | GAGTCATATGTTTGCTCACTGAAGGTTACTAGTTAACAGGCATCCCTTAAACAGGATATAAAAGGACTTCAGCAGGACTGCTCGAAACATCCCACTCAGCCACC |
| 431 | >TTR promoter |
| | TCGAGCTTGGGCTGCAGGTCGAGGGCACTGGGAGGATGTTGAGTAAGATGGAAAACTACTGATGACCCTTGCAGAGACAGAGTATTAGGACATGTTTGAACAGGGGCCGGGCGATCAGCAGGTAGCTCTAGAGGATCCCCGTCTG |
| | TCTGCACATTTCGTAGAGCGAGTGTTCCGATACTCTAATCTCCCTAGGCAAGGTTCATATTTGTGTAGGTTACTTATTCTCCTTTTGTTGACTAAGTCAATAATCAGAATCAGCAGGTTTGGAGTCAGCTTGGCAGGGATCAGCA |
| | GCCTGGGTTGGAAGGAGGGGGTATAAAAGCCCCTTCACCAGGAGAAGCCGTCACACAGA |
| 432 | > LP1 |
| | CCCTAAAATGGGCAAACATTGCAAGCAGCAAACAGCAAACACACAGCCCTCCCTGCCTGCTGACCTTGGAGCTGGGGCAGAGGTCAGAGACCTCTCTGGGCCCATGCCACCTCCAACATCCACTCGACCCCTTGGAATTTCGGTG |
| | GAGAGGAGCAGAGGTTGTCCTGGCGTGGTTTAGGTAGTGTGAGAGGGGAATGACTCCTTTCGGTAAGTGCAGTGGAAGCTGTACACTGCCCAGGCAAAGCGTCCGGGCAGCGTAGGCGGGCGACTCAGATCCCAGCCAGTGGACT |
| | TAGCCCCTGTTTGCTCCTCCGATAACTGGGGTGACCTTGGTTAATATTCACCAGCAGCCTCCCCCGTTGCCCCTCTGGATCCACTGCTTAAATACGGACGAGGACAGGGCCCTGTCTCCTCAGCTTCAGGCACCACCACTGACCT |
| | GGGACAGTGAATCCGGACTCTAAGGTAAATATAAAATTTTTAAGTGTATAATGTGTTAAACTACTGATTCTAATTGTTTCTCTCTTTTAGATTCCAACCTTTGGAACTGAATTCTAGACCACC |
| 433 | > CMV-IE |
| | ATAGTAATCAATTACGGGGTCATTAGTTCATAGCCCATATATGGAGTTCCGCGTTACATAACTTACGGTAAATGGCCCGCCTGGCTGACCGCCCAACGACCCCCGCCCATTGACGTCAATAATGACGTATGTTCCCATAGTAACG |
| | CCAATAGGGACTTTCCATTGACGTCAATGGGTGGAGTATTTACGGTAAACTGCCCACTTGGCAGTACATCAAGTGTATCATATGCCAAGTACGCCCCCTATTGACGTCAATGACGGTAAATGGCCCGCCTGGCATTATGCCCAGT |
| | ACATGACCTTATGGGACTTTCCTACTTGGCAGTACATCTACGTATTAGTCATCGCTATTACCATGGTGATGCGGTTTTGGCAGTACATCAATGGGCGTGGATAGCGGTTTGACTCACGGGGATTTCCAAGTCTCCACCCCATTGA |
| | CGTCAATGGGAGTTTGTTTTGGCACCAAAATCAACGGGACTTTCCAAAATGTCGTAACAACTCCGCCCCATTGACGCAAATGGGCGGTAGGCGTGTACGGTGGGAGGTCTATATAAGCAGAGCTGGTTTAGTGAACCGTCAGATC |
| 434 | > CBA |
| | AGATCTGAATTCGGTACCTAGTTATTAATAGTAATCAATTACGGGGTCATTAGTTCATAGCCCATATATGGAGTTCCGCGTTACATAACTTACGGTAAATGGCCCGCCTGGCTGACCGCCCAACGACCCCCGCCCATTGACGTCA |
| | ATAATGACGTATGTTCCCATAGTAACGCCAATAGGGACTTTCCATTGACGTCAATGGGTGGAGTATTTACGGTAAACTGCCCACTTGGCAGTACATCAAGTGTATCATATGCCAAGTACGCCCCCTATTGACGTCAATGACGGTA |
| | AATGGCCCGCCTGGCATTATGCCCAGTACATGACCTTATGGGACTTTCCTACTTGGCAGTACATCTACGTATTAGTCATCGCTATTACCATGGTCGAGGTGAGCCCCACGTTCTGCTTCACTCTCCCCATCTCCCCCCCCTCCCC |
| | ACCCCCAATTTTGTATTTATTTATTTTTTAATTATTTTGTGCAGCGATGGGGGCGGGGGGGGGGGGGGGGCGCGCGCCAGGCGGGGCGGGGCGGGGCGAGGGGCGGGGCGGGGCGAGGCGGAGAGGTGCGGCGGCAGCCAATCAG |
| | AGCGGCGCGCTCCGAAAGTTTCCTTTTATGGCGAGGCGGCGGCGGCGGCGGCCCTATAAAAAGCGAAGCGCGCGGCGGGCGGGAGTCGCTGCGCGCTGCCTTCGCCCCGTGCCCCGCTCCGCCGCCGCCTCGCGCCGCCCGCCCC |
| | GGCTCTGACTGACCGCGTTACTCCCACAGGTGAGCGGGCGGGACGGCCCTTCTCCTCCGGGCTGTAATTAGCGCTTGGTTTAATGACGGCTTGTTTCTTTTCTGTGGCTGCGTGAAAGCCTTGAGGGGCTCCGGGAGGGCCCTTT |
| | GTGCGGGGGGAGCGGCTCGGGGGGTGCGTGCGTGTGTGTGTGCGTGGGGAGCGCCGCGTGCGGCTCCGCGCTGCCCGGCGGCTGTGAGCGCTGCGGGCGCGGCGCGGGGCTTTGTGCGCTCCGCAGTGTGCGCGAGGGGAGCGCG |
| | GCCGGGGGCGGTGCCCCGCGGTGCGGGGGGGGCTGCGAGGGGAACAAAGGCTGCGTGCGGGGTGTGTGCGTGGGGGGGTGAGCAGGGGGTGTGGGCGCGTCGGTCGGGCTGCAACCCCCCCTGCACCCCCCTCCCCGAGTTGCTG |
| | AGCACGGCCCGGCTTCGGGTGCGGGGCTCCGTACGGGGCGTGGCGCGGGGCTCGCCGTGCCGGGCGGGGGGTGGCGGCAGGTGGGGGTGCCGGGCGGGGGGGGGCCGCCTCGGGCCGGGGAGGGCTCGGGGGAGGGGCGCGGCGG |
| | CCCCCGGAGCGCCGGCGGCTGTCGAGGCGCGGCGAGCCGCAGCCATTGCCTTTTATGGTAATCGTGCGAGAGGGCGCAGGGACTTCCTTTGTCCCAAATCTGTGCGGAGCCGAAATCTGGGAGGCGCCGCCGCACCCCCTCTAGC |
| | GGGCGCGGGGCGAAGCGGTGCGGCGCCGGCAGGAAGGAAATGGGCGGGGAGGGCCTTCGTGCGTCGCCGCGCCGCCGTCCCCTTCTCCCTCTCCAGCCTCGGGGCTGTCCGCGGGGGGACGGCTGCCTTCGGGGGGGACGGGGCA |
| | GGGCGGGGTTCGGCTTCTGGCGTGTGACCGGCGGCTCTAGAGCCTCTGCTAACCATGTTCATGCCTTCTTCTTTTTCCTACAGCTCCTGGGCAACGTGCTGGTTATTGTGCTGTCTCATCATTTTGGCAAAGAATTCCTCGAAGA |
| | TCTAGGCAACGCGTCTCGAGGCGGCCGCCGCCACC |
| 435 | > TBG |
| | AGGTTAATTTTTAAAAAGCAGTCAAAAGTCCAAGTGGCCCTTGGCAGCATTTACTCTCTCTGTTTGCTCTGGTTAATAATCTCAGGAGCACAAACATTCCAGATCCAGGTTAATTTTTAAAAAGCAGTCAAAAGTCCAAGTGGCC |
| | CTTGGCAGCATTTACTCTCTCTGTTTGCTCTGGTTAATAATCTCAGGAGCACAAACATTCCAGATCCGGCGCGCCAGGGCTGGAAGCTACCTTTGACATCATTTCCTCTGCGAATGCATGTATAATTTCTACAGAACCTATTAGA |
| | AAGGATCACCCAGCCTCTGCTTTTGTACAACTTTCCCTTAAAAAACTGCCAATTCCACTGCTGTTTGGCCCAATAGTGAGAACTTTTTCCTGCTGCCTCTTGGTGCTTTTGCCTATGGCCCCTATTCTGCCTGCTGAAGACACTC |
| | TTGCCAGCATGGACTTAAACCCCTCCAGCTCTGACAATCCTCTTTCTCTTTTGTTTTACATGAAGGGTCTGGCAGCCAAAGCAATCACTCAAAGTTCAAACCTTATCATTTTTTGCTTTGTTCCTCTTGGCCTTGGTTTTGTACA |
| | TCAGCTTTGAAAATACCATCCCAGGGTTAATGCTGGGGTTAATTTATAACTAAGAGTGCTCTAGTTTTGCAATACAGGACATGCTATAAAAATGGAAAGATGTTGCTTTCTGAGAGACTGCAGAAGTTGGTCGTGAGGCACTGGG |
| | CAGGTAAGTATCAAGGTTACAAGACAGGTTTAAGGAGACCAATAGAAACTGGGCTTGTCGAGACAGAGAAGACTCTTGCGTTTCTGATAGGCACCTATTGGTCTTACTGACATCCACTTTGCCTTTCTCTCCACAGGGCAATCCG |
| | GTACTGTTGGTAAAGCCACC |
| 438 | >CMV-IE 5′ UTR with 3′ Kozac GCCACC. |
| | TCAGATCGCCTGGAGACGCCATCCACGCTGTTTTGACCTCCATAGAAGACACCGGGACCGATCCAGCCTCCGCGGCCGGGAACGGTGCATTGGAACGCGGATTCCCCGTGCCAAGAGTGACGTAAGTACCGCCTATAGACTCTAT |
| | AGGCACACCCCTTTGGCTCTTATGCATGAACGGTGGAGGGCAGTGTAGTCTGAGCAGTACTCGTTGCTGCCGCGCGCGCCACCAGACATAATAGCTGACAGACTAACAGACTGTTCCTTTCCATGGGTCTTTTCTGCAGGCCACC |
| 439 | > LVR 0243-5′ UTR |
| | TGA AATGCCTGCC ATATATTAGT GCCCTGAAGT CCAAAGGTAG AGGAACCGAG TGTTTAAAAA TTACTGTGGC TGTGGAGTCA ACATGATGTA AAAAAACAAA CATTTGGATA ACACCAAGAA |
| | GCCAGATATG GTTGAAATGT TGACTGGTTG ACAAAAATAA TTTGGGTTGC TTAATGGTGC ACAAAGGTAA TGCAAAAAGG TTAATTTTTA AAAAGCAGTC AAAAGTCCAA GTGGCCCTTG GCAGCATTTA |
| | CTCTCTCTGT TTGCTCTGGT TAATAATCTC AGGAGCACAA ACATTCCGGC CCGGGAGGCG CCCTTTGGAC CTTTTGCAAT CCTGGCGCAC TGAACCCTTG ACCCCTGCCC TGCAGCCCCC GCAGCTTGCT |
| | GTTTGCCCAC TCTATTTGCC CAGCCCCAGC CCTGGAGAGT CCTTTAGCAG GGCAAAGTGC AACATAGGCA GACCTTAAGG GATGACTCAG TAACAGATAA GCTTTGTGTG CCTGCAGGGC ATATAAAACA |
| | GGGGCAAGGC ACAGACTCAT AGCAGAGCAA T |
| 440 | > LVR 0412-5′ UTR |
| | AGG TTAATTTTTA AAAAGCAGTC AAAAGTCCAA GTGGCCCTTG GCAGCATTTA CTCTCTCTGT TTGCTCTGGT TAATAATCTC AGGAGCACAA ACATTCCCCC TGTTCAAACA TGTCCTAATA |
| | CTCTGTCTCT GCAAGGGTCA TCAGTAGTTT TCCATCTTAC TCAACATCCT CCCAGTGAGT CATATGTTTG CTCACTGAAG GTTACTAGTT AACAGGCATC CCTTAAACAG GATATAAAAG GACTTCAGCA |
| | GGACTGCTCG AAACATCCCA CT |
| 441 | > AQ promoter (LVR_SP0131 A1) -5′ UTRs |
| | AGG TTAATTTTTA AAAAGCAGTC AAAAGTCCAA GTGGCCCTTG GCAGCATTTA CTCTCTCTGT TTGCTCTGGT TAATAATCTC AGGAGCACAA ACATTCCTGT ACCGGCCCGG GAGGCGCCCT |
| | TTGGACCTTT TGCAATCCTG GCGCACTGAA CCCTTGACCC CTGCCCTGCA GCCCCCGCAG CTTGCTGTTT GCCCACTCTA TTTGCCCAGC CCCAGCCCTG GAGAGTCCTT TAGCAGGGCA AAGTGCAACA |
| | TAGGCAGACC TTAAGGGATG ACTCAGTAAC AGATAAGCTT TGTGTGCCTG CAGGGCATAT AAAACAGGGG CAAGGCACAG ACTCATAGCA GAGCAAT |
| 442 | > MVM Intron |
| | CTAGCCCTAAGGTAAGTTGGCGCCGTTTAAGGGATGGTTGGTTGGTGGGGTATTAATGTTTAATTACCTTTTTTACAGGCCTGAA |
| 443 | > HBB2 intron sequence |
| | GTACACATAT TGACCAAATC AGGGTAATTT TGCATTTGTA ATTTTAAAAA ATGCTTTCTT CTTTTAATAT ACTTTTTTGT TTATCTTATT TCTAATACTT TCCCTAATCT CTTTCTTTCA GGGCAATAAT |
| | GATACAATGT ATCATGCCTC TTTGCACCAT TCTAAAGAAT AACAGTGATA ATTTCTGGGT TAAGGCAATA GCAATATTTC TGCATATAAA TATTTCTGCA TATAAATTGT AACTGATGTA AGAGGTTTCA |
| | TATTGCTAAT AGCAGCTACA ATCCAGCTAC CATTCTGCTT TTATTTTCTG GTTGGGATAA GGCTGGATTA TTCTGAGTCC AAGCTAGGCC CTTTTGCTAA TCTTGTTCAT ACCTCTTATC TTCCTCCCAC |
| | AGCTCCTGGG CAACCTGCTG GTCTCTCTGC TGGCCCATCA CTTTGGCAAA |
| | G |
| 444 | >HBB INTRON |
| | TACTAGCAGCTACAATCCAGCTACCATTCTGCTTTTATTTTATGGTTGGGATAAGGCTGGATTATTCTGAGTCCAAGCTAGGCCCTTTTGCTAATCATGTTCATACCTCTTATCTTCCTCCCACAGCTCCTGGGCAACGTGCTGG |
| | TCTGTGTGCTGGCCCATCACTTTGGCAAAGAATT |
| 445 | >UBC INTRON |
| | ACTTGGTGAGTAGCGGGCTGCTGGGCTGGCCGGGGCTTTCGTGGCCGCCGGGCCGCTCGGTGGGACGGAAGCGTGTGGAGAGACCGCCAAGGGCTGTAGTCTGGGTCCGCGAGCAAGGTTGCCCTGAACTGGGGGTTGGGGGGAG |
| | CGCAGCAAAATGGCGGCTGTTCCCGAGTCTTGAATGGAAGACGCTTGTGAGGCGGGCTGTGAGGTCGTTGAAACAAGGTGGGGGGCATGGTGGGCGGCAAGAACCCAAGGTCTTGAGGCCTTCGCTAATGCGGGAAAGCTCTTAT |
| | TCGGGTGAGATGGGCTGGGGCACCATCTGGGGACCCTGACGTGAAGTTTGTCACTGACTGGAGAACTCGGTTTGTCGTCTGTTGCGGGGGCGGCAGTTATGCGGTGCCGTTGGGCAGTGCACCCGTACCTTTGGGAGCGCGCGCC |
| | CTCGTCGTGTCGTGACGTCACCCGTTCTGTTGGCTTATAATGCAGGGTGGGGCCACCTGCCGGTAGGTGTGCGGTAGGCTTTTCTCCGTCGCAGGACGCAGGGTTCGGGCCTAGGGTAGGCTCTCCTGAATCGACAGGCGCCGGA |
| | CCTCTGGTGAGGGGAGGGATAAGTGAGGCGTCAGTTTCTTTGGTCGGTTTTATGTACCTATCTTCTTAAGTAGCTGAAGCTCCGGTTTTGAACTATGCGCTCGGGGTTGGCGAGTGTGTTTTGTGAAGTTTTTTAGGCACCTTTT |
| | GAAATGTAATCATTTGGGTCAATATGTAATTTTCAGTGTTAGACTAGTAAATTGTCCGCTAAATTCTGGCCGTTTTTGGCTTTTTTGTTAGAC |
| 446 | >MODIFIED CMVIE INTRON SEQUENCE. |
| | CAAGAGTGACGTAAGTACCGCCTATAGACTCTATAGGCACACCCCTTTGGCTCTTATGCATGAACGGTGGAGGGCAGTGTAGTCTGAGCAGTACTCGTTGCTGCCGCGCGCGCCACCAGACATAATAGCTGACAGACTAACAGAC |
| | TGTTCCTTTCCATGGGTCTTTTCTGCAG |
| 465 | >termination sequence |
| | TGATAACTCGAG |
| 464 | > pAAV-LSPhGAA w/mod endogenous GAA signal peptide |
| | CTGCGCGCTCGCTCGCTCACTGAGGCCGCCCGGGCAAAGCCCGGGCGTCGGGCGACCTTTGGTCGCCCGGCCTCAGTGAGCGAGCGAGCGCGCAGAGAGGGAGTGGCCAACTCCATCACTAGGGGTTCCTGGAGGGGTGGAGTCG |
| | TGACGTGAATTACGTCATAGGGTTAGGGAGGTCGGCCGCTCTAGGAGTTAATTTTTAAAAAGCAGTCAAAAGTCCAAGTGCCCTTGCGAGCATTTACTCTCTCTGTTTGCTCTGGTTAATAATCTCAGGAGCACAAACATTCCTT |
| | ACTAGTTCTAGGAGTTAATTTTTAAAAAGCAGTCAAAAGTCCAAGTGCCCTTGCGAGCATTTACTCTCTCTGTTTGCTCTGGTTAATAATCTCAGGAGCACAAACATTCCTTACTAGTTCTAGAGCGGCCGCCAGTGTGCTGGAA |
| | TTCGGCTTTTTTAGGGCTGGAAGCTACCTTTGACATCATTTCCTCTGCGAATGCATGTATAATTTCTACAGAACCTATTAGAAAGGATCACCCAGCCTCTGCTTTTGTACAACTTTCCCTTAAAAAACTGCCAATCCCACTGCTG |
| | TTTGGCCCAATAGTGAGAACTTTTTCCTGCTGCCTCTTGGTGCTTTTGCCTATGGCCCCTATTCTGCCTGCTGAAGACACTCTTGCCAGCATGGACTTAAACCCCTCCAGCTCTGACAATCCTCTTTCTCTTTTGTTTTACATGA |
| | AGGGTCTGGCAGCCAAAGCAATCACTCAAAGTTCAAACCTTATCATTTTTTGCTTTGTTCCTCTTGGCCTTGGTTTTGTACATCAGCTTTGAAAATACCATCCCAGGGTTAATGCTGGGGTTAATTTATAACTGAGAGTGCTCTA |
| | GTTCTGCAATACAGGACATGCTATAAAAATGGAAAGATGTTGCTTTCTGAGAGATCAGCTTACATGTGCTAGCGTAGCCAACCATGGGAGTGAGGCACCCGCCCTGCTCCCACCGGCTCCTGGCCGTCTGCGCCCTCGTGTCCTT |
| | GGCAACCGCTGCACTCCTGGGGCACATCCTACTCCATGATTTCCTGCTGGTTCCCCGAGAGCTGAGTGGCTCCTCCCCAGTCCTGGAGGAGACTCACCCAGCTCACCAGCAGGGAGCCAGCAGACCAGGGCCCCGGGACGCCCAG |
| | GCACACCCCGGCCGTCCCAGAGCAGTGCCCACACAGTGCGACGTCCCCCCCAACAGCCGCTTCGATTGCGCCCCTGACAAGGCCATCACCCAGGAACAGTGCGAGGCCCGCGGCTGCTGCTACATCCCTGCAAAGCAGGGGCTGC |
| | AGGGAGCCCAGATGGGGCAGCCCTGGTGCTTCTTCCCACCCAGCTACCCCAGCTACAAGCTGGAGAACCTGAGCTCCTCTGAAATGGGCTACACGGCCACCCTGACCCGTACCACCCCCACCTTCTTCCCCAAGGACATCCTGAC |
| | CCTGCGGCTGGACGTGATGATGGAGACTGAGAACCGCCTCCACTTCACGATCAAAGATCCAGCTAACAGGCGCTACGAGGTGCCCTTGGAGACCCCGCGTGTCCACAGCCGGGCACCGTCCCCACTCTACAGCGTGGAGTTCTCC |
| | GAGGAGCCCTTCGGGGTGATCGTGCACCGGCAGCTGGACGGCCGCGTGCTGCTGAACACGACGGTGGCGCCCCTGTTCTTTGCGGACCAGTTCCTTCAGCTGTCCACCTCGCTGCCCTCGCAGTATATCACAGGCCTCGCCGAGC |
| | ACCTCAGTCCCCTGATGCTCAGCACCAGCTGGACCAGGATCACCCTGTGGAACCGGGACCTTGCGCCCACGCCCGGTGCGAACCTCTACGGGTCTCACCCTTTCTACCTGGCGCTGGAGGACGGCGGGTCGGCACACGGGGTGTT |
| | CCTGCTAAACAGCAACGCCATGGACGTGGTCCTGCAGCCGAGCCCTGCCCTTAGCTGGAGGTCGACAGGTGGGATCCTGGACGTCTACATCTTCCTGGGCCCAGAGCCCAAGAGCGTGGTGCAGCAGTACCTGGACGTGGTGGGA |
| | TACCCGTTCATGCCGCCATACTGGGGCCTGGGCTTCCACCTGTGCCGCTGGGGCTACTCCTCCACCGCTATCACCCGCCAGGTGGTGGAGAACATGACCAGGGCCCACTTCCCCCTGGACGTCCAGTGGAACGATCTGGACTACA |
| | TGGACTCCCGGAGGGACTTCACGTTCAACAAGGATGGCTTCCGGGACTTCCCGGCCATGGTGCAGGAGCTGCACCAGGGCGGCCGGCGCTACATGATGATCGTGGATCCTGCCATCAGCAGCTCGGGCCCTGCCGGGAGCTACAG |
| | GCCCTACGACGAGGGTCTGCGGAGGGGGGTTTTCATCACCAACGAGACCGGCCAGCCGCTGATTGGGAAGGTGTGGCCCGGGTCCACTGCCTTCCCCGACTTCACCAACCCCACAGCCCTGGCCTGGTGGGAGGACATGGTGGCT |
| | GAGTTCCATGACCAGGTGCCCTTCGACGGCATGTGGATTGACATGAACGAGCCTTCCAACTTCATCAGAGGCTCTGAGGACGGCTGCCCCAACAACGAGCTGGAGAACCCACCCTACGTGCCTGGGGTGGTTGGGGGGACCCTCC |
| | AGGCGGCCACCATCTGTGCCTCCAGCCACCAGTTTCTCTCCACACACTACAACCTGCACAACCTCTACGGCCTGACCGAAGCCATCGCCTCCCACAGGGCGCTGGTGAAGGCTCGGGGGACACGCCCATTTGTGATCTCCCGCTC |
| | GACCTTTGCTGGCCACGGCCGATACGCCGGCCACTGGACGGGGGACGTGTGGAGCTCCTGGGAGCAGCTCGCCTCCTCCGTGCCAGAAATCCTGCAGTTTAACCTGCTGGGGGTGCCTCTGGTCGGGGCCGACGTCTGCGGCTTC |
| | CTGGGCAACACCTCAGAGGAGCTGTGTGTGCGCTGGACCCAGCTGGGGGCCTTCTACCCCTTCATGCGGAACCACAACAGCCTGCTCAGTCTGCCCCAGGAGCCGTACAGCTTCAGCGAGCCGGCCCAGCAGGCCATGAGGAAGG |
| | CCCTCACCCTGCGCTACGCACTCCTCCCCCACCTCTACACACTGTTCCACCAGGCCCACGTCGCGGGGGAGACCGTGGCCCGGCCCCTCTTCCTGGAGTTCCCCAAGGACTCTAGCACCTGGACTGTGGACCACCAGCTCCTGTG |
| | GGGGGAGGCCCTGCTCATCACCCCAGTGCTCCAGGCCGGGAAGGCCGAAGTGACTGGCTACTTCCCCTTGGGCACATGGTACGACCTGCAGACGGTGCCAATAGAGGCCCTTGGCAGCCTCCCACCCCCACCTGCAGCTCCCCGT |
| | GAGCCAGCCATCCACAGCGAGGGGCAGTGGGTGACGCTGCCGGCCCCCCTGGACACCATCAACGTGCACCTCCGGGCTGGGTACATCATCCCCCTGCAGGGCCCTGGCCTCACAACCACAGAGTCCCGCCAGCAGCCCATGGCCC |
| | TGGCTGTGGCCCTGACCAAGGGTGGAGAGGCCCGAGGGGAGCTGTTCTGGGACGACGGAGAGAGCCTGGAAGTGCTGGAGCGAGGGGCCTACACACAGGTCATCTTCCTGGCCAGGAATAACACGATCGTGAATGAGCTGGTACG |
| | TGTGACCAGTGAGGGAGCTGGCCTGCAGCTGCAGAAGGTGACTGTCCTGGGCGTGGCCACGGCGCCCCAGCAGGTCCTCTCCAACGGTGTCCCTGTCTCCAACTTCACCTACAGCCCCGACACCAAGGTCCTGGACATCTGTGTC |
| | TCGCTGTTGATGGGAGAGCAGTTTCTCGTCAGCTGGTGTTGATAACTCGAGCCGGGCGGAGTGTGTTAGTCTCTCCAGAGGGAGGCTGGTTCCCCAGGGAAGCAGAGCCTGTGTGCGGGCAGCAGCTGTGTGCGGGCCTGGGGGT |
| | TGTTAAGTGCAATTATTTTTAATAAAAGGGGCATTTGGAAAAAAAAAAAAAAGGTAGCAGTCGACAGATGAATTCTGCAGATCTGTGGCTTCTAGCTGCCCGGGTGGCATCCCTGTGACCCCTCCCCAGTGCCTCTCCTGGCCCT |
| | GGAAGTTGCCACTCCAGTGCCCACCAGCCTTGTCCTAATAAAATTAAGTTGCATCATTTTGTCTGACTAGGTGTCCTTCTATAATATTATGGGGTGGAGGGGGGTGGTATGGAGCAAGGGGCAAGTTGGGAAGACAACCTGAGTT |
| | GTTGGGATTCCAGGCATCGAGTAGATAAGTAGCATGGCGGGTTAATCATTAACTACAAGGAACCCCTAGTGATGGAGTTGGCCACTCCCTCTCTGCGCGCTCGCTCGCTCACTGAGGCCGGGCGACCAAAGGTCGCCCGACGCCC |
| | GGGCTTTGCCCGGGCGGCCTCAGTGAGCGAGCGAGCGCGCAGAGAGGGACAGATCCGAAGCCGGGCAAATCAGCGCCTGGCAGCAGTGGCGTCTGGCGGAAAACCTCAGTGTGACGCTCCCCGCCGCGTCCCACGCTTGTTCCCG |
| | GATCTGACCACCAGCGAAATCCGATTTTTGCACCGAGCTGGGTAATAAGCGTTGGCAATTTAACCGCCAGTCAGGCTTTCTTTCACAGTGTGGATTGGCGATAAAAAACAACTGCTGACGCCGCTGCGCGATCAGTTCACCCGTT |
| | CACCGCTGGATAACGACTTGGCGTAAGTGAAGCGACCCGTAAGACCCTAACGCCTGGGTCGAACGCTGGAAGGCGGCGGGCCAAACCAGGCCGAAGCAGCGTTGTTGCAGTTCACGGCAGATACACTTGCTGTTGCGGTGCTGAT |
| | TACGACCGCTCACTCGTGGCAGCAACAGGGGAAAACCTTATTTATCAGCCGGAAAACCTACCGGATTGTTGGTAGTGGTCAATAGGCGATTACCGTTGTGTTGAAGTGGCGAGCGATACACCGCTTCCGGCGCGGATTGGCCTGA |
| | ACTGCCAACTGGCGCAGGTAGCAGAGCGGGTAAACTGGCTCGGATTAGGGCCGCAAGAAAACTATCCCGACCGCCTTACTGCCGCCTGTTTTGACCGCTGGGATCTGCCAAGTCAGACAGTATAGCCCGTACGTCTTCCCGAGCG |
| | AAAACGGTCTGCGCTGCGGGACGCGCGAATTGAATTTGGCCCACACCAGTGGCGCGGCGACTTCCAGTTCAATATCAGCCGCTACAGTGAACAGCAACTGTTGGAAACCAGCCTTCGCCAACTGCTGCACGCGGAAGAAGGCACT |
| | GGCTGAATATCGACGGTTTCCAGTTGGGGATTGGTGGCGACGACTCCTGGAGCCCGTCAGTATCGGCGGACTTCCAACTGAGCGCCGGTCGCTACCTTACCAGTTGGTCTGGTGTCAAAAAGCGTCCGCTTGAGTCTAGCGATCG |
| | CGCGCAGATCTGTCATGTGAGCAAAAGGCCAGCAAAAGGCCAGGAACCGTAAAAAGGCCGCGTTGCTGGCGTTTTTCCATAGGCTCCGCCCCCCTGACGAGCATCACAAAAATCGACGCTCAAGTCAGAGGTGGCGAAACCCGAC |
| | AGGACTATAAAGATACCAGGCGTTTCCCCCTGGAAGCTCCCTCGTGCGCTCTCCTGTTCCGACCCTGCCGCTTACCGGATACCTGTCCGCCTTTCTCCCTTCGGGAAGCGTGGCGCTTTCTCATAGCTCACGCTGTAGGTATCTC |
| | AGTTCGGTGTAGGTCGTTCGCTCCAAGCTGGGCTGTGTGCACGAACCCCCCGTTCAGCCCGACCGCTGCGCCTTATCCGGTAACTATCGTCTTGAGTCCAACCCGGTAAGACACGACTTATCGCCACTGGCAGCAGCCACTGGTA |
| | ACAGGATTAGCAGAGCGAGGTATGTAGGCGGTGCTACAGAGTTCTTGAAGTGGTGGCCTAACTACGGCTACACTAGAAGAACAGTATTTGGTATCTGCGCTCTGCTGAAGCCAGTTACCTTCGGAAAAAGAGTTGGTAGCTCTTG |
| | ATCCGGCAAACAAACCACCGCTGGTAGCGGTGGTTTTTTTGTTTGCAAGCAGCAGATTACGCGCAGAAAAAAAGGATCTCAAGAAGATCCTTTGATCTTTTCTACGGGGTCTGACGCTCAGTGGAACGAAAACTCACGTTAAGGG |
| | ATTTTGGTCATGAGATTATCAAAAAGGATCTTCACCTAGATCCTTTTCACGTAGAAAGCCAGTCCGCAGAAACGGTGCTGACCCCGGATGAATGTCAGCTACTGGGCTATCTGGACAAGGGAAAACGCAAGCGCAAAGAGAAAGC |
| | AGGTAGCTTGCAGTGGGCTTACATGGCGATAGCTAGACTGGGCGGTTTTATGGACAGCAAGCGAACCGGAATTGCCAGCTGGGGCGCCCTCTGGTAAGGTTGGGAAGCCCTGCAAAGTAAACTGGATGGCTTTCTTGCCGCCAAG |
| | GATCTGATGGCGCAGGGGATCAAGATCTGATCAAGAGACAGGATGAGGATCGTTTCGCATGATTGAACAAGATGGATTGCACGCAGGTTCTCCGGCCGCTTGGGTGGAGAGGCTATTCGGCTATGACTGGGCACAACAGACAATC |
| | GGCTGCTCTGATGCCGCCGTGTTCCGGCTGTCAGCGCAGGGGCGCCCGGTTCTTTTTGTCAAGACCGACCTGTCCGGTGCCCTGAATGAACTGCAAGACGAGGCAGCGCGGCTATCGTGGCTGGCCACGACGGGCGTTCCTTGCG |
| | CAGCTGTGCTCGACGTTGTCACTGAAGCGGGAAGGGACTGGCTGCTATTGGGCGAAGTGCCGGGGCAGGATCTCCTGTCATCTCACCTTGCTCCTGCCGAGAAAGTATCCATCATGGCTGATGCAATGCGGCGGCTGCATACGCT |
| | TGATCCGGCTACCTGCCCATTCGACCACCAAGCGAAACATCGCATCGAGCGAGCACGTACTCGGATGGAAGCCGGTCTTGTCGATCAGGATGATCTGGACGAAGAGCATCAGGGGCTCGCGCCAGCCGAACTGTTCGCCAGGCTC |
| | AAGGCGAGCATGCCCGACGGCGAGGATCTCGTCGTGACCCATGGCGATGCCTGCTTGCCGAATATCATGGTGGAAAATGGCCGCTTTTCTGGATTCATCGACTGTGGCCGGCTGGGTGTGGCGGACCGCTATCAGGACATAGCGT |
| | TGGCTACCCGTGATATTGCTGAAGAGCTTGGCGGCGAATGGGCTGACCGCTTCCTCGTGCTTTACGGTATCGCCGCTCCCGATTCGCAGCGCATCGCCTTCTATCGCCTTCTTGACGAGTTCTTCTGAATTTAAAGCCCAATACG |
| | CAAACCGCCTCTCCCCGCGCGTTGGCCGAAGCGGTTCAATATTTTGTTAAAATTCGCGTTAAATTTTTGTTAAATCAGCTATTTTTTAACCAATAGGCCGAAATCGGCAAAATCCCTTGTAAATCAAAAGAATAGACCGAGATAG |
| | GGTTGAGTGTTGTTCCAGTTTGGAACAAGAGTCCACTATTAAAGAACGTGGACTCCAACGTCAAAGGGCGAAAAACCGTCTATCAGGGCGTTGGCCCACTACGTGAACCTTCACCCTAATCAAGTTTTTTGGGGTCGAGGTGCCG |
| | TAAAGCACTAAATCGGAACCCTAAAGGGAGCCCCCGATTTAGAGCTTGACGGGGAAACCGGCGAACGTGGCGAGAAAGGAAGGGAAGAAAGCGAAAGGAGCGGGCGCTAGGGCGCTGGCAAGTGTAGCGGTCACGCTGCGCGTAA |
| | CCACCACACCCGCCGCGCTAAGCGCCGCTACAGGGCGCGTCCCTTCGCCTTCAGGCTGCGTCGAGTACTGTACTGTGAGCCAGAGTTGCCCGGCGCTCTCCGGCTGCGGTAGTTCAGGCAGTTCAATCAACTGTTTACCTTGTGG |
| | AGCGACTCCAGAGGCACTTCACCGCTTGCCAGCGGCTTACGATCCAGCGCCACGATCCAGTGCAGGAGATCGTTATCGCTATACGGAACAGGTATTCGCTGGTCACTTCGATAAGGTTTGCCCGGATAAACGGAACTGGAAAAAC |
| | TGCTGCTGGTGTTTTGCTTCCGTCAGTGCTGGATCGGCGTGCGGTCGGCAAAGACCAGACCGTTCTAACAGAACTGGCGATTGTTCGGCGTATCGCCAAAATCACCGCCGTAAGCCGACCACGGGTTGCCGTTTTCAGCAGGATT |
| | TAATCAGCGACTGATCCACCCAGTCCCAGACGAAGCCGCCCTGTAAACGGGGATACTGACGAAACGCCTGCCAGTATTTAGCGAAACCGCCAAGACTGTTACCCAAGCGTGGGCGTATTCGCAAAGGATCAGCGGGCGCGTCTCT |
| | CCAGGTAGCGAAAGCCTTTTTTGATCGACCTTTCGGCACAGCCGGGAAGGGCTGGTCTTCAACCACGCGCGCGTACAACGGGCAAATAATATCGGTGGCCGTGGTGTCGGCTCCGCCGCCTTCAACTGCACCGGGCGGGAAGGAT |
| | CGACAGATTTGATCCAGCGATACAGCGCGTCGTGATTAGCGCCGTGGCCTGATTCAATTCCCCAGCGACCAGTAGATCACACTCGGGTGATTACGATTGCGCTGCACCAGTCGCGTTACGGTTCGCTCTTCGCCGGTAGCCAGCG |
| | CGGATCACGGTCAGACGATTCGTTGGCACGATCCGTGGGTTTCAATACTGGCTTCAAACCACCACTAACAGGCCGTAGCGGTCGCACAGCGTGTACCACAGCGGTTGGTTCGGATAATCGAACAGCGCACGGCGTTAAAGTTGTT |
| | CTGCTTCAACAGCAGGATATTCTGCACCTTCGTCTGCTCTTCCTAACCTGACCAAGCAGAGGATCTGCTCGTGACGGTTAATCCTCGAATCAGCAACGGCTTGCCGTTCAGCAGCAGCAGACCAAGTTCAATCCGCACCTCGCGG |
| | AAACCGACAACGCAGGCTTCTGCTTCAATCAGCGTGCCGTCGGCGGTGTGCAGTTCAACCACCGCACGATAGAGATTCGGGATTTCGGCGCTCCACAGTTTCGGGTTTTCGACGTTCAGACGTAGTGTGACGCGATCTGCAAACC |
| | ACCACGCTCAACGATAATTTCACCGCCGAAAGGCGCGGTGCCGCTGGCGACCTGCGTTTCACCCTGCCAGAAAGAAACTGTTACCCGTAGGTAGTCACGCAACTCGCCGCACACTGAACTTCAGCCTCCAGTACAGCGCGGCTGA |
| | AATCGTCTTAAAGCGAGTGGCAACTGGAAATCGCTGATTTGTGTAGTCGGTTTAGCAGCAACGAGACTTCACGGAAAATCCGCTAATCCGCCACAGATCCTGATCTTCCAGATAACTGCCGTCACTCCAACGCAGCACCTTCACC |
| | GCGAGGCGGTTTTCTCCGGCGCGTAAAAATCGCTCAGGTCAAATTCAGACGGCAAACGACTGTCCTGGCCGTAACCGACCCAGCGCCCGTTGCACCACAGATTGAAACGCCGAGTTTACGCCTCAAAAATAATTCGCGTCTGGCC |
| | TTCCTGTAGCCAGCTTTCACAACTATAATAGTGAGCGAGTAACAACCCGTCGGATTCTCCGTGGGAACAAACGGCGGATTGACCGTATAGGGATAGGTTACGTTGGTGTAGTAGGGCGCTCCGTAACCGTGCTACTGCCAGTTTG |
| | AGGGGACGACGACAGTATCGGCCTCAGGAAGATCGCACTCCAGCCAGCTTTCCGGCACCGCTTCTGGTACTGGAAACCAGGCAAAGCGCCTATCGCCTATCAGGCTGCACAACTGTTGGGAAGGGCGATCTGTGCGGGCCTCTTC |
| | GCTATTACGCCAGCTTGCGAAAGGGGGTAGTGCTGCAAGGCGATTAAGTTGGGTAACGCCAGGGTTTTCCCAGTCACGACGTGGATCTGGGCCACTCCCTCT |
| 463 | > AAV3B CAPSID NUCLEOTIDE SEQUENCE |
| | ATGGCTGCTGACGGTTATCTTCCAGATTGGCTCGAGGACAACCTTTCTGAAGGCATTCGTGAGTGGTGGGCTCTGAAACCTGGAGTCCCTCAACCCAAAGCGAACCAACAACACCAGGACAACCGTCGGGGTCTTGTGCTTCCGG |
| | GTTACAAATACCTCGGACCCGGTAACGGACTCGACAAAGGAGAGCCGGTCAACGAGGCGGACGCGGCAGCCCTCGAACACGACAAAGCTTACGACCAGCAGCTCAAGGCCGGTGACAACCCGTACCTCAAGTACAACCACGCCGA |
| | CGCCGAGTTTCAGGAGCGTCTTCAAGAAGATACGTCTTTTGGGGGCAACCTTGGCAGAGCAGTCTTCCAGGCCAAAAAGAGGATCCTTGAGCCTCTTGGTCTGGTTGAGGAAGCAGCTAAAACGGCTCCTGGAAAGAAGAGGCCT |
| | GTAGATCAGTCTCCTCAGGAACCGGACTCATCATCTGGTGTTGGCAAATCGGGCAAACAGCCTGCCAGAAAAAGACTAAATTTCGGTCAGACTGGCGACTCAGAGTCAGTCCCAGACCCTCAACCTCTCGGAGAACCACCAGCAG |
| | CCCCCACAAGTTTGGGATCTAATACAATGGCTTCAGGCGGTGGCGCACCAATGGCAGACAATAACGAGGGTGCCGATGGAGTGGGTAATTCCTCAGGAAATTGGCATTGCGATTCCCAATGGCTGGGCGACAGAGTCATCACCAC |
| | CAGCACCAGAACCTGGGCCCTGCCCACTTACAACAACCATCTCTACAAGCAAATCTCCAGCCAATCAGGAGCTTCAAACGACAACCACTACTTTGGCTACAGCACCCCTTGGGGGTATTTTGACTTTAACAGATTCCACTGCCAC |
| | TTCTCACCACGTGACTGGCAGCGACTCATTAACAACAACTGGGGATTCCGGCCCAAGAAACTCAGCTTCAAGCTCTTCAACATCCAAGTTAAAGAGGTCACGCAGAACGATGGCACGACGACTATTGCCAATAACCTTACCAGCA |
| | CGGTTCAAGTGTTTACGGACTCGGAGTATCAGCTCCCGTACGTGCTCGGGTCGGCGCACCAAGGCTGTCTCCCGCCGTTTCCAGCGGACGTCTTCATGGTCCCTCAGTATGGATACCTCACCCTGAACAACGGAAGTCAAGCGGT |
| | GGGACGCTCATCCTTTTACTGCCTGGAGTACTTCCCTTCGCAGATGCTAAGGACTGGAAATAACTTCCAATTCAGCTATACCTTCGAGGATGTACCTTTTCACAGCAGCTACGCTCACAGCCAGAGTTTGGATCGCTTGATGAAT |
| | CCTCTTATTGATCAGTATCTGTACTACCTGAACAGAACGCAAGGAACAACCTCTGGAACAACCAACCAATCACGGCTGCTTTTTAGCCAGGCTGGGCCTCAGTCTATGTCTTTGCAGGCCAGAAATTGGCTACCTGGGCCCTGCT |
| | ACCGGCAACAGAGACTTTCAAAGACTGCTAACGACAACAACAACAGTAACTTTCCTTGGACAGCGGCCAGCAAATATCATCTCAATGGCCGCGACTCGCTGGTGAATCCAGGACCAGCTATGGCCAGTCACAAGGACGATGAAGA |
| | AAAATTTTTCCCTATGCACGGCAATCTAATATTTGGCAAAGAAGGGACAACGGCAAGTAACGCAGAATTAGATAATGTAATGATTACGGATGAAGAAGAGATTCGTACCACCAATCCTGTGGCAACAGAGCAGTATGGAACTGTG |
| | GCAAATAACTTGCAGAGCTCAAATACAGCTCCCACGACTAGAACTGTCAATGATCAGGGGGCCTTACCTGGCATGGTGTGGCAAGATCGTGACGTGTACCTTCAAGGACCTATCTGGGCAAAGATTCCTCACACGGATGGACACT |
| | TTCATCCTTCTCCTCTGATGGGAGGCTTTGGACTGAAACATCCGCCTCCTCAAATCATGATCAAAAATACTCCGGTACCGGCAAATCCTCCGACGACTTTCAGCCCGGCCAAGTTTGCTTCATTTATCACTCAGTACTCCACTGG |
| | ACAGGTCAGCGTGGAAATTGAGTGGGAGCTACAGAAAGAAAACAGCAAACGTTGGAATCCAGAGATTCAGTACACTTCCAACTACAACAAGTCTGTTAATGTGGACTTTACTGTAGACACTAATGGTGTTTATAGTGAACCTCGC |
| | CCTATTGGAACCCGGTATCTCACACGAAACTTG |
| 452 | > AAV3B CAPSID AMINO ACID SEQUENCE |
| | MAADGYLPDWLEDNLSEGIREWWALKPGVPQPKANQQHQDNRRGLVLPGYKYLGPGNGLDKGEPVNEADAAALEHDKAYDQQLKAGDNPYLKYNHADAEFQERLQEDTSFGGNLGRAVFQAKKRILEPLGLVEEAAKTAPGKKRP |
| | VDQSPQEPDSSSGVGKSGKQPARKRLNFGQTGDSESVPDPQPLGEPPAAPTSLGSNTMASGGGAPMADNNEGADGVGNSSGNWHCDSQWLGDRVITTSTRTWALPTYNNHLYKQISSQSGASNDNHYFGYSTPWGYFDFNRFHCH |
| | FSPRDWQRLINNNWGFRPKKLSFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFQFSYTFEDVPFHSSYAHSQSLDRLMN |
| | PLIDQYLYYLNRTQGTTSGTTNQSRLLFSQAGPQSMSLQARNWLPGPCYRQQRLSKTANDNNNSNFPWTAASKYHLNGRDSLVNPGPAMASHKDDEEKFFPMHGNLIFGKEGTTASNAELDNVMITDEEEIRTTNPVATEQYGTV |
| | ANNLQSSNTAPTTRTVNDQGALPGMVWQDRDVYLQGPIWAKIPHTDGHFHPSPLMGGFGLKHPPPQIMIKNTPVPANPPTTFSPAKFASFITQYSTGQVSVEIEWELQKENSKRWNPEIQYTSNYNKSVNVDFTVDTNGVYSEPR |
| | PIGTRYLTRNL |
| 453 | > AAV3B 265D CAPSID NUCLEOTIDE SEQUENCE |
| | ATGGCTGCTGACGGTTATCTTCCAGATTGGCTCGAGGACAACCTTTCTGAAGGCATTCGTGAGTGGTGGGCTCTGAAACCTGGAGTCCCTCAACCCAAAGCGAACCAACAACACCAGGACAACCGTCGGGGTCTTGTGCTTCCGG |
| | GTTACAAATACCTCGGACCCGGTAACGGACTCGACAAAGGAGAGCCGGTCAACGAGGCGGACGCGGCAGCCCTCGAACACGACAAAGCTTACGACCAGCAGCTCAAGGCCGGTGACAACCCGTACCTCAAGTACAACCACGCCGA |
| | CGCCGAGTTTCAGGAGCGTCTTCAAGAAGATACGTCTTTTGGGGGCAACCTTGGCAGAGCAGTCTTCCAGGCCAAAAAGAGGATCCTTGAGCCTCTTGGTCTGGTTGAGGAAGCAGCTAAAACGGCTCCTGGAAAGAAGAGGCCT |
| | GTAGATCAGTCTCCTCAGGAACCGGACTCATCATCTGGTGTTGGCAAATCGGGCAAACAGCCTGCCAGAAAAAGACTAAATTTCGGTCAGACTGGCGACTCAGAGTCAGTCCCAGACCCTCAACCTCTCGGAGAACCACCAGCAG |
| | CCCCCACAAGTTTGGGATCTAATACAATGGCTTCAGGCGGTGGCGCACCAATGGCAGACAATAACGAGGGTGCCGATGGAGTGGGTAATTCCTCAGGAAATTGGCATTGCGATTCCCAATGGCTGGGCGACAGAGTCATCACCAC |
| | CAGCACCAGAACCTGGGCCCTGCCCACTTACAACAACCATCTCTACAAGCAAATCTCCAGCCAATCAGATGCTTCAAACGACAACCACTACTTTGGCTACAGCACCCCTTGGGGGTATTTTGACTTTAACAGATTCCACTGCCAC |
| | TTCTCACCACGTGACTGGCAGCGACTCATTAACAACAACTGGGGATTCCGGCCCAAGAAACTCAGCTTCAAGCTCTTCAACATCCAAGTTAAAGAGGTCACGCAGAACGATGGCACGACGACTATTGCCAATAACCTTACCAGCA |
| | CGGTTCAAGTGTTTACGGACTCGGAGTATCAGCTCCCGTACGTGCTCGGGTCGGCGCACCAAGGCTGTCTCCCGCCGTTTCCAGCGGACGTCTTCATGGTCCCTCAGTATGGATACCTCACCCTGAACAACGGAAGTCAAGCGGT |
| | GGGACGCTCATCCTTTTACTGCCTGGAGTACTTCCCTTCGCAGATGCTAAGGACTGGAAATAACTTCCAATTCAGCTATACCTTCGAGGATGTACCTTTTCACAGCAGCTACGCTCACAGCCAGAGTTTGGATCGCTTGATGAAT |
| | CCTCTTATTGATCAGTATCTGTACTACCTGAACAGAACGCAAGGAACAACCTCTGGAACAACCAACCAATCACGGCTGCTTTTTAGCCAGGCTGGGCCTCAGTCTATGTCTTTGCAGGCCAGAAATTGGCTACCTGGGCCCTGCT |
| | ACCGGCAACAGAGACTTTCAAAGACTGCTAACGACAACAACAACAGTAACTTTCCTTGGACAGCGGCCAGCAAATATCATCTCAATGGCCGCGACTCGCTGGTGAATCCAGGACCAGCTATGGCCAGTCACAAGGACGATGAAGA |
| | AAAATTTTTCCCTATGCACGGCAATCTAATATTTGGCAAAGAAGGGACAACGGCAAGTAACGCAGAATTAGATAATGTAATGATTACGGATGAAGAAGAGATTCGTACCACCAATCCTGTGGCAACAGAGCAGTATGGAACTGTG |
| | GCAAATAACTTGCAGAGCTCAAATACAGCTCCCACGACTAGAACTGTCAATGATCAGGGGGCCTTACCTGGCATGGTGTGGCAAGATCGTGACGTGTACCTTCAAGGACCTATCTGGGCAAAGATTCCTCACACGGATGGACACT |
| | TTCATCCTTCTCCTCTGATGGGAGGCTTTGGACTGAAACATCCGCCTCCTCAAATCATGATCAAAAATACTCCGGTACCGGCAAATCCTCCGACGACTTTCAGCCCGGCCAAGTTTGCTTCATTTATCACTCAGTACTCCACTGG |
| | ACAGGTCAGCGTGGAAATTGAGTGGGAGCTACAGAAAGAAAACAGCAAACGTTGGAATCCAGAGATTCAGTACACTTCCAACTACAACAAGTCTGTTAATGTGGACTTTACTGTAGACACTAATGGTGTTTATAGTGAACCTCGC |
| | CCTATTGGAACCCGGTATCTCACACGAAACTTG |
| 454 | > AAV3B 265D CAPSID AMINO ACID |
| | MAADGYLPDWLEDNLSEGIREWWALKPGVPQPKANQQHQDNRRGLVLPGYKYLGPGNGLDKGEPVNEADAAALEHDKAYDQQLKAGDNPYLKYNHADAEFQERLQEDTSFGGNLGRAVFQAKKRILEPLGLVEEAAKTAPGKKRP |
| | VDQSPQEPDSSSGVGKSGKQPARKRLNFGQTGDSESVPDPQPLGEPPAAPTSLGSNTMASGGGAPMADNNEGADGVGNSSGNWHCDSQWLGDRVITTSTRIWALPTYNNHLYKQISSQSDASNDNHYFGYSTPWGYFFENRFHCH |
| | FSPRDWQRLINNNWGFRPKKLSFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFQFSYTFEDVPFHSSYAHSQSLDRLMN |
| | PLIDQYLYYLNRTQGTTSGTTNQSRLLFSQAGPQSMSLQARNWLPGPCYRQQRLSKTANDNNNSNFPWTAASKYHLNGRDSLVNPGPAMASHKDDEEKFFPMHGNLIFGKEGTTASNAELDNVMITDEEEIRTTNPVATEQYGTV |
| | ANNLQSSNTAPTTRTVNDQGALPGMVWQDRDVYLQGPIWAKIPHTDGHFHPSPLMGGFGLKHPPPQIMIKNTPVPANPPTTFSPAKFASFITQYSTGQVSVEIEWELQKENSKRWNPEIQYTSNYNKSVNVDFTVDTNGVYSEPR |
| | PIGTRYLTRNL |
| 455 | > AAV3B ST (S663V + T492V) CAPSID NUCLEOTIDE SEQUENCE |
| | ATGGCTGCTGACGGTTATCTTCCAGATTGGCTCGAGGACAACCTTTCTGAAGGCATTCGTGAGTGGTGGGCTCTGAAACCTGGAGTCCCTCAACCCAAAGCGAACCAACAACACCAGGACAACCGTCGGGGTCTTGTGCTTCCGG |
| | GTTACAAATACCTCGGACCCGGTAACGGACTCGACAAAGGAGAGCCGGTCAACGAGGCGGACGCGGCAGCCCTCGAACACGACAAAGCTTACGACCAGCAGCTCAAGGCCGGTGACAACCCGTACCTCAAGTACAACCACGCCGA |
| | CGCCGAGTTTCAGGAGCGTCTTCAAGAAGATACGTCTTTTGGGGGCAACCTTGGCAGAGCAGTCTTCCAGGCCAAAAAGAGGATCCTTGAGCCTCTTGGTCTGGTTGAGGAAGCAGCTAAAACGGCTCCTGGAAAGAAGAGGCCT |
| | GTAGATCAGTCTCCTCAGGAACCGGACTCATCATCTGGTGTTGGCAAATCGGGCAAACAGCCTGCCAGAAAAAGACTAAATTTCGGTCAGACTGGCGACTCAGAGTCAGTCCCAGACCCTCAACCTCTCGGAGAACCACCAGCAG |
| | CCCCCACAAGTTTGGGATCTAATACAATGGCTTCAGGCGGTGGCGCACCAATGGCAGACAATAACGAGGGTGCCGATGGAGTGGGTAATTCCTCAGGAAATTGGCATTGCGATTCCCAATGGCTGGGCGACAGAGTCATCACCAC |
| | CAGCACCAGAACCTGGGCCCTGCCCACTTACAACAACCATCTCTACAAGCAAATCTCCAGCCAATCAGATGCTTCAAACGACAACCACTACTTTGGCTACAGCACCCCTTGGGGGTATTTTGACTTTAACAGATTCCACTGCCAC |
| | TTCTCACCACGTGACTGGCAGCGACTCATTAACAACAACTGGGGATTCCGGCCCAAGAAACTCAGCTTCAAGCTCTTCAACATCCAAGTTAAAGAGGTCACGCAGAACGATGGCACGACGACTATTGCCAATAACCTTACCAGCA |
| | CGGTTCAAGTGTTTACGGACTCGGAGTATCAGCTCCCGTACGTGCTCGGGTCGGCGCACCAAGGCTGTCTCCCGCCGTTTCCAGCGGACGTCTTCATGGTCCCTCAGTATGGATACCTCACCCTGAACAACGGAAGTCAAGCGGT |
| | GGGACGCTCATCCTTTTACTGCCTGGAGTACTTCCCTTCGCAGATGCTAAGGACTGGAAATAACTTCCAATTCAGCTATACCTTCGAGGATGTACCTTTTCACAGCAGCTACGCTCACAGCCAGAGTTTGGATCGCTTGATGAAT |
| | CCTCTTATTGATCAGTATCTGTACTACCTGAACAGAACGCAAGGAACAACCTCTGGAACAACCAACCAATCACGGCTGCTTTTTAGCCAGGCTGGGCCTCAGTCTATGTCTTTGCAGGCCAGAAATTGGCTACCTGGGCCCTGCT |
| | ACCGGCAACAGAGACTTTCAAAGGTAGCTAACGACAACAACAACAGTAACTTTCCTTGGACAGCGGCCAGCAAATATCATCTCAATGGCCGCGACTCGCTGGTGAATCCAGGACCAGCTATGGCCAGTCACAAGGACGATGAAGA |
| | AAAATTTTTCCCTATGCACGGCAATCTAATATTTGGCAAAGAAGGGACAACGGCAAGTAACGCAGAATTAGATAATGTAATGATTACGGATGAAGAAGAGATTCGTACCACCAATCCTGTGGCAACAGAGCAGTATGGAACTGTG |
| | GCAAATAACTTGCAGAGCTCAAATACAGCTCCCACGACTAGAACTGTCAATGATCAGGGGGCCTTACCTGGCATGGTGTGGCAAGATCGTGACGTGTACCTTCAAGGACCTATCTGGGCAAAGATTCCTCACACGGATGGACACT |
| | TTCATCCTTCTCCTCTGATGGGAGGCTTTGGACTGAAACATCCGCCTCCTCAAATCATGATCAAAAATACTCCGGTACCGGCAAATCCTCCGACGACTTTCGTACCGGCCAAGTTTGCTTCATTTATCACTCAGTACTCCACTGG |
| | ACAGGTCAGCGTGGAAATTGAGTGGGAGCTACAGAAAGAAAACAGCAAACGTTGGAATCCAGAGATTCAGTACACTTCCAACTACAACAAGTCTGTTAATGTGGACTTTACTGTAGACACTAATGGTGTTTATAGTGAACCTCGC |
| | CCTATTGGAACCCGGTATCTCACACGAAACTTG |
| 456 | > AAV3B ST (S663V + T492V) AMINO ACID SEQUENCE |
| | MAADGYLPDWLEDNLSEGIREWWALKPGVPQPKANQQHQDNRRGLVLPGYKYLGPGNGLDKGEPVNEADAAALEHDKAYDQQLKAGDNPYLKYNHADAEFQERLQEDTSFGGNLGRAVFQAKKRILEPLGLVEEAAKTAPGKKRP |
| | VDQSPQEPDSSSGVGKSGKQPARKRLNFGQTGDSESVPDPQPLGEPPAAPTSLGSNTMASGGGAPMADNNEGADGVGNSSGNWHCDSQWLGDRVITTSTRTWALPTYNNHLYKQISSQSGASNDNHYFGYSTPWGYFDFNRFHCH |
| | FSPRDWQRLINNNWGFRPKKLSFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFQFSYTFEDVPFHSSYAHSQSLDRLMN |
| | PLIDQYLYYLNRTQGTTSGTTNQSRLLFSQAGPQSMSLQARNWLPGPCYRQQRLSKVANDNNNSNFPWTAASKYHLNGRDSLVNPGPAMASHKDDEEKFFPMHGNLIFGKEGTTASNAELDNVMITDEEEIRTTNPVATEQYGTV |
| | ANNLQSSNTAPTTRTVNDQGALPGMVWQDRDVYLQGPIWAKIPHTDGHFHPSPLMGGFGLKHPPPQIMIKNTPVPANPPTTFVPAKFASFITQYSTGQVSVEIEWELQKENSKRWNPEIQYTSNYNKSVNVDFTVDTNGVYSEPR |
| | PIGTRYLTRNL |
| 457 | > AAV3B265D549A CAPSID NUCLEOTIDE SEQUENCE |
| | GAAACCTGGAGTCCCTCAACCCAAAGCGAACCAACAACACCAGGACAACCGTCGGGGTCTTGTGCTTCCGGGTTACAAATACCTCGGACCCGGTAACGGACTCGACAAAGGAGAGCCGGTCAACGAGGCGGACGCGGCAGCCCTC |
| | GAACACGACAAAGCTTACGACCAGCAGCTCAAGGCCGGTGACAACCCGTACCTCAAGTACAACCACGCCGACGCCGAGTTTCAGGAGCGTCTTCAAGAAGATACGTCTTTTGGGGGCAACCTTGGCAGAGCAGTCTTCCAGGCCA |
| | AAAAGAGGATCCTTGAGCCTCTTGGTCTGGTTGAGGAAGCAGCTAAAACGGCTCCTGGAAAGAAGAGGCCTGTAGATCAGTCTCCTCAGGAACCGGACTCATCATCTGGTGTTGGCAAATCGGGCAAACAGCCTGCCAGAAAAAG |
| | ACTAAATTTCGGTCAGACTGGCGACTCAGAGTCAGTCCCAGACCCTCAACCTCTCGGAGAACCACCAGCAGCCCCCACAAGTTTGGGATCTAATACAATGGCTTCAGGCGGTGGCGCACCAATGGCAGACAATAACGAGGGTGCC |
| | GATGGAGTGGGTAATTCCTCAGGAAATTGGCATTGCGATTCCCAATGGCTGGGCGACAGAGTCATCACCACCAGCACCAGAACCTGGGCCCTGCCCACTTACAACAACCATCTCTACAAGCAAATCTCCAGCCAATCAGGAGCTT |
| | CAAACGACAACCACTACTTTGGCTACAGCACCCCTTGGGGGTATTTTGACTTTAACAGATTCCACTGCCACTTCTCACCACGTGACTGGCAGCGACTCATTAACAACAACTGGGGATTCCGGCCCAAGAAACTCAGCTTCAAGCT |
| | CTTCAACATCCAAGTTAAAGAGGTCACGCAGAACGATGGCACGACGACTATTGCCAATAACCTTACCAGCACGGTTCAAGTGTTTACGGACTCGGAGTATCAGCTCCCGTACGTGCTCGGGTCGGCGCACCAAGGCTGTCTCCCG |
| | CCGTTTCCAGCGGACGTCTTCATGGTCCCTCAGTATGGATACCTCACCCTGAACAACGGAAGTCAAGCGGTGGGACGCTCATCCTTTTACTGCCTGGAGTACTTCCCTTCGCAGATGCTAAGGACTGGAAATAACTTCCAATTCA |
| | GCTATACCTTCGAGGATGTACCTTTTCACAGCAGCTACGCTCACAGCCAGAGTTTGGATCGCTTGATGAATCCTCTTATTGATCAGTATCTGTACTACCTGAACAGAACGCAAGGAACAACCTCTGGAACAACCAACCAATCACG |
| | GCTGCTTTTTAGCCAGGCTGGGCCTCAGTCTATGTCTTTGCAGGCCAGAAATTGGCTACCTGGGCCCTGCTACCGGCAACAGAGACTTTCAAAGACTGCTAACGACAACAACAACAGTAACTTTCCTTGGACAGCGGCCAGCAAA |
| | TATCATCTCAATGGCCGCGACTCGCTGGTGAATCCAGGACCAGCTATGGCCAGTCACAAGGACGATGAAGAAAAATTTTTCCCTATGCACGGCAATCTAATATTTGGCAAAGAAGGGACAACGGCAAGTAACGCAGAATTAGATA |
| | ATGTAATGATTACGGATGAAGAAGAGATTCGTACCACCAATCCTGTGGCAACAGAGCAGTATGGAACTGTGGCAAATAACTTGCAGAGCTCAAATACAGCTCCCACGACTAGAACTGTCAATGATCAGGGGGCCTTACCTGGCAT |
| | GGTGTGGCAAGATCGTGACGTGTACCTTCAAGGACCTATCTGGGCAAAGATTCCTCACACGGATGGACACTTTCATCCTTCTCCTCTGATGGGAGGCTTTGGACTGAAACATCCGCCTCCTCAAATCATGATCAAAAATACTCCG |
| | GTACCGGCAAATCCTCCGACGACTTTCAGCCCGGCCAAGTTTGCTTCATTTATCACTCAGTACTCCACTGGACAGGTCAGCGTGGAAATTGAGTGGGAGCTACAGAAAGAAAACAGCAAACGTTGGAATCCAGAGATTCAGTACA |
| | CTTCCAACTACAACAAGTCTGTTAATGTGGACTTTACTGTAGACACTAATGGTGTTTATAGTGAACCTCGCCCTATTGGAACCCGGTATCTCACACGAAACTTG |
| 458 | > AAV3B265D549A CAPSID AMINO ACID SEQUENCE |
| | MAADGYLPDWLEDNLSEGIREWWALKPGVPQPKANQQHQDNRRGLVLPGYKYLGPGNGLDKGEPVNEADAAALEHDKAYDQQLKAGDNPYLKYNHADAEFQERLQEDTSFGGNLGRAVFQAKKRILEPLGLVEEAAKTAPGKKRP |
| | VDQSPQEPDSSSGVGKSGKQPARKRLNFGQTGDSESVPDPQPLGEPPAAPTSLGSNTMASGGGAPMADNNEGADGVGNSSGNWHCDSQWLGDRVITTSTRTWALPTYNNHLYKQISSQSDASNDNHYFGYSTPWGYFDFNRFHCH |
| | FSPRDWQRLINNNWGFRPKKLSFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFQFSYTFEDVPFHSSYAHSQSLDRLMN |
| | PLIDQYLYYLNRTQGTTSGTTNQSRLLFSQAGPQSMSLQARNWLPGPCYRQQRLSKTANDNNNSNFPWTAASKYHLNGRDSLVNPGPAMASHKDDEEKFFPMHGNLIFGKEGTAASNAELDNVMITDEEEIRTTNPVATEQYGTV |
| | ANNLQSSNTAPTTRTVNDQGALPGMVWQDRDVYLQGPIWAKIPHTDGHFHPSPLMGGFGLKHPPPQIMIKNTPVPANPPTTFSPAKFASFITQYSTGQVSVEIEWELQKENSKRWNPEIQYTSNYNKSVNVDFTVDTNGVYSEPR |
| | PIGTRYLTRNL |
| 459 | > AAV3B549A CAPSID NUCLEOTIDE SEQUENCE |
| | ATGGCTGCTGACGGTTATCTTCCAGATTGGCTCGAGGACAACCTTTCTGAAGGCATTCGTGAGTGGTGGGCTCTGAAACCTGGAGTCCCTCAACCCAAAGCGAACCAACAACACCAGGACAACCGTCGGGGTCTTGTGCTTCCGG |
| | GTTACAAATACCTCGGACCCGGTAACGGACTCGACAAAGGAGAGCCGGTCAACGAGGCGGACGCGGCAGCCCTCGAACACGACAAAGCTTACGACCAGCAGCTCAAGGCCGGTGACAACCCGTACCTCAAGTACAACCACGCCGA |
| | CGCCGAGTTTCAGGAGCGTCTTCAAGAAGATACGTCTTTTGGGGGCAACCTTGGCAGAGCAGTCTTCCAGGCCAAAAAGAGGATCCTTGAGCCTCTTGGTCTGGTTGAGGAAGCAGCTAAAACGGCTCCTGGAAAGAAGAGGCCT |
| | GTAGATCAGTCTCCTCAGGAACCGGACTCATCATCTGGTGTTGGCAAATCGGGCAAACAGCCTGCCAGAAAAAGACTAAATTTCGGTCAGACTGGCGACTCAGAGTCAGTCCCAGACCCTCAACCTCTCGGAGAACCACCAGCAG |
| | CCCCCACAAGTTTGGGATCTAATACAATGGCTTCAGGCGGTGGCGCACCAATGGCAGACAATAACGAGGGTGCCGATGGAGTGGGTAATTCCTCAGGAAATTGGCATTGCGATTCCCAATGGCTGGGCGACAGAGTCATCACCAC |
| | CAGCACCAGAACCTGGGCCCTGCCCACTTACAACAACCATCTCTACAAGCAAATCTCCAGCCAATCAGGAGCTTCAAACGACAACCACTACTTTGGCTACAGCACCCCTTGGGGGTATTTTGACTTTAACAGATTCCACTGCCAC |
| | TTCTCACCACGTGACTGGCAGCGACTCATTAACAACAACTGGGGATTCCGGCCCAAGAAACTCAGCTTCAAGCTCTTCAACATCCAAGTTAAAGAGGTCACGCAGAACGATGGCACGACGACTATTGCCAATAACCTTACCAGCA |
| | CGGTTCAAGTGTTTACGGACTCGGAGTATCAGCTCCCGTACGTGCTCGGGTCGGCGCACCAAGGCTGTCTCCCGCCGTTTCCAGCGGACGTCTTCATGGTCCCTCAGTATGGATACCTCACCCTGAACAACGGAAGTCAAGCGGT |
| | GGGACGCTCATCCTTTTACTGCCTGGAGTACTTCCCTTCGCAGATGCTAAGGACTGGAAATAACTTCCAATTCAGCTATACCTTCGAGGATGTACCTTTTCACAGCAGCTACGCTCACAGCCAGAGTTTGGATCGCTTGATGAAT |
| | CCTCTTATTGATCAGTATCTGTACTACCTGAACAGAACGCAAGGAACAACCTCTGGAACAACCAACCAATCACGGCTGCTTTTTAGCCAGGCTGGGCCTCAGTCTATGTCTTTGCAGGCCAGAAATTGGCTACCTGGGCCCTGCT |
| | ACCGGCAACAGAGACTTTCAAAGACTGCTAACGACAACAACAACAGTAACTTTCCTTGGACAGCGGCCAGCAAATATCATCTCAATGGCCGCGACTCGCTGGTGAATCCAGGACCAGCTATGGCCAGTCACAAGGACGATGAAGA |
| | AAAATTTTTCCCTATGCACGGCAATCTAATATTTGGCAAAGAAGGGACAACGGCAAGTAACGCAGAATTAGATAATGTAATGATTACGGATGAAGAAGAGATTCGTACCACCAATCCTGTGGCAACAGAGCAGTATGGAACTGTG |
| | GCAAATAACTTGCAGAGCTCAAATACAGCTCCCACGACTAGAACTGTCAATGATCAGGGGGCCTTACCTGGCATGGTGTGGCAAGATCGTGACGTGTACCTTCAAGGACCTATCTGGGCAAAGATTCCTCACACGGATGGACACT |
| | TTCATCCTTCTCCTCTGATGGGAGGCTTTGGACTGAAACATCCGCCTCCTCAAATCATGATCAAAAATACTCCGGTACCGGCAAATCCTCCGACGACTTTCAGCCCGGCCAAGTTTGCTTCATTTATCACTCAGTACTCCACTGG |
| | ACAGGTCAGCGTGGAAATTGAGTGGGAGCTACAGAAAGAAAACAGCAAACGTTGGAATCCAGAGATTCAGTACACTTCCAACTACAACAAGTCTGTTAATGTGGACTTTACTGTAGACACTAATGGTGTTTATAGTGAACCTCGC |
| | CCTATTGGAACCCGGTATCTCACACGAAACTTG |
| 460 | > AAV3B549A CAPSID AMINO ACID SEQUENCE |
| | MAADGYLPDWLEDNLSEGIREWWALKPGVPQPKANQQHQDNRRGLVLPGYKYLGPGNGLDKGEPVNEADAAALEHDKAYDQQLKAGDNPYLKYNHADAEFQERLQEDTSFGGNLGRAVFQAKKRILEPLGLVEEAAKTAPGKKRP |
| | VDQSPQEPDSSSGVGKSGKQPARKRLNFGQTGDSESVPDPQPLGEPPAAPTSLGSNTMASGGGAPMADNNEGADGVGNSSGNWHCDSQWLGDRVITTSTRTWALPTYNNHLYKQISSQSGASNDNHYFGYSTPWGYFDFNRFHCH |
| | FSPRDWQRLINNNWGFRPKKLSFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFQFSYTFEDVPFHSSYAHSQSLDRLMN |
| | PLIDQYLYYLNRTQGTTSGTTNQSRLLFSQAGPQSMSLQARNWLPGPCYRQQRLSKTANDNNNSNFPWTAASKYHLNGRDSLVNPGPAMASHKDDEEKFFPMHGNLIFGKEGTAASNAELDNVMITDEEEIRTTNPVATEQYGTV |
| | ANNLQSSNTAPTTRTVNDQGALPGMVWQDRDVYLQGPIWAKIPHTDGHFHPSPLMGGFGLKHPPPQIMIKNTPVPANPPTTFSPAKFASFITQYSTGQVSVEIEWELQKENSKRWNPEIQYTSNYNKSVNVDFTVDTNGVYSEPR |
| | PIGTRYLTRNL |
| 461 | > AAV3BQ263Y CAPSID NUCLEOTIDE SEQUENCE |
| | ATGGCTGCTGACGGTTATCTTCCAGATTGGCTCGAGGACAACCTTTCTGAAGGCATTCGTGAGTGGTGGGCTCTGAAACCTGGAGTCCCTCAACCCAAAGCGAACCAACAACACCAGGACAACCGTCGGGGTCTTGTGCTTCCGG |
| | GTTACAAATACCTCGGACCCGGTAACGGACTCGACAAAGGAGAGCCGGTCAACGAGGCGGACGCGGCAGCCCTCGAACACGACAAAGCTTACGACCAGCAGCTCAAGGCCGGTGACAACCCGTACCTCAAGTACAACCACGCCGA |
| | CGCCGAGTTTCAGGAGCGTCTTCAAGAAGATACGTCTTTTGGGGGCAACCTTGGCAGAGCAGTCTTCCAGGCCAAAAAGAGGATCCTTGAGCCTCTTGGTCTGGTTGAGGAAGCAGCTAAAACGGCTCCTGGAAAGAAGAGGCCT |
| | GTAGATCAGTCTCCTCAGGAACCGGACTCATCATCTGGTGTTGGCAAATCGGGCAAACAGCCTGCCAGAAAAAGACTAAATTTCGGTCAGACTGGCGACTCAGAGTCAGTCCCAGACCCTCAACCTCTCGGAGAACCACCAGCAG |
| | CCCCCACAAGTTTGGGATCTAATACAATGGCTTCAGGCGGTGGCGCACCAATGGCAGACAATAACGAGGGTGCCGATGGAGTGGGTAATTCCTCAGGAAATTGGCATTGCGATTCCCAATGGCTGGGCGACAGAGTCATCACCAC |
| | CAGCACCAGAACCTGGGCCCTGCCCACTTACAACAACCATCTCTACAAGCAAATCTCCAGCCAATCAGGAGCTTCAAACGACAACCACTACTTTGGCTACAGCACCCCTTGGGGGTATTTTGACTTTAACAGATTCCACTGCCAC |
| | TTCTCACCACGTGACTGGCAGCGACTCATTAACAACAACTGGGGATTCCGGCCCAAGAAACTCAGCTTCAAGCTCTTCAACATCCAAGTTAAAGAGGTCACGCAGAACGATGGCACGACGACTATTGCCAATAACCTTACCAGCA |
| | CGGTTCAAGTGTTTACGGACTCGGAGTATCAGCTCCCGTACGTGCTCGGGTCGGCGCACCAAGGCTGTCTCCCGCCGTTTCCAGCGGACGTCTTCATGGTCCCTCAGTATGGATACCTCACCCTGAACAACGGAAGTCAAGCGGT |
| | GGGACGCTCATCCTTTTACTGCCTGGAGTACTTCCCTTCGCAGATGCTAAGGACTGGAAATAACTTCCAATTCAGCTATACCTTCGAGGATGTACCTTTTCACAGCAGCTACGCTCACAGCCAGAGTTTGGATCGCTTGATGAAT |
| | CCTCTTATTGATCAGTATCTGTACTACCTGAACAGAACGCAAGGAACAACCTCTGGAACAACCAACCAATCACGGCTGCTTTTTAGCCAGGCTGGGCCTCAGTCTATGTCTTTGCAGGCCAGAAATTGGCTACCTGGGCCCTGCT |
| | ACCGGCAACAGAGACTTTCAAAGACTGCTAACGACAACAACAACAGTAACTTTCCTTGGACAGCGGCCAGCAAATATCATCTCAATGGCCGCGACTCGCTGGTGAATCCAGGACCAGCTATGGCCAGTCACAAGGACGATGAAGA |
| | AAAATTTTTCCCTATGCACGGCAATCTAATATTTGGCAAAGAAGGGACAACGGCAAGTAACGCAGAATTAGATAATGTAATGATTACGGATGAAGAAGAGATTCGTACCACCAATCCTGTGGCAACAGAGCAGTATGGAACTGTG |
| | GCAAATAACTTGCAGAGCTCAAATACAGCTCCCACGACTAGAACTGTCAATGATCAGGGGGCCTTACCTGGCATGGTGTGGCAAGATCGTGACGTGTACCTTCAAGGACCTATCTGGGCAAAGATTCCTCACACGGATGGACACT |
| | TTCATCCTTCTCCTCTGATGGGAGGCTTTGGACTGAAACATCCGCCTCCTCAAATCATGATCAAAAATACTCCGGTACCGGCAAATCCTCCGACGACTTTCAGCCCGGCCAAGTTTGCTTCATTTATCACTCAGTACTCCACTGG |
| | ACAGGTCAGCGTGGAAATTGAGTGGGAGCTACAGAAAGAAAACAGCAAACGTTGGAATCCAGAGATTCAGTACACTTCCAACTACAACAAGTCTGTTAATGTGGACTTTACTGTAGACACTAATGGTGTTTATAGTGAACCTCGC |
| | CCTATTGGAACCCGGTATCTCACACGAAACTTG |
| 462 | > AAV3BQ263Y CAPSID AMINO ACID SEQUENCE |
| | MAADGYLPDWLEDNLSEGIREWWALKPGVPQPKANQQHQDNRRGLVLPGYKYLGPGNGLDKGEPVNEADAAALEHDKAYDQQLKAGDNPYLKYNHADAEFQERLQEDTSFGGNLGRAVFQAKKRILEPLGLVEEAAKTAPGKKRP |
| | VDQSPQEPDSSSGVGKSGKQPARKRLNFGQTGDSESVPDPQPLGEPPAAPTSLGSNTMASGGGAPMADNNEGADGVGNSSGNWHCDSQWLGDRVITTSTRTWALPTYNNHLYKQISSYSGASNDNHYFGYSTPWGYFDFNRFHCH |
| | FSPRDWQRLINNNWGFRPKKLSFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFQFSYTFEDVPFHSSYAHSQSLDRLMN |
| | PLIDQYLYYLNRTQGTTSGTTNQSRLLFSQAGPQSMSLQARNWLPGPCYRQQRLSKTANDNNNSNFPWTAASKYHLNGRDSLVNPGPAMASHKDDEEKFFPMHGNLIFGKEGTTASNAELDNVMITDEEEIRTTNPVATEQYGTV |
| | ANNLQSSNTAPTTRTVNDQGALPGMVWQDRDVYLQGPIWAKIPHTDGHFHPSPLMGGFGLKHPPPQIMIKNTPVPANPPTTFSPAKFASFITQYSTGQVSVEIEWELQKENSKRWNPEIQYTSNYNKSVNVDFTVDTNGVYSEPR |
| | PIGTRYLTRNL |
| 463 | > pP112 |
| | CTGCGCGCTCGCTCGCTCACTGAGGCCGCCCGGGCAAAGCCCGGGCGTCGGGCGACCTTTGGTCGCCCGGCCTCAGTGAGCGAGCGAGCGCGCAGAGAGGGAGTGGCCAACTCCATCACTAGGGGTTCCTGGAGGGGTGGAGTCG |
| | TGACGTGAATTACGTCATAGGGTTAGGGAGGTCGGCCGCTCTAGGAGTTAATTTTTAAAAAGCAGTCAAAAGTCCAAGTGCCCTTGCGAGCATTTACTCTCTCTGTTTGCTCTGGTTAATAATCTCAGGAGCACAAACATTCCTT |
| | ACTAGTTCTAGGAGTTAATTTTTAAAAAGCAGTCAAAAGTCCAAGTGCCCTTGCGAGCATTTACTCTCTCTGTTTGCTCTGGTTAATAATCTCAGGAGCACAAACATTCCTTACTAGTTCTAGAGCGGCCGCCAGTGTGCTGGAA |
| | TTCGGCTTTTTTAGGGCTGGAAGCTACCTTTGACATCATTTCCTCTGCGAATGCATGTATAATTTCTACAGAACCTATTAGAAAGGATCACCCAGCCTCTGCTTTTGTACAACTTTCCCTTAAAAAACTGCCAATCCCACTGCTG |
| | TTTGGCCCAATAGTGAGAACTTTTTCCTGCTGCCTCTTGGTGCTTTTGCCTATGGCCCCTATTCTGCCTGCTGAAGACACTCTTGCCAGCATGGACTTAAACCCCTCCAGCTCTGACAATCCTCTTTCTCTTTTGTTTTACATGA |
| | AGGGTCTGGCAGCCAAAGCAATCACTCAAAGTTCAAACCTTATCATTTTTTGCTTTGTTCCTCTTGGCCTTGGTTTTGTACATCAGCTTTGAAAATACCATCCCAGGGTTAATGCTGGGGTTAATTTATAACTGAGAGTGCTCTA |
| | GTTCTGCAATACAGGACATGCTATAAAAATGGAAAGATGTTGCTTTCTGAGAGATCAGCTTACATGTGGTACCGAGCTCGGATCCACTAGTAACGGCCGCCAGTGTGCTGGAATTCGGCTTTCCAGGCCATCTCCAACCATGGGA |
| | GTGAGGCACCCGCCCTGCTCCCACCGGCTCCTGGCCGTCTGCGCCCTCGTGTCCTTGGCAACCGCTGCACTCCTGGAGTTTGGGCTGAGCTGGGTCTTTCTGGTGGCCCTGCTGAAGGGAGTCCAGTGTGAGCAGCAGGGAGCCA |
| | GCAGACCAGGGCCCCGGGATGCCCAGGCACACCCCGGCCGTCCCAGAGCAGTGCCCACACAGTGCGACGTCCCCCCCAACAGCCGCTTCGATTGCGCCCCTGACAAGGCCATCACCCAGGAACAGTGCGAGGCCCGCGGCTGCTG |
| | CTACATCCCTGCAAAGCAGGGGCTGCAGGGAGCCCAGATGGGGCAGCCCTGGTGCTTCTTCCCACCCAGCTACCCCAGCTACAAGCTGGAGAACCTGAGCTCCTCTGAAATGGGCTACACGGCCACCCTGACCCGTACCACCCCC |
| | ACCTTCTTCCCCAAGGACATCCTGACCCTGCGGCTGGACGTGATGATGGAGACTGAGAACCGCCTCCACTTCACGATCAAAGATCCAGCTAACAGGCGCTACGAGGTGCCCTTGGAGACCCCGCGTGTCCACAGCCGGGCACCGT |
| | CCCCACTCTACAGCGTGGAGTTCTCCGAGGAGCCCTTCGGGGTGATCGTGCACCGGCAGCTGGACGGCCGCGTGCTGCTGAACACGACGGTGGCGCCCCTGTTCTTTGCGGACCAGTTCCTTCAGCTGTCCACCTCGCTGCCCTC |
| | GCAGTATATCACAGGCCTCGCCGAGCACCTCAGTCCCCTGATGCTCAGCACCAGCTGGACCAGGATCACCCTGTGGAACCGGGACCTTGCGCCCACGCCCGGTGCGAACCTCTACGGGTCTCACCCTTTCTACCTGGCGCTGGAG |
| | GACGGCGGGTCGGCACACGGGGTGTTCCTGCTAAACAGCAATGCCATGGATGTGGTCCTGCAGCCGAGCCCTGCCCTTAGCTGGAGGTCGACAGGTGGGATCCTGGATGTCTACATCTTCCTGGGCCCAGAGCCCAAGAGCGTGG |
| | TGCAGCAGTACCTGGACGTTGTGGGATACCCGTTCATGCCGCCATACTGGGGCCTGGGCTTCCACCTGTGCCGCTGGGGCTACTCCTCCACCGCTATCACCCGCCAGGTGGTGGAGAACATGACCAGGGCCCACTTCCCCCTGGA |
| | CGTCCAATGGAACGACCTGGACTACATGGACTCCCGGAGGGACTTCACGTTCAACAAGGATGGCTTCCGGGACTTCCCGGCCATGGTGCAGGAGCTGCACCAGGGCGGCCGGCGCTACATGATGATCGTGGATCCTGCCATCAGC |
| | AGCTCGGGCCCTGCCGGGAGCTACAGGCCCTACGACGAGGGTCTGCGGAGGGGGGTTTTCATCACCAACGAGACCGGCCAGCCGCTGATTGGGAAGGTATGGCCCGGGTCCACTGCCTTCCCCGACTTCACCAACCCCACAGCCC |
| | TGGCCTGGTGGGAGGACATGGTGGCTGAGTTCCATGACCAGGTGCCCTTCGACGGCATGTGGATTGACATGAACGAGCCTTCCAACTTCATCAGAGGCTCTGAGGACGGCTGCCCCAACAATGAGCTGGAGAACCCACCCTACGT |
| | GCCTGGGGTGGTTGGGGGGACCCTCCAGGCGGCCACCATCTGTGCCTCCAGCCACCAGTTTCTCTCCACACACTACAACCTGCACAACCTCTACGGCCTGACCGAAGCCATCGCCTCCCACAGGGCGCTGGTGAAGGCTCGGGGG |
| | ACACGCCCATTTGTGATCTCCCGCTCGACCTTTGCTGGCCACGGCCGATACGCCGGCCACTGGACGGGGGACGTGTGGAGCTCCTGGGAGCAGCTCGCCTCCTCCGTGCCAGAAATCCTGCAGTTTAACCTGCTGGGGGTGCCTC |
| | TGGTCGGGGCCGACGTCTGCGGCTTCCTGGGCAACACCTCAGAGGAGCTGTGTGTGCGCTGGACCCAGCTGGGGGCCTTCTACCCCTTCATGCGGAACCACAACAGCCTGCTCAGTCTGCCCCAGGAGCCGTACAGCTTCAGCGA |
| | GCCGGCCCAGCAGGCCATGAGGAAGGCCCTCACCCTGCGCTACGCACTCCTCCCCCACCTCTACACACTGTTCCACCAGGCCCACGTCGCGGGGGAGACCGTGGCCCGGCCCCTCTTCCTGGAGTTCCCCAAGGACTCTAGCACC |
| | TGGACTGTGGACCACCAGCTCCTGTGGGGGGAGGCCCTGCTCATCACCCCAGTGCTCCAGGCCGGGAAGGCCGAAGTGACTGGCTACTTCCCCTTGGGCACATGGTACGACCTGCAGACGGTGCCAATAGAGGCCCTTGGCAGCC |
| | TCCCACCCCCACCTGCAGCTCCCCGTGAGCCAGCCATCCACAGCGAGGGGCAGTGGGTGACGCTGCCGGCCCCCCTGGACACCATCAACGTCCACCTCCGGGCTGGGTACATCATCCCCCTGCAGGGCCCTGGCCTCACAACCAC |
| | AGAGTCCCGCCAGCAGCCCATGGCCCTGGCTGTGGCCCTGACCAAGGGTGGAGAGGCCCGAGGGGAGCTGTTCTGGGACGATGGAGAGAGCCTGGAAGTGCTGGAGCGAGGGGCCTACACACAGGTCATCTTCCTGGCCAGGAAT |
| | AACACGATCGTGAATGAGCTGGTACGTGTGACCAGTGAGGGAGCTGGCCTGCAGCTGCAGAAGGTGACTGTCCTGGGCGTGGCCACGGCGCCCCAGCAGGTCCTCTCCAACGGTGTCCCTGTCTCCAACTTCACCTACAGCCCCG |
| | ACACCAAGGTCCTGGACATCTGTGTCTCGCTGTTGATGGGAGAGCAGTTTCTCGTCAGCTGGTGTTAGCCGGGCGGAGTGTGTTAGTCTCTCCAGAGGGAGGCTGGTTCCCCAGGGAAGCAGAGCCTGTGTGCGGGCAGCAGCTG |
| | TGTGCGGGCCTGGGGGTTGTTAAGTGCAATTATTTTTAATAAAAGGGGCATTTGGAAAAAAAAAAAAAAGGTAGCAGTCGACAGATGAATTCTGCAGATCTGTGGCTTCTAGCTGCCCGGGTGGCATCCCTGTGACCCCTCCCCA |
| | GTGCCTCTCCTGGCCCTGGAAGTTGCCACTCCAGTGCCCACCAGCCTTGTCCTAATAAAATTAAGTTGCATCATTTTGTCTGACTAGGTGTCCTTCTATAATATTATGGGGTGGAGGGGGGTGGTATGGAGCAAGGGGCAAGTTG |
| | GGAAGACAACCTGAGTTGTTGGGATTCCAGGCATCGAGTAGATAAGTAGCATGGCGGGTTAATCATTAACTACAAGGAACCCCTAGTGATGGAGTTGGCCACTCCCTCTCTGCGCGCTCGCTCGCTCACTGAGGCCGGGCGACCA |
| | AAGGTCGCCCGACGCCCGGGCTTTGCCCGGGCGGCCTCAGTGAGCGAGCGAGCGCGCAGAGAGGGACAGATCCGAAGCCGGGCAAATCAGCGCCTGGCAGCAGTGGCGTCTGGCGGAAAACCTCAGTGTGACGCTCCCCGCCGCG |
| | TCCCACGCTTGTTCCCGGATCTGACCACCAGCGAAATCCGATTTTTGCACCGAGCTGGGTAATAAGCGTTGGCAATTTAACCGCCAGTCAGGCTTTCTTTCACAGTGTGGATTGGCGATAAAAAACAACTGCTGACGCCGCTGCG |
| | CGATCAGTTCACCCGTTCACCGCTGGATAACGACTTGGCGTAAGTGAAGCGACCCGTAAGACCCTAACGCCTGGGTCGAACGCTGGAAGGCGGCGGGCCAAACCAGGCCGAAGCAGCGTTGTTGCAGTTCACGGCAGATACACTT |
| | GCTGTTGCGGTGCTGATTACGACCGCTCACTCGTGGCAGCAACAGGGGAAAACCTTATTTATCAGCCGGAAAACCTACCGGATTGTTGGTAGTGGTCAATAGGCGATTACCGTTGTGTTGAAGTGGCGAGCGATACACCGCTTCC |
| | GGCGCGGATTGGCCTGAACTGCCAACTGGCGCAGGTAGCAGAGCGGGTAAACTGGCTCGGATTAGGGCCGCAAGAAAACTATCCCGACCGCCTTACTGCCGCCTGTTTTGACCGCTGGGATCTGCCAAGTCAGACAGTATAGCCC |
| | GTACGTCTTCCCGAGCGAAAACGGTCTGCGCTGCGGGACGCGCGAATTGAATTTGGCCCACACCAGTGGCGCGGCGACTTCCAGTTCAATATCAGCCGCTACAGTGAACAGCAACTGTTGGAAACCAGCCTTCGCCAACTGCTGC |
| | ACGCGGAAGAAGGCACTGGCTGAATATCGACGGTTTCCAGTTGGGGATTGGTGGCGACGACTCCTGGAGCCCGTCAGTATCGGCGGACTTCCAACTGAGCGCCGGTCGCTACCTTACCAGTTGGTCTGGTGTCAAAAAGCGTCCG |
| | CTTGAGTCTAGCGATCGCGCGCAGATCTGTCATGTGAGCAAAAGGCCAGCAAAAGGCCAGGAACCGTAAAAAGGCCGCGTTGCTGGCGTTTTTCCATAGGCTCCGCCCCCCTGACGAGCATCACAAAAATCGACGCTCAAGTCAG |
| | AGGTGGCGAAACCCGACAGGACTATAAAGATACCAGGCGTTTCCCCCTGGAAGCTCCCTCGTGCGCTCTCCTGTTCCGACCCTGCCGCTTACCGGATACCTGTCCGCCTTTCTCCCTTCGGGAAGCGTGGCGCTTTCTCATAGCT |
| | CACGCTGTAGGTATCTCAGTTCGGTGTAGGTCGTTCGCTCCAAGCTGGGCTGTGTGCACGAACCCCCCGTTCAGCCCGACCGCTGCGCCTTATCCGGTAACTATCGTCTTGAGTCCAACCCGGTAAGACACGACTTATCGCCACT |
| | GGCAGCAGCCACTGGTAACAGGATTAGCAGAGCGAGGTATGTAGGCGGTGCTACAGAGTTCTTGAAGTGGTGGCCTAACTACGGCTACACTAGAAGAACAGTATTTGGTATCTGCGCTCTGCTGAAGCCAGTTACCTTCGGAAAA |
| | AGAGTTGGTAGCTCTTGATCCGGCAAACAAACCACCGCTGGTAGCGGTGGTTTTTTTGTTTGCAAGCAGCAGATTACGCGCAGAAAAAAAGGATCTCAAGAAGATCCTTTGATCTTTTCTACGGGGTCTGACGCTCAGTGGAACG |
| | AAAACTCACGTTAAGGGATTTTGGTCATGAGATTATCAAAAAGGATCTTCACCTAGATCCTTTTCACGTAGAAAGCCAGTCCGCAGAAACGGTGCTGACCCCGGATGAATGTCAGCTACTGGGCTATCTGGACAAGGGAAAACGC |
| | AAGCGCAAAGAGAAAGCAGGTAGCTTGCAGTGGGCTTACATGGCGATAGCTAGACTGGGCGGTTTTATGGACAGCAAGCGAACCGGAATTGCCAGCTGGGGCGCCCTCTGGTAAGGTTGGGAAGCCCTGCAAAGTAAACTGGATG |
| | GCTTTCTTGCCGCCAAGGATCTGATGGCGCAGGGGATCAAGATCTGATCAAGAGACAGGATGAGGATCGTTTCGCATGATTGAACAAGATGGATTGCACGCAGGTTCTCCGGCCGCTTGGGTGGAGAGGCTATTCGGCTATGACT |
| | GGGCACAACAGACAATCGGCTGCTCTGATGCCGCCGTGTTCCGGCTGTCAGCGCAGGGGCGCCCGGTTCTTTTTGTCAAGACCGACCTGTCCGGTGCCCTGAATGAACTGCAAGACGAGGCAGCGCGGCTATCGTGGCTGGCCAC |
| | GACGGGCGTTCCTTGCGCAGCTGTGCTCGACGTTGTCACTGAAGCGGGAAGGGACTGGCTGCTATTGGGCGAAGTGCCGGGGCAGGATCTCCTGTCATCTCACCTTGCTCCTGCCGAGAAAGTATCCATCATGGCTGATGCAATG |
| | CGGCGGCTGCATACGCTTGATCCGGCTACCTGCCCATTCGACCACCAAGCGAAACATCGCATCGAGCGAGCACGTACTCGGATGGAAGCCGGTCTTGTCGATCAGGATGATCTGGACGAAGAGCATCAGGGGCTCGCGCCAGCCG |
| | AACTGTTCGCCAGGCTCAAGGCGAGCATGCCCGACGGCGAGGATCTCGTCGTGACCCATGGCGATGCCTGCTTGCCGAATATCATGGTGGAAAATGGCCGCTTTTCTGGATTCATCGACTGTGGCCGGCTGGGTGTGGCGGACCG |
| | CTATCAGGACATAGCGTTGGCTACCCGTGATATTGCTGAAGAGCTTGGCGGCGAATGGGCTGACCGCTTCCTCGTGCTTTACGGTATCGCCGCTCCCGATTCGCAGCGCATCGCCTTCTATCGCCTTCTTGACGAGTTCTTCTGA |
| | ATTTAAAGCCCAATACGCAAACCGCCTCTCCCCGCGCGTTGGCCGAAGCGGTTCAATATTTTGTTAAAATTCGCGTTAAATTTTTGTTAAATCAGCTATTTTTTAACCAATAGGCCGAAATCGGCAAAATCCCTTGTAAATCAAA |
| | AGAATAGACCGAGATAGGGTTGAGTGTTGTTCCAGTTTGGAACAAGAGTCCACTATTAAAGAACGTGGACTCCAACGTCAAAGGGCGAAAAACCGTCTATCAGGGCGTTGGCCCACTACGTGAACCTTCACCCTAATCAAGTTTT |
| | TTGGGGTCGAGGTGCCGTAAAGCACTAAATCGGAACCCTAAAGGGAGCCCCCGATTTAGAGCTTGACGGGGAAACCGGCGAACGTGGCGAGAAAGGAAGGGAAGAAAGCGAAAGGAGCGGGCGCTAGGGCGCTGGCAAGTGTAGC |
| | GGTCACGCTGCGCGTAACCACCACACCCGCCGCGCTAAGCGCCGCTACAGGGCGCGTCCCTTCGCCTTCAGGCTGCGTCGAGTACTGTACTGTGAGCCAGAGTTGCCCGGCGCTCTCCGGCTGCGGTAGTTCAGGCAGTTCAATC |
| | AACTGTTTACCTTGTGGAGCGACTCCAGAGGCACTTCACCGCTTGCCAGCGGCTTACGATCCAGCGCCACGATCCAGTGCAGGAGATCGTTATCGCTATACGGAACAGGTATTCGCTGGTCACTTCGATAAGGTTTGCCCGGATA |
| | AACGGAACTGGAAAAACTGCTGCTGGTGTTTTGCTTCCGTCAGTGCTGGATCGGCGTGCGGTCGGCAAAGACCAGACCGTTCTAACAGAACTGGCGATTGTTCGGCGTATCGCCAAAATCACCGCCGTAAGCCGACCACGGGTTG |
| | CCGTTTTCAGCAGGATTTAATCAGCGACTGATCCACCCAGTCCCAGACGAAGCCGCCCTGTAAACGGGGATACTGACGAAACGCCTGCCAGTATTTAGCGAAACCGCCAAGACTGTTACCCAAGCGTGGGCGTATTCGCAAAGGA |
| | TCAGCGGGCGCGTCTCTCCAGGTAGCGAAAGCCTTTTTTGATCGACCTTTCGGCACAGCCGGGAAGGGCTGGTCTTCAACCACGCGCGCGTACAACGGGCAAATAATATCGGTGGCCGTGGTGTCGGCTCCGCCGCCTTCAACTG |
| | CACCGGGCGGGAAGGATCGACAGATTTGATCCAGCGATACAGCGCGTCGTGATTAGCGCCGTGGCCTGATTCAATTCCCCAGCGACCAGTAGATCACACTCGGGTGATTACGATTGCGCTGCACCAGTCGCGTTACGGTTCGCTC |
| | TTCGCCGGTAGCCAGCGCGGATCACGGTCAGACGATTCGTTGGCACGATCCGTGGGTTTCAATACTGGCTTCAAACCACCACTAACAGGCCGTAGCGGTCGCACAGCGTGTACCACAGCGGTTGGTTCGGATAATCGAACAGCGC |
| | ACGGCGTTAAAGTTGTTCTGCTTCAACAGCAGGATATTCTGCACCTTCGTCTGCTCTTCCTAACCTGACCAAGCAGAGGATCTGCTCGTGACGGTTAATCCTCGAATCAGCAACGGCTTGCCGTTCAGCAGCAGCAGACCAAGTT |
| | CAATCCGCACCTCGCGGAAACCGACAACGCAGGCTTCTGCTTCAATCAGCGTGCCGTCGGCGGTGTGCAGTTCAACCACCGCACGATAGAGATTCGGGATTTCGGCGCTCCACAGTTTCGGGTTTTCGACGTTCAGACGTAGTGT |
| | GACGCGATCTGCAAACCACCACGCTCAACGATAATTTCACCGCCGAAAGGCGCGGTGCCGCTGGCGACCTGCGTTTCACCCTGCCAGAAAGAAACTGTTACCCGTAGGTAGTCACGCAACTCGCCGCACACTGAACTTCAGCCTC |
| | CAGTACAGCGCGGCTGAAATCGTCTTAAAGCGAGTGGCAACTGGAAATCGCTGATTTGTGTAGTCGGTTTAGCAGCAACGAGACTTCACGGAAAATCCGCTAATCCGCCACAGATCCTGATCTTCCAGATAACTGCCGTCACTCC |
| | AACGCAGCACCTTCACCGCGAGGCGGTTTTCTCCGGCGCGTAAAAATCGCTCAGGTCAAATTCAGACGGCAAACGACTGTCCTGGCCGTAACCGACCCAGCGCCCGTTGCACCACAGATTGAAACGCCGAGTTTACGCCTCAAAA |
| | ATAATTCGCGTCTGGCCTTCCTGTAGCCAGCTTTCACAACTATAATAGTGAGCGAGTAACAACCCGTCGGATTCTCCGTGGGAACAAACGGCGGATTGACCGTATAGGGATAGGTTACGTTGGTGTAGTAGGGCGCTCCGTAACC |
| | GTGCTACTGCCAGTTTGAGGGGACGACGACAGTATCGGCCTCAGGAAGATCGCACTCCAGCCAGCTTTCCGGCACCGCTTCTGGTACTGGAAACCAGGCAAAGCGCCTATCGCCTATCAGGCTGCACAACTGTTGGGAAGGGCGA |
| | TCTGTGCGGGCCTCTTCGCTATTACGCCAGCTTGCGAAAGGGGGTAGTGCTGCAAGGCGATTAAGTTGGGTAACGCCAGGGTTTTCCCAGTCACGACGTGGATCTGGGCCACTCCCTCT |
| 464 | >pP113 |
| | CTGCGCGCTCGCTCGCTCACTGAGGCCGCCCGGGCAAAGCCCGGGCGTCGGGCGACCTTTGGTCGCCCGGCCTCAGTGAGCGAGCGAGCGCGCAGAGAGGGAGTGGCCAACTCCATCACTAGGGGTTCCTGGAGGGGTGGAGTCG |
| | TGACGTGAATTACGTCATAGGGTTAGGGAGGTCGGCCGCTCTAGGAGTTAATTTTTAAAAAGCAGTCAAAAGTCCAAGTGCCCTTGCGAGCATTTACTCTCTCTGTTTGCTCTGGTTAATAATCTCAGGAGCACAAACATTCCTT |
| | ACTAGTTCTAGGAGTTAATTTTTAAAAAGCAGTCAAAAGTCCAAGTGCCCTTGCGAGCATTTACTCTCTCTGTTTGCTCTGGTTAATAATCTCAGGAGCACAAACATTCCTTACTAGTTCTAGAGCGGCCGCCAGTGTGCTGGAA |
| | TTCGGCTTTTTTAGGGCTGGAAGCTACCTTTGACATCATTTCCTCTGCGAATGCATGTATAATTTCTACAGAACCTATTAGAAAGGATCACCCAGCCTCTGCTTTTGTACAACTTTCCCTTAAAAAACTGCCAATCCCACTGCTG |
| | TTTGGCCCAATAGTGAGAACTTTTTCCTGCTGCCTCTTGGTGCTTTTGCCTATGGCCCCTATTCTGCCTGCTGAAGACACTCTTGCCAGCATGGACTTAAACCCCTCCAGCTCTGACAATCCTCTTTCTCTTTTGTTTTACATGA |
| | AGGGTCTGGCAGCCAAAGCAATCACTCAAAGTTCAAACCTTATCATTTTTTGCTTTGTTCCTCTTGGCCTTGGTTTTGTACATCAGCTTTGAAAATACCATCCCAGGGTTAATGCTGGGGTTAATTTATAACTGAGAGTGCTCTA |
| | GTTCTGCAATACAGGACATGCTATAAAAATGGAAAGATGTTGCTTTCTGAGAGATCAGCTTACATGTGGTACCGAGCTCGGATCCACTAGTAACGGCCGCCAGTGTGCTGGAATTCGGCTTTCCAGGCCATCTCCAACCATGGGA |
| | GTGAGGCACCCGCCCTGCTCCCACCGGCTCCTGGCCGTCTGCGCCCTCGTGTCCTTGGCAACCGCTGCACTCCTGGAGTTTGGGCTGAGCTGGGTCTTTCTGGTGGCCCTGCTGAAGGGAGTCCAGTGTGAGCAGCAGGGAGCCA |
| | GCAGACCAGGGCCCCGGGATGCCCAGGCACACCCCGGCCGTCCCAGAGCAGTGCCCACACAGTGCGACGTCCCCCCCAACAGCCGCTTCGATTGCGCCCCTGACAAGGCCATCACCCAGGAACAGTGCGAGGCCCGCGGCTGCTG |
| | CTACATCCCTGCAAAGCAGGGGCTGCAGGGAGCCCAGATGGGGCAGCCCTGGTGCTTCTTCCCACCCAGCTACCCCAGCTACAAGCTGGAGAACCTGAGCTCCTCTGAAATGGGCTACACGGCCACCCTGACCCGTACCACCCCC |
| | ACCTTCTTCCCCAAGGACATCCTGACCCTGCGGCTGGACGTGATGATGGAGACTGAGAACCGCCTCCACTTCACGATCAAAGATCCAGCTAACAGGCGCTACGAGGTGCCCTTGGAGACCCCGCGTGTCCACAGCCGGGCACCGT |
| | CCCCACTCTACAGCGTGGAGTTCTCCGAGGAGCCCTTCGGGGTGATCGTGCACCGGCAGCTGGACGGCCGCGTGCTGCTGAACACGACGGTGGCGCCCCTGTTCTTTGCGGACCAGTTCCTTCAGCTGTCCACCTCGCTGCCCTC |
| | GCAGTATATCACAGGCCTCGCCGAGCACCTCAGTCCCCTGATGCTCAGCACCAGCTGGACCAGGATCACCCTGTGGAACCGGGACCTTGCGCCCACGCCCGGTGCGAACCTCTACGGGTCTCACCCTTTCTACCTGGCGCTGGAG |
| | GACGGCGGGTCGGCACACGGGGTGTTCCTGCTAAACAGCAATGCCATGGATGTGGTCCTGCAGCCGAGCCCTGCCCTTAGCTGGAGGTCGACAGGTGGGATCCTGGATGTCTACATCTTCCTGGGCCCAGAGCCCAAGAGCGTGG |
| | TGCAGCAGTACCTGGACGTTGTGGGATACCCGTTCATGCCGCCATACTGGGGCCTGGGCTTCCACCTGTGCCGCTGGGGCTACTCCTCCACCGCTATCACCCGCCAGGTGGTGGAGAACATGACCAGGGCCCACTTCCCCCTGGA |
| | CGTCCAATGGAACGACCTGGACTACATGGACTCCCGGAGGGACTTCACGTTCAACAAGGATGGCTTCCGGGACTTCCCGGCCATGGTGCAGGAGCTGCACCAGGGCGGCCGGCGCTACATGATGATCGTGGATCCTGCCATCAGC |
| | AGCTCGGGCCCTGCCGGGAGCTACAGGCCCTACGACGAGGGTCTGCGGAGGGGGGTTTTCATCACCAACGAGACCGGCCAGCCGCTGATTGGGAAGGTATGGCCCGGGTCCACTGCCTTCCCCGACTTCACCAACCCCACAGCCC |
| | TGGCCTGGTGGGAGGACATGGTGGCTGAGTTCCATGACCAGGTGCCCTTCGACGGCATGTGGATTGACATGAACGAGCCTTCCAACTTCATCAGAGGCTCTGAGGACGGCTGCCCCAACAATGAGCTGGAGAACCCACCCTACGT |
| | GCCTGGGGTGGTTGGGGGGACCCTCCAGGCGGCCACCATCTGTGCCTCCAGCCACCAGTTTCTCTCCACACACTACAACCTGCACAACCTCTACGGCCTGACCGAAGCCATCGCCTCCCACAGGGCGCTGGTGAAGGCTCGGGGG |
| | ACACGCCCATTTGTGATCTCCCGCTCGACCTTTGCTGGCCACGGCCGATACGCCGGCCACTGGACGGGGGACGTGTGGAGCTCCTGGGAGCAGCTCGCCTCCTCCGTGCCAGAAATCCTGCAGTTTAACCTGCTGGGGGTGCCTC |
| | TGGTCGGGGCCGACGTCTGCGGCTTCCTGGGCAACACCTCAGAGGAGCTGTGTGTGCGCTGGACCCAGCTGGGGGCCTTCTACCCCTTCATGCGGAACCACAACAGCCTGCTCAGTCTGCCCCAGGAGCCGTACAGCTTCAGCGA |
| | GCCGGCCCAGCAGGCCATGAGGAAGGCCCTCACCCTGCGCTACGCACTCCTCCCCCACCTCTACACACTGTTCCACCAGGCCCACGTCGCGGGGGAGACCGTGGCCCGGCCCCTCTTCCTGGAGTTCCCCAAGGACTCTAGCACC |
| | TGGACTGTGGACCACCAGCTCCTGTGGGGGGAGGCCCTGCTCATCACCCCAGTGCTCCAGGCCGGGAAGGCCGAAGTGACTGGCTACTTCCCCTTGGGCACATGGTACGACCTGCAGACGGTGCCAATAGAGGCCCTTGGCAGCC |
| | TCCCACCCCCACCTGCAGCTCCCCGTGAGCCAGCCATCCACAGCGAGGGGCAGTGGGTGACGCTGCCGGCCCCCCTGGACACCATCAACGTCCACCTCCGGGCTGGGTACATCATCCCCCTGCAGGGCCCTGGCCTCACAACCAC |
| | AGAGTCCCGCCAGCAGCCCATGGCCCTGGCTGTGGCCCTGACCAAGGGTGGAGAGGCCCGAGGGGAGCTGTTCTGGGACGATGGAGAGAGCCTGGAAGTGCTGGAGCGAGGGGCCTACACACAGGTCATCTTCCTGGCCAGGAAT |
| | AACACGATCGTGAATGAGCTGGTACGTGTGACCAGTGAGGGAGCTGGCCTGCAGCTGCAGAAGGTGACTGTCCTGGGCGTGGCCACGGCGCCCCAGCAGGTCCTCTCCAACGGTGTCCCTGTCTCCAACTTCACCTACAGCCCCG |
| | ACACCAAGGTCCTGGACATCTGTGTCTCGCTGTTGATGGGAGAGCAGTTTCTCGTCAGCTGGTGTTAGCCGGGCGGAGTGTGTTAGTCTCTCCAGAGGGAGGCTGGTTCCCCAGGGAAGCAGAGCCTGTGTGCGGGCAGCAGCTG |
| | TGTGCGGGCCTGGGGGTTGTTAAGTGCAATTATTTTTAATAAAAGGGGCATTTGGAAAAAAAAAAAAAAGGTAGCAGTCGACAGATGAATTCTGCAGATCTGTGGCTTCTAGCTGCCCGGGTGGCATCCCTGTGACCCCTCCCCA |
| | GTGCCTCTCCTGGCCCTGGAAGTTGCCACTCCAGTGCCCACCAGCCTTGTCCTAATAAAATTAAGTTGCATCATTTTGTCTGACTAGGTGTCCTTCTATAATATTATGGGGTGGAGGGGGGTGGTATGGAGCAAGGGGCAAGTTG |
| | GGAAGACAACCTGAGTTGTTGGGATTCCAGGCATCGAGTAGATAAGTAGCATGGCGGGTTAATCATTAACTACAAGGAACCCCTAGTGATGGAGTTGGCCACTCCCTCTCTGCGCGCTCGCTCGCTCACTGAGGCCGGGCGACCA |
| | AAGGTCGCCCGACGCCCGGGCTTTGCCCGGGCGGCCTCAGTGAGCGAGCGAGCGCGCAGAGAGGGACAGATCCGAAGCCGGGCAAATCAGCGCCTGGCAGCAGTGGCGTCTGGCGGAAAACCTCAGTGTGACGCTCCCCGCCGCG |
| | TCCCACGCTTGTTCCCGGATCTGACCACCAGCGAAATCCGATTTTTGCACCGAGCTGGGTAATAAGCGTTGGCAATTTAACCGCCAGTCAGGCTTTCTTTCACAGTGTGGATTGGCGATAAAAAACAACTGCTGACGCCGCTGCG |
| | CGATCAGTTCACCCGTTCACCGCTGGATAACGACTTGGCGTAAGTGAAGCGACCCGTAAGACCCTAACGCCTGGGTCGAACGCTGGAAGGCGGCGGGCCAAACCAGGCCGAAGCAGCGTTGTTGCAGTTCACGGCAGATACACTT |
| | GCTGTTGCGGTGCTGATTACGACCGCTCACTCGTGGCAGCAACAGGGGAAAACCTTATTTATCAGCCGGAAAACCTACCGGATTGTTGGTAGTGGTCAATAGGCGATTACCGTTGTGTTGAAGTGGCGAGCGATACACCGCTTCC |
| | GGCGCGGATTGGCCTGAACTGCCAACTGGCGCAGGTAGCAGAGCGGGTAAACTGGCTCGGATTAGGGCCGCAAGAAAACTATCCCGACCGCCTTACTGCCGCCTGTTTTGACCGCTGGGATCTGCCAAGTCAGACAGTATAGCCC |
| | GTACGTCTTCCCGAGCGAAAACGGTCTGCGCTGCGGGACGCGCGAATTGAATTTGGCCCACACCAGTGGCGCGGCGACTTCCAGTTCAATATCAGCCGCTACAGTGAACAGCAACTGTTGGAAACCAGCCTTCGCCAACTGCTGC |
| | ACGCGGAAGAAGGCACTGGCTGAATATCGACGGTTTCCAGTTGGGGATTGGTGGCGACGACTCCTGGAGCCCGTCAGTATCGGCGGACTTCCAACTGAGCGCCGGTCGCTACCTTACCAGTTGGTCTGGTGTCAAAAAGCGTCCG |
| | CTTGAGTCTAGCGATCGCGCGCAGATCTGTCATGTGAGCAAAAGGCCAGCAAAAGGCCAGGAACCGTAAAAAGGCCGCGTTGCTGGCGTTTTTCCATAGGCTCCGCCCCCCTGACGAGCATCACAAAAATCGACGCTCAAGTCAG |
| | AGGTGGCGAAACCCGACAGGACTATAAAGATACCAGGCGTTTCCCCCTGGAAGCTCCCTCGTGCGCTCTCCTGTTCCGACCCTGCCGCTTACCGGATACCTGTCCGCCTTTCTCCCTTCGGGAAGCGTGGCGCTTTCTCATAGCT |
| | CACGCTGTAGGTATCTCAGTTCGGTGTAGGTCGTTCGCTCCAAGCTGGGCTGTGTGCACGAACCCCCCGTTCAGCCCGACCGCTGCGCCTTATCCGGTAACTATCGTCTTGAGTCCAACCCGGTAAGACACGACTTATCGCCACT |
| | GGCAGCAGCCACTGGTAACAGGATTAGCAGAGCGAGGTATGTAGGCGGTGCTACAGAGTTCTTGAAGTGGTGGCCTAACTACGGCTACACTAGAAGAACAGTATTTGGTATCTGCGCTCTGCTGAAGCCAGTTACCTTCGGAAAA |
| | AGAGTTGGTAGCTCTTGATCCGGCAAACAAACCACCGCTGGTAGCGGTGGTTTTTTTGTTTGCAAGCAGCAGATTACGCGCAGAAAAAAAGGATCTCAAGAAGATCCTTTGATCTTTTCTACGGGGTCTGACGCTCAGTGGAACG |
| | AAAACTCACGTTAAGGGATTTTGGTCATGAGATTATCAAAAAGGATCTTCACCTAGATCCTTTTCACGTAGAAAGCCAGTCCGCAGAAACGGTGCTGACCCCGGATGAATGTCAGCTACTGGGCTATCTGGACAAGGGAAAACGC |
| | AAGCGCAAAGAGAAAGCAGGTAGCTTGCAGTGGGCTTACATGGCGATAGCTAGACTGGGCGGTTTTATGGACAGCAAGCGAACCGGAATTGCCAGCTGGGGCGCCCTCTGGTAAGGTTGGGAAGCCCTGCAAAGTAAACTGGATG |
| | GCTTTCTTGCCGCCAAGGATCTGATGGCGCAGGGGATCAAGATCTGATCAAGAGACAGGATGAGGATCGTTTCGCATGATTGAACAAGATGGATTGCACGCAGGTTCTCCGGCCGCTTGGGTGGAGAGGCTATTCGGCTATGACT |
| | GGGCACAACAGACAATCGGCTGCTCTGATGCCGCCGTGTTCCGGCTGTCAGCGCAGGGGCGCCCGGTTCTTTTTGTCAAGACCGACCTGTCCGGTGCCCTGAATGAACTGCAAGACGAGGCAGCGCGGCTATCGTGGCTGGCCAC |
| | GACGGGCGTTCCTTGCGCAGCTGTGCTCGACGTTGTCACTGAAGCGGGAAGGGACTGGCTGCTATTGGGCGAAGTGCCGGGGCAGGATCTCCTGTCATCTCACCTTGCTCCTGCCGAGAAAGTATCCATCATGGCTGATGCAATG |
| | CGGCGGCTGCATACGCTTGATCCGGCTACCTGCCCATTCGACCACCAAGCGAAACATCGCATCGAGCGAGCACGTACTCGGATGGAAGCCGGTCTTGTCGATCAGGATGATCTGGACGAAGAGCATCAGGGGCTCGCGCCAGCCG |
| | AACTGTTCGCCAGGCTCAAGGCGAGCATGCCCGACGGCGAGGATCTCGTCGTGACCCATGGCGATGCCTGCTTGCCGAATATCATGGTGGAAAATGGCCGCTTTTCTGGATTCATCGACTGTGGCCGGCTGGGTGTGGCGGACCG |
| | CTATCAGGACATAGCGTTGGCTACCCGTGATATTGCTGAAGAGCTTGGCGGCGAATGGGCTGACCGCTTCCTCGTGCTTTACGGTATCGCCGCTCCCGATTCGCAGCGCATCGCCTTCTATCGCCTTCTTGACGAGTTCTTCTGA |
| | ATTTAAAGCCCAATACGCAAACCGCCTCTCCCCGCGCGTTGGCCGAAGCGGTTCAATATTTTGTTAAAATTCGCGTTAAATTTTTGTTAAATCAGCTATTTTTTAACCAATAGGCCGAAATCGGCAAAATCCCTTGTAAATCAAA |
| | AGAATAGACCGAGATAGGGTTGAGTGTTGTTCCAGTTTGGAACAAGAGTCCACTATTAAAGAACGTGGACTCCAACGTCAAAGGGCGAAAAACCGTCTATCAGGGCGTTGGCCCACTACGTGAACCTTCACCCTAATCAAGTTTT |
| | TTGGGGTCGAGGTGCCGTAAAGCACTAAATCGGAACCCTAAAGGGAGCCCCCGATTTAGAGCTTGACGGGGAAACCGGCGAACGTGGCGAGAAAGGAAGGGAAGAAAGCGAAAGGAGCGGGCGCTAGGGCGCTGGCAAGTGTAGC |
| | GGTCACGCTGCGCGTAACCACCACACCCGCCGCGCTAAGCGCCGCTACAGGGCGCGTCCCTTCGCCTTCAGGCTGCGTCGAGTACTGTACTGTGAGCCAGAGTTGCCCGGCGCTCTCCGGCTGCGGTAGTTCAGGCAGTTCAATC |
| | AACTGTTTACCTTGTGGAGCGACTCCAGAGGCACTTCACCGCTTGCCAGCGGCTTACGATCCAGCGCCACGATCCAGTGCAGGAGATCGTTATCGCTATACGGAACAGGTATTCGCTGGTCACTTCGATAAGGTTTGCCCGGATA |
| | AACGGAACTGGAAAAACTGCTGCTGGTGTTTTGCTTCCGTCAGTGCTGGATCGGCGTGCGGTCGGCAAAGACCAGACCGTTCTAACAGAACTGGCGATTGTTCGGCGTATCGCCAAAATCACCGCCGTAAGCCGACCACGGGTTG |
| | CCGTTTTCAGCAGGATTTAATCAGCGACTGATCCACCCAGTCCCAGACGAAGCCGCCCTGTAAACGGGGATACTGACGAAACGCCTGCCAGTATTTAGCGAAACCGCCAAGACTGTTACCCAAGCGTGGGCGTATTCGCAAAGGA |
| | TCAGCGGGCGCGTCTCTCCAGGTAGCGAAAGCCTTTTTTGATCGACCTTTCGGCACAGCCGGGAAGGGCTGGTCTTCAACCACGCGCGCGTACAACGGGCAAATAATATCGGTGGCCGTGGTGTCGGCTCCGCCGCCTTCAACTG |
| | CACCGGGCGGGAAGGATCGACAGATTTGATCCAGCGATACAGCGCGTCGTGATTAGCGCCGTGGCCTGATTCAATTCCCCAGCGACCAGTAGATCACACTCGGGTGATTACGATTGCGCTGCACCAGTCGCGTTACGGTTCGCTC |
| | TTCGCCGGTAGCCAGCGCGGATCACGGTCAGACGATTCGTTGGCACGATCCGTGGGTTTCAATACTGGCTTCAAACCACCACTAACAGGCCGTAGCGGTCGCACAGCGTGTACCACAGCGGTTGGTTCGGATAATCGAACAGCGC |
| | ACGGCGTTAAAGTTGTTCTGCTTCAACAGCAGGATATTCTGCACCTTCGTCTGCTCTTCCTAACCTGACCAAGCAGAGGATCTGCTCGTGACGGTTAATCCTCGAATCAGCAACGGCTTGCCGTTCAGCAGCAGCAGACCAAGTT |
| | CAATCCGCACCTCGCGGAAACCGACAACGCAGGCTTCTGCTTCAATCAGCGTGCCGTCGGCGGTGTGCAGTTCAACCACCGCACGATAGAGATTCGGGATTTCGGCGCTCCACAGTTTCGGGTTTTCGACGTTCAGACGTAGTGT |
| | GACGCGATCTGCAAACCACCACGCTCAACGATAATTTCACCGCCGAAAGGCGCGGTGCCGCTGGCGACCTGCGTTTCACCCTGCCAGAAAGAAACTGTTACCCGTAGGTAGTCACGCAACTCGCCGCACACTGAACTTCAGCCTC |
| | CAGTACAGCGCGGCTGAAATCGTCTTAAAGCGAGTGGCAACTGGAAATCGCTGATTTGTGTAGTCGGTTTAGCAGCAACGAGACTTCACGGAAAATCCGCTAATCCGCCACAGATCCTGATCTTCCAGATAACTGCCGTCACTCC |
| | AACGCAGCACCTTCACCGCGAGGCGGTTTTCTCCGGCGCGTAAAAATCGCTCAGGTCAAATTCAGACGGCAAACGACTGTCCTGGCCGTAACCGACCCAGCGCCCGTTGCACCACAGATTGAAACGCCGAGTTTACGCCTCAAAA |
| | ATAATTCGCGTCTGGCCTTCCTGTAGCCAGCTTTCACAACTATAATAGTGAGCGAGTAACAACCCGTCGGATTCTCCGTGGGAACAAACGGCGGATTGACCGTATAGGGATAGGTTACGTTGGTGTAGTAGGGCGCTCCGTAACC |
| | GTGCTACTGCCAGTTTGAGGGGACGACGACAGTATCGGCCTCAGGAAGATCGCACTCCAGCCAGCTTTCCGGCACCGCTTCTGGTACTGGAAACCAGGCAAAGCGCCTATCGCCTATCAGGCTGCACAACTGTTGGGAAGGGCGA |
| | TCTGTGCGGGCCTCTTCGCTATTACGCCAGCTTGCGAAAGGGGGTAGTGCTGCAAGGCGATTAAGTTGGGTAACGCCAGGGTTTTCCCAGTCACGACGTGGATCTGGGCCACTCCCTCT |
| 465 | >pP110 |
| | CTGCGCGCTCGCTCGCTCACTGAGGCCGCCCGGGCAAAGCCCGGGCGTCGGGCGACCTTTGGTCGCCCGGCCTCAGTGAGCGAGCGAGCGCGCAGAGAGGGAGTGGCCAACTCCATCACTAGGGGTTCCTGGAGGGGTGG |
| | AGTCGTGACGTGAATTACGTCATAGGGTTAGGGAGGTCGGCCGCTCTAGGAGTTAATTTTTAAAAAGCAGTCAAAAGTCCAAGTGCCCTTGCGAGCATTTACTCTCTCTGTTTGCTCTGGTTAATAATCTCAGGAGCACA |
| | AACATTCCTTACTAGTTCTAGGAGTTAATTTTTAAAAAGCAGTCAAAAGTCCAAGTGCCCTTGCGAGCATTTACTCTCTCTGTTTGCTCTGGTTAATAATCTCAGGAGCACAAACATTCCTTACTAGTTCTAGAGCGGCC |
| | GCCAGTGTGCTGGAATTCGGCTTTTTTAGGGCTGGAAGCTACCTTTGACATCATTTCCTCTGCGAATGCATGTATAATTTCTACAGAACCTATTAGAAAGGATCACCCAGCCTCTGCTTTTGTACAACTTTCCCTTAAAA |
| | AACTGCCAATCCCACTGCTGTTTGGCCCAATAGTGAGAACTTTTTCCTGCTGCCTCTTGGTGCTTTTGCCTATGGCCCCTATTCTGCCTGCTGAAGACACTCTTGCCAGCATGGACTTAAACCCCTCCAGCTCTGACAAT |
| | CCTCTTTCTCTTTTGTTTTACATGAAGGGTCTGGCAGCCAAAGCAATCACTCAAAGTTCAAACCTTATCATTTTTTGCTTTGTTCCTCTTGGCCTTGGTTTTGTACATCAGCTTTGAAAATACCATCCCAGGGTTAATGC |
| | TGGGGTTAATTTATAACTGAGAGTGCTCTAGTTCTGCAATACAGGACATGCTATAAAAATGGAAAGATGTTGCTTTCTGAGAGATCAGCTTACATGTGGTACCGAGCTCGGATCCACTAGTAACGGCCGCCAGTGTGCTG |
| | GAATTCGGCTTTCCAGGCCATCTCCAACCATGGGAGTGAGGCACCCGCCCTGCTCCCACCGGCTCCTGGCCGTCTGCGCCCTCGTGTCCTTGGCAACCGCTGCACTCCTGGGGCAGCAGGGAGCCAGCAGACCAGGGCCC |
| | CGGGATGCCCAGGCACACCCCGGCCGTCCCAGAGCAGTGCCCACACAGTGCGACGTCCCCCCCAACAGCCGCTTCGATTGCGCCCCTGACAAGGCCATCACCCAGGAACAGTGCGAGGCCCGCGGCTGCTGCTACATCCC |
| | TGCAAAGCAGGGGCTGCAGGGAGCCCAGATGGGGCAGCCCTGGTGCTTCTTCCCACCCAGCTACCCCAGCTACAAGCTGGAGAACCTGAGCTCCTCTGAAATGGGCTACACGGCCACCCTGACCCGTACCACCCCCACCT |
| | TCTTCCCCAAGGACATCCTGACCCTGCGGCTGGACGTGATGATGGAGACTGAGAACCGCCTCCACTTCACGATCAAAGATCCAGCTAACAGGCGCTACGAGGTGCCCTTGGAGACCCCGCGTGTCCACAGCCGGGCACCG |
| | TCCCCACTCTACAGCGTGGAGTTCTCCGAGGAGCCCTTCGGGGTGATCGTGCACCGGCAGCTGGACGGCCGCGTGCTGCTGAACACGACGGTGGCGCCCCTGTTCTTTGCGGACCAGTTCCTTCAGCTGTCCACCTCGCT |
| | GCCCTCGCAGTATATCACAGGCCTCGCCGAGCACCTCAGTCCCCTGATGCTCAGCACCAGCTGGACCAGGATCACCCTGTGGAACCGGGACCTTGCGCCCACGCCCGGTGCGAACCTCTACGGGTCTCACCCTTTCTACC |
| | TGGCGCTGGAGGACGGCGGGTCGGCACACGGGGTGTTCCTGCTAAACAGCAATGCCATGGATGTGGTCCTGCAGCCGAGCCCTGCCCTTAGCTGGAGGTCGACAGGTGGGATCCTGGATGTCTACATCTTCCTGGGCCCA |
| | GAGCCCAAGAGCGTGGTGCAGCAGTACCTGGACGTTGTGGGATACCCGTTCATGCCGCCATACTGGGGCCTGGGCTTCCACCTGTGCCGCTGGGGCTACTCCTCCACCGCTATCACCCGCCAGGTGGTGGAGAACATGAC |
| | CAGGGCCCACTTCCCCCTGGACGTCCAATGGAACGACCTGGACTACATGGACTCCCGGAGGGACTTCACGTTCAACAAGGATGGCTTCCGGGACTTCCCGGCCATGGTGCAGGAGCTGCACCAGGGCGGCCGGCGCTACA |
| | TGATGATCGTGGATCCTGCCATCAGCAGCTCGGGCCCTGCCGGGAGCTACAGGCCCTACGACGAGGGTCTGCGGAGGGGGGTTTTCATCACCAACGAGACCGGCCAGCCGCTGATTGGGAAGGTATGGCCCGGGTCCACT |
| | GCCTTCCCCGACTTCACCAACCCCACAGCCCTGGCCTGGTGGGAGGACATGGTGGCTGAGTTCCATGACCAGGTGCCCTTCGACGGCATGTGGATTGACATGAACGAGCCTTCCAACTTCATCAGAGGCTCTGAGGACGG |
| | CTGCCCCAACAATGAGCTGGAGAACCCACCCTACGTGCCTGGGGTGGTTGGGGGGACCCTCCAGGCGGCCACCATCTGTGCCTCCAGCCACCAGTTTCTCTCCACACACTACAACCTGCACAACCTCTACGGCCTGACCG |
| | AAGCCATCGCCTCCCACAGGGCGCTGGTGAAGGCTCGGGGGACACGCCCATTTGTGATCTCCCGCTCGACCTTTGCTGGCCACGGCCGATACGCCGGCCACTGGACGGGGGACGTGTGGAGCTCCTGGGAGCAGCTCGCC |
| | TCCTCCGTGCCAGAAATCCTGCAGTTTAACCTGCTGGGGGTGCCTCTGGTCGGGGCCGACGTCTGCGGCTTCCTGGGCAACACCTCAGAGGAGCTGTGTGTGCGCTGGACCCAGCTGGGGGCCTTCTACCCCTTCATGCG |
| | GAACCACAACAGCCTGCTCAGTCTGCCCCAGGAGCCGTACAGCTTCAGCGAGCCGGCCCAGCAGGCCATGAGGAAGGCCCTCACCCTGCGCTACGCACTCCTCCCCCACCTCTACACACTGTTCCACCAGGCCCACGTCG |
| | CGGGGGAGACCGTGGCCCGGCCCCTCTTCCTGGAGTTCCCCAAGGACTCTAGCACCTGGACTGTGGACCACCAGCTCCTGTGGGGGGAGGCCCTGCTCATCACCCCAGTGCTCCAGGCCGGGAAGGCCGAAGTGACTGGC |
| | TACTTCCCCTTGGGCACATGGTACGACCTGCAGACGGTGCCAATAGAGGCCCTTGGCAGCCTCCCACCCCCACCTGCAGCTCCCCGTGAGCCAGCCATCCACAGCGAGGGGCAGTGGGTGACGCTGCCGGCCCCCCTGGA |
| | CACCATCAACGTCCACCTCCGGGCTGGGTACATCATCCCCCTGCAGGGCCCTGGCCTCACAACCACAGAGTCCCGCCAGCAGCCCATGGCCCTGGCTGTGGCCCTGACCAAGGGTGGAGAGGCCCGAGGGGAGCTGTTCT |
| | GGGACGATGGAGAGAGCCTGGAAGTGCTGGAGCGAGGGGCCTACACACAGGTCATCTTCCTGGCCAGGAATAACACGATCGTGAATGAGCTGGTACGTGTGACCAGTGAGGGAGCTGGCCTGCAGCTGCAGAAGGTGACT |
| | GTCCTGGGCGTGGCCACGGCGCCCCAGCAGGTCCTCTCCAACGGTGTCCCTGTCTCCAACTTCACCTACAGCCCCGACACCAAGGTCCTGGACATCTGTGTCTCGCTGTTGATGGGAGAGCAGTTTCTCGTCAGCTGGTG |
| | TTAGCCGGGCGGAGTGTGTTAGTCTCTCCAGAGGGAGGCTGGTTCCCCAGGGAAGCAGAGCCTGTGTGCGGGCAGCAGCTGTGTGCGGGCCTGGGGGTTGTTAAGTGCAATTATTTTTAATAAAAGGGGCATTTGGAAAA |
| | AAAAAAAAAAGGTAGCAGTCGACAGATGAATTCTGCAGATCTGTGGCTTCTAGCTGCCCGGGTGGCATCCCTGTGACCCCTCCCCAGTGCCTCTCCTGGCCCTGGAAGTTGCCACTCCAGTGCCCACCAGCCTTGTCCTA |
| | ATAAAATTAAGTTGCATCATTTTGTCTGACTAGGTGTCCTTCTATAATATTATGGGGTGGAGGGGGGTGGTATGGAGCAAGGGGCAAGTTGGGAAGACAACCTGAGTTGTTGGGATTCCAGGCATCGAGTAGATAAGTAG |
| | CATGGCGGGTTAATCATTAACTACAAGGAACCCCTAGTGATGGAGTTGGCCACTCCCTCTCTGCGCGCTCGCTCGCTCACTGAGGCCGGGCGACCAAAGGTCGCCCGACGCCCGGGCTTTGCCCGGGCGGCCTCAGTGAG |
| | CGAGCGAGCGCGCAGAGAGGGACAGATCCGAAGCCGGGCAAATCAGCGCCTGGCAGCAGTGGCGTCTGGCGGAAAACCTCAGTGTGACGCTCCCCGCCGCGTCCCACGCTTGTTCCCGGATCTGACCACCAGCGAAATCC |
| | GATTTTTGCACCGAGCTGGGTAATAAGCGTTGGCAATTTAACCGCCAGTCAGGCTTTCTTTCACAGTGTGGATTGGCGATAAAAAACAACTGCTGACGCCGCTGCGCGATCAGTTCACCCGTTCACCGCTGGATAACGAC |
| | TTGGCGTAAGTGAAGCGACCCGTAAGACCCTAACGCCTGGGTCGAACGCTGGAAGGCGGCGGGCCAAACCAGGCCGAAGCAGCGTTGTTGCAGTTCACGGCAGATACACTTGCTGTTGCGGTGCTGATTACGACCGCTCA |
| | CTCGTGGCAGCAACAGGGGAAAACCTTATTTATCAGCCGGAAAACCTACCGGATTGTTGGTAGTGGTCAATAGGCGATTACCGTTGTGTTGAAGTGGCGAGCGATACACCGCTTCCGGCGCGGATTGGCCTGAACTGCCA |
| | ACTGGCGCAGGTAGCAGAGCGGGTAAACTGGCTCGGATTAGGGCCGCAAGAAAACTATCCCGACCGCCTTACTGCCGCCTGTTTTGACCGCTGGGATCTGCCAAGTCAGACAGTATAGCCCGTACGTCTTCCCGAGCGAA |
| | AACGGTCTGCGCTGCGGGACGCGCGAATTGAATTTGGCCCACACCAGTGGCGCGGCGACTTCCAGTTCAATATCAGCCGCTACAGTGAACAGCAACTGTTGGAAACCAGCCTTCGCCAACTGCTGCACGCGGAAGAAGGC |
| | ACTGGCTGAATATCGACGGTTTCCAGTTGGGGATTGGTGGCGACGACTCCTGGAGCCCGTCAGTATCGGCGGACTTCCAACTGAGCGCCGGTCGCTACCTTACCAGTTGGTCTGGTGTCAAAAAGCGTCCGCTTGAGTCT |
| | AGCGATCGCGCGCAGATCTGTCATGTGAGCAAAAGGCCAGCAAAAGGCCAGGAACCGTAAAAAGGCCGCGTTGCTGGCGTTTTTCCATAGGCTCCGCCCCCCTGACGAGCATCACAAAAATCGACGCTCAAGTCAGAGGT |
| | GGCGAAACCCGACAGGACTATAAAGATACCAGGCGTTTCCCCCTGGAAGCTCCCTCGTGCGCTCTCCTGTTCCGACCCTGCCGCTTACCGGATACCTGTCCGCCTTTCTCCCTTCGGGAAGCGTGGCGCTTTCTCATAGC |
| | TCACGCTGTAGGTATCTCAGTTCGGTGTAGGTCGTTCGCTCCAAGCTGGGCTGTGTGCACGAACCCCCCGTTCAGCCCGACCGCTGCGCCTTATCCGGTAACTATCGTCTTGAGTCCAACCCGGTAAGACACGACTTATC |
| | GCCACTGGCAGCAGCCACTGGTAACAGGATTAGCAGAGCGAGGTATGTAGGCGGTGCTACAGAGTTCTTGAAGTGGTGGCCTAACTACGGCTACACTAGAAGAACAGTATTTGGTATCTGCGCTCTGCTGAAGCCAGTTA |
| | CCTTCGGAAAAAGAGTTGGTAGCTCTTGATCCGGCAAACAAACCACCGCTGGTAGCGGTGGTTTTTTTGTTTGCAAGCAGCAGATTACGCGCAGAAAAAAAGGATCTCAAGAAGATCCTTTGATCTTTTCTACGGGGTCT |
| | GACGCTCAGTGGAACGAAAACTCACGTTAAGGGATTTTGGTCATGAGATTATCAAAAAGGATCTTCACCTAGATCCTTTTCACGTAGAAAGCCAGTCCGCAGAAACGGTGCTGACCCCGGATGAATGTCAGCTACTGGGC |
| | TATCTGGACAAGGGAAAACGCAAGCGCAAAGAGAAAGCAGGTAGCTTGCAGTGGGCTTACATGGCGATAGCTAGACTGGGCGGTTTTATGGACAGCAAGCGAACCGGAATTGCCAGCTGGGGCGCCCTCTGGTAAGGTTG |
| | GGAAGCCCTGCAAAGTAAACTGGATGGCTTTCTTGCCGCCAAGGATCTGATGGCGCAGGGGATCAAGATCTGATCAAGAGACAGGATGAGGATCGTTTCGCATGATTGAACAAGATGGATTGCACGCAGGTTCTCCGGCC |
| | GCTTGGGTGGAGAGGCTATTCGGCTATGACTGGGCACAACAGACAATCGGCTGCTCTGATGCCGCCGTGTTCCGGCTGTCAGCGCAGGGGCGCCCGGTTCTTTTTGTCAAGACCGACCTGTCCGGTGCCCTGAATGAACT |
| | GCAAGACGAGGCAGCGCGGCTATCGTGGCTGGCCACGACGGGCGTTCCTTGCGCAGCTGTGCTCGACGTTGTCACTGAAGCGGGAAGGGACTGGCTGCTATTGGGCGAAGTGCCGGGGCAGGATCTCCTGTCATCTCACC |
| | TTGCTCCTGCCGAGAAAGTATCCATCATGGCTGATGCAATGCGGCGGCTGCATACGCTTGATCCGGCTACCTGCCCATTCGACCACCAAGCGAAACATCGCATCGAGCGAGCACGTACTCGGATGGAAGCCGGTCTTGTC |
| | GATCAGGATGATCTGGACGAAGAGCATCAGGGGCTCGCGCCAGCCGAACTGTTCGCCAGGCTCAAGGCGAGCATGCCCGACGGCGAGGATCTCGTCGTGACCCATGGCGATGCCTGCTTGCCGAATATCATGGTGGAAAA |
| | TGGCCGCTTTTCTGGATTCATCGACTGTGGCCGGCTGGGTGTGGCGGACCGCTATCAGGACATAGCGTTGGCTACCCGTGATATTGCTGAAGAGCTTGGCGGCGAATGGGCTGACCGCTTCCTCGTGCTTTACGGTATCG |
| | CCGCTCCCGATTCGCAGCGCATCGCCTTCTATCGCCTTCTTGACGAGTTCTTCTGAATTTAAAGCCCAATACGCAAACCGCCTCTCCCCGCGCGTTGGCCGAAGCGGTTCAATATTTTGTTAAAATTCGCGTTAAATTTT |
| | TGTTAAATCAGCTATTTTTTAACCAATAGGCCGAAATCGGCAAAATCCCTTGTAAATCAAAAGAATAGACCGAGATAGGGTTGAGTGTTGTTCCAGTTTGGAACAAGAGTCCACTATTAAAGAACGTGGACTCCAACGTC |
| | AAAGGGCGAAAAACCGTCTATCAGGGCGTTGGCCCACTACGTGAACCTTCACCCTAATCAAGTTTTTTGGGGTCGAGGTGCCGTAAAGCACTAAATCGGAACCCTAAAGGGAGCCCCCGATTTAGAGCTTGACGGGGAAA |
| | CCGGCGAACGTGGCGAGAAAGGAAGGGAAGAAAGCGAAAGGAGCGGGCGCTAGGGCGCTGGCAAGTGTAGCGGTCACGCTGCGCGTAACCACCACACCCGCCGCGCTAAGCGCCGCTACAGGGCGCGTCCCTTCGCCTTC |
| | AGGCTGCGTCGAGTACTGTACTGTGAGCCAGAGTTGCCCGGCGCTCTCCGGCTGCGGTAGTTCAGGCAGTTCAATCAACTGTTTACCTTGTGGAGCGACTCCAGAGGCACTTCACCGCTTGCCAGCGGCTTACGATCCAG |
| | CGCCACGATCCAGTGCAGGAGATCGTTATCGCTATACGGAACAGGTATTCGCTGGTCACTTCGATAAGGTTTGCCCGGATAAACGGAACTGGAAAAACTGCTGCTGGTGTTTTGCTTCCGTCAGTGCTGGATCGGCGTGC |
| | GGTCGGCAAAGACCAGACCGTTCTAACAGAACTGGCGATTGTTCGGCGTATCGCCAAAATCACCGCCGTAAGCCGACCACGGGTTGCCGTTTTCAGCAGGATTTAATCAGCGACTGATCCACCCAGTCCCAGACGAAGCC |
| | GCCCTGTAAACGGGGATACTGACGAAACGCCTGCCAGTATTTAGCGAAACCGCCAAGACTGTTACCCAAGCGTGGGCGTATTCGCAAAGGATCAGCGGGCGCGTCTCTCCAGGTAGCGAAAGCCTTTTTTGATCGACCTT |
| | TCGGCACAGCCGGGAAGGGCTGGTCTTCAACCACGCGCGCGTACAACGGGCAAATAATATCGGTGGCCGTGGTGTCGGCTCCGCCGCCTTCAACTGCACCGGGCGGGAAGGATCGACAGATTTGATCCAGCGATACAGCG |
| | CGTCGTGATTAGCGCCGTGGCCTGATTCAATTCCCCAGCGACCAGTAGATCACACTCGGGTGATTACGATTGCGCTGCACCAGTCGCGTTACGGTTCGCTCTTCGCCGGTAGCCAGCGCGGATCACGGTCAGACGATTCG |
| | TTGGCACGATCCGTGGGTTTCAATACTGGCTTCAAACCACCACTAACAGGCCGTAGCGGTCGCACAGCGTGTACCACAGCGGTTGGTTCGGATAATCGAACAGCGCACGGCGTTAAAGTTGTTCTGCTTCAACAGCAGGA |
| | TATTCTGCACCTTCGTCTGCTCTTCCTAACCTGACCAAGCAGAGGATCTGCTCGTGACGGTTAATCCTCGAATCAGCAACGGCTTGCCGTTCAGCAGCAGCAGACCAAGTTCAATCCGCACCTCGCGGAAACCGACAACG |
| | CAGGCTTCTGCTTCAATCAGCGTGCCGTCGGCGGTGTGCAGTTCAACCACCGCACGATAGAGATTCGGGATTTCGGCGCTCCACAGTTTCGGGTTTTCGACGTTCAGACGTAGTGTGACGCGATCTGCAAACCACCACGC |
| | TCAACGATAATTTCACCGCCGAAAGGCGCGGTGCCGCTGGCGACCTGCGTTTCACCCTGCCAGAAAGAAACTGTTACCCGTAGGTAGTCACGCAACTCGCCGCACACTGAACTTCAGCCTCCAGTACAGCGCGGCTGAAA |
| | TCGTCTTAAAGCGAGTGGCAACTGGAAATCGCTGATTTGTGTAGTCGGTTTAGCAGCAACGAGACTTCACGGAAAATCCGCTAATCCGCCACAGATCCTGATCTTCCAGATAACTGCCGTCACTCCAACGCAGCACCTTC |
| | ACCGCGAGGCGGTTTTCTCCGGCGCGTAAAAATCGCTCAGGTCAAATTCAGACGGCAAACGACTGTCCTGGCCGTAACCGACCCAGCGCCCGTTGCACCACAGATTGAAACGCCGAGTTTACGCCTCAAAAATAATTCGC |
| | GTCTGGCCTTCCTGTAGCCAGCTTTCACAACTATAATAGTGAGCGAGTAACAACCCGTCGGATTCTCCGTGGGAACAAACGGCGGATTGACCGTATAGGGATAGGTTACGTTGGTGTAGTAGGGCGCTCCGTAACCGTGC |
| | TACTGCCAGTTTGAGGGGACGACGACAGTATCGGCCTCAGGAAGATCGCACTCCAGCCAGCTTTCCGGCACCGCTTCTGGTACTGGAAACCAGGCAAAGCGCCTATCGCCTATCAGGCTGCACAACTGTTGGGAAGGGCG |
| | ATCTGTGCGGGCCTCTTCGCTATTACGCCAGCTTGCGAAAGGGGGTAGTGCTGCAAGGCGATTAAGTTGGGTAACGCCAGGGTTTTCCCAGTCACGACGTGGATCTGGGCCACTCCCTCT |
| 466 | >pP111 |
| | CTGCGCGCTCGCTCGCTCACTGAGGCCGCCCGGGCAAAGCCCGGGCGTCGGGCGACCTTTGGTCGCCCGGCCTCAGTGAGCGAGCGAGCGCGCAGAGAGGGAGTGGCCAACTCCATCACTAGGGGTTCCTGGAGGGGTGG |
| | AGTCGTGACGTGAATTACGTCATAGGGTTAGGGAGGTCGGCCGCTCTAGGAGTTAATTTTTAAAAAGCAGTCAAAAGTCCAAGTGCCCTTGCGAGCATTTACTCTCTCTGTTTGCTCTGGTTAATAATCTCAGGAGCACA |
| | AACATTCCTTACTAGTTCTAGGAGTTAATTTTTAAAAAGCAGTCAAAAGTCCAAGTGCCCTTGCGAGCATTTACTCTCTCTGTTTGCTCTGGTTAATAATCTCAGGAGCACAAACATTCCTTACTAGTTCTAGAGCGGCC |
| | GCCAGTGTGCTGGAATTCGGCTTTTTTAGGGCTGGAAGCTACCTTTGACATCATTTCCTCTGCGAATGCATGTATAATTTCTACAGAACCTATTAGAAAGGATCACCCAGCCTCTGCTTTTGTACAACTTTCCCTTAAAA |
| | AACTGCCAATCCCACTGCTGTTTGGCCCAATAGTGAGAACTTTTTCCTGCTGCCTCTTGGTGCTTTTGCCTATGGCCCCTATTCTGCCTGCTGAAGACACTCTTGCCAGCATGGACTTAAACCCCTCCAGCTCTGACAAT |
| | CCTCTTTCTCTTTTGTTTTACATGAAGGGTCTGGCAGCCAAAGCAATCACTCAAAGTTCAAACCTTATCATTTTTTGCTTTGTTCCTCTTGGCCTTGGTTTTGTACATCAGCTTTGAAAATACCATCCCAGGGTTAATGC |
| | TGGGGTTAATTTATAACTGAGAGTGCTCTAGTTCTGCAATACAGGACATGCTATAAAAATGGAAAGATGTTGCTTTCTGAGAGATCAGCTTACATGTGGTACCGAGCTCGGATCCACTAGTAACGGCCGCCAGTGTGCTG |
| | GAATTCGGCTTTCCAGGCCATCTCCAACCATGGGAGTGAGGCACCCGCCCTGCTCCCACCGGCTCCTGGCCGTCTGCGCCCTCGTGTCCTTGGCAACCGCTCAGCAGGGAGCCAGCAGACCAGGGCCCCGGGATGCCCAG |
| | GCACACCCCGGCCGTCCCAGAGCAGTGCCCACACAGTGCGACGTCCCCCCCAACAGCCGCTTCGATTGCGCCCCTGACAAGGCCATCACCCAGGAACAGTGCGAGGCCCGCGGCTGCTGCTACATCCCTGCAAAGCAGGG |
| | GCTGCAGGGAGCCCAGATGGGGCAGCCCTGGTGCTTCTTCCCACCCAGCTACCCCAGCTACAAGCTGGAGAACCTGAGCTCCTCTGAAATGGGCTACACGGCCACCCTGACCCGTACCACCCCCACCTTCTTCCCCAAGG |
| | ACATCCTGACCCTGCGGCTGGACGTGATGATGGAGACTGAGAACCGCCTCCACTTCACGATCAAAGATCCAGCTAACAGGCGCTACGAGGTGCCCTTGGAGACCCCGCGTGTCCACAGCCGGGCACCGTCCCCACTCTAC |
| | AGCGTGGAGTTCTCCGAGGAGCCCTTCGGGGTGATCGTGCACCGGCAGCTGGACGGCCGCGTGCTGCTGAACACGACGGTGGCGCCCCTGTTCTTTGCGGACCAGTTCCTTCAGCTGTCCACCTCGCTGCCCTCGCAGTA |
| | TATCACAGGCCTCGCCGAGCACCTCAGTCCCCTGATGCTCAGCACCAGCTGGACCAGGATCACCCTGTGGAACCGGGACCTTGCGCCCACGCCCGGTGCGAACCTCTACGGGTCTCACCCTTTCTACCTGGCGCTGGAGG |
| | ACGGCGGGTCGGCACACGGGGTGTTCCTGCTAAACAGCAATGCCATGGATGTGGTCCTGCAGCCGAGCCCTGCCCTTAGCTGGAGGTCGACAGGTGGGATCCTGGATGTCTACATCTTCCTGGGCCCAGAGCCCAAGAGC |
| | GTGGTGCAGCAGTACCTGGACGTTGTGGGATACCCGTTCATGCCGCCATACTGGGGCCTGGGCTTCCACCTGTGCCGCTGGGGCTACTCCTCCACCGCTATCACCCGCCAGGTGGTGGAGAACATGACCAGGGCCCACTT |
| | CCCCCTGGACGTCCAATGGAACGACCTGGACTACATGGACTCCCGGAGGGACTTCACGTTCAACAAGGATGGCTTCCGGGACTTCCCGGCCATGGTGCAGGAGCTGCACCAGGGCGGCCGGCGCTACATGATGATCGTGG |
| | ATCCTGCCATCAGCAGCTCGGGCCCTGCCGGGAGCTACAGGCCCTACGACGAGGGTCTGCGGAGGGGGGTTTTCATCACCAACGAGACCGGCCAGCCGCTGATTGGGAAGGTATGGCCCGGGTCCACTGCCTTCCCCGAC |
| | TTCACCAACCCCACAGCCCTGGCCTGGTGGGAGGACATGGTGGCTGAGTTCCATGACCAGGTGCCCTTCGACGGCATGTGGATTGACATGAACGAGCCTTCCAACTTCATCAGAGGCTCTGAGGACGGCTGCCCCAACAA |
| | TGAGCTGGAGAACCCACCCTACGTGCCTGGGGTGGTTGGGGGGACCCTCCAGGCGGCCACCATCTGTGCCTCCAGCCACCAGTTTCTCTCCACACACTACAACCTGCACAACCTCTACGGCCTGACCGAAGCCATCGCCT |
| | CCCACAGGGCGCTGGTGAAGGCTCGGGGGACACGCCCATTTGTGATCTCCCGCTCGACCTTTGCTGGCCACGGCCGATACGCCGGCCACTGGACGGGGGACGTGTGGAGCTCCTGGGAGCAGCTCGCCTCCTCCGTGCCA |
| | GAAATCCTGCAGTTTAACCTGCTGGGGGTGCCTCTGGTCGGGGCCGACGTCTGCGGCTTCCTGGGCAACACCTCAGAGGAGCTGTGTGTGCGCTGGACCCAGCTGGGGGCCTTCTACCCCTTCATGCGGAACCACAACAG |
| | CCTGCTCAGTCTGCCCCAGGAGCCGTACAGCTTCAGCGAGCCGGCCCAGCAGGCCATGAGGAAGGCCCTCACCCTGCGCTACGCACTCCTCCCCCACCTCTACACACTGTTCCACCAGGCCCACGTCGCGGGGGAGACCG |
| | TGGCCCGGCCCCTCTTCCTGGAGTTCCCCAAGGACTCTAGCACCTGGACTGTGGACCACCAGCTCCTGTGGGGGGAGGCCCTGCTCATCACCCCAGTGCTCCAGGCCGGGAAGGCCGAAGTGACTGGCTACTTCCCCTTG |
| | GGCACATGGTACGACCTGCAGACGGTGCCAATAGAGGCCCTTGGCAGCCTCCCACCCCCACCTGCAGCTCCCCGTGAGCCAGCCATCCACAGCGAGGGGCAGTGGGTGACGCTGCCGGCCCCCCTGGACACCATCAACGT |
| | CCACCTCCGGGCTGGGTACATCATCCCCCTGCAGGGCCCTGGCCTCACAACCACAGAGTCCCGCCAGCAGCCCATGGCCCTGGCTGTGGCCCTGACCAAGGGTGGAGAGGCCCGAGGGGAGCTGTTCTGGGACGATGGAG |
| | AGAGCCTGGAAGTGCTGGAGCGAGGGGCCTACACACAGGTCATCTTCCTGGCCAGGAATAACACGATCGTGAATGAGCTGGTACGTGTGACCAGTGAGGGAGCTGGCCTGCAGCTGCAGAAGGTGACTGTCCTGGGCGTG |
| | GCCACGGCGCCCCAGCAGGTCCTCTCCAACGGTGTCCCTGTCTCCAACTTCACCTACAGCCCCGACACCAAGGTCCTGGACATCTGTGTCTCGCTGTTGATGGGAGAGCAGTTTCTCGTCAGCTGGTGTTAGCCGGGCGG |
| | AGTGTGTTAGTCTCTCCAGAGGGAGGCTGGTTCCCCAGGGAAGCAGAGCCTGTGTGCGGGCAGCAGCTGTGTGCGGGCCTGGGGGTTGTTAAGTGCAATTATTTTTAATAAAAGGGGCATTTGGAAAAAAAAAAAAAAGG |
| | TAGCAGTCGACAGATGAATTCTGCAGATCTGTGGCTTCTAGCTGCCCGGGTGGCATCCCTGTGACCCCTCCCCAGTGCCTCTCCTGGCCCTGGAAGTTGCCACTCCAGTGCCCACCAGCCTTGTCCTAATAAAATTAAGT |
| | TGCATCATTTTGTCTGACTAGGTGTCCTTCTATAATATTATGGGGTGGAGGGGGGTGGTATGGAGCAAGGGGCAAGTTGGGAAGACAACCTGAGTTGTTGGGATTCCAGGCATCGAGTAGATAAGTAGCATGGCGGGTTA |
| | ATCATTAACTACAAGGAACCCCTAGTGATGGAGTTGGCCACTCCCTCTCTGCGCGCTCGCTCGCTCACTGAGGCCGGGCGACCAAAGGTCGCCCGACGCCCGGGCTTTGCCCGGGCGGCCTCAGTGAGCGAGCGAGCGCG |
| | CAGAGAGGGACAGATCCGAAGCCGGGCAAATCAGCGCCTGGCAGCAGTGGCGTCTGGCGGAAAACCTCAGTGTGACGCTCCCCGCCGCGTCCCACGCTTGTTCCCGGATCTGACCACCAGCGAAATCCGATTTTTGCACC |
| | GAGCTGGGTAATAAGCGTTGGCAATTTAACCGCCAGTCAGGCTTTCTTTCACAGTGTGGATTGGCGATAAAAAACAACTGCTGACGCCGCTGCGCGATCAGTTCACCCGTTCACCGCTGGATAACGACTTGGCGTAAGTG |
| | AAGCGACCCGTAAGACCCTAACGCCTGGGTCGAACGCTGGAAGGCGGCGGGCCAAACCAGGCCGAAGCAGCGTTGTTGCAGTTCACGGCAGATACACTTGCTGTTGCGGTGCTGATTACGACCGCTCACTCGTGGCAGCA |
| | ACAGGGGAAAACCTTATTTATCAGCCGGAAAACCTACCGGATTGTTGGTAGTGGTCAATAGGCGATTACCGTTGTGTTGAAGTGGCGAGCGATACACCGCTTCCGGCGCGGATTGGCCTGAACTGCCAACTGGCGCAGGT |
| | AGCAGAGCGGGTAAACTGGCTCGGATTAGGGCCGCAAGAAAACTATCCCGACCGCCTTACTGCCGCCTGTTTTGACCGCTGGGATCTGCCAAGTCAGACAGTATAGCCCGTACGTCTTCCCGAGCGAAAACGGTCTGCGC |
| | TGCGGGACGCGCGAATTGAATTTGGCCCACACCAGTGGCGCGGCGACTTCCAGTTCAATATCAGCCGCTACAGTGAACAGCAACTGTTGGAAACCAGCCTTCGCCAACTGCTGCACGCGGAAGAAGGCACTGGCTGAATA |
| | TCGACGGTTTCCAGTTGGGGATTGGTGGCGACGACTCCTGGAGCCCGTCAGTATCGGCGGACTTCCAACTGAGCGCCGGTCGCTACCTTACCAGTTGGTCTGGTGTCAAAAAGCGTCCGCTTGAGTCTAGCGATCGCGCG |
| | CAGATCTGTCATGTGAGCAAAAGGCCAGCAAAAGGCCAGGAACCGTAAAAAGGCCGCGTTGCTGGCGTTTTTCCATAGGCTCCGCCCCCCTGACGAGCATCACAAAAATCGACGCTCAAGTCAGAGGTGGCGAAACCCGA |
| | CAGGACTATAAAGATACCAGGCGTTTCCCCCTGGAAGCTCCCTCGTGCGCTCTCCTGTTCCGACCCTGCCGCTTACCGGATACCTGTCCGCCTTTCTCCCTTCGGGAAGCGTGGCGCTTTCTCATAGCTCACGCTGTAGG |
| | TATCTCAGTTCGGTGTAGGTCGTTCGCTCCAAGCTGGGCTGTGTGCACGAACCCCCCGTTCAGCCCGACCGCTGCGCCTTATCCGGTAACTATCGTCTTGAGTCCAACCCGGTAAGACACGACTTATCGCCACTGGCAGC |
| | AGCCACTGGTAACAGGATTAGCAGAGCGAGGTATGTAGGCGGTGCTACAGAGTTCTTGAAGTGGTGGCCTAACTACGGCTACACTAGAAGAACAGTATTTGGTATCTGCGCTCTGCTGAAGCCAGTTACCTTCGGAAAAA |
| | GAGTTGGTAGCTCTTGATCCGGCAAACAAACCACCGCTGGTAGCGGTGGTTTTTTTGTTTGCAAGCAGCAGATTACGCGCAGAAAAAAAGGATCTCAAGAAGATCCTTTGATCTTTTCTACGGGGTCTGACGCTCAGTGG |
| | AACGAAAACTCACGTTAAGGGATTTTGGTCATGAGATTATCAAAAAGGATCTTCACCTAGATCCTTTTCACGTAGAAAGCCAGTCCGCAGAAACGGTGCTGACCCCGGATGAATGTCAGCTACTGGGCTATCTGGACAAG |
| | GGAAAACGCAAGCGCAAAGAGAAAGCAGGTAGCTTGCAGTGGGCTTACATGGCGATAGCTAGACTGGGCGGTTTTATGGACAGCAAGCGAACCGGAATTGCCAGCTGGGGCGCCCTCTGGTAAGGTTGGGAAGCCCTGCA |
| | AAGTAAACTGGATGGCTTTCTTGCCGCCAAGGATCTGATGGCGCAGGGGATCAAGATCTGATCAAGAGACAGGATGAGGATCGTTTCGCATGATTGAACAAGATGGATTGCACGCAGGTTCTCCGGCCGCTTGGGTGGAG |
| | AGGCTATTCGGCTATGACTGGGCACAACAGACAATCGGCTGCTCTGATGCCGCCGTGTTCCGGCTGTCAGCGCAGGGGCGCCCGGTTCTTTTTGTCAAGACCGACCTGTCCGGTGCCCTGAATGAACTGCAAGACGAGGC |
| | AGCGCGGCTATCGTGGCTGGCCACGACGGGCGTTCCTTGCGCAGCTGTGCTCGACGTTGTCACTGAAGCGGGAAGGGACTGGCTGCTATTGGGCGAAGTGCCGGGGCAGGATCTCCTGTCATCTCACCTTGCTCCTGCCG |
| | AGAAAGTATCCATCATGGCTGATGCAATGCGGCGGCTGCATACGCTTGATCCGGCTACCTGCCCATTCGACCACCAAGCGAAACATCGCATCGAGCGAGCACGTACTCGGATGGAAGCCGGTCTTGTCGATCAGGATGAT |
| | CTGGACGAAGAGCATCAGGGGCTCGCGCCAGCCGAACTGTTCGCCAGGCTCAAGGCGAGCATGCCCGACGGCGAGGATCTCGTCGTGACCCATGGCGATGCCTGCTTGCCGAATATCATGGTGGAAAATGGCCGCTTTTC |
| | TGGATTCATCGACTGTGGCCGGCTGGGTGTGGCGGACCGCTATCAGGACATAGCGTTGGCTACCCGTGATATTGCTGAAGAGCTTGGCGGCGAATGGGCTGACCGCTTCCTCGTGCTTTACGGTATCGCCGCTCCCGATT |
| | CGCAGCGCATCGCCTTCTATCGCCTTCTTGACGAGTTCTTCTGAATTTAAAGCCCAATACGCAAACCGCCTCTCCCCGCGCGTTGGCCGAAGCGGTTCAATATTTTGTTAAAATTCGCGTTAAATTTTTGTTAAATCAGC |
| | TATTTTTTAACCAATAGGCCGAAATCGGCAAAATCCCTTGTAAATCAAAAGAATAGACCGAGATAGGGTTGAGTGTTGTTCCAGTTTGGAACAAGAGTCCACTATTAAAGAACGTGGACTCCAACGTCAAAGGGCGAAAA |
| | ACCGTCTATCAGGGCGTTGGCCCACTACGTGAACCTTCACCCTAATCAAGTTTTTTGGGGTCGAGGTGCCGTAAAGCACTAAATCGGAACCCTAAAGGGAGCCCCCGATTTAGAGCTTGACGGGGAAACCGGCGAACGTG |
| | GCGAGAAAGGAAGGGAAGAAAGCGAAAGGAGCGGGCGCTAGGGCGCTGGCAAGTGTAGCGGTCACGCTGCGCGTAACCACCACACCCGCCGCGCTAAGCGCCGCTACAGGGCGCGTCCCTTCGCCTTCAGGCTGCGTCGA |
| | GTACTGTACTGTGAGCCAGAGTTGCCCGGCGCTCTCCGGCTGCGGTAGTTCAGGCAGTTCAATCAACTGTTTACCTTGTGGAGCGACTCCAGAGGCACTTCACCGCTTGCCAGCGGCTTACGATCCAGCGCCACGATCCA |
| | GTGCAGGAGATCGTTATCGCTATACGGAACAGGTATTCGCTGGTCACTTCGATAAGGTTTGCCCGGATAAACGGAACTGGAAAAACTGCTGCTGGTGTTTTGCTTCCGTCAGTGCTGGATCGGCGTGCGGTCGGCAAAGA |
| | CCAGACCGTTCTAACAGAACTGGCGATTGTTCGGCGTATCGCCAAAATCACCGCCGTAAGCCGACCACGGGTTGCCGTTTTCAGCAGGATTTAATCAGCGACTGATCCACCCAGTCCCAGACGAAGCCGCCCTGTAAACG |
| | GGGATACTGACGAAACGCCTGCCAGTATTTAGCGAAACCGCCAAGACTGTTACCCAAGCGTGGGCGTATTCGCAAAGGATCAGCGGGCGCGTCTCTCCAGGTAGCGAAAGCCTTTTTTGATCGACCTTTCGGCACAGCCG |
| | GGAAGGGCTGGTCTTCAACCACGCGCGCGTACAACGGGCAAATAATATCGGTGGCCGTGGTGTCGGCTCCGCCGCCTTCAACTGCACCGGGCGGGAAGGATCGACAGATTTGATCCAGCGATACAGCGCGTCGTGATTAG |
| | CGCCGTGGCCTGATTCAATTCCCCAGCGACCAGTAGATCACACTCGGGTGATTACGATTGCGCTGCACCAGTCGCGTTACGGTTCGCTCTTCGCCGGTAGCCAGCGCGGATCACGGTCAGACGATTCGTTGGCACGATCC |
| | GTGGGTTTCAATACTGGCTTCAAACCACCACTAACAGGCCGTAGCGGTCGCACAGCGTGTACCACAGCGGTTGGTTCGGATAATCGAACAGCGCACGGCGTTAAAGTTGTTCTGCTTCAACAGCAGGATATTCTGCACCT |
| | TCGTCTGCTCTTCCTAACCTGACCAAGCAGAGGATCTGCTCGTGACGGTTAATCCTCGAATCAGCAACGGCTTGCCGTTCAGCAGCAGCAGACCAAGTTCAATCCGCACCTCGCGGAAACCGACAACGCAGGCTTCTGCT |
| | TCAATCAGCGTGCCGTCGGCGGTGTGCAGTTCAACCACCGCACGATAGAGATTCGGGATTTCGGCGCTCCACAGTTTCGGGTTTTCGACGTTCAGACGTAGTGTGACGCGATCTGCAAACCACCACGCTCAACGATAATT |
| | TCACCGCCGAAAGGCGCGGTGCCGCTGGCGACCTGCGTTTCACCCTGCCAGAAAGAAACTGTTACCCGTAGGTAGTCACGCAACTCGCCGCACACTGAACTTCAGCCTCCAGTACAGCGCGGCTGAAATCGTCTTAAAGC |
| | GAGTGGCAACTGGAAATCGCTGATTTGTGTAGTCGGTTTAGCAGCAACGAGACTTCACGGAAAATCCGCTAATCCGCCACAGATCCTGATCTTCCAGATAACTGCCGTCACTCCAACGCAGCACCTTCACCGCGAGGCGG |
| | TTTTCTCCGGCGCGTAAAAATCGCTCAGGTCAAATTCAGACGGCAAACGACTGTCCTGGCCGTAACCGACCCAGCGCCCGTTGCACCACAGATTGAAACGCCGAGTTTACGCCTCAAAAATAATTCGCGTCTGGCCTTCC |
| | TGTAGCCAGCTTTCACAACTATAATAGTGAGCGAGTAACAACCCGTCGGATTCTCCGTGGGAACAAACGGCGGATTGACCGTATAGGGATAGGTTACGTTGGTGTAGTAGGGCGCTCCGTAACCGTGCTACTGCCAGTTT |
| | GAGGGGACGACGACAGTATCGGCCTCAGGAAGATCGCACTCCAGCCAGCTTTCCGGCACCGCTTCTGGTACTGGAAACCAGGCAAAGCGCCTATCGCCTATCAGGCTGCACAACTGTTGGGAAGGGCGATCTGTGCGGGC |
| | CTCTTCGCTATTACGCCAGCTTGCGAAAGGGGGTAGTGCTGCAAGGCGATTAAGTTGGGTAACGCCAGGGTTTTCCCAGTCACGACGTGGATCTGGGCCACTCCCTCT |

| pP065_seq100_29-56 deletion (SEQ ID NO: 470) | |
|---|---|
| (SEQ ID NO: 470) | |
| 1 | tctagagcta gcatatggat ccatcgattt agggataaca gggtaattat cagcacacaa |
| 61 | ttgcccatta tacgcgcgta taatggacta ttgtgtgctg atatctgtac acttaagggc |
| 121 | tagatcttag cttacgtcac tagagggtcc acgtttagtt tttaagatcc attgatctcc |
| 181 | taaacgctgc aagattcgca acctggtata cttagcctag gcgctaggtc ctagtgcagc |
| 241 | gggacttttt ttctaaagtc gttgagagga ggagtcgtca gaccagatag ctttgatgtc |
| 301 | ctgatcggaa ggatcgttgg cccccctgca ggcagctgtt aattaactgc gcgctcgctc |
| 361 | gctcactgag gccgcccggg caaagcccgg gcgtcgggcg acctttggtc gcccggcctc |
| 421 | agtgagcgag cgagcgcgca gagagggagt ggccaactcc atcactaggg gttccttgta |
| 481 | gttaatgatt aacccgccat gctacttatc tacggcgcgc cacgcgttct aggagttaat |
| 541 | ttttaaaaag cagtcaaaag tccaagtgcc cttgcgagca tttactctct ctgtttgctc |
| 601 | tggttaataa tctcaggagc acaaacattc cttactagtt ctaggagtta atttttaaaa |
| 661 | agcagtcaaa agtccaagtg cccttgcgag catttactct ctctgtttgc tctggttaat |
| 721 | aatctcagga gcacaaacat tccttactag ttctagagcg gccgccagtg tgctggaatt |
| 781 | cggctttttt agggctggaa gctacctttg acatcatttc ctctgcgaat gcatgtataa |
| 841 | tttctacaga acctattaga aaggatcacc cagcctctgc ttttgtacaa ctttccctta |
| 901 | aaaaactgcc aatcccactg ctgtttggcc caatagtgag aactttttcc tgctgcctct |
| 961 | tggtgctttt gcctatggcc cctattctgc ctgctgaaga cactcttgcc agcatggact |
| 1021 | taaacccctc cagctctgac aatcctcttt ctcttttgtt ttacatgaag ggtctggcag |
| 1081 | ccaaagcaat cactcaaagt tcaaacctta tcattttttg ctttgttcct cttggccttg |
| 1141 | gttttgtaca tcagctttga aaataccatc ccagggttaa tgctggggtt aatttataac |
| 1201 | tgagagtgct ctagttctgc aatacaggac atgctataaa aatggaaaga tgttgctttc |
| 1261 | tgagagatca ggtggattct tgggcatttg cttaagcttg gtaccgccac catgggggtg |
| 1321 | aggcaccctc cctgttctca caggctgctg gctgtgtgtg ctctggtgtc tctggccact |
| 1381 | gctgccctgc tgggccagca gggggccagc aggccaggcc ccagggatgc ccaggcccac |
| 1441 | cctggcagac ccagggctgt gcccacccag tgtgatgtgc ctcccaacag cagatttgac |
| 1501 | tgtgcccctg ataaggccat tactcaggaa cagtgtgagg ccaggggctg ctgctatatc |
| 1561 | cctgccaagc agggcctgca gggggcccag atggggcagc cctggtgctt cttccccccc |
| 1621 | agctacccct cttataagct ggagaatctg agcagctctg aaatgggcta cactgccact |
| 1681 | ctgaccagga ctacccccac cttcttcccc aaggatattc tgactctgag gctggatgtg |
| 1741 | atgatggaga ctgagaacag gctgcacttc accatcaagg accctgccaa caggaggtat |
| 1801 | gaggtgcccc tggaaactcc cagggtgcac tctagggccc ccagccccct gtattctgtg |
| 1861 | gagttctctg aggagccttt tggggtcatt gtccacaggc agctggatgg cagggtgctg |
| 1921 | ctgaatacta ctgtggcccc tctgttcttt gctgaccagt tcctgcagct gagcaccagc |
| 1981 | ctgccttctc agtacattac tggcctggct gagcatctga gccccctgat gctgagcacc |
| 2041 | tcttggacca gaatcaccct gtggaacagg gacctggctc ccactcctgg ggccaacctg |
| 2101 | tatggcagcc accccttcta cctggccctg gaggatgggg gctctgccca tggggtgttc |
| 2161 | ctgctgaaca gcaatgccat ggatgtggtg ctgcagccct ctcctgccct gtcttggaga |
| 2221 | tctactgggg gcatcctgga tgtgtatatc ttcctggggc ctgagcccaa gtctgtggtg |
| 2281 | cagcagtacc tggatgtggt gggctacccc ttcatgcccc cctactgggg cctgggcttc |
| 2341 | cacctgtgca ggtggggcta ctcttctact gctatcacca ggcaggtggt ggagaacatg |
| 2401 | accagggctc acttccctct ggatgtgcag tggaatgacc tggactacat ggactctagg |
| 2461 | agagacttca cttttaataa ggatgggttc agggactttc ctgccatggt gcaggagctg |
| 2521 | catcaggggg gcaggagata tatgatgatt gtggaccctg ctatttctag ctctggccct |
| 2581 | gctggcagct ataggcccta tgatgagggg ctgaggaggg gggtgttcat cactaatgag |
| 2641 | actggccagc ccctgattgg caaggtgtgg cctggctcta ctgccttccc tgatttcacc |
| 2701 | aaccccactg ccctggcctg gtgggaggat atggtggctg agtttcatga ccaggtgccc |
| 2761 | tttgatggca tgtggattga catgaatgag cccagcaact ttatcagggg ctctgaagat |
| 2821 | ggctgcccca acaatgagct ggagaacccc ccctatgtgc ctggggtggt ggggggcacc |
| 2881 | ctgcaggctg ccaccatctg tgccagcagc caccagttcc tgagcaccca ctataacctg |
| 2941 | cacaacctgt atggcctgac tgaggccatt gcctctcaca gggccctggt gaaggctagg |
| 3001 | gggactaggc cctttgtgat cagcaggtct acttttgctg gccatggcag gtatgctggg |
| 3061 | cactggactg gggatgtgtg gtctagctgg gagcagctgg ccagctctgt gcctgagatc |
| 3121 | ctgcagttta atctgctggg ggtgcccctg gtgggggctg atgtgtgtgg cttcctgggc |
| 3181 | aatacctctg aggagctgtg tgtgaggtgg actcagctgg gggctttcta ccccttcatg |
| 3241 | agaaaccaca actctctgct gagcctgccc caggagccct attctttttc tgagcctgcc |
| 3301 | cagcaggcta tgaggaaggc cctgactctg aggtatgccc tgctgcccca cctgtatacc |
| 3361 | ctgttccatc aggcccatgt ggctggggag actgtggcca gacctctgtt cctggagttc |
| 3421 | cccaaggata gctctacttg gactgtggac caccagctgc tgtgggggga ggctctgctg |
| 3481 | atcacccctg tgctgcaggc tgggaaggct gaggtgactg gctatttccc cctgggcacc |
| 3541 | tggtatgatc tgcagactgt gcccattgag gccctgggct ctctgccccc tccccctgct |
| 3601 | gcccccaggg agcctgccat ccactctgag ggccagtggg tgaccctgcc tgcccctctg |
| 3661 | gacactatca atgtgcacct gagggctggc tacatcatcc ccctgcaggg ccctggcctg |
| 3721 | actaccactg agtctaggca gcagcccatg gccctggctg tggctctgac caaggggggg |
| 3781 | gaggccaggg gggagctgtt ctgggatgat ggggagtctc tggaggtgct ggagaggggg |
| 3841 | gcctacaccc aggtgatctt cctggctagg aataacacca ttgtcaatga gctggtgagg |
| 3901 | gtgacctctg agggggctgg cctgcagctg cagaaggtga ctgtgctggg ggtggctact |
| 3961 | gccccccagc aggtgctgag caatggggtg cctgtgagca acttcaccta tagccctgac |
| 4021 | accaaggtgc tggacatctg tgtgagcctg ctgatggggg agcagttcct ggtgagctgg |
| 4081 | tgctaattta aatctcgagc cgggcggagt gtgttagtct ctccagaggg aggctggttc |
| 4141 | cccagggaag cagagcctgt gtgcgggcag cagctgtgtg cgggcctggg ggttgttaag |
| 4201 | tgcaattatt tttaataaaa ggggcatttg gaaaaaaaaa aaaaaggtag cagtcgacag |
| 4261 | atgaattctg cagatctgtg gcttctagct gcccgggtgg catccctgtg acccctcccc |
| 4321 | agtgcctctc ctggccctgg aagttgccac tccagtgccc accagccttg tcctaataaa |
| 4381 | attaagttgc atcattttgt ctgactaggt gtccttctat aatattatgg ggtggagggg |
| 4441 | ggtggtatgg agcaaggggc aagttgggaa gacaacctgt agggcctgcg gggtctattg |
| 4501 | ggaaccaagc tggagtgcag tggcacaatc ttggctcact gcaatctccg cctcctgggt |
| 4561 | tcaagcgatt ctcctgcctc agcctcccga gttgttggga ttccaggcat gcatgaccag |
| 4621 | gctcagctaa tttttgtttt tttggtagag acggggtttc accatattgg ccaggctggt |
| 4681 | ctccaactcc taatctcagg tgatctaccc accttggcct cccaaattgc tgggattaca |
| 4741 | ggcgtgaacc actgctccct tccctgtcct tctgatttta aaaaaaagag aaatgttctg |
| 4801 | gcacctgcac ttgcactggg gacagcctat tttgctagtt tgttttgttt cgttttgttt |
| 4861 | tgatggagag cgtatgttgt ttaaacgcgg ccgcgtagat aagtagcatg gcgggttaat |
| 4921 | cattaactac aaggaacccc tagtgatgga gttggccact ccctctctgc gcgctcgctc |
| 4981 | gctcactgag gccgggcgac caaaggtcgc ccgacgcccg ggctttgccc gggcggcctc |
| 5041 | agtgagcgag cgagcgcgca gttaattaag gcgccctagg ccgaccctta gactctgtac |
| 5101 | tcagttctat aaacgagcca ttggatacga gatccgtaga ttgataaggg acacggaata |
| 5161 | tccccggacg caatagacac cggtggacag cttggtatcc tgagcacagt cgcgcgtccg |
| 5221 | aatctagctc tactttagag gccccggatt ctgatggtcg tagaccgcag aaccgattgg |
| 5281 | ggggatgaga tctactagtt atcagcacac aattgcccat tatacgcgcg tataatggac |
| 5341 | tattgtgtgc tgatataggg ataacagggt aattctagag ctagcatatg gatccatcga |
| 5401 | tttgatgcgg tattttctcc ttacgcatct gtgcggtatt tcacaccgca tacgtcaaag |
| 5461 | caaccatagt acgcgccctg tagcggcgca ttaagcgcgg cgggtgtggt ggttacgcgc |
| 5521 | agcgtgaccg ctacacttgc cagcgcccta gcgcccgctc ctttcgcttt cttcccttcc |
| 5581 | tttctcgcca cgttcgccgg ctttccccgt caagctctaa atcgggggct ccctttaggg |
| 5641 | ttccgattta gtgctttacg gcacctcgac cccaaaaaac ttgatttggg tgatggttca |
| 5701 | cgtagtgggc catcgccctg atagacggtt tttcgccctt tgacgttgga gtccacgttc |
| 5761 | tttaatagtg gactcttgtt ccaaactgga acaacactca actctatctc gggctattct |
| 5821 | tttgatttat aagggatttt gccgatttcg gtctattggt taaaaaatga gctgatttaa |
| 5881 | caaaaattta acgcgaattt taacaaaata ttaacgttta caattttatg gtgcactctc |
| 5941 | agtacaatct gctctgatgc cgcatagtta agccagcccc gacacccgcc aacacccgct |
| 6001 | gacgcgccct gacgggcttg tctgctcccg gcatccgctt acagacaagc tgtgaccgtc |
| 6061 | tccgggagct gcatgtgtca gaggttttca ccgtcatcac cgaaacgcgc gagacgaaag |
| 6121 | ggcctcgtga tacgcctatt tttataggtt aatgtcatga taataatggt ttcttagacg |
| 6181 | tcaggtggca cttttcgggg aaatgtgcgc ggaaccccta tttgtttatt tttctaaata |
| 6241 | cattcaaata tgtatccgct catgagacaa taaccctgat aaatgcttca ataatattga |
| 6301 | aaaaggaaga gtatgagcca tattcaacgg gaaacgtcga ggccgcgatt aaattccaac |
| 6361 | atggatgctg atttatatgg gtataaatgg gctcgcgata atgtcgggca atcaggtgcg |
| 6421 | acaatctatc gcttgtatgg gaagcccgat gcgccagagt tgtttctgaa acatggcaaa |
| 6481 | ggtagcgttg ccaatgatgt tacagatgag atggtcagac taaactggct gacggaattt |
| 6541 | atgcctcttc cgaccatcaa gcattttatc cgtactcctg atgatgcatg gttactcacc |
| 6601 | actgcgatcc ccggaaaaac agcattccag gtattagaag aatatcctga ttcaggtgaa |
| 6661 | aatattgttg atgcgctggc agtgttcctg cgccggttgc attcgattcc tgtttgtaat |
| 6721 | tgtcctttta acagcgatcg cgtatttcgt ctcgctcagg cgcaatcacg aatgaataac |
| 6781 | ggtttggttg atgcgagtga ttttgatgac gagcgtaatg gctggcctgt tgaacaagtc |
| 6841 | tggaaagaaa tgcataaact tttgccattc tcaccggatt cagtcgtcac tcatggtgat |
| 6901 | ttctcacttg ataaccttat ttttgacgag gggaaattaa taggttgtat tgatgttgga |
| 6961 | cgagtcggaa tcgcagaccg ataccaggat cttgccatcc tatggaactg cctcggtgag |
| 7021 | ttttctcctt cattacagaa acggcttttt caaaaatatg gtattgataa tcctgatatg |
| 7081 | aataaattgc agtttcattt gatgctcgat gagtttttct aagcgtataa tggtctagag |
| 7141 | ctagcatatg gatccatcga ttccattata cgcctgtcag accaagttta ctcatatata |
| 7201 | ctttagattg atttaaaact tcatttttaa tttaaaagga tctaggtgaa gatccttttt |
| 7261 | gataatctca tgaccaaaat cccttaacgt gagttttcgt tccactgagc gtcagacccc |
| 7321 | gtagaaaaga tcaaaggatc ttcttgagat cctttttttc tgcgcgtaat ctgctgcttg |
| 7381 | caaacaaaaa aaccaccgct accagcggtg gtttgtttgc cggatcaaga gctaccaact |
| 7441 | ctttttccga aggtaactgg cttcagcaga gcgcagatac caaatactgt tcttctagtg |
| 7501 | tagccgtagt taggccacca cttcaagaac tctgtagcac cgcctacata cctcgctctg |
| 7561 | ctaatcctgt taccagtggc tgctgccagt ggcgataagt cgtgtcttac cgggttggac |
| 7621 | tcaagacgat agttaccgga taaggcgcag cggtcgggct gaacgggggg ttcgtgcaca |
| 7681 | cagcccagct tggagcgaac gacctacacc gaactgagat acctacagcg tgagctatga |
| 7741 | gaaagcgcca cgcttcccga agggagaaag gcggacaggt atccggtaag cggcagggtc |
| 7801 | ggaacaggag agcgcacgag ggagcttcca gggggaaacg cctggtatct ttatagtcct |
| 7861 | gtcgggtttc gccacctctg acttgagcgt cgatttttgt gatgctcgtc aggggggcgg |
| 7921 | agcctatgga aaaacgccag caacgcggcc tttttacggt tcctggcctt ttgctggcct |
| 7981 | tttgctcaca |

| pP066_seq3_29-56 deletion (SEQ ID NO: 471) | |
|---|---|
| (SEQ ID NO: 471) | |
| 1 | tctagagcta gcatatggat ccatcgattt agggataaca gggtaattat cagcacacaa |
| 61 | ttgcccatta tacgcgcgta taatggacta ttgtgtgctg atatctgtac acttaagggc |
| 121 | tagatcttag cttacgtcac tagagggtcc acgtttagtt tttaagatcc attgatctcc |
| 181 | taaacgctgc aagattcgca acctggtata cttagcctag gcgctaggtc ctagtgcagc |
| 241 | gggacttttt ttctaaagtc gttgagagga ggagtcgtca gaccagatag ctttgatgtc |
| 301 | ctgatcggaa ggatcgttgg cccccctgca ggcagctgtt aattaactgc gcgctcgctc |
| 361 | gctcactgag gccgcccggg caaagcccgg gcgtcgggcg acctttggtc gcccggcctc |
| 421 | agtgagcgag cgagcgcgca gagagggagt ggccaactcc atcactaggg gttccttgta |
| 481 | gttaatgatt aacccgccat gctacttatc tacggcgcgc cacgcgttct aggagttaat |
| 541 | ttttaaaaag cagtcaaaag tccaagtgcc cttgcgagca tttactctct ctgtttgctc |
| 601 | tggttaataa tctcaggagc acaaacattc cttactagtt ctaggagtta atttttaaaa |
| 661 | agcagtcaaa agtccaagtg cccttgcgag catttactct ctctgtttgc tctggttaat |
| 721 | aatctcagga gcacaaacat tccttactag ttctagagcg gccgccagtg tgctggaatt |
| 781 | cggctttttt agggctggaa gctacctttg acatcatttc ctctgcgaat gcatgtataa |
| 841 | tttctacaga acctattaga aaggatcacc cagcctctgc ttttgtacaa ctttccctta |
| 901 | aaaaactgcc aatcccactg ctgtttggcc caatagtgag aactttttcc tgctgcctct |
| 961 | tggtgctttt gcctatggcc cctattctgc ctgctgaaga cactcttgcc agcatggact |
| 1021 | taaacccctc cagctctgac aatcctcttt ctcttttgtt ttacatgaag ggtctggcag |
| 1081 | ccaaagcaat cactcaaagt tcaaacctta tcattttttg ctttgttcct cttggccttg |
| 1141 | gttttgtaca tcagctttga aaataccatc ccagggttaa tgctggggtt aatttataac |
| 1201 | tgagagtgct ctagttctgc aatacaggac atgctataaa aatggaaaga tgttgctttc |
| 1261 | tgagagatca ggtggattct tgggcatttg cttaagcttg gtaccgccac catgggagtg |
| 1321 | agacatcctc cttgttctca tagactgctg gctgtgtgtg ctctggtgtc tctggctaca |
| 1381 | gctgctctgc tgggacagca gggagcttct agacctggac ctagagatgc tcaggctcat |
| 1441 | cctggaagac ctagagctgt gcctacacag tgtgatgtgc ctcctaattc tagatttgat |
| 1501 | tgtgctcctg ataaggctat aacacaggag cagtgtgagg ctagaggatg ttgttatatt |
| 1561 | cctgctaagc agggactgca gggagctcag atgggacagc cttggtgttt ttttcctcct |
| 1621 | tcttatcctt cttataagct ggagaatctg tcttcttctg agatgggata tacagctaca |
| 1681 | ctgacaagaa caacacctac attttttcct aaggatattc tgacactgag actggatgtg |
| 1741 | atgatggaga cagagaatag actgcatttt acaattaagg atcctgctaa tagaagatat |
| 1801 | gaggtgcctc tagagacacc tagagtgcat tctagagctc cttctcctct gtattctgtg |
| 1861 | gagttttctg aggagccttt tggagtgatt gtgcatagac agctggatgg aagagtgctg |
| 1921 | ctgaatacaa cagtggctcc tctgtttttt gctgatcagt ttctgcagct gtctacatct |
| 1981 | ctgccttctc agtatattac aggactggct gagcatctgt ctcctctgat gctgtctaca |
| 2041 | tcttggacaa gaattacact gtggaataga gatctggctc ctacacctgg agctaatctg |
| 2101 | tatggatctc atccttttta tctggctctg gaggatggag gatctgctca tggagtgttt |
| 2161 | ctgctgaatt ctaatgctat ggatgtagtg ctgcagcctt ctcctgctct gtcttggaga |
| 2221 | tctacaggag gaattctgga tgtgtatatt tttctaggac ctgagcctaa gtctgtggtg |
| 2281 | cagcagtatc tggatgtagt gggatatcct tttatgcctc cttattgggg actgggattt |
| 2341 | catctgtgta gatggggata ttcttctaca gctattacaa gacaggtggt tgagaatatg |
| 2401 | acaagagctc attttcctct ggatgtgcag tggaatgatc tagattatat ggattctaga |
| 2461 | agagatttta catttaataa ggatggattt agagattttc ctgctatggt gcaggagctg |
| 2521 | catcagggag gaagaagata tatgatgatt gtggatcctg ctatttcttc ttctggacct |
| 2581 | gctggatctt atagacctta tgatgaggga ctgagaagag gagtgtttat tacaaatgag |
| 2641 | acaggacagc ctctgattgg aaaggtgtgg cctggatcta cagcttttcc tgattttaca |
| 2701 | aatcctacag ctctggcttg gtgggaggat atggtggctg agtttcatga tcaggtgcct |
| 2761 | tttgatggaa tgtggattga tatgaatgag ccttctaatt ttataagagg atctgaggat |
| 2821 | ggatgtccta ataatgagct agagaatcct ccttatgtgc ctggagtagt gggaggaaca |
| 2881 | ctgcaggctg ctacaatttg tgcttcttct catcagtttc tgtctacaca ttataatctg |
| 2941 | cataatctgt atggactgac agaggctatt gcttctcata gagctctggt gaaggctaga |
| 3001 | ggaacaagac cttttgtgat ttctagatct acatttgctg gacatggaag atatgctgga |
| 3061 | cattggacag gagatgtatg gtcttcttgg gagcagctag cttcttctgt gcctgagatt |
| 3121 | ctgcagttta atctgctagg agtgcctctg gtgggagctg atgtgtgtgg atttctggga |
| 3181 | aatacatctg aggagctgtg tgtgagatgg acacagctgg gagcttttta tccttttatg |
| 3241 | agaaatcata attctctgct gtctctgcct caggagcctt attctttttc tgagcctgct |
| 3301 | cagcaggcta tgagaaaggc tctgacactg agatatgctc tgctgcctca tctgtataca |
| 3361 | ctgtttcatc aggctcatgt ggctggagag acagtagcta gacctctgtt tctggagttt |
| 3421 | cctaaggatt cttctacatg gacagttgat catcagctgc tgtggggaga ggctctgctg |
| 3481 | attacacctg tgctgcaggc tggaaaggct gaggtgacag gatattttcc tctgggaaca |
| 3541 | tggtatgatc tgcagacagt gcctatagag gctctgggat ctctgcctcc tcctcctgct |
| 3601 | gctcctagag agcctgctat tcattctgag ggacagtggg tgacactgcc tgctcctctg |
| 3661 | gatacaatta atgtgcatct gagagctgga tatattattc ctctgcaggg acctggactg |
| 3721 | acaacaacag agtctagaca gcagcctatg gctctggctg tggctctgac aaagggagga |
| 3781 | gaggctagag gagagctgtt ttgggatgat ggagagtctc tggaggtgct ggagagagga |
| 3841 | gcttatacac aggtgatttt tctggctaga aataatacaa ttgtgaatga gctggtgaga |
| 3901 | gtgacatctg agggagctgg actgcagctg cagaaggtga cagtgctggg agtggctaca |
| 3961 | gctcctcagc aggtgctgtc taatggagtg cctgtgtcta attttacata ttctcctgat |
| 4021 | acaaaggtgc tggatatttg tgtgtctctg ctgatgggag agcagtttct ggtgtcttgg |
| 4081 | tgttaatgat ttaaatctcg agccgggcgg agtgtgttag tctctccaga gggaggctgg |
| 4141 | ttccccaggg aagcagagcc tgtgtgcggg cagcagctgt gtgcgggcct gggggttgtt |
| 4201 | aagtgcaatt atttttaata aaaggggcat ttggaaaaaa aaaaaaaagg tagcagtcga |
| 4261 | cagatgaatt ctgcagatct gtggcttcta gctgcccggg tggcatccct gtgacccctc |
| 4321 | cccagtgcct ctcctggccc tggaagttgc cactccagtg cccaccagcc ttgtcctaat |
| 4381 | aaaattaagt tgcatcattt tgtctgacta ggtgtccttc tataatatta tggggtggag |
| 4441 | gggggtggta tggagcaagg ggcaagttgg gaagacaacc tgtagggcct gcggggtcta |
| 4501 | ttgggaacca agctggagtg cagtggcaca atcttggctc actgcaatct ccgcctcctg |
| 4561 | ggttcaagcg attctcctgc ctcagcctcc cgagttgttg ggattccagg catgcatgac |
| 4621 | caggctcagc taatttttgt ttttttggta gagacggggt ttcaccatat tggccaggct |
| 4681 | ggtctccaac tcctaatctc aggtgatcta cccaccttgg cctcccaaat tgctgggatt |
| 4741 | acaggcgtga accactgctc ccttccctgt ccttctgatt ttaaaaaaaa gagaaatgtt |
| 4801 | ctggcacctg cacttgcact ggggacagcc tattttgcta gtttgttttg tttcgttttg |
| 4861 | ttttgatgga gagcgtatgt tgtttaaacg cggccgcgta gataagtagc atggcgggtt |
| 4921 | aatcattaac tacaaggaac ccctagtgat ggagttggcc actccctctc tgcgcgctcg |
| 4981 | ctcgctcact gaggccgggc gaccaaaggt cgcccgacgc ccgggctttg cccgggcggc |
| 5041 | ctcagtgagc gagcgagcgc gcagttaatt aaggcgccct aggccgaccc ttagactctg |
| 5101 | tactcagttc tataaacgag ccattggata cgagatccgt agattgataa gggacacgga |
| 5161 | atatccccgg acgcaataga caccggtgga cagcttggta tcctgagcac agtcgcgcgt |
| 5221 | ccgaatctag ctctacttta gaggccccgg attctgatgg tcgtagaccg cagaaccgat |
| 5281 | tggggggatg agatctacta gttatcagca cacaattgcc cattatacgc gcgtataatg |
| 5341 | gactattgtg tgctgatata gggataacag ggtaattcta gagctagcat atggatccat |
| 5401 | cgatttgatg cggtattttc tccttacgca tctgtgcggt atttcacacc gcatacgtca |
| 5461 | aagcaaccat agtacgcgcc ctgtagcggc gcattaagcg cggcgggtgt ggtggttacg |
| 5521 | cgcagcgtga ccgctacact tgccagcgcc ctagcgcccg ctcctttcgc tttcttccct |
| 5581 | tcctttctcg ccacgttcgc cggctttccc cgtcaagctc taaatcgggg gctcccttta |
| 5641 | gggttccgat ttagtgcttt acggcacctc gaccccaaaa aacttgattt gggtgatggt |
| 5701 | tcacgtagtg ggccatcgcc ctgatagacg gtttttcgcc ctttgacgtt ggagtccacg |
| 5761 | ttctttaata gtggactctt gttccaaact ggaacaacac tcaactctat ctcgggctat |
| 5821 | tcttttgatt tataagggat tttgccgatt tcggtctatt ggttaaaaaa tgagctgatt |
| 5881 | taacaaaaat ttaacgcgaa ttttaacaaa atattaacgt ttacaatttt atggtgcact |
| 5941 | ctcagtacaa tctgctctga tgccgcatag ttaagccagc cccgacaccc gccaacaccc |
| 6001 | gctgacgcgc cctgacgggc ttgtctgctc ccggcatccg cttacagaca agctgtgacc |
| 6061 | gtctccggga gctgcatgtg tcagaggttt tcaccgtcat caccgaaacg cgcgagacga |
| 6121 | aagggcctcg tgatacgcct atttttatag gttaatgtca tgataataat ggtttcttag |
| 6181 | acgtcaggtg gcacttttcg gggaaatgtg cgcggaaccc ctatttgttt atttttctaa |
| 6241 | atacattcaa atatgtatcc gctcatgaga caataaccct gataaatgct tcaataatat |
| 6301 | tgaaaaagga agagtatgag ccatattcaa cgggaaacgt cgaggccgcg attaaattcc |
| 6361 | aacatggatg ctgatttata tgggtataaa tgggctcgcg ataatgtcgg gcaatcaggt |
| 6421 | gcgacaatct atcgcttgta tgggaagccc gatgcgccag agttgtttct gaaacatggc |
| 6481 | aaaggtagcg ttgccaatga tgttacagat gagatggtca gactaaactg gctgacggaa |
| 6541 | tttatgcctc ttccgaccat caagcatttt atccgtactc ctgatgatgc atggttactc |
| 6601 | accactgcga tccccggaaa aacagcattc caggtattag aagaatatcc tgattcaggt |
| 6661 | gaaaatattg ttgatgcgct ggcagtgttc ctgcgccggt tgcattcgat tcctgtttgt |
| 6721 | aattgtcctt ttaacagcga tcgcgtattt cgtctcgctc aggcgcaatc acgaatgaat |
| 6781 | aacggtttgg ttgatgcgag tgattttgat gacgagcgta atggctggcc tgttgaacaa |
| 6841 | gtctggaaag aaatgcataa acttttgcca ttctcaccgg attcagtcgt cactcatggt |
| 6901 | gatttctcac ttgataacct tatttttgac gaggggaaat taataggttg tattgatgtt |
| 6961 | ggacgagtcg gaatcgcaga ccgataccag gatcttgcca tcctatggaa ctgcctcggt |
| 7021 | gagttttctc cttcattaca gaaacggctt tttcaaaaat atggtattga taatcctgat |
| 7081 | atgaataaat tgcagtttca tttgatgctc gatgagtttt tctaagcgta taatggtcta |
| 7141 | gagctagcat atggatccat cgattccatt atacgcctgt cagaccaagt ttactcatat |
| 7201 | atactttaga ttgatttaaa acttcatttt taatttaaaa ggatctaggt gaagatcctt |
| 7261 | tttgataatc tcatgaccaa aatcccttaa cgtgagtttt cgttccactg agcgtcagac |
| 7321 | cccgtagaaa agatcaaagg atcttcttga gatccttttt ttctgcgcgt aatctgctgc |
| 7381 | ttgcaaacaa aaaaaccacc gctaccagcg gtggtttgtt tgccggatca agagctacca |
| 7441 | actctttttc cgaaggtaac tggcttcagc agagcgcaga taccaaatac tgttcttcta |
| 7501 | gtgtagccgt agttaggcca ccacttcaag aactctgtag caccgcctac atacctcgct |
| 7561 | ctgctaatcc tgttaccagt ggctgctgcc agtggcgata agtcgtgtct taccgggttg |
| 7621 | gactcaagac gatagttacc ggataaggcg cagcggtcgg gctgaacggg gggttcgtgc |
| 7681 | acacagccca gcttggagcg aacgacctac accgaactga gatacctaca gcgtgagcta |
| 7741 | tgagaaagcg ccacgcttcc cgaagggaga aaggcggaca ggtatccggt aagcggcagg |
| 7801 | gtcggaacag gagagcgcac gagggagctt ccagggggaa acgcctggta tctttatagt |
| 7861 | cctgtcgggt ttcgccacct ctgacttgag cgtcgatttt tgtgatgctc gtcagggggg |
| 7921 | cggagcctat ggaaaaacgc cagcaacgcg gcctttttac ggttcctggc cttttgctgg |
| 7981 | ccttttgctc acatgt |

| pP067_seq100-IL2wt_2-27 and 32-56 deletion (SEQ ID NO: 472) | |
|---|---|
| (SEQ ID NO: 472) | |
| 1 | tctagagcta gcatatggat ccatcgattt agggataaca gggtaattat cagcacacaa |
| 61 | ttgcccatta tacgcgcgta taatggacta ttgtgtgctg atatctgtac acttaagggc |
| 121 | tagatcttag cttacgtcac tagagggtcc acgtttagtt tttaagatcc attgatctcc |
| 181 | taaacgctgc aagattcgca acctggtata cttagcctag gcgctaggtc ctagtgcagc |
| 241 | gggacttttt ttctaaagtc gttgagagga ggagtcgtca gaccagatag ctttgatgtc |
| 301 | ctgatcggaa ggatcgttgg cccccctgca ggcagctgtt aattaactgc gcgctcgctc |
| 361 | gctcactgag gccgcccggg caaagcccgg gcgtcgggcg acctttggtc gcccggcctc |
| 421 | agtgagcgag cgagcgcgca gagagggagt ggccaactcc atcactaggg gttccttgta |
| 481 | gttaatgatt aacccgccat gctacttatc tacggcgcgc cacgcgttct aggagttaat |
| 541 | ttttaaaaag cagtcaaaag tccaagtgcc cttgcgagca tttactctct ctgtttgctc |
| 601 | tggttaataa tctcaggagc acaaacattc cttactagtt ctaggagtta atttttaaaa |
| 661 | agcagtcaaa agtccaagtg cccttgcgag catttactct ctctgtttgc tctggttaat |
| 721 | aatctcagga gcacaaacat tccttactag ttctagagcg gccgccagtg tgctggaatt |
| 781 | cggctttttt agggctggaa gctacctttg acatcatttc ctctgcgaat gcatgtataa |
| 841 | tttctacaga acctattaga aaggatcacc cagcctctgc ttttgtacaa ctttccctta |
| 901 | aaaaactgcc aatcccactg ctgtttggcc caatagtgag aactttttcc tgctgcctct |
| 961 | tggtgctttt gcctatggcc cctattctgc ctgctgaaga cactcttgcc agcatggact |
| 1021 | taaacccctc cagctctgac aatcctcttt ctcttttgtt ttacatgaag ggtctggcag |
| 1081 | ccaaagcaat cactcaaagt tcaaacctta tcattttttg ctttgttcct cttggccttg |
| 1141 | gttttgtaca tcagctttga aaataccatc ccagggttaa tgctggggtt aatttataac |
| 1201 | tgagagtgct ctagttctgc aatacaggac atgctataaa aatggaaaga tgttgctttc |
| 1261 | tgagagatca ggtggattct tgggcatttg cttaagcttg gtaccttttt tatgggccat |
| 1321 | atcctcatgt acagaatgca gctgctgtcc tgcattgcac tgtcactggc actggttacc |
| 1381 | aactcacagc agggggccag caggccaggc cccagggatg cccaggccca ccctggcaga |
| 1441 | cccagggctg tgcccaccca gtgtgatgtg cctcccaaca gcagatttga ctgtgcccct |
| 1501 | gataaggcca ttactcagga acagtgtgag gccaggggct gctgctatat ccctgccaag |
| 1561 | cagggcctgc agggggccca gatggggcag ccctggtgct tcttcccccc cagctacccc |
| 1621 | tcttataagc tggagaatct gagcagctct gaaatgggct acactgccac tctgaccagg |
| 1681 | actaccccca ccttcttccc caaggatatt ctgactctga ggctggatgt gatgatggag |
| 1741 | actgagaaca ggctgcactt caccatcaag gaccctgcca acaggaggta tgaggtgccc |
| 1801 | ctggaaactc ccagggtgca ctctagggcc cccagccccc tgtattctgt ggagttctct |
| 1861 | gaggagcctt ttggggtcat tgtccacagg cagctggatg gcagggtgct gctgaatact |
| 1921 | actgtggccc ctctgttctt tgctgaccag ttcctgcagc tgagcaccag cctgccttct |
| 1981 | cagtacatta ctggcctggc tgagcatctg agccccctga tgctgagcac ctcttggacc |
| 2041 | agaatcaccc tgtggaacag ggacctggct cccactcctg gggccaacct gtatggcagc |
| 2101 | caccccttct acctggccct ggaggatggg ggctctgccc atggggtgtt cctgctgaac |
| 2161 | agcaatgcca tggatgtggt gctgcagccc tctcctgccc tgtcttggag atctactggg |
| 2221 | ggcatcctgg atgtgtatat cttcctgggg cctgagccca agtctgtggt gcagcagtac |
| 2281 | ctggatgtgg tgggctaccc cttcatgccc ccctactggg gcctgggctt ccacctgtgc |
| 2341 | aggtggggct actcttctac tgctatcacc aggcaggtgg tggagaacat gaccagggct |
| 2401 | cacttccctc tggatgtgca gtggaatgac ctggactaca tggactctag gagagacttc |
| 2461 | acttttaata aggatgggtt cagggacttt cctgccatgg tgcaggagct gcatcagggg |
| 2521 | ggcaggagat atatgatgat tgtggaccct gctatttcta gctctggccc tgctggcagc |
| 2581 | tataggccct atgatgaggg gctgaggagg ggggtgttca tcactaatga gactggccag |
| 2641 | cccctgattg gcaaggtgtg gcctggctct actgccttcc ctgatttcac caaccccact |
| 2701 | gccctggcct ggtgggagga tatggtggct gagtttcatg accaggtgcc ctttgatggc |
| 2761 | atgtggattg acatgaatga gcccagcaac tttatcaggg gctctgaaga tggctgcccc |
| 2821 | aacaatgagc tggagaaccc cccctatgtg cctggggtgg tggggggcac cctgcaggct |
| 2881 | gccaccatct gtgccagcag ccaccagttc ctgagcaccc actataacct gcacaacctg |
| 2941 | tatggcctga ctgaggccat tgcctctcac agggccctgg tgaaggctag ggggactagg |
| 3001 | ccctttgtga tcagcaggtc tacttttgct ggccatggca ggtatgctgg gcactggact |
| 3061 | ggggatgtgt ggtctagctg ggagcagctg gccagctctg tgcctgagat cctgcagttt |
| 3121 | aatctgctgg gggtgcccct ggtgggggct gatgtgtgtg gcttcctggg caatacctct |
| 3181 | gaggagctgt gtgtgaggtg gactcagctg ggggctttct accccttcat gagaaaccac |
| 3241 | aactctctgc tgagcctgcc ccaggagccc tattcttttt ctgagcctgc ccagcaggct |
| 3301 | atgaggaagg ccctgactct gaggtatgcc ctgctgcccc acctgtatac cctgttccat |
| 3361 | caggcccatg tggctgggga gactgtggcc agacctctgt tcctggagtt ccccaaggat |
| 3421 | agctctactt ggactgtgga ccaccagctg ctgtgggggg aggctctgct gatcacccct |
| 3481 | gtgctgcagg ctgggaaggc tgaggtgact ggctatttcc ccctgggcac ctggtatgat |
| 3541 | ctgcagactg tgcccattga ggccctgggc tctctgcccc ctccccctgc tgcccccagg |
| 3601 | gagcctgcca tccactctga gggccagtgg gtgaccctgc ctgcccctct ggacactatc |
| 3661 | aatgtgcacc tgagggctgg ctacatcatc cccctgcagg gccctggcct gactaccact |
| 3721 | gagtctaggc agcagcccat ggccctggct gtggctctga ccaagggggg ggaggccagg |
| 3781 | ggggagctgt tctgggatga tggggagtct ctggaggtgc tggagagggg ggcctacacc |
| 3841 | caggtgatct tcctggctag gaataacacc attgtcaatg agctggtgag ggtgacctct |
| 3901 | gagggggctg gcctgcagct gcagaaggtg actgtgctgg gggtggctac tgccccccag |
| 3961 | caggtgctga gcaatggggt gcctgtgagc aacttcacct atagccctga caccaaggtg |
| 4021 | ctggacatct gtgtgagcct gctgatgggg gagcagttcc tggtgagctg gtgctaattt |
| 4081 | aaatctcgag ccgggcggag tgtgttagtc tctccagagg gaggctggtt ccccagggaa |
| 4141 | gcagagcctg tgtgcgggca gcagctgtgt gcgggcctgg gggttgttaa gtgcaattat |
| 4201 | ttttaataaa aggggcattt ggaaaaaaaa aaaaaaggta gcagtcgaca gatgaattct |
| 4261 | gcagatctgt ggcttctagc tgcccgggtg gcatccctgt gacccctccc cagtgcctct |
| 4321 | cctggccctg gaagttgcca ctccagtgcc caccagcctt gtcctaataa aattaagttg |
| 4381 | catcattttg tctgactagg tgtccttcta taatattatg gggtggaggg gggtggtatg |
| 4441 | gagcaagggg caagttggga agacaacctg tagggcctgc ggggtctatt gggaaccaag |
| 4501 | ctggagtgca gtggcacaat cttggctcac tgcaatctcc gcctcctggg ttcaagcgat |
| 4561 | tctcctgcct cagcctcccg agttgttggg attccaggca tgcatgacca ggctcagcta |
| 4621 | atttttgttt ttttggtaga gacggggttt caccatattg gccaggctgg tctccaactc |
| 4681 | ctaatctcag gtgatctacc caccttggcc tcccaaattg ctgggattac aggcgtgaac |
| 4741 | cactgctccc ttccctgtcc ttctgatttt aaaaaaaaga gaaatgttct ggcacctgca |
| 4801 | cttgcactgg ggacagccta ttttgctagt ttgttttgtt tcgttttgtt ttgatggaga |
| 4861 | gcgtatgttg tttaaacgcg gccgcgtaga taagtagcat ggcgggttaa tcattaacta |
| 4921 | caaggaaccc ctagtgatgg agttggccac tccctctctg cgcgctcgct cgctcactga |
| 4981 | ggccgggcga ccaaaggtcg cccgacgccc gggctttgcc cgggcggcct cagtgagcga |
| 5041 | gcgagcgcgc agttaattaa ggcgccctag gccgaccctt agactctgta ctcagttcta |
| 5101 | taaacgagcc attggatacg agatccgtag attgataagg gacacggaat atccccggac |
| 5161 | gcaatagaca ccggtggaca gcttggtatc ctgagcacag tcgcgcgtcc gaatctagct |
| 5221 | ctactttaga ggccccggat tctgatggtc gtagaccgca gaaccgattg gggggatgag |
| 5281 | atctactagt tatcagcaca caattgccca ttatacgcgc gtataatgga ctattgtgtg |
| 5341 | ctgatatagg gataacaggg taattctaga gctagcatat ggatccatcg atttgatgcg |
| 5401 | gtattttctc cttacgcatc tgtgcggtat ttcacaccgc atacgtcaaa gcaaccatag |
| 5461 | tacgcgccct gtagcggcgc attaagcgcg gcgggtgtgg tggttacgcg cagcgtgacc |
| 5521 | gctacacttg ccagcgccct agcgcccgct cctttcgctt tcttcccttc ctttctcgcc |
| 5581 | acgttcgccg gctttccccg tcaagctcta aatcgggggc tccctttagg gttccgattt |
| 5641 | agtgctttac ggcacctcga ccccaaaaaa cttgatttgg gtgatggttc acgtagtggg |
| 5701 | ccatcgccct gatagacggt ttttcgccct ttgacgttgg agtccacgtt ctttaatagt |
| 5761 | ggactcttgt tccaaactgg aacaacactc aactctatct cgggctattc ttttgattta |
| 5821 | taagggattt tgccgatttc ggtctattgg ttaaaaaatg agctgattta acaaaaattt |
| 5881 | aacgcgaatt ttaacaaaat attaacgttt acaattttat ggtgcactct cagtacaatc |
| 5941 | tgctctgatg ccgcatagtt aagccagccc cgacacccgc caacacccgc tgacgcgccc |
| 6001 | tgacgggctt gtctgctccc ggcatccgct tacagacaag ctgtgaccgt ctccgggagc |
| 6061 | tgcatgtgtc agaggttttc accgtcatca ccgaaacgcg cgagacgaaa gggcctcgtg |
| 6121 | atacgcctat ttttataggt taatgtcatg ataataatgg tttcttagac gtcaggtggc |
| 6181 | acttttcggg gaaatgtgcg cggaacccct atttgtttat ttttctaaat acattcaaat |
| 6241 | atgtatccgc tcatgagaca ataaccctga taaatgcttc aataatattg aaaaaggaag |
| 6301 | agtatgagcc atattcaacg ggaaacgtcg aggccgcgat taaattccaa catggatgct |
| 6361 | gatttatatg ggtataaatg ggctcgcgat aatgtcgggc aatcaggtgc gacaatctat |
| 6421 | cgcttgtatg ggaagcccga tgcgccagag ttgtttctga aacatggcaa aggtagcgtt |
| 6481 | gccaatgatg ttacagatga gatggtcaga ctaaactggc tgacggaatt tatgcctctt |
| 6541 | ccgaccatca agcattttat ccgtactcct gatgatgcat ggttactcac cactgcgatc |
| 6601 | cccggaaaaa cagcattcca ggtattagaa gaatatcctg attcaggtga aaatattgtt |
| 6661 | gatgcgctgg cagtgttcct gcgccggttg cattcgattc ctgtttgtaa ttgtcctttt |
| 6721 | aacagcgatc gcgtatttcg tctcgctcag gcgcaatcac gaatgaataa cggtttggtt |
| 6781 | gatgcgagtg attttgatga cgagcgtaat ggctggcctg ttgaacaagt ctggaaagaa |
| 6841 | atgcataaac ttttgccatt ctcaccggat tcagtcgtca ctcatggtga tttctcactt |
| 6901 | gataacctta tttttgacga ggggaaatta ataggttgta ttgatgttgg acgagtcgga |
| 6961 | atcgcagacc gataccagga tcttgccatc ctatggaact gcctcggtga gttttctcct |
| 7021 | tcattacaga aacggctttt tcaaaaatat ggtattgata atcctgatat gaataaattg |
| 7081 | cagtttcatt tgatgctcga tgagtttttc taagcgtata atggtctaga gctagcatat |
| 7141 | ggatccatcg attccattat acgcctgtca gaccaagttt actcatatat actttagatt |
| 7201 | gatttaaaac ttcattttta atttaaaagg atctaggtga agatcctttt tgataatctc |
| 7261 | atgaccaaaa tcccttaacg tgagttttcg ttccactgag cgtcagaccc cgtagaaaag |
| 7321 | atcaaaggat cttcttgaga tccttttttt ctgcgcgtaa tctgctgctt gcaaacaaaa |
| 7381 | aaaccaccgc taccagcggt ggtttgtttg ccggatcaag agctaccaac tctttttccg |
| 7441 | aaggtaactg gcttcagcag agcgcagata ccaaatactg ttcttctagt gtagccgtag |
| 7501 | ttaggccacc acttcaagaa ctctgtagca ccgcctacat acctcgctct gctaatcctg |
| 7561 | ttaccagtgg ctgctgccag tggcgataag tcgtgtctta ccgggttgga ctcaagacga |
| 7621 | tagttaccgg ataaggcgca gcggtcgggc tgaacggggg gttcgtgcac acagcccagc |
| 7681 | ttggagcgaa cgacctacac cgaactgaga tacctacagc gtgagctatg agaaagcgcc |
| 7741 | acgcttcccg aagggagaaa ggcggacagg tatccggtaa gcggcagggt cggaacagga |
| 7801 | gagcgcacga gggagcttcc agggggaaac gcctggtatc tttatagtcc tgtcgggttt |
| 7861 | cgccacctct gacttgagcg tcgatttttg tgatgctcgt caggggggcg gagcctatgg |
| 7921 | aaaaacgcca gcaacgcggc ctttttacgg ttcctggcct tttgctggcc ttttgctcac |
| 7981 | atgt |

| pP068_seq100-201Ig_2-27 and 23-56 deletion (SEQ ID NO: 473) | |
|---|---|
| (SEQ ID NO: 473) | |
| 1 | tctagagcta gcatatggat ccatcgattt agggataaca gggtaattat cagcacacaa |
| 61 | ttgcccatta tacgcgcgta taatggacta ttgtgtgctg atatctgtac acttaagggc |
| 121 | tagatcttag cttacgtcac tagagggtcc acgtttagtt tttaagatcc attgatctcc |
| 181 | taaacgctgc aagattcgca acctggtata cttagcctag gcgctaggtc ctagtgcagc |
| 241 | gggacttttt ttctaaagtc gttgagagga ggagtcgtca gaccagatag ctttgatgtc |
| 301 | ctgatcggaa ggatcgttgg cccccctgca ggcagctgtt aattaactgc gcgctcgctc |
| 361 | gctcactgag gccgcccggg caaagcccgg gcgtcgggcg acctttggtc gcccggcctc |
| 421 | agtgagcgag cgagcgcgca gagagggagt ggccaactcc atcactaggg gttccttgta |
| 481 | gttaatgatt aacccgccat gctacttatc tacggcgcgc cacgcgttct aggagttaat |
| 541 | ttttaaaaag cagtcaaaag tccaagtgcc cttgcgagca tttactctct ctgtttgctc |
| 601 | tggttaataa tctcaggagc acaaacattc cttactagtt ctaggagtta atttttaaaa |
| 661 | agcagtcaaa agtccaagtg cccttgcgag catttactct ctctgtttgc tctggttaat |
| 721 | aatctcagga gcacaaacat tccttactag ttctagagcg gccgccagtg tgctggaatt |
| 781 | cggctttttt agggctggaa gctacctttg acatcatttc ctctgcgaat gcatgtataa |
| 841 | tttctacaga acctattaga aaggatcacc cagcctctgc ttttgtacaa ctttccctta |
| 901 | aaaaactgcc aatcccactg ctgtttggcc caatagtgag aactttttcc tgctgcctct |
| 961 | tggtgctttt gcctatggcc cctattctgc ctgctgaaga cactcttgcc agcatggact |
| 1021 | taaacccctc cagctctgac aatcctcttt ctcttttgtt ttacatgaag ggtctggcag |
| 1081 | ccaaagcaat cactcaaagt tcaaacctta tcattttttg ctttgttcct cttggccttg |
| 1141 | gttttgtaca tcagctttga aaataccatc ccagggttaa tgctggggtt aatttataac |
| 1201 | tgagagtgct ctagttctgc aatacaggac atgctataaa aatggaaaga tgttgctttc |
| 1261 | tgagagatca ggtggattct tgggcatttg cttaagcttg gtaccttttt tatgggccat |
| 1321 | atcctcatgg agtttgggct gagctgggtc tttctggtgg ccctgctgaa gggagtccag |
| 1381 | tgtgagcagc agggggccag caggccaggc cccagggatg cccaggccca ccctggcaga |
| 1441 | cccagggctg tgcccaccca gtgtgatgtg cctcccaaca gcagatttga ctgtgcccct |
| 1501 | gataaggcca ttactcagga acagtgtgag gccaggggct gctgctatat ccctgccaag |
| 1561 | cagggcctgc agggggccca gatggggcag ccctggtgct tcttcccccc cagctacccc |
| 1621 | tcttataagc tggagaatct gagcagctct gaaatgggct acactgccac tctgaccagg |
| 1681 | actaccccca ccttcttccc caaggatatt ctgactctga ggctggatgt gatgatggag |
| 1741 | actgagaaca ggctgcactt caccatcaag gaccctgcca acaggaggta tgaggtgccc |
| 1801 | ctggaaactc ccagggtgca ctctagggcc cccagccccc tgtattctgt ggagttctct |
| 1861 | gaggagcctt ttggggtcat tgtccacagg cagctggatg gcagggtgct gctgaatact |
| 1921 | actgtggccc ctctgttctt tgctgaccag ttcctgcagc tgagcaccag cctgccttct |
| 1981 | cagtacatta ctggcctggc tgagcatctg agccccctga tgctgagcac ctcttggacc |
| 2041 | agaatcaccc tgtggaacag ggacctggct cccactcctg gggccaacct gtatggcagc |
| 2101 | caccccttct acctggccct ggaggatggg ggctctgccc atggggtgtt cctgctgaac |
| 2161 | agcaatgcca tggatgtggt gctgcagccc tctcctgccc tgtcttggag atctactggg |
| 2221 | ggcatcctgg atgtgtatat cttcctgggg cctgagccca agtctgtggt gcagcagtac |
| 2281 | ctggatgtgg tgggctaccc cttcatgccc ccctactggg gcctgggctt ccacctgtgc |
| 2341 | aggtggggct actcttctac tgctatcacc aggcaggtgg tggagaacat gaccagggct |
| 2401 | cacttccctc tggatgtgca gtggaatgac ctggactaca tggactctag gagagacttc |
| 2461 | acttttaata aggatgggtt cagggacttt cctgccatgg tgcaggagct gcatcagggg |
| 2521 | ggcaggagat atatgatgat tgtggaccct gctatttcta gctctggccc tgctggcagc |
| 2581 | tataggccct atgatgaggg gctgaggagg ggggtgttca tcactaatga gactggccag |
| 2641 | cccctgattg gcaaggtgtg gcctggctct actgccttcc ctgatttcac caaccccact |
| 2701 | gccctggcct ggtgggagga tatggtggct gagtttcatg accaggtgcc ctttgatggc |
| 2761 | atgtggattg acatgaatga gcccagcaac tttatcaggg gctctgaaga tggctgcccc |
| 2821 | aacaatgagc tggagaaccc cccctatgtg cctggggtgg tggggggcac cctgcaggct |
| 2881 | gccaccatct gtgccagcag ccaccagttc ctgagcaccc actataacct gcacaacctg |
| 2941 | tatggcctga ctgaggccat tgcctctcac agggccctgg tgaaggctag ggggactagg |
| 3001 | ccctttgtga tcagcaggtc tacttttgct ggccatggca ggtatgctgg gcactggact |
| 3061 | ggggatgtgt ggtctagctg ggagcagctg gccagctctg tgcctgagat cctgcagttt |
| 3121 | aatctgctgg gggtgcccct ggtgggggct gatgtgtgtg gcttcctggg caatacctct |
| 3181 | gaggagctgt gtgtgaggtg gactcagctg ggggctttct accccttcat gagaaaccac |
| 3241 | aactctctgc tgagcctgcc ccaggagccc tattcttttt ctgagcctgc ccagcaggct |
| 3301 | atgaggaagg ccctgactct gaggtatgcc ctgctgcccc acctgtatac cctgttccat |
| 3361 | caggcccatg tggctgggga gactgtggcc agacctctgt tcctggagtt ccccaaggat |
| 3421 | agctctactt ggactgtgga ccaccagctg ctgtgggggg aggctctgct gatcacccct |
| 3481 | gtgctgcagg ctgggaaggc tgaggtgact ggctatttcc ccctgggcac ctggtatgat |
| 3541 | ctgcagactg tgcccattga ggccctgggc tctctgcccc ctccccctgc tgcccccagg |
| 3601 | gagcctgcca tccactctga gggccagtgg gtgaccctgc ctgcccctct ggacactatc |
| 3661 | aatgtgcacc tgagggctgg ctacatcatc cccctgcagg gccctggcct gactaccact |
| 3721 | gagtctaggc agcagcccat ggccctggct gtggctctga ccaagggggg ggaggccagg |
| 3781 | ggggagctgt tctgggatga tggggagtct ctggaggtgc tggagagggg ggcctacacc |
| 3841 | caggtgatct tcctggctag gaataacacc attgtcaatg agctggtgag ggtgacctct |
| 3901 | gagggggctg gcctgcagct gcagaaggtg actgtgctgg gggtggctac tgccccccag |
| 3961 | caggtgctga gcaatggggt gcctgtgagc aacttcacct atagccctga caccaaggtg |
| 4021 | ctggacatct gtgtgagcct gctgatgggg gagcagttcc tggtgagctg gtgctaattt |
| 4081 | aaatctcgag ccgggcggag tgtgttagtc tctccagagg gaggctggtt ccccagggaa |
| 4141 | gcagagcctg tgtgcgggca gcagctgtgt gcgggcctgg gggttgttaa gtgcaattat |
| 4201 | ttttaataaa aggggcattt ggaaaaaaaa aaaaaaggta gcagtcgaca gatgaattct |
| 4261 | gcagatctgt ggcttctagc tgcccgggtg gcatccctgt gacccctccc cagtgcctct |
| 4321 | cctggccctg gaagttgcca ctccagtgcc caccagcctt gtcctaataa aattaagttg |
| 4381 | catcattttg tctgactagg tgtccttcta taatattatg gggtggaggg gggtggtatg |
| 4441 | gagcaagggg caagttggga agacaacctg tagggcctgc ggggtctatt gggaaccaag |
| 4501 | ctggagtgca gtggcacaat cttggctcac tgcaatctcc gcctcctggg ttcaagcgat |
| 4561 | tctcctgcct cagcctcccg agttgttggg attccaggca tgcatgacca ggctcagcta |
| 4621 | atttttgttt ttttggtaga gacggggttt caccatattg gccaggctgg tctccaactc |
| 4681 | ctaatctcag gtgatctacc caccttggcc tcccaaattg ctgggattac aggcgtgaac |
| 4741 | cactgctccc ttccctgtcc ttctgatttt aaaaaaaaga gaaatgttct ggcacctgca |
| 4801 | cttgcactgg ggacagccta ttttgctagt ttgttttgtt tcgttttgtt ttgatggaga |
| 4861 | gcgtatgttg tttaaacgcg gccgcgtaga taagtagcat ggcgggttaa tcattaacta |
| 4921 | caaggaaccc ctagtgatgg agttggccac tccctctctg cgcgctcgct cgctcactga |
| 4981 | ggccgggcga ccaaaggtcg cccgacgccc gggctttgcc cgggcggcct cagtgagcga |
| 5041 | gcgagcgcgc agttaattaa ggcgccctag gccgaccctt agactctgta ctcagttcta |
| 5101 | taaacgagcc attggatacg agatccgtag attgataagg gacacggaat atccccggac |
| 5161 | gcaatagaca ccggtggaca gcttggtatc ctgagcacag tcgcgcgtcc gaatctagct |
| 5221 | ctactttaga ggccccggat tctgatggtc gtagaccgca gaaccgattg gggggatgag |
| 5281 | atctactagt tatcagcaca caattgccca ttatacgcgc gtataatgga ctattgtgtg |
| 5341 | ctgatatagg gataacaggg taattctaga gctagcatat ggatccatcg atttgatgcg |
| 5401 | gtattttctc cttacgcatc tgtgcggtat ttcacaccgc atacgtcaaa gcaaccatag |
| 5461 | tacgcgccct gtagcggcgc attaagcgcg gcgggtgtgg tggttacgcg cagcgtgacc |
| 5521 | gctacacttg ccagcgccct agcgcccgct cctttcgctt tcttcccttc ctttctcgcc |
| 5581 | acgttcgccg gctttccccg tcaagctcta aatcgggggc tccctttagg gttccgattt |
| 5641 | agtgctttac ggcacctcga ccccaaaaaa cttgatttgg gtgatggttc acgtagtggg |
| 5701 | ccatcgccct gatagacggt ttttcgccct ttgacgttgg agtccacgtt ctttaatagt |
| 5761 | ggactcttgt tccaaactgg aacaacactc aactctatct cgggctattc ttttgattta |
| 5821 | taagggattt tgccgatttc ggtctattgg ttaaaaaatg agctgattta acaaaaattt |
| 5881 | aacgcgaatt ttaacaaaat attaacgttt acaattttat ggtgcactct cagtacaatc |
| 5941 | tgctctgatg ccgcatagtt aagccagccc cgacacccgc caacacccgc tgacgcgccc |
| 6001 | tgacgggctt gtctgctccc ggcatccgct tacagacaag ctgtgaccgt ctccgggagc |
| 6061 | tgcatgtgtc agaggttttc accgtcatca ccgaaacgcg cgagacgaaa gggcctcgtg |
| 6121 | atacgcctat ttttataggt taatgtcatg ataataatgg tttcttagac gtcaggtggc |
| 6181 | acttttcggg gaaatgtgcg cggaacccct atttgtttat ttttctaaat acattcaaat |
| 6241 | atgtatccgc tcatgagaca ataaccctga taaatgcttc aataatattg aaaaaggaag |
| 6301 | agtatgagcc atattcaacg ggaaacgtcg aggccgcgat taaattccaa catggatgct |
| 6361 | gatttatatg ggtataaatg ggctcgcgat aatgtcgggc aatcaggtgc gacaatctat |
| 6421 | cgcttgtatg ggaagcccga tgcgccagag ttgtttctga aacatggcaa aggtagcgtt |
| 6481 | gccaatgatg ttacagatga gatggtcaga ctaaactggc tgacggaatt tatgcctctt |
| 6541 | ccgaccatca agcattttat ccgtactcct gatgatgcat ggttactcac cactgcgatc |
| 6601 | cccggaaaaa cagcattcca ggtattagaa gaatatcctg attcaggtga aaatattgtt |
| 6661 | gatgcgctgg cagtgttcct gcgccggttg cattcgattc ctgtttgtaa ttgtcctttt |
| 6721 | aacagcgatc gcgtatttcg tctcgctcag gcgcaatcac gaatgaataa cggtttggtt |
| 6781 | gatgcgagtg attttgatga cgagcgtaat ggctggcctg ttgaacaagt ctggaaagaa |
| 6841 | atgcataaac ttttgccatt ctcaccggat tcagtcgtca ctcatggtga tttctcactt |
| 6901 | gataacctta tttttgacga ggggaaatta ataggttgta ttgatgttgg acgagtcgga |
| 6961 | atcgcagacc gataccagga tcttgccatc ctatggaact gcctcggtga gttttctcct |
| 7021 | tcattacaga aacggctttt tcaaaaatat ggtattgata atcctgatat gaataaattg |
| 7081 | cagtttcatt tgatgctcga tgagtttttc taagcgtata atggtctaga gctagcatat |
| 7141 | ggatccatcg attccattat acgcctgtca gaccaagttt actcatatat actttagatt |
| 7201 | gatttaaaac ttcattttta atttaaaagg atctaggtga agatcctttt tgataatctc |
| 7261 | atgaccaaaa tcccttaacg tgagttttcg ttccactgag cgtcagaccc cgtagaaaag |
| 7321 | atcaaaggat cttcttgaga tccttttttt ctgcgcgtaa tctgctgctt gcaaacaaaa |
| 7381 | aaaccaccgc taccagcggt ggtttgtttg ccggatcaag agctaccaac tctttttccg |
| 7441 | aaggtaactg gcttcagcag agcgcagata ccaaatactg ttcttctagt gtagccgtag |
| 7501 | ttaggccacc acttcaagaa ctctgtagca ccgcctacat acctcgctct gctaatcctg |
| 7561 | ttaccagtgg ctgctgccag tggcgataag tcgtgtctta ccgggttgga ctcaagacga |
| 7621 | tagttaccgg ataaggcgca gcggtcgggc tgaacggggg gttcgtgcac acagcccagc |
| 7681 | ttggagcgaa cgacctacac cgaactgaga tacctacagc gtgagctatg agaaagcgcc |
| 7741 | acgcttcccg aagggagaaa ggcggacagg tatccggtaa gcggcagggt cggaacagga |
| 7801 | gagcgcacga gggagcttcc agggggaaac gcctggtatc tttatagtcc tgtcgggttt |
| 7861 | cgccacctct gacttgagcg tcgatttttg tgatgctcgt caggggggcg gagcctatgg |
| 7921 | aaaaacgcca gcaacgcggc ctttttacgg ttcctggcct tttgctggcc ttttgctcac |
| 7981 | atgt |

| pP069_seq3-IL2wt 2-27 and 32-56 deletion | |
|---|---|
| (SEQ ID NO: 474) | |
| 1 | tctagagcta gcatatggat ccatcgattt agggataaca gggtaattat cagcacacaa |
| 61 | ttgcccatta tacgcgcgta taatggacta ttgtgtgctg atatctgtac acttaagggc |
| 121 | tagatcttag cttacgtcac tagagggtcc acgtttagtt tttaagatcc attgatctcc |
| 181 | taaacgctgc aagattcgca acctggtata cttagcctag gcgctaggtc ctagtgcagc |
| 241 | gggacttttt ttctaaagtc gttgagagga ggagtcgtca gaccagatag ctttgatgtc |
| 301 | ctgatcggaa ggatcgttgg cccccctgca ggcagctgtt aattaactgc gcgctcgctc |
| 361 | gctcactgag gccgcccggg caaagcccgg gcgtcgggcg acctttggtc gcccggcctc |
| 421 | agtgagcgag cgagcgcgca gagagggagt ggccaactcc atcactaggg gttccttgta |
| 481 | gttaatgatt aacccgccat gctacttatc tacggcgcgc cacgcgttct aggagttaat |
| 541 | ttttaaaaag cagtcaaaag tccaagtgcc cttgcgagca tttactctct ctgtttgctc |
| 601 | tggttaataa tctcaggagc acaaacattc cttactagtt ctaggagtta atttttaaaa |
| 661 | agcagtcaaa agtccaagtg cccttgcgag catttactct ctctgtttgc tctggttaat |
| 721 | aatctcagga gcacaaacat tccttactag ttctagagcg gccgccagtg tgctggaatt |
| 781 | cggctttttt agggctggaa gctacctttg acatcatttc ctctgcgaat gcatgtataa |
| 841 | tttctacaga acctattaga aaggatcacc cagcctctgc ttttgtacaa ctttccctta |
| 901 | aaaaactgcc aatcccactg ctgtttggcc caatagtgag aactttttcc tgctgcctct |
| 961 | tggtgctttt gcctatggcc cctattctgc ctgctgaaga cactcttgcc agcatggact |
| 1021 | taaacccctc cagctctgac aatcctcttt ctcttttgtt ttacatgaag ggtctggcag |
| 1081 | ccaaagcaat cactcaaagt tcaaacctta tcattttttg ctttgttcct cttggccttg |
| 1141 | gttttgtaca tcagctttga aaataccatc ccagggttaa tgctggggtt aatttataac |
| 1201 | tgagagtgct ctagttctgc aatacaggac atgctataaa aatggaaaga tgttgctttc |
| 1261 | tgagagatca ggtggattct tgggcatttg cttaagcttg gtaccttttt tatgggacat |
| 1321 | attctcatgt acagaatgca gctgctgtcc tgcattgcac tgtcactggc actggttacc |
| 1381 | aactcacagc agggagcttc tagacctgga cctagagatg ctcaggctca tcctggaaga |
| 1441 | cctagagctg tgcctacaca gtgtgatgtg cctcctaatt ctagatttga ttgtgctcct |
| 1501 | gataaggcta taacacagga gcagtgtgag gctagaggat gttgttatat tcctgctaag |
| 1561 | cagggactgc agggagctca gatgggacag ccttggtgtt tttttcctcc ttcttatcct |
| 1621 | tcttataagc tggagaatct gtcttcttct gagatgggat atacagctac actgacaaga |
| 1681 | acaacaccta cattttttcc taaggatatt ctgacactga gactggatgt gatgatggag |
| 1741 | acagagaata gactgcattt tacaattaag gatcctgcta atagaagata tgaggtgcct |
| 1801 | ctagagacac ctagagtgca ttctagagct ccttctcctc tgtattctgt ggagttttct |
| 1861 | gaggagcctt ttggagtgat tgtgcataga cagctggatg gaagagtgct gctgaataca |
| 1921 | acagtggctc ctctgttttt tgctgatcag tttctgcagc tgtctacatc tctgccttct |
| 1981 | cagtatatta caggactggc tgagcatctg tctcctctga tgctgtctac atcttggaca |
| 2041 | agaattacac tgtggaatag agatctggct cctacacctg gagctaatct gtatggatct |
| 2101 | catccttttt atctggctct ggaggatgga ggatctgctc atggagtgtt tctgctgaat |
| 2161 | tctaatgcta tggatgtagt gctgcagcct tctcctgctc tgtcttggag atctacagga |
| 2221 | ggaattctgg atgtgtatat ttttctagga cctgagccta agtctgtggt gcagcagtat |
| 2281 | ctggatgtag tgggatatcc ttttatgcct ccttattggg gactgggatt tcatctgtgt |
| 2341 | agatggggat attcttctac agctattaca agacaggtgg ttgagaatat gacaagagct |
| 2401 | cattttcctc tggatgtgca gtggaatgat ctagattata tggattctag aagagatttt |
| 2461 | acatttaata aggatggatt tagagatttt cctgctatgg tgcaggagct gcatcaggga |
| 2521 | ggaagaagat atatgatgat tgtggatcct gctatttctt cttctggacc tgctggatct |
| 2581 | tatagacctt atgatgaggg actgagaaga ggagtgttta ttacaaatga gacaggacag |
| 2641 | cctctgattg gaaaggtgtg gcctggatct acagcttttc ctgattttac aaatcctaca |
| 2701 | gctctggctt ggtgggagga tatggtggct gagtttcatg atcaggtgcc ttttgatgga |
| 2761 | atgtggattg atatgaatga gccttctaat tttataagag gatctgagga tggatgtcct |
| 2821 | aataatgagc tagagaatcc tccttatgtg cctggagtag tgggaggaac actgcaggct |
| 2881 | gctacaattt gtgcttcttc tcatcagttt ctgtctacac attataatct gcataatctg |
| 2941 | tatggactga cagaggctat tgcttctcat agagctctgg tgaaggctag aggaacaaga |
| 3001 | ccttttgtga tttctagatc tacatttgct ggacatggaa gatatgctgg acattggaca |
| 3061 | ggagatgtat ggtcttcttg ggagcagcta gcttcttctg tgcctgagat tctgcagttt |
| 3121 | aatctgctag gagtgcctct ggtgggagct gatgtgtgtg gatttctggg aaatacatct |
| 3181 | gaggagctgt gtgtgagatg gacacagctg ggagcttttt atccttttat gagaaatcat |
| 3241 | aattctctgc tgtctctgcc tcaggagcct tattcttttt ctgagcctgc tcagcaggct |
| 3301 | atgagaaagg ctctgacact gagatatgct ctgctgcctc atctgtatac actgtttcat |
| 3361 | caggctcatg tggctggaga gacagtagct agacctctgt ttctggagtt tcctaaggat |
| 3421 | tcttctacat ggacagttga tcatcagctg ctgtggggag aggctctgct gattacacct |
| 3481 | gtgctgcagg ctggaaaggc tgaggtgaca ggatattttc ctctgggaac atggtatgat |
| 3541 | ctgcagacag tgcctataga ggctctggga tctctgcctc ctcctcctgc tgctcctaga |
| 3601 | gagcctgcta ttcattctga gggacagtgg gtgacactgc ctgctcctct ggatacaatt |
| 3661 | aatgtgcatc tgagagctgg atatattatt cctctgcagg gacctggact gacaacaaca |
| 3721 | gagtctagac agcagcctat ggctctggct gtggctctga caaagggagg agaggctaga |
| 3781 | ggagagctgt tttgggatga tggagagtct ctggaggtgc tggagagagg agcttataca |
| 3841 | caggtgattt ttctggctag aaataataca attgtgaatg agctggtgag agtgacatct |
| 3901 | gagggagctg gactgcagct gcagaaggtg acagtgctgg gagtggctac agctcctcag |
| 3961 | caggtgctgt ctaatggagt gcctgtgtct aattttacat attctcctga tacaaaggtg |
| 4021 | ctggatattt gtgtgtctct gctgatggga gagcagtttc tggtgtcttg gtgttaatga |
| 4081 | tttaaatctc gagccgggcg gagtgtgtta gtctctccag agggaggctg gttccccagg |
| 4141 | gaagcagagc ctgtgtgcgg gcagcagctg tgtgcgggcc tgggggttgt taagtgcaat |
| 4201 | tatttttaat aaaaggggca tttggaaaaa aaaaaaaaag gtagcagtcg acagatgaat |
| 4261 | tctgcagatc tgtggcttct agctgcccgg gtggcatccc tgtgacccct ccccagtgcc |
| 4321 | tctcctggcc ctggaagttg ccactccagt gcccaccagc cttgtcctaa taaaattaag |
| 4381 | ttgcatcatt ttgtctgact aggtgtcctt ctataatatt atggggtgga ggggggtggt |
| 4441 | atggagcaag gggcaagttg ggaagacaac ctgtagggcc tgcggggtct attgggaacc |
| 4501 | aagctggagt gcagtggcac aatcttggct cactgcaatc tccgcctcct gggttcaagc |
| 4561 | gattctcctg cctcagcctc ccgagttgtt gggattccag gcatgcatga ccaggctcag |
| 4621 | ctaatttttg tttttttggt agagacgggg tttcaccata ttggccaggc tggtctccaa |
| 4681 | ctcctaatct caggtgatct acccaccttg gcctcccaaa ttgctgggat tacaggcgtg |
| 4741 | aaccactgct cccttccctg tccttctgat tttaaaaaaa agagaaatgt tctggcacct |
| 4801 | gcacttgcac tggggacagc ctattttgct agtttgtttt gtttcgtttt gttttgatgg |
| 4861 | agagcgtatg ttgtttaaac gcggccgcgt agataagtag catggcgggt taatcattaa |
| 4921 | ctacaaggaa cccctagtga tggagttggc cactccctct ctgcgcgctc gctcgctcac |
| 4981 | tgaggccggg cgaccaaagg tcgcccgacg cccgggcttt gcccgggcgg cctcagtgag |
| 5041 | cgagcgagcg cgcagttaat taaggcgccc taggccgacc cttagactct gtactcagtt |
| 5101 | ctataaacga gccattggat acgagatccg tagattgata agggacacgg aatatccccg |
| 5161 | gacgcaatag acaccggtgg acagcttggt atcctgagca cagtcgcgcg tccgaatcta |
| 5221 | gctctacttt agaggccccg gattctgatg gtcgtagacc gcagaaccga ttggggggat |
| 5281 | gagatctact agttatcagc acacaattgc ccattatacg cgcgtataat ggactattgt |
| 5341 | gtgctgatat agggataaca gggtaattct agagctagca tatggatcca tcgatttgat |
| 5401 | gcggtatttt ctccttacgc atctgtgcgg tatttcacac cgcatacgtc aaagcaacca |
| 5461 | tagtacgcgc cctgtagcgg cgcattaagc gcggcgggtg tggtggttac gcgcagcgtg |
| 5521 | accgctacac ttgccagcgc cctagcgccc gctcctttcg ctttcttccc ttcctttctc |
| 5581 | gccacgttcg ccggctttcc ccgtcaagct ctaaatcggg ggctcccttt agggttccga |
| 5641 | tttagtgctt tacggcacct cgaccccaaa aaacttgatt tgggtgatgg ttcacgtagt |
| 5701 | gggccatcgc cctgatagac ggtttttcgc cctttgacgt tggagtccac gttctttaat |
| 5761 | agtggactct tgttccaaac tggaacaaca ctcaactcta tctcgggcta ttcttttgat |
| 5821 | ttataaggga ttttgccgat ttcggtctat tggttaaaaa atgagctgat ttaacaaaaa |
| 5881 | tttaacgcga attttaacaa aatattaacg tttacaattt tatggtgcac tctcagtaca |
| 5941 | atctgctctg atgccgcata gttaagccag ccccgacacc cgccaacacc cgctgacgcg |
| 6001 | ccctgacggg cttgtctgct cccggcatcc gcttacagac aagctgtgac cgtctccggg |
| 6061 | agctgcatgt gtcagaggtt ttcaccgtca tcaccgaaac gcgcgagacg aaagggcctc |
| 6121 | gtgatacgcc tatttttata ggttaatgtc atgataataa tggtttctta gacgtcaggt |
| 6181 | ggcacttttc ggggaaatgt gcgcggaacc cctatttgtt tatttttcta aatacattca |
| 6241 | aatatgtatc cgctcatgag acaataaccc tgataaatgc ttcaataata ttgaaaaagg |
| 6301 | aagagtatga gccatattca acgggaaacg tcgaggccgc gattaaattc caacatggat |
| 6361 | gctgatttat atgggtataa atgggctcgc gataatgtcg ggcaatcagg tgcgacaatc |
| 6421 | tatcgcttgt atgggaagcc cgatgcgcca gagttgtttc tgaaacatgg caaaggtagc |
| 6481 | gttgccaatg atgttacaga tgagatggtc agactaaact ggctgacgga atttatgcct |
| 6541 | cttccgacca tcaagcattt tatccgtact cctgatgatg catggttact caccactgcg |
| 6601 | atccccggaa aaacagcatt ccaggtatta gaagaatatc ctgattcagg tgaaaatatt |
| 6661 | gttgatgcgc tggcagtgtt cctgcgccgg ttgcattcga ttcctgtttg taattgtcct |
| 6721 | tttaacagcg atcgcgtatt tcgtctcgct caggcgcaat cacgaatgaa taacggtttg |
| 6781 | gttgatgcga gtgattttga tgacgagcgt aatggctggc ctgttgaaca agtctggaaa |
| 6841 | gaaatgcata aacttttgcc attctcaccg gattcagtcg tcactcatgg tgatttctca |
| 6901 | cttgataacc ttatttttga cgaggggaaa ttaataggtt gtattgatgt tggacgagtc |
| 6961 | ggaatcgcag accgatacca ggatcttgcc atcctatgga actgcctcgg tgagttttct |
| 7021 | ccttcattac agaaacggct ttttcaaaaa tatggtattg ataatcctga tatgaataaa |
| 7081 | ttgcagtttc atttgatgct cgatgagttt ttctaagcgt ataatggtct agagctagca |
| 7141 | tatggatcca tcgattccat tatacgcctg tcagaccaag tttactcata tatactttag |
| 7201 | attgatttaa aacttcattt ttaatttaaa aggatctagg tgaagatcct ttttgataat |
| 7261 | ctcatgacca aaatccctta acgtgagttt tcgttccact gagcgtcaga ccccgtagaa |
| 7321 | aagatcaaag gatcttcttg agatcctttt tttctgcgcg taatctgctg cttgcaaaca |
| 7381 | aaaaaaccac cgctaccagc ggtggtttgt ttgccggatc aagagctacc aactcttttt |
| 7441 | ccgaaggtaa ctggcttcag cagagcgcag ataccaaata ctgttcttct agtgtagccg |
| 7501 | tagttaggcc accacttcaa gaactctgta gcaccgccta catacctcgc tctgctaatc |
| 7561 | ctgttaccag tggctgctgc cagtggcgat aagtcgtgtc ttaccgggtt ggactcaaga |
| 7621 | cgatagttac cggataaggc gcagcggtcg ggctgaacgg ggggttcgtg cacacagccc |
| 7681 | agcttggagc gaacgaccta caccgaactg agatacctac agcgtgagct atgagaaagc |
| 7741 | gccacgcttc ccgaagggag aaaggcggac aggtatccgg taagcggcag ggtcggaaca |
| 7801 | ggagagcgca cgagggagct tccaggggga aacgcctggt atctttatag tcctgtcggg |
| 7861 | tttcgccacc tctgacttga gcgtcgattt ttgtgatgct cgtcaggggg gcggagccta |
| 7921 | tggaaaaacg ccagcaacgc ggccttttta cggttcctgg ccttttgctg gccttttgct |
| 7981 | cacatgt |

| pP070_seq3-201Ig_2-27 and 32-56 deletion (SEQ ID NO: 475) | |
|---|---|
| (SEQ ID NO: 475) | |
| 1 | tctagagcta gcatatggat ccatcgattt agggataaca gggtaattat cagcacacaa |
| 61 | ttgcccatta tacgcgcgta taatggacta ttgtgtgctg atatctgtac acttaagggc |
| 121 | tagatcttag cttacgtcac tagagggtcc acgtttagtt tttaagatcc attgatctcc |
| 181 | taaacgctgc aagattcgca acctggtata cttagcctag gcgctaggtc ctagtgcagc |
| 241 | gggacttttt ttctaaagtc gttgagagga ggagtcgtca gaccagatag ctttgatgtc |
| 301 | ctgatcggaa ggatcgttgg cccccctgca ggcagctgtt aattaactgc gcgctcgctc |
| 361 | gctcactgag gccgcccggg caaagcccgg gcgtcgggcg acctttggtc gcccggcctc |
| 421 | agtgagcgag cgagcgcgca gagagggagt ggccaactcc atcactaggg gttccttgta |
| 481 | gttaatgatt aacccgccat gctacttatc tacggcgcgc cacgcgttct aggagttaat |
| 541 | ttttaaaaag cagtcaaaag tccaagtgcc cttgcgagca tttactctct ctgtttgctc |
| 601 | tggttaataa tctcaggagc acaaacattc cttactagtt ctaggagtta atttttaaaa |
| 661 | agcagtcaaa agtccaagtg cccttgcgag catttactct ctctgtttgc tctggttaat |
| 721 | aatctcagga gcacaaacat tccttactag ttctagagcg gccgccagtg tgctggaatt |
| 781 | cggctttttt agggctggaa gctacctttg acatcatttc ctctgcgaat gcatgtataa |
| 841 | tttctacaga acctattaga aaggatcacc cagcctctgc ttttgtacaa ctttccctta |
| 901 | aaaaactgcc aatcccactg ctgtttggcc caatagtgag aactttttcc tgctgcctct |
| 961 | tggtgctttt gcctatggcc cctattctgc ctgctgaaga cactcttgcc agcatggact |
| 1021 | taaacccctc cagctctgac aatcctcttt ctcttttgtt ttacatgaag ggtctggcag |
| 1081 | ccaaagcaat cactcaaagt tcaaacctta tcattttttg ctttgttcct cttggccttg |
| 1141 | gttttgtaca tcagctttga aaataccatc ccagggttaa tgctggggtt aatttataac |
| 1201 | tgagagtgct ctagttctgc aatacaggac atgctataaa aatggaaaga tgttgctttc |
| 1261 | tgagagatca ggtggattct tgggcatttg cttaagcttg gtaccttttt tatgggacat |
| 1321 | attctcatgg agtttgggct gagctgggtc tttctggtgg ccctgctgaa gggagtccag |
| 1381 | tgtgagcagc agggagcttc tagacctgga cctagagatg ctcaggctca tcctggaaga |
| 1441 | cctagagctg tgcctacaca gtgtgatgtg cctcctaatt ctagatttga ttgtgctcct |
| 1501 | gataaggcta taacacagga gcagtgtgag gctagaggat gttgttatat tcctgctaag |
| 1561 | cagggactgc agggagctca gatgggacag ccttggtgtt tttttcctcc ttcttatcct |
| 1621 | tcttataagc tggagaatct gtcttcttct gagatgggat atacagctac actgacaaga |
| 1681 | acaacaccta cattttttcc taaggatatt ctgacactga gactggatgt gatgatggag |
| 1741 | acagagaata gactgcattt tacaattaag gatcctgcta atagaagata tgaggtgcct |
| 1801 | ctagagacac ctagagtgca ttctagagct ccttctcctc tgtattctgt ggagttttct |
| 1861 | gaggagcctt ttggagtgat tgtgcataga cagctggatg gaagagtgct gctgaataca |
| 1921 | acagtggctc ctctgttttt tgctgatcag tttctgcagc tgtctacatc tctgccttct |
| 1981 | cagtatatta caggactggc tgagcatctg tctcctctga tgctgtctac atcttggaca |
| 2041 | agaattacac tgtggaatag agatctggct cctacacctg gagctaatct gtatggatct |
| 2101 | catccttttt atctggctct ggaggatgga ggatctgctc atggagtgtt tctgctgaat |
| 2161 | tctaatgcta tggatgtagt gctgcagcct tctcctgctc tgtcttggag atctacagga |
| 2221 | ggaattctgg atgtgtatat ttttctagga cctgagccta agtctgtggt gcagcagtat |
| 2281 | ctggatgtag tgggatatcc ttttatgcct ccttattggg gactgggatt tcatctgtgt |
| 2341 | agatggggat attcttctac agctattaca agacaggtgg ttgagaatat gacaagagct |
| 2401 | cattttcctc tggatgtgca gtggaatgat ctagattata tggattctag aagagatttt |
| 2461 | acatttaata aggatggatt tagagatttt cctgctatgg tgcaggagct gcatcaggga |
| 2521 | ggaagaagat atatgatgat tgtggatcct gctatttctt cttctggacc tgctggatct |
| 2581 | tatagacctt atgatgaggg actgagaaga ggagtgttta ttacaaatga gacaggacag |
| 2641 | cctctgattg gaaaggtgtg gcctggatct acagcttttc ctgattttac aaatcctaca |
| 2701 | gctctggctt ggtgggagga tatggtggct gagtttcatg atcaggtgcc ttttgatgga |
| 2761 | atgtggattg atatgaatga gccttctaat tttataagag gatctgagga tggatgtcct |
| 2821 | aataatgagc tagagaatcc tccttatgtg cctggagtag tgggaggaac actgcaggct |
| 2881 | gctacaattt gtgcttcttc tcatcagttt ctgtctacac attataatct gcataatctg |
| 2941 | tatggactga cagaggctat tgcttctcat agagctctgg tgaaggctag aggaacaaga |
| 3001 | ccttttgtga tttctagatc tacatttgct ggacatggaa gatatgctgg acattggaca |
| 3061 | ggagatgtat ggtcttcttg ggagcagcta gcttcttctg tgcctgagat tctgcagttt |
| 3121 | aatctgctag gagtgcctct ggtgggagct gatgtgtgtg gatttctggg aaatacatct |
| 3181 | gaggagctgt gtgtgagatg gacacagctg ggagcttttt atccttttat gagaaatcat |
| 3241 | aattctctgc tgtctctgcc tcaggagcct tattcttttt ctgagcctgc tcagcaggct |
| 3301 | atgagaaagg ctctgacact gagatatgct ctgctgcctc atctgtatac actgtttcat |
| 3361 | caggctcatg tggctggaga gacagtagct agacctctgt ttctggagtt tcctaaggat |
| 3421 | tcttctacat ggacagttga tcatcagctg ctgtggggag aggctctgct gattacacct |
| 3481 | gtgctgcagg ctggaaaggc tgaggtgaca ggatattttc ctctgggaac atggtatgat |
| 3541 | ctgcagacag tgcctataga ggctctggga tctctgcctc ctcctcctgc tgctcctaga |
| 3601 | gagcctgcta ttcattctga gggacagtgg gtgacactgc ctgctcctct ggatacaatt |
| 3661 | aatgtgcatc tgagagctgg atatattatt cctctgcagg gacctggact gacaacaaca |
| 3721 | gagtctagac agcagcctat ggctctggct gtggctctga caaagggagg agaggctaga |
| 3781 | ggagagctgt tttgggatga tggagagtct ctggaggtgc tggagagagg agcttataca |
| 3841 | caggtgattt ttctggctag aaataataca attgtgaatg agctggtgag agtgacatct |
| 3901 | gagggagctg gactgcagct gcagaaggtg acagtgctgg gagtggctac agctcctcag |
| 3961 | caggtgctgt ctaatggagt gcctgtgtct aattttacat attctcctga tacaaaggtg |
| 4021 | ctggatattt gtgtgtctct gctgatggga gagcagtttc tggtgtcttg gtgttaatga |
| 4081 | tttaaatctc gagccgggcg gagtgtgtta gtctctccag agggaggctg gttccccagg |
| 4141 | gaagcagagc ctgtgtgcgg gcagcagctg tgtgcgggcc tgggggttgt taagtgcaat |
| 4201 | tatttttaat aaaaggggca tttggaaaaa aaaaaaaaag gtagcagtcg acagatgaat |
| 4261 | tctgcagatc tgtggcttct agctgcccgg gtggcatccc tgtgacccct ccccagtgcc |
| 4321 | tctcctggcc ctggaagttg ccactccagt gcccaccagc cttgtcctaa taaaattaag |
| 4381 | ttgcatcatt ttgtctgact aggtgtcctt ctataatatt atggggtgga ggggggtggt |
| 4441 | atggagcaag gggcaagttg ggaagacaac ctgtagggcc tgcggggtct attgggaacc |
| 4501 | aagctggagt gcagtggcac aatcttggct cactgcaatc tccgcctcct gggttcaagc |
| 4561 | gattctcctg cctcagcctc ccgagttgtt gggattccag gcatgcatga ccaggctcag |
| 4621 | ctaatttttg tttttttggt agagacgggg tttcaccata ttggccaggc tggtctccaa |
| 4681 | ctcctaatct caggtgatct acccaccttg gcctcccaaa ttgctgggat tacaggcgtg |
| 4741 | aaccactgct cccttccctg tccttctgat tttaaaaaaa agagaaatgt tctggcacct |
| 4801 | gcacttgcac tggggacagc ctattttgct agtttgtttt gtttcgtttt gttttgatgg |
| 4861 | agagcgtatg ttgtttaaac gcggccgcgt agataagtag catggcgggt taatcattaa |
| 4921 | ctacaaggaa cccctagtga tggagttggc cactccctct ctgcgcgctc gctcgctcac |
| 4981 | tgaggccggg cgaccaaagg tcgcccgacg cccgggcttt gcccgggcgg cctcagtgag |
| 5041 | cgagcgagcg cgcagttaat taaggcgccc taggccgacc cttagactct gtactcagtt |
| 5101 | ctataaacga gccattggat acgagatccg tagattgata agggacacgg aatatccccg |
| 5161 | gacgcaatag acaccggtgg acagcttggt atcctgagca cagtcgcgcg tccgaatcta |
| 5221 | gctctacttt agaggccccg gattctgatg gtcgtagacc gcagaaccga ttggggggat |
| 5281 | gagatctact agttatcagc acacaattgc ccattatacg cgcgtataat ggactattgt |
| 5341 | gtgctgatat agggataaca gggtaattct agagctagca tatggatcca tcgatttgat |
| 5401 | gcggtatttt ctccttacgc atctgtgcgg tatttcacac cgcatacgtc aaagcaacca |
| 5461 | tagtacgcgc cctgtagcgg cgcattaagc gcggcgggtg tggtggttac gcgcagcgtg |
| 5521 | accgctacac ttgccagcgc cctagcgccc gctcctttcg ctttcttccc ttcctttctc |
| 5581 | gccacgttcg ccggctttcc ccgtcaagct ctaaatcggg ggctcccttt agggttccga |
| 5641 | tttagtgctt tacggcacct cgaccccaaa aaacttgatt tgggtgatgg ttcacgtagt |
| 5701 | gggccatcgc cctgatagac ggtttttcgc cctttgacgt tggagtccac gttctttaat |
| 5761 | agtggactct tgttccaaac tggaacaaca ctcaactcta tctcgggcta ttcttttgat |
| 5821 | ttataaggga ttttgccgat ttcggtctat tggttaaaaa atgagctgat ttaacaaaaa |
| 5881 | tttaacgcga attttaacaa aatattaacg tttacaattt tatggtgcac tctcagtaca |
| 5941 | atctgctctg atgccgcata gttaagccag ccccgacacc cgccaacacc cgctgacgcg |
| 6001 | ccctgacggg cttgtctgct cccggcatcc gcttacagac aagctgtgac cgtctccggg |
| 6061 | agctgcatgt gtcagaggtt ttcaccgtca tcaccgaaac gcgcgagacg aaagggcctc |
| 6121 | gtgatacgcc tatttttata ggttaatgtc atgataataa tggtttctta gacgtcaggt |
| 6181 | ggcacttttc ggggaaatgt gcgcggaacc cctatttgtt tatttttcta aatacattca |
| 6241 | aatatgtatc cgctcatgag acaataaccc tgataaatgc ttcaataata ttgaaaaagg |
| 6301 | aagagtatga gccatattca acgggaaacg tcgaggccgc gattaaattc caacatggat |
| 6361 | gctgatttat atgggtataa atgggctcgc gataatgtcg ggcaatcagg tgcgacaatc |
| 6421 | tatcgcttgt atgggaagcc cgatgcgcca gagttgtttc tgaaacatgg caaaggtagc |
| 6481 | gttgccaatg atgttacaga tgagatggtc agactaaact ggctgacgga atttatgcct |
| 6541 | cttccgacca tcaagcattt tatccgtact cctgatgatg catggttact caccactgcg |
| 6601 | atccccggaa aaacagcatt ccaggtatta gaagaatatc ctgattcagg tgaaaatatt |
| 6661 | gttgatgcgc tggcagtgtt cctgcgccgg ttgcattcga ttcctgtttg taattgtcct |
| 6721 | tttaacagcg atcgcgtatt tcgtctcgct caggcgcaat cacgaatgaa taacggtttg |
| 6781 | gttgatgcga gtgattttga tgacgagcgt aatggctggc ctgttgaaca agtctggaaa |
| 6841 | gaaatgcata aacttttgcc attctcaccg gattcagtcg tcactcatgg tgatttctca |
| 6901 | cttgataacc ttatttttga cgaggggaaa ttaataggtt gtattgatgt tggacgagtc |
| 6961 | ggaatcgcag accgatacca ggatcttgcc atcctatgga actgcctcgg tgagttttct |
| 7021 | ccttcattac agaaacggct ttttcaaaaa tatggtattg ataatcctga tatgaataaa |
| 7081 | ttgcagtttc atttgatgct cgatgagttt ttctaagcgt ataatggtct agagctagca |
| 7141 | tatggatcca tcgattccat tatacgcctg tcagaccaag tttactcata tatactttag |
| 7201 | attgatttaa aacttcattt ttaatttaaa aggatctagg tgaagatcct ttttgataat |
| 7261 | ctcatgacca aaatccctta acgtgagttt tcgttccact gagcgtcaga ccccgtagaa |
| 7321 | aagatcaaag gatcttcttg agatcctttt tttctgcgcg taatctgctg cttgcaaaca |
| 7381 | aaaaaaccac cgctaccagc ggtggtttgt ttgccggatc aagagctacc aactcttttt |
| 7441 | ccgaaggtaa ctggcttcag cagagcgcag ataccaaata ctgttcttct agtgtagccg |
| 7501 | tagttaggcc accacttcaa gaactctgta gcaccgccta catacctcgc tctgctaatc |
| 7561 | ctgttaccag tggctgctgc cagtggcgat aagtcgtgtc ttaccgggtt ggactcaaga |
| 7621 | cgatagttac cggataaggc gcagcggtcg ggctgaacgg ggggttcgtg cacacagccc |
| 7681 | agcttggagc gaacgaccta caccgaactg agatacctac agcgtgagct atgagaaagc |
| 7741 | gccacgcttc ccgaagggag aaaggcggac aggtatccgg taagcggcag ggtcggaaca |
| 7801 | ggagagcgca cgagggagct tccaggggga aacgcctggt atctttatag tcctgtcggg |
| 7861 | tttcgccacc tctgacttga gcgtcgattt ttgtgatgct cgtcaggggg gcggagccta |
| 7921 | tggaaaaacg ccagcaacgc ggccttttta cggttcctgg ccttttgctg gccttttgct |
| 7981 | cacatgt |

| pP071_seq100-IL2wt_25-56 deletion (SEQ ID NO: 476) | |
|---|---|
| (SEQ ID NO: 476) | |
| 1 | tctagagcta gcatatggat ccatcgattt agggataaca gggtaattat cagcacacaa |
| 61 | ttgcccatta tacgcgcgta taatggacta ttgtgtgctg atatctgtac acttaagggc |
| 121 | tagatcttag cttacgtcac tagagggtcc acgtttagtt tttaagatcc attgatctcc |
| 181 | taaacgctgc aagattcgca acctggtata cttagcctag gcgctaggtc ctagtgcagc |
| 241 | gggacttttt ttctaaagtc gttgagagga ggagtcgtca gaccagatag ctttgatgtc |
| 301 | ctgatcggaa ggatcgttgg cccccctgca ggcagctgtt aattaactgc gcgctcgctc |
| 361 | gctcactgag gccgcccggg caaagcccgg gcgtcgggcg acctttggtc gcccggcctc |
| 421 | agtgagcgag cgagcgcgca gagagggagt ggccaactcc atcactaggg gttccttgta |
| 481 | gttaatgatt aacccgccat gctacttatc tacggcgcgc cacgcgttct aggagttaat |
| 541 | ttttaaaaag cagtcaaaag tccaagtgcc cttgcgagca tttactctct ctgtttgctc |
| 601 | tggttaataa tctcaggagc acaaacattc cttactagtt ctaggagtta atttttaaaa |
| 661 | agcagtcaaa agtccaagtg cccttgcgag catttactct ctctgtttgc tctggttaat |
| 721 | aatctcagga gcacaaacat tccttactag ttctagagcg gccgccagtg tgctggaatt |
| 781 | cggctttttt agggctggaa gctacctttg acatcatttc ctctgcgaat gcatgtataa |
| 841 | tttctacaga acctattaga aaggatcacc cagcctctgc ttttgtacaa ctttccctta |
| 901 | aaaaactgcc aatcccactg ctgtttggcc caatagtgag aactttttcc tgctgcctct |
| 961 | tggtgctttt gcctatggcc cctattctgc ctgctgaaga cactcttgcc agcatggact |
| 1021 | taaacccctc cagctctgac aatcctcttt ctcttttgtt ttacatgaag ggtctggcag |
| 1081 | ccaaagcaat cactcaaagt tcaaacctta tcattttttg ctttgttcct cttggccttg |
| 1141 | gttttgtaca tcagctttga aaataccatc ccagggttaa tgctggggtt aatttataac |
| 1201 | tgagagtgct ctagttctgc aatacaggac atgctataaa aatggaaaga tgttgctttc |
| 1261 | tgagagatca ggtggattct tgggcatttg cttaagcttg gtaccttttt tatgggggtg |
| 1321 | aggcaccctc cctgttctca caggctgctg gctgtgtgtg ctctggtgtc tctggccact |
| 1381 | gccatgtaca gaatgcagct gctgtcctgc attgcactgt cactggcact ggttaccaac |
| 1441 | tcacagcagg gggccagcag gccaggcccc agggatgccc aggcccaccc tggcagaccc |
| 1501 | agggctgtgc ccacccagtg tgatgtgcct cccaacagca gatttgactg tgcccctgat |
| 1561 | aaggccatta ctcaggaaca gtgtgaggcc aggggctgct gctatatccc tgccaagcag |
| 1621 | ggcctgcagg gggcccagat ggggcagccc tggtgcttct tcccccccag ctacccctct |
| 1681 | tataagctgg agaatctgag cagctctgaa atgggctaca ctgccactct gaccaggact |
| 1741 | acccccacct tcttccccaa ggatattctg actctgaggc tggatgtgat gatggagact |
| 1801 | gagaacaggc tgcacttcac catcaaggac cctgccaaca ggaggtatga ggtgcccctg |
| 1861 | gaaactccca gggtgcactc tagggccccc agccccctgt attctgtgga gttctctgag |
| 1921 | gagccttttg gggtcattgt ccacaggcag ctggatggca gggtgctgct gaatactact |
| 1981 | gtggcccctc tgttctttgc tgaccagttc ctgcagctga gcaccagcct gccttctcag |
| 2041 | tacattactg gcctggctga gcatctgagc cccctgatgc tgagcacctc ttggaccaga |
| 2101 | atcaccctgt ggaacaggga cctggctccc actcctgggg ccaacctgta tggcagccac |
| 2161 | cccttctacc tggccctgga ggatgggggc tctgcccatg gggtgttcct gctgaacagc |
| 2221 | aatgccatgg atgtggtgct gcagccctct cctgccctgt cttggagatc tactgggggc |
| 2281 | atcctggatg tgtatatctt cctggggcct gagcccaagt ctgtggtgca gcagtacctg |
| 2341 | gatgtggtgg gctacccctt catgcccccc tactggggcc tgggcttcca cctgtgcagg |
| 2401 | tggggctact cttctactgc tatcaccagg caggtggtgg agaacatgac cagggctcac |
| 2461 | ttccctctgg atgtgcagtg gaatgacctg gactacatgg actctaggag agacttcact |
| 2521 | tttaataagg atgggttcag ggactttcct gccatggtgc aggagctgca tcaggggggc |
| 2581 | aggagatata tgatgattgt ggaccctgct atttctagct ctggccctgc tggcagctat |
| 2641 | aggccctatg atgaggggct gaggaggggg gtgttcatca ctaatgagac tggccagccc |
| 2701 | ctgattggca aggtgtggcc tggctctact gccttccctg atttcaccaa ccccactgcc |
| 2761 | ctggcctggt gggaggatat ggtggctgag tttcatgacc aggtgccctt tgatggcatg |
| 2821 | tggattgaca tgaatgagcc cagcaacttt atcaggggct ctgaagatgg ctgccccaac |
| 2881 | aatgagctgg agaacccccc ctatgtgcct ggggtggtgg ggggcaccct gcaggctgcc |
| 2941 | accatctgtg ccagcagcca ccagttcctg agcacccact ataacctgca caacctgtat |
| 3001 | ggcctgactg aggccattgc ctctcacagg gccctggtga aggctagggg gactaggccc |
| 3061 | tttgtgatca gcaggtctac ttttgctggc catggcaggt atgctgggca ctggactggg |
| 3121 | gatgtgtggt ctagctggga gcagctggcc agctctgtgc ctgagatcct gcagtttaat |
| 3181 | ctgctggggg tgcccctggt gggggctgat gtgtgtggct tcctgggcaa tacctctgag |
| 3241 | gagctgtgtg tgaggtggac tcagctgggg gctttctacc ccttcatgag aaaccacaac |
| 3301 | tctctgctga gcctgcccca ggagccctat tctttttctg agcctgccca gcaggctatg |
| 3361 | aggaaggccc tgactctgag gtatgccctg ctgccccacc tgtataccct gttccatcag |
| 3421 | gcccatgtgg ctggggagac tgtggccaga cctctgttcc tggagttccc caaggatagc |
| 3481 | tctacttgga ctgtggacca ccagctgctg tggggggagg ctctgctgat cacccctgtg |
| 3541 | ctgcaggctg ggaaggctga ggtgactggc tatttccccc tgggcacctg gtatgatctg |
| 3601 | cagactgtgc ccattgaggc cctgggctct ctgccccctc cccctgctgc ccccagggag |
| 3661 | cctgccatcc actctgaggg ccagtgggtg accctgcctg cccctctgga cactatcaat |
| 3721 | gtgcacctga gggctggcta catcatcccc ctgcagggcc ctggcctgac taccactgag |
| 3781 | tctaggcagc agcccatggc cctggctgtg gctctgacca agggggggga ggccaggggg |
| 3841 | gagctgttct gggatgatgg ggagtctctg gaggtgctgg agaggggggc ctacacccag |
| 3901 | gtgatcttcc tggctaggaa taacaccatt gtcaatgagc tggtgagggt gacctctgag |
| 3961 | ggggctggcc tgcagctgca gaaggtgact gtgctggggg tggctactgc cccccagcag |
| 4021 | gtgctgagca atggggtgcc tgtgagcaac ttcacctata gccctgacac caaggtgctg |
| 4081 | gacatctgtg tgagcctgct gatgggggag cagttcctgg tgagctggtg ctaatttaaa |
| 4141 | tctcgagccg ggcggagtgt gttagtctct ccagagggag gctggttccc cagggaagca |
| 4201 | gagcctgtgt gcgggcagca gctgtgtgcg ggcctggggg ttgttaagtg caattatttt |
| 4261 | taataaaagg ggcatttgga aaaaaaaaaa aaaggtagca gtcgacagat gaattctgca |
| 4321 | gatctgtggc ttctagctgc ccgggtggca tccctgtgac ccctccccag tgcctctcct |
| 4381 | ggccctggaa gttgccactc cagtgcccac cagccttgtc ctaataaaat taagttgcat |
| 4441 | cattttgtct gactaggtgt ccttctataa tattatgggg tggagggggg tggtatggag |
| 4501 | caaggggcaa gttgggaaga caacctgtag ggcctgcggg gtctattggg aaccaagctg |
| 4561 | gagtgcagtg gcacaatctt ggctcactgc aatctccgcc tcctgggttc aagcgattct |
| 4621 | cctgcctcag cctcccgagt tgttgggatt ccaggcatgc atgaccaggc tcagctaatt |
| 4681 | tttgtttttt tggtagagac ggggtttcac catattggcc aggctggtct ccaactccta |
| 4741 | atctcaggtg atctacccac cttggcctcc caaattgctg ggattacagg cgtgaaccac |
| 4801 | tgctcccttc cctgtccttc tgattttaaa aaaaagagaa atgttctggc acctgcactt |
| 4861 | gcactgggga cagcctattt tgctagtttg ttttgtttcg ttttgttttg atggagagcg |
| 4921 | tatgttgttt aaacgcggcc gcgtagataa gtagcatggc gggttaatca ttaactacaa |
| 4981 | ggaaccccta gtgatggagt tggccactcc ctctctgcgc gctcgctcgc tcactgaggc |
| 5041 | cgggcgacca aaggtcgccc gacgcccggg ctttgcccgg gcggcctcag tgagcgagcg |
| 5101 | agcgcgcagt taattaaggc gccctaggcc gacccttaga ctctgtactc agttctataa |
| 5161 | acgagccatt ggatacgaga tccgtagatt gataagggac acggaatatc cccggacgca |
| 5221 | atagacaccg gtggacagct tggtatcctg agcacagtcg cgcgtccgaa tctagctcta |
| 5281 | ctttagaggc cccggattct gatggtcgta gaccgcagaa ccgattgggg ggatgagatc |
| 5341 | tactagttat cagcacacaa ttgcccatta tacgcgcgta taatggacta ttgtgtgctg |
| 5401 | atatagggat aacagggtaa ttctagagct agcatatgga tccatcgatt tgatgcggta |
| 5461 | ttttctcctt acgcatctgt gcggtatttc acaccgcata cgtcaaagca accatagtac |
| 5521 | gcgccctgta gcggcgcatt aagcgcggcg ggtgtggtgg ttacgcgcag cgtgaccgct |
| 5581 | acacttgcca gcgccctagc gcccgctcct ttcgctttct tcccttcctt tctcgccacg |
| 5641 | ttcgccggct ttccccgtca agctctaaat cgggggctcc ctttagggtt ccgatttagt |
| 5701 | gctttacggc acctcgaccc caaaaaactt gatttgggtg atggttcacg tagtgggcca |
| 5761 | tcgccctgat agacggtttt tcgccctttg acgttggagt ccacgttctt taatagtgga |
| 5821 | ctcttgttcc aaactggaac aacactcaac tctatctcgg gctattcttt tgatttataa |
| 5881 | gggattttgc cgatttcggt ctattggtta aaaaatgagc tgatttaaca aaaatttaac |
| 5941 | gcgaatttta acaaaatatt aacgtttaca attttatggt gcactctcag tacaatctgc |
| 6001 | tctgatgccg catagttaag ccagccccga cacccgccaa cacccgctga cgcgccctga |
| 6061 | cgggcttgtc tgctcccggc atccgcttac agacaagctg tgaccgtctc cgggagctgc |
| 6121 | atgtgtcaga ggttttcacc gtcatcaccg aaacgcgcga gacgaaaggg cctcgtgata |
| 6181 | cgcctatttt tataggttaa tgtcatgata ataatggttt cttagacgtc aggtggcact |
| 6241 | tttcggggaa atgtgcgcgg aacccctatt tgtttatttt tctaaataca ttcaaatatg |
| 6301 | tatccgctca tgagacaata accctgataa atgcttcaat aatattgaaa aaggaagagt |
| 6361 | atgagccata ttcaacggga aacgtcgagg ccgcgattaa attccaacat ggatgctgat |
| 6421 | ttatatgggt ataaatgggc tcgcgataat gtcgggcaat caggtgcgac aatctatcgc |
| 6481 | ttgtatggga agcccgatgc gccagagttg tttctgaaac atggcaaagg tagcgttgcc |
| 6541 | aatgatgtta cagatgagat ggtcagacta aactggctga cggaatttat gcctcttccg |
| 6601 | accatcaagc attttatccg tactcctgat gatgcatggt tactcaccac tgcgatcccc |
| 6661 | ggaaaaacag cattccaggt attagaagaa tatcctgatt caggtgaaaa tattgttgat |
| 6721 | gcgctggcag tgttcctgcg ccggttgcat tcgattcctg tttgtaattg tccttttaac |
| 6781 | agcgatcgcg tatttcgtct cgctcaggcg caatcacgaa tgaataacgg tttggttgat |
| 6841 | gcgagtgatt ttgatgacga gcgtaatggc tggcctgttg aacaagtctg gaaagaaatg |
| 6901 | cataaacttt tgccattctc accggattca gtcgtcactc atggtgattt ctcacttgat |
| 6961 | aaccttattt ttgacgaggg gaaattaata ggttgtattg atgttggacg agtcggaatc |
| 7021 | gcagaccgat accaggatct tgccatccta tggaactgcc tcggtgagtt ttctccttca |
| 7081 | ttacagaaac ggctttttca aaaatatggt attgataatc ctgatatgaa taaattgcag |
| 7141 | tttcatttga tgctcgatga gtttttctaa gcgtataatg gtctagagct agcatatgga |
| 7201 | tccatcgatt ccattatacg cctgtcagac caagtttact catatatact ttagattgat |
| 7261 | ttaaaacttc atttttaatt taaaaggatc taggtgaaga tcctttttga taatctcatg |
| 7321 | accaaaatcc cttaacgtga gttttcgttc cactgagcgt cagaccccgt agaaaagatc |
| 7381 | aaaggatctt cttgagatcc tttttttctg cgcgtaatct gctgcttgca aacaaaaaaa |
| 7441 | ccaccgctac cagcggtggt ttgtttgccg gatcaagagc taccaactct ttttccgaag |
| 7501 | gtaactggct tcagcagagc gcagatacca aatactgttc ttctagtgta gccgtagtta |
| 7561 | ggccaccact tcaagaactc tgtagcaccg cctacatacc tcgctctgct aatcctgtta |
| 7621 | ccagtggctg ctgccagtgg cgataagtcg tgtcttaccg ggttggactc aagacgatag |
| 7681 | ttaccggata aggcgcagcg gtcgggctga acggggggtt cgtgcacaca gcccagcttg |
| 7741 | gagcgaacga cctacaccga actgagatac ctacagcgtg agctatgaga aagcgccacg |
| 7801 | cttcccgaag ggagaaaggc ggacaggtat ccggtaagcg gcagggtcgg aacaggagag |
| 7861 | cgcacgaggg agcttccagg gggaaacgcc tggtatcttt atagtcctgt cgggtttcgc |
| 7921 | cacctctgac ttgagcgtcg atttttgtga tgctcgtcag gggggcggag cctatggaaa |
| 7981 | aacgccagca acgcggcctt tttacggttc ctggcctttt gctggccttt tgctcacatg |
| 8041 | t |

| pP072_seq100-201Ig_25-56 deletion (SEQ ID NO: 477) | |
|---|---|
| (SEQ ID NO: 477) | |
| 1 | tctagagcta gcatatggat ccatcgattt agggataaca gggtaattat cagcacacaa |
| 61 | ttgcccatta tacgcgcgta taatggacta ttgtgtgctg atatctgtac acttaagggc |
| 121 | tagatcttag cttacgtcac tagagggtcc acgtttagtt tttaagatcc attgatctcc |
| 181 | taaacgctgc aagattcgca acctggtata cttagcctag gcgctaggtc ctagtgcagc |
| 241 | gggacttttt ttctaaagtc gttgagagga ggagtcgtca gaccagatag ctttgatgtc |
| 301 | ctgatcggaa ggatcgttgg cccccctgca ggcagctgtt aattaactgc gcgctcgctc |
| 361 | gctcactgag gccgcccggg caaagcccgg gcgtcgggcg acctttggtc gcccggcctc |
| 421 | agtgagcgag cgagcgcgca gagagggagt ggccaactcc atcactaggg gttccttgta |
| 481 | gttaatgatt aacccgccat gctacttatc tacggcgcgc cacgcgttct aggagttaat |
| 541 | ttttaaaaag cagtcaaaag tccaagtgcc cttgcgagca tttactctct ctgtttgctc |
| 601 | tggttaataa tctcaggagc acaaacattc cttactagtt ctaggagtta atttttaaaa |
| 661 | agcagtcaaa agtccaagtg cccttgcgag catttactct ctctgtttgc tctggttaat |
| 721 | aatctcagga gcacaaacat tccttactag ttctagagcg gccgccagtg tgctggaatt |
| 781 | cggctttttt agggctggaa gctacctttg acatcatttc ctctgcgaat gcatgtataa |
| 841 | tttctacaga acctattaga aaggatcacc cagcctctgc ttttgtacaa ctttccctta |
| 901 | aaaaactgcc aatcccactg ctgtttggcc caatagtgag aactttttcc tgctgcctct |
| 961 | tggtgctttt gcctatggcc cctattctgc ctgctgaaga cactcttgcc agcatggact |
| 1021 | taaacccctc cagctctgac aatcctcttt ctcttttgtt ttacatgaag ggtctggcag |
| 1081 | ccaaagcaat cactcaaagt tcaaacctta tcattttttg ctttgttcct cttggccttg |
| 1141 | gttttgtaca tcagctttga aaataccatc ccagggttaa tgctggggtt aatttataac |
| 1201 | tgagagtgct ctagttctgc aatacaggac atgctataaa aatggaaaga tgttgctttc |
| 1261 | tgagagatca ggtggattct tgggcatttg cttaagcttg gtaccttttt tatgggggtg |
| 1321 | aggcaccctc cctgttctca caggctgctg gctgtgtgtg ctctggtgtc tctggccact |
| 1381 | gccatggagt ttgggctgag ctgggtcttt ctggtggccc tgctgaaggg agtccagtgt |
| 1441 | gagcagcagg gggccagcag gccaggcccc agggatgccc aggcccaccc tggcagaccc |
| 1501 | agggctgtgc ccacccagtg tgatgtgcct cccaacagca gatttgactg tgcccctgat |
| 1561 | aaggccatta ctcaggaaca gtgtgaggcc aggggctgct gctatatccc tgccaagcag |
| 1621 | ggcctgcagg gggcccagat ggggcagccc tggtgcttct tcccccccag ctacccctct |
| 1681 | tataagctgg agaatctgag cagctctgaa atgggctaca ctgccactct gaccaggact |
| 1741 | acccccacct tcttccccaa ggatattctg actctgaggc tggatgtgat gatggagact |
| 1801 | gagaacaggc tgcacttcac catcaaggac cctgccaaca ggaggtatga ggtgcccctg |
| 1861 | gaaactccca gggtgcactc tagggccccc agccccctgt attctgtgga gttctctgag |
| 1921 | gagccttttg gggtcattgt ccacaggcag ctggatggca gggtgctgct gaatactact |
| 1981 | gtggcccctc tgttctttgc tgaccagttc ctgcagctga gcaccagcct gccttctcag |
| 2041 | tacattactg gcctggctga gcatctgagc cccctgatgc tgagcacctc ttggaccaga |
| 2101 | atcaccctgt ggaacaggga cctggctccc actcctgggg ccaacctgta tggcagccac |
| 2161 | cccttctacc tggccctgga ggatgggggc tctgcccatg gggtgttcct gctgaacagc |
| 2221 | aatgccatgg atgtggtgct gcagccctct cctgccctgt cttggagatc tactgggggc |
| 2281 | atcctggatg tgtatatctt cctggggcct gagcccaagt ctgtggtgca gcagtacctg |
| 2341 | gatgtggtgg gctacccctt catgcccccc tactggggcc tgggcttcca cctgtgcagg |
| 2401 | tggggctact cttctactgc tatcaccagg caggtggtgg agaacatgac cagggctcac |
| 2461 | ttccctctgg atgtgcagtg gaatgacctg gactacatgg actctaggag agacttcact |
| 2521 | tttaataagg atgggttcag ggactttcct gccatggtgc aggagctgca tcaggggggc |
| 2581 | aggagatata tgatgattgt ggaccctgct atttctagct ctggccctgc tggcagctat |
| 2641 | aggccctatg atgaggggct gaggaggggg gtgttcatca ctaatgagac tggccagccc |
| 2701 | ctgattggca aggtgtggcc tggctctact gccttccctg atttcaccaa ccccactgcc |
| 2761 | ctggcctggt gggaggatat ggtggctgag tttcatgacc aggtgccctt tgatggcatg |
| 2821 | tggattgaca tgaatgagcc cagcaacttt atcaggggct ctgaagatgg ctgccccaac |
| 2881 | aatgagctgg agaacccccc ctatgtgcct ggggtggtgg ggggcaccct gcaggctgcc |
| 2941 | accatctgtg ccagcagcca ccagttcctg agcacccact ataacctgca caacctgtat |
| 3001 | ggcctgactg aggccattgc ctctcacagg gccctggtga aggctagggg gactaggccc |
| 3061 | tttgtgatca gcaggtctac ttttgctggc catggcaggt atgctgggca ctggactggg |
| 3121 | gatgtgtggt ctagctggga gcagctggcc agctctgtgc ctgagatcct gcagtttaat |
| 3181 | ctgctggggg tgcccctggt gggggctgat gtgtgtggct tcctgggcaa tacctctgag |
| 3241 | gagctgtgtg tgaggtggac tcagctgggg gctttctacc ccttcatgag aaaccacaac |
| 3301 | tctctgctga gcctgcccca ggagccctat tctttttctg agcctgccca gcaggctatg |
| 3361 | aggaaggccc tgactctgag gtatgccctg ctgccccacc tgtataccct gttccatcag |
| 3421 | gcccatgtgg ctggggagac tgtggccaga cctctgttcc tggagttccc caaggatagc |
| 3481 | tctacttgga ctgtggacca ccagctgctg tggggggagg ctctgctgat cacccctgtg |
| 3541 | ctgcaggctg ggaaggctga ggtgactggc tatttccccc tgggcacctg gtatgatctg |
| 3601 | cagactgtgc ccattgaggc cctgggctct ctgccccctc cccctgctgc ccccagggag |
| 3661 | cctgccatcc actctgaggg ccagtgggtg accctgcctg cccctctgga cactatcaat |
| 3721 | gtgcacctga gggctggcta catcatcccc ctgcagggcc ctggcctgac taccactgag |
| 3781 | tctaggcagc agcccatggc cctggctgtg gctctgacca agggggggga ggccaggggg |
| 3841 | gagctgttct gggatgatgg ggagtctctg gaggtgctgg agaggggggc ctacacccag |
| 3901 | gtgatcttcc tggctaggaa taacaccatt gtcaatgagc tggtgagggt gacctctgag |
| 3961 | ggggctggcc tgcagctgca gaaggtgact gtgctggggg tggctactgc cccccagcag |
| 4021 | gtgctgagca atggggtgcc tgtgagcaac ttcacctata gccctgacac caaggtgctg |
| 4081 | gacatctgtg tgagcctgct gatgggggag cagttcctgg tgagctggtg ctaatttaaa |
| 4141 | tctcgagccg ggcggagtgt gttagtctct ccagagggag gctggttccc cagggaagca |
| 4201 | gagcctgtgt gcgggcagca gctgtgtgcg ggcctggggg ttgttaagtg caattatttt |
| 4261 | taataaaagg ggcatttgga aaaaaaaaaa aaaggtagca gtcgacagat gaattctgca |
| 4321 | gatctgtggc ttctagctgc ccgggtggca tccctgtgac ccctccccag tgcctctcct |
| 4381 | ggccctggaa gttgccactc cagtgcccac cagccttgtc ctaataaaat taagttgcat |
| 4441 | cattttgtct gactaggtgt ccttctataa tattatgggg tggagggggg tggtatggag |
| 4501 | caaggggcaa gttgggaaga caacctgtag ggcctgcggg gtctattggg aaccaagctg |
| 4561 | gagtgcagtg gcacaatctt ggctcactgc aatctccgcc tcctgggttc aagcgattct |
| 4621 | cctgcctcag cctcccgagt tgttgggatt ccaggcatgc atgaccaggc tcagctaatt |
| 4681 | tttgtttttt tggtagagac ggggtttcac catattggcc aggctggtct ccaactccta |
| 4741 | atctcaggtg atctacccac cttggcctcc caaattgctg ggattacagg cgtgaaccac |
| 4801 | tgctcccttc cctgtccttc tgattttaaa aaaaagagaa atgttctggc acctgcactt |
| 4861 | gcactgggga cagcctattt tgctagtttg ttttgtttcg ttttgttttg atggagagcg |
| 4921 | tatgttgttt aaacgcggcc gcgtagataa gtagcatggc gggttaatca ttaactacaa |
| 4981 | ggaaccccta gtgatggagt tggccactcc ctctctgcgc gctcgctcgc tcactgaggc |
| 5041 | cgggcgacca aaggtcgccc gacgcccggg ctttgcccgg gcggcctcag tgagcgagcg |
| 5101 | agcgcgcagt taattaaggc gccctaggcc gacccttaga ctctgtactc agttctataa |
| 5161 | acgagccatt ggatacgaga tccgtagatt gataagggac acggaatatc cccggacgca |
| 5221 | atagacaccg gtggacagct tggtatcctg agcacagtcg cgcgtccgaa tctagctcta |
| 5281 | ctttagaggc cccggattct gatggtcgta gaccgcagaa ccgattgggg ggatgagatc |
| 5341 | tactagttat cagcacacaa ttgcccatta tacgcgcgta taatggacta ttgtgtgctg |
| 5401 | atatagggat aacagggtaa ttctagagct agcatatgga tccatcgatt tgatgcggta |
| 5461 | ttttctcctt acgcatctgt gcggtatttc acaccgcata cgtcaaagca accatagtac |
| 5521 | gcgccctgta gcggcgcatt aagcgcggcg ggtgtggtgg ttacgcgcag cgtgaccgct |
| 5581 | acacttgcca gcgccctagc gcccgctcct ttcgctttct tcccttcctt tctcgccacg |
| 5641 | ttcgccggct ttccccgtca agctctaaat cgggggctcc ctttagggtt ccgatttagt |
| 5701 | gctttacggc acctcgaccc caaaaaactt gatttgggtg atggttcacg tagtgggcca |
| 5761 | tcgccctgat agacggtttt tcgccctttg acgttggagt ccacgttctt taatagtgga |
| 5821 | ctcttgttcc aaactggaac aacactcaac tctatctcgg gctattcttt tgatttataa |
| 5881 | gggattttgc cgatttcggt ctattggtta aaaaatgagc tgatttaaca aaaatttaac |
| 5941 | gcgaatttta acaaaatatt aacgtttaca attttatggt gcactctcag tacaatctgc |
| 6001 | tctgatgccg catagttaag ccagccccga cacccgccaa cacccgctga cgcgccctga |
| 6061 | cgggcttgtc tgctcccggc atccgcttac agacaagctg tgaccgtctc cgggagctgc |
| 6121 | atgtgtcaga ggttttcacc gtcatcaccg aaacgcgcga gacgaaaggg cctcgtgata |
| 6181 | cgcctatttt tataggttaa tgtcatgata ataatggttt cttagacgtc aggtggcact |
| 6241 | tttcggggaa atgtgcgcgg aacccctatt tgtttatttt tctaaataca ttcaaatatg |
| 6301 | tatccgctca tgagacaata accctgataa atgcttcaat aatattgaaa aaggaagagt |
| 6361 | atgagccata ttcaacggga aacgtcgagg ccgcgattaa attccaacat ggatgctgat |
| 6421 | ttatatgggt ataaatgggc tcgcgataat gtcgggcaat caggtgcgac aatctatcgc |
| 6481 | ttgtatggga agcccgatgc gccagagttg tttctgaaac atggcaaagg tagcgttgcc |
| 6541 | aatgatgtta cagatgagat ggtcagacta aactggctga cggaatttat gcctcttccg |
| 6601 | accatcaagc attttatccg tactcctgat gatgcatggt tactcaccac tgcgatcccc |
| 6661 | ggaaaaacag cattccaggt attagaagaa tatcctgatt caggtgaaaa tattgttgat |
| 6721 | gcgctggcag tgttcctgcg ccggttgcat tcgattcctg tttgtaattg tccttttaac |
| 6781 | agcgatcgcg tatttcgtct cgctcaggcg caatcacgaa tgaataacgg tttggttgat |
| 6841 | gcgagtgatt ttgatgacga gcgtaatggc tggcctgttg aacaagtctg gaaagaaatg |
| 6901 | cataaacttt tgccattctc accggattca gtcgtcactc atggtgattt ctcacttgat |
| 6961 | aaccttattt ttgacgaggg gaaattaata ggttgtattg atgttggacg agtcggaatc |
| 7021 | gcagaccgat accaggatct tgccatccta tggaactgcc tcggtgagtt ttctccttca |
| 7081 | ttacagaaac ggctttttca aaaatatggt attgataatc ctgatatgaa taaattgcag |
| 7141 | tttcatttga tgctcgatga gtttttctaa gcgtataatg gtctagagct agcatatgga |
| 7201 | tccatcgatt ccattatacg cctgtcagac caagtttact catatatact ttagattgat |
| 7261 | ttaaaacttc atttttaatt taaaaggatc taggtgaaga tcctttttga taatctcatg |
| 7321 | accaaaatcc cttaacgtga gttttcgttc cactgagcgt cagaccccgt agaaaagatc |
| 7381 | aaaggatctt cttgagatcc tttttttctg cgcgtaatct gctgcttgca aacaaaaaaa |
| 7441 | ccaccgctac cagcggtggt ttgtttgccg gatcaagagc taccaactct ttttccgaag |
| 7501 | gtaactggct tcagcagagc gcagatacca aatactgttc ttctagtgta gccgtagtta |
| 7561 | ggccaccact tcaagaactc tgtagcaccg cctacatacc tcgctctgct aatcctgtta |
| 7621 | ccagtggctg ctgccagtgg cgataagtcg tgtcttaccg ggttggactc aagacgatag |
| 7681 | ttaccggata aggcgcagcg gtcgggctga acggggggtt cgtgcacaca gcccagcttg |
| 7741 | gagcgaacga cctacaccga actgagatac ctacagcgtg agctatgaga aagcgccacg |
| 7801 | cttcccgaag ggagaaaggc ggacaggtat ccggtaagcg gcagggtcgg aacaggagag |
| 7861 | cgcacgaggg agcttccagg gggaaacgcc tggtatcttt atagtcctgt cgggtttcgc |
| 7921 | cacctctgac ttgagcgtcg atttttgtga tgctcgtcag gggggcggag cctatggaaa |
| 7981 | aacgccagca acgcggcctt tttacggttc ctggcctttt gctggccttt tgctcacatg |
| 8041 | t |

| pP073_seq3-IL2wt 25-56 deletion (SEQ ID NO: 478) | |
|---|---|
| (SEQ ID NO: 478) | |
| 1 | tctagagcta gcatatggat ccatcgattt agggataaca gggtaattat cagcacacaa |
| 61 | ttgcccatta tacgcgcgta taatggacta ttgtgtgctg atatctgtac acttaagggc |
| 121 | tagatcttag cttacgtcac tagagggtcc acgtttagtt tttaagatcc attgatctcc |
| 181 | taaacgctgc aagattcgca acctggtata cttagcctag gcgctaggtc ctagtgcagc |
| 241 | gggacttttt ttctaaagtc gttgagagga ggagtcgtca gaccagatag ctttgatgtc |
| 301 | ctgatcggaa ggatcgttgg cccccctgca ggcagctgtt aattaactgc gcgctcgctc |
| 361 | gctcactgag gccgcccggg caaagcccgg gcgtcgggcg acctttggtc gcccggcctc |
| 421 | agtgagcgag cgagcgcgca gagagggagt ggccaactcc atcactaggg gttccttgta |
| 481 | gttaatgatt aacccgccat gctacttatc tacggcgcgc cacgcgttct aggagttaat |
| 541 | ttttaaaaag cagtcaaaag tccaagtgcc cttgcgagca tttactctct ctgtttgctc |
| 601 | tggttaataa tctcaggagc acaaacattc cttactagtt ctaggagtta atttttaaaa |
| 661 | agcagtcaaa agtccaagtg cccttgcgag catttactct ctctgtttgc tctggttaat |
| 721 | aatctcagga gcacaaacat tccttactag ttctagagcg gccgccagtg tgctggaatt |
| 781 | cggctttttt agggctggaa gctacctttg acatcatttc ctctgcgaat gcatgtataa |
| 841 | tttctacaga acctattaga aaggatcacc cagcctctgc ttttgtacaa ctttccctta |
| 901 | aaaaactgcc aatcccactg ctgtttggcc caatagtgag aactttttcc tgctgcctct |
| 961 | tggtgctttt gcctatggcc cctattctgc ctgctgaaga cactcttgcc agcatggact |
| 1021 | taaacccctc cagctctgac aatcctcttt ctcttttgtt ttacatgaag ggtctggcag |
| 1081 | ccaaagcaat cactcaaagt tcaaacctta tcattttttg ctttgttcct cttggccttg |
| 1141 | gttttgtaca tcagctttga aaataccatc ccagggttaa tgctggggtt aatttataac |
| 1201 | tgagagtgct ctagttctgc aatacaggac atgctataaa aatggaaaga tgttgctttc |
| 1261 | tgagagatca ggtggattct tgggcatttg cttaagcttg gtaccttttt tatgggagtg |
| 1321 | agacatcctc cttgttctca tagactgctg gctgtgtgtg ctctggtgtc tctggctaca |
| 1381 | gccatgtaca gaatgcagct gctgtcctgc attgcactgt cactggcact ggttaccaac |
| 1441 | tcacagcagg gagcttctag acctggacct agagatgctc aggctcatcc tggaagacct |
| 1501 | agagctgtgc ctacacagtg tgatgtgcct cctaattcta gatttgattg tgctcctgat |
| 1561 | aaggctataa cacaggagca gtgtgaggct agaggatgtt gttatattcc tgctaagcag |
| 1621 | ggactgcagg gagctcagat gggacagcct tggtgttttt ttcctccttc ttatccttct |
| 1681 | tataagctgg agaatctgtc ttcttctgag atgggatata cagctacact gacaagaaca |
| 1741 | acacctacat tttttcctaa ggatattctg acactgagac tggatgtgat gatggagaca |
| 1801 | gagaatagac tgcattttac aattaaggat cctgctaata gaagatatga ggtgcctcta |
| 1861 | gagacaccta gagtgcattc tagagctcct tctcctctgt attctgtgga gttttctgag |
| 1921 | gagccttttg gagtgattgt gcatagacag ctggatggaa gagtgctgct gaatacaaca |
| 1981 | gtggctcctc tgttttttgc tgatcagttt ctgcagctgt ctacatctct gccttctcag |
| 2041 | tatattacag gactggctga gcatctgtct cctctgatgc tgtctacatc ttggacaaga |
| 2101 | attacactgt ggaatagaga tctggctcct acacctggag ctaatctgta tggatctcat |
| 2161 | cctttttatc tggctctgga ggatggagga tctgctcatg gagtgtttct gctgaattct |
| 2221 | aatgctatgg atgtagtgct gcagccttct cctgctctgt cttggagatc tacaggagga |
| 2281 | attctggatg tgtatatttt tctaggacct gagcctaagt ctgtggtgca gcagtatctg |
| 2341 | gatgtagtgg gatatccttt tatgcctcct tattggggac tgggatttca tctgtgtaga |
| 2401 | tggggatatt cttctacagc tattacaaga caggtggttg agaatatgac aagagctcat |
| 2461 | tttcctctgg atgtgcagtg gaatgatcta gattatatgg attctagaag agattttaca |
| 2521 | tttaataagg atggatttag agattttcct gctatggtgc aggagctgca tcagggagga |
| 2581 | agaagatata tgatgattgt ggatcctgct atttcttctt ctggacctgc tggatcttat |
| 2641 | agaccttatg atgagggact gagaagagga gtgtttatta caaatgagac aggacagcct |
| 2701 | ctgattggaa aggtgtggcc tggatctaca gcttttcctg attttacaaa tcctacagct |
| 2761 | ctggcttggt gggaggatat ggtggctgag tttcatgatc aggtgccttt tgatggaatg |
| 2821 | tggattgata tgaatgagcc ttctaatttt ataagaggat ctgaggatgg atgtcctaat |
| 2881 | aatgagctag agaatcctcc ttatgtgcct ggagtagtgg gaggaacact gcaggctgct |
| 2941 | acaatttgtg cttcttctca tcagtttctg tctacacatt ataatctgca taatctgtat |
| 3001 | ggactgacag aggctattgc ttctcataga gctctggtga aggctagagg aacaagacct |
| 3061 | tttgtgattt ctagatctac atttgctgga catggaagat atgctggaca ttggacagga |
| 3121 | gatgtatggt cttcttggga gcagctagct tcttctgtgc ctgagattct gcagtttaat |
| 3181 | ctgctaggag tgcctctggt gggagctgat gtgtgtggat ttctgggaaa tacatctgag |
| 3241 | gagctgtgtg tgagatggac acagctggga gctttttatc cttttatgag aaatcataat |
| 3301 | tctctgctgt ctctgcctca ggagccttat tctttttctg agcctgctca gcaggctatg |
| 3361 | agaaaggctc tgacactgag atatgctctg ctgcctcatc tgtatacact gtttcatcag |
| 3421 | gctcatgtgg ctggagagac agtagctaga cctctgtttc tggagtttcc taaggattct |
| 3481 | tctacatgga cagttgatca tcagctgctg tggggagagg ctctgctgat tacacctgtg |
| 3541 | ctgcaggctg gaaaggctga ggtgacagga tattttcctc tgggaacatg gtatgatctg |
| 3601 | cagacagtgc ctatagaggc tctgggatct ctgcctcctc ctcctgctgc tcctagagag |
| 3661 | cctgctattc attctgaggg acagtgggtg acactgcctg ctcctctgga tacaattaat |
| 3721 | gtgcatctga gagctggata tattattcct ctgcagggac ctggactgac aacaacagag |
| 3781 | tctagacagc agcctatggc tctggctgtg gctctgacaa agggaggaga ggctagagga |
| 3841 | gagctgtttt gggatgatgg agagtctctg gaggtgctgg agagaggagc ttatacacag |
| 3901 | gtgatttttc tggctagaaa taatacaatt gtgaatgagc tggtgagagt gacatctgag |
| 3961 | ggagctggac tgcagctgca gaaggtgaca gtgctgggag tggctacagc tcctcagcag |
| 4021 | gtgctgtcta atggagtgcc tgtgtctaat tttacatatt ctcctgatac aaaggtgctg |
| 4081 | gatatttgtg tgtctctgct gatgggagag cagtttctgg tgtcttggtg ttaatgattt |
| 4141 | aaatctcgag ccgggcggag tgtgttagtc tctccagagg gaggctggtt ccccagggaa |
| 4201 | gcagagcctg tgtgcgggca gcagctgtgt gcgggcctgg gggttgttaa gtgcaattat |
| 4261 | ttttaataaa aggggcattt ggaaaaaaaa aaaaaaggta gcagtcgaca gatgaattct |
| 4321 | gcagatctgt ggcttctagc tgcccgggtg gcatccctgt gacccctccc cagtgcctct |
| 4381 | cctggccctg gaagttgcca ctccagtgcc caccagcctt gtcctaataa aattaagttg |
| 4441 | catcattttg tctgactagg tgtccttcta taatattatg gggtggaggg gggtggtatg |
| 4501 | gagcaagggg caagttggga agacaacctg tagggcctgc ggggtctatt gggaaccaag |
| 4561 | ctggagtgca gtggcacaat cttggctcac tgcaatctcc gcctcctggg ttcaagcgat |
| 4621 | tctcctgcct cagcctcccg agttgttggg attccaggca tgcatgacca ggctcagcta |
| 4681 | atttttgttt ttttggtaga gacggggttt caccatattg gccaggctgg tctccaactc |
| 4741 | ctaatctcag gtgatctacc caccttggcc tcccaaattg ctgggattac aggcgtgaac |
| 4801 | cactgctccc ttccctgtcc ttctgatttt aaaaaaaaga gaaatgttct ggcacctgca |
| 4861 | cttgcactgg ggacagccta ttttgctagt ttgttttgtt tcgttttgtt ttgatggaga |
| 4921 | gcgtatgttg tttaaacgcg gccgcgtaga taagtagcat ggcgggttaa tcattaacta |
| 4981 | caaggaaccc ctagtgatgg agttggccac tccctctctg cgcgctcgct cgctcactga |
| 5041 | ggccgggcga ccaaaggtcg cccgacgccc gggctttgcc cgggcggcct cagtgagcga |
| 5101 | gcgagcgcgc agttaattaa ggcgccctag gccgaccctt agactctgta ctcagttcta |
| 5161 | taaacgagcc attggatacg agatccgtag attgataagg gacacggaat atccccggac |
| 5221 | gcaatagaca ccggtggaca gcttggtatc ctgagcacag tcgcgcgtcc gaatctagct |
| 5281 | ctactttaga ggccccggat tctgatggtc gtagaccgca gaaccgattg gggggatgag |
| 5341 | atctactagt tatcagcaca caattgccca ttatacgcgc gtataatgga ctattgtgtg |
| 5401 | ctgatatagg gataacaggg taattctaga gctagcatat ggatccatcg atttgatgcg |
| 5461 | gtattttctc cttacgcatc tgtgcggtat ttcacaccgc atacgtcaaa gcaaccatag |
| 5521 | tacgcgccct gtagcggcgc attaagcgcg gcgggtgtgg tggttacgcg cagcgtgacc |
| 5581 | gctacacttg ccagcgccct agcgcccgct cctttcgctt tcttcccttc ctttctcgcc |
| 5641 | acgttcgccg gctttccccg tcaagctcta aatcgggggc tccctttagg gttccgattt |
| 5701 | agtgctttac ggcacctcga ccccaaaaaa cttgatttgg gtgatggttc acgtagtggg |
| 5761 | ccatcgccct gatagacggt ttttcgccct ttgacgttgg agtccacgtt ctttaatagt |
| 5821 | ggactcttgt tccaaactgg aacaacactc aactctatct cgggctattc ttttgattta |
| 5881 | taagggattt tgccgatttc ggtctattgg ttaaaaaatg agctgattta acaaaaattt |
| 5941 | aacgcgaatt ttaacaaaat attaacgttt acaattttat ggtgcactct cagtacaatc |
| 6001 | tgctctgatg ccgcatagtt aagccagccc cgacacccgc caacacccgc tgacgcgccc |
| 6061 | tgacgggctt gtctgctccc ggcatccgct tacagacaag ctgtgaccgt ctccgggagc |
| 6121 | tgcatgtgtc agaggttttc accgtcatca ccgaaacgcg cgagacgaaa gggcctcgtg |
| 6181 | atacgcctat ttttataggt taatgtcatg ataataatgg tttcttagac gtcaggtggc |
| 6241 | acttttcggg gaaatgtgcg cggaacccct atttgtttat ttttctaaat acattcaaat |
| 6301 | atgtatccgc tcatgagaca ataaccctga taaatgcttc aataatattg aaaaaggaag |
| 6361 | agtatgagcc atattcaacg ggaaacgtcg aggccgcgat taaattccaa catggatgct |
| 6421 | gatttatatg ggtataaatg ggctcgcgat aatgtcgggc aatcaggtgc gacaatctat |
| 6481 | cgcttgtatg ggaagcccga tgcgccagag ttgtttctga aacatggcaa aggtagcgtt |
| 6541 | gccaatgatg ttacagatga gatggtcaga ctaaactggc tgacggaatt tatgcctctt |
| 6601 | ccgaccatca agcattttat ccgtactcct gatgatgcat ggttactcac cactgcgatc |
| 6661 | cccggaaaaa cagcattcca ggtattagaa gaatatcctg attcaggtga aaatattgtt |
| 6721 | gatgcgctgg cagtgttcct gcgccggttg cattcgattc ctgtttgtaa ttgtcctttt |
| 6781 | aacagcgatc gcgtatttcg tctcgctcag gcgcaatcac gaatgaataa cggtttggtt |
| 6841 | gatgcgagtg attttgatga cgagcgtaat ggctggcctg ttgaacaagt ctggaaagaa |
| 6901 | atgcataaac ttttgccatt ctcaccggat tcagtcgtca ctcatggtga tttctcactt |
| 6961 | gataacctta tttttgacga ggggaaatta ataggttgta ttgatgttgg acgagtcgga |
| 7021 | atcgcagacc gataccagga tcttgccatc ctatggaact gcctcggtga gttttctcct |
| 7081 | tcattacaga aacggctttt tcaaaaatat ggtattgata atcctgatat gaataaattg |
| 7141 | cagtttcatt tgatgctcga tgagtttttc taagcgtata atggtctaga gctagcatat |
| 7201 | ggatccatcg attccattat acgcctgtca gaccaagttt actcatatat actttagatt |
| 7261 | gatttaaaac ttcattttta atttaaaagg atctaggtga agatcctttt tgataatctc |
| 7321 | atgaccaaaa tcccttaacg tgagttttcg ttccactgag cgtcagaccc cgtagaaaag |
| 7381 | atcaaaggat cttcttgaga tccttttttt ctgcgcgtaa tctgctgctt gcaaacaaaa |
| 7441 | aaaccaccgc taccagcggt ggtttgtttg ccggatcaag agctaccaac tctttttccg |
| 7501 | aaggtaactg gcttcagcag agcgcagata ccaaatactg ttcttctagt gtagccgtag |
| 7561 | ttaggccacc acttcaagaa ctctgtagca ccgcctacat acctcgctct gctaatcctg |
| 7621 | ttaccagtgg ctgctgccag tggcgataag tcgtgtctta ccgggttgga ctcaagacga |
| 7681 | tagttaccgg ataaggcgca gcggtcgggc tgaacggggg gttcgtgcac acagcccagc |
| 7741 | ttggagcgaa cgacctacac cgaactgaga tacctacagc gtgagctatg agaaagcgcc |
| 7801 | acgcttcccg aagggagaaa ggcggacagg tatccggtaa gcggcagggt cggaacagga |
| 7861 | gagcgcacga gggagcttcc agggggaaac gcctggtatc tttatagtcc tgtcgggttt |
| 7921 | cgccacctct gacttgagcg tcgatttttg tgatgctcgt caggggggcg gagcctatgg |
| 7981 | aaaaacgcca gcaacgcggc ctttttacgg ttcctggcct tttgctggcc ttttgctcac |
| 8041 | atgt |

| pP074_seq3-201Ig_25-56 deletion (SEQ ID NO: 479) | |
|---|---|
| (SEQ ID NO: 479) | |
| 1 | tctagagcta gcatatggat ccatcgattt agggataaca gggtaattat cagcacacaa |
| 61 | ttgcccatta tacgcgcgta taatggacta ttgtgtgctg atatctgtac acttaagggc |
| 121 | tagatcttag cttacgtcac tagagggtcc acgtttagtt tttaagatcc attgatctcc |
| 181 | taaacgctgc aagattcgca acctggtata cttagcctag gcgctaggtc ctagtgcagc |
| 241 | gggacttttt ttctaaagtc gttgagagga ggagtcgtca gaccagatag ctttgatgtc |
| 301 | ctgatcggaa ggatcgttgg cccccctgca ggcagctgtt aattaactgc gcgctcgctc |
| 361 | gctcactgag gccgcccggg caaagcccgg gcgtcgggcg acctttggtc gcccggcctc |
| 421 | agtgagcgag cgagcgcgca gagagggagt ggccaactcc atcactaggg gttccttgta |
| 481 | gttaatgatt aacccgccat gctacttatc tacggcgcgc cacgcgttct aggagttaat |
| 541 | ttttaaaaag cagtcaaaag tccaagtgcc cttgcgagca tttactctct ctgtttgctc |
| 601 | tggttaataa tctcaggagc acaaacattc cttactagtt ctaggagtta atttttaaaa |
| 661 | agcagtcaaa agtccaagtg cccttgcgag catttactct ctctgtttgc tctggttaat |
| 721 | aatctcagga gcacaaacat tccttactag ttctagagcg gccgccagtg tgctggaatt |
| 781 | cggctttttt agggctggaa gctacctttg acatcatttc ctctgcgaat gcatgtataa |
| 841 | tttctacaga acctattaga aaggatcacc cagcctctgc ttttgtacaa ctttccctta |
| 901 | aaaaactgcc aatcccactg ctgtttggcc caatagtgag aactttttcc tgctgcctct |
| 961 | tggtgctttt gcctatggcc cctattctgc ctgctgaaga cactcttgcc agcatggact |
| 1021 | taaacccctc cagctctgac aatcctcttt ctcttttgtt ttacatgaag ggtctggcag |
| 1081 | ccaaagcaat cactcaaagt tcaaacctta tcattttttg ctttgttcct cttggccttg |
| 1141 | gttttgtaca tcagctttga aaataccatc ccagggttaa tgctggggtt aatttataac |
| 1201 | tgagagtgct ctagttctgc aatacaggac atgctataaa aatggaaaga tgttgctttc |
| 1261 | tgagagatca ggtggattct tgggcatttg cttaagcttg gtaccttttt tatgggagtg |
| 1321 | agacatcctc cttgttctca tagactgctg gctgtgtgtg ctctggtgtc tctggctaca |
| 1381 | gccatggagt ttgggctgag ctgggtcttt ctggtggccc tgctgaaggg agtccagtgt |
| 1441 | gagcagcagg gagcttctag acctggacct agagatgctc aggctcatcc tggaagacct |
| 1501 | agagctgtgc ctacacagtg tgatgtgcct cctaattcta gatttgattg tgctcctgat |
| 1561 | aaggctataa cacaggagca gtgtgaggct agaggatgtt gttatattcc tgctaagcag |
| 1621 | ggactgcagg gagctcagat gggacagcct tggtgttttt ttcctccttc ttatccttct |
| 1681 | tataagctgg agaatctgtc ttcttctgag atgggatata cagctacact gacaagaaca |
| 1741 | acacctacat tttttcctaa ggatattctg acactgagac tggatgtgat gatggagaca |
| 1801 | gagaatagac tgcattttac aattaaggat cctgctaata gaagatatga ggtgcctcta |
| 1861 | gagacaccta gagtgcattc tagagctcct tctcctctgt attctgtgga gttttctgag |
| 1921 | gagccttttg gagtgattgt gcatagacag ctggatggaa gagtgctgct gaatacaaca |
| 1981 | gtggctcctc tgttttttgc tgatcagttt ctgcagctgt ctacatctct gccttctcag |
| 2041 | tatattacag gactggctga gcatctgtct cctctgatgc tgtctacatc ttggacaaga |
| 2101 | attacactgt ggaatagaga tctggctcct acacctggag ctaatctgta tggatctcat |
| 2161 | cctttttatc tggctctgga ggatggagga tctgctcatg gagtgtttct gctgaattct |
| 2221 | aatgctatgg atgtagtgct gcagccttct cctgctctgt cttggagatc tacaggagga |
| 2281 | attctggatg tgtatatttt tctaggacct gagcctaagt ctgtggtgca gcagtatctg |
| 2341 | gatgtagtgg gatatccttt tatgcctcct tattggggac tgggatttca tctgtgtaga |
| 2401 | tggggatatt cttctacagc tattacaaga caggtggttg agaatatgac aagagctcat |
| 2461 | tttcctctgg atgtgcagtg gaatgatcta gattatatgg attctagaag agattttaca |
| 2521 | tttaataagg atggatttag agattttcct gctatggtgc aggagctgca tcagggagga |
| 2581 | agaagatata tgatgattgt ggatcctgct atttcttctt ctggacctgc tggatcttat |
| 2641 | agaccttatg atgagggact gagaagagga gtgtttatta caaatgagac aggacagcct |
| 2701 | ctgattggaa aggtgtggcc tggatctaca gcttttcctg attttacaaa tcctacagct |
| 2761 | ctggcttggt gggaggatat ggtggctgag tttcatgatc aggtgccttt tgatggaatg |
| 2821 | tggattgata tgaatgagcc ttctaatttt ataagaggat ctgaggatgg atgtcctaat |
| 2881 | aatgagctag agaatcctcc ttatgtgcct ggagtagtgg gaggaacact gcaggctgct |
| 2941 | acaatttgtg cttcttctca tcagtttctg tctacacatt ataatctgca taatctgtat |
| 3001 | ggactgacag aggctattgc ttctcataga gctctggtga aggctagagg aacaagacct |
| 3061 | tttgtgattt ctagatctac atttgctgga catggaagat atgctggaca ttggacagga |
| 3121 | gatgtatggt cttcttggga gcagctagct tcttctgtgc ctgagattct gcagtttaat |
| 3181 | ctgctaggag tgcctctggt gggagctgat gtgtgtggat ttctgggaaa tacatctgag |
| 3241 | gagctgtgtg tgagatggac acagctggga gctttttatc cttttatgag aaatcataat |
| 3301 | tctctgctgt ctctgcctca ggagccttat tctttttctg agcctgctca gcaggctatg |
| 3361 | agaaaggctc tgacactgag atatgctctg ctgcctcatc tgtatacact gtttcatcag |
| 3421 | gctcatgtgg ctggagagac agtagctaga cctctgtttc tggagtttcc taaggattct |
| 3481 | tctacatgga cagttgatca tcagctgctg tggggagagg ctctgctgat tacacctgtg |
| 3541 | ctgcaggctg gaaaggctga ggtgacagga tattttcctc tgggaacatg gtatgatctg |
| 3601 | cagacagtgc ctatagaggc tctgggatct ctgcctcctc ctcctgctgc tcctagagag |
| 3661 | cctgctattc attctgaggg acagtgggtg acactgcctg ctcctctgga tacaattaat |
| 3721 | gtgcatctga gagctggata tattattcct ctgcagggac ctggactgac aacaacagag |
| 3781 | tctagacagc agcctatggc tctggctgtg gctctgacaa agggaggaga ggctagagga |
| 3841 | gagctgtttt gggatgatgg agagtctctg gaggtgctgg agagaggagc ttatacacag |
| 3901 | gtgatttttc tggctagaaa taatacaatt gtgaatgagc tggtgagagt gacatctgag |
| 3961 | ggagctggac tgcagctgca gaaggtgaca gtgctgggag tggctacagc tcctcagcag |
| 4021 | gtgctgtcta atggagtgcc tgtgtctaat tttacatatt ctcctgatac aaaggtgctg |
| 4081 | gatatttgtg tgtctctgct gatgggagag cagtttctgg tgtcttggtg ttaatgattt |
| 4141 | aaatctcgag ccgggcggag tgtgttagtc tctccagagg gaggctggtt ccccagggaa |
| 4201 | gcagagcctg tgtgcgggca gcagctgtgt gcgggcctgg gggttgttaa gtgcaattat |
| 4261 | ttttaataaa aggggcattt ggaaaaaaaa aaaaaaggta gcagtcgaca gatgaattct |
| 4321 | gcagatctgt ggcttctagc tgcccgggtg gcatccctgt gacccctccc cagtgcctct |
| 4381 | cctggccctg gaagttgcca ctccagtgcc caccagcctt gtcctaataa aattaagttg |
| 4441 | catcattttg tctgactagg tgtccttcta taatattatg gggtggaggg gggtggtatg |
| 4501 | gagcaagggg caagttggga agacaacctg tagggcctgc ggggtctatt gggaaccaag |
| 4561 | ctggagtgca gtggcacaat cttggctcac tgcaatctcc gcctcctggg ttcaagcgat |
| 4621 | tctcctgcct cagcctcccg agttgttggg attccaggca tgcatgacca ggctcagcta |
| 4681 | atttttgttt ttttggtaga gacggggttt caccatattg gccaggctgg tctccaactc |
| 4741 | ctaatctcag gtgatctacc caccttggcc tcccaaattg ctgggattac aggcgtgaac |
| 4801 | cactgctccc ttccctgtcc ttctgatttt aaaaaaaaga gaaatgttct ggcacctgca |
| 4861 | cttgcactgg ggacagccta ttttgctagt ttgttttgtt tcgttttgtt ttgatggaga |
| 4921 | gcgtatgttg tttaaacgcg gccgcgtaga taagtagcat ggcgggttaa tcattaacta |
| 4981 | caaggaaccc ctagtgatgg agttggccac tccctctctg cgcgctcgct cgctcactga |
| 5041 | ggccgggcga ccaaaggtcg cccgacgccc gggctttgcc cgggcggcct cagtgagcga |
| 5101 | gcgagcgcgc agttaattaa ggcgccctag gccgaccctt agactctgta ctcagttcta |
| 5161 | taaacgagcc attggatacg agatccgtag attgataagg gacacggaat atccccggac |
| 5221 | gcaatagaca ccggtggaca gcttggtatc ctgagcacag tcgcgcgtcc gaatctagct |
| 5281 | ctactttaga ggccccggat tctgatggtc gtagaccgca gaaccgattg gggggatgag |
| 5341 | atctactagt tatcagcaca caattgccca ttatacgcgc gtataatgga ctattgtgtg |
| 5401 | ctgatatagg gataacaggg taattctaga gctagcatat ggatccatcg atttgatgcg |
| 5461 | gtattttctc cttacgcatc tgtgcggtat ttcacaccgc atacgtcaaa gcaaccatag |
| 5521 | tacgcgccct gtagcggcgc attaagcgcg gcgggtgtgg tggttacgcg cagcgtgacc |
| 5581 | gctacacttg ccagcgccct agcgcccgct cctttcgctt tcttcccttc ctttctcgcc |
| 5641 | acgttcgccg gctttccccg tcaagctcta aatcgggggc tccctttagg gttccgattt |
| 5701 | agtgctttac ggcacctcga ccccaaaaaa cttgatttgg gtgatggttc acgtagtggg |
| 5761 | ccatcgccct gatagacggt ttttcgccct ttgacgttgg agtccacgtt ctttaatagt |
| 5821 | ggactcttgt tccaaactgg aacaacactc aactctatct cgggctattc ttttgattta |
| 5881 | taagggattt tgccgatttc ggtctattgg ttaaaaaatg agctgattta acaaaaattt |
| 5941 | aacgcgaatt ttaacaaaat attaacgttt acaattttat ggtgcactct cagtacaatc |
| 6001 | tgctctgatg ccgcatagtt aagccagccc cgacacccgc caacacccgc tgacgcgccc |
| 6061 | tgacgggctt gtctgctccc ggcatccgct tacagacaag ctgtgaccgt ctccgggagc |
| 6121 | tgcatgtgtc agaggttttc accgtcatca ccgaaacgcg cgagacgaaa gggcctcgtg |
| 6181 | atacgcctat ttttataggt taatgtcatg ataataatgg tttcttagac gtcaggtggc |
| 6241 | acttttcggg gaaatgtgcg cggaacccct atttgtttat ttttctaaat acattcaaat |
| 6301 | atgtatccgc tcatgagaca ataaccctga taaatgcttc aataatattg aaaaaggaag |
| 6361 | agtatgagcc atattcaacg ggaaacgtcg aggccgcgat taaattccaa catggatgct |
| 6421 | gatttatatg ggtataaatg ggctcgcgat aatgtcgggc aatcaggtgc gacaatctat |
| 6481 | cgcttgtatg ggaagcccga tgcgccagag ttgtttctga aacatggcaa aggtagcgtt |
| 6541 | gccaatgatg ttacagatga gatggtcaga ctaaactggc tgacggaatt tatgcctctt |
| 6601 | ccgaccatca agcattttat ccgtactcct gatgatgcat ggttactcac cactgcgatc |
| 6661 | cccggaaaaa cagcattcca ggtattagaa gaatatcctg attcaggtga aaatattgtt |
| 6721 | gatgcgctgg cagtgttcct gcgccggttg cattcgattc ctgtttgtaa ttgtcctttt |
| 6781 | aacagcgatc gcgtatttcg tctcgctcag gcgcaatcac gaatgaataa cggtttggtt |
| 6841 | gatgcgagtg attttgatga cgagcgtaat ggctggcctg ttgaacaagt ctggaaagaa |
| 6901 | atgcataaac ttttgccatt ctcaccggat tcagtcgtca ctcatggtga tttctcactt |
| 6961 | gataacctta tttttgacga ggggaaatta ataggttgta ttgatgttgg acgagtcgga |
| 7021 | atcgcagacc gataccagga tcttgccatc ctatggaact gcctcggtga gttttctcct |
| 7081 | tcattacaga aacggctttt tcaaaaatat ggtattgata atcctgatat gaataaattg |
| 7141 | cagtttcatt tgatgctcga tgagtttttc taagcgtata atggtctaga gctagcatat |
| 7201 | ggatccatcg attccattat acgcctgtca gaccaagttt actcatatat actttagatt |
| 7261 | gatttaaaac ttcattttta atttaaaagg atctaggtga agatcctttt tgataatctc |
| 7321 | atgaccaaaa tcccttaacg tgagttttcg ttccactgag cgtcagaccc cgtagaaaag |
| 7381 | atcaaaggat cttcttgaga tccttttttt ctgcgcgtaa tctgctgctt gcaaacaaaa |
| 7441 | aaaccaccgc taccagcggt ggtttgtttg ccggatcaag agctaccaac tctttttccg |
| 7501 | aaggtaactg gcttcagcag agcgcagata ccaaatactg ttcttctagt gtagccgtag |
| 7561 | ttaggccacc acttcaagaa ctctgtagca ccgcctacat acctcgctct gctaatcctg |
| 7621 | ttaccagtgg ctgctgccag tggcgataag tcgtgtctta ccgggttgga ctcaagacga |
| 7681 | tagttaccgg ataaggcgca gcggtcgggc tgaacggggg gttcgtgcac acagcccagc |
| 7741 | ttggagcgaa cgacctacac cgaactgaga tacctacagc gtgagctatg agaaagcgcc |
| 7801 | acgcttcccg aagggagaaa ggcggacagg tatccggtaa gcggcagggt cggaacagga |
| 7861 | gagcgcacga gggagcttcc agggggaaac gcctggtatc tttatagtcc tgtcgggttt |
| 7921 | cgccacctct gacttgagcg tcgatttttg tgatgctcgt caggggggcg gagcctatgg |
| 7981 | aaaaacgcca gcaacgcggc ctttttacgg ttcctggcct tttgctggcc ttttgctcac |
| 8041 | atgt |

| pP075_seq100-IL2wt_newstart | |
|---|---|
| (SEQ ID NO: 480) | |
| 1 | tctagagcta gcatatggat ccatcgattt agggataaca gggtaattat cagcacacaa |
| 61 | ttgcccatta tacgcgcgta taatggacta ttgtgtgctg atatctgtac acttaagggc |
| 121 | tagatcttag cttacgtcac tagagggtcc acgtttagtt tttaagatcc attgatctcc |
| 181 | taaacgctgc aagattcgca acctggtata cttagcctag gcgctaggtc ctagtgcagc |
| 241 | gggacttttt ttctaaagtc gttgagagga ggagtcgtca gaccagatag ctttgatgtc |
| 301 | ctgatcggaa ggatcgttgg cccccctgca ggcagctgtt aattaactgc gcgctcgctc |
| 361 | gctcactgag gccgcccggg caaagcccgg gcgtcgggcg acctttggtc gcccggcctc |
| 421 | agtgagcgag cgagcgcgca gagagggagt ggccaactcc atcactaggg gttccttgta |
| 481 | gttaatgatt aacccgccat gctacttatc tacggcgcgc cacgcgttct aggagttaat |
| 541 | ttttaaaaag cagtcaaaag tccaagtgcc cttgcgagca tttactctct ctgtttgctc |
| 601 | tggttaataa tctcaggagc acaaacattc cttactagtt ctaggagtta atttttaaaa |
| 661 | agcagtcaaa agtccaagtg cccttgcgag catttactct ctctgtttgc tctggttaat |
| 721 | aatctcagga gcacaaacat tccttactag ttctagagcg gccgccagtg tgctggaatt |
| 781 | cggctttttt agggctggaa gctacctttg acatcatttc ctctgcgaat gcatgtataa |
| 841 | tttctacaga acctattaga aaggatcacc cagcctctgc ttttgtacaa ctttccctta |
| 901 | aaaaactgcc aatcccactg ctgtttggcc caatagtgag aactttttcc tgctgcctct |
| 961 | tggtgctttt gcctatggcc cctattctgc ctgctgaaga cactcttgcc agcatggact |
| 1021 | taaacccctc cagctctgac aatcctcttt ctcttttgtt ttacatgaag ggtctggcag |
| 1081 | ccaaagcaat cactcaaagt tcaaacctta tcattttttg ctttgttcct cttggccttg |
| 1141 | gttttgtaca tcagctttga aaataccatc ccagggttaa tgctggggtt aatttataac |
| 1201 | tgagagtgct ctagttctgc aatacaggac atgctataaa aatggaaaga tgttgctttc |
| 1261 | tgagagatca ggtggattct tgggcatttg cttaagcttg gtaccttttt tatgggggtg |
| 1321 | aggcaccctc cctgttctca caggctgctg gctgtgtgtg ctctggtgtc tctggccacc |
| 1381 | atgtacagaa tgcagctgct gtcctgcatt gcactgtcac tggcactggt taccaactca |
| 1441 | cagcaggggg ccagcaggcc aggccccagg gatgcccagg cccaccctgg cagacccagg |
| 1501 | gctgtgccca cccagtgtga tgtgcctccc aacagcagat ttgactgtgc ccctgataag |
| 1561 | gccattactc aggaacagtg tgaggccagg ggctgctgct atatccctgc caagcagggc |
| 1621 | ctgcaggggg cccagatggg gcagccctgg tgcttcttcc cccccagcta cccctcttat |
| 1681 | aagctggaga atctgagcag ctctgaaatg ggctacactg ccactctgac caggactacc |
| 1741 | cccaccttct tccccaagga tattctgact ctgaggctgg atgtgatgat ggagactgag |
| 1801 | aacaggctgc acttcaccat caaggaccct gccaacagga ggtatgaggt gcccctggaa |
| 1861 | actcccaggg tgcactctag ggcccccagc cccctgtatt ctgtggagtt ctctgaggag |
| 1921 | ccttttgggg tcattgtcca caggcagctg gatggcaggg tgctgctgaa tactactgtg |
| 1981 | gcccctctgt tctttgctga ccagttcctg cagctgagca ccagcctgcc ttctcagtac |
| 2041 | attactggcc tggctgagca tctgagcccc ctgatgctga gcacctcttg gaccagaatc |
| 2101 | accctgtgga acagggacct ggctcccact cctggggcca acctgtatgg cagccacccc |
| 2161 | ttctacctgg ccctggagga tgggggctct gcccatgggg tgttcctgct gaacagcaat |
| 2221 | gccatggatg tggtgctgca gccctctcct gccctgtctt ggagatctac tgggggcatc |
| 2281 | ctggatgtgt atatcttcct ggggcctgag cccaagtctg tggtgcagca gtacctggat |
| 2341 | gtggtgggct accccttcat gcccccctac tggggcctgg gcttccacct gtgcaggtgg |
| 2401 | ggctactctt ctactgctat caccaggcag gtggtggaga acatgaccag ggctcacttc |
| 2461 | cctctggatg tgcagtggaa tgacctggac tacatggact ctaggagaga cttcactttt |
| 2521 | aataaggatg ggttcaggga ctttcctgcc atggtgcagg agctgcatca ggggggcagg |
| 2581 | agatatatga tgattgtgga ccctgctatt tctagctctg gccctgctgg cagctatagg |
| 2641 | ccctatgatg aggggctgag gaggggggtg ttcatcacta atgagactgg ccagcccctg |
| 2701 | attggcaagg tgtggcctgg ctctactgcc ttccctgatt tcaccaaccc cactgccctg |
| 2761 | gcctggtggg aggatatggt ggctgagttt catgaccagg tgccctttga tggcatgtgg |
| 2821 | attgacatga atgagcccag caactttatc aggggctctg aagatggctg ccccaacaat |
| 2881 | gagctggaga acccccccta tgtgcctggg gtggtggggg gcaccctgca ggctgccacc |
| 2941 | atctgtgcca gcagccacca gttcctgagc acccactata acctgcacaa cctgtatggc |
| 3001 | ctgactgagg ccattgcctc tcacagggcc ctggtgaagg ctagggggac taggcccttt |
| 3061 | gtgatcagca ggtctacttt tgctggccat ggcaggtatg ctgggcactg gactggggat |
| 3121 | gtgtggtcta gctgggagca gctggccagc tctgtgcctg agatcctgca gtttaatctg |
| 3181 | ctgggggtgc ccctggtggg ggctgatgtg tgtggcttcc tgggcaatac ctctgaggag |
| 3241 | ctgtgtgtga ggtggactca gctgggggct ttctacccct tcatgagaaa ccacaactct |
| 3301 | ctgctgagcc tgccccagga gccctattct ttttctgagc ctgcccagca ggctatgagg |
| 3361 | aaggccctga ctctgaggta tgccctgctg ccccacctgt ataccctgtt ccatcaggcc |
| 3421 | catgtggctg gggagactgt ggccagacct ctgttcctgg agttccccaa ggatagctct |
| 3481 | acttggactg tggaccacca gctgctgtgg ggggaggctc tgctgatcac ccctgtgctg |
| 3541 | caggctggga aggctgaggt gactggctat ttccccctgg gcacctggta tgatctgcag |
| 3601 | actgtgccca ttgaggccct gggctctctg ccccctcccc ctgctgcccc cagggagcct |
| 3661 | gccatccact ctgagggcca gtgggtgacc ctgcctgccc ctctggacac tatcaatgtg |
| 3721 | cacctgaggg ctggctacat catccccctg cagggccctg gcctgactac cactgagtct |
| 3781 | aggcagcagc ccatggccct ggctgtggct ctgaccaagg ggggggaggc caggggggag |
| 3841 | ctgttctggg atgatgggga gtctctggag gtgctggaga ggggggccta cacccaggtg |
| 3901 | atcttcctgg ctaggaataa caccattgtc aatgagctgg tgagggtgac ctctgagggg |
| 3961 | gctggcctgc agctgcagaa ggtgactgtg ctgggggtgg ctactgcccc ccagcaggtg |
| 4021 | ctgagcaatg gggtgcctgt gagcaacttc acctatagcc ctgacaccaa ggtgctggac |
| 4081 | atctgtgtga gcctgctgat gggggagcag ttcctggtga gctggtgcta atttaaatct |
| 4141 | cgagccgggc ggagtgtgtt agtctctcca gagggaggct ggttccccag ggaagcagag |
| 4201 | cctgtgtgcg ggcagcagct gtgtgcgggc ctgggggttg ttaagtgcaa ttatttttaa |
| 4261 | taaaaggggc atttggaaaa aaaaaaaaaa ggtagcagtc gacagatgaa ttctgcagat |
| 4321 | ctgtggcttc tagctgcccg ggtggcatcc ctgtgacccc tccccagtgc ctctcctggc |
| 4381 | cctggaagtt gccactccag tgcccaccag ccttgtccta ataaaattaa gttgcatcat |
| 4441 | tttgtctgac taggtgtcct tctataatat tatggggtgg aggggggtgg tatggagcaa |
| 4501 | ggggcaagtt gggaagacaa cctgtagggc ctgcggggtc tattgggaac caagctggag |
| 4561 | tgcagtggca caatcttggc tcactgcaat ctccgcctcc tgggttcaag cgattctcct |
| 4621 | gcctcagcct cccgagttgt tgggattcca ggcatgcatg accaggctca gctaattttt |
| 4681 | gtttttttgg tagagacggg gtttcaccat attggccagg ctggtctcca actcctaatc |
| 4741 | tcaggtgatc tacccacctt ggcctcccaa attgctggga ttacaggcgt gaaccactgc |
| 4801 | tcccttccct gtccttctga ttttaaaaaa aagagaaatg ttctggcacc tgcacttgca |
| 4861 | ctggggacag cctattttgc tagtttgttt tgtttcgttt tgttttgatg gagagcgtat |
| 4921 | gttgtttaaa cgcggccgcg tagataagta gcatggcggg ttaatcatta actacaagga |
| 4981 | acccctagtg atggagttgg ccactccctc tctgcgcgct cgctcgctca ctgaggccgg |
| 5041 | gcgaccaaag gtcgcccgac gcccgggctt tgcccgggcg gcctcagtga gcgagcgagc |
| 5101 | gcgcagttaa ttaaggcgcc ctaggccgac ccttagactc tgtactcagt tctataaacg |
| 5161 | agccattgga tacgagatcc gtagattgat aagggacacg gaatatcccc ggacgcaata |
| 5221 | gacaccggtg gacagcttgg tatcctgagc acagtcgcgc gtccgaatct agctctactt |
| 5281 | tagaggcccc ggattctgat ggtcgtagac cgcagaaccg attgggggga tgagatctac |
| 5341 | tagttatcag cacacaattg cccattatac gcgcgtataa tggactattg tgtgctgata |
| 5401 | tagggataac agggtaattc tagagctagc atatggatcc atcgatttga tgcggtattt |
| 5461 | tctccttacg catctgtgcg gtatttcaca ccgcatacgt caaagcaacc atagtacgcg |
| 5521 | ccctgtagcg gcgcattaag cgcggcgggt gtggtggtta cgcgcagcgt gaccgctaca |
| 5581 | cttgccagcg ccctagcgcc cgctcctttc gctttcttcc cttcctttct cgccacgttc |
| 5641 | gccggctttc cccgtcaagc tctaaatcgg gggctccctt tagggttccg atttagtgct |
| 5701 | ttacggcacc tcgaccccaa aaaacttgat ttgggtgatg gttcacgtag tgggccatcg |
| 5761 | ccctgataga cggtttttcg ccctttgacg ttggagtcca cgttctttaa tagtggactc |
| 5821 | ttgttccaaa ctggaacaac actcaactct atctcgggct attcttttga tttataaggg |
| 5881 | attttgccga tttcggtcta ttggttaaaa aatgagctga tttaacaaaa atttaacgcg |
| 5941 | aattttaaca aaatattaac gtttacaatt ttatggtgca ctctcagtac aatctgctct |
| 6001 | gatgccgcat agttaagcca gccccgacac ccgccaacac ccgctgacgc gccctgacgg |
| 6061 | gcttgtctgc tcccggcatc cgcttacaga caagctgtga ccgtctccgg gagctgcatg |
| 6121 | tgtcagaggt tttcaccgtc atcaccgaaa cgcgcgagac gaaagggcct cgtgatacgc |
| 6181 | ctatttttat aggttaatgt catgataata atggtttctt agacgtcagg tggcactttt |
| 6241 | cggggaaatg tgcgcggaac ccctatttgt ttatttttct aaatacattc aaatatgtat |
| 6301 | ccgctcatga gacaataacc ctgataaatg cttcaataat attgaaaaag gaagagtatg |
| 6361 | agccatattc aacgggaaac gtcgaggccg cgattaaatt ccaacatgga tgctgattta |
| 6421 | tatgggtata aatgggctcg cgataatgtc gggcaatcag gtgcgacaat ctatcgcttg |
| 6481 | tatgggaagc ccgatgcgcc agagttgttt ctgaaacatg gcaaaggtag cgttgccaat |
| 6541 | gatgttacag atgagatggt cagactaaac tggctgacgg aatttatgcc tcttccgacc |
| 6601 | atcaagcatt ttatccgtac tcctgatgat gcatggttac tcaccactgc gatccccgga |
| 6661 | aaaacagcat tccaggtatt agaagaatat cctgattcag gtgaaaatat tgttgatgcg |
| 6721 | ctggcagtgt tcctgcgccg gttgcattcg attcctgttt gtaattgtcc ttttaacagc |
| 6781 | gatcgcgtat ttcgtctcgc tcaggcgcaa tcacgaatga ataacggttt ggttgatgcg |
| 6841 | agtgattttg atgacgagcg taatggctgg cctgttgaac aagtctggaa agaaatgcat |
| 6901 | aaacttttgc cattctcacc ggattcagtc gtcactcatg gtgatttctc acttgataac |
| 6961 | cttatttttg acgaggggaa attaataggt tgtattgatg ttggacgagt cggaatcgca |
| 7021 | gaccgatacc aggatcttgc catcctatgg aactgcctcg gtgagttttc tccttcatta |
| 7081 | cagaaacggc tttttcaaaa atatggtatt gataatcctg atatgaataa attgcagttt |
| 7141 | catttgatgc tcgatgagtt tttctaagcg tataatggtc tagagctagc atatggatcc |
| 7201 | atcgattcca ttatacgcct gtcagaccaa gtttactcat atatacttta gattgattta |
| 7261 | aaacttcatt tttaatttaa aaggatctag gtgaagatcc tttttgataa tctcatgacc |
| 7321 | aaaatccctt aacgtgagtt ttcgttccac tgagcgtcag accccgtaga aaagatcaaa |
| 7381 | ggatcttctt gagatccttt ttttctgcgc gtaatctgct gcttgcaaac aaaaaaacca |
| 7441 | ccgctaccag cggtggtttg tttgccggat caagagctac caactctttt tccgaaggta |
| 7501 | actggcttca gcagagcgca gataccaaat actgttcttc tagtgtagcc gtagttaggc |
| 7561 | caccacttca agaactctgt agcaccgcct acatacctcg ctctgctaat cctgttacca |
| 7621 | gtggctgctg ccagtggcga taagtcgtgt cttaccgggt tggactcaag acgatagtta |
| 7681 | ccggataagg cgcagcggtc gggctgaacg gggggttcgt gcacacagcc cagcttggag |
| 7741 | cgaacgacct acaccgaact gagataccta cagcgtgagc tatgagaaag cgccacgctt |
| 7801 | cccgaaggga gaaaggcgga caggtatccg gtaagcggca gggtcggaac aggagagcgc |
| 7861 | acgagggagc ttccaggggg aaacgcctgg tatctttata gtcctgtcgg gtttcgccac |
| 7921 | ctctgacttg agcgtcgatt tttgtgatgc tcgtcagggg ggcggagcct atggaaaaac |
| 7981 | gccagcaacg cggccttttt acggttcctg gccttttgct ggccttttgc tcacatgt |

| pP076_seq100-201Ig_newstart (SEQ ID NO: 481) | |
|---|---|
| (SEQ ID NO: 481) | |
| 1 | tctagagcta gcatatggat ccatcgattt agggataaca gggtaattat cagcacacaa |
| 61 | ttgcccatta tacgcgcgta taatggacta ttgtgtgctg atatctgtac acttaagggc |
| 121 | tagatcttag cttacgtcac tagagggtcc acgtttagtt tttaagatcc attgatctcc |
| 181 | taaacgctgc aagattcgca acctggtata cttagcctag gcgctaggtc ctagtgcagc |
| 241 | gggacttttt ttctaaagtc gttgagagga ggagtcgtca gaccagatag ctttgatgtc |
| 301 | ctgatcggaa ggatcgttgg cccccctgca ggcagctgtt aattaactgc gcgctcgctc |
| 361 | gctcactgag gccgcccggg caaagcccgg gcgtcgggcg acctttggtc gcccggcctc |
| 421 | agtgagcgag cgagcgcgca gagagggagt ggccaactcc atcactaggg gttccttgta |
| 481 | gttaatgatt aacccgccat gctacttatc tacggcgcgc cacgcgttct aggagttaat |
| 541 | ttttaaaaag cagtcaaaag tccaagtgcc cttgcgagca tttactctct ctgtttgctc |
| 601 | tggttaataa tctcaggagc acaaacattc cttactagtt ctaggagtta atttttaaaa |
| 661 | agcagtcaaa agtccaagtg cccttgcgag catttactct ctctgtttgc tctggttaat |
| 721 | aatctcagga gcacaaacat tccttactag ttctagagcg gccgccagtg tgctggaatt |
| 781 | cggctttttt agggctggaa gctacctttg acatcatttc ctctgcgaat gcatgtataa |
| 841 | tttctacaga acctattaga aaggatcacc cagcctctgc ttttgtacaa ctttccctta |
| 901 | aaaaactgcc aatcccactg ctgtttggcc caatagtgag aactttttcc tgctgcctct |
| 961 | tggtgctttt gcctatggcc cctattctgc ctgctgaaga cactcttgcc agcatggact |
| 1021 | taaacccctc cagctctgac aatcctcttt ctcttttgtt ttacatgaag ggtctggcag |
| 1081 | ccaaagcaat cactcaaagt tcaaacctta tcattttttg ctttgttcct cttggccttg |
| 1141 | gttttgtaca tcagctttga aaataccatc ccagggttaa tgctggggtt aatttataac |
| 1201 | tgagagtgct ctagttctgc aatacaggac atgctataaa aatggaaaga tgttgctttc |
| 1261 | tgagagatca ggtggattct tgggcatttg cttaagcttg gtaccttttt tatgggggtg |
| 1321 | aggcaccctc cctgttctca caggctgctg gctgtgtgtg ctctggtgtc tctggccacc |
| 1381 | atggagtttg ggctgagctg ggtctttctg gtggccctgc tgaagggagt ccagtgtgag |
| 1441 | cagcaggggg ccagcaggcc aggccccagg gatgcccagg cccaccctgg cagacccagg |
| 1501 | gctgtgccca cccagtgtga tgtgcctccc aacagcagat ttgactgtgc ccctgataag |
| 1561 | gccattactc aggaacagtg tgaggccagg ggctgctgct atatccctgc caagcagggc |
| 1621 | ctgcaggggg cccagatggg gcagccctgg tgcttcttcc cccccagcta cccctcttat |
| 1681 | aagctggaga atctgagcag ctctgaaatg ggctacactg ccactctgac caggactacc |
| 1741 | cccaccttct tccccaagga tattctgact ctgaggctgg atgtgatgat ggagactgag |
| 1801 | aacaggctgc acttcaccat caaggaccct gccaacagga ggtatgaggt gcccctggaa |
| 1861 | actcccaggg tgcactctag ggcccccagc cccctgtatt ctgtggagtt ctctgaggag |
| 1921 | ccttttgggg tcattgtcca caggcagctg gatggcaggg tgctgctgaa tactactgtg |
| 1981 | gcccctctgt tctttgctga ccagttcctg cagctgagca ccagcctgcc ttctcagtac |
| 2041 | attactggcc tggctgagca tctgagcccc ctgatgctga gcacctcttg gaccagaatc |
| 2101 | accctgtgga acagggacct ggctcccact cctggggcca acctgtatgg cagccacccc |
| 2161 | ttctacctgg ccctggagga tgggggctct gcccatgggg tgttcctgct gaacagcaat |
| 2221 | gccatggatg tggtgctgca gccctctcct gccctgtctt ggagatctac tgggggcatc |
| 2281 | ctggatgtgt atatcttcct ggggcctgag cccaagtctg tggtgcagca gtacctggat |
| 2341 | gtggtgggct accccttcat gcccccctac tggggcctgg gcttccacct gtgcaggtgg |
| 2401 | ggctactctt ctactgctat caccaggcag gtggtggaga acatgaccag ggctcacttc |
| 2461 | cctctggatg tgcagtggaa tgacctggac tacatggact ctaggagaga cttcactttt |
| 2521 | aataaggatg ggttcaggga ctttcctgcc atggtgcagg agctgcatca ggggggcagg |
| 2581 | agatatatga tgattgtgga ccctgctatt tctagctctg gccctgctgg cagctatagg |
| 2641 | ccctatgatg aggggctgag gaggggggtg ttcatcacta atgagactgg ccagcccctg |
| 2701 | attggcaagg tgtggcctgg ctctactgcc ttccctgatt tcaccaaccc cactgccctg |
| 2761 | gcctggtggg aggatatggt ggctgagttt catgaccagg tgccctttga tggcatgtgg |
| 2821 | attgacatga atgagcccag caactttatc aggggctctg aagatggctg ccccaacaat |
| 2881 | gagctggaga acccccccta tgtgcctggg gtggtggggg gcaccctgca ggctgccacc |
| 2941 | atctgtgcca gcagccacca gttcctgagc acccactata acctgcacaa cctgtatggc |
| 3001 | ctgactgagg ccattgcctc tcacagggcc ctggtgaagg ctagggggac taggcccttt |
| 3061 | gtgatcagca ggtctacttt tgctggccat ggcaggtatg ctgggcactg gactggggat |
| 3121 | gtgtggtcta gctgggagca gctggccagc tctgtgcctg agatcctgca gtttaatctg |
| 3181 | ctgggggtgc ccctggtggg ggctgatgtg tgtggcttcc tgggcaatac ctctgaggag |
| 3241 | ctgtgtgtga ggtggactca gctgggggct ttctacccct tcatgagaaa ccacaactct |
| 3301 | ctgctgagcc tgccccagga gccctattct ttttctgagc ctgcccagca ggctatgagg |
| 3361 | aaggccctga ctctgaggta tgccctgctg ccccacctgt ataccctgtt ccatcaggcc |
| 3421 | catgtggctg gggagactgt ggccagacct ctgttcctgg agttccccaa ggatagctct |
| 3481 | acttggactg tggaccacca gctgctgtgg ggggaggctc tgctgatcac ccctgtgctg |
| 3541 | caggctggga aggctgaggt gactggctat ttccccctgg gcacctggta tgatctgcag |
| 3601 | actgtgccca ttgaggccct gggctctctg ccccctcccc ctgctgcccc cagggagcct |
| 3661 | gccatccact ctgagggcca gtgggtgacc ctgcctgccc ctctggacac tatcaatgtg |
| 3721 | cacctgaggg ctggctacat catccccctg cagggccctg gcctgactac cactgagtct |
| 3781 | aggcagcagc ccatggccct ggctgtggct ctgaccaagg ggggggaggc caggggggag |
| 3841 | ctgttctggg atgatgggga gtctctggag gtgctggaga ggggggccta cacccaggtg |
| 3901 | atcttcctgg ctaggaataa caccattgtc aatgagctgg tgagggtgac ctctgagggg |
| 3961 | gctggcctgc agctgcagaa ggtgactgtg ctgggggtgg ctactgcccc ccagcaggtg |
| 4021 | ctgagcaatg gggtgcctgt gagcaacttc acctatagcc ctgacaccaa ggtgctggac |
| 4081 | atctgtgtga gcctgctgat gggggagcag ttcctggtga gctggtgcta atttaaatct |
| 4141 | cgagccgggc ggagtgtgtt agtctctcca gagggaggct ggttccccag ggaagcagag |
| 4201 | cctgtgtgcg ggcagcagct gtgtgcgggc ctgggggttg ttaagtgcaa ttatttttaa |
| 4261 | taaaaggggc atttggaaaa aaaaaaaaaa ggtagcagtc gacagatgaa ttctgcagat |
| 4321 | ctgtggcttc tagctgcccg ggtggcatcc ctgtgacccc tccccagtgc ctctcctggc |
| 4381 | cctggaagtt gccactccag tgcccaccag ccttgtccta ataaaattaa gttgcatcat |
| 4441 | tttgtctgac taggtgtcct tctataatat tatggggtgg aggggggtgg tatggagcaa |
| 4501 | ggggcaagtt gggaagacaa cctgtagggc ctgcggggtc tattgggaac caagctggag |
| 4561 | tgcagtggca caatcttggc tcactgcaat ctccgcctcc tgggttcaag cgattctcct |
| 4621 | gcctcagcct cccgagttgt tgggattcca ggcatgcatg accaggctca gctaattttt |
| 4681 | gtttttttgg tagagacggg gtttcaccat attggccagg ctggtctcca actcctaatc |
| 4741 | tcaggtgatc tacccacctt ggcctcccaa attgctggga ttacaggcgt gaaccactgc |
| 4801 | tcccttccct gtccttctga ttttaaaaaa aagagaaatg ttctggcacc tgcacttgca |
| 4861 | ctggggacag cctattttgc tagtttgttt tgtttcgttt tgttttgatg gagagcgtat |
| 4921 | gttgtttaaa cgcggccgcg tagataagta gcatggcggg ttaatcatta actacaagga |
| 4981 | acccctagtg atggagttgg ccactccctc tctgcgcgct cgctcgctca ctgaggccgg |
| 5041 | gcgaccaaag gtcgcccgac gcccgggctt tgcccgggcg gcctcagtga gcgagcgagc |
| 5101 | gcgcagttaa ttaaggcgcc ctaggccgac ccttagactc tgtactcagt tctataaacg |
| 5161 | agccattgga tacgagatcc gtagattgat aagggacacg gaatatcccc ggacgcaata |
| 5221 | gacaccggtg gacagcttgg tatcctgagc acagtcgcgc gtccgaatct agctctactt |
| 5281 | tagaggcccc ggattctgat ggtcgtagac cgcagaaccg attgggggga tgagatctac |
| 5341 | tagttatcag cacacaattg cccattatac gcgcgtataa tggactattg tgtgctgata |
| 5401 | tagggataac agggtaattc tagagctagc atatggatcc atcgatttga tgcggtattt |
| 5461 | tctccttacg catctgtgcg gtatttcaca ccgcatacgt caaagcaacc atagtacgcg |
| 5521 | ccctgtagcg gcgcattaag cgcggcgggt gtggtggtta cgcgcagcgt gaccgctaca |
| 5581 | cttgccagcg ccctagcgcc cgctcctttc gctttcttcc cttcctttct cgccacgttc |
| 5641 | gccggctttc cccgtcaagc tctaaatcgg gggctccctt tagggttccg atttagtgct |
| 5701 | ttacggcacc tcgaccccaa aaaacttgat ttgggtgatg gttcacgtag tgggccatcg |
| 5761 | ccctgataga cggtttttcg ccctttgacg ttggagtcca cgttctttaa tagtggactc |
| 5821 | ttgttccaaa ctggaacaac actcaactct atctcgggct attcttttga tttataaggg |
| 5881 | attttgccga tttcggtcta ttggttaaaa aatgagctga tttaacaaaa atttaacgcg |
| 5941 | aattttaaca aaatattaac gtttacaatt ttatggtgca ctctcagtac aatctgctct |
| 6001 | gatgccgcat agttaagcca gccccgacac ccgccaacac ccgctgacgc gccctgacgg |
| 6061 | gcttgtctgc tcccggcatc cgcttacaga caagctgtga ccgtctccgg gagctgcatg |
| 6121 | tgtcagaggt tttcaccgtc atcaccgaaa cgcgcgagac gaaagggcct cgtgatacgc |
| 6181 | ctatttttat aggttaatgt catgataata atggtttctt agacgtcagg tggcactttt |
| 6241 | cggggaaatg tgcgcggaac ccctatttgt ttatttttct aaatacattc aaatatgtat |
| 6301 | ccgctcatga gacaataacc ctgataaatg cttcaataat attgaaaaag gaagagtatg |
| 6361 | agccatattc aacgggaaac gtcgaggccg cgattaaatt ccaacatgga tgctgattta |
| 6421 | tatgggtata aatgggctcg cgataatgtc gggcaatcag gtgcgacaat ctatcgcttg |
| 6481 | tatgggaagc ccgatgcgcc agagttgttt ctgaaacatg gcaaaggtag cgttgccaat |
| 6541 | gatgttacag atgagatggt cagactaaac tggctgacgg aatttatgcc tcttccgacc |
| 6601 | atcaagcatt ttatccgtac tcctgatgat gcatggttac tcaccactgc gatccccgga |
| 6661 | aaaacagcat tccaggtatt agaagaatat cctgattcag gtgaaaatat tgttgatgcg |
| 6721 | ctggcagtgt tcctgcgccg gttgcattcg attcctgttt gtaattgtcc ttttaacagc |
| 6781 | gatcgcgtat ttcgtctcgc tcaggcgcaa tcacgaatga ataacggttt ggttgatgcg |
| 6841 | agtgattttg atgacgagcg taatggctgg cctgttgaac aagtctggaa agaaatgcat |
| 6901 | aaacttttgc cattctcacc ggattcagtc gtcactcatg gtgatttctc acttgataac |
| 6961 | cttatttttg acgaggggaa attaataggt tgtattgatg ttggacgagt cggaatcgca |
| 7021 | gaccgatacc aggatcttgc catcctatgg aactgcctcg gtgagttttc tccttcatta |
| 7081 | cagaaacggc tttttcaaaa atatggtatt gataatcctg atatgaataa attgcagttt |
| 7141 | catttgatgc tcgatgagtt tttctaagcg tataatggtc tagagctagc atatggatcc |
| 7201 | atcgattcca ttatacgcct gtcagaccaa gtttactcat atatacttta gattgattta |
| 7261 | aaacttcatt tttaatttaa aaggatctag gtgaagatcc tttttgataa tctcatgacc |
| 7321 | aaaatccctt aacgtgagtt ttcgttccac tgagcgtcag accccgtaga aaagatcaaa |
| 7381 | ggatcttctt gagatccttt ttttctgcgc gtaatctgct gcttgcaaac aaaaaaacca |
| 7441 | ccgctaccag cggtggtttg tttgccggat caagagctac caactctttt tccgaaggta |
| 7501 | actggcttca gcagagcgca gataccaaat actgttcttc tagtgtagcc gtagttaggc |
| 7561 | caccacttca agaactctgt agcaccgcct acatacctcg ctctgctaat cctgttacca |
| 7621 | gtggctgctg ccagtggcga taagtcgtgt cttaccgggt tggactcaag acgatagtta |
| 7681 | ccggataagg cgcagcggtc gggctgaacg gggggttcgt gcacacagcc cagcttggag |
| 7741 | cgaacgacct acaccgaact gagataccta cagcgtgagc tatgagaaag cgccacgctt |
| 7801 | cccgaaggga gaaaggcgga caggtatccg gtaagcggca gggtcggaac aggagagcgc |
| 7861 | acgagggagc ttccaggggg aaacgcctgg tatctttata gtcctgtcgg gtttcgccac |
| 7921 | ctctgacttg agcgtcgatt tttgtgatgc tcgtcagggg ggcggagcct atggaaaaac |
| 7981 | gccagcaacg cggccttttt acggttcctg gccttttgct ggccttttgc tcacatgt |

| pP077_seq3-IL2wt_newstart (SEQ ID NO: 482) | |
|---|---|
| (SEQ ID NO: 482) | |
| 1 | tctagagcta gcatatggat ccatcgattt agggataaca gggtaattat cagcacacaa |
| 61 | ttgcccatta tacgcgcgta taatggacta ttgtgtgctg atatctgtac acttaagggc |
| 121 | tagatcttag cttacgtcac tagagggtcc acgtttagtt tttaagatcc attgatctcc |
| 181 | taaacgctgc aagattcgca acctggtata cttagcctag gcgctaggtc ctagtgcagc |
| 241 | gggacttttt ttctaaagtc gttgagagga ggagtcgtca gaccagatag ctttgatgtc |
| 301 | ctgatcggaa ggatcgttgg cccccctgca ggcagctgtt aattaactgc gcgctcgctc |
| 361 | gctcactgag gccgcccggg caaagcccgg gcgtcgggcg acctttggtc gcccggcctc |
| 421 | agtgagcgag cgagcgcgca gagagggagt ggccaactcc atcactaggg gttccttgta |
| 481 | gttaatgatt aacccgccat gctacttatc tacggcgcgc cacgcgttct aggagttaat |
| 541 | ttttaaaaag cagtcaaaag tccaagtgcc cttgcgagca tttactctct ctgtttgctc |
| 601 | tggttaataa tctcaggagc acaaacattc cttactagtt ctaggagtta atttttaaaa |
| 661 | agcagtcaaa agtccaagtg cccttgcgag catttactct ctctgtttgc tctggttaat |
| 721 | aatctcagga gcacaaacat tccttactag ttctagagcg gccgccagtg tgctggaatt |
| 781 | cggctttttt agggctggaa gctacctttg acatcatttc ctctgcgaat gcatgtataa |
| 841 | tttctacaga acctattaga aaggatcacc cagcctctgc ttttgtacaa ctttccctta |
| 901 | aaaaactgcc aatcccactg ctgtttggcc caatagtgag aactttttcc tgctgcctct |
| 961 | tggtgctttt gcctatggcc cctattctgc ctgctgaaga cactcttgcc agcatggact |
| 1021 | taaacccctc cagctctgac aatcctcttt ctcttttgtt ttacatgaag ggtctggcag |
| 1081 | ccaaagcaat cactcaaagt tcaaacctta tcattttttg ctttgttcct cttggccttg |
| 1141 | gttttgtaca tcagctttga aaataccatc ccagggttaa tgctggggtt aatttataac |
| 1201 | tgagagtgct ctagttctgc aatacaggac atgctataaa aatggaaaga tgttgctttc |
| 1261 | tgagagatca ggtggattct tgggcatttg cttaagcttg gtaccttttt tatgggagtg |
| 1321 | agacatcctc cttgttctca tagactgctg gctgtgtgtg ctctggtgtc tctggccacc |
| 1381 | atgtacagaa tgcagctgct gtcctgcatt gcactgtcac tggcactggt taccaactca |
| 1441 | cagcagggag cttctagacc tggacctaga gatgctcagg ctcatcctgg aagacctaga |
| 1501 | gctgtgccta cacagtgtga tgtgcctcct aattctagat ttgattgtgc tcctgataag |
| 1561 | gctataacac aggagcagtg tgaggctaga ggatgttgtt atattcctgc taagcaggga |
| 1621 | ctgcagggag ctcagatggg acagccttgg tgtttttttc ctccttctta tccttcttat |
| 1681 | aagctggaga atctgtcttc ttctgagatg ggatatacag ctacactgac aagaacaaca |
| 1741 | cctacatttt ttcctaagga tattctgaca ctgagactgg atgtgatgat ggagacagag |
| 1801 | aatagactgc attttacaat taaggatcct gctaatagaa gatatgaggt gcctctagag |
| 1861 | acacctagag tgcattctag agctccttct cctctgtatt ctgtggagtt ttctgaggag |
| 1921 | ccttttggag tgattgtgca tagacagctg gatggaagag tgctgctgaa tacaacagtg |
| 1981 | gctcctctgt tttttgctga tcagtttctg cagctgtcta catctctgcc ttctcagtat |
| 2041 | attacaggac tggctgagca tctgtctcct ctgatgctgt ctacatcttg gacaagaatt |
| 2101 | acactgtgga atagagatct ggctcctaca cctggagcta atctgtatgg atctcatcct |
| 2161 | ttttatctgg ctctggagga tggaggatct gctcatggag tgtttctgct gaattctaat |
| 2221 | gctatggatg tagtgctgca gccttctcct gctctgtctt ggagatctac aggaggaatt |
| 2281 | ctggatgtgt atatttttct aggacctgag cctaagtctg tggtgcagca gtatctggat |
| 2341 | gtagtgggat atccttttat gcctccttat tggggactgg gatttcatct gtgtagatgg |
| 2401 | ggatattctt ctacagctat tacaagacag gtggttgaga atatgacaag agctcatttt |
| 2461 | cctctggatg tgcagtggaa tgatctagat tatatggatt ctagaagaga ttttacattt |
| 2521 | aataaggatg gatttagaga ttttcctgct atggtgcagg agctgcatca gggaggaaga |
| 2581 | agatatatga tgattgtgga tcctgctatt tcttcttctg gacctgctgg atcttataga |
| 2641 | ccttatgatg agggactgag aagaggagtg tttattacaa atgagacagg acagcctctg |
| 2701 | attggaaagg tgtggcctgg atctacagct tttcctgatt ttacaaatcc tacagctctg |
| 2761 | gcttggtggg aggatatggt ggctgagttt catgatcagg tgccttttga tggaatgtgg |
| 2821 | attgatatga atgagccttc taattttata agaggatctg aggatggatg tcctaataat |
| 2881 | gagctagaga atcctcctta tgtgcctgga gtagtgggag gaacactgca ggctgctaca |
| 2941 | atttgtgctt cttctcatca gtttctgtct acacattata atctgcataa tctgtatgga |
| 3001 | ctgacagagg ctattgcttc tcatagagct ctggtgaagg ctagaggaac aagacctttt |
| 3061 | gtgatttcta gatctacatt tgctggacat ggaagatatg ctggacattg gacaggagat |
| 3121 | gtatggtctt cttgggagca gctagcttct tctgtgcctg agattctgca gtttaatctg |
| 3181 | ctaggagtgc ctctggtggg agctgatgtg tgtggatttc tgggaaatac atctgaggag |
| 3241 | ctgtgtgtga gatggacaca gctgggagct ttttatcctt ttatgagaaa tcataattct |
| 3301 | ctgctgtctc tgcctcagga gccttattct ttttctgagc ctgctcagca ggctatgaga |
| 3361 | aaggctctga cactgagata tgctctgctg cctcatctgt atacactgtt tcatcaggct |
| 3421 | catgtggctg gagagacagt agctagacct ctgtttctgg agtttcctaa ggattcttct |
| 3481 | acatggacag ttgatcatca gctgctgtgg ggagaggctc tgctgattac acctgtgctg |
| 3541 | caggctggaa aggctgaggt gacaggatat tttcctctgg gaacatggta tgatctgcag |
| 3601 | acagtgccta tagaggctct gggatctctg cctcctcctc ctgctgctcc tagagagcct |
| 3661 | gctattcatt ctgagggaca gtgggtgaca ctgcctgctc ctctggatac aattaatgtg |
| 3721 | catctgagag ctggatatat tattcctctg cagggacctg gactgacaac aacagagtct |
| 3781 | agacagcagc ctatggctct ggctgtggct ctgacaaagg gaggagaggc tagaggagag |
| 3841 | ctgttttggg atgatggaga gtctctggag gtgctggaga gaggagctta tacacaggtg |
| 3901 | atttttctgg ctagaaataa tacaattgtg aatgagctgg tgagagtgac atctgaggga |
| 3961 | gctggactgc agctgcagaa ggtgacagtg ctgggagtgg ctacagctcc tcagcaggtg |
| 4021 | ctgtctaatg gagtgcctgt gtctaatttt acatattctc ctgatacaaa ggtgctggat |
| 4081 | atttgtgtgt ctctgctgat gggagagcag tttctggtgt cttggtgtta atgatttaaa |
| 4141 | tctcgagccg ggcggagtgt gttagtctct ccagagggag gctggttccc cagggaagca |
| 4201 | gagcctgtgt gcgggcagca gctgtgtgcg ggcctggggg ttgttaagtg caattatttt |
| 4261 | taataaaagg ggcatttgga aaaaaaaaaa aaaggtagca gtcgacagat gaattctgca |
| 4321 | gatctgtggc ttctagctgc ccgggtggca tccctgtgac ccctccccag tgcctctcct |
| 4381 | ggccctggaa gttgccactc cagtgcccac cagccttgtc ctaataaaat taagttgcat |
| 4441 | cattttgtct gactaggtgt ccttctataa tattatgggg tggagggggg tggtatggag |
| 4501 | caaggggcaa gttgggaaga caacctgtag ggcctgcggg gtctattggg aaccaagctg |
| 4561 | gagtgcagtg gcacaatctt ggctcactgc aatctccgcc tcctgggttc aagcgattct |
| 4621 | cctgcctcag cctcccgagt tgttgggatt ccaggcatgc atgaccaggc tcagctaatt |
| 4681 | tttgtttttt tggtagagac ggggtttcac catattggcc aggctggtct ccaactccta |
| 4741 | atctcaggtg atctacccac cttggcctcc caaattgctg ggattacagg cgtgaaccac |
| 4801 | tgctcccttc cctgtccttc tgattttaaa aaaaagagaa atgttctggc acctgcactt |
| 4861 | gcactgggga cagcctattt tgctagtttg ttttgtttcg ttttgttttg atggagagcg |
| 4921 | tatgttgttt aaacgcggcc gcgtagataa gtagcatggc gggttaatca ttaactacaa |
| 4981 | ggaaccccta gtgatggagt tggccactcc ctctctgcgc gctcgctcgc tcactgaggc |
| 5041 | cgggcgacca aaggtcgccc gacgcccggg ctttgcccgg gcggcctcag tgagcgagcg |
| 5101 | agcgcgcagt taattaaggc gccctaggcc gacccttaga ctctgtactc agttctataa |
| 5161 | acgagccatt ggatacgaga tccgtagatt gataagggac acggaatatc cccggacgca |
| 5221 | atagacaccg gtggacagct tggtatcctg agcacagtcg cgcgtccgaa tctagctcta |
| 5281 | ctttagaggc cccggattct gatggtcgta gaccgcagaa ccgattgggg ggatgagatc |
| 5341 | tactagttat cagcacacaa ttgcccatta tacgcgcgta taatggacta ttgtgtgctg |
| 5401 | atatagggat aacagggtaa ttctagagct agcatatgga tccatcgatt tgatgcggta |
| 5461 | ttttctcctt acgcatctgt gcggtatttc acaccgcata cgtcaaagca accatagtac |
| 5521 | gcgccctgta gcggcgcatt aagcgcggcg ggtgtggtgg ttacgcgcag cgtgaccgct |
| 5581 | acacttgcca gcgccctagc gcccgctcct ttcgctttct tcccttcctt tctcgccacg |
| 5641 | ttcgccggct ttccccgtca agctctaaat cgggggctcc ctttagggtt ccgatttagt |
| 5701 | gctttacggc acctcgaccc caaaaaactt gatttgggtg atggttcacg tagtgggcca |
| 5761 | tcgccctgat agacggtttt tcgccctttg acgttggagt ccacgttctt taatagtgga |
| 5821 | ctcttgttcc aaactggaac aacactcaac tctatctcgg gctattcttt tgatttataa |
| 5881 | gggattttgc cgatttcggt ctattggtta aaaaatgagc tgatttaaca aaaatttaac |
| 5941 | gcgaatttta acaaaatatt aacgtttaca attttatggt gcactctcag tacaatctgc |
| 6001 | tctgatgccg catagttaag ccagccccga cacccgccaa cacccgctga cgcgccctga |
| 6061 | cgggcttgtc tgctcccggc atccgcttac agacaagctg tgaccgtctc cgggagctgc |
| 6121 | atgtgtcaga ggttttcacc gtcatcaccg aaacgcgcga gacgaaaggg cctcgtgata |
| 6181 | cgcctatttt tataggttaa tgtcatgata ataatggttt cttagacgtc aggtggcact |
| 6241 | tttcggggaa atgtgcgcgg aacccctatt tgtttatttt tctaaataca ttcaaatatg |
| 6301 | tatccgctca tgagacaata accctgataa atgcttcaat aatattgaaa aaggaagagt |
| 6361 | atgagccata ttcaacggga aacgtcgagg ccgcgattaa attccaacat ggatgctgat |
| 6421 | ttatatgggt ataaatgggc tcgcgataat gtcgggcaat caggtgcgac aatctatcgc |
| 6481 | ttgtatggga agcccgatgc gccagagttg tttctgaaac atggcaaagg tagcgttgcc |
| 6541 | aatgatgtta cagatgagat ggtcagacta aactggctga cggaatttat gcctcttccg |
| 6601 | accatcaagc attttatccg tactcctgat gatgcatggt tactcaccac tgcgatcccc |
| 6661 | ggaaaaacag cattccaggt attagaagaa tatcctgatt caggtgaaaa tattgttgat |
| 6721 | gcgctggcag tgttcctgcg ccggttgcat tcgattcctg tttgtaattg tccttttaac |
| 6781 | agcgatcgcg tatttcgtct cgctcaggcg caatcacgaa tgaataacgg tttggttgat |
| 6841 | gcgagtgatt ttgatgacga gcgtaatggc tggcctgttg aacaagtctg gaaagaaatg |
| 6901 | cataaacttt tgccattctc accggattca gtcgtcactc atggtgattt ctcacttgat |
| 6961 | aaccttattt ttgacgaggg gaaattaata ggttgtattg atgttggacg agtcggaatc |
| 7021 | gcagaccgat accaggatct tgccatccta tggaactgcc tcggtgagtt ttctccttca |
| 7081 | ttacagaaac ggctttttca aaaatatggt attgataatc ctgatatgaa taaattgcag |
| 7141 | tttcatttga tgctcgatga gtttttctaa gcgtataatg gtctagagct agcatatgga |
| 7201 | tccatcgatt ccattatacg cctgtcagac caagtttact catatatact ttagattgat |
| 7261 | ttaaaacttc atttttaatt taaaaggatc taggtgaaga tcctttttga taatctcatg |
| 7321 | accaaaatcc cttaacgtga gttttcgttc cactgagcgt cagaccccgt agaaaagatc |
| 7381 | aaaggatctt cttgagatcc tttttttctg cgcgtaatct gctgcttgca aacaaaaaaa |
| 7441 | ccaccgctac cagcggtggt ttgtttgccg gatcaagagc taccaactct ttttccgaag |
| 7501 | gtaactggct tcagcagagc gcagatacca aatactgttc ttctagtgta gccgtagtta |
| 7561 | ggccaccact tcaagaactc tgtagcaccg cctacatacc tcgctctgct aatcctgtta |
| 7621 | ccagtggctg ctgccagtgg cgataagtcg tgtcttaccg ggttggactc aagacgatag |
| 7681 | ttaccggata aggcgcagcg gtcgggctga acggggggtt cgtgcacaca gcccagcttg |
| 7741 | gagcgaacga cctacaccga actgagatac ctacagcgtg agctatgaga aagcgccacg |
| 7801 | cttcccgaag ggagaaaggc ggacaggtat ccggtaagcg gcagggtcgg aacaggagag |
| 7861 | cgcacgaggg agcttccagg gggaaacgcc tggtatcttt atagtcctgt cgggtttcgc |
| 7921 | cacctctgac ttgagcgtcg atttttgtga tgctcgtcag gggggcggag cctatggaaa |
| 7981 | aacgccagca acgcggcctt tttacggttc ctggcctttt gctggccttt tgctcacatg |
| 8041 | t |

| pP078_seq3-201Ig_newstart | |
|---|---|
| (SEQ ID NO: 483) | |
| 1 | tctagagcta gcatatggat ccatcgattt agggataaca gggtaattat cagcacacaa |
| 61 | ttgcccatta tacgcgcgta taatggacta ttgtgtgctg atatctgtac acttaagggc |
| 121 | tagatcttag cttacgtcac tagagggtcc acgtttagtt tttaagatcc attgatctcc |
| 181 | taaacgctgc aagattcgca acctggtata cttagcctag gcgctaggtc ctagtgcagc |
| 241 | gggacttttt ttctaaagtc gttgagagga ggagtcgtca gaccagatag ctttgatgtc |
| 301 | ctgatcggaa ggatcgttgg cccccctgca ggcagctgtt aattaactgc gcgctcgctc |
| 361 | gctcactgag gccgcccggg caaagcccgg gcgtcgggcg acctttggtc gcccggcctc |
| 421 | agtgagcgag cgagcgcgca gagagggagt ggccaactcc atcactaggg gttccttgta |
| 481 | gttaatgatt aacccgccat gctacttatc tacggcgcgc cacgcgttct aggagttaat |
| 541 | ttttaaaaag cagtcaaaag tccaagtgcc cttgcgagca tttactctct ctgtttgctc |
| 601 | tggttaataa tctcaggagc acaaacattc cttactagtt ctaggagtta atttttaaaa |
| 661 | agcagtcaaa agtccaagtg cccttgcgag catttactct ctctgtttgc tctggttaat |
| 721 | aatctcagga gcacaaacat tccttactag ttctagagcg gccgccagtg tgctggaatt |
| 781 | cggctttttt agggctggaa gctacctttg acatcatttc ctctgcgaat gcatgtataa |
| 841 | tttctacaga acctattaga aaggatcacc cagcctctgc ttttgtacaa ctttccctta |
| 901 | aaaaactgcc aatcccactg ctgtttggcc caatagtgag aactttttcc tgctgcctct |
| 961 | tggtgctttt gcctatggcc cctattctgc ctgctgaaga cactcttgcc agcatggact |
| 1021 | taaacccctc cagctctgac aatcctcttt ctcttttgtt ttacatgaag ggtctggcag |
| 1081 | ccaaagcaat cactcaaagt tcaaacctta tcattttttg ctttgttcct cttggccttg |
| 1141 | gttttgtaca tcagctttga aaataccatc ccagggttaa tgctggggtt aatttataac |
| 1201 | tgagagtgct ctagttctgc aatacaggac atgctataaa aatggaaaga tgttgctttc |
| 1261 | tgagagatca ggtggattct tgggcatttg cttaagcttg gtaccttttt tatgggagtg |
| 1321 | agacatcctc cttgttctca tagactgctg gctgtgtgtg ctctggtgtc tctggccacc |
| 1381 | atggagtttg ggctgagctg ggtctttctg gtggccctgc tgaagggagt ccagtgtgag |
| 1441 | cagcagggag cttctagacc tggacctaga gatgctcagg ctcatcctgg aagacctaga |
| 1501 | gctgtgccta cacagtgtga tgtgcctcct aattctagat ttgattgtgc tcctgataag |
| 1561 | gctataacac aggagcagtg tgaggctaga ggatgttgtt atattcctgc taagcaggga |
| 1621 | ctgcagggag ctcagatggg acagccttgg tgtttttttc ctccttctta tccttcttat |
| 1681 | aagctggaga atctgtcttc ttctgagatg ggatatacag ctacactgac aagaacaaca |
| 1741 | cctacatttt ttcctaagga tattctgaca ctgagactgg atgtgatgat ggagacagag |
| 1801 | aatagactgc attttacaat taaggatcct gctaatagaa gatatgaggt gcctctagag |
| 1861 | acacctagag tgcattctag agctccttct cctctgtatt ctgtggagtt ttctgaggag |
| 1921 | ccttttggag tgattgtgca tagacagctg gatggaagag tgctgctgaa tacaacagtg |
| 1981 | gctcctctgt tttttgctga tcagtttctg cagctgtcta catctctgcc ttctcagtat |
| 2041 | attacaggac tggctgagca tctgtctcct ctgatgctgt ctacatcttg gacaagaatt |
| 2101 | acactgtgga atagagatct ggctcctaca cctggagcta atctgtatgg atctcatcct |
| 2161 | ttttatctgg ctctggagga tggaggatct gctcatggag tgtttctgct gaattctaat |
| 2221 | gctatggatg tagtgctgca gccttctcct gctctgtctt ggagatctac aggaggaatt |
| 2281 | ctggatgtgt atatttttct aggacctgag cctaagtctg tggtgcagca gtatctggat |
| 2341 | gtagtgggat atccttttat gcctccttat tggggactgg gatttcatct gtgtagatgg |
| 2401 | ggatattctt ctacagctat tacaagacag gtggttgaga atatgacaag agctcatttt |
| 2461 | cctctggatg tgcagtggaa tgatctagat tatatggatt ctagaagaga ttttacattt |
| 2521 | aataaggatg gatttagaga ttttcctgct atggtgcagg agctgcatca gggaggaaga |
| 2581 | agatatatga tgattgtgga tcctgctatt tcttcttctg gacctgctgg atcttataga |
| 2641 | ccttatgatg agggactgag aagaggagtg tttattacaa atgagacagg acagcctctg |
| 2701 | attggaaagg tgtggcctgg atctacagct tttcctgatt ttacaaatcc tacagctctg |
| 2761 | gcttggtggg aggatatggt ggctgagttt catgatcagg tgccttttga tggaatgtgg |
| 2821 | attgatatga atgagccttc taattttata agaggatctg aggatggatg tcctaataat |
| 2881 | gagctagaga atcctcctta tgtgcctgga gtagtgggag gaacactgca ggctgctaca |
| 2941 | atttgtgctt cttctcatca gtttctgtct acacattata atctgcataa tctgtatgga |
| 3001 | ctgacagagg ctattgcttc tcatagagct ctggtgaagg ctagaggaac aagacctttt |
| 3061 | gtgatttcta gatctacatt tgctggacat ggaagatatg ctggacattg gacaggagat |
| 3121 | gtatggtctt cttgggagca gctagcttct tctgtgcctg agattctgca gtttaatctg |
| 3181 | ctaggagtgc ctctggtggg agctgatgtg tgtggatttc tgggaaatac atctgaggag |
| 3241 | ctgtgtgtga gatggacaca gctgggagct ttttatcctt ttatgagaaa tcataattct |
| 3301 | ctgctgtctc tgcctcagga gccttattct ttttctgagc ctgctcagca ggctatgaga |
| 3361 | aaggctctga cactgagata tgctctgctg cctcatctgt atacactgtt tcatcaggct |
| 3421 | catgtggctg gagagacagt agctagacct ctgtttctgg agtttcctaa ggattcttct |
| 3481 | acatggacag ttgatcatca gctgctgtgg ggagaggctc tgctgattac acctgtgctg |
| 3541 | caggctggaa aggctgaggt gacaggatat tttcctctgg gaacatggta tgatctgcag |
| 3601 | acagtgccta tagaggctct gggatctctg cctcctcctc ctgctgctcc tagagagcct |
| 3661 | gctattcatt ctgagggaca gtgggtgaca ctgcctgctc ctctggatac aattaatgtg |
| 3721 | catctgagag ctggatatat tattcctctg cagggacctg gactgacaac aacagagtct |
| 3781 | agacagcagc ctatggctct ggctgtggct ctgacaaagg gaggagaggc tagaggagag |
| 3841 | ctgttttggg atgatggaga gtctctggag gtgctggaga gaggagctta tacacaggtg |
| 3901 | atttttctgg ctagaaataa tacaattgtg aatgagctgg tgagagtgac atctgaggga |
| 3961 | gctggactgc agctgcagaa ggtgacagtg ctgggagtgg ctacagctcc tcagcaggtg |
| 4021 | ctgtctaatg gagtgcctgt gtctaatttt acatattctc ctgatacaaa ggtgctggat |
| 4081 | atttgtgtgt ctctgctgat gggagagcag tttctggtgt cttggtgtta atgatttaaa |
| 4141 | tctcgagccg ggcggagtgt gttagtctct ccagagggag gctggttccc cagggaagca |
| 4201 | gagcctgtgt gcgggcagca gctgtgtgcg ggcctggggg ttgttaagtg caattatttt |
| 4261 | taataaaagg ggcatttgga aaaaaaaaaa aaaggtagca gtcgacagat gaattctgca |
| 4321 | gatctgtggc ttctagctgc ccgggtggca tccctgtgac ccctccccag tgcctctcct |
| 4381 | ggccctggaa gttgccactc cagtgcccac cagccttgtc ctaataaaat taagttgcat |
| 4441 | cattttgtct gactaggtgt ccttctataa tattatgggg tggagggggg tggtatggag |
| 4501 | caaggggcaa gttgggaaga caacctgtag ggcctgcggg gtctattggg aaccaagctg |
| 4561 | gagtgcagtg gcacaatctt ggctcactgc aatctccgcc tcctgggttc aagcgattct |
| 4621 | cctgcctcag cctcccgagt tgttgggatt ccaggcatgc atgaccaggc tcagctaatt |
| 4681 | tttgtttttt tggtagagac ggggtttcac catattggcc aggctggtct ccaactccta |
| 4741 | atctcaggtg atctacccac cttggcctcc caaattgctg ggattacagg cgtgaaccac |
| 4801 | tgctcccttc cctgtccttc tgattttaaa aaaaagagaa atgttctggc acctgcactt |
| 4861 | gcactgggga cagcctattt tgctagtttg ttttgtttcg ttttgttttg atggagagcg |
| 4921 | tatgttgttt aaacgcggcc gcgtagataa gtagcatggc gggttaatca ttaactacaa |
| 4981 | ggaaccccta gtgatggagt tggccactcc ctctctgcgc gctcgctcgc tcactgaggc |
| 5041 | cgggcgacca aaggtcgccc gacgcccggg ctttgcccgg gcggcctcag tgagcgagcg |
| 5101 | agcgcgcagt taattaaggc gccctaggcc gacccttaga ctctgtactc agttctataa |
| 5161 | acgagccatt ggatacgaga tccgtagatt gataagggac acggaatatc cccggacgca |
| 5221 | atagacaccg gtggacagct tggtatcctg agcacagtcg cgcgtccgaa tctagctcta |
| 5281 | ctttagaggc cccggattct gatggtcgta gaccgcagaa ccgattgggg ggatgagatc |
| 5341 | tactagttat cagcacacaa ttgcccatta tacgcgcgta taatggacta ttgtgtgctg |
| 5401 | atatagggat aacagggtaa ttctagagct agcatatgga tccatcgatt tgatgcggta |
| 5461 | ttttctcctt acgcatctgt gcggtatttc acaccgcata cgtcaaagca accatagtac |
| 5521 | gcgccctgta gcggcgcatt aagcgcggcg ggtgtggtgg ttacgcgcag cgtgaccgct |
| 5581 | acacttgcca gcgccctagc gcccgctcct ttcgctttct tcccttcctt tctcgccacg |
| 5641 | ttcgccggct ttccccgtca agctctaaat cgggggctcc ctttagggtt ccgatttagt |
| 5701 | gctttacggc acctcgaccc caaaaaactt gatttgggtg atggttcacg tagtgggcca |
| 5761 | tcgccctgat agacggtttt tcgccctttg acgttggagt ccacgttctt taatagtgga |
| 5821 | ctcttgttcc aaactggaac aacactcaac tctatctcgg gctattcttt tgatttataa |
| 5881 | gggattttgc cgatttcggt ctattggtta aaaaatgagc tgatttaaca aaaatttaac |
| 5941 | gcgaatttta acaaaatatt aacgtttaca attttatggt gcactctcag tacaatctgc |
| 6001 | tctgatgccg catagttaag ccagccccga cacccgccaa cacccgctga cgcgccctga |
| 6061 | cgggcttgtc tgctcccggc atccgcttac agacaagctg tgaccgtctc cgggagctgc |
| 6121 | atgtgtcaga ggttttcacc gtcatcaccg aaacgcgcga gacgaaaggg cctcgtgata |
| 6181 | cgcctatttt tataggttaa tgtcatgata ataatggttt cttagacgtc aggtggcact |
| 6241 | tttcggggaa atgtgcgcgg aacccctatt tgtttatttt tctaaataca ttcaaatatg |
| 6301 | tatccgctca tgagacaata accctgataa atgcttcaat aatattgaaa aaggaagagt |
| 6361 | atgagccata ttcaacggga aacgtcgagg ccgcgattaa attccaacat ggatgctgat |
| 6421 | ttatatgggt ataaatgggc tcgcgataat gtcgggcaat caggtgcgac aatctatcgc |
| 6481 | ttgtatggga agcccgatgc gccagagttg tttctgaaac atggcaaagg tagcgttgcc |
| 6541 | aatgatgtta cagatgagat ggtcagacta aactggctga cggaatttat gcctcttccg |
| 6601 | accatcaagc attttatccg tactcctgat gatgcatggt tactcaccac tgcgatcccc |
| 6661 | ggaaaaacag cattccaggt attagaagaa tatcctgatt caggtgaaaa tattgttgat |
| 6721 | gcgctggcag tgttcctgcg ccggttgcat tcgattcctg tttgtaattg tccttttaac |
| 6781 | agcgatcgcg tatttcgtct cgctcaggcg caatcacgaa tgaataacgg tttggttgat |
| 6841 | gcgagtgatt ttgatgacga gcgtaatggc tggcctgttg aacaagtctg gaaagaaatg |
| 6901 | cataaacttt tgccattctc accggattca gtcgtcactc atggtgattt ctcacttgat |
| 6961 | aaccttattt ttgacgaggg gaaattaata ggttgtattg atgttggacg agtcggaatc |
| 7021 | gcagaccgat accaggatct tgccatccta tggaactgcc tcggtgagtt ttctccttca |
| 7081 | ttacagaaac ggctttttca aaaatatggt attgataatc ctgatatgaa taaattgcag |
| 7141 | tttcatttga tgctcgatga gtttttctaa gcgtataatg gtctagagct agcatatgga |
| 7201 | tccatcgatt ccattatacg cctgtcagac caagtttact catatatact ttagattgat |
| 7261 | ttaaaacttc atttttaatt taaaaggatc taggtgaaga tcctttttga taatctcatg |
| 7321 | accaaaatcc cttaacgtga gttttcgttc cactgagcgt cagaccccgt agaaaagatc |
| 7381 | aaaggatctt cttgagatcc tttttttctg cgcgtaatct gctgcttgca aacaaaaaaa |
| 7441 | ccaccgctac cagcggtggt ttgtttgccg gatcaagagc taccaactct ttttccgaag |
| 7501 | gtaactggct tcagcagagc gcagatacca aatactgttc ttctagtgta gccgtagtta |
| 7561 | ggccaccact tcaagaactc tgtagcaccg cctacatacc tcgctctgct aatcctgtta |
| 7621 | ccagtggctg ctgccagtgg cgataagtcg tgtcttaccg ggttggactc aagacgatag |
| 7681 | ttaccggata aggcgcagcg gtcgggctga acggggggtt cgtgcacaca gcccagcttg |
| 7741 | gagcgaacga cctacaccga actgagatac ctacagcgtg agctatgaga aagcgccacg |
| 7801 | cttcccgaag ggagaaaggc ggacaggtat ccggtaagcg gcagggtcgg aacaggagag |
| 7861 | cgcacgaggg agcttccagg gggaaacgcc tggtatcttt atagtcctgt cgggtttcgc |
| 7921 | cacctctgac ttgagcgtcg atttttgtga tgctcgtcag gggggcggag cctatggaaa |
| 7981 | aacgccagca acgcggcctt tttacggttc ctggcctttt gctggccttt tgctcacatg |
| 8041 | t |

| pP079_seq100-IL2wt_3-56 deletion (SEQ ID NO: 484) | |
|---|---|
| (SEQ ID NO: 484) | |
| 1 | tctagagcta gcatatggat ccatcgattt agggataaca gggtaattat cagcacacaa |
| 61 | ttgcccatta tacgcgcgta taatggacta ttgtgtgctg atatctgtac acttaagggc |
| 121 | tagatcttag cttacgtcac tagagggtcc acgtttagtt tttaagatcc attgatctcc |
| 181 | taaacgctgc aagattcgca acctggtata cttagcctag gcgctaggtc ctagtgcagc |
| 241 | gggacttttt ttctaaagtc gttgagagga ggagtcgtca gaccagatag ctttgatgtc |
| 301 | ctgatcggaa ggatcgttgg cccccctgca ggcagctgtt aattaactgc gcgctcgctc |
| 361 | gctcactgag gccgcccggg caaagcccgg gcgtcgggcg acctttggtc gcccggcctc |
| 421 | agtgagcgag cgagcgcgca gagagggagt ggccaactcc atcactaggg gttccttgta |
| 481 | gttaatgatt aacccgccat gctacttatc tacggcgcgc cacgcgttct aggagttaat |
| 541 | ttttaaaaag cagtcaaaag tccaagtgcc cttgcgagca tttactctct ctgtttgctc |
| 601 | tggttaataa tctcaggagc acaaacattc cttactagtt ctaggagtta atttttaaaa |
| 661 | agcagtcaaa agtccaagtg cccttgcgag catttactct ctctgtttgc tctggttaat |
| 721 | aatctcagga gcacaaacat tccttactag ttctagagcg gccgccagtg tgctggaatt |
| 781 | cggctttttt agggctggaa gctacctttg acatcatttc ctctgcgaat gcatgtataa |
| 841 | tttctacaga acctattaga aaggatcacc cagcctctgc ttttgtacaa ctttccctta |
| 901 | aaaaactgcc aatcccactg ctgtttggcc caatagtgag aactttttcc tgctgcctct |
| 961 | tggtgctttt gcctatggcc cctattctgc ctgctgaaga cactcttgcc agcatggact |
| 1021 | taaacccctc cagctctgac aatcctcttt ctcttttgtt ttacatgaag ggtctggcag |
| 1081 | ccaaagcaat cactcaaagt tcaaacctta tcattttttg ctttgttcct cttggccttg |
| 1141 | gttttgtaca tcagctttga aaataccatc ccagggttaa tgctggggtt aatttataac |
| 1201 | tgagagtgct ctagttctgc aatacaggac atgctataaa aatggaaaga tgttgctttc |
| 1261 | tgagagatca ggtggattct tgggcatttg cttaagcttg gtaccgccac catggggtac |
| 1321 | agaatgcagc tgctgtcctg cattgcactg tcactggcac tggttaccaa ctcacagcag |
| 1381 | ggggccagca ggccaggccc cagggatgcc caggcccacc ctggcagacc cagggctgtg |
| 1441 | cccacccagt gtgatgtgcc tcccaacagc agatttgact gtgcccctga taaggccatt |
| 1501 | actcaggaac agtgtgaggc caggggctgc tgctatatcc ctgccaagca gggcctgcag |
| 1561 | ggggcccaga tggggcagcc ctggtgcttc ttccccccca gctacccctc ttataagctg |
| 1621 | gagaatctga gcagctctga aatgggctac actgccactc tgaccaggac tacccccacc |
| 1681 | ttcttcccca aggatattct gactctgagg ctggatgtga tgatggagac tgagaacagg |
| 1741 | ctgcacttca ccatcaagga ccctgccaac aggaggtatg aggtgcccct ggaaactccc |
| 1801 | agggtgcact ctagggcccc cagccccctg tattctgtgg agttctctga ggagcctttt |
| 1861 | ggggtcattg tccacaggca gctggatggc agggtgctgc tgaatactac tgtggcccct |
| 1921 | ctgttctttg ctgaccagtt cctgcagctg agcaccagcc tgccttctca gtacattact |
| 1981 | ggcctggctg agcatctgag ccccctgatg ctgagcacct cttggaccag aatcaccctg |
| 2041 | tggaacaggg acctggctcc cactcctggg gccaacctgt atggcagcca ccccttctac |
| 2101 | ctggccctgg aggatggggg ctctgcccat ggggtgttcc tgctgaacag caatgccatg |
| 2161 | gatgtggtgc tgcagccctc tcctgccctg tcttggagat ctactggggg catcctggat |
| 2221 | gtgtatatct tcctggggcc tgagcccaag tctgtggtgc agcagtacct ggatgtggtg |
| 2281 | ggctacccct tcatgccccc ctactggggc ctgggcttcc acctgtgcag gtggggctac |
| 2341 | tcttctactg ctatcaccag gcaggtggtg gagaacatga ccagggctca cttccctctg |
| 2401 | gatgtgcagt ggaatgacct ggactacatg gactctagga gagacttcac ttttaataag |
| 2461 | gatgggttca gggactttcc tgccatggtg caggagctgc atcagggggg caggagatat |
| 2521 | atgatgattg tggaccctgc tatttctagc tctggccctg ctggcagcta taggccctat |
| 2581 | gatgaggggc tgaggagggg ggtgttcatc actaatgaga ctggccagcc cctgattggc |
| 2641 | aaggtgtggc ctggctctac tgccttccct gatttcacca accccactgc cctggcctgg |
| 2701 | tgggaggata tggtggctga gtttcatgac caggtgccct ttgatggcat gtggattgac |
| 2761 | atgaatgagc ccagcaactt tatcaggggc tctgaagatg gctgccccaa caatgagctg |
| 2821 | gagaaccccc cctatgtgcc tggggtggtg gggggcaccc tgcaggctgc caccatctgt |
| 2881 | gccagcagcc accagttcct gagcacccac tataacctgc acaacctgta tggcctgact |
| 2941 | gaggccattg cctctcacag ggccctggtg aaggctaggg ggactaggcc ctttgtgatc |
| 3001 | agcaggtcta cttttgctgg ccatggcagg tatgctgggc actggactgg ggatgtgtgg |
| 3061 | tctagctggg agcagctggc cagctctgtg cctgagatcc tgcagtttaa tctgctgggg |
| 3121 | gtgcccctgg tgggggctga tgtgtgtggc ttcctgggca atacctctga ggagctgtgt |
| 3181 | gtgaggtgga ctcagctggg ggctttctac cccttcatga gaaaccacaa ctctctgctg |
| 3241 | agcctgcccc aggagcccta ttctttttct gagcctgccc agcaggctat gaggaaggcc |
| 3301 | ctgactctga ggtatgccct gctgccccac ctgtataccc tgttccatca ggcccatgtg |
| 3361 | gctggggaga ctgtggccag acctctgttc ctggagttcc ccaaggatag ctctacttgg |
| 3421 | actgtggacc accagctgct gtggggggag gctctgctga tcacccctgt gctgcaggct |
| 3481 | gggaaggctg aggtgactgg ctatttcccc ctgggcacct ggtatgatct gcagactgtg |
| 3541 | cccattgagg ccctgggctc tctgccccct ccccctgctg cccccaggga gcctgccatc |
| 3601 | cactctgagg gccagtgggt gaccctgcct gcccctctgg acactatcaa tgtgcacctg |
| 3661 | agggctggct acatcatccc cctgcagggc cctggcctga ctaccactga gtctaggcag |
| 3721 | cagcccatgg ccctggctgt ggctctgacc aagggggggg aggccagggg ggagctgttc |
| 3781 | tgggatgatg gggagtctct ggaggtgctg gagagggggg cctacaccca ggtgatcttc |
| 3841 | ctggctagga ataacaccat tgtcaatgag ctggtgaggg tgacctctga gggggctggc |
| 3901 | ctgcagctgc agaaggtgac tgtgctgggg gtggctactg ccccccagca ggtgctgagc |
| 3961 | aatggggtgc ctgtgagcaa cttcacctat agccctgaca ccaaggtgct ggacatctgt |
| 4021 | gtgagcctgc tgatggggga gcagttcctg gtgagctggt gctaatttaa atctcgagcc |
| 4081 | gggcggagtg tgttagtctc tccagaggga ggctggttcc ccagggaagc agagcctgtg |
| 4141 | tgcgggcagc agctgtgtgc gggcctgggg gttgttaagt gcaattattt ttaataaaag |
| 4201 | gggcatttgg aaaaaaaaaa aaaaggtagc agtcgacaga tgaattctgc agatctgtgg |
| 4261 | cttctagctg cccgggtggc atccctgtga cccctcccca gtgcctctcc tggccctgga |
| 4321 | agttgccact ccagtgccca ccagccttgt cctaataaaa ttaagttgca tcattttgtc |
| 4381 | tgactaggtg tccttctata atattatggg gtggaggggg gtggtatgga gcaaggggca |
| 4441 | agttgggaag acaacctgta gggcctgcgg ggtctattgg gaaccaagct ggagtgcagt |
| 4501 | ggcacaatct tggctcactg caatctccgc ctcctgggtt caagcgattc tcctgcctca |
| 4561 | gcctcccgag ttgttgggat tccaggcatg catgaccagg ctcagctaat ttttgttttt |
| 4621 | ttggtagaga cggggtttca ccatattggc caggctggtc tccaactcct aatctcaggt |
| 4681 | gatctaccca ccttggcctc ccaaattgct gggattacag gcgtgaacca ctgctccctt |
| 4741 | ccctgtcctt ctgattttaa aaaaaagaga aatgttctgg cacctgcact tgcactgggg |
| 4801 | acagcctatt ttgctagttt gttttgtttc gttttgtttt gatggagagc gtatgttgtt |
| 4861 | taaacgcggc cgcgtagata agtagcatgg cgggttaatc attaactaca aggaacccct |
| 4921 | agtgatggag ttggccactc cctctctgcg cgctcgctcg ctcactgagg ccgggcgacc |
| 4981 | aaaggtcgcc cgacgcccgg gctttgcccg ggcggcctca gtgagcgagc gagcgcgcag |
| 5041 | ttaattaagg cgccctaggc cgacccttag actctgtact cagttctata aacgagccat |
| 5101 | tggatacgag atccgtagat tgataaggga cacggaatat ccccggacgc aatagacacc |
| 5161 | ggtggacagc ttggtatcct gagcacagtc gcgcgtccga atctagctct actttagagg |
| 5221 | ccccggattc tgatggtcgt agaccgcaga accgattggg gggatgagat ctactagtta |
| 5281 | tcagcacaca attgcccatt atacgcgcgt ataatggact attgtgtgct gatataggga |
| 5341 | taacagggta attctagagc tagcatatgg atccatcgat ttgatgcggt attttctcct |
| 5401 | tacgcatctg tgcggtattt cacaccgcat acgtcaaagc aaccatagta cgcgccctgt |
| 5461 | agcggcgcat taagcgcggc gggtgtggtg gttacgcgca gcgtgaccgc tacacttgcc |
| 5521 | agcgccctag cgcccgctcc tttcgctttc ttcccttcct ttctcgccac gttcgccggc |
| 5581 | tttccccgtc aagctctaaa tcgggggctc cctttagggt tccgatttag tgctttacgg |
| 5641 | cacctcgacc ccaaaaaact tgatttgggt gatggttcac gtagtgggcc atcgccctga |
| 5701 | tagacggttt ttcgcccttt gacgttggag tccacgttct ttaatagtgg actcttgttc |
| 5761 | caaactggaa caacactcaa ctctatctcg ggctattctt ttgatttata agggattttg |
| 5821 | ccgatttcgg tctattggtt aaaaaatgag ctgatttaac aaaaatttaa cgcgaatttt |
| 5881 | aacaaaatat taacgtttac aattttatgg tgcactctca gtacaatctg ctctgatgcc |
| 5941 | gcatagttaa gccagccccg acacccgcca acacccgctg acgcgccctg acgggcttgt |
| 6001 | ctgctcccgg catccgctta cagacaagct gtgaccgtct ccgggagctg catgtgtcag |
| 6061 | aggttttcac cgtcatcacc gaaacgcgcg agacgaaagg gcctcgtgat acgcctattt |
| 6121 | ttataggtta atgtcatgat aataatggtt tcttagacgt caggtggcac ttttcgggga |
| 6181 | aatgtgcgcg gaacccctat ttgtttattt ttctaaatac attcaaatat gtatccgctc |
| 6241 | atgagacaat aaccctgata aatgcttcaa taatattgaa aaaggaagag tatgagccat |
| 6301 | attcaacggg aaacgtcgag gccgcgatta aattccaaca tggatgctga tttatatggg |
| 6361 | tataaatggg ctcgcgataa tgtcgggcaa tcaggtgcga caatctatcg cttgtatggg |
| 6421 | aagcccgatg cgccagagtt gtttctgaaa catggcaaag gtagcgttgc caatgatgtt |
| 6481 | acagatgaga tggtcagact aaactggctg acggaattta tgcctcttcc gaccatcaag |
| 6541 | cattttatcc gtactcctga tgatgcatgg ttactcacca ctgcgatccc cggaaaaaca |
| 6601 | gcattccagg tattagaaga atatcctgat tcaggtgaaa atattgttga tgcgctggca |
| 6661 | gtgttcctgc gccggttgca ttcgattcct gtttgtaatt gtccttttaa cagcgatcgc |
| 6721 | gtatttcgtc tcgctcaggc gcaatcacga atgaataacg gtttggttga tgcgagtgat |
| 6781 | tttgatgacg agcgtaatgg ctggcctgtt gaacaagtct ggaaagaaat gcataaactt |
| 6841 | ttgccattct caccggattc agtcgtcact catggtgatt tctcacttga taaccttatt |
| 6901 | tttgacgagg ggaaattaat aggttgtatt gatgttggac gagtcggaat cgcagaccga |
| 6961 | taccaggatc ttgccatcct atggaactgc ctcggtgagt tttctccttc attacagaaa |
| 7021 | cggctttttc aaaaatatgg tattgataat cctgatatga ataaattgca gtttcatttg |
| 7081 | atgctcgatg agtttttcta agcgtataat ggtctagagc tagcatatgg atccatcgat |
| 7141 | tccattatac gcctgtcaga ccaagtttac tcatatatac tttagattga tttaaaactt |
| 7201 | catttttaat ttaaaaggat ctaggtgaag atcctttttg ataatctcat gaccaaaatc |
| 7261 | ccttaacgtg agttttcgtt ccactgagcg tcagaccccg tagaaaagat caaaggatct |
| 7321 | tcttgagatc ctttttttct gcgcgtaatc tgctgcttgc aaacaaaaaa accaccgcta |
| 7381 | ccagcggtgg tttgtttgcc ggatcaagag ctaccaactc tttttccgaa ggtaactggc |
| 7441 | ttcagcagag cgcagatacc aaatactgtt cttctagtgt agccgtagtt aggccaccac |
| 7501 | ttcaagaact ctgtagcacc gcctacatac ctcgctctgc taatcctgtt accagtggct |
| 7561 | gctgccagtg gcgataagtc gtgtcttacc gggttggact caagacgata gttaccggat |
| 7621 | aaggcgcagc ggtcgggctg aacggggggt tcgtgcacac agcccagctt ggagcgaacg |
| 7681 | acctacaccg aactgagata cctacagcgt gagctatgag aaagcgccac gcttcccgaa |
| 7741 | gggagaaagg cggacaggta tccggtaagc ggcagggtcg gaacaggaga gcgcacgagg |
| 7801 | gagcttccag ggggaaacgc ctggtatctt tatagtcctg tcgggtttcg ccacctctga |
| 7861 | cttgagcgtc gatttttgtg atgctcgtca ggggggcgga gcctatggaa aaacgccagc |
| 7921 | aacgcggcct ttttacggtt cctggccttt tgctggcctt ttgctcacat gt |

| pP080_seq100-201Ig_3-56 deletion (SEQ ID NO: 485) | |
|---|---|
| (SEQ ID NO: 485) | |
| 1 | tctagagcta gcatatggat ccatcgattt agggataaca gggtaattat cagcacacaa |
| 61 | ttgcccatta tacgcgcgta taatggacta ttgtgtgctg atatctgtac acttaagggc |
| 121 | tagatcttag cttacgtcac tagagggtcc acgtttagtt tttaagatcc attgatctcc |
| 181 | taaacgctgc aagattcgca acctggtata cttagcctag gcgctaggtc ctagtgcagc |
| 241 | gggacttttt ttctaaagtc gttgagagga ggagtcgtca gaccagatag ctttgatgtc |
| 301 | ctgatcggaa ggatcgttgg cccccctgca ggcagctgtt aattaactgc gcgctcgctc |
| 361 | gctcactgag gccgcccggg caaagcccgg gcgtcgggcg acctttggtc gcccggcctc |
| 421 | agtgagcgag cgagcgcgca gagagggagt ggccaactcc atcactaggg gttccttgta |
| 481 | gttaatgatt aacccgccat gctacttatc tacggcgcgc cacgcgttct aggagttaat |
| 541 | ttttaaaaag cagtcaaaag tccaagtgcc cttgcgagca tttactctct ctgtttgctc |
| 601 | tggttaataa tctcaggagc acaaacattc cttactagtt ctaggagtta atttttaaaa |
| 661 | agcagtcaaa agtccaagtg cccttgcgag catttactct ctctgtttgc tctggttaat |
| 721 | aatctcagga gcacaaacat tccttactag ttctagagcg gccgccagtg tgctggaatt |
| 781 | cggctttttt agggctggaa gctacctttg acatcatttc ctctgcgaat gcatgtataa |
| 841 | tttctacaga acctattaga aaggatcacc cagcctctgc ttttgtacaa ctttccctta |
| 901 | aaaaactgcc aatcccactg ctgtttggcc caatagtgag aactttttcc tgctgcctct |
| 961 | tggtgctttt gcctatggcc cctattctgc ctgctgaaga cactcttgcc agcatggact |
| 1021 | taaacccctc cagctctgac aatcctcttt ctcttttgtt ttacatgaag ggtctggcag |
| 1081 | ccaaagcaat cactcaaagt tcaaacctta tcattttttg ctttgttcct cttggccttg |
| 1141 | gttttgtaca tcagctttga aaataccatc ccagggttaa tgctggggtt aatttataac |
| 1201 | tgagagtgct ctagttctgc aatacaggac atgctataaa aatggaaaga tgttgctttc |
| 1261 | tgagagatca ggtggattct tgggcatttg cttaagcttg gtaccgccac catgggggag |
| 1321 | tttgggctga gctgggtctt tctggtggcc ctgctgaagg gagtccagtg tgagcagcag |
| 1381 | ggggccagca ggccaggccc cagggatgcc caggcccacc ctggcagacc cagggctgtg |
| 1441 | cccacccagt gtgatgtgcc tcccaacagc agatttgact gtgcccctga taaggccatt |
| 1501 | actcaggaac agtgtgaggc caggggctgc tgctatatcc ctgccaagca gggcctgcag |
| 1561 | ggggcccaga tggggcagcc ctggtgcttc ttccccccca gctacccctc ttataagctg |
| 1621 | gagaatctga gcagctctga aatgggctac actgccactc tgaccaggac tacccccacc |
| 1681 | ttcttcccca aggatattct gactctgagg ctggatgtga tgatggagac tgagaacagg |
| 1741 | ctgcacttca ccatcaagga ccctgccaac aggaggtatg aggtgcccct ggaaactccc |
| 1801 | agggtgcact ctagggcccc cagccccctg tattctgtgg agttctctga ggagcctttt |
| 1861 | ggggtcattg tccacaggca gctggatggc agggtgctgc tgaatactac tgtggcccct |
| 1921 | ctgttctttg ctgaccagtt cctgcagctg agcaccagcc tgccttctca gtacattact |
| 1981 | ggcctggctg agcatctgag ccccctgatg ctgagcacct cttggaccag aatcaccctg |
| 2041 | tggaacaggg acctggctcc cactcctggg gccaacctgt atggcagcca ccccttctac |
| 2101 | ctggccctgg aggatggggg ctctgcccat ggggtgttcc tgctgaacag caatgccatg |
| 2161 | gatgtggtgc tgcagccctc tcctgccctg tcttggagat ctactggggg catcctggat |
| 2221 | gtgtatatct tcctggggcc tgagcccaag tctgtggtgc agcagtacct ggatgtggtg |
| 2281 | ggctacccct tcatgccccc ctactggggc ctgggcttcc acctgtgcag gtggggctac |
| 2341 | tcttctactg ctatcaccag gcaggtggtg gagaacatga ccagggctca cttccctctg |
| 2401 | gatgtgcagt ggaatgacct ggactacatg gactctagga gagacttcac ttttaataag |
| 2461 | gatgggttca gggactttcc tgccatggtg caggagctgc atcagggggg caggagatat |
| 2521 | atgatgattg tggaccctgc tatttctagc tctggccctg ctggcagcta taggccctat |
| 2581 | gatgaggggc tgaggagggg ggtgttcatc actaatgaga ctggccagcc cctgattggc |
| 2641 | aaggtgtggc ctggctctac tgccttccct gatttcacca accccactgc cctggcctgg |
| 2701 | tgggaggata tggtggctga gtttcatgac caggtgccct ttgatggcat gtggattgac |
| 2761 | atgaatgagc ccagcaactt tatcaggggc tctgaagatg gctgccccaa caatgagctg |
| 2821 | gagaaccccc cctatgtgcc tggggtggtg gggggcaccc tgcaggctgc caccatctgt |
| 2881 | gccagcagcc accagttcct gagcacccac tataacctgc acaacctgta tggcctgact |
| 2941 | gaggccattg cctctcacag ggccctggtg aaggctaggg ggactaggcc ctttgtgatc |
| 3001 | agcaggtcta cttttgctgg ccatggcagg tatgctgggc actggactgg ggatgtgtgg |
| 3061 | tctagctggg agcagctggc cagctctgtg cctgagatcc tgcagtttaa tctgctgggg |
| 3121 | gtgcccctgg tgggggctga tgtgtgtggc ttcctgggca atacctctga ggagctgtgt |
| 3181 | gtgaggtgga ctcagctggg ggctttctac cccttcatga gaaaccacaa ctctctgctg |
| 3241 | agcctgcccc aggagcccta ttctttttct gagcctgccc agcaggctat gaggaaggcc |
| 3301 | ctgactctga ggtatgccct gctgccccac ctgtataccc tgttccatca ggcccatgtg |
| 3361 | gctggggaga ctgtggccag acctctgttc ctggagttcc ccaaggatag ctctacttgg |
| 3421 | actgtggacc accagctgct gtggggggag gctctgctga tcacccctgt gctgcaggct |
| 3481 | gggaaggctg aggtgactgg ctatttcccc ctgggcacct ggtatgatct gcagactgtg |
| 3541 | cccattgagg ccctgggctc tctgccccct ccccctgctg cccccaggga gcctgccatc |
| 3601 | cactctgagg gccagtgggt gaccctgcct gcccctctgg acactatcaa tgtgcacctg |
| 3661 | agggctggct acatcatccc cctgcagggc cctggcctga ctaccactga gtctaggcag |
| 3721 | cagcccatgg ccctggctgt ggctctgacc aagggggggg aggccagggg ggagctgttc |
| 3781 | tgggatgatg gggagtctct ggaggtgctg gagagggggg cctacaccca ggtgatcttc |
| 3841 | ctggctagga ataacaccat tgtcaatgag ctggtgaggg tgacctctga gggggctggc |
| 3901 | ctgcagctgc agaaggtgac tgtgctgggg gtggctactg ccccccagca ggtgctgagc |
| 3961 | aatggggtgc ctgtgagcaa cttcacctat agccctgaca ccaaggtgct ggacatctgt |
| 4021 | gtgagcctgc tgatggggga gcagttcctg gtgagctggt gctaatttaa atctcgagcc |
| 4081 | gggcggagtg tgttagtctc tccagaggga ggctggttcc ccagggaagc agagcctgtg |
| 4141 | tgcgggcagc agctgtgtgc gggcctgggg gttgttaagt gcaattattt ttaataaaag |
| 4201 | gggcatttgg aaaaaaaaaa aaaaggtagc agtcgacaga tgaattctgc agatctgtgg |
| 4261 | cttctagctg cccgggtggc atccctgtga cccctcccca gtgcctctcc tggccctgga |
| 4321 | agttgccact ccagtgccca ccagccttgt cctaataaaa ttaagttgca tcattttgtc |
| 4381 | tgactaggtg tccttctata atattatggg gtggaggggg gtggtatgga gcaaggggca |
| 4441 | agttgggaag acaacctgta gggcctgcgg ggtctattgg gaaccaagct ggagtgcagt |
| 4501 | ggcacaatct tggctcactg caatctccgc ctcctgggtt caagcgattc tcctgcctca |
| 4561 | gcctcccgag ttgttgggat tccaggcatg catgaccagg ctcagctaat ttttgttttt |
| 4621 | ttggtagaga cggggtttca ccatattggc caggctggtc tccaactcct aatctcaggt |
| 4681 | gatctaccca ccttggcctc ccaaattgct gggattacag gcgtgaacca ctgctccctt |
| 4741 | ccctgtcctt ctgattttaa aaaaaagaga aatgttctgg cacctgcact tgcactgggg |
| 4801 | acagcctatt ttgctagttt gttttgtttc gttttgtttt gatggagagc gtatgttgtt |
| 4861 | taaacgcggc cgcgtagata agtagcatgg cgggttaatc attaactaca aggaacccct |
| 4921 | agtgatggag ttggccactc cctctctgcg cgctcgctcg ctcactgagg ccgggcgacc |
| 4981 | aaaggtcgcc cgacgcccgg gctttgcccg ggcggcctca gtgagcgagc gagcgcgcag |
| 5041 | ttaattaagg cgccctaggc cgacccttag actctgtact cagttctata aacgagccat |
| 5101 | tggatacgag atccgtagat tgataaggga cacggaatat ccccggacgc aatagacacc |
| 5161 | ggtggacagc ttggtatcct gagcacagtc gcgcgtccga atctagctct actttagagg |
| 5221 | ccccggattc tgatggtcgt agaccgcaga accgattggg gggatgagat ctactagtta |
| 5281 | tcagcacaca attgcccatt atacgcgcgt ataatggact attgtgtgct gatataggga |
| 5341 | taacagggta attctagagc tagcatatgg atccatcgat ttgatgcggt attttctcct |
| 5401 | tacgcatctg tgcggtattt cacaccgcat acgtcaaagc aaccatagta cgcgccctgt |
| 5461 | agcggcgcat taagcgcggc gggtgtggtg gttacgcgca gcgtgaccgc tacacttgcc |
| 5521 | agcgccctag cgcccgctcc tttcgctttc ttcccttcct ttctcgccac gttcgccggc |
| 5581 | tttccccgtc aagctctaaa tcgggggctc cctttagggt tccgatttag tgctttacgg |
| 5641 | cacctcgacc ccaaaaaact tgatttgggt gatggttcac gtagtgggcc atcgccctga |
| 5701 | tagacggttt ttcgcccttt gacgttggag tccacgttct ttaatagtgg actcttgttc |
| 5761 | caaactggaa caacactcaa ctctatctcg ggctattctt ttgatttata agggattttg |
| 5821 | ccgatttcgg tctattggtt aaaaaatgag ctgatttaac aaaaatttaa cgcgaatttt |
| 5881 | aacaaaatat taacgtttac aattttatgg tgcactctca gtacaatctg ctctgatgcc |
| 5941 | gcatagttaa gccagccccg acacccgcca acacccgctg acgcgccctg acgggcttgt |
| 6001 | ctgctcccgg catccgctta cagacaagct gtgaccgtct ccgggagctg catgtgtcag |
| 6061 | aggttttcac cgtcatcacc gaaacgcgcg agacgaaagg gcctcgtgat acgcctattt |
| 6121 | ttataggtta atgtcatgat aataatggtt tcttagacgt caggtggcac ttttcgggga |
| 6181 | aatgtgcgcg gaacccctat ttgtttattt ttctaaatac attcaaatat gtatccgctc |
| 6241 | atgagacaat aaccctgata aatgcttcaa taatattgaa aaaggaagag tatgagccat |
| 6301 | attcaacggg aaacgtcgag gccgcgatta aattccaaca tggatgctga tttatatggg |
| 6361 | tataaatggg ctcgcgataa tgtcgggcaa tcaggtgcga caatctatcg cttgtatggg |
| 6421 | aagcccgatg cgccagagtt gtttctgaaa catggcaaag gtagcgttgc caatgatgtt |
| 6481 | acagatgaga tggtcagact aaactggctg acggaattta tgcctcttcc gaccatcaag |
| 6541 | cattttatcc gtactcctga tgatgcatgg ttactcacca ctgcgatccc cggaaaaaca |
| 6601 | gcattccagg tattagaaga atatcctgat tcaggtgaaa atattgttga tgcgctggca |
| 6661 | gtgttcctgc gccggttgca ttcgattcct gtttgtaatt gtccttttaa cagcgatcgc |
| 6721 | gtatttcgtc tcgctcaggc gcaatcacga atgaataacg gtttggttga tgcgagtgat |
| 6781 | tttgatgacg agcgtaatgg ctggcctgtt gaacaagtct ggaaagaaat gcataaactt |
| 6841 | ttgccattct caccggattc agtcgtcact catggtgatt tctcacttga taaccttatt |
| 6901 | tttgacgagg ggaaattaat aggttgtatt gatgttggac gagtcggaat cgcagaccga |
| 6961 | taccaggatc ttgccatcct atggaactgc ctcggtgagt tttctccttc attacagaaa |
| 7021 | cggctttttc aaaaatatgg tattgataat cctgatatga ataaattgca gtttcatttg |
| 7081 | atgctcgatg agtttttcta agcgtataat ggtctagagc tagcatatgg atccatcgat |
| 7141 | tccattatac gcctgtcaga ccaagtttac tcatatatac tttagattga tttaaaactt |
| 7201 | catttttaat ttaaaaggat ctaggtgaag atcctttttg ataatctcat gaccaaaatc |
| 7261 | ccttaacgtg agttttcgtt ccactgagcg tcagaccccg tagaaaagat caaaggatct |
| 7321 | tcttgagatc ctttttttct gcgcgtaatc tgctgcttgc aaacaaaaaa accaccgcta |
| 7381 | ccagcggtgg tttgtttgcc ggatcaagag ctaccaactc tttttccgaa ggtaactggc |
| 7441 | ttcagcagag cgcagatacc aaatactgtt cttctagtgt agccgtagtt aggccaccac |
| 7501 | ttcaagaact ctgtagcacc gcctacatac ctcgctctgc taatcctgtt accagtggct |
| 7561 | gctgccagtg gcgataagtc gtgtcttacc gggttggact caagacgata gttaccggat |
| 7621 | aaggcgcagc ggtcgggctg aacggggggt tcgtgcacac agcccagctt ggagcgaacg |
| 7681 | acctacaccg aactgagata cctacagcgt gagctatgag aaagcgccac gcttcccgaa |
| 7741 | gggagaaagg cggacaggta tccggtaagc ggcagggtcg gaacaggaga gcgcacgagg |
| 7801 | gagcttccag ggggaaacgc ctggtatctt tatagtcctg tcgggtttcg ccacctctga |
| 7861 | cttgagcgtc gatttttgtg atgctcgtca ggggggcgga gcctatggaa aaacgccagc |
| 7921 | aacgcggcct ttttacggtt cctggccttt tgctggcctt ttgctcacat gt |

| pP081_seq3-IL2wt_3-56 deletion (SEQ ID NO: 486) | |
|---|---|
| (SEQ ID NO: 486) | |
| 1 | tctagagcta gcatatggat ccatcgattt agggataaca gggtaattat cagcacacaa |
| 61 | ttgcccatta tacgcgcgta taatggacta ttgtgtgctg atatctgtac acttaagggc |
| 121 | tagatcttag cttacgtcac tagagggtcc acgtttagtt tttaagatcc attgatctcc |
| 181 | taaacgctgc aagattcgca acctggtata cttagcctag gcgctaggtc ctagtgcagc |
| 241 | gggacttttt ttctaaagtc gttgagagga ggagtcgtca gaccagatag ctttgatgtc |
| 301 | ctgatcggaa ggatcgttgg cccccctgca ggcagctgtt aattaactgc gcgctcgctc |
| 361 | gctcactgag gccgcccggg caaagcccgg gcgtcgggcg acctttggtc gcccggcctc |
| 421 | agtgagcgag cgagcgcgca gagagggagt ggccaactcc atcactaggg gttccttgta |
| 481 | gttaatgatt aacccgccat gctacttatc tacggcgcgc cacgcgttct aggagttaat |
| 541 | ttttaaaaag cagtcaaaag tccaagtgcc cttgcgagca tttactctct ctgtttgctc |
| 601 | tggttaataa tctcaggagc acaaacattc cttactagtt ctaggagtta atttttaaaa |
| 661 | agcagtcaaa agtccaagtg cccttgcgag catttactct ctctgtttgc tctggttaat |
| 721 | aatctcagga gcacaaacat tccttactag ttctagagcg gccgccagtg tgctggaatt |
| 781 | cggctttttt agggctggaa gctacctttg acatcatttc ctctgcgaat gcatgtataa |
| 841 | tttctacaga acctattaga aaggatcacc cagcctctgc ttttgtacaa ctttccctta |
| 901 | aaaaactgcc aatcccactg ctgtttggcc caatagtgag aactttttcc tgctgcctct |
| 961 | tggtgctttt gcctatggcc cctattctgc ctgctgaaga cactcttgcc agcatggact |
| 1021 | taaacccctc cagctctgac aatcctcttt ctcttttgtt ttacatgaag ggtctggcag |
| 1081 | ccaaagcaat cactcaaagt tcaaacctta tcattttttg ctttgttcct cttggccttg |
| 1141 | gttttgtaca tcagctttga aaataccatc ccagggttaa tgctggggtt aatttataac |
| 1201 | tgagagtgct ctagttctgc aatacaggac atgctataaa aatggaaaga tgttgctttc |
| 1261 | tgagagatca ggtggattct tgggcatttg cttaagcttg gtaccgccac catggggtac |
| 1321 | agaatgcagc tgctgtcctg cattgcactg tcactggcac tggttaccaa ctcacagcag |
| 1381 | ggagcttcta gacctggacc tagagatgct caggctcatc ctggaagacc tagagctgtg |
| 1441 | cctacacagt gtgatgtgcc tcctaattct agatttgatt gtgctcctga taaggctata |
| 1501 | acacaggagc agtgtgaggc tagaggatgt tgttatattc ctgctaagca gggactgcag |
| 1561 | ggagctcaga tgggacagcc ttggtgtttt tttcctcctt cttatccttc ttataagctg |
| 1621 | gagaatctgt cttcttctga gatgggatat acagctacac tgacaagaac aacacctaca |
| 1681 | ttttttccta aggatattct gacactgaga ctggatgtga tgatggagac agagaataga |
| 1741 | ctgcatttta caattaagga tcctgctaat agaagatatg aggtgcctct agagacacct |
| 1801 | agagtgcatt ctagagctcc ttctcctctg tattctgtgg agttttctga ggagcctttt |
| 1861 | ggagtgattg tgcatagaca gctggatgga agagtgctgc tgaatacaac agtggctcct |
| 1921 | ctgttttttg ctgatcagtt tctgcagctg tctacatctc tgccttctca gtatattaca |
| 1981 | ggactggctg agcatctgtc tcctctgatg ctgtctacat cttggacaag aattacactg |
| 2041 | tggaatagag atctggctcc tacacctgga gctaatctgt atggatctca tcctttttat |
| 2101 | ctggctctgg aggatggagg atctgctcat ggagtgtttc tgctgaattc taatgctatg |
| 2161 | gatgtagtgc tgcagccttc tcctgctctg tcttggagat ctacaggagg aattctggat |
| 2221 | gtgtatattt ttctaggacc tgagcctaag tctgtggtgc agcagtatct ggatgtagtg |
| 2281 | ggatatcctt ttatgcctcc ttattgggga ctgggatttc atctgtgtag atggggatat |
| 2341 | tcttctacag ctattacaag acaggtggtt gagaatatga caagagctca ttttcctctg |
| 2401 | gatgtgcagt ggaatgatct agattatatg gattctagaa gagattttac atttaataag |
| 2461 | gatggattta gagattttcc tgctatggtg caggagctgc atcagggagg aagaagatat |
| 2521 | atgatgattg tggatcctgc tatttcttct tctggacctg ctggatctta tagaccttat |
| 2581 | gatgagggac tgagaagagg agtgtttatt acaaatgaga caggacagcc tctgattgga |
| 2641 | aaggtgtggc ctggatctac agcttttcct gattttacaa atcctacagc tctggcttgg |
| 2701 | tgggaggata tggtggctga gtttcatgat caggtgcctt ttgatggaat gtggattgat |
| 2761 | atgaatgagc cttctaattt tataagagga tctgaggatg gatgtcctaa taatgagcta |
| 2821 | gagaatcctc cttatgtgcc tggagtagtg ggaggaacac tgcaggctgc tacaatttgt |
| 2881 | gcttcttctc atcagtttct gtctacacat tataatctgc ataatctgta tggactgaca |
| 2941 | gaggctattg cttctcatag agctctggtg aaggctagag gaacaagacc ttttgtgatt |
| 3001 | tctagatcta catttgctgg acatggaaga tatgctggac attggacagg agatgtatgg |
| 3061 | tcttcttggg agcagctagc ttcttctgtg cctgagattc tgcagtttaa tctgctagga |
| 3121 | gtgcctctgg tgggagctga tgtgtgtgga tttctgggaa atacatctga ggagctgtgt |
| 3181 | gtgagatgga cacagctggg agctttttat ccttttatga gaaatcataa ttctctgctg |
| 3241 | tctctgcctc aggagcctta ttctttttct gagcctgctc agcaggctat gagaaaggct |
| 3301 | ctgacactga gatatgctct gctgcctcat ctgtatacac tgtttcatca ggctcatgtg |
| 3361 | gctggagaga cagtagctag acctctgttt ctggagtttc ctaaggattc ttctacatgg |
| 3421 | acagttgatc atcagctgct gtggggagag gctctgctga ttacacctgt gctgcaggct |
| 3481 | ggaaaggctg aggtgacagg atattttcct ctgggaacat ggtatgatct gcagacagtg |
| 3541 | cctatagagg ctctgggatc tctgcctcct cctcctgctg ctcctagaga gcctgctatt |
| 3601 | cattctgagg gacagtgggt gacactgcct gctcctctgg atacaattaa tgtgcatctg |
| 3661 | agagctggat atattattcc tctgcaggga cctggactga caacaacaga gtctagacag |
| 3721 | cagcctatgg ctctggctgt ggctctgaca aagggaggag aggctagagg agagctgttt |
| 3781 | tgggatgatg gagagtctct ggaggtgctg gagagaggag cttatacaca ggtgattttt |
| 3841 | ctggctagaa ataatacaat tgtgaatgag ctggtgagag tgacatctga gggagctgga |
| 3901 | ctgcagctgc agaaggtgac agtgctggga gtggctacag ctcctcagca ggtgctgtct |
| 3961 | aatggagtgc ctgtgtctaa ttttacatat tctcctgata caaaggtgct ggatatttgt |
| 4021 | gtgtctctgc tgatgggaga gcagtttctg gtgtcttggt gttaatgatt taaatctcga |
| 4081 | gccgggcgga gtgtgttagt ctctccagag ggaggctggt tccccaggga agcagagcct |
| 4141 | gtgtgcgggc agcagctgtg tgcgggcctg ggggttgtta agtgcaatta tttttaataa |
| 4201 | aaggggcatt tggaaaaaaa aaaaaaaggt agcagtcgac agatgaattc tgcagatctg |
| 4261 | tggcttctag ctgcccgggt ggcatccctg tgacccctcc ccagtgcctc tcctggccct |
| 4321 | ggaagttgcc actccagtgc ccaccagcct tgtcctaata aaattaagtt gcatcatttt |
| 4381 | gtctgactag gtgtccttct ataatattat ggggtggagg ggggtggtat ggagcaaggg |
| 4441 | gcaagttggg aagacaacct gtagggcctg cggggtctat tgggaaccaa gctggagtgc |
| 4501 | agtggcacaa tcttggctca ctgcaatctc cgcctcctgg gttcaagcga ttctcctgcc |
| 4561 | tcagcctccc gagttgttgg gattccaggc atgcatgacc aggctcagct aatttttgtt |
| 4621 | tttttggtag agacggggtt tcaccatatt ggccaggctg gtctccaact cctaatctca |
| 4681 | ggtgatctac ccaccttggc ctcccaaatt gctgggatta caggcgtgaa ccactgctcc |
| 4741 | cttccctgtc cttctgattt taaaaaaaag agaaatgttc tggcacctgc acttgcactg |
| 4801 | gggacagcct attttgctag tttgttttgt ttcgttttgt tttgatggag agcgtatgtt |
| 4861 | gtttaaacgc ggccgcgtag ataagtagca tggcgggtta atcattaact acaaggaacc |
| 4921 | cctagtgatg gagttggcca ctccctctct gcgcgctcgc tcgctcactg aggccgggcg |
| 4981 | accaaaggtc gcccgacgcc cgggctttgc ccgggcggcc tcagtgagcg agcgagcgcg |
| 5041 | cagttaatta aggcgcccta ggccgaccct tagactctgt actcagttct ataaacgagc |
| 5101 | cattggatac gagatccgta gattgataag ggacacggaa tatccccgga cgcaatagac |
| 5161 | accggtggac agcttggtat cctgagcaca gtcgcgcgtc cgaatctagc tctactttag |
| 5221 | aggccccgga ttctgatggt cgtagaccgc agaaccgatt ggggggatga gatctactag |
| 5281 | ttatcagcac acaattgccc attatacgcg cgtataatgg actattgtgt gctgatatag |
| 5341 | ggataacagg gtaattctag agctagcata tggatccatc gatttgatgc ggtattttct |
| 5401 | ccttacgcat ctgtgcggta tttcacaccg catacgtcaa agcaaccata gtacgcgccc |
| 5461 | tgtagcggcg cattaagcgc ggcgggtgtg gtggttacgc gcagcgtgac cgctacactt |
| 5521 | gccagcgccc tagcgcccgc tcctttcgct ttcttccctt cctttctcgc cacgttcgcc |
| 5581 | ggctttcccc gtcaagctct aaatcggggg ctccctttag ggttccgatt tagtgcttta |
| 5641 | cggcacctcg accccaaaaa acttgatttg ggtgatggtt cacgtagtgg gccatcgccc |
| 5701 | tgatagacgg tttttcgccc tttgacgttg gagtccacgt tctttaatag tggactcttg |
| 5761 | ttccaaactg gaacaacact caactctatc tcgggctatt cttttgattt ataagggatt |
| 5821 | ttgccgattt cggtctattg gttaaaaaat gagctgattt aacaaaaatt taacgcgaat |
| 5881 | tttaacaaaa tattaacgtt tacaatttta tggtgcactc tcagtacaat ctgctctgat |
| 5941 | gccgcatagt taagccagcc ccgacacccg ccaacacccg ctgacgcgcc ctgacgggct |
| 6001 | tgtctgctcc cggcatccgc ttacagacaa gctgtgaccg tctccgggag ctgcatgtgt |
| 6061 | cagaggtttt caccgtcatc accgaaacgc gcgagacgaa agggcctcgt gatacgccta |
| 6121 | tttttatagg ttaatgtcat gataataatg gtttcttaga cgtcaggtgg cacttttcgg |
| 6181 | ggaaatgtgc gcggaacccc tatttgttta tttttctaaa tacattcaaa tatgtatccg |
| 6241 | ctcatgagac aataaccctg ataaatgctt caataatatt gaaaaaggaa gagtatgagc |
| 6301 | catattcaac gggaaacgtc gaggccgcga ttaaattcca acatggatgc tgatttatat |
| 6361 | gggtataaat gggctcgcga taatgtcggg caatcaggtg cgacaatcta tcgcttgtat |
| 6421 | gggaagcccg atgcgccaga gttgtttctg aaacatggca aaggtagcgt tgccaatgat |
| 6481 | gttacagatg agatggtcag actaaactgg ctgacggaat ttatgcctct tccgaccatc |
| 6541 | aagcatttta tccgtactcc tgatgatgca tggttactca ccactgcgat ccccggaaaa |
| 6601 | acagcattcc aggtattaga agaatatcct gattcaggtg aaaatattgt tgatgcgctg |
| 6661 | gcagtgttcc tgcgccggtt gcattcgatt cctgtttgta attgtccttt taacagcgat |
| 6721 | cgcgtatttc gtctcgctca ggcgcaatca cgaatgaata acggtttggt tgatgcgagt |
| 6781 | gattttgatg acgagcgtaa tggctggcct gttgaacaag tctggaaaga aatgcataaa |
| 6841 | cttttgccat tctcaccgga ttcagtcgtc actcatggtg atttctcact tgataacctt |
| 6901 | atttttgacg aggggaaatt aataggttgt attgatgttg gacgagtcgg aatcgcagac |
| 6961 | cgataccagg atcttgccat cctatggaac tgcctcggtg agttttctcc ttcattacag |
| 7021 | aaacggcttt ttcaaaaata tggtattgat aatcctgata tgaataaatt gcagtttcat |
| 7081 | ttgatgctcg atgagttttt ctaagcgtat aatggtctag agctagcata tggatccatc |
| 7141 | gattccatta tacgcctgtc agaccaagtt tactcatata tactttagat tgatttaaaa |
| 7201 | cttcattttt aatttaaaag gatctaggtg aagatccttt ttgataatct catgaccaaa |
| 7261 | atcccttaac gtgagttttc gttccactga gcgtcagacc ccgtagaaaa gatcaaagga |
| 7321 | tcttcttgag atcctttttt tctgcgcgta atctgctgct tgcaaacaaa aaaaccaccg |
| 7381 | ctaccagcgg tggtttgttt gccggatcaa gagctaccaa ctctttttcc gaaggtaact |
| 7441 | ggcttcagca gagcgcagat accaaatact gttcttctag tgtagccgta gttaggccac |
| 7501 | cacttcaaga actctgtagc accgcctaca tacctcgctc tgctaatcct gttaccagtg |
| 7561 | gctgctgcca gtggcgataa gtcgtgtctt accgggttgg actcaagacg atagttaccg |
| 7621 | gataaggcgc agcggtcggg ctgaacgggg ggttcgtgca cacagcccag cttggagcga |
| 7681 | acgacctaca ccgaactgag atacctacag cgtgagctat gagaaagcgc cacgcttccc |
| 7741 | gaagggagaa aggcggacag gtatccggta agcggcaggg tcggaacagg agagcgcacg |
| 7801 | agggagcttc cagggggaaa cgcctggtat ctttatagtc ctgtcgggtt tcgccacctc |
| 7861 | tgacttgagc gtcgattttt gtgatgctcg tcaggggggc ggagcctatg gaaaaacgcc |
| 7921 | agcaacgcgg cctttttacg gttcctggcc ttttgctggc cttttgctca catgt |

| pP082_seq3-201Ig_3-56 deletion (SEQ ID NO: 487) | |
|---|---|
| (SEQ ID NO: 487) | |
| 1 | tctagagcta gcatatggat ccatcgattt agggataaca gggtaattat cagcacacaa |
| 61 | ttgcccatta tacgcgcgta taatggacta ttgtgtgctg atatctgtac acttaagggc |
| 121 | tagatcttag cttacgtcac tagagggtcc acgtttagtt tttaagatcc attgatctcc |
| 181 | taaacgctgc aagattcgca acctggtata cttagcctag gcgctaggtc ctagtgcagc |
| 241 | gggacttttt ttctaaagtc gttgagagga ggagtcgtca gaccagatag ctttgatgtc |
| 301 | ctgatcggaa ggatcgttgg cccccctgca ggcagctgtt aattaactgc gcgctcgctc |
| 361 | gctcactgag gccgcccggg caaagcccgg gcgtcgggcg acctttggtc gcccggcctc |
| 421 | agtgagcgag cgagcgcgca gagagggagt ggccaactcc atcactaggg gttccttgta |
| 481 | gttaatgatt aacccgccat gctacttatc tacggcgcgc cacgcgttct aggagttaat |
| 541 | ttttaaaaag cagtcaaaag tccaagtgcc cttgcgagca tttactctct ctgtttgctc |
| 601 | tggttaataa tctcaggagc acaaacattc cttactagtt ctaggagtta atttttaaaa |
| 661 | agcagtcaaa agtccaagtg cccttgcgag catttactct ctctgtttgc tctggttaat |
| 721 | aatctcagga gcacaaacat tccttactag ttctagagcg gccgccagtg tgctggaatt |
| 781 | cggctttttt agggctggaa gctacctttg acatcatttc ctctgcgaat gcatgtataa |
| 841 | tttctacaga acctattaga aaggatcacc cagcctctgc ttttgtacaa ctttccctta |
| 901 | aaaaactgcc aatcccactg ctgtttggcc caatagtgag aactttttcc tgctgcctct |
| 961 | tggtgctttt gcctatggcc cctattctgc ctgctgaaga cactcttgcc agcatggact |
| 1021 | taaacccctc cagctctgac aatcctcttt ctcttttgtt ttacatgaag ggtctggcag |
| 1081 | ccaaagcaat cactcaaagt tcaaacctta tcattttttg ctttgttcct cttggccttg |
| 1141 | gttttgtaca tcagctttga aaataccatc ccagggttaa tgctggggtt aatttataac |
| 1201 | tgagagtgct ctagttctgc aatacaggac atgctataaa aatggaaaga tgttgctttc |
| 1261 | tgagagatca ggtggattct tgggcatttg cttaagcttg gtaccgccac catgggggag |
| 1321 | tttgggctga gctgggtctt tctggtggcc ctgctgaagg gagtccagtg tgagcagcag |
| 1381 | ggagcttcta gacctggacc tagagatgct caggctcatc ctggaagacc tagagctgtg |
| 1441 | cctacacagt gtgatgtgcc tcctaattct agatttgatt gtgctcctga taaggctata |
| 1501 | acacaggagc agtgtgaggc tagaggatgt tgttatattc ctgctaagca gggactgcag |
| 1561 | ggagctcaga tgggacagcc ttggtgtttt tttcctcctt cttatccttc ttataagctg |
| 1621 | gagaatctgt cttcttctga gatgggatat acagctacac tgacaagaac aacacctaca |
| 1681 | ttttttccta aggatattct gacactgaga ctggatgtga tgatggagac agagaataga |
| 1741 | ctgcatttta caattaagga tcctgctaat agaagatatg aggtgcctct agagacacct |
| 1801 | agagtgcatt ctagagctcc ttctcctctg tattctgtgg agttttctga ggagcctttt |
| 1861 | ggagtgattg tgcatagaca gctggatgga agagtgctgc tgaatacaac agtggctcct |
| 1921 | ctgttttttg ctgatcagtt tctgcagctg tctacatctc tgccttctca gtatattaca |
| 1981 | ggactggctg agcatctgtc tcctctgatg ctgtctacat cttggacaag aattacactg |
| 2041 | tggaatagag atctggctcc tacacctgga gctaatctgt atggatctca tcctttttat |
| 2101 | ctggctctgg aggatggagg atctgctcat ggagtgtttc tgctgaattc taatgctatg |
| 2161 | gatgtagtgc tgcagccttc tcctgctctg tcttggagat ctacaggagg aattctggat |
| 2221 | gtgtatattt ttctaggacc tgagcctaag tctgtggtgc agcagtatct ggatgtagtg |
| 2281 | ggatatcctt ttatgcctcc ttattgggga ctgggatttc atctgtgtag atggggatat |
| 2341 | tcttctacag ctattacaag acaggtggtt gagaatatga caagagctca ttttcctctg |
| 2401 | gatgtgcagt ggaatgatct agattatatg gattctagaa gagattttac atttaataag |
| 2461 | gatggattta gagattttcc tgctatggtg caggagctgc atcagggagg aagaagatat |
| 2521 | atgatgattg tggatcctgc tatttcttct tctggacctg ctggatctta tagaccttat |
| 2581 | gatgagggac tgagaagagg agtgtttatt acaaatgaga caggacagcc tctgattgga |
| 2641 | aaggtgtggc ctggatctac agcttttcct gattttacaa atcctacagc tctggcttgg |
| 2701 | tgggaggata tggtggctga gtttcatgat caggtgcctt ttgatggaat gtggattgat |
| 2761 | atgaatgagc cttctaattt tataagagga tctgaggatg gatgtcctaa taatgagcta |
| 2821 | gagaatcctc cttatgtgcc tggagtagtg ggaggaacac tgcaggctgc tacaatttgt |
| 2881 | gcttcttctc atcagtttct gtctacacat tataatctgc ataatctgta tggactgaca |
| 2941 | gaggctattg cttctcatag agctctggtg aaggctagag gaacaagacc ttttgtgatt |
| 3001 | tctagatcta catttgctgg acatggaaga tatgctggac attggacagg agatgtatgg |
| 3061 | tcttcttggg agcagctagc ttcttctgtg cctgagattc tgcagtttaa tctgctagga |
| 3121 | gtgcctctgg tgggagctga tgtgtgtgga tttctgggaa atacatctga ggagctgtgt |
| 3181 | gtgagatgga cacagctggg agctttttat ccttttatga gaaatcataa ttctctgctg |
| 3241 | tctctgcctc aggagcctta ttctttttct gagcctgctc agcaggctat gagaaaggct |
| 3301 | ctgacactga gatatgctct gctgcctcat ctgtatacac tgtttcatca ggctcatgtg |
| 3361 | gctggagaga cagtagctag acctctgttt ctggagtttc ctaaggattc ttctacatgg |
| 3421 | acagttgatc atcagctgct gtggggagag gctctgctga ttacacctgt gctgcaggct |
| 3481 | ggaaaggctg aggtgacagg atattttcct ctgggaacat ggtatgatct gcagacagtg |
| 3541 | cctatagagg ctctgggatc tctgcctcct cctcctgctg ctcctagaga gcctgctatt |
| 3601 | cattctgagg gacagtgggt gacactgcct gctcctctgg atacaattaa tgtgcatctg |
| 3661 | agagctggat atattattcc tctgcaggga cctggactga caacaacaga gtctagacag |
| 3721 | cagcctatgg ctctggctgt ggctctgaca aagggaggag aggctagagg agagctgttt |
| 3781 | tgggatgatg gagagtctct ggaggtgctg gagagaggag cttatacaca ggtgattttt |
| 3841 | ctggctagaa ataatacaat tgtgaatgag ctggtgagag tgacatctga gggagctgga |
| 3901 | ctgcagctgc agaaggtgac agtgctggga gtggctacag ctcctcagca ggtgctgtct |
| 3961 | aatggagtgc ctgtgtctaa ttttacatat tctcctgata caaaggtgct ggatatttgt |
| 4021 | gtgtctctgc tgatgggaga gcagtttctg gtgtcttggt gttaatgatt taaatctcga |
| 4081 | gccgggcgga gtgtgttagt ctctccagag ggaggctggt tccccaggga agcagagcct |
| 4141 | gtgtgcgggc agcagctgtg tgcgggcctg ggggttgtta agtgcaatta tttttaataa |
| 4201 | aaggggcatt tggaaaaaaa aaaaaaaggt agcagtcgac agatgaattc tgcagatctg |
| 4261 | tggcttctag ctgcccgggt ggcatccctg tgacccctcc ccagtgcctc tcctggccct |
| 4321 | ggaagttgcc actccagtgc ccaccagcct tgtcctaata aaattaagtt gcatcatttt |
| 4381 | gtctgactag gtgtccttct ataatattat ggggtggagg ggggtggtat ggagcaaggg |
| 4441 | gcaagttggg aagacaacct gtagggcctg cggggtctat tgggaaccaa gctggagtgc |
| 4501 | agtggcacaa tcttggctca ctgcaatctc cgcctcctgg gttcaagcga ttctcctgcc |
| 4561 | tcagcctccc gagttgttgg gattccaggc atgcatgacc aggctcagct aatttttgtt |
| 4621 | tttttggtag agacggggtt tcaccatatt ggccaggctg gtctccaact cctaatctca |
| 4681 | ggtgatctac ccaccttggc ctcccaaatt gctgggatta caggcgtgaa ccactgctcc |
| 4741 | cttccctgtc cttctgattt taaaaaaaag agaaatgttc tggcacctgc acttgcactg |
| 4801 | gggacagcct attttgctag tttgttttgt ttcgttttgt tttgatggag agcgtatgtt |
| 4861 | gtttaaacgc ggccgcgtag ataagtagca tggcgggtta atcattaact acaaggaacc |
| 4921 | cctagtgatg gagttggcca ctccctctct gcgcgctcgc tcgctcactg aggccgggcg |
| 4981 | accaaaggtc gcccgacgcc cgggctttgc ccgggcggcc tcagtgagcg agcgagcgcg |
| 5041 | cagttaatta aggcgcccta ggccgaccct tagactctgt actcagttct ataaacgagc |
| 5101 | cattggatac gagatccgta gattgataag ggacacggaa tatccccgga cgcaatagac |
| 5161 | accggtggac agcttggtat cctgagcaca gtcgcgcgtc cgaatctagc tctactttag |
| 5221 | aggccccgga ttctgatggt cgtagaccgc agaaccgatt ggggggatga gatctactag |
| 5281 | ttatcagcac acaattgccc attatacgcg cgtataatgg actattgtgt gctgatatag |
| 5341 | ggataacagg gtaattctag agctagcata tggatccatc gatttgatgc ggtattttct |
| 5401 | ccttacgcat ctgtgcggta tttcacaccg catacgtcaa agcaaccata gtacgcgccc |
| 5461 | tgtagcggcg cattaagcgc ggcgggtgtg gtggttacgc gcagcgtgac cgctacactt |
| 5521 | gccagcgccc tagcgcccgc tcctttcgct ttcttccctt cctttctcgc cacgttcgcc |
| 5581 | ggctttcccc gtcaagctct aaatcggggg ctccctttag ggttccgatt tagtgcttta |
| 5641 | cggcacctcg accccaaaaa acttgatttg ggtgatggtt cacgtagtgg gccatcgccc |
| 5701 | tgatagacgg tttttcgccc tttgacgttg gagtccacgt tctttaatag tggactcttg |
| 5761 | ttccaaactg gaacaacact caactctatc tcgggctatt cttttgattt ataagggatt |
| 5821 | ttgccgattt cggtctattg gttaaaaaat gagctgattt aacaaaaatt taacgcgaat |
| 5881 | tttaacaaaa tattaacgtt tacaatttta tggtgcactc tcagtacaat ctgctctgat |
| 5941 | gccgcatagt taagccagcc ccgacacccg ccaacacccg ctgacgcgcc ctgacgggct |
| 6001 | tgtctgctcc cggcatccgc ttacagacaa gctgtgaccg tctccgggag ctgcatgtgt |
| 6061 | cagaggtttt caccgtcatc accgaaacgc gcgagacgaa agggcctcgt gatacgccta |
| 6121 | tttttatagg ttaatgtcat gataataatg gtttcttaga cgtcaggtgg cacttttcgg |
| 6181 | ggaaatgtgc gcggaacccc tatttgttta tttttctaaa tacattcaaa tatgtatccg |
| 6241 | ctcatgagac aataaccctg ataaatgctt caataatatt gaaaaaggaa gagtatgagc |
| 6301 | catattcaac gggaaacgtc gaggccgcga ttaaattcca acatggatgc tgatttatat |
| 6361 | gggtataaat gggctcgcga taatgtcggg caatcaggtg cgacaatcta tcgcttgtat |
| 6421 | gggaagcccg atgcgccaga gttgtttctg aaacatggca aaggtagcgt tgccaatgat |
| 6481 | gttacagatg agatggtcag actaaactgg ctgacggaat ttatgcctct tccgaccatc |
| 6541 | aagcatttta tccgtactcc tgatgatgca tggttactca ccactgcgat ccccggaaaa |
| 6601 | acagcattcc aggtattaga agaatatcct gattcaggtg aaaatattgt tgatgcgctg |
| 6661 | gcagtgttcc tgcgccggtt gcattcgatt cctgtttgta attgtccttt taacagcgat |
| 6721 | cgcgtatttc gtctcgctca ggcgcaatca cgaatgaata acggtttggt tgatgcgagt |
| 6781 | gattttgatg acgagcgtaa tggctggcct gttgaacaag tctggaaaga aatgcataaa |
| 6841 | cttttgccat tctcaccgga ttcagtcgtc actcatggtg atttctcact tgataacctt |
| 6901 | atttttgacg aggggaaatt aataggttgt attgatgttg gacgagtcgg aatcgcagac |
| 6961 | cgataccagg atcttgccat cctatggaac tgcctcggtg agttttctcc ttcattacag |
| 7021 | aaacggcttt ttcaaaaata tggtattgat aatcctgata tgaataaatt gcagtttcat |
| 7081 | ttgatgctcg atgagttttt ctaagcgtat aatggtctag agctagcata tggatccatc |
| 7141 | gattccatta tacgcctgtc agaccaagtt tactcatata tactttagat tgatttaaaa |
| 7201 | cttcattttt aatttaaaag gatctaggtg aagatccttt ttgataatct catgaccaaa |
| 7261 | atcccttaac gtgagttttc gttccactga gcgtcagacc ccgtagaaaa gatcaaagga |
| 7321 | tcttcttgag atcctttttt tctgcgcgta atctgctgct tgcaaacaaa aaaaccaccg |
| 7381 | ctaccagcgg tggtttgttt gccggatcaa gagctaccaa ctctttttcc gaaggtaact |
| 7441 | ggcttcagca gagcgcagat accaaatact gttcttctag tgtagccgta gttaggccac |
| 7501 | cacttcaaga actctgtagc accgcctaca tacctcgctc tgctaatcct gttaccagtg |
| 7561 | gctgctgcca gtggcgataa gtcgtgtctt accgggttgg actcaagacg atagttaccg |
| 7621 | gataaggcgc agcggtcggg ctgaacgggg ggttcgtgca cacagcccag cttggagcga |
| 7681 | acgacctaca ccgaactgag atacctacag cgtgagctat gagaaagcgc cacgcttccc |
| 7741 | gaagggagaa aggcggacag gtatccggta agcggcaggg tcggaacagg agagcgcacg |
| 7801 | agggagcttc cagggggaaa cgcctggtat ctttatagtc ctgtcgggtt tcgccacctc |
| 7861 | tgacttgagc gtcgattttt gtgatgctcg tcaggggggc ggagcctatg gaaaaacgcc |
| 7921 | agcaacgcgg cctttttacg gttcctggcc ttttgctggc cttttgctca catgt |

| pP083_seq100-IL2wt_5-56 deletion (SEQ ID NO: 488) | |
|---|---|
| (SEQ ID NO: 488) | |
| 1 | tctagagcta gcatatggat ccatcgattt agggataaca gggtaattat cagcacacaa |
| 61 | ttgcccatta tacgcgcgta taatggacta ttgtgtgctg atatctgtac acttaagggc |
| 121 | tagatcttag cttacgtcac tagagggtcc acgtttagtt tttaagatcc attgatctcc |
| 181 | taaacgctgc aagattcgca acctggtata cttagcctag gcgctaggtc ctagtgcagc |
| 241 | gggacttttt ttctaaagtc gttgagagga ggagtcgtca gaccagatag ctttgatgtc |
| 301 | ctgatcggaa ggatcgttgg cccccctgca ggcagctgtt aattaactgc gcgctcgctc |
| 361 | gctcactgag gccgcccggg caaagcccgg gcgtcgggcg acctttggtc gcccggcctc |
| 421 | agtgagcgag cgagcgcgca gagagggagt ggccaactcc atcactaggg gttccttgta |
| 481 | gttaatgatt aacccgccat gctacttatc tacggcgcgc cacgcgttct aggagttaat |
| 541 | ttttaaaaag cagtcaaaag tccaagtgcc cttgcgagca tttactctct ctgtttgctc |
| 601 | tggttaataa tctcaggagc acaaacattc cttactagtt ctaggagtta atttttaaaa |
| 661 | agcagtcaaa agtccaagtg cccttgcgag catttactct ctctgtttgc tctggttaat |
| 721 | aatctcagga gcacaaacat tccttactag ttctagagcg gccgccagtg tgctggaatt |
| 781 | cggctttttt agggctggaa gctacctttg acatcatttc ctctgcgaat gcatgtataa |
| 841 | tttctacaga acctattaga aaggatcacc cagcctctgc ttttgtacaa ctttccctta |
| 901 | aaaaactgcc aatcccactg ctgtttggcc caatagtgag aactttttcc tgctgcctct |
| 961 | tggtgctttt gcctatggcc cctattctgc ctgctgaaga cactcttgcc agcatggact |
| 1021 | taaacccctc cagctctgac aatcctcttt ctcttttgtt ttacatgaag ggtctggcag |
| 1081 | ccaaagcaat cactcaaagt tcaaacctta tcattttttg ctttgttcct cttggccttg |
| 1141 | gttttgtaca tcagctttga aaataccatc ccagggttaa tgctggggtt aatttataac |
| 1201 | tgagagtgct ctagttctgc aatacaggac atgctataaa aatggaaaga tgttgctttc |
| 1261 | tgagagatca ggtggattct tgggcatttg cttaagcttg gtaccgccac catgggggtg |
| 1321 | aggtacagaa tgcagctgct gtcctgcatt gcactgtcac tggcactggt taccaactca |
| 1381 | cagcaggggg ccagcaggcc aggccccagg gatgcccagg cccaccctgg cagacccagg |
| 1441 | gctgtgccca cccagtgtga tgtgcctccc aacagcagat ttgactgtgc ccctgataag |
| 1501 | gccattactc aggaacagtg tgaggccagg ggctgctgct atatccctgc caagcagggc |
| 1561 | ctgcaggggg cccagatggg gcagccctgg tgcttcttcc cccccagcta cccctcttat |
| 1621 | aagctggaga atctgagcag ctctgaaatg ggctacactg ccactctgac caggactacc |
| 1681 | cccaccttct tccccaagga tattctgact ctgaggctgg atgtgatgat ggagactgag |
| 1741 | aacaggctgc acttcaccat caaggaccct gccaacagga ggtatgaggt gcccctggaa |
| 1801 | actcccaggg tgcactctag ggcccccagc cccctgtatt ctgtggagtt ctctgaggag |
| 1861 | ccttttgggg tcattgtcca caggcagctg gatggcaggg tgctgctgaa tactactgtg |
| 1921 | gcccctctgt tctttgctga ccagttcctg cagctgagca ccagcctgcc ttctcagtac |
| 1981 | attactggcc tggctgagca tctgagcccc ctgatgctga gcacctcttg gaccagaatc |
| 2041 | accctgtgga acagggacct ggctcccact cctggggcca acctgtatgg cagccacccc |
| 2101 | ttctacctgg ccctggagga tgggggctct gcccatgggg tgttcctgct gaacagcaat |
| 2161 | gccatggatg tggtgctgca gccctctcct gccctgtctt ggagatctac tgggggcatc |
| 2221 | ctggatgtgt atatcttcct ggggcctgag cccaagtctg tggtgcagca gtacctggat |
| 2281 | gtggtgggct accccttcat gcccccctac tggggcctgg gcttccacct gtgcaggtgg |
| 2341 | ggctactctt ctactgctat caccaggcag gtggtggaga acatgaccag ggctcacttc |
| 2401 | cctctggatg tgcagtggaa tgacctggac tacatggact ctaggagaga cttcactttt |
| 2461 | aataaggatg ggttcaggga ctttcctgcc atggtgcagg agctgcatca ggggggcagg |
| 2521 | agatatatga tgattgtgga ccctgctatt tctagctctg gccctgctgg cagctatagg |
| 2581 | ccctatgatg aggggctgag gaggggggtg ttcatcacta atgagactgg ccagcccctg |
| 2641 | attggcaagg tgtggcctgg ctctactgcc ttccctgatt tcaccaaccc cactgccctg |
| 2701 | gcctggtggg aggatatggt ggctgagttt catgaccagg tgccctttga tggcatgtgg |
| 2761 | attgacatga atgagcccag caactttatc aggggctctg aagatggctg ccccaacaat |
| 2821 | gagctggaga acccccccta tgtgcctggg gtggtggggg gcaccctgca ggctgccacc |
| 2881 | atctgtgcca gcagccacca gttcctgagc acccactata acctgcacaa cctgtatggc |
| 2941 | ctgactgagg ccattgcctc tcacagggcc ctggtgaagg ctagggggac taggcccttt |
| 3001 | gtgatcagca ggtctacttt tgctggccat ggcaggtatg ctgggcactg gactggggat |
| 3061 | gtgtggtcta gctgggagca gctggccagc tctgtgcctg agatcctgca gtttaatctg |
| 3121 | ctgggggtgc ccctggtggg ggctgatgtg tgtggcttcc tgggcaatac ctctgaggag |
| 3181 | ctgtgtgtga ggtggactca gctgggggct ttctacccct tcatgagaaa ccacaactct |
| 3241 | ctgctgagcc tgccccagga gccctattct ttttctgagc ctgcccagca ggctatgagg |
| 3301 | aaggccctga ctctgaggta tgccctgctg ccccacctgt ataccctgtt ccatcaggcc |
| 3361 | catgtggctg gggagactgt ggccagacct ctgttcctgg agttccccaa ggatagctct |
| 3421 | acttggactg tggaccacca gctgctgtgg ggggaggctc tgctgatcac ccctgtgctg |
| 3481 | caggctggga aggctgaggt gactggctat ttccccctgg gcacctggta tgatctgcag |
| 3541 | actgtgccca ttgaggccct gggctctctg ccccctcccc ctgctgcccc cagggagcct |
| 3601 | gccatccact ctgagggcca gtgggtgacc ctgcctgccc ctctggacac tatcaatgtg |
| 3661 | cacctgaggg ctggctacat catccccctg cagggccctg gcctgactac cactgagtct |
| 3721 | aggcagcagc ccatggccct ggctgtggct ctgaccaagg ggggggaggc caggggggag |
| 3781 | ctgttctggg atgatgggga gtctctggag gtgctggaga ggggggccta cacccaggtg |
| 3841 | atcttcctgg ctaggaataa caccattgtc aatgagctgg tgagggtgac ctctgagggg |
| 3901 | gctggcctgc agctgcagaa ggtgactgtg ctgggggtgg ctactgcccc ccagcaggtg |
| 3961 | ctgagcaatg gggtgcctgt gagcaacttc acctatagcc ctgacaccaa ggtgctggac |
| 4021 | atctgtgtga gcctgctgat gggggagcag ttcctggtga gctggtgcta atttaaatct |
| 4081 | cgagccgggc ggagtgtgtt agtctctcca gagggaggct ggttccccag ggaagcagag |
| 4141 | cctgtgtgcg ggcagcagct gtgtgcgggc ctgggggttg ttaagtgcaa ttatttttaa |
| 4201 | taaaaggggc atttggaaaa aaaaaaaaaa ggtagcagtc gacagatgaa ttctgcagat |
| 4261 | ctgtggcttc tagctgcccg ggtggcatcc ctgtgacccc tccccagtgc ctctcctggc |
| 4321 | cctggaagtt gccactccag tgcccaccag ccttgtccta ataaaattaa gttgcatcat |
| 4381 | tttgtctgac taggtgtcct tctataatat tatggggtgg aggggggtgg tatggagcaa |
| 4441 | ggggcaagtt gggaagacaa cctgtagggc ctgcggggtc tattgggaac caagctggag |
| 4501 | tgcagtggca caatcttggc tcactgcaat ctccgcctcc tgggttcaag cgattctcct |
| 4561 | gcctcagcct cccgagttgt tgggattcca ggcatgcatg accaggctca gctaattttt |
| 4621 | gtttttttgg tagagacggg gtttcaccat attggccagg ctggtctcca actcctaatc |
| 4681 | tcaggtgatc tacccacctt ggcctcccaa attgctggga ttacaggcgt gaaccactgc |
| 4741 | tcccttccct gtccttctga ttttaaaaaa aagagaaatg ttctggcacc tgcacttgca |
| 4801 | ctggggacag cctattttgc tagtttgttt tgtttcgttt tgttttgatg gagagcgtat |
| 4861 | gttgtttaaa cgcggccgcg tagataagta gcatggcggg ttaatcatta actacaagga |
| 4921 | acccctagtg atggagttgg ccactccctc tctgcgcgct cgctcgctca ctgaggccgg |
| 4981 | gcgaccaaag gtcgcccgac gcccgggctt tgcccgggcg gcctcagtga gcgagcgagc |
| 5041 | gcgcagttaa ttaaggcgcc ctaggccgac ccttagactc tgtactcagt tctataaacg |
| 5101 | agccattgga tacgagatcc gtagattgat aagggacacg gaatatcccc ggacgcaata |
| 5161 | gacaccggtg gacagcttgg tatcctgagc acagtcgcgc gtccgaatct agctctactt |
| 5221 | tagaggcccc ggattctgat ggtcgtagac cgcagaaccg attgggggga tgagatctac |
| 5281 | tagttatcag cacacaattg cccattatac gcgcgtataa tggactattg tgtgctgata |
| 5341 | tagggataac agggtaattc tagagctagc atatggatcc atcgatttga tgcggtattt |
| 5401 | tctccttacg catctgtgcg gtatttcaca ccgcatacgt caaagcaacc atagtacgcg |
| 5461 | ccctgtagcg gcgcattaag cgcggcgggt gtggtggtta cgcgcagcgt gaccgctaca |
| 5521 | cttgccagcg ccctagcgcc cgctcctttc gctttcttcc cttcctttct cgccacgttc |
| 5581 | gccggctttc cccgtcaagc tctaaatcgg gggctccctt tagggttccg atttagtgct |
| 5641 | ttacggcacc tcgaccccaa aaaacttgat ttgggtgatg gttcacgtag tgggccatcg |
| 5701 | ccctgataga cggtttttcg ccctttgacg ttggagtcca cgttctttaa tagtggactc |
| 5761 | ttgttccaaa ctggaacaac actcaactct atctcgggct attcttttga tttataaggg |
| 5821 | attttgccga tttcggtcta ttggttaaaa aatgagctga tttaacaaaa atttaacgcg |
| 5881 | aattttaaca aaatattaac gtttacaatt ttatggtgca ctctcagtac aatctgctct |
| 5941 | gatgccgcat agttaagcca gccccgacac ccgccaacac ccgctgacgc gccctgacgg |
| 6001 | gcttgtctgc tcccggcatc cgcttacaga caagctgtga ccgtctccgg gagctgcatg |
| 6061 | tgtcagaggt tttcaccgtc atcaccgaaa cgcgcgagac gaaagggcct cgtgatacgc |
| 6121 | ctatttttat aggttaatgt catgataata atggtttctt agacgtcagg tggcactttt |
| 6181 | cggggaaatg tgcgcggaac ccctatttgt ttatttttct aaatacattc aaatatgtat |
| 6241 | ccgctcatga gacaataacc ctgataaatg cttcaataat attgaaaaag gaagagtatg |
| 6301 | agccatattc aacgggaaac gtcgaggccg cgattaaatt ccaacatgga tgctgattta |
| 6361 | tatgggtata aatgggctcg cgataatgtc gggcaatcag gtgcgacaat ctatcgcttg |
| 6421 | tatgggaagc ccgatgcgcc agagttgttt ctgaaacatg gcaaaggtag cgttgccaat |
| 6481 | gatgttacag atgagatggt cagactaaac tggctgacgg aatttatgcc tcttccgacc |
| 6541 | atcaagcatt ttatccgtac tcctgatgat gcatggttac tcaccactgc gatccccgga |
| 6601 | aaaacagcat tccaggtatt agaagaatat cctgattcag gtgaaaatat tgttgatgcg |
| 6661 | ctggcagtgt tcctgcgccg gttgcattcg attcctgttt gtaattgtcc ttttaacagc |
| 6721 | gatcgcgtat ttcgtctcgc tcaggcgcaa tcacgaatga ataacggttt ggttgatgcg |
| 6781 | agtgattttg atgacgagcg taatggctgg cctgttgaac aagtctggaa agaaatgcat |
| 6841 | aaacttttgc cattctcacc ggattcagtc gtcactcatg gtgatttctc acttgataac |
| 6901 | cttatttttg acgaggggaa attaataggt tgtattgatg ttggacgagt cggaatcgca |
| 6961 | gaccgatacc aggatcttgc catcctatgg aactgcctcg gtgagttttc tccttcatta |
| 7021 | cagaaacggc tttttcaaaa atatggtatt gataatcctg atatgaataa attgcagttt |
| 7081 | catttgatgc tcgatgagtt tttctaagcg tataatggtc tagagctagc atatggatcc |
| 7141 | atcgattcca ttatacgcct gtcagaccaa gtttactcat atatacttta gattgattta |
| 7201 | aaacttcatt tttaatttaa aaggatctag gtgaagatcc tttttgataa tctcatgacc |
| 7261 | aaaatccctt aacgtgagtt ttcgttccac tgagcgtcag accccgtaga aaagatcaaa |
| 7321 | ggatcttctt gagatccttt ttttctgcgc gtaatctgct gcttgcaaac aaaaaaacca |
| 7381 | ccgctaccag cggtggtttg tttgccggat caagagctac caactctttt tccgaaggta |
| 7441 | actggcttca gcagagcgca gataccaaat actgttcttc tagtgtagcc gtagttaggc |
| 7501 | caccacttca agaactctgt agcaccgcct acatacctcg ctctgctaat cctgttacca |
| 7561 | gtggctgctg ccagtggcga taagtcgtgt cttaccgggt tggactcaag acgatagtta |
| 7621 | ccggataagg cgcagcggtc gggctgaacg gggggttcgt gcacacagcc cagcttggag |
| 7681 | cgaacgacct acaccgaact gagataccta cagcgtgagc tatgagaaag cgccacgctt |
| 7741 | cccgaaggga gaaagcggga caggtatccg gtaagcggca gggtcggaac aggagagcgc |
| 7801 | acgagggagc ttccaggggg aaacgcctgg tatctttata gtcctgtcgg gtttcgccac |
| 7861 | ctctgacttg agcgtcgatt tttgtgatgc tcgtcagggg ggcggagcct atggaaaaac |
| 7921 | gccagcaacg cggccttttt acggttcctg gccttttgct ggccttttgc tcacatgt |

| pP084_seq100-201Ig_5-56 deletion (SEQ ID NO: 489) | |
|---|---|
| (SEQ ID NO: 489) | |
| 1 | tctagagcta gcatatggat ccatcgattt agggataaca gggtaattat cagcacacaa |
| 61 | ttgcccatta tacgcgcgta taatggacta ttgtgtgctg atatctgtac acttaagggc |
| 121 | tagatcttag cttacgtcac tagagggtcc acgtttagtt tttaagatcc attgatctcc |
| 181 | taaacgctgc aagattcgca acctggtata cttagcctag gcgctaggtc ctagtgcagc |
| 241 | gggacttttt ttctaaagtc gttgagagga ggagtcgtca gaccagatag ctttgatgtc |
| 301 | ctgatcggaa ggatcgttgg cccccctgca ggcagctgtt aattaactgc gcgctcgctc |
| 361 | gctcactgag gccgcccggg caaagcccgg gcgtcgggcg acctttggtc gcccggcctc |
| 421 | agtgagcgag cgagcgcgca gagagggagt ggccaactcc atcactaggg gttccttgta |
| 481 | gttaatgatt aacccgccat gctacttatc tacggcgcgc cacgcgttct aggagttaat |
| 541 | ttttaaaaag cagtcaaaag tccaagtgcc cttgcgagca tttactctct ctgtttgctc |
| 601 | tggttaataa tctcaggagc acaaacattc cttactagtt ctaggagtta atttttaaaa |
| 661 | agcagtcaaa agtccaagtg cccttgcgag catttactct ctctgtttgc tctggttaat |
| 721 | aatctcagga gcacaaacat tccttactag ttctagagcg gccgccagtg tgctggaatt |
| 781 | cggctttttt agggctggaa gctacctttg acatcatttc ctctgcgaat gcatgtataa |
| 841 | tttctacaga acctattaga aaggatcacc cagcctctgc ttttgtacaa ctttccctta |
| 901 | aaaaactgcc aatcccactg ctgtttggcc caatagtgag aactttttcc tgctgcctct |
| 961 | tggtgctttt gcctatggcc cctattctgc ctgctgaaga cactcttgcc agcatggact |
| 1021 | taaacccctc cagctctgac aatcctcttt ctcttttgtt ttacatgaag ggtctggcag |
| 1081 | ccaaagcaat cactcaaagt tcaaacctta tcattttttg ctttgttcct cttggccttg |
| 1141 | gttttgtaca tcagctttga aaataccatc ccagggttaa tgctggggtt aatttataac |
| 1201 | tgagagtgct ctagttctgc aatacaggac atgctataaa aatggaaaga tgttgctttc |
| 1261 | tgagagatca ggtggattct tgggcatttg cttaagcttg gtaccgccac catgggggtg |
| 1321 | agggagtttg ggctgagctg ggtctttctg gtggccctgc tgaagggagt ccagtgtgag |
| 1381 | cagcaggggg ccagcaggcc aggccccagg gatgcccagg cccaccctgg cagacccagg |
| 1441 | gctgtgccca cccagtgtga tgtgcctccc aacagcagat ttgactgtgc ccctgataag |
| 1501 | gccattactc aggaacagtg tgaggccagg ggctgctgct atatccctgc caagcagggc |
| 1561 | ctgcaggggg cccagatggg gcagccctgg tgcttcttcc cccccagcta cccctcttat |
| 1621 | aagctggaga atctgagcag ctctgaaatg ggctacactg ccactctgac caggactacc |
| 1681 | cccaccttct tccccaagga tattctgact ctgaggctgg atgtgatgat ggagactgag |
| 1741 | aacaggctgc acttcaccat caaggaccct gccaacagga ggtatgaggt gcccctggaa |
| 1801 | actcccaggg tgcactctag ggcccccagc cccctgtatt ctgtggagtt ctctgaggag |
| 1861 | ccttttgggg tcattgtcca caggcagctg gatggcaggg tgctgctgaa tactactgtg |
| 1921 | gcccctctgt tctttgctga ccagttcctg cagctgagca ccagcctgcc ttctcagtac |
| 1981 | attactggcc tggctgagca tctgagcccc ctgatgctga gcacctcttg gaccagaatc |
| 2041 | accctgtgga acagggacct ggctcccact cctggggcca acctgtatgg cagccacccc |
| 2101 | ttctacctgg ccctggagga tgggggctct gcccatgggg tgttcctgct gaacagcaat |
| 2161 | gccatggatg tggtgctgca gccctctcct gccctgtctt ggagatctac tgggggcatc |
| 2221 | ctggatgtgt atatcttcct ggggcctgag cccaagtctg tggtgcagca gtacctggat |
| 2281 | gtggtgggct accccttcat gcccccctac tggggcctgg gcttccacct gtgcaggtgg |
| 2341 | ggctactctt ctactgctat caccaggcag gtggtggaga acatgaccag ggctcacttc |
| 2401 | cctctggatg tgcagtggaa tgacctggac tacatggact ctaggagaga cttcactttt |
| 2461 | aataaggatg ggttcaggga ctttcctgcc atggtgcagg agctgcatca ggggggcagg |
| 2521 | agatatatga tgattgtgga ccctgctatt tctagctctg gccctgctgg cagctatagg |
| 2581 | ccctatgatg aggggctgag gaggggggtg ttcatcacta atgagactgg ccagcccctg |
| 2641 | attggcaagg tgtggcctgg ctctactgcc ttccctgatt tcaccaaccc cactgccctg |
| 2701 | gcctggtggg aggatatggt ggctgagttt catgaccagg tgccctttga tggcatgtgg |
| 2761 | attgacatga atgagcccag caactttatc aggggctctg aagatggctg ccccaacaat |
| 2821 | gagctggaga acccccccta tgtgcctggg gtggtggggg gcaccctgca ggctgccacc |
| 2881 | atctgtgcca gcagccacca gttcctgagc acccactata acctgcacaa cctgtatggc |
| 2941 | ctgactgagg ccattgcctc tcacagggcc ctggtgaagg ctagggggac taggcccttt |
| 3001 | gtgatcagca ggtctacttt tgctggccat ggcaggtatg ctgggcactg gactggggat |
| 3061 | gtgtggtcta gctgggagca gctggccagc tctgtgcctg agatcctgca gtttaatctg |
| 3121 | ctgggggtgc ccctggtggg ggctgatgtg tgtggcttcc tgggcaatac ctctgaggag |
| 3181 | ctgtgtgtga ggtggactca gctgggggct ttctacccct tcatgagaaa ccacaactct |
| 3241 | ctgctgagcc tgccccagga gccctattct ttttctgagc ctgcccagca ggctatgagg |
| 3301 | aaggccctga ctctgaggta tgccctgctg ccccacctgt ataccctgtt ccatcaggcc |
| 3361 | catgtggctg gggagactgt ggccagacct ctgttcctgg agttccccaa ggatagctct |
| 3421 | acttggactg tggaccacca gctgctgtgg ggggaggctc tgctgatcac ccctgtgctg |
| 3481 | caggctggga aggctgaggt gactggctat ttccccctgg gcacctggta tgatctgcag |
| 3541 | actgtgccca ttgaggccct gggctctctg ccccctcccc ctgctgcccc cagggagcct |
| 3601 | gccatccact ctgagggcca gtgggtgacc ctgcctgccc ctctggacac tatcaatgtg |
| 3661 | cacctgaggg ctggctacat catccccctg cagggccctg gcctgactac cactgagtct |
| 3721 | aggcagcagc ccatggccct ggctgtggct ctgaccaagg ggggggaggc caggggggag |
| 3781 | ctgttctggg atgatgggga gtctctggag gtgctggaga ggggggccta cacccaggtg |
| 3841 | atcttcctgg ctaggaataa caccattgtc aatgagctgg tgagggtgac ctctgagggg |
| 3901 | gctggcctgc agctgcagaa ggtgactgtg ctgggggtgg ctactgcccc ccagcaggtg |
| 3961 | ctgagcaatg gggtgcctgt gagcaacttc acctatagcc ctgacaccaa ggtgctggac |
| 4021 | atctgtgtga gcctgctgat gggggagcag ttcctggtga gctggtgcta atttaaatct |
| 4081 | cgagccgggc ggagtgtgtt agtctctcca gagggaggct ggttccccag ggaagcagag |
| 4141 | cctgtgtgcg ggcagcagct gtgtgcgggc ctgggggttg ttaagtgcaa ttatttttaa |
| 4201 | taaaaggggc atttggaaaa aaaaaaaaaa ggtagcagtc gacagatgaa ttctgcagat |
| 4261 | ctgtggcttc tagctgcccg ggtggcatcc ctgtgacccc tccccagtgc ctctcctggc |
| 4321 | cctggaagtt gccactccag tgcccaccag ccttgtccta ataaaattaa gttgcatcat |
| 4381 | tttgtctgac taggtgtcct tctataatat tatggggtgg aggggggtgg tatggagcaa |
| 4441 | ggggcaagtt gggaagacaa cctgtagggc ctgcggggtc tattgggaac caagctggag |
| 4501 | tgcagtggca caatcttggc tcactgcaat ctccgcctcc tgggttcaag cgattctcct |
| 4561 | gcctcagcct cccgagttgt tgggattcca ggcatgcatg accaggctca gctaattttt |
| 4621 | gtttttttgg tagagacggg gtttcaccat attggccagg ctggtctcca actcctaatc |
| 4681 | tcaggtgatc tacccacctt ggcctcccaa attgctggga ttacaggcgt gaaccactgc |
| 4741 | tcccttccct gtccttctga ttttaaaaaa aagagaaatg ttctggcacc tgcacttgca |
| 4801 | ctggggacag cctattttgc tagtttgttt tgtttcgttt tgttttgatg gagagcgtat |
| 4861 | gttgtttaaa cgcggccgcg tagataagta gcatggcggg ttaatcatta actacaagga |
| 4921 | acccctagtg atggagttgg ccactccctc tctgcgcgct cgctcgctca ctgaggccgg |
| 4981 | gcgaccaaag gtcgcccgac gcccgggctt tgcccgggcg gcctcagtga gcgagcgagc |
| 5041 | gcgcagttaa ttaaggcgcc ctaggccgac ccttagactc tgtactcagt tctataaacg |
| 5101 | agccattgga tacgagatcc gtagattgat aagggacacg gaatatcccc ggacgcaata |
| 5161 | gacaccggtg gacagcttgg tatcctgagc acagtcgcgc gtccgaatct agctctactt |
| 5221 | tagaggcccc ggattctgat ggtcgtagac cgcagaaccg attgggggga tgagatctac |
| 5281 | tagttatcag cacacaattg cccattatac gcgcgtataa tggactattg tgtgctgata |
| 5341 | tagggataac agggtaattc tagagctagc atatggatcc atcgatttga tgcggtattt |
| 5401 | tctccttacg catctgtgcg gtatttcaca ccgcatacgt caaagcaacc atagtacgcg |
| 5461 | ccctgtagcg gcgcattaag cgcggcgggt gtggtggtta cgcgcagcgt gaccgctaca |
| 5521 | cttgccagcg ccctagcgcc cgctcctttc gctttcttcc cttcctttct cgccacgttc |
| 5581 | gccggctttc cccgtcaagc tctaaatcgg gggctccctt tagggttccg atttagtgct |
| 5641 | ttacggcacc tcgaccccaa aaaacttgat ttgggtgatg gttcacgtag tgggccatcg |
| 5701 | ccctgataga cggtttttcg ccctttgacg ttggagtcca cgttctttaa tagtggactc |
| 5761 | ttgttccaaa ctggaacaac actcaactct atctcgggct attcttttga tttataaggg |
| 5821 | attttgccga tttcggtcta ttggttaaaa aatgagctga tttaacaaaa atttaacgcg |
| 5881 | aattttaaca aaatattaac gtttacaatt ttatggtgca ctctcagtac aatctgctct |
| 5941 | gatgccgcat agttaagcca gccccgacac ccgccaacac ccgctgacgc gccctgacgg |
| 6001 | gcttgtctgc tcccggcatc cgcttacaga caagctgtga ccgtctccgg gagctgcatg |
| 6061 | tgtcagaggt tttcaccgtc atcaccgaaa cgcgcgagac gaaagggcct cgtgatacgc |
| 6121 | ctatttttat aggttaatgt catgataata atggtttctt agacgtcagg tggcactttt |
| 6181 | cggggaaatg tgcgcggaac ccctatttgt ttatttttct aaatacattc aaatatgtat |
| 6241 | ccgctcatga gacaataacc ctgataaatg cttcaataat attgaaaaag gaagagtatg |
| 6301 | agccatattc aacgggaaac gtcgaggccg cgattaaatt ccaacatgga tgctgattta |
| 6361 | tatgggtata aatgggctcg cgataatgtc gggcaatcag gtgcgacaat ctatcgcttg |
| 6421 | tatgggaagc ccgatgcgcc agagttgttt ctgaaacatg gcaaaggtag cgttgccaat |
| 6481 | gatgttacag atgagatggt cagactaaac tggctgacgg aatttatgcc tcttccgacc |
| 6541 | atcaagcatt ttatccgtac tcctgatgat gcatggttac tcaccactgc gatccccgga |
| 6601 | aaaacagcat tccaggtatt agaagaatat cctgattcag gtgaaaatat tgttgatgcg |
| 6661 | ctggcagtgt tcctgcgccg gttgcattcg attcctgttt gtaattgtcc ttttaacagc |
| 6721 | gatcgcgtat ttcgtctcgc tcaggcgcaa tcacgaatga ataacggttt ggttgatgcg |
| 6781 | agtgattttg atgacgagcg taatggctgg cctgttgaac aagtctggaa agaaatgcat |
| 6841 | aaacttttgc cattctcacc ggattcagtc gtcactcatg gtgatttctc acttgataac |
| 6901 | cttatttttg acgaggggaa attaataggt tgtattgatg ttggacgagt cggaatcgca |
| 6961 | gaccgatacc aggatcttgc catcctatgg aactgcctcg gtgagttttc tccttcatta |
| 7021 | cagaaacggc tttttcaaaa atatggtatt gataatcctg atatgaataa attgcagttt |
| 7081 | catttgatgc tcgatgagtt tttctaagcg tataatggtc tagagctagc atatggatcc |
| 7141 | atcgattcca ttatacgcct gtcagaccaa gtttactcat atatacttta gattgattta |
| 7201 | aaacttcatt tttaatttaa aaggatctag gtgaagatcc tttttgataa tctcatgacc |
| 7261 | aaaatccctt aacgtgagtt ttcgttccac tgagcgtcag accccgtaga aaagatcaaa |
| 7321 | ggatcttctt gagatccttt ttttctgcgc gtaatctgct gcttgcaaac aaaaaaacca |
| 7381 | ccgctaccag cggtggtttg tttgccggat caagagctac caactctttt tccgaaggta |
| 7441 | actggcttca gcagagcgca gataccaaat actgttcttc tagtgtagcc gtagttaggc |
| 7501 | caccacttca agaactctgt agcaccgcct acatacctcg ctctgctaat cctgttacca |
| 7561 | gtggctgctg ccagtggcga taagtcgtgt cttaccgggt tggactcaag acgatagtta |
| 7621 | ccggataagg cgcagcggtc gggctgaacg gggggttcgt gcacacagcc cagcttggag |
| 7681 | cgaacgacct acaccgaact gagataccta cagcgtgagc tatgagaaag cgccacgctt |
| 7741 | cccgaaggga gaaaggcgga caggtatccg gtaagcggca gggtcggaac aggagagcgc |
| 7801 | acgagggagc ttccaggggg aaacgcctgg tatctttata gtcctgtcgg gtttcgccac |
| 7861 | ctctgacttg agcgtcgatt tttgtgatgc tcgtcagggg ggcggagcct atggaaaaac |
| 7921 | gccagcaacg cggccttttt acggttcctg gccttttgct ggccttttgc tcacatgt |

| pP085_seq3-IL2wt_5-56 deletion (SEQ ID NO: 490) | |
|---|---|
| (SEQ ID NO: 490) | |
| 1 | tctagagcta gcatatggat ccatcgattt agggataaca gggtaattat cagcacacaa |
| 61 | ttgcccatta tacgcgcgta taatggacta ttgtgtgctg atatctgtac acttaagggc |
| 121 | tagatcttag cttacgtcac tagagggtcc acgtttagtt tttaagatcc attgatctcc |
| 181 | taaacgctgc aagattcgca acctggtata cttagcctag gcgctaggtc ctagtgcagc |
| 241 | gggacttttt ttctaaagtc gttgagagga ggagtcgtca gaccagatag ctttgatgtc |
| 301 | ctgatcggaa ggatcgttgg cccccctgca ggcagctgtt aattaactgc gcgctcgctc |
| 361 | gctcactgag gccgcccggg caaagcccgg gcgtcgggcg acctttggtc gcccggcctc |
| 421 | agtgagcgag cgagcgcgca gagagggagt ggccaactcc atcactaggg gttccttgta |
| 481 | gttaatgatt aacccgccat gctacttatc tacggcgcgc cacgcgttct aggagttaat |
| 541 | ttttaaaaag cagtcaaaag tccaagtgcc cttgcgagca tttactctct ctgtttgctc |
| 601 | tggttaataa tctcaggagc acaaacattc cttactagtt ctaggagtta atttttaaaa |
| 661 | agcagtcaaa agtccaagtg cccttgcgag catttactct ctctgtttgc tctggttaat |
| 721 | aatctcagga gcacaaacat tccttactag ttctagagcg gccgccagtg tgctggaatt |
| 781 | cggctttttt agggctggaa gctacctttg acatcatttc ctctgcgaat gcatgtataa |
| 841 | tttctacaga acctattaga aaggatcacc cagcctctgc ttttgtacaa ctttccctta |
| 901 | aaaaactgcc aatcccactg ctgtttggcc caatagtgag aactttttcc tgctgcctct |
| 961 | tggtgctttt gcctatggcc cctattctgc ctgctgaaga cactcttgcc agcatggact |
| 1021 | taaacccctc cagctctgac aatcctcttt ctcttttgtt ttacatgaag ggtctggcag |
| 1081 | ccaaagcaat cactcaaagt tcaaacctta tcattttttg ctttgttcct cttggccttg |
| 1141 | gttttgtaca tcagctttga aaataccatc ccagggttaa tgctggggtt aatttataac |
| 1201 | tgagagtgct ctagttctgc aatacaggac atgctataaa aatggaaaga tgttgctttc |
| 1261 | tgagagatca ggtggattct tgggcatttg cttaagcttg gtaccgccac catgggggtg |
| 1321 | aggtacagaa tgcagctgct gtcctgcatt gcactgtcac tggcactggt taccaactca |
| 1381 | cagcagggag cttctagacc tggacctaga gatgctcagg ctcatcctgg aagacctaga |
| 1441 | gctgtgccta cacagtgtga tgtgcctcct aattctagat ttgattgtgc tcctgataag |
| 1501 | gctataacac aggagcagtg tgaggctaga ggatgttgtt atattcctgc taagcaggga |
| 1561 | ctgcagggag ctcagatggg acagccttgg tgtttttttc ctccttctta tccttcttat |
| 1621 | aagctggaga atctgtcttc ttctgagatg ggatatacag ctacactgac aagaacaaca |
| 1681 | cctacatttt ttcctaagga tattctgaca ctgagactgg atgtgatgat ggagacagag |
| 1741 | aatagactgc attttacaat taaggatcct gctaatagaa gatatgaggt gcctctagag |
| 1801 | acacctagag tgcattctag agctccttct cctctgtatt ctgtggagtt ttctgaggag |
| 1861 | ccttttggag tgattgtgca tagacagctg gatggaagag tgctgctgaa tacaacagtg |
| 1921 | gctcctctgt tttttgctga tcagtttctg cagctgtcta catctctgcc ttctcagtat |
| 1981 | attacaggac tggctgagca tctgtctcct ctgatgctgt ctacatcttg gacaagaatt |
| 2041 | acactgtgga atagagatct ggctcctaca cctggagcta atctgtatgg atctcatcct |
| 2101 | ttttatctgg ctctggagga tggaggatct gctcatggag tgtttctgct gaattctaat |
| 2161 | gctatggatg tagtgctgca gccttctcct gctctgtctt ggagatctac aggaggaatt |
| 2221 | ctggatgtgt atatttttct aggacctgag cctaagtctg tggtgcagca gtatctggat |
| 2281 | gtagtgggat atccttttat gcctccttat tggggactgg gatttcatct gtgtagatgg |
| 2341 | ggatattctt ctacagctat tacaagacag gtggttgaga atatgacaag agctcatttt |
| 2401 | cctctggatg tgcagtggaa tgatctagat tatatggatt ctagaagaga ttttacattt |
| 2461 | aataaggatg gatttagaga ttttcctgct atggtgcagg agctgcatca gggaggaaga |
| 2521 | agatatatga tgattgtgga tcctgctatt tcttcttctg gacctgctgg atcttataga |
| 2581 | ccttatgatg agggactgag aagaggagtg tttattacaa atgagacagg acagcctctg |
| 2641 | attggaaagg tgtggcctgg atctacagct tttcctgatt ttacaaatcc tacagctctg |
| 2701 | gcttggtggg aggatatggt ggctgagttt catgatcagg tgccttttga tggaatgtgg |
| 2761 | attgatatga atgagccttc taattttata agaggatctg aggatggatg tcctaataat |
| 2821 | gagctagaga atcctcctta tgtgcctgga gtagtgggag gaacactgca ggctgctaca |
| 2881 | atttgtgctt cttctcatca gtttctgtct acacattata atctgcataa tctgtatgga |
| 2941 | ctgacagagg ctattgcttc tcatagagct ctggtgaagg ctagaggaac aagacctttt |
| 3001 | gtgatttcta gatctacatt tgctggacat ggaagatatg ctggacattg gacaggagat |
| 3061 | gtatggtctt cttgggagca gctagcttct tctgtgcctg agattctgca gtttaatctg |
| 3121 | ctaggagtgc ctctggtggg agctgatgtg tgtggatttc tgggaaatac atctgaggag |
| 3181 | ctgtgtgtga gatggacaca gctgggagct ttttatcctt ttatgagaaa tcataattct |
| 3241 | ctgctgtctc tgcctcagga gccttattct ttttctgagc ctgctcagca ggctatgaga |
| 3301 | aaggctctga cactgagata tgctctgctg cctcatctgt atacactgtt tcatcaggct |
| 3361 | catgtggctg gagagacagt agctagacct ctgtttctgg agtttcctaa ggattcttct |
| 3421 | acatggacag ttgatcatca gctgctgtgg ggagaggctc tgctgattac acctgtgctg |
| 3481 | caggctggaa aggctgaggt gacaggatat tttcctctgg gaacatggta tgatctgcag |
| 3541 | acagtgccta tagaggctct gggatctctg cctcctcctc ctgctgctcc tagagagcct |
| 3601 | gctattcatt ctgagggaca gtgggtgaca ctgcctgctc ctctggatac aattaatgtg |
| 3661 | catctgagag ctggatatat tattcctctg cagggacctg gactgacaac aacagagtct |
| 3721 | agacagcagc ctatggctct ggctgtggct ctgacaaagg gaggagaggc tagaggagag |
| 3781 | ctgttttggg atgatggaga gtctctggag gtgctggaga gaggagctta tacacaggtg |
| 3841 | atttttctgg ctagaaataa tacaattgtg aatgagctgg tgagagtgac atctgaggga |
| 3901 | gctggactgc agctgcagaa ggtgacagtg ctgggagtgg ctacagctcc tcagcaggtg |
| 3961 | ctgtctaatg gagtgcctgt gtctaatttt acatattctc ctgatacaaa ggtgctggat |
| 4021 | atttgtgtgt ctctgctgat gggagagcag tttctggtgt cttggtgtta atgatttaaa |
| 4081 | tctcgagccg ggcggagtgt gttagtctct ccagagggag gctggttccc cagggaagca |
| 4141 | gagcctgtgt gcgggcagca gctgtgtgcg ggcctggggg ttgttaagtg caattatttt |
| 4201 | taataaaagg ggcatttgga aaaaaaaaaa aaaggtagca gtcgacagat gaattctgca |
| 4261 | gatctgtggc ttctagctgc ccgggtggca tccctgtgac ccctccccag tgcctctcct |
| 4321 | ggccctggaa gttgccactc cagtgcccac cagccttgtc ctaataaaat taagttgcat |
| 4381 | cattttgtct gactaggtgt ccttctataa tattatgggg tggagggggg tggtatggag |
| 4441 | caaggggcaa gttgggaaga caacctgtag ggcctgcggg gtctattggg aaccaagctg |
| 4501 | gagtgcagtg gcacaatctt ggctcactgc aatctccgcc tcctgggttc aagcgattct |
| 4561 | cctgcctcag cctcccgagt tgttgggatt ccaggcatgc atgaccaggc tcagctaatt |
| 4621 | tttgtttttt tggtagagac ggggtttcac catattggcc aggctggtct ccaactccta |
| 4681 | atctcaggtg atctacccac cttggcctcc caaattgctg ggattacagg cgtgaaccac |
| 4741 | tgctcccttc cctgtccttc tgattttaaa aaaaagagaa atgttctggc acctgcactt |
| 4801 | gcactgggga cagcctattt tgctagtttg ttttgtttcg ttttgttttg atggagagcg |
| 4861 | tatgttgttt aaacgcggcc gcgtagataa gtagcatggc gggttaatca ttaactacaa |
| 4921 | ggaaccccta gtgatggagt tggccactcc ctctctgcgc gctcgctcgc tcactgaggc |
| 4981 | cgggcgacca aaggtcgccc gacgcccggg ctttgcccgg gcggcctcag tgagcgagcg |
| 5041 | agcgcgcagt taattaaggc gccctaggcc gacccttaga ctctgtactc agttctataa |
| 5101 | acgagccatt ggatacgaga tccgtagatt gataagggac acggaatatc cccggacgca |
| 5161 | atagacaccg gtggacagct tggtatcctg agcacagtcg cgcgtccgaa tctagctcta |
| 5221 | ctttagaggc cccggattct gatggtcgta gaccgcagaa ccgattgggg ggatgagatc |
| 5281 | tactagttat cagcacacaa ttgcccatta tacgcgcgta taatggacta ttgtgtgctg |
| 5341 | atatagggat aacagggtaa ttctagagct agcatatgga tccatcgatt tgatgcggta |
| 5401 | ttttctcctt acgcatctgt gcggtatttc acaccgcata cgtcaaagca accatagtac |
| 5461 | gcgccctgta gcggcgcatt aagcgcggcg ggtgtggtgg ttacgcgcag cgtgaccgct |
| 5521 | acacttgcca gcgccctagc gcccgctcct ttcgctttct tcccttcctt tctcgccacg |
| 5581 | ttcgccggct ttccccgtca agctctaaat cgggggctcc ctttagggtt ccgatttagt |
| 5641 | gctttacggc acctcgaccc caaaaaactt gatttgggtg atggttcacg tagtgggcca |
| 5701 | tcgccctgat agacggtttt tcgccctttg acgttggagt ccacgttctt taatagtgga |
| 5761 | ctcttgttcc aaactggaac aacactcaac tctatctcgg gctattcttt tgatttataa |
| 5821 | gggattttgc cgatttcggt ctattggtta aaaaatgagc tgatttaaca aaaatttaac |
| 5881 | gcgaatttta acaaaatatt aacgtttaca attttatggt gcactctcag tacaatctgc |
| 5941 | tctgatgccg catagttaag ccagccccga cacccgccaa cacccgctga cgcgccctga |
| 6001 | cgggcttgtc tgctcccggc atccgcttac agacaagctg tgaccgtctc cgggagctgc |
| 6061 | atgtgtcaga ggttttcacc gtcatcaccg aaacgcgcga gacgaaaggg cctcgtgata |
| 6121 | cgcctatttt tataggttaa tgtcatgata ataatggttt cttagacgtc aggtggcact |
| 6181 | tttcggggaa atgtgcgcgg aacccctatt tgtttatttt tctaaataca ttcaaatatg |
| 6241 | tatccgctca tgagacaata accctgataa atgcttcaat aatattgaaa aaggaagagt |
| 6301 | atgagccata ttcaacggga aacgtcgagg ccgcgattaa attccaacat ggatgctgat |
| 6361 | ttatatgggt ataaatgggc tcgcgataat gtcgggcaat caggtgcgac aatctatcgc |
| 6421 | ttgtatggga agcccgatgc gccagagttg tttctgaaac atggcaaagg tagcgttgcc |
| 6481 | aatgatgtta cagatgagat ggtcagacta aactggctga cggaatttat gcctcttccg |
| 6541 | accatcaagc attttatccg tactcctgat gatgcatggt tactcaccac tgcgatcccc |
| 6601 | ggaaaaacag cattccaggt attagaagaa tatcctgatt caggtgaaaa tattgttgat |
| 6661 | gcgctggcag tgttcctgcg ccggttgcat tcgattcctg tttgtaattg tccttttaac |
| 6721 | agcgatcgcg tatttcgtct cgctcaggcg caatcacgaa tgaataacgg tttggttgat |
| 6781 | gcgagtgatt ttgatgacga gcgtaatggc tggcctgttg aacaagtctg gaaagaaatg |
| 6841 | cataaacttt tgccattctc accggattca gtcgtcactc atggtgattt ctcacttgat |
| 6901 | aaccttattt ttgacgaggg gaaattaata ggttgtattg atgttggacg agtcggaatc |
| 6961 | gcagaccgat accaggatct tgccatccta tggaactgcc tcggtgagtt ttctccttca |
| 7021 | ttacagaaac ggctttttca aaaatatggt attgataatc ctgatatgaa taaattgcag |
| 7081 | tttcatttga tgctcgatga gtttttctaa gcgtataatg gtctagagct agcatatgga |
| 7141 | tccatcgatt ccattatacg cctgtcagac caagtttact catatatact ttagattgat |
| 7201 | ttaaaacttc atttttaatt taaaaggatc taggtgaaga tcctttttga taatctcatg |
| 7261 | accaaaatcc cttaacgtga gttttcgttc cactgagcgt cagaccccgt agaaaagatc |
| 7321 | aaaggatctt cttgagatcc tttttttctg cgcgtaatct gctgcttgca aacaaaaaaa |
| 7381 | ccaccgctac cagcggtggt ttgtttgccg gatcaagagc taccaactct ttttccgaag |
| 7441 | gtaactggct tcagcagagc gcagatacca aatactgttc ttctagtgta gccgtagtta |
| 7501 | ggccaccact tcaagaactc tgtagcaccg cctacatacc tcgctctgct aatcctgtta |
| 7561 | ccagtggctg ctgccagtgg cgataagtcg tgtcttaccg ggttggactc aagacgatag |
| 7621 | ttaccggata aggcgcagcg gtcgggctga acggggggtt cgtgcacaca gcccagcttg |
| 7681 | gagcgaacga cctacaccga actgagatac ctacagcgtg agctatgaga aagcgccacg |
| 7741 | cttcccgaag ggagaaaggc ggacaggtat ccggtaagcg gcagggtcgg aacaggagag |
| 7801 | cgcacgaggg agcttccagg gggaaacgcc tggtatcttt atagtcctgt cgggtttcgc |
| 7861 | cacctctgac ttgagcgtcg atttttgtga tgctcgtcag gggggcggag cctatggaaa |
| 7921 | aacgccagca acgcggcctt tttacggttc ctggcctttt gctggccttt tgctcacatg |
| 7981 | t |

| pP086_seq3-201Ig_5-56 deletion (SEQ ID NO: 491) | |
|---|---|
| (SEQ ID NO: 491) | |
| 1 | tctagagcta gcatatggat ccatcgattt agggataaca gggtaattat cagcacacaa |
| 61 | ttgcccatta tacgcgcgta taatggacta ttgtgtgctg atatctgtac acttaagggc |
| 121 | tagatcttag cttacgtcac tagagggtcc acgtttagtt tttaagatcc attgatctcc |
| 181 | taaacgctgc aagattcgca acctggtata cttagcctag gcgctaggtc ctagtgcagc |
| 241 | gggacttttt ttctaaagtc gttgagagga ggagtcgtca gaccagatag ctttgatgtc |
| 301 | ctgatcggaa ggatcgttgg cccccctgca ggcagctgtt aattaactgc gcgctcgctc |
| 361 | gctcactgag gccgcccggg caaagcccgg gcgtcgggcg acctttggtc gcccggcctc |
| 421 | agtgagcgag cgagcgcgca gagagggagt ggccaactcc atcactaggg gttccttgta |
| 481 | gttaatgatt aacccgccat gctacttatc tacggcgcgc cacgcgttct aggagttaat |
| 541 | ttttaaaaag cagtcaaaag tccaagtgcc cttgcgagca tttactctct ctgtttgctc |
| 601 | tggttaataa tctcaggagc acaaacattc cttactagtt ctaggagtta atttttaaaa |
| 661 | agcagtcaaa agtccaagtg cccttgcgag catttactct ctctgtttgc tctggttaat |
| 721 | aatctcagga gcacaaacat tccttactag ttctagagcg gccgccagtg tgctggaatt |
| 781 | cggctttttt agggctggaa gctacctttg acatcatttc ctctgcgaat gcatgtataa |
| 841 | tttctacaga acctattaga aaggatcacc cagcctctgc ttttgtacaa ctttccctta |
| 901 | aaaaactgcc aatcccactg ctgtttggcc caatagtgag aactttttcc tgctgcctct |
| 961 | tggtgctttt gcctatggcc cctattctgc ctgctgaaga cactcttgcc agcatggact |
| 1021 | taaacccctc cagctctgac aatcctcttt ctcttttgtt ttacatgaag ggtctggcag |
| 1081 | ccaaagcaat cactcaaagt tcaaacctta tcattttttg ctttgttcct cttggccttg |
| 1141 | gttttgtaca tcagctttga aaataccatc ccagggttaa tgctggggtt aatttataac |
| 1201 | tgagagtgct ctagttctgc aatacaggac atgctataaa aatggaaaga tgttgctttc |
| 1261 | tgagagatca ggtggattct tgggcatttg cttaagcttg gtaccgccac catgggggtg |
| 1321 | agggagtttg ggctgagctg ggtctttctg gtggccctgc tgaagggagt ccagtgtgag |
| 1381 | cagcagggag cttctagacc tggacctaga gatgctcagg ctcatcctgg aagacctaga |
| 1441 | gctgtgccta cacagtgtga tgtgcctcct aattctagat ttgattgtgc tcctgataag |
| 1501 | gctataacac aggagcagtg tgaggctaga ggatgttgtt atattcctgc taagcaggga |
| 1561 | ctgcagggag ctcagatggg acagccttgg tgtttttttc ctccttctta tccttcttat |
| 1621 | aagctggaga atctgtcttc ttctgagatg ggatatacag ctacactgac aagaacaaca |
| 1681 | cctacatttt ttcctaagga tattctgaca ctgagactgg atgtgatgat ggagacagag |
| 1741 | aatagactgc attttacaat taaggatcct gctaatagaa gatatgaggt gcctctagag |
| 1801 | acacctagag tgcattctag agctccttct cctctgtatt ctgtggagtt ttctgaggag |
| 1861 | ccttttggag tgattgtgca tagacagctg gatggaagag tgctgctgaa tacaacagtg |
| 1921 | gctcctctgt tttttgctga tcagtttctg cagctgtcta catctctgcc ttctcagtat |
| 1981 | attacaggac tggctgagca tctgtctcct ctgatgctgt ctacatcttg gacaagaatt |
| 2041 | acactgtgga atagagatct ggctcctaca cctggagcta atctgtatgg atctcatcct |
| 2101 | ttttatctgg ctctggagga tggaggatct gctcatggag tgtttctgct gaattctaat |
| 2161 | gctatggatg tagtgctgca gccttctcct gctctgtctt ggagatctac aggaggaatt |
| 2221 | ctggatgtgt atatttttct aggacctgag cctaagtctg tggtgcagca gtatctggat |
| 2281 | gtagtgggat atccttttat gcctccttat tggggactgg gatttcatct gtgtagatgg |
| 2341 | ggatattctt ctacagctat tacaagacag gtggttgaga atatgacaag agctcatttt |
| 2401 | cctctggatg tgcagtggaa tgatctagat tatatggatt ctagaagaga ttttacattt |
| 2461 | aataaggatg gatttagaga ttttcctgct atggtgcagg agctgcatca gggaggaaga |
| 2521 | agatatatga tgattgtgga tcctgctatt tcttcttctg gacctgctgg atcttataga |
| 2581 | ccttatgatg agggactgag aagaggagtg tttattacaa atgagacagg acagcctctg |
| 2641 | attggaaagg tgtggcctgg atctacagct tttcctgatt ttacaaatcc tacagctctg |
| 2701 | gcttggtggg aggatatggt ggctgagttt catgatcagg tgccttttga tggaatgtgg |
| 2761 | attgatatga atgagccttc taattttata agaggatctg aggatggatg tcctaataat |
| 2821 | gagctagaga atcctcctta tgtgcctgga gtagtgggag gaacactgca ggctgctaca |
| 2881 | atttgtgctt cttctcatca gtttctgtct acacattata atctgcataa tctgtatgga |
| 2941 | ctgacagagg ctattgcttc tcatagagct ctggtgaagg ctagaggaac aagacctttt |
| 3001 | gtgatttcta gatctacatt tgctggacat ggaagatatg ctggacattg gacaggagat |
| 3061 | gtatggtctt cttgggagca gctagcttct tctgtgcctg agattctgca gtttaatctg |
| 3121 | ctaggagtgc ctctggtggg agctgatgtg tgtggatttc tgggaaatac atctgaggag |
| 3181 | ctgtgtgtga gatggacaca gctgggagct ttttatcctt ttatgagaaa tcataattct |
| 3241 | ctgctgtctc tgcctcagga gccttattct ttttctgagc ctgctcagca ggctatgaga |
| 3301 | aaggctctga cactgagata tgctctgctg cctcatctgt atacactgtt tcatcaggct |
| 3361 | catgtggctg gagagacagt agctagacct ctgtttctgg agtttcctaa ggattcttct |
| 3421 | acatggacag ttgatcatca gctgctgtgg ggagaggctc tgctgattac acctgtgctg |
| 3481 | caggctggaa aggctgaggt gacaggatat tttcctctgg gaacatggta tgatctgcag |
| 3541 | acagtgccta tagaggctct gggatctctg cctcctcctc ctgctgctcc tagagagcct |
| 3601 | gctattcatt ctgagggaca gtgggtgaca ctgcctgctc ctctggatac aattaatgtg |
| 3661 | catctgagag ctggatatat tattcctctg cagggacctg gactgacaac aacagagtct |
| 3721 | agacagcagc ctatggctct ggctgtggct ctgacaaagg gaggagaggc tagaggagag |
| 3781 | ctgttttggg atgatggaga gtctctggag gtgctggaga gaggagctta tacacaggtg |
| 3841 | atttttctgg ctagaaataa tacaattgtg aatgagctgg tgagagtgac atctgaggga |
| 3901 | gctggactgc agctgcagaa ggtgacagtg ctgggagtgg ctacagctcc tcagcaggtg |
| 3961 | ctgtctaatg gagtgcctgt gtctaatttt acatattctc ctgatacaaa ggtgctggat |
| 4021 | atttgtgtgt ctctgctgat gggagagcag tttctggtgt cttggtgtta atgatttaaa |
| 4081 | tctcgagccg ggcggagtgt gttagtctct ccagagggag gctggttccc cagggaagca |
| 4141 | gagcctgtgt gcgggcagca gctgtgtgcg ggcctggggg ttgttaagtg caattatttt |
| 4201 | taataaaagg ggcatttgga aaaaaaaaaa aaaggtagca gtcgacagat gaattctgca |
| 4261 | gatctgtggc ttctagctgc ccgggtggca tccctgtgac ccctccccag tgcctctcct |
| 4321 | ggccctggaa gttgccactc cagtgcccac cagccttgtc ctaataaaat taagttgcat |
| 4381 | cattttgtct gactaggtgt ccttctataa tattatgggg tggagggggg tggtatggag |
| 4441 | caaggggcaa gttgggaaga caacctgtag ggcctgcggg gtctattggg aaccaagctg |
| 4501 | gagtgcagtg gcacaatctt ggctcactgc aatctccgcc tcctgggttc aagcgattct |
| 4561 | cctgcctcag cctcccgagt tgttgggatt ccaggcatgc atgaccaggc tcagctaatt |
| 4621 | tttgtttttt tggtagagac ggggtttcac catattggcc aggctggtct ccaactccta |
| 4681 | atctcaggtg atctacccac cttggcctcc caaattgctg ggattacagg cgtgaaccac |
| 4741 | tgctcccttc cctgtccttc tgattttaaa aaaaagagaa atgttctggc acctgcactt |
| 4801 | gcactgggga cagcctattt tgctagtttg ttttgtttcg ttttgttttg atggagagcg |
| 4861 | tatgttgttt aaacgcggcc gcgtagataa gtagcatggc gggttaatca ttaactacaa |
| 4921 | ggaaccccta gtgatggagt tggccactcc ctctctgcgc gctcgctcgc tcactgaggc |
| 4981 | cgggcgacca aaggtcgccc gacgcccggg ctttgcccgg gcggcctcag tgagcgagcg |
| 5041 | agcgcgcagt taattaaggc gccctaggcc gacccttaga ctctgtactc agttctataa |
| 5101 | acgagccatt ggatacgaga tccgtagatt gataagggac acggaatatc cccggacgca |
| 5161 | atagacaccg gtggacagct tggtatcctg agcacagtcg cgcgtccgaa tctagctcta |
| 5221 | ctttagaggc cccggattct gatggtcgta gaccgcagaa ccgattgggg ggatgagatc |
| 5281 | tactagttat cagcacacaa ttgcccatta tacgcgcgta taatggacta ttgtgtgctg |
| 5341 | atatagggat aacagggtaa ttctagagct agcatatgga tccatcgatt tgatgcggta |
| 5401 | ttttctcctt acgcatctgt gcggtatttc acaccgcata cgtcaaagca accatagtac |
| 5461 | gcgccctgta gcggcgcatt aagcgcggcg ggtgtggtgg ttacgcgcag cgtgaccgct |
| 5521 | acacttgcca gcgccctagc gcccgctcct ttcgctttct tcccttcctt tctcgccacg |
| 5581 | ttcgccggct ttccccgtca agctctaaat cgggggctcc ctttagggtt ccgatttagt |
| 5641 | gctttacggc acctcgaccc caaaaaactt gatttgggtg atggttcacg tagtgggcca |
| 5701 | tcgccctgat agacggtttt tcgccctttg acgttggagt ccacgttctt taatagtgga |
| 5761 | ctcttgttcc aaactggaac aacactcaac tctatctcgg gctattcttt tgatttataa |
| 5821 | gggattttgc cgatttcggt ctattggtta aaaaatgagc tgatttaaca aaaatttaac |
| 5881 | gcgaatttta acaaaatatt aacgtttaca attttatggt gcactctcag tacaatctgc |
| 5941 | tctgatgccg catagttaag ccagccccga cacccgccaa cacccgctga cgcgccctga |
| 6001 | cgggcttgtc tgctcccggc atccgcttac agacaagctg tgaccgtctc cgggagctgc |
| 6061 | atgtgtcaga ggttttcacc gtcatcaccg aaacgcgcga gacgaaaggg cctcgtgata |
| 6121 | cgcctatttt tataggttaa tgtcatgata ataatggttt cttagacgtc aggtggcact |
| 6181 | tttcggggaa atgtgcgcgg aacccctatt tgtttatttt tctaaataca ttcaaatatg |
| 6241 | tatccgctca tgagacaata accctgataa atgcttcaat aatattgaaa aaggaagagt |
| 6301 | atgagccata ttcaacggga aacgtcgagg ccgcgattaa attccaacat ggatgctgat |
| 6361 | ttatatgggt ataaatgggc tcgcgataat gtcgggcaat caggtgcgac aatctatcgc |
| 6421 | ttgtatggga agcccgatgc gccagagttg tttctgaaac atggcaaagg tagcgttgcc |
| 6481 | aatgatgtta cagatgagat ggtcagacta aactggctga cggaatttat gcctcttccg |
| 6541 | accatcaagc attttatccg tactcctgat gatgcatggt tactcaccac tgcgatcccc |
| 6601 | ggaaaaacag cattccaggt attagaagaa tatcctgatt caggtgaaaa tattgttgat |
| 6661 | gcgctggcag tgttcctgcg ccggttgcat tcgattcctg tttgtaattg tccttttaac |
| 6721 | agcgatcgcg tatttcgtct cgctcaggcg caatcacgaa tgaataacgg tttggttgat |
| 6781 | gcgagtgatt ttgatgacga gcgtaatggc tggcctgttg aacaagtctg gaaagaaatg |
| 6841 | cataaacttt tgccattctc accggattca gtcgtcactc atggtgattt ctcacttgat |
| 6901 | aaccttattt ttgacgaggg gaaattaata ggttgtattg atgttggacg agtcggaatc |
| 6961 | gcagaccgat accaggatct tgccatccta tggaactgcc tcggtgagtt ttctccttca |
| 7021 | ttacagaaac ggctttttca aaaatatggt attgataatc ctgatatgaa taaattgcag |
| 7081 | tttcatttga tgctcgatga gtttttctaa gcgtataatg gtctagagct agcatatgga |
| 7141 | tccatcgatt ccattatacg cctgtcagac caagtttact catatatact ttagattgat |
| 7201 | ttaaaacttc atttttaatt taaaaggatc taggtgaaga tcctttttga taatctcatg |
| 7261 | accaaaatcc cttaacgtga gttttcgttc cactgagcgt cagaccccgt agaaaagatc |
| 7321 | aaaggatctt cttgagatcc tttttttctg cgcgtaatct gctgcttgca aacaaaaaaa |
| 7381 | ccaccgctac cagcggtggt ttgtttgccg gatcaagagc taccaactct ttttccgaag |
| 7441 | gtaactggct tcagcagagc gcagatacca aatactgttc ttctagtgta gccgtagtta |
| 7501 | ggccaccact tcaagaactc tgtagcaccg cctacatacc tcgctctgct aatcctgtta |
| 7561 | ccagtggctg ctgccagtgg cgataagtcg tgtcttaccg ggttggactc aagacgatag |
| 7621 | ttaccggata aggcgcagcg gtcgggctga acggggggtt cgtgcacaca gcccagcttg |
| 7681 | gagcgaacga cctacaccga actgagatac ctacagcgtg agctatgaga aagcgccacg |
| 7741 | cttcccgaag ggagaaaggc ggacaggtat ccggtaagcg gcagggtcgg aacaggagag |
| 7801 | cgcacgaggg agcttccagg gggaaacgcc tggtatcttt atagtcctgt cgggtttcgc |
| 7861 | cacctctgac ttgagcgtcg atttttgtga tgctcgtcag gggggcggag cctatggaaa |
| 7921 | aacgccagca acgcggcctt tttacggttc ctggcctttt gctggccttt tgctcacatg |
| 7981 | t |

| pP087_seq100-IL2wt_11-56 deletion (SEQ ID NO: 492) | |
|---|---|
| (SEQ ID NO: 492) | |
| 1 | tctagagcta gcatatggat ccatcgattt agggataaca gggtaattat cagcacacaa |
| 61 | ttgcccatta tacgcgcgta taatggacta ttgtgtgctg atatctgtac acttaagggc |
| 121 | tagatcttag cttacgtcac tagagggtcc acgtttagtt tttaagatcc attgatctcc |
| 181 | taaacgctgc aagattcgca acctggtata cttagcctag gcgctaggtc ctagtgcagc |
| 241 | gggacttttt ttctaaagtc gttgagagga ggagtcgtca gaccagatag ctttgatgtc |
| 301 | ctgatcggaa ggatcgttgg cccccctgca ggcagctgtt aattaactgc gcgctcgctc |
| 361 | gctcactgag gccgcccggg caaagcccgg gcgtcgggcg acctttggtc gcccggcctc |
| 421 | agtgagcgag cgagcgcgca gagagggagt ggccaactcc atcactaggg gttccttgta |
| 481 | gttaatgatt aacccgccat gctacttatc tacggcgcgc cacgcgttct aggagttaat |
| 541 | ttttaaaaag cagtcaaaag tccaagtgcc cttgcgagca tttactctct ctgtttgctc |
| 601 | tggttaataa tctcaggagc acaaacattc cttactagtt ctaggagtta atttttaaaa |
| 661 | agcagtcaaa agtccaagtg cccttgcgag catttactct ctctgtttgc tctggttaat |
| 721 | aatctcagga gcacaaacat tccttactag ttctagagcg gccgccagtg tgctggaatt |
| 781 | cggctttttt agggctggaa gctacctttg acatcatttc ctctgcgaat gcatgtataa |
| 841 | tttctacaga acctattaga aaggatcacc cagcctctgc ttttgtacaa ctttccctta |
| 901 | aaaaactgcc aatcccactg ctgtttggcc caatagtgag aactttttcc tgctgcctct |
| 961 | tggtgctttt gcctatggcc cctattctgc ctgctgaaga cactcttgcc agcatggact |
| 1021 | taaacccctc cagctctgac aatcctcttt ctcttttgtt ttacatgaag ggtctggcag |
| 1081 | ccaaagcaat cactcaaagt tcaaacctta tcattttttg ctttgttcct cttggccttg |
| 1141 | gttttgtaca tcagctttga aaataccatc ccagggttaa tgctggggtt aatttataac |
| 1201 | tgagagtgct ctagttctgc aatacaggac atgctataaa aatggaaaga tgttgctttc |
| 1261 | tgagagatca ggtggattct tgggcatttg cttaagcttg gtaccgccac catgggggtg |
| 1321 | aggcaccctc cctgttctca ctacagaatg cagctgctgt cctgcattgc actgtcactg |
| 1381 | gcactggtta ccaactcaca gcagggggcc agcaggccag gccccaggga tgcccaggcc |
| 1441 | caccctggca gacccagggc tgtgcccacc cagtgtgatg tgcctcccaa cagcagattt |
| 1501 | gactgtgccc ctgataaggc cattactcag gaacagtgtg aggccagggg ctgctgctat |
| 1561 | atccctgcca agcagggcct gcagggggcc cagatggggc agccctggtg cttcttcccc |
| 1621 | cccagctacc cctcttataa gctggagaat ctgagcagct ctgaaatggg ctacactgcc |
| 1681 | actctgacca ggactacccc caccttcttc cccaaggata ttctgactct gaggctggat |
| 1741 | gtgatgatgg agactgagaa caggctgcac ttcaccatca aggaccctgc caacaggagg |
| 1801 | tatgaggtgc ccctggaaac tcccagggtg cactctaggg cccccagccc cctgtattct |
| 1861 | gtggagttct ctgaggagcc ttttggggtc attgtccaca ggcagctgga tggcagggtg |
| 1921 | ctgctgaata ctactgtggc ccctctgttc tttgctgacc agttcctgca gctgagcacc |
| 1981 | agcctgcctt ctcagtacat tactggcctg gctgagcatc tgagccccct gatgctgagc |
| 2041 | acctcttgga ccagaatcac cctgtggaac agggacctgg ctcccactcc tggggccaac |
| 2101 | ctgtatggca gccacccctt ctacctggcc ctggaggatg ggggctctgc ccatggggtg |
| 2161 | ttcctgctga acagcaatgc catggatgtg gtgctgcagc cctctcctgc cctgtcttgg |
| 2221 | agatctactg ggggcatcct ggatgtgtat atcttcctgg ggcctgagcc caagtctgtg |
| 2281 | gtgcagcagt acctggatgt ggtgggctac cccttcatgc ccccctactg gggcctgggc |
| 2341 | ttccacctgt gcaggtgggg ctactcttct actgctatca ccaggcaggt ggtggagaac |
| 2401 | atgaccaggg ctcacttccc tctggatgtg cagtggaatg acctggacta catggactct |
| 2461 | aggagagact tcacttttaa taaggatggg ttcagggact ttcctgccat ggtgcaggag |
| 2521 | ctgcatcagg ggggcaggag atatatgatg attgtggacc ctgctatttc tagctctggc |
| 2581 | cctgctggca gctataggcc ctatgatgag gggctgagga ggggggtgtt catcactaat |
| 2641 | gagactggcc agcccctgat tggcaaggtg tggcctggct ctactgcctt ccctgatttc |
| 2701 | accaacccca ctgccctggc ctggtgggag gatatggtgg ctgagtttca tgaccaggtg |
| 2761 | ccctttgatg gcatgtggat tgacatgaat gagcccagca actttatcag gggctctgaa |
| 2821 | gatggctgcc ccaacaatga gctggagaac cccccctatg tgcctggggt ggtggggggc |
| 2881 | accctgcagg ctgccaccat ctgtgccagc agccaccagt tcctgagcac ccactataac |
| 2941 | ctgcacaacc tgtatggcct gactgaggcc attgcctctc acagggccct ggtgaaggct |
| 3001 | agggggacta ggccctttgt gatcagcagg tctacttttg ctggccatgg caggtatgct |
| 3061 | gggcactgga ctggggatgt gtggtctagc tgggagcagc tggccagctc tgtgcctgag |
| 3121 | atcctgcagt ttaatctgct gggggtgccc ctggtggggg ctgatgtgtg tggcttcctg |
| 3181 | ggcaatacct ctgaggagct gtgtgtgagg tggactcagc tgggggcttt ctaccccttc |
| 3241 | atgagaaacc acaactctct gctgagcctg ccccaggagc cctattcttt ttctgagcct |
| 3301 | gcccagcagg ctatgaggaa ggccctgact ctgaggtatg ccctgctgcc ccacctgtat |
| 3361 | accctgttcc atcaggccca tgtggctggg gagactgtgg ccagacctct gttcctggag |
| 3421 | ttccccaagg atagctctac ttggactgtg gaccaccagc tgctgtgggg ggaggctctg |
| 3481 | ctgatcaccc ctgtgctgca ggctgggaag gctgaggtga ctggctattt ccccctgggc |
| 3541 | acctggtatg atctgcagac tgtgcccatt gaggccctgg gctctctgcc ccctccccct |
| 3601 | gctgccccca gggagcctgc catccactct gagggccagt gggtgaccct gcctgcccct |
| 3661 | ctggacacta tcaatgtgca cctgagggct ggctacatca tccccctgca gggccctggc |
| 3721 | ctgactacca ctgagtctag gcagcagccc atggccctgg ctgtggctct gaccaagggg |
| 3781 | ggggaggcca ggggggagct gttctgggat gatggggagt ctctggaggt gctggagagg |
| 3841 | ggggcctaca cccaggtgat cttcctggct aggaataaca ccattgtcaa tgagctggtg |
| 3901 | agggtgacct ctgagggggc tggcctgcag ctgcagaagg tgactgtgct gggggtggct |
| 3961 | actgcccccc agcaggtgct gagcaatggg gtgcctgtga gcaacttcac ctatagccct |
| 4021 | gacaccaagg tgctggacat ctgtgtgagc ctgctgatgg gggagcagtt cctggtgagc |
| 4081 | tggtgctaat ttaaatctcg agccgggcgg agtgtgttag tctctccaga gggaggctgg |
| 4141 | ttccccaggg aagcagagcc tgtgtgcggg cagcagctgt gtgcgggcct gggggttgtt |
| 4201 | aagtgcaatt atttttaata aaaggggcat ttggaaaaaa aaaaaaaagg tagcagtcga |
| 4261 | cagatgaatt ctgcagatct gtggcttcta gctgcccggg tggcatccct gtgacccctc |
| 4321 | cccagtgcct ctcctggccc tggaagttgc cactccagtg cccaccagcc ttgtcctaat |
| 4381 | aaaattaagt tgcatcattt tgtctgacta ggtgtccttc tataatatta tggggtggag |
| 4441 | gggggtggta tggagcaagg ggcaagttgg gaagacaacc tgtagggcct gcggggtcta |
| 4501 | ttgggaacca agctggagtg cagtggcaca atcttggctc actgcaatct ccgcctcctg |
| 4561 | ggttcaagcg attctcctgc ctcagcctcc cgagttgttg ggattccagg catgcatgac |
| 4621 | caggctcagc taatttttgt ttttttggta gagacggggt ttcaccatat tggccaggct |
| 4681 | ggtctccaac tcctaatctc aggtgatcta cccaccttgg cctcccaaat tgctgggatt |
| 4741 | acaggcgtga accactgctc ccttccctgt ccttctgatt ttaaaaaaaa gagaaatgtt |
| 4801 | ctggcacctg cacttgcact ggggacagcc tattttgcta gtttgttttg tttcgttttg |
| 4861 | ttttgatgga gagcgtatgt tgtttaaacg cggccgcgta gataagtagc atggcgggtt |
| 4921 | aatcattaac tacaaggaac ccctagtgat ggagttggcc actccctctc tgcgcgctcg |
| 4981 | ctcgctcact gaggccgggc gaccaaaggt cgcccgacgc ccgggctttg cccgggcggc |
| 5041 | ctcagtgagc gagcgagcgc gcagttaatt aaggcgccct aggccgaccc ttagactctg |
| 5101 | tactcagttc tataaacgag ccattggata cgagatccgt agattgataa gggacacgga |
| 5161 | atatccccgg acgcaataga caccggtgga cagcttggta tcctgagcac agtcgcgcgt |
| 5221 | ccgaatctag ctctacttta gaggccccgg attctgatgg tcgtagaccg cagaaccgat |
| 5281 | tggggggatg agatctacta gttatcagca cacaattgcc cattatacgc gcgtataatg |
| 5341 | gactattgtg tgctgatata gggataacag ggtaattcta gagctagcat atggatccat |
| 5401 | cgatttgatg cggtattttc tccttacgca tctgtgcggt atttcacacc gcatacgtca |
| 5461 | aagcaaccat agtacgcgcc ctgtagcggc gcattaagcg cggcgggtgt ggtggttacg |
| 5521 | cgcagcgtga ccgctacact tgccagcgcc ctagcgcccg ctcctttcgc tttcttccct |
| 5581 | tcctttctcg ccacgttcgc cggctttccc cgtcaagctc taaatcgggg gctcccttta |
| 5641 | gggttccgat ttagtgcttt acggcacctc gaccccaaaa aacttgattt gggtgatggt |
| 5701 | tcacgtagtg ggccatcgcc ctgatagacg gtttttcgcc ctttgacgtt ggagtccacg |
| 5761 | ttctttaata gtggactctt gttccaaact ggaacaacac tcaactctat ctcgggctat |
| 5821 | tcttttgatt tataagggat tttgccgatt tcggtctatt ggttaaaaaa tgagctgatt |
| 5881 | taacaaaaat ttaacgcgaa ttttaacaaa atattaacgt ttacaatttt atggtgcact |
| 5941 | ctcagtacaa tctgctctga tgccgcatag ttaagccagc cccgacaccc gccaacaccc |
| 6001 | gctgacgcgc cctgacgggc ttgtctgctc ccggcatccg cttacagaca agctgtgacc |
| 6061 | gtctccggga gctgcatgtg tcagaggttt tcaccgtcat caccgaaacg cgcgagacga |
| 6121 | aagggcctcg tgatacgcct atttttatag gttaatgtca tgataataat ggtttcttag |
| 6181 | acgtcaggtg gcacttttcg gggaaatgtg cgcggaaccc ctatttgttt atttttctaa |
| 6241 | atacattcaa atatgtatcc gctcatgaga caataaccct gataaatgct tcaataatat |
| 6301 | tgaaaaagga agagtatgag ccatattcaa cgggaaacgt cgaggccgcg attaaattcc |
| 6361 | aacatggatg ctgatttata tgggtataaa tgggctcgcg ataatgtcgg gcaatcaggt |
| 6421 | gcgacaatct atcgcttgta tgggaagccc gatgcgccag agttgtttct gaaacatggc |
| 6481 | aaaggtagcg ttgccaatga tgttacagat gagatggtca gactaaactg gctgacggaa |
| 6541 | tttatgcctc ttccgaccat caagcatttt atccgtactc ctgatgatgc atggttactc |
| 6601 | accactgcga tccccggaaa aacagcattc caggtattag aagaatatcc tgattcaggt |
| 6661 | gaaaatattg ttgatgcgct ggcagtgttc ctgcgccggt tgcattcgat tcctgtttgt |
| 6721 | aattgtcctt ttaacagcga tcgcgtattt cgtctcgctc aggcgcaatc acgaatgaat |
| 6781 | aacggtttgg ttgatgcgag tgattttgat gacgagcgta atggctggcc tgttgaacaa |
| 6841 | gtctggaaag aaatgcataa acttttgcca ttctcaccgg attcagtcgt cactcatggt |
| 6901 | gatttctcac ttgataacct tatttttgac gaggggaaat taataggttg tattgatgtt |
| 6961 | ggacgagtcg gaatcgcaga ccgataccag gatcttgcca tcctatggaa ctgcctcggt |
| 7021 | gagttttctc cttcattaca gaaacggctt tttcaaaaat atggtattga taatcctgat |
| 7081 | atgaataaat tgcagtttca tttgatgctc gatgagtttt tctaagcgta taatggtcta |
| 7141 | gagctagcat atggatccat cgattccatt atacgcctgt cagaccaagt ttactcatat |
| 7201 | atactttaga ttgatttaaa acttcatttt taatttaaaa ggatctaggt gaagatcctt |
| 7261 | tttgataatc tcatgaccaa aatcccttaa cgtgagtttt cgttccactg agcgtcagac |
| 7321 | cccgtagaaa agatcaaagg atcttcttga gatccttttt ttctgcgcgt aatctgctgc |
| 7381 | ttgcaaacaa aaaaaccacc gctaccagcg gtggtttgtt tgccggatca agagctacca |
| 7441 | actctttttc cgaaggtaac tggcttcagc agagcgcaga taccaaatac tgttcttcta |
| 7501 | gtgtagccgt agttaggcca ccacttcaag aactctgtag caccgcctac atacctcgct |
| 7561 | ctgctaatcc tgttaccagt ggctgctgcc agtggcgata agtcgtgtct taccgggttg |
| 7621 | gactcaagac gatagttacc ggataaggcg cagcggtcgg gctgaacggg gggttcgtgc |
| 7681 | acacagccca gcttggagcg aacgacctac accgaactga gatacctaca gcgtgagcta |
| 7741 | tgagaaagcg ccacgcttcc cgaagggaga aaggcggaca ggtatccggt aagcggcagg |
| 7801 | gtcggaacag gagagcgcac gagggagctt ccagggggaa acgcctggta tctttatagt |
| 7861 | cctgtcgggt ttcgccacct ctgacttgag cgtcgatttt tgtgatgctc gtcagggggg |
| 7921 | cggagcctat ggaaaaacgc cagcaacgcg gcctttttac ggttcctggc cttttgctgg |
| 7981 | ccttttgctc acatgt |

| pP088_seq100-201Ig_11-56 deletion (SEQ ID NO: 493) | |
|---|---|
| (SEQ ID NO: 493) | |
| 1 | tctagagcta gcatatggat ccatcgattt agggataaca gggtaattat cagcacacaa |
| 61 | ttgcccatta tacgcgcgta taatggacta ttgtgtgctg atatctgtac acttaagggc |
| 121 | tagatcttag cttacgtcac tagagggtcc acgtttagtt tttaagatcc attgatctcc |
| 181 | taaacgctgc aagattcgca acctggtata cttagcctag gcgctaggtc ctagtgcagc |
| 241 | gggacttttt ttctaaagtc gttgagagga ggagtcgtca gaccagatag ctttgatgtc |
| 301 | ctgatcggaa ggatcgttgg cccccctgca ggcagctgtt aattaactgc gcgctcgctc |
| 361 | gctcactgag gccgcccggg caaagcccgg gcgtcgggcg acctttggtc gcccggcctc |
| 421 | agtgagcgag cgagcgcgca gagagggagt ggccaactcc atcactaggg gttccttgta |
| 481 | gttaatgatt aacccgccat gctacttatc tacggcgcgc cacgcgttct aggagttaat |
| 541 | ttttaaaaag cagtcaaaag tccaagtgcc cttgcgagca tttactctct ctgtttgctc |
| 601 | tggttaataa tctcaggagc acaaacattc cttactagtt ctaggagtta atttttaaaa |
| 661 | agcagtcaaa agtccaagtg cccttgcgag catttactct ctctgtttgc tctggttaat |
| 721 | aatctcagga gcacaaacat tccttactag ttctagagcg gccgccagtg tgctggaatt |
| 781 | cggctttttt agggctggaa gctacctttg acatcatttc ctctgcgaat gcatgtataa |
| 841 | tttctacaga acctattaga aaggatcacc cagcctctgc ttttgtacaa ctttccctta |
| 901 | aaaaactgcc aatcccactg ctgtttggcc caatagtgag aactttttcc tgctgcctct |
| 961 | tggtgctttt gcctatggcc cctattctgc ctgctgaaga cactcttgcc agcatggact |
| 1021 | taaacccctc cagctctgac aatcctcttt ctcttttgtt ttacatgaag ggtctggcag |
| 1081 | ccaaagcaat cactcaaagt tcaaacctta tcattttttg ctttgttcct cttggccttg |
| 1141 | gttttgtaca tcagctttga aaataccatc ccagggttaa tgctggggtt aatttataac |
| 1201 | tgagagtgct ctagttctgc aatacaggac atgctataaa aatggaaaga tgttgctttc |
| 1261 | tgagagatca ggtggattct tgggcatttg cttaagcttg gtaccgccac catgggggtg |
| 1321 | aggcaccctc cctgttctca tgagtttggg ctgagctggg tctttctggt ggccctgctg |
| 1381 | aagggagtcc agtgtgagca gcagggggcc agcaggccag gccccaggga tgcccaggcc |
| 1441 | caccctggca gacccagggc tgtgcccacc cagtgtgatg tgcctcccaa cagcagattt |
| 1501 | gactgtgccc ctgataaggc cattactcag gaacagtgtg aggccagggg ctgctgctat |
| 1561 | atccctgcca agcagggcct gcagggggcc cagatggggc agccctggtg cttcttcccc |
| 1621 | cccagctacc cctcttataa gctggagaat ctgagcagct ctgaaatggg ctacactgcc |
| 1681 | actctgacca ggactacccc caccttcttc cccaaggata ttctgactct gaggctggat |
| 1741 | gtgatgatgg agactgagaa caggctgcac ttcaccatca aggaccctgc caacaggagg |
| 1801 | tatgaggtgc ccctggaaac tcccagggtg cactctaggg cccccagccc cctgtattct |
| 1861 | gtggagttct ctgaggagcc ttttggggtc attgtccaca ggcagctgga tggcagggtg |
| 1921 | ctgctgaata ctactgtggc ccctctgttc tttgctgacc agttcctgca gctgagcacc |
| 1981 | agcctgcctt ctcagtacat tactggcctg gctgagcatc tgagccccct gatgctgagc |
| 2041 | acctcttgga ccagaatcac cctgtggaac agggacctgg ctcccactcc tggggccaac |
| 2101 | ctgtatggca gccacccctt ctacctggcc ctggaggatg ggggctctgc ccatggggtg |
| 2161 | ttcctgctga acagcaatgc catggatgtg gtgctgcagc cctctcctgc cctgtcttgg |
| 2221 | agatctactg ggggcatcct ggatgtgtat atcttcctgg ggcctgagcc caagtctgtg |
| 2281 | gtgcagcagt acctggatgt ggtgggctac cccttcatgc ccccctactg gggcctgggc |
| 2341 | ttccacctgt gcaggtgggg ctactcttct actgctatca ccaggcaggt ggtggagaac |
| 2401 | atgaccaggg ctcacttccc tctggatgtg cagtggaatg acctggacta catggactct |
| 2461 | aggagagact tcacttttaa taaggatggg ttcagggact ttcctgccat ggtgcaggag |
| 2521 | ctgcatcagg ggggcaggag atatatgatg attgtggacc ctgctatttc tagctctggc |
| 2581 | cctgctggca gctataggcc ctatgatgag gggctgagga ggggggtgtt catcactaat |
| 2641 | gagactggcc agcccctgat tggcaaggtg tggcctggct ctactgcctt ccctgatttc |
| 2701 | accaacccca ctgccctggc ctggtgggag gatatggtgg ctgagtttca tgaccaggtg |
| 2761 | ccctttgatg gcatgtggat tgacatgaat gagcccagca actttatcag gggctctgaa |
| 2821 | gatggctgcc ccaacaatga gctggagaac cccccctatg tgcctggggt ggtggggggc |
| 2881 | accctgcagg ctgccaccat ctgtgccagc agccaccagt tcctgagcac ccactataac |
| 2941 | ctgcacaacc tgtatggcct gactgaggcc attgcctctc acagggccct ggtgaaggct |
| 3001 | agggggacta ggccctttgt gatcagcagg tctacttttg ctggccatgg caggtatgct |
| 3061 | gggcactgga ctggggatgt gtggtctagc tgggagcagc tggccagctc tgtgcctgag |
| 3121 | atcctgcagt ttaatctgct gggggtgccc ctggtggggg ctgatgtgtg tggcttcctg |
| 3181 | ggcaatacct ctgaggagct gtgtgtgagg tggactcagc tgggggcttt ctaccccttc |
| 3241 | atgagaaacc acaactctct gctgagcctg ccccaggagc cctattcttt ttctgagcct |
| 3301 | gcccagcagg ctatgaggaa ggccctgact ctgaggtatg ccctgctgcc ccacctgtat |
| 3361 | accctgttcc atcaggccca tgtggctggg gagactgtgg ccagacctct gttcctggag |
| 3421 | ttccccaagg atagctctac ttggactgtg gaccaccagc tgctgtgggg ggaggctctg |
| 3481 | ctgatcaccc ctgtgctgca ggctgggaag gctgaggtga ctggctattt ccccctgggc |
| 3541 | acctggtatg atctgcagac tgtgcccatt gaggccctgg gctctctgcc ccctccccct |
| 3601 | gctgccccca gggagcctgc catccactct gagggccagt gggtgaccct gcctgcccct |
| 3661 | ctggacacta tcaatgtgca cctgagggct ggctacatca tccccctgca gggccctggc |
| 3721 | ctgactacca ctgagtctag gcagcagccc atggccctgg ctgtggctct gaccaagggg |
| 3781 | ggggaggcca ggggggagct gttctgggat gatggggagt ctctggaggt gctggagagg |
| 3841 | ggggcctaca cccaggtgat cttcctggct aggaataaca ccattgtcaa tgagctggtg |
| 3901 | agggtgacct ctgagggggc tggcctgcag ctgcagaagg tgactgtgct gggggtggct |
| 3961 | actgcccccc agcaggtgct gagcaatggg gtgcctgtga gcaacttcac ctatagccct |
| 4021 | gacaccaagg tgctggacat ctgtgtgagc ctgctgatgg gggagcagtt cctggtgagc |
| 4081 | tggtgctaat ttaaatctcg agccgggcgg agtgtgttag tctctccaga gggaggctgg |
| 4141 | ttccccaggg aagcagagcc tgtgtgcggg cagcagctgt gtgcgggcct gggggttgtt |
| 4201 | aagtgcaatt atttttaata aaaggggcat ttggaaaaaa aaaaaaaagg tagcagtcga |
| 4261 | cagatgaatt ctgcagatct gtggcttcta gctgcccggg tggcatccct gtgacccctc |
| 4321 | cccagtgcct ctcctggccc tggaagttgc cactccagtg cccaccagcc ttgtcctaat |
| 4381 | aaaattaagt tgcatcattt tgtctgacta ggtgtccttc tataatatta tgggtgggag |
| 4441 | gggggtggta tggagcaagg ggcaagttgg gaagacaacc tgtagggcct gcggggtcta |
| 4501 | ttgggaacca agctggagtg cagtggcaca atcttggctc actgcaatct ccgcctcctg |
| 4561 | ggttcaagcg attctcctgc ctcagcctcc cgagttgttg ggattccagg catgcatgac |
| 4621 | caggctcagc taatttttgt ttttttggta gagacggggt ttcaccatat tggccaggct |
| 4681 | ggtctccaac tcctaatctc aggtgatcta cccaccttgg cctcccaaat tgctgggatt |
| 4741 | acaggcgtga accactgctc ccttccctgt ccttctgatt ttaaaaaaaa gagaaatgtt |
| 4801 | ctggcacctg cacttgcact ggggacagcc tattttgcta gtttgttttg tttcgttttg |
| 4861 | ttttgatgga gagcgtatgt tgtttaaacg cggccgcgta gataagtagc atggcgggtt |
| 4921 | aatcattaac tacaaggaac ccctagtgat ggagttggcc actccctctc tgcgcgctcg |
| 4981 | ctcgctcact gaggccgggc gaccaaaggt cgcccgacgc ccgggctttg cccgggcggc |
| 5041 | ctcagtgagc gagcgagcgc gcagttaatt aaggcgccct aggccgaccc ttagactctg |
| 5101 | tactcagttc tataaacgag ccattggata cgagatccgt agattgataa gggacacgga |
| 5161 | atatccccgg acgcaataga caccggtgga cagcttggta tcctgagcac agtcgcgcgt |
| 5221 | ccgaatctag ctctacttta gaggccccgg attctgatgg tcgtagaccg cagaaccgat |
| 5281 | tggggggatg agatctacta gttatcagca cacaattgcc cattatacgc gcgtataatg |
| 5341 | gactattgtg tgctgatata gggataacag ggtaattcta gagctagcat atggatccat |
| 5401 | cgatttgatg cggtattttc tccttacgca tctgtgcggt atttcacacc gcatacgtca |
| 5461 | aagcaaccat agtacgcgcc ctgtagcggc gcattaagcg cggcgggtgt ggtggttacg |
| 5521 | cgcagcgtga ccgctacact tgccagcgcc ctagcgcccg ctcctttcgc tttcttccct |
| 5581 | tcctttctcg ccacgttcgc cggctttccc cgtcaagctc taaatcgggg gctcccttta |
| 5641 | gggttccgat ttagtgcttt acggcacctc gaccccaaaa aacttgattt gggtgatggt |
| 5701 | tcacgtagtg ggccatcgcc ctgatagacg gtttttcgcc ctttgacgtt ggagtccacg |
| 5761 | ttctttaata gtggactctt gttccaaact ggaacaacac tcaactctat ctcgggctat |
| 5821 | tcttttgatt tataagggat tttgccgatt tcggtctatt ggttaaaaaa tgagctgatt |
| 5881 | taacaaaaat ttaacgcgaa ttttaacaaa atattaacgt ttacaatttt atggtgcact |
| 5941 | ctcagtacaa tctgctctga tgccgcatag ttaagccagc cccgacaccc gccaacaccc |
| 6001 | gctgacgcgc cctgacgggc ttgtctgctc ccggcatccg cttacagaca agctgtgacc |
| 6061 | gtctccggga gctgcatgtg tcagaggttt tcaccgtcat caccgaaacg cgcgagacga |
| 6121 | aagggcctcg tgatacgcct atttttatag gttaatgtca tgataataat ggtttcttag |
| 6181 | acgtcaggtg gcacttttcg gggaaatgtg cgcggaaccc ctatttgttt atttttctaa |
| 6241 | atacattcaa atatgtatcc gctcatgaga caataaccct gataaatgct tcaataatat |
| 6301 | tgaaaaagga agagtatgag ccatattcaa cgggaaacgt cgaggccgcg attaaattcc |
| 6361 | aacatggatg ctgatttata tgggtataaa tgggctcgcg ataatgtcgg gcaatcaggt |
| 6421 | gcgacaatct atcgcttgta tgggaagccc gatgcgccag agttgtttct gaaacatggc |
| 6481 | aaaggtagcg ttgccaatga tgttacagat gagatggtca gactaaactg gctgacggaa |
| 6541 | tttatgcctc ttccgaccat caagcatttt atccgtactc ctgatgatgc atggttactc |
| 6601 | accactgcga tccccggaaa aacagcattc caggtattag aagaatatcc tgattcaggt |
| 6661 | gaaaatattg ttgatgcgct ggcagtgttc ctgcgccggt tgcattcgat tcctgtttgt |
| 6721 | aattgtcctt ttaacagcga tcgcgtattt cgtctcgctc aggcgcaatc acgaatgaat |
| 6781 | aacggtttgg ttgatgcgag tgattttgat gacgagcgta atggctggcc tgttgaacaa |
| 6841 | gtctggaaag aaatgcataa acttttgcca ttctcaccgg attcagtcgt cactcatggt |
| 6901 | gatttctcac ttgataacct tatttttgac gaggggaaat taataggttg tattgatgtt |
| 6961 | ggacgagtcg gaatcgcaga ccgataccag gatcttgcca tcctatggaa ctgcctcggt |
| 7021 | gagttttctc cttcattaca gaaacggctt tttcaaaaat atggtattga taatcctgat |
| 7081 | atgaataaat tgcagtttca tttgatgctc gatgagtttt tctaagcgta taatggtcta |
| 7141 | gagctagcat atggatccat cgattccatt atacgcctgt cagaccaagt ttactcatat |
| 7201 | atactttaga ttgatttaaa acttcatttt taatttaaaa ggatctaggt gaagatcctt |
| 7261 | tttgataatc tcatgaccaa aatcccttaa cgtgagtttt cgttccactg agcgtcagac |
| 7321 | cccgtagaaa agatcaaagg atcttcttga gatccttttt ttctgcgcgt aatctgctgc |
| 7381 | ttgcaaacaa aaaaaccacc gctaccagcg gtggtttgtt tgccggatca agagctacca |
| 7441 | actctttttc cgaaggtaac tggcttcagc agagcgcaga taccaaatac tgttcttcta |
| 7501 | gtgtagccgt agttaggcca ccacttcaag aactctgtag caccgcctac atacctcgct |
| 7561 | ctgctaatcc tgttaccagt ggctgctgcc agtggcgata agtcgtgtct taccgggttg |
| 7621 | gactcaagac gatagttacc ggataaggcg cagcggtcgg gctgaacggg gggttcgtgc |
| 7681 | acacagccca gcttggagcg aacgacctac accgaactga gatacctaca gcgtgagcta |
| 7741 | tgagaaagcg ccacgcttcc cgaagggaga aaggcggaca ggtatccggt aagcggcagg |
| 7801 | gtcggaacag gagagcgcac gagggagctt ccagggggaa acgcctggta tctttatagt |
| 7861 | cctgtcgggt ttcgccacct ctgacttgag cgtcgatttt tgtgatgctc gtcagggggg |
| 7921 | cggagcctat ggaaaaacgc cagcaacgcg gcctttttac ggttcctggc cttttgctgg |
| 7981 | ccttttgctc acatgt |

| pP089_seq3-IL2wt_11-56 deletion (SEQ ID NO: 494) | |
|---|---|
| (SEQ ID NO: 494) | |
| 1 | tctagagcta gcatatggat ccatcgattt agggataaca gggtaattat cagcacacaa |
| 61 | ttgcccatta tacgcgcgta taatggacta ttgtgtgctg atatctgtac acttaagggc |
| 121 | tagatcttag cttacgtcac tagagggtcc acgtttagtt tttaagatcc attgatctcc |
| 181 | taaacgctgc aagattcgca acctggtata cttagcctag gcgctaggtc ctagtgcagc |
| 241 | gggacttttt ttctaaagtc gttgagagga ggagtcgtca gaccagatag ctttgatgtc |
| 301 | ctgatcggaa ggatcgttgg cccccctgca ggcagctgtt aattaactgc gcgctcgctc |
| 361 | gctcactgag gccgcccggg caaagcccgg gcgtcgggcg acctttggtc gcccggcctc |
| 421 | agtgagcgag cgagcgcgca gagagggagt ggccaactcc atcactaggg gttccttgta |
| 481 | gttaatgatt aacccgccat gctacttatc tacggcgcgc cacgcgttct aggagttaat |
| 541 | ttttaaaaag cagtcaaaag tccaagtgcc cttgcgagca tttactctct ctgtttgctc |
| 601 | tggttaataa tctcaggagc acaaacattc cttactagtt ctaggagtta atttttaaaa |
| 661 | agcagtcaaa agtccaagtg cccttgcgag catttactct ctctgtttgc tctggttaat |
| 721 | aatctcagga gcacaaacat tccttactag ttctagagcg gccgccagtg tgctggaatt |
| 781 | cggctttttt agggctggaa gctacctttg acatcatttc ctctgcgaat gcatgtataa |
| 841 | tttctacaga acctattaga aaggatcacc cagcctctgc ttttgtacaa ctttccctta |
| 901 | aaaaactgcc aatcccactg ctgtttggcc caatagtgag aactttttcc tgctgcctct |
| 961 | tggtgctttt gcctatggcc cctattctgc ctgctgaaga cactcttgcc agcatggact |
| 1021 | taaacccctc cagctctgac aatcctcttt ctcttttgtt ttacatgaag ggtctggcag |
| 1081 | ccaaagcaat cactcaaagt tcaaacctta tcattttttg ctttgttcct cttggccttg |
| 1141 | gttttgtaca tcagctttga aaataccatc ccagggttaa tgctggggtt aatttataac |
| 1201 | tgagagtgct ctagttctgc aatacaggac atgctataaa aatggaaaga tgttgctttc |
| 1261 | tgagagatca ggtggattct tgggcatttg cttaagcttg gtaccgccac catgggggtg |
| 1321 | aggcaccctc cctgttctca ctacagaatg cagctgctgt cctgcattgc actgtcactg |
| 1381 | gcactggtta ccaactcaca gcagggagct tctagacctg gacctagaga tgctcaggct |
| 1441 | catcctggaa gacctagagc tgtgcctaca cagtgtgatg tgcctcctaa ttctagattt |
| 1501 | gattgtgctc ctgataaggc tataacacag gagcagtgtg aggctagagg atgttgttat |
| 1561 | attcctgcta agcagggact gcagggagct cagatgggac agccttggtg tttttttcct |
| 1621 | ccttcttatc cttcttataa gctggagaat ctgtcttctt ctgagatggg atatacagct |
| 1681 | acactgacaa gaacaacacc tacatttttt cctaaggata ttctgacact gagactggat |
| 1741 | gtgatgatgg agacagagaa tagactgcat tttacaatta aggatcctgc taatagaaga |
| 1801 | tatgaggtgc ctctagagac acctagagtg cattctagag ctccttctcc tctgtattct |
| 1861 | gtggagtttt ctgaggagcc ttttggagtg attgtgcata gacagctgga tggaagagtg |
| 1921 | ctgctgaata caacagtggc tcctctgttt tttgctgatc agtttctgca gctgtctaca |
| 1981 | tctctgcctt ctcagtatat tacaggactg gctgagcatc tgtctcctct gatgctgtct |
| 2041 | acatcttgga caagaattac actgtggaat agagatctgg ctcctacacc tggagctaat |
| 2101 | ctgtatggat ctcatccttt ttatctggct ctggaggatg gaggatctgc tcatggagtg |
| 2161 | tttctgctga attctaatgc tatggatgta gtgctgcagc cttctcctgc tctgtcttgg |
| 2221 | agatctacag gaggaattct ggatgtgtat atttttctag gacctgagcc taagtctgtg |
| 2281 | gtgcagcagt atctggatgt agtgggatat ccttttatgc ctccttattg gggactggga |
| 2341 | tttcatctgt gtagatgggg atattcttct acagctatta caagacaggt ggttgagaat |
| 2401 | atgacaagag ctcattttcc tctggatgtg cagtggaatg atctagatta tatggattct |
| 2461 | agaagagatt ttacatttaa taaggatgga tttagagatt ttcctgctat ggtgcaggag |
| 2521 | ctgcatcagg gaggaagaag atatatgatg attgtggatc ctgctatttc ttcttctgga |
| 2581 | cctgctggat cttatagacc ttatgatgag ggactgagaa gaggagtgtt tattacaaat |
| 2641 | gagacaggac agcctctgat tggaaaggtg tggcctggat ctacagcttt tcctgatttt |
| 2701 | acaaatccta cagctctggc ttggtgggag gatatggtgg ctgagtttca tgatcaggtg |
| 2761 | ccttttgatg gaatgtggat tgatatgaat gagccttcta attttataag aggatctgag |
| 2821 | gatggatgtc ctaataatga gctagagaat cctccttatg tgcctggagt agtgggagga |
| 2881 | acactgcagg ctgctacaat ttgtgcttct tctcatcagt ttctgtctac acattataat |
| 2941 | ctgcataatc tgtatggact gacagaggct attgcttctc atagagctct ggtgaaggct |
| 3001 | agaggaacaa gaccttttgt gatttctaga tctacatttg ctggacatgg aagatatgct |
| 3061 | ggacattgga caggagatgt atggtcttct tgggagcagc tagcttcttc tgtgcctgag |
| 3121 | attctgcagt ttaatctgct aggagtgcct ctggtgggag ctgatgtgtg tggatttctg |
| 3181 | ggaaatacat ctgaggagct gtgtgtgaga tggacacagc tgggagcttt ttatcctttt |
| 3241 | atgagaaatc ataattctct gctgtctctg cctcaggagc cttattcttt ttctgagcct |
| 3301 | gctcagcagg ctatgagaaa ggctctgaca ctgagatatg ctctgctgcc tcatctgtat |
| 3361 | acactgtttc atcaggctca tgtggctgga gagacagtag ctagacctct gtttctggag |
| 3421 | tttcctaagg attcttctac atggacagtt gatcatcagc tgctgtgggg agaggctctg |
| 3481 | ctgattacac ctgtgctgca ggctggaaag gctgaggtga caggatattt tcctctggga |
| 3541 | acatggtatg atctgcagac agtgcctata gaggctctgg gatctctgcc tcctcctcct |
| 3601 | gctgctccta gagagcctgc tattcattct gagggacagt gggtgacact gcctgctcct |
| 3661 | ctggatacaa ttaatgtgca tctgagagct ggatatatta ttcctctgca gggacctgga |
| 3721 | ctgacaacaa cagagtctag acagcagcct atggctctgg ctgtggctct gacaaaggga |
| 3781 | ggagaggcta gaggagagct gttttgggat gatggagagt ctctggaggt gctggagaga |
| 3841 | ggagcttata cacaggtgat ttttctggct agaaataata caattgtgaa tgagctggtg |
| 3901 | agagtgacat ctgagggagc tggactgcag ctgcagaagg tgacagtgct gggagtggct |
| 3961 | acagctcctc agcaggtgct gtctaatgga gtgcctgtgt ctaattttac atattctcct |
| 4021 | gatacaaagg tgctggatat ttgtgtgtct ctgctgatgg gagagcagtt tctggtgtct |
| 4081 | tggtgttaat gatttaaatc tcgagccggg cggagtgtgt tagtctctcc agagggaggc |
| 4141 | tggttcccca gggaagcaga gcctgtgtgc gggcagcagc tgtgtgcggg cctgggggtt |
| 4201 | gttaagtgca attattttta ataaaagggg catttggaaa aaaaaaaaaa aggtagcagt |
| 4261 | cgacagatga attctgcaga tctgtggctt ctagctgccc gggtggcatc cctgtgaccc |
| 4321 | ctccccagtg cctctcctgg ccctggaagt tgccactcca gtgcccacca gccttgtcct |
| 4381 | aataaaatta agttgcatca ttttgtctga ctaggtgtcc ttctataata ttatggggtg |
| 4441 | gaggggggtg gtatggagca aggggcaagt tgggaagaca acctgtaggg cctgcggggt |
| 4501 | ctattgggaa ccaagctgga gtgcagtggc acaatcttgg ctcactgcaa tctccgcctc |
| 4561 | ctgggttcaa gcgattctcc tgcctcagcc tcccgagttg ttgggattcc aggcatgcat |
| 4621 | gaccaggctc agctaatttt tgtttttttg gtagagacgg ggtttcacca tattggccag |
| 4681 | gctggtctcc aactcctaat ctcaggtgat ctacccacct tggcctccca aattgctggg |
| 4741 | attacaggcg tgaaccactg ctcccttccc tgtccttctg attttaaaaa aaagagaaat |
| 4801 | gttctggcac ctgcacttgc actggggaca gcctattttg ctagtttgtt ttgtttcgtt |
| 4861 | ttgttttgat ggagagcgta tgttgtttaa acgcggccgc gtagataagt agcatggcgg |
| 4921 | gttaatcatt aactacaagg aacccctagt gatggagttg gccactccct ctctgcgcgc |
| 4981 | tcgctcgctc actgaggccg ggcgaccaaa ggtcgcccga cgcccgggct ttgcccgggc |
| 5041 | ggcctcagtg agcgagcgag cgcgcagtta attaaggcgc cctaggccga cccttagact |
| 5101 | ctgtactcag ttctataaac gagccattgg atacgagatc cgtagattga taagggacac |
| 5161 | ggaatatccc cggacgcaat agacaccggt ggacagcttg gtatcctgag cacagtcgcg |
| 5221 | cgtccgaatc tagctctact ttagaggccc cggattctga tggtcgtaga ccgcagaacc |
| 5281 | gattgggggg atgagatcta ctagttatca gcacacaatt gcccattata cgcgcgtata |
| 5341 | atggactatt gtgtgctgat atagggataa cagggtaatt ctagagctag catatggatc |
| 5401 | catcgatttg atgcggtatt ttctccttac gcatctgtgc ggtatttcac accgcatacg |
| 5461 | tcaaagcaac catagtacgc gccctgtagc ggcgcattaa gcgcggcggg tgtggtggtt |
| 5521 | acgcgcagcg tgaccgctac acttgccagc gccctagcgc ccgctccttt cgctttcttc |
| 5581 | ccttcctttc tcgccacgtt cgccggcttt ccccgtcaag ctctaaatcg ggggctccct |
| 5641 | ttagggttcc gatttagtgc tttacggcac ctcgacccca aaaaacttga tttgggtgat |
| 5701 | ggttcacgta gtgggccatc gccctgatag acggtttttc gccctttgac gttggagtcc |
| 5761 | acgttcttta atagtggact cttgttccaa actggaacaa cactcaactc tatctcgggc |
| 5821 | tattcttttg atttataagg gattttgccg atttcggtct attggttaaa aaatgagctg |
| 5881 | atttaacaaa aatttaacgc gaattttaac aaaatattaa cgtttacaat tttatggtgc |
| 5941 | actctcagta caatctgctc tgatgccgca tagttaagcc agccccgaca cccgccaaca |
| 6001 | cccgctgacg cgccctgacg ggcttgtctg ctcccggcat ccgcttacag acaagctgtg |
| 6061 | accgtctccg ggagctgcat gtgtcagagg ttttcaccgt catcaccgaa acgcgcgaga |
| 6121 | cgaaagggcc tcgtgatacg cctattttta taggttaatg tcatgataat aatggtttct |
| 6181 | tagacgtcag gtggcacttt tcggggaaat gtgcgcggaa cccctatttg tttatttttc |
| 6241 | taaatacatt caaatatgta tccgctcatg agacaataac cctgataaat gcttcaataa |
| 6301 | tattgaaaaa ggaagagtat gagccatatt caacgggaaa cgtcgaggcc gcgattaaat |
| 6361 | tccaacatgg atgctgattt atatgggtat aaatgggctc gcgataatgt cgggcaatca |
| 6421 | ggtgcgacaa tctatcgctt gtatgggaag cccgatgcgc cagagttgtt tctgaaacat |
| 6481 | ggcaaaggta gcgttgccaa tgatgttaca gatgagatgg tcagactaaa ctggctgacg |
| 6541 | gaatttatgc ctcttccgac catcaagcat tttatccgta ctcctgatga tgcatggtta |
| 6601 | ctcaccactg cgatccccgg aaaaacagca ttccaggtat tagaagaata tcctgattca |
| 6661 | ggtgaaaata ttgttgatgc gctggcagtg ttcctgcgcc ggttgcattc gattcctgtt |
| 6721 | tgtaattgtc cttttaacag cgatcgcgta tttcgtctcg ctcaggcgca atcacgaatg |
| 6781 | aataacggtt tggttgatgc gagtgatttt gatgacgagc gtaatggctg gcctgttgaa |
| 6841 | caagtctgga aagaaatgca taaacttttg ccattctcac cggattcagt cgtcactcat |
| 6901 | ggtgatttct cacttgataa ccttattttt gacgagggga aattaatagg ttgtattgat |
| 6961 | gttggacgag tcggaatcgc agaccgatac caggatcttg ccatcctatg gaactgcctc |
| 7021 | ggtgagtttt ctccttcatt acagaaacgg ctttttcaaa aatatggtat tgataatcct |
| 7081 | gatatgaata aattgcagtt tcatttgatg ctcgatgagt ttttctaagc gtataatggt |
| 7141 | ctagagctag catatggatc catcgattcc attatacgcc tgtcagacca agtttactca |
| 7201 | tatatacttt agattgattt aaaacttcat ttttaattta aaaggatcta ggtgaagatc |
| 7261 | ctttttgata atctcatgac caaaatccct taacgtgagt tttcgttcca ctgagcgtca |
| 7321 | gaccccgtag aaaagatcaa aggatcttct tgagatcctt tttttctgcg cgtaatctgc |
| 7381 | tgcttgcaaa caaaaaaacc accgctacca gcggtggttt gtttgccgga tcaagagcta |
| 7441 | ccaactcttt ttccgaaggt aactggcttc agcagagcgc agataccaaa tactgttctt |
| 7501 | ctagtgtagc cgtagttagg ccaccacttc aagaactctg tagcaccgcc tacatacctc |
| 7561 | gctctgctaa tcctgttacc agtggctgct gccagtggcg ataagtcgtg tcttaccggg |
| 7621 | ttggactcaa gacgatagtt accggataag gcgcagcggt cgggctgaac ggggggttcg |
| 7681 | tgcacacagc ccagcttgga gcgaacgacc tacaccgaac tgagatacct acagcgtgag |
| 7741 | ctatgagaaa gcgccacgct tcccgaaggg agaaaggcgg acaggtatcc ggtaagcggc |
| 7801 | agggtcggaa caggagagcg cacgagggag cttccagggg gaaacgcctg gtatctttat |
| 7861 | agtcctgtcg ggtttcgcca cctctgactt gagcgtcgat ttttgtgatg ctcgtcaggg |
| 7921 | gggcggagcc tatggaaaaa cgccagcaac gcggcctttt tacggttcct ggccttttgc |
| 7981 | tggccttttg ctcacatgt |

| pP090_seq3-201Ig_11-56 deletion (SEQ ID NO: 495) | |
|---|---|
| (SEQ ID NO: 495) | |
| 1 | tctagagcta gcatatggat ccatcgattt agggataaca gggtaattat cagcacacaa |
| 61 | ttgcccatta tacgcgcgta taatggacta ttgtgtgctg atatctgtac acttaagggc |
| 121 | tagatcttag cttacgtcac tagagggtcc acgtttagtt tttaagatcc attgatctcc |
| 181 | taaacgctgc aagattcgca acctggtata cttagcctag gcgctaggtc ctagtgcagc |
| 241 | gggacttttt ttctaaagtc gttgagagga ggagtcgtca gaccagatag ctttgatgtc |
| 301 | ctgatcggaa ggatcgttgg cccccctgca ggcagctgtt aattaactgc gcgctcgctc |
| 361 | gctcactgag gccgcccggg caaagcccgg gcgtcgggcg acctttggtc gcccggcctc |
| 421 | agtgagcgag cgagcgcgca gagagggagt ggccaactcc atcactaggg gttccttgta |
| 481 | gttaatgatt aacccgccat gctacttatc tacggcgcgc cacgcgttct aggagttaat |
| 541 | ttttaaaaag cagtcaaaag tccaagtgcc cttgcgagca tttactctct ctgtttgctc |
| 601 | tggttaataa tctcaggagc acaaacattc cttactagtt ctaggagtta atttttaaaa |
| 661 | agcagtcaaa agtccaagtg cccttgcgag catttactct ctctgtttgc tctggttaat |
| 721 | aatctcagga gcacaaacat tccttactag ttctagagcg gccgccagtg tgctggaatt |
| 781 | cggctttttt agggctggaa gctacctttg acatcatttc ctctgcgaat gcatgtataa |
| 841 | tttctacaga acctattaga aaggatcacc cagcctctgc ttttgtacaa ctttccctta |
| 901 | aaaaactgcc aatcccactg ctgtttggcc caatagtgag aactttttcc tgctgcctct |
| 961 | tggtgctttt gcctatggcc cctattctgc ctgctgaaga cactcttgcc agcatggact |
| 1021 | taaacccctc cagctctgac aatcctcttt ctcttttgtt ttacatgaag ggtctggcag |
| 1081 | ccaaagcaat cactcaaagt tcaaacctta tcattttttg ctttgttcct cttggccttg |
| 1141 | gttttgtaca tcagctttga aaataccatc ccagggttaa tgctggggtt aatttataac |
| 1201 | tgagagtgct ctagttctgc aatacaggac atgctataaa aatggaaaga tgttgctttc |
| 1261 | tgagagatca ggtggattct tgggcatttg cttaagcttg gtaccgccac catgggggtg |
| 1321 | aggcaccctc cctgttctca tgagtttggg ctgagctggg tctttctggt ggccctgctg |
| 1381 | aagggagtcc agtgtgagca gcagggagct tctagacctg gacctagaga tgctcaggct |
| 1441 | catcctggaa gacctagagc tgtgcctaca cagtgtgatg tgcctcctaa ttctagattt |
| 1501 | gattgtgctc ctgataaggc tataacacag gagcagtgtg aggctagagg atgttgttat |
| 1561 | attcctgcta agcagggact gcagggagct cagatgggac agccttggtg tttttttcct |
| 1621 | ccttcttatc cttcttataa gctggagaat ctgtcttctt ctgagatggg atatacagct |
| 1681 | acactgacaa gaacaacacc tacatttttt cctaaggata ttctgacact gagactggat |
| 1741 | gtgatgatgg agacagagaa tagactgcat tttacaatta aggatcctgc taatagaaga |
| 1801 | tatgaggtgc ctctagagac acctagagtg cattctagag ctccttctcc tctgtattct |
| 1861 | gtggagtttt ctgaggagcc ttttggagtg attgtgcata gacagctgga tggaagagtg |
| 1921 | ctgctgaata caacagtggc tcctctgttt tttgctgatc agtttctgca gctgtctaca |
| 1981 | tctctgcctt ctcagtatat tacaggactg gctgagcatc tgtctcctct gatgctgtct |
| 2041 | acatcttgga caagaattac actgtggaat agagatctgg ctcctacacc tggagctaat |
| 2101 | ctgtatggat ctcatccttt ttatctggct ctggaggatg gaggatctgc tcatggagtg |
| 2161 | tttctgctga attctaatgc tatggatgta gtgctgcagc cttctcctgc tctgtcttgg |
| 2221 | agatctacag gaggaattct ggatgtgtat atttttctag gacctgagcc taagtctgtg |
| 2281 | gtgcagcagt atctggatgt agtgggatat ccttttatgc ctccttattg gggactggga |
| 2341 | tttcatctgt gtagatgggg atattcttct acagctatta caagacaggt ggttgagaat |
| 2401 | atgacaagag ctcattttcc tctggatgtg cagtggaatg atctagatta tatggattct |
| 2461 | agaagagatt ttacatttaa taaggatgga tttagagatt ttcctgctat ggtgcaggag |
| 2521 | ctgcatcagg gaggaagaag atatatgatg attgtggatc ctgctatttc ttcttctgga |
| 2581 | cctgctggat cttatagacc ttatgatgag ggactgagaa gaggagtgtt tattacaaat |
| 2641 | gagacaggac agcctctgat tggaaaggtg tggcctggat ctacagcttt tcctgatttt |
| 2701 | acaaatccta cagctctggc ttggtgggag gatatggtgg ctgagtttca tgatcaggtg |
| 2761 | ccttttgatg gaatgtggat tgatatgaat gagccttcta attttataag aggatctgag |
| 2821 | gatggatgtc ctaataatga gctagagaat cctccttatg tgcctggagt agtgggagga |
| 2881 | acactgcagg ctgctacaat ttgtgcttct tctcatcagt ttctgtctac acattataat |
| 2941 | ctgcataatc tgtatggact gacagaggct attgcttctc atagagctct ggtgaaggct |
| 3001 | agaggaacaa gaccttttgt gatttctaga tctacatttg ctggacatgg aagatatgct |
| 3061 | ggacattgga caggagatgt atggtcttct tgggagcagc tagcttcttc tgtgcctgag |
| 3121 | attctgcagt ttaatctgct aggagtgcct ctggtgggag ctgatgtgtg tggatttctg |
| 3181 | ggaaatacat ctgaggagct gtgtgtgaga tggacacagc tgggagcttt ttatcctttt |
| 3241 | atgagaaatc ataattctct gctgtctctg cctcaggagc cttattcttt ttctgagcct |
| 3301 | gctcagcagg ctatgagaaa ggctctgaca ctgagatatg ctctgctgcc tcatctgtat |
| 3361 | acactgtttc atcaggctca tgtggctgga gagacagtag ctagacctct gtttctggag |
| 3421 | tttcctaagg attcttctac atggacagtt gatcatcagc tgctgtgggg agaggctctg |
| 3481 | ctgattacac ctgtgctgca ggctggaaag gctgaggtga caggatattt tcctctggga |
| 3541 | acatggtatg atctgcagac agtgcctata gaggctctgg gatctctgcc tcctcctcct |
| 3601 | gctgctccta gagagcctgc tattcattct gagggacagt gggtgacact gcctgctcct |
| 3661 | ctggatacaa ttaatgtgca tctgagagct ggatatatta ttcctctgca gggacctgga |
| 3721 | ctgacaacaa cagagtctag acagcagcct atggctctgg ctgtggctct gacaaaggga |
| 3781 | ggagaggcta gaggagagct gttttgggat gatggagagt ctctggaggt gctggagaga |
| 3841 | ggagcttata cacaggtgat ttttctggct agaaataata caattgtgaa tgagctggtg |
| 3901 | agagtgacat ctgagggagc tggactgcag ctgcagaagg tgacagtgct gggagtggct |
| 3961 | acagctcctc agcaggtgct gtctaatgga gtgcctgtgt ctaattttac atattctcct |
| 4021 | gatacaaagg tgctggatat ttgtgtgtct ctgctgatgg gagagcagtt tctggtgtct |
| 4081 | tggtgttaat gatttaaatc tcgagccggg cggagtgtgt tagtctctcc agagggaggc |
| 4141 | tggttcccca gggaagcaga gcctgtgtgc gggcagcagc tgtgtgcggg cctgggggtt |
| 4201 | gttaagtgca attattttta ataaaagggg catttggaaa aaaaaaaaaa aggtagcagt |
| 4261 | cgacagatga attctgcaga tctgtggctt ctagctgccc gggtggcatc cctgtgaccc |
| 4321 | ctccccagtg cctctcctgg ccctggaagt tgccactcca gtgcccacca gccttgtcct |
| 4381 | aataaaatta agttgcatca ttttgtctga ctaggtgtcc ttctataata ttatggggtg |
| 4441 | gaggggggtg gtatggagca aggggcaagt tgggaagaca acctgtaggg cctgcggggt |
| 4501 | ctattgggaa ccaagctgga gtgcagtggc acaatcttgg ctcactgcaa tctccgcctc |
| 4561 | ctgggttcaa gcgattctcc tgcctcagcc tcccgagttg ttgggattcc aggcatgcat |
| 4621 | gaccaggctc agctaatttt tgtttttttg gtagagacgg ggtttcacca tattggccag |
| 4681 | gctggtctcc aactcctaat ctcaggtgat ctacccacct tggcctccca aattgctggg |
| 4741 | attacaggcg tgaaccactg ctcccttccc tgtccttctg attttaaaaa aaagagaaat |
| 4801 | gttctggcac ctgcacttgc actggggaca gcctattttg ctagtttgtt ttgtttcgtt |
| 4861 | ttgttttgat ggagagcgta tgttgtttaa acgcggccgc gtagataagt agcatggcgg |
| 4921 | gttaatcatt aactacaagg aacccctagt gatggagttg gccactccct ctctgcgcgc |
| 4981 | tcgctcgctc actgaggccg ggcgaccaaa ggtcgcccga cgcccgggct ttgcccgggc |
| 5041 | ggcctcagtg agcgagcgag cgcgcagtta attaaggcgc cctaggccga cccttagact |
| 5101 | ctgtactcag ttctataaac gagccattgg atacgagatc cgtagattga taagggacac |
| 5161 | ggaatatccc cggacgcaat agacaccggt ggacagcttg gtatcctgag cacagtcgcg |
| 5221 | cgtccgaatc tagctctact ttagaggccc cggattctga tggtcgtaga ccgcagaacc |
| 5281 | gattgggggg atgagatcta ctagttatca gcacacaatt gcccattata cgcgcgtata |
| 5341 | atggactatt gtgtgctgat atagggataa cagggtaatt ctagagctag catatggatc |
| 5401 | catcgatttg atgcggtatt ttctccttac gcatctgtgc ggtatttcac accgcatacg |
| 5461 | tcaaagcaac catagtacgc gccctgtagc ggcgcattaa gcgcggcggg tgtggtggtt |
| 5521 | acgcgcagcg tgaccgctac acttgccagc gccctagcgc ccgctccttt cgctttcttc |
| 5581 | ccttcctttc tcgccacgtt cgccggcttt ccccgtcaag ctctaaatcg ggggctccct |
| 5641 | ttagggttcc gatttagtgc tttacggcac ctcgacccca aaaaacttga tttgggtgat |
| 5701 | ggttcacgta gtgggccatc gccctgatag acggtttttc gccctttgac gttggagtcc |
| 5761 | acgttcttta atagtggact cttgttccaa actggaacaa cactcaactc tatctcgggc |
| 5821 | tattcttttg atttataagg gattttgccg atttcggtct attggttaaa aaatgagctg |
| 5881 | atttaacaaa aatttaacgc gaattttaac aaaatattaa cgtttacaat tttatggtgc |
| 5941 | actctcagta caatctgctc tgatgccgca tagttaagcc agccccgaca cccgccaaca |
| 6001 | cccgctgacg cgccctgacg ggcttgtctg ctcccggcat ccgcttacag acaagctgtg |
| 6061 | accgtctccg ggagctgcat gtgtcagagg ttttcaccgt catcaccgaa acgcgcgaga |
| 6121 | cgaaagggcc tcgtgatacg cctattttta taggttaatg tcatgataat aatggtttct |
| 6181 | tagacgtcag gtggcacttt tcggggaaat gtgcgcggaa cccctatttg tttatttttc |
| 6241 | taaatacatt caaatatgta tccgctcatg agacaataac cctgataaat gcttcaataa |
| 6301 | tattgaaaaa ggaagagtat gagccatatt caacgggaaa cgtcgaggcc gcgattaaat |
| 6361 | tccaacatgg atgctgattt atatgggtat aaatgggctc gcgataatgt cgggcaatca |
| 6421 | ggtgcgacaa tctatcgctt gtatgggaag cccgatgcgc cagagttgtt tctgaaacat |
| 6481 | ggcaaaggta gcgttgccaa tgatgttaca gatgagatgg tcagactaaa ctggctgacg |
| 6541 | gaatttatgc ctcttccgac catcaagcat tttatccgta ctcctgatga tgcatggtta |
| 6601 | ctcaccactg cgatccccgg aaaaacagca ttccaggtat tagaagaata tcctgattca |
| 6661 | ggtgaaaata ttgttgatgc gctggcagtg ttcctgcgcc ggttgcattc gattcctgtt |
| 6721 | tgtaattgtc cttttaacag cgatcgcgta tttcgtctcg ctcaggcgca atcacgaatg |
| 6781 | aataacggtt tggttgatgc gagtgatttt gatgacgagc gtaatggctg gcctgttgaa |
| 6841 | caagtctgga aagaaatgca taaacttttg ccattctcac cggattcagt cgtcactcat |
| 6901 | ggtgatttct cacttgataa ccttattttt gacgagggga aattaatagg ttgtattgat |
| 6961 | gttggacgag tcggaatcgc agaccgatac caggatcttg ccatcctatg gaactgcctc |
| 7021 | ggtgagtttt ctccttcatt acagaaacgg ctttttcaaa aatatggtat tgataatcct |
| 7081 | gatatgaata aattgcagtt tcatttgatg ctcgatgagt ttttctaagc gtataatggt |
| 7141 | ctagagctag catatggatc catcgattcc attatacgcc tgtcagacca agtttactca |
| 7201 | tatatacttt agattgattt aaaacttcat ttttaattta aaaggatcta ggtgaagatc |
| 7261 | ctttttgata atctcatgac caaaatccct taacgtgagt tttcgttcca ctgagcgtca |
| 7321 | gaccccgtag aaaagatcaa aggatcttct tgagatcctt tttttctgcg cgtaatctgc |
| 7381 | tgcttgcaaa caaaaaaacc accgctacca gcggtggttt gtttgccgga tcaagagcta |
| 7441 | ccaactcttt ttccgaaggt aactggcttc agcagagcgc agataccaaa tactgttctt |
| 7501 | ctagtgtagc cgtagttagg ccaccacttc aagaactctg tagcaccgcc tacatacctc |
| 7561 | gctctgctaa tcctgttacc agtggctgct gccagtggcg ataagtcgtg tcttaccggg |
| 7621 | ttggactcaa gacgatagtt accggataag gcgcagcggt cgggctgaac ggggggttcg |
| 7681 | tgcacacagc ccagcttgga gcgaacgacc tacaccgaac tgagatacct acagcgtgag |
| 7741 | ctatgagaaa gcgccacgct tcccgaaggg agaaaggcgg acaggtatcc ggtaagcggc |
| 7801 | agggtcggaa caggagagcg cacgagggag cttccagggg gaaacgcctg gtatctttat |
| 7861 | agtcctgtcg ggtttcgcca cctctgactt gagcgtcgat ttttgtgatg ctcgtcaggg |
| 7921 | gggcggagcc tatggaaaaa cgccagcaac gcggcctttt tacggttcct ggccttttgc |
| 7981 | tggccttttg ctcacatgt |

| pP091_seq100-IL2wt_28-56 deletion (SEQ ID NO: 496) | |
|---|---|
| (SEQ ID NO: 496) | |
| 1 | tctagagcta gcatatggat ccatcgattt agggataaca gggtaattat cagcacacaa |
| 61 | ttgcccatta tacgcgcgta taatggacta ttgtgtgctg atatctgtac acttaagggc |
| 121 | tagatcttag cttacgtcac tagagggtcc acgtttagtt tttaagatcc attgatctcc |
| 181 | taaacgctgc aagattcgca acctggtata cttagcctag gcgctaggtc ctagtgcagc |
| 241 | gggacttttt ttctaaagtc gttgagagga ggagtcgtca gaccagatag ctttgatgtc |
| 301 | ctgatcggaa ggatcgttgg cccccctgca ggcagctgtt aattaactgc gcgctcgctc |
| 361 | gctcactgag gccgcccggg caaagcccgg gcgtcgggcg acctttggtc gcccggcctc |
| 421 | agtgagcgag cgagcgcgca gagagggagt ggccaactcc atcactaggg gttccttgta |
| 481 | gttaatgatt aacccgccat gctacttatc tacggcgcgc cacgcgttct aggagttaat |
| 541 | ttttaaaaag cagtcaaaag tccaagtgcc cttgcgagca tttactctct ctgtttgctc |
| 601 | tggttaataa tctcaggagc acaaacattc cttactagtt ctaggagtta atttttaaaa |
| 661 | agcagtcaaa agtccaagtg cccttgcgag catttactct ctctgtttgc tctggttaat |
| 721 | aatctcagga gcacaaacat tccttactag ttctagagcg gccgccagtg tgctggaatt |
| 781 | cggctttttt agggctggaa gctacctttg acatcatttc ctctgcgaat gcatgtataa |
| 841 | tttctacaga acctattaga aaggatcacc cagcctctgc ttttgtacaa ctttccctta |
| 901 | aaaaactgcc aatcccactg ctgtttggcc caatagtgag aactttttcc tgctgcctct |
| 961 | tggtgctttt gcctatggcc cctattctgc ctgctgaaga cactcttgcc agcatggact |
| 1021 | taaacccctc cagctctgac aatcctcttt ctcttttgtt ttacatgaag ggtctggcag |
| 1081 | ccaaagcaat cactcaaagt tcaaacctta tcattttttg ctttgttcct cttggccttg |
| 1141 | gttttgtaca tcagctttga aaataccatc ccagggttaa tgctggggtt aatttataac |
| 1201 | tgagagtgct ctagttctgc aatacaggac atgctataaa aatggaaaga tgttgctttc |
| 1261 | tgagagatca ggtggattct tgggcatttg cttaagcttg gtaccgccac catgggggtg |
| 1321 | aggcaccctc cctgttctca caggctgctg gctgtgtgtg ctctggtgtc tctggccact |
| 1381 | gctgccctgc tgtacagaat gcagctgctg tcctgcattg cactgtcact ggcactggtt |
| 1441 | accaactcac agcagggggc cagcaggcca ggccccaggg atgcccaggc ccaccctggc |
| 1501 | agacccaggg ctgtgcccac ccagtgtgat gtgcctccca acagcagatt tgactgtgcc |
| 1561 | cctgataagg ccattactca ggaacagtgt gaggccaggg gctgctgcta tatccctgcc |
| 1621 | aagcagggcc tgcagggggc ccagatgggg cagccctggt gcttcttccc ccccagctac |
| 1681 | ccctcttata agctggagaa tctgagcagc tctgaaatgg gctacactgc cactctgacc |
| 1741 | aggactaccc ccaccttctt ccccaaggat attctgactc tgaggctgga tgtgatgatg |
| 1801 | gagactgaga acaggctgca cttcaccatc aaggaccctg ccaacaggag gtatgaggtg |
| 1861 | cccctggaaa ctcccagggt gcactctagg gcccccagcc ccctgtattc tgtggagttc |
| 1921 | tctgaggagc cttttggggt cattgtccac aggcagctgg atggcagggt gctgctgaat |
| 1981 | actactgtgg cccctctgtt ctttgctgac cagttcctgc agctgagcac cagcctgcct |
| 2041 | tctcagtaca ttactggcct ggctgagcat ctgagccccc tgatgctgag cacctcttgg |
| 2101 | accagaatca ccctgtggaa cagggacctg gctcccactc ctggggccaa cctgtatggc |
| 2161 | agccacccct tctacctggc cctggaggat gggggctctg cccatggggt gttcctgctg |
| 2221 | aacagcaatg ccatggatgt ggtgctgcag ccctctcctg ccctgtcttg gagatctact |
| 2281 | gggggcatcc tggatgtgta tatcttcctg gggcctgagc ccaagtctgt ggtgcagcag |
| 2341 | tacctggatg tggtgggcta ccccttcatg cccccctact ggggcctggg cttccacctg |
| 2401 | tgcaggtggg gctactcttc tactgctatc accaggcagg tggtggagaa catgaccagg |
| 2461 | gctcacttcc ctctggatgt gcagtggaat gacctggact acatggactc taggagagac |
| 2521 | ttcactttta ataaggatgg gttcagggac tttcctgcca tggtgcagga gctgcatcag |
| 2581 | gggggcagga gatatatgat gattgtggac cctgctattt ctagctctgg ccctgctggc |
| 2641 | agctataggc cctatgatga ggggctgagg aggggggtgt tcatcactaa tgagactggc |
| 2701 | cagcccctga ttggcaaggt gtggcctggc tctactgcct tccctgattt caccaacccc |
| 2761 | actgccctgg cctggtggga ggatatggtg gctgagtttc atgaccaggt gccctttgat |
| 2821 | ggcatgtgga ttgacatgaa tgagcccagc aactttatca ggggctctga agatggctgc |
| 2881 | cccaacaatg agctggagaa ccccccctat gtgcctgggg tggtgggggg caccctgcag |
| 2941 | gctgccacca tctgtgccag cagccaccag ttcctgagca cccactataa cctgcacaac |
| 3001 | ctgtatggcc tgactgaggc cattgcctct cacagggccc tggtgaaggc tagggggact |
| 3061 | aggccctttg tgatcagcag gtctactttt gctggccatg gcaggtatgc tgggcactgg |
| 3121 | actggggatg tgtggtctag ctgggagcag ctggccagct ctgtgcctga gatcctgcag |
| 3181 | tttaatctgc tgggggtgcc cctggtgggg gctgatgtgt gtggcttcct gggcaatacc |
| 3241 | tctgaggagc tgtgtgtgag gtggactcag ctgggggctt tctacccctt catgagaaac |
| 3301 | cacaactctc tgctgagcct gccccaggag ccctattctt tttctgagcc tgcccagcag |
| 3361 | gctatgagga aggccctgac tctgaggtat gccctgctgc cccacctgta taccctgttc |
| 3421 | catcaggccc atgtggctgg ggagactgtg gccagacctc tgttcctgga gttccccaag |
| 3481 | gatagctcta cttggactgt ggaccaccag ctgctgtggg gggaggctct gctgatcacc |
| 3541 | cctgtgctgc aggctgggaa ggctgaggtg actggctatt tccccctggg cacctggtat |
| 3601 | gatctgcaga ctgtgcccat tgaggccctg ggctctctgc cccctccccc tgctgccccc |
| 3661 | agggagcctg ccatccactc tgagggccag tgggtgaccc tgcctgcccc tctggacact |
| 3721 | atcaatgtgc acctgagggc tggctacatc atccccctgc agggccctgg cctgactacc |
| 3781 | actgagtcta ggcagcagcc catggccctg gctgtggctc tgaccaaggg gggggaggcc |
| 3841 | aggggggagc tgttctggga tgatggggag tctctggagg tgctggagag gggggcctac |
| 3901 | acccaggtga tcttcctggc taggaataac accattgtca atgagctggt gagggtgacc |
| 3961 | tctgaggggg ctggcctgca gctgcagaag gtgactgtgc tgggggtggc tactgccccc |
| 4021 | cagcaggtgc tgagcaatgg ggtgcctgtg agcaacttca cctatagccc tgacaccaag |
| 4081 | gtgctggaca tctgtgtgag cctgctgatg ggggagcagt tcctggtgag ctggtgctaa |
| 4141 | tttaaatctc gagccgggcg gagtgtgtta gtctctccag agggaggctg gttccccagg |
| 4201 | gaagcagagc ctgtgtgcgg gcagcagctg tgtgcgggcc tgggggttgt taagtgcaat |
| 4261 | tatttttaat aaaaggggca tttggaaaaa aaaaaaaaag gtagcagtcg acagatgaat |
| 4321 | tctgcagatc tgtggcttct agctgcccgg gtggcatccc tgtgacccct ccccagtgcc |
| 4381 | tctcctggcc ctggaagttg ccactccagt gcccaccagc cttgtcctaa taaaattaag |
| 4441 | ttgcatcatt ttgtctgact aggtgtcctt ctataatatt atggggtgga ggggggtggt |
| 4501 | atggagcaag gggcaagttg ggaagacaac ctgtagggcc tgcggggtct attgggaacc |
| 4561 | aagctggagt gcagtggcac aatcttggct cactgcaatc tccgcctcct gggttcaagc |
| 4621 | gattctcctg cctcagcctc ccgagttgtt gggattccag gcatgcatga ccaggctcag |
| 4681 | ctaatttttg tttttttggt agagacgggg tttcaccata ttggccaggc tggtctccaa |
| 4741 | ctcctaatct caggtgatct acccaccttg gcctcccaaa ttgctgggat tacaggcgtg |
| 4801 | aaccactgct cccttccctg tccttctgat tttaaaaaaa agagaaatgt tctggcacct |
| 4861 | gcacttgcac tggggacagc ctattttgct agtttgtttt gtttcgtttt gttttgatgg |
| 4921 | agagcgtatg ttgtttaaac gcggccgcgt agataagtag catggcgggt taatcattaa |
| 4981 | ctacaaggaa cccctagtga tggagttggc cactccctct ctgcgcgctc gctcgctcac |
| 5041 | tgaggccggg cgaccaaagg tcgcccgacg cccgggcttt gcccgggcgg cctcagtgag |
| 5101 | cgagcgagcg cgcagttaat taaggcgccc taggccgacc cttagactct gtactcagtt |
| 5161 | ctataaacga gccattggat acgagatccg tagattgata agggacacgg aatatccccg |
| 5221 | gacgcaatag acaccggtgg acagcttggt atcctgagca cagtcgcgcg tccgaatcta |
| 5281 | gctctacttt agaggccccg gattctgatg gtcgtagacc gcagaaccga ttggggggat |
| 5341 | gagatctact agttatcagc acacaattgc ccattatacg cgcgtataat ggactattgt |
| 5401 | gtgctgatat agggataaca gggtaattct agagctagca tatggatcca tcgatttgat |
| 5461 | gcggtatttt ctccttacgc atctgtgcgg tatttcacac cgcatacgtc aaagcaacca |
| 5521 | tagtacgcgc cctgtagcgg cgcattaagc gcggcgggtg tggtggttac gcgcagcgtg |
| 5581 | accgctacac ttgccagcgc cctagcgccc gctcctttcg ctttcttccc ttcctttctc |
| 5641 | gccacgttcg ccggctttcc ccgtcaagct ctaaatcggg ggctcccttt agggttccga |
| 5701 | tttagtgctt tacggcacct cgaccccaaa aaacttgatt tgggtgatgg ttcacgtagt |
| 5761 | gggccatcgc cctgatagac ggtttttcgc cctttgacgt tggagtccac gttctttaat |
| 5821 | agtggactct tgttccaaac tggaacaaca ctcaactcta tctcgggcta ttcttttgat |
| 5881 | ttataaggga ttttgccgat ttcggtctat tggttaaaaa atgagctgat ttaacaaaaa |
| 5941 | tttaacgcga attttaacaa aatattaacg tttacaattt tatggtgcac tctcagtaca |
| 6001 | atctgctctg atgccgcata gttaagccag ccccgacacc cgccaacacc cgctgacgcg |
| 6061 | ccctgacggg cttgtctgct cccggcatcc gcttacagac aagctgtgac cgtctccggg |
| 6121 | agctgcatgt gtcagaggtt ttcaccgtca tcaccgaaac gcgcgagacg aaagggcctc |
| 6181 | gtgatacgcc tatttttata ggttaatgtc atgataataa tggtttctta gacgtcaggt |
| 6241 | ggcacttttc ggggaaatgt gcgcggaacc cctatttgtt tatttttcta aatacattca |
| 6301 | aatatgtatc cgctcatgag acaataaccc tgataaatgc ttcaataata ttgaaaaagg |
| 6361 | aagagtatga gccatattca acgggaaacg tcgaggccgc gattaaattc caacatggat |
| 6421 | gctgatttat atgggtataa atgggctcgc gataatgtcg ggcaatcagg tgcgacaatc |
| 6481 | tatcgcttgt atgggaagcc cgatgcgcca gagttgtttc tgaaacatgg caaaggtagc |
| 6541 | gttgccaatg atgttacaga tgagatggtc agactaaact ggctgacgga atttatgcct |
| 6601 | cttccgacca tcaagcattt tatccgtact cctgatgatg catggttact caccactgcg |
| 6661 | atccccggaa aaacagcatt ccaggtatta gaagaatatc ctgattcagg tgaaaatatt |
| 6721 | gttgatgcgc tggcagtgtt cctgcgccgg ttgcattcga ttcctgtttg taattgtcct |
| 6781 | tttaacagcg atcgcgtatt tcgtctcgct caggcgcaat cacgaatgaa taacggtttg |
| 6841 | gttgatgcga gtgattttga tgacgagcgt aatggctggc ctgttgaaca agtctggaaa |
| 6901 | gaaatgcata aacttttgcc attctcaccg gattcagtcg tcactcatgg tgatttctca |
| 6961 | cttgataacc ttatttttga cgaggggaaa ttaataggtt gtattgatgt tggacgagtc |
| 7021 | ggaatcgcag accgatacca ggatcttgcc atcctatgga actgcctcgg tgagttttct |
| 7081 | ccttcattac agaaacggct ttttcaaaaa tatggtattg ataatcctga tatgaataaa |
| 7141 | ttgcagtttc atttgatgct cgatgagttt ttctaagcgt ataatggtct agagctagca |
| 7201 | tatggatcca tcgattccat tatacgcctg tcagaccaag tttactcata tatactttag |
| 7261 | attgatttaa aacttcattt ttaatttaaa aggatctagg tgaagatcct ttttgataat |
| 7321 | ctcatgacca aaatccctta acgtgagttt tcgttccact gagcgtcaga ccccgtagaa |
| 7381 | aagatcaaag gatcttcttg agatcctttt tttctgcgcg taatctgctg cttgcaaaca |
| 7441 | aaaaaaccac cgctaccagc ggtggtttgt ttgccggatc aagagctacc aactcttttt |
| 7501 | ccgaaggtaa ctggcttcag cagagcgcag ataccaaata ctgttcttct agtgtagccg |
| 7561 | tagttaggcc accacttcaa gaactctgta gcaccgccta catacctcgc tctgctaatc |
| 7621 | ctgttaccag tggctgctgc cagtggcgat aagtcgtgtc ttaccgggtt ggactcaaga |
| 7681 | cgatagttac cggataaggc gcagcggtcg ggctgaacgg ggggttcgtg cacacagccc |
| 7741 | agcttggagc gaacgaccta caccgaactg agatacctac agcgtgagct atgagaaagc |
| 7801 | gccacgcttc ccgaagggag aaaggcggac aggtatccgg taagcggcag ggtcggaaca |
| 7861 | ggagagcgca cgagggagct tccaggggga aacgcctggt atctttatag tcctgtcggg |
| 7921 | tttcgccacc tctgacttga gcgtcgattt ttgtgatgct cgtcaggggg gcggagccta |
| 7981 | tggaaaaacg ccagcaacgc ggccttttta cggttcctgg ccttttgctg gccttttgct |
| 8041 | cacatgt |

| pP092_seq100-201Ig_28-56 deletion (SEQ ID NO: 497) | |
|---|---|
| (SEQ ID NO: 497) | |
| 1 | tctagagcta gcatatggat ccatcgattt agggataaca gggtaattat cagcacacaa |
| 61 | ttgcccatta tacgcgcgta taatggacta ttgtgtgctg atatctgtac acttaagggc |
| 121 | tagatcttag cttacgtcac tagagggtcc acgtttagtt tttaagatcc attgatctcc |
| 181 | taaacgctgc aagattcgca acctggtata cttagcctag gcgctaggtc ctagtgcagc |
| 241 | gggacttttt ttctaaagtc gttgagagga ggagtcgtca gaccagatag ctttgatgtc |
| 301 | ctgatcggaa ggatcgttgg cccccctgca ggcagctgtt aattaactgc gcgctcgctc |
| 361 | gctcactgag gccgcccggg caaagcccgg gcgtcgggcg acctttggtc gcccggcctc |
| 421 | agtgagcgag cgagcgcgca gagagggagt ggccaactcc atcactaggg gttccttgta |
| 481 | gttaatgatt aacccgccat gctacttatc tacggcgcgc cacgcgttct aggagttaat |
| 541 | ttttaaaaag cagtcaaaag tccaagtgcc cttgcgagca tttactctct ctgtttgctc |
| 601 | tggttaataa tctcaggagc acaaacattc cttactagtt ctaggagtta atttttaaaa |
| 661 | agcagtcaaa agtccaagtg cccttgcgag catttactct ctctgtttgc tctggttaat |
| 721 | aatctcagga gcacaaacat tccttactag ttctagagcg gccgccagtg tgctggaatt |
| 781 | cggctttttt agggctggaa gctacctttg acatcatttc ctctgcgaat gcatgtataa |
| 841 | tttctacaga acctattaga aaggatcacc cagcctctgc ttttgtacaa ctttccctta |
| 901 | aaaaactgcc aatcccactg ctgtttggcc caatagtgag aactttttcc tgctgcctct |
| 961 | tggtgctttt gcctatggcc cctattctgc ctgctgaaga cactcttgcc agcatggact |
| 1021 | taaacccctc cagctctgac aatcctcttt ctcttttgtt ttacatgaag ggtctggcag |
| 1081 | ccaaagcaat cactcaaagt tcaaacctta tcattttttg ctttgttcct cttggccttg |
| 1141 | gttttgtaca tcagctttga aaataccatc ccagggttaa tgctggggtt aatttataac |
| 1201 | tgagagtgct ctagttctgc aatacaggac atgctataaa aatggaaaga tgttgctttc |
| 1261 | tgagagatca ggtggattct tgggcatttg cttaagcttg gtaccgccac catgggggtg |
| 1321 | aggcaccctc cctgttctca caggctgctg gctgtgtgtg ctctggtgtc tctggccact |
| 1381 | gctgccctgc tggagtttgg gctgagctgg gtctttctgg tggccctgct gaagggagtc |
| 1441 | cagtgtgagc agcagggggc cagcaggcca ggccccaggg atgcccaggc ccaccctggc |
| 1501 | agacccaggg ctgtgcccac ccagtgtgat gtgcctccca acagcagatt tgactgtgcc |
| 1561 | cctgataagg ccattactca ggaacagtgt gaggccaggg gctgctgcta tatccctgcc |
| 1621 | aagcagggcc tgcagggggc ccagatgggg cagccctggt gcttcttccc ccccagctac |
| 1681 | ccctcttata agctggagaa tctgagcagc tctgaaatgg gctacactgc cactctgacc |
| 1741 | aggactaccc ccaccttctt ccccaaggat attctgactc tgaggctgga tgtgatgatg |
| 1801 | gagactgaga acaggctgca cttcaccatc aaggaccctg ccaacaggag gtatgaggtg |
| 1861 | cccctggaaa ctcccagggt gcactctagg gcccccagcc ccctgtattc tgtggagttc |
| 1921 | tctgaggagc cttttggggt cattgtccac aggcagctgg atggcagggt gctgctgaat |
| 1981 | actactgtgg cccctctgtt ctttgctgac cagttcctgc agctgagcac cagcctgcct |
| 2041 | tctcagtaca ttactggcct ggctgagcat ctgagccccc tgatgctgag cacctcttgg |
| 2101 | accagaatca ccctgtggaa cagggacctg gctcccactc ctggggccaa cctgtatggc |
| 2161 | agccacccct tctacctggc cctggaggat gggggctctg cccatggggt gttcctgctg |
| 2221 | aacagcaatg ccatggatgt ggtgctgcag ccctctcctg ccctgtcttg gagatctact |
| 2281 | gggggcatcc tggatgtgta tatcttcctg gggcctgagc ccaagtctgt ggtgcagcag |
| 2341 | tacctggatg tggtgggcta ccccttcatg cccccctact ggggcctggg cttccacctg |
| 2401 | tgcaggtggg gctactcttc tactgctatc accaggcagg tggtggagaa catgaccagg |
| 2461 | gctcacttcc ctctggatgt gcagtggaat gacctggact acatggactc taggagagac |
| 2521 | ttcactttta ataaggatgg gttcagggac tttcctgcca tggtgcagga gctgcatcag |
| 2581 | gggggcagga gatatatgat gattgtggac cctgctattt ctagctctgg ccctgctggc |
| 2641 | agctataggc cctatgatga ggggctgagg aggggggtgt tcatcactaa tgagactggc |
| 2701 | cagcccctga ttggcaaggt gtggcctggc tctactgcct tccctgattt caccaacccc |
| 2761 | actgccctgg cctggtggga ggatatggtg gctgagtttc atgaccaggt gccctttgat |
| 2821 | ggcatgtgga ttgacatgaa tgagcccagc aactttatca ggggctctga agatggctgc |
| 2881 | cccaacaatg agctggagaa ccccccctat gtgcctgggg tggtgggggg caccctgcag |
| 2941 | gctgccacca tctgtgccag cagccaccag ttcctgagca cccactataa cctgcacaac |
| 3001 | ctgtatggcc tgactgaggc cattgcctct cacagggccc tggtgaaggc tagggggact |
| 3061 | aggccctttg tgatcagcag gtctactttt gctggccatg gcaggtatgc tgggcactgg |
| 3121 | actggggatg tgtggtctag ctgggagcag ctggccagct ctgtgcctga gatcctgcag |
| 3181 | tttaatctgc tgggggtgcc cctggtgggg gctgatgtgt gtggcttcct gggcaatacc |
| 3241 | tctgaggagc tgtgtgtgag gtggactcag ctgggggctt tctacccctt catgagaaac |
| 3301 | cacaactctc tgctgagcct gccccaggag ccctattctt tttctgagcc tgcccagcag |
| 3361 | gctatgagga aggccctgac tctgaggtat gccctgctgc cccacctgta taccctgttc |
| 3421 | catcaggccc atgtggctgg ggagactgtg gccagacctc tgttcctgga gttccccaag |
| 3481 | gatagctcta cttggactgt ggaccaccag ctgctgtggg gggaggctct gctgatcacc |
| 3541 | cctgtgctgc aggctgggaa ggctgaggtg actggctatt tccccctggg cacctggtat |
| 3601 | gatctgcaga ctgtgcccat tgaggccctg ggctctctgc cccctccccc tgctgccccc |
| 3661 | agggagcctg ccatccactc tgagggccag tgggtgaccc tgcctgcccc tctggacact |
| 3721 | atcaatgtgc acctgagggc tggctacatc atccccctgc agggccctgg cctgactacc |
| 3781 | actgagtcta ggcagcagcc catggccctg gctgtggctc tgaccaaggg gggggaggcc |
| 3841 | aggggggagc tgttctggga tgatggggag tctctggagg tgctggagag gggggcctac |
| 3901 | acccaggtga tcttcctggc taggaataac accattgtca atgagctggt gagggtgacc |
| 3961 | tctgaggggg ctggcctgca gctgcagaag gtgactgtgc tgggggtggc tactgccccc |
| 4021 | cagcaggtgc tgagcaatgg ggtgcctgtg agcaacttca cctatagccc tgacaccaag |
| 4081 | gtgctggaca tctgtgtgag cctgctgatg ggggagcagt tcctggtgag ctggtgctaa |
| 4141 | tttaaatctc gagccgggcg gagtgtgtta gtctctccag agggaggctg gttccccagg |
| 4201 | gaagcagagc ctgtgtgcgg gcagcagctg tgtgcgggcc tgggggttgt taagtgcaat |
| 4261 | tatttttaat aaaaggggca tttggaaaaa aaaaaaaaag gtagcagtcg acagatgaat |
| 4321 | tctgcagatc tgtggcttct agctgcccgg gtggcatccc tgtgacccct ccccagtgcc |
| 4381 | tctcctggcc ctggaagttg ccactccagt gcccaccagc cttgtcctaa taaaattaag |
| 4441 | ttgcatcatt ttgtctgact aggtgtcctt ctataatatt atggggtgga ggggggtggt |
| 4501 | atggagcaag gggcaagttg ggaagacaac ctgtagggcc tgcggggtct attgggaacc |
| 4561 | aagctggagt gcagtggcac aatcttggct cactgcaatc tccgcctcct gggttcaagc |
| 4621 | gattctcctg cctcagcctc ccgagttgtt gggattccag gcatgcatga ccaggctcag |
| 4681 | ctaatttttg tttttttggt agagacgggg tttcaccata ttggccaggc tggtctccaa |
| 4741 | ctcctaatct caggtgatct acccaccttg gcctcccaaa ttgctgggat tacaggcgtg |
| 4801 | aaccactgct cccttccctg tccttctgat tttaaaaaaa agagaaatgt tctggcacct |
| 4861 | gcacttgcac tggggacagc ctattttgct agtttgtttt gtttcgtttt gttttgatgg |
| 4921 | agagcgtatg ttgtttaaac gcggccgcgt agataagtag catggcgggt taatcattaa |
| 4981 | ctacaaggaa cccctagtga tggagttggc cactccctct ctgcgcgctc gctcgctcac |
| 5041 | tgaggccggg cgaccaaagg tcgcccgacg cccgggcttt gcccgggcgg cctcagtgag |
| 5101 | cgagcgagcg cgcagttaat taaggcgccc taggccgacc cttagactct gtactcagtt |
| 5161 | ctataaacga gccattggat acgagatccg tagattgata agggacacgg aatatccccg |
| 5221 | gacgcaatag acaccggtgg acagcttggt atcctgagca cagtcgcgcg tccgaatcta |
| 5281 | gctctacttt agaggccccg gattctgatg gtcgtagacc gcagaaccga ttggggggat |
| 5341 | gagatctact agttatcagc acacaattgc ccattatacg cgcgtataat ggactattgt |
| 5401 | gtgctgatat agggataaca gggtaattct agagctagca tatggatcca tcgatttgat |
| 5461 | gcggtatttt ctccttacgc atctgtgcgg tatttcacac cgcatacgtc aaagcaacca |
| 5521 | tagtacgcgc cctgtagcgg cgcattaagc gcggcgggtg tggtggttac gcgcagcgtg |
| 5581 | accgctacac ttgccagcgc cctagcgccc gctcctttcg ctttcttccc ttcctttctc |
| 5641 | gccacgttcg ccggctttcc ccgtcaagct ctaaatcggg ggctcccttt agggttccga |
| 5701 | tttagtgctt tacggcacct cgaccccaaa aaacttgatt tgggtgatgg ttcacgtagt |
| 5761 | gggccatcgc cctgatagac ggtttttcgc cctttgacgt tggagtccac gttctttaat |
| 5821 | agtggactct tgttccaaac tggaacaaca ctcaactcta tctcgggcta ttcttttgat |
| 5881 | ttataaggga ttttgccgat ttcggtctat tggttaaaaa atgagctgat ttaacaaaaa |
| 5941 | tttaacgcga attttaacaa aatattaacg tttacaattt tatggtgcac tctcagtaca |
| 6001 | atctgctctg atgccgcata gttaagccag ccccgacacc cgccaacacc cgctgacgcg |
| 6061 | ccctgacggg cttgtctgct cccggcatcc gcttacagac aagctgtgac cgtctccggg |
| 6121 | agctgcatgt gtcagaggtt ttcaccgtca tcaccgaaac gcgcgagacg aaagggcctc |
| 6181 | gtgatacgcc tatttttata ggttaatgtc atgataataa tggtttctta gacgtcaggt |
| 6241 | ggcacttttc ggggaaatgt gcgcggaacc cctatttgtt tatttttcta aatacattca |
| 6301 | aatatgtatc cgctcatgag acaataaccc tgataaatgc ttcaataata ttgaaaaagg |
| 6361 | aagagtatga gccatattca acgggaaacg tcgaggccgc gattaaattc caacatggat |
| 6421 | gctgatttat atgggtataa atgggctcgc gataatgtcg ggcaatcagg tgcgacaatc |
| 6481 | tatcgcttgt atgggaagcc cgatgcgcca gagttgtttc tgaaacatgg caaaggtagc |
| 6541 | gttgccaatg atgttacaga tgagatggtc agactaaact ggctgacgga atttatgcct |
| 6601 | cttccgacca tcaagcattt tatccgtact cctgatgatg catggttact caccactgcg |
| 6661 | atccccggaa aaacagcatt ccaggtatta gaagaatatc ctgattcagg tgaaaatatt |
| 6721 | gttgatgcgc tggcagtgtt cctgcgccgg ttgcattcga ttcctgtttg taattgtcct |
| 6781 | tttaacagcg atcgcgtatt tcgtctcgct caggcgcaat cacgaatgaa taacggtttg |
| 6841 | gttgatgcga gtgattttga tgacgagcgt aatggctggc ctgttgaaca agtctggaaa |
| 6901 | gaaatgcata aacttttgcc attctcaccg gattcagtcg tcactcatgg tgatttctca |
| 6961 | cttgataacc ttatttttga cgaggggaaa ttaataggtt gtattgatgt tggacgagtc |
| 7021 | ggaatcgcag accgatacca ggatcttgcc atcctatgga actgcctcgg tgagttttct |
| 7081 | ccttcattac agaaacggct ttttcaaaaa tatggtattg ataatcctga tatgaataaa |
| 7141 | ttgcagtttc atttgatgct cgatgagttt ttctaagcgt ataatggtct agagctagca |
| 7201 | tatggatcca tcgattccat tatacgcctg tcagaccaag tttactcata tatactttag |
| 7261 | attgatttaa aacttcattt ttaatttaaa aggatctagg tgaagatcct ttttgataat |
| 7321 | ctcatgacca aaatccctta acgtgagttt tcgttccact gagcgtcaga ccccgtagaa |
| 7381 | aagatcaaag gatcttcttg agatcctttt tttctgcgcg taatctgctg cttgcaaaca |
| 7441 | aaaaaaccac cgctaccagc ggtggtttgt ttgccggatc aagagctacc aactcttttt |
| 7501 | ccgaaggtaa ctggcttcag cagagcgcag ataccaaata ctgttcttct agtgtagccg |
| 7561 | tagttaggcc accacttcaa gaactctgta gcaccgccta catacctcgc tctgctaatc |
| 7621 | ctgttaccag tggctgctgc cagtggcgat aagtcgtgtc ttaccgggtt ggactcaaga |
| 7681 | cgatagttac cggataaggc gcagcggtcg ggctgaacgg ggggttcgtg cacacagccc |
| 7741 | agcttggagc gaacgaccta caccgaactg agatacctac agcgtgagct atgagaaagc |
| 7801 | gccacgcttc ccgaagggag aaaggcggac aggtatccgg taagcggcag ggtcggaaca |
| 7861 | ggagagcgca cgagggagct tccaggggga aacgcctggt atctttatag tcctgtcggg |
| 7921 | tttcgccacc tctgacttga gcgtcgattt ttgtgatgct cgtcaggggg gcggagccta |
| 7981 | tggaaaaacg ccagcaacgc ggccttttta cggttcctgg ccttttgctg gccttttgct |
| 8041 | cacatgt |

| pP093_seq3-IL2wt_28-56 deletion (SEQ ID NO: 498) | |
|---|---|
| (SEQ ID NO: 498) | |
| 1 | tctagagcta gcatatggat ccatcgattt agggataaca gggtaattat cagcacacaa |
| 61 | ttgcccatta tacgcgcgta taatggacta ttgtgtgctg atatctgtac acttaagggc |
| 121 | tagatcttag cttacgtcac tagagggtcc acgtttagtt tttaagatcc attgatctcc |
| 181 | taaacgctgc aagattcgca acctggtata cttagcctag gcgctaggtc ctagtgcagc |
| 241 | gggacttttt ttctaaagtc gttgagagga ggagtcgtca gaccagatag ctttgatgtc |
| 301 | ctgatcggaa ggatcgttgg cccccctgca ggcagctgtt aattaactgc gcgctcgctc |
| 361 | gctcactgag gccgcccggg caaagcccgg gcgtcgggcg acctttggtc gcccggcctc |
| 421 | agtgagcgag cgagcgcgca gagagggagt ggccaactcc atcactaggg gttccttgta |
| 481 | gttaatgatt aacccgccat gctacttatc tacggcgcgc cacgcgttct aggagttaat |
| 541 | ttttaaaaag cagtcaaaag tccaagtgcc cttgcgagca tttactctct ctgtttgctc |
| 601 | tggttaataa tctcaggagc acaaacattc cttactagtt ctaggagtta atttttaaaa |
| 661 | agcagtcaaa agtccaagtg cccttgcgag catttactct ctctgtttgc tctggttaat |
| 721 | aatctcagga gcacaaacat tccttactag ttctagagcg gccgccagtg tgctggaatt |
| 781 | cggctttttt agggctggaa gctacctttg acatcatttc ctctgcgaat gcatgtataa |
| 841 | tttctacaga acctattaga aaggatcacc cagcctctgc ttttgtacaa ctttccctta |
| 901 | aaaaactgcc aatcccactg ctgtttggcc caatagtgag aactttttcc tgctgcctct |
| 961 | tggtgctttt gcctatggcc cctattctgc ctgctgaaga cactcttgcc agcatggact |
| 1021 | taaacccctc cagctctgac aatcctcttt ctcttttgtt ttacatgaag ggtctggcag |
| 1081 | ccaaagcaat cactcaaagt tcaaacctta tcattttttg ctttgttcct cttggccttg |
| 1141 | gttttgtaca tcagctttga aaataccatc ccagggttaa tgctggggtt aatttataac |
| 1201 | tgagagtgct ctagttctgc aatacaggac atgctataaa aatggaaaga tgttgctttc |
| 1261 | tgagagatca ggtggattct tgggcatttg cttaagcttg gtaccgccac catgggggtg |
| 1321 | aggcaccctc cctgttctca caggctgctg gctgtgtgtg ctctggtgtc tctggccact |
| 1381 | gctgccctgc tgtacagaat gcagctgctg tcctgcattg cactgtcact ggcactggtt |
| 1441 | accaactcac agcagggagc ttctagacct ggacctagag atgctcaggc tcatcctgga |
| 1501 | agacctagag ctgtgcctac acagtgtgat gtgcctccta attctagatt tgattgtgct |
| 1561 | cctgataagg ctataacaca ggagcagtgt gaggctagag gatgttgtta tattcctgct |
| 1621 | aagcagggac tgcagggagc tcagatggga cagccttggt gtttttttcc tccttcttat |
| 1681 | ccttcttata agctggagaa tctgtcttct tctgagatgg gatatacagc tacactgaca |
| 1741 | agaacaacac ctacattttt tcctaaggat attctgacac tgagactgga tgtgatgatg |
| 1801 | gagacagaga atagactgca ttttacaatt aaggatcctg ctaatagaag atatgaggtg |
| 1861 | cctctagaga cacctagagt gcattctaga gctccttctc ctctgtattc tgtggagttt |
| 1921 | tctgaggagc cttttggagt gattgtgcat agacagctgg atggaagagt gctgctgaat |
| 1981 | acaacagtgg ctcctctgtt ttttgctgat cagtttctgc agctgtctac atctctgcct |
| 2041 | tctcagtata ttacaggact ggctgagcat ctgtctcctc tgatgctgtc tacatcttgg |
| 2101 | acaagaatta cactgtggaa tagagatctg gctcctacac ctggagctaa tctgtatgga |
| 2161 | tctcatcctt tttatctggc tctggaggat ggaggatctg ctcatggagt gtttctgctg |
| 2221 | aattctaatg ctatggatgt agtgctgcag ccttctcctg ctctgtcttg gagatctaca |
| 2281 | ggaggaattc tggatgtgta tatttttcta ggacctgagc ctaagtctgt ggtgcagcag |
| 2341 | tatctggatg tagtgggata tccttttatg cctccttatt ggggactggg atttcatctg |
| 2401 | tgtagatggg gatattcttc tacagctatt acaagacagg tggttgagaa tatgacaaga |
| 2461 | gctcattttc ctctggatgt gcagtggaat gatctagatt atatggattc tagaagagat |
| 2521 | tttacattta ataaggatgg atttagagat tttcctgcta tggtgcagga gctgcatcag |
| 2581 | ggaggaagaa gatatatgat gattgtggat cctgctattt cttcttctgg acctgctgga |
| 2641 | tcttatagac cttatgatga gggactgaga agaggagtgt ttattacaaa tgagacagga |
| 2701 | cagcctctga ttggaaaggt gtggcctgga tctacagctt ttcctgattt tacaaatcct |
| 2761 | acagctctgg cttggtggga ggatatggtg gctgagtttc atgatcaggt gccttttgat |
| 2821 | ggaatgtgga ttgatatgaa tgagccttct aattttataa gaggatctga ggatggatgt |
| 2881 | cctaataatg agctagagaa tcctccttat gtgcctggag tagtgggagg aacactgcag |
| 2941 | gctgctacaa tttgtgcttc ttctcatcag tttctgtcta cacattataa tctgcataat |
| 3001 | ctgtatggac tgacagaggc tattgcttct catagagctc tggtgaaggc tagaggaaca |
| 3061 | agaccttttg tgatttctag atctacattt gctggacatg gaagatatgc tggacattgg |
| 3121 | acaggagatg tatggtcttc ttgggagcag ctagcttctt ctgtgcctga gattctgcag |
| 3181 | tttaatctgc taggagtgcc tctggtggga gctgatgtgt gtggatttct gggaaataca |
| 3241 | tctgaggagc tgtgtgtgag atggacacag ctgggagctt tttatccttt tatgagaaat |
| 3301 | cataattctc tgctgtctct gcctcaggag ccttattctt tttctgagcc tgctcagcag |
| 3361 | gctatgagaa aggctctgac actgagatat gctctgctgc ctcatctgta tacactgttt |
| 3421 | catcaggctc atgtggctgg agagacagta gctagacctc tgtttctgga gtttcctaag |
| 3481 | gattcttcta catggacagt tgatcatcag ctgctgtggg gagaggctct gctgattaca |
| 3541 | cctgtgctgc aggctggaaa ggctgaggtg acaggatatt ttcctctggg aacatggtat |
| 3601 | gatctgcaga cagtgcctat agaggctctg ggatctctgc ctcctcctcc tgctgctcct |
| 3661 | agagagcctg ctattcattc tgagggacag tgggtgacac tgcctgctcc tctggataca |
| 3721 | attaatgtgc atctgagagc tggatatatt attcctctgc agggacctgg actgacaaca |
| 3781 | acagagtcta gacagcagcc tatggctctg gctgtggctc tgacaaaggg aggagaggct |
| 3841 | agaggagagc tgttttggga tgatggagag tctctggagg tgctggagag aggagcttat |
| 3901 | acacaggtga tttttctggc tagaaataat acaattgtga atgagctggt gagagtgaca |
| 3961 | tctgagggag ctggactgca gctgcagaag gtgacagtgc tgggagtggc tacagctcct |
| 4021 | cagcaggtgc tgtctaatgg agtgcctgtg tctaatttta catattctcc tgatacaaag |
| 4081 | gtgctggata tttgtgtgtc tctgctgatg ggagagcagt ttctggtgtc ttggtgttaa |
| 4141 | tgatttaaat ctcgagccgg gcggagtgtg ttagtctctc cagagggagg ctggttcccc |
| 4201 | agggaagcag agcctgtgtg cgggcagcag ctgtgtgcgg gcctgggggt tgttaagtgc |
| 4261 | aattattttt aataaaaggg gcatttggaa aaaaaaaaaa aaggtagcag tcgacagatg |
| 4321 | aattctgcag atctgtggct tctagctgcc cgggtggcat ccctgtgacc cctccccagt |
| 4381 | gcctctcctg gccctggaag ttgccactcc agtgcccacc agccttgtcc taataaaatt |
| 4441 | aagttgcatc attttgtctg actaggtgtc cttctataat attatggggt ggaggggggt |
| 4501 | ggtatggagc aaggggcaag ttgggaagac aacctgtagg gcctgcgggg tctattggga |
| 4561 | accaagctgg agtgcagtgg cacaatcttg gctcactgca atctccgcct cctgggttca |
| 4621 | agcgattctc ctgcctcagc ctcccgagtt gttgggattc caggcatgca tgaccaggct |
| 4681 | cagctaattt ttgttttttt ggtagagacg gggtttcacc atattggcca ggctggtctc |
| 4741 | caactcctaa tctcaggtga tctacccacc ttggcctccc aaattgctgg gattacaggc |
| 4801 | gtgaaccact gctcccttcc ctgtccttct gattttaaaa aaaagagaaa tgttctggca |
| 4861 | cctgcacttg cactggggac agcctatttt gctagtttgt tttgtttcgt tttgttttga |
| 4921 | tggagagcgt atgttgttta aacgcggccg cgtagataag tagcatggcg ggttaatcat |
| 4981 | taactacaag gaacccctag tgatggagtt ggccactccc tctctgcgcg ctcgctcgct |
| 5041 | cactgaggcc gggcgaccaa aggtcgcccg acgcccgggc tttgcccggg cggcctcagt |
| 5101 | gagcgagcga gcgcgcagtt aattaaggcg ccctaggccg acccttagac tctgtactca |
| 5161 | gttctataaa cgagccattg gatacgagat ccgtagattg ataagggaca cggaatatcc |
| 5221 | ccggacgcaa tagacaccgg tggacagctt ggtatcctga gcacagtcgc gcgtccgaat |
| 5281 | ctagctctac tttagaggcc ccggattctg atggtcgtag accgcagaac cgattggggg |
| 5341 | gatgagatct actagttatc agcacacaat tgcccattat acgcgcgtat aatggactat |
| 5401 | tgtgtgctga tatagggata acagggtaat tctagagcta gcatatggat ccatcgattt |
| 5461 | gatgcggtat tttctcctta cgcatctgtg cggtatttca caccgcatac gtcaaagcaa |
| 5521 | ccatagtacg cgccctgtag cggcgcatta agcgcggcgg gtgtggtggt tacgcgcagc |
| 5581 | gtgaccgcta cacttgccag cgccctagcg cccgctcctt tcgctttctt cccttccttt |
| 5641 | ctcgccacgt tcgccggctt tccccgtcaa gctctaaatc gggggctccc tttagggttc |
| 5701 | cgatttagtg ctttacggca cctcgacccc aaaaaacttg atttgggtga tggttcacgt |
| 5761 | agtgggccat cgccctgata gacggttttt cgccctttga cgttggagtc cacgttcttt |
| 5821 | aatagtggac tcttgttcca aactggaaca acactcaact ctatctcggg ctattctttt |
| 5881 | gatttataag ggattttgcc gatttcggtc tattggttaa aaaatgagct gatttaacaa |
| 5941 | aaatttaacg cgaattttaa caaaatatta acgtttacaa ttttatggtg cactctcagt |
| 6001 | acaatctgct ctgatgccgc atagttaagc cagccccgac acccgccaac acccgctgac |
| 6061 | gcgccctgac gggcttgtct gctcccggca tccgcttaca gacaagctgt gaccgtctcc |
| 6121 | gggagctgca tgtgtcagag gttttcaccg tcatcaccga aacgcgcgag acgaaagggc |
| 6181 | ctcgtgatac gcctattttt ataggttaat gtcatgataa taatggtttc ttagacgtca |
| 6241 | ggtggcactt ttcggggaaa tgtgcgcgga acccctattt gtttattttt ctaaatacat |
| 6301 | tcaaatatgt atccgctcat gagacaataa ccctgataaa tgcttcaata atattgaaaa |
| 6361 | aggaagagta tgagccatat tcaacgggaa acgtcgaggc cgcgattaaa ttccaacatg |
| 6421 | gatgctgatt tatatgggta taaatgggct cgcgataatg tcgggcaatc aggtgcgaca |
| 6481 | atctatcgct tgtatgggaa gcccgatgcg ccagagttgt ttctgaaaca tggcaaaggt |
| 6541 | agcgttgcca atgatgttac agatgagatg gtcagactaa actggctgac ggaatttatg |
| 6601 | cctcttccga ccatcaagca ttttatccgt actcctgatg atgcatggtt actcaccact |
| 6661 | gcgatccccg gaaaaacagc attccaggta ttagaagaat atcctgattc aggtgaaaat |
| 6721 | attgttgatg cgctggcagt gttcctgcgc cggttgcatt cgattcctgt ttgtaattgt |
| 6781 | ccttttaaca gcgatcgcgt atttcgtctc gctcaggcgc aatcacgaat gaataacggt |
| 6841 | ttggttgatg cgagtgattt tgatgacgag cgtaatggct ggcctgttga acaagtctgg |
| 6901 | aaagaaatgc ataaactttt gccattctca ccggattcag tcgtcactca tggtgatttc |
| 6961 | tcacttgata accttatttt tgacgagggg aaattaatag gttgtattga tgttggacga |
| 7021 | gtcggaatcg cagaccgata ccaggatctt gccatcctat ggaactgcct cggtgagttt |
| 7081 | tctccttcat tacagaaacg gctttttcaa aaatatggta ttgataatcc tgatatgaat |
| 7141 | aaattgcagt ttcatttgat gctcgatgag tttttctaag cgtataatgg tctagagcta |
| 7201 | gcatatggat ccatcgattc cattatacgc ctgtcagacc aagtttactc atatatactt |
| 7261 | tagattgatt taaaacttca tttttaattt aaaaggatct aggtgaagat cctttttgat |
| 7321 | aatctcatga ccaaaatccc ttaacgtgag ttttcgttcc actgagcgtc agaccccgta |
| 7381 | gaaaagatca aaggatcttc ttgagatcct ttttttctgc gcgtaatctg ctgcttgcaa |
| 7441 | acaaaaaaac caccgctacc agcggtggtt tgtttgccgg atcaagagct accaactctt |
| 7501 | tttccgaagg taactggctt cagcagagcg cagataccaa atactgttct tctagtgtag |
| 7561 | ccgtagttag gccaccactt caagaactct gtagcaccgc ctacatacct cgctctgcta |
| 7621 | atcctgttac cagtggctgc tgccagtggc gataagtcgt gtcttaccgg gttggactca |
| 7681 | agacgatagt taccggataa ggcgcagcgg tcgggctgaa cggggggttc gtgcacacag |
| 7741 | cccagcttgg agcgaacgac ctacaccgaa ctgagatacc tacagcgtga gctatgagaa |
| 7801 | agcgccacgc ttcccgaagg gagaaaggcg gacaggtatc cggtaagcgg cagggtcgga |
| 7861 | acaggagagc gcacgaggga gcttccaggg ggaaacgcct ggtatcttta tagtcctgtc |
| 7921 | gggtttcgcc acctctgact tgagcgtcga tttttgtgat gctcgtcagg ggggcggagc |
| 7981 | ctatggaaaa acgccagcaa cgcggccttt ttacggttcc tggccttttg ctggcctttt |
| 8041 | gctcacatgt |

| pP094_seq3-201Ig_28-56 deletion (SEQ ID NO: 499) | |
|---|---|
| (SEQ ID NO: 499) | |
| 1 | tctagagcta gcatatggat ccatcgattt agggataaca gggtaattat cagcacacaa |
| 61 | ttgcccatta tacgcgcgta taatggacta ttgtgtgctg atatctgtac acttaagggc |
| 121 | tagatcttag cttacgtcac tagagggtcc acgtttagtt tttaagatcc attgatctcc |
| 181 | taaacgctgc aagattcgca acctggtata cttagcctag gcgctaggtc ctagtgcagc |
| 241 | gggacttttt ttctaaagtc gttgagagga ggagtcgtca gaccagatag ctttgatgtc |
| 301 | ctgatcggaa ggatcgttgg cccccctgca ggcagctgtt aattaactgc gcgctcgctc |
| 361 | gctcactgag gccgcccggg caaagcccgg gcgtcgggcg acctttggtc gcccggcctc |
| 421 | agtgagcgag cgagcgcgca gagagggagt ggccaactcc atcactaggg gttccttgta |
| 481 | gttaatgatt aacccgccat gctacttatc tacggcgcgc cacgcgttct aggagttaat |
| 541 | ttttaaaaag cagtcaaaag tccaagtgcc cttgcgagca tttactctct ctgtttgctc |
| 601 | tggttaataa tctcaggagc acaaacattc cttactagtt ctaggagtta atttttaaaa |
| 661 | agcagtcaaa agtccaagtg cccttgcgag catttactct ctctgtttgc tctggttaat |
| 721 | aatctcagga gcacaaacat tccttactag ttctagagcg gccgccagtg tgctggaatt |
| 781 | cggctttttt agggctggaa gctacctttg acatcatttc ctctgcgaat gcatgtataa |
| 841 | tttctacaga acctattaga aaggatcacc cagcctctgc ttttgtacaa ctttccctta |
| 901 | aaaaactgcc aatcccactg ctgtttggcc caatagtgag aactttttcc tgctgcctct |
| 961 | tggtgctttt gcctatggcc cctattctgc ctgctgaaga cactcttgcc agcatggact |
| 1021 | taaacccctc cagctctgac aatcctcttt ctcttttgtt ttacatgaag ggtctggcag |
| 1081 | ccaaagcaat cactcaaagt tcaaacctta tcattttttg ctttgttcct cttggccttg |
| 1141 | gttttgtaca tcagctttga aaataccatc ccagggttaa tgctggggtt aatttataac |
| 1201 | tgagagtgct ctagttctgc aatacaggac atgctataaa aatggaaaga tgttgctttc |
| 1261 | tgagagatca ggtggattct tgggcatttg cttaagcttg gtaccgccac catgggggtg |
| 1321 | aggcaccctc cctgttctca caggctgctg gctgtgtgtg ctctggtgtc tctggccact |
| 1381 | gctgccctgc tggagtttgg gctgagctgg gtctttctgg tggccctgct gaagggagtc |
| 1441 | cagtgtgagc agcagggagc ttctagacct ggacctagag atgctcaggc tcatcctgga |
| 1501 | agacctagag ctgtgcctac acagtgtgat gtgcctccta attctagatt tgattgtgct |
| 1561 | cctgataagg ctataacaca ggagcagtgt gaggctagag gatgttgtta tattcctgct |
| 1621 | aagcagggac tgcagggagc tcagatggga cagccttggt gtttttttcc tccttcttat |
| 1681 | ccttcttata agctggagaa tctgtcttct tctgagatgg gatatacagc tacactgaca |
| 1741 | agaacaacac ctacattttt tcctaaggat attctgacac tgagactgga tgtgatgatg |
| 1801 | gagacagaga atagactgca ttttacaatt aaggatcctg ctaatagaag atatgaggtg |
| 1861 | cctctagaga cacctagagt gcattctaga gctccttctc ctctgtattc tgtggagttt |
| 1921 | tctgaggagc cttttggagt gattgtgcat agacagctgg atggaagagt gctgctgaat |
| 1981 | acaacagtgg ctcctctgtt ttttgctgat cagtttctgc agctgtctac atctctgcct |
| 2041 | tctcagtata ttacaggact ggctgagcat ctgtctcctc tgatgctgtc tacatcttgg |
| 2101 | acaagaatta cactgtggaa tagagatctg gctcctacac ctggagctaa tctgtatgga |
| 2161 | tctcatcctt tttatctggc tctggaggat ggaggatctg ctcatggagt gtttctgctg |
| 2221 | aattctaatg ctatggatgt agtgctgcag ccttctcctg ctctgtcttg gagatctaca |
| 2281 | ggaggaattc tggatgtgta tatttttcta ggacctgagc ctaagtctgt ggtgcagcag |
| 2341 | tatctggatg tagtgggata tccttttatg cctccttatt ggggactggg atttcatctg |
| 2401 | tgtagatggg gatattcttc tacagctatt acaagacagg tggttgagaa tatgacaaga |
| 2461 | gctcattttc ctctggatgt gcagtggaat gatctagatt atatggattc tagaagagat |
| 2521 | tttacattta ataaggatgg atttagagat tttcctgcta tggtgcagga gctgcatcag |
| 2581 | ggaggaagaa gatatatgat gattgtggat cctgctattt cttcttctgg acctgctgga |
| 2641 | tcttatagac cttatgatga gggactgaga agaggagtgt ttattacaaa tgagacagga |
| 2701 | cagcctctga ttggaaaggt gtggcctgga tctacagctt ttcctgattt tacaaatcct |
| 2761 | acagctctgg cttggtggga ggatatggtg gctgagtttc atgatcaggt gccttttgat |
| 2821 | ggaatgtgga ttgatatgaa tgagccttct aattttataa gaggatctga ggatggatgt |
| 2881 | cctaataatg agctagagaa tcctccttat gtgcctggag tagtgggagg aacactgcag |
| 2941 | gctgctacaa tttgtgcttc ttctcatcag tttctgtcta cacattataa tctgcataat |
| 3001 | ctgtatggac tgacagaggc tattgcttct catagagctc tggtgaaggc tagaggaaca |
| 3061 | agaccttttg tgatttctag atctacattt gctggacatg gaagatatgc tggacattgg |
| 3121 | acaggagatg tatggtcttc ttgggagcag ctagcttctt ctgtgcctga gattctgcag |
| 3181 | tttaatctgc taggagtgcc tctggtggga gctgatgtgt gtggatttct gggaaataca |
| 3241 | tctgaggagc tgtgtgtgag atggacacag ctgggagctt tttatccttt tatgagaaat |
| 3301 | cataattctc tgctgtctct gcctcaggag ccttattctt tttctgagcc tgctcagcag |
| 3361 | gctatgagaa aggctctgac actgagatat gctctgctgc ctcatctgta tacactgttt |
| 3421 | catcaggctc atgtggctgg agagacagta gctagacctc tgtttctgga gtttcctaag |
| 3481 | gattcttcta catggacagt tgatcatcag ctgctgtggg gagaggctct gctgattaca |
| 3541 | cctgtgctgc aggctggaaa ggctgaggtg acaggatatt ttcctctggg aacatggtat |
| 3601 | gatctgcaga cagtgcctat agaggctctg ggatctctgc ctcctcctcc tgctgctcct |
| 3661 | agagagcctg ctattcattc tgagggacag tgggtgacac tgcctgctcc tctggataca |
| 3721 | attaatgtgc atctgagagc tggatatatt attcctctgc agggacctgg actgacaaca |
| 3781 | acagagtcta gacagcagcc tatggctctg gctgtggctc tgacaaaggg aggagaggct |
| 3841 | agaggagagc tgttttggga tgatggagag tctctggagg tgctggagag aggagcttat |
| 3901 | acacaggtga tttttctggc tagaaataat acaattgtga atgagctggt gagagtgaca |
| 3961 | tctgagggag ctggactgca gctgcagaag gtgacagtgc tgggagtggc tacagctcct |
| 4021 | cagcaggtgc tgtctaatgg agtgcctgtg tctaatttta catattctcc tgatacaaag |
| 4081 | gtgctggata tttgtgtgtc tctgctgatg ggagagcagt ttctggtgtc ttggtgttaa |
| 4141 | tgatttaaat ctcgagccgg gcggagtgtg ttagtctctc cagagggagg ctggttcccc |
| 4201 | agggaagcag agcctgtgtg cgggcagcag ctgtgtgcgg gcctgggggt tgttaagtgc |
| 4261 | aattattttt aataaaaggg gcatttggaa aaaaaaaaaa aaggtagcag tcgacagatg |
| 4321 | aattctgcag atctgtggct tctagctgcc cgggtggcat ccctgtgacc cctccccagt |
| 4381 | gcctctcctg gccctggaag ttgccactcc agtgcccacc agccttgtcc taataaaatt |
| 4441 | aagttgcatc attttgtctg actaggtgtc cttctataat attatggggt ggaggggggt |
| 4501 | ggtatggagc aaggggcaag ttgggaagac aacctgtagg gcctgcgggg tctattggga |
| 4561 | accaagctgg agtgcagtgg cacaatcttg gctcactgca atctccgcct cctgggttca |
| 4621 | agcgattctc ctgcctcagc ctcccgagtt gttgggattc caggcatgca tgaccaggct |
| 4681 | cagctaattt ttgttttttt ggtagagacg gggtttcacc atattggcca ggctggtctc |
| 4741 | caactcctaa tctcaggtga tctacccacc ttggcctccc aaattgctgg gattacaggc |
| 4801 | gtgaaccact gctcccttcc ctgtccttct gattttaaaa aaaagagaaa tgttctggca |
| 4861 | cctgcacttg cactggggac agcctatttt gctagtttgt tttgtttcgt tttgttttga |
| 4921 | tggagagcgt atgttgttta aacgcggccg cgtagataag tagcatggcg ggttaatcat |
| 4981 | taactacaag gaacccctag tgatggagtt ggccactccc tctctgcgcg ctcgctcgct |
| 5041 | cactgaggcc gggcgaccaa aggtcgcccg acgcccgggc tttgcccggg cggcctcagt |
| 5101 | gagcgagcga gcgcgcagtt aattaaggcg ccctaggccg acccttagac tctgtactca |
| 5161 | gttctataaa cgagccattg gatacgagat ccgtagattg ataagggaca cggaatatcc |
| 5221 | ccggacgcaa tagacaccgg tggacagctt ggtatcctga gcacagtcgc gcgtccgaat |
| 5281 | ctagctctac tttagaggcc ccggattctg atggtcgtag accgcagaac cgattggggg |
| 5341 | gatgagatct actagttatc agcacacaat tgcccattat acgcgcgtat aatggactat |
| 5401 | tgtgtgctga tatagggata acagggtaat tctagagcta gcatatggat ccatcgattt |
| 5461 | gatgcggtat tttctcctta cgcatctgtg cggtatttca caccgcatac gtcaaagcaa |
| 5521 | ccatagtacg cgccctgtag cggcgcatta agcgcggcgg gtgtggtggt tacgcgcagc |
| 5581 | gtgaccgcta cacttgccag cgccctagcg cccgctcctt tcgctttctt cccttccttt |
| 5641 | ctcgccacgt tcgccggctt tccccgtcaa gctctaaatc gggggctccc tttagggttc |
| 5701 | cgatttagtg ctttacggca cctcgacccc aaaaaacttg atttgggtga tggttcacgt |
| 5761 | agtgggccat cgccctgata gacggttttt cgccctttga cgttggagtc cacgttcttt |
| 5821 | aatagtggac tcttgttcca aactggaaca acactcaact ctatctcggg ctattctttt |
| 5881 | gatttataag ggattttgcc gatttcggtc tattggttaa aaaatgagct gatttaacaa |
| 5941 | aaatttaacg cgaattttaa caaaatatta acgtttacaa ttttatggtg cactctcagt |
| 6001 | acaatctgct ctgatgccgc atagttaagc cagccccgac acccgccaac acccgctgac |
| 6061 | gcgccctgac gggcttgtct gctcccggca tccgcttaca gacaagctgt gaccgtctcc |
| 6121 | gggagctgca tgtgtcagag gttttcaccg tcatcaccga aacgcgcgag acgaaagggc |
| 6181 | ctcgtgatac gcctattttt ataggttaat gtcatgataa taatggtttc ttagacgtca |
| 6241 | ggtggcactt ttcggggaaa tgtgcgcgga acccctattt gtttattttt ctaaatacat |
| 6301 | tcaaatatgt atccgctcat gagacaataa ccctgataaa tgcttcaata atattgaaaa |
| 6361 | aggaagagta tgagccatat tcaacgggaa acgtcgaggc cgcgattaaa ttccaacatg |
| 6421 | gatgctgatt tatatgggta taaatgggct cgcgataatg tcgggcaatc aggtgcgaca |
| 6481 | atctatcgct tgtatgggaa gcccgatgcg ccagagttgt ttctgaaaca tggcaaaggt |
| 6541 | agcgttgcca atgatgttac agatgagatg gtcagactaa actggctgac ggaatttatg |
| 6601 | cctcttccga ccatcaagca ttttatccgt actcctgatg atgcatggtt actcaccact |
| 6661 | gcgatccccg gaaaaacagc attccaggta ttagaagaat atcctgattc aggtgaaaat |
| 6721 | attgttgatg cgctggcagt gttcctgcgc cggttgcatt cgattcctgt ttgtaattgt |
| 6781 | ccttttaaca gcgatcgcgt atttcgtctc gctcaggcgc aatcacgaat gaataacggt |
| 6841 | ttggttgatg cgagtgattt tgatgacgag cgtaatggct ggcctgttga acaagtctgg |
| 6901 | aaagaaatgc ataaactttt gccattctca ccggattcag tcgtcactca tggtgatttc |
| 6961 | tcacttgata accttatttt tgacgagggg aaattaatag gttgtattga tgttggacga |
| 7021 | gtcggaatcg cagaccgata ccaggatctt gccatcctat ggaactgcct cggtgagttt |
| 7081 | tctccttcat tacagaaacg gctttttcaa aaatatggta ttgataatcc tgatatgaat |
| 7141 | aaattgcagt ttcatttgat gctcgatgag tttttctaag cgtataatgg tctagagcta |
| 7201 | gcatatggat ccatcgattc cattatacgc ctgtcagacc aagtttactc atatatactt |
| 7261 | tagattgatt taaaacttca tttttaattt aaaaggatct aggtgaagat cctttttgat |
| 7321 | aatctcatga ccaaaatccc ttaacgtgag ttttcgttcc actgagcgtc agaccccgta |
| 7381 | gaaaagatca aaggatcttc ttgagatcct ttttttctgc gcgtaatctg ctgcttgcaa |
| 7441 | acaaaaaaac caccgctacc agcggtggtt tgtttgccgg atcaagagct accaactctt |
| 7501 | tttccgaagg taactggctt cagcagagcg cagataccaa atactgttct tctagtgtag |
| 7561 | ccgtagttag gccaccactt caagaactct gtagcaccgc ctacatacct cgctctgcta |
| 7621 | atcctgttac cagtggctgc tgccagtggc gataagtcgt gtcttaccgg gttggactca |
| 7681 | agacgatagt taccggataa ggcgcagcgg tcgggctgaa cggggggttc gtgcacacag |
| 7741 | cccagcttgg agcgaacgac ctacaccgaa ctgagatacc tacagcgtga gctatgagaa |
| 7801 | agcgccacgc ttcccgaagg gagaaaggcg gacaggtatc cggtaagcgg cagggtcgga |
| 7861 | acaggagagc gcacgaggga gcttccaggg ggaaacgcct ggtatcttta tagtcctgtc |
| 7921 | gggtttcgcc acctctgact tgagcgtcga tttttgtgat gctcgtcagg ggggcggagc |
| 7981 | ctatggaaaa acgccagcaa cgcggccttt ttacggttcc tggccttttg ctggcctttt |
| 8041 | gctcacatgt |

| pP098_seq100_25-56 deletion (SEQ ID NO: 500) | |
|---|---|
| (SEQ ID NO: 500) | |
| 1 | tctagagcta gcatatggat ccatcgattt agggataaca gggtaattat cagcacacaa |
| 61 | ttgcccatta tacgcgcgta taatggacta ttgtgtgctg atatctgtac acttaagggc |
| 121 | tagatcttag cttacgtcac tagagggtcc acgtttagtt tttaagatcc attgatctcc |
| 181 | taaacgctgc aagattcgca acctggtata cttagcctag gcgctaggtc ctagtgcagc |
| 241 | gggacttttt ttctaaagtc gttgagagga ggagtcgtca gaccagatag ctttgatgtc |
| 301 | ctgatcggaa ggatcgttgg cccccctgca ggcagctgtt aattaactgc gcgctcgctc |
| 361 | gctcactgag gccgcccggg caaagcccgg gcgtcgggcg acctttggtc gcccggcctc |
| 421 | agtgagcgag cgagcgcgca gagagggagt ggccaactcc atcactaggg gttccttgta |
| 481 | gttaatgatt aacccgccat gctacttatc tacggcgcgc cacgcgttct aggagttaat |
| 541 | ttttaaaaag cagtcaaaag tccaagtgcc cttgcgagca tttactctct ctgtttgctc |
| 601 | tggttaataa tctcaggagc acaaacattc cttactagtt ctaggagtta atttttaaaa |
| 661 | agcagtcaaa agtccaagtg cccttgcgag catttactct ctctgtttgc tctggttaat |
| 721 | aatctcagga gcacaaacat tccttactag ttctagagcg gccgccagtg tgctggaatt |
| 781 | cggctttttt agggctggaa gctacctttg acatcatttc ctctgcgaat gcatgtataa |
| 841 | tttctacaga acctattaga aaggatcacc cagcctctgc ttttgtacaa ctttccctta |
| 901 | aaaaactgcc aatcccactg ctgtttggcc caatagtgag aactttttcc tgctgcctct |
| 961 | tggtgctttt gcctatggcc cctattctgc ctgctgaaga cactcttgcc agcatggact |
| 1021 | taaacccctc cagctctgac aatcctcttt ctcttttgtt ttacatgaag ggtctggcag |
| 1081 | ccaaagcaat cactcaaagt tcaaacctta tcattttttg ctttgttcct cttggccttg |
| 1141 | gttttgtaca tcagctttga aaataccatc ccagggttaa tgctggggtt aatttataac |
| 1201 | tgagagtgct ctagttctgc aatacaggac atgctataaa aatggaaaga tgttgctttc |
| 1261 | tgagagatca ggtggattct tgggcatttg cttaagcttg gtaccgccac catgggggtg |
| 1321 | aggcaccctc cctgttctca caggctgctg gctgtgtgtg ctctggtgtc tctggccact |
| 1381 | gctcagcagg gggccagcag gccaggcccc agggatgccc aggcccaccc tggcagaccc |
| 1441 | agggctgtgc ccacccagtg tgatgtgcct cccaacagca gatttgactg tgcccctgat |
| 1501 | aaggccatta ctcaggaaca gtgtgaggcc aggggctgct gctatatccc tgccaagcag |
| 1561 | ggcctgcagg gggcccagat ggggcagccc tggtgcttct tcccccccag ctacccctct |
| 1621 | tataagctgg agaatctgag cagctctgaa atgggctaca ctgccactct gaccaggact |
| 1681 | acccccacct tcttccccaa ggatattctg actctgaggc tggatgtgat gatggagact |
| 1741 | gagaacaggc tgcacttcac catcaaggac cctgccaaca ggaggtatga ggtgcccctg |
| 1801 | gaaactccca gggtgcactc tagggccccc agccccctgt attctgtgga gttctctgag |
| 1861 | gagccttttg gggtcattgt ccacaggcag ctggatggca gggtgctgct gaatactact |
| 1921 | gtggcccctc tgttctttgc tgaccagttc ctgcagctga gcaccagcct gccttctcag |
| 1981 | tacattactg gcctggctga gcatctgagc cccctgatgc tgagcacctc ttggaccaga |
| 2041 | atcaccctgt ggaacaggga cctggctccc actcctgggg ccaacctgta tggcagccac |
| 2101 | cccttctacc tggccctgga ggatgggggc tctgcccatg gggtgttcct gctgaacagc |
| 2161 | aatgccatgg atgtggtgct gcagccctct cctgccctgt cttggagatc tactgggggc |
| 2221 | atcctggatg tgtatatctt cctggggcct gagcccaagt ctgtggtgca gcagtacctg |
| 2281 | gatgtggtgg gctacccctt catgcccccc tactggggcc tgggcttcca cctgtgcagg |
| 2341 | tggggctact cttctactgc tatcaccagg caggtggtgg agaacatgac cagggctcac |
| 2401 | ttccctctgg atgtgcagtg gaatgacctg gactacatgg actctaggag agacttcact |
| 2461 | tttaataagg atgggttcag ggactttcct gccatggtgc aggagctgca tcaggggggc |
| 2521 | aggagatata tgatgattgt ggaccctgct atttctagct ctggccctgc tggcagctat |
| 2581 | aggccctatg atgaggggct gaggaggggg gtgttcatca ctaatgagac tggccagccc |
| 2641 | ctgattggca aggtgtggcc tggctctact gccttccctg atttcaccaa ccccactgcc |
| 2701 | ctggcctggt gggaggatat ggtggctgag tttcatgacc aggtgccctt tgatggcatg |
| 2761 | tggattgaca tgaatgagcc cagcaacttt atcaggggct ctgaagatgg ctgccccaac |
| 2821 | aatgagctgg agaacccccc ctatgtgcct ggggtggtgg ggggcaccct gcaggctgcc |
| 2881 | accatctgtg ccagcagcca ccagttcctg agcacccact ataacctgca caacctgtat |
| 2941 | ggcctgactg aggccattgc ctctcacagg gccctggtga aggctagggg gactaggccc |
| 3001 | tttgtgatca gcaggtctac ttttgctggc catggcaggt atgctgggca ctggactggg |
| 3061 | gatgtgtggt ctagctggga gcagctggcc agctctgtgc ctgagatcct gcagtttaat |
| 3121 | ctgctggggg tgcccctggt gggggctgat gtgtgtggct tcctgggcaa tacctctgag |
| 3181 | gagctgtgtg tgaggtggac tcagctgggg gctttctacc ccttcatgag aaaccacaac |
| 3241 | tctctgctga gcctgcccca ggagccctat tctttttctg agcctgccca gcaggctatg |
| 3301 | aggaaggccc tgactctgag gtatgccctg ctgccccacc tgtataccct gttccatcag |
| 3361 | gcccatgtgg ctggggagac tgtggccaga cctctgttcc tggagttccc caaggatagc |
| 3421 | tctacttgga ctgtggacca ccagctgctg tggggggagg ctctgctgat cacccctgtg |
| 3481 | ctgcaggctg ggaaggctga ggtgactggc tatttccccc tgggcacctg gtatgatctg |
| 3541 | cagactgtgc ccattgaggc cctgggctct ctgccccctc cccctgctgc ccccagggag |
| 3601 | cctgccatcc actctgaggg ccagtgggtg accctgcctg cccctctgga cactatcaat |
| 3661 | gtgcacctga gggctggcta catcatcccc ctgcagggcc ctggcctgac taccactgag |
| 3721 | tctaggcagc agcccatggc cctggctgtg gctctgacca agggggggga ggccaggggg |
| 3781 | gagctgttct gggatgatgg ggagtctctg gaggtgctgg agaggggggc ctacacccag |
| 3841 | gtgatcttcc tggctaggaa taacaccatt gtcaatgagc tggtgagggt gacctctgag |
| 3901 | ggggctggcc tgcagctgca gaaggtgact gtgctggggg tggctactgc cccccagcag |
| 3961 | gtgctgagca atggggtgcc tgtgagcaac ttcacctata gccctgacac caaggtgctg |
| 4021 | gacatctgtg tgagcctgct gatgggggag cagttcctgg tgagctggtg ctaatttaaa |
| 4081 | tctcgagccg ggcggagtgt gttagtctct ccagagggag gctggttccc cagggaagca |
| 4141 | gagcctgtgt gcgggcagca gctgtgtgcg ggcctggggg ttgttaagtg caattatttt |
| 4201 | taataaaagg ggcatttgga aaaaaaaaaa aaaggtagca gtcgacagat gaattctgca |
| 4261 | gatctgtggc ttctagctgc ccgggtggca tccctgtgac ccctccccag tgcctctcct |
| 4321 | ggccctggaa gttgccactc cagtgcccac cagccttgtc ctaataaaat taagttgcat |
| 4381 | cattttgtct gactaggtgt ccttctataa tattatgggg tggagggggg tggtatggag |
| 4441 | caaggggcaa gttgggaaga caacctgtag ggcctgcggg gtctattggg aaccaagctg |
| 4501 | gagtgcagtg gcacaatctt ggctcactgc aatctccgcc tcctgggttc aagcgattct |
| 4561 | cctgcctcag cctcccgagt tgttgggatt ccaggcatgc atgaccaggc tcagctaatt |
| 4621 | tttgtttttt tggtagagac ggggtttcac catattggcc aggctggtct ccaactccta |
| 4681 | atctcaggtg atctacccac cttggcctcc caaattgctg ggattacagg cgtgaaccac |
| 4741 | tgctcccttc cctgtccttc tgattttaaa aaaaagagaa atgttctggc acctgcactt |
| 4801 | gcactgggga cagcctattt tgctagtttg ttttgtttcg ttttgttttg atggagagcg |
| 4861 | tatgttgttt aaacgcggcc gcgtagataa gtagcatggc gggttaatca ttaactacaa |
| 4921 | ggaaccccta gtgatggagt tggccactcc ctctctgcgc gctcgctcgc tcactgaggc |
| 4981 | cgggcgacca aaggtcgccc gacgcccggg ctttgcccgg gcggcctcag tgagcgagcg |
| 5041 | agcgcgcagt taattaaggc gccctaggcc gacccttaga ctctgtactc agttctataa |
| 5101 | acgagccatt ggatacgaga tccgtagatt gataagggac acggaatatc cccggacgca |
| 5161 | atagacaccg gtggacagct tggtatcctg agcacagtcg cgcgtccgaa tctagctcta |
| 5221 | ctttagaggc cccggattct gatggtcgta gaccgcagaa ccgattgggg ggatgagatc |
| 5281 | tactagttat cagcacacaa ttgcccatta tacgcgcgta taatggacta ttgtgtgctg |
| 5341 | atatagggat aacagggtaa ttctagagct agcatatgga tccatcgatt tgatgcggta |
| 5401 | ttttctcctt acgcatctgt gcggtatttc acaccgcata cgtcaaagca accatagtac |
| 5461 | gcgccctgta gcggcgcatt aagcgcggcg ggtgtggtgg ttacgcgcag cgtgaccgct |
| 5521 | acacttgcca gcgccctagc gcccgctcct ttcgctttct tcccttcctt tctcgccacg |
| 5581 | ttcgccggct ttccccgtca agctctaaat cgggggctcc ctttagggtt ccgatttagt |
| 5641 | gctttacggc acctcgaccc caaaaaactt gatttgggtg atggttcacg tagtgggcca |
| 5701 | tcgccctgat agacggtttt tcgccctttg acgttggagt ccacgttctt taatagtgga |
| 5761 | ctcttgttcc aaactggaac aacactcaac tctatctcgg gctattcttt tgatttataa |
| 5821 | gggattttgc cgatttcggt ctattggtta aaaaatgagc tgatttaaca aaaatttaac |
| 5881 | gcgaatttta acaaaatatt aacgtttaca attttatggt gcactctcag tacaatctgc |
| 5941 | tctgatgccg catagttaag ccagccccga cacccgccaa cacccgctga cgcgccctga |
| 6001 | cgggcttgtc tgctcccggc atccgcttac agacaagctg tgaccgtctc cgggagctgc |
| 6061 | atgtgtcaga ggttttcacc gtcatcaccg aaacgcgcga gacgaaaggg cctcgtgata |
| 6121 | cgcctatttt tataggttaa tgtcatgata ataatggttt cttagacgtc aggtggcact |
| 6181 | tttcggggaa atgtgcgcgg aacccctatt tgtttatttt tctaaataca ttcaaatatg |
| 6241 | tatccgctca tgagacaata accctgataa atgcttcaat aatattgaaa aaggaagagt |
| 6301 | atgagccata ttcaacggga aacgtcgagg ccgcgattaa attccaacat ggatgctgat |
| 6361 | ttatatgggt ataaatgggc tcgcgataat gtcgggcaat caggtgcgac aatctatcgc |
| 6421 | ttgtatggga agcccgatgc gccagagttg tttctgaaac atggcaaagg tagcgttgcc |
| 6481 | aatgatgtta cagatgagat ggtcagacta aactggctga cggaatttat gcctcttccg |
| 6541 | accatcaagc attttatccg tactcctgat gatgcatggt tactcaccac tgcgatcccc |
| 6601 | ggaaaaacag cattccaggt attagaagaa tatcctgatt caggtgaaaa tattgttgat |
| 6661 | gcgctggcag tgttcctgcg ccggttgcat tcgattcctg tttgtaattg tccttttaac |
| 6721 | agcgatcgcg tatttcgtct cgctcaggcg caatcacgaa tgaataacgg tttggttgat |
| 6781 | gcgagtgatt ttgatgacga gcgtaatggc tggcctgttg aacaagtctg gaaagaaatg |
| 6841 | cataaacttt tgccattctc accggattca gtcgtcactc atggtgattt ctcacttgat |
| 6901 | aaccttattt ttgacgaggg gaaattaata ggttgtattg atgttggacg agtcggaatc |
| 6961 | gcagaccgat accaggatct tgccatccta tggaactgcc tcggtgagtt ttctccttca |
| 7021 | ttacagaaac ggctttttca aaaatatggt attgataatc ctgatatgaa taaattgcag |
| 7081 | tttcatttga tgctcgatga gtttttctaa gcgtataatg gtctagagct agcatatgga |
| 7141 | tccatcgatt ccattatacg cctgtcagac caagtttact catatatact ttagattgat |
| 7201 | ttaaaacttc atttttaatt taaaaggatc taggtgaaga tcctttttga taatctcatg |
| 7261 | accaaaatcc cttaacgtga gttttcgttc cactgagcgt cagaccccgt agaaaagatc |
| 7321 | aaaggatctt cttgagatcc tttttttctg cgcgtaatct gctgcttgca aacaaaaaaa |
| 7381 | ccaccgctac cagcggtggt ttgtttgccg gatcaagagc taccaactct ttttccgaag |
| 7441 | gtaactggct tcagcagagc gcagatacca aatactgttc ttctagtgta gccgtagtta |
| 7501 | ggccaccact tcaagaactc tgtagcaccg cctacatacc tcgctctgct aatcctgtta |
| 7561 | ccagtggctg ctgccagtgg cgataagtcg tgtcttaccg ggttggactc aagacgatag |
| 7621 | ttaccggata aggcgcagcg gtcgggctga acggggggtt cgtgcacaca gcccagcttg |
| 7681 | gagcgaacga cctacaccga actgagatac ctacagcgtg agctatgaga aagcgccacg |
| 7741 | cttcccgaag ggagaaaggc ggacaggtat ccggtaagcg gcagggtcgg aacaggagag |
| 7801 | cgcacgaggg agcttccagg gggaaacgcc tggtatcttt atagtcctgt cgggtttcgc |
| 7861 | cacctctgac ttgagcgtcg atttttgtga tgctcgtcag gggggcggag cctatggaaa |
| 7921 | aacgccagca acgcggcctt tttacggttc ctggcctttt gctggccttt tgctcacatg |
| 7981 | t |

| pP099_seq3_25-56 deletion (SEQ ID NO: 501) | |
|---|---|
| (SEQ ID NO: 501) | |
| 1 | tctagagcta gcatatggat ccatcgattt agggataaca gggtaattat cagcacacaa |
| 61 | ttgcccatta tacgcgcgta taatggacta ttgtgtgctg atatctgtac acttaagggc |
| 121 | tagatcttag cttacgtcac tagagggtcc acgtttagtt tttaagatcc attgatctcc |
| 181 | taaacgctgc aagattcgca acctggtata cttagcctag gcgctaggtc ctagtgcagc |
| 241 | gggacttttt ttctaaagtc gttgagagga ggagtcgtca gaccagatag ctttgatgtc |
| 301 | ctgatcggaa ggatcgttgg cccccctgca ggcagctgtt aattaactgc gcgctcgctc |
| 361 | gctcactgag gccgcccggg caaagcccgg gcgtcgggcg acctttggtc gcccggcctc |
| 421 | agtgagcgag cgagcgcgca gagagggagt ggccaactcc atcactaggg gttccttgta |
| 481 | gttaatgatt aacccgccat gctacttatc tacggcgcgc cacgcgttct aggagttaat |
| 541 | ttttaaaaag cagtcaaaag tccaagtgcc cttgcgagca tttactctct ctgtttgctc |
| 601 | tggttaataa tctcaggagc acaaacattc cttactagtt ctaggagtta atttttaaaa |
| 661 | agcagtcaaa agtccaagtg cccttgcgag catttactct ctctgtttgc tctggttaat |
| 721 | aatctcagga gcacaaacat tccttactag ttctagagcg gccgccagtg tgctggaatt |
| 781 | cggctttttt agggctggaa gctacctttg acatcatttc ctctgcgaat gcatgtataa |
| 841 | tttctacaga acctattaga aaggatcacc cagcctctgc ttttgtacaa ctttccctta |
| 901 | aaaaactgcc aatcccactg ctgtttggcc caatagtgag aactttttcc tgctgcctct |
| 961 | tggtgctttt gcctatggcc cctattctgc ctgctgaaga cactcttgcc agcatggact |
| 1021 | taaacccctc cagctctgac aatcctcttt ctcttttgtt ttacatgaag ggtctggcag |
| 1081 | ccaaagcaat cactcaaagt tcaaacctta tcattttttg ctttgttcct cttggccttg |
| 1141 | gttttgtaca tcagctttga aaataccatc ccagggttaa tgctggggtt aatttataac |
| 1201 | tgagagtgct ctagttctgc aatacaggac atgctataaa aatggaaaga tgttgctttc |
| 1261 | tgagagatca ggtggattct tgggcatttg cttaagcttg gtaccgccac catgggagtg |
| 1321 | agacatcctc cttgttctca tagactgctg gctgtgtgtg ctctggtgtc tctggctaca |
| 1381 | gctcagcagg gagcttctag acctggacct agagatgctc aggctcatcc tggaagacct |
| 1441 | agagctgtgc ctacacagtg tgatgtgcct cctaattcta gatttgattg tgctcctgat |
| 1501 | aaggctataa cacaggagca gtgtgaggct agaggatgtt gttatattcc tgctaagcag |
| 1561 | ggactgcagg gagctcagat gggacagcct tggtgttttt ttcctccttc ttatccttct |
| 1621 | tataagctgg agaatctgtc ttcttctgag atgggatata cagctacact gacaagaaca |
| 1681 | acacctacat tttttcctaa ggatattctg acactgagac tggatgtgat gatggagaca |
| 1741 | gagaatagac tgcattttac aattaaggat cctgctaata gaagatatga ggtgcctcta |
| 1801 | gagacaccta gagtgcattc tagagctcct tctcctctgt attctgtgga gttttctgag |
| 1861 | gagccttttg gagtgattgt gcatagacag ctggatggaa gagtgctgct gaatacaaca |
| 1921 | gtggctcctc tgttttttgc tgatcagttt ctgcagctgt ctacatctct gccttctcag |
| 1981 | tatattacag gactggctga gcatctgtct cctctgatgc tgtctacatc ttggacaaga |
| 2041 | attacactgt ggaatagaga tctggctcct acacctggag ctaatctgta tggatctcat |
| 2101 | cctttttatc tggctctgga ggatggagga tctgctcatg gagtgtttct gctgaattct |
| 2161 | aatgctatgg atgtagtgct gcagccttct cctgctctgt cttggagatc tacaggagga |
| 2221 | attctggatg tgtatatttt tctaggacct gagcctaagt ctgtggtgca gcagtatctg |
| 2281 | gatgtagtgg gatatccttt tatgcctcct tattggggac tgggatttca tctgtgtaga |
| 2341 | tggggatatt cttctacagc tattacaaga caggtggttg agaatatgac aagagctcat |
| 2401 | tttcctctgg atgtgcagtg gaatgatcta gattatatgg attctagaag agattttaca |
| 2461 | tttaataagg atggatttag agattttcct gctatggtgc aggagctgca tcagggagga |
| 2521 | agaagatata tgatgattgt ggatcctgct atttcttctt ctggacctgc tggatcttat |
| 2581 | agaccttatg atgagggact gagaagagga gtgtttatta caaatgagac aggacagcct |
| 2641 | ctgattggaa aggtgtggcc tggatctaca gcttttcctg attttacaaa tcctacagct |
| 2701 | ctggcttggt gggaggatat ggtggctgag tttcatgatc aggtgccttt tgatggaatg |
| 2761 | tggattgata tgaatgagcc ttctaatttt ataagaggat ctgaggatgg atgtcctaat |
| 2821 | aatgagctag agaatcctcc ttatgtgcct ggagtagtgg gaggaacact gcaggctgct |
| 2881 | acaatttgtg cttcttctca tcagtttctg tctacacatt ataatctgca taatctgtat |
| 2941 | ggactgacag aggctattgc ttctcataga gctctggtga aggctagagg aacaagacct |
| 3001 | tttgtgattt ctagatctac atttgctgga catggaagat atgctggaca ttggacagga |
| 3061 | gatgtatggt cttcttggga gcagctagct tcttctgtgc ctgagattct gcagtttaat |
| 3121 | ctgctaggag tgcctctggt gggagctgat gtgtgtggat ttctgggaaa tacatctgag |
| 3181 | gagctgtgtg tgagatggac acagctggga gctttttatc cttttatgag aaatcataat |
| 3241 | tctctgctgt ctctgcctca ggagccttat tctttttctg agcctgctca gcaggctatg |
| 3301 | agaaaggctc tgacactgag atatgctctg ctgcctcatc tgtatacact gtttcatcag |
| 3361 | gctcatgtgg ctggagagac agtagctaga cctctgtttc tggagtttcc taaggattct |
| 3421 | tctacatgga cagttgatca tcagctgctg tggggagagg ctctgctgat tacacctgtg |
| 3481 | ctgcaggctg gaaaggctga ggtgacagga tattttcctc tgggaacatg gtatgatctg |
| 3541 | cagacagtgc ctatagaggc tctgggatct ctgcctcctc ctcctgctgc tcctagagag |
| 3601 | cctgctattc attctgaggg acagtgggtg acactgcctg ctcctctgga tacaattaat |
| 3661 | gtgcatctga gagctggata tattattcct ctgcagggac ctggactgac aacaacagag |
| 3721 | tctagacagc agcctatggc tctggctgtg gctctgacaa agggaggaga ggctagagga |
| 3781 | gagctgtttt gggatgatgg agagtctctg gaggtgctgg agagaggagc ttatacacag |
| 3841 | gtgatttttc tggctagaaa taatacaatt gtgaatgagc tggtgagagt gacatctgag |
| 3901 | ggagctggac tgcagctgca gaaggtgaca gtgctgggag tggctacagc tcctcagcag |
| 3961 | gtgctgtcta atggagtgcc tgtgtctaat tttacatatt ctcctgatac aaaggtgctg |
| 4021 | gatatttgtg tgtctctgct gatgggagag cagtttctgg tgtcttggtg ttaatgattt |
| 4081 | aaatctcgag ccgggcggag tgtgttagtc tctccagagg gaggctggtt ccccagggaa |
| 4141 | gcagagcctg tgtgcgggca gcagctgtgt gcgggcctgg gggttgttaa gtgcaattat |
| 4201 | ttttaataaa aggggcattt ggaaaaaaaa aaaaaaggta gcagtcgaca gatgaattct |
| 4261 | gcagatctgt ggcttctagc tgcccgggtg gcatccctgt gacccctccc cagtgcctct |
| 4321 | cctggccctg gaagttgcca ctccagtgcc caccagcctt gtcctaataa aattaagttg |
| 4381 | catcattttg tctgactagg tgtccttcta taatattatg gggtggaggg gggtggtatg |
| 4441 | gagcaagggg caagttggga agacaacctg tagggcctgc ggggtctatt gggaaccaag |
| 4501 | ctggagtgca gtggcacaat cttggctcac tgcaatctcc gcctcctggg ttcaagcgat |
| 4561 | tctcctgcct cagcctcccg agttgttggg attccaggca tgcatgacca ggctcagcta |
| 4621 | atttttgttt ttttggtaga gacggggttt caccatattg gccaggctgg tctccaactc |
| 4681 | ctaatctcag gtgatctacc caccttggcc tcccaaattg ctgggattac aggcgtgaac |
| 4741 | cactgctccc ttccctgtcc ttctgatttt aaaaaaaaga gaaatgttct ggcacctgca |
| 4801 | cttgcactgg ggacagccta ttttgctagt ttgttttgtt tcgttttgtt ttgatggaga |
| 4861 | gcgtatgttg tttaaacgcg gccgcgtaga taagtagcat ggcgggttaa tcattaacta |
| 4921 | caaggaaccc ctagtgatgg agttggccac tccctctctg cgcgctcgct cgctcactga |
| 4981 | ggccgggcga ccaaaggtcg cccgacgccc gggctttgcc cgggcggcct cagtgagcga |
| 5041 | gcgagcgcgc agttaattaa ggcgccctag gccgaccctt agactctgta ctcagttcta |
| 5101 | taaacgagcc attggatacg agatccgtag attgataagg gacacggaat atccccggac |
| 5161 | gcaatagaca ccggtggaca gcttggtatc ctgagcacag tcgcgcgtcc gaatctagct |
| 5221 | ctactttaga ggccccggat tctgatggtc gtagaccgca gaaccgattg gggggatgag |
| 5281 | atctactagt tatcagcaca caattgccca ttatacgcgc gtataatgga ctattgtgtg |
| 5341 | ctgatatagg gataacaggg taattctaga gctagcatat ggatccatcg atttgatgcg |
| 5401 | gtattttctc cttacgcatc tgtgcggtat ttcacaccgc atacgtcaaa gcaaccatag |
| 5461 | tacgcgccct gtagcggcgc attaagcgcg gcgggtgtgg tggttacgcg cagcgtgacc |
| 5521 | gctacacttg ccagcgccct agcgcccgct cctttcgctt tcttcccttc ctttctcgcc |
| 5581 | acgttcgccg gctttccccg tcaagctcta aatcgggggc tccctttagg gttccgattt |
| 5641 | agtgctttac ggcacctcga ccccaaaaaa cttgatttgg gtgatggttc acgtagtggg |
| 5701 | ccatcgccct gatagacggt ttttcgccct ttgacgttgg agtccacgtt ctttaatagt |
| 5761 | ggactcttgt tccaaactgg aacaacactc aactctatct cgggctattc ttttgattta |
| 5821 | taagggattt tgccgatttc ggtctattgg ttaaaaaatg agctgattta acaaaaattt |
| 5881 | aacgcgaatt ttaacaaaat attaacgttt acaattttat ggtgcactct cagtacaatc |
| 5941 | tgctctgatg ccgcatagtt aagccagccc cgacacccgc caacacccgc tgacgcgccc |
| 6001 | tgacgggctt gtctgctccc ggcatccgct tacagacaag ctgtgaccgt ctccgggagc |
| 6061 | tgcatgtgtc agaggttttc accgtcatca ccgaaacgcg cgagacgaaa gggcctcgtg |
| 6121 | atacgcctat ttttataggt taatgtcatg ataataatgg tttcttagac gtcaggtggc |
| 6181 | acttttcggg gaaatgtgcg cggaacccct atttgtttat ttttctaaat acattcaaat |
| 6241 | atgtatccgc tcatgagaca ataaccctga taaatgcttc aataatattg aaaaaggaag |
| 6301 | agtatgagcc atattcaacg ggaaacgtcg aggccgcgat taaattccaa catggatgct |
| 6361 | gatttatatg ggtataaatg ggctcgcgat aatgtcgggc aatcaggtgc gacaatctat |
| 6421 | cgcttgtatg ggaagcccga tgcgccagag ttgtttctga aacatggcaa aggtagcgtt |
| 6481 | gccaatgatg ttacagatga gatggtcaga ctaaactggc tgacggaatt tatgcctctt |
| 6541 | ccgaccatca agcattttat ccgtactcct gatgatgcat ggttactcac cactgcgatc |
| 6601 | cccggaaaaa cagcattcca ggtattagaa gaatatcctg attcaggtga aaatattgtt |
| 6661 | gatgcgctgg cagtgttcct gcgccggttg cattcgattc ctgtttgtaa ttgtcctttt |
| 6721 | aacagcgatc gcgtatttcg tctcgctcag gcgcaatcac gaatgaataa cggtttggtt |
| 6781 | gatgcgagtg attttgatga cgagcgtaat ggctggcctg ttgaacaagt ctggaaagaa |
| 6841 | atgcataaac ttttgccatt ctcaccggat tcagtcgtca ctcatggtga tttctcactt |
| 6901 | gataacctta tttttgacga ggggaaatta ataggttgta ttgatgttgg acgagtcgga |
| 6961 | atcgcagacc gataccagga tcttgccatc ctatggaact gcctcggtga gttttctcct |
| 7021 | tcattacaga aacggctttt tcaaaaatat ggtattgata atcctgatat gaataaattg |
| 7081 | cagtttcatt tgatgctcga tgagtttttc taagcgtata atggtctaga gctagcatat |
| 7141 | ggatccatcg attccattat acgcctgtca gaccaagttt actcatatat actttagatt |
| 7201 | gatttaaaac ttcattttta atttaaaagg atctaggtga agatcctttt tgataatctc |
| 7261 | atgaccaaaa tcccttaacg tgagttttcg ttccactgag cgtcagaccc cgtagaaaag |
| 7321 | atcaaaggat cttcttgaga tccttttttt ctgcgcgtaa tctgctgctt gcaaacaaaa |
| 7381 | aaaccaccgc taccagcggt ggtttgtttg ccggatcaag agctaccaac tctttttccg |
| 7441 | aaggtaactg gcttcagcag agcgcagata ccaaatactg ttcttctagt gtagccgtag |
| 7501 | ttaggccacc acttcaagaa ctctgtagca ccgcctacat acctcgctct gctaatcctg |
| 7561 | ttaccagtgg ctgctgccag tggcgataag tcgtgtctta ccgggttgga ctcaagacga |
| 7621 | tagttaccgg ataaggcgca gcggtcgggc tgaacggggg gttcgtgcac acagcccagc |
| 7681 | ttggagcgaa cgacctacac cgaactgaga tacctacagc gtgagctatg agaaagcgcc |
| 7741 | acgcttcccg aagggagaaa ggcggacagg tatccggtaa gcggcagggt cggaacagga |
| 7801 | gagcgcacga gggagcttcc agggggaaac gcctggtatc tttatagtcc tgtcgggttt |
| 7861 | cgccacctct gacttgagcg tcgatttttg tgatgctcgt caggggggcg gagcctatgg |
| 7921 | aaaaacgcca gcaacgcggc ctttttacgg ttcctggcct tttgctggcc ttttgctcac |
| 7981 | atgt |

| pP110 (SEQ ID NO: 502) | |
|---|---|
| (SEQ ID NO: 502) | |
| 1 | ctgcgcgctc gctcgctcac tgaggccgcc cgggcaaagc ccgggcgtcg ggcgaccttt |
| 61 | ggtcgcccgg cctcagtgag cgagcgagcg cgcagagagg gagtggccaa ctccatcact |
| 121 | aggggttcct ggaggggtgg agtcgtgacg tgaattacgt catagggtta gggaggtcgg |
| 181 | ccgctctagg agttaatttt taaaaagcag tcaaaagtcc aagtgccctt gcgagcattt |
| 241 | actctctctg tttgctctgg ttaataatct caggagcaca aacattcctt actagttcta |
| 301 | ggagttaatt tttaaaaagc agtcaaaagt ccaagtgccc ttgcgagcat ttactctctc |
| 361 | tgtttgctct ggttaataat ctcaggagca caaacattcc ttactagttc tagagcggcc |
| 421 | gccagtgtgc tggaattcgg cttttttagg gctggaagct acctttgaca tcatttcctc |
| 481 | tgcgaatgca tgtataattt ctacagaacc tattagaaag gatcacccag cctctgcttt |
| 541 | tgtacaactt tcccttaaaa aactgccaat cccactgctg tttggcccaa tagtgagaac |
| 601 | tttttcctgc tgcctcttgg tgcttttgcc tatggcccct attctgcctg ctgaagacac |
| 661 | tcttgccagc atggacttaa acccctccag ctctgacaat cctctttctc ttttgtttta |
| 721 | catgaagggt ctggcagcca aagcaatcac tcaaagttca aaccttatca ttttttgctt |
| 781 | tgttcctctt ggccttggtt ttgtacatca gctttgaaaa taccatccca gggttaatgc |
| 841 | tggggttaat ttataactga gagtgctcta gttctgcaat acaggacatg ctataaaaat |
| 901 | ggaaagatgt tgctttctga gagatcagct tacatgtggt accgagctcg gatccactag |
| 961 | taacggccgc cagtgtgctg gaattcggct ttccaggcca tctccaacca tgggagtgag |
| 1021 | gcacccgccc tgctcccacc ggctcctggc cgtctgcgcc ctcgtgtcct tggcaaccgc |
| 1081 | tgcactcctg gggcagcagg gagccagcag accagggccc cgggatgccc aggcacaccc |
| 1141 | cggccgtccc agagcagtgc ccacacagtg cgacgtcccc cccaacagcc gcttcgattg |
| 1201 | cgcccctgac aaggccatca cccaggaaca gtgcgaggcc cgcggctgct gctacatccc |
| 1261 | tgcaaagcag gggctgcagg gagcccagat ggggcagccc tggtgcttct tcccacccag |
| 1321 | ctaccccagc tacaagctgg agaacctgag ctcctctgaa atgggctaca cggccaccct |
| 1381 | gacccgtacc acccccacct tcttccccaa ggacatcctg accctgcggc tggacgtgat |
| 1441 | gatggagact gagaaccgcc tccacttcac gatcaaagat ccagctaaca ggcgctacga |
| 1501 | ggtgcccttg gagaccccgc gtgtccacag ccgggcaccg tccccactct acagcgtgga |
| 1561 | gttctccgag gagcccttcg gggtgatcgt gcaccggcag ctggacggcc gcgtgctgct |
| 1621 | gaacacgacg gtggcgcccc tgttctttgc ggaccagttc cttcagctgt ccacctcgct |
| 1681 | gccctcgcag tatatcacag gcctcgccga gcacctcagt cccctgatgc tcagcaccag |
| 1741 | ctggaccagg atcaccctgt ggaaccggga ccttgcgccc acgcccggtg cgaacctcta |
| 1801 | cgggtctcac cctttctacc tggcgctgga ggacggcggg tcggcacacg gggtgttcct |
| 1861 | gctaaacagc aatgccatgg atgtggtcct gcagccgagc cctgccctta gctggaggtc |
| 1921 | gacaggtggg atcctggatg tctacatctt cctgggccca gagcccaaga gcgtggtgca |
| 1981 | gcagtacctg gacgttgtgg gatacccgtt catgccgcca tactggggcc tgggcttcca |
| 2041 | cctgtgccgc tggggctact cctccaccgc tatcacccgc caggtggtgg agaacatgac |
| 2101 | cagggcccac ttccccctgg acgtccaatg gaacgacctg gactacatgg actcccggag |
| 2161 | ggacttcacg ttcaacaagg atggcttccg ggacttcccg gccatggtgc aggagctgca |
| 2221 | ccagggcggc cggcgctaca tgatgatcgt ggatcctgcc atcagcagct cgggccctgc |
| 2281 | cgggagctac aggccctacg acgagggtct gcggaggggg gttttcatca ccaacgagac |
| 2341 | cggccagccg ctgattggga aggtatggcc cgggtccact gccttccccg acttcaccaa |
| 2401 | ccccacagcc ctggcctggt gggaggacat ggtggctgag ttccatgacc aggtgccctt |
| 2461 | cgacggcatg tggattgaca tgaacgagcc ttccaacttc atcagaggct ctgaggacgg |
| 2521 | ctgccccaac aatgagctgg agaacccacc ctacgtgcct ggggtggttg gggggaccct |
| 2581 | ccaggcggcc accatctgtg cctccagcca ccagtttctc tccacacact acaacctgca |
| 2641 | caacctctac ggcctgaccg aagccatcgc ctcccacagg gcgctggtga aggctcgggg |
| 2701 | gacacgccca tttgtgatct cccgctcgac ctttgctggc cacggccgat acgccggcca |
| 2761 | ctggacgggg gacgtgtgga gctcctggga gcagctcgcc tcctccgtgc cagaaatcct |
| 2821 | gcagtttaac ctgctggggg tgcctctggt cggggccgac gtctgcggct tcctgggcaa |
| 2881 | cacctcagag gagctgtgtg tgcgctggac ccagctgggg gccttctacc ccttcatgcg |
| 2941 | gaaccacaac agcctgctca gtctgcccca ggagccgtac agcttcagcg agccggccca |
| 3001 | gcaggccatg aggaaggccc tcaccctgcg ctacgcactc ctcccccacc tctacacact |
| 3061 | gttccaccag gcccacgtcg cgggggagac cgtggcccgg cccctcttcc tggagttccc |
| 3121 | caaggactct agcacctgga ctgtggacca ccagctcctg tggggggagg ccctgctcat |
| 3181 | caccccagtg ctccaggccg ggaaggccga agtgactggc tacttcccct tgggcacatg |
| 3241 | gtacgacctg cagacggtgc caatagaggc ccttggcagc ctcccacccc cacctgcagc |
| 3301 | tccccgtgag ccagccatcc acagcgaggg gcagtgggtg acgctgccgg cccccctgga |
| 3361 | caccatcaac gtccacctcc gggctgggta catcatcccc ctgcagggcc ctggcctcac |
| 3421 | aaccacagag tcccgccagc agcccatggc cctggctgtg gccctgacca agggtggaga |
| 3481 | ggcccgaggg gagctgttct gggacgatgg agagagcctg gaagtgctgg agcgaggggc |
| 3541 | ctacacacag gtcatcttcc tggccaggaa taacacgatc gtgaatgagc tggtacgtgt |
| 3601 | gaccagtgag ggagctggcc tgcagctgca gaaggtgact gtcctgggcg tggccacggc |
| 3661 | gccccagcag gtcctctcca acggtgtccc tgtctccaac ttcacctaca gccccgacac |
| 3721 | caaggtcctg gacatctgtg tctcgctgtt gatgggagag cagtttctcg tcagctggtg |
| 3781 | ttagccgggc ggagtgtgtt agtctctcca gagggaggct ggttccccag ggaagcagag |
| 3841 | cctgtgtgcg ggcagcagct gtgtgcgggc ctgggggttg ttaagtgcaa ttatttttaa |
| 3901 | taaaaggggc atttggaaaa aaaaaaaaaa ggtagcagtc gacagatgaa ttctgcagat |
| 3961 | ctgtggcttc tagctgcccg ggtggcatcc ctgtgacccc tccccagtgc ctctcctggc |
| 4021 | cctggaagtt gccactccag tgcccaccag ccttgtccta ataaaattaa gttgcatcat |
| 4081 | tttgtctgac taggtgtcct tctataatat tatggggtgg aggggggtgg tatggagcaa |
| 4141 | ggggcaagtt gggaagacaa cctgagttgt tgggattcca ggcatcgagt agataagtag |
| 4201 | catggcgggt taatcattaa ctacaaggaa cccctagtga tggagttggc cactccctct |
| 4261 | ctgcgcgctc gctcgctcac tgaggccggg cgaccaaagg tcgcccgacg cccgggcttt |
| 4321 | gcccgggcgg cctcagtgag cgagcgagcg cgcagagagg gacagatccg aagccgggca |
| 4381 | aatcagcgcc tggcagcagt ggcgtctggc ggaaaacctc agtgtgacgc tccccgccgc |
| 4441 | gtcccacgct tgttcccgga tctgaccacc agcgaaatcc gatttttgca ccgagctggg |
| 4501 | taataagcgt tggcaattta accgccagtc aggctttctt tcacagtgtg gattggcgat |
| 4561 | aaaaaacaac tgctgacgcc gctgcgcgat cagttcaccc gttcaccgct ggataacgac |
| 4621 | ttggcgtaag tgaagcgacc cgtaagaccc taacgcctgg gtcgaacgct ggaaggcggc |
| 4681 | gggccaaacc aggccgaagc agcgttgttg cagttcacgg cagatacact tgctgttgcg |
| 4741 | gtgctgatta cgaccgctca ctcgtggcag caacagggga aaaccttatt tatcagccgg |
| 4801 | aaaacctacc ggattgttgg tagtggtcaa taggcgatta ccgttgtgtt gaagtggcga |
| 4861 | gcgatacacc gcttccggcg cggattggcc tgaactgcca actggcgcag gtagcagagc |
| 4921 | gggtaaactg gctcggatta gggccgcaag aaaactatcc cgaccgcctt actgccgcct |
| 4981 | gttttgaccg ctgggatctg ccaagtcaga cagtatagcc cgtacgtctt cccgagcgaa |
| 5041 | aacggtctgc gctgcgggac gcgcgaattg aatttggccc acaccagtgg cgcggcgact |
| 5101 | tccagttcaa tatcagccgc tacagtgaac agcaactgtt ggaaaccagc cttcgccaac |
| 5161 | tgctgcacgc ggaagaaggc actggctgaa tatcgacggt ttccagttgg ggattggtgg |
| 5221 | cgacgactcc tggagcccgt cagtatcggc ggacttccaa ctgagcgccg gtcgctacct |
| 5281 | taccagttgg tctggtgtca aaaagcgtcc gcttgagtct agcgatcgcg cgcagatctg |
| 5341 | tcatgtgagc aaaaggccag caaaaggcca ggaaccgtaa aaaggccgcg ttgctggcgt |
| 5401 | ttttccatag gctccgcccc cctgacgagc atcacaaaaa tcgacgctca agtcagaggt |
| 5461 | ggcgaaaccc gacaggacta taaagatacc aggcgtttcc ccctggaagc tccctcgtgc |
| 5521 | gctctcctgt tccgaccctg ccgcttaccg gatacctgtc cgcctttctc ccttcgggaa |
| 5581 | gcgtggcgct ttctcatagc tcacgctgta ggtatctcag ttcggtgtag gtcgttcgct |
| 5641 | ccaagctggg ctgtgtgcac gaaccccccg ttcagcccga ccgctgcgcc ttatccggta |
| 5701 | actatcgtct tgagtccaac ccggtaagac acgacttatc gccactggca gcagccactg |
| 5761 | gtaacaggat tagcagagcg aggtatgtag gcggtgctac agagttcttg aagtggtggc |
| 5821 | ctaactacgg ctacactaga agaacagtat ttggtatctg cgctctgctg aagccagtta |
| 5881 | ccttcggaaa aagagttggt agctcttgat ccggcaaaca aaccaccgct ggtagcggtg |
| 5941 | gtttttttgt ttgcaagcag cagattacgc gcagaaaaaa aggatctcaa gaagatcctt |
| 6001 | tgatcttttc tacggggtct gacgctcagt ggaacgaaaa ctcacgttaa gggattttgg |
| 6061 | tcatgagatt atcaaaaagg atcttcacct agatcctttt cacgtagaaa gccagtccgc |
| 6121 | agaaacggtg ctgaccccgg atgaatgtca gctactgggc tatctggaca agggaaaacg |
| 6181 | caagcgcaaa gagaaagcag gtagcttgca gtgggcttac atggcgatag ctagactggg |
| 6241 | cggttttatg gacagcaagc gaaccggaat tgccagctgg ggcgccctct ggtaaggttg |
| 6301 | ggaagccctg caaagtaaac tggatggctt tcttgccgcc aaggatctga tggcgcaggg |
| 6361 | gatcaagatc tgatcaagag acaggatgag gatcgtttcg catgattgaa caagatggat |
| 6421 | tgcacgcagg ttctccggcc gcttgggtgg agaggctatt cggctatgac tgggcacaac |
| 6481 | agacaatcgg ctgctctgat gccgccgtgt tccggctgtc agcgcagggg cgcccggttc |
| 6541 | tttttgtcaa gaccgacctg tccggtgccc tgaatgaact gcaagacgag gcagcgcggc |
| 6601 | tatcgtggct ggccacgacg ggcgttcctt gcgcagctgt gctcgacgtt gtcactgaag |
| 6661 | cgggaaggga ctggctgcta ttgggcgaag tgccggggca ggatctcctg tcatctcacc |
| 6721 | ttgctcctgc cgagaaagta tccatcatgg ctgatgcaat gcggcggctg catacgcttg |
| 6781 | atccggctac ctgcccattc gaccaccaag cgaaacatcg catcgagcga gcacgtactc |
| 6841 | ggatggaagc cggtcttgtc gatcaggatg atctggacga agagcatcag gggctcgcgc |
| 6901 | cagccgaact gttcgccagg ctcaaggcga gcatgcccga cggcgaggat ctcgtcgtga |
| 6961 | cccatggcga tgcctgcttg ccgaatatca tggtggaaaa tggccgcttt tctggattca |
| 7021 | tcgactgtgg ccggctgggt gtggcggacc gctatcagga catagcgttg gctacccgtg |
| 7081 | atattgctga agagcttggc ggcgaatggg ctgaccgctt cctcgtgctt tacggtatcg |
| 7141 | ccgctcccga ttcgcagcgc atcgccttct atcgccttct tgacgagttc ttctgaattt |
| 7201 | aaagcccaat acgcaaaccg cctctccccg cgcgttggcc gaagcggttc aatattttgt |
| 7261 | taaaattcgc gttaaatttt tgttaaatca gctatttttt aaccaatagg ccgaaatcgg |
| 7321 | caaaatccct tgtaaatcaa aagaatagac cgagataggg ttgagtgttg ttccagtttg |
| 7381 | gaacaagagt ccactattaa agaacgtgga ctccaacgtc aaagggcgaa aaaccgtcta |
| 7441 | tcagggcgtt ggcccactac gtgaaccttc accctaatca agttttttgg ggtcgaggtg |
| 7501 | ccgtaaagca ctaaatcgga accctaaagg gagcccccga tttagagctt gacggggaaa |
| 7561 | ccggcgaacg tggcgagaaa ggaagggaag aaagcgaaag gagcgggcgc tagggcgctg |
| 7621 | gcaagtgtag cggtcacgct gcgcgtaacc accacacccg ccgcgctaag cgccgctaca |
| 7681 | gggcgcgtcc cttcgccttc aggctgcgtc gagtactgta ctgtgagcca gagttgcccg |
| 7741 | gcgctctccg gctgcggtag ttcaggcagt tcaatcaact gtttaccttg tggagcgact |
| 7801 | ccagaggcac ttcaccgctt gccagcggct tacgatccag cgccacgatc cagtgcagga |
| 7861 | gatcgttatc gctatacgga acaggtattc gctggtcact tcgataaggt ttgcccggat |
| 7921 | aaacggaact ggaaaaactg ctgctggtgt tttgcttccg tcagtgctgg atcggcgtgc |
| 7981 | ggtcggcaaa gaccagaccg ttctaacaga actggcgatt gttcggcgta tcgccaaaat |
| 8041 | caccgccgta agccgaccac gggttgccgt tttcagcagg atttaatcag cgactgatcc |
| 8101 | acccagtccc agacgaagcc gccctgtaaa cggggatact gacgaaacgc ctgccagtat |
| 8161 | ttagcgaaac cgccaagact gttacccaag cgtgggcgta ttcgcaaagg atcagcgggc |
| 8221 | gcgtctctcc aggtagcgaa agcctttttt gatcgacctt tcggcacagc cgggaagggc |
| 8281 | tggtcttcaa ccacgcgcgc gtacaacggg caaataatat cggtggccgt ggtgtcggct |
| 8341 | ccgccgcctt caactgcacc gggcgggaag gatcgacaga tttgatccag cgatacagcg |
| 8401 | cgtcgtgatt agcgccgtgg cctgattcaa ttccccagcg accagtagat cacactcggg |
| 8461 | tgattacgat tgcgctgcac cagtcgcgtt acggttcgct cttcgccggt agccagcgcg |
| 8521 | gatcacggtc agacgattcg ttggcacgat ccgtgggttt caatactggc ttcaaaccac |
| 8581 | cactaacagg ccgtagcggt cgcacagcgt gtaccacagc ggttggttcg gataatcgaa |
| 8641 | cagcgcacgg cgttaaagtt gttctgcttc aacagcagga tattctgcac cttcgtctgc |
| 8701 | tcttcctaac ctgaccaagc agaggatctg ctcgtgacgg ttaatcctcg aatcagcaac |
| 8761 | ggcttgccgt tcagcagcag cagaccaagt tcaatccgca cctcgcggaa accgacaacg |
| 8821 | caggcttctg cttcaatcag cgtgccgtcg gcggtgtgca gttcaaccac cgcacgatag |
| 8881 | agattcggga tttcggcgct ccacagtttc gggttttcga cgttcagacg tagtgtgacg |
| 8941 | cgatctgcaa accaccacgc tcaacgataa tttcaccgcc gaaaggcgcg gtgccgctgg |
| 9001 | cgacctgcgt ttcaccctgc cagaaagaaa ctgttacccg taggtagtca cgcaactcgc |
| 9061 | cgcacactga acttcagcct ccagtacagc gcggctgaaa tcgtcttaaa gcgagtggca |
| 9121 | actggaaatc gctgatttgt gtagtcggtt tagcagcaac gagacttcac ggaaaatccg |
| 9181 | ctaatccgcc acagatcctg atcttccaga taactgccgt cactccaacg cagcaccttc |
| 9241 | accgcgaggc ggttttctcc ggcgcgtaaa aatcgctcag gtcaaattca gacggcaaac |
| 9301 | gactgtcctg gccgtaaccg acccagcgcc cgttgcacca cagattgaaa cgccgagttt |
| 9361 | acgcctcaaa aataattcgc gtctggcctt cctgtagcca gctttcacaa ctataatagt |
| 9421 | gagcgagtaa caacccgtcg gattctccgt gggaacaaac ggcggattga ccgtataggg |
| 9481 | ataggttacg ttggtgtagt agggcgctcc gtaaccgtgc tactgccagt ttgaggggac |
| 9541 | gacgacagta tcggcctcag gaagatcgca ctccagccag ctttccggca ccgcttctgg |
| 9601 | tactggaaac caggcaaagc gcctatcgcc tatcaggctg cacaactgtt gggaagggcg |
| 9661 | atctgtgcgg gcctcttcgc tattacgcca gcttgcgaaa gggggtagtg ctgcaaggcg |
| 9721 | attaagttgg gtaacgccag ggttttccca gtcacgacgt ggatctgggc cactccctct |

| pP111 (SEQ ID NO: 503) | |
|---|---|
| (SEQ ID NO: 503) | |
| 1 | ctgcgcgctc gctcgctcac tgaggccgcc cgggcaaagc ccgggcgtcg ggcgaccttt |
| 61 | ggtcgcccgg cctcagtgag cgagcgagcg cgcagagagg gagtggccaa ctccatcact |
| 121 | aggggttcct ggaggggtgg agtcgtgacg tgaattacgt catagggtta gggaggtcgg |
| 181 | ccgctctagg agttaatttt taaaaagcag tcaaaagtcc aagtgccctt gcgagcattt |
| 241 | actctctctg tttgctctgg ttaataatct caggagcaca aacattcctt actagttcta |
| 301 | ggagttaatt tttaaaaagc agtcaaaagt ccaagtgccc ttgcgagcat ttactctctc |
| 361 | tgtttgctct ggttaataat ctcaggagca caaacattcc ttactagttc tagagcggcc |
| 421 | gccagtgtgc tggaattcgg cttttttagg gctggaagct acctttgaca tcatttcctc |
| 481 | tgcgaatgca tgtataattt ctacagaacc tattagaaag gatcacccag cctctgcttt |
| 541 | tgtacaactt tcccttaaaa aactgccaat cccactgctg tttggcccaa tagtgagaac |
| 601 | tttttcctgc tgcctcttgg tgcttttgcc tatggcccct attctgcctg ctgaagacac |
| 661 | tcttgccagc atggacttaa acccctccag ctctgacaat cctctttctc ttttgtttta |
| 721 | catgaagggt ctggcagcca aagcaatcac tcaaagttca aaccttatca ttttttgctt |
| 781 | tgttcctctt ggccttggtt ttgtacatca gctttgaaaa taccatccca gggttaatgc |
| 841 | tggggttaat ttataactga gagtgctcta gttctgcaat acaggacatg ctataaaaat |
| 901 | ggaaagatgt tgctttctga gagatcagct tacatgtggt accgagctcg gatccactag |
| 961 | taacggccgc cagtgtgctg gaattcggct ttccaggcca tctccaacca tgggagtgag |
| 1021 | gcacccgccc tgctcccacc ggctcctggc cgtctgcgcc ctcgtgtcct tggcaaccgc |
| 1081 | tcagcaggga gccagcagac cagggccccg ggatgcccag gcacaccccg gccgtcccag |
| 1141 | agcagtgccc acacagtgcg acgtcccccc caacagccgc ttcgattgcg cccctgacaa |
| 1201 | ggccatcacc caggaacagt gcgaggcccg cggctgctgc tacatccctg caaagcaggg |
| 1261 | gctgcaggga gcccagatgg ggcagccctg gtgcttcttc ccacccagct accccagcta |
| 1321 | caagctggag aacctgagct cctctgaaat gggctacacg gccaccctga cccgtaccac |
| 1381 | ccccaccttc ttccccaagg acatcctgac cctgcggctg gacgtgatga tggagactga |
| 1441 | gaaccgcctc cacttcacga tcaaagatcc agctaacagg cgctacgagg tgcccttgga |
| 1501 | gaccccgcgt gtccacagcc gggcaccgtc cccactctac agcgtggagt tctccgagga |
| 1561 | gcccttcggg gtgatcgtgc accggcagct ggacggccgc gtgctgctga acacgacggt |
| 1621 | ggcgcccctg ttctttgcgg accagttcct tcagctgtcc acctcgctgc cctcgcagta |
| 1681 | tatcacaggc ctcgccgagc acctcagtcc cctgatgctc agcaccagct ggaccaggat |
| 1741 | caccctgtgg aaccgggacc ttgcgcccac gcccggtgcg aacctctacg ggtctcaccc |
| 1801 | tttctacctg gcgctggagg acggcgggtc ggcacacggg gtgttcctgc taaacagcaa |
| 1861 | tgccatggat gtggtcctgc agccgagccc tgcccttagc tggaggtcga caggtgggat |
| 1921 | cctggatgtc tacatcttcc tgggcccaga gcccaagagc gtggtgcagc agtacctgga |
| 1981 | cgttgtggga tacccgttca tgccgccata ctggggcctg ggcttccacc tgtgccgctg |
| 2041 | gggctactcc tccaccgcta tcacccgcca ggtggtggag aacatgacca gggcccactt |
| 2101 | ccccctggac gtccaatgga acgacctgga ctacatggac tcccggaggg acttcacgtt |
| 2161 | caacaaggat ggcttccggg acttcccggc catggtgcag gagctgcacc agggcggccg |
| 2221 | gcgctacatg atgatcgtgg atcctgccat cagcagctcg ggccctgccg ggagctacag |
| 2281 | gccctacgac gagggtctgc ggaggggggt tttcatcacc aacgagaccg gccagccgct |
| 2341 | gattgggaag gtatggcccg ggtccactgc cttccccgac ttcaccaacc ccacagccct |
| 2401 | ggcctggtgg gaggacatgg tggctgagtt ccatgaccag gtgcccttcg acggcatgtg |
| 2461 | gattgacatg aacgagcctt ccaacttcat cagaggctct gaggacggct gccccaacaa |
| 2521 | tgagctggag aacccaccct acgtgcctgg ggtggttggg gggaccctcc aggcggccac |
| 2581 | catctgtgcc tccagccacc agtttctctc cacacactac aacctgcaca acctctacgg |
| 2641 | cctgaccgaa gccatcgcct cccacagggc gctggtgaag gctcggggga cacgcccatt |
| 2701 | tgtgatctcc cgctcgacct ttgctggcca cggccgatac gccggccact ggacggggga |
| 2761 | cgtgtggagc tcctgggagc agctcgcctc ctccgtgcca gaaatcctgc agtttaacct |
| 2821 | gctgggggtg cctctggtcg gggccgacgt ctgcggcttc ctgggcaaca cctcagagga |
| 2881 | gctgtgtgtg cgctggaccc agctgggggc cttctacccc ttcatgcgga accacaacag |
| 2941 | cctgctcagt ctgccccagg agccgtacag cttcagcgag ccggcccagc aggccatgag |
| 3001 | gaaggccctc accctgcgct acgcactcct cccccacctc tacacactgt tccaccaggc |
| 3061 | ccacgtcgcg ggggagaccg tggcccggcc cctcttcctg gagttcccca aggactctag |
| 3121 | cacctggact gtggaccacc agctcctgtg gggggaggcc ctgctcatca ccccagtgct |
| 3181 | ccaggccggg aaggccgaag tgactggcta cttccccttg ggcacatggt acgacctgca |
| 3241 | gacggtgcca atagaggccc ttggcagcct cccaccccca cctgcagctc cccgtgagcc |
| 3301 | agccatccac agcgaggggc agtgggtgac gctgccggcc cccctggaca ccatcaacgt |
| 3361 | ccacctccgg gctgggtaca tcatccccct gcagggccct ggcctcacaa ccacagagtc |
| 3421 | ccgccagcag cccatggccc tggctgtggc cctgaccaag ggtggagagg cccgagggga |
| 3481 | gctgttctgg gacgatggag agagcctgga agtgctggag cgaggggcct acacacaggt |
| 3541 | catcttcctg gccaggaata acacgatcgt gaatgagctg gtacgtgtga ccagtgaggg |
| 3601 | agctggcctg cagctgcaga aggtgactgt cctgggcgtg gccacggcgc cccagcaggt |
| 3661 | cctctccaac ggtgtccctg tctccaactt cacctacagc cccgacacca aggtcctgga |
| 3721 | catctgtgtc tcgctgttga tgggagagca gtttctcgtc agctggtgtt agccgggcgg |
| 3781 | agtgtgttag tctctccaga gggaggctgg ttccccaggg aagcagagcc tgtgtgcggg |
| 3841 | cagcagctgt gtgcgggcct gggggttgtt aagtgcaatt atttttaata aaaggggcat |
| 3901 | ttggaaaaaa aaaaaaaagg tagcagtcga cagatgaatt ctgcagatct gtggcttcta |
| 3961 | gctgcccggg tggcatccct gtgacccctc cccagtgcct ctcctggccc tggaagttgc |
| 4021 | cactccagtg cccaccagcc ttgtcctaat aaaattaagt tgcatcattt tgtctgacta |
| 4081 | ggtgtccttc tataatatta tggggtggag gggggtggta tggagcaagg ggcaagttgg |
| 4141 | gaagacaacc tgagttgttg ggattccagg catcgagtag ataagtagca tggcgggtta |
| 4201 | atcattaact acaaggaacc cctagtgatg gagttggcca ctccctctct gcgcgctcgc |
| 4261 | tcgctcactg aggccgggcg accaaaggtc gcccgacgcc cgggctttgc ccgggcggcc |
| 4321 | tcagtgagcg agcgagcgcg cagagaggga cagatccgaa gccgggcaaa tcagcgcctg |
| 4381 | gcagcagtgg cgtctggcgg aaaacctcag tgtgacgctc cccgccgcgt cccacgcttg |
| 4441 | ttcccggatc tgaccaccag cgaaatccga tttttgcacc gagctgggta ataagcgttg |
| 4501 | gcaatttaac cgccagtcag gctttctttc acagtgtgga ttggcgataa aaaacaactg |
| 4561 | ctgacgccgc tgcgcgatca gttcacccgt tcaccgctgg ataacgactt ggcgtaagtg |
| 4621 | aagcgacccg taagacccta acgcctgggt cgaacgctgg aaggcggcgg gccaaaccag |
| 4681 | gccgaagcag cgttgttgca gttcacggca gatacacttg ctgttgcggt gctgattacg |
| 4741 | accgctcact cgtggcagca acaggggaaa accttattta tcagccggaa aacctaccgg |
| 4801 | attgttggta gtggtcaata ggcgattacc gttgtgttga agtggcgagc gatacaccgc |
| 4861 | ttccggcgcg gattggcctg aactgccaac tggcgcaggt agcagagcgg gtaaactggc |
| 4921 | tcggattagg gccgcaagaa aactatcccg accgccttac tgccgcctgt tttgaccgct |
| 4981 | gggatctgcc aagtcagaca gtatagcccg tacgtcttcc cgagcgaaaa cggtctgcgc |
| 5041 | tgcgggacgc gcgaattgaa tttggcccac accagtggcg cggcgacttc cagttcaata |
| 5101 | tcagccgcta cagtgaacag caactgttgg aaaccagcct tcgccaactg ctgcacgcgg |
| 5161 | aagaaggcac tggctgaata tcgacggttt ccagttgggg attggtggcg acgactcctg |
| 5221 | gagcccgtca gtatcggcgg acttccaact gagcgccggt cgctacctta ccagttggtc |
| 5281 | tggtgtcaaa aagcgtccgc ttgagtctag cgatcgcgcg cagatctgtc atgtgagcaa |
| 5341 | aaggccagca aaaggccagg aaccgtaaaa aggccgcgtt gctggcgttt ttccataggc |
| 5401 | tccgcccccc tgacgagcat cacaaaaatc gacgctcaag tcagaggtgg cgaaacccga |
| 5461 | caggactata aagataccag gcgtttcccc ctggaagctc cctcgtgcgc tctcctgttc |
| 5521 | cgaccctgcc gcttaccgga tacctgtccg cctttctccc ttcgggaagc gtggcgcttt |
| 5581 | ctcatagctc acgctgtagg tatctcagtt cggtgtaggt cgttcgctcc aagctgggct |
| 5641 | gtgtgcacga accccccgtt cagcccgacc gctgcgcctt atccggtaac tatcgtcttg |
| 5701 | agtccaaccc ggtaagacac gacttatcgc cactggcagc agccactggt aacaggatta |
| 5761 | gcagagcgag gtatgtaggc ggtgctacag agttcttgaa gtggtggcct aactacggct |
| 5821 | acactagaag aacagtattt ggtatctgcg ctctgctgaa gccagttacc ttcggaaaaa |
| 5881 | gagttggtag ctcttgatcc ggcaaacaaa ccaccgctgg tagcggtggt ttttttgttt |
| 5941 | gcaagcagca gattacgcgc agaaaaaaag gatctcaaga agatcctttg atcttttcta |
| 6001 | cggggtctga cgctcagtgg aacgaaaact cacgttaagg gattttggtc atgagattat |
| 6061 | caaaaaggat cttcacctag atccttttca cgtagaaagc cagtccgcag aaacggtgct |
| 6121 | gaccccggat gaatgtcagc tactgggcta tctggacaag ggaaaacgca agcgcaaaga |
| 6181 | gaaagcaggt agcttgcagt gggcttacat ggcgatagct agactgggcg gttttatgga |
| 6241 | cagcaagcga accggaattg ccagctgggg cgccctctgg taaggttggg aagccctgca |
| 6301 | aagtaaactg gatggctttc ttgccgccaa ggatctgatg gcgcagggga tcaagatctg |
| 6361 | atcaagagac aggatgagga tcgtttcgca tgattgaaca agatggattg cacgcaggtt |
| 6421 | ctccggccgc ttgggtggag aggctattcg gctatgactg ggcacaacag acaatcggct |
| 6481 | gctctgatgc cgccgtgttc cggctgtcag cgcaggggcg cccggttctt tttgtcaaga |
| 6541 | ccgacctgtc cggtgccctg aatgaactgc aagacgaggc agcgcggcta tcgtggctgg |
| 6601 | ccacgacggg cgttccttgc gcagctgtgc tcgacgttgt cactgaagcg ggaagggact |
| 6661 | ggctgctatt gggcgaagtg ccggggcagg atctcctgtc atctcacctt gctcctgccg |
| 6721 | agaaagtatc catcatggct gatgcaatgc ggcggctgca tacgcttgat ccggctacct |
| 6781 | gcccattcga ccaccaagcg aaacatcgca tcgagcgagc acgtactcgg atggaagccg |
| 6841 | gtcttgtcga tcaggatgat ctggacgaag agcatcaggg gctcgcgcca gccgaactgt |
| 6901 | tcgccaggct caaggcgagc atgcccgacg gcgaggatct cgtcgtgacc catggcgatg |
| 6961 | cctgcttgcc gaatatcatg gtggaaaatg gccgcttttc tggattcatc gactgtggcc |
| 7021 | ggctgggtgt ggcggaccgc tatcaggaca tagcgttggc tacccgtgat attgctgaag |
| 7081 | agcttggcgg cgaatgggct gaccgcttcc tcgtgcttta cggtatcgcc gctcccgatt |
| 7141 | cgcagcgcat cgccttctat cgccttcttg acgagttctt ctgaatttaa agcccaatac |
| 7201 | gcaaaccgcc tctccccgcg cgttggccga agcggttcaa tattttgtta aaattcgcgt |
| 7261 | taaatttttg ttaaatcagc tattttttaa ccaataggcc gaaatcggca aaatcccttg |
| 7321 | taaatcaaaa gaatagaccg agatagggtt gagtgttgtt ccagtttgga acaagagtcc |
| 7381 | actattaaag aacgtggact ccaacgtcaa agggcgaaaa accgtctatc agggcgttgg |
| 7441 | cccactacgt gaaccttcac cctaatcaag ttttttgggg tcgaggtgcc gtaaagcact |
| 7501 | aaatcggaac cctaaaggga gcccccgatt tagagcttga cggggaaacc ggcgaacgtg |
| 7561 | gcgagaaagg aagggaagaa agcgaaagga gcgggcgcta gggcgctggc aagtgtagcg |
| 7621 | gtcacgctgc gcgtaaccac cacacccgcc gcgctaagcg ccgctacagg gcgcgtccct |
| 7681 | tcgccttcag gctgcgtcga gtactgtact gtgagccaga gttgcccggc gctctccggc |
| 7741 | tgcggtagtt caggcagttc aatcaactgt ttaccttgtg gagcgactcc agaggcactt |
| 7801 | caccgcttgc cagcggctta cgatccagcg ccacgatcca gtgcaggaga tcgttatcgc |
| 7861 | tatacggaac aggtattcgc tggtcacttc gataaggttt gcccggataa acggaactgg |
| 7921 | aaaaactgct gctggtgttt tgcttccgtc agtgctggat cggcgtgcgg tcggcaaaga |
| 7981 | ccagaccgtt ctaacagaac tggcgattgt tcggcgtatc gccaaaatca ccgccgtaag |
| 8041 | ccgaccacgg gttgccgttt tcagcaggat ttaatcagcg actgatccac ccagtcccag |
| 8101 | acgaagccgc cctgtaaacg gggatactga cgaaacgcct gccagtattt agcgaaaccg |
| 8161 | ccaagactgt tacccaagcg tgggcgtatt cgcaaaggat cagcgggcgc gtctctccag |
| 8221 | gtagcgaaag ccttttttga tcgacctttc ggcacagccg ggaagggctg gtcttcaacc |
| 8281 | acgcgcgcgt acaacgggca aataatatcg gtggccgtgg tgtcggctcc gccgccttca |
| 8341 | actgcaccgg gcgggaagga tcgacagatt tgatccagcg atacagcgcg tcgtgattag |
| 8401 | cgccgtggcc tgattcaatt ccccagcgac cagtagatca cactcgggtg attacgattg |
| 8461 | cgctgcacca gtcgcgttac ggttcgctct tcgccggtag ccagcgcgga tcacggtcag |
| 8521 | acgattcgtt ggcacgatcc gtgggtttca atactggctt caaaccacca ctaacaggcc |
| 8581 | gtagcggtcg cacagcgtgt accacagcgg ttggttcgga taatcgaaca gcgcacggcg |
| 8641 | ttaaagttgt tctgcttcaa cagcaggata ttctgcacct tcgtctgctc ttcctaacct |
| 8701 | gaccaagcag aggatctgct cgtgacggtt aatcctcgaa tcagcaacgg cttgccgttc |
| 8761 | agcagcagca gaccaagttc aatccgcacc tcgcggaaac cgacaacgca ggcttctgct |
| 8821 | tcaatcagcg tgccgtcggc ggtgtgcagt tcaaccaccg cacgatagag attcgggatt |
| 8881 | tcggcgctcc acagtttcgg gttttcgacg ttcagacgta gtgtgacgcg atctgcaaac |
| 8941 | caccacgctc aacgataatt tcaccgccga aaggcgcggt gccgctggcg acctgcgttt |
| 9001 | caccctgcca gaaagaaact gttacccgta ggtagtcacg caactcgccg cacactgaac |
| 9061 | ttcagcctcc agtacagcgc ggctgaaatc gtcttaaagc gagtggcaac tggaaatcgc |
| 9121 | tgatttgtgt agtcggttta gcagcaacga gacttcacgg aaaatccgct aatccgccac |
| 9181 | agatcctgat cttccagata actgccgtca ctccaacgca gcaccttcac cgcgaggcgg |
| 9241 | ttttctccgg cgcgtaaaaa tcgctcaggt caaattcaga cggcaaacga ctgtcctggc |
| 9301 | cgtaaccgac ccagcgcccg ttgcaccaca gattgaaacg ccgagtttac gcctcaaaaa |
| 9361 | taattcgcgt ctggccttcc tgtagccagc tttcacaact ataatagtga gcgagtaaca |
| 9421 | acccgtcgga ttctccgtgg gaacaaacgg cggattgacc gtatagggat aggttacgtt |
| 9481 | ggtgtagtag ggcgctccgt aaccgtgcta ctgccagttt gaggggacga cgacagtatc |
| 9541 | ggcctcagga agatcgcact ccagccagct ttccggcacc gcttctggta ctggaaacca |
| 9601 | ggcaaagcgc ctatcgccta tcaggctgca caactgttgg gaagggcgat ctgtgcgggc |
| 9661 | ctcttcgcta ttacgccagc ttgcgaaagg gggtagtgct gcaaggcgat taagttgggt |
| 9721 | aacgccaggg ttttcccagt cacgacgtgg atctgggcca ctccctct |

| pP112 (SEQ ID NO: 504) | |
|---|---|
| (SEQ ID NO: 504) | |
| 1 | ctgcgcgctc gctcgctcac tgaggccgcc cgggcaaagc ccgggcgtcg ggcgaccttt |
| 61 | ggtcgcccgg cctcagtgag cgagcgagcg cgcagagagg gagtggccaa ctccatcact |
| 121 | aggggttcct ggaggggtgg agtcgtgacg tgaattacgt catagggtta gggaggtcgg |
| 181 | ccgctctagg agttaatttt taaaaagcag tcaaaagtcc aagtgccctt gcgagcattt |
| 241 | actctctctg tttgctctgg ttaataatct caggagcaca aacattcctt actagttcta |
| 301 | ggagttaatt tttaaaaagc agtcaaaagt ccaagtgccc ttgcgagcat ttactctctc |
| 361 | tgtttgctct ggttaataat ctcaggagca caaacattcc ttactagttc tagagcggcc |
| 421 | gccagtgtgc tggaattcgg cttttttagg gctggaagct acctttgaca tcatttcctc |
| 481 | tgcgaatgca tgtataattt ctacagaacc tattagaaag gatcacccag cctctgcttt |
| 541 | tgtacaactt tcccttaaaa aactgccaat cccactgctg tttggcccaa tagtgagaac |
| 601 | tttttcctgc tgcctcttgg tgcttttgcc tatggcccct attctgcctg ctgaagacac |
| 661 | tcttgccagc atggacttaa acccctccag ctctgacaat cctctttctc ttttgtttta |
| 721 | catgaagggt ctggcagcca aagcaatcac tcaaagttca aaccttatca ttttttgctt |
| 781 | tgttcctctt ggccttggtt ttgtacatca gctttgaaaa taccatccca gggttaatgc |
| 841 | tggggttaat ttataactga gagtgctcta gttctgcaat acaggacatg ctataaaaat |
| 901 | ggaaagatgt tgctttctga gagatcagct tacatgtggt accgagctcg gatccactag |
| 961 | taacggccgc cagtgtgctg gaattcggct ttccaggcca tctccaacca tgggagtgag |
| 1021 | gcacccgccc tgctcccacc ggctcctggc cgtctgcgcc ctcgtgtcct tggcaaccgc |
| 1081 | tgcactcctg gagtttgggc tgagctgggt ctttctggtg gccctgctga agggagtcca |
| 1141 | gtgtgagcag cagggagcca gcagaccagg gccccgggat gcccaggcac accccggccg |
| 1201 | tcccagagca gtgcccacac agtgcgacgt cccccccaac agccgcttcg attgcgcccc |
| 1261 | tgacaaggcc atcacccagg aacagtgcga ggcccgcggc tgctgctaca tccctgcaaa |
| 1321 | gcaggggctg cagggagccc agatggggca gccctggtgc ttcttcccac ccagctaccc |
| 1381 | cagctacaag ctggagaacc tgagctcctc tgaaatgggc tacacggcca ccctgacccg |
| 1441 | taccaccccc accttcttcc ccaaggacat cctgaccctg cggctggacg tgatgatgga |
| 1501 | gactgagaac cgcctccact tcacgatcaa agatccagct aacaggcgct acgaggtgcc |
| 1561 | cttggagacc ccgcgtgtcc acagccgggc accgtcccca ctctacagcg tggagttctc |
| 1621 | cgaggagccc ttcggggtga tcgtgcaccg gcagctggac ggccgcgtgc tgctgaacac |
| 1681 | gacggtggcg cccctgttct ttgcggacca gttccttcag ctgtccacct cgctgccctc |
| 1741 | gcagtatatc acaggcctcg ccgagcacct cagtcccctg atgctcagca ccagctggac |
| 1801 | caggatcacc ctgtggaacc gggaccttgc gcccacgccc ggtgcgaacc tctacgggtc |
| 1861 | tcaccctttc tacctggcgc tggaggacgg cgggtcggca cacggggtgt tcctgctaaa |
| 1921 | cagcaatgcc atggatgtgg tcctgcagcc gagccctgcc cttagctgga ggtcgacagg |
| 1981 | tgggatcctg gatgtctaca tcttcctggg cccagagccc aagagcgtgg tgcagcagta |
| 2041 | cctggacgtt gtgggatacc cgttcatgcc gccatactgg ggcctgggct tccacctgtg |
| 2101 | ccgctggggc tactcctcca ccgctatcac ccgccaggtg gtggagaaca tgaccagggc |
| 2161 | ccacttcccc ctggacgtcc aatggaacga cctggactac atggactccc ggagggactt |
| 2221 | cacgttcaac aaggatggct tccgggactt cccggccatg gtgcaggagc tgcaccaggg |
| 2281 | cggccggcgc tacatgatga tcgtggatcc tgccatcagc agctcgggcc ctgccgggag |
| 2341 | ctacaggccc tacgacgagg gtctgcggag gggggttttc atcaccaacg agaccggcca |
| 2401 | gccgctgatt gggaaggtat ggcccgggtc cactgccttc cccgacttca ccaaccccac |
| 2461 | agccctggcc tggtgggagg acatggtggc tgagttccat gaccaggtgc ccttcgacgg |
| 2521 | catgtggatt gacatgaacg agccttccaa cttcatcaga ggctctgagg acggctgccc |
| 2581 | caacaatgag ctggagaacc caccctacgt gcctggggtg gttgggggga ccctccaggc |
| 2641 | ggccaccatc tgtgcctcca gccaccagtt tctctccaca cactacaacc tgcacaacct |
| 2701 | ctacggcctg accgaagcca tcgcctccca cagggcgctg gtgaaggctc gggggacacg |
| 2761 | cccatttgtg atctcccgct cgacctttgc tggccacggc cgatacgccg gccactggac |
| 2821 | gggggacgtg tggagctcct gggagcagct cgcctcctcc gtgccagaaa tcctgcagtt |
| 2881 | taacctgctg ggggtgcctc tggtcggggc cgacgtctgc ggcttcctgg gcaacacctc |
| 2941 | agaggagctg tgtgtgcgct ggacccagct gggggccttc taccccttca tgcggaacca |
| 3001 | caacagcctg ctcagtctgc cccaggagcc gtacagcttc agcgagccgg cccagcaggc |
| 3061 | catgaggaag gccctcaccc tgcgctacgc actcctcccc cacctctaca cactgttcca |
| 3121 | ccaggcccac gtcgcggggg agaccgtggc ccggcccctc ttcctggagt tccccaagga |
| 3181 | ctctagcacc tggactgtgg accaccagct cctgtggggg gaggccctgc tcatcacccc |
| 3241 | agtgctccag gccgggaagg ccgaagtgac tggctacttc cccttgggca catggtacga |
| 3301 | cctgcagacg gtgccaatag aggcccttgg cagcctccca cccccacctg cagctccccg |
| 3361 | tgagccagcc atccacagcg aggggcagtg ggtgacgctg ccggcccccc tggacaccat |
| 3421 | caacgtccac ctccgggctg ggtacatcat ccccctgcag ggccctggcc tcacaaccac |
| 3481 | agagtcccgc cagcagccca tggccctggc tgtggccctg accaagggtg gagaggcccg |
| 3541 | aggggagctg ttctgggacg atggagagag cctggaagtg ctggagcgag gggcctacac |
| 3601 | acaggtcatc ttcctggcca ggaataacac gatcgtgaat gagctggtac gtgtgaccag |
| 3661 | tgagggagct ggcctgcagc tgcagaaggt gactgtcctg ggcgtggcca cggcgcccca |
| 3721 | gcaggtcctc tccaacggtg tccctgtctc caacttcacc tacagccccg acaccaaggt |
| 3781 | cctggacatc tgtgtctcgc tgttgatggg agagcagttt ctcgtcagct ggtgttagcc |
| 3841 | gggcggagtg tgttagtctc tccagaggga ggctggttcc ccagggaagc agagcctgtg |
| 3901 | tgcgggcagc agctgtgtgc gggcctgggg gttgttaagt gcaattattt ttaataaaag |
| 3961 | gggcatttgg aaaaaaaaaa aaaaggtagc agtcgacaga tgaattctgc agatctgtgg |
| 4021 | cttctagctg cccgggtggc atccctgtga cccctcccca gtgcctctcc tggccctgga |
| 4081 | agttgccact ccagtgccca ccagccttgt cctaataaaa ttaagttgca tcattttgtc |
| 4141 | tgactaggtg tccttctata atattatggg gtggaggggg gtggtatgga gcaaggggca |
| 4201 | agttgggaag acaacctgag ttgttgggat tccaggcatc gagtagataa gtagcatggc |
| 4261 | gggttaatca ttaactacaa ggaaccccta gtgatggagt tggccactcc ctctctgcgc |
| 4321 | gctcgctcgc tcactgaggc cgggcgacca aaggtcgccc gacgcccggg ctttgcccgg |
| 4381 | gcggcctcag tgagcgagcg agcgcgcaga gagggacaga tccgaagccg ggcaaatcag |
| 4441 | cgcctggcag cagtggcgtc tggcggaaaa cctcagtgtg acgctccccg ccgcgtccca |
| 4501 | cgcttgttcc cggatctgac caccagcgaa atccgatttt tgcaccgagc tgggtaataa |
| 4561 | gcgttggcaa tttaaccgcc agtcaggctt tctttcacag tgtggattgg cgataaaaaa |
| 4621 | caactgctga cgccgctgcg cgatcagttc acccgttcac cgctggataa cgacttggcg |
| 4681 | taagtgaagc gacccgtaag accctaacgc ctgggtcgaa cgctggaagg cggcgggcca |
| 4741 | aaccaggccg aagcagcgtt gttgcagttc acggcagata cacttgctgt tgcggtgctg |
| 4801 | attacgaccg ctcactcgtg gcagcaacag gggaaaacct tatttatcag ccggaaaacc |
| 4861 | taccggattg ttggtagtgg tcaataggcg attaccgttg tgttgaagtg gcgagcgata |
| 4921 | caccgcttcc ggcgcggatt ggcctgaact gccaactggc gcaggtagca gagcgggtaa |
| 4981 | actggctcgg attagggccg caagaaaact atcccgaccg ccttactgcc gcctgttttg |
| 5041 | accgctggga tctgccaagt cagacagtat agcccgtacg tcttcccgag cgaaaacggt |
| 5101 | ctgcgctgcg ggacgcgcga attgaatttg gcccacacca gtggcgcggc gacttccagt |
| 5161 | tcaatatcag ccgctacagt gaacagcaac tgttggaaac cagccttcgc caactgctgc |
| 5221 | acgcggaaga aggcactggc tgaatatcga cggtttccag ttggggattg gtggcgacga |
| 5281 | ctcctggagc ccgtcagtat cggcggactt ccaactgagc gccggtcgct accttaccag |
| 5341 | ttggtctggt gtcaaaaagc gtccgcttga gtctagcgat cgcgcgcaga tctgtcatgt |
| 5401 | gagcaaaagg ccagcaaaag gccaggaacc gtaaaaaggc cgcgttgctg gcgtttttcc |
| 5461 | ataggctccg cccccctgac gagcatcaca aaaatcgacg ctcaagtcag aggtggcgaa |
| 5521 | acccgacagg actataaaga taccaggcgt ttccccctgg aagctccctc gtgcgctctc |
| 5581 | ctgttccgac cctgccgctt accggatacc tgtccgcctt tctcccttcg ggaagcgtgg |
| 5641 | cgctttctca tagctcacgc tgtaggtatc tcagttcggt gtaggtcgtt cgctccaagc |
| 5701 | tgggctgtgt gcacgaaccc cccgttcagc ccgaccgctg cgccttatcc ggtaactatc |
| 5761 | gtcttgagtc caacccggta agacacgact tatcgccact ggcagcagcc actggtaaca |
| 5821 | ggattagcag agcgaggtat gtaggcggtg ctacagagtt cttgaagtgg tggcctaact |
| 5881 | acggctacac tagaagaaca gtatttggta tctgcgctct gctgaagcca gttaccttcg |
| 5941 | gaaaaagagt tggtagctct tgatccggca aacaaaccac cgctggtagc ggtggttttt |
| 6001 | ttgtttgcaa gcagcagatt acgcgcagaa aaaaaggatc tcaagaagat cctttgatct |
| 6061 | tttctacggg gtctgacgct cagtggaacg aaaactcacg ttaagggatt ttggtcatga |
| 6121 | gattatcaaa aaggatcttc acctagatcc ttttcacgta gaaagccagt ccgcagaaac |
| 6181 | ggtgctgacc ccggatgaat gtcagctact gggctatctg gacaagggaa aacgcaagcg |
| 6241 | caaagagaaa gcaggtagct tgcagtgggc ttacatggcg atagctagac tgggcggttt |
| 6301 | tatggacagc aagcgaaccg gaattgccag ctggggcgcc ctctggtaag gttgggaagc |
| 6361 | cctgcaaagt aaactggatg gctttcttgc cgccaaggat ctgatggcgc aggggatcaa |
| 6421 | gatctgatca agagacagga tgaggatcgt ttcgcatgat tgaacaagat ggattgcacg |
| 6481 | caggttctcc ggccgcttgg gtggagaggc tattcggcta tgactgggca caacagacaa |
| 6541 | tcggctgctc tgatgccgcc gtgttccggc tgtcagcgca ggggcgcccg gttctttttg |
| 6601 | tcaagaccga cctgtccggt gccctgaatg aactgcaaga cgaggcagcg cggctatcgt |
| 6661 | ggctggccac gacgggcgtt ccttgcgcag ctgtgctcga cgttgtcact gaagcgggaa |
| 6721 | gggactggct gctattgggc gaagtgccgg ggcaggatct cctgtcatct caccttgctc |
| 6781 | ctgccgagaa agtatccatc atggctgatg caatgcggcg gctgcatacg cttgatccgg |
| 6841 | ctacctgccc attcgaccac caagcgaaac atcgcatcga gcgagcacgt actcggatgg |
| 6901 | aagccggtct tgtcgatcag gatgatctgg acgaagagca tcaggggctc gcgccagccg |
| 6961 | aactgttcgc caggctcaag gcgagcatgc ccgacggcga ggatctcgtc gtgacccatg |
| 7021 | gcgatgcctg cttgccgaat atcatggtgg aaaatggccg cttttctgga ttcatcgact |
| 7081 | gtggccggct gggtgtggcg gaccgctatc aggacatagc gttggctacc cgtgatattg |
| 7141 | ctgaagagct tggcggcgaa tgggctgacc gcttcctcgt gctttacggt atcgccgctc |
| 7201 | ccgattcgca gcgcatcgcc ttctatcgcc ttcttgacga gttcttctga atttaaagcc |
| 7261 | caatacgcaa accgcctctc cccgcgcgtt ggccgaagcg gttcaatatt ttgttaaaat |
| 7321 | tcgcgttaaa tttttgttaa atcagctatt ttttaaccaa taggccgaaa tcggcaaaat |
| 7381 | cccttgtaaa tcaaaagaat agaccgagat agggttgagt gttgttccag tttggaacaa |
| 7441 | gagtccacta ttaaagaacg tggactccaa cgtcaaaggg cgaaaaaccg tctatcaggg |
| 7501 | cgttggccca ctacgtgaac cttcacccta atcaagtttt ttggggtcga ggtgccgtaa |
| 7561 | agcactaaat cggaacccta aagggagccc ccgatttaga gcttgacggg gaaaccggcg |
| 7621 | aacgtggcga gaaaggaagg gaagaaagcg aaaggagcgg gcgctagggc gctggcaagt |
| 7681 | gtagcggtca cgctgcgcgt aaccaccaca cccgccgcgc taagcgccgc tacagggcgc |
| 7741 | gtcccttcgc cttcaggctg cgtcgagtac tgtactgtga gccagagttg cccggcgctc |
| 7801 | tccggctgcg gtagttcagg cagttcaatc aactgtttac cttgtggagc gactccagag |
| 7861 | gcacttcacc gcttgccagc ggcttacgat ccagcgccac gatccagtgc aggagatcgt |
| 7921 | tatcgctata cggaacaggt attcgctggt cacttcgata aggtttgccc ggataaacgg |
| 7981 | aactggaaaa actgctgctg gtgttttgct tccgtcagtg ctggatcggc gtgcggtcgg |
| 8041 | caaagaccag accgttctaa cagaactggc gattgttcgg cgtatcgcca aaatcaccgc |
| 8101 | cgtaagccga ccacgggttg ccgttttcag caggatttaa tcagcgactg atccacccag |
| 8161 | tcccagacga agccgccctg taaacgggga tactgacgaa acgcctgcca gtatttagcg |
| 8221 | aaaccgccaa gactgttacc caagcgtggg cgtattcgca aaggatcagc gggcgcgtct |
| 8281 | ctccaggtag cgaaagcctt ttttgatcga cctttcggca cagccgggaa gggctggtct |
| 8341 | tcaaccacgc gcgcgtacaa cgggcaaata atatcggtgg ccgtggtgtc ggctccgccg |
| 8401 | ccttcaactg caccgggcgg gaaggatcga cagatttgat ccagcgatac agcgcgtcgt |
| 8461 | gattagcgcc gtggcctgat tcaattcccc agcgaccagt agatcacact cgggtgatta |
| 8521 | cgattgcgct gcaccagtcg cgttacggtt cgctcttcgc cggtagccag cgcggatcac |
| 8581 | ggtcagacga ttcgttggca cgatccgtgg gtttcaatac tggcttcaaa ccaccactaa |
| 8641 | caggccgtag cggtcgcaca gcgtgtacca cagcggttgg ttcggataat cgaacagcgc |
| 8701 | acggcgttaa agttgttctg cttcaacagc aggatattct gcaccttcgt ctgctcttcc |
| 8761 | taacctgacc aagcagagga tctgctcgtg acggttaatc ctcgaatcag caacggcttg |
| 8821 | ccgttcagca gcagcagacc aagttcaatc cgcacctcgc ggaaaccgac aacgcaggct |
| 8881 | tctgcttcaa tcagcgtgcc gtcggcggtg tgcagttcaa ccaccgcacg atagagattc |
| 8941 | gggatttcgg cgctccacag tttcgggttt tcgacgttca gacgtagtgt gacgcgatct |
| 9001 | gcaaaccacc acgctcaacg ataatttcac cgccgaaagg cgcggtgccg ctggcgacct |
| 9061 | gcgtttcacc ctgccagaaa gaaactgtta cccgtaggta gtcacgcaac tcgccgcaca |
| 9121 | ctgaacttca gcctccagta cagcgcggct gaaatcgtct taaagcgagt ggcaactgga |
| 9181 | aatcgctgat ttgtgtagtc ggtttagcag caacgagact tcacggaaaa tccgctaatc |
| 9241 | cgccacagat cctgatcttc cagataactg ccgtcactcc aacgcagcac cttcaccgcg |
| 9301 | aggcggtttt ctccggcgcg taaaaatcgc tcaggtcaaa ttcagacggc aaacgactgt |
| 9361 | cctggccgta accgacccag cgcccgttgc accacagatt gaaacgccga gtttacgcct |
| 9421 | caaaaataat tcgcgtctgg ccttcctgta gccagctttc acaactataa tagtgagcga |
| 9481 | gtaacaaccc gtcggattct ccgtgggaac aaacggcgga ttgaccgtat agggataggt |
| 9541 | tacgttggtg tagtagggcg ctccgtaacc gtgctactgc cagtttgagg ggacgacgac |
| 9601 | agtatcggcc tcaggaagat cgcactccag ccagctttcc ggcaccgctt ctggtactgg |
| 9661 | aaaccaggca aagcgcctat cgcctatcag gctgcacaac tgttgggaag ggcgatctgt |
| 9721 | gcgggcctct tcgctattac gccagcttgc gaaagggggt agtgctgcaa ggcgattaag |
| 9781 | ttgggtaacg ccagggtttt cccagtcacg acgtggatct gggccactcc ctct |

| pP113 (SEQ ID NO: 505) | |
|---|---|
| (SEQ ID NO: 505) | |
| 1 | ctgcgcgctc gctcgctcac tgaggccgcc cgggcaaagc ccgggcgtcg ggcgaccttt |
| 61 | ggtcgcccgg cctcagtgag cgagcgagcg cgcagagagg gagtggccaa ctccatcact |
| 121 | aggggttcct ggaggggtgg agtcgtgacg tgaattacgt catagggtta gggaggtcgg |
| 181 | ccgctctagg agttaatttt taaaaagcag tcaaaagtcc aagtgccctt gcgagcattt |
| 241 | actctctctg tttgctctgg ttaataatct caggagcaca aacattcctt actagttcta |
| 301 | ggagttaatt tttaaaaagc agtcaaaagt ccaagtgccc ttgcgagcat ttactctctc |
| 361 | tgtttgctct ggttaataat ctcaggagca caaacattcc ttactagttc tagagcggcc |
| 421 | gccagtgtgc tggaattcgg cttttttagg gctggaagct acctttgaca tcatttcctc |
| 481 | tgcgaatgca tgtataattt ctacagaacc tattagaaag gatcacccag cctctgcttt |
| 541 | tgtacaactt tcccttaaaa aactgccaat cccactgctg tttggcccaa tagtgagaac |
| 601 | tttttcctgc tgcctcttgg tgcttttgcc tatggcccct attctgcctg ctgaagacac |
| 661 | tcttgccagc atggacttaa acccctccag ctctgacaat cctctttctc ttttgtttta |
| 721 | catgaagggt ctggcagcca aagcaatcac tcaaagttca aaccttatca ttttttgctt |
| 781 | tgttcctctt ggccttggtt ttgtacatca gctttgaaaa taccatccca gggttaatgc |
| 841 | tggggttaat ttataactga gagtgctcta gttctgcaat acaggacatg ctataaaaat |
| 901 | ggaaagatgt tgctttctga gagatcagct tacatgtggt accgagctcg gatccactag |
| 961 | taacggccgc cagtgtgctg gaattcggct ttccaggcca tctccaacca tgggagtgag |
| 1021 | gcacccgccc tgctcccacc ggctcctggc cgtctgcgcc ctcgtgtcct tggcaaccgc |
| 1081 | tatggagttt gggctgagct gggtctttct ggtggccctg ctgaagggag tccagtgtga |
| 1141 | gcagcaggga gccagcagac cagggccccg ggatgcccag gcacaccccg gccgtcccag |
| 1201 | agcagtgccc acacagtgcg acgtcccccc caacagccgc ttcgattgcg cccctgacaa |
| 1261 | ggccatcacc caggaacagt gcgaggcccg cggctgctgc tacatccctg caaagcaggg |
| 1321 | gctgcaggga gcccagatgg ggcagccctg gtgcttcttc ccacccagct accccagcta |
| 1381 | caagctggag aacctgagct cctctgaaat gggctacacg gccaccctga cccgtaccac |
| 1441 | ccccaccttc ttccccaagg acatcctgac cctgcggctg gacgtgatga tggagactga |
| 1501 | gaaccgcctc cacttcacga tcaaagatcc agctaacagg cgctacgagg tgcccttgga |
| 1561 | gaccccgcgt gtccacagcc gggcaccgtc cccactctac agcgtggagt tctccgagga |
| 1621 | gcccttcggg gtgatcgtgc accggcagct ggacggccgc gtgctgctga acacgacggt |
| 1681 | ggcgcccctg ttctttgcgg accagttcct tcagctgtcc acctcgctgc cctcgcagta |
| 1741 | tatcacaggc ctcgccgagc acctcagtcc cctgatgctc agcaccagct ggaccaggat |
| 1801 | caccctgtgg aaccgggacc ttgcgcccac gcccggtgcg aacctctacg ggtctcaccc |
| 1861 | tttctacctg gcgctggagg acggcgggtc ggcacacggg gtgttcctgc taaacagcaa |
| 1921 | tgccatggat gtggtcctgc agccgagccc tgcccttagc tggaggtcga caggtgggat |
| 1981 | cctggatgtc tacatcttcc tgggcccaga gcccaagagc gtggtgcagc agtacctgga |
| 2041 | cgttgtggga tacccgttca tgccgccata ctggggcctg ggcttccacc tgtgccgctg |
| 2101 | gggctactcc tccaccgcta tcacccgcca ggtggtggag aacatgacca gggcccactt |
| 2161 | ccccctggac gtccaatgga acgacctgga ctacatggac tcccggaggg acttcacgtt |
| 2221 | caacaaggat ggcttccggg acttcccggc catggtgcag gagctgcacc agggcggccg |
| 2281 | gcgctacatg atgatcgtgg atcctgccat cagcagctcg ggccctgccg ggagctacag |
| 2341 | gccctacgac gagggtctgc ggaggggggt tttcatcacc aacgagaccg gccagccgct |
| 2401 | gattgggaag gtatggcccg ggtccactgc cttccccgac ttcaccaacc ccacagccct |
| 2461 | ggcctggtgg gaggacatgg tggctgagtt ccatgaccag gtgcccttcg acggcatgtg |
| 2521 | gattgacatg aacgagcctt ccaacttcat cagaggctct gaggacggct gccccaacaa |
| 2581 | tgagctggag aacccaccct acgtgcctgg ggtggttggg gggaccctcc aggcggccac |
| 2641 | catctgtgcc tccagccacc agtttctctc cacacactac aacctgcaca acctctacgg |
| 2701 | cctgaccgaa gccatcgcct cccacagggc gctggtgaag gctcggggga cacgcccatt |
| 2761 | tgtgatctcc cgctcgacct ttgctggcca cggccgatac gccggccact ggacggggga |
| 2821 | cgtgtggagc tcctgggagc agctcgcctc ctccgtgcca gaaatcctgc agtttaacct |
| 2881 | gctgggggtg cctctggtcg gggccgacgt ctgcggcttc ctgggcaaca cctcagagga |
| 2941 | gctgtgtgtg cgctggaccc agctgggggc cttctacccc ttcatgcgga accacaacag |
| 3001 | cctgctcagt ctgccccagg agccgtacag cttcagcgag ccggcccagc aggccatgag |
| 3061 | gaaggccctc accctgcgct acgcactcct cccccacctc tacacactgt tccaccaggc |
| 3121 | ccacgtcgcg ggggagaccg tggcccggcc cctcttcctg gagttcccca aggactctag |
| 3181 | cacctggact gtggaccacc agctcctgtg gggggaggcc ctgctcatca ccccagtgct |
| 3241 | ccaggccggg aaggccgaag tgactggcta cttccccttg ggcacatggt acgacctgca |
| 3301 | gacggtgcca atagaggccc ttggcagcct cccaccccca cctgcagctc cccgtgagcc |
| 3361 | agccatccac agcgaggggc agtgggtgac gctgccggcc cccctggaca ccatcaacgt |
| 3421 | ccacctccgg gctgggtaca tcatccccct gcagggccct ggcctcacaa ccacagagtc |
| 3481 | ccgccagcag cccatggccc tggctgtggc cctgaccaag ggtggagagg cccgagggga |
| 3541 | gctgttctgg gacgatggag agagcctgga agtgctggag cgaggggcct acacacaggt |
| 3601 | catcttcctg gccaggaata acacgatcgt gaatgagctg gtacgtgtga ccagtgaggg |
| 3661 | agctggcctg cagctgcaga aggtgactgt cctgggcgtg gccacggcgc cccagcaggt |
| 3721 | cctctccaac ggtgtccctg tctccaactt cacctacagc cccgacacca aggtcctgga |
| 3781 | catctgtgtc tcgctgttga tgggagagca gtttctcgtc agctggtgtt agccgggcgg |
| 3841 | agtgtgttag tctctccaga gggaggctgg ttccccaggg aagcagagcc tgtgtgcggg |
| 3901 | cagcagctgt gtgcgggcct gggggttgtt aagtgcaatt atttttaata aaaggggcat |
| 3961 | ttggaaaaaa aaaaaaaagg tagcagtcga cagatgaatt ctgcagatct gtggcttcta |
| 4021 | gctgcccggg tggcatccct gtgacccctc cccagtgcct ctcctggccc tggaagttgc |
| 4081 | cactccagtg cccaccagcc ttgtcctaat aaaattaagt tgcatcattt tgtctgacta |
| 4141 | ggtgtccttc tataatatta tggggtggag gggggtggta tggagcaagg ggcaagttgg |
| 4201 | gaagacaacc tgagttgttg ggattccagg catcgagtag ataagtagca tggcgggtta |
| 4261 | atcattaact acaaggaacc cctagtgatg gagttggcca ctccctctct gcgcgctcgc |
| 4321 | tcgctcactg aggccgggcg accaaaggtc gcccgacgcc cgggctttgc ccgggcggcc |
| 4381 | tcagtgagcg agcgagcgcg cagagaggga cagatccgaa gccgggcaaa tcagcgcctg |
| 4441 | gcagcagtgg cgtctggcgg aaaacctcag tgtgacgctc cccgccgcgt cccacgcttg |
| 4501 | ttcccggatc tgaccaccag cgaaatccga tttttgcacc gagctgggta ataagcgttg |
| 4561 | gcaatttaac cgccagtcag gctttctttc acagtgtgga ttggcgataa aaaacaactg |
| 4621 | ctgacgccgc tgcgcgatca gttcacccgt tcaccgctgg ataacgactt ggcgtaagtg |
| 4681 | aagcgacccg taagacccta acgcctgggt cgaacgctgg aaggcggcgg gccaaaccag |
| 4741 | gccgaagcag cgttgttgca gttcacggca gatacacttg ctgttgcggt gctgattacg |
| 4801 | accgctcact cgtggcagca acaggggaaa accttattta tcagccggaa aacctaccgg |
| 4861 | attgttggta gtggtcaata ggcgattacc gttgtgttga agtggcgagc gatacaccgc |
| 4921 | ttccggcgcg gattggcctg aactgccaac tggcgcaggt agcagagcgg gtaaactggc |
| 4981 | tcggattagg gccgcaagaa aactatcccg accgccttac tgccgcctgt tttgaccgct |
| 5041 | gggatctgcc aagtcagaca gtatagcccg tacgtcttcc cgagcgaaaa cggtctgcgc |
| 5101 | tgcgggacgc gcgaattgaa tttggcccac accagtggcg cggcgacttc cagttcaata |
| 5161 | tcagccgcta cagtgaacag caactgttgg aaaccagcct tcgccaactg ctgcacgcgg |
| 5221 | aagaaggcac tggctgaata tcgacggttt ccagttgggg attggtggcg acgactcctg |
| 5281 | gagcccgtca gtatcggcgg acttccaact gagcgccggt cgctacctta ccagttggtc |
| 5341 | tggtgtcaaa aagcgtccgc ttgagtctag cgatcgcgcg cagatctgtc atgtgagcaa |
| 5401 | aaggccagca aaaggccagg aaccgtaaaa aggccgcgtt gctggcgttt ttccataggc |
| 5461 | tccgcccccc tgacgagcat cacaaaaatc gacgctcaag tcagaggtgg cgaaacccga |
| 5521 | caggactata aagataccag gcgtttcccc ctggaagctc cctcgtgcgc tctcctgttc |
| 5581 | cgaccctgcc gcttaccgga tacctgtccg cctttctccc ttcgggaagc gtggcgcttt |
| 5641 | ctcatagctc acgctgtagg tatctcagtt cggtgtaggt cgttcgctcc aagctgggct |
| 5701 | gtgtgcacga accccccgtt cagcccgacc gctgcgcctt atccggtaac tatcgtcttg |
| 5761 | agtccaaccc ggtaagacac gacttatcgc cactggcagc agccactggt aacaggatta |
| 5821 | gcagagcgag gtatgtaggc ggtgctacag agttcttgaa gtggtggcct aactacggct |
| 5881 | acactagaag aacagtattt ggtatctgcg ctctgctgaa gccagttacc ttcggaaaaa |
| 5941 | gagttggtag ctcttgatcc ggcaaacaaa ccaccgctgg tagcggtggt ttttttgttt |
| 6001 | gcaagcagca gattacgcgc agaaaaaaag gatctcaaga agatcctttg atcttttcta |
| 6061 | cggggtctga cgctcagtgg aacgaaaact cacgttaagg gattttggtc atgagattat |
| 6121 | caaaaaggat cttcacctag atccttttca cgtagaaagc cagtccgcag aaacggtgct |
| 6181 | gaccccggat gaatgtcagc tactgggcta tctggacaag ggaaaacgca agcgcaaaga |
| 6241 | gaaagcaggt agcttgcagt gggcttacat ggcgatagct agactgggcg gttttatgga |
| 6301 | cagcaagcga accggaattg ccagctgggg cgccctctgg taaggttggg aagccctgca |
| 6361 | aagtaaactg gatggctttc ttgccgccaa ggatctgatg gcgcagggga tcaagatctg |
| 6421 | atcaagagac aggatgagga tcgtttcgca tgattgaaca agatggattg cacgcaggtt |
| 6481 | ctccggccgc ttgggtggag aggctattcg gctatgactg ggcacaacag acaatcggct |
| 6541 | gctctgatgc cgccgtgttc cggctgtcag cgcaggggcg cccggttctt tttgtcaaga |
| 6601 | ccgacctgtc cggtgccctg aatgaactgc aagacgaggc agcgcggcta tcgtggctgg |
| 6661 | ccacgacggg cgttccttgc gcagctgtgc tcgacgttgt cactgaagcg ggaagggact |
| 6721 | ggctgctatt gggcgaagtg ccggggcagg atctcctgtc atctcacctt gctcctgccg |
| 6781 | agaaagtatc catcatggct gatgcaatgc ggcggctgca tacgcttgat ccggctacct |
| 6841 | gcccattcga ccaccaagcg aaacatcgca tcgagcgagc acgtactcgg atggaagccg |
| 6901 | gtcttgtcga tcaggatgat ctggacgaag agcatcaggg gctcgcgcca gccgaactgt |
| 6961 | tcgccaggct caaggcgagc atgcccgacg gcgaggatct cgtcgtgacc catggcgatg |
| 7021 | cctgcttgcc gaatatcatg gtggaaaatg gccgcttttc tggattcatc gactgtggcc |
| 7081 | ggctgggtgt ggcggaccgc tatcaggaca tagcgttggc tacccgtgat attgctgaag |
| 7141 | agcttggcgg cgaatgggct gaccgcttcc tcgtgcttta cggtatcgcc gctcccgatt |
| 7201 | cgcagcgcat cgccttctat cgccttcttg acgagttctt ctgaatttaa agcccaatac |
| 7261 | gcaaaccgcc tctccccgcg cgttggccga agcggttcaa tattttgtta aaattcgcgt |
| 7321 | taaatttttg ttaaatcagc tattttttaa ccaataggcc gaaatcggca aaatcccttg |
| 7381 | taaatcaaaa gaatagaccg agatagggtt gagtgttgtt ccagtttgga acaagagtcc |
| 7441 | actattaaag aacgtggact ccaacgtcaa agggcgaaaa accgtctatc agggcgttgg |
| 7501 | cccactacgt gaaccttcac cctaatcaag ttttttgggg tcgaggtgcc gtaaagcact |
| 7561 | aaatcggaac cctaaaggga gcccccgatt tagagcttga cggggaaacc ggcgaacgtg |
| 7621 | gcgagaaagg aagggaagaa agcgaaagga gcgggcgcta gggcgctggc aagtgtagcg |
| 7681 | gtcacgctgc gcgtaaccac cacacccgcc gcgctaagcg ccgctacagg gcgcgtccct |
| 7741 | tcgccttcag gctgcgtcga gtactgtact gtgagccaga gttgcccggc gctctccggc |
| 7801 | tgcggtagtt caggcagttc aatcaactgt ttaccttgtg gagcgactcc agaggcactt |
| 7861 | caccgcttgc cagcggctta cgatccagcg ccacgatcca gtgcaggaga tcgttatcgc |
| 7921 | tatacggaac aggtattcgc tggtcacttc gataaggttt gcccggataa acggaactgg |
| 7981 | aaaaactgct gctggtgttt tgcttccgtc agtgctggat cggcgtgcgg tcggcaaaga |
| 8041 | ccagaccgtt ctaacagaac tggcgattgt tcggcgtatc gccaaaatca ccgccgtaag |
| 8101 | ccgaccacgg gttgccgttt tcagcaggat ttaatcagcg actgatccac ccagtcccag |
| 8161 | acgaagccgc cctgtaaacg gggatactga cgaaacgcct gccagtattt agcgaaaccg |
| 8221 | ccaagactgt tacccaagcg tgggcgtatt cgcaaaggat cagcgggcgc gtctctccag |
| 8281 | gtagcgaaag ccttttttga tcgacctttc ggcacagccg ggaagggctg gtcttcaacc |
| 8341 | acgcgcgcgt acaacgggca aataatatcg gtggccgtgg tgtcggctcc gccgccttca |
| 8401 | actgcaccgg gcgggaagga tcgacagatt tgatccagcg atacagcgcg tcgtgattag |
| 8461 | cgccgtggcc tgattcaatt ccccagcgac cagtagatca cactcgggtg attacgattg |
| 8521 | cgctgcacca gtcgcgttac ggttcgctct tcgccggtag ccagcgcgga tcacggtcag |
| 8581 | acgattcgtt ggcacgatcc gtgggtttca atactggctt caaaccacca ctaacaggcc |
| 8641 | gtagcggtcg cacagcgtgt accacagcgg ttggttcgga taatcgaaca gcgcacggcg |
| 8701 | ttaaagttgt tctgcttcaa cagcaggata ttctgcacct tcgtctgctc ttcctaacct |
| 8761 | gaccaagcag aggatctgct cgtgacggtt aatcctcgaa tcagcaacgg cttgccgttc |
| 8821 | agcagcagca gaccaagttc aatccgcacc tcgcggaaac cgacaacgca ggcttctgct |
| 8881 | tcaatcagcg tgccgtcggc ggtgtgcagt tcaaccaccg cacgatagag attcgggatt |
| 8941 | tcggcgctcc acagtttcgg gttttcgacg ttcagacgta gtgtgacgcg atctgcaaac |
| 9001 | caccacgctc aacgataatt tcaccgccga aaggcgcggt gccgctggcg acctgcgttt |
| 9061 | caccctgcca gaaagaaact gttacccgta ggtagtcacg caactcgccg cacactgaac |
| 9121 | ttcagcctcc agtacagcgc ggctgaaatc gtcttaaagc gagtggcaac tggaaatcgc |
| 9181 | tgatttgtgt agtcggttta gcagcaacga gacttcacgg aaaatccgct aatccgccac |
| 9241 | agatcctgat cttccagata actgccgtca ctccaacgca gcaccttcac cgcgaggcgg |
| 9301 | ttttctccgg cgcgtaaaaa tcgctcaggt caaattcaga cggcaaacga ctgtcctggc |
| 9361 | cgtaaccgac ccagcgcccg ttgcaccaca gattgaaacg ccgagtttac gcctcaaaaa |
| 9421 | taattcgcgt ctggccttcc tgtagccagc tttcacaact ataatagtga gcgagtaaca |
| 9481 | acccgtcgga ttctccgtgg gaacaaacgg cggattgacc gtatagggat aggttacgtt |
| 9541 | ggtgtagtag ggcgctccgt aaccgtgcta ctgccagttt gaggggacga cgacagtatc |
| 9601 | ggcctcagga agatcgcact ccagccagct ttccggcacc gcttctggta ctggaaacca |
| 9661 | ggcaaagcgc ctatcgccta tcaggctgca caactgttgg gaagggcgat ctgtgcgggc |
| 9721 | ctcttcgcta ttacgccagc ttgcgaaagg gggtagtgct gcaaggcgat taagttgggt |
| 9781 | aacgccaggg ttttcccagt cacgacgtgg atctgggcca ctccctct |

| pP114 (SEQ ID NO: 506) | |
|---|---|
| (SEQ ID NO: 506) | |
| 1 | ctgcgcgctc gctcgctcac tgaggccgcc cgggcaaagc ccgggcgtcg ggcgaccttt |
| 61 | ggtcgcccgg cctcagtgag cgagcgagcg cgcagagagg gagtggccaa ctccatcact |
| 121 | aggggttcct ggaggggtgg agtcgtgacg tgaattacgt catagggtta gggaggtcgg |
| 181 | ccgctctagg agttaatttt taaaaagcag tcaaaagtcc aagtgccctt gcgagcattt |
| 241 | actctctctg tttgctctgg ttaataatct caggagcaca aacattcctt actagttcta |
| 301 | ggagttaatt tttaaaaagc agtcaaaagt ccaagtgccc ttgcgagcat ttactctctc |
| 361 | tgtttgctct ggttaataat ctcaggagca caaacattcc ttactagttc tagagcggcc |
| 421 | gccagtgtgc tggaattcgg cttttttagg gctggaagct acctttgaca tcatttcctc |
| 481 | tgcgaatgca tgtataattt ctacagaacc tattagaaag gatcacccag cctctgcttt |
| 541 | tgtacaactt tcccttaaaa aactgccaat cccactgctg tttggcccaa tagtgagaac |
| 601 | tttttcctgc tgcctcttgg tgcttttgcc tatggcccct attctgcctg ctgaagacac |
| 661 | tcttgccagc atggacttaa acccctccag ctctgacaat cctctttctc ttttgtttta |
| 721 | catgaagggt ctggcagcca aagcaatcac tcaaagttca aaccttatca ttttttgctt |
| 781 | tgttcctctt ggccttggtt ttgtacatca gctttgaaaa taccatccca gggttaatgc |
| 841 | tggggttaat ttataactga gagtgctcta gttctgcaat acaggacatg ctataaaaat |
| 901 | ggaaagatgt tgctttctga gagatcagct tacatgtggt accgagctcg gatccactag |
| 961 | taacggccgc cagtgtgctg gaattcggct ttccaggcca tctccaacca tggggtacag |
| 1021 | aatgcagctg ctgtcctgca ttgcactgtc actggcactg gttaccaact cacagcaggg |
| 1081 | agccagcaga ccagggcccc gggatgccca ggcacacccc ggccgtccca gagcagtgcc |
| 1141 | cacacagtgc gacgtccccc ccaacagccg cttcgattgc gcccctgaca aggccatcac |
| 1201 | ccaggaacag tgcgaggccc gcggctgctg ctacatccct gcaaagcagg ggctgcaggg |
| 1261 | agcccagatg gggcagccct ggtgcttctt cccacccagc taccccagct acaagctgga |
| 1321 | gaacctgagc tcctctgaaa tgggctacac ggccaccctg acccgtacca cccccacctt |
| 1381 | cttccccaag gacatcctga ccctgcggct ggacgtgatg atggagactg agaaccgcct |
| 1441 | ccacttcacg atcaaagatc cagctaacag gcgctacgag gtgcccttgg agaccccgcg |
| 1501 | tgtccacagc cgggcaccgt ccccactcta cagcgtggag ttctccgagg agcccttcgg |
| 1561 | ggtgatcgtg caccggcagc tggacggccg cgtgctgctg aacacgacgg tggcgcccct |
| 1621 | gttctttgcg gaccagttcc ttcagctgtc cacctcgctg ccctcgcagt atatcacagg |
| 1681 | cctcgccgag cacctcagtc ccctgatgct cagcaccagc tggaccagga tcaccctgtg |
| 1741 | gaaccgggac cttgcgccca cgcccggtgc gaacctctac gggtctcacc ctttctacct |
| 1801 | ggcgctggag gacggcgggt cggcacacgg ggtgttcctg ctaaacagca atgccatgga |
| 1861 | tgtggtcctg cagccgagcc ctgcccttag ctggaggtcg acaggtggga tcctggatgt |
| 1921 | ctacatcttc ctgggcccag agcccaagag cgtggtgcag cagtacctgg acgttgtggg |
| 1981 | atacccgttc atgccgccat actggggcct gggcttccac ctgtgccgct ggggctactc |
| 2041 | ctccaccgct atcacccgcc aggtggtgga gaacatgacc agggcccact tccccctgga |
| 2101 | cgtccaatgg aacgacctgg actacatgga ctcccggagg gacttcacgt tcaacaagga |
| 2161 | tggcttccgg gacttcccgg ccatggtgca ggagctgcac cagggcggcc ggcgctacat |
| 2221 | gatgatcgtg gatcctgcca tcagcagctc gggccctgcc gggagctaca ggccctacga |
| 2281 | cgagggtctg cggagggggg ttttcatcac caacgagacc ggccagccgc tgattgggaa |
| 2341 | ggtatggccc gggtccactg ccttccccga cttcaccaac cccacagccc tggcctggtg |
| 2401 | ggaggacatg gtggctgagt tccatgacca ggtgcccttc gacggcatgt ggattgacat |
| 2461 | gaacgagcct tccaacttca tcagaggctc tgaggacggc tgccccaaca atgagctgga |
| 2521 | gaacccaccc tacgtgcctg gggtggttgg ggggaccctc caggcggcca ccatctgtgc |
| 2581 | ctccagccac cagtttctct ccacacacta caacctgcac aacctctacg gcctgaccga |
| 2641 | agccatcgcc tcccacaggg cgctggtgaa ggctcggggg acacgcccat ttgtgatctc |
| 2701 | ccgctcgacc tttgctggcc acggccgata cgccggccac tggacggggg acgtgtggag |
| 2761 | ctcctgggag cagctcgcct cctccgtgcc agaaatcctg cagtttaacc tgctgggggt |
| 2821 | gcctctggtc ggggccgacg tctgcggctt cctgggcaac acctcagagg agctgtgtgt |
| 2881 | gcgctggacc cagctggggg ccttctaccc cttcatgcgg aaccacaaca gcctgctcag |
| 2941 | tctgccccag gagccgtaca gcttcagcga gccggcccag caggccatga ggaaggccct |
| 3001 | caccctgcgc tacgcactcc tcccccacct ctacacactg ttccaccagg cccacgtcgc |
| 3061 | gggggagacc gtggcccggc ccctcttcct ggagttcccc aaggactcta gcacctggac |
| 3121 | tgtggaccac cagctcctgt ggggggaggc cctgctcatc accccagtgc tccaggccgg |
| 3181 | gaaggccgaa gtgactggct acttcccctt gggcacatgg tacgacctgc agacggtgcc |
| 3241 | aatagaggcc cttggcagcc tcccaccccc acctgcagct ccccgtgagc cagccatcca |
| 3301 | cagcgagggg cagtgggtga cgctgccggc ccccctggac accatcaacg tccacctccg |
| 3361 | ggctgggtac atcatccccc tgcagggccc tggcctcaca accacagagt cccgccagca |
| 3421 | gcccatggcc ctggctgtgg ccctgaccaa gggtggagag gcccgagggg agctgttctg |
| 3481 | ggacgatgga gagagcctgg aagtgctgga gcgaggggcc tacacacagg tcatcttcct |
| 3541 | ggccaggaat aacacgatcg tgaatgagct ggtacgtgtg accagtgagg gagctggcct |
| 3601 | gcagctgcag aaggtgactg tcctgggcgt ggccacggcg ccccagcagg tcctctccaa |
| 3661 | cggtgtccct gtctccaact tcacctacag ccccgacacc aaggtcctgg acatctgtgt |
| 3721 | ctcgctgttg atgggagagc agtttctcgt cagctggtgt tagccgggcg gagtgtgtta |
| 3781 | gtctctccag agggaggctg gttccccagg gaagcagagc ctgtgtgcgg gcagcagctg |
| 3841 | tgtgcgggcc tgggggttgt taagtgcaat tatttttaat aaaaggggca tttggaaaaa |
| 3901 | aaaaaaaaag gtagcagtcg acagatgaat tctgcagatc tgtggcttct agctgcccgg |
| 3961 | gtggcatccc tgtgacccct ccccagtgcc tctcctggcc ctggaagttg ccactccagt |
| 4021 | gcccaccagc cttgtcctaa taaaattaag ttgcatcatt ttgtctgact aggtgtcctt |
| 4081 | ctataatatt atggggtgga ggggggtggt atggagcaag gggcaagttg ggaagacaac |
| 4141 | ctgagttgtt gggattccag gcatcgagta gataagtagc atggcgggtt aatcattaac |
| 4201 | tacaaggaac ccctagtgat ggagttggcc actccctctc tgcgcgctcg ctcgctcact |
| 4261 | gaggccgggc gaccaaaggt cgcccgacgc ccgggctttg cccgggcggc ctcagtgagc |
| 4321 | gagcgagcgc gcagagaggg acagatccga agccgggcaa atcagcgcct ggcagcagtg |
| 4381 | gcgtctggcg gaaaacctca gtgtgacgct ccccgccgcg tcccacgctt gttcccggat |
| 4441 | ctgaccacca gcgaaatccg atttttgcac cgagctgggt aataagcgtt ggcaatttaa |
| 4501 | ccgccagtca ggctttcttt cacagtgtgg attggcgata aaaaacaact gctgacgccg |
| 4561 | ctgcgcgatc agttcacccg ttcaccgctg gataacgact tggcgtaagt gaagcgaccc |
| 4621 | gtaagaccct aacgcctggg tcgaacgctg gaaggcggcg ggccaaacca ggccgaagca |
| 4681 | gcgttgttgc agttcacggc agatacactt gctgttgcgg tgctgattac gaccgctcac |
| 4741 | tcgtggcagc aacaggggaa aaccttattt atcagccgga aaacctaccg gattgttggt |
| 4801 | agtggtcaat aggcgattac cgttgtgttg aagtggcgag cgatacaccg cttccggcgc |
| 4861 | ggattggcct gaactgccaa ctggcgcagg tagcagagcg ggtaaactgg ctcggattag |
| 4921 | ggccgcaaga aaactatccc gaccgcctta ctgccgcctg ttttgaccgc tgggatctgc |
| 4981 | caagtcagac agtatagccc gtacgtcttc ccgagcgaaa acggtctgcg ctgcgggacg |
| 5041 | cgcgaattga atttggccca caccagtggc gcggcgactt ccagttcaat atcagccgct |
| 5101 | acagtgaaca gcaactgttg gaaaccagcc ttcgccaact gctgcacgcg gaagaaggca |
| 5161 | ctggctgaat atcgacggtt tccagttggg gattggtggc gacgactcct ggagcccgtc |
| 5221 | agtatcggcg gacttccaac tgagcgccgg tcgctacctt accagttggt ctggtgtcaa |
| 5281 | aaagcgtccg cttgagtcta gcgatcgcgc gcagatctgt catgtgagca aaaggccagc |
| 5341 | aaaaggccag gaaccgtaaa aaggccgcgt tgctggcgtt tttccatagg ctccgccccc |
| 5401 | ctgacgagca tcacaaaaat cgacgctcaa gtcagaggtg gcgaaacccg acaggactat |
| 5461 | aaagatacca ggcgtttccc cctggaagct ccctcgtgcg ctctcctgtt ccgaccctgc |
| 5521 | cgcttaccgg atacctgtcc gcctttctcc cttcgggaag cgtggcgctt tctcatagct |
| 5581 | cacgctgtag gtatctcagt tcggtgtagg tcgttcgctc caagctgggc tgtgtgcacg |
| 5641 | aaccccccgt tcagcccgac cgctgcgcct tatccggtaa ctatcgtctt gagtccaacc |
| 5701 | cggtaagaca cgacttatcg ccactggcag cagccactgg taacaggatt agcagagcga |
| 5761 | ggtatgtagg cggtgctaca gagttcttga agtggtggcc taactacggc tacactagaa |
| 5821 | gaacagtatt tggtatctgc gctctgctga agccagttac cttcggaaaa agagttggta |
| 5881 | gctcttgatc cggcaaacaa accaccgctg gtagcggtgg tttttttgtt tgcaagcagc |
| 5941 | agattacgcg cagaaaaaaa ggatctcaag aagatccttt gatcttttct acggggtctg |
| 6001 | acgctcagtg gaacgaaaac tcacgttaag ggattttggt catgagatta tcaaaaagga |
| 6061 | tcttcaccta gatccttttc acgtagaaag ccagtccgca gaaacggtgc tgaccccgga |
| 6121 | tgaatgtcag ctactgggct atctggacaa gggaaaacgc aagcgcaaag agaaagcagg |
| 6181 | tagcttgcag tgggcttaca tggcgatagc tagactgggc ggttttatgg acagcaagcg |
| 6241 | aaccggaatt gccagctggg gcgccctctg gtaaggttgg gaagccctgc aaagtaaact |
| 6301 | ggatggcttt cttgccgcca aggatctgat ggcgcagggg atcaagatct gatcaagaga |
| 6361 | caggatgagg atcgtttcgc atgattgaac aagatggatt gcacgcaggt tctccggccg |
| 6421 | cttgggtgga gaggctattc ggctatgact gggcacaaca gacaatcggc tgctctgatg |
| 6481 | ccgccgtgtt ccggctgtca gcgcaggggc gcccggttct ttttgtcaag accgacctgt |
| 6541 | ccggtgccct gaatgaactg caagacgagg cagcgcggct atcgtggctg gccacgacgg |
| 6601 | gcgttccttg cgcagctgtg ctcgacgttg tcactgaagc gggaagggac tggctgctat |
| 6661 | tgggcgaagt gccggggcag gatctcctgt catctcacct tgctcctgcc gagaaagtat |
| 6721 | ccatcatggc tgatgcaatg cggcggctgc atacgcttga tccggctacc tgcccattcg |
| 6781 | accaccaagc gaaacatcgc atcgagcgag cacgtactcg gatggaagcc ggtcttgtcg |
| 6841 | atcaggatga tctggacgaa gagcatcagg ggctcgcgcc agccgaactg ttcgccaggc |
| 6901 | tcaaggcgag catgcccgac ggcgaggatc tcgtcgtgac ccatggcgat gcctgcttgc |
| 6961 | cgaatatcat ggtggaaaat ggccgctttt ctggattcat cgactgtggc cggctgggtg |
| 7021 | tggcggaccg ctatcaggac atagcgttgg ctacccgtga tattgctgaa gagcttggcg |
| 7081 | gcgaatgggc tgaccgcttc ctcgtgcttt acggtatcgc cgctcccgat tcgcagcgca |
| 7141 | tcgccttcta tcgccttctt gacgagttct tctgaattta aagcccaata cgcaaaccgc |
| 7201 | ctctccccgc gcgttggccg aagcggttca atattttgtt aaaattcgcg ttaaattttt |
| 7261 | gttaaatcag ctatttttta accaataggc cgaaatcggc aaaatccctt gtaaatcaaa |
| 7321 | agaatagacc gagatagggt tgagtgttgt tccagtttgg aacaagagtc cactattaaa |
| 7381 | gaacgtggac tccaacgtca aagggcgaaa aaccgtctat cagggcgttg gcccactacg |
| 7441 | tgaaccttca ccctaatcaa gttttttggg gtcgaggtgc cgtaaagcac taaatcggaa |
| 7501 | ccctaaaggg agcccccgat ttagagcttg acggggaaac cggcgaacgt ggcgagaaag |
| 7561 | gaagggaaga aagcgaaagg agcgggcgct agggcgctgg caagtgtagc ggtcacgctg |
| 7621 | cgcgtaacca ccacacccgc cgcgctaagc gccgctacag ggcgcgtccc ttcgccttca |
| 7681 | ggctgcgtcg agtactgtac tgtgagccag agttgcccgg cgctctccgg ctgcggtagt |
| 7741 | tcaggcagtt caatcaactg tttaccttgt ggagcgactc cagaggcact tcaccgcttg |
| 7801 | ccagcggctt acgatccagc gccacgatcc agtgcaggag atcgttatcg ctatacggaa |
| 7861 | caggtattcg ctggtcactt cgataaggtt tgcccggata aacggaactg gaaaaactgc |
| 7921 | tgctggtgtt ttgcttccgt cagtgctgga tcggcgtgcg gtcggcaaag accagaccgt |
| 7981 | tctaacagaa ctggcgattg ttcggcgtat cgccaaaatc accgccgtaa gccgaccacg |
| 8041 | ggttgccgtt ttcagcagga tttaatcagc gactgatcca cccagtccca gacgaagccg |
| 8101 | ccctgtaaac ggggatactg acgaaacgcc tgccagtatt tagcgaaacc gccaagactg |
| 8161 | ttacccaagc gtgggcgtat tcgcaaagga tcagcgggcg cgtctctcca ggtagcgaaa |
| 8221 | gccttttttg atcgaccttt cggcacagcc gggaagggct ggtcttcaac cacgcgcgcg |
| 8281 | tacaacgggc aaataatatc ggtggccgtg gtgtcggctc cgccgccttc aactgcaccg |
| 8341 | ggcgggaagg atcgacagat ttgatccagc gatacagcgc gtcgtgatta gcgccgtggc |
| 8401 | ctgattcaat tccccagcga ccagtagatc acactcgggt gattacgatt gcgctgcacc |
| 8461 | agtcgcgtta cggttcgctc ttcgccggta gccagcgcgg atcacggtca gacgattcgt |
| 8521 | tggcacgatc cgtgggtttc aatactggct tcaaaccacc actaacaggc cgtagcggtc |
| 8581 | gcacagcgtg taccacagcg gttggttcgg ataatcgaac agcgcacggc gttaaagttg |
| 8641 | ttctgcttca acagcaggat attctgcacc ttcgtctgct cttcctaacc tgaccaagca |
| 8701 | gaggatctgc tcgtgacggt taatcctcga atcagcaacg gcttgccgtt cagcagcagc |
| 8761 | agaccaagtt caatccgcac ctcgcggaaa ccgacaacgc aggcttctgc ttcaatcagc |
| 8821 | gtgccgtcgg cggtgtgcag ttcaaccacc gcacgataga gattcgggat ttcggcgctc |
| 8881 | cacagtttcg ggttttcgac gttcagacgt agtgtgacgc gatctgcaaa ccaccacgct |
| 8941 | caacgataat ttcaccgccg aaaggcgcgg tgccgctggc gacctgcgtt tcaccctgcc |
| 9001 | agaaagaaac tgttacccgt aggtagtcac gcaactcgcc gcacactgaa cttcagcctc |
| 9061 | cagtacagcg cggctgaaat cgtcttaaag cgagtggcaa ctggaaatcg ctgatttgtg |
| 9121 | tagtcggttt agcagcaacg agacttcacg gaaaatccgc taatccgcca cagatcctga |
| 9181 | tcttccagat aactgccgtc actccaacgc agcaccttca ccgcgaggcg gttttctccg |
| 9241 | gcgcgtaaaa atcgctcagg tcaaattcag acggcaaacg actgtcctgg ccgtaaccga |
| 9301 | cccagcgccc gttgcaccac agattgaaac gccgagttta cgcctcaaaa ataattcgcg |
| 9361 | tctggccttc ctgtagccag ctttcacaac tataatagtg agcgagtaac aacccgtcgg |
| 9421 | attctccgtg ggaacaaacg gcggattgac cgtataggga taggttacgt tggtgtagta |
| 9481 | gggcgctccg taaccgtgct actgccagtt tgaggggacg acgacagtat cggcctcagg |
| 9541 | aagatcgcac tccagccagc tttccggcac cgcttctggt actggaaacc aggcaaagcg |
| 9601 | cctatcgcct atcaggctgc acaactgttg ggaagggcga tctgtgcggg cctcttcgct |
| 9661 | attacgccag cttgcgaaag ggggtagtgc tgcaaggcga ttaagttggg taacgccagg |
| 9721 | gttttcccag tcacgacgtg gatctgggcc actccctct |

| pP150 (SEQ ID NO: 507) | |
|---|---|
| (SEQ ID NO: 507) | |
| 1 | ctgcgcgctc gctcgctcac tgaggccgcc cgggcaaagc ccgggcgtcg ggcgaccttt |
| 61 | ggtcgcccgg cctcagtgag cgagcgagcg cgcagagagg gagtggccaa ctccatcact |
| 121 | aggggttcct ggaggggtgg agtcgtgacg tgaattacgt catagggtta gggaggtcgg |
| 181 | ccgctctagg agttaatttt taaaaagcag tcaaaagtcc aagtgccctt gcgagcattt |
| 241 | actctctctg tttgctctgg ttaataatct caggagcaca aacattcctt actagttcta |
| 301 | ggagttaatt tttaaaaagc agtcaaaagt ccaagtgccc ttgcgagcat ttactctctc |
| 361 | tgtttgctct ggttaataat ctcaggagca caaacattcc ttactagttc tagagcggcc |
| 421 | gccagtgtgc tggaattcgg cttttttagg gctggaagct acctttgaca tcatttcctc |
| 481 | tgcgaatgca tgtataattt ctacagaacc tattagaaag gatcacccag cctctgcttt |
| 541 | tgtacaactt tcccttaaaa aactgccaat cccactgctg tttggcccaa tagtgagaac |
| 601 | tttttcctgc tgcctcttgg tgcttttgcc tatggcccct attctgcctg ctgaagacac |
| 661 | tcttgccagc atggacttaa acccctccag ctctgacaat cctctttctc ttttgtttta |
| 721 | catgaagggt ctggcagcca aagcaatcac tcaaagttca aaccttatca ttttttgctt |
| 781 | tgttcctctt ggccttggtt ttgtacatca gctttgaaaa taccatccca gggttaatgc |
| 841 | tggggttaat ttataactga gagtgctcta gttctgcaat acaggacatg ctataaaaat |
| 901 | ggaaagatgt tgctttctga gagatcagct tacatgtggt accgagctcg gatccactag |
| 961 | taacggccgc cagtgtgctg gaattcggct ttccaggcca tcttttttta tgggagtgag |
| 1021 | gcacccgccc tgctcccacc ggctcctggc cgtctgcgcc ctcgtgtcct tggcaaccgc |
| 1081 | catggagttt gggctgagct gggtctttct ggtggccctg ctgaagggag tccagtgtga |
| 1141 | gcagcaggga gccagcagac cagggccccg ggatgcccag gcacaccccg gccgtcccag |
| 1201 | agcagtgccc acacagtgcg acgtcccccc caacagccgc ttcgattgcg cccctgacaa |
| 1261 | ggccatcacc caggaacagt gcgaggcccg cggctgctgc tacatccctg caaagcaggg |
| 1321 | gctgcaggga gcccagatgg ggcagccctg gtgcttcttc ccacccagct accccagcta |
| 1381 | caagctggag aacctgagct cctctgaaat gggctacacg gccaccctga cccgtaccac |
| 1441 | ccccaccttc ttccccaagg acatcctgac cctgcggctg gacgtgatga tggagactga |
| 1501 | gaaccgcctc cacttcacga tcaaagatcc agctaacagg cgctacgagg tgcccttgga |
| 1561 | gaccccgcgt gtccacagcc gggcaccgtc cccactctac agcgtggagt tctccgagga |
| 1621 | gcccttcggg gtgatcgtgc accggcagct ggacggccgc gtgctgctga acacgacggt |
| 1681 | ggcgcccctg ttctttgcgg accagttcct tcagctgtcc acctcgctgc cctcgcagta |
| 1741 | tatcacaggc ctcgccgagc acctcagtcc cctgatgctc agcaccagct ggaccaggat |
| 1801 | caccctgtgg aaccgggacc ttgcgcccac gcccggtgcg aacctctacg ggtctcaccc |
| 1861 | tttctacctg gcgctggagg acggcgggtc ggcacacggg gtgttcctgc taaacagcaa |
| 1921 | tgccatggat gtggtcctgc agccgagccc tgcccttagc tggaggtcga caggtgggat |
| 1981 | cctggatgtc tacatcttcc tgggcccaga gcccaagagc gtggtgcagc agtacctgga |
| 2041 | cgttgtggga tacccgttca tgccgccata ctggggcctg ggcttccacc tgtgccgctg |
| 2101 | gggctactcc tccaccgcta tcacccgcca ggtggtggag aacatgacca gggcccactt |
| 2161 | ccccctggac gtccaatgga acgacctgga ctacatggac tcccggaggg acttcacgtt |
| 2221 | caacaaggat ggcttccggg acttcccggc catggtgcag gagctgcacc agggcggccg |
| 2281 | gcgctacatg atgatcgtgg atcctgccat cagcagctcg ggccctgccg ggagctacag |
| 2341 | gccctacgac gagggtctgc ggaggggggt tttcatcacc aacgagaccg gccagccgct |
| 2401 | gattgggaag gtatggcccg ggtccactgc cttccccgac ttcaccaacc ccacagccct |
| 2461 | ggcctggtgg gaggacatgg tggctgagtt ccatgaccag gtgcccttcg acggcatgtg |
| 2521 | gattgacatg aacgagcctt ccaacttcat cagaggctct gaggacggct gccccaacaa |
| 2581 | tgagctggag aacccaccct acgtgcctgg ggtggttggg gggaccctcc aggcggccac |
| 2641 | catctgtgcc tccagccacc agtttctctc cacacactac aacctgcaca acctctacgg |
| 2701 | cctgaccgaa gccatcgcct cccacagggc gctggtgaag gctcggggga cacgcccatt |
| 2761 | tgtgatctcc cgctcgacct ttgctggcca cggccgatac gccggccact ggacggggga |
| 2821 | cgtgtggagc tcctgggagc agctcgcctc ctccgtgcca gaaatcctgc agtttaacct |
| 2881 | gctgggggtg cctctggtcg gggccgacgt ctgcggcttc ctgggcaaca cctcagagga |
| 2941 | gctgtgtgtg cgctggaccc agctgggggc cttctacccc ttcatgcgga accacaacag |
| 3001 | cctgctcagt ctgccccagg agccgtacag cttcagcgag ccggcccagc aggccatgag |
| 3061 | gaaggccctc accctgcgct acgcactcct cccccacctc tacacactgt tccaccaggc |
| 3121 | ccacgtcgcg ggggagaccg tggcccggcc cctcttcctg gagttcccca aggactctag |
| 3181 | cacctggact gtggaccacc agctcctgtg gggggaggcc ctgctcatca ccccagtgct |
| 3241 | ccaggccggg aaggccgaag tgactggcta cttccccttg ggcacatggt acgacctgca |
| 3301 | gacggtgcca atagaggccc ttggcagcct cccaccccca cctgcagctc cccgtgagcc |
| 3361 | agccatccac agcgaggggc agtgggtgac gctgccggcc cccctggaca ccatcaacgt |
| 3421 | ccacctccgg gctgggtaca tcatccccct gcagggccct ggcctcacaa ccacagagtc |
| 3481 | ccgccagcag cccatggccc tggctgtggc cctgaccaag ggtggagagg cccgagggga |
| 3541 | gctgttctgg gacgatggag agagcctgga agtgctggag cgaggggcct acacacaggt |
| 3601 | catcttcctg gccaggaata acacgatcgt gaatgagctg gtacgtgtga ccagtgaggg |
| 3661 | agctggcctg cagctgcaga aggtgactgt cctgggcgtg gccacggcgc cccagcaggt |
| 3721 | cctctccaac ggtgtccctg tctccaactt cacctacagc cccgacacca aggtcctgga |
| 3781 | catctgtgtc tcgctgttga tgggagagca gtttctcgtc agctggtgtt agccgggcgg |
| 3841 | agtgtgttag tctctccaga gggaggctgg ttccccaggg aagcagagcc tgtgtgcggg |
| 3901 | cagcagctgt gtgcgggcct gggggttgtt aagtgcaatt atttttaata aaaggggcat |
| 3961 | ttggaaaaaa aaaaaaaagg tagcagtcga cagatgaatt ctgcagatct gtggcttcta |
| 4021 | gctgcccggg tggcatccct gtgacccctc cccagtgcct ctcctggccc tggaagttgc |
| 4081 | cactccagtg cccaccagcc ttgtcctaat aaaattaagt tgcatcattt tgtctgacta |
| 4141 | ggtgtccttc tataatatta tggggtggag gggggtggta tggagcaagg ggcaagttgg |
| 4201 | gaagacaacc tgagttgttg ggattccagg catcgagtag ataagtagca tggcgggtta |
| 4261 | atcattaact acaaggaacc cctagtgatg gagttggcca ctccctctct gcgcgctcgc |
| 4321 | tcgctcactg aggccgggcg accaaaggtc gcccgacgcc cgggctttgc ccgggcggcc |
| 4381 | tcagtgagcg agcgagcgcg cagagaggga cagatccgaa gccgggcaaa tcagcgcctg |
| 4441 | gcagcagtgg cgtctggcgg aaaacctcag tgtgacgctc cccgccgcgt cccacgcttg |
| 4501 | ttcccggatc tgaccaccag cgaaatccga tttttgcacc gagctgggta ataagcgttg |
| 4561 | gcaatttaac cgccagtcag gctttctttc acagtgtgga ttggcgataa aaaacaactg |
| 4621 | ctgacgccgc tgcgcgatca gttcacccgt tcaccgctgg ataacgactt ggcgtaagtg |
| 4681 | aagcgacccg taagacccta acgcctgggt cgaacgctgg aaggcggcgg gccaaaccag |
| 4741 | gccgaagcag cgttgttgca gttcacggca gatacacttg ctgttgcggt gctgattacg |
| 4801 | accgctcact cgtggcagca acaggggaaa accttattta tcagccggaa aacctaccgg |
| 4861 | attgttggta gtggtcaata ggcgattacc gttgtgttga agtggcgagc gatacaccgc |
| 4921 | ttccggcgcg gattggcctg aactgccaac tggcgcaggt agcagagcgg gtaaactggc |
| 4981 | tcggattagg gccgcaagaa aactatcccg accgccttac tgccgcctgt tttgaccgct |
| 5041 | gggatctgcc aagtcagaca gtatagcccg tacgtcttcc cgagcgaaaa cggtctgcgc |
| 5101 | tgcgggacgc gcgaattgaa tttggcccac accagtggcg cggcgacttc cagttcaata |
| 5161 | tcagccgcta cagtgaacag caactgttgg aaaccagcct tcgccaactg ctgcacgcgg |
| 5221 | aagaaggcac tggctgaata tcgacggttt ccagttgggg attggtggcg acgactcctg |
| 5281 | gagcccgtca gtatcggcgg acttccaact gagcgccggt cgctacctta ccagttggtc |
| 5341 | tggtgtcaaa aagcgtccgc ttgagtctag cgatcgcgcg cagatctgtc atgtgagcaa |
| 5401 | aaggccagca aaaggccagg aaccgtaaaa aggccgcgtt gctggcgttt ttccataggc |
| 5461 | tccgcccccc tgacgagcat cacaaaaatc gacgctcaag tcagaggtgg cgaaacccga |
| 5521 | caggactata aagataccag gcgtttcccc ctggaagctc cctcgtgcgc tctcctgttc |
| 5581 | cgaccctgcc gcttaccgga tacctgtccg cctttctccc ttcgggaagc gtggcgcttt |
| 5641 | ctcatagctc acgctgtagg tatctcagtt cggtgtaggt cgttcgctcc aagctgggct |
| 5701 | gtgtgcacga accccccgtt cagcccgacc gctgcgcctt atccggtaac tatcgtcttg |
| 5761 | agtccaaccc ggtaagacac gacttatcgc cactggcagc agccactggt aacaggatta |
| 5821 | gcagagcgag gtatgtaggc ggtgctacag agttcttgaa gtggtggcct aactacggct |
| 5881 | acactagaag aacagtattt ggtatctgcg ctctgctgaa gccagttacc ttcggaaaaa |
| 5941 | gagttggtag ctcttgatcc ggcaaacaaa ccaccgctgg tagcggtggt ttttttgttt |
| 6001 | gcaagcagca gattacgcgc agaaaaaaag gatctcaaga agatcctttg atcttttcta |
| 6061 | cggggtctga cgctcagtgg aacgaaaact cacgttaagg gattttggtc atgagattat |
| 6121 | caaaaaggat cttcacctag atccttttca cgtagaaagc cagtccgcag aaacggtgct |
| 6181 | gaccccggat gaatgtcagc tactgggcta tctggacaag ggaaaacgca agcgcaaaga |
| 6241 | gaaagcaggt agcttgcagt gggcttacat ggcgatagct agactgggcg gttttatgga |
| 6301 | cagcaagcga accggaattg ccagctgggg cgccctctgg taaggttggg aagccctgca |
| 6361 | aagtaaactg gatggctttc ttgccgccaa ggatctgatg gcgcagggga tcaagatctg |
| 6421 | atcaagagac aggatgagga tcgtttcgca tgattgaaca agatggattg cacgcaggtt |
| 6481 | ctccggccgc ttgggtggag aggctattcg gctatgactg ggcacaacag acaatcggct |
| 6541 | gctctgatgc cgccgtgttc cggctgtcag cgcaggggcg cccggttctt tttgtcaaga |
| 6601 | ccgacctgtc cggtgccctg aatgaactgc aagacgaggc agcgcggcta tcgtggctgg |
| 6661 | ccacgacggg cgttccttgc gcagctgtgc tcgacgttgt cactgaagcg ggaagggact |
| 6721 | ggctgctatt gggcgaagtg ccggggcagg atctcctgtc atctcacctt gctcctgccg |
| 6781 | agaaagtatc catcatggct gatgcaatgc ggcggctgca tacgcttgat ccggctacct |
| 6841 | gcccattcga ccaccaagcg aaacatcgca tcgagcgagc acgtactcgg atggaagccg |
| 6901 | gtcttgtcga tcaggatgat ctggacgaag agcatcaggg gctcgcgcca gccgaactgt |
| 6961 | tcgccaggct caaggcgagc atgcccgacg gcgaggatct cgtcgtgacc catggcgatg |
| 7021 | cctgcttgcc gaatatcatg gtggaaaatg gccgcttttc tggattcatc gactgtggcc |
| 7081 | ggctgggtgt ggcggaccgc tatcaggaca tagcgttggc tacccgtgat attgctgaag |
| 7141 | agcttggcgg cgaatgggct gaccgcttcc tcgtgcttta cggtatcgcc gctcccgatt |
| 7201 | cgcagcgcat cgccttctat cgccttcttg acgagttctt ctgaatttaa agcccaatac |
| 7261 | gcaaaccgcc tctccccgcg cgttggccga agcggttcaa tattttgtta aaattcgcgt |
| 7321 | taaatttttg ttaaatcagc tattttttaa ccaataggcc gaaatcggca aaatcccttg |
| 7381 | taaatcaaaa gaatagaccg agatagggtt gagtgttgtt ccagtttgga acaagagtcc |
| 7441 | actattaaag aacgtggact ccaacgtcaa agggcgaaaa accgtctatc agggcgttgg |
| 7501 | cccactacgt gaaccttcac cctaatcaag ttttttgggg tcgaggtgcc gtaaagcact |
| 7561 | aaatcggaac cctaaaggga gcccccgatt tagagcttga cggggaaacc ggcgaacgtg |
| 7621 | gcgagaaagg aagggaagaa agcgaaagga gcgggcgcta gggcgctggc aagtgtagcg |
| 7681 | gtcacgctgc gcgtaaccac cacacccgcc gcgctaagcg ccgctacagg gcgcgtccct |
| 7741 | tcgccttcag gctgcgtcga gtactgtact gtgagccaga gttgcccggc gctctccggc |
| 7801 | tgcggtagtt caggcagttc aatcaactgt ttaccttgtg gagcgactcc agaggcactt |
| 7861 | caccgcttgc cagcggctta cgatccagcg ccacgatcca gtgcaggaga tcgttatcgc |
| 7921 | tatacggaac aggtattcgc tggtcacttc gataaggttt gcccggataa acggaactgg |
| 7981 | aaaaactgct gctggtgttt tgcttccgtc agtgctggat cggcgtgcgg tcggcaaaga |
| 8041 | ccagaccgtt ctaacagaac tggcgattgt tcggcgtatc gccaaaatca ccgccgtaag |
| 8101 | ccgaccacgg gttgccgttt tcagcaggat ttaatcagcg actgatccac ccagtcccag |
| 8161 | acgaagccgc cctgtaaacg gggatactga cgaaacgcct gccagtattt agcgaaaccg |
| 8221 | ccaagactgt tacccaagcg tgggcgtatt cgcaaaggat cagcgggcgc gtctctccag |
| 8281 | gtagcgaaag ccttttttga tcgacctttc ggcacagccg ggaagggctg gtcttcaacc |
| 8341 | acgcgcgcgt acaacgggca aataatatcg gtggccgtgg tgtcggctcc gccgccttca |
| 8401 | actgcaccgg gcgggaagga tcgacagatt tgatccagcg atacagcgcg tcgtgattag |
| 8461 | cgccgtggcc tgattcaatt ccccagcgac cagtagatca cactcgggtg attacgattg |
| 8521 | cgctgcacca gtcgcgttac ggttcgctct tcgccggtag ccagcgcgga tcacggtcag |
| 8581 | acgattcgtt ggcacgatcc gtgggtttca atactggctt caaaccacca ctaacaggcc |
| 8641 | gtagcggtcg cacagcgtgt accacagcgg ttggttcgga taatcgaaca gcgcacggcg |
| 8701 | ttaaagttgt tctgcttcaa cagcaggata ttctgcacct tcgtctgctc ttcctaacct |
| 8761 | gaccaagcag aggatctgct cgtgacggtt aatcctcgaa tcagcaacgg cttgccgttc |
| 8821 | agcagcagca gaccaagttc aatccgcacc tcgcggaaac cgacaacgca ggcttctgct |
| 8881 | tcaatcagcg tgccgtcggc ggtgtgcagt tcaaccaccg cacgatagag attcgggatt |
| 8941 | tcggcgctcc acagtttcgg gttttcgacg ttcagacgta gtgtgacgcg atctgcaaac |
| 9001 | caccacgctc aacgataatt tcaccgccga aaggcgcggt gccgctggcg acctgcgttt |
| 9061 | caccctgcca gaaagaaact gttacccgta ggtagtcacg caactcgccg cacactgaac |
| 9121 | ttcagcctcc agtacagcgc ggctgaaatc gtcttaaagc gagtggcaac tggaaatcgc |
| 9181 | tgatttgtgt agtcggttta gcagcaacga gacttcacgg aaaatccgct aatccgccac |
| 9241 | agatcctgat cttccagata actgccgtca ctccaacgca gcaccttcac cgcgaggcgg |
| 9301 | ttttctccgg cgcgtaaaaa tcgctcaggt caaattcaga cggcaaacga ctgtcctggc |
| 9361 | cgtaaccgac ccagcgcccg ttgcaccaca gattgaaacg ccgagtttac gcctcaaaaa |
| 9421 | taattcgcgt ctggccttcc tgtagccagc tttcacaact ataatagtga gcgagtaaca |
| 9481 | acccgtcgga ttctccgtgg gaacaaacgg cggattgacc gtatagggat aggttacgtt |
| 9541 | ggtgtagtag ggcgctccgt aaccgtgcta ctgccagttt gaggggacga cgacagtatc |
| 9601 | ggcctcagga agatcgcact ccagccagct ttccggcacc gcttctggta ctggaaacca |
| 9661 | ggcaaagcgc ctatcgccta tcaggctgca caactgttgg gaagggcgat ctgtgcgggc |
| 9721 | ctcttcgcta ttacgccagc ttgcgaaagg gggtagtgct gcaaggcgat taagttgggt |
| 9781 | aacgccaggg ttttcccagt cacgacgtgg atctgggcca ctccctct |

| pP151? (SEQ ID NO: 508) | |
|---|---|
| (SEQ ID NO: 508) | |
| 1 | ctgcgcgctc gctcgctcac tgaggccgcc cgggcaaagc ccgggcgtcg ggcgaccttt |
| 61 | ggtcgcccgg cctcagtgag cgagcgagcg cgcagagagg gagtggccaa ctccatcact |
| 121 | aggggttcct ggaggggtgg agtcgtgacg tgaattacgt catagggtta gggaggtcgg |
| 181 | ccgctctagg agttaatttt taaaaagcag tcaaaagtcc aagtgccctt gcgagcattt |
| 241 | actctctctg tttgctctgg ttaataatct caggagcaca aacattcctt actagttcta |
| 301 | ggagttaatt tttaaaaagc agtcaaaagt ccaagtgccc ttgcgagcat ttactctctc |
| 361 | tgtttgctct ggttaataat ctcaggagca caaacattcc ttactagttc tagagcggcc |
| 421 | gccagtgtgc tggaattcgg cttttttagg gctggaagct acctttgaca tcatttcctc |
| 481 | tgcgaatgca tgtataattt ctacagaacc tattagaaag gatcacccag cctctgcttt |
| 541 | tgtacaactt tcccttaaaa aactgccaat cccactgctg tttggcccaa tagtgagaac |
| 601 | tttttcctgc tgcctcttgg tgcttttgcc tatggcccct attctgcctg ctgaagacac |
| 661 | tcttgccagc atggacttaa acccctccag ctctgacaat cctctttctc ttttgtttta |
| 721 | catgaagggt ctggcagcca aagcaatcac tcaaagttca aaccttatca ttttttgctt |
| 781 | tgttcctctt ggccttggtt ttgtacatca gctttgaaaa taccatccca gggttaatgc |
| 841 | tggggttaat ttataactga gagtgctcta gttctgcaat acaggacatg ctataaaaat |
| 901 | ggaaagatgt tgctttctga gagatcaggt ggattcttgg gcatttgctt aagcttggta |
| 961 | ccttttttat gggagtgagg cacccgccct gctcccaccg gctcctggcc gtctgcgccc |
| 1021 | tcgtgtcctt ggcaaccgcc atggagtttg ggctgagctg ggtctttctg gtggccctgc |
| 1081 | tgaagggagt ccagtgtgag cagcagggag ccagcagacc agggccccgg gatgcccagg |
| 1141 | cacaccccgg ccgtcccaga gcagtgccca cacagtgcga cgtccccccc aacagccgct |
| 1201 | tcgattgcgc ccctgacaag gccatcaccc aggaacagtg cgaggcccgc ggctgctgct |
| 1261 | acatccctgc aaagcagggg ctgcagggag cccagatggg gcagccctgg tgcttcttcc |
| 1321 | cacccagcta ccccagctac aagctggaga acctgagctc ctctgaaatg ggctacacgg |
| 1381 | ccaccctgac ccgtaccacc cccaccttct tccccaagga catcctgacc ctgcggctgg |
| 1441 | acgtgatgat ggagactgag aaccgcctcc acttcacgat caaagatcca gctaacaggc |
| 1501 | gctacgaggt gcccttggag accccgcgtg tccacagccg ggcaccgtcc ccactctaca |
| 1561 | gcgtggagtt ctccgaggag cccttcgggg tgatcgtgca ccggcagctg gacggccgcg |
| 1621 | tgctgctgaa cacgacggtg gcgcccctgt tctttgcgga ccagttcctt cagctgtcca |
| 1681 | cctcgctgcc ctcgcagtat atcacaggcc tcgccgagca cctcagtccc ctgatgctca |
| 1741 | gcaccagctg gaccaggatc accctgtgga accgggacct tgcgcccacg cccggtgcga |
| 1801 | acctctacgg gtctcaccct ttctacctgg cgctggagga cggcgggtcg gcacacgggg |
| 1861 | tgttcctgct aaacagcaat gccatggatg tggtcctgca gccgagccct gcccttagct |
| 1921 | ggaggtcgac aggtgggatc ctggatgtct acatcttcct gggcccagag cccaagagcg |
| 1981 | tggtgcagca gtacctggac gttgtgggat acccgttcat gccgccatac tggggcctgg |
| 2041 | gcttccacct gtgccgctgg ggctactcct ccaccgctat cacccgccag gtggtggaga |
| 2101 | acatgaccag ggcccacttc cccctggacg tccaatggaa cgacctggac tacatggact |
| 2161 | cccggaggga cttcacgttc aacaaggatg gcttccggga cttcccggcc atggtgcagg |
| 2221 | agctgcacca gggcggccgg cgctacatga tgatcgtgga tcctgccatc agcagctcgg |
| 2281 | gccctgccgg gagctacagg ccctacgacg agggtctgcg gaggggggtt ttcatcacca |
| 2341 | acgagaccgg ccagccgctg attgggaagg tatggcccgg gtccactgcc ttccccgact |
| 2401 | tcaccaaccc cacagccctg gcctggtggg aggacatggt ggctgagttc catgaccagg |
| 2461 | tgcccttcga cggcatgtgg attgacatga acgagccttc caacttcatc agaggctctg |
| 2521 | aggacggctg ccccaacaat gagctggaga acccacccta cgtgcctggg gtggttgggg |
| 2581 | ggaccctcca ggcggccacc atctgtgcct ccagccacca gtttctctcc acacactaca |
| 2641 | acctgcacaa cctctacggc ctgaccgaag ccatcgcctc ccacagggcg ctggtgaagg |
| 2701 | ctcgggggac acgcccattt gtgatctccc gctcgacctt tgctggccac ggccgatacg |
| 2761 | ccggccactg gacgggggac gtgtggagct cctgggagca gctcgcctcc tccgtgccag |
| 2821 | aaatcctgca gtttaacctg ctgggggtgc ctctggtcgg ggccgacgtc tgcggcttcc |
| 2881 | tgggcaacac ctcagaggag ctgtgtgtgc gctggaccca gctgggggcc ttctacccct |
| 2941 | tcatgcggaa ccacaacagc ctgctcagtc tgccccagga gccgtacagc ttcagcgagc |
| 3001 | cggcccagca ggccatgagg aaggccctca ccctgcgcta cgcactcctc ccccacctct |
| 3061 | acacactgtt ccaccaggcc cacgtcgcgg gggagaccgt ggcccggccc ctcttcctgg |
| 3121 | agttccccaa ggactctagc acctggactg tggaccacca gctcctgtgg ggggaggccc |
| 3181 | tgctcatcac cccagtgctc caggccggga aggccgaagt gactggctac ttccccttgg |
| 3241 | gcacatggta cgacctgcag acggtgccaa tagaggccct tggcagcctc ccacccccac |
| 3301 | ctgcagctcc ccgtgagcca gccatccaca gcgaggggca gtgggtgacg ctgccggccc |
| 3361 | ccctggacac catcaacgtc cacctccggg ctgggtacat catccccctg cagggccctg |
| 3421 | gcctcacaac cacagagtcc cgccagcagc ccatggccct ggctgtggcc ctgaccaagg |
| 3481 | gtggagaggc ccgaggggag ctgttctggg acgatggaga gagcctggaa gtgctggagc |
| 3541 | gaggggccta cacacaggtc atcttcctgg ccaggaataa cacgatcgtg aatgagctgg |
| 3601 | tacgtgtgac cagtgaggga gctggcctgc agctgcagaa ggtgactgtc ctgggcgtgg |
| 3661 | ccacggcgcc ccagcaggtc ctctccaacg gtgtccctgt ctccaacttc acctacagcc |
| 3721 | ccgacaccaa ggtcctggac atctgtgtct cgctgttgat gggagagcag tttctcgtca |
| 3781 | gctggtgtta gccgggcgga gtgtgttagt ctctccagag ggaggctggt tccccaggga |
| 3841 | agcagagcct gtgtgcgggc agcagctgtg tgcgggcctg ggggttgtta agtgcaatta |
| 3901 | tttttaataa aaggggcatt tggaaaaaaa aaaaaaaggt agcagtcgac agatgaattc |
| 3961 | tgcagatctg tggcttctag ctgcccgggt ggcatccctg tgacccctcc ccagtgcctc |
| 4021 | tcctggccct ggaagttgcc actccagtgc ccaccagcct tgtcctaata aaattaagtt |
| 4081 | gcatcatttt gtctgactag gtgtccttct ataatattat ggggtggagg ggggtggtat |
| 4141 | ggagcaaggg gcaagttggg aagacaacct gagttgttgg gattccaggc atcgagtaga |
| 4201 | taagtagcat ggcgggttaa tcattaacta caaggaaccc ctagtgatgg agttggccac |
| 4261 | tccctctctg cgcgctcgct cgctcactga ggccgggcga ccaaaggtcg cccgacgccc |
| 4321 | gggctttgcc cgggcggcct cagtgagcga gcgagcgcgc agagagggac agatccgaag |
| 4381 | ccgggcaaat cagcgcctgg cagcagtggc gtctggcgga aaacctcagt gtgacgctcc |
| 4441 | ccgccgcgtc ccacgcttgt tcccggatct gaccaccagc gaaatccgat ttttgcaccg |
| 4501 | agctgggtaa taagcgttgg caatttaacc gccagtcagg ctttctttca cagtgtggat |
| 4561 | tggcgataaa aaacaactgc tgacgccgct gcgcgatcag ttcacccgtt caccgctgga |
| 4621 | taacgacttg gcgtaagtga agcgacccgt aagaccctaa cgcctgggtc gaacgctgga |
| 4681 | aggcggcggg ccaaaccagg ccgaagcagc gttgttgcag ttcacggcag atacacttgc |
| 4741 | tgttgcggtg ctgattacga ccgctcactc gtggcagcaa caggggaaaa ccttatttat |
| 4801 | cagccggaaa acctaccgga ttgttggtag tggtcaatag gcgattaccg ttgtgttgaa |
| 4861 | gtggcgagcg atacaccgct tccggcgcgg attggcctga actgccaact ggcgcaggta |
| 4921 | gcagagcggg taaactggct cggattaggg ccgcaagaaa actatcccga ccgccttact |
| 4981 | gccgcctgtt ttgaccgctg ggatctgcca agtcagacag tatagcccgt acgtcttccc |
| 5041 | gagcgaaaac ggtctgcgct gcgggacgcg cgaattgaat ttggcccaca ccagtggcgc |
| 5101 | ggcgacttcc agttcaatat cagccgctac agtgaacagc aactgttgga aaccagcctt |
| 5161 | cgccaactgc tgcacgcgga agaaggcact ggctgaatat cgacggtttc cagttgggga |
| 5221 | ttggtggcga cgactcctgg agcccgtcag tatcggcgga cttccaactg agcgccggtc |
| 5281 | gctaccttac cagttggtct ggtgtcaaaa agcgtccgct tgagtctagc gatcgcgcgc |
| 5341 | agatctgtca tgtgagcaaa aggccagcaa aaggccagga accgtaaaaa ggccgcgttg |
| 5401 | ctggcgtttt tccataggct ccgcccccct gacgagcatc acaaaaatcg acgctcaagt |
| 5461 | cagaggtggc gaaacccgac aggactataa agataccagg cgtttccccc tggaagctcc |
| 5521 | ctcgtgcgct ctcctgttcc gaccctgccg cttaccggat acctgtccgc ctttctccct |
| 5581 | tcgggaagcg tggcgctttc tcatagctca cgctgtaggt atctcagttc ggtgtaggtc |
| 5641 | gttcgctcca agctgggctg tgtgcacgaa ccccccgttc agcccgaccg ctgcgcctta |
| 5701 | tccggtaact atcgtcttga gtccaacccg gtaagacacg acttatcgcc actggcagca |
| 5761 | gccactggta acaggattag cagagcgagg tatgtaggcg gtgctacaga gttcttgaag |
| 5821 | tggtggccta actacggcta cactagaaga acagtatttg gtatctgcgc tctgctgaag |
| 5881 | ccagttacct tcggaaaaag agttggtagc tcttgatccg gcaaacaaac caccgctggt |
| 5941 | agcggtggtt tttttgtttg caagcagcag attacgcgca gaaaaaaagg atctcaagaa |
| 6001 | gatcctttga tcttttctac ggggtctgac gctcagtgga acgaaaactc acgttaaggg |
| 6061 | attttggtca tgagattatc aaaaaggatc ttcacctaga tccttttcac gtagaaagcc |
| 6121 | agtccgcaga aacggtgctg accccggatg aatgtcagct actgggctat ctggacaagg |
| 6181 | gaaaacgcaa gcgcaaagag aaagcaggta gcttgcagtg ggcttacatg gcgatagcta |
| 6241 | gactgggcgg ttttatggac agcaagcgaa ccggaattgc cagctggggc gccctctggt |
| 6301 | aaggttggga agccctgcaa agtaaactgg atggctttct tgccgccaag gatctgatgg |
| 6361 | cgcaggggat caagatctga tcaagagaca ggatgaggat cgtttcgcat gattgaacaa |
| 6421 | gatggattgc acgcaggttc tccggccgct tgggtggaga ggctattcgg ctatgactgg |
| 6481 | gcacaacaga caatcggctg ctctgatgcc gccgtgttcc ggctgtcagc gcaggggcgc |
| 6541 | ccggttcttt ttgtcaagac cgacctgtcc ggtgccctga atgaactgca agacgaggca |
| 6601 | gcgcggctat cgtggctggc cacgacgggc gttccttgcg cagctgtgct cgacgttgtc |
| 6661 | actgaagcgg gaagggactg gctgctattg ggcgaagtgc cggggcagga tctcctgtca |
| 6721 | tctcaccttg ctcctgccga gaaagtatcc atcatggctg atgcaatgcg gcggctgcat |
| 6781 | acgcttgatc cggctacctg cccattcgac caccaagcga aacatcgcat cgagcgagca |
| 6841 | cgtactcgga tggaagccgg tcttgtcgat caggatgatc tggacgaaga gcatcagggg |
| 6901 | ctcgcgccag ccgaactgtt cgccaggctc aaggcgagca tgcccgacgg cgaggatctc |
| 6961 | gtcgtgaccc atggcgatgc ctgcttgccg aatatcatgg tggaaaatgg ccgcttttct |
| 7021 | ggattcatcg actgtggccg gctgggtgtg gcggaccgct atcaggacat agcgttggct |
| 7081 | acccgtgata ttgctgaaga gcttggcggc gaatgggctg accgcttcct cgtgctttac |
| 7141 | ggtatcgccg ctcccgattc gcagcgcatc gccttctatc gccttcttga cgagttcttc |
| 7201 | tgaatttaaa gcccaatacg caaaccgcct ctccccgcgc gttggccgaa gcggttcaat |
| 7261 | attttgttaa aattcgcgtt aaatttttgt taaatcagct attttttaac caataggccg |
| 7321 | aaatcggcaa aatcccttgt aaatcaaaag aatagaccga gatagggttg agtgttgttc |
| 7381 | cagtttggaa caagagtcca ctattaaaga acgtggactc caacgtcaaa gggcgaaaaa |
| 7441 | ccgtctatca gggcgttggc ccactacgtg aaccttcacc ctaatcaagt tttttggggt |
| 7501 | cgaggtgccg taaagcacta aatcggaacc ctaaagggag cccccgattt agagcttgac |
| 7561 | ggggaaaccg gcgaacgtgg cgagaaagga agggaagaaa gcgaaaggag cgggcgctag |
| 7621 | ggcgctggca agtgtagcgg tcacgctgcg cgtaaccacc acacccgccg cgctaagcgc |
| 7681 | cgctacaggg cgcgtccctt cgccttcagg ctgcgtcgag tactgtactg tgagccagag |
| 7741 | ttgcccggcg ctctccggct gcggtagttc aggcagttca atcaactgtt taccttgtgg |
| 7801 | agcgactcca gaggcacttc accgcttgcc agcggcttac gatccagcgc cacgatccag |
| 7861 | tgcaggagat cgttatcgct atacggaaca ggtattcgct ggtcacttcg ataaggtttg |
| 7921 | cccggataaa cggaactgga aaaactgctg ctggtgtttt gcttccgtca gtgctggatc |
| 7981 | ggcgtgcggt cggcaaagac cagaccgttc taacagaact ggcgattgtt cggcgtatcg |
| 8041 | ccaaaatcac cgccgtaagc cgaccacggg ttgccgtttt cagcaggatt taatcagcga |
| 8101 | ctgatccacc cagtcccaga cgaagccgcc ctgtaaacgg ggatactgac gaaacgcctg |
| 8161 | ccagtattta gcgaaaccgc caagactgtt acccaagcgt gggcgtattc gcaaaggatc |
| 8221 | agcgggcgcg tctctccagg tagcgaaagc cttttttgat cgacctttcg gcacagccgg |
| 8281 | gaagggctgg tcttcaacca cgcgcgcgta caacgggcaa ataatatcgg tggccgtggt |
| 8341 | gtcggctccg ccgccttcaa ctgcaccggg cgggaaggat cgacagattt gatccagcga |
| 8401 | tacagcgcgt cgtgattagc gccgtggcct gattcaattc cccagcgacc agtagatcac |
| 8461 | actcgggtga ttacgattgc gctgcaccag tcgcgttacg gttcgctctt cgccggtagc |
| 8521 | cagcgcggat cacggtcaga cgattcgttg gcacgatccg tgggtttcaa tactggcttc |
| 8581 | aaaccaccac taacaggccg tagcggtcgc acagcgtgta ccacagcggt tggttcggat |
| 8641 | aatcgaacag cgcacggcgt taaagttgtt ctgcttcaac agcaggatat tctgcacctt |
| 8701 | cgtctgctct tcctaacctg accaagcaga ggatctgctc gtgacggtta atcctcgaat |
| 8761 | cagcaacggc ttgccgttca gcagcagcag accaagttca atccgcacct cgcggaaacc |
| 8821 | gacaacgcag gcttctgctt caatcagcgt gccgtcggcg gtgtgcagtt caaccaccgc |
| 8881 | acgatagaga ttcgggattt cggcgctcca cagtttcggg ttttcgacgt tcagacgtag |
| 8941 | tgtgacgcga tctgcaaacc accacgctca acgataattt caccgccgaa aggcgcggtg |
| 9001 | ccgctggcga cctgcgtttc accctgccag aaagaaactg ttacccgtag gtagtcacgc |
| 9061 | aactcgccgc acactgaact tcagcctcca gtacagcgcg gctgaaatcg tcttaaagcg |
| 9121 | agtggcaact ggaaatcgct gatttgtgta gtcggtttag cagcaacgag acttcacgga |
| 9181 | aaatccgcta atccgccaca gatcctgatc ttccagataa ctgccgtcac tccaacgcag |
| 9241 | caccttcacc gcgaggcggt tttctccggc gcgtaaaaat cgctcaggtc aaattcagac |
| 9301 | ggcaaacgac tgtcctggcc gtaaccgacc cagcgcccgt tgcaccacag attgaaacgc |
| 9361 | cgagtttacg cctcaaaaat aattcgcgtc tggccttcct gtagccagct ttcacaacta |
| 9421 | taatagtgag cgagtaacaa cccgtcggat tctccgtggg aacaaacggc ggattgaccg |
| 9481 | tatagggata ggttacgttg gtgtagtagg gcgctccgta accgtgctac tgccagtttg |
| 9541 | aggggacgac gacagtatcg gcctcaggaa gatcgcactc cagccagctt tccggcaccg |
| 9601 | cttctggtac tggaaaccag gcaaagcgcc tatcgcctat caggctgcac aactgttggg |
| 9661 | aagggcgatc tgtgcgggcc tcttcgctat tacgccagct tgcgaaaggg ggtagtgctg |
| 9721 | caaggcgatt aagttgggta acgccagggt tttcccagtc acgacgtgga tctgggccac |
| 9781 | tccctct |

| pP152 (SEQ ID NO: 509) | |
|---|---|
| (SEQ ID NO: 509) | |
| 1 | ctgcgcgctc gctcgctcac tgaggccgcc cgggcaaagc ccgggcgtcg ggcgaccttt |
| 61 | ggtcgcccgg cctcagtgag cgagcgagcg cgcagagagg gagtggccaa ctccatcact |
| 121 | aggggttcct ggaggggtgg agtcgtgacg tgaattacgt catagggtta gggaggtcgg |
| 181 | ccgctctagg agttaatttt taaaaagcag tcaaaagtcc aagtgccctt gcgagcattt |
| 241 | actctctctg tttgctctgg ttaataatct caggagcaca aacattcctt actagttcta |
| 301 | ggagttaatt tttaaaaagc agtcaaaagt ccaagtgccc ttgcgagcat ttactctctc |
| 361 | tgtttgctct ggttaataat ctcaggagca caaacattcc ttactagttc tagagcggcc |
| 421 | gccagtgtgc tggaattcgg cttttttagg gctggaagct acctttgaca tcatttcctc |
| 481 | tgcgaatgca tgtataattt ctacagaacc tattagaaag gatcacccag cctctgcttt |
| 541 | tgtacaactt tcccttaaaa aactgccaat cccactgctg tttggcccaa tagtgagaac |
| 601 | tttttcctgc tgcctcttgg tgcttttgcc tatggcccct attctgcctg ctgaagacac |
| 661 | tcttgccagc atggacttaa acccctccag ctctgacaat cctctttctc ttttgtttta |
| 721 | catgaagggt ctggcagcca aagcaatcac tcaaagttca aaccttatca ttttttgctt |
| 781 | tgttcctctt ggccttggtt ttgtacatca gctttgaaaa taccatccca gggttaatgc |
| 841 | tggggttaat ttataactga gagtgctcta gttctgcaat acaggacatg ctataaaaat |
| 901 | ggaaagatgt tgctttctga gagatcagct tacatgtggt accgagctcg gatccactag |
| 961 | taacggccgc cagtgtgctg gaattcggct ttccaggcca tcttttttta tgggggtgag |
| 1021 | gcaccctccc tgttctcaca ggctgctggc tgtgtgtgct ctggtgtctc tggccactgc |
| 1081 | catggagttt gggctgagct gggtctttct ggtggccctg ctgaagggag tccagtgtga |
| 1141 | gcagcaggga gccagcagac cagggccccg ggatgcccag gcacaccccg gccgtcccag |
| 1201 | agcagtgccc acacagtgcg acgtcccccc caacagccgc ttcgattgcg cccctgacaa |
| 1261 | ggccatcacc caggaacagt gcgaggcccg cggctgctgc tacatccctg caaagcaggg |
| 1321 | gctgcaggga gcccagatgg ggcagccctg gtgcttcttc ccacccagct accccagcta |
| 1381 | caagctggag aacctgagct cctctgaaat gggctacacg gccaccctga cccgtaccac |
| 1441 | ccccaccttc ttccccaagg acatcctgac cctgcggctg gacgtgatga tggagactga |
| 1501 | gaaccgcctc cacttcacga tcaaagatcc agctaacagg cgctacgagg tgcccttgga |
| 1561 | gaccccgcgt gtccacagcc gggcaccgtc cccactctac agcgtggagt tctccgagga |
| 1621 | gcccttcggg gtgatcgtgc accggcagct ggacggccgc gtgctgctga acacgacggt |
| 1681 | ggcgcccctg ttctttgcgg accagttcct tcagctgtcc acctcgctgc cctcgcagta |
| 1741 | tatcacaggc ctcgccgagc acctcagtcc cctgatgctc agcaccagct ggaccaggat |
| 1801 | caccctgtgg aaccgggacc ttgcgcccac gcccggtgcg aacctctacg ggtctcaccc |
| 1861 | tttctacctg gcgctggagg acggcgggtc ggcacacggg gtgttcctgc taaacagcaa |
| 1921 | tgccatggat gtggtcctgc agccgagccc tgcccttagc tggaggtcga caggtgggat |
| 1981 | cctggatgtc tacatcttcc tgggcccaga gcccaagagc gtggtgcagc agtacctgga |
| 2041 | cgttgtggga tacccgttca tgccgccata ctggggcctg ggcttccacc tgtgccgctg |
| 2101 | gggctactcc tccaccgcta tcacccgcca ggtggtggag aacatgacca gggcccactt |
| 2161 | ccccctggac gtccaatgga acgacctgga ctacatggac tcccggaggg acttcacgtt |
| 2221 | caacaaggat ggcttccggg acttcccggc catggtgcag gagctgcacc agggcggccg |
| 2281 | gcgctacatg atgatcgtgg atcctgccat cagcagctcg ggccctgccg ggagctacag |
| 2341 | gccctacgac gagggtctgc ggaggggggt tttcatcacc aacgagaccg gccagccgct |
| 2401 | gattgggaag gtatggcccg ggtccactgc cttccccgac ttcaccaacc ccacagccct |
| 2461 | ggcctggtgg gaggacatgg tggctgagtt ccatgaccag gtgcccttcg acggcatgtg |
| 2521 | gattgacatg aacgagcctt ccaacttcat cagaggctct gaggacggct gccccaacaa |
| 2581 | tgagctggag aacccaccct acgtgcctgg ggtggttggg gggaccctcc aggcggccac |
| 2641 | catctgtgcc tccagccacc agtttctctc cacacactac aacctgcaca acctctacgg |
| 2701 | cctgaccgaa gccatcgcct cccacagggc gctggtgaag gctcggggga cacgcccatt |
| 2761 | tgtgatctcc cgctcgacct ttgctggcca cggccgatac gccggccact ggacggggga |
| 2821 | cgtgtggagc tcctgggagc agctcgcctc ctccgtgcca gaaatcctgc agtttaacct |
| 2881 | gctgggggtg cctctggtcg gggccgacgt ctgcggcttc ctgggcaaca cctcagagga |
| 2941 | gctgtgtgtg cgctggaccc agctgggggc cttctacccc ttcatgcgga accacaacag |
| 3001 | cctgctcagt ctgccccagg agccgtacag cttcagcgag ccggcccagc aggccatgag |
| 3061 | gaaggccctc accctgcgct acgcactcct cccccacctc tacacactgt tccaccaggc |
| 3121 | ccacgtcgcg ggggagaccg tggcccggcc cctcttcctg gagttcccca aggactctag |
| 3181 | cacctggact gtggaccacc agctcctgtg gggggaggcc ctgctcatca ccccagtgct |
| 3241 | ccaggccggg aaggccgaag tgactggcta cttccccttg ggcacatggt acgacctgca |
| 3301 | gacggtgcca atagaggccc ttggcagcct cccaccccca cctgcagctc cccgtgagcc |
| 3361 | agccatccac agcgaggggc agtgggtgac gctgccggcc cccctggaca ccatcaacgt |
| 3421 | ccacctccgg gctgggtaca tcatccccct gcagggccct ggcctcacaa ccacagagtc |
| 3481 | ccgccagcag cccatggccc tggctgtggc cctgaccaag ggtggagagg cccgagggga |
| 3541 | gctgttctgg gacgatggag agagcctgga agtgctggag cgaggggcct acacacaggt |
| 3601 | catcttcctg gccaggaata acacgatcgt gaatgagctg gtacgtgtga ccagtgaggg |
| 3661 | agctggcctg cagctgcaga aggtgactgt cctgggcgtg gccacggcgc cccagcaggt |
| 3721 | cctctccaac ggtgtccctg tctccaactt cacctacagc cccgacacca aggtcctgga |
| 3781 | catctgtgtc tcgctgttga tgggagagca gtttctcgtc agctggtgtt agccgggcgg |
| 3841 | agtgtgttag tctctccaga gggaggctgg ttccccaggg aagcagagcc tgtgtgcggg |
| 3901 | cagcagctgt gtgcgggcct gggggttgtt aagtgcaatt atttttaata aaaggggcat |
| 3961 | ttggaaaaaa aaaaaaaagg tagcagtcga cagatgaatt ctgcagatct gtggcttcta |
| 4021 | gctgcccggg tggcatccct gtgacccctc cccagtgcct ctcctggccc tggaagttgc |
| 4081 | cactccagtg cccaccagcc ttgtcctaat aaaattaagt tgcatcattt tgtctgacta |
| 4141 | ggtgtccttc tataatatta tggggtggag gggggtggta tggagcaagg ggcaagttgg |
| 4201 | gaagacaacc tgagttgttg ggattccagg catcgagtag ataagtagca tggcgggtta |
| 4261 | atcattaact acaaggaacc cctagtgatg gagttggcca ctccctctct gcgcgctcgc |
| 4321 | tcgctcactg aggccgggcg accaaaggtc gcccgacgcc cgggctttgc ccgggcggcc |
| 4381 | tcagtgagcg agcgagcgcg cagagaggga cagatccgaa gccgggcaaa tcagcgcctg |
| 4441 | gcagcagtgg cgtctggcgg aaaacctcag tgtgacgctc cccgccgcgt cccacgcttg |
| 4501 | ttcccggatc tgaccaccag cgaaatccga tttttgcacc gagctgggta ataagcgttg |
| 4561 | gcaatttaac cgccagtcag gctttctttc acagtgtgga ttggcgataa aaaacaactg |
| 4621 | ctgacgccgc tgcgcgatca gttcacccgt tcaccgctgg ataacgactt ggcgtaagtg |
| 4681 | aagcgacccg taagacccta acgcctgggt cgaacgctgg aaggcggcgg gccaaaccag |
| 4741 | gccgaagcag cgttgttgca gttcacggca gatacacttg ctgttgcggt gctgattacg |
| 4801 | accgctcact cgtggcagca acaggggaaa accttattta tcagccggaa aacctaccgg |
| 4861 | attgttggta gtggtcaata ggcgattacc gttgtgttga agtggcgagc gatacaccgc |
| 4921 | ttccggcgcg gattggcctg aactgccaac tggcgcaggt agcagagcgg gtaaactggc |
| 4981 | tcggattagg gccgcaagaa aactatcccg accgccttac tgccgcctgt tttgaccgct |
| 5041 | gggatctgcc aagtcagaca gtatagcccg tacgtcttcc cgagcgaaaa cggtctgcgc |
| 5101 | tgcgggacgc gcgaattgaa tttggcccac accagtggcg cggcgacttc cagttcaata |
| 5161 | tcagccgcta cagtgaacag caactgttgg aaaccagcct tcgccaactg ctgcacgcgg |
| 5221 | aagaaggcac tggctgaata tcgacggttt ccagttgggg attggtggcg acgactcctg |
| 5281 | gagcccgtca gtatcggcgg acttccaact gagcgccggt cgctacctta ccagttggtc |
| 5341 | tggtgtcaaa aagcgtccgc ttgagtctag cgatcgcgcg cagatctgtc atgtgagcaa |
| 5401 | aaggccagca aaaggccagg aaccgtaaaa aggccgcgtt gctggcgttt ttccataggc |
| 5461 | tccgcccccc tgacgagcat cacaaaaatc gacgctcaag tcagaggtgg cgaaacccga |
| 5521 | caggactata aagataccag gcgtttcccc ctggaagctc cctcgtgcgc tctcctgttc |
| 5581 | cgaccctgcc gcttaccgga tacctgtccg cctttctccc ttcgggaagc gtggcgcttt |
| 5641 | ctcatagctc acgctgtagg tatctcagtt cggtgtaggt cgttcgctcc aagctgggct |
| 5701 | gtgtgcacga accccccgtt cagcccgacc gctgcgcctt atccggtaac tatcgtcttg |
| 5761 | agtccaaccc ggtaagacac gacttatcgc cactggcagc agccactggt aacaggatta |
| 5821 | gcagagcgag gtatgtaggc ggtgctacag agttcttgaa gtggtggcct aactacggct |
| 5881 | acactagaag aacagtattt ggtatctgcg ctctgctgaa gccagttacc ttcggaaaaa |
| 5941 | gagttggtag ctcttgatcc ggcaaacaaa ccaccgctgg tagcggtggt ttttttgttt |
| 6001 | gcaagcagca gattacgcgc agaaaaaaag gatctcaaga agatcctttg atcttttcta |
| 6061 | cggggtctga cgctcagtgg aacgaaaact cacgttaagg gattttggtc atgagattat |
| 6121 | caaaaaggat cttcacctag atccttttca cgtagaaagc cagtccgcag aaacggtgct |
| 6181 | gaccccggat gaatgtcagc tactgggcta tctggacaag ggaaaacgca agcgcaaaga |
| 6241 | gaaagcaggt agcttgcagt gggcttacat ggcgatagct agactgggcg gttttatgga |
| 6301 | cagcaagcga accggaattg ccagctgggg cgccctctgg taaggttggg aagccctgca |
| 6361 | aagtaaactg gatggctttc ttgccgccaa ggatctgatg gcgcagggga tcaagatctg |
| 6421 | atcaagagac aggatgagga tcgtttcgca tgattgaaca agatggattg cacgcaggtt |
| 6481 | ctccggccgc ttgggtggag aggctattcg gctatgactg ggcacaacag acaatcggct |
| 6541 | gctctgatgc cgccgtgttc cggctgtcag cgcaggggcg cccggttctt tttgtcaaga |
| 6601 | ccgacctgtc cggtgccctg aatgaactgc aagacgaggc agcgcggcta tcgtggctgg |
| 6661 | ccacgacggg cgttccttgc gcagctgtgc tcgacgttgt cactgaagcg ggaagggact |
| 6721 | ggctgctatt gggcgaagtg ccggggcagg atctcctgtc atctcacctt gctcctgccg |
| 6781 | agaaagtatc catcatggct gatgcaatgc ggcggctgca tacgcttgat ccggctacct |
| 6841 | gcccattcga ccaccaagcg aaacatcgca tcgagcgagc acgtactcgg atggaagccg |
| 6901 | gtcttgtcga tcaggatgat ctggacgaag agcatcaggg gctcgcgcca gccgaactgt |
| 6961 | tcgccaggct caaggcgagc atgcccgacg gcgaggatct cgtcgtgacc catggcgatg |
| 7021 | cctgcttgcc gaatatcatg gtggaaaatg gccgcttttc tggattcatc gactgtggcc |
| 7081 | ggctgggtgt ggcggaccgc tatcaggaca tagcgttggc tacccgtgat attgctgaag |
| 7141 | agcttggcgg cgaatgggct gaccgcttcc tcgtgcttta cggtatcgcc gctcccgatt |
| 7201 | cgcagcgcat cgccttctat cgccttcttg acgagttctt ctgaatttaa agcccaatac |
| 7261 | gcaaaccgcc tctccccgcg cgttggccga agcggttcaa tattttgtta aaattcgcgt |
| 7321 | taaatttttg ttaaatcagc tattttttaa ccaataggcc gaaatcggca aaatcccttg |
| 7381 | taaatcaaaa gaatagaccg agatagggtt gagtgttgtt ccagtttgga acaagagtcc |
| 7441 | actattaaag aacgtggact ccaacgtcaa agggcgaaaa accgtctatc agggcgttgg |
| 7501 | cccactacgt gaaccttcac cctaatcaag ttttttgggg tcgaggtgcc gtaaagcact |
| 7561 | aaatcggaac cctaaaggga gcccccgatt tagagcttga cggggaaacc ggcgaacgtg |
| 7621 | gcgagaaagg aagggaagaa agcgaaagga gcgggcgcta gggcgctggc aagtgtagcg |
| 7681 | gtcacgctgc gcgtaaccac cacacccgcc gcgctaagcg ccgctacagg gcgcgtccct |
| 7741 | tcgccttcag gctgcgtcga gtactgtact gtgagccaga gttgcccggc gctctccggc |
| 7801 | tgcggtagtt caggcagttc aatcaactgt ttaccttgtg gagcgactcc agaggcactt |
| 7861 | caccgcttgc cagcggctta cgatccagcg ccacgatcca gtgcaggaga tcgttatcgc |
| 7921 | tatacggaac aggtattcgc tggtcacttc gataaggttt gcccggataa acggaactgg |
| 7981 | aaaaactgct gctggtgttt tgcttccgtc agtgctggat cggcgtgcgg tcggcaaaga |
| 8041 | ccagaccgtt ctaacagaac tggcgattgt tcggcgtatc gccaaaatca ccgccgtaag |
| 8101 | ccgaccacgg gttgccgttt tcagcaggat ttaatcagcg actgatccac ccagtcccag |
| 8161 | acgaagccgc cctgtaaacg gggatactga cgaaacgcct gccagtattt agcgaaaccg |
| 8221 | ccaagactgt tacccaagcg tgggcgtatt cgcaaaggat cagcgggcgc gtctctccag |
| 8281 | gtagcgaaag ccttttttga tcgacctttc ggcacagccg ggaagggctg gtcttcaacc |
| 8341 | acgcgcgcgt acaacgggca aataatatcg gtggccgtgg tgtcggctcc gccgccttca |
| 8401 | actgcaccgg gcgggaagga tcgacagatt tgatccagcg atacagcgcg tcgtgattag |
| 8461 | cgccgtggcc tgattcaatt ccccagcgac cagtagatca cactcgggtg attacgattg |
| 8521 | cgctgcacca gtcgcgttac ggttcgctct tcgccggtag ccagcgcgga tcacggtcag |
| 8581 | acgattcgtt ggcacgatcc gtgggtttca atactggctt caaaccacca ctaacaggcc |
| 8641 | gtagcggtcg cacagcgtgt accacagcgg ttggttcgga taatcgaaca gcgcacggcg |
| 8701 | ttaaagttgt tctgcttcaa cagcaggata ttctgcacct tcgtctgctc ttcctaacct |
| 8761 | gaccaagcag aggatctgct cgtgacggtt aatcctcgaa tcagcaacgg cttgccgttc |
| 8821 | agcagcagca gaccaagttc aatccgcacc tcgcggaaac cgacaacgca ggcttctgct |
| 8881 | tcaatcagcg tgccgtcggc ggtgtgcagt tcaaccaccg cacgatagag attcgggatt |
| 8941 | tcggcgctcc acagtttcgg gttttcgacg ttcagacgta gtgtgacgcg atctgcaaac |
| 9001 | caccacgctc aacgataatt tcaccgccga aaggcgcggt gccgctggcg acctgcgttt |
| 9061 | caccctgcca gaaagaaact gttacccgta ggtagtcacg caactcgccg cacactgaac |
| 9121 | ttcagcctcc agtacagcgc ggctgaaatc gtcttaaagc gagtggcaac tggaaatcgc |
| 9181 | tgatttgtgt agtcggttta gcagcaacga gacttcacgg aaaatccgct aatccgccac |
| 9241 | agatcctgat cttccagata actgccgtca ctccaacgca gcaccttcac cgcgaggcgg |
| 9301 | ttttctccgg cgcgtaaaaa tcgctcaggt caaattcaga cggcaaacga ctgtcctggc |
| 9361 | cgtaaccgac ccagcgcccg ttgcaccaca gattgaaacg ccgagtttac gcctcaaaaa |
| 9421 | taattcgcgt ctggccttcc tgtagccagc tttcacaact ataatagtga gcgagtaaca |
| 9481 | acccgtcgga ttctccgtgg gaacaaacgg cggattgacc gtatagggat aggttacgtt |
| 9541 | ggtgtagtag ggcgctccgt aaccgtgcta ctgccagttt gaggggacga cgacagtatc |
| 9601 | ggcctcagga agatcgcact ccagccagct ttccggcacc gcttctggta ctggaaacca |
| 9661 | ggcaaagcgc ctatcgccta tcaggctgca caactgttgg gaagggcgat ctgtgcgggc |
| 9721 | ctcttcgcta ttacgccagc ttgcgaaagg gggtagtgct gcaaggcgat taagttgggt |
| 9781 | aacgccaggg ttttcccagt cacgacgtgg atctgggcca ctccctct |

| pP153 (SEQ ID NO: 510) | |
|---|---|
| (SEQ ID NO: 510) | |
| 1 | ctgcgcgctc gctcgctcac tgaggccgcc cgggcaaagc ccgggcgtcg ggcgaccttt |
| 61 | ggtcgcccgg cctcagtgag cgagcgagcg cgcagagagg gagtggccaa ctccatcact |
| 121 | aggggttcct ggaggggtgg agtcgtgacg tgaattacgt catagggtta gggaggtcgg |
| 181 | ccgctctagg agttaatttt taaaaagcag tcaaaagtcc aagtgccctt gcgagcattt |
| 241 | actctctctg tttgctctgg ttaataatct caggagcaca aacattcctt actagttcta |
| 301 | ggagttaatt tttaaaaagc agtcaaaagt ccaagtgccc ttgcgagcat ttactctctc |
| 361 | tgtttgctct ggttaataat ctcaggagca caaacattcc ttactagttc tagagcggcc |
| 421 | gccagtgtgc tggaattcgg cttttttagg gctggaagct acctttgaca tcatttcctc |
| 481 | tgcgaatgca tgtataattt ctacagaacc tattagaaag gatcacccag cctctgcttt |
| 541 | tgtacaactt tcccttaaaa aactgccaat cccactgctg tttggcccaa tagtgagaac |
| 601 | tttttcctgc tgcctcttgg tgcttttgcc tatggcccct attctgcctg ctgaagacac |
| 661 | tcttgccagc atggacttaa acccctccag ctctgacaat cctctttctc ttttgtttta |
| 721 | catgaagggt ctggcagcca aagcaatcac tcaaagttca aaccttatca ttttttgctt |
| 781 | tgttcctctt ggccttggtt ttgtacatca gctttgaaaa taccatccca gggttaatgc |
| 841 | tggggttaat ttataactga gagtgctcta gttctgcaat acaggacatg ctataaaaat |
| 901 | ggaaagatgt tgctttctga gagatcaggt ggattcttgg gcatttgctt aagcttggta |
| 961 | ccttttttat gggggtgagg caccctccct gttctcacag gctgctggct gtgtgtgctc |
| 1021 | tggtgtctct ggccactgcc atggagtttg ggctgagctg ggtctttctg gtggccctgc |
| 1081 | tgaagggagt ccagtgtgag cagcagggag ccagcagacc agggccccgg gatgcccagg |
| 1141 | cacaccccgg ccgtcccaga gcagtgccca cacagtgcga cgtccccccc aacagccgct |
| 1201 | tcgattgcgc ccctgacaag gccatcaccc aggaacagtg cgaggcccgc ggctgctgct |
| 1261 | acatccctgc aaagcagggg ctgcagggag cccagatggg gcagccctgg tgcttcttcc |
| 1321 | cacccagcta ccccagctac aagctggaga acctgagctc ctctgaaatg ggctacacgg |
| 1381 | ccaccctgac ccgtaccacc cccaccttct tccccaagga catcctgacc ctgcggctgg |
| 1441 | acgtgatgat ggagactgag aaccgcctcc acttcacgat caaagatcca gctaacaggc |
| 1501 | gctacgaggt gcccttggag accccgcgtg tccacagccg ggcaccgtcc ccactctaca |
| 1561 | gcgtggagtt ctccgaggag cccttcgggg tgatcgtgca ccggcagctg gacggccgcg |
| 1621 | tgctgctgaa cacgacggtg gcgcccctgt tctttgcgga ccagttcctt cagctgtcca |
| 1681 | cctcgctgcc ctcgcagtat atcacaggcc tcgccgagca cctcagtccc ctgatgctca |
| 1741 | gcaccagctg gaccaggatc accctgtgga accgggacct tgcgcccacg cccggtgcga |
| 1801 | acctctacgg gtctcaccct ttctacctgg cgctggagga cggcgggtcg gcacacgggg |
| 1861 | tgttcctgct aaacagcaat gccatggatg tggtcctgca gccgagccct gcccttagct |
| 1921 | ggaggtcgac aggtgggatc ctggatgtct acatcttcct gggcccagag cccaagagcg |
| 1981 | tggtgcagca gtacctggac gttgtgggat acccgttcat gccgccatac tggggcctgg |
| 2041 | gcttccacct gtgccgctgg ggctactcct ccaccgctat cacccgccag gtggtggaga |
| 2101 | acatgaccag ggcccacttc cccctggacg tccaatggaa cgacctggac tacatggact |
| 2161 | cccggaggga cttcacgttc aacaaggatg gcttccggga cttcccggcc atggtgcagg |
| 2221 | agctgcacca gggcggccgg cgctacatga tgatcgtgga tcctgccatc agcagctcgg |
| 2281 | gccctgccgg gagctacagg ccctacgacg agggtctgcg gaggggggtt ttcatcacca |
| 2341 | acgagaccgg ccagccgctg attgggaagg tatggcccgg gtccactgcc ttccccgact |
| 2401 | tcaccaaccc cacagccctg gcctggtggg aggacatggt ggctgagttc catgaccagg |
| 2461 | tgcccttcga cggcatgtgg attgacatga acgagccttc caacttcatc agaggctctg |
| 2521 | aggacggctg ccccaacaat gagctggaga acccacccta cgtgcctggg gtggttgggg |
| 2581 | ggaccctcca ggcggccacc atctgtgcct ccagccacca gtttctctcc acacactaca |
| 2641 | acctgcacaa cctctacggc ctgaccgaag ccatcgcctc ccacagggcg ctggtgaagg |
| 2701 | ctcgggggac acgcccattt gtgatctccc gctcgacctt tgctggccac ggccgatacg |
| 2761 | ccggccactg gacgggggac gtgtggagct cctgggagca gctcgcctcc tccgtgccag |
| 2821 | aaatcctgca gtttaacctg ctgggggtgc ctctggtcgg ggccgacgtc tgcggcttcc |
| 2881 | tgggcaacac ctcagaggag ctgtgtgtgc gctggaccca gctgggggcc ttctacccct |
| 2941 | tcatgcggaa ccacaacagc ctgctcagtc tgccccagga gccgtacagc ttcagcgagc |
| 3001 | cggcccagca ggccatgagg aaggccctca ccctgcgcta cgcactcctc ccccacctct |
| 3061 | acacactgtt ccaccaggcc cacgtcgcgg gggagaccgt ggcccggccc ctcttcctgg |
| 3121 | agttccccaa ggactctagc acctggactg tggaccacca gctcctgtgg ggggaggccc |
| 3181 | tgctcatcac cccagtgctc caggccggga aggccgaagt gactggctac ttccccttgg |
| 3241 | gcacatggta cgacctgcag acggtgccaa tagaggccct tggcagcctc ccacccccac |
| 3301 | ctgcagctcc ccgtgagcca gccatccaca gcgaggggca gtgggtgacg ctgccggccc |
| 3361 | ccctggacac catcaacgtc cacctccggg ctgggtacat catccccctg cagggccctg |
| 3421 | gcctcacaac cacagagtcc cgccagcagc ccatggccct ggctgtggcc ctgaccaagg |
| 3481 | gtggagaggc ccgaggggag ctgttctggg acgatggaga gagcctggaa gtgctggagc |
| 3541 | gaggggccta cacacaggtc atcttcctgg ccaggaataa cacgatcgtg aatgagctgg |
| 3601 | tacgtgtgac cagtgaggga gctggcctgc agctgcagaa ggtgactgtc ctgggcgtgg |
| 3661 | ccacggcgcc ccagcaggtc ctctccaacg gtgtccctgt ctccaacttc acctacagcc |
| 3721 | ccgacaccaa ggtcctggac atctgtgtct cgctgttgat gggagagcag tttctcgtca |
| 3781 | gctggtgtta gccgggcgga gtgtgttagt ctctccagag ggaggctggt tccccaggga |
| 3841 | agcagagcct gtgtgcgggc agcagctgtg tgcgggcctg ggggttgtta agtgcaatta |
| 3901 | tttttaataa aaggggcatt tggaaaaaaa aaaaaaaggt agcagtcgac agatgaattc |
| 3961 | tgcagatctg tggcttctag ctgcccgggt ggcatccctg tgacccctcc ccagtgcctc |
| 4021 | tcctggccct ggaagttgcc actccagtgc ccaccagcct tgtcctaata aaattaagtt |
| 4081 | gcatcatttt gtctgactag gtgtccttct ataatattat ggggtggagg ggggtggtat |
| 4141 | ggagcaaggg gcaagttggg aagacaacct gagttgttgg gattccaggc atcgagtaga |
| 4201 | taagtagcat ggcgggttaa tcattaacta caaggaaccc ctagtgatgg agttggccac |
| 4261 | tccctctctg cgcgctcgct cgctcactga ggccgggcga ccaaaggtcg cccgacgccc |
| 4321 | gggctttgcc cgggcggcct cagtgagcga gcgagcgcgc agagagggac agatccgaag |
| 4381 | ccgggcaaat cagcgcctgg cagcagtggc gtctggcgga aaacctcagt gtgacgctcc |
| 4441 | ccgccgcgtc ccacgcttgt tcccggatct gaccaccagc gaaatccgat ttttgcaccg |
| 4501 | agctgggtaa taagcgttgg caatttaacc gccagtcagg ctttctttca cagtgtggat |
| 4561 | tggcgataaa aaacaactgc tgacgccgct gcgcgatcag ttcacccgtt caccgctgga |
| 4621 | taacgacttg gcgtaagtga agcgacccgt aagaccctaa cgcctgggtc gaacgctgga |
| 4681 | aggcggcggg ccaaaccagg ccgaagcagc gttgttgcag ttcacggcag atacacttgc |
| 4741 | tgttgcggtg ctgattacga ccgctcactc gtggcagcaa caggggaaaa ccttatttat |
| 4801 | cagccggaaa acctaccgga ttgttggtag tggtcaatag gcgattaccg ttgtgttgaa |
| 4861 | gtggcgagcg atacaccgct tccggcgcgg attggcctga actgccaact ggcgcaggta |
| 4921 | gcagagcggg taaactggct cggattaggg ccgcaagaaa actatcccga ccgccttact |
| 4981 | gccgcctgtt ttgaccgctg ggatctgcca agtcagacag tatagcccgt acgtcttccc |
| 5041 | gagcgaaaac ggtctgcgct gcgggacgcg cgaattgaat ttggcccaca ccagtggcgc |
| 5101 | ggcgacttcc agttcaatat cagccgctac agtgaacagc aactgttgga aaccagcctt |
| 5161 | cgccaactgc tgcacgcgga agaaggcact ggctgaatat cgacggtttc cagttgggga |
| 5221 | ttggtggcga cgactcctgg agcccgtcag tatcggcgga cttccaactg agcgccggtc |
| 5281 | gctaccttac cagttggtct ggtgtcaaaa agcgtccgct tgagtctagc gatcgcgcgc |
| 5341 | agatctgtca tgtgagcaaa aggccagcaa aaggccagga accgtaaaaa ggccgcgttg |
| 5401 | ctggcgtttt tccataggct ccgcccccct gacgagcatc acaaaaatcg acgctcaagt |
| 5461 | cagaggtggc gaaacccgac aggactataa agataccagg cgtttccccc tggaagctcc |
| 5521 | ctcgtgcgct ctcctgttcc gaccctgccg cttaccggat acctgtccgc ctttctccct |
| 5581 | tcgggaagcg tggcgctttc tcatagctca cgctgtaggt atctcagttc ggtgtaggtc |
| 5641 | gttcgctcca agctgggctg tgtgcacgaa ccccccgttc agcccgaccg ctgcgcctta |
| 5701 | tccggtaact atcgtcttga gtccaacccg gtaagacacg acttatcgcc actggcagca |
| 5761 | gccactggta acaggattag cagagcgagg tatgtaggcg gtgctacaga gttcttgaag |
| 5821 | tggtggccta actacggcta cactagaaga acagtatttg gtatctgcgc tctgctgaag |
| 5881 | ccagttacct tcggaaaaag agttggtagc tcttgatccg gcaaacaaac caccgctggt |
| 5941 | agcggtggtt tttttgtttg caagcagcag attacgcgca gaaaaaaagg atctcaagaa |
| 6001 | gatcctttga tcttttctac ggggtctgac gctcagtgga acgaaaactc acgttaaggg |
| 6061 | attttggtca tgagattatc aaaaaggatc ttcacctaga tccttttcac gtagaaagcc |
| 6121 | agtccgcaga aacggtgctg accccggatg aatgtcagct actgggctat ctggacaagg |
| 6181 | gaaaacgcaa gcgcaaagag aaagcaggta gcttgcagtg ggcttacatg gcgatagcta |
| 6241 | gactgggcgg ttttatggac agcaagcgaa ccggaattgc cagctggggc gccctctggt |
| 6301 | aaggttggga agccctgcaa agtaaactgg atggctttct tgccgccaag gatctgatgg |
| 6361 | cgcaggggat caagatctga tcaagagaca ggatgaggat cgtttcgcat gattgaacaa |
| 6421 | gatggattgc acgcaggttc tccggccgct tgggtggaga ggctattcgg ctatgactgg |
| 6481 | gcacaacaga caatcggctg ctctgatgcc gccgtgttcc ggctgtcagc gcaggggcgc |
| 6541 | ccggttcttt ttgtcaagac cgacctgtcc ggtgccctga atgaactgca agacgaggca |
| 6601 | gcgcggctat cgtggctggc cacgacgggc gttccttgcg cagctgtgct cgacgttgtc |
| 6661 | actgaagcgg gaagggactg gctgctattg ggcgaagtgc cggggcagga tctcctgtca |
| 6721 | tctcaccttg ctcctgccga gaaagtatcc atcatggctg atgcaatgcg gcggctgcat |
| 6781 | acgcttgatc cggctacctg cccattcgac caccaagcga aacatcgcat cgagcgagca |
| 6841 | cgtactcgga tggaagccgg tcttgtcgat caggatgatc tggacgaaga gcatcagggg |
| 6901 | ctcgcgccag ccgaactgtt cgccaggctc aaggcgagca tgcccgacgg cgaggatctc |
| 6961 | gtcgtgaccc atggcgatgc ctgcttgccg aatatcatgg tggaaaatgg ccgcttttct |
| 7021 | ggattcatcg actgtggccg gctgggtgtg gcggaccgct atcaggacat agcgttggct |
| 7081 | acccgtgata ttgctgaaga gcttggcggc gaatgggctg accgcttcct cgtgctttac |
| 7141 | ggtatcgccg ctcccgattc gcagcgcatc gccttctatc gccttcttga cgagttcttc |
| 7201 | tgaatttaaa gcccaatacg caaaccgcct ctccccgcgc gttggccgaa gcggttcaat |
| 7261 | attttgttaa aattcgcgtt aaatttttgt taaatcagct attttttaac caataggccg |
| 7321 | aaatcggcaa aatcccttgt aaatcaaaag aatagaccga gatagggttg agtgttgttc |
| 7381 | cagtttggaa caagagtcca ctattaaaga acgtggactc caacgtcaaa gggcgaaaaa |
| 7441 | ccgtctatca gggcgttggc ccactacgtg aaccttcacc ctaatcaagt tttttggggt |
| 7501 | cgaggtgccg taaagcacta aatcggaacc ctaaagggag cccccgattt agagcttgac |
| 7561 | ggggaaaccg gcgaacgtgg cgagaaagga agggaagaaa gcgaaaggag cgggcgctag |
| 7621 | ggcgctggca agtgtagcgg tcacgctgcg cgtaaccacc acacccgccg cgctaagcgc |
| 7681 | cgctacaggg cgcgtccctt cgccttcagg ctgcgtcgag tactgtactg tgagccagag |
| 7741 | ttgcccggcg ctctccggct gcggtagttc aggcagttca atcaactgtt taccttgtgg |
| 7801 | agcgactcca gaggcacttc accgcttgcc agcggcttac gatccagcgc cacgatccag |
| 7861 | tgcaggagat cgttatcgct atacggaaca ggtattcgct ggtcacttcg ataaggtttg |
| 7921 | cccggataaa cggaactgga aaaactgctg ctggtgtttt gcttccgtca gtgctggatc |
| 7981 | ggcgtgcggt cggcaaagac cagaccgttc taacagaact ggcgattgtt cggcgtatcg |
| 8041 | ccaaaatcac cgccgtaagc cgaccacggg ttgccgtttt cagcaggatt taatcagcga |
| 8101 | ctgatccacc cagtcccaga cgaagccgcc ctgtaaacgg ggatactgac gaaacgcctg |
| 8161 | ccagtattta gcgaaaccgc caagactgtt acccaagcgt gggcgtattc gcaaaggatc |
| 8221 | agcgggcgcg tctctccagg tagcgaaagc cttttttgat cgacctttcg gcacagccgg |
| 8281 | gaagggctgg tcttcaacca cgcgcgcgta caacgggcaa ataatatcgg tggccgtggt |
| 8341 | gtcggctccg ccgccttcaa ctgcaccggg cgggaaggat cgacagattt gatccagcga |
| 8401 | tacagcgcgt cgtgattagc gccgtggcct gattcaattc cccagcgacc agtagatcac |
| 8461 | actcgggtga ttacgattgc gctgcaccag tcgcgttacg gttcgctctt cgccggtagc |
| 8521 | cagcgcggat cacggtcaga cgattcgttg gcacgatccg tgggtttcaa tactggcttc |
| 8581 | aaaccaccac taacaggccg tagcggtcgc acagcgtgta ccacagcggt tggttcggat |
| 8641 | aatcgaacag cgcacggcgt taaagttgtt ctgcttcaac agcaggatat tctgcacctt |
| 8701 | cgtctgctct tcctaacctg accaagcaga ggatctgctc gtgacggtta atcctcgaat |
| 8761 | cagcaacggc ttgccgttca gcagcagcag accaagttca atccgcacct cgcggaaacc |
| 8821 | gacaacgcag gcttctgctt caatcagcgt gccgtcggcg gtgtgcagtt caaccaccgc |
| 8881 | acgatagaga ttcgggattt cggcgctcca cagtttcggg ttttcgacgt tcagacgtag |
| 8941 | tgtgacgcga tctgcaaacc accacgctca acgataattt caccgccgaa aggcgcggtg |
| 9001 | ccgctggcga cctgcgtttc accctgccag aaagaaactg ttacccgtag gtagtcacgc |
| 9061 | aactcgccgc acactgaact tcagcctcca gtacagcgcg gctgaaatcg tcttaaagcg |
| 9121 | agtggcaact ggaaatcgct gatttgtgta gtcggtttag cagcaacgag acttcacgga |
| 9181 | aaatccgcta atccgccaca gatcctgatc ttccagataa ctgccgtcac tccaacgcag |
| 9241 | caccttcacc gcgaggcggt tttctccggc gcgtaaaaat cgctcaggtc aaattcagac |
| 9301 | ggcaaacgac tgtcctggcc gtaaccgacc cagcgcccgt tgcaccacag attgaaacgc |
| 9361 | cgagtttacg cctcaaaaat aattcgcgtc tggccttcct gtagccagct ttcacaacta |
| 9421 | taatagtgag cgagtaacaa cccgtcggat tctccgtggg aacaaacggc ggattgaccg |
| 9481 | tatagggata ggttacgttg gtgtagtagg gcgctccgta accgtgctac tgccagtttg |
| 9541 | aggggacgac gacagtatcg gcctcaggaa gatcgcactc cagccagctt tccggcaccg |
| 9601 | cttctggtac tggaaaccag gcaaagcgcc tatcgcctat caggctgcac aactgttggg |
| 9661 | aagggcgatc tgtgcgggcc tcttcgctat tacgccagct tgcgaaaggg ggtagtgctg |
| 9721 | caaggcgatt aagttgggta acgccagggt tttcccagtc acgacgtgga tctgggccac |
| 9781 | tccctct |

| pP155 (SEQ ID NO: 511) | |
|---|---|
| (SEQ ID NO: 511) | |
| 1 | ttgcccatta tacgcgcgta taatggacta ttgtgtgctg atatctgtac acttaagggc |
| 121 | tagatcttag cttacgtcac tagagggtcc acgtttagtt tttaagatcc attgatctcc |
| 181 | taaacgctgc aagattcgca acctggtata cttagcctag gcgctaggtc ctagtgcagc |
| 241 | gggacttttt ttctaaagtc gttgagagga ggagtcgtca gaccagatag ctttgatgtc |
| 301 | ctgatcggaa ggatcgttgg cccccctgca ggcagctgtt aattaactgc gcgctcgctc |
| 361 | gctcactgag gccgcccggg caaagcccgg gcgtcgggcg acctttggtc gcccggcctc |
| 421 | agtgagcgag cgagcgcgca gagagggagt ggccaactcc atcactaggg gttccttgta |
| 481 | gttaatgatt aacccgccat gctacttatc tacggcgcgc cacgcgtgga ggggtggagt |
| 541 | cgtgacgtga attacgtcat agggttaggg aggtcggccg ctctaggagt taatttttaa |
| 601 | aaagcagtca aaagtccaag tgcccttgcg agcatttact ctctctgttt gctctggtta |
| 661 | ataatctcag gagcacaaac attccttact agttctagga gttaattttt aaaaagcagt |
| 721 | caaaagtcca agtgcccttg cgagcattta ctctctctgt ttgctctggt taataatctc |
| 781 | aggagcacaa acattcctta ctagttctag agcggccgcc agtgtgctgg aattcggctt |
| 841 | ttttagggct ggaagctacc tttgacatca tttcctctgc gaatgcatgt ataatttcta |
| 901 | cagaacctat tagaaaggat cacccagcct ctgcttttgt acaactttcc cttaaaaaac |
| 961 | tgccaatccc actgctgttt ggcccaatag tgagaacttt ttcctgctgc ctcttggtgc |
| 1021 | ttttgcctat ggcccctatt ctgcctgctg aagacactct tgccagcatg gacttaaacc |
| 1081 | cctccagctc tgacaatcct ctttctcttt tgttttacat gaagggtctg gcagccaaag |
| 1141 | caatcactca aagttcaaac cttatcattt tttgctttgt tcctcttggc cttggttttg |
| 1201 | tacatcagct ttgaaaatac catcccaggg ttaatgctgg ggttaattta taactgagag |
| 1261 | tgctctagtt ctgcaataca ggacatgcta taaaaatgga aagatgttgc tttctgagag |
| 1321 | atcagcttac atgtggtacc gagctcggat ccactagtaa cggccgccag tgtgctggaa |
| 1381 | ttcggctttc caggccatct ttttttatgg gagtgaggca cccgccctgc tcccaccggc |
| 1441 | tcctggccgt ctgcgccctc gtgtccttgg caaccgccat ggagtttggg ctgagctggg |
| 1501 | tctttctggt ggccctgctg aagggagtcc agtgtgagca gcagggagcc agcagaccag |
| 1561 | ggccccggga tgcccaggca caccccggcc gtcccagagc agtgcccaca cagtgcgacg |
| 1621 | tcccccccaa cagccgcttc gattgcgccc ctgacaaggc catcacccag gaacagtgcg |
| 1681 | aggcccgcgg ctgctgctac atccctgcaa agcaggggct gcagggagcc cagatggggc |
| 1741 | agccctggtg cttcttccca cccagctacc ccagctacaa gctggagaac ctgagctcct |
| 1801 | ctgaaatggg ctacacggcc accctgaccc gtaccacccc caccttcttc cccaaggaca |
| 1861 | tcctgaccct gcggctggac gtgatgatgg agactgagaa ccgcctccac ttcacgatca |
| 1921 | aagatccagc taacaggcgc tacgaggtgc ccttggagac cccgcgtgtc cacagccggg |
| 1981 | caccgtcccc actctacagc gtggagttct ccgaggagcc cttcggggtg atcgtgcacc |
| 2041 | ggcagctgga cggccgcgtg ctgctgaaca cgacggtggc gcccctgttc tttgcggacc |
| 2101 | agttccttca gctgtccacc tcgctgccct cgcagtatat cacaggcctc gccgagcacc |
| 2161 | tcagtcccct gatgctcagc accagctgga ccaggatcac cctgtggaac cgggaccttg |
| 2221 | cgcccacgcc cggtgcgaac ctctacgggt ctcacccttt ctacctggcg ctggaggacg |
| 2281 | gcgggtcggc acacggggtg ttcctgctaa acagcaatgc catggatgtg gtcctgcagc |
| 2341 | cgagccctgc ccttagctgg aggtcgacag gtgggatcct ggatgtctac atcttcctgg |
| 2401 | gcccagagcc caagagcgtg gtgcagcagt acctggacgt tgtgggatac ccgttcatgc |
| 2461 | cgccatactg gggcctgggc ttccacctgt gccgctgggg ctactcctcc accgctatca |
| 2521 | cccgccaggt ggtggagaac atgaccaggg cccacttccc cctggacgtc caatggaacg |
| 2581 | acctggacta catggactcc cggagggact tcacgttcaa caaggatggc ttccgggact |
| 2641 | tcccggccat ggtgcaggag ctgcaccagg gcggccggcg ctacatgatg atcgtggatc |
| 2701 | ctgccatcag cagctcgggc cctgccggga gctacaggcc ctacgacgag ggtctgcgga |
| 2761 | ggggggtttt catcaccaac gagaccggcc agccgctgat tgggaaggta tggcccgggt |
| 2821 | ccactgcctt ccccgacttc accaacccca cagccctggc ctggtgggag gacatggtgg |
| 2881 | ctgagttcca tgaccaggtg cccttcgacg gcatgtggat tgacatgaac gagccttcca |
| 2941 | acttcatcag aggctctgag gacggctgcc ccaacaatga gctggagaac ccaccctacg |
| 3001 | tgcctggggt ggttgggggg accctccagg cggccaccat ctgtgcctcc agccaccagt |
| 3061 | ttctctccac acactacaac ctgcacaacc tctacggcct gaccgaagcc atcgcctccc |
| 3121 | acagggcgct ggtgaaggct cgggggacac gcccatttgt gatctcccgc tcgacctttg |
| 3181 | ctggccacgg ccgatacgcc ggccactgga cgggggacgt gtggagctcc tgggagcagc |
| 3241 | tcgcctcctc cgtgccagaa atcctgcagt ttaacctgct gggggtgcct ctggtcgggg |
| 3301 | ccgacgtctg cggcttcctg ggcaacacct cagaggagct gtgtgtgcgc tggacccagc |
| 3361 | tgggggcctt ctaccccttc atgcggaacc acaacagcct gctcagtctg ccccaggagc |
| 3421 | cgtacagctt cagcgagccg gcccagcagg ccatgaggaa ggccctcacc ctgcgctacg |
| 3481 | cactcctccc ccacctctac acactgttcc accaggccca cgtcgcgggg gagaccgtgg |
| 3541 | cccggcccct cttcctggag ttccccaagg actctagcac ctggactgtg gaccaccagc |
| 3601 | tcctgtgggg ggaggccctg ctcatcaccc cagtgctcca ggccgggaag gccgaagtga |
| 3661 | ctggctactt ccccttgggc acatggtacg acctgcagac ggtgccaata gaggcccttg |
| 3721 | gcagcctccc acccccacct gcagctcccc gtgagccagc catccacagc gaggggcagt |
| 3781 | gggtgacgct gccggccccc ctggacacca tcaacgtcca cctccgggct gggtacatca |
| 3841 | tccccctgca gggccctggc ctcacaacca cagagtcccg ccagcagccc atggccctgg |
| 3901 | ctgtggccct gaccaagggt ggagaggccc gaggggagct gttctgggac gatggagaga |
| 3961 | gcctggaagt gctggagcga ggggcctaca cacaggtcat cttcctggcc aggaataaca |
| 4021 | cgatcgtgaa tgagctggta cgtgtgacca gtgagggagc tggcctgcag ctgcagaagg |
| 4081 | tgactgtcct gggcgtggcc acggcgcccc agcaggtcct ctccaacggt gtccctgtct |
| 4141 | ccaacttcac ctacagcccc gacaccaagg tcctggacat ctgtgtctcg ctgttgatgg |
| 4201 | gagagcagtt tctcgtcagc tggtgttagc cgggcggagt gtgttagtct ctccagaggg |
| 4261 | aggctggttc cccagggaag cagagcctgt gtgcgggcag cagctgtgtg cgggcctggg |
| 4321 | ggttgttaag tgcaattatt tttaataaaa ggggcatttg gaaaaaaaaa aaaaaggtag |
| 4381 | cagtcgacag atgaattctg cagatctgtg gcttctagct gcccgggtgg catccctgtg |
| 4441 | acccctcccc agtgcctctc ctggccctgg aagttgccac tccagtgccc accagccttg |
| 4501 | tcctaataaa attaagttgc atcattttgt ctgactaggt gtccttctat aatattatgg |
| 4561 | ggtggagggg ggtggtatgg agcaaggggc aagttgggaa gacaacctga gttgttggga |
| 4621 | ttccaggcat cgagtttaaa cgcggccgcg tagataagta gcatggcggg ttaatcatta |
| 4681 | actacaagga acccctagtg atggagttgg ccactccctc tctgcgcgct cgctcgctca |
| 4741 | ctgaggccgg gcgaccaaag gtcgcccgac gcccgggctt tgcccgggcg gcctcagtga |
| 4801 | gcgagcgagc gcgcagttaa ttaaggcgcc ctaggccgac ccttagactc tgtactcagt |
| 4861 | tctataaacg agccattgga tacgagatcc gtagattgat aagggacacg gaatatcccc |
| 4921 | ggacgcaata gacaccggtg gacagcttgg tatcctgagc acagtcgcgc gtccgaatct |
| 4981 | agctctactt tagaggcccc ggattctgat ggtcgtagac cgcagaaccg attgggggga |
| 5041 | tgagatctac tagttatcag cacacaattg cccattatac gcgcgtataa tggactattg |
| 5101 | tgtgctgata tagggataac agggtaattc tagagctagc atatggatcc atcgatttga |
| 5161 | tgcggtattt tctccttacg catctgtgcg gtatttcaca ccgcatacgt caaagcaacc |
| 5221 | atagtacgcg ccctgtagcg gcgcattaag cgcggcgggt gtggtggtta cgcgcagcgt |
| 5281 | gaccgctaca cttgccagcg ccctagcgcc cgctcctttc gctttcttcc cttcctttct |
| 5341 | cgccacgttc gccggctttc cccgtcaagc tctaaatcgg gggctccctt tagggttccg |
| 5401 | atttagtgct ttacggcacc tcgaccccaa aaaacttgat ttgggtgatg gttcacgtag |
| 5461 | tgggccatcg ccctgataga cggtttttcg ccctttgacg ttggagtcca cgttctttaa |
| 5521 | tagtggactc ttgttccaaa ctggaacaac actcaactct atctcgggct attcttttga |
| 5581 | tttataaggg attttgccga tttcggtcta ttggttaaaa aatgagctga tttaacaaaa |
| 5641 | atttaacgcg aattttaaca aaatattaac gtttacaatt ttatggtgca ctctcagtac |
| 5701 | aatctgctct gatgccgcat agttaagcca gccccgacac ccgccaacac ccgctgacgc |
| 5761 | gccctgacgg gcttgtctgc tcccggcatc cgcttacaga caagctgtga ccgtctccgg |
| 5821 | gagctgcatg tgtcagaggt tttcaccgtc atcaccgaaa cgcgcgagac gaaagggcct |
| 5881 | cgtgatacgc ctatttttat aggttaatgt catgataata atggtttctt agacgtcagg |
| 5941 | tggcactttt cggggaaatg tgcgcggaac ccctatttgt ttatttttct aaatacattc |
| 6001 | aaatatgtat ccgctcatga gacaataacc ctgataaatg cttcaataat attgaaaaag |
| 6061 | gaagagtatg agccatattc aacgggaaac gtcgaggccg cgattaaatt ccaacatgga |
| 6121 | tgctgattta tatgggtata aatgggctcg cgataatgtc gggcaatcag gtgcgacaat |
| 6181 | ctatcgcttg tatgggaagc ccgatgcgcc agagttgttt ctgaaacatg gcaaaggtag |
| 6241 | cgttgccaat gatgttacag atgagatggt cagactaaac tggctgacgg aatttatgcc |
| 6301 | tcttccgacc atcaagcatt ttatccgtac tcctgatgat gcatggttac tcaccactgc |
| 6361 | gatccccgga aaaacagcat tccaggtatt agaagaatat cctgattcag gtgaaaatat |
| 6421 | tgttgatgcg ctggcagtgt tcctgcgccg gttgcattcg attcctgttt gtaattgtcc |
| 6481 | ttttaacagc gatcgcgtat ttcgtctcgc tcaggcgcaa tcacgaatga ataacggttt |
| 6541 | ggttgatgcg agtgattttg atgacgagcg taatggctgg cctgttgaac aagtctggaa |
| 6601 | agaaatgcat aaacttttgc cattctcacc ggattcagtc gtcactcatg gtgatttctc |
| 6661 | acttgataac cttatttttg acgaggggaa attaataggt tgtattgatg ttggacgagt |
| 6721 | cggaatcgca gaccgatacc aggatcttgc catcctatgg aactgcctcg gtgagttttc |
| 6781 | tccttcatta cagaaacggc tttttcaaaa atatggtatt gataatcctg atatgaataa |
| 6841 | attgcagttt catttgatgc tcgatgagtt tttctaagcg tataatggtc tagagctagc |
| 6901 | atatggatcc atcgattcca ttatacgcct gtcagaccaa gtttactcat atatacttta |
| 6961 | gattgattta aaacttcatt tttaatttaa aaggatctag gtgaagatcc tttttgataa |
| 7021 | tctcatgacc aaaatccctt aacgtgagtt ttcgttccac tgagcgtcag accccgtaga |
| 7081 | aaagatcaaa ggatcttctt gagatccttt ttttctgcgc gtaatctgct gcttgcaaac |
| 7141 | aaaaaaacca ccgctaccag cggtggtttg tttgccggat caagagctac caactctttt |
| 7201 | tccgaaggta actggcttca gcagagcgca gataccaaat actgttcttc tagtgtagcc |
| 7261 | gtagttaggc caccacttca agaactctgt agcaccgcct acatacctcg ctctgctaat |
| 7321 | cctgttacca gtggctgctg ccagtggcga taagtcgtgt cttaccgggt tggactcaag |
| 7381 | acgatagtta ccggataagg cgcagcggtc gggctgaacg gggggttcgt gcacacagcc |
| 7441 | cagcttggag cgaacgacct acaccgaact gagataccta cagcgtgagc tatgagaaag |
| 7501 | cgccacgctt cccgaaggga gaaaggcgga caggtatccg gtaagcggca gggtcggaac |
| 7561 | aggagagcgc acgagggagc ttccaggggg aaacgcctgg tatctttata gtcctgtcgg |
| 7621 | gtttcgccac ctctgacttg agcgtcgatt tttgtgatgc tcgtcagggg ggcggagcct |
| 7681 | atggaaaaac gccagcaacg cggccttttt acggttcctg gccttttgct ggccttttgc |
| 7741 | tcacatgt |

| pP157 (SEQ ID NO: 512) | |
|---|---|
| (SEQ ID NO: 512) | |
| 1 | tctagagcta gcatatggat ccatcgattt agggataaca gggtaattat cagcacacaa |
| 61 | ttgcccatta tacgcgcgta taatggacta ttgtgtgctg atatctgtac acttaagggc |
| 121 | tagatcttag cttacgtcac tagagggtcc acgtttagtt tttaagatcc attgatctcc |
| 181 | taaacgctgc aagattcgca acctggtata cttagcctag gcgctaggtc ctagtgcagc |
| 241 | gggacttttt ttctaaagtc gttgagagga ggagtcgtca gaccagatag ctttgatgtc |
| 301 | ctgatcggaa ggatcgttgg cccccctgca ggcagctgtt aattaactgc gcgctcgctc |
| 361 | gctcactgag gccgcccggg caaagcccgg gcgtcgggcg acctttggtc gcccggcctc |
| 421 | agtgagcgag cgagcgcgca gagagggagt ggccaactcc atcactaggg gttccttgta |
| 481 | gttaatgatt aacccgccat gctacttatc tacggcgcgc cacgcgtgga ggggtggagt |
| 541 | cgtgacgtga attacgtcat agggttaggg aggtcggccg ctctaggagt taatttttaa |
| 601 | aaagcagtca aaagtccaag tgcccttgcg agcatttact ctctctgttt gctctggtta |
| 661 | ataatctcag gagcacaaac attccttact agttctagga gttaattttt aaaaagcagt |
| 721 | caaaagtcca agtgcccttg cgagcattta ctctctctgt ttgctctggt taataatctc |
| 781 | aggagcacaa acattcctta ctagttctag agcggccgcc agtgtgctgg aattcggctt |
| 841 | ttttagggct ggaagctacc tttgacatca tttcctctgc gaatgcatgt ataatttcta |
| 901 | cagaacctat tagaaaggat cacccagcct ctgcttttgt acaactttcc cttaaaaaac |
| 961 | tgccaatccc actgctgttt ggcccaatag tgagaacttt ttcctgctgc ctcttggtgc |
| 1021 | ttttgcctat ggcccctatt ctgcctgctg aagacactct tgccagcatg gacttaaacc |
| 1081 | cctccagctc tgacaatcct ctttctcttt tgttttacat gaagggtctg gcagccaaag |
| 1141 | caatcactca aagttcaaac cttatcattt tttgctttgt tcctcttggc cttggttttg |
| 1201 | tacatcagct ttgaaaatac catcccaggg ttaatgctgg ggttaattta taactgagag |
| 1261 | tgctctagtt ctgcaataca ggacatgcta taaaaatgga aagatgttgc tttctgagag |
| 1321 | atcagcttac atgtggtacc gagctcggat ccactagtaa cggccgccag tgtgctggaa |
| 1381 | ttcggctttc caggccatct ccaaccatgg gagtgaggca cccgccctgc tcccaccggc |
| 1441 | tcctggccgt ctgcgccctc gtgtccttgg caaccgctgc actcctgggg cagcagggag |
| 1501 | ccagcagacc agggccccgg gatgcccagg cacaccccgg ccgtcccaga gcagtgccca |
| 1561 | cacagtgcga cgtccccccc aacagccgct tcgattgcgc ccctgacaag gccatcaccc |
| 1621 | aggaacagtg cgaggcccgc ggctgctgct acatccctgc aaagcagggg ctgcagggag |
| 1681 | cccagatggg gcagccctgg tgcttcttcc cacccagcta ccccagctac aagctggaga |
| 1741 | acctgagctc ctctgaaatg ggctacacgg ccaccctgac ccgtaccacc cccaccttct |
| 1801 | tccccaagga catcctgacc ctgcggctgg acgtgatgat ggagactgag aaccgcctcc |
| 1861 | acttcacgat caaagatcca gctaacaggc gctacgaggt gcccttggag accccgcgtg |
| 1921 | tccacagccg ggcaccgtcc ccactctaca gcgtggagtt ctccgaggag cccttcgggg |
| 1981 | tgatcgtgca ccggcagctg gacggccgcg tgctgctgaa cacgacggtg gcgcccctgt |
| 2041 | tctttgcgga ccagttcctt cagctgtcca cctcgctgcc ctcgcagtat atcacaggcc |
| 2101 | tcgccgagca cctcagtccc ctgatgctca gcaccagctg gaccaggatc accctgtgga |
| 2161 | accgggacct tgcgcccacg cccggtgcga acctctacgg gtctcaccct ttctacctgg |
| 2221 | cgctggagga cggcgggtcg gcacacgggg tgttcctgct aaacagcaat gccatggatg |
| 2281 | tggtcctgca gccgagccct gcccttagct ggaggtcgac aggtgggatc ctggatgtct |
| 2341 | acatcttcct gggcccagag cccaagagcg tggtgcagca gtacctggac gttgtgggat |
| 2401 | acccgttcat gccgccatac tggggcctgg gcttccacct gtgccgctgg ggctactcct |
| 2461 | ccaccgctat cacccgccag gtggtggaga acatgaccag ggcccacttc cccctggacg |
| 2521 | tccaatggaa cgacctggac tacatggact cccggaggga cttcacgttc aacaaggatg |
| 2581 | gcttccggga cttcccggcc atggtgcagg agctgcacca gggcggccgg cgctacatga |
| 2641 | tgatcgtgga tcctgccatc agcagctcgg gccctgccgg gagctacagg ccctacgacg |
| 2701 | agggtctgcg gaggggggtt ttcatcacca acgagaccgg ccagccgctg attgggaagg |
| 2761 | tatggcccgg gtccactgcc ttccccgact tcaccaaccc cacagccctg gcctggtggg |
| 2821 | aggacatggt ggctgagttc catgaccagg tgcccttcga cggcatgtgg attgacatga |
| 2881 | acgagccttc caacttcatc agaggctctg aggacggctg ccccaacaat gagctggaga |
| 2941 | acccacccta cgtgcctggg gtggttgggg ggaccctcca ggcggccacc atctgtgcct |
| 3001 | ccagccacca gtttctctcc acacactaca acctgcacaa cctctacggc ctgaccgaag |
| 3061 | ccatcgcctc ccacagggcg ctggtgaagg ctcgggggac acgcccattt gtgatctccc |
| 3121 | gctcgacctt tgctggccac ggccgatacg ccggccactg gacgggggac gtgtggagct |
| 3181 | cctgggagca gctcgcctcc tccgtgccag aaatcctgca gtttaacctg ctgggggtgc |
| 3241 | ctctggtcgg ggccgacgtc tgcggcttcc tgggcaacac ctcagaggag ctgtgtgtgc |
| 3301 | gctggaccca gctgggggcc ttctacccct tcatgcggaa ccacaacagc ctgctcagtc |
| 3361 | tgccccagga gccgtacagc ttcagcgagc cggcccagca ggccatgagg aaggccctca |
| 3421 | ccctgcgcta cgcactcctc ccccacctct acacactgtt ccaccaggcc cacgtcgcgg |
| 3481 | gggagaccgt ggcccggccc ctcttcctgg agttccccaa ggactctagc acctggactg |
| 3541 | tggaccacca gctcctgtgg ggggaggccc tgctcatcac cccagtgctc caggccggga |
| 3601 | aggccgaagt gactggctac ttccccttgg gcacatggta cgacctgcag acggtgccaa |
| 3661 | tagaggccct tggcagcctc ccacccccac ctgcagctcc ccgtgagcca gccatccaca |
| 3721 | gcgaggggca gtgggtgacg ctgccggccc ccctggacac catcaacgtc cacctccggg |
| 3781 | ctgggtacat catccccctg cagggccctg gcctcacaac cacagagtcc cgccagcagc |
| 3841 | ccatggccct ggctgtggcc ctgaccaagg gtggagaggc ccgaggggag ctgttctggg |
| 3901 | acgatggaga gagcctggaa gtgctggagc gaggggccta cacacaggtc atcttcctgg |
| 3961 | ccaggaataa cacgatcgtg aatgagctgg tacgtgtgac cagtgaggga gctggcctgc |
| 4021 | agctgcagaa ggtgactgtc ctgggcgtgg ccacggcgcc ccagcaggtc ctctccaacg |
| 4081 | gtgtccctgt ctccaacttc acctacagcc ccgacaccaa ggtcctggac atctgtgtct |
| 4141 | cgctgttgat gggagagcag tttctcgtca gctggtgtta gccgggcgga gtgtgttagt |
| 4201 | ctctccagag ggaggctggt tccccaggga agcagagcct gtgtgcgggc agcagctgtg |
| 4261 | tgcgggcctg ggggttgtta agtgcaatta tttttaataa aaggggcatt tggaaaaaaa |
| 4321 | aaaaaaaggt agcagtcgac agatgaattc tgcagatctg tggcttctag ctgcccgggt |
| 4381 | ggcatccctg tgacccctcc ccagtgcctc tcctggccct ggaagttgcc actccagtgc |
| 4441 | ccaccagcct tgtcctaata aaattaagtt gcatcatttt gtctgactag gtgtccttct |
| 4501 | ataatattat ggggtggagg ggggtggtat ggagcaaggg gcaagttggg aagacaacct |
| 4561 | gagttgttgg gattccaggc atcgagttta aacgcggccg cgtagataag tagcatggcg |
| 4621 | ggttaatcat taactacaag gaacccctag tgatggagtt ggccactccc tctctgcgcg |
| 4681 | ctcgctcgct cactgaggcc gggcgaccaa aggtcgcccg acgcccgggc tttgcccggg |
| 4741 | cggcctcagt gagcgagcga gcgcgcagtt aattaaggcg ccctaggccg acccttagac |
| 4801 | tctgtactca gttctataaa cgagccattg gatacgagat ccgtagattg ataagggaca |
| 4861 | cggaatatcc ccggacgcaa tagacaccgg tggacagctt ggtatcctga gcacagtcgc |
| 4921 | gcgtccgaat ctagctctac tttagaggcc ccggattctg atggtcgtag accgcagaac |
| 4981 | cgattggggg gatgagatct actagttatc agcacacaat tgcccattat acgcgcgtat |
| 5041 | aatggactat tgtgtgctga tatagggata acagggtaat tctagagcta gcatatggat |
| 5101 | ccatcgattt gatgcggtat tttctcctta cgcatctgtg cggtatttca caccgcatac |
| 5161 | gtcaaagcaa ccatagtacg cgccctgtag cggcgcatta agcgcggcgg gtgtggtggt |
| 5221 | tacgcgcagc gtgaccgcta cacttgccag cgccctagcg cccgctcctt tcgctttctt |
| 5281 | cccttccttt ctcgccacgt tcgccggctt tccccgtcaa gctctaaatc gggggctccc |
| 5341 | tttagggttc cgatttagtg ctttacggca cctcgacccc aaaaaacttg atttgggtga |
| 5401 | tggttcacgt agtgggccat cgccctgata gacggttttt cgccctttga cgttggagtc |
| 5461 | cacgttcttt aatagtggac tcttgttcca aactggaaca acactcaact ctatctcggg |
| 5521 | ctattctttt gatttataag ggattttgcc gatttcggtc tattggttaa aaaatgagct |
| 5581 | gatttaacaa aaatttaacg cgaattttaa caaaatatta acgtttacaa ttttatggtg |
| 5641 | cactctcagt acaatctgct ctgatgccgc atagttaagc cagccccgac acccgccaac |
| 5701 | acccgctgac gcgccctgac gggcttgtct gctcccggca tccgcttaca gacaagctgt |
| 5761 | gaccgtctcc gggagctgca tgtgtcagag gttttcaccg tcatcaccga aacgcgcgag |
| 5821 | acgaaagggc ctcgtgatac gcctattttt ataggttaat gtcatgataa taatggtttc |
| 5881 | ttagacgtca ggtggcactt ttcggggaaa tgtgcgcgga acccctattt gtttattttt |
| 5941 | ctaaatacat tcaaatatgt atccgctcat gagacaataa ccctgataaa tgcttcaata |
| 6001 | atattgaaaa aggaagagta tgagccatat tcaacgggaa acgtcgaggc cgcgattaaa |
| 6061 | ttccaacatg gatgctgatt tatatgggta taaatgggct cgcgataatg tcgggcaatc |
| 6121 | aggtgcgaca atctatcgct tgtatgggaa gcccgatgcg ccagagttgt ttctgaaaca |
| 6181 | tggcaaaggt agcgttgcca atgatgttac agatgagatg gtcagactaa actggctgac |
| 6241 | ggaatttatg cctcttccga ccatcaagca ttttatccgt actcctgatg atgcatggtt |
| 6301 | actcaccact gcgatccccg gaaaaacagc attccaggta ttagaagaat atcctgattc |
| 6361 | aggtgaaaat attgttgatg cgctggcagt gttcctgcgc cggttgcatt cgattcctgt |
| 6421 | ttgtaattgt ccttttaaca gcgatcgcgt atttcgtctc gctcaggcgc aatcacgaat |
| 6481 | gaataacggt ttggttgatg cgagtgattt tgatgacgag cgtaatggct ggcctgttga |
| 6541 | acaagtctgg aaagaaatgc ataaactttt gccattctca ccggattcag tcgtcactca |
| 6601 | tggtgatttc tcacttgata accttatttt tgacgagggg aaattaatag gttgtattga |
| 6661 | tgttggacga gtcggaatcg cagaccgata ccaggatctt gccatcctat ggaactgcct |
| 6721 | cggtgagttt tctccttcat tacagaaacg gctttttcaa aaatatggta ttgataatcc |
| 6781 | tgatatgaat aaattgcagt ttcatttgat gctcgatgag tttttctaag cgtataatgg |
| 6841 | tctagagcta gcatatggat ccatcgattc cattatacgc ctgtcagacc aagtttactc |
| 6901 | atatatactt tagattgatt taaaacttca tttttaattt aaaaggatct aggtgaagat |
| 6961 | cctttttgat aatctcatga ccaaaatccc ttaacgtgag ttttcgttcc actgagcgtc |
| 7021 | agaccccgta gaaaagatca aaggatcttc ttgagatcct ttttttctgc gcgtaatctg |
| 7081 | ctgcttgcaa acaaaaaaac caccgctacc agcggtggtt tgtttgccgg atcaagagct |
| 7141 | accaactctt tttccgaagg taactggctt cagcagagcg cagataccaa atactgttct |
| 7201 | tctagtgtag ccgtagttag gccaccactt caagaactct gtagcaccgc ctacatacct |
| 7261 | cgctctgcta atcctgttac cagtggctgc tgccagtggc gataagtcgt gtcttaccgg |
| 7321 | gttggactca agacgatagt taccggataa ggcgcagcgg tcgggctgaa cggggggttc |
| 7381 | gtgcacacag cccagcttgg agcgaacgac ctacaccgaa ctgagatacc tacagcgtga |
| 7441 | gctatgagaa agcgccacgc ttcccgaagg gagaaaggcg gacaggtatc cggtaagcgg |
| 7501 | cagggtcgga acaggagagc gcacgaggga gcttccaggg ggaaacgcct ggtatcttta |
| 7561 | tagtcctgtc gggtttcgcc acctctgact tgagcgtcga tttttgtgat gctcgtcagg |
| 7621 | ggggcggagc ctatggaaaa acgccagcaa cgcggccttt ttacggttcc tggccttttg |
| 7681 | ctggcctttt gctcacatgt |

| ITR to ITR sequence of pP110, i.e., amino acids 1-4385 of SEQ ID NO: 502. | |
|---|---|
| (SEQ ID NO: 513) | |
| (SEQ ID NO: 513) | |
| 1 | tgggccactc cctctctgcg cgctcgctcg ctcactgagg ccgcccgggc aaagcccggg |
| 61 | cgtcgggcga cctttggtcg cccggcctca gtgagcgagc gagcgcgcag agagggagtg |
| 121 | gccaactcca tcactagggg ttcctggagg ggtggagtcg tgacgtgaat tacgtcatag |
| 181 | ggttagggag gtcggccgct ctaggagtta atttttaaaa agcagtcaaa agtccaagtg |
| 241 | cccttgcgag catttactct ctctgtttgc tctggttaat aatctcagga gcacaaacat |
| 301 | tccttactag ttctaggagt taatttttaa aaagcagtca aaagtccaag tgcccttgcg |
| 361 | agcatttact ctctctgttt gctctggtta ataatctcag gagcacaaac attccttact |
| 421 | agttctagag cggccgccag tgtgctggaa ttcggctttt ttagggctgg aagctacctt |
| 481 | tgacatcatt tcctctgcga atgcatgtat aatttctaca gaacctatta gaaaggatca |
| 541 | cccagcctct gcttttgtac aactttccct taaaaaactg ccaatcccac tgctgtttgg |
| 601 | cccaatagtg agaacttttt cctgctgcct cttggtgctt ttgcctatgg cccctattct |
| 661 | gcctgctgaa gacactcttg ccagcatgga cttaaacccc tccagctctg acaatcctct |
| 721 | ttctcttttg ttttacatga agggtctggc agccaaagca atcactcaaa gttcaaacct |
| 781 | tatcattttt tgctttgttc ctcttggcct tggttttgta catcagcttt gaaaatacca |
| 841 | tcccagggtt aatgctgggg ttaatttata actgagagtg ctctagttct gcaatacagg |
| 901 | acatgctata aaaatggaaa gatgttgctt tctgagagat cagcttacat gtggtaccga |
| 961 | gctcggatcc actagtaacg gccgccagtg tgctggaatt cggctttcca ggccatctcc |
| 1021 | aaccatggga gtgaggcacc cgccctgctc ccaccggctc ctggccgtct gcgccctcgt |
| 1081 | gtccttggca accgctgcac tcctggggca gcagggagcc agcagaccag ggccccggga |
| 1141 | tgcccaggca caccccggcc gtcccagagc agtgcccaca cagtgcgacg tcccccccaa |
| 1201 | cagccgcttc gattgcgccc ctgacaaggc catcacccag gaacagtgcg aggcccgcgg |
| 1261 | ctgctgctac atccctgcaa agcaggggct gcagggagcc cagatggggc agccctggtg |
| 1321 | cttcttccca cccagctacc ccagctacaa gctggagaac ctgagctcct ctgaaatggg |
| 1381 | ctacacggcc accctgaccc gtaccacccc caccttcttc cccaaggaca tcctgaccct |
| 1441 | gcggctggac gtgatgatgg agactgagaa ccgcctccac ttcacgatca aagatccagc |
| 1501 | taacaggcgc tacgaggtgc ccttggagac cccgcgtgtc cacagccggg caccgtcccc |
| 1561 | actctacagc gtggagttct ccgaggagcc cttcggggtg atcgtgcacc ggcagctgga |
| 1621 | cggccgcgtg ctgctgaaca cgacggtggc gcccctgttc tttgcggacc agttccttca |
| 1681 | gctgtccacc tcgctgccct cgcagtatat cacaggcctc gccgagcacc tcagtcccct |
| 1741 | gatgctcagc accagctgga ccaggatcac cctgtggaac cgggaccttg cgcccacgcc |
| 1801 | cggtgcgaac ctctacgggt ctcacccttt ctacctggcg ctggaggacg gcgggtcggc |
| 1861 | acacggggtg ttcctgctaa acagcaatgc catggatgtg gtcctgcagc cgagccctgc |
| 1921 | ccttagctgg aggtcgacag gtgggatcct ggatgtctac atcttcctgg gcccagagcc |
| 1981 | caagagcgtg gtgcagcagt acctggacgt tgtgggatac ccgttcatgc cgccatactg |
| 2041 | gggcctgggc ttccacctgt gccgctgggg ctactcctcc accgctatca cccgccaggt |
| 2101 | ggtggagaac atgaccaggg cccacttccc cctggacgtc caatggaacg acctggacta |
| 2161 | catggactcc cggagggact tcacgttcaa caaggatggc ttccgggact tcccggccat |
| 2221 | ggtgcaggag ctgcaccagg gcggccggcg ctacatgatg atcgtggatc ctgccatcag |
| 2281 | cagctcgggc cctgccggga gctacaggcc ctacgacgag ggtctgcgga ggggggtttt |
| 2341 | catcaccaac gagaccggcc agccgctgat tgggaaggta tggcccgggt ccactgcctt |
| 2401 | ccccgacttc accaacccca cagccctggc ctggtgggag gacatggtgg ctgagttcca |
| 2461 | tgaccaggtg cccttcgacg gcatgtggat tgacatgaac gagccttcca acttcatcag |
| 2521 | aggctctgag gacggctgcc ccaacaatga gctggagaac ccaccctacg tgcctggggt |
| 2581 | ggttgggggg accctccagg cggccaccat ctgtgcctcc agccaccagt ttctctccac |
| 2641 | acactacaac ctgcacaacc tctacggcct gaccgaagcc atcgcctccc acagggcgct |
| 2701 | ggtgaaggct cgggggacac gcccatttgt gatctcccgc tcgacctttg ctggccacgg |
| 2761 | ccgatacgcc ggccactgga cgggggacgt gtggagctcc tgggagcagc tcgcctcctc |
| 2821 | cgtgccagaa atcctgcagt ttaacctgct gggggtgcct ctggtcgggg ccgacgtctg |
| 2881 | cggcttcctg ggcaacacct cagaggagct gtgtgtgcgc tggacccagc tgggggcctt |
| 2941 | ctaccccttc atgcggaacc acaacagcct gctcagtctg ccccaggagc cgtacagctt |
| 3001 | cagcgagccg gcccagcagg ccatgaggaa ggccctcacc ctgcgctacg cactcctccc |
| 3061 | ccacctctac acactgttcc accaggccca cgtcgcgggg gagaccgtgg cccggcccct |
| 3121 | cttcctggag ttccccaagg actctagcac ctggactgtg gaccaccagc tcctgtgggg |
| 3181 | ggaggccctg ctcatcaccc cagtgctcca ggccgggaag gccgaagtga ctggctactt |
| 3241 | ccccttgggc acatggtacg acctgcagac ggtgccaata gaggcccttg gcagcctccc |
| 3301 | acccccacct gcagctcccc gtgagccagc catccacagc gaggggcagt gggtgacgct |
| 3361 | gccggccccc ctggacacca tcaacgtcca cctccgggct gggtacatca tccccctgca |
| 3421 | gggccctggc ctcacaacca cagagtcccg ccagcagccc atggccctgg ctgtggccct |
| 3481 | gaccaagggt ggagaggccc gaggggagct gttctgggac gatggagaga gcctggaagt |
| 3541 | gctggagcga ggggcctaca cacaggtcat cttcctggcc aggaataaca cgatcgtgaa |
| 3601 | tgagctggta cgtgtgacca gtgagggagc tggcctgcag ctgcagaagg tgactgtcct |
| 3661 | gggcgtggcc acggcgcccc agcaggtcct ctccaacggt gtccctgtct ccaacttcac |
| 3721 | ctacagcccc gacaccaagg tcctggacat ctgtgtctcg ctgttgatgg gagagcagtt |
| 3781 | tctcgtcagc tggtgttagc cgggcggagt gtgttagtct ctccagaggg aggctggttc |
| 3841 | cccagggaag cagagcctgt gtgcgggcag cagctgtgtg cgggcctggg ggttgttaag |
| 3901 | tgcaattatt tttaataaaa ggggcatttg gaaaaaaaaa aaaaaggtag cagtcgacag |
| 3961 | atgaattctg cagatctgtg gcttctagct gcccgggtgg catccctgtg acccctcccc |
| 4021 | agtgcctctc ctggccctgg aagttgccac tccagtgccc accagccttg tcctaataaa |
| 4081 | attaagttgc atcattttgt ctgactaggt gtccttctat aatattatgg ggtggagggg |
| 4141 | ggtggtatgg agcaaggggc aagttgggaa gacaacctga gttgttggga ttccaggcat |
| 4201 | cgagtagata agtagcatgg cgggttaatc attaactaca aggaacccct agtgatggag |
| 4261 | ttggccactc cctctctgcg cgctcgctcg ctcactgagg ccgggcgacc aaaggtcgcc |
| 4321 | cgacgcccgg gctttgcccg ggcggcctca gtgagcgagc gagcgcgcag agagggacag |
| 4381 | atccg |

| ITR to ITR sequence of pP112, i.e., amino acids 1- 4439 of SEQ ID NO: 504. | |
|---|---|
| (SEQ ID NO: 514) | |
| (SEQ ID NO: 514) | |
| 1 | tgggccactc cctctctgcg cgctcgctcg ctcactgagg ccgcccgggc aaagcccggg |
| 61 | cgtcgggcga cctttggtcg cccggcctca gtgagcgagc gagcgcgcag agagggagtg |
| 121 | gccaactcca tcactagggg ttcctggagg ggtggagtcg tgacgtgaat tacgtcatag |
| 181 | ggttagggag gtcggccgct ctaggagtta atttttaaaa agcagtcaaa agtccaagtg |
| 241 | cccttgcgag catttactct ctctgtttgc tctggttaat aatctcagga gcacaaacat |
| 301 | tccttactag ttctaggagt taatttttaa aaagcagtca aaagtccaag tgcccttgcg |
| 361 | agcatttact ctctctgttt gctctggtta ataatctcag gagcacaaac attccttact |
| 421 | agttctagag cggccgccag tgtgctggaa ttcggctttt ttagggctgg aagctacctt |
| 481 | tgacatcatt tcctctgcga atgcatgtat aatttctaca gaacctatta gaaaggatca |
| 541 | cccagcctct gcttttgtac aactttccct taaaaaactg ccaatcccac tgctgtttgg |
| 601 | cccaatagtg agaacttttt cctgctgcct cttggtgctt ttgcctatgg cccctattct |
| 661 | gcctgctgaa gacactcttg ccagcatgga cttaaacccc tccagctctg acaatcctct |
| 721 | ttctcttttg ttttacatga agggtctggc agccaaagca atcactcaaa gttcaaacct |
| 781 | tatcattttt tgctttgttc ctcttggcct tggttttgta catcagcttt gaaaatacca |
| 841 | tcccagggtt aatgctgggg ttaatttata actgagagtg ctctagttct gcaatacagg |
| 901 | acatgctata aaaatggaaa gatgttgctt tctgagagat cagcttacat gtggtaccga |
| 961 | gctcggatcc actagtaacg gccgccagtg tgctggaatt cggctttcca ggccatctcc |
| 1021 | aaccatggga gtgaggcacc cgccctgctc ccaccggctc ctggccgtct gcgccctcgt |
| 1081 | gtccttggca accgctgcac tcctggagtt tgggctgagc tgggtctttc tggtggccct |
| 1141 | gctgaaggga gtccagtgtg agcagcaggg agccagcaga ccagggcccc gggatgccca |
| 1201 | ggcacacccc ggccgtccca gagcagtgcc cacacagtgc gacgtccccc ccaacagccg |
| 1261 | cttcgattgc gcccctgaca aggccatcac ccaggaacag tgcgaggccc gcggctgctg |
| 1321 | ctacatccct gcaaagcagg ggctgcaggg agcccagatg gggcagccct ggtgcttctt |
| 1381 | cccacccagc taccccagct acaagctgga gaacctgagc tcctctgaaa tgggctacac |
| 1441 | ggccaccctg acccgtacca cccccacctt cttccccaag gacatcctga ccctgcggct |
| 1501 | ggacgtgatg atggagactg agaaccgcct ccacttcacg atcaaagatc cagctaacag |
| 1561 | gcgctacgag gtgcccttgg agaccccgcg tgtccacagc cgggcaccgt ccccactcta |
| 1621 | cagcgtggag ttctccgagg agcccttcgg ggtgatcgtg caccggcagc tggacggccg |
| 1681 | cgtgctgctg aacacgacgg tggcgcccct gttctttgcg gaccagttcc ttcagctgtc |
| 1741 | cacctcgctg ccctcgcagt atatcacagg cctcgccgag cacctcagtc ccctgatgct |
| 1801 | cagcaccagc tggaccagga tcaccctgtg gaaccgggac cttgcgccca cgcccggtgc |
| 1861 | gaacctctac gggtctcacc ctttctacct ggcgctggag gacggcgggt cggcacacgg |
| 1921 | ggtgttcctg ctaaacagca atgccatgga tgtggtcctg cagccgagcc ctgcccttag |
| 1981 | ctggaggtcg acaggtggga tcctggatgt ctacatcttc ctgggcccag agcccaagag |
| 2041 | cgtggtgcag cagtacctgg acgttgtggg atacccgttc atgccgccat actggggcct |
| 2101 | gggcttccac ctgtgccgct ggggctactc ctccaccgct atcacccgcc aggtggtgga |
| 2161 | gaacatgacc agggcccact tccccctgga cgtccaatgg aacgacctgg actacatgga |
| 2221 | ctcccggagg gacttcacgt tcaacaagga tggcttccgg gacttcccgg ccatggtgca |
| 2281 | ggagctgcac cagggcggcc ggcgctacat gatgatcgtg gatcctgcca tcagcagctc |
| 2341 | gggccctgcc gggagctaca ggccctacga cgagggtctg cggagggggg ttttcatcac |
| 2401 | caacgagacc ggccagccgc tgattgggaa ggtatggccc gggtccactg ccttccccga |
| 2461 | cttcaccaac cccacagccc tggcctggtg ggaggacatg gtggctgagt tccatgacca |
| 2521 | ggtgcccttc gacggcatgt ggattgacat gaacgagcct tccaacttca tcagaggctc |
| 2581 | tgaggacggc tgccccaaca atgagctgga gaacccaccc tacgtgcctg gggtggttgg |
| 2641 | ggggaccctc caggcggcca ccatctgtgc ctccagccac cagtttctct ccacacacta |
| 2701 | caacctgcac aacctctacg gcctgaccga agccatcgcc tcccacaggg cgctggtgaa |
| 2761 | ggctcggggg acacgcccat ttgtgatctc ccgctcgacc tttgctggcc acggccgata |
| 2821 | cgccggccac tggacggggg acgtgtggag ctcctgggag cagctcgcct cctccgtgcc |
| 2881 | agaaatcctg cagtttaacc tgctgggggt gcctctggtc ggggccgacg tctgcggctt |
| 2941 | cctgggcaac acctcagagg agctgtgtgt gcgctggacc cagctggggg ccttctaccc |
| 3001 | cttcatgcgg aaccacaaca gcctgctcag tctgccccag gagccgtaca gcttcagcga |
| 3061 | gccggcccag caggccatga ggaaggccct caccctgcgc tacgcactcc tcccccacct |
| 3121 | ctacacactg ttccaccagg cccacgtcgc gggggagacc gtggcccggc ccctcttcct |
| 3181 | ggagttcccc aaggactcta gcacctggac tgtggaccac cagctcctgt ggggggaggc |
| 3241 | cctgctcatc accccagtgc tccaggccgg gaaggccgaa gtgactggct acttcccctt |
| 3301 | gggcacatgg tacgacctgc agacggtgcc aatagaggcc cttggcagcc tcccaccccc |
| 3361 | acctgcagct ccccgtgagc cagccatcca cagcgagggg cagtgggtga cgctgccggc |
| 3421 | ccccctggac accatcaacg tccacctccg ggctgggtac atcatccccc tgcagggccc |
| 3481 | tggcctcaca accacagagt cccgccagca gcccatggcc ctggctgtgg ccctgaccaa |
| 3541 | gggtggagag gcccgagggg agctgttctg ggacgatgga gagagcctgg aagtgctgga |
| 3601 | gcgaggggcc tacacacagg tcatcttcct ggccaggaat aacacgatcg tgaatgagct |
| 3661 | ggtacgtgtg accagtgagg gagctggcct gcagctgcag aaggtgactg tcctgggcgt |
| 3721 | ggccacggcg ccccagcagg tcctctccaa cggtgtccct gtctccaact tcacctacag |
| 3781 | ccccgacacc aaggtcctgg acatctgtgt ctcgctgttg atgggagagc agtttctcgt |
| 3841 | cagctggtgt tagccgggcg gagtgtgtta gtctctccag agggaggctg gttccccagg |
| 3901 | gaagcagagc ctgtgtgcgg gcagcagctg tgtgcgggcc tgggggttgt taagtgcaat |
| 3961 | tatttttaat aaaaggggca tttggaaaaa aaaaaaaaag gtagcagtcg acagatgaat |
| 4021 | tctgcagatc tgtggcttct agctgcccgg gtggcatccc tgtgacccct ccccagtgcc |
| 4081 | tctcctggcc ctggaagttg ccactccagt gcccaccagc cttgtcctaa taaaattaag |
| 4141 | ttgcatcatt ttgtctgact aggtgtcctt ctataatatt atggggtgga ggggggtggt |
| 4201 | atggagcaag gggcaagttg ggaagacaac ctgagttgtt gggattccag gcatcgagta |
| 4261 | gataagtagc atggcgggtt aatcattaac tacaaggaac ccctagtgat ggagttggcc |
| 4321 | actccctctc tgcgcgctcg ctcgctcact gaggccgggc gaccaaaggt cgcccgacgc |
| 4381 | ccgggctttg cccgggcggc ctcagtgagc gagcgagcgc gcagagaggg acagatccg |

| ITR to ITR sequence of pP113, i.e., amino acids 1- 4433 of SEQ ID NO: 505. | |
|---|---|
| (SEQ ID NO: 515) | |
| (SEQ ID NO: 515) | |
| 1 | tgggccactc cctctctgcg cgctcgctcg ctcactgagg ccgcccgggc aaagcccggg |
| 61 | cgtcgggcga cctttggtcg cccggcctca gtgagcgagc gagcgcgcag agagggagtg |
| 121 | gccaactcca tcactagggg ttcctggagg ggtggagtcg tgacgtgaat tacgtcatag |
| 181 | ggttagggag gtcggccgct ctaggagtta atttttaaaa agcagtcaaa agtccaagtg |
| 241 | cccttgcgag catttactct ctctgtttgc tctggttaat aatctcagga gcacaaacat |
| 301 | tccttactag ttctaggagt taatttttaa aaagcagtca aaagtccaag tgcccttgcg |
| 361 | agcatttact ctctctgttt gctctggtta ataatctcag gagcacaaac attccttact |
| 421 | agttctagag cggccgccag tgtgctggaa ttcggctttt ttagggctgg aagctacctt |
| 481 | tgacatcatt tcctctgcga atgcatgtat aatttctaca gaacctatta gaaaggatca |
| 541 | cccagcctct gcttttgtac aactttccct taaaaaactg ccaatcccac tgctgtttgg |
| 601 | cccaatagtg agaacttttt cctgctgcct cttggtgctt ttgcctatgg cccctattct |
| 661 | gcctgctgaa gacactcttg ccagcatgga cttaaacccc tccagctctg acaatcctct |
| 721 | ttctcttttg ttttacatga agggtctggc agccaaagca atcactcaaa gttcaaacct |
| 781 | tatcattttt tgctttgttc ctcttggcct tggttttgta catcagcttt gaaaatacca |
| 841 | tcccagggtt aatgctgggg ttaatttata actgagagtg ctctagttct gcaatacagg |
| 901 | acatgctata aaaatggaaa gatgttgctt tctgagagat cagcttacat gtggtaccga |
| 961 | gctcggatcc actagtaacg gccgccagtg tgctggaatt cggctttcca ggccatctcc |
| 1021 | aaccatggga gtgaggcacc cgccctgctc ccaccggctc ctggccgtct gcgccctcgt |
| 1081 | gtccttggca accgctatgg agtttgggct gagctgggtc tttctggtgg ccctgctgaa |
| 1141 | gggagtccag tgtgagcagc agggagccag cagaccaggg ccccgggatg cccaggcaca |
| 1201 | ccccggccgt cccagagcag tgcccacaca gtgcgacgtc ccccccaaca gccgcttcga |
| 1261 | ttgcgcccct gacaaggcca tcacccagga acagtgcgag gcccgcggct gctgctacat |
| 1321 | ccctgcaaag caggggctgc agggagccca gatggggcag ccctggtgct tcttcccacc |
| 1381 | cagctacccc agctacaagc tggagaacct gagctcctct gaaatgggct acacggccac |
| 1441 | cctgacccgt accaccccca ccttcttccc caaggacatc ctgaccctgc ggctggacgt |
| 1501 | gatgatggag actgagaacc gcctccactt cacgatcaaa gatccagcta acaggcgcta |
| 1561 | cgaggtgccc ttggagaccc cgcgtgtcca cagccgggca ccgtccccac tctacagcgt |
| 1621 | ggagttctcc gaggagccct tcggggtgat cgtgcaccgg cagctggacg gccgcgtgct |
| 1681 | gctgaacacg acggtggcgc ccctgttctt tgcggaccag ttccttcagc tgtccacctc |
| 1741 | gctgccctcg cagtatatca caggcctcgc cgagcacctc agtcccctga tgctcagcac |
| 1801 | cagctggacc aggatcaccc tgtggaaccg ggaccttgcg cccacgcccg gtgcgaacct |
| 1861 | ctacgggtct caccctttct acctggcgct ggaggacggc gggtcggcac acggggtgtt |
| 1921 | cctgctaaac agcaatgcca tggatgtggt cctgcagccg agccctgccc ttagctggag |
| 1981 | gtcgacaggt gggatcctgg atgtctacat cttcctgggc ccagagccca agagcgtggt |
| 2041 | gcagcagtac ctggacgttg tgggataccc gttcatgccg ccatactggg gcctgggctt |
| 2101 | ccacctgtgc cgctggggct actcctccac cgctatcacc cgccaggtgg tggagaacat |
| 2161 | gaccagggcc cacttccccc tggacgtcca atggaacgac ctggactaca tggactcccg |
| 2221 | gagggacttc acgttcaaca aggatggctt ccgggacttc ccggccatgg tgcaggagct |
| 2281 | gcaccagggc ggccggcgct acatgatgat cgtggatcct gccatcagca gctcgggccc |
| 2341 | tgccgggagc tacaggccct acgacgaggg tctgcggagg ggggttttca tcaccaacga |
| 2401 | gaccggccag ccgctgattg ggaaggtatg gcccgggtcc actgccttcc ccgacttcac |
| 2461 | caaccccaca gccctggcct ggtgggagga catggtggct gagttccatg accaggtgcc |
| 2521 | cttcgacggc atgtggattg acatgaacga gccttccaac ttcatcagag gctctgagga |
| 2581 | cggctgcccc aacaatgagc tggagaaccc accctacgtg cctggggtgg ttggggggac |
| 2641 | cctccaggcg gccaccatct gtgcctccag ccaccagttt ctctccacac actacaacct |
| 2701 | gcacaacctc tacggcctga ccgaagccat cgcctcccac agggcgctgg tgaaggctcg |
| 2761 | ggggacacgc ccatttgtga tctcccgctc gacctttgct ggccacggcc gatacgccgg |
| 2821 | ccactggacg ggggacgtgt ggagctcctg ggagcagctc gcctcctccg tgccagaaat |
| 2881 | cctgcagttt aacctgctgg gggtgcctct ggtcggggcc gacgtctgcg gcttcctggg |
| 2941 | caacacctca gaggagctgt gtgtgcgctg gacccagctg ggggccttct accccttcat |
| 3001 | gcggaaccac aacagcctgc tcagtctgcc ccaggagccg tacagcttca gcgagccggc |
| 3061 | ccagcaggcc atgaggaagg ccctcaccct gcgctacgca ctcctccccc acctctacac |
| 3121 | actgttccac caggcccacg tcgcggggga gaccgtggcc cggcccctct tcctggagtt |
| 3181 | ccccaaggac tctagcacct ggactgtgga ccaccagctc ctgtgggggg aggccctgct |
| 3241 | catcacccca gtgctccagg ccgggaaggc cgaagtgact ggctacttcc ccttgggcac |
| 3301 | atggtacgac ctgcagacgg tgccaataga ggcccttggc agcctcccac ccccacctgc |
| 3361 | agctccccgt gagccagcca tccacagcga ggggcagtgg gtgacgctgc cggcccccct |
| 3421 | ggacaccatc aacgtccacc tccgggctgg gtacatcatc cccctgcagg gccctggcct |
| 3481 | cacaaccaca gagtcccgcc agcagcccat ggccctggct gtggccctga ccaagggtgg |
| 3541 | agaggcccga ggggagctgt tctgggacga tggagagagc ctggaagtgc tggagcgagg |
| 3601 | ggcctacaca caggtcatct tcctggccag gaataacacg atcgtgaatg agctggtacg |
| 3661 | tgtgaccagt gagggagctg gcctgcagct gcagaaggtg actgtcctgg gcgtggccac |
| 3721 | ggcgccccag caggtcctct ccaacggtgt ccctgtctcc aacttcacct acagccccga |
| 3781 | caccaaggtc ctggacatct gtgtctcgct gttgatggga gagcagtttc tcgtcagctg |
| 3841 | gtgttagccg ggcggagtgt gttagtctct ccagagggag gctggttccc cagggaagca |
| 3901 | gagcctgtgt gcgggcagca gctgtgtgcg ggcctggggg ttgttaagtg caattatttt |
| 3961 | taataaaagg ggcatttgga aaaaaaaaaa aaaggtagca gtcgacagat gaattctgca |
| 4021 | gatctgtggc ttctagctgc ccgggtggca tccctgtgac ccctccccag tgcctctcct |
| 4081 | ggccctggaa gttgccactc cagtgcccac cagccttgtc ctaataaaat taagttgcat |
| 4141 | cattttgtct gactaggtgt ccttctataa tattatgggg tggagggggg tggtatggag |
| 4201 | caaggggcaa gttgggaaga caacctgagt tgttgggatt ccaggcatcg agtagataag |
| 4261 | tagcatggcg ggttaatcat taactacaag gaacccctag tgatggagtt ggccactccc |
| 4321 | tctctgcgcg ctcgctcgct cactgaggcc gggcgaccaa aggtcgcccg acgcccgggc |
| 4381 | tttgcccggg cggcctcagt gagcgagcga gcgcgcagag agggacagat ccg |

### End of Sequences

1. A recombinant adenovirus associated (rAAV) vector comprising in its genome:
a. 5′ and 3′ AAV inverted terminal repeats (ITR) sequences, and
b. located between the 5′ and 3′ ITRs, a heterologous nucleic acid sequence encoding all or a portion of an endogenous GAA signal peptide and a heterologous signal peptide, or a heterologous signal peptide and an alpha-glucosidase (GAA) polypeptide,
wherein the GAA polypeptide comprises amino acid residues 28-952 of SEQ ID NO: 1, 57-952 of SEQ ID NO: 1, or comprises a N-terminal GAA polypeptide fragment comprising amino acids 28, 28-29, 28-30, 28-31, 28-32, or 28-33 of SEQ ID NO: 1 and a deletion of any number of amino acids from the next about 5 amino acids to about 40 amino acids after the N terminal GAA polypeptide fragment of SEQ ID NO: 1, or a functional fragment thereof, and
wherein the nucleic acid sequence encoding the GAA polypeptide can be codon optimized, and wherein the heterologous nucleic acid is operatively linked to a liver-specific promoter.

2. The recombinant AAV vector of claim 1, where the codon optimized nucleic acid sequence encoding the GAA polypeptide is selected from the group consisting of SEQ ID NO: 1-18, or functional fragment thereofs.

3. The recombinant AAV vector of claim 1, wherein the nucleic acid encoding SEQ ID NO: 3 is wildtype.

4. The recombinant AAV vector of claim 1, further comprising at least one of a UTR or a reverse RNA polII terminator sequence.

5. The recombinant AAV vector of claim 1, which comprises the nucleic acid sequence of SEQ ID NO: 23, or a functional variant thereof.

6. The recombinant AAV vector of claim 1, wherein the heterologous nucleic acid sequence encodes a GAA protein comprising a signal peptide fused to the GAA polypeptide, wherein the signal peptide is an endogenous GAA signal peptide, or a heterologous signal peptide, or a combination thereof.

7. The recombinant AAV vector of claim 1, wherein the AAV genome comprises, in the 5′ to 3′ direction:
a. a 5′ ITR,
b. a liver-specific promoter sequence,
c. an 5′ UTR sequence,
d. a nucleic acid encoding a portion or all of the endogenous GAA signal peptide and a heterologous signal peptide or a heterologous signal peptide,
e. a nucleic acid encoding an alpha-glucosidase (GAA) polypeptide, wherein the GAA polypeptide is functionally active,
f. a poly A sequence, and
g. a reverse RNA pol II terminator sequence.

8. The recombinant AAV vector of claim 1, wherein the UTR is 5′ or 3′.

9. The recombinant AAV vector of claim 1, wherein the nucleic acid encoding the heterologous signal peptide is selected from any of: an endogenous GAA signal peptide, a fibronectin signal peptide (FN1), a IL-2 wt signal peptide, modified IL-2 signal peptide, IL2(1-3) signal peptide, IgG signal peptide, a AAT signal peptide, a A2M signal peptide, or a PZP signal peptide, or an active fragment thereof having signal peptide activity.

10. The recombinant AAV vector of any of claim 1 or 9, wherein the nucleic acid sequence encodes a GAA polypeptide having the amino acid sequence of SEQ ID NO: 1, or a polypeptide having at least 80% sequence identity to SEQ ID NO: 1 where amino acid residue 199 is a R (199R), amino acid residue 223 is a H (223H) and amino acid residue 780 is a 1(780I).

11.-16. (canceled)

17. The recombinant AAV vector of claim 7, further comprising at least one polyA sequence located 3′ of the nucleic acid encoding the GAA gene and 5′ of the 3′ ITR sequence.

18. The recombinant AAV vector of claim 17, wherein the heterologous nucleic acid sequence further comprises a 3′ UTR sequence, wherein the 3′ UTR sequence is located 3′ of the nucleic acid encoding the GAA polypeptide and 5′ of the 3′ ITR sequence, or is located between the nucleic acid encoding the GAA polypeptide and the poly A sequence, or RNA pol II terminator sequence.

19.-23. (canceled)

24. The recombinant AAV vector of claim 17, wherein the nucleic acid encoding the GAA polypeptide encodes a GAA polypeptide beginning at any of amino acid residues 35, 40, 50, 57, 60, 68, 69, 70, 72, 74, 779, 790, 791, 792, 793, or 796 of SEQ ID NO: 1 or a sequence 80% identical to SEQ ID NO: 1 where amino acid residue 199 is a R (199R), amino acid residue 223 is a H (223H) and amino acid residue 780 is a 1(780I).

25. (canceled)

26. The recombinant AAV vector of claim 1, wherein the liver specific promoter is selected from any of: SEQ ID NOS: 86, 88, 91-96, 146-150 or 439-441, or a liver specific promoter having at least 80% sequence identity to SEQ ID NOs: 86, 88, 91-96, 146-150 or 439-441.

27.-32. (canceled)

33. The recombinant AAV vector of claim 1, wherein the recombinant AAV vector is selected from the group consisting of: a AAVXL32 vector, a AAVXL32.1 vector, a AAV8 vector, or a haploid AAV8 vector comprising at least one AAV8 capsid protein.

34.-39. (canceled)

40. A pharmaceutical composition comprising the recombinant AAV vector of claim 1 in a pharmaceutically acceptable carrier.

41. A method to treat a subject with Pompe Disease, or a a glycogen storage disease type II (GSD II, Acid Maltase Deficiency) or having a deficiency in alpha-glucosidase (GAA) polypeptide, comprising administering any of the recombinant AAV vector, or the rAAV genome or the nucleic acid sequence of claim 1.

42.-61. (canceled)
